(19) United States
(12) Patent Application Publication
(10) Pub. No.: US 2021/0261512 A1
(43) Pub. Date: Aug. 26, 2021

(54) NOVEL COMPOUNDS

(71) Applicant: INFLAZOME LIMITED, DUBLIN 2 (IE)

(72) Inventors: David MILLER, Cambridge (GB); Angus MACLEOD, Cambridge (GB); Stephen THOM, Nottingham (GB); Christopher G. MCPHERSON, Nottingham (GB); Thomas ALANINE, Nottingham (GB); Jokin CARRILLO ARREGUI, Nottingham (GB); Claire-Lise CIANA, Nottingham (GB); Jonathan SHANNON, Nottingham (GB); Jimmy VAN WILTENBURG, Groningen (NL); Jacobus Antonius Joseph DEN HARTOG, Groningen (NL)

(73) Assignee: INFLAZOME LIMITED, DUBLIN 2 (IE)

(21) Appl. No.: 17/052,512

(22) PCT Filed: May 3, 2019
(86) PCT No.: PCT/EP2019/061439

§ 371 Date: Nov. 2, 2020

(30) Foreign Application Priority Data
May 4, 2018 (GB) 1807362.7
Feb. 20, 2019 (GB) 1902329.0
Apr. 12, 2019 (GB) 1905245.5

(51) Int. Cl. C07D 249/14; C07D 403/12; C07D 405/14; C07D 249/12; C07D 401/14; C07D 401/12; C07D 413/12; C07D 405/12; C07D 487/04; C07D 487/10; C07D 491/107; C07D 401/10; A61P 37/06

(52) U.S. Cl. CPC C07D 249/14; C07D 403/12; C07D 405/14; C07D 249/12; C07D 401/14; C07D 401/12; A61P 37/06; C07D 405/12; C07D 487/04; C07D 487/10; C07D 491/107; C07D 401/10; C07D 413/12

(57) ABSTRACT

The present invention relates to substituted 5-membered nitrogen containing heteroaryl compounds, such as sulfonyl triazoles, where the heteroaryl ring is further substituted, optionally via a linking group such as —NH—, with a cyclic group which in turn is substituted at the α-position. The present invention further relates to associated salts, solvates, prodrugs and pharmaceutical compositions, and to the use of such compounds in the treatment and prevention of medical disorders and diseases, most especially by NLRP₃ inhibition.

### FIELD OF THE INVENTION

The present invention relates to substituted 5-membered nitrogen containing heteroaryl compounds, such as sulfonyl triazoles, where the heteroaryl ring is further substituted, optionally via a linking group such as —NH—, with a cyclic group which in turn is substituted at the α-position. The present invention further relates to associated salts, solvates, prodrugs and pharmaceutical compositions, and to the use of such compounds in the treatment and prevention of medical disorders and diseases, most especially by NLRP3 inhibition.

### BACKGROUND

The NOD-like receptor (NLR) family, pyrin domain-containing protein 3 (NLRP3) inflammasome is a component of the inflammatory process, and its aberrant activity is pathogenic in inherited disorders such as cryopyrin-associated periodic syndromes (CAPS) and complex diseases such as multiple sclerosis, type 2 diabetes, Alzheimer's disease and atherosclerosis.

NLRP3 is an intracellular signalling molecule that senses many pathogen-derived, environmental and host-derived factors. Upon activation, NLRP3 binds to apoptosis-associated speck-like protein containing a caspase activation and recruitment domain (ASC). ASC then polymerises to form a large aggregate known as an ASC speck. Polymerised ASC in turn interacts with the cysteine protease caspase-1 to form a complex termed the inflammasome. This results in the activation of caspase-1, which cleaves the precursor forms of the proinflammatory cytokines IL-1β and IL-18 (termed pro-IL-1 and pro-IL-18 respectively) to thereby activate these cytokines. Caspase-1 also mediates a type of inflammatory cell death known as pyroptosis. The ASC speck can also recruit and activate caspase-8, which can process pro-IL-1 and pro-IL-18 and trigger apoptotic cell death.

Caspase-1 cleaves pro-IL-1 and pro-IL-18 to their active forms, which are secreted from the cell. Active caspase-1 also cleaves gasdermin-D to trigger pyroptosis. Through its control of the pyroptotic cell death pathway, caspase-1 also mediates the release of alarmin molecules such as IL-33 and high mobility group box 1 protein (HMGBi). Caspase-1 also cleaves intracellular IL-1R2 resulting in its degradation and allowing the release of IL-1α. In human cells caspase-1 may also control the processing and secretion of IL-37. A number of other caspase-1 substrates such as components of the cytoskeleton and glycolysis pathway may contribute to caspase-1-dependent inflammation.

NLRP3-dependent ASC specks are released into the extracellular environment where they can activate caspase-1, induce processing of caspase-1 substrates and propagate inflammation.

Active cytokines derived from NLRP3 inflammasome activation are important drivers of inflammation and interact with other cytokine pathways to shape the immune response to infection and injury. For example, IL-1 signalling induces the secretion of the pro-inflammatory cytokines IL-6 and TNF. IL-1β and IL-18 synergise with IL-23 to induce IL-17 production by memory CD4 Th17 cells and by γδ T cells in the absence of T cell receptor engagement. IL-18 and IL-12 also synergise to induce IFN-γ production from memory T cells and NK cells driving a Thi response.

The inherited CAPS diseases Muckle-Wells syndrome (MWS), familial cold autoinflammatory syndrome (FCAS) and neonatal-onset multisystem inflammatory disease (NOMID) are caused by gain-of-function mutations in NLRP3, thus defining NLRP3 as a critical component of the inflammatory process. NLRP3 has also been implicated in the pathogenesis of a number of complex diseases, notably including metabolic disorders such as type 2 diabetes, atherosclerosis, obesity and gout.

A role for NLRP3 in diseases of the central nervous system is emerging, and lung diseases have also been shown to be influenced by NLRP3. Furthermore, NLRP3 has a role in the development of liver disease, kidney disease and aging. Many of these associations were defined using Nlr3−/− mice, but there have also been insights into the specific activation of NLRP3 in these diseases. In type 2 diabetes mellitus (T2D), the deposition of islet amyloid polypeptide in the pancreas activates NLRP3 and IL-1p signaling, resulting in cell death and inflammation.

Several small molecules have been shown to inhibit the NLRP3 inflammasome. Glyburide inhibits IL-1β production at micromolar concentrations in response to the activation of NLRP3 but not NLRC4 or NLRP1. Other previously characterised weak NLRP3 inhibitors include parthenolide, 3,4-methylenedioxy-β-nitrostyrene and dimethyl sulfoxide (DMSO), although these agents have limited potency and are nonspecific.

Current treatments for NLRP3-related diseases include biologic agents that target IL-1. These are the recombinant IL-1 receptor antagonist anakinra, the neutralizing IL-1β antibody canakinumab and the soluble decoy IL-1 receptor rilonacept. These approaches have proven successful in the treatment of CAPS, and these biologic agents have been used in clinical trials for other IL-1β-associated diseases.

Certain sulfonylurea-containing compounds are also disclosed as inhibitors of NLRP3 (see for example, Baldwin et al., J. Med. Chem., 59 (5), 1691-1710, 2016; and WO 2016/131098 A1, WO 2017/129897 A1, WO 2017/140778 A1, WO 2017/184604 A1, WO 2017/184623 A1, WO 2017/184624 A1, WO 2018/136890 A1, WO 2018/015445 A1, WO 2018/215818 A1, WO 2019/008029 A1, WO 2019/008025 A1, WO 2019/034697 A1, WO 2019/034696 A1, WO 2019/034686 A1, WO 2019/034688 A1, WO 2019/034690 A1, WO 2019/034692 A1 and WO 2019/034693 A1). In addition, WO 2019/068772 A1 discloses a number of sulfoximine ureas as inhibitors of NLRP3.

Certain heterocyclic sulfonyl compounds, including a number of sulfonyl thiadiazole and sulfonyl oxadiazole compounds, have been suggested as inhibitors of IL-8 (see for example GB 2 379 218 A and GB 2 380 190 A). However, IL-8 secretion is controlled by routes other than NLRP-3 activation.

There is a need to provide compounds with improved pharmacological and/or physiological and/or physicochemical properties and/or those that provide a useful alternative to known compounds.

### SUMMARY OF THE INVENTION

A first aspect of the invention provides a compound of formula (I):

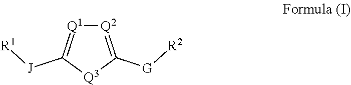

wherein:
Q¹ and Q² are each independently selected from N or CR^{q}, provided that at least one of Q¹ and Q² is N;
Q³ is O, S or NR^{qq};
each R^{q} is independently selected from hydrogen or a halo, —OH, —NO₂, —NH₂, —N₃, —SH, —SO₂H, —SO₂NH₂, or a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton;
each R^{qq} is independently selected from hydrogen or a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton;
J is a bond, —O—, —S—, —SO—, —SO₂—, —SO(═NR^{jj})—, —CO—, —C(═S)—, —C(R^{j})₂—, —C(═C(R^{jjj})₂)—, —C(═NR^{jj})—, —NR^{jj}—, —O—C(R^{jjj})₂—, —O—C(═C(R^{jjj})₂)—, —O—C(═NR^{jj})—, —S—C(R^{j})₂—, —S—C(═C(R^{jjj})₂)—, —S—C(═NR^{jj})—, —SO—C(R^{j})₂—, —SO—C(═C(R^{jjj})₂)—, —SO—C(═NR^{jj})—, —SO—NR^{jj}—, —SO₂—C(R^{j})₂—, —SO₂—C(═C(R^{jjj})₂)—, —SO₂—C(═NR^{jj})—, —SO₂—NR^{jj}—, —SO(═NR^{jj})—C(R^{j})₂—, —SO(═NR^{jj})—C(═C(R^{jjj})₂)—, —SO(═NR^{jj})—NR—, —CO—C(R^{j})₂—, —CO—C(═C(R^{jjj})₂)—, —CO—C(═NR^{jj})—, —CO—NR^{jj}—, —C(═S)—C(R^{j})₂—, —C(═S)—C(═C(R^{jjj})₂)—, —C(R^{j})₂—O—, —C(R^{j})₂—S—, —C(R^{j})₂—SO—, —C(R^{j})₂—SO₂—, —C(R^{j})₂—SO(═NR^{jj})—, —C(R^{j})₂—CO—, —C(R^{j})₂—C(═S)—, —C(R^{j})₂—C(R^{j})₂—, —C(R^{j})₂—C(═C(R^{jjj})₂)—, —C(R^{j})₂—C(═NR^{jj})—, —C(R^{j})₂—NR^{jj}—, —C(═C(R^{jjj})₂)—O—, —C(═C(R^{jjj})₂)—S—, —C(═C(R^{jjj})₂)—SO—, —C(═C(R^{jjj})₂)—SO₂—, —C(═C(R^{jjj})₂)—SO(═NR^{jj})—, —C(═C(R^{jjj})₂)—CO—, —C(═C(R^{jjj})₂)—C(═S)—, —C(═C(R^{jj})₂)—C(R^{j})₂—, —C(═C(R^{jj})₂)—C(═C(R^{jjj})₂)—, —C(═C(R^{jj})₂)—C(═NR^{jj})—, —C(═C(R^{jjj})₂)—NR^{jj}—, —C(═NR^{jj})—O—, —C(═NR^{jj})—S—, —C(═NR^{jj})—SO—, —C(═NR^{jj})—SO₂—, —C(═NR^{jj})—CO—, —C(═NR^{jj})—C(R^{j})₂—, —C(═NR^{jj})—C(═C(R^{jjj})₂)—, —C(═NR^{jj})—C(═NR^{jj})—, —C(═NR^{jj})—NR^{jj}—, —NR^{jj}—SO—, —NR^{jj}—SO₂—, —NR^{jj}—SO(═NR^{jj})—, —NR^{jj}—CO—, —NR—C(R^{j})₂—, —NR^{jj}—C(═C(R^{jjj})₂)—, —NR^{jj}—C(═NR^{jj})—, —CR^{jjj}═CR^{jjj}—, —CR^{jjj}═N—, —N═CR^{jjj}— or —C≡C—;
each R^{j} is independently selected from hydrogen or a halo, —OH, —NO₂, —NH₂, —N₃, —SH, —SO₂H, —S₂NH₂, or a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton;
each R^{jj} is independently selected from hydrogen or a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton;
each R^{jjj} is independently selected from hydrogen or a halo or a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton;
or wherein optionally any two or three R^{j}, any two R^{jj}, any two or three R^{jjj}, or any two or three of R^{j}, R^{jj} and R^{jjj}, together with the atom or atoms to which they are attached, may form a saturated or unsaturated cyclic group, wherein the cyclic group may optionally be substituted;
R¹ is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton;
G is a bond, —O—, —S—, —SO—, —SO₂—, —SO(═NR^{gg})—, —CO—, —C(═S)—, —C(R^{g})₂—, —C(═C(R^{ggg})₂)—, —C(═NR^{gg})—, —NR^{gg}—, —O—C(R^{g})₂—, —O—C(═C(R^{ggg})₂)—, —O—C(═NR^{gg})—, —S—C(R^{g})₂—, —S—C(═C(R^{ggg})₂)—, —S—C(═NR^{gg})—, —SO—C(R^{g})₂—, —SO—C(═C(R^{ggg})₂)—, —SO—C(═NR^{gg})—, —SO—NR^{gg}—, —SO₂—C(R^{g})₂—, —SO₂—C(═C(R^{gg})₂)—, —SO₂—C(═NR^{gg})—, —SO₂—NR^{gg}—, —SO(═NR^{gg})—C(R^{g})₂—, —SO(═NR^{gg})—C(═C(R^{ggg})₂)—, —SO(═NR^{gg})—NR^{gg}—, —CO—C(R^{g})₂—, —CO—C(═C(R^{ggg})₂)—, —CO—C(═NR^{gg})—, —CO—NR^{gg}—, —C(═S)—C(R^{g})₂—, —C(═S)—C(═C(R^{ggg})₂)—, —C(R^{g})₂—O—, —C(R^{g})₂—S—, —C(R^{g})₂—SO—, —C(R^{g})₂—SO₂—, —C(R^{g})₂—SO(═NR^{gg})—, —C(R^{g})₂—CO—, —C(R^{g})₂—C(═S)—, —C(R^{g})₂—C(R^{g})₂—, —C(R^{g})₂—C(═C(R^{ggg})₂)—, —C(R^{g})₂—C(═NR^{gg})—, —C(R^{g})₂—NR^{gg}—, —C(═C(R^{ggg})₂)—O—, —C(═C(R^{ggg})₂)—S—, —C(═C(R^{ggg})₂)—SO—, —C(═C(R^{ggg})₂)—SO₂—, —C(═C(R^{ggg})₂)—SO(═NR^{gg})—, —C(═C(R^{ggg})₂)—CO—, —C(═C(R^{ggg})₂)—C(═S)—, —C(═C(R^{ggg})₂)—C(R^{g})₂—, —C(═C(R^{ggg})₂)—C(═C(R^{ggg})₂)—, —C(═C(R^{ggg})₂)—C(═NR^{gg})—, —C(═C(R^{ggg})₂)—NR^{gg}—, —C(═NR^{gg})—O—, —C(═NR^{gg})—S—, —C(═NR^{gg})—SO—, —C(═NR^{gg})—SO₂—, —C(═NR^{gg})—CO—, —C(═NR^{gg})—C(R^{g})₂—, —C(═NR^{gg})—C(═C(R^{ggg})₂)—, —C(═NR^{gg})—C(═NR^{gg})—, —C(═NR^{gg})—NR^{gg}—, —NR^{gg}—SO—, —NR^{gg}—SO₂—, —NR^{gg}—SO(═NR^{gg})—, —NR^{gg}—CO—, —NR^{gg}—C(R^{g})₂—, —NR^{gg}—C(═C(R^{ggg})₂)—, —NR^{gg}—C(═NR^{gg})—, —CR^{ggg}═CR^{ggg}—, —CR^{ggg}═N—, —N═CR^{ggg}— or —C≡C—;
each R^{g} is independently selected from hydrogen or a halo, —OH, —NO₂, —NH₂, —N₃. —SH. —SO₂H. —SO₂NH₂. or a saturated or unsaturated hydrocarbyl group. wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton;
each R^{gg} is independently selected from hydrogen or a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton;
each R^{ggg} is independently selected from hydrogen or a halo or a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton;
or wherein optionally any two or three R^{g}, any two R^{gg}, any two or three R^{ggg}, or any two or three of R^{g}, R^{gg} and R^{ggg}, together with the atom or atoms to which they are attached, may form a saturated or unsaturated cyclic group, wherein the cyclic group may optionally be substituted; and
R² is a cyclic group substituted at the α-position, wherein R² may optionally be further substituted.

In the context of the present specification, a “hydrocarbyl” substituent group or a hydrocarbyl moiety in a substituent group only includes carbon and hydrogen atoms but, unless stated otherwise, does not include any heteroatoms, such as N, O or S, in its carbon skeleton. A hydrocarbyl group/moiety may be saturated or unsaturated (including aromatic), and may be straight-chained or branched, or be or include cyclic groups wherein, unless stated otherwise, the cyclic group does not include any heteroatoms, such as N, O or S, in its carbon skeleton. Examples of hydrocarbyl groups include alkyl, alkenyl, alkynyl, cycloalkyl, cycloalkenyl and aryl groups/moieties and combinations of all of these groups/moieties. Typically a hydrocarbyl group is a C₁-C₂ hydrocarbyl group. More typically a hydrocarbyl group is a C₁-C₁₅ hydrocarbyl group. More typically a hydrocarbyl group is a C₁-C₁₀ hydrocarbyl group. A “hydrocarbylene” group is similarly defined as a divalent hydrocarbyl group.

An “alkyl” substituent group or an alkyl moiety in a substituent group may be linear (i.e. straight-chained) or branched. Examples of alkyl groups/moieties include methyl, ethyl, n-propyl, i-propyl, n-butyl, i-butyl, t-butyl and n-pentyl groups/moieties. Unless stated otherwise, the term “alkyl” does not include “cycloalkyl”. Typically an alkyl group is a C₁-C₁₂ alkyl group. More typically an alkyl group is a C₁-C₆ alkyl group. An “alkylene” group is similarly defined as a divalent alkyl group.

An “alkenyl” substituent group or an alkenyl moiety in a substituent group refers to an unsaturated alkyl group or moiety having one or more carbon-carbon double bonds. Examples of alkenyl groups/moieties include ethenyl, propenyl, 1-butenyl, 2-butenyl, 1-pentenyl, 1-hexenyl, 1,3-butadienyl, 1,3-pentadienyl, 1,4-pentadienyl and 1,4-hexadienyl groups/moieties. Unless stated otherwise, the term “alkenyl” does not include “cycloalkenyl”. Typically an alkenyl group is a C₂-C₁₂ alkenyl group. More typically an alkenyl group is a C₂-C₆ alkenyl group. An “alkenylene” group is similarly defined as a divalent alkenyl group.

An “alkynyl” substituent group or an alkynyl moiety in a substituent group refers to an unsaturated alkyl group or moiety having one or more carbon-carbon triple bonds. Examples of alkynyl groups/moieties include ethynyl, propargyl, but-1-ynyl and but-2-ynyl groups/moieties. Typically an alkynyl group is a C₂-C₁₂ alkynyl group. More typically an alkynyl group is a C₂-C₆ alkynyl group. An “alkynylene” group is similarly defined as a divalent alkynyl group.

A “cyclic” substituent group or a cyclic moiety in a substituent group refers to any hydrocarbyl ring, wherein the hydrocarbyl ring may be saturated or unsaturated (including aromatic) and may include one or more heteroatoms, e.g. N, O or S, in its carbon skeleton. Examples of cyclic groups include cycloalkyl, cycloalkenyl, heterocyclic, aryl and heteroaryl groups as discussed below. A cyclic group may be monocyclic, bicyclic (e.g. bridged, fused or spiro), or polycyclic. Typically, a cyclic group is a 3- to 12-membered cyclic group, which means it contains from 3 to 12 ring atoms. More typically, a cyclic group is a 3- to 7-membered monocyclic group, which means it contains from 3 to 7 ring atoms.

As used herein, where it is stated that a cyclic group is monocyclic, it is to be understood that the cyclic group is not substituted with a divalent bridging substituent (e.g. —O—, —S—, —NH—, —N(R^{β})—, —N(O)(R^{β})—, —N⁺(R^{β})₂— or —R^{α}—) so as to form a bridged, fused or spiro substituent. However, unless stated otherwise, a substituted monocyclic group may be substituted with one or more monovalent cyclic groups. Similarly, where it is stated that a group is bicyclic, it is to be understood that the cyclic group including any bridged, fused or spiro divalent bridging substituents attached to the cyclic group, but excluding any monovalent cyclic substituents, is bicyclic.

A “heterocyclic” substituent group or a heterocyclic moiety in a substituent group refers to a cyclic group or moiety including one or more carbon atoms and one or more (such as one, two, three or four) heteroatoms, e.g. N, O or S, in the ring structure. Examples of heterocyclic groups include heteroaryl groups as discussed below and non-aromatic heterocyclic groups such as azetinyl, azetidinyl, oxetanyl, thietanyl, pyrrolidinyl, tetrahydrofuranyl, tetrahydrothiophenyl, pyrazolidinyl, imidazolidinyl, dioxolanyl, oxathiolanyl, piperidinyl, tetrahydropyranyl, thianyl, piperazinyl, dioxanyl, morpholinyl and thiomorpholinyl groups.

A “cycloalkyl” substituent group or a cycloalkyl moiety in a substituent group refers to a saturated hydrocarbyl ring containing, for example, from 3 to 7 carbon atoms, examples of which include cyclopropyl, cyclobutyl, cyclopentyl and cyclohexyl. Unless stated otherwise, a cycloalkyl substituent group or moiety may include monocyclic, bicyclic or polycyclic hydrocarbyl rings.

A “cycloalkenyl” substituent group or a cycloalkenyl moiety in a substituent group refers to a non-aromatic unsaturated hydrocarbyl ring having one or more carbon-carbon double bonds and containing, for example, from 3 to 7 carbon atoms, examples of which include cyclopent-1-en-1-yl, cyclohex-1-en-1-yl and cyclohex-1,3-dien-1-yl. Unless stated otherwise, a cycloalkenyl substituent group or moiety may include monocyclic, bicyclic or polycyclic hydrocarbyl rings.

An “aryl” substituent group or an aryl moiety in a substituent group refers to an aromatic hydrocarbyl ring. The term “aryl” includes monocyclic aromatic hydrocarbons and polycyclic fused ring aromatic hydrocarbons wherein all of the fused ring systems (excluding any ring systems which are part of or formed by optional substituents) are aromatic. Examples of aryl groups/moieties include phenyl, naphthyl, anthracenyl and phenanthrenyl. Unless stated otherwise, the term “aryl” does not include “heteroaryl”.

A “heteroaryl” substituent group or a heteroaryl moiety in a substituent group refers to an aromatic heterocyclic group or moiety. The term “heteroaryl” includes monocyclic aromatic heterocycles and polycyclic fused ring aromatic heterocycles wherein all of the fused ring systems (excluding any ring systems which are part of or formed by optional substituents) are aromatic. Examples of heteroaryl groups/moieties include the following:

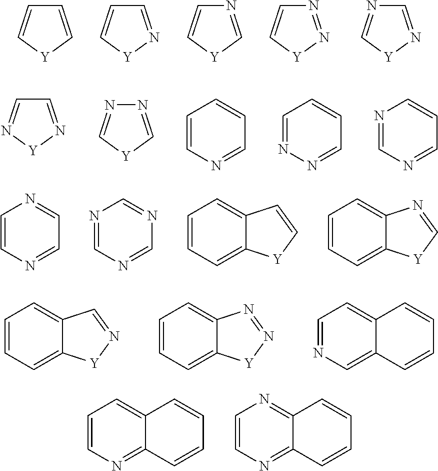

wherein Y═O, S or NH.

Unless stated otherwise, where a cyclic group or moiety is stated to be non-aromatic, such as a cycloalkyl, cycloalkenyl or non-aromatic heterocyclic group, it is to be understood that the group or moiety, excluding any ring systems which are part of or formed by optional substituents, is non-aromatic. Similarly, where a cyclic group or moiety is stated to be aromatic, such as an aryl or a heteroaryl group, it is to be understood that the group or moiety, excluding any ring systems which are part of or formed by optional substituents, is aromatic. A cyclic group or moiety is considered non-aromatic, when it does not have any tautomers that are aromatic. When a cyclic group or moiety has a tautomer that is aromatic, it is considered aromatic, even if it has tautomers that are not aromatic.

By way of example, the following are considered aromatic heterocyclic groups, because they have an aromatic tautomer:

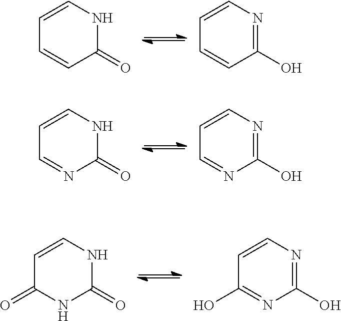

For the avoidance of doubt, the term “non-aromatic heterocyclic group” does not exclude heterocyclic groups or moieties which may possess aromatic character only by virtue of mesomeric charge separation.

For example, the following is considered a non-aromatic heterocyclic group, because it does not have an aromatic tautomer:

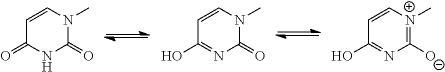

because the last shown structure is not taken into consideration because of mesomeric charge separation.

For the purposes of the present specification, where a combination of moieties is referred to as one group, for example, arylalkyl, arylalkenyl, arylalkynyl, alkylaryl, alkenylaryl or alkynylaryl, the last mentioned moiety contains the atom by which the group is attached to the rest of the molecule. An example of an arylalkyl group is benzyl.

For the purposes of the present specification, in an optionally substituted group or moiety:

(i) each hydrogen atom may optionally be replaced by a group independently selected from halo; —CN; —NO₂; —N₃; —R^{β}; —OH; —OR^{β}; —R^{α}-halo; —R^{α}—CN; —R^{α}—NO₂; —R^{α}—N₃; —R^{α}—R^{β}; —R^{α}—OH; —R^{α}—OR^{β}; —SH; —SR^{β}; —SOR^{β}; —SO₂H; —SO₂R^{β}; —SO₂NH₂; —SO₂NHR^{β}; —SO₂N(R^{β})₂; —R^{α}—SH; —R^{α}—SR^{β}; —R^{α}—SOR^{β}; —R^{α}—SO₂H; —R^{α}—SO₂R^{β}; —R^{α}—SO₂NH₂; —R^{α}—SO₂NHR^{β}; —R^{α}—SO₂N(R^{β})₂; —Si(R^{β})₃; —O—Si(R^{β})₃; —R^{α}—Si(R^{β})₃; —R^{α}—O—Si(R^{β})₃; —NH₂; —NHR^{β}; —N(R^{β})₂; —N(O)(R^{β})₂; —N⁺(R^{β})₃; —R^{α}—NH₂; —R^{α}—NHR^{β}; —R^{α}—N(R^{β})₂; —R^{α}—N(O)(R^{β})₂; —R^{α}—N⁺(R^{β})₃; —CHO; —COR^{β}; —COOH; —COOR^{β}; —OCOR^{β}; —R^{α}—CHO; —R^{α}—COR^{β}; —R^{α}—COOH; —R^{α}—COOR^{β}; —R^{α}—OCOR^{β}; —C(═NH)R^{β}; —C(═NH)NH₂; —C(═NH)NHR^{β}; —C(═NH)N(R^{β})₂; —C(═NR^{β})R^{β}; —C(═NR^{β})NHR^{β}; —C(═NR^{β})N(R^{β})₂; —C(═NOH)R^{β}; —C(═NOR^{β})R^{β}; —C(N₂)R^{β}; —R^{α}—C(═NH)R^{β}; —R^{α}—C(═NH)NH₂; —R^{α}—C(═NH^{β})NHR^{β}; —R^{α}—C(═NH)N(R^{β})₂; —R^{α}—C(═NR^{β})R^{β}; —R^{α}—C(═NR^{β})NHR^{β}; —R^{α}—C(═NR^{β})N(R^{β})₂; —R^{α}—C(═NOH)R^{β}; —R^{α}—C(═NOR^{β})R^{β}; —R^{α}—C(N₂)R^{β}; —NH—CHO; —NR^{β}—CHO; —NH—COR^{β}; —NR^{β}—COR^{β}; —CONH₂; —CONHR^{β}; —CON(R^{β})₂; —R^{α}—NH—CHO; —R^{α}—NR^{β}—CHO; —R^{α}—NH—COR^{β}; —R^{α}—NR^{β}—COR^{β}; —R^{α}—CONH₂; —R^{α}—CONHR^{β}; —R^{α}—CON(R^{β})₂; —O—R^{α}—OH; —O—R^{α}—OR^{β}; —O—R^{α}—NH₂; —O—R^{α}—NHR^{β}; —O—R^{α}—N(R^{β})₂; —O—R^{α}—N(O)(R^{β})₂; —O—R^{α}—N⁺(R^{β})₃; —NH—R^{α}—OH: —NH—R^{α}—OR^{β}: —NH—R^{α}—NH₂: —NH—R^{α}—NHR^{β}; —NH—R^{α}—N(R^{β})₂; —NH—R^{α}—N(O)(R^{β})₂; —NH—R^{α}—N⁺(R^{β})₃; —NR^{β}—R^{α}—OH; —NR^{β}—R^{α}—OR^{β}; —NR^{β}—R^{α}—NH₂; —NR^{β}—R^{α}—NHR^{β}; —NR^{β}—R^{α}—N(R^{β})₂; —NR^{β}—R^{α}—N(O)(R^{β})₂; —NR^{β}—R^{α}—N⁺(R^{β})₃; —N(O)R^{β}—R^{α}—OH; —N(O)R^{β}—R^{α}—OR^{β}; —N(O)R^{β}—R^{α}—NH₂; —N(O)R^{β}—R^{α}—NHR^{β}; —N(O)R^{β}—R^{α}—N(R^{β})₂; —N(O)R^{β}—R^{α}—N(O)(R^{β})₂; —N(O)R^{β}—R^{α}—N⁺(R^{β})₃; —N⁺(R^{β})₂—R^{α}—OH; —N⁺(R^{β})₂—R^{α}—OR^{β}; —N⁺(R^{β})₂—R^{α}—NH₂; —N⁺(R^{β})₂—R^{α}—NHR^{β}; —N⁺(R^{β})₂—R^{α}—N(R^{β})₂; or —N⁺(R^{β})₂—R^{α}—N(O)(R^{β})₂; and/or

(ii) any two hydrogen atoms attached to the same carbon or nitrogen atom may optionally be replaced by a π-bonded substituent independently selected from oxo (═O), ═S, ═NH or ═NR^{β}; and/or

(iii) any sulfur atom may optionally be substituted with one or two π-bonded substituents independently selected from oxo (═O), ═NH or ═NR^{β}; and/or

(iv) any two hydrogen atoms attached to the same or different atoms, within the same optionally substituted group or moiety, may optionally be replaced by a bridging substituent independently selected from —O—, —S—, —NH—, —N═N—, —N(R^{β})—, —N(O)(R^{β})—, —N⁺(R^{β})₂— or —R^{α}—;
wherein each —R^{α}— is independently selected from an alkylene, alkenylene or alkynylene group, wherein the alkylene, alkenylene or alkynylene group contains from 1 to 6 atoms in its backbone, wherein one or more carbon atoms in the backbone of the alkylene, alkenylene or alkynylene group may optionally be replaced by one or more heteroatoms N, O or S, wherein one or more —CH₂— groups in the backbone of the alkylene, alkenylene or alkynylene group may optionally be replaced by one or more —N(O)(R^{β})— or —N⁺(R^{β})₂— groups, and wherein the alkylene, alkenylene or alkynylene group may optionally be substituted with one or more halo and/or —R^{β} groups; and
wherein each —R^{β} is independently selected from a C₁-C₆ alkyl, C₂-C₆ alkenyl, C₂-C₆ alkynyl or C₂-C₆ cyclic group, or wherein any two or three —R^{β} attached to the same nitrogen atom may, together with the nitrogen atom to which they are attached, form a C₂-C₇ cyclic group, and wherein any —R^{β} may optionally be substituted with one or more C₁-C₄ alkyl, C₁-C₄ haloalkyl, C₃-C₇ cycloalkyl, C₃-C₇ halocycloalkyl, —O(C₁-C₄ alkyl), —O(C₁-C₄ haloalkyl), —O(C₃-C₇ cycloalkyl), —O(C₃-C₇ halocycloalkyl), —CO(C₁-C₄ alkyl), —CO(C₁-C₄ haloalkyl), —CO(C₃-C₇ cycloalkyl), —CO(C₃-C₇ halocycloalkyl), —COO(C₁-C₄ alkyl), —COO(C₁-C₄ haloalkyl), —COO(C₃-C₇ cycloalkyl), —COO(C₃-C₇ halocycloalkyl), halo, —OH, —NH₂, —CN, —C≡CH, oxo (═O), or 4- to 6-membered heterocyclic group.

Typically, the compounds of the present invention comprise at most one quaternary ammonium group such as —N⁺(R^{β})₃ or —N⁺(R^{β})₂-.

Where reference is made to a —R^{α}—C(N₂)R^{β} group, what is intended is:

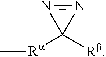

Typically, in an optionally substituted group or moiety:

(i) each hydrogen atom may optionally be replaced by a group independently selected from halo; —CN; —NO₂; —N₃; —R^{β}; —OH; —OR^{β}; —R^{α}-halo; —R^{α}—CN; —R^{α}—NO₂; —R^{α}—N₃; —R^{α}—R^{β}; —R^{α}—OH; —R^{α}—OR^{β}; —SH; —SR^{β}; —SOR^{β}; —SO₂H; —SO₂R^{β}; —SO₂NH₂; —SO₂NHR^{β}; —SO₂N(R^{β})₂; —R^{α}—SH; —R^{α}—SR^{β}; —R^{α}—SOR^{β}; —R^{α}—SO₂H; —R^{α}—SO₂R^{β}; —R^{α}—SO₂NH₂; —R^{α}—SO₂NHR^{β}; —R^{α}—SO₂N(R^{β})₂; —NH₂; —NHR^{β}; —N(R^{β})₂; —N(O)(R^{β})₂; —N⁺(R^{β})₃; —R^{α}—NH₂; —R^{α}—NHR^{β}; —R^{α}—N(R^{β})₂; —R^{α}—N(O)(R^{β})₂; —R^{α}—N⁺(R^{β})₃; —CHO; —COR^{β}; —COOH; —COOR^{β}; —OCOR^{β}; —R^{α}—CHO; —R^{α}—COR^{β}; —R^{α}—COOH; —R^{α}—COOR^{β}; —R^{α}—OCOR^{β}; —NH—CHO; —NR^{β}—CHO; —NH—COR^{β}; —NR—COR^{β}; —CONH₂; —CONHR^{β}; —CON(R^{β})₂; —R^{α}—NH—CHO; —R^{α}—NR^{β}—CHO; —R^{α}—NH—COR^{β}; —R^{α}—NR^{β}—COR^{β}; —R^{α}—CONH₂; —R^{α}—CONHR^{β}; —R^{α}—CON(R^{β})₂; —O—R^{α}—OH; —O—R^{α}—OR; —O—R^{α}—NH₂; —O—R^{α}—NH^{β}; —O—R^{α}—N(R^{β})₂; —O—R^{α}—N(O)(R^{β})₂; —O—R^{α}—N⁺(R^{β})₃; —NH—R^{α}—OH; —NH—R^{α}—OR^{β}; —NH—R^{α}—NH₂; —NH—R^{α}—NHR^{β}; —NH—R^{α}—N(R^{β})₂; —NH—R^{α}—N(O)(R^{β})₂; —NH—R^{α}—N⁺(R^{β})₃; —NR^{β}—R^{α}—OH; —NR^{β}—R^{α}—OR^{β}; —NR^{β}—R^{α}—NH₂; —NR^{β}—R^{α}—NHR^{β}; —NR^{β}—R^{α}—N(R^{β})₂; —NR^{β}—R^{α}—N(O)(R^{β})₂; —NR^{β}—R^{α}—N⁺(R^{β})₃; —N(O)R^{β}—R^{α}—OH; —N(O)R^{β}—R^{α}—OR; —N(O)R^{β}—R^{α}—NH₂; —N(O)R^{β}—R^{α}—NHR^{β}; —N(O)R^{β}—R^{α}—N(R^{β})₂; —N(O)R^{β}—R^{α}—N(O)(R^{β})₂; —N(O)R^{β}—R^{α}—N⁺(R^{β})₃; —N⁺(R^{β})₂—R^{α}—OH; —N⁺(R^{β})₂—R^{α}—OR^{β}; —N⁺(R^{β})₂—R^{α}—NH₂; —N⁺(R^{β})₂—R^{α}—NH^{β}; —N⁺(R^{β})₂—R^{α}—N(R^{β})₂; or —N⁺(R^{β})₂—R^{α}—N(O)(R^{β})₂; and/or

(ii) any two hydrogen atoms attached to the same carbon atom may optionally be replaced by a π-bonded substituent independently selected from oxo (═O), ═S, ═NH or ═NR^{β}; and/or

(iii) any two hydrogen atoms attached to the same or different atoms, within the same optionally substituted group or moiety, may optionally be replaced by a bridging substituent independently selected from —O—, —S—, —NH—, —N(R^{β})—, —N(O)(R^{β})—, —N⁺(R^{β})₂— or —R^{α}—
wherein each —R^{α}— is independently selected from an alkylene, alkenylene or alkynylene group, wherein the alkylene, alkenylene or alkynylene group contains from 1 to 6 atoms in its backbone, wherein one or more carbon atoms in the backbone of the alkylene, alkenylene or alkynylene group may optionally be replaced by one or more heteroatoms N, O or S, wherein one or more —CH₂— groups in the backbone of the alkylene, alkenylene or alkynylene group may optionally be replaced by one or more —N(O)(R^{β})— or —N⁺(R^{β})₂— groups, and wherein the alkylene, alkenylene or alkynylene group may optionally be substituted with one or more halo and/or —R^{β} groups; and
wherein each —R^{β} is independently selected from a C₁-C₆ alkyl, C₂-C₆ alkenyl, C₂-C₆ alkynyl or C₂-C₆ cyclic group, or wherein any two or three —R^{β} attached to the same nitrogen atom may, together with the nitrogen atom to which they are attached, form a C₂-C₇ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH, oxo (═O), or 4- to 6-membered heterocyclic group.

More typically, in an optionally substituted group or moiety:

(i) each hydrogen atom may optionally be replaced by a group independently selected from halo; —CN; —NO₂; —N₃; —R^{β}; —OH; —OR^{β}; —R^{α}-halo; —R^{α}—CN; —R^{α}—NO₂; —R^{α}—N₃; —R^{α}—R^{β}; —R^{α}—OH; —R^{α}—OR^{β}; —SH; —SR^{β}; —SOR^{β}; —SO₂H; —SO₂R^{β}; —SO₂NH₂; —SO₂NHR^{β}; —SO₂N(R^{β})₂; —R^{α}—SH; —R^{α}—SR^{β}; —R^{α}—SOR^{β}; —R^{α}—SO₂H; —R^{α}—SO₂R^{β}; —R^{α}—SO₂NH₂; —R^{α}—SO₂NHR^{β}; —R^{α}—SO₂N(R^{β})₂; —NH₂; —NHR^{β}; —N(R^{β})₂; —N⁺(R^{β})₃; —R^{α}—NH₂; —R^{α}—NHR^{β}; —R^{α}—N(R^{β})₂; —R^{α}—N⁺(R^{β})₃; —CHO; —COR^{β}; —COOH; —COOR^{β}; —OCOR^{β}; —R^{α}—CHO; —R^{α}—COR^{β}; —R^{α}—COOH; —R^{α}—COOR^{β}; or —R^{α}—OCOR^{β}; and/or

(ii) any two hydrogen atoms attached to the same carbon atom may optionally be replaced by a π-bonded substituent independently selected from oxo (═O), ═S, ═NH or ═NR^{β}; and/or

(iii) any two hydrogen atoms attached to the same or different atoms, within the same optionally substituted group or moiety, may optionally be replaced by a bridging substituent independently selected from —O—, —S—, —NH—, —N(R^{β})—, —N⁺(R^{β})₂— or —R^{α}—;
wherein each —R^{α}— is independently selected from an alkylene, alkenylene or alkynylene group, wherein the alkylene, alkenylene or alkynylene group contains from 1 to 6 atoms in its backbone, wherein one or more carbon atoms in the backbone of the alkylene, alkenylene or alkynylene group may optionally be replaced by one or more heteroatoms N, O or S, wherein a single —CH₂— group in the backbone of the alkylene, alkenylene or alkynylene group may optionally be replaced by a —N⁺(R^{β})₂— group, and wherein the alkylene, alkenylene or alkynylene group may optionally be substituted with one or more halo and/or —R^{β} groups; and
wherein each —R^{β} is independently selected from a C₁-C₆ alkyl, C₂-C₆ alkenyl, C₂-C₆ alkynyl or C₂-C₆ cyclic group, or wherein any two or three —R^{β} attached to the same nitrogen atom may, together with the nitrogen atom to which they are attached, form a C₂-C₇ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH, oxo (═O), or 4- to 6-membered heterocyclic group.

More typically, in an optionally substituted group or moiety:
(i) each hydrogen atom may optionally be replaced by a group independently selected from halo; —CN; —NO₂; —N₃; —R^{β}; —OH; —OR^{β}; —R^{α}-halo; —R^{α}—CN; —R^{α}—NO₂; —R^{α}—N₃; —R^{α}—R^{β}; —R^{α}—OH; —R^{α}—OR^{β}; —SH; —SR^{β}; —SOR^{β}; —SO₂H; —SO₂R^{β}; —SO₂NH₂; —SO₂NHR^{β}; —SO₂N(R^{β})₂; —R^{α}—SH; —R^{α}—SR^{β}; —R^{α}—SOR^{β}; —R^{α}—SO₂H; —R^{α}—SO₂R^{β}; —R^{α}—SO₂NH₂; —R^{α}—SO₂NHR^{β}; —R^{α}—SO₂N(R^{β})₂; —NH₂; —NHR^{β}; —N(R^{β})₂; —R^{α}—NH₂; —R^{α}—NHR^{β}; —R^{α}—N(R^{β})₂; —CHO; —COR^{β}; —COOH; —COOR^{β}; —OCOR^{β}; —R^{α}—CHO; —R^{α}—COR^{β}; —R^{α}—COOH; —R^{α}—COOR^{β}; or —R^{α}—OCOR^{β}; and/or

(ii) any two hydrogen atoms attached to the same carbon atom may optionally be replaced by a π-bonded substituent independently selected from oxo (═O), ═S, ═NH or ═NR^{β}; and/or

(iii) any two hydrogen atoms attached to the same or different atoms, within the same optionally substituted group or moiety, may optionally be replaced by a bridging substituent independently selected from —O—, —S—, —NH—, —N(R^{β})— or —R^{α}—;
wherein each —R^{α}— is independently selected from an alkylene, alkenylene or alkynylene group, wherein the alkylene, alkenylene or alkynylene group contains from 1 to 6 atoms in its backbone, wherein one or more carbon atoms in the backbone of the alkylene, alkenylene or alkynylene group may optionally be replaced by one or more heteroatoms N, O or S, and wherein the alkylene, alkenylene or alkynylene group may optionally be substituted with one or more halo and/or —R^{β} groups; and
wherein each —R^{β} is independently selected from a C₁-C₆ alkyl, C₂-C₆ alkenyl, C₂-C₆ alkynyl or C₂-C₆ cyclic group, or wherein any two —R^{β} attached to the same nitrogen atom may, together with the nitrogen atom to which they are attached, form a C₂-C₆ cyclic group, and wherein any —R^{β} may optionally be substituted with one or more C₁-C₄ alkyl, halo, —OH, or —O(C₁-C₄ alkyl) groups.

Typically a substituted group comprises 1, 2, 3 or 4 substituents, more typically 1, 2 or 3 substituents, more typically 1 or 2 substituents, and more typically 1 substituent. Unless stated otherwise, any divalent bridging substituent (e.g. —O—, —S—, —NH—, —N(R^{β})—, —N(O)(R^{β})—, —N⁺(R^{β})₂— or —R^{α}—) of an optionally substituted group or moiety (e.g. R¹) must only be attached to the specified group or moiety and may not be attached to a second group or moiety (e.g. R²), even if the second group or moiety can itself be optionally substituted.

The term “halo” includes fluoro, chloro, bromo and iodo.

Unless stated otherwise, where a group is prefixed by the term “halo”, such as a haloalkyl or halomethyl group, it is to be understood that the group in question is substituted with one or more halo groups independently selected from fluoro, chloro, bromo and iodo. Typically, the maximum number of halo substituents is limited only by the number of hydrogen atoms available for substitution on the corresponding group without the halo prefix. For example, a halomethyl group may contain one, two or three halo substituents. A haloethyl or halophenyl group may contain one, two, three, four or five halo substituents. Similarly, unless stated otherwise, where a group is prefixed by a specific halo group, it is to be understood that the group in question is substituted with one or more of the specific halo groups. For example, the term “fluoromethyl” refers to a methyl group substituted with one, two or three fluoro groups.

Unless stated otherwise, where a group is said to be “halo-substituted”, it is to be understood that the group in question is substituted with one or more halo groups independently selected from fluoro, chloro, bromo and iodo. Typically, the maximum number of halo substituents is limited only by the number of hydrogen atoms available for substitution on the group said to be halo-substituted. For example, a halo-substituted methyl group may contain one, two or three halo substituents. A halo-substituted ethyl or halo-substituted phenyl group may contain one, two, three, four or five halo substituents.

Unless stated otherwise, any reference to an element is to be considered a reference to all isotopes of that element. Thus, for example, unless stated otherwise any reference to hydrogen is considered to encompass all isotopes of hydrogen including deuterium and tritium.

Where reference is made to a hydrocarbyl or other group including one or more heteroatoms N, O or S in its carbon skeleton, or where reference is made to a carbon atom of a hydrocarbyl or other group being replaced by an N, O or S atom, what is intended is that:

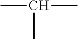

is replaced by

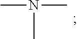

—CH₂— is replaced by —NH—, —O— or —S—;
—CH₃ is replaced by —NH₂, —OH or —SH;
—CH═ is replaced by —N═;
CH₂═ is replaced by NH═, O═ or S═; or
CH≡ is replaced by N≡;

provided that the resultant group comprises at least one carbon atom. For example, methoxy, dimethylamino and aminoethyl groups are considered to be hydrocarbyl groups including one or more heteroatoms N, O or S in their carbon skeleton.

Where reference is made to a —CH₂— group in the backbone of a hydrocarbyl or other group being replaced by a —N(O)(R^{β})— or —N⁺(R^{β})₂— group, what is intended is that:
—CH₂— is replaced by

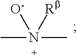

or
—CH₂— is replaced by

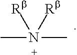

In the context of the present specification, unless otherwise stated, a Cₓ-C_{y} group is defined as a group containing from x to y carbon atoms. For example, a C₁-C₄ alkyl group is defined as an alkyl group containing from 1 to 4 carbon atoms. Optional substituents and moieties are not taken into account when calculating the total number of carbon atoms in the parent group substituted with the optional substituents and/or containing the optional moieties. For the avoidance of doubt, replacement heteroatoms, e.g. N, O or S, are not to be counted as carbon atoms when calculating the number of carbon atoms in a Cₓ-C_{y} group. For example, a morpholinyl group is to be considered a C₄ heterocyclic group, not a C₆ heterocyclic group.

Unless stated otherwise, any reference to a compound or group is to be considered a reference to all tautomers of that compound or group. Thus, for example, any reference a compound of formula (I) wherein Q¹ and Q² are both N and Q³ is NH is to be understood to encompass the tautomeric forms (a), (b) and (c) shown below:

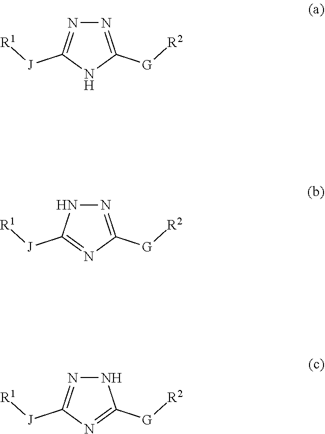

For the purposes of the present specification, where it is stated that a first atom or group is “directly attached” to a second atom or group it is to be understood that the first atom or group is covalently bonded to the second atom or group with no intervening atom(s) or group(s) being present. So, for example, for the group —(C═O)N(CH₃)₂, the carbon atom of each methyl group is directly attached to the nitrogen atom and the carbon atom of the carbonyl group is directly attached to the nitrogen atom, but the carbon atom of the carbonyl group is not directly attached to the carbon atom of either methyl group. For the avoidance of doubt, where it is stated that a group, such as R¹, R² or L, contains from x to y atoms other than hydrogen or halogen, it is to be understood that the group as a whole, including any optional substituents, contains from x to y atoms other than hydrogen or halogen. Such a group may contain any number of hydrogen or halogen atoms. Similarly, where it is stated that a group, such as R¹, R² or L, contains from x to y atoms other than hydrogen, it is to be understood that the group as a whole, including any optional substituents, contains from x to y atoms other than hydrogen. Such a group may contain any number of hydrogen atoms.

As stated, Q¹ and Q² are each independently selected from N or CR^{q}, provided that at least one of Q¹ and Q² is N. For example, Q¹ may be N where Q² is CR^{q}, or Q¹ may be CR^{q} where Q² is N, or both Q¹ and Q² may be N. Typically, Q¹ and Q² are both N.

Where Q¹ or Q² is CR^{q}, each R^{q} is independently selected from hydrogen or a halo, —OH, —NO₂, —NH₂, —N₃, —SH, —SO₂H, —SO₂NH₂, or a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton.

In one embodiment, each R^{q} where present is independently selected from hydrogen or a halo, —OH, —NH₂, —SH, or a saturated or unsaturated C₁-C₁₂ hydrocarbyl group, wherein the C₁-C₁₂ hydrocarbyl group may be straight-chained or branched, or be or include cyclic groups, wherein the C₁-C₁₂ hydrocarbyl group may optionally be substituted, and wherein the C₁-C₁₂ hydrocarbyl group may optionally include one, two or three heteroatoms N, O or S in its carbon skeleton. Where the hydrocarbyl group of R^{q} is optionally substituted, typically it is substituted with one or more groups independently selected from halo, —CN, —OH, —NH₂, —N(O)(R^{qp})₂, —N⁺(R^{qp})₃, oxo (═O) and ═NH, wherein each R^{qp} is independently selected from a C₁-C₄ alkyl, C₁-C₄ haloalkyl, C₃-C₄ cycloalkyl or C₃-C₄ halocycloalkyl group, or any two R^{qp} directly attached to the same nitrogen atom may together form a C₂-C₅ alkylene or C₂-C₅ haloalkylene group.

Typically, each R^{q} where present is independently selected from hydrogen or a halo or a saturated C₁-C₆ hydrocarbyl group, wherein the saturated C₁-C₆ hydrocarbyl group may be straight-chained or branched, or be or include cyclic groups, wherein the saturated C₁-C₆ hydrocarbyl group may optionally be substituted with one or more groups independently selected from halo, —CN, —OH, —NH₂, —N⁺(R^{qp})₃ and oxo (═O), wherein the saturated hydrocarbyl group may optionally include one or two heteroatoms N or O in its carbon skeleton, and wherein each R^{qp} is independently selected from a methyl or an ethyl group, wherein any methyl (Me) or ethyl (Et) group may optionally be substituted with one or more halo groups.

More typically, each R^{q} where present is independently selected from hydrogen or a fluoro, chloro, C₁-C₄ alkyl or C₃-C₄ cycloalkyl group, wherein the C₁-C₄ alkyl or C₃-C₄ cycloalkyl group may optionally be substituted with one or more fluoro and/or chloro groups. For example, each R^{q} where present may be independently selected from hydrogen or a fluoro, methyl, ethyl, n-propyl, isopropyl or cyclopropyl group, wherein any methyl, ethyl, n-propyl, isopropyl or cyclopropyl group may optionally be substituted with one or more fluoro groups.

Most typically, each R^{q} where present is hydrogen. In such an embodiment, Q¹ and Q² may each independently be selected from N or CH, provided that at least one of Q¹ and Q² is N.

As stated, Q³ is O, S or NR^{qq}, where each R^{qq} is independently selected from hydrogen or a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton.

In one embodiment, Q³ is O or NR^{qq}. More typically, Q³ is NR^{qq}.

In one embodiment, each R^{qq} where present is independently selected from hydrogen or a saturated or unsaturated C₁-C₁₂ hydrocarbyl group, wherein the C₁-C₁₂ hydrocarbyl group may be straight-chained or branched, or be or include cyclic groups, wherein the C₁-C₁₂ hydrocarbyl group may optionally be substituted, and wherein the C₁-C₁₂ hydrocarbyl group may optionally include one, two or three heteroatoms N, O or S in its carbon skeleton. Where the hydrocarbyl group of R^{qq} is optionally substituted, typically it is substituted with one or more groups independently selected from halo, —CN, —OH, —NH₂, —N(O)(R^{qg})₂, —N⁺(R^{qp})₃, oxo (═O) and ═NH, wherein each R^{qp} is independently selected from a C₁-C₄ alkyl, C₁-C₄ haloalkyl, C₃-C₄ cycloalkyl or C₃-C₄ halocycloalkyl group, or any two R^{qp} directly attached to the same nitrogen atom may together form a C₂-C₅ alkylene or C₂-C₅ haloalkylene group.

Typically, each R^{qq} where present is independently selected from hydrogen or a saturated C₁-C₆ hydrocarbyl group, wherein the saturated C₁-C₆ hydrocarbyl group may be straight-chained or branched, or be or include cyclic groups, wherein the saturated C₁-C₆ hydrocarbyl group may optionally be substituted with one or more groups independently selected from halo, —CN, —OH, —NH₂, —N⁺(R^{qp})₃ and oxo (═O), wherein the saturated hydrocarbyl group may optionally include one or two heteroatoms N or O in its carbon skeleton, and wherein each R^{qp} is independently selected from a methyl or an ethyl group, wherein any methyl (Me) or ethyl (Et) group may optionally be substituted with one or more halo groups.

More typically, each R^{qq} where present is independently selected from hydrogen or a C₁-C₄ alkyl or C₃-C₄ cycloalkyl group, wherein the C₁-C₄ alkyl or C₃-C₄ cycloalkyl group may optionally be substituted with one or more fluoro and/or chloro groups. For example, each R^{qq} where present may be independently selected from hydrogen or a methyl, ethyl, n-propyl, isopropyl or cyclopropyl group, wherein any methyl, ethyl, n-propyl, isopropyl or cyclopropyl group may optionally be substituted with one or more fluoro groups.

Most typically, each R^{qq} where present is hydrogen. In such an embodiment, Q³ may be selected from O, S or NH. Most typically, Q³ is NH.

As stated, J is a bond, —O—, —S—, —SO—, —SO₂—, —SO(═NR^{jj})—, —CO—, —C(═S)—, —C(R^{j})₂—, —C(═C(R^{jjj})₂)—, —C(═NR^{jj})—, —NR^{jj}—, —O—C(R^{j})₂—, —O—C(═C(R^{jjj})₂)—, —O—C(═NR^{jj})—, —S—C(R^{j})₂—, —S—C(═C(R^{jjj})₂)—, —S—C(═NR^{jj})—, —SO—C(R^{j})₂—, —SO—C(═C(R^{jjj})₂)—, —SO—C(═NR^{jj})—, —SO—NR^{jj}—, —SO₂—C(R^{j})₂—, —SO₂—C(═C(R^{jjj})₂)—, —SO₂—C(═NR^{jj})—, —SO₂—NR^{jj}—, —SO(═NR^{jj})—C(R^{j})₂—, —SO(═NR^{jj})—C(═C(R^{jjj})₂)—, —SO(═NR^{jj})—NR^{jj}—, —CO—C(R^{j})₂—, —CO—C(═C(R^{jjj})₂)—, —CO—C(═NR^{jj})—, —CO—NR^{jj}—, —C(═S)—C(R^{j})₂—, —C(═S)—C(═C(R^{jjj})₂)—, —C(R^{j})₂—O—, —C(R^{j})₂—S—, —C(R^{j})₂—SO—, —C(R^{j})₂—SO₂—, —C(R^{j})₂—SO(═NR^{jj})—, —C(R^{j})₂—CO—, —C(R^{j})₂—C(═S)—, —C(R^{j})₂—C(R^{j})₂—, —C(R^{j})₂—C(═C(R^{jjj})₂)—, —C(R^{j})₂—C(═NR^{jjj})—, —C(R^{j})₂—NR^{jj}—, —C(═C(R^{jjj})₂)—O—, —C(═C(R^{jjj})₂)—S—, —C(═C(R^{jjj})₂)—SO—, —C(═C(R^{jjj})₂)—SO₂—, —C(═C(R^{jjj})₂)—SO(═NR^{jj})—, —C(═C(R^{jjj})₂)—CO—, —C(═C(R^{jjj})₂)—C(═S)—, —C(═C(R^{jjj})₂)—C(R^{j})₂—, —C(═C(R^{jjj})₂)—C(═C(R^{jjj})₂)—, —C(═C(R^{jjj})₂)—C(═NR^{jj})—, —C(═C(R^{jjj})₂)—NR^{jj}—, —C(═NR^{jj})—O—, —C(═NR^{jj})—S—, —C(═NR^{jj})—SO—, —C(═NR^{jj})—SO₂—, —C(═NR^{jj})—CO—, —C(═NR^{jj})—C(R^{j})₂—, —C(═NR^{jjj})—C(═C(R^{jjj})₂)—, —C(═NR^{jj})—C(═NR^{jj})—, —C(═NR^{jj})—NR^{jj}—, —NR^{jj}—SO—, —NR^{jj}—SO₂—, —NR^{jj}—SO(═NR^{jj})—, —NR^{jj}—CO—, —NR^{jj}—C(R^{j})₂—, —NR^{jj}—C(═C(R^{jjj})₂)—, —NR^{jj}—C(═NR^{jj})—, —CR^{jjj}═CR^{jjj}—, —CR^{jjj}═N—, —N═CR^{jjj}— or —C≡C—.

In one embodiment, J is —S—, —SO—, —SO₂—, —SO(═NR^{jj})—, —S—C(R^{j})₂—, —SO—C(R^{j})₂—, —SO₂—C(R^{j})₂—, or —SO(═NR^{jj})—C(R^{j})₂—. Typically in such an embodiment, J is —S—, —SO—, —SO₂—, —SO(═NH)—, —S—CH₂—, —SO—CH₂—, —SO₂—CH₂—, or —SO(═NH)—CH₂—.

In another embodiment, J is —SO—, —SO₂—, —SO(═NR^{jj})—, —SO—C(R^{j})₂—, —SO₂—C(R^{j})₂—, or —SO(═NR^{jj})—C(R^{j})₂—. Typically in such an embodiment, J is —SO—, —SO₂—, —SO—C(R^{j})₂— or —SO₂—C(R^{j})₂—. More typically, J is —SO—, —SO₂—, —SO—CH₂— or —SO₂—CH₂—. Yet more typically, J is —SO—, —SO₂— or —SO₂—CH₂—.

In one embodiment, J is a bond, —O—, —S—, —SO—, —SO₂—, —SO(═NR^{jj})—, —CO—, —C(═S)—, —C(R^{j})₂—, —C(═C(R^{jjj})₂)—, —C(═NR^{jj})— or —NR^{jj}—.

In another embodiment, J is —O—, —S—, —SO—, —SO₂—, —SO(═NR^{jj})—, —CO—, —C(═S)—, —C(R^{j})₂—, —C(═C(R^{j})₂)—, —C(═NR^{jj})— or —NR^{jj}—.

In a further embodiment, J is —O—, —S—, —SO—, —SO₂—, —SO(═NR^{jj})—, —CO—, —C(═S)—, —C(R^{j})₂—, or —NR^{jj}—.

Typically, J is —S—, —SO—, —SO₂—, —SO(═NR^{jj})—, —CO— or —C(═S)—. More typically, J is —S—, —SO—, —SO₂— or —SO(═NR^{jj})—. Yet more typically, J is —SO—, —SO₂— or —SO(═NR^{jj})—. More typically still, J is —SO—, —SO₂— or —SO(═NH)—. Even more typically, J is —SO— or—SO₂—. Most typically, J is —SO₂—.

In one embodiment, each R^{j} where present is independently selected from hydrogen or a halo, —OH, —NO₂, —NH₂, —N₃, —SH, —SO₂H, —SO₂NH₂, or a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton.

In a further embodiment, each R^{j} where present is independently selected from hydrogen or a halo, —OH, —NH₂, —SH, or a saturated or unsaturated C₁-C₁₂ hydrocarbyl group, wherein the C₁-C₁₂ hydrocarbyl group may be straight-chained or branched, or be or include cyclic groups, wherein the C₁-C₁₂ hydrocarbyl group may optionally be substituted, and wherein the C₁-C₁₂ hydrocarbyl group may optionally include one, two or three heteroatoms N, O or S in its carbon skeleton. Where the hydrocarbyl group of R^{j} is optionally substituted, typically it is substituted with one or more groups independently selected from halo, —CN, —OH, —NH₂, —N(O)(R^{jp})₂, —N⁺(R^{jp})₃, oxo (═O) and ═NH, wherein each R^{jp} is independently selected from a C₁-C₄ alkyl, C₁-C₄ haloalkyl, C₃-C₄ cycloalkyl or C₃-C₄ halocycloalkyl group, or any two R^{jp} directly attached to the same nitrogen atom may together form a C₂-C₅ alkylene or C₂-C₅ haloalkylene group.

More typically, each R^{j} where present is independently selected from hydrogen or a halo, —OH, —NH₂, —CN, or a saturated C₁-C₆ hydrocarbyl group, wherein the saturated C₁-C₆ hydrocarbyl group may be straight-chained or branched, or be or include cyclic groups, wherein the saturated C₁-C₆ hydrocarbyl group may optionally be substituted with one or more groups independently selected from halo, —CN, —OH, —NH₂, —N⁺(R^{jp})₃ and oxo (═O), wherein the saturated hydrocarbyl group may optionally include one or two heteroatoms N or O in its carbon skeleton, and wherein each R^{jp} is independently selected from a methyl or an ethyl group, wherein any methyl (Me) or ethyl (Et) group may optionally be substituted with one or more halo groups.

More typically still, each R^{j} where present is independently selected from hydrogen or a halo, —OH, —NH₂, —CN, —R^{jx}, —OR^{jx}, —NHR^{jx} or —N(R^{jx})₂ group, wherein each R^{jx} is independently selected from a C₁-C₄ alkyl, C₁-C₄ haloalkyl, C₃-C₄ cycloalkyl or C₃-C₄ halocycloalkyl group, or any two R^{jx} directly attached to the same nitrogen atom may together form a C₂-C₅ alkylene or C₂-C₅ haloalkylene group. Typically, at least one R^{j} in any —C(R^{j})₂— group is selected from hydrogen or a halo, —CN or —R^{jx} group.

Yet more typically, a first R^{j} in any —C(R^{j})₂— group is independently selected from hydrogen or a fluoro, chloro, -Me or -Et group, and the second R^{j} in the —C(R^{j})₂— group is independently selected from hydrogen or a fluoro, chloro, —OH, —NH₂, -Me, -Et, —OMe,—OEt, —NHMe,—NHEt, —N(Me)₂, —N(Me)Et or —N(Et)₂ group, wherein any methyl (Me) or ethyl (Et) group may optionally be substituted with one or more fluoro and/or chloro groups.

Yet more typically still, each R^{j} where present is independently selected from hydrogen or a fluoro or methyl group, wherein the methyl group may optionally be substituted with one or more fluoro groups. Most typically, each R^{j} where present is hydrogen.

In one embodiment, each R^{j} where present is independently selected from hydrogen or a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton.

In a further embodiment, each R^{j} where present is independently selected from hydrogen or a saturated or unsaturated C₁-C₁₂ hydrocarbyl group, wherein the C₁-C₁₂ hydrocarbyl group may be straight-chained or branched, or be or include cyclic groups, wherein the C₁-C₁₂ hydrocarbyl group may optionally be substituted, and wherein the C₁-C₁₂ hydrocarbyl group may optionally include one, two or three heteroatoms N, O or S in its carbon skeleton. Where the hydrocarbyl group of R^{jj} is optionally substituted, typically it is substituted with one or more groups independently selected from halo, —CN, —OH, —NH₂, —N(O)(R^{jp})₂, —N⁺(R^{jp})₃, oxo (═O) and ═NH, wherein each R^{jp} is independently selected from a C₁-C₄ alkyl, C₁-C₄ haloalkyl, C₃-C₄ cycloalkyl or C₃-C₄ halocycloalkyl group, or any two R^{jp} directly attached to the same nitrogen atom may together form a C₂-C₅ alkylene or C₂-C₅ haloalkylene group.

More typically, each R^{jj} where present is independently selected from hydrogen, —CN or a saturated C₁-C₆ hydrocarbyl group, or from hydrogen or a saturated C₁-C₆ hydrocarbyl group, wherein in either scenario the saturated C₁-C₆ hydrocarbyl group may be straight-chained or branched, or be or include cyclic groups, wherein the saturated C₁-C₆ hydrocarbyl group may optionally be substituted with one or more groups independently selected from halo, —CN, —OH, —NH₂, —N⁺(R^{jp})₃ and oxo (═O), wherein the saturated C₁-C₆ hydrocarbyl group may optionally include one or two heteroatoms N or O in its carbon skeleton, and wherein each R^{jp} is independently selected from a methyl or an ethyl group, wherein any methyl (Me) or ethyl (Et) group may optionally be substituted with one or more halo groups.

More typically still, each R^{jj} where present is independently selected from hydrogen, —CN or a C₁-C₄ alkyl or C₃-C₄ cycloalkyl group, or from hydrogen or a C₁-C₄ alkyl or C₃-C₄ cycloalkyl group, wherein in either scenario the C₁-C₄ alkyl or C₃-C₄ cycloalkyl group may optionally be substituted with one or more fluoro and/or chloro groups. For example, each R^{jj} where present may be independently selected from hydrogen or a methyl, ethyl, n-propyl, isopropyl or cyclopropyl group, wherein any methyl, ethyl, n-propyl, isopropyl or cyclopropyl group may optionally be substituted with one or more fluoro groups.

Yet more typically, each R^{jj} where present is independently selected from hydrogen or a methyl group, wherein the methyl group may optionally be substituted with one or more fluoro groups. Most typically, each R^{jj} where present is hydrogen.

In one embodiment, each R^{jjj} where present is independently selected from hydrogen or a halo or a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton.

In a further embodiment, each R^{jjj} where present is independently selected from hydrogen or a halo or a saturated or unsaturated C₁-C₁₂ hydrocarbyl group, wherein the C₁-C₁₂ hydrocarbyl group may be straight-chained or branched, or be or include cyclic groups, wherein the C₁-C₁₂ hydrocarbyl group may optionally be substituted, and wherein the C₁-C₁₂ hydrocarbyl group may optionally include one, two or three heteroatoms N, O or S in its carbon skeleton. Where the hydrocarbyl group of R^{jjj} is optionally substituted, typically it is substituted with one or more groups independently selected from halo, —CN, —OH, —NH₂, —N(O)(R^{jp})₂, —N⁺(R^{jp})₃, oxo (═O) and ═NH, wherein each R^{jp} is independently selected from a C₁-C₄ alkyl, C₁-C₄ haloalkyl, C₃-C₄ cycloalkyl or C₃-C₄ halocycloalkyl group, or any two R^{jp} directly attached to the same nitrogen atom may together form a C₂-C₅ alkylene or C₂-C₅ haloalkylene group.

More typically, each R^{jjj} where present is independently selected from hydrogen or a halo or a saturated C₁-C₆ hydrocarbyl group, wherein the saturated C₁-C₆ hydrocarbyl group may be straight-chained or branched, or be or include cyclic groups, wherein the saturated C₁-C₆ hydrocarbyl group may optionally be substituted with one or more groups independently selected from halo, —CN, —OH, —NH₂, —N⁺(R^{jp})₃ and oxo (═O), wherein the saturated C₁-C₆ hydrocarbyl group may optionally include one or two heteroatoms N or O in its carbon skeleton, and wherein each R^{jp} is independently selected from a methyl or an ethyl group, wherein any methyl (Me) or ethyl (Et) group may optionally be substituted with one or more halo groups.

More typically still, each R^{jjj} where present is independently selected from hydrogen or a fluoro, chloro, C₁-C₄ alkyl or C₃-C₄ cycloalkyl group, wherein the C₁-C₄ alkyl or C₃-C₄ cycloalkyl group may optionally be substituted with one or more fluoro and/or chloro groups. For example, each R^{jjj} where present may be independently selected from hydrogen or a methyl, ethyl, n-propyl, isopropyl or cyclopropyl group, wherein any methyl, ethyl, n-propyl, isopropyl or cyclopropyl group may optionally be substituted with one or more fluoro groups.

Yet more typically, each R^{jjj} where present is independently selected from hydrogen or a fluoro or methyl group, wherein the methyl group may optionally be substituted with one or more fluoro groups.

In one embodiment, any two or three R^{j}, any two R^{jj}, any two or three R^{jjj}, or any two or three of R^{j}, R^{jj} and R^{jjj}, together with the atom or atoms to which they are attached, may form a saturated or unsaturated cyclic group, wherein the cyclic group may optionally be substituted.

In a further embodiment, any two or three R^{j}, any two R^{jj}, any two or three R^{jjj}, or any two or three of R^{j}, R^{jj} and R^{jjj}, together with the atom or atoms to which they are attached, may form a saturated or unsaturated 3- to 12-membered cyclic group, wherein the 3- to 12-membered cyclic group may optionally be substituted. Where the 3- to 12-membered cyclic group is optionally substituted, typically it is substituted with one or more groups independently selected from halo, —CN, —OH, —NO₂, —NH₂, oxo (═O), ═NH, —R^{jy}, —OR^{jy}, —NHR^{jy}, —N(R^{jy})₂, —N(O)(R^{jy})₂, —N⁺(R^{jy})₃ or ═NR^{jy}, wherein each R^{jy} is independently selected from a C₁-C₄ alkyl, C₁-C₄ haloalkyl, C₃-C₄ cycloalkyl or C₃-C₄ halocycloalkyl group, or any two R^{jy} directly attached to the same nitrogen atom may together form a C₂-C₅ alkylene or C₂-C₅ haloalkylene group.

More typically, any two R^{j}, any two R^{jj}, any two R^{jjj}, or any two of R^{j}, R^{jj} and R^{jjj}, together with the atom or atoms to which they are attached, may form a 3- to 7-membered saturated or unsaturated monocyclic group, wherein the monocyclic group may optionally be substituted with one or more groups independently selected from halo, —CN, —OH, —NO₂, —NH₂, oxo (═O), ═NH, -Me, -Et, —OMe,—OEt, —NHMe,—NHEt, —N(Me)₂, —N(Me)Et, —N(Et)₂, —N⁺(Me)₃, —N⁺(Me)₂Et, —N⁺(Et)₂Me or —N⁺(Et)₃, wherein any methyl (Me) or ethyl (Et) group may optionally be substituted with one or more halo groups.

In one embodiment, where J is or comprises —C(R^{j})₂—, any two R^{j} attached to the same carbon atom may, together with the carbon atom to which they are attached, form a 3- to 7-membered saturated or unsaturated monocyclic group, wherein the monocyclic group may optionally be substituted with one or more groups independently selected from halo, —CN, —OH, —NH₂, oxo (═O), ═NH, -Me, -Et, —OMe,—OEt, —NHMe,—NHEt, —N(Me)₂, —N(Me)Et, —N(Et)₂, —N⁺(Me)₃, —N⁺(Me)₂Et, —N⁺(Et)₂Me or —N⁺(Et)₃, wherein any methyl (Me) or ethyl (Et) group may optionally be substituted with one or more halo groups.

More typically, any two R^{j} attached to the same carbon atom may, together with the carbon atom to which they are attached, form a 3- or 4-membered cycloalkyl group, or form an oxetanyl group, wherein the 3- or 4-membered cycloalkyl group or the oxetanyl group may optionally be substituted with one or more fluoro and/or chloro groups. For example, in one embodiment any two R^{j} attached to the same carbon atom may, together with the carbon atom to which they are attached, form a cyclopropyl group, wherein the cyclopropyl group may optionally be substituted with one or more fluoro groups.

In one embodiment, where J is or comprises —C(R^{j})₂—, each R^{j} where present is independently selected from hydrogen or a fluoro, chloro, -Me or -Et group, wherein any methyl (Me) or ethyl (Et) group may optionally be substituted with one or more fluoro and/or chloro groups, or any two R^{j} attached to the same carbon atom may, together with the carbon atom to which they are attached, form a 3- or 4-membered cycloalkyl group, or form an oxetanyl group, wherein the 3- or 4-membered cycloalkyl group or the oxetanyl group may optionally be substituted with one or more fluoro and/or chloro groups. Typically in such an embodiment, each R^{j} where present is independently selected from hydrogen or a fluoro or methyl group, wherein the methyl group may optionally be substituted with one or more fluoro groups, or any two R^{j} attached to the same carbon atom may, together with the carbon atom to which they are attached, form a cyclopropyl group, wherein the cyclopropyl group may optionally be substituted with one or more fluoro groups.

As stated, G is a bond, —O—, —S—, —SO—, —SO₂—, —SO(═NR^{gg})—, —CO—, —C(═S)—, —C(R^{g})₂—, —C(═C(R^{ggg})₂)—, —C(═NR^{gg})—, —NR^{gg}—, —O—C(R^{g})₂—, —O—C(═C(R^{ggg})₂)—, —O—C(═NR^{gg})—, —S—C(R^{g})₂—, —S—C(═C(R^{ggg})₂)—, —S—C(═NR^{gg})—, —SO—C(R^{g})₂—, —SO—C(═C(R^{ggg})₂)—, —SO—C(═NR^{gg})—, —SO—NR^{gg}—, —SO₂—C(R^{g})₂—, —SO₂—C(═C(R^{ggg})₂)—, —SO₂—C(═NR^{gg})—, —SO₂—NR^{gg}—, —SO(═NR^{gg})—C(R^{g})₂—, —SO(═NR^{gg})—C(═C(R^{ggg})₂)—, —SO(═NR^{gg})—NR^{gg}—, —CO—C(R^{g})₂—, —CO—C(═C(R^{ggg})₂)—, —CO—C(═NR^{gg})—, —CO—NR^{gg}—, —C(═S)—C(R^{g})₂—, —C(═S)—C(═C(R^{ggg})₂)—, —C(R^{g})₂—O—, —C(R^{g})₂—S—, —C(R^{g})₂—SO—, —C(R^{g})₂—SO₂—, —C(R^{g})₂—SO(═NR^{gg})—, —C(R^{ggg})₂—CO—, —C(R^{g})₂—C(═S)—, —C(R^{g})₂—C(R^{g})₂—, —C(R^{g})₂—C(═C(R^{ggg})₂)—, —C(R^{g})₂—C(═NR^{gg})—, —C(R^{g})₂—NR^{gg}—, —C(═C(R^{ggg})₂)—O—, —C(═C(R^{ggg})₂)—S—, —C(═C(R^{ggg})₂)—SO—, —C(═C(R^{ggg})₂)—SO₂—, —C(═C(R^{ggg})₂)—SO(═NR^{gg})—, —C(═C(R^{ggg})₂)—CO—, —C(═C(R^{ggg})₂)—C(═S)—, —C(═C(R^{ggg})₂)—C(R^{g})₂—, —C(═C(R^{ggg})₂)—C(═C(R^{ggg})₂)—, —C(═C(R^{gg})₂)—C(═NR^{gg})—, —C(═C(R^{ggg})₂)—NR^{gg}—, —C(═NR^{gg})—O—, —C(═NR^{gg})—S—, —C(═NR^{gg})—SO—, —C(═NR^{gg})—SO₂—, —C(═NR^{gg})—CO—, —C(═NR^{gg})—C(R^{g})₂—, —C(═NR^{gg})—C(═C(R^{ggg})₂)—, —C(═NR^{gg})—C(═NR^{gg})—, —C(═NR^{gg})—NR^{gg}—, —NR^{gg}—SO—, —NR^{gg}—SO₂—, —NR^{gg}—SO(═NR^{gg})—, —NR^{gg}—CO—, —NR^{gg}—C(R^{g})₂—, —NR^{gg}—C(═C(R^{ggg})₂)—, —NR^{gg}—C(═NR^{gg})—, —CR^{ggg}═CR^{ggg}—, —CR^{ggg}═N—, —N═CR^{ggg}— or —C≡C—.

In one embodiment, G is a bond, —O—, —S—, —SO—, —SO₂—, —SO(═NR^{gg})—, —CO—, —C(═S)—, —C(R^{g})₂—, —C(═C(R^{ggg})₂)—, —C(═NR^{gg})— or —NR^{gg}—. Typically, G is a bond, —O—, —CO—, —C(═S)—, —C(R^{g})₂—, —C(═C(R^{ggg})₂)—, —C(═NR^{gg})— or —NR^{gg}—. More typically, G is a bond, —O—, —CO—, —C(R^{g})₂—, or —NR^{gg}—. More typically still, G is —O—, —C(R^{g})₂—, or —NR^{gg}—. Yet more typically, G is —O—, —CH₂—, or —NH—.

In one embodiment, G is —O— or —NR^{gg}—. Typically in such an embodiment, G is —O— or —NH—.

In another embodiment, G is —NR^{gg}—. Most typically in such an embodiment, G is —NH—.

In one embodiment, each R^{g} where present is independently selected from hydrogen or a halo, —OH, —NO₂, —NH₂, —N₃, —SH, —SO₂H, —SO₂NH₂, or a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton.

In a further embodiment, each R^{g} where present is independently selected from hydrogen or a halo, —OH, —NH₂, —SH, or a saturated or unsaturated C₁-C₁₂ hydrocarbyl group, wherein the C₁-C₁₂ hydrocarbyl group may be straight-chained or branched, or be or include cyclic groups, wherein the C₁-C₁₂ hydrocarbyl group may optionally be substituted, and wherein the C₁-C₁₂ hydrocarbyl group may optionally include one, two or three heteroatoms N, O or S in its carbon skeleton. Where the hydrocarbyl group of R^{g} is optionally substituted, typically it is substituted with one or more groups independently selected from halo, —CN, —OH, —NH₂, —N(O)(R^{gp})₂, —N⁺(R^{gp})₃, oxo (═O) and ═NH, wherein each R^{gp} is independently selected from a C₁-C₄ alkyl, C₁-C₄ haloalkyl, C₃-C₄ cycloalkyl or C₃-C₄ halocycloalkyl group, or any two R^{gp} directly attached to the same nitrogen atom may together form a C₂-C₅ alkylene or C₂-C₅ haloalkylene group.

More typically, each R^{g} where present is independently selected from hydrogen or a halo, —OH, —NH₂, —CN, or a saturated C₁-C₆ hydrocarbyl group, wherein the saturated C₁-C₆ hydrocarbyl group may be straight-chained or branched, or be or include cyclic groups, wherein the saturated C₁-C₆ hydrocarbyl group may optionally be substituted with one or more groups independently selected from halo, —CN, —OH, —NH₂, —N⁺(R^{gp})₃ and oxo (═O), wherein the saturated C₁-C₆ hydrocarbyl group may optionally include one or two heteroatoms N or O in its carbon skeleton, and wherein each R^{gp} is independently selected from a methyl or an ethyl group, wherein any methyl (Me) or ethyl (Et) group may optionally be substituted with one or more halo groups.

More typically still, each R^{g} where present is independently selected from hydrogen or a halo, —OH, —NH₂, —CN, —R^{gx}, —OR^{gx}, —NHR^{gx} or —N(R^{gx})₂ group, wherein each R^{gx} is independently selected from a C₁-C₄ alkyl, C₁-C₄ haloalkyl, C₃-C₄ cycloalkyl or C₃-C₄ halocycloalkyl group, or any two R^{gx} directly attached to the same nitrogen atom may together form a C₂-C₅ alkylene or C₂-C₅ haloalkylene group. Typically at least one R^{g} in any —C(R^{g})₂— group is selected from hydrogen or a halo, —CN or —R^{gx} group.

Yet more typically, a first R^{g} in any —C(R^{g})₂— group is independently selected from hydrogen or a fluoro, chloro, -Me or -Et group, and the second R^{g} in the —C(R^{g})₂— group is independently selected from hydrogen or a fluoro, chloro, —OH, —NH₂, -Me, -Et, —OMe,—OEt, —NHMe,—NHEt, —N(Me)₂, —N(Me)Et or —N(Et)₂ group, wherein any methyl (Me) or ethyl (Et) group may optionally be substituted with one or more fluoro and/or chloro groups.

Yet more typically still, each R^{g} where present is independently selected from hydrogen or a fluoro or methyl group, wherein the methyl group may optionally be substituted with one or more fluoro groups. Most typically, each R^{g} where present is hydrogen.

In one embodiment, each R^{gg} where present is independently selected from hydrogen or a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton.

In a further embodiment, each R^{gg} where present is independently selected from hydrogen or a saturated or unsaturated C₁-C₁₂ hydrocarbyl group, wherein the C₁-C₁₂ hydrocarbyl group may be straight-chained or branched, or be or include cyclic groups, wherein the C₁-C₁₂ hydrocarbyl group may optionally be substituted, and wherein the C₁-C₁₂ hydrocarbyl group may optionally include one, two or three heteroatoms N, O or S in its carbon skeleton. Where the hydrocarbyl group of R^{gg} is optionally substituted, typically it is substituted with one or more groups independently selected from halo, —CN, —OH, —NH₂, —N(O)(R^{gp})₂, —N⁺(R^{gp})₃, oxo (═O) and ═NH, wherein each R^{gp} is independently selected from a C₁-C₄ alkyl, C₁-C₄ haloalkyl, C₃-C₄ cycloalkyl or C₃-C₄ halocycloalkyl group, or any two R^{gp} directly attached to the same nitrogen atom may together form a C₂-C₅ alkylene or C₂-C₅ haloalkylene group.

More typically, each R^{gg} where present is independently selected from hydrogen or a saturated C₁-C₆ hydrocarbyl group, wherein the saturated C₁-C₆ hydrocarbyl group may be straight-chained or branched, or be or include cyclic groups, wherein the saturated C₁-C₆ hydrocarbyl group may optionally be substituted with one or more groups independently selected from halo, —CN, —OH, —NH₂, —N⁺(R^{gp})₃ and oxo (═O), wherein the saturated C₁-C₆ hydrocarbyl group may optionally include one or two heteroatoms N or O in its carbon skeleton, and wherein each R^{gp} is independently selected from a methyl or an ethyl group, wherein any methyl (Me) or ethyl (Et) group may optionally be substituted with one or more halo groups.

More typically still, each R^{gg} where present is independently selected from hydrogen or a C₁-C₄ alkyl or C₃-C₄ cycloalkyl group, wherein the C₁-C₄ alkyl or C₃-C₄ cycloalkyl group may optionally be substituted with one or more fluoro and/or chloro groups. For example, each R^{gg} where present may be independently selected from hydrogen or a methyl, ethyl, n-propyl, isopropyl or cyclopropyl group, wherein any methyl, ethyl, n-propyl, isopropyl or cyclopropyl group may optionally be substituted with one or more fluoro groups.

Yet more typically, each R^{gg} where present is independently selected from hydrogen or a methyl group, wherein the methyl group may optionally be substituted with one or more fluoro groups. Most typically, each R^{gg} where present is hydrogen.

In one embodiment, each R^{ggg} where present is independently selected from hydrogen or a halo or a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton.

In a further embodiment, each R^{ggg} where present is independently selected from hydrogen or a halo or a saturated or unsaturated C₁-C₁₂ hydrocarbyl group, wherein the C₁-C₁₂ hydrocarbyl group may be straight-chained or branched, or be or include cyclic groups, wherein the C₁-C₁₂ hydrocarbyl group may optionally be substituted, and wherein the C₁-C₁₂ hydrocarbyl group may optionally include one, two or three heteroatoms N, O or S in its carbon skeleton. Where the hydrocarbyl group of R^{ggg} is optionally substituted, typically it is substituted with one or more groups independently selected from halo, —CN, —OH, —NH₂, —N(O)(R^{gp})₂, —N⁺(R^{gp})₃, oxo (═O) and ═NH, wherein each R^{gp} is independently selected from a C₁-C₄ alkyl, C₁-C₄ haloalkyl, C₃-C₄ cycloalkyl or C₃-C₄ halocycloalkyl group, or any two RP directly attached to the same nitrogen atom may together form a C₂-C₅ alkylene or C₂-C₅ haloalkylene group.

More typically, each R^{ggg} where present is independently selected from hydrogen or a halo or a saturated C₁-C₆ hydrocarbyl group, wherein the saturated C₁-C₆ hydrocarbyl group may be straight-chained or branched, or be or include cyclic groups, wherein the saturated C₁-C₆ hydrocarbyl group may optionally be substituted with one or more groups independently selected from halo, —CN, —OH, —NH₂, —N⁺(R^{gp})₃ and oxo (═O), wherein the saturated C₁-C₆ hydrocarbyl group may optionally include one or two heteroatoms N or O in its carbon skeleton, and wherein each R^{gp} is independently selected from a methyl or an ethyl group, wherein any methyl (Me) or ethyl (Et) group may optionally be substituted with one or more halo groups.

More typically still, each R^{ggg} where present is independently selected from hydrogen or a fluoro, chloro, C₁-C₄ alkyl or C₃-C₄ cycloalkyl group, wherein the C₁-C₄ alkyl or C₃-C₄ cycloalkyl group may optionally be substituted with one or more fluoro and/or chloro groups. For example, each R^{ggg} where present may be independently selected from hydrogen or a methyl, ethyl, n-propyl, isopropyl or cyclopropyl group, wherein any methyl, ethyl, n-propyl, isopropyl or cyclopropyl group may optionally be substituted with one or more fluoro groups.

Yet more typically, each R^{ggg} where present is independently selected from hydrogen or a fluoro or methyl group, wherein the methyl group may optionally be substituted with one or more fluoro groups.

In one embodiment, any two or three R^{g}, any two R^{gg}, any two or three R^{ggg}, or any two or three of R^{g}, R^{gg} and R^{ggg}, together with the atom or atoms to which they are attached, may form a saturated or unsaturated cyclic group, wherein the cyclic group may optionally be substituted.

In a further embodiment, any two or three R^{g}, any two R^{gg}, any two or three R^{ggg}, or any two or three of R^{g}, R^{gg} and R^{ggg}, together with the atom or atoms to which they are attached, may form a saturated or unsaturated 3- to 12-membered cyclic group, wherein the 3- to 12-membered cyclic group may optionally be substituted. Where the 3- to 12-membered cyclic group is optionally substituted, typically it is substituted with one or more groups independently selected from halo, —CN, —OH, —NO₂, —NH₂, oxo (═O), ═NH, —R^{gy}, —OR^{gy}, —NHR^{gy}, —N(R^{gy})₂, —N(O)(R^{gy})₂, —N⁺(R^{gy})₃ or ═NR^{gy}, wherein each R^{gy} is independently selected from a C₁-C₄ alkyl, C₁-C₄ haloalkyl, C₃-C₄ cycloalkyl or C₃-C₄ halocycloalkyl group, or any two R^{gy} directly attached to the same nitrogen atom may together form a C₂-C₅ alkylene or C₂-C₅ haloalkylene group.

More typically, any two R^{g}, any two R^{gg}, any two R^{ggg}, or any two of R^{g}, R^{gg} and R^{ggg}, together with the atom or atoms to which they are attached, may form a 3- to 7-membered saturated or unsaturated monocyclic group, wherein the monocyclic group may optionally be substituted with one or more groups independently selected from halo, —CN, —OH, —NO₂, —NH₂, oxo (═O), ═NH, -Me, -Et, —OMe,—OEt, —NHMe,—NHEt, —N(Me)₂, —N(Me)Et, —N(Et)₂, —N⁺(Me)₃, —N⁺(Me)₂Et, —N⁺(Et)₂Me or —N⁺(Et)₃, wherein any methyl (Me) or ethyl (Et) group may optionally be substituted with one or more halo groups.

In one embodiment, where G is or comprises —C(R^{g})₂—, any two R^{g} attached to the same carbon atom may, together with the carbon atom to which they are attached, form a 3- to 7-membered saturated or unsaturated monocyclic group, wherein the monocyclic group may optionally be substituted with one or more groups independently selected from halo, —CN, —OH, —NH₂, oxo (═O), ═NH, -Me, -Et, —OMe,—OEt, —NHMe,—NHEt, —N(Me)₂, —N(Me)Et, —N(Et)₂, —N⁺(Me)₃, —N⁺(Me)₂Et, —N⁺(Et)₂Me or —N⁺(Et)₃, wherein any methyl (Me) or ethyl (Et) group may optionally be substituted with one or more halo groups.

More typically, any two R^{g} attached to the same carbon atom may, together with the carbon atom to which they are attached, form a 3- or 4-membered cycloalkyl group, or form an oxetanyl group, wherein the 3- or 4-membered cycloalkyl group or the oxetanyl group may optionally be substituted with one or more fluoro and/or chloro groups. For example, in one embodiment any two R^{g} attached to the same carbon atom may, together with the carbon atom to which they are attached, form a cyclopropyl group, wherein the cyclopropyl group may optionally be substituted with one or more fluoro groups.

In one embodiment, where G is or comprises —C(R^{g})₂—, each R^{g} where present is independently selected from hydrogen or a fluoro, chloro, -Me or -Et group, wherein any methyl (Me) or ethyl (Et) group may optionally be substituted with one or more fluoro and/or chloro groups, or any two R^{g} attached to the same carbon atom may, together with the carbon atom to which they are attached, form a 3- or 4-membered cycloalkyl group, or form an oxetanyl group, wherein the 3- or 4-membered cycloalkyl group or the oxetanyl group may optionally be substituted with one or more fluoro and/or chloro groups. Typically in such an embodiment, each R^{g} where present is independently selected from hydrogen or a fluoro or methyl group, wherein the methyl group may optionally be substituted with one or more fluoro groups, or any two R attached to the same carbon atom may, together with the carbon atom to which they are attached, form a cyclopropyl group, wherein the cyclopropyl group may optionally be substituted with one or more fluoro groups.

In one embodiment, the compound is a compound of formula (I) wherein:
Q¹ and Q² are each independently selected from N or CH, provided that at least one of Q¹ and Q² is N;
Q³ is O or NR^{qq};
J is —SO—, —SO₂— or —SO(═NH)—; and
G is —O—, —C(R^{g})₂—, or —NR^{gg}—;

wherein R¹, R², R^{qq}, R^{g} and R^{gg} are as defined herein.

In one aspect of such an embodiment:
R^{qq} is independently selected from hydrogen or a saturated C₁-C₆ hydrocarbyl group, wherein the saturated C₁-C₆ hydrocarbyl group may be straight-chained or branched, or be or include cyclic groups, wherein the saturated C₁-C₆ hydrocarbyl group may optionally be substituted with one or more groups independently selected from halo, —CN, —OH, —NH₂, —N⁺(R^{β})₃ and oxo (═O), wherein the saturated hydrocarbyl group may optionally include one or two heteroatoms N or O in its carbon skeleton, and wherein each R^{qp} is independently selected from a methyl or an ethyl group, wherein any methyl (Me) or ethyl (Et) group may optionally be substituted with one or more halo groups;
each R^{g} is independently selected from hydrogen or a halo, —OH, —NH₂, —CN, or a saturated C₁-C₆ hydrocarbyl group, wherein the saturated C₁-C₆ hydrocarbyl group may be straight-chained or branched, or be or include cyclic groups, wherein the saturated C₁-C₆ hydrocarbyl group may optionally be substituted with one or more groups independently selected from halo, —CN, —OH, —NH₂, —N⁺(R^{gp})₃ and oxo (═O), wherein the saturated C₁-C₆ hydrocarbyl group may optionally include one or two heteroatoms N or O in its carbon skeleton, and wherein each R^{gp} is independently selected from a methyl or an ethyl group, wherein any methyl (Me) or ethyl (Et) group may optionally be substituted with one or more halo groups, or any two R^{g} attached to the same carbon atom may, together with the carbon atom to which they are attached, form a 3- to 7-membered saturated or unsaturated monocyclic group, wherein the monocyclic group may optionally be substituted with one or more groups independently selected from halo, —CN, —OH, —NH₂, oxo (═O), ═NH, -Me, -Et, —OMe,—OEt, —NHMe,—NHEt, —N(Me)₂, —N(Me)Et, —N(Et)₂, —N⁺(Me)₃, —N⁺(Me)₂Et, —N⁺(Et)₂Me or —N⁺(Et)₃, wherein any methyl (Me) or ethyl (Et) group may optionally be substituted with one or more halo groups; and
R^{gg} is independently selected from hydrogen or a saturated C₁-C₆ hydrocarbyl group, wherein the saturated C₁-C₆ hydrocarbyl group may be straight-chained or branched, or be or include cyclic groups, wherein the saturated C₁-C₆ hydrocarbyl group may optionally be substituted with one or more groups independently selected from halo, —CN, —OH, —NH₂, —N⁺(R^{gp})₃ and oxo (═O), wherein the saturated C₁-C₆ hydrocarbyl group may optionally include one or two heteroatoms N or O in its carbon skeleton, and wherein each R^{gp} is independently selected from a methyl or an ethyl group, wherein any methyl (Me) or ethyl (Et) group may optionally be substituted with one or more halo groups.

In a further aspect of such an embodiment:
R^{qq} is selected from hydrogen or a C₁-C₄ alkyl or C₃-C₄ cycloalkyl group, wherein the C₁-C₄ alkyl or C₃-C₄ cycloalkyl group may optionally be substituted with one or more fluoro and/or chloro groups;
a first R^{g} is selected from hydrogen or a fluoro, chloro, -Me or -Et group, and a second R^{g} is selected from hydrogen or a fluoro, chloro, —OH, —NH₂, -Me, -Et, —OMe,—OEt, —NHMe,—NHEt, —N(Me)₂, —N(Me)Et or —N(Et)₂ group, wherein any methyl (Me) or ethyl (Et) group may optionally be substituted with one or more fluoro and/or chloro groups, or any two R^{g} attached to the same carbon atom may, together with the carbon atom to which they are attached, form a 3- or 4-membered cycloalkyl group, or form an oxetanyl group, wherein the 3- or 4-membered cycloalkyl group or the oxetanyl group may optionally be substituted with one or more fluoro and/or chloro groups; and
R^{gg} is selected from hydrogen or a C₁-C₄ alkyl or C₃-C₄ cycloalkyl group, wherein the C₁-C₄ alkyl or C₃-C₄ cycloalkyl group may optionally be substituted with one or more fluoro and/or chloro groups.

In a further embodiment, the compound is a compound of formula (Ia):

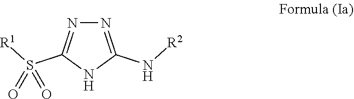

wherein R¹ and R² are as defined herein.

In another embodiment, the compound is a compound of formula (Ib):

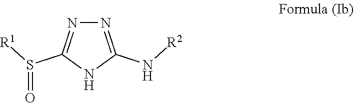

wherein R¹ and R² are as defined herein.

As stated, R¹ is a saturated or unsaturated hydrocarbyl group, such as a C₁-C₃₀ or C₂-C₂₀ or C₃-C₁₇ hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton.

In one embodiment, R¹ is a saturated or unsaturated C₁-C₂ or C₁-C₁₅ or C₁-C₁₂ hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton. In a further embodiment, R¹ is a saturated or unsaturated C₂-C₂₀ or C₂-C₁₅ or C₂-C₁₂ hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton.

In one embodiment, R¹ is a 3- to 12- or a 4- to 10-membered cyclic group, wherein the cyclic group may optionally be substituted. Typically the cyclic group is a cycloalkyl, cycloalkenyl, non-aromatic heterocyclic, aryl or heteroaryl group.

In one embodiment, R¹ is a 5- to 10-membered aryl or heteroaryl group, wherein the aryl or heteroaryl group may optionally be substituted. Typically in such an embodiment, the aryl or the heteroaryl group is monocyclic or bicyclic. More typically, R¹ is a phenyl or a 5- or 6-membered heteroaryl group, wherein the phenyl or the 5- or 6-membered heteroaryl group may optionally be substituted. For example, R¹ may be selected from the group consisting of phenyl, pyridinyl, pyrazinyl, pyrimidinyl, pyridazinyl, triazinyl, pyrrolyl, imidazolyl, pyrazolyl, triazolyl, tetrazolyl, furanyl, thiophenyl, oxazolyl, isoxazolyl, oxadiazolyl, thiazolyl, isothiazolyl and thiadiazolyl, all of which may optionally be substituted. More typically still, R¹ is a phenyl or a 5- or 6-membered heteroaryl group, wherein the 5- or 6-membered heteroaryl group contains at least one nitrogen atom, at least two carbon atoms and optionally an oxygen atom in its ring structure, and wherein the phenyl or the 5- or 6-membered heteroaryl group may optionally be substituted. For example, R¹ may be selected from the group consisting of phenyl, pyridinyl, pyrazinyl, pyrimidinyl, pyridazinyl, triazinyl, pyrrolyl, imidazolyl, pyrazolyl, triazolyl, oxazolyl, isoxazolyl and oxadiazolyl, all of which may optionally be substituted.

In another embodiment, R¹ is a 3- to 12-membered non-aromatic cyclic group, wherein the non-aromatic cyclic group may optionally be substituted. The non-aromatic cyclic group may be monocyclic, bicyclic (including bridged, fused and spiro), tricyclic or polycyclic, wherein the non-aromatic cyclic group may optionally be substituted. Typically the non-aromatic cyclic group is a monocyclic or a bicyclic group.

Where the non-aromatic cyclic group of R¹ is monocyclic, it may optionally be substituted with any monovalent substituent or any divalent π-bonded substituent, such as those defined herein, but may not be substituted with a divalent bridging substituent (e.g. —O—, —S—, —NH—, —N(R^{β})— or —R^{α}—) so as to form a bridged, fused or spiro substituent.

Where the non-aromatic cyclic group of R¹ is bicyclic, tricyclic or polycyclic, each ring in the bicyclic, tricyclic or polycyclic system, excluding any optional substituents, is non-aromatic. Typically, where the non-aromatic cyclic group of R¹ is bicyclic, tricyclic or polycyclic, the non-aromatic cyclic group of R¹ is a fused or spiro bicyclic, a fused or spiro tricyclic or a fused or spiro polycyclic system.

In one embodiment, R¹ is a 3- to 7-membered non-aromatic monocyclic group or a 7- to 12-membered non-aromatic bicyclic group, wherein the non-aromatic monocyclic group or the non-aromatic bicyclic group may optionally be substituted with one or more monovalent substituents and/or divalent π-bonded substituents.

Where R¹ is a 3- to 7-membered non-aromatic monocyclic group, typically R¹ is a 3-, 4-, 5- or 6-membered non-aromatic monocyclic group, more typically a 4-, 5- or 6-membered non-aromatic monocyclic group, and yet more typically a 5- or 6-membered non-aromatic monocyclic group, wherein the non-aromatic monocyclic group may optionally be substituted with one or more monovalent substituents and/or divalent 7-bonded substituents. Examples of monocyclic non-aromatic cyclic groups, which may be optionally substituted, include:

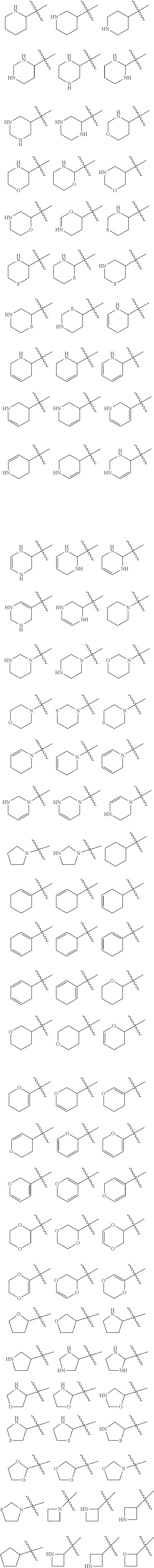

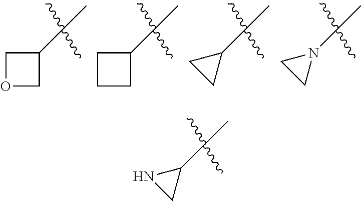

Where R¹ is a 7- to 12-membered non-aromatic bicyclic group, typically the non-aromatic bicyclic group is a fused bicyclic or a spiro bicyclic group, wherein the bicyclic group may optionally be substituted with one or more monovalent substituents and/or divalent π-bonded substituents. Typically, in such an embodiment, a first ring in the bicyclic system is a 4-, 5-, 6- or 7-membered ring and a second ring in the bicyclic system is a 3-, 4-, 5- or 6-membered ring. More typically, a first ring in the bicyclic system is a 4-, 5- or 6-membered ring and a second ring in the bicyclic system is a 4-, 5- or 6-membered ring.

The non-aromatic cyclic group of R¹ may be fully saturated or partially unsaturated. Accordingly, the non-aromatic cyclic group of R¹ may comprise one or more double bonds in the cyclic ring, provided the cyclic ring is non-aromatic. The non-aromatic cyclic group of R¹ does not have any tautomers that are aromatic.

In one embodiment, the non-aromatic cyclic group of R¹ is fully saturated. As will be understood, in such an embodiment all of the ring atoms of the non-aromatic cyclic group, when considered after any optional substitution, are sp³ hybridised. Thus, for example, in such an embodiment the non-aromatic cyclic group may not be substituted with a π-bonded substituent such as an oxo (═O) group.

In one embodiment, R¹ is a 3- to 7-membered fully saturated monocyclic group or a 7- to 12-membered fully saturated bicyclic group, wherein the fully saturated monocyclic group or the fully saturated bicyclic group may optionally be substituted with one or more monovalent substituents.

Where R¹ is a 3- to 7-membered fully saturated monocyclic group, typically R¹ is a 3-, 4-, 5- or 6-membered fully saturated monocyclic group, more typically a 4-, 5- or 6-membered fully saturated monocyclic group, and yet more typically a 5- or 6-membered fully saturated monocyclic group, wherein the fully saturated monocyclic group may optionally be substituted with one or more monovalent substituents.

Where R¹ is a 7- to 12-membered fully saturated bicyclic group, typically the fully saturated bicyclic group is a fused bicyclic or a spiro bicyclic group, wherein the bicyclic group may optionally be substituted with one or more monovalent substituents. Typically, in such an embodiment, a first ring in the bicyclic system is a 4-, 5-, 6- or 7-membered ring and a second ring in the bicyclic system is a 3-, 4-, 5- or 6-membered ring. More typically, a first ring in the bicyclic system is a 4-, 5- or 6-membered ring and a second ring in the bicyclic system is a 4-, 5- or 6-membered ring.

In one embodiment, R¹ is a 3- to 12-membered cycloalkyl or a cycloalkenyl group, wherein the cycloalkyl or cycloalkenyl group may optionally be substituted. Typically in such an embodiment, R¹ is a 3- to 12-membered cycloalkyl group, wherein the cycloalkyl group may optionally be substituted. More typically in such an embodiment, the R¹ is a 3- to 7-membered monocyclic cycloalkyl group, wherein the monocyclic cycloalkyl group may optionally be substituted. More typically still, R¹ is a cyclopropyl, cyclobutyl, cyclopentyl or cyclohexyl group, wherein the cyclopropyl, cyclobutyl, cyclopentyl or cyclohexyl group may optionally be substituted. Yet more typically, R¹ is a cyclobutyl, cyclopentyl or cyclohexyl group, wherein the cyclobutyl, cyclopentyl or cyclohexyl group may optionally be substituted.

In another embodiment, R¹ is a non-aromatic heterocyclic group, wherein the non-aromatic heterocyclic group may optionally be substituted. Typically in such an embodiment, R¹ is a fully saturated heterocyclic group, wherein the fully saturated heterocyclic group may optionally be substituted with one or more monovalent substituents. Typically, any non-aromatic heterocyclic group of R¹ contains one, two, three or four heteroatoms independently selected from oxygen, nitrogen and sulfur in its ring structure. More typically, any non-aromatic heterocyclic group of R¹ contains one, two or three heteroatoms independently selected from oxygen and nitrogen in its ring structure.

In one embodiment, R¹ is a 3- to 7-membered monocyclic non-aromatic heterocyclic group, wherein the monocyclic non-aromatic heterocyclic group may optionally be substituted with one or more monovalent substituents and/or divalent π-bonded substituents. Typically in such an embodiment, R¹ is a 4-, 5- or 6-membered fully saturated monocyclic heterocyclic group, wherein the 4-, 5- or 6-membered fully saturated monocyclic heterocyclic group contains one or two heteroatoms independently selected from oxygen and nitrogen in its ring structure, and wherein the fully saturated monocyclic heterocyclic group may optionally be substituted with one or more monovalent substituents. More typically, R¹ is selected from an oxetanyl, azetidinyl, pyrrolidinyl, tetrahydrofuranyl, pyrazolidinyl, imidazolidinyl, oxazolidinyl, isoxazolidinyl, dioxolanyl, piperidinyl, tetrahydropyranyl, piperazinyl, dioxanyl or morpholinyl group, any of which may optionally be substituted.

In another embodiment, R is a 7- to 12-membered bicyclic non-aromatic heterocyclic group, wherein the bicyclic non-aromatic heterocyclic group may optionally be substituted with one or more monovalent substituents and/or divalent π-bonded substituents. Typically, the 7- to 12-membered bicyclic non-aromatic heterocyclic group is a 7- to 12-membered fully saturated bicyclic group, wherein the bicyclic group may optionally be substituted with one or more monovalent substituents.

In one embodiment, R¹ is a 7- to 12-membered fused bicyclic or spiro bicyclic non-aromatic heterocyclic group, wherein the bicyclic group may optionally be substituted with one or more monovalent substituents and/or divalent π-bonded substituents. Typically, the 7- to 12-membered fused bicyclic or spiro bicyclic non-aromatic heterocyclic group is a 7- to 12-membered fully saturated bicyclic group, wherein the bicyclic group may optionally be substituted with one or more monovalent substituents.

Typically, where R¹ is a 7- to 12-membered fused bicyclic or spiro bicyclic non-aromatic heterocyclic group, a first ring in the bicyclic system is a 4-, 5-, 6- or 7-membered ring and a second ring in the bicyclic system is a 3-, 4-, 5- or 6-membered ring. More typically, a first ring in the bicyclic system is a 4-, 5- or 6-membered ring and a second ring in the bicyclic system is a 4-, 5- or 6-membered ring. Typically, the 7- to 12-membered bicyclic non-aromatic heterocyclic group contains one, two, three or four heteroatoms independently selected from oxygen and nitrogen in its ring structure. More typically, the 7- to 12-membered bicyclic non-aromatic heterocyclic group contains at least one nitrogen atom and one or two further heteroatoms independently selected from oxygen and nitrogen in its ring structure. Examples of such 7- to 12-membered bicyclic non-aromatic heterocyclic groups, which may be optionally substituted, include:

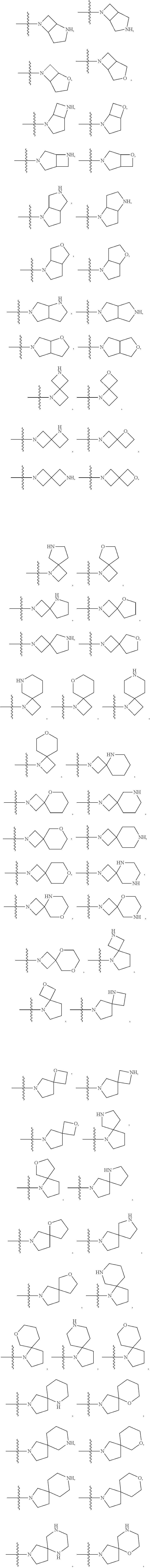

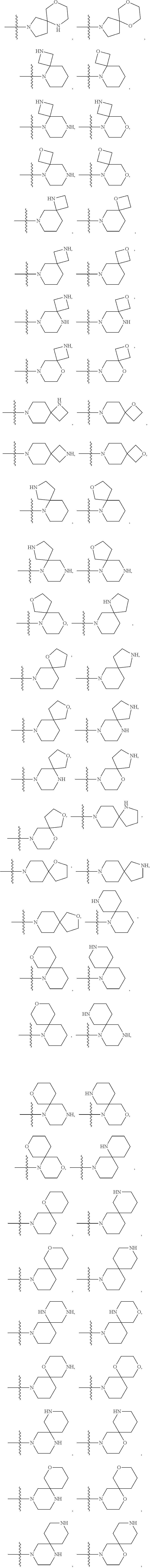

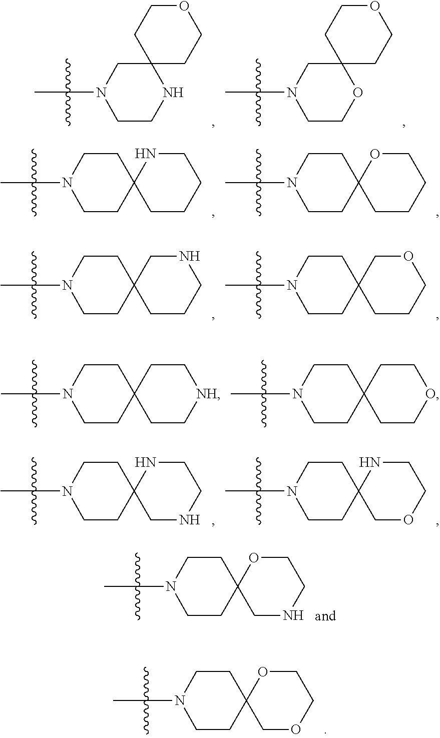

In another embodiment, R¹ is a partially aromatic bicyclic, tricyclic or polycyclic group, wherein at least one ring structure in the bicyclic, tricyclic or polycyclic group is non-aromatic and at least one ring structure is aromatic, and wherein the bicyclic, tricyclic or polycyclic group may optionally be substituted. In one variation, the ring of the bicyclic, tricyclic or polycyclic group of R¹ that is directly attached to J is aromatic, such that the bicyclic, tricyclic or polycyclic group may be seen as an aryl or heteroaryl group substituted with a saturated or partially unsaturated divalent bridging substituent so as to form a fused non-aromatic substituent. In another variation, the ring of the bicyclic, tricyclic or polycyclic group of R¹ that is directly attached to J is non-aromatic, such that the partially aromatic bicyclic, tricyclic or polycyclic group may be seen as a non-aromatic cyclic group substituted with an unsaturated divalent bridging substituent so as to form a fused aromatic substituent.

Where R¹ is a partially aromatic bicyclic, tricyclic or polycyclic group, any non-aromatic ring structure within such a group may be a non-aromatic hydrocarbyl ring structure or a non-aromatic heterocyclic ring structure. Similarly, any aromatic ring structure may be an aryl ring structure or a heteroaryl ring structure.

Typically, where R¹ is a partially aromatic bicyclic, tricyclic or polycyclic group, the bicyclic, tricyclic or polycyclic group is a fused bicyclic, a fused tricyclic or a fused polycyclic group, wherein at least one fused ring structure is aromatic and at least one fused ring structure is non-aromatic. In such a system it is to be understood that each ring in the fused bicyclic, fused tricyclic or fused polycyclic group, excluding any optional substituents, is fused to at least one other ring in the group. More typically, where R¹ is a partially aromatic bicyclic, tricyclic or polycyclic group, the bicyclic, tricyclic or polycyclic group is a fused bicyclic or a fused tricyclic group. Yet more typically, where R¹ is a partially aromatic bicyclic, tricyclic or polycyclic group, the bicyclic, tricyclic or polycyclic group is a fused bicyclic group.

In one embodiment, R¹, is a 8- to 10-membered fused bicyclic group, wherein a first ring in the fused bicyclic structure is a non-aromatic ring and a second ring in the fused bicyclic structure is an aromatic ring, and wherein the fused bicyclic group may optionally be substituted.

In another embodiment, R¹ is a 8- to 10-membered fused bicyclic group, wherein a first ring in the fused bicyclic structure is a non-aromatic heterocyclic ring and a second ring in the fused bicyclic structure is a heteroaryl ring, and wherein the fused bicyclic group may be optionally substituted.

Where R¹ is a partially aromatic bicyclic or tricyclic group, the partially aromatic bicyclic or tricyclic group may optionally be substituted with any monovalent substituent or any divalent π-bonded substituent, such as those defined herein, but may not be substituted with a divalent bridging substituent (e.g. —O—, —S—, —NH—, —N(R^{β})— or —R^{α}—) so as to form a bridged, fused or spiro substituent.

In one embodiment, R¹ is R¹⁰-L-, wherein:
L is —NH—, or an alkylene, alkenylene or alkynylene group, wherein one or more carbon atoms in the backbone of the alkylene, alkenylene or alkynylene group may optionally be replaced by one or more heteroatoms N, O or S, and wherein the alkylene, alkenylene or alkynylene group may optionally be substituted; and
R¹⁰ is a cyclic group, wherein the cyclic group may optionally be substituted.

For the avoidance of doubt, it is noted that it is a ring atom of the cyclic group of R¹⁰ that is directly attached to L, not any optional substituent.

R¹⁰ may be any 3- to 12-membered cyclic group, as described above in relation to R¹, wherein the cyclic group may optionally be substituted. Typically, R¹⁰ is monocyclic.

In one embodiment, R¹⁰ is a phenyl or a 5- or 6-membered heteroaryl group, wherein the phenyl or the 5- or 6-membered heteroaryl group may optionally be substituted.

For example, R¹⁰ may be selected from the group consisting of phenyl, pyridinyl, pyrazinyl, pyrimidinyl, pyridazinyl, triazinyl, pyrrolyl, imidazolyl, pyrazolyl, triazolyl, tetrazolyl, furanyl, thiophenyl, oxazolyl, isoxazolyl, oxadiazolyl, thiazolyl, isothiazolyl and thiadiazolyl, all of which may optionally be substituted. More typically, R¹⁰ is a phenyl or a 5- or 6-membered heteroaryl group, wherein the 5- or 6-membered heteroaryl group contains at least one nitrogen atom, at least two carbon atoms and optionally an oxygen atom in its ring structure, and wherein the phenyl or the 5- or 6-membered heteroaryl group may optionally be substituted. For example, R¹⁰ may be selected from the group consisting of phenyl, pyridinyl, pyrazinyl, pyrimidinyl, pyridazinyl, triazinyl, pyrrolyl, imidazolyl, pyrazolyl, triazolyl, oxazolyl, isoxazolyl and oxadiazolyl, all of which may optionally be substituted.

In another embodiment, R¹⁰ is a 3- to 7-membered non-aromatic monocyclic group, wherein the non-aromatic monocyclic group may optionally be substituted with one or more monovalent substituents and/or divalent π-bonded substituents. Typically in such an embodiment, R¹⁰ is a 3-, 4-, 5- or 6-membered non-aromatic monocyclic group, more typically a 4-, 5- or 6-membered non-aromatic monocyclic group, and yet more typically a 5- or 6-membered non-aromatic monocyclic group, wherein the non-aromatic monocyclic group may optionally be substituted with one or more monovalent substituents and/or divalent π-bonded substituents.

In one embodiment, R¹⁰ is a 3- to 7-membered fully saturated monocyclic group, wherein the fully saturated monocyclic group may optionally be substituted with one or more monovalent substituents. Typically in such an embodiment, R¹⁰ is a 3-, 4-, 5- or 6-membered fully saturated monocyclic group, more typically a 4-, 5- or 6-membered fully saturated monocyclic group, and yet more typically a 5- or 6-membered fully saturated monocyclic group, wherein the fully saturated monocyclic group may optionally be substituted with one or more monovalent substituents.

In one embodiment, R¹⁰ is a monocyclic 3- to 7-membered cycloalkyl or cycloalkenyl group, wherein the cycloalkyl or cycloalkenyl group may optionally be substituted. Typically in such an embodiment, R¹⁰ is a monocyclic 3- to 6-membered cycloalkyl group, wherein the cycloalkyl group may optionally be substituted. More typically in such an embodiment, R¹⁰ is a cyclopropyl, cyclobutyl, cyclopentyl or cyclohexyl group, wherein the cyclopropyl, cyclobutyl, cyclopentyl or cyclohexyl group may optionally be substituted.

In another embodiment, R¹⁰ is a 3- to 7-membered monocyclic non-aromatic heterocyclic group, wherein the monocyclic non-aromatic heterocyclic group may optionally be substituted with one or more monovalent substituents and/or divalent 7-bonded substituents. Typically in such an embodiment, R¹⁰ is a 4-, s- or 6-membered fully saturated monocyclic heterocyclic group, wherein the 4-, 5- or 6-membered fully saturated monocyclic heterocyclic group contains one or two heteroatoms independently selected from oxygen and nitrogen in its ring structure, and wherein the fully saturated monocyclic heterocyclic group may optionally be substituted with one or more monovalent substituents. More typically, R¹⁰ is selected from an oxetanyl, azetidinyl, pyrrolidinyl, tetrahydrofuranyl, pyrazolidinyl, imidazolidinyl, oxazolidinyl, isoxazolidinyl, dioxolanyl, piperidinyl, tetrahydropyranyl, piperazinyl, dioxanyl or morpholinyl group, any of which may optionally be substituted.

In one embodiment, L is —NH— or an alkylene or an alkenylene group, wherein the alkylene or alkenylene group may optionally include one or more heteroatoms N or O in its carbon skeleton, and wherein the alkylene or alkenylene group may optionally be substituted. Typically, L is —NH— or an alkylene group, wherein the alkylene group may optionally include one or two heteroatoms independently selected from oxygen and nitrogen in its carbon skeleton, wherein the alkylene group may optionally be substituted.

In one embodiment, L does not contain a carbonyl group. In one embodiment, L is unsubstituted.

Where L is substituted, typically it is substituted with one or more substituents independently selected from halo, —CN, —OH, —NH₂, oxo (═O) and ═NH. More typically, where L is substituted, it is substituted with one or more substituents independently selected from halo, —CN, —OH, —NH₂ and oxo (═O). Yet more typically, where L is substituted, it is substituted with one or more substituents independently selected from fluoro and oxo (═O).

Typically, L contains from 1 to 10 atoms other than hydrogen or halogen. More typically, L contains from 1 to 6 atoms other than hydrogen or halogen.

In one embodiment, L contains only atoms selected from the group consisting of carbon, hydrogen, nitrogen, oxygen and halogen atoms. Typically, L contains only atoms selected from the group consisting of carbon, hydrogen, nitrogen and halogen atoms.

Typically, L is —NH— or an alkylene group, wherein the alkylene group may optionally include a single nitrogen atom in its carbon skeleton, wherein the alkylene group may optionally be substituted with one or more fluoro groups, and wherein L contains from 1 to 6 atoms other than hydrogen or halogen.

More typically, L is —NH—, —NMe-, —CH₂—, —CH₂—CH₂—, —NH—CH₂—, —NMe-CH₂—, —CH₂—NH—, —CH₂—NMe-, —CH₂—CH₂—CH₂—, —NH—CH₂—CH₂—, —NMe-CH₂—CH₂—, —CH₂—NH—CH₂—, —CH₂—NMe-CH₂—, —CH₂—CH₂—NH—, —CH₂—CH₂—NMe-, —CH₂—CH₂—CH₂—CH₂—, —NH—CH₂—CH₂—CH₂—, —NMe-CH₂—CH₂—CH₂—, —CH₂—NH—CH₂—CH₂—, —CH₂—NMe-CH₂—CH₂—, —CH₂—CH₂—NH—CH₂—,—CH₂—CH₂—NMe-CH₂—,—CH₂—CH₂—CH₂—NH—, —CH₂—CH₂—CH₂—NMe-, —CH₂—CH₂—CH₂—CH₂—CH₂—, —NH—CH₂—CH₂—CH₂—CH₂—, —NMe-CH₂—CH₂—CH₂—CH₂—, —CH₂—NH—CH₂—CH₂—CH₂—, —CH₂—NMe-CH₂—CH₂—CH₂—, —CH₂—CH₂—NH—CH₂—CH₂—, —CH₂—CH₂—NMe-CH₂—CH₂—, —CH₂—CH₂—CH₂—NH—CH₂—, —CH₂—CH₂—CH₂—NMe-CH₂—, —CH₂—CH₂—CH₂—CH₂—NH— or —CH₂—CH₂—CH₂—CH₂—NMe-.

In one embodiment, R¹ is a phenyl, naphthyl, pyridinyl, pyridazinyl, pyrimidinyl, pyrazinyl, pyrrolyl, furanyl, thiophenyl, pyrazolyl, imidazolyl, triazolyl, oxazolyl, isoxazolyl, thiazolyl, isothiazolyl, cyclopropyl, cyclobutyl, cyclopentyl, cyclohexyl, cycloheptyl, azetinyl, azetidinyl, oxetanyl, thietanyl, pyrrolidinyl, tetrahydrofuranyl, tetrahydrothiophenyl, pyrazolidinyl, imidazolidinyl, 1,3-dioxolanyl, 1,2-oxathiolanyl, 1,3-oxathiolanyl, piperidinyl, tetrahydropyranyl, 1,4-dioxanyl or thianyl group, all of which may optionally be substituted. In one embodiment, R¹ is a pyrazolyl, imidazolyl, triazolyl, azetidinyl, pyrrolidinyl or piperidinyl group, all of which may optionally be substituted.

In another embodiment, R¹ is a C₁-C₁₅ alkyl, C₂-C₁₅ alkenyl or C₂-C₁₅ alkynyl group, all of which may optionally be substituted, and all of which may optionally include one or more (such as one, two or three) heteroatoms N, O or S in their carbon skeleton. R¹ may be a C₁-C₁₀ alkyl, C₂-C₁₀ alkenyl or C₂-C₁₀ alkynyl group, all of which may optionally be substituted, and all of which may optionally include one or more (such as one, two or three) heteroatoms N, O or S in its carbon skeleton. Typically in such an embodiment, R¹ is a C₁-C₁₀ alkyl group, wherein the C₁-C₁₀ alkyl group may optionally be substituted with one or more monovalent substituents and/or divalent π-bonded substituents, and wherein the C₁-C₁₀ alkyl group may optionally include one, two or three heteroatoms independently selected from oxygen and nitrogen in its carbon skeleton.

In one embodiment, R¹ is an optionally substituted C₁-C₅ alkyl or C₂-C₅ alkenyl group. For example, R¹ may be an optionally substituted methyl, ethyl, n-propyl or isopropyl group.

In another embodiment, R¹ is a C₂-C₈ alkyl group, wherein the C₂-C₈ alkyl group may optionally be substituted with one or more monovalent substituents and/or divalent 7-bonded substituents, and wherein the C₂-C₈ alkyl group includes one or two heteroatoms independently selected from oxygen and nitrogen in its carbon skeleton.

In another embodiment, R¹ is an optionally substituted phenyl or optionally substituted benzyl group.

In another embodiment, R¹ is a hydrocarbyl group, wherein the hydrocarbyl group may be straight-chained or branched, or be or include cyclic groups, wherein the hydrocarbyl group may optionally be substituted, and wherein the hydrocarbyl group includes one or more heteroatoms N or O in its carbon skeleton or is substituted with one or more groups comprising one or more heteroatoms N or O. Typically the hydrocarbyl group contains 1-15 carbon atoms and 1-4 nitrogen or oxygen atoms.

In another embodiment, R¹ is a saturated or unsaturated, optionally substituted, 4-, 5- or 6-membered heterocycle; or R¹ is an optionally substituted group selected from C₁-C₅ alkyl, C₂-C₅ alkenyl, C₂-C₅ alkynyl, C₃-C₆ cycloalkyl, phenyl or benzyl; or R¹ is a hydrocarbyl group, wherein the hydrocarbyl group may be straight-chained or branched, or be or include cyclic groups, wherein the hydrocarbyl group may optionally be substituted, and wherein the hydrocarbyl group includes one or more heteroatoms N or O in its carbon skeleton or is substituted with one or more groups comprising one or more heteroatoms N or O (typically the hydrocarbyl group contains 1-15 carbon atoms and 1-4 nitrogen or oxygen atoms).

In the above embodiments, R¹ may be substituted with one or more substituents independently selected from halo; —CN; —NO₂; —N₃; —R^{β}; —OH; —OR^{β}; —R^{α}-halo; —R^{α}—CN; —R^{α}—NO₂; —R^{α}—N₃; —R^{α}—R^{β}; —R^{α}—OH; —R^{α}—OR^{β}; —SH; —SR^{β}; —SOR^{β}; —SO₂H; —SO₂R^{β}; —SO₂NH₂; —SO₂NHR^{β}; —SO₂N(RP)₂; —R^{α}—SH; —R^{α}—SR^{β}; —R^{α}—SOR^{β}; —R^{α}—SO₂H; —R^{α}—SO₂R^{β}; —R^{α}—SO₂NH₂; —R^{α}—SO₂NHR^{β}; —R^{α}—SO₂N(RP)₂; —Si(R^{β})₃; —O—Si(R^{β})₃; —R^{α}—Si(R^{β})₃; —R^{α}—O—Si(R^{β})₃; —NH₂; —NHR^{β}; —N(R^{β})₂; —N(O)(R^{β})₂; —N⁺(R^{β})₃; —R^{α}—NH₂; —R^{α}—NHR^{β}; —R^{α}—N(R^{β})₂; —R^{α}—N(O)(R^{β})₂; —R^{α}—N⁺(R^{β})₃-CHO; —COR^{β}; —COOH; —COOR^{β}; —OCOR^{β}; —R^{α}—CHO; —R^{α}—COR^{β}; —R^{α}—COOH; —R^{α}—COOR^{β}; —R^{α}—OCOR^{β}; —C(═NH)R; —C(═NH)NH₂; —C(═NH)NHR^{β}; —C(═NH)N(R^{β})₂; —C(═NR^{β})R^{β}; —C(═NR^{β})NHR^{β}; —C(═NR^{β})N(R^{β})₂; —C(═NOH)R^{β}; —C(═NOR^{β})R^{β}; —C(N₂)R^{β}; —R^{α}—C(═NH)R^{β}; —R^{α}—C(═NH)NH₂; —R^{α}—C(═NH)NHR^{β}; —R^{α}—C(═NH)N(R^{β})₂; —R^{α}—C(═NR^{β})R^{β}; —R^{α}—C(═NR^{β})NHR^{β}; —R^{α}—C(═NR^{β})N(R^{β})₂; —R^{α}—C(═NOH)R^{β}; —R^{α}—C(═NOR^{β})R^{β}; —R^{α}—C(N₂)R^{β}; —NH—CHO; —NR^{β}—CHO; —NH—COR^{β}; —NR^{β}—COR^{β}; —CONH₂; —CONHR^{β}; —CON(R^{β})₂; —R^{α}—NH—CHO; —R^{α}—NR^{β}—CHO; —R^{α}—NH—COR^{β}; —R^{α}—NR^{β}—COR^{β}; —R^{α}—CONH₂; —R^{α}—CONHR^{β}; —R^{α}—CON(R^{β})₂; —O—R^{α}—OH; —O—R^{α}—OR^{β}; —O—R^{α}—NH₂; —O—R^{α}—NHR^{β}; —O—R^{α}—N(R^{β})₂; —O—R^{α}—N(O)(R^{β})₂; —O—R^{α}—N⁺(R^{β})₃; —NH—R^{α}—OH; —NH—R^{α}—OR^{β}; —NH—R^{α}—NH₂; —NH—R^{α}—NHR^{β}; —NH—R^{α}—N(R^{β})₂; —NH—R^{α}—N(O)(R^{β})₂; —NH—R^{α}—N⁺(R^{β})₃; —NR^{β}—R^{α}—OH; —NR^{β}—R^{α}—OR^{β}; —NR^{β}—R^{α}—NH₂; —NR^{β}—R^{α}—NHR^{β}; —NR^{β}—R^{α}—N(R^{β})₂; —NR^{β}—R^{α}—N(O)(R^{β})₂; —NR^{β}—R^{α}—N⁺(R^{β})₃; —N(O)R^{β}—R^{α}—OH; —N(O)R^{β}—R^{α}—OR^{β}; —N(O)R^{β}—R^{α}—NH₂; —N(O)R^{β}—R^{α}—NHR^{β}; —N(O)R^{β}—R^{α}—N(R^{β})₂; —N(O)R^{β}—R^{α}—N(O)(R^{β})₂; —N(O)R^{β}—R^{α}—N⁺(R^{β})₃; —N⁺(R^{β})₂—R^{α}—OH; —N⁺(R^{β})₂—R^{α}—OR^{β}; —N⁺(R^{β})₂—R^{α}—NH₂; —N⁺(R^{β})₂—R^{α}—NHR^{β}; —N⁺(R^{β})₂—R^{α}—N(R^{β})₂; or —N⁺(R^{β})₂—R^{α}—N(O)(R^{β})₂; a C₃-C₇ cycloalkyl group optionally substituted with one or more C₁-C₃ alkyl or C₁-C₃ haloalkyl groups; a C₅-C₇ cycloalkenyl group optionally substituted with one or more C₁-C₃ alkyl or C₁-C₃ haloalkyl groups; a 3- to 7-membered non-aromatic heterocyclic group optionally substituted with one or more C₁-C₆ alkyl or C₁-C₃ haloalkyl groups; oxo (═O); or a C₁-C₄ alkylene bridge;
wherein each —R^{α}— is independently selected from an alkylene, alkenylene or alkynylene group, wherein the alkylene, alkenylene or alkynylene group contains from 1 to 6 atoms in its backbone, wherein one or more carbon atoms in the backbone of the alkylene, alkenylene or alkynylene group may optionally be replaced by one or more heteroatoms N, O or S, wherein one or more —CH₂— groups in the backbone of the alkylene, alkenylene or alkynylene group may optionally be replaced by one or more —N(O)(R^{β})— or —N⁺(R^{β})₂— groups, and wherein the alkylene, alkenylene or alkynylene group may optionally be substituted with one or more halo and/or —R^{β} groups; and
wherein each —R^{β} is independently selected from a C₁-C₆ alkyl, C₂-C₆ alkenyl, C₂-C₆ alkynyl or C₂-C₆ cyclic group, or wherein any two or three —R^{β} attached to the same nitrogen atom may, together with the nitrogen atom to which they are attached, form a C₂-C₇ cyclic group, and wherein any —R^{β} may optionally be substituted with one or more C₁-C₄ alkyl, C₁-C₄ haloalkyl, C₃-C₇ cycloalkyl, C₃-C₇ halocycloalkyl, —O(C₁-C₄ alkyl), —O(C₁-C₄ haloalkyl), —O(C₃-C₇ cycloalkyl), —O(C₃-C₇ halocycloalkyl), —CO(C₁-C₄ alkyl), —CO(C₁-C₄ haloalkyl), —CO(C₃-C₇ cycloalkyl), —CO(C₃-C₇ halocycloalkyl), —COO(C₁-C₄ alkyl), —COO(C₁-C₄ haloalkyl), —COO(C₃-C₇ cycloalkyl), —COO(C₃-C₇ halocycloalkyl), halo, —OH, —NH₂, —CN, —C≡CH, oxo (═O), or 4- to 6-membered heterocyclic group.

Alternatively, R¹ may be substituted with one or more substituents independently selected from halo; —CN; —NO₂; —N₃; —R; —OH; —OR; —SH; —SR^{β}; —SOR^{β}; —SO₂H; —SO₂R^{β}; —SO₂NH₂; —SO₂NHR^{β}; —SO₂N(R^{β})₂; —R^{α}—SH; —R^{α}—SR^{β}; —R^{α}—SOR^{β}; —R^{α}—SO₂H; —R^{α}—SO₂R^{β}; —R^{α}—SO₂NH₂; —R^{α}—SO₂NHR^{β}; —R^{α}—SO₂N(R^{β})₂; —NH₂; —NHR^{β}; —N(R^{β})₂; —N(O)(R^{β})₂; —N⁺(R^{β})₃; —R^{α}—NH₂; —R^{α}—NHR^{β}; —R^{α}—N(R^{β})₂; —R^{α}—N(O)(R^{β})₂; —R^{α}—N⁺(R^{β})₃; —CHO; —COR^{β}; —COOH; —COOR^{β}; —OCOR^{β}; —R^{α}—CHO; —R^{α}—COR^{β}; —R^{α}—COOH; —R^{α}—COOR^{β}; —R^{α}—OCOR^{β}; —NH—CHO; —NR^{β}—CHO; —NH—COR^{β}; —NR^{β}—COR^{β}; —CONH₂; —CONHR^{β}; —CON(R^{β})₂; —R^{α}—NH—CHO; —R^{α}—NR^{β}—CHO; —R^{α}—NH—COR^{β}; —R^{α}—NR^{β}—COR^{β}; —R^{α}—CONH₂; —R^{α}—CONHR^{β}; —R^{α}—CON(R^{β})₂; —O—R^{α}—OH; —O—R^{α}—OR^{β}; —O—R^{α}—NH₂; —O—R^{α}—NHR^{β}; —O—R^{α}—N(R^{β})₂; —O—R^{α}—N(O)(R^{β})₂; —O—R^{α}—N⁺(R^{β})₃; —NH—R^{α}—OH; —NH—R^{α}—OR^{β}; —NH—R^{α}—NH₂; —NH—R^{α}—NHR^{β}; —NH—R^{α}—N(R^{β})₂; —NH—R^{α}—N(O)(R^{β})₂; —NH—R^{α}—N⁺(R^{β})₃; —NR^{β}—R^{α}—OH; —NR^{β}—R^{α}—OR^{β}; —NR^{β}—R^{α}—NH₂; —NR^{β}—R^{α}—NHR^{β}; —NR^{β}—R^{α}—N(R^{β})₂; —NR^{β}—R^{α}—N(O)(R^{β})₂; —NR^{β}—R^{α}—N⁺(R^{β})₃; —N(O)R^{β}—R^{α}—OH; —N(O)R^{β}—R^{α}—OR^{β}; —N(O)R^{β}—R^{α}—NH₂; —N(O)R^{β}—R^{α}—NHR^{β}; —N(O)R^{β}—R^{α}—N(R^{β})₂; —N(O)R^{β}—R^{α}—N(O)(R^{β})₂; —N(O)R^{β}—R^{α}—N⁺(R^{β})₃; —N⁺(R^{β})₂—R^{α}—OH; —N⁺(R^{β})₂—R^{α}—OR^{β}; —N⁺(R^{β})₂—R^{α}—NH₂; —N⁺(R^{β})₂—R^{α}—NHR^{β}; —N⁺(R^{β})₂—R^{α}—N(R^{β})₂; or —N⁺(R^{β})₂—R^{α}—N(O)(R^{β})₂; a C₃-C₇ cycloalkyl group optionally substituted with one or more C₁-C₃ alkyl or C₁-C₃ haloalkyl groups; a C₅-C₇ cycloalkenyl group optionally substituted with one or more C₁-C₃ alkyl or C₁-C₃ haloalkyl groups; a 3- to 7-membered non-aromatic heterocyclic group optionally substituted with one or more C₁-C₆ alkyl or C₁-C₃ haloalkyl groups; oxo (═O); or a C₁-C₄ alkylene bridge;
wherein each —R^{α}— is independently selected from an alkylene, alkenylene or alkynylene group, wherein the alkylene, alkenylene or alkynylene group contains from 1 to 6 atoms in its backbone, wherein one or more carbon atoms in the backbone of the alkylene, alkenylene or alkynylene group may optionally be replaced by one or more heteroatoms N, O or S, wherein one or more —CH₂— groups in the backbone of the alkylene, alkenylene or alkynylene group may optionally be replaced by one or more —N(O)(R^{β})— or —N⁺(R^{β})₂— groups, and wherein the alkylene, alkenylene or alkynylene group may optionally be substituted with one or more halo and/or —R^{β} groups; and
wherein each —R^{β} is independently selected from a C₁-C₆ alkyl, C₂-C₆ alkenyl, C₂-C₆ alkynyl or C₂-C₆ cyclic group, or wherein any two or three —R^{β} attached to the same nitrogen atom may, together with the nitrogen atom to which they are attached, form a C₂-C₇ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups.

Alternatively, R¹ may be substituted with one or more substituents independently selected from halo; —CN; —NO₂; —N₃; —R^{β}; —OH; —OR^{β}; —SH; —SR^{β}; —SOR^{β}; —SO₂H; —SO₂R^{β}; —SO₂NH₂; —SO₂NHR^{β}; —SO₂N(R^{β})₂; —R^{α}—SH; —R^{α}—SR^{β}; —R^{α}—SOR^{β}; —R^{α}—SO₂H; —R^{α}—SO₂R^{β}; —R^{α}—SO₂NH₂; —R^{α}—SO₂NHR^{β}; —R^{α}—SO₂N(R^{β})₂; —NH₂; —NHR^{β}; —N(R^{β})₂; —N(O)(R^{β})₂; —N⁺(R^{β})₃; —R^{α}—NH₂; —R^{α}—NHR^{β}; —R^{α}—N(R^{β})₂; —R^{α}—N(O)(R^{β})₂; —R^{α}—N⁺(R^{β})₃; —CHO; —COR^{β}; —COOH; —COOR^{β}; —OCOR^{β}; —R^{α}—CHO; —R^{α}—COR^{β}; —R^{α}—COOH; —R^{α}—COOR^{β}; —R^{α}—OCOR^{β}; —NH—CHO; —NR^{β}—CHO; —NH—COR^{β}; —NR^{β}—COR^{β}; —CONH₂; —CONHR^{β}; —CON(R^{β})₂; —R^{α}—NH—CHO; —R^{α}—NR^{β}—CHO; —R^{α}—NH—COR^{β}; —R^{α}—NR^{β}—COR^{β}; —R^{α}—CONH₂; —R^{α}—CONHR^{β}; —R^{α}—CON(R^{β})₂; oxo (═O); or a C₁-C₄ alkylene bridge;
wherein each —R^{α}— is independently selected from an alkylene, alkenylene or alkynylene group, wherein the alkylene, alkenylene or alkynylene group contains from 1 to 6 atoms in its backbone, wherein one or more carbon atoms in the backbone of the alkylene, alkenylene or alkynylene group may optionally be replaced by one or more heteroatoms N, O or S, wherein one or more —CH₂— groups in the backbone of the alkylene, alkenylene or alkynylene group may optionally be replaced by one or more —N(O)(R^{β})— or —N⁺(R^{β})₂— groups, and wherein the alkylene, alkenylene or alkynylene group may optionally be substituted with one or more halo and/or —R^{β} groups; and
wherein each —R^{β} is independently selected from a C₁-C₆ alkyl, C₂-C₆ alkenyl, C₂-C₆ alkynyl or C₂-C₆ cyclic group, or wherein any two or three —R^{β} attached to the same nitrogen atom may, together with the nitrogen atom to which they are attached, form a C₂-C₇ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups.

Alternatively, R¹ may be substituted with one or more substituents independently selected from halo; —CN; —NO₂; —N₃; —R^{β}; —OH; —OR^{β}; —SH; —SR^{β}; —SOR^{β}; —SO₂H; —SO₂R^{β}; —SO₂NH₂; —SO₂NHR^{β}; —SO₂N(R^{β})₂; —R^{α}—SH; —R^{α}—SR^{β}; —R^{α}—SOR^{β}; —R^{α}—SO₂H; —R^{α}—SO₂R^{β}; —R^{α}—SO₂NH₂; —R^{α}—SO₂NHR^{β}; —R^{α}—SO₂N(R^{β})₂; —NH₂; —NHR^{β}; —N(R^{β})₂; —N⁺(R^{β})₃; —R^{α}—NH₂; —R^{α}—NHR^{β}; —R^{α}—N(R^{β})₂; —R^{α}—N⁺(R^{β})₃; —CHO; —COR^{β}; —COOH; —COOR^{β}; —OCOR^{β}; —R^{α}—CHO; —R^{α}—COR^{β}; —R^{α}—COOH; —R^{α}—COOR^{β}; —R^{α}—OCOR^{β}; —CONH₂; —CONHR^{β}; —CON(R^{β})₂; oxo (═O); or a C₁-C₄ alkylene bridge;
wherein each —R^{α}— is independently selected from an alkylene, alkenylene or alkynylene group, wherein the alkylene, alkenylene or alkynylene group contains from 1 to 6 atoms in its backbone, wherein one or two carbon atoms in the backbone of the alkylene, alkenylene or alkynylene group may optionally be replaced by one or two heteroatoms N, O or S, wherein a single —CH₂— group in the backbone of the alkylene, alkenylene or alkynylene group may optionally be replaced by a —N⁺(R^{β})₂— group, and wherein the alkylene, alkenylene or alkynylene group may optionally be substituted with one or more halo and/or —R^{β} groups; and
wherein each —R^{β} is independently selected from a C₁-C₆ alkyl, C₂-C₆ alkenyl, C₂-C₆ alkynyl or C₂-C₆ cyclic group, or wherein any two or three —R^{β} attached to the same nitrogen atom may, together with the nitrogen atom to which they are attached, form a C₂-C₇ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups.

Alternatively still, R¹ may be substituted with one, two or three substituents independently selected from halo; —CN; —N₃; —R^{β}; —OH; —OR^{β}; —SO₂R^{β}; —NH₂; —NHR^{β}; —N(R^{β})₂; —N(O)(R^{β})₂; —N⁺(R^{β})₃; —R^{α}—NH₂; —R^{α}—NHR^{β}; —R^{α}—N(R^{β})₂; —R^{α}—N(O)(R^{β})₂; —R^{α}—N⁺(R^{β})₃; —COR^{β}; —COOR^{β}; —OCOR^{β}; —R^{α}—COR^{β}; —R^{α}—COOR^{β}; —R^{α}—OCOR^{β}; —CONH₂; —CONHR^{β}; —CON(R^{β})₂; or oxo (═O);
wherein each —R^{α}— is independently selected from a C₁-C₆ alkylene group, wherein one or two carbon atoms in the backbone of the alkylene group may optionally be replaced by one or two heteroatoms N, O or S, and wherein the alkylene group may optionally be substituted with one or two halo and/or —R^{β} groups; and
wherein each —R^{β} is independently selected from a C₁-C₆ alkyl, C₂-C₆ alkenyl, C₂-C₆ alkynyl or C₂-C₆ cyclic group, or wherein any two or three —R^{β} attached to the same nitrogen atom may, together with the nitrogen atom to which they are attached, form a C₂-C₇ cyclic group, and wherein any —R^{β} may optionally be substituted with one, two or three C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups.

Typically, where R¹ is substituted, R¹ is substituted with one or more halo groups and/or one, two or three non-halo substituents. More typically, where R¹ is substituted, R¹ is substituted with one or more fluoro groups and/or one or two non-halo substituents.

In one embodiment, R¹ may be optionally substituted with one or more halo groups, and/or with one, two or three substituents independently selected from C₁-C₅ alkyl, C₁-C₅ haloalkyl, C₃-C₆ cycloalkyl, C₃-C₆ halocycloalkyl, C₂-C₅ alkenyl, C₂-C₅ haloalkenyl, C₅-C₆ cycloalkenyl, C₅-C₆ halocycloalkenyl, C₂-C₅ alkynyl, C₂-C₅ haloalkynyl, phenyl, halophenyl, 5- or 6-membered heteroaryl (optionally halo substituted), —R¹—CN, —R¹¹—N₃, —R¹¹—NO₂, —R¹¹—N(R¹²)₂, —R¹¹—OR¹², —R¹¹—COR¹², —R¹¹—COOR¹², —R¹¹—CON(R¹²)₂, —R¹¹—SO₂R¹², R¹¹—SO₂N(R¹²)₂, oxo (═O),

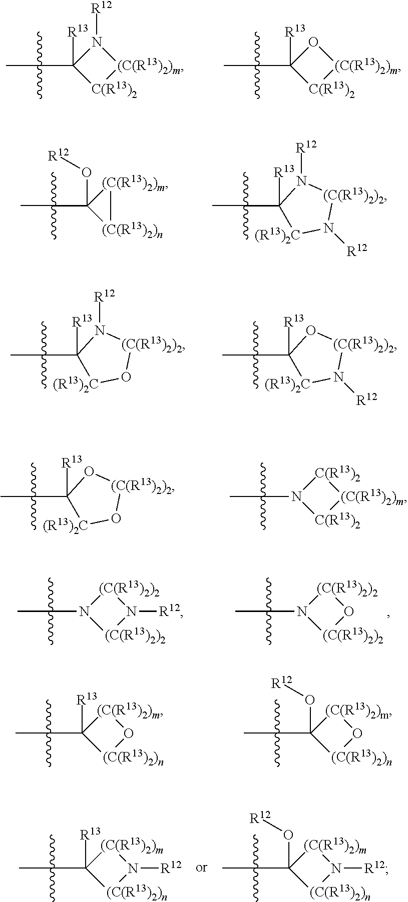

wherein R¹¹ is independently selected from a bond, C₁-C₄ alkylene, C₁-C₄ haloalkylene, C₃-C₄ cycloalkylene or C₃-C₄ halocycloalkylene; each R¹² is independently selected from hydrogen, C₁-C₅ alkyl, C₁-C₅ haloalkyl, C₃-C₅ cycloalkyl or C₃-C₅ halocycloalkyl, or any two R¹² attached to the same nitrogen atom may together form a C₂-C₅ alkylene or C₂-C₅ haloalkylene group; each R¹³ is independently selected from hydrogen or halo; m is 1, 2 or 3; and n is 1, 2 or 3. Typically, m+n 5.

In another embodiment, R¹ may be optionally substituted with one or more halo groups, and/or with one, two or three substituents independently selected from C₁-C₅ alkyl, C₁-C₅ haloalkyl, C₃-C₆ cycloalkyl, C₃-C₆ halocycloalkyl, C₂-C₅ alkenyl, C₂-C₅ haloalkenyl, C₅-C₆ cycloalkenyl, C₅-C₆ halocycloalkenyl, C₂-C₅ alkynyl, C₂-C₅ haloalkynyl, phenyl, halophenyl, 5- or 6-membered heteroaryl (optionally halo substituted), —R¹¹—CN, —R¹¹—N₃, —R¹¹—NO₂)₂, —R¹¹—N(R¹²)₂, —R¹¹—OR¹², —R¹¹—COR¹², —R¹¹—COOR¹², —R¹¹—CON(R¹²)₂, —R¹¹—SO₂R¹², —R¹¹—SO₂N(R¹²)₂, oxo (═O)

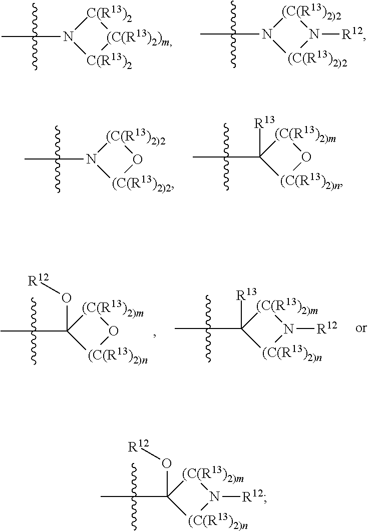

wherein R¹¹ is independently selected from a bond, C₁-C₄ alkylene, C₁-C₄ haloalkylene, C₃-C₄ cycloalkylene or C₃-C₄ halocycloalkylene; each R¹² is independently selected from hydrogen, C₁-C₅ alkyl, C₁-C₅ haloalkyl, C₃-C₅ cycloalkyl or C₃-C₅ halocycloalkyl, or any two R¹² attached to the same nitrogen atom may together form a C₂-C₅ alkylene or C₂-C₅ haloalkylene group; each R¹³ is independently selected from hydrogen or halo; m is 1, 2 or 3; and n is 1, 2 or 3. Typically, m+n≤5.

Typically in such an embodiment, R¹ may be optionally substituted with one or more halo groups, and/or with one or two substituents independently selected from C₁-C₅ alkyl, C₁-C₅ haloalkyl, C₃-C₆ cycloalkyl, C₃-C₆ halocycloalkyl, phenyl, halophenyl, 5- or 6-membered heteroaryl (optionally halo substituted), —R¹¹—CN, —R¹¹—N(R¹²)₂, —R¹¹—OR¹², —R¹¹—COR¹², —R¹¹—COOR¹², —R¹¹—CON(R¹²)₂, oxo (═O),

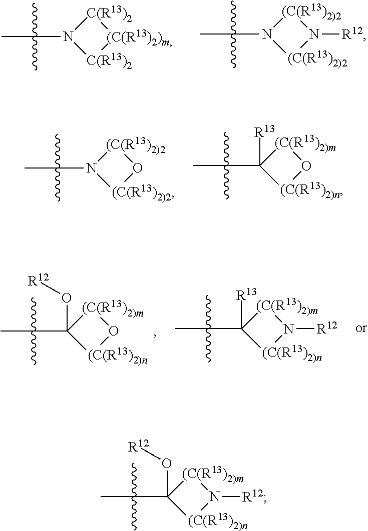

wherein R¹¹ is independently selected from a bond, C₁-C₄ alkylene or C₁-C₄ haloalkylene; each R¹² is independently selected from hydrogen, C₁-C₅ alkyl, C₁-C₅ haloalkyl, C₃-C₅ cycloalkyl or C₃-C₅ halocycloalkyl, or any two R¹² attached to the same nitrogen atom may together form a C₂-C₅ alkylene or C₂-C₅ haloalkylene group; each R¹³ is independently selected from hydrogen or halo; m is 1 or 2; and n is 1 or 2.

In another embodiment, R¹ may be optionally substituted with one or more halo groups, and/or with one, two or three substituents independently selected from C₁-C₅ alkyl, C₁-C₅ haloalkyl, C₃-C₆ cycloalkyl, C₃-C₆ halocycloalkyl, C₂-C₅ alkenyl, C₂-C₅ haloalkenyl, C₅-C₆ cycloalkenyl, C₅-C₆ halocycloalkenyl, C₂-C₅ alkynyl, C₂-C₅ haloalkynyl, phenyl, halophenyl, 5- or 6-membered heteroaryl (optionally halo substituted), —R¹¹—CN, —R¹¹—N₃, —R¹¹—NO₂, —R¹¹—N(R¹²)₂, —R¹¹—OR¹², —R¹¹—COR¹², —R¹¹—COOR¹², —R¹¹—CON(R¹²)₂, —R¹¹SO₂R¹², oxo (═O),

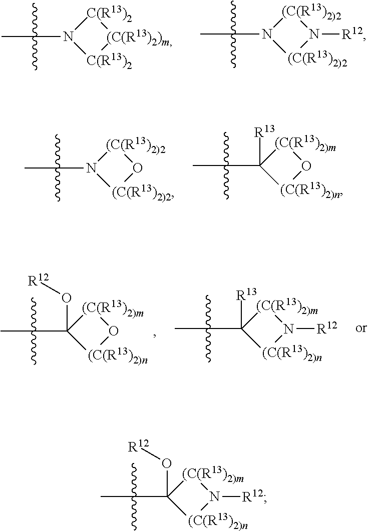

wherein R¹¹ is independently selected from a bond, C₁-C₄ alkylene or C₁-C₄ haloalkylene; each R¹² is independently selected from hydrogen, C₁-C₅ alkyl, C₁-C₅ haloalkyl, C₃-C₅ cycloalkyl or C₃-C₅ halocycloalkyl, or any two R¹² attached to the same nitrogen atom may together form a C₂-C₅ alkylene or C₂-C₅ haloalkylene group; each R¹³ is independently selected from hydrogen or halo; m is 1, 2 or 3; and n is 1, 2 or 3. Typically, m+n≤5.

In yet another embodiment, R¹ may be optionally substituted with one, two or three substituents independently selected from halo, C₁-C₅ alkyl, C₁-C₅ haloalkyl, C₃-C₆ cycloalkyl, C₂-C₅ alkenyl, C₂-C₅ haloalkenyl, C₂-C₅ alkynyl, C₂-C₅ haloalkynyl, —R—CN, —R¹¹—N₃, —R¹¹—NO₂, —R¹¹—N(R¹²)₂, —R¹¹—OR¹², —R¹¹—COR¹², —R¹¹—COOR¹², —R¹¹—CON(R¹²)₂, —R¹¹—SO₂R¹², oxo (═O),

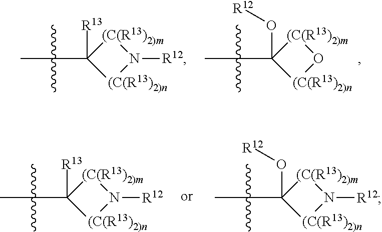

wherein R¹¹ is independently selected from a bond, C₁-C₃ alkylene or C₁-C₃ haloalkylene; each R¹² is independently selected from hydrogen, C₁-C₅ alkyl, C₁-C₅ haloalkyl, C₃-C₅ cycloalkyl or C₃-C₅ halocycloalkyl, or any two R¹² attached to the same nitrogen atom may together form a C₂-C₅ alkylene or C₂-C₅ haloalkylene group; each R¹³ is independently selected from hydrogen or halo; m is 1, 2 or 3; and n is 1, 2 or 3. Typically, m+n≤4.

In the above embodiments, where it is stated that R¹ may be (optionally) substituted, it will be understood that any component of R¹ that is stated to be (optionally) substituted, such as R¹⁰ or any (optionally) substituted hydrocarbyl, heteroatom-containing hydrocarbyl, cyclic, aryl, heteroaryl, non-aromatic cyclic, partially aromatic cyclic, alkyl, alkenyl or alkynyl group of R¹, or R¹⁰, may be (optionally) substituted with such substituents.

In one aspect of any of the above embodiments, R¹ contains from 1 to 30 atoms other than hydrogen or halogen. More typically, R¹ contains from 1 to 25 atoms other than hydrogen or halogen. More typically, R¹ contains from 1 to 20, or from 2 to 20 atoms other than hydrogen or halogen. More typically, R¹ contains from 1 to 17, or from 2 to 17, or from 4 to 17 atoms other than hydrogen or halogen. Yet more typically, R¹ contains from 1 to 15, or from 2 to 15, or from 4 to 15 atoms other than hydrogen or halogen.

In one aspect of any of the above embodiments, a carbon or nitrogen atom of R¹ is directly attached to a sulfur atom of J. In other words, R¹ is linked to J via a carbon-sulfur or a nitrogen-sulfur bond. In one embodiment, a carbon atom of R¹ is directly attached to a sulfur atom of J. In another embodiment, a nitrogen atom of R¹ is directly attached to a sulfur atom of J.

In one aspect of any of the above embodiments, R¹ contains only atoms selected from the group consisting of carbon, hydrogen, nitrogen, oxygen and halogen atoms. Typically, R¹ contains only atoms selected from the group consisting of carbon, hydrogen, nitrogen, oxygen and fluorine atoms.

As stated, R² is a cyclic group substituted at the α-position, wherein R² may optionally be further substituted. For the avoidance of doubt, it is noted that it is a ring atom of the cyclic group of R² that is directly attached to the group G, not any substituent.

In one embodiment of the first aspect of the invention, R² is an aryl or a heteroaryl group, wherein the aryl or the heteroaryl group is substituted at the α-position, and wherein R² may optionally be further substituted. Typically, R² is a phenyl or a 5- or 6-membered heteroaryl group, wherein the phenyl or the heteroaryl group is substituted at the α-position, and wherein R² may optionally be further substituted. Typically, R² is an aryl or a heteroaryl group, wherein the aryl or the heteroaryl group is substituted at the α and α′ positions, and wherein R² may optionally be further substituted. Typically, R² is a phenyl or a 5- or 6-membered heteroaryl group, wherein the phenyl or the heteroaryl group is substituted at the α and α′ positions, and wherein R² may optionally be further substituted. For example, R² may be a phenyl group substituted at the 2- and 6-positions or a phenyl group substituted at the 2-, 4- and 6-positions.

In one embodiment, the parent phenyl or 5- or 6-membered heteroaryl group of R² may be selected from phenyl, pyridinyl, pyridazinyl, pyrimidinyl, pyrazinyl, pyrrolyl, furanyl, thiophenyl, pyrazolyl, imidazolyl, oxazolyl, isoxazolyl, thiazolyl, isothiazolyl, triazolyl or oxadiazolyl. Typically, the parent phenyl or 5- or 6-membered heteroaryl group of R² may be selected from phenyl, pyridinyl, pyridazinyl, pyrimidinyl, pyrrolyl, pyrazolyl, imidazolyl or triazolyl. Typically, the parent phenyl or 5- or 6-membered heteroaryl group of R² may be selected from phenyl, pyridinyl, pyridazinyl, pyrimidinyl or pyrazolyl. More typically, the parent phenyl or 5- or 6-membered heteroaryl group of R² may be selected from phenyl or pyrazolyl.

As used herein, the nomenclature α, β, α′, β′ refers to the position of the atoms of a cyclic group, such as —R², relative to the point of attachment of the cyclic group to the remainder of the molecule. For example, where —R² is a 1,2,3,5,6,7-hexahydro-s-indacen-4-yl moiety, the α, β, α′ and β′ positions are as follows:

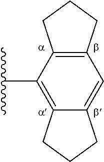 [SMILES: *c1c2c(cc3c1CCC3)CCC2]

For the avoidance of doubt, where it is stated that a cyclic group, such as an aryl or a heteroaryl group, is substituted at the α and/or α′ positions, it is to be understood that one or more hydrogen atoms at the α and/or α′ positions respectively are replaced by one or more substituents, such as any optional substituent as defined above. Unless stated otherwise, the term “substituted” does not include the replacement of one or more ring carbon atoms by one or more ring heteroatoms.

In another embodiment, R² is a cyclic group substituted at the α and α′ positions, wherein R² may optionally be further substituted. For example, R² may be a cycloalkyl, cycloalkenyl or non-aromatic heterocyclic group substituted at the α and α′ positions. In any of the above embodiments, typical substituents at the α and/or α′ positions of the parent cyclic group of R² comprise a carbon atom. For example, typical substituents at the α and/or α′ positions may be independently selected from —R⁴, —OR⁴ and —COR⁴ groups, wherein each R⁴ is independently selected from a C₁-C₆ alkyl, C₂-C₆ alkenyl, C₂-C₆ alkynyl or C₂-C₆ cyclic group and wherein each R⁴ is optionally further substituted with one or more halo groups. More typically, the substituents at the α and/or α′ positions are independently selected from alkyl and cycloalkyl groups, such as C₃-C₆ branched alkyl and C₃-C₆ cycloalkyl groups, e.g. isopropyl, cyclopropyl, cyclohexyl or t-butyl groups, wherein the alkyl and cycloalkyl groups are optionally further substituted with one or more fluoro and/or chloro groups.

In one aspect of any of the above embodiments, at least one substituent at the α and/or α′ positions comprises a carbon atom. Typically, each substituent at the α and/or α′ positions comprises a carbon atom. More typically, R² is substituted at the α and α′ positions and both substituents at the α and α′ positions comprise a carbon atom.

In a further aspect of any of the above embodiments, at least one substituent at the α and/or α′ positions comprises a sp² or sp³ hydridised carbon atom. Typically, each substituent at the α and/or α′ positions comprises a sp² or sp³ hydridised carbon atom. More typically, R² is substituted at the α and α′ positions and both substituents at the α and α′ positions comprise a sp² or sp³ hydridised carbon atom.

Typically, at least one substituent at the α and/or α′ positions comprises a sp³ hydridised carbon atom.

Other typical substituents at the α and/or α′ positions of the parent cyclic group of R² may include cycloalkyl, cycloalkenyl, non-aromatic heterocyclic, aryl or heteroaryl rings which are fused to the parent cyclic group across the α,β and/or α′,β′ positions respectively. Such fused cyclic groups are described in greater detail below.

In one embodiment, R² is a fused aryl or a fused heteroaryl group, wherein the aryl or heteroaryl group is fused to one or more cycloalkyl, cycloalkenyl, non-aromatic heterocyclic, aryl or heteroaryl rings, wherein R² may optionally be further substituted. Typically, a cycloalkyl, cycloalkenyl, non-aromatic heterocyclic, aryl or heteroaryl ring is fused to the aryl or heteroaryl group across the α,β positions. Typically, the aryl or heteroaryl group is also substituted at the α′ position, for example with a substituent selected from —R⁴, —OR⁴ and —COR⁴, wherein each R⁴ is independently selected from a C₁-C₆ alkyl, C₂-C₆ alkenyl, C₂-C₆ alkynyl or C₂-C₆ cyclic group and wherein each R⁴ is optionally further substituted with one or more halo groups. Typically in such an embodiment, R² is bicyclic or tricyclic.

More typically, R² is a fused phenyl or a fused 5- or 6-membered heteroaryl group, wherein the phenyl or the 5- or 6-membered heteroaryl group is fused to one or more cycloalkyl, cycloalkenyl, non-aromatic heterocyclic, aryl or heteroaryl rings, wherein R² may optionally be further substituted. Typically, a cycloalkyl, cycloalkenyl, non-aromatic heterocyclic, aryl or heteroaryl ring is fused to the phenyl or the 5- or 6-membered heteroaryl group across the α,β positions so as to form a 4- to 6-membered fused ring structure. Typically, the phenyl or the 5- or 6-membered heteroaryl group is also substituted at the α′ position, for example with a substituent selected from —R⁴, —OR⁴ and —COR⁴, wherein each R⁴ is independently selected from a C₁-C₆ alkyl, C₂-C₆ alkenyl, C₂-C₆ alkynyl or C₂-C₆ cyclic group and wherein each R⁴ is optionally further substituted with one or more halo groups. Typically in such an embodiment, R² is bicyclic or tricyclic.

In another embodiment, R² is a fused aryl or a fused heteroaryl group, wherein the aryl or heteroaryl group is fused to two or more independently selected cycloalkyl, cycloalkenyl, non-aromatic heterocyclic, aryl or heteroaryl rings, wherein R² may optionally be further substituted. Typically, the two or more cycloalkyl, cycloalkenyl, non-aromatic heterocyclic, aryl or heteroaryl rings are each ortho-fused to the aryl or heteroaryl group, i.e. each fused cycloalkyl, cycloalkenyl, non-aromatic heterocyclic, aryl or heteroaryl ring has only two atoms and one bond in common with the aryl or heteroaryl group. Typically in such an embodiment, R² is tricyclic.

In yet another embodiment, R² is a fused aryl or a fused heteroaryl group, wherein a first cycloalkyl, cycloalkenyl, non-aromatic heterocyclic, aryl or heteroaryl ring is fused to the aryl or heteroaryl group across the α,β positions and a second cycloalkyl, cycloalkenyl, non-aromatic heterocyclic, aryl or heteroaryl ring is fused to the aryl or heteroaryl group across the α′,β′ positions, wherein R² may optionally be further substituted. Typically in such an embodiment, R² is tricyclic.

More typically, R² is a fused phenyl or a fused 5- or 6-membered heteroaryl group, wherein a first cycloalkyl, cycloalkenyl, non-aromatic heterocyclic, aryl or heteroaryl ring is fused to the phenyl or the 5- or 6-membered heteroaryl group across the α,β positions so as to form a first 4- to 6-membered fused ring structure, and a second cycloalkyl, cycloalkenyl, non-aromatic heterocyclic, aryl or heteroaryl ring is fused to the phenyl or the 5- or 6-membered heteroaryl group across the α′,β′ positions so as to form a second 4- to 6-membered fused ring structure, wherein R² may optionally be further substituted. Typically in such an embodiment, R² is tricyclic.

In one embodiment, —R² has a formula selected from:

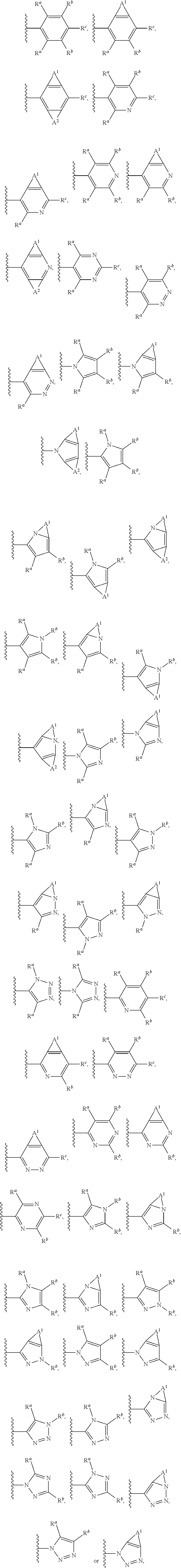

wherein:
A¹ and A² are each independently selected from an optionally substituted alkylene or alkenylene group, wherein one or more carbon atoms in the backbone of the alkylene or alkenylene group may optionally be replaced by one or more heteroatoms N, O or S;
each R^{a} is independently selected from hydrogen, halo, —R^{aa}, —OR^{aa} or —COR^{aa}, provided that at least one R^{a} is —R^{aa}, —OR^{aa} or —COR^{aa};
each R^{b} is independently selected from hydrogen, halo, —NO₂, —CN, —R^{aa}, —OR^{aa} or —COR^{aa};
provided that any R^{a} or R^{b} that is directly attached to a ring nitrogen atom is not halo, —NO₂, —CN, or —OR^{aa};
each R^{c} is independently selected from hydrogen, halo, —OH, —NO₂, —CN, —R^{cc}, —OR^{cc}, —COR^{cc}, —COOR^{cc}, —CONH₂, —CONHR^{cc}, —CON(R^{cc})₂, —C(═NH)R^{cc}, —C(═NH)NH₂, —C(═NH)NHR^{cc}, —C(═NH)N(R^{cc})₂, —C(═NR^{cc})R^{cc}, —C(═NR^{cc})NHR^{cc}, —C(═NR^{cc})N(R^{cc})₂, —C(═NOH)R^{cc} or —C(═NOR^{cc})R^{cc};
each R^{aa} is independently selected from a C₁-C₆ alkyl, C₂-C₆ alkenyl, C₂-C₆ alkynyl or a 3- to 7-membered cyclic group, wherein each R^{aa} is optionally substituted; and
each R^{cc} is independently selected from a C₁-C₆ alkyl, C₂-C₆ alkenyl, C₂-C₆ alkynyl or a 3- to 7-membered cyclic group, or any two R^{cc} attached to the same nitrogen atom may, together with the nitrogen atom to which they are attached, form a 3- to 7-membered heterocyclic group, wherein each R^{cc} is optionally substituted.

Typically in the above embodiment, —R² has a formula selected from:

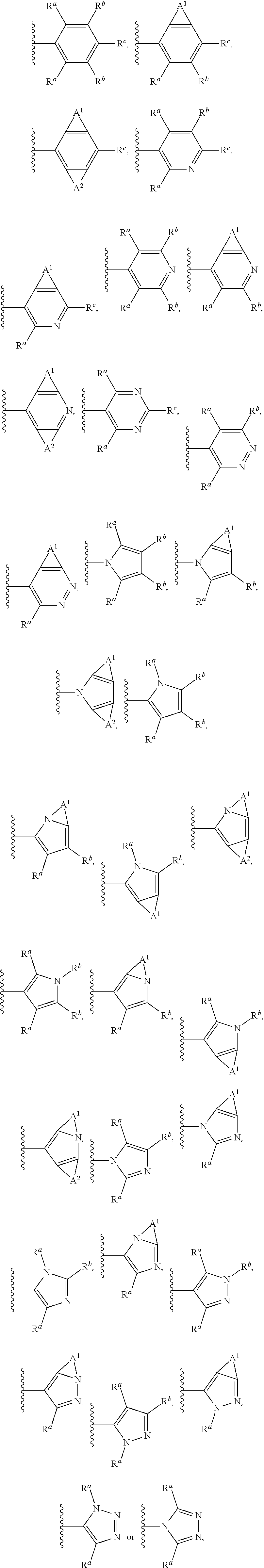

wherein each R^{a} is independently selected from —R^{aa}, —OR^{aa} or —COR^{aa}, and R^{aa}, R^{b}, R^{c}, A¹ and A² are as defined above.

More typically in the above embodiment, —R² has a formula selected from:

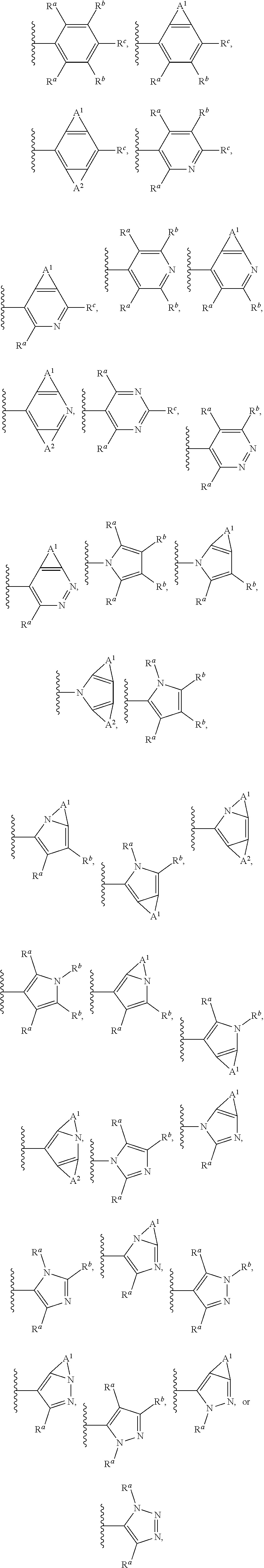

wherein each R^{a} is independently selected from —R^{aa}, —OR^{aa} or —COR^{aa}, and R^{aa}, R^{b}, R^{c}, A¹ and A² are as defined above.

Typically, R² is not connected to G via an oxygen-nitrogen or a nitrogen-nitrogen bond. For example, where G is —O— or —NR^{gg}—, —R² may have a formula selected from:

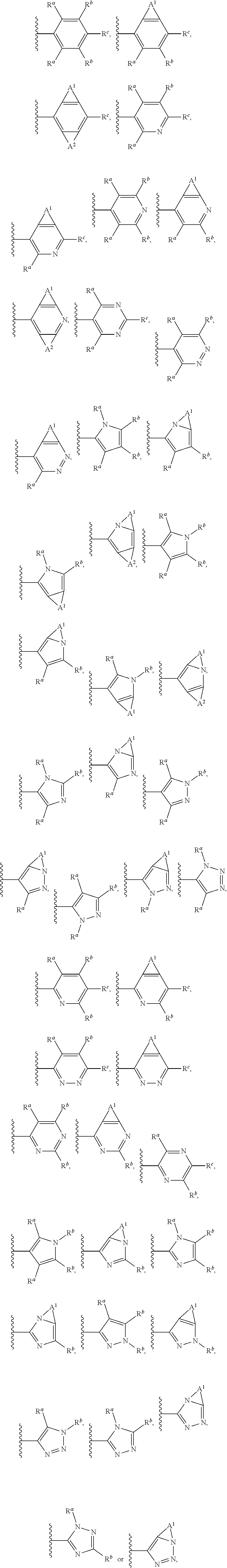

wherein R^{a}, R^{b}, R^{c}, A¹ and A² are as defined above.

More typically, where G is —O— or —NR^{gg}—, —R² has a formula selected from:

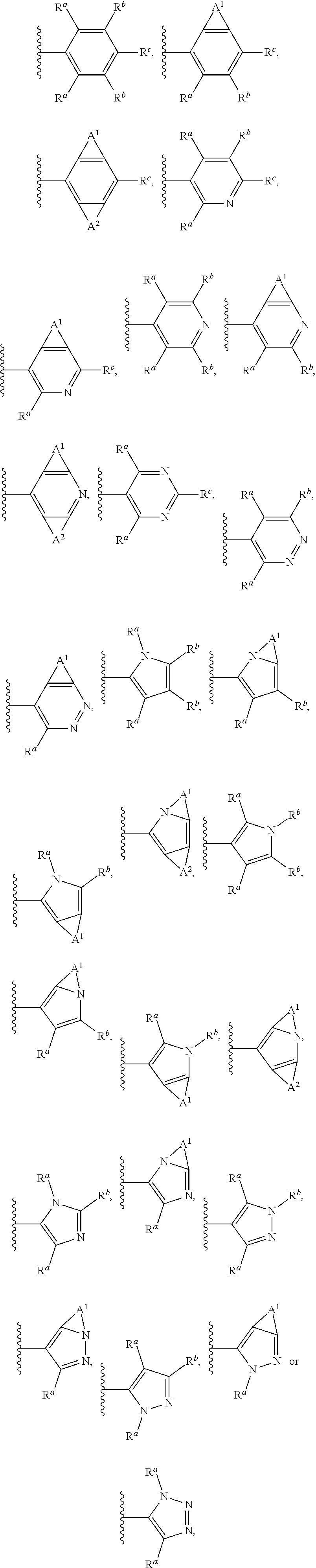

wherein each R^{a} is independently selected from —R^{aa}, —OR^{aa} or —COR^{aa}, and R^{aa}, R^{b}, R^{c}, A¹ and A² are as defined above.

More typically in any embodiment, —R² has a formula selected from:

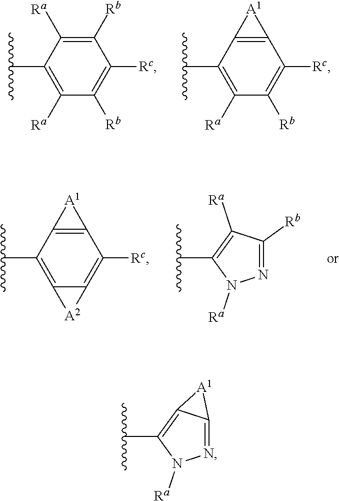

wherein R^{α}, R^{b}, R^{c}, A¹ and A² are as defined above.

More typically still, —R² has a formula selected from:

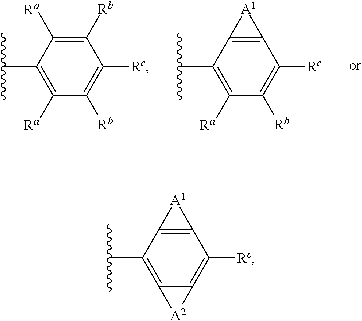

wherein each R^{a} is independently selected from —R^{aa}, —OR^{aa} or —COR^{aa}, and R^{aa}, R^{b}, R^{c}, A¹ and A² are as defined above.

Typically in any of the above embodiments, any ring containing A¹ or A² is a 5- or 6-membered ring. Typically, A¹ and A² are each independently selected from an optionally substituted straight-chained alkylene group or an optionally substituted straight-chained alkenylene group, wherein one or two carbon atoms in the backbone of the alkylene or alkenylene group may optionally be replaced by one or two heteroatoms independently selected from nitrogen and oxygen. More typically, A¹ and A² are each independently selected from an optionally substituted straight-chained alkylene group, wherein one carbon atom in the backbone of the alkylene group may optionally be replaced by an oxygen atom. Typically, no heteroatom in A¹ or A² is directly attached to another ring heteroatom. Typically, A¹ and A² are unsubstituted or substituted with one or more substituents independently selected from halo, —OH, —CN, —NO₂, C₁-C₄ alkyl, C₁-C₄ haloalkyl, —O(C₁-C₄ alkyl) or —O(C₁-C₄ haloalkyl). More typically, A¹ and A² are unsubstituted or substituted with one or more fluoro and/or chloro groups. Where R² contains both A¹ and A² groups, A¹ and A² may be the same or different. Typically, A¹ and A² are the same.

Where R^{aa} is a substituted C₁-C₆ alkyl, C₂-C₆ alkenyl or C₂-C₆ alkynyl group, typically the C₁-C₆ alkyl, C₂-C₆ alkenyl or C₂-C₆ alkynyl group is substituted with one or more (e.g. one or two) substituents independently selected from halo, —OH, —CN, —NO₂, —O(C₁-C₄ alkyl) or —O(C₁-C₄ haloalkyl).

Where R^{aa} is a substituted 3- to 7-membered cyclic group, typically the 3- to 7-membered cyclic group is substituted with one or more (e.g. one or two) substituents independently selected from halo, —OH, —NH₂, —CN, —NO₂, —B¹, —CH₂B¹, —OB¹, —OCH₂B¹, —NHB¹, —N(B¹)₂, —CONH₂, —CONHB¹, —CON(B¹)₂, —NHCOB¹, —NBCOB¹, or —B¹¹—;
wherein each B¹ is independently selected from a C₁-C₆ alkyl, C₂-C₆ alkenyl, C₂-C₆ alkynyl, C₃-C₁₀ cycloalkyl, C₅-C₁₀ cycloalkenyl, C₆-C₁₀ aryl, or a 4- to 10-membered heterocyclic group containing one or two ring heteroatoms N and/or O, or two B¹ together with the nitrogen atom to which they are attached may form a 4- to 10-membered heterocyclic group containing one or two ring heteroatoms N and/or O, wherein any B¹ may optionally be halo-substituted and/or substituted with one or two substituents independently selected from —OH, —NH₂, —B¹², —OB¹², —NHB¹² or —N(B¹²)₂;
wherein each B¹¹ is independently selected from a C₁-C₈ alkylene or C₂-C₈ alkenylene group, wherein one or two carbon atoms in the backbone of the alkylene or alkenylene group may optionally be replaced by one or two heteroatoms N and/or O, and wherein the alkylene or alkenylene group may optionally be halo-substituted and/or substituted with one or two substituents independently selected from —OH, —NH₂, —B¹², —OB¹², —NHB¹² or —N(B¹²)₂; and
wherein each B¹² is independently selected from a C₁-C₃ alkyl or C₁-C₃ haloalkyl group. Typically, any divalent group —B¹¹— forms a 4- to 6-membered fused ring.

Typically in such an embodiment, each B¹ is independently selected from a C₁-C₄ alkyl, C₂-C₄ alkenyl, C₂-C₄ alkynyl, C₃-C₆ cycloalkyl or phenyl group, or a 4- to 6-membered heterocyclic group containing one or two ring heteroatoms N and/or O, or two B¹ together with the nitrogen atom to which they are attached may form a 4- to 6-membered heterocyclic group containing one or two ring heteroatoms N and/or O, wherein any B¹ may optionally be halo-substituted and/or substituted with one or two substituents independently selected from —OH, —NH₂, —B¹², —OB¹², —NHB¹² or —N(B¹²)₂, and wherein B¹² is as defined above.

In one embodiment, each R^{aa} is independently selected from a C₁-C₄ alkyl or a 3- to 6-membered cyclic group, wherein each C₁-C₄ alkyl group is optionally substituted with one or more halo substituents and/or one or two substituents independently selected from —OH, —CN, —O(C₁-C₄ alkyl) or —O(C₁-C₄ haloalkyl), and wherein each 3- to 6-membered cyclic group is optionally substituted with one or more halo substituents and/or one or two substituents independently selected from halo, —OH, —CN, —B¹, —CH₂B¹, —OB¹ or —OCH₂B¹;
wherein each B¹ is independently selected from a C₁-C₄ alkyl, C₃-C₆ cycloalkyl or phenyl group, or a 4- to 6-membered heterocyclic group containing one or two ring heteroatoms N and/or O, and wherein any B¹ may optionally be halo-substituted and/or substituted with one or two substituents independently selected from —OH, —B¹² or —OB¹²; and
each B¹² is independently selected from a C₁-C₃ alkyl or C₁-C₃ haloalkyl group.

In one embodiment, each R^{a} is independently selected from hydrogen, halo or —R^{aa}, provided that at least one R^{a} is —R^{aa}. Typically, each R^{a} is —R^{aa}. In one embodiment, each R^{a} is independently selected from a C₁-C₆ alkyl (in particular C₃-C₆ branched alkyl) or C₃-C₆ cycloalkyl group, wherein each R^{a} is optionally further substituted with one or more halo groups. More typically, each R^{a} is independently selected from a C₁-C₄ alkyl, C₁-C₄ haloalkyl, C₃-C₄ cycloalkyl or C₃-C₄ halocycloalkyl group. Where a group R^{a} is present at both the α- and α′-positions, each R^{a} may be the same or different. Typically, each R^{a} is the same.

In one embodiment, each R^{b} is independently selected from hydrogen, halo, methyl or fluoromethyl. Typically, each R^{b} is independently selected from hydrogen or halo. More typically, each R^{b} is hydrogen.

In one embodiment, R^{c} is selected from hydrogen, halo, —OH, —NO₂, —CN, —R^{cc}, —R^{cx}, —OR^{cc}, —COR^{cc}, —COOR^{cc}, —CONH₂, —CONHR^{cc}, —CON(R^{cc})₂, —C(═NH)R^{cc}, —C(═NH)NH₂, —C(═NH)NHR^{cc}, —C(═NH)N(R^{cc})₂, —C(═NR^{cc})R^{cc}, —C(═NR^{cc})NHR^{cc}, —C(═NR^{cc})N(R^{cc})₂, —C(═NOH)R^{cc} or —C(═NOR^{cc})R^{cc} wherein each —R^{cc} is independently selected from C₁-C₄ alkyl, C₁-C₄ haloalkyl, C₃-C₄ cycloalkyl or C₃-C₄ halocycloalkyl, or any two R^{cc} attached to the same nitrogen atom may, together with the nitrogen atom to which they are attached, form a 3- to 6-membered saturated heterocyclic group, wherein the 3- to 6-membered saturated heterocyclic group is optionally halo substituted, and wherein —R^{cx}— is selected from a 3- to 7-membered cyclic group, wherein the 3- to 7-membered cyclic group is optionally halo substituted. Typically, R^{c} is selected from hydrogen, halo, —CN, —R^{cc}, —R^{cx}—, —OR^{cc}, —COR^{cc}, —C(═NOH)R^{cc} or —C(═NOR^{cc})R^{cc}, wherein each —R^{cc} is independently selected from C₁-C₃ alkyl, C₁-C₃ fluoroalkyl, cyclopropyl or fluorocyclopropyl, and wherein —R^{cx} is selected from a phenyl, halophenyl or a 5- or 6-membered heteroaryl group, wherein the 5- or 6-membered heteroaryl group is optionally halo substituted. More typically, R^{c} is independently selected from hydrogen, —CN or halo.

In one embodiment, —R² has a formula selected from:

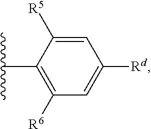

wherein R⁵ and R⁶ are independently selected from C₁-C₄ alkyl, C₁-C₄ haloalkyl, C₃-C₄ cycloalkyl and C₃-C₄ halocycloalkyl, and R^{d} is hydrogen, halo, —OH, —NO₂, —CN, —R^{dd}, —R^{dx}, —OR^{dd}, —COR^{dd}, —COOR^{dd}, —CONH₂, —CONHR^{dd}, —CON(R^{dd})₂, —C(═NH)R^{dd}, —C(═NH)NH₂, —C(═NH)NHR^{dd}, —C(═NH)N(R^{dd})₂, —C(═NR^{dd})R^{dd}, —C(═NR^{dd})NHR^{dd}, —C(═NR^{dd})N(R^{dd})₂, —C(═NOH)R^{dd} or —C(═NOR^{dd})R^{dd}, wherein each —R^{dd} is independently selected from C₁-C₄ alkyl, C₁-C₄ haloalkyl, C₃-C₄ cycloalkyl and C₃-C₄ halocycloalkyl, or any two R^{dd} attached to the same nitrogen atom may, together with the nitrogen atom to which they are attached, form a 3- to 6-membered saturated heterocyclic group, wherein the 3- to 6-membered saturated heterocyclic group is optionally halo substituted, and wherein —R^{d} is selected from a 3- to 7-membered cyclic group, wherein the 3- to 7-membered cyclic group is optionally halo substituted. Typically, R⁵ and R⁶ are independently selected from C₁-C₄ alkyl, and R^{d} is hydrogen, halo, —CN, —R^{dd}, —R^{dx}, —OR^{dd}, —COR^{dd}, —C(═NOH)R^{dd} or —C(═NOR^{dd})R^{dd}, wherein each —R^{dd} is independently selected from C₁-C₃ alkyl, C₁-C₃ fluoroalkyl, cyclopropyl or fluorocyclopropyl, and wherein —R^{dx} is selected from a phenyl, halophenyl or a 5- or 6-membered heteroaryl group, wherein the 5- or 6-membered heteroaryl group is optionally halo substituted. More typically, R⁵ and R⁶ are independently selected from C₁-C₄ alkyl, and R^{d} is hydrogen or a halo group.

Typically, —R² has a formula selected from:

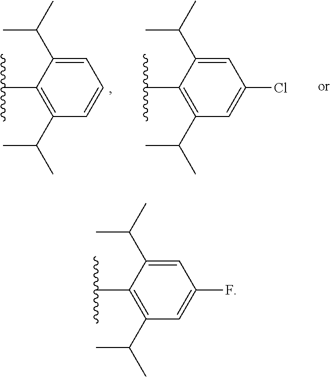

In one embodiment, —R² has a formula selected from:

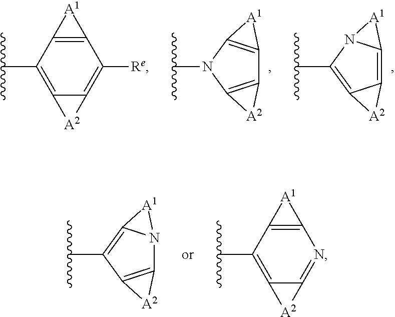

wherein A¹ and A² are each independently selected from an optionally substituted alkylene or alkenylene group, wherein one or more carbon atoms in the backbone of the alkylene or alkenylene group may optionally be replaced by one or more heteroatoms N, O or S, and wherein R^{e} is hydrogen or any optional substituent. R^{e} and any optional substituent attached to A¹ or A² may together with the atoms to which they are attached form a further fused cycloalkyl, cycloalkenyl, non-aromatic heterocyclic, aryl or heteroaryl ring which may itself be optionally substituted. Similarly, any optional substituent attached to A¹ and any optional substituent attached to A² may also together with the atoms to which they are attached form a further fused cycloalkyl, cycloalkenyl, non-aromatic heterocyclic, aryl or heteroaryl ring which may itself be optionally substituted.

In one embodiment, R^{e} is hydrogen, halo, —OH, —NO₂, —CN, —R^{ee}, —R^{ex}, —OR^{ee}, —COR^{ee}, —COOR^{ee}, —CONH₂, —CONHR^{ee}, —CON(R^{ee})₂, —C(═NH)R^{ee}, —C(═NH)NH₂, —C(═NH)NHR^{ee}, —C(═NH)N(R^{ee})₂, —C(═NR^{ee})R^{ee}, —C(═NR^{ee})NHR^{ee}, —C(═NR^{ee})N(R^{ee})₂, —C(═NOH)R^{ee} or —C(═NOR^{ee})R^{ee}, wherein each —R^{ee} is independently selected from C₁-C₄ alkyl, C₁-C₄ haloalkyl, C₃-C₄ cycloalkyl and C₃-C₄ halocycloalkyl, or any two R^{ee} attached to the same nitrogen atom may, together with the nitrogen atom to which they are attached, form a 3- to 6-membered saturated heterocyclic group, wherein the 3- to 6-membered saturated heterocyclic group is optionally halo substituted, and wherein —R^{ex} is selected from a 3- to 7-membered cyclic group, wherein the 3- to 7-membered cyclic group is optionally halo substituted. Typically, R^{e} is hydrogen, halo, —CN, —R^{ee}, —R^{ex}, —OR^{ee}, —COR^{ee}, —C(═NOH)R^{ee} or —C(═NOR^{ee})R^{ee}, wherein each —R^{ee} is independently selected from C₁-C₃ alkyl, C₁-C₃ fluoroalkyl, cyclopropyl or fluorocyclopropyl, and wherein —R^{ex} is selected from a phenyl, halophenyl or a 5- or 6-membered heteroaryl group, wherein the 5- or 6-membered heteroaryl group is optionally halo substituted.

In another embodiment, R^{e} is hydrogen or a halo, hydroxyl, —CN, —NO₂, —R^{ee} or —OR^{ee} group, wherein R^{ee} is a C₁-C₄ alkyl group which may optionally be halo-substituted. More typically, R^{e} is hydrogen or halo.

Typically, any ring containing A¹ or A² is a 5- or 6-membered ring. Typically, A¹ and A² are each independently selected from an optionally substituted straight-chained alkylene group or an optionally substituted straight-chained alkenylene group, wherein one or two carbon atoms in the backbone of the alkylene or alkenylene group may optionally be replaced by one or two heteroatoms independently selected from nitrogen and oxygen. More typically, A¹ and A² are each independently selected from an optionally substituted straight-chained alkylene group, wherein one carbon atom in the backbone of the alkylene group may optionally be replaced by an oxygen atom. Typically, no heteroatom in A¹ or A² is directly attached to another ring heteroatom. Typically, A¹ and A² are unsubstituted or substituted with one or more halo, hydroxyl, —CN, —NO₂, —B³ or —OB³ groups, wherein B³ is a C₁-C₄ alkyl group which may optionally be halo-substituted. More typically, A¹ and A² are unsubstituted or substituted with one or more fluoro and/or chloro groups. Where R² contains both A and A² groups, A¹ and A² may be the same or different. Typically, A¹ and A² are the same.

In a further embodiment, —R² has a formula selected from:

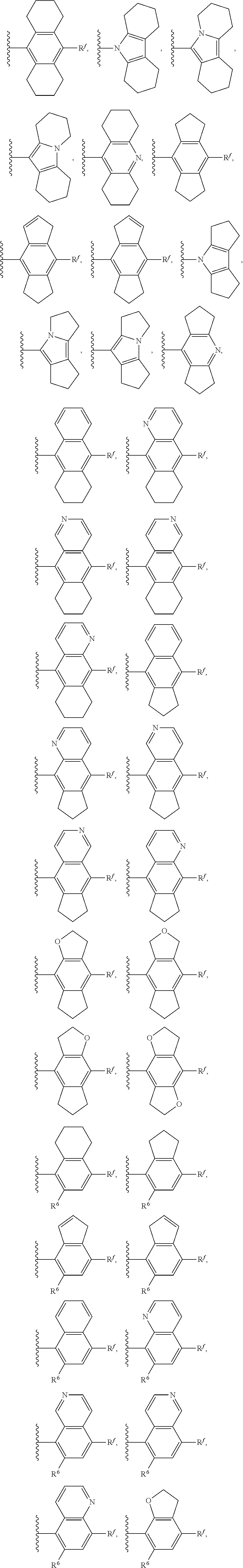

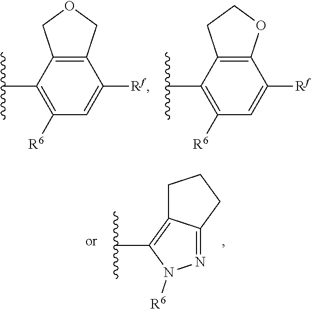

wherein R⁶ is C₁-C₄ alkyl, C₁-C₄ haloalkyl, C₃-C₄ cycloalkyl or C₃-C₄ halocycloalkyl, and R^{f} is hydrogen, halo, —OH, —NO₂, —CN, —R^{ff}, —R^{fx}, —OR^{ff}, —COR^{ff}, —COOR^{ff}, —CONH₂, —CONHR^{ff}, —CON(R^{ff})₂, —C(═NH)R^{ff}, —C(═NH)NH₂, —C(═NH)NHR^{ff}, —C(═NH)N(R^{ff})₂, —C(═NR^{ff})R^{ff}, —C(═NR^{ff})NHR^{ff}, —C(═NR^{ff})N(R^{ff})₂, —C(═NOH)R^{ff} or —C(═NOR^{ff})R^{ff}, wherein each —R^{ff} is independently selected from C₁-C₄ alkyl, C₁-C₄ haloalkyl, C₃-C₄ cycloalkyl and C₃-C₄ halocycloalkyl, or any two R^{ff} attached to the same nitrogen atom may, together with the nitrogen atom to which they are attached, form a 3- to 6-membered saturated heterocyclic group, wherein the 3- to 6-membered saturated heterocyclic group is optionally halo substituted, and wherein —R^{fx} is selected from a 3- to 7-membered cyclic group, wherein the 3- to 7-membered cyclic group is optionally halo substituted. Typically, R⁶ is C₁-C₄ alkyl, and R^{f} is hydrogen, halo, —CN, —R^{ff}, —R^{fx}, —OR^{ff}, —COR^{ff}, —C(═NOH)R^{ff} or —C(═NOR^{ff})R^{ff}, wherein each —R^{ff} is independently selected from C₁-C₃ alkyl, C₁-C₃ fluoroalkyl, cyclopropyl or fluorocyclopropyl, and wherein —R^{fx} is selected from a phenyl, halophenyl or a 5- or 6-membered heteroaryl group, wherein the 5- or 6-membered heteroaryl group is optionally halo substituted. More typically, R⁶ is C₁-C₄ alkyl, and R^{f} is hydrogen or halo.

Typically, —R² has the formula:

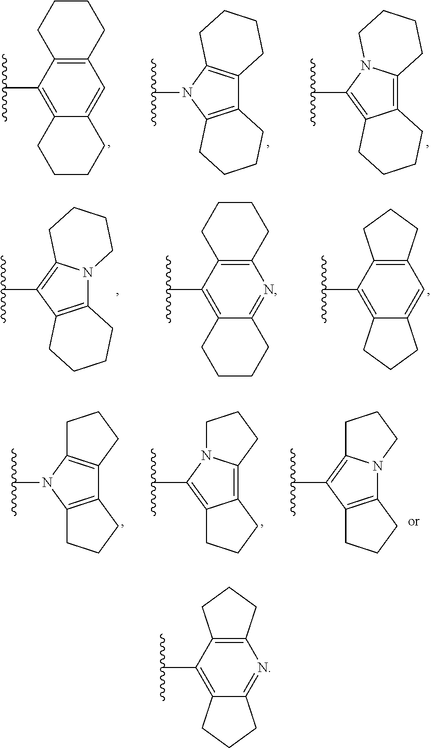

Typically, —R² has the formula:

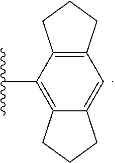 [SMILES: *c1c2c(cc3c1CCC3)CCC2]

Yet other typical substituents at the α-position of the parent cyclic group of R² may include monovalent heterocyclic groups and monovalent aromatic groups, wherein a ring atom of the heterocyclic or aromatic group is directly attached via a single bond to the α-ring atom of the parent cyclic group, wherein the heterocyclic or aromatic group may optionally be substituted, and wherein the parent cyclic group may optionally be further substituted. Such R² groups are described in greater detail below.

In one embodiment, the α-substituted parent cyclic group of R² is a 5- or 6-membered cyclic group, wherein the cyclic group may optionally be further substituted. In one embodiment, the α-substituted parent cyclic group of R² is an aryl or a heteroaryl group, all of which may optionally be further substituted. In one embodiment, the α-substituted parent cyclic group of R² is a phenyl or a 5- or 6-membered heteroaryl group, all of which may optionally be further substituted. In one embodiment, the α-substituted parent cyclic group of R² is a phenyl, pyridinyl, pyridazinyl, pyrimidinyl, pyrazinyl, pyrrolyl, furanyl, thiophenyl, pyrazolyl, imidazolyl, oxazolyl, isoxazolyl, thiazolyl, isothiazolyl, triazolyl or oxadiazolyl group, all of which may optionally be further substituted. In one embodiment, the α-substituted parent cyclic group of R² is a phenyl or pyrazolyl group, both of which may optionally be further substituted. In a further embodiment, the α-substituted parent cyclic group of R² is a phenyl group, which may optionally be further substituted.

In one embodiment, the α-substituted parent cyclic group of R² is substituted at the α and α′ positions, and may optionally be further substituted. For example, the α-substituted parent cyclic group of R² may be a phenyl group substituted at the 2- and 6-positions or a phenyl group substituted at the 2-, 4- and 6-positions.

In one embodiment, R² is a parent cyclic group substituted at the α-position with a monovalent heterocyclic group or a monovalent aromatic group, wherein the heterocyclic or aromatic group may optionally be substituted, and wherein the parent cyclic group may optionally be further substituted. In one embodiment, the monovalent heterocyclic or aromatic group at the α-position is a phenyl or a 5- or 6-membered heterocyclic group, all of which may optionally be substituted. In one embodiment, the monovalent heterocyclic or aromatic group at the α-position is a phenyl, pyridinyl, pyridazinyl, pyrimidinyl, pyrazinyl, pyrrolyl, furanyl, thiophenyl, pyrazolyl, imidazolyl, oxazolyl, isoxazolyl, thiazolyl, isothiazolyl, triazolyl, oxadiazolyl, azetinyl, azetidinyl, oxetanyl, thietanyl, pyrrolidinyl, tetrahydrofuranyl, tetrahydrothiophenyl, pyrazolidinyl, imidazolidinyl, 1,3-dioxolanyl, 1,2-oxathiolanyl, 1,3-oxathiolanyl, piperidinyl, tetrahydropyranyl, piperazinyl, 1,4-dioxanyl, thianyl, morpholinyl, thiomorpholinyl or 1-methyl-2-oxo-1,2-dihydropyridinyl group, all of which may optionally be substituted. In one embodiment, the monovalent heterocyclic or aromatic group at the α-position is a phenyl, pyridinyl, pyridazinyl, pyrimidinyl, pyrazinyl, pyrrolyl, furanyl, thiophenyl, pyrazolyl, imidazolyl, oxazolyl, isoxazolyl, thiazolyl, isothiazolyl, triazolyl, oxadiazolyl, azetinyl, azetidinyl, oxetanyl, thietanyl, pyrrolidinyl, tetrahydrofuranyl, tetrahydrothiophenyl, pyrazolidinyl, imidazolidinyl, 1,3-dioxolanyl, 1,2-oxathiolanyl, 1,3-oxathiolanyl, piperidinyl, tetrahydropyranyl, thianyl, piperazinyl, 1,4-dioxanyl, morpholinyl or thiomorpholinyl group, all of which may optionally be substituted. In one embodiment, the monovalent heterocyclic or aromatic group at the α-position is a phenyl, pyridinyl, pyridazinyl, pyrimidinyl, pyrazinyl, pyrrolyl, furanyl, thiophenyl, pyrazolyl, imidazolyl, oxazolyl, isoxazolyl, thiazolyl, isothiazolyl, piperidinyl or tetrahydropyranyl group, all of which may optionally be substituted. In one embodiment, the monovalent heterocyclic or aromatic group at the α-position is a phenyl, pyridinyl, pyridazinyl, pyrimidinyl, pyrazolyl, imidazolyl, isoxazolyl, thiazolyl, tetrahydropyranyl or 1-methyl-2-oxo-1,2-dihydropyridinyl group, all of which may optionally be substituted. In one embodiment, the monovalent heterocyclic or aromatic group at the α-position is a phenyl, pyridinyl, pyrimidinyl, pyrazolyl, imidazolyl, isoxazolyl, thiazolyl or tetrahydropyranyl group, all of which may optionally be substituted. In one embodiment, the monovalent heterocyclic or aromatic group at the α-position is a phenyl, pyridinyl, pyrimidinyl or pyrazolyl group, all of which may optionally be substituted. In one embodiment, the monovalent heterocyclic or aromatic group at the α-position is an unsubstituted phenyl, pyridinyl, pyrimidinyl or pyrazolyl group. In one embodiment, the monovalent heterocyclic group at the α-position is a pyridin-2-yl, pyridin-3-yl or pyridin-4-yl group, all of which may optionally be substituted. In one embodiment, the monovalent heterocyclic group at the α-position is an unsubstituted pyridin-3-yl group or an optionally substituted pyridin-4-yl group.

For any of these monovalent heterocyclic or aromatic groups at the α-position mentioned in the immediately preceding paragraph, the monovalent heterocyclic or aromatic group may optionally be substituted with one or two substituents independently selected from halo, —OH, —NH₂, —CN, —NO₂, —B⁴, —CH₂B⁴, —OB⁴, —OCH₂B⁴, —NHB⁴, —N(B⁴)₂, —CONH₂, —CONHB⁴, —CON(B⁴)₂, —NHCOB⁴, —NB⁴COB⁴, or —B⁴⁴—;
wherein each B⁴ is independently selected from a C₁-C₄ alkyl, C₂-C₄ alkenyl, C₂-C₄ alkynyl, C₃-C₆ cycloalkyl or phenyl group, or a 4- to 6-membered heterocyclic group containing one or two ring heteroatoms N and/or O, or two B⁴ together with the nitrogen atom to which they are attached may form a 4- to 6-membered heterocyclic group containing one or two ring heteroatoms N and/or O wherein any B⁴ may optionally be halo-substituted and/or substituted with one or two substituents independently selected from —OH, —NH₂, —B⁴⁵, —OB⁴⁵, —NHB⁴⁵ or —N(B⁴⁵)₂;
wherein each B⁴⁴ is independently selected from a C₁-C₈ alkylene or C₂-C₈ alkenylene group, wherein one or two carbon atoms in the backbone of the alkylene or alkenylene group may optionally be replaced by one or two heteroatoms N and/or O, and wherein the alkylene or alkenylene group may optionally be halo-substituted and/or substituted with one or two substituents independently selected from —OH, —NH₂, —B⁴⁵, —OB⁴⁵, —NHB⁴⁵ or —N(B⁴⁵)₂; and
wherein each B⁴⁵ is independently selected from a C₁-C₃ alkyl or C₁-C₃ haloalkyl group.

Typically, any divalent group —B⁴⁴— forms a 4- to 6-membered fused ring.

In one embodiment, the monovalent heterocyclic or aromatic group at the α-position is a phenyl, pyridinyl, pyrimidinyl or pyrazolyl group, all of which may optionally be substituted with one or two substituents independently selected from halo, —OH, —NH₂, —CN, —NO₂, —B⁴, —OB⁴, —NHB⁴ or —N(B⁴)₂, wherein each B⁴ is independently selected from a C₁-C₄ alkyl, C₂-C₄ alkenyl or C₂-C₄ alkynyl group all of which may optionally be halo-substituted. In one embodiment, the monovalent heterocyclic group at the α-position is a pyridin-2-yl, pyridin-3-yl or pyridin-4-yl group, all of which may optionally be substituted with one or two substituents independently selected from halo, —OH, —NH₂, —CN, —NO₂, —B⁴, —OB⁴, —NHB⁴ or —N(B⁴)₂, wherein each B⁴ is independently selected from a C₁-C₄ alkyl, C₂-C₄ alkenyl or C₂-C₄ alkynyl group all of which may optionally be halo-substituted. In one embodiment, the monovalent heterocyclic group at the α-position is an unsubstituted pyridin-3-yl group or a pyridin-4-yl group optionally substituted with one or two substituents independently selected from halo, —OH, —NH₂, —CN, —NO₂, —B⁴, —OB⁴, —NHB⁴ or —N(B⁴)₂, wherein each B⁴ is independently selected from a C₁-C₄ alkyl, C₂-C₄ alkenyl or C₂-C₄ alkynyl group all of which may optionally be halo-substituted.

In one embodiment, R² is a parent cyclic group substituted at the α-position with a monovalent heterocyclic group or a monovalent aromatic group, wherein the heterocyclic or aromatic group may optionally be substituted, and wherein the parent cyclic group may optionally be further substituted. In one embodiment, such further substituents are in the α′ position of the α-substituted parent cyclic group of R². Such further substituents may be independently selected from halo, —R^{δ}, —OR^{δ} or —COR^{δ} groups, wherein each R^{δ} is independently selected from a C₁-C₆ alkyl, C₂-C₆ alkenyl, C₂-C₆ alkynyl or C₂-C₆ cyclic group and wherein each R^{δ} is optionally further substituted with one or more halo groups. Typically, such further substituents on the α-substituted parent cyclic group of R² are independently selected from halo, C₁-C₆ alkyl (in particular C₃-C₆ branched alkyl) or C₃-C₆ cycloalkyl groups, e.g. fluoro, chloro, isopropyl, cyclopropyl, cyclohexyl or t-butyl groups, wherein the alkyl and cycloalkyl groups are optionally further substituted with one or more fluoro and/or chloro groups.

In one embodiment, —R² has a formula selected from:

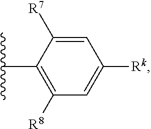

wherein R7 is C₁-C₄ alkyl, C₁-C₄ haloalkyl, C₃-C₆ cycloalkyl or C₃-C₆ halocycloalkyl, R⁸ is a 5- or 6-membered, optionally substituted heterocyclic or aromatic group, and R^{k} is hydrogen, halo, —OH, —NO₂, —CN, —R^{kk}, —R^{kx}, —OR^{kk}, —COR^{kk}, —COOR^{kk}, —CONH₂, —CONHR^{kk}, —CON(R^{kk})₂, —C(═NH)R^{kk}, —C(═NH)NH₂, —C(═NH)NHR^{kk}, —C(═NH)N(R^{kk})₂, —C(═NR^{kk})R^{kk}, —C(═NR^{kk})NHR^{kk}, —C(═NR^{kk})N(R^{kk})₂, —C(═NOH)R^{kk} or —C(═NOR^{kk})R^{kk}, wherein each —R^{kk} is independently selected from C₁-C₄ alkyl, C₁-C₄ haloalkyl, C₃-C₄ cycloalkyl and C₃-C₄ halocycloalkyl, or any two R^{kk} attached to the same nitrogen atom may, together with the nitrogen atom to which they are attached, form a 3- to 6-membered saturated heterocyclic group, wherein the 3- to 6-membered saturated heterocyclic group is optionally halo substituted, and wherein —R^{kx} is selected from a 3- to 7-membered cyclic group, wherein the 3- to 7-membered cyclic group is optionally halo substituted. In one embodiment, the optional substituents on the heterocyclic or aromatic group of R⁸ are independently selected from halo, —OH, —NH₂, —CN, —NO₂, —B⁵, —CH₂B⁵, —OB⁵, —OCH₂B⁵, —NHB⁵, —N(B⁵)₂, —CONH₂, —CONHB⁵, —CON(B⁵)₂, —NHCOB⁵, —NB⁵COB⁵, or —B⁵⁵—;
wherein each B⁵ is independently selected from a C₁-C₄ alkyl, C₂-C₄ alkenyl, C₂-C₄ alkynyl, C₃-C₆ cycloalkyl or phenyl group, or a 4- to 6-membered heterocyclic group containing one or two ring heteroatoms N and/or O, or two B⁵ together with the nitrogen atom to which they are attached may form a 4- to 6-membered heterocyclic group containing one or two ring heteroatoms N and/or O, wherein any B⁵ may optionally be halo-substituted and/or substituted with one or two substituents independently selected from —OH, —NH₂, —B⁵⁶, —OB⁵⁶, —NHB⁵⁶ or —N(B⁵⁶)
wherein each B⁵⁵ is independently selected from a C₁-C₈ alkylene or C₂-C₈ alkenylene group, wherein one or two carbon atoms in the backbone of the alkylene or alkenylene group may optionally be replaced by one or two heteroatoms N and/or O, and wherein the alkylene or alkenylene group may optionally be halo-substituted and/or substituted with one or two substituents independently selected from —OH, —NH₂, —B⁵⁶, —OB⁵⁶, —NHB⁵⁶ or —N(B⁵⁶)₂; and
wherein each B⁵⁶ is independently selected from a C₁-C₃ alkyl or C₁-C₃ haloalkyl group.

Typically, any divalent group —B⁵⁵— forms a 4- to 6-membered fused ring. Typically, R⁷ is C₁-C₄ alkyl, R⁸ is a 5- or 6-membered, optionally substituted heterocyclic or aromatic group, and R^{k} is hydrogen, halo, —CN, —R^{kk}, —R^{kx}, —OR^{kk}, —COR^{kk}, —C(═NOH)R^{kk} or —C(═NOR^{kk})R^{kk}, wherein each —R^{kk} is independently selected from C₁-C₃ alkyl, C₁-C₃ fluoroalkyl, cyclopropyl or fluorocyclopropyl, and wherein —R^{k} is selected from a phenyl, halophenyl or a 5- or 6-membered heteroaryl group, wherein the 5- or 6-membered heteroaryl group is optionally halo substituted. More typically, R⁷ is C₁-C₄ alkyl, R⁸ is a 5- or 6-membered, optionally substituted heterocyclic or aromatic group, and R^{k} is hydrogen or halo. In one embodiment, the optional substituents on the heterocyclic or aromatic group of R⁸ are independently selected from halo, —OH, —NH₂, —CN, —NO₂, —B⁵, —OB⁵, —NHB⁵ or —N(B⁵)₂, wherein each B⁵ is independently selected from a C₁-C₄ alkyl, C₂-C₄ alkenyl or C₂-C₄ alkynyl group all of which may optionally be halo-substituted.

Typically, —R² has a formula selected from:

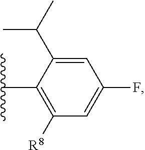

wherein R⁸ is a 5- or 6-membered, optionally substituted heterocyclic or aromatic group. In one embodiment, the optional substituents on the heterocyclic or aromatic group of R⁸ are independently selected from halo, —OH, —NH₂, —CN, —NO₂, —B⁶, —CH₂B⁶, —OB⁶, —OCH₂B⁶, —NHB⁶, —N(B⁶)₂, —CONH₂, —CONHB⁶, —CON(B⁶)₂, —NHCOB⁶, —NB⁶COB⁶, or —B⁶⁶—;
wherein each B⁶ is independently selected from a C₁-C₄ alkyl, C₂-C₄ alkenyl, C₂-C₄ alkynyl, C₃-C₆ cycloalkyl or phenyl group, or a 4- to 6-membered heterocyclic group containing one or two ring heteroatoms N and/or O, or two B⁶ together with the nitrogen atom to which they are attached may form a 4- to 6-membered heterocyclic group containing one or two ring heteroatoms N and/or O, wherein any B⁶ may optionally be halo-substituted and/or substituted with one or two substituents independently selected from —OH, —NH₂, —B⁶⁷, —OB⁶⁷, —NHB⁶⁷ or —N(B⁶⁷)₂;
wherein each B⁶⁶ is independently selected from a C₁-C₈ alkylene or C₂-C₈ alkenylene group, wherein one or two carbon atoms in the backbone of the alkylene or alkenylene group may optionally be replaced by one or two heteroatoms N and/or O, and wherein the alkylene or alkenylene group may optionally be halo-substituted and/or substituted with one or two substituents independently selected from —OH, —NH₂, —B⁶⁷, —OB⁶⁷, —NHB⁶⁷ or —N(B⁶⁷)₂; and
wherein each B⁶⁷ is independently selected from a C₁-C₃ alkyl or C₁-C₃ haloalkyl group.

Typically, any divalent group —B⁶⁶— forms a 4- to 6-membered fused ring. Typically, the optional substituents on the heterocyclic or aromatic group of R⁸ are independently selected from halo, —OH, —NH₂, —CN, —NO₂, —B⁶, —OB⁶, —NHB⁶ or —N(B⁶)₂, wherein each B⁶ is independently selected from a C₁-C₄ alkyl, C₂-C₄ alkenyl or C₂-C₄ alkynyl group all of which may optionally be halo-substituted.

In one embodiment, R² is a parent cyclic group substituted at the α-position with a monovalent heterocyclic group or a monovalent aromatic group, wherein the heterocyclic or aromatic group may optionally be substituted, and wherein the parent cyclic group may optionally be further substituted. The further substituents on the α-substituted parent cyclic group of R² also include cycloalkyl, cycloalkenyl, non-aromatic heterocyclic, aryl or heteroaryl rings which are fused to the α-substituted parent cyclic group of R². Typically, the cycloalkyl, cycloalkenyl, non-aromatic heterocyclic, aryl or heteroaryl rings are ortho-fused to the α-substituted parent cyclic group of R², i.e. each fused cycloalkyl, cycloalkenyl, non-aromatic heterocyclic, aryl or heteroaryl ring has only two atoms and one bond in common with the α-substituted parent cyclic group of R². Typically, the cycloalkyl, cycloalkenyl, non-aromatic heterocyclic, aryl or heteroaryl rings are ortho-fused to the α-substituted parent cyclic group of R² across the α′,β′ positions.

In one embodiment, —R² has a formula selected from:

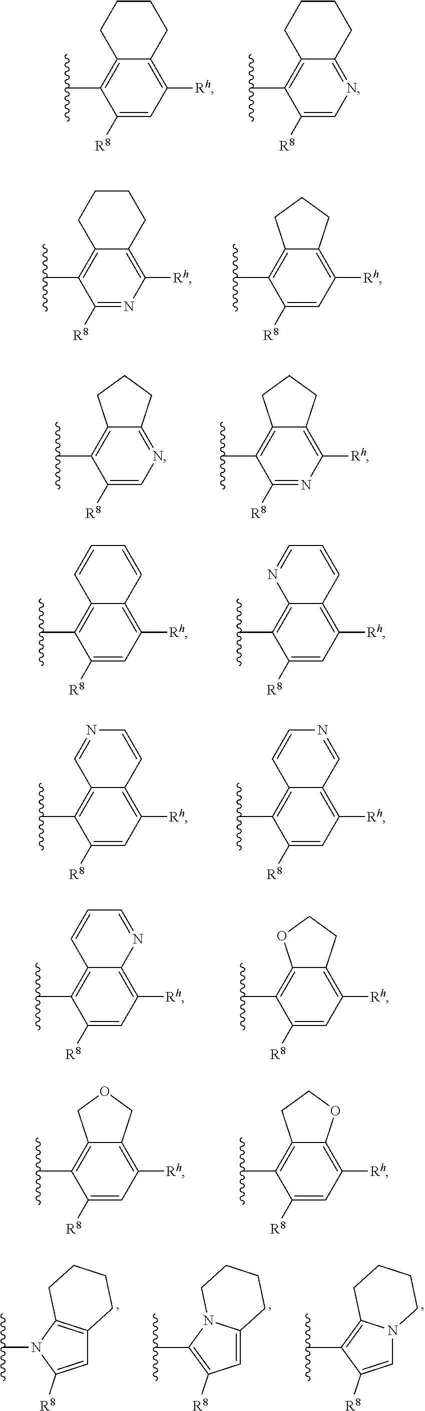

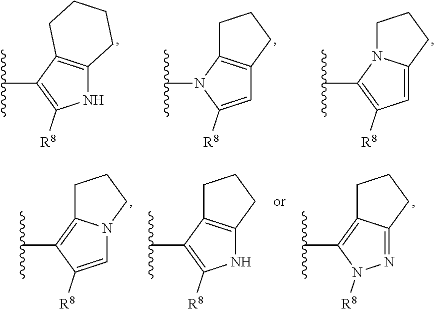

wherein R⁸ is a 5- or 6-membered, optionally substituted heterocyclic or aromatic group, and R^{h} is hydrogen, halo, —OH, —NO₂, —CN, —R^{hh}, —R^{hX}, —OR^{hh}, —COR^{hh}, —COOR^{hh}, —CONH₂, —CONHR^{hh}, —CON(R^{hh})₂, —C(═NH)R^{hh}, —C(═NH)NH₂, —C(═NH)NHR^{hh}, —C(═NH)N(R^{hh})₂, —C(═NR^{hh})R^{hh}, —C(═NR^{hh})NHR^{hh}, —C(═NR^{hh})N(R^{hh})₂, —C(═NOH)R^{hh} or —C(═NOR^{hh})R^{hh}, wherein each —R^{hh} is independently selected from C₁-C₄ alkyl, C₁-C₄ haloalkyl, C₃-C₄ cycloalkyl and C₃-C₄ halocycloalkyl, or any two R^{hh} attached to the same nitrogen atom may, together with the nitrogen atom to which they are attached, form a 3- to 6-membered saturated heterocyclic group, wherein the 3- to 6-membered saturated heterocyclic group is optionally halo substituted, and wherein —R^{hx} is selected from a 3- to 7-membered cyclic group, wherein the 3- to 7-membered cyclic group is optionally halo substituted. In one embodiment, the optional substituents on the heterocyclic or aromatic group of R⁸ are independently selected from halo, —OH, —NH₂, —CN, —NO₂, —B⁷, —CH₂B⁷, —OB⁷, —OCH₂B⁷, —NHB⁷, —N(B⁷)₂, —CONH₂, —CONHB⁷, —CON(B⁷)₂, —NHCOB⁷, —NB⁷COB⁷, or —B⁷⁷—;
wherein each B⁷ is independently selected from a C₁-C₄ alkyl, C₂-C₄ alkenyl, C₂-C₄ alkynyl, C₃-C₆ cycloalkyl or phenyl group, or a 4- to 6-membered heterocyclic group containing one or two ring heteroatoms N and/or O, or two B⁷ together with the nitrogen atom to which they are attached may form a 4- to 6-membered heterocyclic group containing one or two ring heteroatoms N and/or O, wherein any B⁷ may optionally be halo-substituted and/or substituted with one or two substituents independently selected from —OH, —NH₂, —B⁷⁸, —OB⁷⁸, —NHB⁷⁸ or —N(B⁷⁸)₂;
wherein each B⁷⁷ is independently selected from a C₁-C₈ alkylene or C₂-C₈ alkenylene group, wherein one or two carbon atoms in the backbone of the alkylene or alkenylene group may optionally be replaced by one or two heteroatoms N and/or O, and wherein the alkylene or alkenylene group may optionally be halo-substituted and/or substituted with one or two substituents independently selected from —OH, —NH₂, —B⁷⁸, —OB⁷⁸, —NHB⁷⁸ or —N(B⁷⁸)₂; and
wherein each B⁷⁸ is independently selected from a C₁-C₃ alkyl or C₁-C₃ haloalkyl group.

Typically, any divalent group —B⁷⁷— forms a 4- to 6-membered fused ring. Typically, R^{h} is hydrogen, halo, —CN, —R^{hh}, —R^{hx}, —OR^{hh}, —COR^{hh}, —C(═NOH)R^{hh} or —C(═NOR^{hh})R^{hh}, wherein each —R^{hh} is independently selected from C₁-C₃ alkyl, C₁-C₃ fluoroalkyl, cyclopropyl or fluorocyclopropyl, and wherein —R^{hx} is selected from a phenyl, halophenyl or a 5- or 6-membered heteroaryl group, wherein the 5- or 6-membered heteroaryl group is optionally halo substituted. More typically, R^{h} is hydrogen or halo. Typically, the optional substituents on the heterocyclic or aromatic group of R⁸ are independently selected from halo, —OH, —NH₂, —CN, —NO₂, —B⁷, —OB⁷, —NHB⁷ or —N(B⁷)₂, wherein each B⁷ is independently selected from a C₁-C₄ alkyl, C₂-C₄ alkenyl or C₂-C₄ alkynyl group all of which may optionally be halo-substituted.

In one embodiment, —R² has a formula selected from:

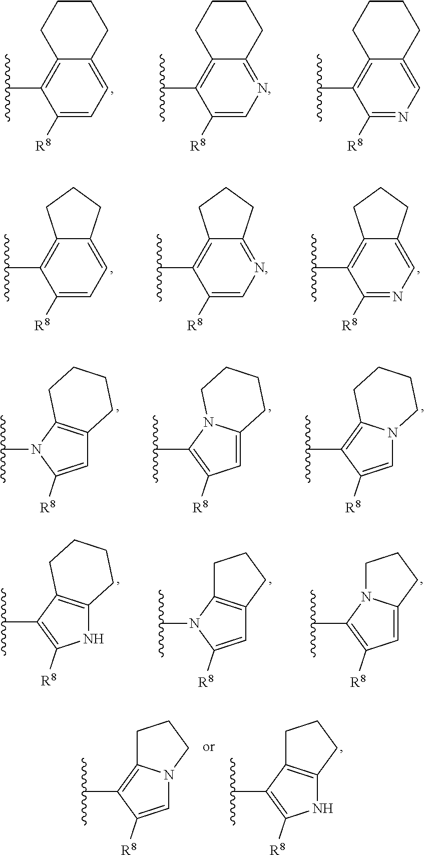

wherein R⁸ is a 5- or 6-membered, optionally substituted heterocyclic or aromatic group. In one embodiment, the optional substituents on the heterocyclic or aromatic group of R⁸ are independently selected from halo, —OH, —NH₂, —CN, —NO₂, —B⁸, —CH₂B⁸, —OB⁸, —OCH₂B⁸, —NHB⁸, —N(B⁸)₂, —CONH₂, —CONHB⁸, —CON(B⁸)₂, —NHCOB⁸, —NB⁸COB⁸, or —B⁸⁸—;
wherein each B⁸ is independently selected from a C₁-C₄ alkyl, C₂-C₄ alkenyl, C₂-C₄ alkynyl, C₃-C₆ cycloalkyl or phenyl group, or a 4- to 6-membered heterocyclic group containing one or two ring heteroatoms N and/or O, or two B⁸ together with the nitrogen atom to which they are attached may form a 4- to 6-membered heterocyclic group containing one or two ring heteroatoms N and/or O, wherein any B⁸ may optionally be halo-substituted and/or substituted with one or two substituents independently selected from —OH, —NH₂, —B⁸⁹, —OB⁸⁹, —NHB⁸⁹ or —N(B⁸⁹)₂;
wherein each B⁸⁸ is independently selected from a C₁-C₈ alkylene or C₂-C₈ alkenylene group, wherein one or two carbon atoms in the backbone of the alkylene or alkenylene group may optionally be replaced by one or two heteroatoms N and/or O, and wherein the alkylene or alkenylene group may optionally be halo-substituted and/or substituted with one or two substituents independently selected from —OH, —NH₂, —B⁸⁹, —OB⁸⁹, —NHB⁸⁹ or —N(B⁸⁹)₂; and
wherein each B⁸⁹ is independently selected from a C₁-C₃ alkyl or C₁-C₃ haloalkyl group.

Typically, any divalent group —B⁸⁸— forms a 4- to 6-membered fused ring. Typically, the optional substituents on the heterocyclic or aromatic group are independently selected from halo, —OH, —NH₂, —CN, —NO₂, —B⁸, —OB⁸, —NHB⁸ or —N(B⁸)₂, wherein each B⁸ is independently selected from a C₁-C₄ alkyl, C₂-C₄ alkenyl or C₂-C₄ alkynyl group all of which may optionally be halo-substituted.

Typically, —R² has a formula selected from:

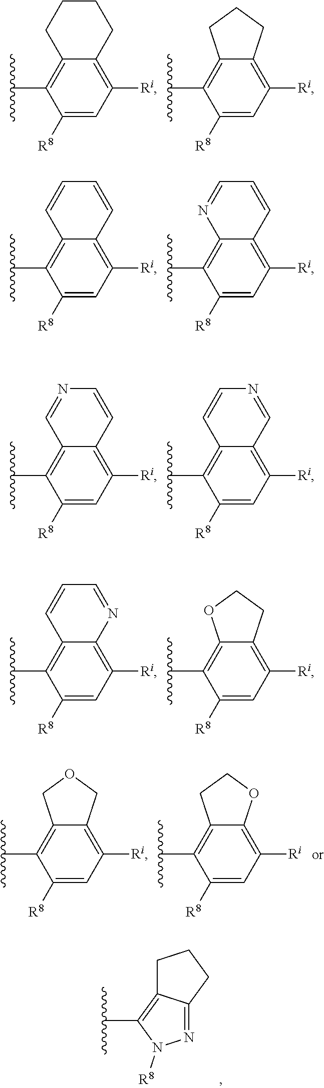

wherein R⁸ is a 5- or 6-membered, optionally substituted heterocyclic or aromatic group, and Rⁱ is hydrogen, halo, —OH, —NO₂, —CN, —Rⁱⁱ, —R^{ix}, —ORⁱⁱ, —CORⁱⁱ, —COORⁱⁱ, —CONH₂, —CONHRⁱⁱ, —CON(Rⁱⁱ)₂, —C(═NH)Rⁱⁱ, —C(═NH)NH₂, —C(═NH)NHRⁱⁱ, —C(═NH)N(Rⁱⁱ)₂, —C(═NRⁱⁱ)Rⁱⁱ, —C(═NRⁱⁱ)NHRⁱⁱ, —C(═NRⁱⁱ)N(Rⁱⁱ)₂, —C(═NOH)Rⁱⁱ or —C(═NORⁱⁱ)Rⁱⁱ, wherein each —Rⁱⁱ is independently selected from C₁-C₄ alkyl, C₁-C₄ haloalkyl, C₃-C₄ cycloalkyl and C₃-C₄ halocycloalkyl, or any two Rⁱⁱ attached to the same nitrogen atom may, together with the nitrogen atom to which they are attached, form a 3- to 6-membered saturated heterocyclic group, wherein the 3- to 6-membered saturated heterocyclic group is optionally halo substituted, and wherein —R^{ix} is selected from a 3- to 7-membered cyclic group, wherein the 3- to 7-membered cyclic group is optionally halo substituted. In one embodiment, the optional substituents on the heterocyclic or aromatic group of R⁸ are independently selected from halo, —OH, —NH₂, —CN, —NO₂, —B⁹, —CH₂B⁹, —OB⁹, —OCH₂B⁹, —NHB⁹, —N(B⁹)₂, —CONH₂, —CONHB⁹, —CON(B⁹)₂, —NHCOB⁹, —NB⁹COB⁹, or —B⁹⁹—;
wherein each B⁹ is independently selected from a C₁-C₄ alkyl, C₂-C₄ alkenyl, C₂-C₄ alkynyl, C₃-C₆ cycloalkyl or phenyl group, or a 4- to 6-membered heterocyclic group containing one or two ring heteroatoms N and/or O, or two B⁹ together with the nitrogen atom to which they are attached may form a 4- to 6-membered heterocyclic group containing one or two ring heteroatoms N and/or O, wherein any B⁹ may optionally be halo-substituted and/or substituted with one or two substituents independently selected from —OH, —NH₂, —B⁹⁸, —OB⁹⁸, —NHB⁹⁸ or —N(B⁹⁸)
wherein each B⁹⁹ is independently selected from a C₁-C₅ alkylene or C₂-C₅ alkenylene group, wherein one or two carbon atoms in the backbone of the alkylene or alkenylene group may optionally be replaced by one or two heteroatoms N and/or O, and wherein the alkylene or alkenylene group may optionally be halo-substituted and/or substituted with one or two substituents independently selected from —OH, —NH₂, —B⁹⁸, —OB⁹⁸, —NHB⁹⁸ or —N(B⁹⁸)₂; and
wherein each B⁹⁸ is independently selected from a C₁-C₃ alkyl or C₁-C₃ haloalkyl group.

Typically, any divalent group —B⁹⁹— forms a 4- to 6-membered fused ring. Typically, Rⁱ is hydrogen, halo, —CN, C₁-C₃ alkyl, C₁-C₃ haloalkyl, cyclopropyl or halocyclopropyl. Typically, Rⁱ is hydrogen, halo, —CN, —Rⁱⁱ, —R^{ix}, —ORⁱⁱ, —CORⁱⁱ, —C(═NOH)Rⁱⁱ or —C(═NORⁱⁱ)Rⁱⁱ, wherein each —Rⁱⁱ is independently selected from C₁-C₃ alkyl, C₁-C₃ fluoroalkyl, cyclopropyl or fluorocyclopropyl, and wherein —R^{ix} is selected from a phenyl, halophenyl or a 5- or 6-membered heteroaryl group, wherein the 5- or 6-membered heteroaryl group is optionally halo substituted. More typically, Rⁱ is hydrogen or halo. Typically, the optional substituents on the heterocyclic or aromatic group of R⁸ are independently selected from halo, —OH, —NH₂, —CN, —NO₂, —B⁹, —OB⁹, —NHB⁹ or —N(B⁹)₂, wherein each B⁹ is independently selected from a C₁-C₄ alkyl, C₂-C₄ alkenyl or C₂-C₄ alkynyl group all of which may optionally be halo-substituted.

In one embodiment, R² is phenyl or a 5- or 6-membered heteroaryl group (such as phenyl, pyridinyl, pyridazinyl, pyrimidinyl, pyrazinyl, pyrrolyl, pyrazolyl or imidazolyl); wherein:
(i) the phenyl or 5- or 6-membered heteroaryl group is substituted at the α position with a substituent selected from —R⁴, —OR⁴ and —COR⁴, wherein R⁴ is selected from a C₁-C₆ alkyl, C₂-C₆ alkenyl, C₂-C₆ alkynyl or C₂-C₆ cyclic group and wherein R⁴ is optionally substituted with one or more halo groups; and
optionally the phenyl or 5- or 6-membered heteroaryl group is further substituted at the α′ position with a substituent selected from —R¹⁴, —OR¹⁴ and —COR¹⁴, wherein R¹⁴ is selected from a C₁-C₆ alkyl, C₂-C₆ alkenyl, C₂-C₆ alkynyl or C₂-C₆ cyclic group and wherein R¹⁴ is optionally substituted with one or more halo groups; and
optionally the phenyl or 5- or 6-membered heteroaryl group is further substituted (typically with one, two or three substituents independently selected from halo, —NO₂, —CN, C₁-C₄ alkyl, C₁-C₄ haloalkyl, a 3- to 5-membered cyclic group (such as a 5-membered heteroaryl group), a 3- to 5-membered halocyclic group (such as a 5-membered haloheteroaryl group), —COR¹⁵, —COOR¹⁵, —CONH₂, —CONHR¹⁵, —CON(R¹⁵)₂, —C(═NOH)R¹⁵ or —C(═NOR¹⁵)R¹⁵, wherein each —R¹⁵ is independently selected from a C₁-C₄ alkyl or C₁-C₄ haloalkyl group); or
(ii) the phenyl or 5- or 6-membered heteroaryl group is substituted with a cycloalkyl, cycloalkenyl, non-aromatic heterocyclic, aryl or heteroaryl ring which is fused to the parent phenyl or 5- or 6-membered heteroaryl group across the α,β positions and which is optionally substituted with one or more halo groups; and
optionally the phenyl or 5- or 6-membered heteroaryl group is further substituted at the α′ position with a substituent selected from —R⁴, —OR⁴ and —COR⁴, wherein R⁴ is selected from a C₁-C₆ alkyl, C₂-C₆ alkenyl, C₂-C₆ alkynyl or C₂-C₆ cyclic group and wherein R⁴ is optionally substituted with one or more halo groups; and
optionally the phenyl or 5- or 6-membered heteroaryl group is further substituted (typically with one or two substituents independently selected from halo, —NO₂, —CN, C₁-C₄ alkyl, C₁-C₄ haloalkyl, a 3- to 5-membered cyclic group (such as a 5-membered heteroaryl group), a 3- to 5-membered halocyclic group (such as a 5-membered haloheteroaryl group), —COR¹⁵, —COOR¹⁵, —CONH₂, —CONHR¹⁵, —CON(R¹⁵)₂, —C(═NOH)R¹⁵ or —C(═NOR¹⁵)R¹⁵, wherein each —R¹⁵ is independently selected from a C₁-C₄ alkyl or C₁-C₄ haloalkyl group); or
(iii) the phenyl or 5- or 6-membered heteroaryl group is substituted with a first cycloalkyl, cycloalkenyl, non-aromatic heterocyclic, aryl or heteroaryl ring which is fused to the parent phenyl or 5- or 6-membered heteroaryl group across the α,β positions and which is optionally substituted with one or more halo groups; and
the phenyl or 5- or 6-membered heteroaryl group is substituted with a second cycloalkyl, cycloalkenyl, non-aromatic heterocyclic, aryl or heteroaryl ring which is fused to the parent phenyl or 5- or 6-membered heteroaryl group across the α′,β′ positions and which is optionally substituted with one or more halo groups; and
optionally the phenyl group is further substituted (typically with a substituent selected from halo, —NO₂, —CN, C₁-C₄ alkyl, C₁-C₄ haloalkyl, a 3- to 5-membered cyclic group (such as a 5-membered heteroaryl group), a 3- to 5-membered halocyclic group (such as a 5-membered haloheteroaryl group), —COR¹⁵, —COOR¹⁵, —CONH₂, —CONHR¹⁵, —CON(R¹⁵)₂, —C(═NOH)R¹⁵ or —C(═NOR¹⁵)R¹⁵, wherein each —R¹⁵ is independently selected from a C₁-C₄ alkyl or C₁-C₄ haloalkyl group); or
(iv) the phenyl or 5- or 6-membered heteroaryl group is substituted at the α-position with a monovalent heterocyclic group or a monovalent aromatic group selected from phenyl, pyridinyl, pyrimidinyl, pyrazolyl, imidazolyl, triazolyl or tetrahydropyranyl, wherein the monovalent heterocyclic or aromatic group may optionally be substituted with one or two substituents independently selected from halo, —CN, —R¹³, —OR¹³, —N(R¹³)₂, —C≡CR¹³, —R¹²—CN, —R¹²-R¹³, —R¹²—OR¹³, —R¹²—N(R¹³)₂, —R¹²—C≡CR¹³, —O—R¹²—CN, —O—R¹²-R¹³, —O—R¹²—OR¹³, —O—R¹²—N(R¹³)₂ or —O—R¹²—C≡CR¹³, and wherein a ring atom of the monovalent heterocyclic or aromatic group is directly attached to the α-ring atom of the parent phenyl or 5- or 6-membered heteroaryl group; wherein R¹² is independently selected from a C₁-C₃ alkylene or C₁-C₃ haloalkylene group; and R¹³ is independently selected from hydrogen or a C₁-C₄ alkyl, C₁-C₄ haloalkyl or 3- to 6-membered cyclic group (such as a C₃-C₆ cycloalkyl, phenyl, or 4- to 6-membered saturated heterocyclic group), wherein the 3- to 6-membered cyclic group may optionally be substituted with one or more halo, methyl or halomethyl groups; and
optionally the phenyl or 5- or 6-membered heteroaryl group is further substituted at the α′ position with a substituent selected from —R⁴, —OR⁴ and —COR⁴, wherein R⁴ is selected from a C₁-C₆ alkyl, C₂-C₆ alkenyl, C₂-C₆ alkynyl or C₂-C₆ cyclic group and wherein R⁴ is optionally substituted with one or more halo groups; and
optionally the phenyl or 5- or 6-membered heteroaryl group is further substituted (typically with one, two or three substituents independently selected from halo, —NO₂, —CN, C₁-C₄ alkyl, C₁-C₄ haloalkyl, a 3- to 5-membered cyclic group (such as a 5-membered heteroaryl group), a 3- to 5-membered halocyclic group (such as a 5-membered haloheteroaryl group), —COR¹⁵, —COOR¹⁵, —CONH₂, —CONHR¹⁵, —CON(R¹⁵)₂, —C(═NOH)R¹⁵ or —C(═NOR¹⁵)R¹⁵, wherein each —R¹⁵ is independently selected from a C₁-C₄ alkyl or C₁-C₄ haloalkyl group); or
(v) the phenyl or 5- or 6-membered heteroaryl group is substituted at the α-position with a monovalent heterocyclic group or a monovalent aromatic group selected from phenyl, pyridinyl, pyrimidinyl, pyrazolyl, imidazolyl, triazolyl or tetrahydropyranyl, wherein the monovalent heterocyclic or aromatic group may optionally be substituted with one or two substituents independently selected from halo, —CN, —R¹³, —OR¹³, —N(R¹³)₂, —C≡CR¹³, —R¹²—CN, —R¹²-R¹³, —R¹²—OR¹³, —R¹²—N(R¹³)₂, —R¹²—C≡CR¹³, —O—R¹²—CN, —O—R¹²-R¹³, —O—R¹²—OR¹³, —O—R¹²—N(R¹³)₂ or —O—R¹²—C≡CR¹³, and wherein a ring atom of the monovalent heterocyclic or aromatic group is directly attached to the α-ring atom of the parent phenyl or 5- or 6-membered heteroaryl group; wherein R¹² is independently selected from a C₁-C₃ alkylene or C₁-C₃ haloalkylene group; and R¹³ is independently selected from hydrogen or a C₁-C₄ alkyl, C₁-C₄ haloalkyl or 3- to 6-membered cyclic group (such as a C₃_C₆ cycloalkyl, phenyl, or 4- to 6-membered saturated heterocyclic group), wherein the 3- to 6-membered cyclic group may optionally be substituted with one or more halo, methyl or halomethyl groups; and
optionally the phenyl or 5- or 6-membered heteroaryl group is further substituted with a cycloalkyl, cycloalkenyl, non-aromatic heterocyclic, aryl or heteroaryl ring which is fused to the parent phenyl or 5- or 6-membered heteroaryl group across the α′,β′ positions and which is optionally substituted with one or more halo groups; and
optionally the phenyl or 5- or 6-membered heteroaryl group is further substituted (typically with one or two substituents independently selected from halo, —NO₂, —CN, C₁-C₄ alkyl, C₁-C₄ haloalkyl, a 3- to 5-membered cyclic group (such as a 5-membered heteroaryl group), a 3- to 5-membered halocyclic group (such as a 5-membered haloheteroaryl group), —COR¹⁵, —COOR¹⁵, —CONH₂, —CONHR¹⁵, —CON(R¹⁵)₂, —C(═NOH)R¹⁵ or —C(═NOR¹⁵)R¹⁵, wherein each —R¹⁵ is independently selected from a C₁-C₄ alkyl or C₁-C₄ haloalkyl group).

In the embodiment directly above, where a group or moiety is optionally substituted with one or more halo groups, it may be substituted for example with one, two, three, four, five or six halo groups.

In one aspect of any of the above embodiments, R² contains from 10 to 50 atoms other than hydrogen or halogen. More typically, R² contains from 10 to 40 atoms other than hydrogen or halogen. More typically, R² contains from 10 to 35 atoms other than hydrogen or halogen. More typically still, R² contains from 10 to 30 or from 12 to 30 atoms other than hydrogen or halogen. Yet more typically, R² contains from 10 to 25 or from 12 to 25 atoms other than hydrogen or halogen.

In a first specific embodiment of the invention, the compound is a compound of formula (I) wherein:
Q¹ and Q² are both N;
Q³ is NR^{qq};
R^{qq} is independently selected from hydrogen or a C₁-C₄ alkyl or C₃-C₄ cycloalkyl group, wherein the C₁-C₄ alkyl or C₃-C₄ cycloalkyl group may optionally be substituted with one or more fluoro and/or chloro groups;
J is —S—, —SO—, —SO₂—, —SO(═NR^{jj})—, —S—C(R^{j})₂—, —SO—C(R^{j})₂—, —SO₂—C(R^{j})₂—, or —SO(═NR^{jj})—C(R^{j})₂—;
each R^{j} where present is independently selected from hydrogen or a fluoro, chloro, methyl or ethyl group, wherein any methyl or ethyl group may optionally be substituted with one or more fluoro and/or chloro groups, or any two R^{j} attached to the same carbon atom may, together with the carbon atom to which they are attached, form a 3- or 4-membered cycloalkyl group, or form an oxetanyl group, wherein the 3- or 4-membered cycloalkyl group or the oxetanyl group may optionally be substituted with one or more fluoro and/or chloro groups;
each R^{jj} where present is selected from hydrogen, —CN, or a C₁-C₄ alkyl or C₃-C₄ cycloalkyl group, wherein the C₁-C₄ alkyl or C₃-C₄ cycloalkyl group may optionally be substituted with one or more fluoro and/or chloro groups;
a carbon or nitrogen atom of R¹ is directly attached to the sulfur atom of J;
R¹ is a saturated or unsaturated C₁-C₂₀ hydrocarbyl group, wherein the hydrocarbyl group may be straight-chained or branched, or be or include cyclic groups, wherein the hydrocarbyl group may optionally be substituted, wherein the hydrocarbyl group may optionally include one or more heteroatoms N, O or S in its carbon skeleton, and wherein R¹ contains from 1 to 30 atoms other than hydrogen or halogen;
G is —O—, —C(R^{g})₂—, or —NR^{gg}—;
each R^{g} where present is independently selected from hydrogen or a fluoro, chloro, methyl or ethyl group, wherein any methyl or ethyl group may optionally be substituted with one or more fluoro and/or chloro groups, or any two R^{g} attached to the same carbon atom may, together with the carbon atom to which they are attached, form a 3- or 4-membered cycloalkyl group, or form an oxetanyl group, wherein the 3- or 4-membered cycloalkyl group or the oxetanyl group may optionally be substituted with one or more fluoro and/or chloro groups;
each R^{gg} where present is selected from hydrogen or a C₁-C₄ alkyl or C₃-C₄ cycloalkyl group, wherein the C₁-C₄ alkyl or C₃-C₄ cycloalkyl group may optionally be substituted with one or more fluoro and/or chloro groups; and
R² is a phenyl or a 5- or 6-membered heteroaryl group, wherein the phenyl or the heteroaryl group is substituted at the α-position, wherein R² may optionally be further substituted, and wherein R² contains from 10 to 35 atoms other than hydrogen or halogen.

Typically in accordance with the first specific embodiment, J is —SO—, —SO₂—, —SO(═NR^{jj})—, —SO—C(R^{j})₂—, —SO₂—C(R^{j})₂—, or —SO(═NR^{jj})—C(R^{j})₂—. More typically, J is —SO—, —SO₂—, —SO—CH₂— or —SO₂—CH₂—. More typically still, J is —SO— or—SO₂—.

Typically in accordance with the first specific embodiment, at least one substituent at the α and/or α′ positions of the phenyl or the heteroaryl group comprises a carbon atom. Typically, R² is a phenyl or a 5- or 6-membered heteroaryl group, wherein the phenyl or the heteroaryl group is substituted at the α and α′ positions, and wherein R² may optionally be further substituted.

In a first aspect of the first specific embodiment, Q³ is NH.

In a second aspect of the first specific embodiment, G is —O— or —NR^{gg}—.

In a third aspect of the first specific embodiment, R² is a phenyl or a 5- or 6-membered heteroaryl group, wherein the phenyl or the heteroaryl group is substituted at the α and α′ positions, wherein both substituents at the α and α′ positions comprise a carbon atom, wherein R² may optionally be further substituted, and wherein R² contains from 10 to 35 atoms other than hydrogen or halogen.

In a second specific embodiment of the invention, the compound is a compound of formula (I) wherein:
Q¹ and Q² are both N;
Q³ is NH;
J is —SO— or —SO₂—;
a carbon or nitrogen atom of R¹ is directly attached to the sulfur atom of J;
R¹ is a saturated or unsaturated C₁-C₂₀ hydrocarbyl group, wherein the hydrocarbyl group may be straight-chained or branched, or be or include cyclic groups, wherein the hydrocarbyl group may optionally be substituted, wherein the hydrocarbyl group may optionally include one or more heteroatoms N, O or S in its carbon skeleton, and wherein R¹ contains from 1 to 30 atoms other than hydrogen or halogen;
G is —O—, —CH₂—, or —NH—; and
R² is a phenyl or a 5- or 6-membered heteroaryl group, wherein the phenyl or the heteroaryl group is substituted at the α-position, wherein at least one substituent at the α and/or α′ positions comprises a carbon atom, wherein R² may optionally be further substituted, and wherein R² contains from 10 to 35 atoms other than hydrogen or halogen.

Typically in accordance with the second specific embodiment, R² is a phenyl or a 5- or 6-membered heteroaryl group, wherein the phenyl or the heteroaryl group is substituted at the α and α′ positions, and wherein R² may optionally be further substituted. Typically both substituents at the α and α′ positions comprise a carbon atom.

Typically in accordance with either of the first or second specific embodiments:
R¹ is a C₁-C₁₅ alkyl, C₂-C₁₅ alkenyl or C₂-C₁₅ alkynyl group, all of which may optionally include one, two or three heteroatoms N, O or S in their carbon skeleton; or
R¹ is a 3- to 12-membered cyclic group; or
R¹ is R¹⁰-L-, wherein R¹⁰ is a 3- to 12-membered cyclic group, wherein L is —NH— or an alkylene group, wherein the alkylene group may optionally include one or two heteroatoms independently selected from oxygen and nitrogen in its carbon skeleton, wherein the alkylene group may optionally be substituted with one or more substituents independently selected from halo, —CN, —OH, —NH₂ and oxo (═O), and wherein L contains from 1 to 10 atoms other than hydrogen or halogen;
wherein any C₁-C₁₅ alkyl, C₂-C₁₅ alkenyl, C₂-C₁₅ alkynyl or 3- to 12-membered cyclic group of R¹ or R¹⁰ may optionally be substituted with one or more halo groups, and/or with one, two or three substituents independently selected from C₁-C₅ alkyl, C₁-C₅ haloalkyl, C₃-C₆ cycloalkyl, C₃-C₆ halocycloalkyl, C₂-C₅ alkenyl, C₂-C₅ haloalkenyl, C₅-C₆ cycloalkenyl, C₅-C₆ halocycloalkenyl, C₂-C₅ alkynyl, C₂-C₅ haloalkynyl, phenyl, halophenyl, 5- or 6-membered heteroaryl (optionally halo substituted), —R¹¹—CN, —R¹¹—N₃, —R¹¹—NO₂, —R¹¹—N(R¹²)₂, —R¹¹—OR¹², —R¹¹—COR¹², —R¹¹—COOR¹², —R¹¹—CON(R¹²)₂, —R¹¹—SO₂R¹², —R¹¹—SO₂N(R¹²)₂, oxo (═O),

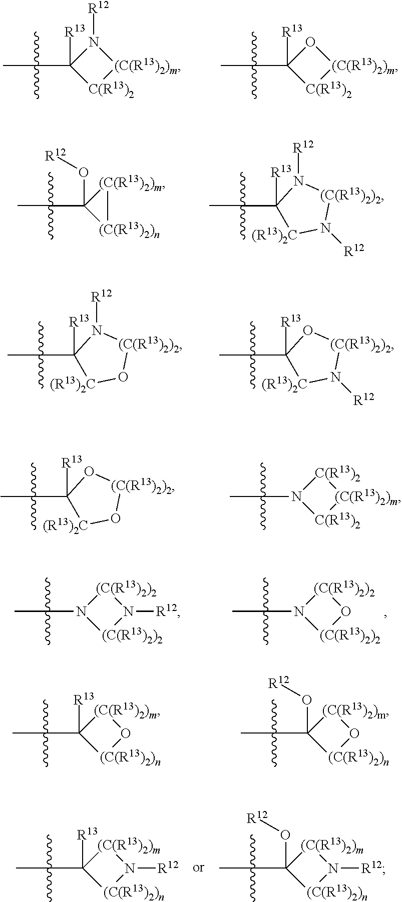

wherein R¹¹ is independently selected from a bond, C₁-C₄ alkylene, C₁-C₄ haloalkylene, C₃-C₄ cycloalkylene or C₃-C₄ halocycloalkylene;
each R¹² is independently selected from hydrogen, C₁-C₅ alkyl, C₁-C₅ haloalkyl, C₃-C₅ cycloalkyl or C₃-C₅ halocycloalkyl, or any two R¹² attached to the same nitrogen atom may together form a C₂-C₅ alkylene or C₂-C₅ haloalkylene group;
each R¹³ is independently selected from hydrogen or halo;
m is 1, 2 or 3; and
n is 1, 2 or 3.

More typically, in accordance with the second specific embodiment:
R¹ is a C₁-C₁₀ alkyl group, wherein the C₁-C₁₀ alkyl group may optionally include one, two or three heteroatoms independently selected from oxygen and nitrogen in its carbon skeleton; or
R¹ is a phenyl or a 5- or 6-membered heteroaryl group; or
R¹ is a 8- to 10-membered fused bicyclic group, wherein a first ring in the fused bicyclic structure is a non-aromatic ring and a second ring in the fused bicyclic structure is an aromatic ring; or
R¹ is a 3- to 7-membered non-aromatic monocyclic group or a 7- to 12-membered non-aromatic bicyclic group; or
R¹ is R¹⁰-L-, wherein:
L is —NH— or an alkylene group, wherein the alkylene group may optionally include a single nitrogen atom in its carbon skeleton, wherein the alkylene group may optionally be substituted with one or more fluoro groups, and wherein L contains from 1 to 6 atoms other than hydrogen or halogen; and
R¹⁰ is a phenyl or a 5- or 6-membered heteroaryl group; or
R¹⁰ is a 3- to 7-membered non-aromatic monocyclic group;
wherein any C₁-C₁₀ alkyl, phenyl, 5- or 6-membered heteroaryl, 8- to 10-membered fused bicyclic, 3- to 7-membered non-aromatic monocyclic or 7- to 12-membered non-aromatic bicyclic group of R¹ or R¹⁰ may optionally be substituted with one or more halo groups, and/or with one or two substituents independently selected from C₁-C₅ alkyl, C₁-C₅ haloalkyl, C₃-C₆ cycloalkyl, C₃-C₆ halocycloalkyl, phenyl, halophenyl, 5- or 6-membered heteroaryl (optionally halo substituted), —R¹¹—CN, —R¹¹—N(R¹²)₂, —R¹¹—OR¹², —R¹¹—COR¹², —R¹¹—COOR¹², —R¹¹—CON(R¹²)₂, oxo (═O),

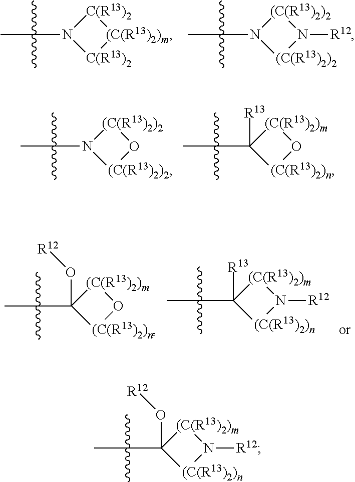

wherein R¹¹ is independently selected from a bond, C₁-C₄ alkylene or C₁-C₄ haloalkylene; each R¹² is independently selected from hydrogen, C₁-C₅ alkyl, C₁-C₅ haloalkyl, C₃-C₅ cycloalkyl or C₃-C₅ halocycloalkyl, or any two R¹² attached to the same nitrogen atom may together form a C₂-C₅ alkylene or C₂-C₅ haloalkylene group; each R¹³ is independently selected from hydrogen or halo; m is 1 or 2; and n is 1 or 2.

Typically in accordance with either of the first or second specific embodiments, R² has a formula selected from:

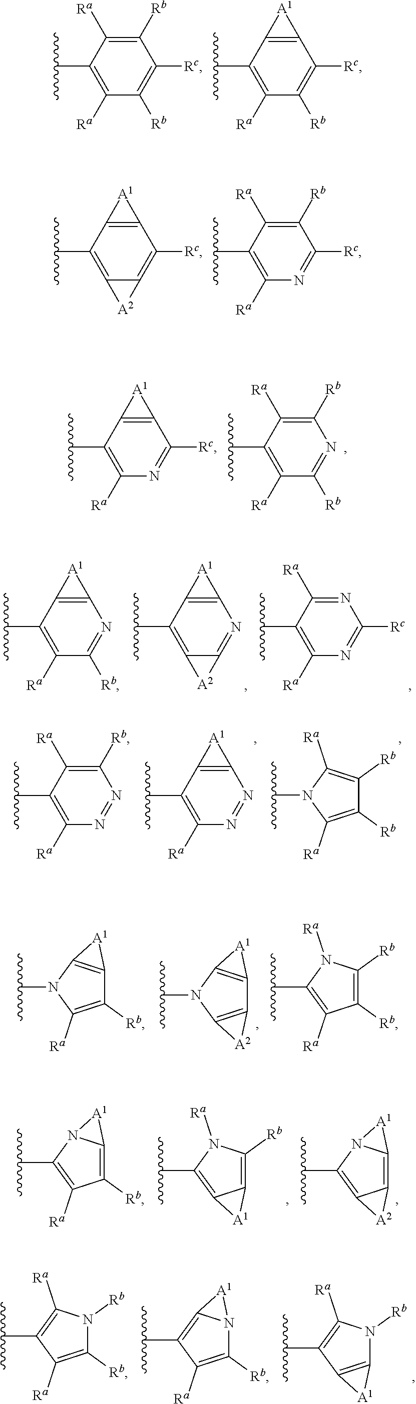

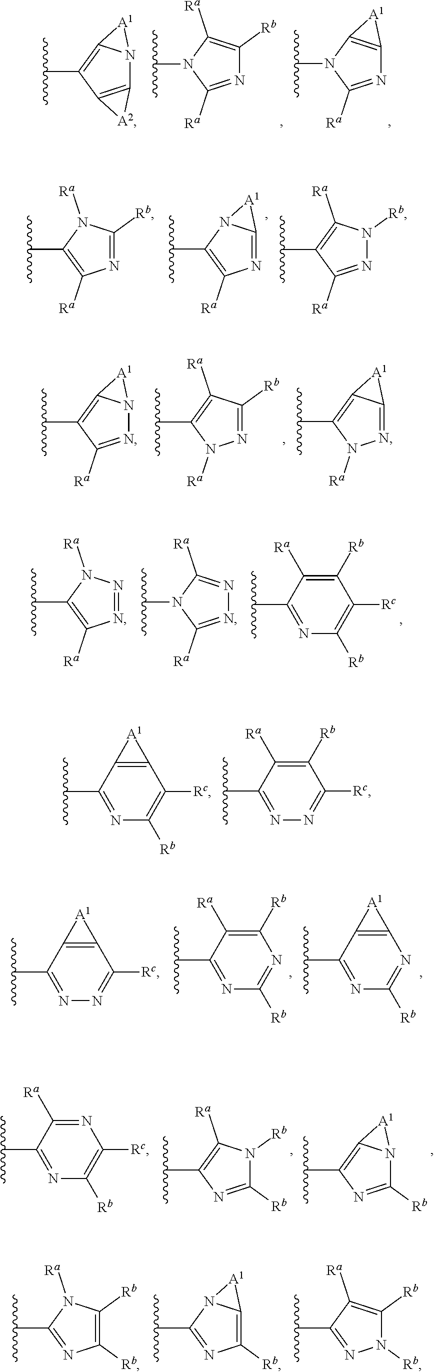

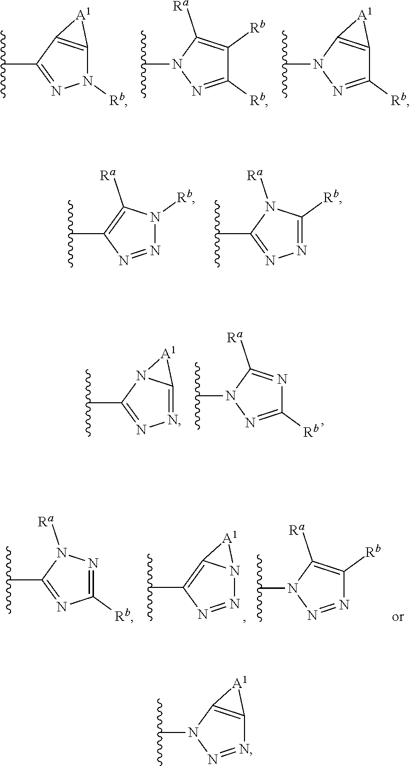

wherein:
A¹ and A² are each independently selected from a straight-chained alkylene group or a straight-chained alkenylene group, wherein one or two carbon atoms in the backbone of the alkylene or alkenylene group may optionally be replaced by one or two heteroatoms independently selected from nitrogen and oxygen, wherein any ring containing A¹ or A² is a 5- or 6-membered ring, and wherein the alkylene or alkenylene group may optionally be substituted with one or more substituents independently selected from halo, —OH, —CN, —NO₂, C₁-C₄ alkyl, C₁-C₄ haloalkyl, —O(C₁-C₄ alkyl) or —O(C₁-C₄ haloalkyl);
each R^{a} is independently selected from hydrogen, halo, —R^{aa}, —OR^{aa} or —COR^{aa}, provided that at least one R^{a} is —R^{aa}, —OR^{aa} or —COR^{aa};
each R^{b} is independently selected from hydrogen, halo, —NO₂, —CN, —R^{aa}, —OR^{aa} or —COR^{aa};
provided that any R^{a} or R^{b} that is directly attached to a ring nitrogen atom is not halo, —NO₂, —CN, or —OR^{aa};
each R^{aa} is independently selected from a C₁-C₆ alkyl, C₂-C₆ alkenyl, C₂-C₆ alkynyl or a 3- to 7-membered cyclic group, wherein each C₁-C₆ alkyl, C₂-C₆ alkenyl, or C₂-C₆ alkynyl group is optionally substituted with one or more substituents independently selected from halo, —OH, —CN, —NO₂, —O(C₁-C₄ alkyl) or —O(C₁-C₄ haloalkyl), and wherein each 3- to 7-membered cyclic group is optionally substituted with one or more substituents independently selected from halo, —OH, —NH₂, —CN, —NO₂, —B¹, —CH₂B¹, —OB¹, —OCH₂B¹, —NHB¹, —N(B¹)₂, —CONH₂, —CONHB¹, —CON(B¹)₂, —NHCOB¹, —NB¹COB¹, or —B¹¹—;
each B¹ is independently selected from a C₁-C₆ alkyl, C₂-C₆ alkenyl, C₂-C₆ alkynyl, C₃-C₁₀ cycloalkyl, C₅-C₁₀ cycloalkenyl, C₆-C₁₀ aryl, or a 4- to 10-membered heterocyclic group containing one or two ring heteroatoms N and/or O, or two B¹ together with the nitrogen atom to which they are attached may form a 4- to 10-membered heterocyclic group containing one or two ring heteroatoms N and/or O, wherein any B¹ may optionally be halo-substituted and/or substituted with one or two substituents independently selected from —OH, —NH₂, —B¹², —OB¹², —NHB¹² or —N(B¹²)₂;
each B¹¹ is independently selected from a C₁-C₈ alkylene or C₂-C₈ alkenylene group, wherein one or two carbon atoms in the backbone of the alkylene or alkenylene group may optionally be replaced by one or two heteroatoms N and/or O, and wherein the alkylene or alkenylene group may optionally be halo-substituted and/or substituted with one or two substituents independently selected from —OH, —NH₂, —B¹², —OB¹², —NHB¹² or —N(B¹²)₂;
each B¹² is independently selected from a C₁-C₃ alkyl or C₁-C₃ haloalkyl group;
each R^{c} is selected from hydrogen, halo, —OH, —NO₂, —CN, —R^{cc}—, —R^{cx}, —OR^{cc}, —COR^{cc}, —COOR^{cc}, —CONH₂, —CONHR^{cc}, —CON(R^{cc})₂, —C(═NH)R^{cc}—, —C(═NH)NH₂, —C(═NH)NHR^{cc}, —C(═NH)N(R^{cc})₂, —C(═NR^{cc})R^{cc}, —C(═NR^{cc})NHR^{cc}, —C(═NR^{cc})N(R^{cc})₂, —C(═NOH)R^{cc} or —C(═NOR^{cc})R^{cc};
each R^{cc} is independently selected from C₁-C₄ alkyl, C₁-C₄ haloalkyl, C₃-C₄ cycloalkyl or C₃-C₄ halocycloalkyl, or any two R^{cc} attached to the same nitrogen atom may, together with the nitrogen atom to which they are attached, form a 3- to 6-membered saturated heterocyclic group, wherein the 3- to 6-membered saturated heterocyclic group is optionally halo substituted; and
each R^{cx} is selected from a 3- to 7-membered cyclic group, wherein the 3- to 7-membered cyclic group is optionally halo substituted.

In one aspect of the second specific embodiment:
A¹ and A² are each independently selected from a straight-chained alkylene group, wherein one carbon atom in the backbone of the alkylene group may optionally be replaced by an oxygen atom, wherein any ring containing A¹ or A² is a 5- or 6-membered ring, and wherein the alkylene group may optionally be substituted with one or more fluoro and/or chloro groups;
each R^{a} is independently selected from hydrogen, halo or —R^{aa}, provided that at least one R^{a} is —R^{aa};
each R^{b} is independently selected from hydrogen, halo, methyl or fluoromethyl; provided that any R^{a} or R^{b} that is directly attached to a ring nitrogen atom is not halo;
each R^{aa} is independently selected from a C₁-C₄ alkyl or a 3- to 6-membered cyclic group, wherein each C₁-C₄ alkyl group is optionally substituted with one or more halo substituents and/or one or two substituents independently selected from —OH, —CN, —O(C₁-C₄ alkyl) or —O(C₁-C₄ haloalkyl), and wherein each 3- to 6-membered cyclic group is optionally substituted with one or more halo substituents and/or one or two substituents independently selected from halo, —OH, —CN, —B¹, —CH₂B¹, —OB¹ or —OCH₂B¹;
each B¹ is independently selected from a C₁-C₄ alkyl, C₃-C₆ cycloalkyl or phenyl group, or a 4- to 6-membered heterocyclic group containing one or two ring heteroatoms N and/or O, wherein any B may optionally be halo-substituted and/or substituted with one or two substituents independently selected from —OH, —B¹² or —OB¹²;
each B¹² is independently selected from a C₁-C₃ alkyl or C₁-C₃ haloalkyl group;
each R^{c} is selected from hydrogen, halo, —CN, —R^{cc}—, —R^{cx}—, —OR^{cc}, —COR^{cc}, —C(═NOH)R^{cc} or —C(═NOR^{cc})R^{cc};
each R^{cc} is independently selected from C₁-C₃ alkyl, C₁-C₃ fluoroalkyl, cyclopropyl or fluorocyclopropyl; and
each R^{cx} is selected from a phenyl, halophenyl or a 5- or 6-membered heteroaryl group, wherein the 5- or 6-membered heteroaryl group is optionally halo substituted. Typically, in the aspect of the second specific embodiment immediately above, R² has a formula selected from:

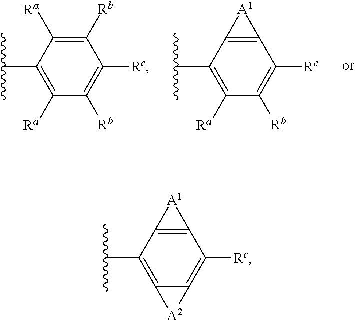

and each R^{a} is —R^{aa}.

In one aspect of any of the above embodiments, the compound of formula (I) has a molecular weight of from 250 to 2000 Da. Typically, the compound of formula (I) has a molecular weight of from 280 to 900 Da. More typically, the compound of formula (I) has a molecular weight of from 300 to 600 Da.

A second aspect of the invention provides a compound selected from the group consisting of:

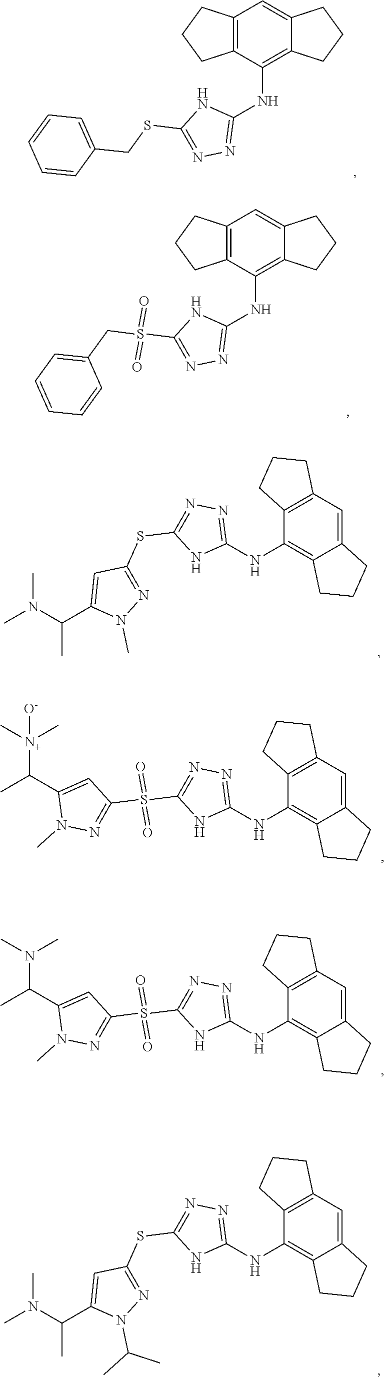 [SMILES: c1ccc(CSc2nnc(Nc3c4c(cc5c3CCC5)CCC4)[nH]2)cc1 ; O=S(=O)(Cc1ccccc1)c1nnc(Nc2c3c(cc4c2CCC4)CCC3)[nH]1 ; CC(c1cc(Sc2nnc(Nc3c4c(cc5c3CCC5)CCC4)[nH]2)nn1C)N(C)C ; CC(c1cc(S(=O)(=O)c2nnc(Nc3c4c(cc5c3CCC5)CCC4)[nH]2)nn1C)[N+](C)(C)[O-] ; CC(c1cc(S(=O)(=O)c2nnc(Nc3c4c(cc5c3CCC5)CCC4)[nH]2)nn1C)N(C)C ; CC(c1cc(Sc2nnc(Nc3c4c(cc5c3CCC5)CCC4)[nH]2)nn1C(C)C)N(C)C]

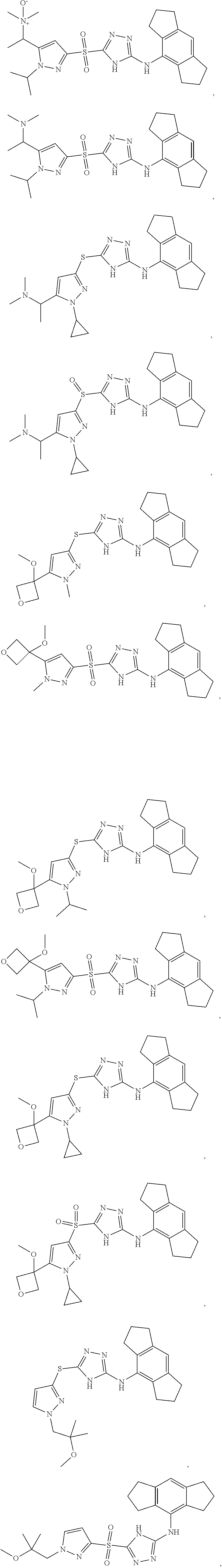

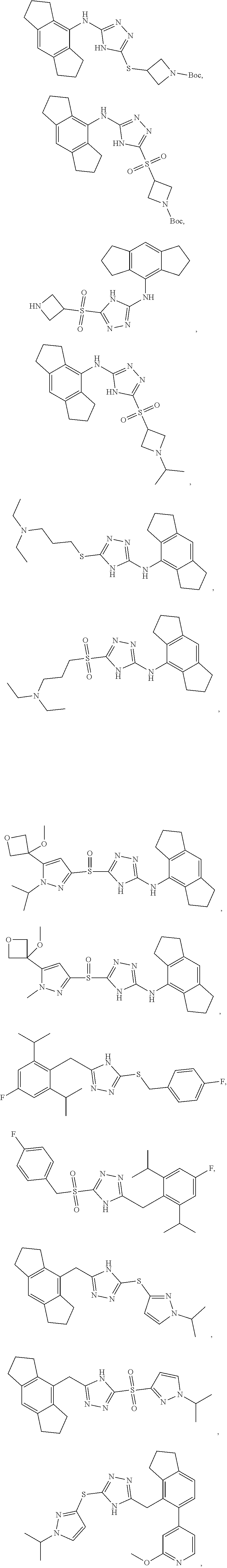

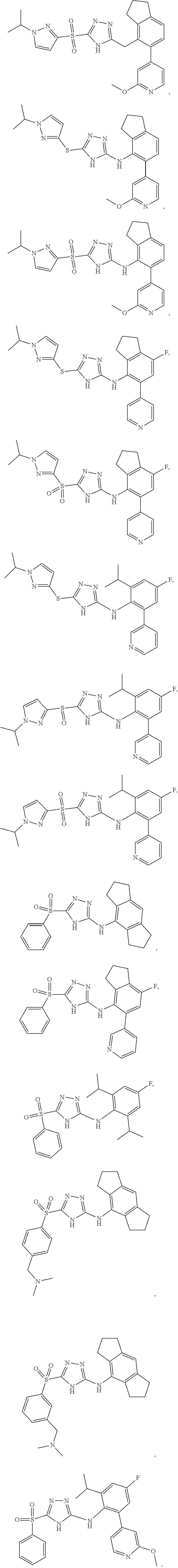

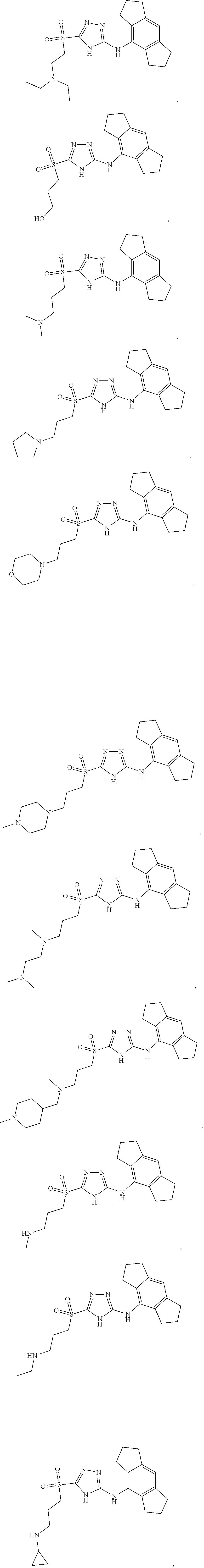

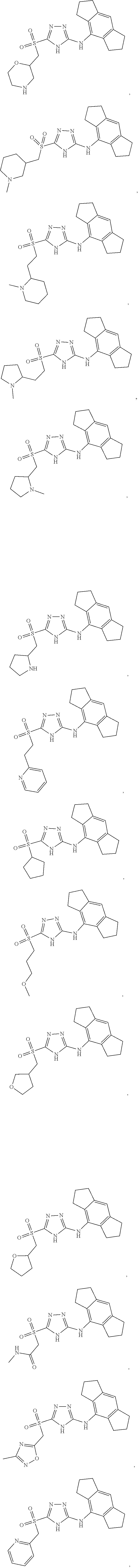

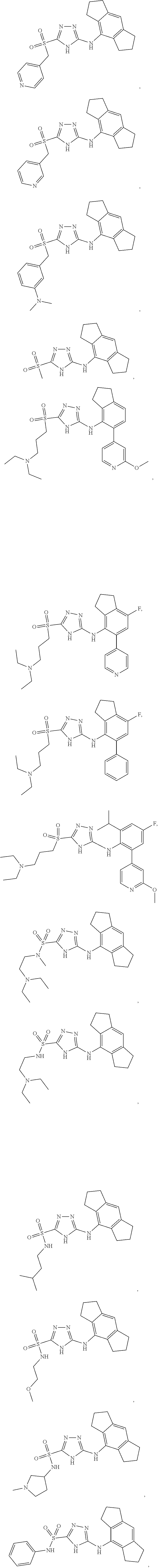

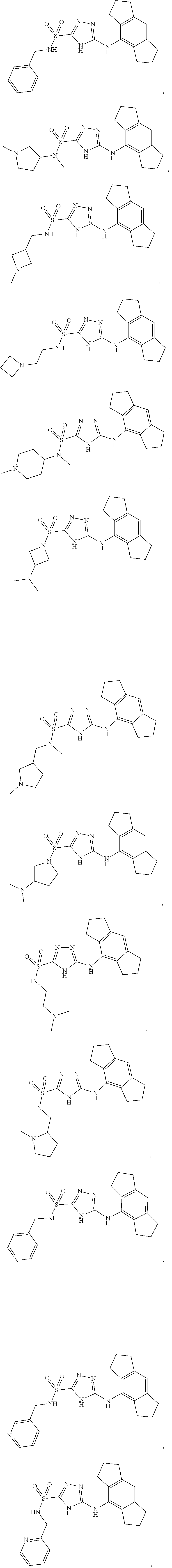

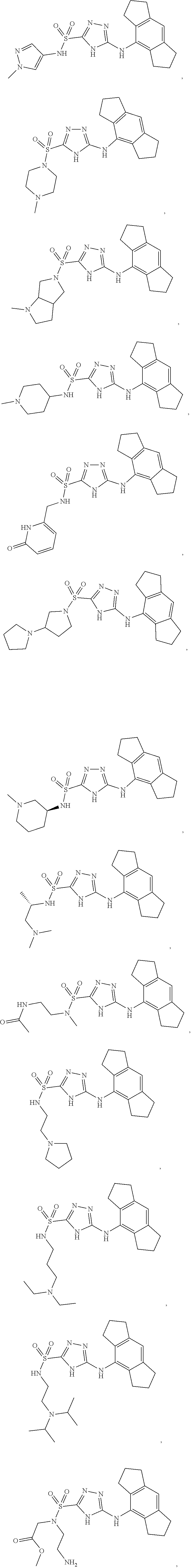

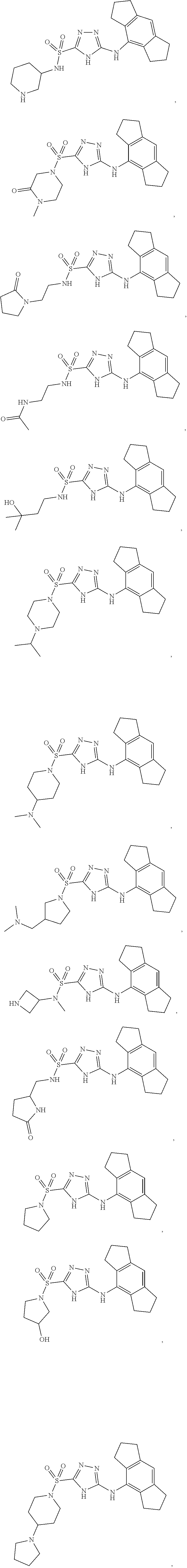

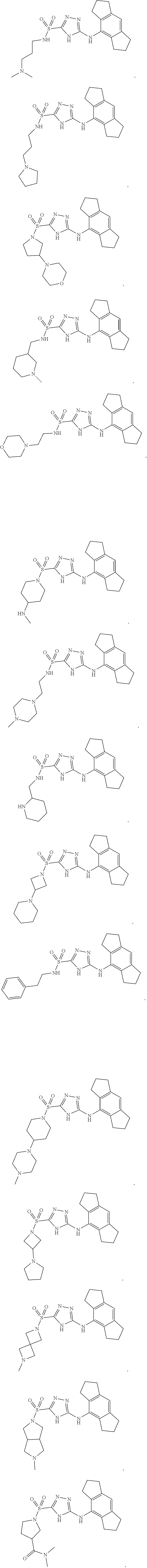

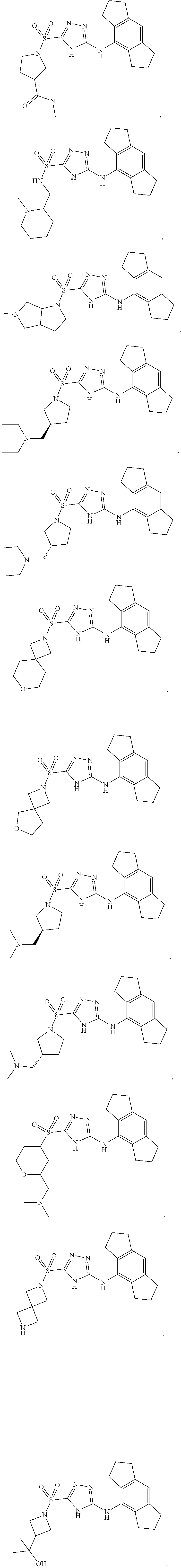

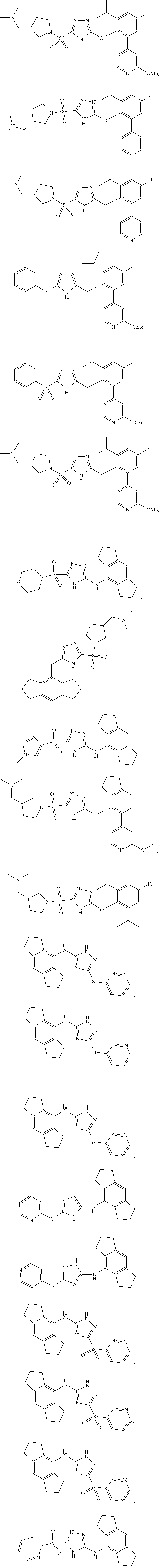

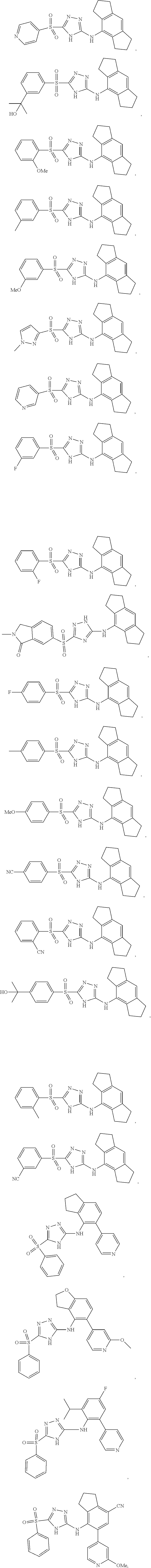

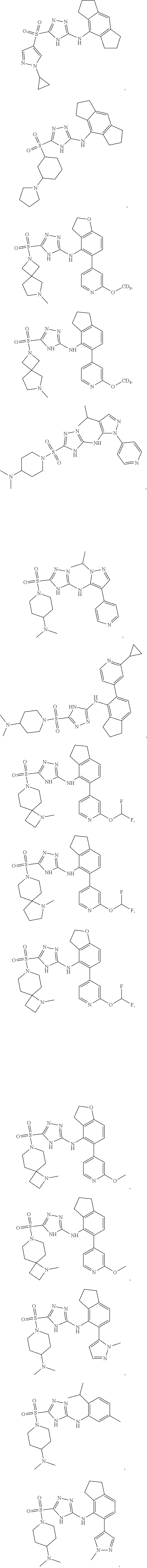

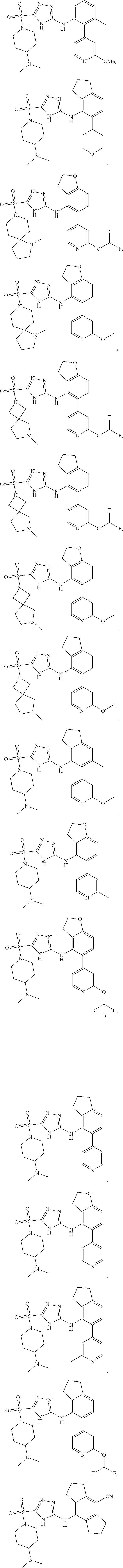

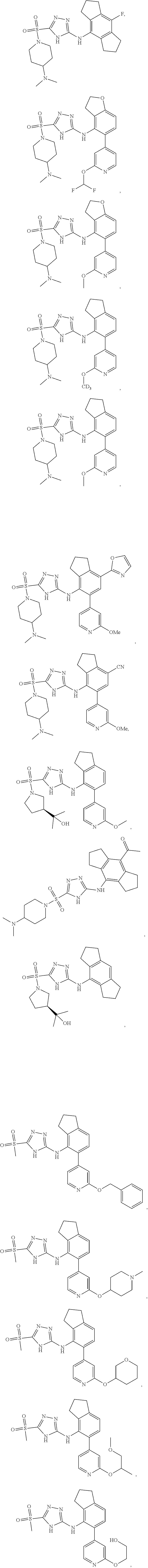

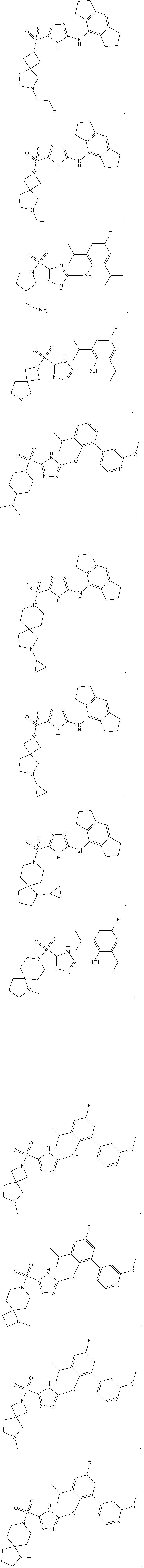

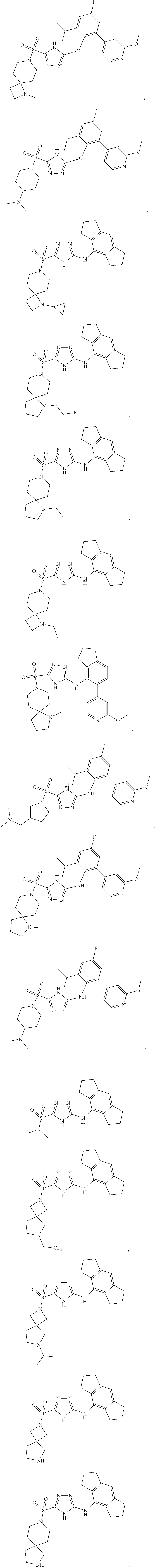

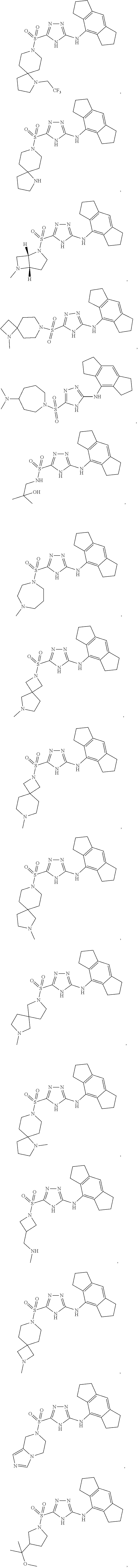

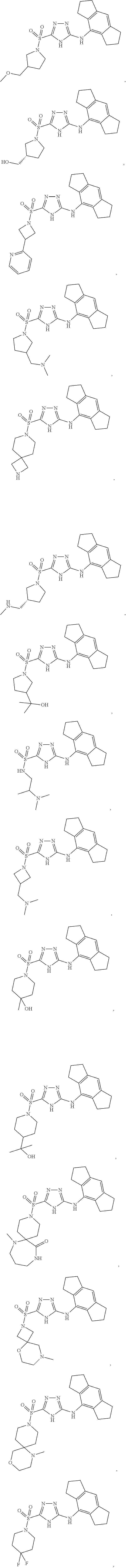

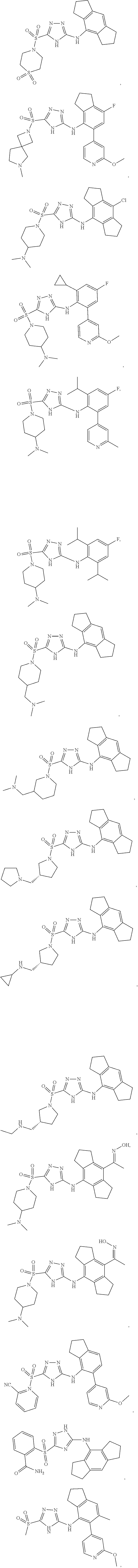

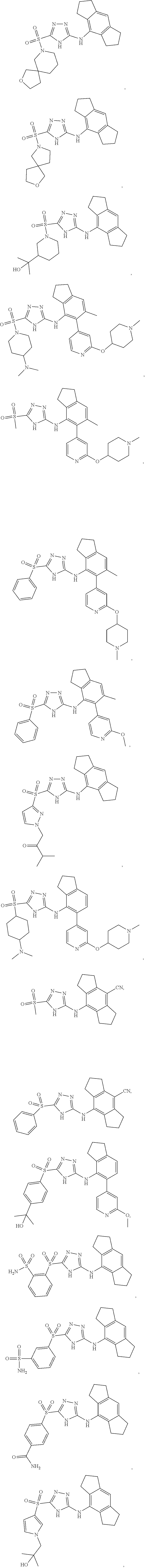

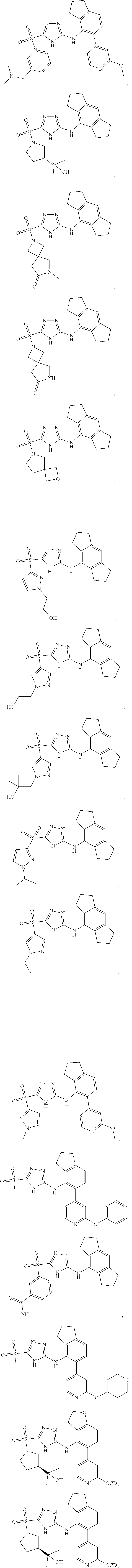

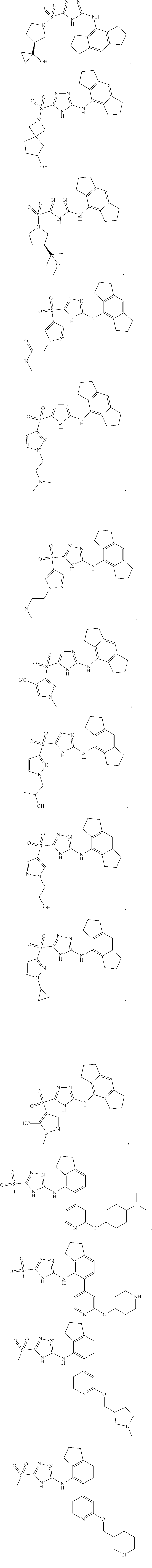

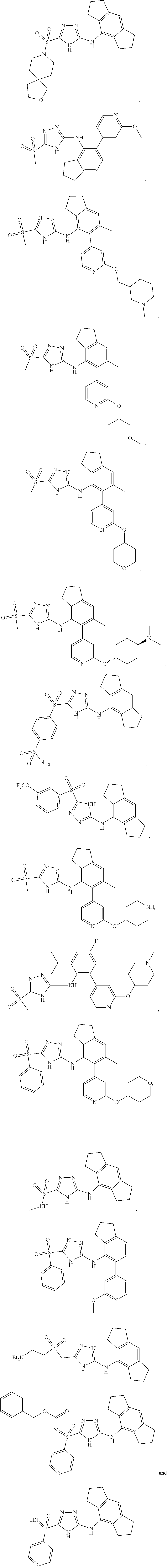

A third aspect of the invention provides a pharmaceutically acceptable salt, solvate or prodrug of any compound of the first or second aspect of the invention.

The compounds of the present invention can be used both in their free base form and their acid addition salt form. For the purposes of this invention, a “salt” of a compound of the present invention includes an acid addition salt. Acid addition salts are preferably pharmaceutically acceptable, non-toxic addition salts with suitable acids, including but not limited to inorganic acids such as hydrohalogenic acids (for example, hydrofluoric, hydrochloric, hydrobromic or hydroiodic acid) or other inorganic acids (for example, nitric, perchloric, sulfuric or phosphoric acid); or organic acids such as organic carboxylic acids (for example, propionic, butyric, glycolic, lactic, mandelic, citric, acetic, benzoic, salicylic, succinic, malic or hydroxysuccinic, tartaric, fumaric, maleic, hydroxymaleic, mucic or galactaric, gluconic, pantothenic or pamoic acid), organic sulfonic acids (for example, methanesulfonic, trifluoromethanesulfonic, ethanesulfonic, 2-hydroxyethanesulfonic, benzenesulfonic, toluene-p-sulfonic, naphthalene-2-sulfonic or camphorsulfonic acid) or amino acids (for example, ornithinic, glutamic or aspartic acid). The acid addition salt may be a mono-, di-, tri- or multi-acid addition salt. A preferred salt is a hydrohalogenic, sulfuric, phosphoric or organic acid addition salt. A preferred salt is a hydrochloric acid addition salt.

Where a compound of the invention includes a quaternary ammonium group, typically the compound is used in its salt form. The counter ion to the quaternary ammonium group may be any pharmaceutically acceptable, non-toxic counter ion. Examples of suitable counter ions include the conjugate bases of the protic acids discussed above in relation to acid-addition salts.

The compounds of the present invention can also be used both, in their free acid form and their salt form. For the purposes of this invention, a “salt” of a compound of the present invention includes one formed between a protic acid functionality (such as a carboxylic acid group) of a compound of the present invention and a suitable cation. Suitable cations include, but are not limited to lithium, sodium, potassium, magnesium, calcium and ammonium. The salt may be a mono-, di-, tri- or multi-salt. Preferably the salt is a mono- or di-lithium, sodium, potassium, magnesium, calcium or ammonium salt. More preferably the salt is a mono- or di-sodium salt or a mono- or di-potassium salt.

Preferably any salt is a pharmaceutically acceptable non-toxic salt. However, in addition to pharmaceutically acceptable salts, other salts are included in the present invention, since they have potential to serve as intermediates in the purification or preparation of other, for example, pharmaceutically acceptable salts, or are useful for identification, characterisation or purification of the free acid or base.

The compounds and/or salts of the present invention may be anhydrous or in the form of a hydrate (e.g. a hemihydrate, monohydrate, dihydrate or trihydrate) or other solvate. Such other solvates may be formed with common organic solvents, including but not limited to, alcoholic solvents e.g. methanol, ethanol or isopropanol.

In some embodiments of the present invention, therapeutically inactive prodrugs are provided. Prodrugs are compounds which, when administered to a subject such as a human, are converted in whole or in part to a compound of the invention. In most embodiments, the prodrugs are pharmacologically inert chemical derivatives that can be converted in vivo to the active drug molecules to exert a therapeutic effect. Any of the compounds described herein can be administered as a prodrug to increase the activity, bioavailability, or stability of the compound or to otherwise alter the properties of the compound. Typical examples of prodrugs include compounds that have biologically labile protecting groups on a functional moiety of the active compound. Prodrugs include, but are not limited to, compounds that can be oxidized, reduced, aminated, deaminated, hydroxylated, dehydroxylated, hydrolyzed, dehydrolyzed, alkylated, dealkylated, acylated, deacylated, phosphorylated, and/or dephosphorylated to produce the active compound. The present invention also encompasses salts and solvates of such prodrugs as described above.

The compounds, salts, solvates and prodrugs of the present invention may contain at least one chiral centre. The compounds, salts, solvates and prodrugs may therefore exist in at least two isomeric forms. The present invention encompasses racemic mixtures of the compounds, salts, solvates and prodrugs of the present invention as well as enantiomerically enriched and substantially enantiomerically pure isomers. For the purposes of this invention, a “substantially enantiomerically pure” isomer of a compound comprises less than 5% of other isomers of the same compound, more typically less than 2%, and most typically less than 0.5% by weight.

The compounds, salts, solvates and prodrugs of the present invention may contain any stable isotope including, but not limited to ¹²C, ¹³C, ¹H, ²H (D), ¹⁴N, ¹⁵N, ¹⁶O, ¹⁷O, ¹⁸O, ¹⁹F and ¹²⁷I, and any radioisotope including, but not limited to ¹C, ¹⁴C, ³H (T), ¹³N, ¹⁵O, ¹⁸F, ¹²³I, ¹²⁴I, ¹²⁵I and ¹³¹I.

The compounds, salts, solvates and prodrugs of the present invention may be in any polymorphic or amorphous form.

A fourth aspect of the invention provides a pharmaceutical composition comprising a compound of the first or second aspect of the invention, or a pharmaceutically acceptable salt, solvate or prodrug of the third aspect of the invention, and a pharmaceutically acceptable excipient.

Conventional procedures for the selection and preparation of suitable pharmaceutical formulations are described in, for example, “Aulton's Pharmaceutics—The Design and Manufacture of Medicines”, M. E. Aulton and K. M. G. Taylor, Churchill Livingstone Elsevier, 4^{th} Ed., 2013.

Pharmaceutically acceptable excipients including adjuvants, diluents or carriers that may be used in the pharmaceutical compositions of the invention are those conventionally employed in the field of pharmaceutical formulation, and include, but are not limited to, sugars, sugar alcohols, starches, ion exchangers, alumina, aluminium stearate, lecithin, serum proteins such as human serum albumin, buffer substances such as phosphates, glycerine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinylpyrrolidone, cellulose-based substances, polyethylene glycol, sodium carboxymethylcellulose, polyacrylates, waxes, polyethylene-polyoxypropylene-block polymers, polyethylene glycol and wool fat.

In one embodiment, the pharmaceutical composition of the fourth aspect of the invention comprises a compound of the first specific embodiment or of the second specific embodiment of the first aspect of the invention, or a pharmaceutically acceptable salt, solvate or prodrug of such a compound.

In one embodiment, the pharmaceutical composition of the fourth aspect of the invention additionally comprises one or more further active agents.

In a further embodiment, the pharmaceutical composition of the fourth aspect of the invention may be provided as a part of a kit of parts, wherein the kit of parts comprises the pharmaceutical composition of the fourth aspect of the invention and one or more further pharmaceutical compositions, wherein the one or more further pharmaceutical compositions each comprise a pharmaceutically acceptable excipient and one or more further active agents.

A fifth aspect of the invention provides a compound of the first or second aspect of the invention, or a pharmaceutically acceptable salt, solvate or prodrug of the third aspect of the invention, or a pharmaceutical composition of the fourth aspect of the invention, for use in medicine, and/or for use in the treatment or prevention of a disease, disorder or condition. Typically, the use comprises the administration of the compound, salt, solvate, prodrug or pharmaceutical composition to a subject. In one embodiment, the use comprises the co-administration of one or more further active agents.

In one embodiment of the fifth aspect of the invention, the compound is a compound of the first specific embodiment or of the second specific embodiment of the first aspect of the invention.

The term “treatment” as used herein refers equally to curative therapy, and ameliorating or palliative therapy. The term includes obtaining beneficial or desired physiological results, which may or may not be established clinically. Beneficial or desired clinical results include, but are not limited to, the alleviation of symptoms, the prevention of symptoms, the diminishment of extent of disease, the stabilisation (i.e., not worsening) of a condition, the delay or slowing of progression/worsening of a condition/symptoms, the amelioration or palliation of the condition/symptoms, and remission (whether partial or total), whether detectable or undetectable. The term “palliation”, and variations thereof, as used herein, means that the extent and/or undesirable manifestations of a physiological condition or symptom are lessened and/or time course of the progression is slowed or lengthened, as compared to not administering a compound, salt, solvate, prodrug or pharmaceutical composition of the present invention. The term “prevention” as used herein in relation to a disease, disorder or condition, relates to prophylactic or preventative therapy, as well as therapy to reduce the risk of developing the disease, disorder or condition. The term “prevention” includes both the avoidance of occurrence of the disease, disorder or condition, and the delay in onset of the disease, disorder or condition. Any statistically significant (p≤0.05) avoidance of occurrence, delay in onset or reduction in risk as measured by a controlled clinical trial may be deemed a prevention of the disease, disorder or condition. Subjects amenable to prevention include those at heightened risk of a disease, disorder or condition as identified by genetic or biochemical markers. Typically, the genetic or biochemical markers are appropriate to the disease, disorder or condition under consideration and may include for example, inflammatory biomarkers such as C-reactive protein (CRP) and monocyte chemoattractant protein 1 (MCP-1) in the case of inflammation; total cholesterol, triglycerides, insulin resistance and C-peptide in the case of NAFLD and NASH; and more generally IL1β and IL18 in the case of a disease, disorder or condition responsive to NLRP3 inhibition.

A sixth aspect of the invention provides the use of a compound of the first or second aspect, or a pharmaceutically effective salt, solvate or prodrug of the third aspect, in the manufacture of a medicament for the treatment or prevention of a disease, disorder or condition. Typically, the treatment or prevention comprises the administration of the compound, salt, solvate, prodrug or medicament to a subject. In one embodiment, the treatment or prevention comprises the co-administration of one or more further active agents.

A seventh aspect of the invention provides a method of treatment or prevention of a disease, disorder or condition, the method comprising the step of administering an effective amount of a compound of the first or second aspect, or a pharmaceutically acceptable salt, solvate or prodrug of the third aspect, or a pharmaceutical composition of the fourth aspect, to thereby treat or prevent the disease, disorder or condition. In one embodiment, the method further comprises the step of co-administering an effective amount of one or more further active agents. Typically, the administration is to a subject in need thereof.

An eighth aspect of the invention provides a compound of the first or second aspect of the invention, or a pharmaceutically acceptable salt, solvate or prodrug of the third aspect of the invention, or a pharmaceutical composition of the fourth aspect of the invention, for use in the treatment or prevention of a disease, disorder or condition in an individual, wherein the individual has a germline or somatic non-silent mutation in NLRP3. The mutation may be, for example, a gain-of-function or other mutation resulting in increased NLRP3 activity. Typically, the use comprises the administration of the compound, salt, solvate, prodrug or pharmaceutical composition to the individual. In one embodiment, the use comprises the co-administration of one or more further active agents. The use may also comprise the diagnosis of an individual having a germline or somatic non-silent mutation in NLRP3, wherein the compound, salt, solvate, prodrug or pharmaceutical composition is administered to an individual on the basis of a positive diagnosis for the mutation. Typically, identification of the mutation in NLRP3 in the individual may be by any suitable genetic or biochemical means.

A ninth aspect of the invention provides the use of a compound of the first or second aspect, or a pharmaceutically effective salt, solvate or prodrug of the third aspect, in the manufacture of a medicament for the treatment or prevention of a disease, disorder or condition in an individual, wherein the individual has a germline or somatic non-silent mutation in NLRP3. The mutation may be, for example, a gain-of-function or other mutation resulting in increased NLRP3 activity. Typically, the treatment or prevention comprises the administration of the compound, salt, solvate, prodrug or medicament to the individual. In one embodiment, the treatment or prevention comprises the co-administration of one or more further active agents. The treatment or prevention may also comprise the diagnosis of an individual having a germline or somatic non-silent mutation in NLRP3, wherein the compound, salt, solvate, prodrug or medicament is administered to an individual on the basis of a positive diagnosis for the mutation. Typically, identification of the mutation in NLRP3 in the individual may be by any suitable genetic or biochemical means.

A tenth aspect of the invention provides a method of treatment or prevention of a disease, disorder or condition, the method comprising the steps of diagnosing of an individual having a germline or somatic non-silent mutation in NLRP3, and administering an effective amount of a compound of the first or second aspect, or a pharmaceutically acceptable salt, solvate or prodrug of the third aspect, or a pharmaceutical composition of the fourth aspect, to the positively diagnosed individual, to thereby treat or prevent the disease, disorder or condition. In one embodiment, the method further comprises the step of co-administering an effective amount of one or more further active agents. Typically, the administration is to a subject in need thereof.

In general embodiments, the disease, disorder or condition may be a disease, disorder or condition of the immune system, the cardiovascular system, the endocrine system, the gastrointestinal tract, the renal system, the hepatic system, the metabolic system, the respiratory system, the central nervous system, may be a cancer or other malignancy, and/or may be caused by or associated with a pathogen.

It will be appreciated that these general embodiments defined according to broad categories of diseases, disorders and conditions are not mutually exclusive. In this regard any particular disease, disorder or condition may be categorized according to more than one of the above general embodiments. A non-limiting example is type I diabetes which is an autoimmune disease and a disease of the endocrine system.

In one embodiment of the fifth, sixth, seventh, eighth, ninth or tenth aspect of the invention, the disease, disorder or condition is responsive to NLRP3 inhibition. As used herein, the term “NLRP3 inhibition” refers to the complete or partial reduction in the level of activity of NLRP3 and includes, for example, the inhibition of active NLRP3 and/or the inhibition of activation of NLRP3.

There is evidence for a role of NLRP3-induced IL-1 and IL-18 in the inflammatory responses occurring in connection with, or as a result of, a multitude of different disorders (Menu et al., Clinical and Experimental Immunology, 166: 1-15, 2011; Strowig et al., Nature, 481:278-286, 2012).

NLRP3 has been implicated in a number of autoinflammatory diseases, including Familial Mediterranean fever (FMF), TNF receptor associated periodic syndrome (TRAPS), hyperimmunoglobulinemia D and periodic fever syndrome (HIDS), pyogenic arthritis, pyoderma gangrenosum and acne (PAPA), Sweet's syndrome, chronic nonbacterial osteomyelitis (CNO), and acne vulgaris (Cook et al., Eur. J. Immunol., 40: 595-653, 2010). In particular, NLRP3 mutations have been found to be responsible for a set of rare autoinflammatory diseases known as CAPS (Ozaki et al., J. Inflammation Research, 8:15-27, 2015; Schroder et al., Cell, 140: 821-832, 2010; and Menu et al., Clinical and Experimental Immunology, 166: 1-15, 2011). CAPS are heritable diseases characterized by recurrent fever and inflammation and are comprised of three autoinflammatory disorders that form a clinical continuum. These diseases, in order of increasing severity, are familial cold autoinflammatory syndrome (FCAS), Muckle-Wells syndrome (MWS), and chronic infantile cutaneous neurological articular syndrome (CINCA; also called neonatal-onset multisystem inflammatory disease, NOMID), and all have been shown to result from gain-of-function mutations in the NLRP3 gene, which leads to increased secretion of IL-1β.

A number of autoimmune diseases have been shown to involve NLRP3 including, in particular, multiple sclerosis, type-1 diabetes (T1D), psoriasis, rheumatoid arthritis (RA), Behcet's disease, Schnitzler syndrome, macrophage activation syndrome (Masters Clin. Immunol. 2013; Braddock et al. Nat. Rev. Drug Disc. 2004 3: 1-10; Inoue et al., Immunology 139: 11-18, Coll et al. Nat. Med. 2015 21(3):248-55; and Scott et al. Clin. Exp. Rheumatol 2016 34(1): 88-93), systemic lupus erythematosus (Lu et al. J Immunol. 2017198(3): 1119-29), and systemic sclerosis (Artlett et al. Arthritis Rheum. 2011; 63(11): 3563-74). NLRP3 has also been shown to play a role in a number of lung diseases including chronic obstructive pulmonary disorder (COPD), asthma (including steroid-resistant asthma), asbestosis, and silicosis (De Nardo et al., Am. J. Pathol.,184: 42-54, 2014 and Kim et al. Am J Respir Crit Care Med. 2017196(3): 283-97). NLRP3 has also been suggested to have a role in a number of central nervous system conditions, including Parkinson's disease (PD), Alzheimer's disease (AD), dementia, Huntington's disease, cerebral malaria, brain injury from pneumococcal meningitis (Walsh et al., Nature Reviews, 15: 84-97, 2014, and Dempsey et al. Brain. Behav. Immun. 2017 61: 306-316), intracranial aneurysms (Zhang et al. J. Stroke & Cerebrovascular Dis. 2015 24; 5: 972-979), and traumatic brain injury (Ismael et al. J Neurotrauma. 2018 Jan. 2). NRLP3 activity has also been shown to be involved in various metabolic diseases including type 2 diabetes (T2D), atherosclerosis, obesity, gout, pseudo-gout, metabolic syndrome (Wen et al., Nature Immunology, 13: 352-357, 2012; Duewell et al., Nature, 464: 1357-1361, 2010; Strowig et al., Nature, 481: 278-286, 2012), and non-alcoholic steatohepatitis (Mridha et al. J Hepatol. 2017 66(5): 1037-46). A role for NLRP3 via IL-1p has also been suggested in atherosclerosis, myocardial infarction (van Hout et al. Eur. Heart J. 2017 38(11): 828-36), heart failure (Sano et al. J AM. Coll. Cardiol. 2018 71(8): 875-66), aortic aneurysm and dissection (Wu et al. Arterioscler. Thromb. Vasc. Biol. 2017 37(4): 694-706), and other cardiovascular events (Ridker et al., N Engl J Med., doi: 10.1056/NEJMoa1707914, 2017). Other diseases in which NLRP3 has been shown to be involved include: ocular diseases such as both wet and dry age-related macular degeneration (Doyle et al., Nature Medicine, 18: 791-798, 2012 and Tarallo et al. Cell 2012149(4): 847-59), diabetic retinopathy (Loukovaara et al. Acta Ophthalmol. 2017; 95(8): 803-808) and optic nerve damage (Puyang et al. Sci Rep. 2016 Feb. 19; 6:20998); liver diseases including non-alcoholic steatohepatitis (NASH) (Henao-Meija et al., Nature, 482: 179-185, 2012); inflammatory reactions in the lung and skin (Primiano et al. J Immunol. 2016 197(6): 2421-33) including contact hypersensitivity (such as bullous pemphigoid (Fang et al. J Dermatol Sci. 2016; 83(2): 116-23)), atopic dermatitis (Niebuhr et al. Allergy 2014 69(8): 1058-67), Hidradenitis suppurativa (Alikhan et al. 2009 J Am Acad Dermatol 60(4): 539-61), acne vulgaris (Qin et al. J Invest. Dermatol. 2014 134(2): 381-88), and sarcoidosis (Jager et al. Am J Respir Crit Care Med 2015 191: A5816); inflammatory reactions in the joints (Braddock et al., Nat. Rev. Drug Disc., 3: 1-10, 2004); amyotrophic lateral sclerosis (Gugliandolo et al. Inflammation 2018 41(1): 93-103); cystic fibrosis (Iannitti et al. Nat. Commun. 2016 7: 10791); stroke (Walsh et al., Nature Reviews, 15: 84-97, 2014); chronic kidney disease (Granata et al. PLoS One 2015 10(3): eo122272); and inflammatory bowel diseases including ulcerative colitis and Crohn's disease (Braddock et al., Nat. Rev. Drug Disc., 3: 1-10, 2004, Neudecker et al. J Exp. Med. 2017 214(6): 1737-52, and Lazaridis et al. Dig. Dis. Sci. 2017 62(9): 2348-56). The NLRP3 inflammasome has been found to be activated in response to oxidative stress, and UVB irradiation (Schroder et al., Science, 327: 296-300, 2010). NLRP3 has also been shown to be involved in inflammatory hyperalgesia (Dolunay et al., Inflammation, 40: 366-386, 2017).

The inflammasome, and NLRP3 specifically, has also been proposed as a target for modulation by various pathogens including viruses such as DNA viruses (Amsler et al., Future Virol. (2013) 8(4), 357-370).

NLRP3 has also been implicated in the pathogenesis of many cancers (Menu et al., Clinical and Experimental Immunology 166: 1-15, 2011; and Masters Clin. Immunol. 2013). For example, several previous studies have suggested a role for IL-1p in cancer invasiveness, growth and metastasis, and inhibition of IL-1p with canakinumab has been shown to reduce the incidence of lung cancer and total cancer mortality in a randomised, double-blind, placebo-controlled trial (Ridker et al. Lancet, S0140-6736 (17)₃₂₂₄₇-X, 2017). Inhibition of the NLRP3 inflammasome or IL-1p has also been shown to inhibit the proliferation and migration of lung cancer cells in vitro (Wang et al. Oncol Rep. 2016; 35(4): 2053-64). A role for the NLRP3 inflammasome has been suggested in myelodysplastic syndromes (Basiorka et al. Blood. 2016 Dec. 22; 128(25):2960-2975) and also in the carcinogenesis of various other cancers including glioma (Li et al. Am J Cancer Res. 2015; 5(1): 442-449), inflammation-induced tumours (Allen et al. J Exp Med. 2010; 207(5): 1045-56 and Hu et al. PNAS. 2010; 107(50): 21635-40), multiple myeloma (Li et al. Hematology 2016 21(3): 144-51), and squamous cell carcinoma of the head and neck (Huang et al. J Exp Clin Cancer Res. 2017 2; 36(1): 116). Activation of the NLRP3 inflammasome has also been shown to mediate chemoresistance of tumour cells to 5-Fluorouracil (Feng et al. J Exp Clin Cancer Res. 2017 21; 36(1): 81), and activation of NLRP3 inflammasome in peripheral nerve contributes to chemotherapy-induced neuropathic pain (Jia et al. Mol Pain. 2017; 13:1-11).

NLRP3 has also been shown to be required for the efficient control of viral, bacterial, fungal, and helminth pathogen infections (Strowig et al., Nature, 481:278-286, 2012).

Accordingly, examples of diseases, disorders or conditions which may be responsive to NLRP3 inhibition and which may be treated or prevented in accordance with the fifth, sixth, seventh, eighth, ninth or tenth aspect of the present invention include:

(i) inflammation, including inflammation occurring as a result of an inflammatory disorder, e.g. an autoinflammatory disease, inflammation occurring as a symptom of a non-inflammatory disorder, inflammation occurring as a result of infection, or inflammation secondary to trauma, injury or autoimmunity;

(ii) auto-immune diseases such as acute disseminated encephalitis, Addison's disease, ankylosing spondylitis, antiphospholipid antibody syndrome (APS), anti-synthetase syndrome, aplastic anemia, autoimmune adrenalitis, autoimmune hepatitis, autoimmune oophoritis, autoimmune polyglandular failure, autoimmune thyroiditis, Coeliac disease, Crohn's disease, type 1 diabetes (T1D), Goodpasture's syndrome, Graves' disease, Guillain-Barré syndrome (GBS), Hashimoto's disease, idiopathic thrombocytopenic purpura, Kawasaki's disease, lupus erythematosus including systemic lupus erythematosus (SLE), multiple sclerosis (MS) including primary progressive multiple sclerosis (PPMS), secondary progressive multiple sclerosis (SPMS) and relapsing remitting multiple sclerosis (RRMS), myasthenia gravis, opsoclonus myoclonus syndrome (OMS), optic neuritis, Ord's thyroiditis, pemphigus, pernicious anaemia, polyarthritis, primary biliary cirrhosis, rheumatoid arthritis (RA), psoriatic arthritis, juvenile idiopathic arthritis or Still's disease, refractory gouty arthritis, Reiter's syndrome, Sjögren's syndrome, systemic sclerosis a systemic connective tissue disorder, Takayasu's arteritis, temporal arteritis, warm autoimmune hemolytic anemia, Wegener's granulomatosis, alopecia universalis, Behget's disease, Chagas' disease, dysautonomia, endometriosis, hidradenitis suppurativa (HS), interstitial cystitis, neuromyotonia, psoriasis, sarcoidosis, scleroderma, ulcerative colitis, Schnitzler syndrome, macrophage activation syndrome, Blau syndrome, vitiligo or vulvodynia; (iii) cancer including lung cancer, pancreatic cancer, gastric cancer, myelodysplastic syndrome, leukaemia including acute lymphocytic leukaemia (ALL) and acute myeloid leukaemia (AML), adrenal cancer, anal cancer, basal and squamous cell skin cancer, bile duct cancer, bladder cancer, bone cancer, brain and spinal cord tumours, breast cancer, cervical cancer, chronic lymphocytic leukaemia (CLL), chronic myeloid leukaemia (CML), chronic myelomonocytic leukaemia (CMML), colorectal cancer, endometrial cancer, oesophagus cancer, Ewing family of tumours, eye cancer, gallbladder cancer, gastrointestinal carcinoid tumours, gastrointestinal stromal tumour (GIST), gestational trophoblastic disease, glioma, Hodgkin lymphoma, Kaposi sarcoma, kidney cancer, laryngeal and hypopharyngeal cancer, liver cancer, lung carcinoid tumour, lymphoma including cutaneous T cell lymphoma, malignant mesothelioma, melanoma skin cancer, Merkel cell skin cancer, multiple myeloma, nasal cavity and paranasal sinuses cancer, nasopharyngeal cancer, neuroblastoma, non-Hodgkin lymphoma, non-small cell lung cancer, oral cavity and oropharyngeal cancer, osteosarcoma, ovarian cancer, penile cancer, pituitary tumours, prostate cancer, retinoblastoma, rhabdomyosarcoma, salivary gland cancer, skin cancer, small cell lung cancer, small intestine cancer, soft tissue sarcoma, stomach cancer, testicular cancer, thymus cancer, thyroid cancer including anaplastic thyroid cancer, uterine sarcoma, vaginal cancer, vulvar cancer, Waldenstrom macroglobulinemia, and Wilms tumour; (iv) infections including viral infections (e.g. from influenza virus, human immunodeficiency virus (HIV), alphavirus (such as Chikungunya and Ross River virus), flaviviruses (such as Dengue virus and Zika virus), herpes viruses (such as Epstein Barr Virus, cytomegalovirus, Varicella-zoster virus, and KSHV), poxviruses (such as vaccinia virus (Modified vaccinia virus Ankara) and Myxoma virus), adenoviruses (such as Adenovirus 5), or papillomavirus), bacterial infections (e.g. from Staphylococcus aureus, Helicobacter pylori, Bacillus anthracis, Bordatella pertussis, Burkholderia pseudomallei, Corynebacterium diptheriae, Clostridium tetani, Clostridium botulinum, Streptococcus pneumoniae, Streptococcus pyogenes, Listeria monocytogenes, Hemophilus influenzae, Pasteurella multicida, Shigella dysenteriae, Mycobacterium tuberculosis, Mycobacterium leprae, Mycoplasma pneumoniae, Mycoplasma hominis, Neisseria meningitidis, Neisseria gonorrhoeae, Rickettsia rickettsii, Legionella pneumophila, Klebsiella pneumoniae, Pseudomonas aeruginosa, Propionibacterium acnes, Treponema pallidum, Chlamydia trachomatis, Vibrio cholerae, Salmonella typhimurium, Salmonella typhi, Borrelia burgdorferi or Yersinia pestis), fungal infections (e.g. from Candida or Aspergillus species), protozoan infections (e.g. from Plasmodium, Babesia, Giardia, Entamoeba, Leishmania or Trypanosomes), helminth infections (e.g. from schistosoma, roundworms, tapeworms or flukes) and prion infections;

(v) central nervous system diseases such as Parkinson's disease, Alzheimer's disease, dementia, motor neuron disease, Huntington's disease, cerebral malaria, brain injury from pneumococcal meningitis, intracranial aneurysms, traumatic brain injury, and amyotrophic lateral sclerosis;

(vi) metabolic diseases such as type 2 diabetes (T2D), atherosclerosis, obesity, gout, and pseudo-gout;

(vii) cardiovascular diseases such as hypertension, ischaemia, reperfusion injury including post-MI ischemic reperfusion injury, stroke including ischemic stroke, transient ischemic attack, myocardial infarction including recurrent myocardial infarction, heart failure including congestive heart failure and heart failure with preserved ejection fraction, embolism, aneurysms including abdominal aortic aneurysm, and pericarditis including Dressler's syndrome;

(viii) respiratory diseases including chronic obstructive pulmonary disorder (COPD), asthma such as allergic asthma and steroid-resistant asthma, asbestosis, silicosis, nanoparticle induced inflammation, cystic fibrosis and idiopathic pulmonary fibrosis;

(ix) liver diseases including non-alcoholic fatty liver disease (NAFLD) and non-alcoholic steatohepatitis (NASH) including advanced fibrosis stages F3 and F4; alcoholic fatty liver disease (AFLD), and alcoholic steatohepatitis (ASH);

(x) renal diseases including chronic kidney disease, oxalate nephropathy, nephrocalcinosis, glomerulonephritis, and diabetic nephropathy;

(xi) ocular diseases including those of the ocular epithelium, age-related macular degeneration (AMD) (dry and wet), uveitis, corneal infection, diabetic retinopathy, optic nerve damage, dry eye, and glaucoma;

(xii) skin diseases including dermatitis such as contact dermatitis and atopic dermatitis, contact hypersensitivity, sunburn, skin lesions, hidradenitis suppurativa (HS), other cyst-causing skin diseases, and acne conglobata;

(xiii) lymphatic conditions such as lymphangitis and Castleman's disease;

(xiv) psychological disorders such as depression and psychological stress;

(xv) graft versus host disease;

(xvi) allodynia including mechanical allodynia; and

(xvii) any disease where an individual has been determined to carry a germline or somatic non-silent mutation in NLRP3.

In one embodiment, the disease, disorder or condition is selected from:

(i) inflammation;

(ii) an auto-immune disease;

(iii) cancer;

(iv) an infection;

(v) a central nervous system disease;

(vi) a metabolic disease;

(vii) a cardiovascular disease;

(viii) a respiratory disease;

(ix) a liver disease;

(x) a renal disease;

(xi) an ocular disease;

(xii) a skin disease;

(xiii) a lymphatic condition;

(xiv) a psychological disorder;

(xv) graft versus host disease; and

(xvi) any disease where an individual has been determined to carry a germline or somatic non-silent mutation in NLRP3.

In one embodiment, the disease, disorder or condition is selected from:

(i) cancer;

(ii) an infection;

(iii) a central nervous system disease;

(iv) a cardiovascular disease;

(v) a liver disease;

(vi) an ocular disease; or

(vii) a skin disease.

More typically, the disease, disorder or condition is selected from:

(i) cancer;

(ii) an infection;

(iii) a central nervous system disease; or

(iv) a cardiovascular disease.

In one embodiment, the disease, disorder or condition is selected from:

(i) acne conglobata;

(ii) atopic dermatitis;

(iii) Alzheimer's disease;

(iv) amyotrophic lateral sclerosis;

(v) age-related macular degeneration (AMD);

(vi) anaplastic thyroid cancer;

(vii) cryopyrin-associated periodic syndromes (CAPS);

(viii) contact dermatitis;

(ix) cystic fibrosis;

(x) congestive heart failure;

(xi) chronic kidney disease;

(xii) Crohn's disease;

(xiii) familial cold autoinflammatory syndrome (FCAS);

(xiv) Huntington's disease;

(xv) heart failure;

(xvi) heart failure with preserved ejection fraction;

(xvii) ischemic reperfusion injury;

(xviii) juvenile idiopathic arthritis;

(xix) myocardial infarction;

(xx) macrophage activation syndrome;

(xxi) myelodysplastic syndrome;

(xxii) multiple myeloma;

(xxiii) motor neuron disease;

(xxiv) multiple sclerosis;

(xxv) Muckle-Wells syndrome;

(xxvi) non-alcoholic steatohepatitis (NASH);

(xxvii) neonatal-onset multisystem inflammatory disease (NOMID);

(xxviii) Parkinson's disease;

(xxix) systemic juvenile idiopathic arthritis;

(xxx) systemic lupus erythematosus;

(xxxi) traumatic brain injury;

(xxxii) transient ischemic attack; and

(xxxiii) ulcerative colitis.

In a further typical embodiment of the invention, the disease, disorder or condition is inflammation. Examples of inflammation that may be treated or prevented in accordance with the fifth, sixth, seventh, eighth, ninth or tenth aspect of the present invention include inflammatory responses occurring in connection with, or as a result of:

(i) a skin condition such as contact hypersensitivity, bullous pemphigoid, sunburn, psoriasis, atopical dermatitis, contact dermatitis, allergic contact dermatitis, seborrhoetic dermatitis, lichen planus, scleroderma, pemphigus, epidermolysis bullosa, urticaria, erythemas, or alopecia;

(ii) a joint condition such as osteoarthritis, systemic juvenile idiopathic arthritis, adult-onset Still's disease, relapsing polychondritis, rheumatoid arthritis, juvenile chronic arthritis, gout, or a seronegative spondyloarthropathy (e.g. ankylosing spondylitis, psoriatic arthritis or Reiter's disease);

(iii) a muscular condition such as polymyositis or myasthenia gravis; (iv) a gastrointestinal tract condition such as inflammatory bowel disease (including Crohn's disease and ulcerative colitis), gastric ulcer, coeliac disease, proctitis, pancreatitis, eosinopilic gastro-enteritis, mastocytosis, antiphospholipid syndrome, or a food-related allergy which may have effects remote from the gut (e.g., migraine, rhinitis or eczema);

(v) a respiratory system condition such as chronic obstructive pulmonary disease (COPD), asthma (including bronchial, allergic, intrinsic, extrinsic or dust asthma, and particularly chronic or inveterate asthma, such as late asthma and airways hyper-responsiveness), bronchitis, rhinitis (including acute rhinitis, allergic rhinitis, atrophic rhinitis, chronic rhinitis, rhinitis caseosa, hypertrophic rhinitis, rhinitis pumlenta, rhinitis sicca, rhinitis medicamentosa, membranous rhinitis, seasonal rhinitis e.g. hay fever, and vasomotor rhinitis), sinusitis, idiopathic pulmonary fibrosis (IPF), sarcoidosis, farmer's lung, silicosis, asbestosis, adult respiratory distress syndrome, hypersensitivity pneumonitis, or idiopathic interstitial pneumonia;

(vi) a vascular condition such as atherosclerosis, Behcet's disease, vasculitides, or wegener's granulomatosis;

(vii) an autoimmune condition such as systemic lupus erythematosus, Sjogren's syndrome, systemic sclerosis, Hashimoto's thyroiditis, type I diabetes, idiopathic thrombocytopenia purpura, or Graves disease;

(viii) an ocular condition such as uveitis, allergic conjunctivitis, or vernal conjunctivitis;

(ix) a nervous condition such as multiple sclerosis or encephalomyelitis;

(x) an infection or infection-related condition, such as Acquired Immunodeficiency Syndrome (AIDS), acute or chronic bacterial infection, acute or chronic parasitic infection, acute or chronic viral infection, acute or chronic fungal infection, meningitis, hepatitis (A, B or C, or other viral hepatitis), peritonitis, pneumonia, epiglottitis, malaria, dengue hemorrhagic fever, leishmaniasis, streptococcal myositis, Mycobacterium tuberculosis, Mycobacterium avium intracellulare, Pneumocystis carinii pneumonia, orchitis/epidydimitis, legionella, Lyme disease, influenza A, epstein-barr virus, viral encephalitis/aseptic meningitis, or pelvic inflammatory disease;

(xi) a renal condition such as mesangial proliferative glomerulonephritis, nephrotic syndrome, nephritis, glomerular nephritis, acute renal failure, uremia, or nephritic syndrome;

(xii) a lymphatic condition such as Castleman's disease;

(xiii) a condition of, or involving, the immune system, such as hyper IgE syndrome, lepromatous leprosy, familial hemophagocytic lymphohistiocytosis, or graft versus host disease;

(xiv) a hepatic condition such as chronic active hepatitis, non-alcoholic steatohepatitis (NASH), alcohol-induced hepatitis, non-alcoholic fatty liver disease (NAFLD), alcoholic fatty liver disease (AFLD), alcoholic steatohepatitis (ASH) or primary biliary cirrhosis;

(xv) a cancer, including those cancers listed above;

(xvi) a burn, wound, trauma, haemorrhage or stroke;

(xvii) radiation exposure; and/or

(xviii) obesity; and/or

(xix) pain such as inflammatory hyperalgesia.

In one embodiment of the fifth, sixth, seventh, eighth, ninth or tenth aspect of the present invention, the disease, disorder or condition is an autoinflammatory disease such as cryopyrin-associated periodic syndromes (CAPS), Muckle-Wells syndrome (MWS), familial cold autoinflammatory syndrome (FCAS), familial Mediterranean fever (FMF), neonatal onset multisystem inflammatory disease (NOMID), Tumour Necrosis Factor (TNF) Receptor-Associated Periodic Syndrome (TRAPS), hyperimmunoglobulinemia D and periodic fever syndrome (HIDS), deficiency of interleukin 1 receptor antagonist (DIRA), Majeed syndrome, pyogenic arthritis, pyoderma gangrenosum and acne syndrome (PAPA), adult-onset Still's disease (AOSD), haploinsufficiency of A20 (HA20), pediatric granulomatous arthritis (PGA), PLCG2-associated antibody deficiency and immune dysregulation (PLAID), PLCG2-associated autoinflammatory, antibody deficiency and immune dysregulation (APLAID), or sideroblastic anaemia with B-cell immunodeficiency, periodic fevers and developmental delay (SIFD).

Examples of diseases, disorders or conditions which may be responsive to NLRP3 inhibition and which may be treated or prevented in accordance with the fifth, sixth, seventh, eighth, ninth or tenth aspect of the present invention are listed above. Some of these diseases, disorders or conditions are substantially or entirely mediated by NLRP3 inflammasome activity, and NLRP3-induced IL-1β and/or IL-18. As a result, such diseases, disorders or conditions may be particularly responsive to NLRP3 inhibition and may be particularly suitable for treatment or prevention in accordance with the fifth, sixth, seventh, eighth, ninth or tenth aspect of the present invention. Examples of such diseases, disorders or conditions include cryopyrin-associated periodic syndromes (CAPS), Muckle-Wells syndrome (MWS), familial cold autoinflammatory syndrome (FCAS), neonatal onset multisystem inflammatory disease (NOMID), familial Mediterranean fever (FMF), pyogenic arthritis, pyoderma gangrenosum and acne syndrome (PAPA), hyperimmunoglobulinemia D and periodic fever syndrome (HIDS), Tumour Necrosis Factor (TNF) Receptor-Associated Periodic Syndrome (TRAPS), systemic juvenile idiopathic arthritis, adult-onset Still's disease (AOSD), relapsing polychondritis, Schnitzler's syndrome, Sweet's syndrome, Behcet's disease, anti-synthetase syndrome, deficiency of interleukin 1 receptor antagonist (DIRA), and haploinsufficiency of A20 (HA20).

Moreover, some of the diseases, disorders or conditions mentioned above arise due to mutations in NLRP3, in particular, resulting in increased NLRP3 activity. As a result, such diseases, disorders or conditions may be particularly responsive to NLRP3 inhibition and may be particularly suitable for treatment or prevention in accordance with the fifth, sixth, seventh, eighth, ninth or tenth aspect of the present invention. Examples of such diseases, disorders or conditions include cryopyrin-associated periodic syndromes (CAPS), Muckle-Wells syndrome (MWS), familial cold autoinflammatory syndrome (FCAS), and neonatal onset multisystem inflammatory disease (NOMID).

An eleventh aspect of the invention provides a method of inhibiting NLRP3, the method comprising the use of a compound of the first or second aspect of the invention, or a pharmaceutically acceptable salt, solvate or prodrug of the third aspect of the invention, or a pharmaceutical composition of the fourth aspect of the invention, to inhibit NLRP3.

In one embodiment of the eleventh aspect of the present invention, the method comprises the use of a compound of the first or second aspect of the invention, or a pharmaceutically acceptable salt, solvate or prodrug of the third aspect of the invention, or a pharmaceutical composition of the fourth aspect of the invention, in combination with one or more further active agents.

In one embodiment of the eleventh aspect of the present invention, the method is performed ex vivo or in vitro, for example in order to analyse the effect on cells of NLRP3 inhibition.

In another embodiment of the eleventh aspect of the present invention, the method is performed in vivo. For example, the method may comprise the step of administering an effective amount of a compound of the first or second aspect, or a pharmaceutically acceptable salt, solvate or prodrug of the third aspect, or a pharmaceutical composition of the fourth aspect, to thereby inhibit NLRP3. In one embodiment, the method further comprises the step of co-administering an effective amount of one or more further active agents. Typically, the administration is to a subject in need thereof.

Alternately, the method of the eleventh aspect of the invention may be a method of inhibiting NLRP3 in a non-human animal subject, the method comprising the steps of administering the compound, salt, solvate, prodrug or pharmaceutical composition to the non-human animal subject and optionally subsequently mutilating or sacrificing the non-human animal subject. Typically, such a method further comprises the step of analysing one or more tissue or fluid samples from the optionally mutilated or sacrificed non-human animal subject. In one embodiment, the method further comprises the step of co-administering an effective amount of one or more further active agents.

A twelfth aspect of the invention provides a compound of the first or second aspect of the invention, or a pharmaceutically acceptable salt, solvate or prodrug of the third aspect of the invention, or a pharmaceutical composition of the fourth aspect of the invention, for use in the inhibition of NLRP3. Typically, the use comprises the administration of the compound, salt, solvate, prodrug or pharmaceutical composition to a subject. In one embodiment, the compound, salt, solvate, prodrug or pharmaceutical composition is co-administered with one or more further active agents.

A thirteenth aspect of the invention provides the use of a compound of the first or second aspect of the invention, or a pharmaceutically effective salt, solvate or prodrug of the third aspect of the invention, in the manufacture of a medicament for the inhibition of NLRP3. Typically, the inhibition comprises the administration of the compound, salt, solvate, prodrug or medicament to a subject. In one embodiment, the compound, salt, solvate, prodrug or medicament is co-administered with one or more further active agents.

In any embodiment of any of the fifth to thirteenth aspects of the present invention that comprises the use or co-administration of one or more further active agents, the one or more further active agents may comprise for example one, two or three different further active agents.

The one or more further active agents may be used or administered prior to, simultaneously with, sequentially with or subsequent to each other and/or to the compound of the first or second aspect of the invention, the pharmaceutically acceptable salt, solvate or prodrug of the third aspect of the invention, or the pharmaceutical composition of the fourth aspect of the invention. Where the one or more further active agents are administered simultaneously with the compound of the first or second aspect of the invention, or the pharmaceutically acceptable salt, solvate or prodrug of the third aspect of the invention, a pharmaceutical composition of the fourth aspect of the invention may be administered wherein the pharmaceutical composition additionally comprises the one or more further active agents.

In one embodiment of any of the fifth to thirteenth aspects of the present invention that comprises the use or co-administration of one or more further active agents, the one or more further active agents are selected from:

(i) chemotherapeutic agents;

(ii) antibodies;

(iii) alkylating agents;

(iv) anti-metabolites;

(v) anti-angiogenic agents;

(vi) plant alkaloids and/or terpenoids;

(vii) topoisomerase inhibitors;

(viii) mTOR inhibitors;

(ix) stilbenoids;

(x) STING agonists;

(xi) cancer vaccines;

(xii) immunomodulatory agents;

(xiii) antibiotics;

(xiv) anti-fungal agents;

(xv) anti-helminthic agents; and/or

(xvi) other active agents.

It will be appreciated that these general embodiments defined according to broad categories of active agents are not mutually exclusive. In this regard any particular active agent may be categorized according to more than one of the above general embodiments. A non-limiting example is urelumab which is an antibody that is an immunomodulatory agent for the treatment of cancer.

In some embodiments, the one or more chemotherapeutic agents are selected from abiraterone acetate, altretamine, amsacrine, anhydrovinblastine, auristatin, azathioprine, adriamycin, bexarotene, bicalutamide, BMS 184476, bleomycin, N,N-dimethyl-L-valyl-L-valyl-N-methyl-L-valyl-L-prolyl-L-proline-t-butylamide, cisplatin, carboplatin, carboplatin cyclophosphamide, chlorambucil, cachectin, cemadotin, cyclophosphamide, carmustine, cryptophycin, cytarabine, docetaxel, doxetaxel, doxorubicin, dacarbazine (DTIC), dactinomycin, daunorubicin, decitabine, dolastatin, etoposide, etoposide phosphate, enzalutamide (MDV3100), 5-fluorouracil, fludarabine, flutamide, gemcitabine, hydroxyurea and hydroxyureataxanes, idarubicin, ifosfamide, irinotecan, leucovorin, lonidamine, lomustine (CCNU), larotaxel (RPR109881), mechlorethamine, mercaptopurine, methotrexate, mitomycin C, mitoxantrone, melphalan, mivobulin, 3′,4′-didehydro-4′-deoxy-8′-norvin-caleukoblastine, nilutamide, oxaliplatin, onapristone, prednimustine, procarbazine, paclitaxel, platinum-containing anti-cancer agents, 2,3,4,5,6-pentafluoro-N-(3-fluoro-4-methoxyphenyl)benzene sulfonamide, prednimustine, procarbazine, rhizoxin, sertenef, streptozocin, stramustine phosphate, tretinoin, tasonermin, taxol, topotecan, tamoxifen, teniposide, taxane, tegafur/uracil, vincristine, vinblastine, vinorelbine, vindesine, vindesine sulfate, and/or vinflunine.

Alternatively or in addition, the one or more chemotherapeutic agents may be selected from CD59 complement fragment, fibronectin fragment, gro-beta (CXCL2), heparinases, heparin hexasaccharide fragment, human chorionic gonadotropin (hCG), interferon alpha, interferon beta, interferon gamma, interferon inducible protein (IP-10), interleukin-12, kringle 5 (plasminogen fragment), metalloproteinase inhibitors (TIMPs), 2-methoxyestradiol, placental ribonuclease inhibitor, plasminogen activator inhibitor, platelet factor-4 (PF4), prolactin 16 kD fragment, proliferin-related protein (PRP), various retinoids, tetrahydrocortisol-S, thrombospondin-1 (TSP-1), transforming growth factor-beta (TGF-β), vasculostatin, vasostatin (calreticulin fragment), and/or cytokines (including interleukins, such as interleukin-2 (IL-2), or IL-10).

In some embodiments, the one or more antibodies may comprise one or more monoclonal antibodies. In some embodiments, the one or more antibodies are selected from abciximab, adalimumab, alemtuzumab, atlizumab, basiliximab, belimumab, bevacizumab, bretuximab vedotin, canakinumab, cetuximab, ceertolizumab pegol, daclizumab, denosumab, eculizumab, efalizumab, gemtuzumab, golimumab, ibritumomab tiuxetan, infliximab, ipilimumab, muromonab-CD3, natalizumab, ofatumumab, omalizumab, palivizumab, panitumuab, ranibizumab, rituximab, tocilizumab, tositumomab, and/or trastuzumab.

In some embodiments, the one or more alkylating agents may comprise an agent capable of alkylating nucleophilic functional groups under conditions present in cells, including, for example, cancer cells. In some embodiments, the one or more alkylating agents are selected from cisplatin, carboplatin, mechlorethamine, cyclophosphamide, chlorambucil, ifosfamide and/or oxaliplatin. In some embodiments, the alkylating agent may function by impairing cell function by forming covalent bonds with amino, carboxyl, sulfhydryl, and/or phosphate groups in biologically important molecules. In some embodiments, the alkylating agent may function by modifying a cell's DNA.

In some embodiments, the one or more anti-metabolites may comprise an agent capable of affecting or preventing RNA or DNA synthesis. In some embodiments, the one or more anti-metabolites are selected from azathioprine and/or mercaptopurine.

In some embodiments, the one or more anti-angiogenic agents are selected from endostatin, angiogenin inhibitors, angiostatin, angioarrestin, angiostatin (plasminogen fragment), basement-membrane collagen-derived anti-angiogenic factors (tumstatin, canstatin, or arrestin), anti-angiogenic antithrombin III, and/or cartilage-derived inhibitor (CDI).

In some embodiments, the one or more plant alkaloids and/or terpenoids may prevent microtubule function. In some embodiments, the one or more plant alkaloids and/or terpenoids are selected from a vinca alkaloid, a podophyllotoxin and/or a taxane. In some embodiments, the one or more vinca alkaloids may be derived from the Madagascar periwinkle, Catharanthus roseus (formerly known as Vinca rosea), and may be selected from vincristine, vinblastine, vinorelbine and/or vindesine. In some embodiments, the one or more taxanes are selected from taxol, paclitaxel, docetaxel and/or ortataxel. In some embodiments, the one or more podophyllotoxins are selected from an etoposide and/or teniposide.

In some embodiments, the one or more topoisomerase inhibitors are selected from a type I topoisomerase inhibitor and/or a type II topoisomerase inhibitor, and may interfere with transcription and/or replication of DNA by interfering with DNA supercoiling. In some embodiments, the one or more type I topoisomerase inhibitors may comprise a camptothecin, which may be selected from exatecan, irinotecan, lurtotecan, topotecan, BNP 1350, CKD 602, DB 67 (AR67) and/or ST 1481. In some embodiments, the one or more type II topoisomerase inhibitors may comprise an epipodophyllotoxin, which may be selected from an amsacrine, etoposid, etoposide phosphate and/or teniposide.

In some embodiments, the one or more mTOR (mammalian target of rapamycin, also known as the mechanistic target of rapamycin) inhibitors are selected from rapamycin, everolimus, temsirolimus and/or deforolimus.

In some embodiments, the one or more stilbenoids are selected from resveratrol, piceatannol, pinosylvin, pterostilbene, alpha-viniferin, ampelopsin A, ampelopsin E, diptoindonesin C, diptoindonesin F, epsilon-vinferin, flexuosol A, gnetin H, hemsleyanol D, hopeaphenol, trans-diptoindonesin B, astringin, piceid and/or diptoindonesin A.

In some embodiments, the one or more STING (Stimulator of interferon genes, also known as transmembrane protein (TMEM) 173) agonists may comprise cyclic di-nucleotides, such as cAMP, cGMP, and cGAMP, and/or modified cyclic di-nucleotides that may include one or more of the following modification features: 2′-0/3′-0 linkage, phosphorothioate linkage, adenine and/or guanine analogue, and/or 2′-OH modification (e.g. protection of the 2′-OH with a methyl group or replacement of the 2′-OH by —F or —N₃).

In some embodiments, the one or more cancer vaccines are selected from an HPV vaccine, a hepatitis B vaccine, Oncophage, and/or Provenge.

In some embodiments, the one or more immunomodulatory agents may comprise an immune checkpoint inhibitor. The immune checkpoint inhibitor may target an immune checkpoint receptor, or combination of receptors comprising, for example, CTLA-4, PD-1, PD-L1, PD-L2, T cell immunoglobulin and mucin 3 (TIM3 or HAVCR2), galectin 9, phosphatidylserine, lymphocyte activation gene 3 protein (LAG3), MHC class I, MHC class II, 4-1BB, 4-1BBL, OX40, OX40L, GITR, GITRL, CD27, CD70, TNFRSF25, TL1A, CD40, CD40L, HVEM, LIGHT, BTLA, CD160, CD80, CD244, CD48, ICOS, ICOSL, B⁷-H3, B⁷-H4, VISTA, TMIGD2, HHLA2, TMIGD2, a butyrophilin (including BTNL2), a Siglec family member, TIGIT, PVR, a killer-cell immunoglobulin-like receptor, an ILT, a leukocyte immunoglobulin-like receptor, NKG2D, NKG2A, MICA, MICB, CD28, CD86, SIRPA, CD47, VEGF, neuropilin, CD30, CD39, CD73, CXCR4, and/or CXCL12.

In some embodiments, the immune checkpoint inhibitor is selected from urelumab, PF-05082566, MEDI6469, TRX518, varlilumab, CP-870893, pembrolizumab (PD1), nivolumab (PD1), atezolizumab (formerly MPDL3280A) (PD-L1), MEDI4736 (PD-L1), avelumab (PD-L1), PDR001 (PD1), BMS-986016, MGA271, lirilumab, IPH2201, emactuzumab, INCB024360, galunisertib, ulocuplumab, BKT140, bavituximab, CC-90002, bevacizumab, and/or MNRP1685A.

In some embodiments, the one or more antibiotics are selected from amikacin, gentamicin, kanamycin, neomycin, netilmicin, tobramycin, paromomycin, streptomycin, spectinomycin, geldanamycin, herbimycin, rifaximin, loracarbef, ertapenem, doripenem, imipenem, cilastatin, meropenem, cefadroxil, cefazolin, cefalotin, cefalothin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftaroline fosamil, ceftobiprole, teicoplanin, vancomycin, telavancin, dalbavancin, oritavancin, clindamycin, lincomycin, daptomycin, azithromycin, clarithromycin, dirithromycin, erythromycin, roxithromycin, troleandomycin, telithromycin, spiramycin, aztreonam, furazolidone, nitrofurantoin, linezolid, posizolid, radezolid, torezolid, amoxicillin, ampicillin, azlocillin, carbenicillin, cloxacillin, dicloxacillin, flucloxacillin, mezlocillin, methicillin, nafcillin, oxacillin, penicillin G, penicillin V, piperacillin, temocillin, ticarcillin, calvulanate, ampicillin, subbactam, tazobactam, ticarcillin, clavulanate, bacitracin, colistin, polymyxin B, ciprofloxacin, enoxacin, gatifloxacin, gemifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfacetamide, sulfadiazine, silver sulfadiazine, sulfadimethoxine, sulfamethoxazole, sulfanamide, sulfasalazine, sulfisoxazole, trimethoprim-sulfamethoxazole, sulfonamideochrysoidine, demeclocycline, minocycline, oytetracycline, tetracycline, clofazimine, dapsone, dapreomycin, cycloserine, ethambutol, ethionamide, isoniazid, pyrazinamide, rifampicin, rifabutin, rifapentine, streptomycin, arsphenamine, chloramphenicol, fosfomycin, fusidic acid, metronidazole, mupirocin, platensimycin, quinupristin, dalopristin, thiamphenicol, tigecycyline, tinidazole, trimethoprim, and/or teixobactin.

In some embodiments, the one or more antibiotics may comprise one or more cytotoxic antibiotics. In some embodiments, the one or more cytotoxic antibiotics are selected from an actinomycin, an anthracenedione, an anthracycline, thalidomide, dichloroacetic acid, nicotinic acid, 2-deoxyglucose, and/or chlofazimine. In some embodiments, the one or more actinomycins are selected from actinomycin D, bacitracin, colistin (polymyxin E) and/or polymyxin B. In some embodiments, the one or more antracenediones are selected from mitoxantrone and/or pixantrone. In some embodiments, the one or more anthracyclines are selected from bleomycin, doxorubicin (Adriamycin), daunorubicin (daunomycin), epirubicin, idarubicin, mitomycin, plicamycin and/or valrubicin.

In some embodiments, the one or more anti-fungal agents are selected from bifonazole, butoconazole, clotrimazole, econazole, ketoconazole, luliconazole, miconazole, omoconazole, oxiconazole, sertaconazole, sulconazole, tioconazole, albaconazole, efinaconazole, epoziconazole, fluconazole, isavuconazole, itraconazole, posaconazole, propiconazole, ravusconazole, terconazole, voriconazole, abafungin, amorolfin, butenafine, naftifine, terbinafine, anidulafungin, caspofungin, micafungin, benzoic acid, ciclopirox, flucytosine, 5-fluorocytosine, griseofulvin, haloprogin, tolnaflate, undecylenic acid, and/or balsam of Peru.

In some embodiments, the one or more anti-helminthic agents are selected from benzimidazoles (including albendazole, mebendazole, thiabendazole, fenbendazole, triclabendazole, and flubendazole), abamectin, diethylcarbamazine, ivermectin, suramin, pyrantel pamoate, levamisole, salicylanilides (including niclosamide and oxyclozanide), and/or nitazoxanide.

In some embodiments, other active agents are selected from growth inhibitory agents, anti-inflammatory agents (including nonsteroidal anti-inflammatory agents), anti-psoriatic agents (including anthralin and its derivatives), vitamins and vitamin-derivatives (including retinoinds, and VDR receptor ligands), corticosteroids, ion channel blockers (including potassium channel blockers), immune system regulators (including cyclosporin, FK 506, and glucocorticoids), lutenizing hormone releasing hormone agonists (such as leuprolidine, goserelin, triptorelin, histrelin, bicalutamide, flutamide and/or nilutamide), and/or hormones (including estrogen).

Unless stated otherwise, in any of the fifth to thirteenth aspects of the invention, the subject may be any human or other animal. Typically, the subject is a mammal, more typically a human or a domesticated mammal such as a cow, pig, lamb, sheep, goat, horse, cat, dog, rabbit, mouse etc. Most typically, the subject is a human.

Any of the medicaments employed in the present invention can be administered by oral, parenteral (including intravenous, subcutaneous, intramuscular, intradermal, intratracheal, intraperitoneal, intraarticular, intracranial and epidural), airway (aerosol), rectal, vaginal, occular or topical (including transdermal, buccal, mucosal, sublingual and topical occular) administration.

Typically, the mode of administration selected is that most appropriate to the disorder, disease or condition to be treated or prevented. Where one or more further active agents are administered, the mode of administration may be the same as or different to the mode of administration of the compound, salt, solvate, prodrug or pharmaceutical composition of the invention.

For oral administration, the compounds, salts, solvates or prodrugs of the present invention will generally be provided in the form of tablets, capsules, hard or soft gelatine capsules, caplets, troches or lozenges, as a powder or granules, or as an aqueous solution, suspension or dispersion.

Tablets for oral use may include the active ingredient mixed with pharmaceutically acceptable excipients such as inert diluents, disintegrating agents, binding agents, lubricating agents, sweetening agents, flavouring agents, colouring agents and preservatives. Suitable inert diluents include sodium and calcium carbonate, sodium and calcium phosphate, and lactose. Corn starch and alginic acid are suitable disintegrating agents. Binding agents may include starch and gelatine. The lubricating agent, if present, may be magnesium stearate, stearic acid or talc. If desired, the tablets may be coated with a material, such as glyceryl monostearate or glyceryl distearate, to delay absorption in the gastrointestinal tract. Tablets may also be effervescent and/or dissolving tablets.

Capsules for oral use include hard gelatine capsules in which the active ingredient is mixed with a solid diluent, and soft gelatine capsules wherein the active ingredient is mixed with water or an oil such as peanut oil, liquid paraffin or olive oil.

Powders or granules for oral use may be provided in sachets or tubs. Aqueous solutions, suspensions or dispersions may be prepared by the addition of water to powders, granules or tablets.

Any form suitable for oral administration may optionally include sweetening agents such as sugar, flavouring agents, colouring agents and/or preservatives.

Formulations for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active ingredient such carriers as are known in the art to be appropriate.

For parenteral use, the compounds, salts, solvates or prodrugs of the present invention will generally be provided in a sterile aqueous solution or suspension, buffered to an appropriate pH and isotonicity. Suitable aqueous vehicles include Ringer's solution and isotonic sodium chloride or glucose. Aqueous suspensions according to the invention may include suspending agents such as cellulose derivatives, sodium alginate, polyvinylpyrrolidone and gum tragacanth, and a wetting agent such as lecithin. Suitable preservatives for aqueous suspensions include ethyl and n-propyl p-hydroxybenzoate. The compounds of the invention may also be presented as liposome formulations.

For ocular administration, the compounds, salts, solvates or prodrugs of the invention will generally be provided in a form suitable for topical administration, e.g. as eye drops. Suitable forms may include ophthalmic solutions, gel-forming solutions, sterile powders for reconstitution, ophthalmic suspensions, ophthalmic ointments, ophthalmic emulsions, ophthalmic gels and ocular inserts. Alternatively, the compounds, salts, solvates or prodrugs of the invention may be provided in a form suitable for other types of ocular administration, for example as intraocular preparations (including as irrigating solutions, as intraocular, intravitreal or juxtascleral injection formulations, or as intravitreal implants), as packs or corneal shields, as intracameral, subconjunctival or retrobulbar injection formulations, or as iontophoresis formulations.

For transdermal and other topical administration, the compounds, salts, solvates or prodrugs of the invention will generally be provided in the form of ointments, cataplasms (poultices), pastes, powders, dressings, creams, plasters or patches.

Suitable suspensions and solutions can be used in inhalers for airway (aerosol) administration.

The dose of the compounds, salts, solvates or prodrugs of the present invention will, of course, vary with the disorder, condition or disease to be treated or prevented. In general, a suitable dose will be in the range of 0.01 to 500 mg per kilogram body weight of the recipient per day. The desired dose may be presented at an appropriate interval such as once every other day, once a day, twice a day, three times a day or four times a day. The desired dose may be administered in unit dosage form, for example, containing 1 mg to 50 g of active ingredient per unit dosage form.

A fourteenth aspect of the invention relates to the use of a compound of the first or second aspect of the present invention, or a salt thereof, as an intermediate to prepare another compound of the first or second aspect of the present invention, or a salt thereof. For example, compounds of the first aspect of the invention where J is —S— may be used to prepare compounds of the invention where J is —SO—, —SO₂— or —SO(═NR^{jj})—. Likewise, compounds of the first aspect of the invention where J is —S—C(R^{j})₂— may be used to prepare compounds of the invention where J is —SO—C(R^{j})₂—, —SO₂—C(R^{jj})₂—, or —SO(═NR^{jj})—C(R^{j})₂—. Similarly, compounds of the first aspect of the invention where J is —SO(═NR^{jj})— or —SO(═NR^{jj})—C(R^{j})₂— and R^{jj} is a protecting group may be used to prepare compounds of the invention where J is —SO(═NH)— or —SO(═NH)—C(R^{j})₂—.

For the avoidance of doubt, insofar as is practicable any embodiment of a given aspect of the present invention may occur in combination with any other embodiment of the same aspect of the present invention. In addition, insofar as is practicable it is to be understood that any preferred, typical or optional embodiment of any aspect of the present invention should also be considered as a preferred, typical or optional embodiment of any other aspect of the present invention.

### EXAMPLES—COMPOUND SYNTHESIS

All solvents, reagents and compounds were purchased and used without further purification unless stated otherwise.

### Abbreviations

AcOH acetic acid
app apparent
aq aqueous
B₂Pin₂ bis(pinacolato)diboron
Boc tert-butyloxycarbonyl
br broad
Cbz carboxybenzyl
CDI 1,1-carbonyl-diimidazole
conc concentrated
m-CPBA 3-chlorobenzoperoxoic acid
d doublet
DCM dichloromethane
dd double doublet
DBU 1,8-diazabicyclo[5.4.0]undec-7-ene
DIPEA diisopropylethylamine
DMA N,N-dimethylacetamide
DMAP N,N-dimethylpyridin-4-amine
DMF N,N-dimethylformamide
DMSO dimethylsulfoxide
dt/td double triplet/triple doublet
(ES+)/(ES⁻) electrospray ionization, positive/negative mode
Et ethyl
EtOAc ethyl acetate
EtOH ethanol
h hour(s)
HATU 1-[bis(dimethylamino)methylene]-1H-1,2,3-triazolo[4,5-b]pyridinium 3-oxid hexafluorophosphate
HMBC-NMR Heteronuclear multiple-bond correlation NMR
HPLC high performance liquid chromatography (reverse phase)
LC liquid chromatography
LiHMDS Lithium bis(trimethylsilyl)amide
m multiplet
(M+H)⁺ protonated molecular ion
Me methyl
MeCN acetonitrile
MeOH methanol
MHz megahertz
min minute(s)
Ms methanesulfonyl
MS mass spectrometry
MTBE/TBME methyl tert-butyl ether
m/z mass-to-charge ratio
NBS 1-bromopyrrolidine-2,5-dione
NCS 1-chloropyrrolidine-2,5-dione
NMP N-methylpyrrolidine
NMR nuclear magnetic resonance (spectroscopy)
Oxone potassium peroxymonosulfate
p pentuplet
Pd-175 [tBuBrettPhosPd(allyl)]OTf: (allyl(2-di-tert-butylphosphino-2′,4′,6′-triisopropyl-3,6-dimethoxy-1,1′-biphenyl)palladium(II) triflate) from Johnson Matthey
Pd(dba)₂ bis(dibenzylideneacetone)palladium(0)
Pd₂(dba)₃ tris(dibenzylideneacetone)dipalladium(0)
Pd(dppf)Cl₂ [1,1′-bis(diphenylphosphino)ferrocene]dichloropalladium(II)
PE petroleum ether
Ph phenyl
PMB 4-methoxybenzyl
prep-HPLC preparative-high performance liquid chromatography
prep-TLC preparative-thin layer chromatography
q quartet
RP reverse phase
RT room temperature
s singlet
sat saturated
SCX solid supported cation exchange (resin)
SEM 2-(trimethylsilyl)ethyoxy methyl
sept septuplet
t triplet
TBAF tetrabutylammonium fluoride
TEA triethylamine
TFA trifluoroaceticacid
THF tetrahydrofuran
TLC thin layer chromatography
TMS trimethylsilyl
XantPhos (9,9-dimethyl-9H-xanthene-4,5-diyl)bis(diphenylphosphine)
XPhos 2-dicyclohexylphosphino-2′,4′,6′-triisopropylbiphenyl

### EXPERIMENTAL METHODS

Nuclear Magnetic Resonance

NMR spectra were recorded at 300, 400 or 500 MHz. Spectra were measured at 298 K, unless indicated otherwise, and were referenced relative to the solvent resonance. The chemical shifts are reported in parts per million. Spectra were recorded using one of the following machines:
a Bruker 400 MHz spectrometer using ICON-NMR, under TopSpin program control,
a Bruker Avance III spectrometer at 400 MHz fitted with a BBO 5 mm liquid probe, or
a Bruker Avance III HD spectrometer at 500 MHz, equipped with a Bruker 5 mm SmartProbe™.

LC-MS

LC-MS Methods: Using SHIMADZU LCMS-2020, Agilent 1200 LC/G1956A MSD and Agilent 1200\G6110A, Agilent 1200 LC and Agilent 6110 MSD. Mobile Phase: A: 0.025% NH₃.H₂O in water (v/v); B: acetonitrile. Column: Kinetex EVO C18 2.1×30 mm, 5 μm.

Reversed Phase HPLC Conditions for the LCMS Analytical Methods

Methods 1a and 1b: Waters Xselect CSH C18 XP column (4.6×30 mm, 2.5 μm) at 40° C.; flow rate 2.5-4.5 mL min-1 eluted with a H₂O-MeCN gradient containing either 0.1% v/v formic acid (Method 1a) or 10 mM NH₄HCO₃ in water (Method 1b) over 4 min employing UV detection at 254 nm.

Method 1c: Agilent 1290 series with UV detector and HP 6130 MSD mass detector using Waters XBridge BEH C18 XP column (2.1×50 mm, 2.5 μm) at 35° C.; flow rate 0.6 mL/min; mobile phase A: ammonium acetate (10 mM); water/MeOH/acetonitrile (900:60:40); mobile phase B: ammonium acetate (10 mM); water/MeOH/acetonitrile (100:540:360); over 4 min employing UV detection at 215 and 238 nm.

Reversed Phase HPLC Conditions for the UPLC Analytical Methods

Methods 2a and 2b: Waters BEH C18 (2.1×30 mm, 1.7 μm) at 40° C.; flow rate 0.77 mL min-1 eluted with a H₂O-MeCN gradient containing either 0.1% v/v formic acid (Method 2a) or 10 mM NH₄HCO₃ in water (Method 2b) over 3 min employing UV detection at 254 nm.

Reversed Phase HPLC Purification

Automated reversed phase column chromatography was carried out using:

(i) a Gilson GX-281 system driven by a Gilson-322 pump module, Gilson-156 UV photometer detection unit and Gilson-281 fraction collector. Detection wavelength: 215 nm, 220 nm and 254 nm; or

(ii) a Gilson GX-215 system driven by a LC-20AP pump module, SPD-20A UV photometer detection unit and Gilson-215 fraction collector. Detection wavelength: 215 nm, 220 nm and 254 nm; or

(iii) a Shimadzu CBM-20A system driven by LC-20AP pump module, SPD-20A UV photometer detection unit and FRC-10A fraction collector. Detection wavelength: 215 nm, 220 nm and 254 nm; or

(iv) a TELEDYNE ISCO CombiFlash R^{f}+150. Detection wavelength: 215 nm, 220 nm and 254 nm.

Preparative Reversed Phase HPLC General Methods

Acidic prep HPLC (x-y % MeCN in water): Waters X-Select CSH column C18.5 μm (19×50 mm), flow rate 28 mL min-1 eluting with a H₂O-MeCN gradient containing 0.1% v/v formic acid over 6.5 min using UV detection at 254 nm. Gradient information: 0.0-0.2 min, x % MeCN; 0.2-5.5 min, ramped from x % MeCN to y % MeCN; 5.5-5.6 min, ramped from y % MeCN to 95% MeCN; 5.6-6.5 min, held at 95% MeCN.

Acidic prep HPLC (x-y % MeOH in water): Waters X-Select CSH column C18.5 μm (19×50 mm), flow rate 28 mL min-1 eluting with a 10 mM formic acid-MeOH gradient over 7.5 min using UV detection at 254 nm. Gradient information: 0.0-1.5 min, x % MeOH; 1.5-6.8 min, ramped from x % MeOH to y % MeOH; 6.8-6.9 min, ramped from y % MeOH to 95% MeOH; 6.9-7.5 min, held at 95% MeOH.

Basic prep HPLC (x-y % MeCN in water): Waters X-Bridge Prep column C18.5 μm (19×50 mm), flow rate 28 mL min-1 eluting with a 10 mM NH₄HCO₃-MeCN gradient over 6.5 min using UV detection at 254 nm. Gradient information: 0.0-0.2 min, x % MeCN; 0.2-5.5 min, ramped from x % MeCN to y % MeCN; 5.5-5.6 min, ramped from y % MeCN to 95% MeCN; 5.6-6.5 min, held at 95% MeCN.

### Synthesis of Intermediates

### Intermediate L1: 1-(3-bromo-1-methyl-1H-pyrazol-5-yl)-N,N-dimethylethanamine

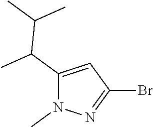

### Step A: tert-butyl (1-methyl-1H-pyrazol-3-yl)carbamate

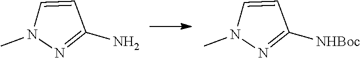

To a solution of 1-methyl-1H-pyrazol-3-amine (40 g, 411.87 mmol, 1 eq) in THF (400 mL) was added a solution of NaOH (18.12 g, 453.06 mmol, 1.1 eq) in H₂O (400 mL) and Boc₂O (107.87 g, 494.24 mmol, 1.2 eq). The reaction mixture was stirred at 25° C. for 12 hours. The reaction mixture was diluted with H₂O (500 mL) and extracted with EtOAc (3×500 mL). The combined organic layers were washed with brine (2×200 mL), dried over Na₂SO₄, filtered and concentrated under reduced pressure. The residue was triturated with MTBE (200 mL) to give the title compound (36 g, 4432% yield, 100% purity on LCMS) as a white solid.

¹H NMR (400 MHz, CDCl₃): δ 8.59 (br s, 1H), 7.19 (d, 1H), 6.45 (s, 1H), 3.81 (s, 3H) and 1.50 (s, 9H).

LCMS: m/z 220.1 (M+Na)⁺ (ES⁺).

### Step B: tert-butyl (5-(1-hydroxyethyl)-1-methyl-1H-pyrazol-3-yl)carbamate

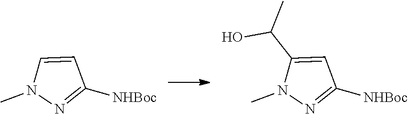 [SMILES: Cn1ccc(NC(=O)OC(C)(C)C)n1 ; CC(O)c1cc(NC(=O)OC(C)(C)C)nn1C]

A solution of tert-butyl (1-methyl-1H-pyrazol-3-yl)carbamate (18 g, 91.26 mmol, 1 eq) in THF (400 mL) was cooled to −75° C., then n-BuLi (2.5 M, 80.31 mL, 2.2 eq) was added dropwise into the above mixture at −75° C. The reaction mixture was stirred at −75° C. for 1 hour. Then CH₃CHO (8.04 g, 182-52 mmol, 2 eq) was added into the above mixture. The resulting mixture was warmed to 25° C. and stirred at 25° C. for 1 hour. The reaction mixture was quenched with H₂O (500 mL) at 25° C. and extracted with EtOAc (3×500 mL). The combined organic layers were washed with brine (2×200 mL), dried over Na₂SO₄, filtered and concentrated under reduced pressure. The residue was purified by silica gel column chromatography (SiO₂, Petroleum ether:Ethyl acetate, 10:1 to 0:1) to give the title compound (11 g, 50% yield, 90% purity on ¹H NMR) as a white solid.

¹H NMR (400 MHz, CDCl₃): δ 8.07 (s, 1H), 6.41 (s, 1H), 4.83-4.81 (m, 1H), 3.78 (s, 3H), 1.56 (d, 3H) and 1.47 (s, 9H). One exchangeable proton not observed.

LCMS: 264.1 m/z (M+Na)⁺ (ES⁺).

### Step C: 1-(3-amino-1-methyl-1H-pyrazol-5-yl)ethanol

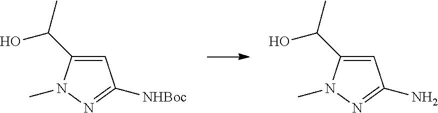

To a solution of tert-butyl (5-(1-hydroxyethyl)-1-methyl-H-pyrazol-3-yl)carbamate (27 g, 111.90 mmol, 1 eq) in DCM (50 mL) was added HCl/EtOAc (4 M, 720 mL, 25.74 eq). The reaction mixture was stirred at 25° C. for 12 hours. The reaction mixture was concentrated in vacuum to give the title compound (25 g, crude, HCl salt), which was used in next step directly without further purification.

¹H NMR (400 MHz, CDCl₃): δ 5.51 (s, 1H), 4.83-4.77 (m, 1H), 3.68 (s, 3H) and 1.52 (d, 3H). Three exchangable protons not observed.

LCMS: 142.2 m/z (M+H)⁺ (ES⁺).

### Step D: 1-(3-bromo-1-methyl-1H-pyrazol-5-yl)ethanol

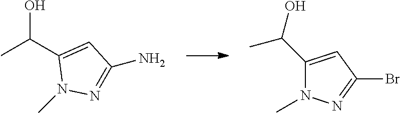

To a solution of 1-(3-amino-1-methyl-1H-pyrazol-5-yl)ethanol (5.0, 35.42 mmol, 1 eq) in MeCN (100 mL) was added tert-butyl nitrite (5.48 g, 53-13 mmol, 1.5 eq), CuBr (254 mg, 1.77 mmol, 0.05 eq) and CuBr₂ (9.49 g, 42-50 mmol, 1.2 eq). The reaction mixture was heated to 60° C. and stirred at 60° C. for 16 hours under N₂. The mixture was diluted with H₂O (100 mL) and extracted with EtOAc (3×80 mL). The combined organic layers were dried over Na₂SO₄, filtered and concentrated in vacuum. The residue was purified by reverse phase flash chromatography (0.1% NH₃.H₂O/MeCN) to give the title compound (2.0 g, 43.6% yield over two steps, 94% purity on LCMS) as a red oil. ¹H NMR (400 MHz, CDCl₃): δ 6.19 (s, 1H), 4.92-4.84 (m, 1H), 3.88 (s, 3H) and 1.57 (d, 3H). One exchangable proton not observed.

LCMS: m/z 205.2 (M+H)⁺ (ES⁺).

### Step E: 1-(3-bromo-1-methyl-1H-pyrazol-5-yl)ethyl methanesulfonate

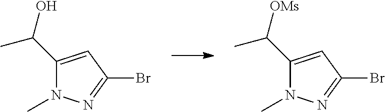

To a solution of 1-(3-bromo-1-methyl-1H-pyrazol-5-yl)ethanol (1.89 g, 9.22 mmol, 1 eq) and DIPEA (2.38 g, 18.43 mmol, 2.0 eq) in DCM (40 mL) was added MsCl (1.58 g, 13.83 mmol, 1.5 eq) at 0° C. The reaction mixture was warmed to 15° C. and stirred at 15° C. for 30 minutes. The mixture was diluted with H₂O (50 mL) and extracted with DCM (2×30 mL. The combined organic layers were dried over NaSO₄. filtered and concentrated in vacuum to give the title compound (2.0 g, crude) as a brown oil, which was used directly for the next step.

### Step F: 1-(3-bromo-1-methyl-1H-pyrazol-5-yl)-N,N-dimethylethanamine

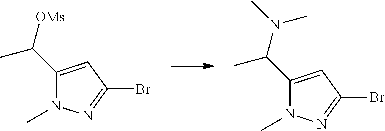

A solution of 1-(3-bromo-1-methyl-1H-pyrazol-5-yl)ethyl methanesulfonate (0.69 g, 2.44 mmol, 1 eq) in Me₂NH (2 M, 10 mL, THF solution) was stirred at 60° C. for 16 hours. The reaction mixture was concentrated in vaccum. The residue was purified by reverse phase flash chromatography (0.1% NH₃.H₂O/MeCN) to give the title compound (0.57 g, 77.2% yield over two steps, 99% purity on LCMS) as a brown oil.

¹H NMR (400 MHz, CDCl₃): δ 6.10 (s, 1H), 3.85 (s, 3H), 3.69-3.64 (m, 1H), 2.19 (s, 6H) and 1.28 (d, 3H).

LCMS: m/z 232.2 (M+H)⁺ (ES⁺).

### Intermediate L2: 1-(3-bromo-1-isopropyl-1H-pyrazol-5-yl)-N,N-dimethylethanamine

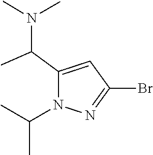

### Step A: 1-isopropyl-3-nitro-1H-pyrazole

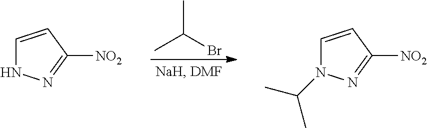

To a solution of 3-nitro-1H-pyrazole (25 g, 221.09 mmol, 1 eq) in DMF (300 mL) was added NaH (10.61 g, 265.31 mmol, 60 wt. % in mineral oil, 1.2 eq) at 0° C. under N₂. The suspension was stirred at 0° C. for 0.5 hour, then 2-bromopropane (27.19 g, 221.09 mmol, 1 eq) was added dropwise to the reaction mixture at 0° C. The reaction mixture was stirred at 15° C. for 12 hours. The reaction mixture was quenched with saturated aqueous NH₄Cl solution (200 mL), diluted with water (500 mL) and extracted with EtOAc (3×500 mL). The organic phases were washed with brine (500 mL), dried over anhydrous Na₂SO₄ filtered and concentrated in vacuum. The residue was purified by column chromotography (SiO₂, Petroleum ether:Ethyl acetate, 1:0 to 5:1) to give the title compound (15 g, 4373% yield) as a yellow oil.

¹H NMR (400 MHz, CDCl₃): δ 7.49 (d, 1H), 6.89 (d, 1H), 4.65-4.54 (m, 1H) and 1.57 (d, 6H).

### Step B: 1-isopropyl-1H-pyrazol-3-amine

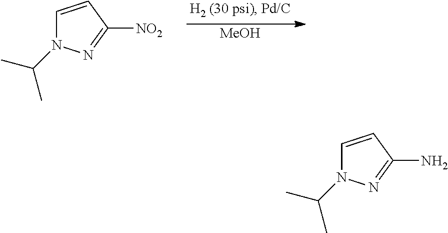

To a solution of 1-isopropyl-3-nitro-pyrazole (15 g, 96.68 mmol, 1 eq) in MeOH (150 mL) was added Pd/C (3 g, 10 wt. % loading on activated carbon) under N₂. The reaction mixture was stirred at 25° C. for 12 hours under H₂ (30 psi). The reaction mixture was filtered and the filtrate was concentrated in vacuum. The residue was purified by silica gel column chromatography (Petroleum ether:Ethyl acetate, 30:1 to 2:1) to give the title compound (11 g, 90.90% yield) as a brown oil.

¹H NMR (400 MHz, CDCl₃): δ 7.16 (d, 1H), 5.57 (d, 1H), 4.32-4.21 (m, 1H), 3.60 (br s, 2H) and 1.44 (d, 6H).

### Step C: tert-butyl (1-isopropyl-1H-pyrazol-3-yl)carbamate

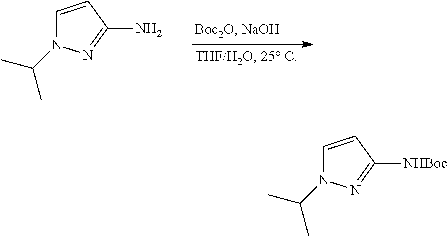

To a solution of 1-isopropyl-1H-pyrazol-3-amine (11 g, 87.88 mmol, 1 eq) in THF (80 mL) was added a solution of NaOH (4.22 g, 105.46 mmol, 1.2 eq) in H₂O (80 mL), and then Boc₂O (23.02 g, 105.46 mmol, 1.2 eq) was added into the above mixture. The reaction mixture was stirred at 25° C. for 12 hours. The reaction mixture was diluted with H₂O (200 mL) and extracted with EtOAc (3×200 mL). The organic phase was washed with brine (2×150 mL), dried over anhydrous Na₂SO₄, filtered and concentrated in vacuum. The residue was triturated with MTBE (200 mL) to give the title compound (14.8 g, 74.75% yield) as a white solid.

¹H NMR (400 MHz, CDCl₃): δ 7.28 (d, 1H), 7.05 (s, 1H), 6.41 (s, 1H), 4.40-431 (m, 1H), 1.53 (s, 9H) and 1.45 (d, 6H).

LCMS: m/z 473.4 (2M+Na)⁺ (ES⁺).

### Step D: tert-butyl (5-(1-hydroxyethyl)-1-isopropyl-1H-pyrazol-3-yl)carbamate

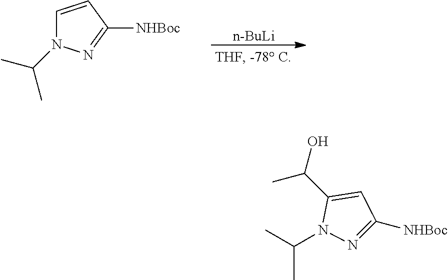

To a solution of tert-butyl (1-isopropyl-1H-pyrazol-3-yl)carbamate (4 g, 17.76 mmol, 1 eq) in THF (100 mL) was added n-BuLi (2.5 M, 15.62 mL, 2.2 eq) dropwise at −68° C. under N₂ atmosphere. The mixture was stirred at −68° C. for 1 hour. Then a solution of acetaldehyde (15.64 g, 355.10 mmol, 20 eq) in THF (60 mL) was added to the above reaction mixture at −68° C. The reaction mixture was stirred at −68° C. for 1 hour. The reaction mixture was quenched with saturated aqueous NH₄Cl solution (100 mL) and extracted with ethyl acetate (300 mL). The organic layer was washed with brine (100 mL), dried over Na₂SO₄, filtered and concentrated in vacuum. The residue was purified by silica gel column chromatography (SiO₂, Petroleum ether:ethyl acetate, 1:0 to 10:1, then flushed through with Petroleum ether:Ethyl acetate:Dichloromethane, 3:1:1) to give the title compound (2.85 g, 29.80% yield) as a yellow oil.

¹H NMR (400 MHz, CDCl₃): δ 7.19 (s, 1H), 6.36 (s, 1H), 4.86-4.84 (m, 1H), 4.73-457 (m, 1H), 1.57 (d, 3H), 1.47 (s, 9H) and 1.38 (dd, 6H). One exchangable proton not observed

### Step E: 1-(3-((tert-butoxycarbonyl)amino)-1-isopropyl-1H-pyrazol-5-yl)ethyl methanesulfonate

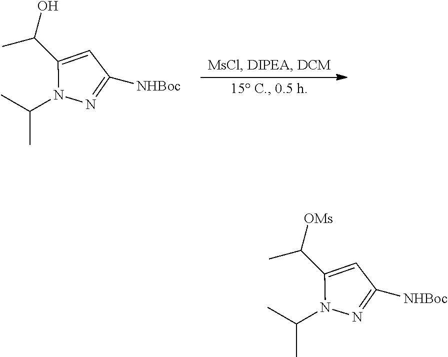 [SMILES: CC(O)c1cc(NC(=O)OC(C)(C)C)nn1C(C)C ; CC(OS(C)(=O)=O)c1cc(NC(=O)OC(C)(C)C)nn1C(C)C]

To a solution of tert-butyl (5-(1-hydroxyethyl)-1-isopropyl-1H-pyrazol-3-yl)carbamate (1.7 g, 6.31 mmol, 1 eq) and DIPEA (1.63 g, 12.62 mmol, 2 eq) in DCM (40 mL) was added MsCl (1.08 g, 9.47 mmol, 1.5 eq) at 0° C. The mixture was stirred at 15° C. for 0.5 hour. The reaction mixture was quenched with water (50 mL) and extracted with dichloromethane (10 mL). The organic phase was washed with brine, dried over anhydrous Na₂SO₄, filtered and concentrated in vacuum to give the title compound (2.0 g, crude) as a yellow oil, which was used in the next step without purification.

### Step F: tert-butyl (5-(1-(dimethylamino)ethyl)-1-isopropyl-1H-pyrazol-3-yl)carbamate

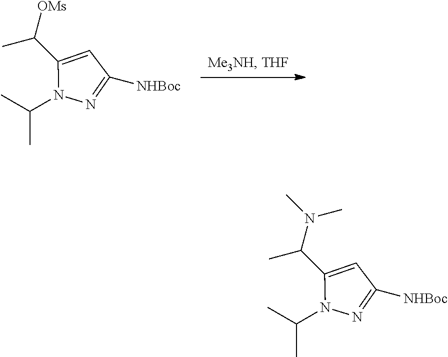 [SMILES: CC(OS(C)(=O)=O)c1cc(NC(=O)OC(C)(C)C)nn1C(C)C ; CC(c1cc(NC(=O)OC(C)(C)C)nn1C(C)C)N(C)C]

A solution of 1-(3-((tert-butoxycarbonyl)amino)-1-isopropyl-1H-pyrazol-5-yl)ethyl methanesulfonate (2 g, 5.76 mmol, 1 eq) in dimethylamine (2 M, 18.75 mL, 6.51 eq, THF solution) was stirred at 60° C. for 18 hours. The reaction mixture was concentrated in vacuum. The residue was purified by reverse phase flash chromatography (0.1% ammonium hydroxide/MeCN) to give the title compound (1.2 g, 64.1% yield over two steps) as a yellow oil.

¹H NMR (400 MHz, CDCl₃): δ 6.94 (s, 1H), 6.29 (s, 1H), 4.76-4.62 (m, 1H), 3.75-3.69 (m, 1H), 2.20 (s, 6H), 1.50 (s, 9H), 1.40 (d, 3H) and 1.34 (dd, 6H).

### Step G: 5-(1-(dimethylamino)ethyl)-1-isopropyl-1H-pyrazol-3-amine

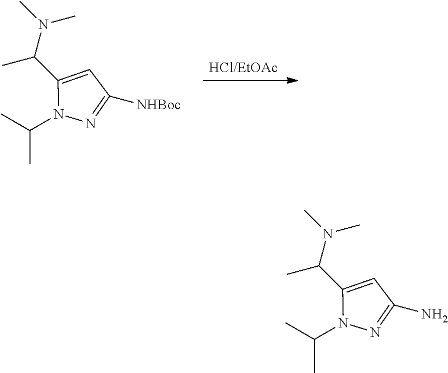 [SMILES: CC(c1cc(NC(=O)OC(C)(C)C)nn1C(C)C)N(C)C ; CC(c1cc(N)nn1C(C)C)N(C)C]

A solution of tert-butyl (5-(1-(dimethylamino)ethyl)-1-isopropyl-1H-pyrazol-3-yl)carbamate (1.2 g, 4.05 mmol, 1 eq) in HCl/EtOAc (4 M, 20 mL) was stirred at 15° C. for 24 hours. The reaction mixture was concentrated in vacuum to give the title compound (1 g, crude, HCl salt) as a yellow solid.

¹H NMR (400 MHz, DMSO-d₆): δ 6.62 (s, 1H), 4.93-4.83 (m, 2H), 2.76 (s, 3H), 2.59 (s, 3H), 1.63 (d, 3H), 1.46 (d, 3H) and 1.29 (d, 3H). Two exchangable protons not observed.

### Step H: 1-(3-bromo-1-isopropyl-1H-pyrazol-5-yl)-N,N-dimethylethanamine

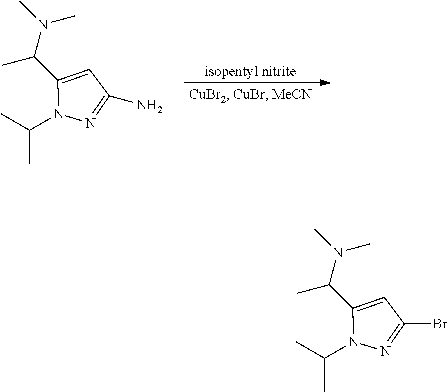

To a solution of 5-(1-(dimethylamino)ethyl)-1-isopropyl-1H-pyrazol-3-amine (1 g, 5.09 mmol, 1 eq, HCl salt) in MeCN (20 mL) were added CuBr (37 mg, 254.72 μmol, 0.05 eq) and CuBr₂ (683 mg, 3.06 mmol, 0.6 eq). Then isopentyl nitrite (895 mg, 7.64 mmol, 1.03 mL, 1.5 eq) was added to the mixture. The reaction mixture was stirred at 60° C. for 1 hour. The reaction mixture was diluted with saturated aqueous NaHCO₃ solution (100 mL) and extracted with ethyl acetate (100 mL). The organic layer was separated and washed with brine (100 mL), dried over anhydrous Na₂SO₄, filtered and concentrated in vacuum. The residue was purified by silica gel column chromatography (Petroleum ether:Ethyl acetate, 10:1 to 5:1) to give the title compound (520 mg, 49.4% yield over two steps) as a yellow oil.

¹H NMR (400 MHz, CDCl₃): δ 6.06 (s, 1H), 4.82-4.70 (m, 1H), 3.72-3.67 (m, 1H), 2.20 (s, 6H), 1.48-1.42 (m, 6H) and 1.29 (d, 3H).

### Intermediate L5: 1-(3-bromo-1-cyclopropyl-1H-pyrazol-5-yl)-N,N-dimethylethanamine

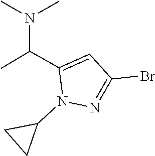

### Step A: 1-cyclopropyl-3-nitro-1H-pyrazole

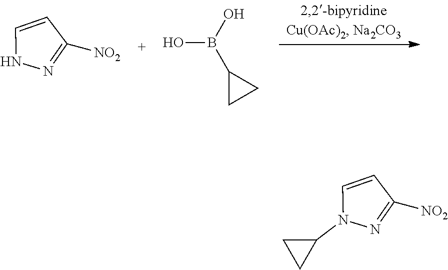 [SMILES: O=[N+]([O-])c1cc[nH]n1 ; OB(O)C1CC1 ; O=[N+]([O-])c1ccn(C2CC2)n1]

To a solution of cyclopropylboronic acid (20.89 g, 243.20 mmol, 1.1 eq) in dioxane (400 mL) were added 3-nitro-1H-pyrazole (25 g, 221.09 mmol, 1 eq), 2,2′-bipyridine (3453 g, 221.09 mmol, 1 eq) and Na₂CO₃ (35.15 g, 331.64 mmol, 1.5 eq). The reaction mixture was stirred at 25° C. for 0.5 hour. Then Cu(OAc)₂ (40.16 g, 221.09 mmol, 1 eq) was added into the above mixture and the resulting mixture was warmed to 70° C. and stirred at 70° C. for 11.5 hours. The reaction mixture was filtered and the filtrate was concentrated under reduced pressure. The residue was purified by silica gel column chromatography (SiO₂, Petroleum ether:Ethyl acetate, 50:1 to 5:1) to give the title compound (16.3 g, 48.14% yield) as a yellow oil.

¹H NMR (400 MHz, CDCl₃): δ 7.53 (d, 1H), 6.86 (d, 1H), 3.74-3.68 (m, 1H) and 1.30-1.21 (m, 4H).

### Step B: 1-cyclopropyl-1H-pyrazol-3-amine

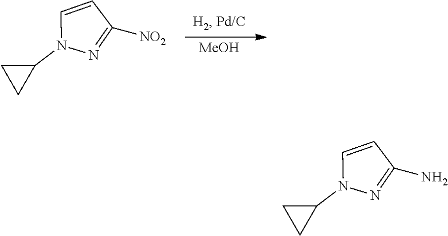 [SMILES: "MeOH" ; O=[N+]([O-])c1ccn(C2CC2)n1 ; Nc1ccn(C2CC2)n1]

To a solution of 1-cyclopropyl-3-nitro-1H-pyrazole (16 g, 104.48 mmol, 1 eq) in MeOH (100 mL) was added Pd/C (2 g, 10 wt. % loading on activated carbon) under N₂. The suspension was degassed in vacuum and purged with H₂ several times. The reaction mixture was stirred at 30° C. for 12 hours under H₂ (40 psi). The reaction mixture was filtered and the filtrate was concentrated in vacuum to give the title compound (11.8 g, 91.70% yield) as a yellow oil.

¹H NMR (400 MHz, CDCl₃): δ 7.16 (d, 1H), 5.54 (d, 1H), 3.61 (br s, 2H), 3.41-335 (m, 1H), 1.02-0.99 (m, 2H) and 0.93-0.85 (m, 2H).

LCMS: m/z 124.2 (M+H)⁺ (ES⁺).

### Step C: tert-butyl (1-cyclopropyl-1H-pyrazol-3-yl)carbamate

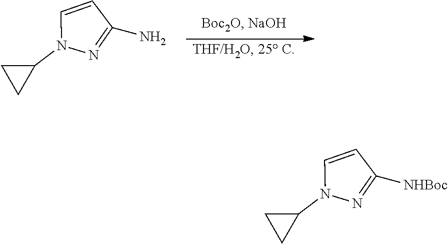 [SMILES: Nc1ccn(C2CC2)n1 ; "THF/H2O, 25° C." ; CC(C)(C)OC(=O)Nc1ccn(C2CC2)n1]

To a mixture of 1-cyclopropyl-1H-pyrazol-3-amine (11.6 g, 94.19 mmol, 1 eq) in THF (100 mL) was added a solution of NaOH (4.52 g, 113.03 mmol, 1.2 eq) in H₂O (100 mL). Then Boc₂O (30.84 g, 141.28 mmol, 32.46 mL, 1.5 eq) was added into the above mixture. The reaction mixture was stirred at 25° C. for 12 hours. The reaction mixture was diluted with H₂O (100 mL) and extracted with EtOAc (2×200 mL). Then the organic layers were washed with brine (2×100 mL), dried over Na₂SO₄, filtered and concentrated in vacuum. The residue was purified by column chromatography (SiO₂, Petroleum ether:Ethyl acetate, 30:1 to 0:1) to give the title compound (16.5 g, 78.46% yield) as a yellow oil.

¹H NMR (400 MHz, CDCl₃): δ 7.29 (d, 1H), 7.03 (s, 1H), 6.41-6.38 (m, 1H), 3.50-343 (m, 1H), 1.50 (s, 9H), 1.03-1.01 (m, 2H) and 0.98-0.94 (m, 2H).

LCMS: m/z 246.2 (M+Na)⁺ (ES⁺).

### Step D: tert-butyl (1-cyclopropyl-5-(1-hydroxyethyl)-1H-pyrazol-3 yl)carbamate

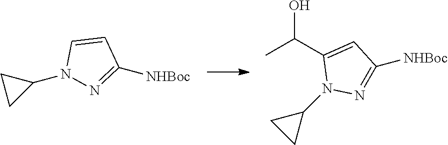 [SMILES: CC(C)(C)OC(=O)Nc1ccn(C2CC2)n1 ; CC(O)c1cc(NC(=O)OC(C)(C)C)nn1C1CC1]

To a solution of tert-butyl (1-cyclopropyl-1H-pyrazol-3-yl)carbamate (22.6 g, 101.22 mmol, 1 eq) in THF (200 mL) was added n-BuLi (2.5 M, 89.08 mL, 2.2 eq) dropwise at −75° C. The reaction mixture was stirred at −75° C. for 1 hour. Then CH₃CHO (13.36 g, 303.67 mmol, 3 eq) was added into the mixture. The reaction mixture was warmed to 25° C. and stirred at 25° C. for 12 hours. The reaction mixture was quenched with H₂O (100 mL) at 0° C., diluted with water (2×100 mL) and extracted with EtOAc (3×200 mL). The organic phase was washed with brine (200 mL), dried over anhydrous Na₂SO₄, filtered and concentrated in vacuum. The residue was purified by silica gel column chromatography (SiO₂, Petroleum ether:Ethyl acetate, 10:1 to 1:1) to give the title compound (18 g, 66.52% yield) as a white solid.

¹H NMR (400 MHz, CDCl₃): δ 7.00 (s, 1H), 6.45 (s, 1H), 5.13-5.04 (m, 1H), 3.48-3.42 (m, 1H), 1.60 (d, 3H), 1.49 (s, 9H), 1.27-1.22 (m, 2H) and 1.02-0.97 (m, 2H). One exchangable proton not observed.

### Step E: 1-(3-((tert-butoxycarbonyl)amino)-1-cyclopropyl-1H-pyrazol-5-yl)ethyl methanesulfonate

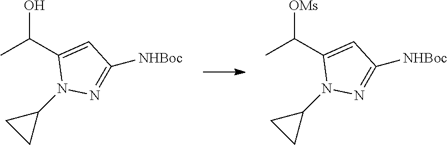 [SMILES: CC(O)c1cc(NC(=O)OC(C)(C)C)nn1C1CC1 ; CC(OS(C)(=O)=O)c1cc(NC(=O)OC(C)(C)C)nn1C1CC1]

To a solution of tert-butyl (1-cyclopropyl-5-(1-hydroxyethyl)-1H-pyrazol-3-yl)carbamate (10 g, 37.41 mmol, 1 eq) in DCM (150 mL) was added TEA (7.57 g, 74.82 mmol, 2 eq) and MsCl (6.85 g, 59.80 mmol, 1.60 eq) at 0° C. The mixture was stirred at 25° C. for 1 hour. The reaction mixture was diluted with water (200 mL) and extracted with DCM (3×200 mL). The organic phase was washed with brine (200 mL), dried over anhydrous Na₂SO₄, filtered and concentrated in vacuum to give the title compound (11 g, crude) as a yellow solid, which was used to the next step directly without further purification.

### Step F: tert-butyl (1-cyclopropyl-5-(1-(dimethylamino)ethyl)-1H-pyrazol-3-yl)carbamate

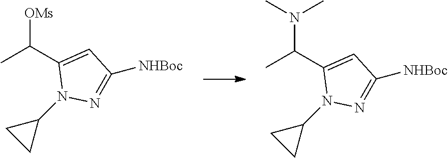 [SMILES: CC(OS(C)(=O)=O)c1cc(NC(=O)OC(C)(C)C)nn1C1CC1 ; CC(c1cc(NC(=O)OC(C)(C)C)nn1C1CC1)N(C)C]

A mixture of 1-(3-((tert-butoxycarbonyl)amino)-1-cyclopropyl-1H-pyrazol-5-yl)ethyl methanesulfonate (5.5 g, 1 eq) and dimethylamine (2 M, 40 mL, 5 eq, THF solution) was stirred at 60° C. for 12 hours. The reaction mixture was diluted with H₂O (80 mL) and extracted with DCM (3×50 mL). The organic phase was washed with brine (2×50 mL) dried over anhydrous Na₂SO₄ filtered and concentrated in vacuum. The residue was purified by silica gel column chromatography (SiO₂, Petroleum ether:Ethyl acetate, 10:1 to 1:1) to give the title compound (3 g, 54.5% yield over two steps) as a yellow solid.

¹H NMR (400 MHz, CDCl₃): δ 6.84 (s, 1H), 6.35 (s, 1H), 3.99-3.94 (m, 1H), 3.57-349 (m, 1H), 2.25 (s, 6H), 1.50 (s, 9H), 1.33 (d, 3H) and 1.02-0.88 (m, 4H).

### Step G: 1-cyclopropyl-5-(1-(dimethylamino)ethyl)-1H-pyrazol-3-amine

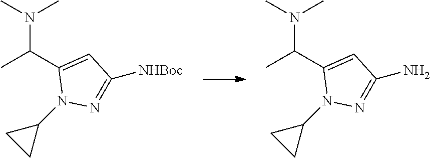 [SMILES: CC(c1cc(NC(=O)OC(C)(C)C)nn1C1CC1)N(C)C ; CC(c1cc(N)nn1C1CC1)N(C)C]

To a solution of tert-butyl (1-cyclopropyl-5-(1-(dimethylamino)ethyl)-1H-pyrazol-3-yl)carbamate (3 g, 10.19 mmol, 1 eq) in DCM (30 mL) was added HCl/EtOAc (4 M, 42.86 mL, 16.82 eq) at 15° C. The mixture was stirred at 15° C. for 0.5 hour. The reaction mixture was concentrated under reduced pressure to give to give the title compound (2.8 g, crude, HCl) as a yellow solid, which was used to the next step directly without further purification.

¹H NMR (400 MHz, DMSO-d₆): δ 6.54 (s, 1H), 4.91-4.85 (m, 1H), 3.82-3.76 (m, 1H), 2.80 (s, 3H), 2.65 (s, 3H), 1.63 (d, 3H) and 1.08-1.04 (m, 4H). Two exchangable protons not observed.

### Step H: 1-(3-bromo-1-cyclopropyl-1H-pyrazol-5-yl)-N,N-dimethylethanamine

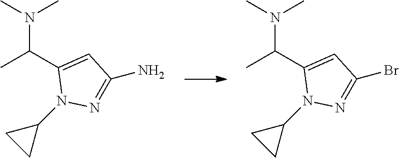

To a solution of 1-cyclopropyl-5-(1-(dimethylamino)ethyl)-1H-pyrazol-3-amine (2.8 g, 12.14 mmol, 1 eq, HCl) and CuBr₂ (1.63 g, 7.28 mmol, 0.6 eq) in MeCN (50 mL) was added CuBr (87 mg, 606.75 μmol, 0.05 eq) and tert-butyl nitrite (1.88 g, 18.20 mmol, 2.16 mL, 1.5 eq) at 0° C. The mixture was stirred at 50° C. for 1 hour. The reaction mixture was quenched with saturated NaHCO₃ solution (100 mL) and extracted with ethyl acetate (2×100 mL). The organic phase was washed with brine (100 mL), dried over anhydrous Na₂SO₄, filtered and concentrated in vacuum. The residue was purified by column chromatography (SiO₂, Petroleum ether:Ethyl acetate, 10:1 to 1:1) to give the title compound (1.5 g, 57.0% yield over two steps, 98% purity on LCMS) as a brown oil.

¹H NMR (400 MHz, CDCl₃): δ 6.12 (s, 1H), 3.96-3.90 (m, 1H), 3.63-3.59 (m, 1H), 2.24 (s, 6H), 1.08 (d, 3H) and 1.07-0.98 (m, 4H).

LCMS: m/z 260 (M+H)⁺ (ES⁺).

### Intermediate L4: 3-bromo-5-(3-methoxyoxetan-3-yl)-1-methyl-1H-pyrazole

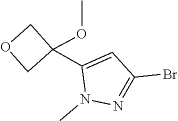

### Step A: tert-butyl (5-(3-hydroxyoxetan-3-yl)-1-methyl-1H-pyrazol-3-yl)carbamate

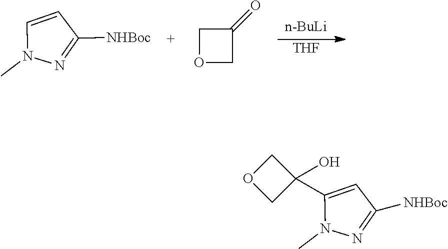

To a solution of tert-butyl (1-methyl-1H-pyrazol-3-yl)carbamate (1 g, 5.07 mmol, 1 eq, obtained according to Step A of the synthesis of intermediate L1) in THF (20 mL) was added with n-BuLi (2.5 M, 4.4 mL, 2.2 eq) at −75° C. The mixture was stirred at −75° C. for 1 hour. Then oxetan-3-one (730 mg, 10.14 mmol, 2 eq) was added into the above mixture at −75° C. After addition, the reaction mixture was warmed to 25° C. and stirred for another 1 hour. The reaction mixture was quenched with water (20 mL) and extracted with EtOAc (3×50 mL). The organic layers were dried over anhydrous Na₂SO₄, filtered and concentrated in vacuum. The residue was triturated with methyl tert-butyl ether (10 mL) to give the title compound (0.4 g, 27.25% yield, 93% purity on LCMS) as a white solid.

¹H NMR (400 MHz, CD₃OD): δ 6.42 (s, 1H), 4.96 (d, 2H), 4.86 (d, 2H), 3.69 (s, 3H) and 1.53 (s, 9H). Two exchangable protons not observed.

LCMS: m/z 270.2 (M+H)⁺ (ES⁺).

### Step B: 3-(3-amino-1-methyl-1H-pyrazol-5-yl)oxetan-3-ol

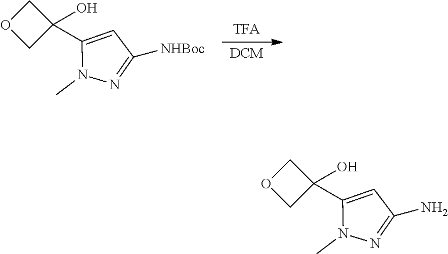 [SMILES: Cn1nc(NC(=O)OC(C)(C)C)cc1C1(O)COC1 ; Cn1nc(N)cc1C1(O)COC1]

To a solution of tert-butyl (5-(3-hydroxyoxetan-3-yl)-1-methyl-H-pyrazol-3-yl)carbamate (0.5 g, 1.86 mmol, 1 eq) in DCM (5 mL) was added TFA (7.7 g, 67.53 mmol, 36.37 eq). The reaction mixture was stirred at 25° C. for 7 hours. The reaction mixture was concentrated in vacuum. The residue was purified by reverse phase flash chromatography (0.1% of NH₃.H₂O in water/MeCN) to give the title compound (0.1 g, 31.84% yield) as a white solid.

¹H NMR (400 MHz, DMSO-d₆): δ 6.35 (s, 1H), 5.49 (s, 1H), 4.74 (d, 2H), 4.68 (d, 2H), 4.48 (s, 2H) and 3.44 (s, 3H).

LCMS: m/z 170.2 (M+H)⁺ (ES⁺).

### Step C: 3-(3-bromo-1-methyl-1H-pyrazol-5-yl)oxetan-3-ol

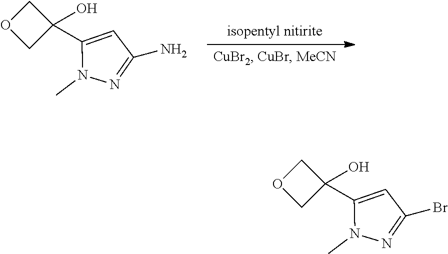

To a solution of 3-(3-amino-1-methyl-1H-pyrazol-5-yl)oxetan-3-ol (2 g, 11.82 mmol, 1 eq) in MeCN (2 mL) was added CuBr₂ (1.58 g, 7.09 mmol, 0.6 eq), CuBr (84 mg, 591.08 μmol, 0.05 eq) and tert-butyl nitrite (1.83 g, 17.73 mmol, 2.11 mL, 1.5 eq). The reaction mixture was stirred at 50° C. for 1 hour. The reaction mixture was concentrated in vacuum. The residue was purified by reverse phase flash chromatography (0.1% NH₃.H₂O/MeCN) to give the title compound (0.8 g, 22.65% yield, 78% purity on LCMS) as a yellow solid.

¹H NMR (400 MHz, CD₃OD): δ 6.46 (s, 1H), 4.94 (d, 2H), 4.83 (s, 2H) and 3.82 (s, 3H). One exchangable proton not observed.

LCMS: m/z 235.0 (M+H)⁺ (ES⁺).

### Step D: 3-bromo-5-(3-methoxyoxetan-3-yl)-1-methyl-1H-pyrazole

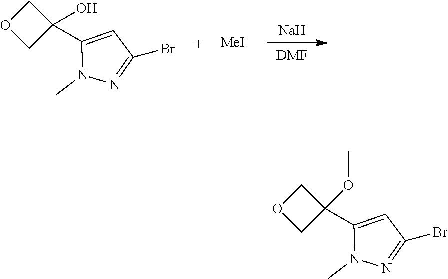

To a mixture of NaH (257 mg, 6.44 mmol, 60 wt. % in mineral oil, 1.5 eq) in DMF (10 mL) was added 3-(3-bromo-1-methyl-1H-pyrazol-5-yl)oxetan-3-ol(1 g, 4.29 mmol, 1 eq) in portions at 0° C. The reaction mixture was stirred at 0° C. for 30 minutes. Then MeI (730 mg, 5.15 mmol, 1.2 eq) was added into the above mixture at 0° C. After addition, the mixture was stirred 25° C. for 1 hour. The reaction mixture was quenched with H₂O (10 mL), extracted with EtOAc (30 mL), washed with saturated aqueous NH₄Cl solution (3×10 mL) and brine (3×1 mL). The organic layer was dried over anhydrous Na₂SO₄, filtered and concentrated in vacuum. The residue was purified by reverse phase flash chromatography (0.1% NH₃.H₂O/MeCN) to give the title compound (450 mg, 41.60% yield, 98% purity on LCMS) as a yellow solid.

¹H NMR (400 MHz, CD₃OD): δ 6.57 (s, 1H), 4.88 (s, 2H), 4.84 (s, 2H), 3.71 (s, 3H) and 3.07 (s, 3H).

LCMS: m/z 247.0 (M+H)⁺ (ES⁺).

### Intermediate L5: 3-bromo-1-isopropyl-5-(3-methoxyoxetan-3-yl)-1H-pyrazole

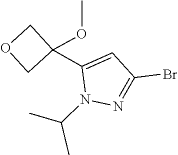

### Step A: tert-butyl (5-(3-hydroxyoxetan-3-yl)-1-isopropyl-1H-pyrazol-3-yl)carbamate

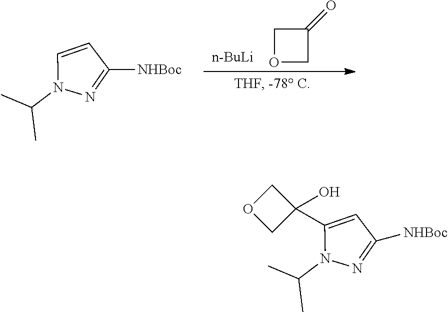

To a solution of tert-butyl (1-isopropyl-1H-pyrazol-3-yl)carbamate (14.8 g, 65.69 mmol, 1 eq, obtained according to Step C of the synthesis of intermediate L2) in THF (300 mL) was added dropwise n-BuLi (2.5 M, 57.81 mL, 2.2 eq) at −75° C. The reaction mixture was stirred at −75° C. for 1 hour. Then oxetan-3-one (14.20 g, 197.08 mmol, 3 eq) was added into the above mixture. The reaction mixture was warmed to 25° C. and stirred at 25° C. for 1 hour. The reaction mixture was quenched with addition of H₂O (500 mL) at 0° C. and extracted with EtOAC (2×500 mL). The organic phases were washed with brine (500 mL), dried over anhydrous Na₂SO₄, filtered and concentrated in vacuum. The residue was purified by silica gel column chromatography (SiO₂, Petroleum ether:Ethyl acetate, 10:1 to 1:1) to give the title compound (16 g, 81.9% yield, 71% purity on LCMS) as a yellow oil.

¹H NMR (400 MHz, CDCl₃): δ 7.09 (s, 1H), 6.43 (s, 1H), 4.99 (d, 2H), 4.84 (d, 2H), 4.25-4.21 (m, 1H), 1.50 (s, 9H) and 1.39 (d, 6H). One exchangable proton not observed.

LCMS: m/z 298.2 (M+H)⁺ (ES⁺).

### Step B: 3-(3-amino-1-isopropyl-1H-pyrazol-5-yl)oxetan-3-ol

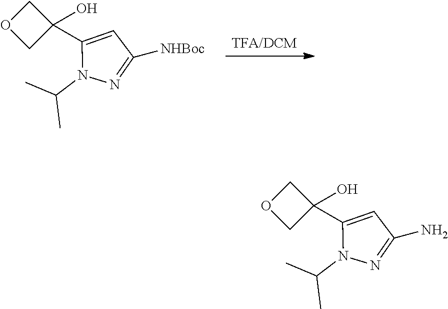 [SMILES: CC(C)n1nc(NC(=O)OC(C)(C)C)cc1C1(O)COC1 ; CC(C)n1nc(N)cc1C1(O)COC1]

To a solution of tert-butyl (5-(3-hydroxyoxetan-3-yl)-1-isopropyl-H-pyrazol-3-yl)carbamate (5 g, 16.82 mmol, 1 eq) in DCM (100 mL) was added TFA (23.10 g, 202.59 mmol, 12.05 eq). The reaction mixture was stirred at 25° C. for 12 hours. Most of the solvent was evaporated under reduced pressure. The residue was purified by reverse phase flash chromatography (0.1% NH₃.H₂O/MeCN) to give the title compound (1.5 g, 42.51% yield, 94% purity on LCMS) as a yellow solid.

¹H NMR (400 MHz, DMSO-d₆): δ 6.35 (s, 1H), 5.44 (s, 1H), 4.77-4.61 (m, 6H), 4.14-4.04 (m, 1H) and 1.29-1.22 (m, 6H).

LCMS: m/z 198.2 (M+H)⁺ (ES⁺).

### Step C: 3-(3-bromo-1-isopropyl-1H-pyrazol-5-yl)oxetan-3-ol

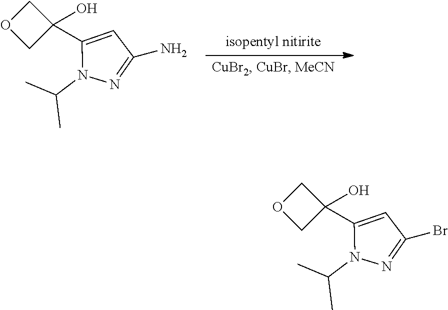

To a solution of 3-(3-amino-1-isopropyl-1H-pyrazol-5-yl)oxetan-3-ol(1.5 g, 4.82 mmol, 1 eq) in MeCN (8 mL) was added CuBr (34 mg, 240.96 μmol, 0.05 eq) and CuBr₂ (646 mg, 2.89 mmol, 0.6 eq). Then tert-butyl nitrite (745 mg, 7.23 mmol, 1.5 eq) was added dropwise into the above mixture. The reaction mixture was stirred at 50° C. for 1 hour. The reaction mixture was purified directly by reverse phase flash chromatography (0.1% of NH₃.H₂/MeCN) to give the title compound (1 g, 58.81% yield, 74% purity on LCMS) as a brown oil.

¹H NMR (400 MHz, CDCl₃): δ 6.21 (s, 1H), 5.01-4.87 (m, 4H), 3.75-3.69 (m, 1H) and 1.47 (d, 6H). One exchangable proton not observed.

LCMS: m/z 263.0 (M+H)⁺ (ES⁺).

### Step D: 3-bromo-1-isopropyl-5-(3-methoxyoxetan-3-yl)-1H-pyrazole

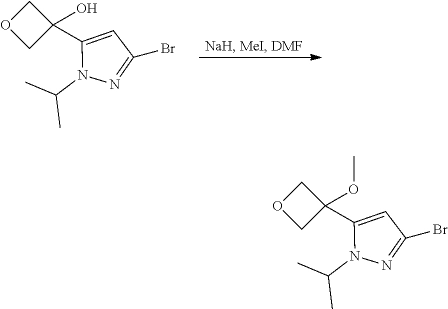

A solution of 3-(3-bromo-1-isopropyl-1H-pyrazol-5-yl)oxetan-3-ol(0.8 g, 3.06 mmol, 1 eq) in DMF (10 mL) was cooled to 0° C. Then NaH (147 mg, 3.68 mmol, 60 wt. % in mineral oil, 1.2 eq) was added and the resulting mixture stirred at 0° C. for 0.5 hour. MeI (652 mg, 4.60 mmol, 1.5 eq) was added dropwise into the mixture. The mixture was stirred at 25° C. for 2 hours. The reaction mixture was diluted with H₂O (50 mL) and extracted with EtOAc (3×50 mL). The combined organic layers were washed with brine (2×20 mL), dried over Na₂SO₄, filtered and concentrated under reduced pressure. The residue was purified by reverse phase flash chromatography (0.1% of NH₃.H₂O/MeCN) to give the title compound (300 mg, 28.83% yield, 81% purity on LCMS) as a yellow solid.

¹H NMR (400 MHz, DMSO-d₆): δ 6.67 (s, 1H), 4.84-4.76 (m, 4H), 4.18-4.11 (m, 1H), 3.00 (s, 3H) and 1.32 (d, 6H).

### Intermediate L6: 3-bromo-1-cyclopropyl-5-(3-methoxyoxetan-3-yl)-1H-pyrazole

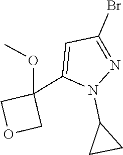

### Step A: tert-butyl (1-cyclopropyl-5-(3-hydroxyoxetan-3-yl)-1H-pyrazol-3-yl)carbamate

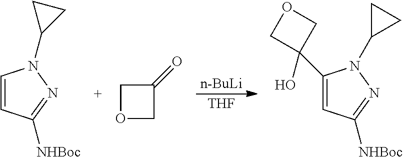

To a solution of tert-butyl (1-cyclopropyl-1H-pyrazol-3-yl)carbamate (15.5 g, 69.42 mmol, 1 eq, obtained according to Step C of the synthesis of intermediate L3) in THF (200 mL) was added dropwise n-BuLi (2.5 M, 61.09 mL, 2.2 eq) at −70° C. The reaction mixture was stirred at −70° C. for 0.5 hour. Then oxetan-3-one (10.01 g, 138.84 mmol, 2 eq) was added dropwise into the above mixture at −70° C. The reaction mixture was warmed to 25° C. and stirred at 25° C. for 0.5 hours. The reaction mixture was quenched by addition of saturated aqueous NH₄Cl solution (70 mL) at 25° C. The mixture was diluted with H₂O (200 mL) and extracted with EtOAc (2×100 mL). The combined organic layers were washed with brine (2×100 mL), dried over Na₂SO₄, filtered and concentrated under reduced pressure. The residue was purified by silica gel column chromatography (SiO₂, Petroleum ether:Ethyl acetate, 30:1 to 0:1) to give the title compound (15.2 g, 74.14% yield) as a white solid.

¹H NMR (400 MHz, DMSO-d₆): δ 6.50 (s, 1H), 6.33 (br s, 1H), 4.89 (d, 2H), 4.73 (d, 2H), 3.54-3.30 (m, 1H), 1.43 (s, 9H), 1.03-0.98 (m, 2H) and 0.86-0.81 (m, 2H). One exchangable proton not observed.

LCMS: m/z 318.2 (M+Na)⁺ (ES⁺).

### Step B: 3-(3-amino-1-cyclopropyl-1H-pyrazol-5-yl)oxetan-3-ol

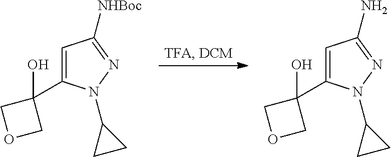

To a mixture of tert-butyl (1-cyclopropyl-5-(3-hydroxyoxetan-3-yl)-1H-pyrazol-3-yl)carbamate (5 g, 16.93 mmol, 1 eq) in DCM (50 mL) was added TFA (30.80 g, 270.12 mmol, 15.96 eq). The reaction mixture was stirred at 25° C. for 5 hours. The reaction mixture was concentrated in vacuum. The residue was re-dissolved in MeOH (30 mL) and the mixture was adjusted with NH₃.H₂O (25%) to pH 9-10. Most of solid precipitated out. Then the mixture was filtered and the filter cake was dried under vacuum to give the title compound (2.7 g, 81.69% yield) as a white solid.

¹H NMR (400 MHz, CDCl₃): δ 5.60 (s, 1H), 5.03 (d, 2H), 4.88 (d, 2H), 3.37-3.31 (m, 1H), 1.17-1.15 (m, 2H) and 0.92-0.88 (m, 2H). Three exchangable protons not observed.

LCMS: m/z 196.1 (M+H)⁺ (ES⁺).

### Step C: 3-(3-bromo-1-cyclopropyl-1H-pyrazol-5-yl)oxetan-3-ol

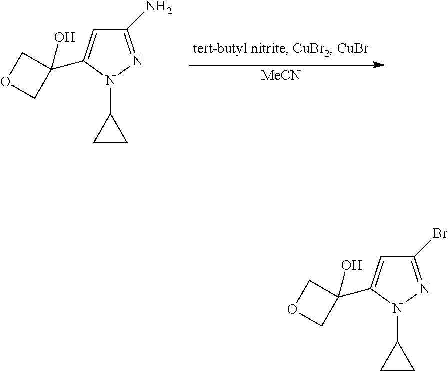 [SMILES: "MeCN" ; Nc1cc(C2(O)COC2)n(C2CC2)n1 ; OC1(c2cc(Br)nn2C2CC2)COC1]

To a solution of 3-(3-amino-1-cyclopropyl-1H-pyrazol-5-yl)oxetan-3-ol(2.7 g, 13.83 mmol, 1 eq) in MeCN (30 mL) was added CuBr (99 mg, 691.53 μmol, 0.05 eq) and CuBr₂ (1.54 g, 6.92 mmol, 0.5 eq). Then tert-butyl nitrite (2.14 g, 20.75 mmol, 2.47 mL, 1.5 eq) was added dropwise to the above mixture at 25° C. The reaction mixture was stirred at 25° C. for 1 hour. The reaction mixture was diluted with H₂O (50 mL) and NH₃.H₂O (25%, 20 mL). The mixture was extracted with EtOAc (2×50 mL), washed with brine, dried over Na₂SO₄, filtered and concentrated under vacuum. The residue was purified by reverse phase flash chromatography (0.05% of NH₃.H₂O/CH₃CN) to give the title compound (1.4 g, 39.07% yield) as a yellow oil.

¹H NMR (400 MHz, CDCl₃): δ 6.26 (s, 1H), 5.02 (d, 2H), 4.90 (d, 2H), 3.57-3.51 (m, 1H), 1.27-1.25 (m, 2H) and 1.00-0.94 (m, 2H). One exchangable proton not observed.

LCMS: m/z 259.0 (M+H)⁺ (ES⁺).

### Step D: 3-bromo-1-cyclopropyl-5-(3-methoxyoxetan-3-yl)-1H-pyrazole

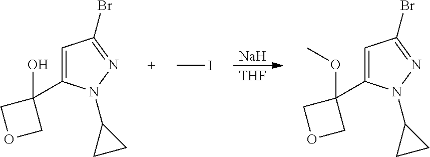

To a solution of 3-(3-bromo-1-cyclopropyl-1H-pyrazol-5-yl)oxetan-3-ol(1.2 g, 4.63 mmol, 1 eq) in THF (20 mL) was added NaH (278 mg, 6.95 mmol, 60 wt. % in mineral oil, 1.5 eq) at 0° C. The reaction mixture was stirred at 0° C. for 0.5 hour. Then MeI (4.5 g, 31.70 mmol, 6.85 eq) was added into the above mixture at 0° C. The reaction mixture was warmed to 20° C. and stirred at 20° C. for 0.5 hour. The reaction mixture was quenched by addition of H₂O (40 mL) at 20° C. and extracted with EtOAc (2×30 mL). The combined organic layers were washed with brine (2×20 mL), dried over Na₂SO₄, filtered and concentrated under reduced pressure. The residue was purified by silica gel column chromatography (SiO₂, Petroleum ether:Ethyl acetate, 50:1 to 10:1) to give the title compound (0.99 g, 78.26% yield) as a yellow oil.

¹H NMR (400 MHz, DMSO-d₆): δ 6.73 (s, 1H), 4.88 (d, 2H), 4.79-4.75 (m, 2H), 3.48-3.41 (m, 1H), 3.01 (s, 3H), 1.08-1.05 (m, 2H) and 0.92-0.87 (m, 2H).

LCMS: m/z 275.0 (M+H)⁺ (ES⁺).

### Intermediate L7: 3-bromo-1-(2-methoxy-2-methylpropyl)-1H-pyrazole

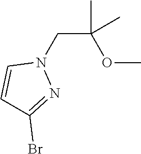

### Step A: 2-methyl-1-(3-nitro-1H-pyrazol-1-yl)propan-2-ol

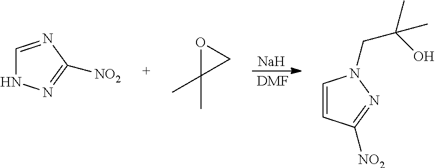

To a mixture of 3-nitro-1H-pyrazole (5 g, 44.22 mmol, 1 eq) in DMF (50 mL) was added NaH (2.12 g, 53.06 mmol, 60 wt. % in mineral oil, 1.2 eq) at 0° C. The reaction mixture was stirred at 0° C. for 0.5 hour. Then 2,2-dimethyloxirane (3.51 g, 48.64 mmol, 1.1 eq) was added into the above mixture at 0° C. The resulting mixture was warmed to 25° C. and stirred at 25° C. for 1 hour. The reaction mixture was quenched by addition of H₂O (100 mL) and extracted with EtOAc (2×50 mL). The combined organic layers were washed with brine (2×30 mL), dried over Na₂SO₄, filtered and concentrated under reduced pressure to give the title compound (3.7 g, crude) as yellow oil, which was used directly in the next step.

¹H NMR (400 MHz, CDCl₃): δ 7.60 (dd, 1H), 6.93 (d, 1H), 4.21 (s, 2H) and 1.27 (s, 6H). One exchangable proton not observed.

LCMS: m/z 208.1 (M+Na)⁺ (ES⁺).

### Step B: 1-(2-methoxy-2-methylpropyl)-3-nitro-1H-pyrazole

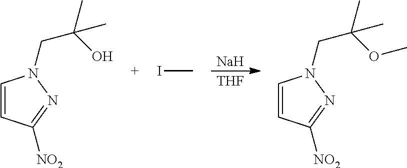

To a mixture of 2-methyl-1-(3-nitro-1H-pyrazol-1-yl)propan-2-ol(3.7 g, 19.98 mmol, 1 eq) in THF (60 mL) was added NaH (879 mg, 21.98 mmol, 60 wt. % in mineral oil, 1.1 eq) at 0° C. The reaction mixture was stirred at 0° C. for 0.5 hour. Then MeI (4.25 g, 29.97 mmol, 1.5 eq) was added into the above mixture in portions at 0° C. The reaction mixture was warmed to 25° C. and stirred at 25° C. for 1 hour. The reaction mixture was quenched by addition of H₂O (30 mL) at 25° C. and extracted with EtOAc (2×30 mL). The combined organic layers were washed with brine (2×20 mL), dried over Na₂SO₄, filtered and concentrated under reduced pressure. The residue was purified by silica gel column chromatography (SiO₂, Petroleum ether:Ethyl acetate, 50:1 to 5:1) to give the title compound (1.6 g, 18.2% yield over two steps) as a yellow oil.

¹H NMR (400 MHz, CDCl₃): δ 7.58 (d, 1H), 6.89 (d, 1H), 4.19 (s, 2H), 3.24 (s, 3H) and 1.16 (s, 6H).

### Step C: 1-(2-methoxy-2-methylpropyl)-1H-pyrazol-3-amine

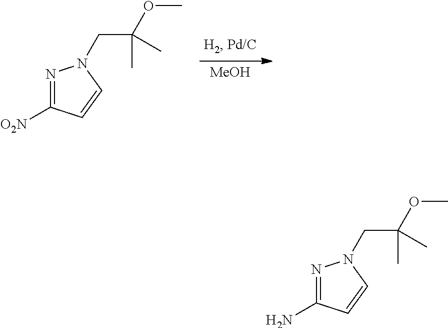

To a solution of 1-(2-methoxy-2-methylpropyl)-3-nitro-1H-pyrazole (1.6 g, 8.03 mmol, 1 eq) in MeOH (20 mL) was added Pd/C (0.5 g, 10 wt. % loading on activated carbon) under N₂. The suspension was degassed in vacuum and purged with H₂ several times. The reaction mixture was stirred at 30° C. for 12 hours under H₂ (40 psi). The reaction mixture was filtered and the filtrate was concentrated in vacuum to give the title compound (1.34 g, 98.59% yield) as yellow oil.

¹H NMR (400 MHz, CDCl₃): δ 7.20 (d, 1H), 5.60 (d, 1H), 3.90 (s, 2H), 3.62 (s, 2H), 3.24 (s, 3H) and 1.15 (s, 6H).

LCMS: m/z 170.2 (M+H)⁺ (ES⁺).

### Step D: 3-bromo-1-(2-methoxy-2-methylpropyl)-1H-pyrazole

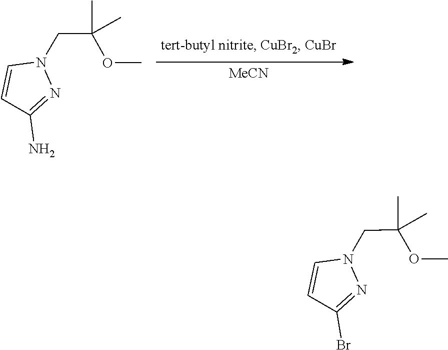

To a mixture of 1-(2-methoxy-2-methylpropyl)-1H-pyrazol-3-amine (1.14 g, 6.74 mmol, 1 eq) in MeCN (20 mL) was added tert-butyl nitrite (1.04 g, 10.10 mmol, 1.5 eq), CuBr₂ (1.05 g, 4.72 mmol, 0.7 eq) and CuBr (48 mg, 336.83 μmol, 0.05 eq). The reaction mixture was stirred at 70° C. for 2 hours. The reaction mixture was diluted with H₂O (50 mL) and extracted with EtOAc (2×50 mL). Then the organic layers were washed with brine (2×50 mL), dried over Na₂SO₄, filtered and concentrated in vacuum. The residue was purified by reverse phase flash chromatography (0.05% of NH₃.H₂O/MeCN) to give the title compound (0.55 g, 35.02% yield) as a yellow oil.

¹H NMR (400 MHz, CDCl₃): δ 7.39 (d, 1H), 6.26 (d, 1H), 4.08 (s, 2H), 3.23 (s, 3H) and 1.14 (s, 6H).

LCMS: m/z 257.0 (M+Na)⁺ (ES⁺).

### Intermediate L8: 3-iodo-1-isopropyl-1H-pyrazole

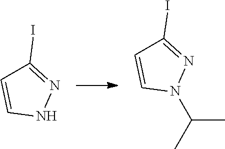

To a solution of 3-iodo-1H-pyrazole (10 g, 51.55 mmol, 1 eq) in DMF (100 mL) was added NaHMDS (1 M, 61.86 mL, 1.2 eq) at 0° C. The reaction mixture was stirred at 0° C. for 0.5 hours. Then a solution of 2-iodopropane (10.52 g, 61.86 mmol, 1.2 eq) in DMF (20 mL) was added dropwise to the above mixture. The reaction mixture was warmed to 25° C. and stirred for 12 hours. The reaction mixture was quenched with water (100 mL) and extracted EtOAc (3×80 mL). The organic layers were dried over anhydrous Na₂SO₄ filtered and concentrated in vacuum. The residue was purified by column chromatography (SiO₂, petroleum ether:ethyl acetate 1:0 to 50:1) to give the title compound (6.9 g, 56% yield) as a yellow oil.

¹H NMR (400 MHz, CDCl₃): δ 7.26 (d, 1H), 6.40 (d, 1H), 4.56-4.48 (m, 1H) and 1.50 (d, 6H).

### Intermediate L9: 2-(3-iodophenyl)propan-2-ol

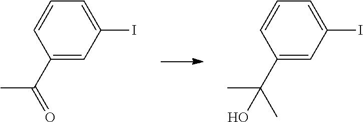

To a solution of 1-(3-iodophenyl) ethanone (1 g, 4.06 mmol, 1 eq) in THF (10 mL) was added MeMgBr (3 M, 2.71 mL, 2 eq) at 0° C. After addition, the mixture was stirred at 0° C. for 2 hours. The reaction mixture was quenched with saturated aqueous NH₄C solution (10 mL) and extracted with ethyl acetate (3×10 mL). The organic layers were dried over anhydrous Na₂SO₄, filtered and concentrated in vacuum. The residue was purified by prep-HPLC (column: Waters Xbridge C18, 150 mm*50 mm*10 μm; mobile phase: [A: water (0.05% ammonia hydroxide v/v), B: MeCN]; B %: 32%-62%,11.5 minutes) to give the title compound (100 mg, 9.3% yield) as a white solid.

¹H NMR (400 MHz, CDCl₃) δ 7.87 (s, 1H), 7.59 (d, 1H), 7.45 (d, 1H), 7.08 (t, 1H), 1.57 (s, 6H). One exchangeable proton not observed.

### Intermediate L10: 2-(4-iodophenyl)propan-2-ol

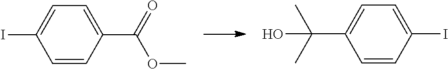

To a solution of methyl 4-iodobenzoate (1 g, 3.82 mmol, 1 eq) in THF (10 mL) was added MeMgBr (3 M, 5.09 mL, 4 eq). After addition, the mixture was stirred at ° C. for 2 hours. The reaction mixture was quenched with saturated aqueous NH₄Cl solution (10 mL) and extracted with ethyl acetate (3×1 mL). The organic layers were dried over anhydrous Na₂SO₄, filtered and concentrated in vacuum. The residue was purified by silica gel column chromatography (SiO₂, petroleum ether:ethyl acetate, 10:1 to 2:1) to give the title compound (600 mg, 56.3% yield, 94% purity on LCMS) as yellow oil.

¹H NMR (400 MHz, CDCl₃) δ 7.70 (d, 2H), 7.30 (d, 2H), 1.60 (s, 6H). One exchangeable proton not observed.

LCMS: m/z 261.0 (M−H)⁻ (ES⁺)

### Intermediate L11: 6-iodo-2-methylisoindolin-1-one

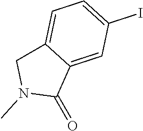

### Step A: 6-iodoisoindolin-1-one

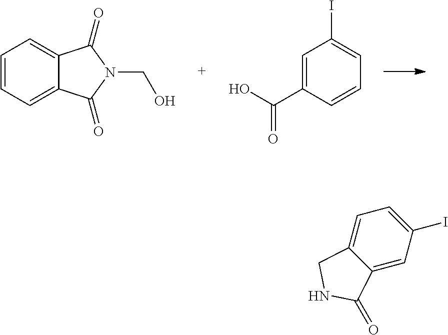 [SMILES: O=C1c2ccccc2C(=O)N1CO ; O=C(O)c1cccc(I)c1 ; O=C1NCc2ccc(I)cc21]

To a solution of 3-iodobenzoic acid (1 g, 4.03 mmol, 1 eq) in concentrated H₂SO₄ (10 mL) was added 2-(hydroxymethyl)isoindoline-1,3-dione (857 mg, 4.84 mmol, 1.2 eq). The reaction mixture was stirred at 90° C. for 3 hours. The mixture was added into H₂O (100 mL) and filtered. The filter cake was added into NH₃.H₂O (15 mL, 25 wt. % in H₂O) and MeOH (15 mL), and then the mixture was stirred at 80° C. for 1 hour. The mixture was filtered, and the filter cake was dried in vacuum to give the title compound (600 mg, 57.4% yield, 94.0% purity on LCMS) as a white solid.

¹H NMR (400 MHz, DMSO-d₆) δ 8.66 (s, 1H), 7.95-7.95 (m, 2H), 7.41 (d, 1H), 4.37 (s, 2H).

LCMS: m/z 259.9 (M+H)⁺ (ES⁺).

### Step B: 6-iodo-2-methylisoindolin-1-one

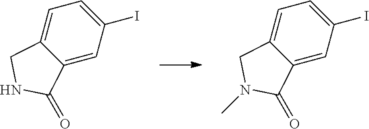

To a solution of 6-iodoisoindolin-1-one (500 mg, 1.93 mmol, 1 eq) in DMF (5 mL) at 0° C. was added NaH (115 mg, 2.90 mmol, 60 wt. % in mineral oil, 1.5 eq). The mixture was stirred at ° C. for 30 minutes, then MeI (410 mg, 2.90 mmol, 1.5 eq) was added dropwise. The mixture was warmed to 20° C. and stirred for 3 hours. The mixture was diluted with H₂O (15 mL) and extracted with EtOAc (3×20 mL). The organic phases were dried over anhydrous Na₂SO₄, filtered and concentrated in vacuum. The residue was purified by prep-HPLC (column: Xtimate C18, 150 mm*25 mm*5 μm; mobile phase: [A: water (0.05% ammoniahydroxide v/v), B: MeCN]; B %: 20%-50%,10 min) to give the title compound (300 mg, 56.9% yield, 100% purity on LCMS) as a white solid.

¹H NMR (400 MHz, CDCl₃) δ 8.18 (s, 1H), 7.84 (dd, 1H), 7.20 (d, 1H), 4.32 (s, 2H), 3.20 (s, 3H).

LCMS: m/z 274.0 (M+H)⁺ (ES⁺).

### Intermediate R1: 5-((1,2,3,5,6,7-hexahydro-s-indacen-4-yl)amino)-4H-1,2,4-triazole-3-thiol

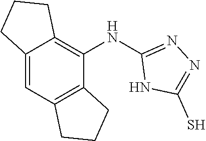 [SMILES: Sc1nnc(Nc2c3c(cc4c2CCC4)CCC3)[nH]1]

### Step A: 4-isothiocyanato-1,2,3,5,6,7-hexahydro-s-indacene

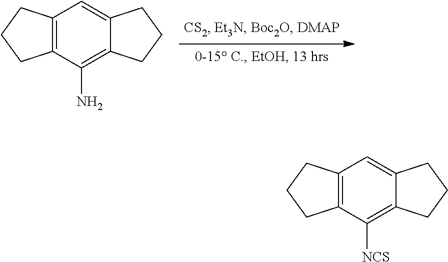

To a solution of 1,2,3,5,6,7-hexahydro-s-indacen-4-amine (60 g, 346.31 mmol, 1 eq) in EtOH (900 mL) was added CS₂ (79.11 g, 1.04 mol, 3 eq) and TEA (35.04 g, 346.31 mmol, 1 eq). The reaction mixture was stirred at 15° C. for 1 hour. The reaction mixture was cooled down to 0° C. Then Boc₂O (75.58 g, 346-31 mmol, 79.56 mL, 1 eq) and DMAP (1.27 g, 10-39 mmol, 0.03 eq) were added into the above reaction mixture. The resulting mixture was stirred for 15° C. for 12 hours. The reaction mixture was concentrated in vacuum. The residue was treated with PE (1.5 L) and the mixture was stirred for 1 hour. The mixture was filtered and the filtrate was concentrated in vacuum to give the title compound (45 g, 60.35% yield) as a yellow solid.

¹H NMR (400 MHz, CDCl₃): δ 7.00 (s, 1H), 2.96-2.87 (m, 8H) and 2.15-2.08 (m, 4H).

### Step B: 1-carbamothioyl-3-(1,2,3,5,6,7-hexahydro-s-indacen-4-yl)thiourea

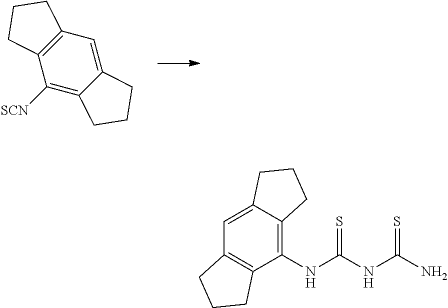

To a solution of thiourea (15.56 g, 204.35 mmol, 1 eq) in MeCN (800 mL) and H₂O (80 mL) was added NaOH (8.17 g, 204.35 mmol, 1 eq). The resulting mixture was heated to 40° C. and stirred for 20 minutes. Then the mixture was cooled down to 16° C. 4-isothiocyanato-1,2,3,5,6,7-hexahydro-s-indacene (44 g, 204.35 mmol, 1 eq) was added into the above mixture. The mixture was stirred at 35° C. for 12 hours. H₂O (400 mL) was added into the reaction mixture and the mixture was adjusted to pH 2 with concentrated HCl. The mixture was concentrated in vacuum to remove most of the MeCN and the solid precipitated out. The mixture was filtered and the solid was dried to give the title compound (30 g, 50.37% yield) as a yellow solid.

¹H NMR (400 MHz, DMSO-d₆): δ12.09 (s, 1H), 10.83 (s, 1H), 9.25-9.23 (m, 2H), 7.05 (s, 1H), 2.87-2.68 (m, 8H) and 2.04-1.98 (m, 4H).

LCMS: m/z 292.0 (M+H)⁺ (ES⁺).

### Step C: ethyl N′-(1,2,3,5,6,7-hexahydro-s-indacen-4-yl)carbamothioyl-carbamimidothioate and ethyl N′-carbamothioyl-N-(1,2,3,5,6,7-hexahydro-s-indacen-4-yl)carbamimidothioate

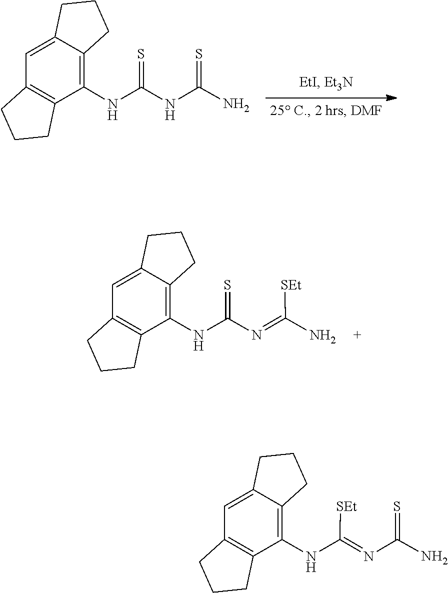

To a solution of 1-carbamothioyl-3-(1,2,3,5,6,7-hexahydro-s-indacen-4-yl)thiourea (30 g, 102.94 mmol, 1 eq) in DMF (300 mL) was added TEA (10.42 g, 102.94 mmol, 1 eq) and EtI (14.61 g, 93.68 mmol, 0.91 eq). The mixture was stirred at 25° C. for 2 hours. The reaction mixture was poured into water (500 mL) and extracted with EtOAc (3×300 mL). The combined organic layers were washed with brine (5×150 mL), dried over Na₂SO₄, filtered and concentrated in vacuum. The residue was triturated with EtOH (100 mL) to give a mixture of products (17.8 g, 53.78% yield) as a white solid.

LCMS: m/z 320 (M+H)⁺ (ES⁺).

### Step D: 5-((1,2,3,5,6,7-hexahydro-s-indacen-4-yl)amino)-4H-1,2,4-triazole-3-thiol

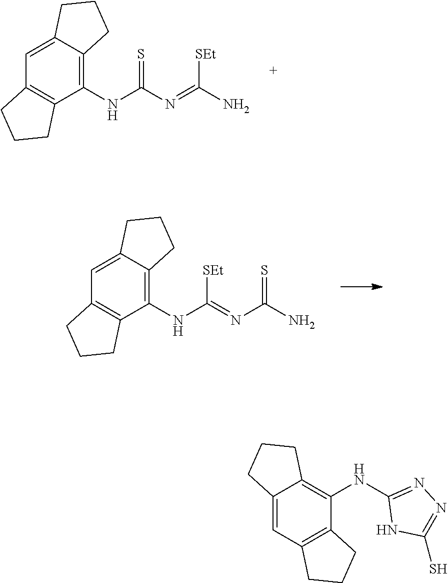

To a solution of ethyl N′-carbamothioyl-N-(1,2,3,5,6,7-hexahydro-s-indacen-4-yl)carbamimidothioate and ethyl N′-(1,2,3,5,6,7-hexahydro-s-indacen-4-yl)carbamothioylcarbamimidothioate (17.80 g, 55.36 mmol, mixture, 1 eq) in EtOH (155 mL) and AcOH (15 mL) was added NH₂NH₂ (56.57 g, 1.66 mol, 45 mL, 30 eq). The reaction mixture was stirred at 80° C. for 2 hours. The reaction mixture was diluted with water (100 mL) and adjusted to pH 2 with concentrated HCl. Some solid precipitated out and the mixture was filtered. The filter cake was collected and triturated with MeOH (300 mL) to give the title compound (2.8 g, 17.25% yield, 92.9% purity on LCMS) as a white solid.

¹H NMR (400 MHz, DMSO-d₆): δ 12.55 (s, 1H), 12.37 (s, 1H), 8.01 (s, 1H), 6.88 (s, 1H), 2.80 (t, 4H), 2.63 (t, 4H) and 2.00-1.92 (m, 4H).

LCMS: m/z 273 (M+H)⁺ (ES⁺).

### Intermediate R2: 5-(4-fluoro-2,6-diisopropylbenzyl)-4H-1,2,4-triazole-3-thiol

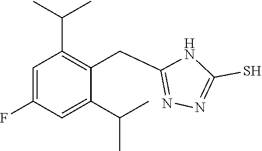 [SMILES: CC(C)c1cc(F)cc(C(C)C)c1Cc1nnc(S)[nH]1]

### Step A: 4-fluoro-2,6-di(prop-1-en-2-yl)aniline

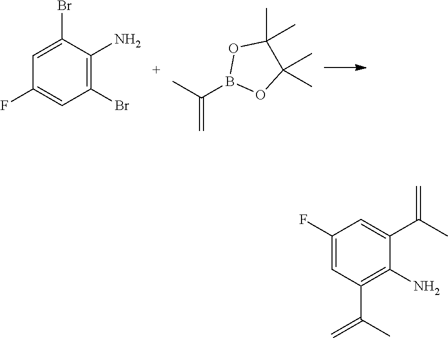

A solution of 2,6-dibromo-4-fluoroaniline (10 g, 1 eq), 4,4,5,5-tetramethyl-2-(prop-1-en-2-yl)-1,3,2-dioxaborolane (16.67 g, 2.67 eq), Cs₂CO₃ (36.35 g, 3 eq) and Pd(dppf)Cl₂ (2.72 g, 3.72 mmol, 0.1 eq) in dioxane (100 mL) and H₂O (10 mL) was degassed under reduced pressure. The mixture was then heated to 100° C. for 3 hours under N₂. The reaction mixture was quenched by addition of H₂O (200 mL), and diluted with EtOAc (150 mL). The mixture was extracted with EtOAc (2×150 mL). The combined organic layers were washed with brine (2×200 mL), dried over anhydrous Na₂SO₄, filtered and concentrated under reduced pressure. The residue was purified by column chromatography (SiO₂, petroleum ether:ethyl acetate 1:0 to 100:1) to give the title compound (8 g, 88.8% yield, 78.9% purity on LCMS) as a yellow oil.

¹H NMR (400 MHz, CDCl₃): δ 6.68 (d, 2H), 5.32-5.31 (m, 2H), 5.08 (d, 2H), 3.84 (s, 2H) and 2.07 (d, 6H).

LCMS: m/z 192.2 (M+H)⁺ (ES⁺).

### Step B: 4-fluoro-2,6-diisopropylaniline

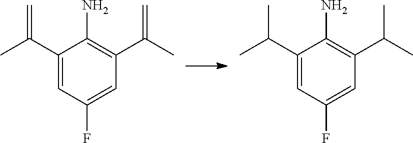

To a solution of 4-fluoro-2,6-di(prop-1-en-2-yl)aniline (8 g, 1 eq) in MeOH (150 mL) was added Pd/C (624 mg, 10 wt. % loading on activated carbon). Then the mixture was degassed and purged with H₂ (20 psi). The reaction mixture was stirred at 25° C. for 12 hours under H₂ (20 psi). The reaction mixture was filtered. The filtrate was concentrated in vacuum. The residue was purified by column chromatography (SiO₂, only eluting with petroleum ether) to give the title compound (4 g, 62.78% yield, 100% purity on LCMS) as a colourless oil.

¹H NMR (400 MHz, CDCl₃): δ 6.76 (d, 2H), 3.56 (s, 2H), 2.99-2.89 (m, 2H) and 1.26 (d, 12H).

LCMS: m/z 196.2 (M+H)⁺ (ES⁺).

### Step C: 2-bromo-5-fluoro-1,3-diisopropylbenzene

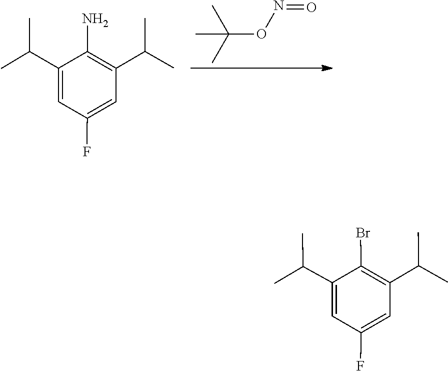

To a solution of 4-fluoro-2,6-diisopropylaniline (3.7 g, 18-95 mmol, 1 eq) in MeCN (180 mL) was added CuBr (4.08 g, 1.5 eq), then tert-butyl nitrite (2.93 g, 1.5 eq) was added dropwise to the reaction mixture at 0° C. The resulting mixture was stirred at 60° C. for 1.5 hours. The reaction mixture was concentrated in vacuum. The residue was purified by column chromatography (SiO₂, only eluting with petroleum ether) to give the title compound (2.02 g, 41.14% yield) as a white solid.

¹H NMR (400 MHz, CDCl₃): δ 6.85 (d, 2H), 3.55-3.48 (m, 2H) and 1.24 (d, 12H).

### Step D: (2-(tert-butoxy)-2-oxoethyl) zinc (II) bromide

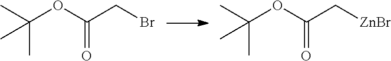

A mixture of zinc (80 g) in HCl (1 M, 308 mL) was stirred at 25° C. for 30 minutes. Then the mixture was filtered and the filter cake was dried in vacuum. To a suspension of the pretreated Zn (55 g, 841.11 mmol, 2.98 eq) in THF (550 mL) was added TMSCl (3.06 g, 28.20 mmol, 0.1 eq) and 1,2-dibromoethane (5.30 g, 28.20 mmol, 0.1 eq) at 20° C. under N₂. Then tert-butyl 2-bromoacetate (55 g, 281.97 mmol, 1 eq) was added into the mixture at 50° C. under N₂. The mixture was stirred at 50° C. for 2 hours. The mixture (theoretical amount: 0.5 M, 550 mL, in THF solution) was cooled and used into the next step without further purification.

### Step E: tert-butyl 2-(4-fluoro-2,6-diisopropylphenyl)acetate

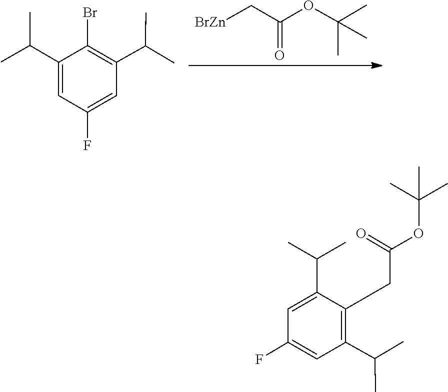

A solution of 2-bromo-5-fluoro-1,3-diisopropylbenzene (16 g, 61.74 mmol, 1 eq) in THF (100 mL) was cooled to 0° C. Then Pd₂(dba)₃ (2.83 g, 3.09 mmol, 0.05 eq), XPhos (2.94 g, 6.17 mmol, 0.1 eq) and (2-(tert-butoxy)-2-oxoethyl)zinc(II) bromide (0.5 M, 246.95 mL, in THF solution, 2 eq) were added into the mixture. The reaction mixture was stirred at 70° C. for 12 hours. The reaction mixture was concentrated in vacuum. The residue was purified by column chromatography (SiO₂, petroleum ether:ethyl acetate 100:0 to 10:1) to give the title compound (12 g, 59.42% yield, 90% purity on ¹H NMR) as a red oil.

¹H NMR (400 MHz, CDCl₃): δ 6.83 (d, 2H), 3.66 (s, 2H), 3.21-3.14 (m, 2H), 1.43 (s, 9H) and 1.21 (d, 12H).

### Step F: 2-(4-fluoro-2,6-diisopropylphenyl)acetic acid

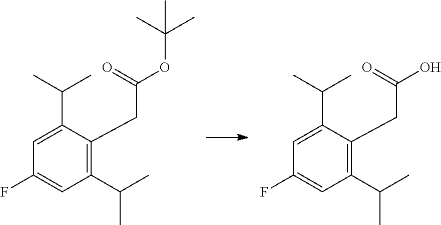 [SMILES: CC(C)c1cc(F)cc(C(C)C)c1CC(=O)OC(C)(C)C ; CC(C)c1cc(F)cc(C(C)C)c1CC(=O)O]

To a solution of tert-butyl 2-(4-fluoro-2,6-diisopropylphenyl)acetate (12 g, 40.76 mmol, 1 eq) in DCM (120 mL) was added TFA (184.80 g, 39.76 eq). The reaction mixture was stirred at 25° C. for 3 hours. Most of the solvents were evaporated under reduced pressure. The residue was diluted with H₂O (300 mL) and the mixture was adjusted to pH 10 with 2 M aqueous NaOH solution. The mixture was washed with EtOAc (3×500 mL) and the organic phases were discarded. Then the aqueous layer was adjusted to pH 3 with 1M aqueous HCl solution and extracted with EtOAc (3×500 mL). The combined organic layers were washed with brine (2×200 mL), dried over Na₂SO₄, filtered and concentrated under reduced pressure to give the title compound (8 g, 82.36% yield) as a yellow solid.

¹H NMR (400 MHz, DMSO-d₆): δ 12.24 (br s, 1H), 6.91 (d, 2H), 3.78 (s, 2H), 3.16-3.06 (m, 2H) and 1.18 (d, 12H).

### Step G: 2-(4-fluoro-2,6-diisopropylphenyl)acetyl chloride

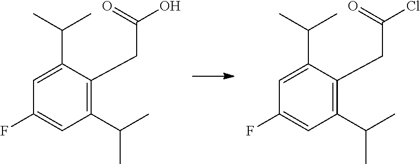 [SMILES: CC(C)c1cc(F)cc(C(C)C)c1CC(=O)O ; CC(C)c1cc(F)cc(C(C)C)c1CC(=O)Cl]

A solution of 2-(4-fluoro-2,6-diisopropyl-phenyl)acetic acid (1 g, 4.20 mmol, 1 eq) in SOCl₂ (15 mL) was stirred at 50° C. for 1 hour. The reaction mixture was concentrated in vacuum to give the title compound (1 g, crude) as a yellow oil, which was used to the next step directly without further purification.

### Step H: 2-(2-(4-fluoro-2,6-diisopropylphenyl)acetyl)hydrazinecarbothioamide

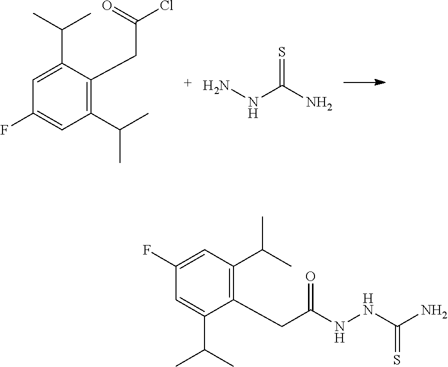

A mixture of hydrazinecarbothioamide (0.35 g, 3.84 mmol, 1 eq) in pyridine (4.25 g, 53.77 mmol, 14 eq) was cooled to 0° C., then a solution of 2-(4-fluoro-2,6-diisopropylphenyl)acetyl chloride (1.04 g, 4.03 mmol, 1.05 eq) in toluene (10 mL) was added dropwise into the above mixture. The reaction mixture was warmed to 25° C. and stirred for 2 hours. The reaction mixture was concentrated in vacuum. The residue was purified by column chromatography (SiO₂, petroleum ether:ethyl acetate 10:1 to 0:1) to give the title compound (350 mg, 26.8% yield over two steps, 79% purity on LCMS) as a yellow solid.

¹H NMR (400 MHz, DMSO-d₆): δ 9.91 (br s, 1H), 9.29 (br s, 1H), 7.90 (br s, 1H), 7.53 (br s, 1H), 6.89 (s, 1H), 6.86 (s, 1H), 3.66 (s, 2H), 3.18-3.09 (m, 2H), and 1.18 (d, 12H).

LCMS: m/z 312.2 (M+H)⁺ (ES⁺).

### Step I: 5-(4-fluoro-2,6-diisopropylbenzyl)-4H-1,2,4-triazole-3-thiol

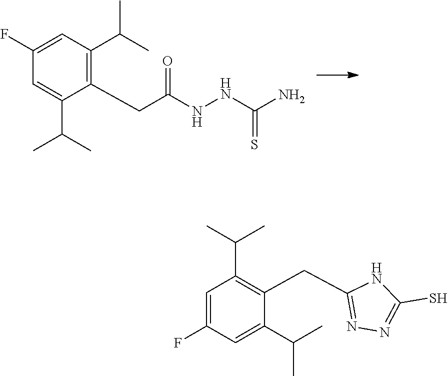 [SMILES: CC(C)c1cc(F)cc(C(C)C)c1CC(=O)NNC(N)=S ; CC(C)c1cc(F)cc(C(C)C)c1Cc1nnc(S)[nH]1]

A solution of 2-(2-(4-fluoro-2,6-diisopropylphenyl)acetyl)hydrazinecarbothioamide (250 mg, 802.78 μmol, 1 eq) in aqueous NaOH solution (10 mL, 1.25 M, 15.57 eq) was stirred at 100° C. for 2 hours. The reaction mixture was adjusted to pH 4 with 1N aqueous HCl solution. Solids were formed and the suspension mixture was filtered. The filter cake was collected to give the title compound (170 mg, 72.18% yield) as a yellow solid.

¹H NMR (400 MHz, DMSO-d₆): δ13.24 (br s, 1H), 13.18 (s, 1H), 6.95-6.93 (m, 2H), 3.96 (s, 2H), 3.09-3.04 (m, 2H) and 1.12-1.10 (m, 12H).

LCMS: m/z 294.2 (M+H)⁺ (ES⁺).

### Intermediate R3: 5-((1,2,3,5,6,7-hexahydro-s-indacen-4-yl)methyl)-4H-1,2,4-triazole-3-thiol

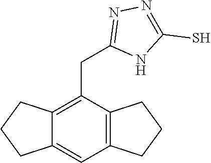 [SMILES: Sc1nnc(Cc2c3c(cc4c2CCC4)CCC3)[nH]1]

### Step A: 3-chloro-1-(2,3-dihydro-1H-inden-5-yl)propan-1-one

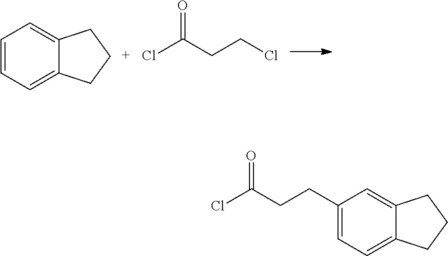

A suspension of AlCl₃ (225.67 g, 1.69 mol, 1 eq) in DCM (1 L) was cooled to −10° C. under N₂ atmosphere. Then a mixture of 2,3-dihydro-1H-indene (200 g, 1.69 mol, 1 eq) and 3-chloropropanoyl chloride (214.88 g, 1.69 mol, 1 eq) in DCM (400 mL) was added dropwise to the above suspension. After addition, the mixture was warmed to 27° C. and stirred for 2 hours. The reaction mixture was added slowly to an aqueous HCl solution (2 N, 2.8 L) below 10° C. The layers were separated and the aqueous layer was extracted with DCM (1). The combined organic layers were washed with water (1 L), saturated aqueous NaHCO₃ solution (1 L) and brine (500 mL). The organic layer was dried over anhydrous Na₂SO₄, filtered and concentrated in vacuum. The residue was triturated with petroleum ether (500 mL) to give the title compound (260.44 g, 73.74% yield) as a white solid.

¹H NMR (400 MHz, CDCl₃): δ 7.84 (s, 1H), 7.79-7.76 (m, 1H), 7.34-7.32 (d, 1H), 3.96-3.93 (t, 2H), 3.48-3.44 (t, 2H), 3.00-2.96 (t, 4H), 2.18-2.11 (m, 2H).

LCMS: m/z 209.1 (M+H)⁺ (ES⁺).

### Step B: 2,3,6,7-tetrahydro-s-indacen-1 (5H)-one

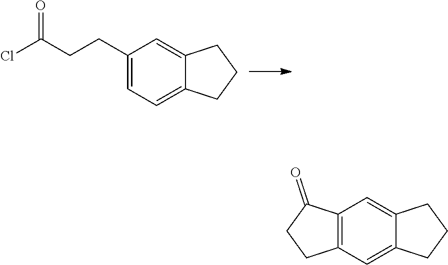 [SMILES: O=C(Cl)CCc1ccc2c(c1)CCC2 ; O=C1CCc2cc3c(cc21)CCC3]

To concentrated H₂SO₄ (1.84 kg, 18.39 mol, 98 wt. % in aqueous solution, 37.25 eq) was added 3-chloro-1-(2,3-dihydro-1H-inden-5-yl)propan-1-one (103 g, 493.57 mmol, 1 eq). Then the reaction mixture was stirred at 70° C. for 12 hours. The reaction mixture was poured into ice-water (4.5 L) and the resulting suspension was filtered. The filter cake was dissolved in EtOAc (500 mL) and washed with saturated aqueous Na₂CO₃ solution (500 mL). The organic layer was separated and the aqueous layer was extracted with EtOAc (3×200 mL). The combined organic layers were concentrated in vacuum to give the title compound (60 g, 69.17% yield, 98% purity on LCMS) as a yellow solid.

¹H NMR (400 MHz, CDCl₃): δ 7.58 (s, 1H), 7.30 (s, 1H), 3.08-2.96 (m, 2H), 2.95-2.91 (m, 4H), 2.70 (t, 2H) and 2.15-2.05 (m, 2H).

LCMS: m/z 173.2 (M+H)⁺ (ES⁺).

### Step C: 1,2,3,5,6,7-hexahydro-s-indacene

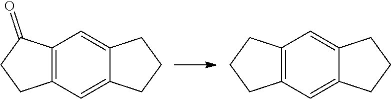 [SMILES: O=C1CCc2cc3c(cc21)CCC3 ; c1c2c(cc3c1CCC3)CCC2]

To a solution of 2,3,6,7-tetrahydro-s-indacen-1 (5H)-one (15 g, 87.10 mmol, 1 eq) in MeOH (200 mL) was added with CH₃SO₃H (16.94 g, 176.22 mmol, 2.02 eq) and Pd(OH)₂/C (3.2 g, 20 wt. % loading on activated carbon). The reaction mixture was degassed and purged with H₂ three times. The resulting mixture was stirred at 25° C. for 12 hours under H₂ (15 psi). The reaction mixture was filtered, and the filtrate was concentrated in vacuum. The residue was purified by column chromatography (SiO₂, only eluting with petroleum ether) to give the title compound (12 g, 84.65% yield) as a white solid.

¹H NMR (400 MHz, CDCl₃): δ 7.14 (s, 2H), 3.00-2.85 (m, 8H) and 2.16-2.09 (m, 4H).

### Step D: 4-bromo-1,2,3,5,6,7-hexahydro-s-indacene

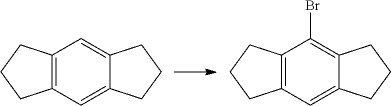 [SMILES: c1c2c(cc3c1CCC3)CCC2 ; Brc1c2c(cc3c1CCC3)CCC2]

To a solution of 1,2,3,5,6,7-hexahydro-s-indacene (11.5 g, 72.67 mmol, 1 eq) in CCl₄ (200 mL) was added I₂ (922 mg, 3.63 mmol, 0.05 eq). Then a solution of Br₂ (12.19 g, 76.31 mmol, 1.05 eq) in CCl₄ (50 mL) was added dropwise to the above mixture at 0° C. The resulting mixture was stirred at 0° C. for 2 hours. The reaction mixture was quenched with saturated aqueous NH₄Cl solution (100 mL) and extracted with DCM (3×200 mL). The organic layers were dried over anhydrous Na₂SO₄, filtered and concentrated in vacuum. The residue was purified by column chromatography (SiO₂, only eluting with petroleum ether) to give the title compound (15 g, 87.04% yield) as a red oil.

¹H NMR (400 MHz, CDCl₃): δ 7.00 (s, 1H), 3.10-2.83 (m, 8H) and 2.11 (m, 4H)

### Step E: tert-butyl 2-(1,2,3,5,6,7-hexahydro-s-indacen-4-yl)acetate

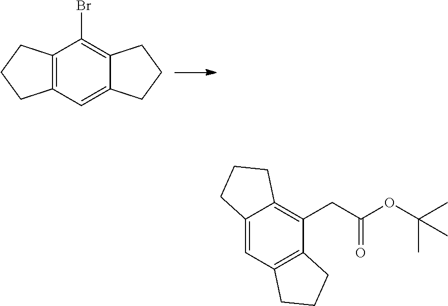 [SMILES: Brc1c2c(cc3c1CCC3)CCC2 ; CC(C)(C)OC(=O)Cc1c2c(cc3c1CCC3)CCC2]

To a solution of 4-bromo-1,2,3,5,6,7-hexahydro-s-indacene (20 g, 84.34 mmol, 1 eq), Pd₂(dba)₃ (3.86 g, 4.22 mmol, 0.05 eq) and XPhos (4.02 g, 8.43 mmol, 0.1 eq) in THF (1 mL) was added (2-(tert-butoxy)-2-oxoethyl)zinc(II) bromide (168.68 mmol, 500 mL, 0.5 M, in THF, 2 eq) at 0° C. After addition, the reaction mixture was stirred at 70° C. for 12 hours. The reaction mixture was quenched with saturated aqueous NH₄C solution (500 mL) and extracted with ethyl acetate (3×500 mL). The organic layers were dried over anhydrous Na₂SO₄, filtered and concentrated in vacuum. The residue was purified by column chromatography (SiO₂, only eluting with petroleum ether) to give the title compound (20 g, 87.06% yield) as a yellow oil.

¹H NMR (400 MHz, CDCl₃): 7.02 (s, 1H), 3.51 (s, 2H), 2.90-2.84 (m, 8H), 2.11-2.04 (m, 4H) and 1.44 (s, 9H).

### Step F: 2-(1,2,3,5,6,7-hexahydro-s-indacen-4-yl)acetic acid

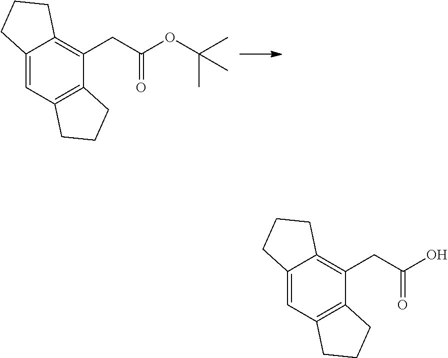 [SMILES: CC(C)(C)OC(=O)Cc1c2c(cc3c1CCC3)CCC2 ; O=C(O)Cc1c2c(cc3c1CCC3)CCC2]

To a solution of tert-butyl 2-(1,2,3,5,6,7-hexahydro-s-indacen-4-yl)acetate (20 g, 73.43 mmol, 1 eq) in DCM (200 mL) was added TFA (308 g, 2.70 mol, 36.79 eq). The reaction mixture was stirred at 25° C. for 1 hour. The reaction mixture was concentrated in vacuum. The residue was dissolved into DCM (300 mL) and the mixture was adjusted to pH 8-9 with an aqueous NaOH solution (2 N). The aqueous phase was adjusted to pH 2-3 with an aqueous HCl solution (1 N). Large white solids were formed, and the mixture was filtered. The collected solid was dried to give the title compound (12 g, 75.57% yield) as a white solid.

¹H NMR (400 MHz, DMSO-d₆): δ 12.20 (s, 1H), 6.95 (s, 1H), 3.70 (s, 2H), 2.82-2.70 (m, 8H) and 2.03-1.94 (m, 4H).

### Step G: 2-(1,2,3,5,6,7-hexahydro-s-indacen-4-yl)acetyl chloride

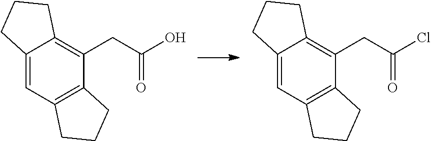 [SMILES: O=C(O)Cc1c2c(cc3c1CCC3)CCC2 ; O=C(Cl)Cc1c2c(cc3c1CCC3)CCC2]

A solution of 2-(1,2,3,5,6,7-hexahydro-s-indacen-4-yl)acetic acid (10 g, 46.24 mmol, 1 eq) in SOCl₂ (82 g, 689.25 mmol, 14.91 eq) was stirred at 50° C. for 1 hour. The reaction mixture was concentrated in vacuum to give the title compound (10.85 g, 100.00% yield) as a yellow oil, which was used directly in the next step.

### Step H

### 2-(2-(1,2,3,5,6,7-hexahydro-s-indacen-4-yl)acetyl)hydrazinecarbothioamide

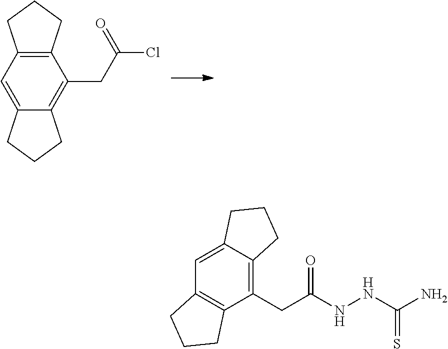 [SMILES: O=C(Cl)Cc1c2c(cc3c1CCC3)CCC2 ; NC(=S)NNC(=O)Cc1c2c(cc3c1CCC3)CCC2]

To a mixture of hydrazinecarbothioamide (4 g, 43.89 mmol, 1 eq) in pyridine (49 g, 619.47 mmol, 14.11 eq) was added dropwise a solution of 2-(1,2,3,5,6,7-hexahydro-s-indacen-4-yl)acetyl chloride (10.82 g, 46.09 mmol, 1.05 eq) in toluene (50 mL) at 0° C. After addition, the mixture was warmed to 25° C., and stirred for 12 hours. The reaction mixture was concentrated in vacuum. The residue was triturated with a mixture of petroleum ether and ethyl acetate (20 mL, V:V=1:1) to give the title compound (8 g, 62.98% yield) as a yellow solid.

¹H NMR (400 MHz, DMSO-d₆): δ 6.91 (d, 1H), 3.47 (s, 2H), 2.79-2.74 (m, 8H) and 1.98-1.96 (m, 4H). Four exchangeable protons not observed.

LCMS: m/z 290.1 (M+H)⁺ (ES⁺).

### Step I: 5-((1,2,3,5,6,7-hexahydro-s-indacen-4-yl)methyl)-4H-1,2,4-triazole-3-thiol

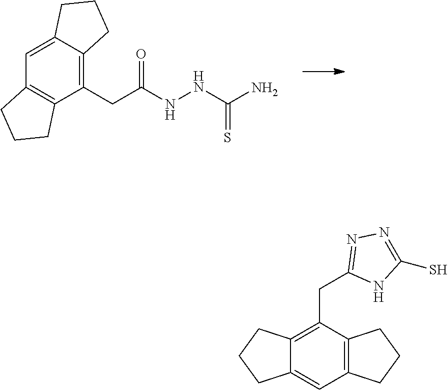 [SMILES: NC(=S)NNC(=O)Cc1c2c(cc3c1CCC3)CCC2 ; Sc1nnc(Cc2c3c(cc4c2CCC4)CCC3)[nH]1]

To a mixture of 2-(2-(1,2,3,5,6,7-hexahydro-s-indacen-4-yl)acetyl)hydrazine-carbothioamide (3 g, 10.37 mmol, 1 eq) in H₂O (100 mL) was added NaOH (5 g, 125.01 mmol, 12.06 eq). The reaction mixture was stirred at 100° C. for 5 hours. The reaction mixture was adjusted to pH 3˜4 with an aqueous HCl solution (1 N) and solids were formed. The mixture was filtered and the solids were collected to give the title compound (1.2 g, 26.02% yield, 61% purity on LCMS) as a yellow solid, which was used directly in the next step.

¹H NMR (400 MHz, DMSO-d₆): δ13.30-13.07 (m, 2H), 6.98 (s, 1H), 3.87 (s, 2H), 2.77-2.74 (m, 8H) and 1.99-1.92 (m, 4H).

LCMS: m/z 272.2 (M+H)⁺ (ES⁺).

### Intermediate R4: 5-(2-methoxypyridin-4-yl)-2,3-dihydro-1H-inden-4-amine

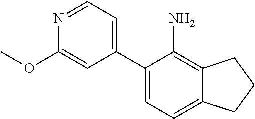 [SMILES: COc1cc(-c2ccc3c(c2N)CCC3)ccn1]

### Step A: 4-nitro-2,3-dihydro-1H-indene

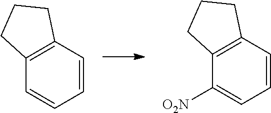 [SMILES: c1ccc2c(c1)CCC2 ; O=[N+]([O-])c1cccc2c1CCC2]

To a mixture of 2,3-dihydro-1H-indene (60 g, 507.72 mmol, 1 eq) in concentrated H₂SO₄ (30 mL) was added a solution of HNO₃ (50 mL, 69 wt. % in aqueous solution) in concentrated H₂SO₄ (50 mL) dropwise at 0° C. over a period of 3.5 hours. The reaction mixture was stirred at 0° C. for 0.5 hour. Then the reaction mixture was poured into ice water (600 mL) and extracted with EtOAc (2×400 mL). The combined organic layers were washed with water (500 mL), saturated aqueous NaHCO₃ solution (500 mL) and brine (2×500 mL). The organic layer was dried over anhydrous Na₂SO₄, filtered and concentrated. The residue was purified by column chromatography (SiO₂, petroleum ether:ethyl acetate 1:0 to 100:1) to give the title compound (55 g, contained another regio-isomer) as a colourless oil.

¹H NMR (400 MHz, CDCl₃): δ 7.98 (d, 1H), 7.51 (d, 1H), 7.30 (t, 1H), 3.41 (t, 2H), 302 (t, 2H) and 2.22-2.20 (m, 2H).

### Step B: 2,3-dihydro-1H-inden-4-amine

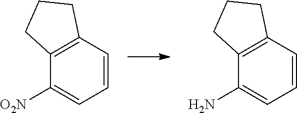 [SMILES: O=[N+]([O-])c1cccc2c1CCC2 ; Nc1cccc2c1CCC2]

To a solution of 4-nitro-2,3-dihydro-1H-indene (55 g, contained another regio-isomer) in MeOH (500 mL) was added Pd/C (5 g, 10 wt. % loading on activated carbon) under N₂. The suspension was degassed in vacuum and purged with H₂ several times. The reaction mixture was stirred at 20° C. for 12 hours under H₂ (50 psi). The reaction mixture was filtered and the filtrate was concentrated in vacuum. The residue was purified by column chromatography (SiO₂, petroleum ether:ethyl acetate 1:0 to 100:4) to give the title compound (19.82 g, 4255% yield, 96.39% purity on LCMS) as a brown oil.

¹H NMR (400 MHz, CDCl₃): δ 7.01 (t, 1H), 6.71 (d, 1H), 6.51 (d, 1H), 3.57 (br s, 2H), 2.93 (t, 2H), 2.75 (t, 2H) and 2.16-2.08 (m, 2H).

LCMS: m/z 134.2 (M+H)⁺ (ES⁺).

### Step C: N-(2,3-dihydro-1H-inden-4-yl)acetamide

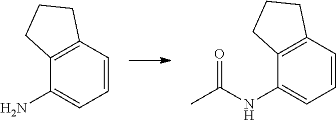

To a solution of 2,3-dihydro-1H-inden-4-amine (19.8 g, 148.66 mmol, 1 eq) and TEA (19.56 g, 193.26 mmol, 1.3 eq) in DCM (300 mL) was added dropwise Ac₂O (17.45 g, 170.96 mmol, 1.15 eq) at 0° C. over 0.1 hours. Then the reaction mixture was warmed to 16° C. and stirred for 1.4 hours. The mixture was poured into water (500 mL) and extracted with DCM (2×300 mL). The combined organic phases were washed with brine (2×500 mL), dried over anhydrous Na₂SO₄, filtered and concentrated in vacuum to give the title compound (25.74 g, 95.54% yield, 96.69% purity on LCMS) as a white solid.

¹H NMR (400 MHz, CDCl₃): δ 7.70 (d, 1H), 7.15 (t, 1H), 7.02 (d, 1H), 2.95 (t, 2H), 2.81 (t, 2H), 2.18 (s, 3H) and 2.15-2.08 (m, 2H). One exchangeable proton not observed.

LCMS: m/z 176.2 (M+H)⁺ (ES⁺).

### Step D: N-(5-bromo-2,3-dihydro-1H-inden-4-yl)acetamide

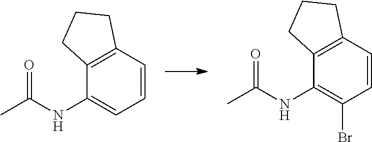

A mixture of N-(2,3-dihydro-1H-inden-4-yl)acetamide (34.6 g, 197.46 mmol, 1 eq), 4-methylbenzenesulfonic acid (18.70 g, 108.60 mmol, 0.55 eq) and Pd(OAc)₂ (2.22 g, 9.87 mmol, 0.05 eq) were suspended in toluene (400 mL) and then stirred at 20° C. for 0.5 hours under air atmosphere. NBS (38.66 g, 217.20 mmol, 1.1 eq) was added into the above mixture. The resulting reaction mixture was stirred at 20° C. for 2 hours. The reaction mixture was poured into water (500 mL) and extracted with EtOAc (2×500 mL). The combined organic phases were washed with brine (2×500 mL), dried over anhydrous Na₂SO₄, filtered and concentrated in vacuum. The residue was purified by column chromatography (SiO₂, petroleum ether:ethyl acetate 10:1 to 2:1) to give the title compound (13.9 g, 27.17% yield, 98.1% purity on LCMS) as a white solid.

¹H NMR (400 MHz, CDCl₃): δ 7.33 (d, 1H), 7.16 (s, 1H), 6.98 (d, 1H), 2.92-2.83 (m, 4H), 2.21 (s, 3H) and 2.10-2.02 (m, 2H).

LCMS: m/z 254.1 (M+H)⁺ (ES⁺).

### Step E: 5-bromo-2,3-dihydro-1H-inden-4-amine

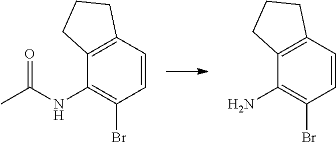

A mixture of N-(5-bromo-2,3-dihydro-1H-inden-4-yl)acetamide (45.68 g, 179.76 mmol, 1 eq) in EtOH (200 mL) and concentrated HCl (300 mL) was stirred at 80° C. for 36 hours. The reaction mixture was cooled to 0° C. in an ice bath and solid precipitated out. The suspension was filtered. The filter cake was washed with ice water (50 mL) and dried in vacuum to give the title compound (34.1 g, 71.81% yield, 94.08% purity on LCMS, HCl salt) as a grey solid.

¹H NMR (400 MHz, DMSO-d₆): δ 7.67 (br s, 2H), 7.24 (d, 1H), 6.69 (d, 1H), 2.85 (t, 2H), 2.79 (t, 2H) and 2.04-1.96 (m, 2H).

LCMS: m/z 212.0 (M+H)⁺ (ES⁺).

### Step F: 5-(2-methoxypyridin-4-yl)-2,3-dihydro-1H-inden-4-amine

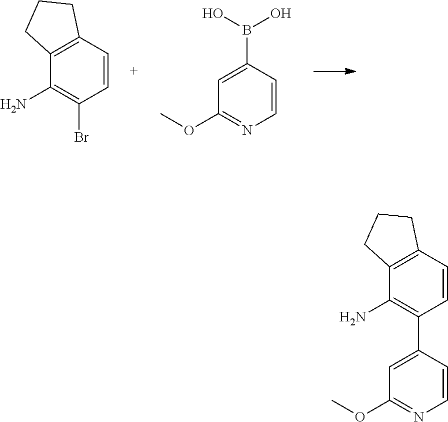 [SMILES: Nc1c(Br)ccc2c1CCC2 ; COc1cc(B(O)O)ccn1 ; COc1cc(-c2ccc3c(c2N)CCC3)ccn1]

A mixture of (2-methoxypyridin-4-yl)boronic acid (25.11 g, 164.15 mmol, 1.2 eq), 5-bromo-2,3-dihydro-1H-inden-4-amine (34 g, 136.80 mmol, 1 eq, HCl salt) and K₂CO₃ (60.50 g, 437.74 mmol, 3.2 eq) in dioxane (500 mL) and H₂O (100 mL) was degassed with N₂ for 15 minutes before Pd(dppf)Cl₂.CH₂Cl₂ (6 g, 7.35 mmol, 0.053 eq) was added. The reaction mixture was heated to 80° C. for 12 hours under N₂. The mixture was poured into water (500 mL) and extracted with EtOAc (2×500 mL). The combined organic phases were washed with brine (2×700 mL), dried over anhydrous Na₂SO₄, filtered and concentrated in vacuum. The residue was purified by column chromatography (SiO₂, petroleum ether:ethyl acetate 0:1 to 1:10) to give the title compound (27.4 g, 79.19% yield, 95% purity on LCMS) as a white solid.

¹H NMR (400 MHz, CDCl₃): δ 8.22 (d, 1H), 7.03-7.00 (m, 1H), 6.99 (d, 1H), 6.87 (s, 1H), 6.77 (d, 1H), 3.99 (s, 3H), 3.77 (br s, 2H), 2.97 (t, 2H), 2.77 (t, 2H) and 2.21-2.13 (m, 2H).

LCMS: m/z 241.2 (M+H)⁺ (ES⁺).

### Intermediate R5: 5-((5-(2-methoxypyridin-4-yl)-2,3-dihydro-1H-inden-4-yl)methyl)-4H-1,2,4-triazole-3-thiol

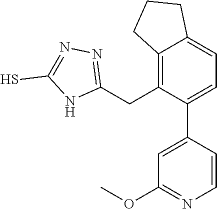 [SMILES: COc1cc(-c2ccc3c(c2Cc2nnc(S)[nH]2)CCC3)ccn1]

### Step A: 4-(4-bromo-2,3-dihydro-1H-inden-5-yl)-2-methoxypyridine

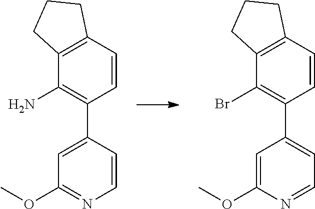 [SMILES: COc1cc(-c2ccc3c(c2N)CCC3)ccn1 ; COc1cc(-c2ccc3c(c2Br)CCC3)ccn1]

To a mixture of 5-(2-methoxypyridin-4-yl)-2,3-dihydro-1H-inden-4-amine (Intermediate R4) (28 g, 116.52 mmol, 1 eq) in MeCN (300 mL) was added isopentyl nitrite (16.38 g, 139.83 mmol, 1.2 eq) at 0° C. The reaction mixture was stirred at 0° C. for 30 minutes under N₂. CuBr (17.05 g, 118.85 mmol, 1.02 eq) was added into the above mixture at 0° C. and the resulting mixture was stirred at 60° C. for 1 hour. The reaction mixture was filtered and the filtrate was concentrated in vacuum. The residue was purified by column chromatography (SiO₂, petroleum ether:ethyl acetate 1:0 to 20:1) to give the title compound (15 g, 37.10% yield, 87.66% purity on LCMS) as a yellow solid.

¹H NMR (400 MHz, CDCl₃): δ 8.20 (d, 1H), 7.21 (d, 1H), 7.06 (d, 1H), 6.94 (dd, 1H), 6.78 (s, 1H), 3.99 (s, 3H), 3.08 (t, 2H), 3.03 (t, 2H) and 2.20-2.10 (m, 2H).

LCMS: m/z 304.0 (M+H)⁺ (ES⁺).

### Step B: tert-butyl 2-(5-(2-methoxypyridin-4-yl)-2,3-dihydro-1H-inden-4-yl)acetate

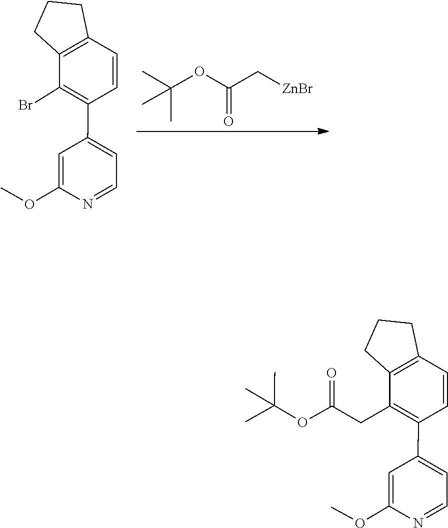

To a mixture of 4-(4-bromo-2,3-dihydro-1H-inden-5-yl)-2-methoxypyridine (15 g, 49.31 mmol, 1 eq), XPhos (2.35 g, 4.93 mmol, 0.1 eq) and Pd₂(dba)₃ (2.26 g, 2.47 mmol, 0.05 eq) in THF (50 mL) was added a solution of (2-(tert-butoxy)-2-oxoethyl) zinc (II) bromide in THF (0.5 M, 296 mL, 3 eq) at 20° C. under N₂. The reaction mixture was stirred at 70° C. for 12 hours under N₂. The mixture was poured into saturated aqueous NH₄Cl solution (200 mL). The aqueous phase was extracted with EtOAc (3×200 mL). The combined organic phases were washed with brine (20 mL), dried over anhydrous Na₂SO₄, filtered and concentrated in vacuum. The residue was purified by column chromatography (SiO₂, petroleum ether:ethyl acetate 1:0 to 20:1) to give the title compound (15 g, 83.26% yield, 92.91% purity on LCMS) as a yellow oil.

¹H NMR (400 MHz, CDCl₃): δ 8.17 (dd, 1H), 7.20 (d, 1H), 7.04 (d, 1H), 6.86 (dd, 1H), 6.72 (s, 1H), 3.98 (s, 3H), 3.47 (s, 2H), 3.01 (t, 2H), 2.90 (t, 2H), 2.18-2.10 (m, 2H) and 1.43 (s, 9H).

LCMS: m/z 340.1 (M+H)⁺ (ES⁺).

### Step C: 2-(5-(2-methoxypyridin-4-yl)-2,3-dihydro-1H-inden-4-yl)acetic acid

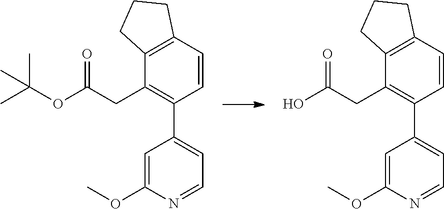 [SMILES: COc1cc(-c2ccc3c(c2CC(=O)OC(C)(C)C)CCC3)ccn1 ; COc1cc(-c2ccc3c(c2CC(=O)O)CCC3)ccn1]

To a solution of tert-butyl 2-(5-(2-methoxypyridin-4-yl)-2,3-dihydro-1H-inden-4-yl)acetate (16 g, 47.14 mmol, 1 eq) in DCM (100 mL) was added TFA (154 g, 1.35 mol, 28.65 eq) at 20° C. The reaction mixture was stirred at 20° C. for 12 hours. The mixture was concentrated under reduced pressure. The residue was purified by column chromatography (SiO₂, petroleum ether:ethyl acetate 20:1 to 5:1) to give the title compound (12 g, 87.17% yield, 97.02% purity on LCMS) as a yellow solid.

¹H NMR (400 MHz, CDCl₃): δ 8.89 (br s, 1H), 8.35 (d, 1H), 7.25 (s, 1H), 7.11 (d, 1H), 7.05 (d, 1H), 6.98 (s, 1H), 4.05 (s, 3H), 3.58 (s, 2H), 3.00 (t, 2H), 2.92 (t, 2H) and 2.19-2.10 (m, 2H).

LCMS: m/z 284.1 (M+H)⁺ (ES⁺).

### Step D: 2-(5-(2-methoxypyridin-4-yl)-2,3-dihydro-1H-inden-4-yl)acetyl chloride

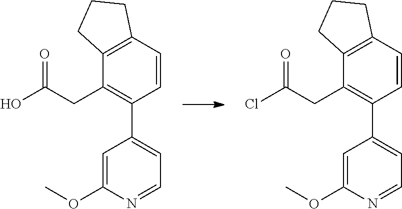 [SMILES: COc1cc(-c2ccc3c(c2CC(=O)O)CCC3)ccn1 ; COc1cc(-c2ccc3c(c2CC(=O)Cl)CCC3)ccn1]

To a solution of 2-(5-(2-methoxypyridin-4-yl)-2,3-dihydro-1H-inden-4-yl)acetic acid (4 g, 14.12 mmol, 1 eq) and DMF (51 mg, 705.91 μmol, 0.05 eq) in DCM (30 mL) was added oxalyl chloride (8.96 g, 70.59 mmol, 5 eq) at 0° C. under N₂. The reaction mixture was stirred at 20° C. for 0.5 hour. The reaction mixture was concentrated in vacuum to give the title compound (4.3 g, crude) as a yellow oil, which was used in the next step without further purification.

### Step E: 2-(2-(5-(2-methoxypyridin-4-yl)-2,3-dihydro-1H-inden-4-yl) acetyl) hydrazinecarbothioamide

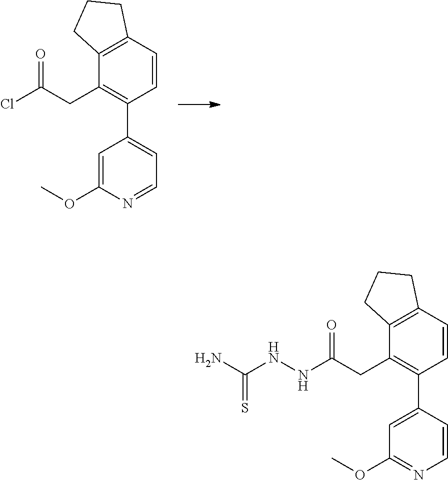 [SMILES: COc1cc(-c2ccc3c(c2CC(=O)Cl)CCC3)ccn1 ; COc1cc(-c2ccc3c(c2CC(=O)NNC(N)=S)CCC3)ccn1]

To a solution of aminothiourea (1.30 g, 14.25 mmol, 1 eq), pyridine (40 mL) and DCM (40 mL) was added 2-(5-(2-methoxypyridin-4-yl)-2,3-dihydro-1H-inden-4-yl)acetyl chloride (4.3 g, 14.25 mmol, 1 eq) at 0° C. The reaction mixture was stirred at 20° C. for 12 hours. The reaction mixture was concentrated in vacuum. The residue was treated with water (20 mL) and the mixture was stirred for 30 minutes. Solid was precipitated. The solid was collected and triturated with a mixture of petroleum ether and ethyl acetate (5 mL, v:v 1:1) for 15 minutes. The solid was collected and dried to give the title compound (1.6 g, 31.8% yield over two steps, 90.4% purity on LCMS) as a yellow solid.

¹H NMR (400 MHz, DMSO-d₆): δ 9.87 (br s, 1H), 9.30 (br s, 1H), 8.17 (d, 1H), 7.87 (br s, 1H), 7.42 (br s, 1H), 7.41 (d, 1H), 7.01 (d, 1H), 6.95 (d, 1H), 6.80 (s, 1H), 3.87 (s, 3H), 3.43 (s, 2H), 2.93-2.83 (m, 4H) and 2.06-1.98 (m, 2H).

LCMS: m/z 357.1 (M+H)⁺.

### Step F: 5-((5-(2-methoxypyridin-4-yl)-2,3-dihydro-1H-inden-4-yl)methyl)-4H-1,2,4-triazole-3-thiol

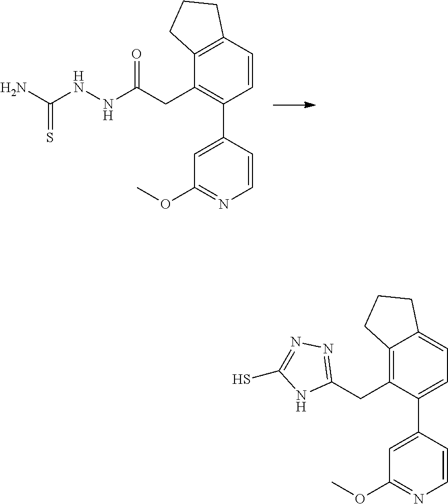 [SMILES: COc1cc(-c2ccc3c(c2CC(=O)NNC(N)=S)CCC3)ccn1 ; COc1cc(-c2ccc3c(c2Cc2nnc(S)[nH]2)CCC3)ccn1]

A mixture of 2-(2-(5-(2-methoxypyridin-4-yl)-2,3-dihydro-1H-inden-4-yl)acetyl)-hydrazinecarbothioamide (1.6 g, 4.49 mmol, 1 eq) and an aqueous NaOH solution (35-91 g, 44.89 mmol, 5 wt. % in aqueous solution, 10 eq) was stirred at 100° C. for 2 hours. The mixture was adjusted to pH 2 with an aqueous HCl solution (1 M) at 0° C. Solid precipitated out. The solid was collected and dried to give the title compound (1.1 g, 72.41% yield) as a yellow solid.

¹H NMR (DMSO-d₆): δ 13.22 (br s, 1H), 13.13 (br s, 1H), 8.19 (d, 1H), 7.25 (d, 1H), 7.05 (d, 1H), 6.91 (d, 1H), 6.75 (s, 1H), 3.88 (s, 3H), 3.77 (s, 2H), 2.94-2.90 (m, 2H), 2.74-2.67 (m, 2H) and 2.06-2.00 (m, 2H).

LCMS: m/z 339.1 (M+H)⁺.

### Intermediate R6:5-((5-(2-methoxypyridin-4-yl)-2,3-dihydro-1H-inden-4-yl)amino)-4H-1,2,4-triazole-3-thiol

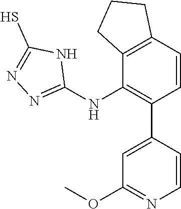 [SMILES: COc1cc(-c2ccc3c(c2Nc2nnc(S)[nH]2)CCC3)ccn1]

### Step A: 4-(4-isothiocyanato-2,3-dihydro-1H-inden-5-yl)-2-methoxypyridine

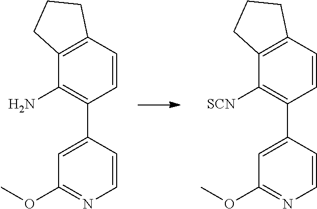

To a stirred solution of thiocarbonyl dichloride (3.83 g, 33.29 mmol, 2 eq) and CaCO₃ (3.33 g, 33.29 mmol, 2 eq) in DCM (40 mL) and H₂O (20 mL) was added dropwise a solution of 5-(2-methoxypyridin-4-yl)-2,3-dihydro-1H-inden-4-amine (Intermediate R⁴) (4 g, 16.65 mmol, 1 eq) in DCM (20 mL) at 16° C. Then the reaction mixture was stirred at 16° C. for 2 hours. The mixture was poured into water (200 mL) and extracted with DCM (2×200 mL). The combined organic phases were washed with brine (2×300 mL), dried over anhydrous Na₂SO₄, filtered and concentrated in vacuum. The residue was purified by column chromatography (SiO₂, petroleum ether:ethyl acetate 1:0 to 20:1) to give the title compound (5.4 g, 96.51% yield, 84% purity on LCMS) as a white solid.

¹H NMR (400 MHz, CDCl₃): δ 8.24 (d, 1H), 7.22-7.14 (m, 2H), 6.99 (dd, 1H), 6.83 (s, 1H), 4.01 (s, 3H), 3.10 (t, 2H), 3.01 (t, 2H) and 2.23-2.15 (m, 2H).

LCMS: m/z 283.1 (M+H)⁺ (ES⁺).

### Step B: 1-carbamothioyl-3-[5-(2-methoxy-4-pyridyl)indan-4-yl]thiourea

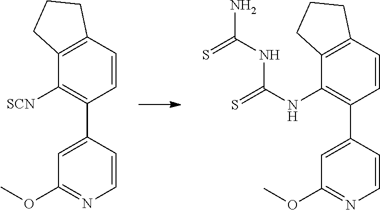

To a stirred solution of thiourea (2.16 g, 28.33 mmol, 2 eq) in THF (50 mL) was added t-BuONa (2.04 g, 21.25 mmol, 1.5 eq). The reaction mixture was stirred at 25° C. for 16 hours. Then to this mixture was added 4-(4-isothiocyanato-2,3-dihydro-1H-inden-5-yl)-2-methoxypyridine (4 g, 14.17 mmol, 1 eq) and the resulting reaction mixture was stirred at 25° C. for 1 hour. The reaction mixture was poured into EtOAc (200 mL) and acidified to pH 7 with an aqueous HCl solution (6 N). The organic layer was separated and dried over anhydrous Na₂SO₄, filtered and concentrated in vacuum to give the title compound (7 g, 99.24% yield, 72% purity on LCMS) as a light yellow solid. LCMS: m/z 359.2 (M+H)⁺ (ES⁺).

Step C: ethyl N′-(5-(2-methoxypyridin-4-yl)-2,3-dihydro-1H-inden-4-yl)carbamothioyl-carbamimidothioate and ethyl N′-carbamothioyl-N-(5-(2-methoxypyridin-4-yl)-2,3-dihydro-1H-inden-4-yl)carbamimidothioate

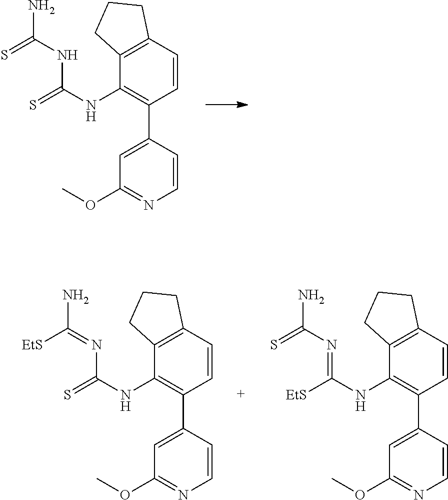

To a stirred solution of 1-carbamothioyl-3-[5-(2-methoxy-4-pyridyl)indan-4-yl]thiourea (7 g, 14.06 mmol, 1 eq) in DMF (70 mL) was added TEA (2.85 g, 28.12 mmol, 2 eq). The resulting suspension was stirred at 20° C. for 0.5 hour and then to this mixture was added EtI (1.86 g, 11.95 mmol, 0.85 eq). The reaction mixture was stirred at 20° C. for 0.5 hour. The reaction mixture was poured into water (500 mL) and extracted with EtOAc (2×400 mL). The combined organic phases were washed with brine (3×600 mL), dried over anhydrous Na₂SO₄, filtered and concentrated in vacuum. The residue was purified by column chromatography (SiO₂, petroleum ether:ethyl acetate 20:1 to 12:1) to give the title compound (3.4 g, mixture) as a light yellow solid.

¹H NMR (400 MHz, DMSO-d₆): δ 9.91 (br s, 1H), 9.75 (s, 1H), 9.25 (br s, 1H), 8.11 (d, 1H), 7.14 (d, 1H), 7.07 (m, 1H), 6.94 (d, 1H), 6.74 (s, 1H), 3.84 (s, 3H), 2.96-2.88 (m, 4H), 2.66 (t, 2H), 2.06-2.01 (m, 2H) and 1.02 (t, 3H).

LCMS: m/z 387.1 (M+H)⁺ (ES⁺).

### Step D: 5-((5-(2-methoxypyridin-4-yl)-2,3-dihydro-1H-inden-4-yl)amino)-4H-1,2,4-triazole-3-thiol

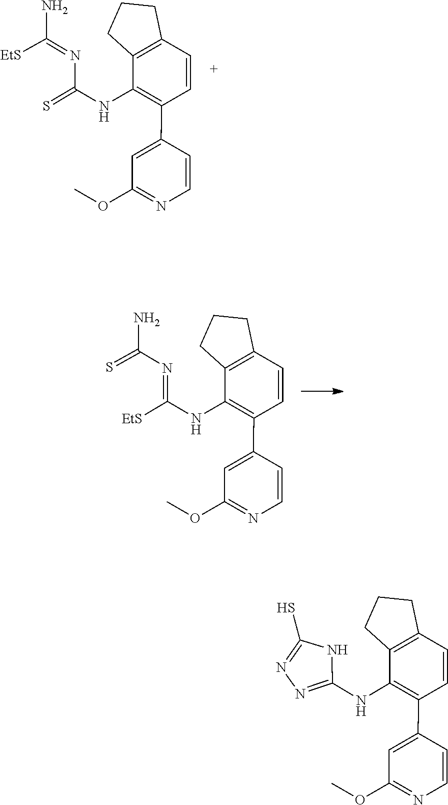

To a mixture of ethyl N′-(5-(2-methoxypyridin-4-yl)-2,3-dihydro-1H-inden-4-yl)-carbamothioylcarbamimidothioate and ethyl N′-carbamothioyl-N-(5-(2-methoxypyridin-4-yl)-2,3-dihydro-1H-inden-4-yl)carbamimidothioate (3.2 g, 8.28 mmol, 1 eq) in EtOH (27 mL) and AcOH (3 mL) was added NH₂NH₂.H₂O (12.69 g, 248.36 mmol, 98% wt. % in aqueous solution, 30 eq). The reaction mixture was stirred at 80° C. for 2 hours. The reaction mixture was concentrated in vacuum. The residue was purified by reversed phase flash chromatography (0.1% NH₃.H₂O-MeCN) to give the title compound (300 mg, 7.9% yield over two steps, 100% purity on LCMS) as a white solid.

¹H NMR (400 MHz, DMSO-d₆): δ 8.12 (d, 1H), 8.08 (s, 1H), 7.17 (d, 1H), 7.12 (d, 1H), 6.94 (dd, 1H), 6.74 (s, 1H), 3.84 (s, 3H), 2.92 (t, 2H), 2.70 (t, 2H) and 2.04-1.97 (m, 2H). Two exchangeable protons not observed.

LCMS: m/z 340.1 (M+H)⁺ (ES⁺).

### Intermediate R7: 7-fluoro-5-(pyridin-4-yl)-2,3-dihydro-1H-inden-4-amine

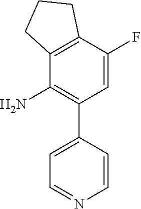 [SMILES: Nc1c(-c2ccncc2)cc(F)c2c1CCC2]

### Step A: 7-fluoro-4-nitro-2,3-dihydro-1H-inden-1-one

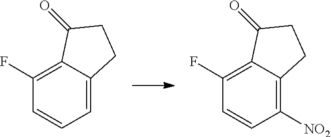 [SMILES: O=C1CCc2cccc(F)c21 ; O=C1CCc2c([N+](=O)[O-])ccc(F)c21]

To a mixture of 7-fluoro-2,3-dihydro-1H-inden-1-one (10 g, 66.60 mmol, 1 eq) in concentrated H₂SO₄ (100 mL) was added dropwise a solution of HNO₃ (86.58 mmol, 5.65 mL, 69 wt. % in aqueous solution, 1.3 eq) in concentrated H₂SO₄ (20 mL) at −15° C. Then the reaction mixture was stirred at −15° C. for 10 minutes. The mixture was quenched with water (1 L) at 0° C. and extracted with EtOAc (3×1). The combined organic phases were dried over anhydrous Na₂SO₄, filtered and concentrated in vacuum. The residue was purified by column chromatography (SiO₂, petroleum ether:ethyl acetate 20:1 to 5:1) to give the title compound (5-4 g, 38% yield, 92.8% purity on LCMS) as a yellow solid.

¹H NMR (400 MHz, CDCl₃): δ 8.51 (dd, 1H), 7.24 (t, 1H), 3.67 (t, 2H) and 2.83 (t, 2H).

### Step B: 7-fluoro-4-nitro-2,3-dihydro-1H-inden-1-ol

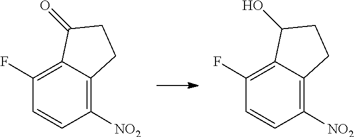 [SMILES: O=C1CCc2c([N+](=O)[O-])ccc(F)c21 ; O=[N+]([O-])c1ccc(F)c2c1CCC2O]

To a mixture of 7-fluoro-4-nitro-2,3-dihydro-1H-inden-1-one (24 g, 122.98 mmol, 1 eq) in MeOH (400 mL) was added NaBH₄ (9.31 g, 245.97 mmol, 2 eq) in portions. Then the reaction mixture was stirred at 15° C. for 0.5 hours. The mixture was poured into the water (500 mL) and extracted with EtOAc (3×500 mL). The combined organic phases were washed with brine (500 mL), dried over anhydrous Na₂SO₄, filtered and concentrated in vacuum to give the title compound (22 g, crude) as brown oil.

¹H NMR (400 MHz, CDCl₃): δ 8.21 (dd, 1H), 7.09 (t, 1H), 5.59-5.52 (m, 1H), 3.64-3.59 (m, 1H), 3.43-3.40 (m, 1H), 2.56-2.51 (m, 1H) and 2.21-2.16 (m, 2H).

### Step C: 4-fluoro-7-nitro-2,3-dihydro-1H-indene

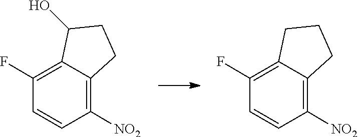 [SMILES: O=[N+]([O-])c1ccc(F)c2c1CCC2O ; O=[N+]([O-])c1ccc(F)c2c1CCC2]

To a mixture of 7-fluoro-4-nitro-2,3-dihydro-1H-inden-1-ol (22 g, 111.58 mmol, 1 eq) in TFA (200 mL) was added Et₃SiH (38.92 g, 334.75 mmol, 3 eq) in one portion. Then the reaction mixture was heated to 50° C. and stirred for 4 hours. The mixture was concentrated in vacuum and then quenched with an aqueous NaOH solution (1 M, 100 mL). Then the mixture was extracted with EtOAc (2×100 mL). The combined organic layers were washed with brine (100 mL), dried over anhydrous Na₂SO₄, filtered and concentrated in vacuum to give the title compound (35 g, crude) as a brown oil.

¹H NMR (400 MHz, CDCl₃): δ 8.06 (dd, 1H), 6.99 (t, 1H), 3.57 (t, 2H), 3.04 (t, 2H) and 2.25-2.18 (m, 2H).

### Step D: 7-fluoro-2,3-dihydro-1H-inden-4-amine

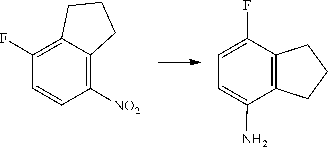 [SMILES: O=[N+]([O-])c1ccc(F)c2c1CCC2 ; Nc1ccc(F)c2c1CCC2]

To a mixture of 4-fluoro-7-nitro-2,3-dihydro-1H-indene (35 g, 193.20 mmol, 1 eq) in MeOH (200 mL) was added Pd/C (5 g, 10 wt. % loading on activated carbon) in one portion at 25° C. under nitrogen. Then the reaction mixture was stirred at 25° C. for 12 hours under H₂ (15 psi). The reaction mixture was filtered and the filtrate was concentrated in vacuum. The residue was purified by column chromatography (SiO₂, petroleum ether:ethyl acetate 50:1 to 10:1) to give the title compound (11.5 g, 61.9% yield over three steps) as a brown solid.

¹H NMR (400 MHz, CDCl₃): δ 6.69 (t, 1H), 6.44 (dd, 1H), 3.45 (br s, 2H), 2.95 (t, 2H), 2.75 (t, 2H) and 2.19-2.11 (m, 2H).

LCMS: m/z 152.2 (M+H)⁺ (ES⁺).

### Step E: 5-bromo-7-fluoro-2,3-dihydro-1H-inden-4-amine

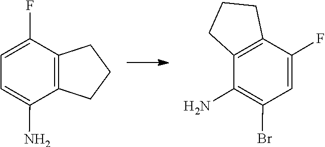 [SMILES: Nc1ccc(F)c2c1CCC2 ; Nc1c(Br)cc(F)c2c1CCC2]

To a solution of 7-fluoro-2,3-dihydro-1H-inden-4-amine (3.5 g, 23.15 mmol, 1 eq) in toluene (40 mL) was added NBS (4.33 g, 24.31 mmol, 1.05 eq) at 25° C. in a single portion. The reaction mixture turned dark brown immediately and then the mixture was stirred at 25° C. for 30 minutes. The reaction mixture was quenched with saturated aqueous Na₂SO₃ solution (150 mL) and extracted with EtOAc (2×150 mL. The combined organic phases were washed with brine (100 mL), dried over anhydrous Na₂SO₄, filtered and concentrated in vacuum. The residue was purified by column chromatography (SiO₂, petroleum ether:ethyl acetate 1:0 to 20:1) to give the title compound (2.87 g, 53% yield) as a brown solid.

¹H NMR (400 MHz, CDCl₃): δ 6.99 (d, 1H), 3.81 (br s, 2H), 2.92 (t, 2H), 2.78 (t, 2H) and 2.21-2.13 (m, 2H).

### Step F: 7-fluoro-5-(pyridin-4-yl)-2,3-dihydro-1H-inden-4-amine

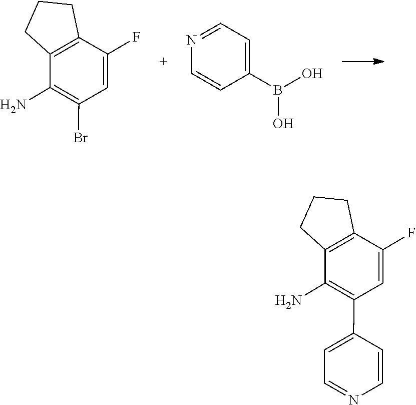 [SMILES: Nc1c(Br)cc(F)c2c1CCC2 ; OB(O)c1ccncc1 ; Nc1c(-c2ccncc2)cc(F)c2c1CCC2]

To a mixture of 5-bromo-7-fluoro-2,3-dihydro-1H-inden-4-amine (5 g, 21.73 mmol, 1 eq) and pyridin-4-ylboronic acid (2.94 g, 23.91 mmol, 1.1 eq) in dioxane (80 mL) and H₂O (10 mL) was added K₂CO₃ (7.51 g, 54.33 mmol, 2.5 eq) and Pd(dppf)Cl₂ (795 mg, 1.09 mmol, 0.05 eq) in one portion under N₂. Then the reaction mixture was heated to 80° C. and stirred for 12 hours. The mixture was quenched with water (100 mL) and extracted with EtOAc (3×10 mL). The combined organic phases were washed with brine (100 mL), dried over anhydrous Na₂SO₄, filtered and concentrated in vacuum. The residue was purified by column chromatography (SiO₂, petroleum ether:ethyl acetate 10:1 to 1:3) to give the title compound (4.5 g, 90% yield) as a brown solid.

¹H NMR (400 MHz, CDCl₃): δ 8.68 (dd, 2H), 7.40 (dd, 2H), 6.72 (d, 1H), 3.56 (br s, 2H), 3.01 (t, 2H), 2.80 (t, 2H) and 2.26-2.18 (m, 2H).

LCMS: m/z 229.1 (M+H)⁺ (ES⁺).

### Intermediate R8: 5-((7-fluoro-5-(pyridin-4-yl)-2,3-dihydro-1H-inden-4-yl)amino)-4H-1,2,4-triazole-3-thiol

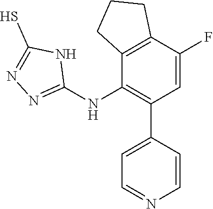 [SMILES: Fc1cc(-c2ccncc2)c(Nc2nnc(S)[nH]2)c2c1CCC2]

### Step A: 4-(7-fluoro-4-isothiocyanato-2,3-dihydro-1H-inden-5-yl)pyridine

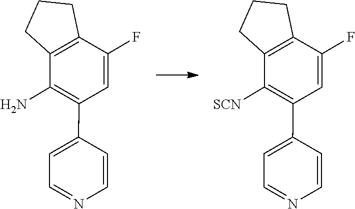

A solution of 7-fluoro-5-(pyridin-4-yl)-2,3-dihydro-1H-inden-4-amine (Intermediate R⁷) (6 g, 26.29 mmol, 1 eq) in DCM (60 mL) was added into another solution of thiocarbonyl dichloride (4-53 g, 39.43 mmol, 1.5 eq) and CaCO₃ (4.21 g, 42.06 mmol, 1.6 eq) in DCM (120 mL) and H₂O (60 mL). The reaction mixture was stirred at 25° C. for 12 hours. The mixture was quenched with water (100 mL) and extracted with DCM (2×100 mL). The combined organic phases were washed with brine (100 mL), dried over anhydrous Na₂SO₄, filtered and concentrated in vacuum. The residue was purified by column chromatography (SiO₂, petroleum ether:ethyl acetate 50:1 to 10:1) to give the title compound (3 g, 42% yield) as a yellow solid.

¹H NMR (400 MHz, CDCl₃): δ 8.73 (d, 2H), 7.41 (d, 2H), 6.91 (d, 1H), 3.14 (t, 2H), 3.05 (t, 2H) and 2.29-2.21 (m, 2H).

### Step B: 1-carbamothioyl-3-[7-fluoro-5-(4-pyridyl)indan-4-yl]thiourea

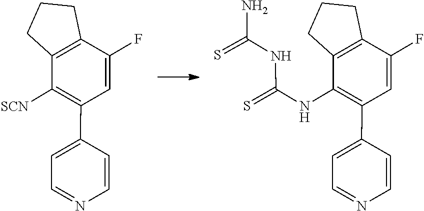

To a mixture of thiourea (1.69 g, 22.20 mmol, 2 eq) in THF (30 mL) was added t-BuONa (1.60 g, 16.65 mmol, 1.5 eq) in one portion under N₂. The reaction mixture was stirred at 25° C. for 0.5 hour. Then the solution of 4-(7-fluoro-4-isothiocyanato-2,3-dihydro-1H-inden-5-yl)pyridine (3 g, 11.10 mmol, 1 eq) in THF (15 mL) was added to the above mixture and the resulting reaction mixture was stirred at 25° C. for 12 hours. The mixture was quenched with citric acid (150 mL) and extracted with EtOAc (2×150 mL). The combined organic phases were dried over anhydrous Na₂SO₄, filtered and the filtrate was concentrated in vacuum to give the title compound (8.3 g, crude) as a yellow oil.

¹H NMR (400 MHz, DMSO-d₆): δ 8.61 (dd, 2H), 7.41 (dd, 2H), 7.14 (d, 1H), 2.99-2.77 (m, 4H) and 2.25-2.15 (m, 2H). Four exchangeable protons not observed.

LCMS: m/z 347.1 (M+H)⁺ (ES⁺).

### Step C: ethyl N′-(7-fluoro-5-(pyridin-4-yl)-2,3-dihydro-1H-inden-4-yl)carbamothioyl-carbamimidothioate and ethyl N′-carbamothioyl-N-(7-fluoro-5-(pyridin-4-yl)-2,3-dihydro-1H-inden-4-yl)carbamimidothioate

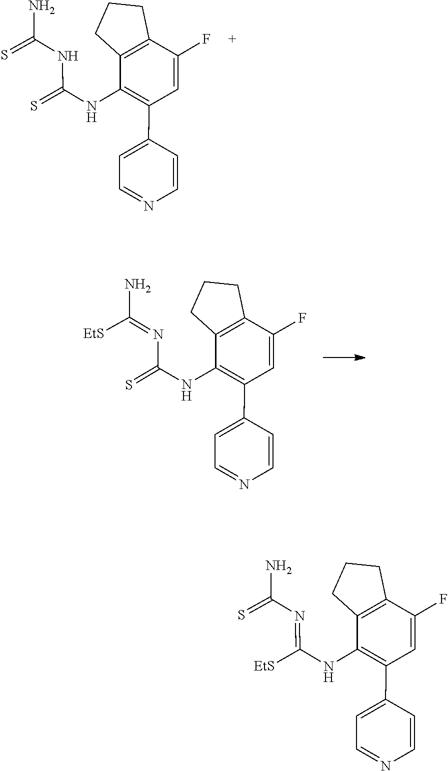

To a mixture of 1-carbamothioyl-3-[7-fluoro-5-(4-pyridyl)indan-4-yl]thiourea (7.8 g, 13.73 mmol, 1 eq) and TEA (2.08 g, 20.60 mmol, 1.5 eq) in DMF (80 mL) was added EtI (2.03 g, 13.05 mmol, 0.95 eq) in one portion. Then the reaction mixture was stirred at 25° C. for 0.5 hours. The reaction mixture was quenched with water (150 mL) and extracted with EtOAc (3×150 mL). The combined organic phases were washed with brine (150 mL), dried over anhydrous Na₂SO₄, filtered and concentrated in vacuum. The residue was purified by column chromatography (SiO₂, petroleum ether:ethyl acetate 10:1 to 0:1) to give the title compound (2.4 g, mixture) as a yellow solid.

¹H NMR (400 MHz, DMSO-d₆): δ 8.60-8.54 (m, 2H), 7.48-7.36 (m, 2H), 7.17-7.03 (m, 1H), 3.01-2.98 (m, 4H), 2.99-2.95 (m, 2H), 2.20-2.10 (m, 2H) and 1.08-0.97 (m, 3H). Three exchangeable protons not observed.

LCMS: m/z 375.1 (M+H)⁺ (ES⁺).

### Step D: 5-((7-fluoro-5-(pyridin-4-yl)-2,3-dihydro-1H-inden-4-yl)amino)-4H-1,2,4-triazole-3-thiol

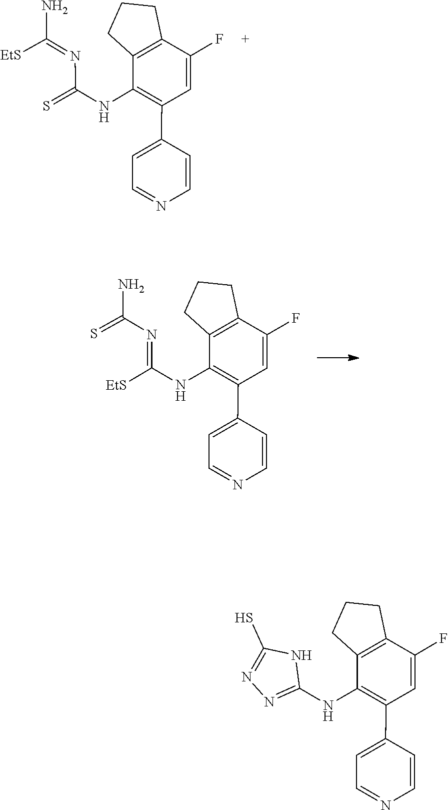

To a mixture of ethyl N′-carbamothioyl-N-(7-fluoro-5-(pyridin-4-yl)-2,3-dihydro-1H-inden-4-yl)carbamimidothioate and ethyl N′-(7-fluoro-5-(pyridin-4-yl)-2,3-dihydro-1H-inden-4-yl)carbamothioylcarbamimidothioate (0.9 g, 865.16 μmol, 1 eq) in EtOH (10 mL) and AcOH (1 mL) at 25° C. was added NH₂NH₂.H₂O (1.33 g, 25.95 mmol, 98 wt. % in aqueous solution, 30 eq) in one portion under N₂. Then the reaction mixture was heated to 80° C. and stirred for 1 hour. The mixture was directly purified by reversed phase flash chromatography (0.1% NH₃.H₂O-MeCN) to give the title compound (150 mg, 11.7% yield over three steps) as a yellow solid.

¹H NMR (400 MHz, DMSO-d₆): δ 8.56 (d, 2H), 8.10 (s, 1H), 7.36 (d, 2H), 7.04 (d, 1H), 2.96 (t, 2H), 2.76 (t, 2H) and 2.09-2.06 (m, 2H). Two exchangeable protons not observed.

LCMS: m/z 328.1 (M+H)⁺ (ES⁺).

### Intermediate R9: 4-fluoro-2-isopropyl-6-(pyridin-3-yl)aniline

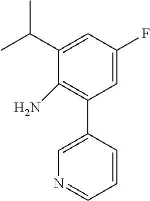 [SMILES: CC(C)c1cc(F)cc(-c2cccnc2)c1N]

### Step A: 4-fluoro-2-(prop-1-en-2-yl)aniline

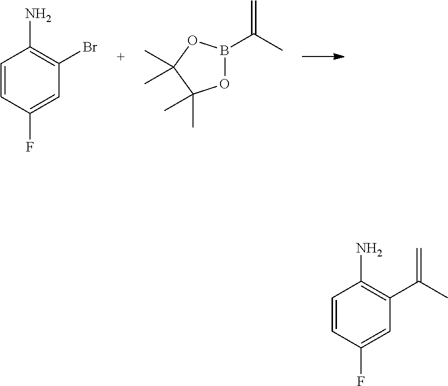

To a mixture of 2-bromo-4-fluoroaniline (39 g, 205.25 mmol, 1 eq) and 4,4,5,5-tetramethyl-2-(prop-1-en-2-yl)-1,3,2-dioxaborolane (36.21 g, 215.51 mmol, 1.05 eq) and K₂CO₃ (70.92 g, 513.12 mmol, 2.5 eq) in dioxane (200 mL) and H₂O (40 mL) was added Pd(dppf)Cl₂ (7.51 g, 10.26 mmol, 0.05 eq). Then the reaction mixture was stirred at 80° C. for 5 hours under N₂ atmosphere. The reaction mixture was quenched by addition of H₂O (600 mL) and extracted with EtOAc (2×500 mL). The combined organic layers were washed with brine (2×600 mL), dried over anhydrous Na₂SO₄, filtered and concentrated under reduced pressure. The residue was purified by column chromatography (SiO₂, petroleum ether:ethyl acetate 1:0 to 100:1) to give the title compound (27 g, 89% purity on LCMS, 77.44% yield) as a yellow oil.

¹H NMR (400 MHz, CDCl₃): δ 6.81-6.76 (m, 2H), 6.66-6.62 (m, 1H), 5.38 (s, 1H), 5.08 (s, 1H), 3.69 (br s, 2H) and 1.25 (s, 3H).

LCMS: m/z 152.2 (M+H)⁺ (ES⁺).

### Step B: 4-fluoro-2-isopropylaniline

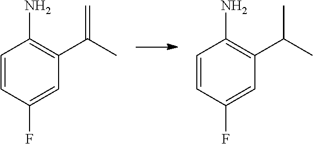

To a solution of 4-fluoro-2-(prop-1-en-2-yl)aniline (21 g, 138.91 mmol, 1 eq) in MeOH (300 mL) was added Pd/C (2.1 g, 10 wt. % loading on activated carbon) under N₂ atmosphere. The reaction mixture was degassed in vacuum and purged with H₂ several times. The reaction mixture was stirred at 25° C. for 12 hours under H₂ (50 psi). The reaction mixture was filtered and the filtrate was concentrated in vacuum to give the title compound (20 g, crude) as a yellow oil.

¹H NMR (400 MHz, CDCl₃): δ 6.86 (dd, 1H), 6.75-6.72 (m, 1H), 6.63-6.61 (m, 1H), 3.50 (br s, 2H), 2.95-2.84 (m, 1H) and 1.25 (d, 6H).

LCMS: m/z 154.2 (M+H)⁺ (ES⁺).

### Step C: 2-bromo-4-fluoro-6-isopropylaniline

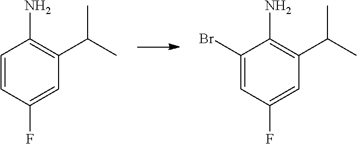

To a solution of 4-fluoro-2-isopropylaniline (20 g, 130.55 mmol, 1 eq) in toluene (250 mL) was added NBS (23.24 g, 130.55 mmol, 1 eq) at 25° C. The reaction mixture was stirred at 25° C. for 10 minutes. The reaction mixture was poured into H₂O (300 mL) and extracted with EtOAc (2×250 mL). The organic phases were washed with brine (2×400 mL), dried over anhydrous Na₂SO₄, filtered and concentrated in vacuum. The residue was purified by column chromatography (SiO₂, only eluting with petroleum ether) to give the title compound (30 g, 93.0% yield over two steps) as a black brown oil.

¹H NMR (400 MHz, CDCl₃): δ 6.99 (dd, 1H), 6.78 (dd, 1H), 3.91 (br s, 2H), 2.88-2.71 (m, 1H) and 1.17 (d, 6H).

LCMS: m/z 232.1 (M+H)⁺ (ES⁺).

### Step D: 4-fluoro-2-isopropyl-6-(pyridin-3-yl)aniline

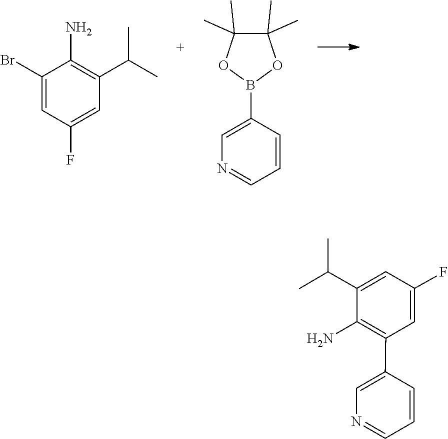

To a solution of 2-bromo-4-fluoro-6-isopropylaniline (21 g, 90.48 mmol, 1 eq) in dioxane (450 mL) and H₂O (90 mL) was added 3-(4,4,5,5-tetramethyl-1,3,2-dioxaborolan-2-yl)pyridine (22.26 g, 108.58 mmol, 1.2 eq) and Na₂CO₃ (23.98 g, 226.20 mmol, 2.5 eq). Then the reaction mixture was purged with N₂ three times. Then Pd(dppf)Cl₂ (5.10 g, 6.97 mmol, 0.077 eq) was added to the above mixture under N₂ atmosphere. The resulting mixture was heated to 80° C. and stirred for 2 hours. The reaction mixture was quenched by addition of H₂O (800 mL) and extracted with EtOAc (2×600 mL). The combined organic layers were washed with brine (2×800 mL), dried over anhydrous Na₂SO₄, filtered and concentrated under reduced pressure. The residue was purified by column chromatography (SiO₂, petroleum ether:ethyl acetate 50:1 to 1:1) and then triturated with hexane (40 mL) to give the title compound (17 g, 81.59% yield) as a grey solid.

¹H NMR (400 MHz, CDCl₃): δ 8.70 (d, 1H), 8.63 (dd, 1H), 7.79 (dd, 1H), 7.41-7.38 (m, 1H), 6.94 (dd, 1H), 6.71 (dd, 1H), 3.57 (s, 2H), 2.97-2.88 (m, 1H) and 1.30 (d, 6H).

LCMS: m/z 231.2 (M+H)⁺ (ES⁺).

### Intermediate R10:5-((4-fluoro-2-isopropyl-6-(pyridin-3-yl)phenyl)amino)-4H-1,2,4-triazole-3-thiol

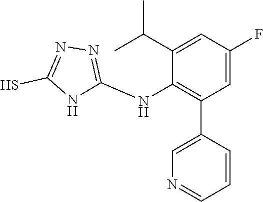 [SMILES: CC(C)c1cc(F)cc(-c2cccnc2)c1Nc1nnc(S)[nH]1]

### Step A: 3-(5-fluoro-3-isopropyl-2-isothiocyanatophenyl)pyridine

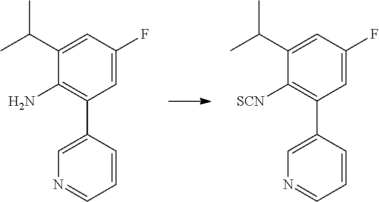

A solution of 4-fluoro-2-isopropyl-6-(pyridin-3-yl)aniline (Intermediate R9) (1 g, 4.34 mmol, 1 eq) in EtOH (10 mL) was treated with CS₂ (1.32 g, 17.37 mmol, 4 eq) followed by TEA (439 mg, 4.34 mmol, 1 eq). After stirring at 20° C. for 1 hour, Boc₂O (947 mg, 4.34 mmol, 1 eq) and DMAP (15 mg, 130.28 μmol, 0.03 eq) were added to the reaction mixture at 0° C. Then the reaction mixture was stirred at 20° C. for 15 hours. The reaction mixture was quenched by addition of EtOAc (10 mL) and H₂O (10 mL). The mixture was extracted with EtOAc (3×10 mL). The combined organic layers were dried over anhydrous Na₂SO₄, filtered and concentrated under reduced pressure. The residue was purified by column chromatography (SiO₂, petroleum ether:ethyl acetate 20:1 to 10:1) to give the title compound (0.35 g, 29.59% yield) as a yellow solid.

¹H NMR (400 MHz, CDCl₃): δ 8.69 (d, 2H), 7.80 (dd, 1H), 7.45 (dd, 1H), 7.05 (dd, 1H), 6.94 (dd, 1H), 3.37-3.27 (m, 1H) and 1.31 (d, 6H).

LCMS: m/z 273.3 (M+H)⁺ (ES⁺).

### Step B: 1-carbamothioyl-3-[4-fluoro-2-isopropyl-6-(3-pyridyl)phenyl]thiourea

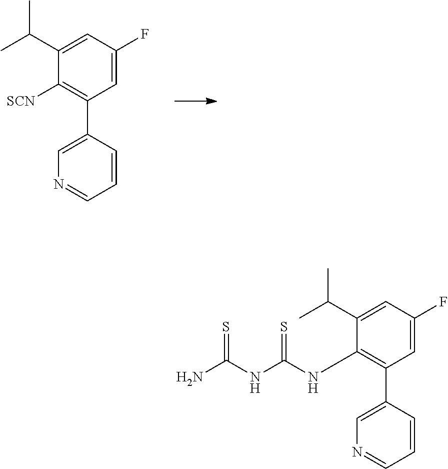

A solution of thiourea (380 mg, 4.99 mmol, 2 eq) and t-BuONa (359 mg, 3.75 mmol, 1.5 eq) in THF (5 mL) was stirred at 20° C. for 30 minutes. Then to the mixture was added 3-(5-fluoro-3-isopropyl-2-isothiocyanatophenyl)pyridine (0.68 g, 2.50 mmol, 1 eq). The resulting mixture was stirred at 20° C. for 16 hours. The reaction mixture was quenched by addition of H₂O (15 mL), and then adjusted to pH 6 with citric acid, and extracted with EtOAc (2×30 mL). The combined organic layers were washed with brine (2×30 mL), dried over anhydrous Na₂SO₄, filtered and concentrated under reduced pressure. The residue was purified by column chromatography (SiO₂, petroleum ether:ethyl acetate 2.5:1 to 1.2:1) to give the title compound (0.9 g, 96.20% yield, 93% purity on LCMS) as a yellow solid.

¹H NMR (400 MHz, DMSO-d₆): 12.19 (s, 2H), 10.74 (s, 1H), 9.20 (s, 1H), 8.59 (dd, 1H), 8.55 (dd, 1H), 7.81 (dd, 1H), 7.32 (dd, 1H), 7.29 (dd, 1H), 7.16 (dd, 1H), 3.09-2.97 (m, 1H) and 1.17 (d, 6H).

LCMS: m/z 349.1 (M+H)⁺ (ES⁺).

### Step C: ethyl N′-(4-fluoro-2-isopropyl-6-(pyridin-3-yl)phenyl)carbamothioyl-carbamimidothioate and ethyl N′-carbamothioyl-N-(4-fluoro-2-isopropyl-6-(pyridin-3-yl)phenyl)carbamimidothioate

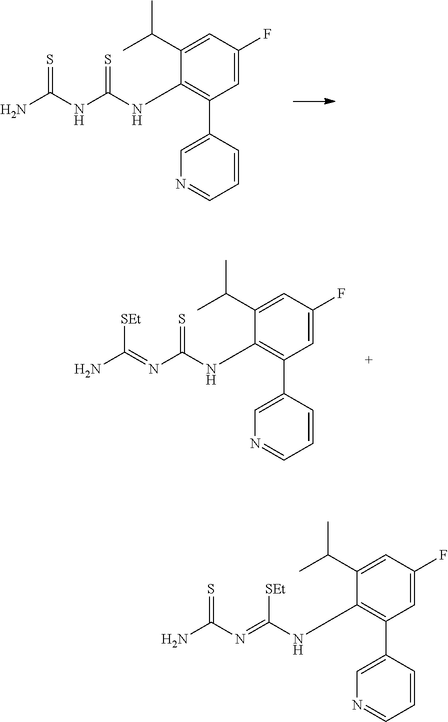

To a solution of 1-carbamothioyl-3-[4-fluoro-2-isopropyl-6-(3-pyridyl)phenyl]thiourea (900 mg, 2.58 mmol, 1 eq) and TEA (522 mg, 5.17 mmol, 2 eq) in DMF (8 mL) was added EtI (281 mg, 1.81 mmol, 0.7 eq). Then the solution was stirred at 25° C. for 30 minutes. The reaction mixture was quenched with H₂O (15 mL) and extracted with EtOAc (3×30 mL). The organic layers were washed with brine (2×80 mL), dried over anhydrous Na₂SO₄, filtered and concentrated in vacuum. The residue was purified by column chromatography (SiO₂, petroleum ether:ethyl acetate 7:1 to 5:1) to give the title compound (1 g, mixture) as a yellow oil.

LCMS: m/z 377.2 (M+H)⁺ (ES⁺).

### Step D: 5-((4-fluoro-2-isopropyl-6-(pyridin-3-yl)phenyl)amino)-4H-1,2,4-triazole-3-thiol

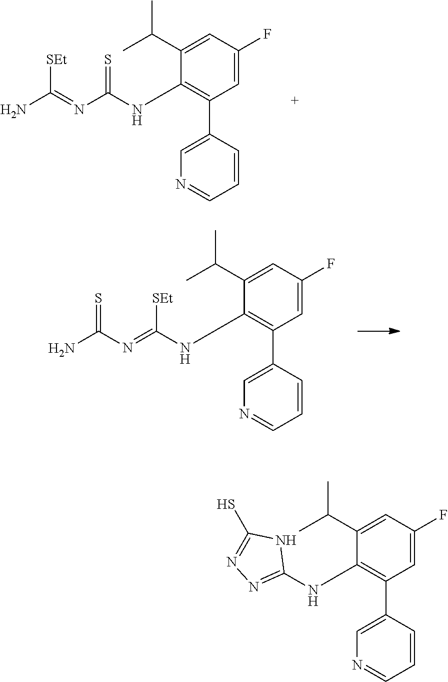

To a solution of ethyl N′-(4-fluoro-2-isopropyl-6-(pyridin-3-yl)phenyl)carbamothioyl-carbamimidothioate and ethyl N′-carbamothioyl-N-(4-fluoro-2-isopropyl-6-(pyridin-3-yl)phenyl)carbamimidothioate (1.1 g, mixture, 1 eq) in EtOH (5 mL) and AcOH (0.5 mL) was added NH₂NH₂.H₂O (2.34 g, 46.74 mmol, 98 wt. % in aqueous solution, 40 eq). Then the reaction mixture was stirred at 80° C. for 0.5 hour. The mixture was concentrated in vacuum. The residue was purified by reversed phase flash chromatography (0.1% NH₃.H₂O-MeCN) to give the title compound (70 mg, 10.7% yield over two steps) as a yellow solid.

¹H NMR (400 MHz, DMSO-d₆): δ 8.57 (d, 1H), 8.49 (dd, 1H), 8.48 (br s, 1H), 7.81 (dd, 1H), 7.35 (dd, 1H), 7.23 (dd, 1H), 7.07 (dd, 1H), 3.25-3.17 (m, 1H) and 1.15 (d, 6H). Two exchangeable protons not observed.

LCMS: m/z 330.1 (M+H)⁺ (ES⁺).

### Intermediate R1:4-fluoro-2-isopropyl-6-(pyridin-4-yl)aniline

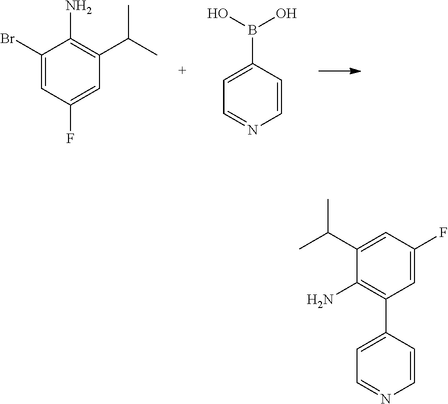 [SMILES: CC(C)c1cc(F)cc(Br)c1N ; OB(O)c1ccncc1 ; CC(C)c1cc(F)cc(-c2ccncc2)c1N]

To a mixture of 2-bromo-4-fluoro-6-isopropyl-aniline (Intermediate R9, Step C) (23.5 g, 101.25 mmol, 1 eq) and pyridin-4-ylboronic acid (13.07 g, 106.32 mmol, 1.05 eq) in dioxane (250 mL) and H₂O (50 mL) were added K₂CO₃ (34.98 g, 253.13 mmol, 2.5 eq) and Pd(dppf)Cl₂ (7.41 g, 10.13 mmol, 0.1 eq) in one portion under N₂. Then the reaction mixture was heated to 100° C. and stirred for 5 hours. The mixture was filtered and the filtrate was concentrated in vacuum. The residue was purified by silica gel column chromatography (SiO₂, petroleum ether:ethyl acetate 50:1 to 1:2 and then ethyl acetate:ethanol 10:1, 0.1% NH₃.H₂O) to give the title compound (20.43 g, 87.6% yield) as a brown solid.

¹H NMR (400 MHz, CDCl₃) δ 8.70 (d, 2H), 7.40 (d, 2H), 6.95 (dd, 1H), 6.72 (dd, 1H), 3.63 (br s, 2H), 2.96-2.89 (m, 1H), 1.29 (d, 6H).

LCMS: m/z 231.2 (M+H)⁺ (ES⁺).

### Intermediate R12:5-(4-fluoro-2-isopropyl-6-(pyridin-4-yl)benzyl)-4H-1,2,4-triazole-3-thiol

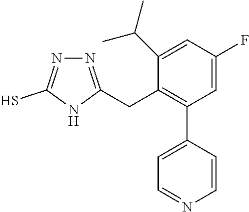 [SMILES: CC(C)c1cc(F)cc(-c2ccncc2)c1Cc1nnc(S)[nH]1]

### Step A: 4-(2-bromo-5-fluoro-3-isopropylphenyl)pyridine

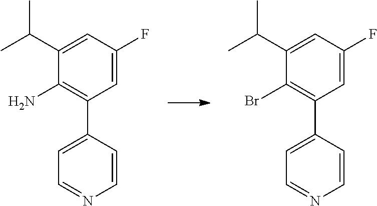 [SMILES: CC(C)c1cc(F)cc(-c2ccncc2)c1N ; CC(C)c1cc(F)cc(-c2ccncc2)c1Br]

To a mixture of 4-fluoro-2-isopropyl-6-(pyridin-4-yl)aniline (Intermediate R11) (19.3 g, 83.81 mmol, 1 eq) in MeCN (200 mL) was added dropwise isopentyl nitrite (11.78 g, 100.57 mmol, 1.2 eq) at ° C. Then the mixture was stirred at ° C. for 0.5 hour. Finally CuBr (18.03 g, 125.72 mmol, 1.5 eq) was added to the mixture and the reaction mixture was heated to 70° C. and stirred for 3 hours. The mixture was filtered and the filtrate was concentrated in vacuum. The residue was purified by silica gel column chromatography (SiO₂, petroleum ether:ethyl acetate, 20:1 to 1:1) to give the title compound (3.3 g, 13.3% yield, 100% purity on LCMS) as a brown solid.

¹H NMR (400 MHz, CDCl₃) δ 8.74-8.71 (m, 2H), 7.40-7.28 (m, 2H), 7.08 (dd, 1H), 6.86 (dd, 1H), 3.53-3.48 (m, 1H), 1.27 (d, 6H).

LCMS: m/z 294.0 (M+H)⁺ (ES⁺).

### Step B: tert-butyl 2-(4-fluoro-2-isopropyl-6-(pyridin-4-yl)phenyl)acetate

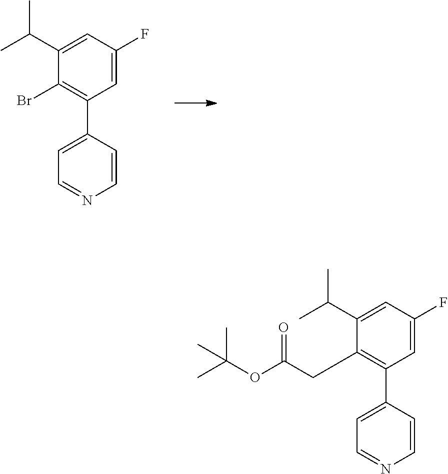 [SMILES: CC(C)c1cc(F)cc(-c2ccncc2)c1Br ; CC(C)c1cc(F)cc(-c2ccncc2)c1CC(=O)OC(C)(C)C]

To a mixture of 4-(2-bromo-5-fluoro-3-isopropyl-phenyl)pyridine (2.8 g, 9.52 mmol, 1 eq) and XPhos (453 mg, 951.86 μmol, 0.1 eq) and Pd₂(dba)₃ (435 mg, 475.93 μmol, 0.05 eq) in THF (10 mL) was added (2-(tert-butoxy)-2-oxoethyl) zinc (II) bromide (Intermediate R2, Step D) (0.78 M, 36.61 mL, 3 eq) at 25° C. under N₂. The mixture was stirred at 70° C. for 2 hours under N₂. The mixture was quenched with saturated aqueous NH₄Cl solution (50 mL) and extracted with EtOAc (3×50 mL). The combined organic phases were washed with brine (50 mL), dried over anhydrous Na₂SO₄, filtered and concentrated in vacuum. The residue was purified by silica gel column chromatography (SiO₂, petroleum ether:ethyl acetate, 50:1 to 10:1) to give the title compound (3.39 g, over two steps yield: 87.5%, 81% purity on LCMS) as a yellow solid.

¹H NMR (400 MHz, DMSO-d₆) δ 8.71-8.60 (m, 2H), 7.40-7.30 (m, 2H), 7.26 (dd, 1H), 6.93 (dd, 1H), 3.46 (s, 2H), 3.07-3.01 (m, 1H), 1.35 (s, 9H), 1.18 (d, 6H).

LCMS: m/z 330.2 (M+H)⁺ (ES⁺).

### Step C: 2-(4-fluoro-2-isopropyl-6-(pyridin-4-yl)phenyl)acetic acid

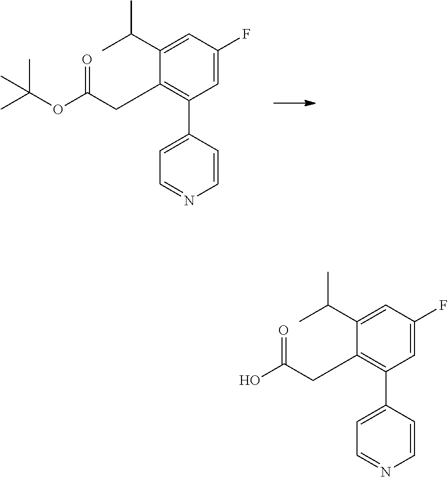 [SMILES: CC(C)c1cc(F)cc(-c2ccncc2)c1CC(=O)OC(C)(C)C ; CC(C)c1cc(F)cc(-c2ccncc2)c1CC(=O)O]

To a mixture of tert-butyl 2-(4-fluoro-2-isopropyl-6-(pyridin-4-yl)phenyl)acetate (4 g, 9.84 mmol, 1 eq) in DCM (20 mL) was added TFA (30.80 g, 270.12 mmol, 27.46 eq) in one portion at 25° C. Then the reaction mixture was stirred at 25° C. for 12 hours. The mixture was concentrated in vacuum. The residue was diluted with water (20 mL) and stirred at 25° C. for 0.5 hour. Then the mixture was filtered and the filter cake was dried in vacuum to give the title compound (2.6 g, 96.7% yield) as a yellow solid.

¹H NMR (400 MHz, DMSO-d₆) δ 8.80 (d, 2H), 7.60 (d, 2H), 7.30 (dd, 1H), 6.99 (dd, 1H), 3.49 (s, 2H), 3.08-3.05 (m, 1H), 1.23 (d, 6H). One exchangeable proton not observed.

LCMS: m/z 273.9 (M+H)⁺ (ES⁺).

### Step D: 2-(4-fluoro-2-isopropyl-6-(pyridin-4-yl)phenyl)acetyl chloride

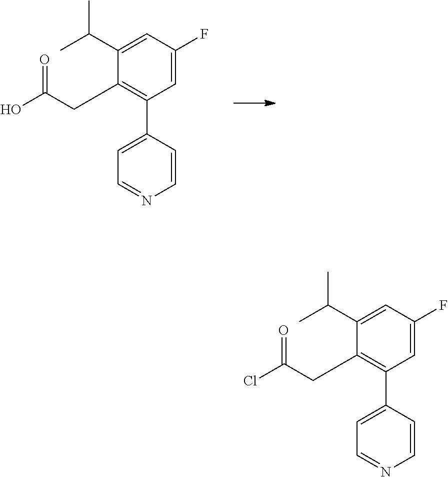 [SMILES: CC(C)c1cc(F)cc(-c2ccncc2)c1CC(=O)O ; CC(C)c1cc(F)cc(-c2ccncc2)c1CC(=O)Cl]

A mixture of 2-(4-fluoro-2-isopropyl-6-(pyridin-4-yl)phenyl)acetic acid (1 g, 3.66 mmol, 1 eq) in SOCl₂ (10 mL) was heated to 50° C. and stirred for 1 hour. The mixture was concentrated in vacuum to give the title compound (1.07 g, crude) as a brown solid, which was used directly in the next step without further purification.

### Step E: 2-(2-(4-fluoro-2-isopropyl-6-(pyridin-4-yl)phenyl)acetyl)hydrazine-carbothioamide

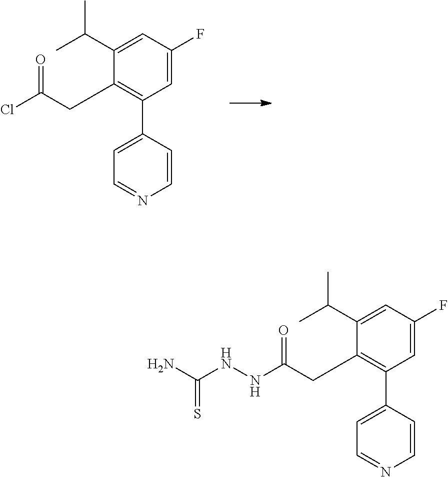 [SMILES: CC(C)c1cc(F)cc(-c2ccncc2)c1CC(=O)Cl ; CC(C)c1cc(F)cc(-c2ccncc2)c1CC(=O)NNC(N)=S]

To a solution of hydrazinecarbothioamide (0.318 g, 3.49 mmol, 1 eq) in pyridine (10 mL) was added a solution of 2-(4-fluoro-2-isopropyl-6-(pyridin-4-yl)phenyl)acetyl chloride (1.07 g, 3.66 mmol, 1.05 eq) in toluene (10 mL) dropwise at 0° C. After addition, the mixture was warmed to 25° C. and stirred for 12 hours under N₂. The mixture was concentrated in vacuum. The residue was purified by reversed phase flash chromatography (water (0.1% TFA v/v)-MeCN) to give the title compound (850 mg, yield over two steps: 66.9% yield, 75.88% purity on LCMS) as a brown solid.

¹H NMR (400 MHz, DMSO-d₆) δ 9.77 (br s, 1H), 9.32 (br s, 1H), 8.76 (d, 2H), 7.90 (br s, 1H), 7.59-7.56 (m, 2H), 7.55 (br s, 1H), 7.28 (dd, 1H), 6.68-6.96 (m, 1H), 3.60 (s, 2H), 3.16-3.13 (m, 1H) and 1.18 (dd, 6H).

LCMS: m/z 347.1 (M+H)⁺ (ES⁺).

### Step F: 5-(4-fluoro-2-isopropyl-6-(pyridin-4-yl)benzyl)-4H-1,2,4-triazole-3-thiol

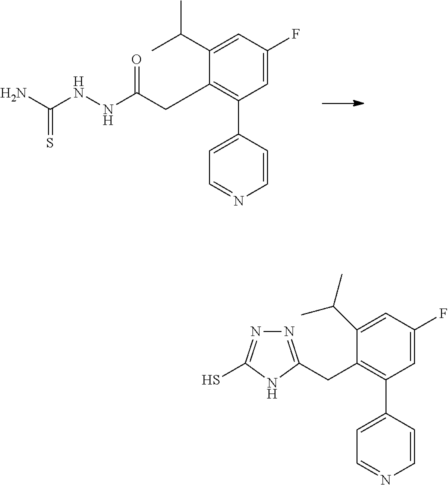 [SMILES: CC(C)c1cc(F)cc(-c2ccncc2)c1CC(=O)NNC(N)=S ; CC(C)c1cc(F)cc(-c2ccncc2)c1Cc1nnc(S)[nH]1]

To a mixture of 2-(2-(4-fluoro-2-isopropyl-6-(pyridin-4-yl)phenyl)acetyl)hydrazine-carbothioamide (850 mg, 1.86 mmol, 1 eq) in H₂O (30 mL) was added NaOH (894 mg, 22.34 mmol, 12 eq) in one portion. Then the reaction mixture was heated to 100° C. and stirred for 3 hours. The mixture was cooled to 20° C. and adjusted to pH=6 with 1 N HCl aqueous solution. Then the suspension mixture was filtered and the filter cake was dried in vacuum to give the title compound (400 mg, 5037% yield, 77.0% purity on LCMS) as a yellow solid.

¹H NMR (400 MHz, DMSO-d₆) δ 13.23 (s, 1H), 13.07 (br s, 1H), 8.62 (d, 2H), 7.31-7.26 (m, 3H), 6.95 (dd, 1H), 3.73 (s, 2H), 3.97-2.93 (m, 1H) and 1.12 (d, 6H).

LCMS: m/z 329.2 (M+H)⁺ (ES⁺).

### Intermediate R13: 5-(4-fluoro-2-isopropyl-6-(pyridin-4-yl)benzyl)-4H-1,2,4-triazole-3-sulfonyl chloride

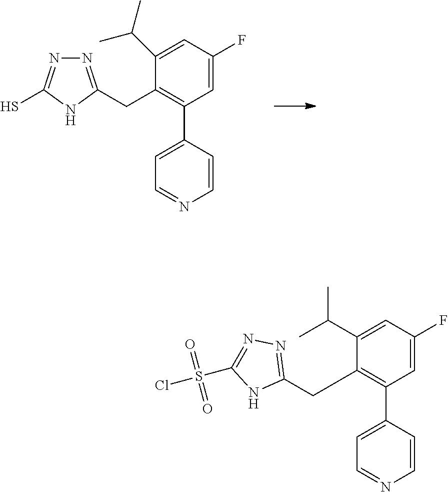 [SMILES: CC(C)c1cc(F)cc(-c2ccncc2)c1Cc1nnc(S)[nH]1 ; CC(C)c1cc(F)cc(-c2ccncc2)c1Cc1nnc(S(=O)(=O)Cl)[nH]1]

To a solution of 5-(4-fluoro-2-isopropyl-6-(pyridin-4-yl)benzyl)-4H-1,2,4-triazole-3-thiol (100 mg, 304.50 μmol, 1 eq) in AcOH (3 mL) and H₂O (0.6 mL) was added NCS (122 mg, 913.50 μmol, 3 eq) in one portion at 25° C. Then the reaction mixture was stirred for 12 hours. The mixture was quenched with water (20 mL) and extracted with DCM (3×20 mL). The combined organic phases were washed with brine (20 mL), dried over anhydrous Na₂SO₄, filtered and concentrated in vacuum to give the title compound (0.12 g, crude) as a yellow oil, which was used directly in the next step without further purification.

### Intermediate R14: 4-fluoro-2-isopropyl-6-(2-methoxypyridin-4-yl)aniline

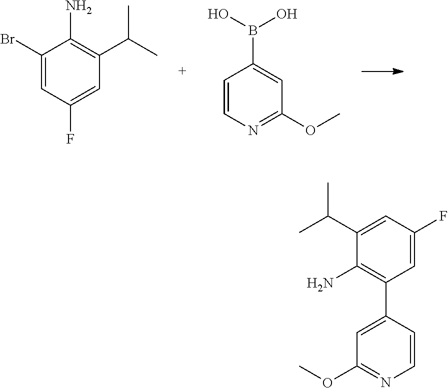 [SMILES: CC(C)c1cc(F)cc(Br)c1N ; COc1cc(B(O)O)ccn1 ; COc1cc(-c2cc(F)cc(C(C)C)c2N)ccn1]

To a solution of 2-bromo-4-fluoro-6-isopropylaniline (Intermediate R9, Step C) (12 g, 51.70 mmol, 1 eq) in dioxane (240 mL) and H₂O (48 mL) was added (2-methoxy-pyridin-4-yl)boronic acid (9.5 g, 62.04 mmol, 1.2 eq) and Na₂CO₃ (13.7 g, 129.26 mmol, 2.5 eq). Then the reaction mixture was purged with N₂ three times. Then to the mixture was added Pd(dppf)Cl₂ (3.78 g, 5.17 mmol, 0.1 eq) under N₂. The resulting mixture was heated at 80° C. and stirred for 2 hours. The reaction mixture was quenched with H₂O (800 mL) and extracted with EtOAc (2×600 mL). The combined organic layers were washed with brine (2×800 mL), dried over anhydrous Na₂SO₄, filtered and concentrated under reduced pressure. The residue was purified by silica gel column chromatography (SiO₂, petroleum ether:ethyl acetate, 70:1 to 10:1), and then triturated with hexane (100 mL) to give the title compound (10.05 g, 71.7% yield, 96.0% purity on LCMS).

¹H NMR (400 MHz, CDCl₃) δ 8.24 (d, 1H), 6.97 (d, 1H), 6.93 (d, 1H), 6.83 (s, 1H), 6.73-6.70 (m, 1H), 3.99 (s, 3H), 3.66 (br s, 2H), 2.97-2.89 (m, 1H), 1.29 (dd, 6H).

LCMS: m/z 261.1 (M+H)⁺ (ES⁺).

### Intermediate R15: 5-(4-fluoro-2-isopropyl-6-(2-methoxypyridin-4-yl)benzyl)-4H-1,2,4-triazole-3-thiol

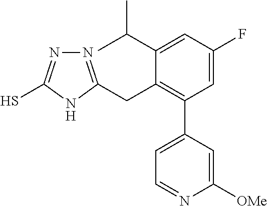 [SMILES: COc1cc(-c2cc(F)cc(C(C)C)c2Cc2nnc(S)[nH]2)ccn1]

### Step A: 4-(2-bromo-5-fluoro-3-isopropylphenyl)-2-methoxypyridine

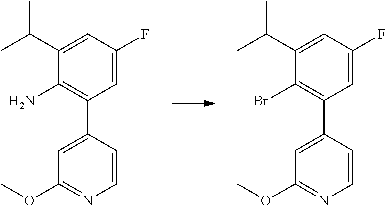 [SMILES: COc1cc(-c2cc(F)cc(C(C)C)c2N)ccn1 ; COc1cc(-c2cc(F)cc(C(C)C)c2Br)ccn1]

To a solution of 4-fluoro-2-isopropyl-6-(2-methoxypyridin-4-yl)aniline (Intermediate R14) (2.02 g, 7.76 mmol, 1 eq), CuBr (1.32 g, 9.31 mmol, 1.2 eq) and CuBr₂ (9 mg, 38.8 μmol, 0.005 eq) in MeCN (20 mL) was added isopentyl nitrite (1.17 g, 10.1 mmol, 1.3 eq) at ° C. Then the reaction mixture was stirred at 6° C. for 40 minutes. The reaction mixture was poured into H₂O (40 mL) and extracted with EtOAc (3×40 mL). The organic phases were washed with brine (2×50 mL), dried over anhydrous Na₂SO₄, filtered and concentrated in vacuum. The residue was purified by silica gel column chromatography (SiO₂, petroleum ether:ethyl acetate, 1:0 to 70:1) to give the title compound (1.7 g, 68.3% yield) as a red oil.

¹H NMR (400 MHz, CDCl₃): δ 8.22 (d, 1H), 7.06 (dd, 1H), 6.88 (dd, 1H), 6.84 (d, 1H), 6.73 (d, 1H), 4.00 (s, 3H), 3.53-3.46 (m, 1H), 1.28 (d, 6H).

LCMS: m/z 324.1 (M+H)⁺ (ES⁺).

### Step B: tert-butyl 2-(4-fluoro-2-isopropyl-6-(2-methoxypyridin-4-yl)phenyl)acetate

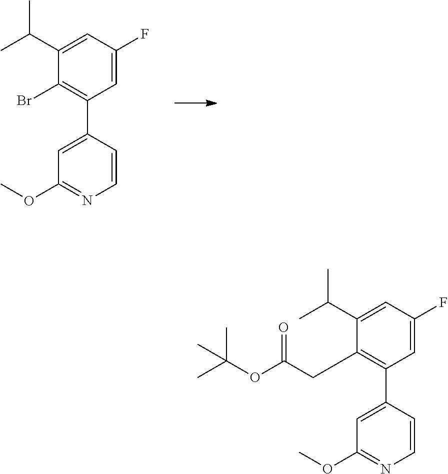 [SMILES: COc1cc(-c2cc(F)cc(C(C)C)c2Br)ccn1 ; COc1cc(-c2cc(F)cc(C(C)C)c2CC(=O)OC(C)(C)C)ccn1]

To a solution of 4-(2-bromo-5-fluoro-3-isopropylphenyl)-2-methoxypyridine (2.8 g, 8.64 mmol, 1 eq), Pd₂(dba)₃ (411 mg, 0.052 eq) and XPhos (412 mg, 0.1 eq) in THF (3 mL) was added a solution of (2-(tert-butoxy)-2-oxoethyl)zinc(II) bromide (Intermediate R2, Step D) in THF (0.5 M, 69 mL, 4 eq) under N₂ atmosphere. The reaction mixture was stirred at 70° C. for 14 hours. The mixture was poured into H₂O (150 mL) and extracted with EtOAc (3×150 mL). The organic phases were washed with brine (2×300 mL), dried over anhydrous Na₂SO₄, filtered and concentrated in vacuum. The residue was purified by silica gel column chromatography (SiO₂, petroleum ether:ethyl acetate, 1:0 to 70:1) to give the title compound (2.8 g, yield over two steps 90.2%) as a yellow oil.

¹H NMR (400 MHz, CDCl₃) δ 8.18 (d, 1H), 7.05 (d, 1H), 6.83 (dd, 1H), 6.76 (d, 1H), 6.69 (s, 1H), 3.98 (s, 3H), 3.46 (s, 2H), 3.13-3.03 (m, 1H), 1.42 (s, 9H), 1.25 (d, 6H).

LCMS: m/z 360.3 (M+H)⁺ (ES⁺).

### Step C: 2-(4-fluoro-2-isopropyl-6-(2-methoxypyridin-4-yl)phenyl)acetic acid

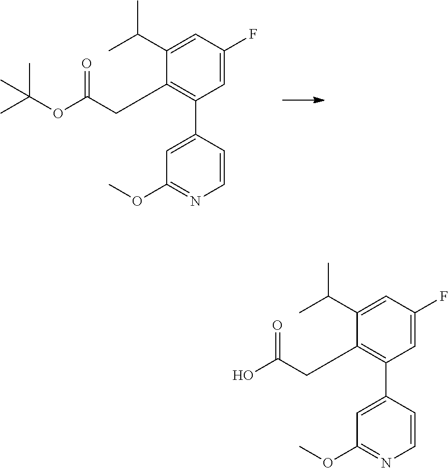 [SMILES: COc1cc(-c2cc(F)cc(C(C)C)c2CC(=O)OC(C)(C)C)ccn1 ; COc1cc(-c2cc(F)cc(C(C)C)c2CC(=O)O)ccn1]

To a solution of tert-butyl 2-(4-fluoro-2-isopropyl-6-(2-methoxypyridin-4-yl)phenyl)acetate (2.6 g, 7.23 mmol, 1 eq) in DCM (17 mL) was added TFA (26.69 g, 233.96 mmol, 32.36 eq). Then the mixture was stirred at 20° C. for 2 hours. The reaction solution was concentrated in vacuum. The residue was purified by reversed phase flash chromatography (water (0.1% TFA v/v)-MeCN) to give the title compound (2 g, 91.15% yield) as a white solid.

¹H NMR (400 MHz, DMSO-d₆) δ 8.22 (d, 1H), 7.24 (dd, 1H), 6.93-6.89 (m, 2H), 6.71 (s, 1H), 3.88 (s, 3H), 3.47 (s, 2H), 3.07-3.00 (m, 1H), 1.18 (d, 6H). One exchangeable proton not observed.

LCMS: m/z 304.3 (M+H)⁺ (ES⁺).

### Step D: 2-(4-fluoro-2-isopropyl-6-(2-methoxypyridin-4-yl)phenyl)acetyl chloride

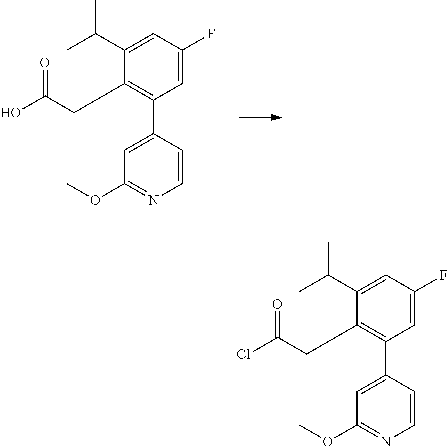 [SMILES: COc1cc(-c2cc(F)cc(C(C)C)c2CC(=O)O)ccn1 ; COc1cc(-c2cc(F)cc(C(C)C)c2CC(=O)Cl)ccn1]

A solution of 2-(4-fluoro-2-isopropyl-6-(2-methoxypyridin-4-yl)phenyl)acetic acid (5 g, 16.48 mmol, 1 eq) in SOCl₂ (82 g, 689.25 mmol, 41.81 eq) was stirred at 50° C. for 1 hour. The reaction mixture was concentrated in vacuum to give the title compound as a yellow solid (5.3 g, crude), which was used directly in the next step.

### Step E: 2-(2-(4-fluoro-2-isopropyl-6-(2-methoxypyridin-4-yl)phenyl)acetyl)hydrazine-carbothioamide

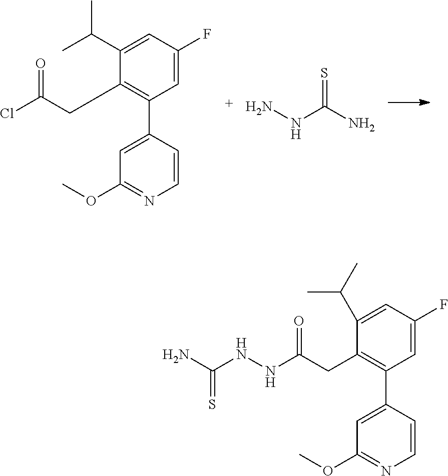 [SMILES: COc1cc(-c2cc(F)cc(C(C)C)c2CC(=O)Cl)ccn1 ; NNC(N)=S ; COc1cc(-c2cc(F)cc(C(C)C)c2CC(=O)NNC(N)=S)ccn1]

To a solution of hydrazinecarbothioamide (1.43 g, 15.69 mmol, 1 eq) in pyridine (25 mL) was added a solution of 2-(4-fluoro-2-isopropyl-6-(2-methoxypyridin-4-yl)-phenyl)acetyl chloride (5.3 g, 16.47 mmol, 1.05 eq) in toluene (25 mL) dropwise at 0° C. After addition, the mixture was warmed to 25° C. and stirred for 12 hours. The reaction mixture was concentrated in vacuum. The residue was purified by reversed phase flash chromatography (water (0.1% TFA v/v)-MeCN) to give the title compound (3.1 g, yield over 2 steps: 49.9%, 73.0% purity on LCMS) as a yellow solid.

¹H NMR (400 MHz, DMSO-d₆) δ 9.80 (br s, 1H), 9.33 (br s, 1H), 8.19 (d, 1H), 7.88 (br s, 1H), 7.41 (br s, 1H), 7.19 (dd, 1H), 6.90-6.86 (m, 2H), 6.77 (s, 1H), 3.87 (s, 3H), 3.44 (s, 2H), 3.15-3.05 (m, 1H), 1.14 (d, 6H).

LCMS: m/z 377.1 (M+H)⁺ (ES⁺).

### Step F: 5-(4-fluoro-2-isopropyl-6-(2-methoxypyridin-4-yl)benzyl)-4H-1,2,4-triazole-3-thiol

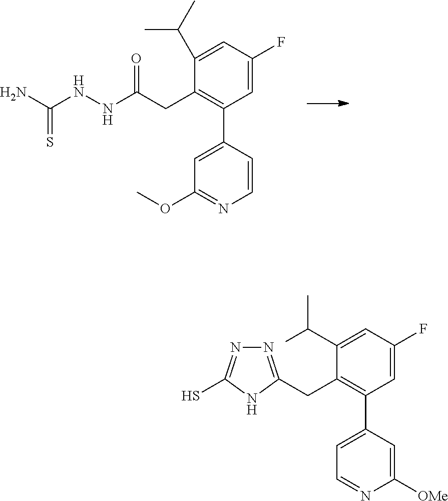 [SMILES: COc1cc(-c2cc(F)cc(C(C)C)c2CC(=O)NNC(N)=S)ccn1 ; COc1cc(-c2cc(F)cc(C(C)C)c2Cc2nnc(S)[nH]2)ccn1]

To a solution of 2-(2-(4-fluoro-2-isopropyl-6-(2-methoxypyridin-4-yl)phenyl)acetyl)-hydrazinecarbothioamide (2.6 g, 5.30 mmol, 1 eq, TFA salt) in H₂O (70 mL) was added NaOH (2.60 g, 65.00 mmol, 12.26 eq). The mixture was stirred at 100° C. for 2 hours. The reaction mixture was diluted with H₂O (20 mL), adjusted to pH=6-7 with 1 N HCl aqueous solution and extracted with EtOAc (3×50 mL). The organic phases were dried over anhydrous Na₂SO₄, filtered and concentrated in vacuum. The residue was purified by prep-HPLC (column: Phenomenex luna C18, 250 mm*50 mm*10 μm; mobile phase: [A: water (0.1% TFA v/v), B: MeCN]; B %: 25%-55%, 25 min) to give the title compound (1.4 g, 73.7% yield) as an off-white solid.

¹H NMR (400 MHz, DMSO-d₆) δ 13.23 (br s, 1H), 13.07 (br s, 1H), 8.19 (d, 1H), 7.25 (dd, 1H), 6.94-6.86 (m, 2H), 6.71 (s, 1H), 3.85 (s, 3H), 3.74 (s, 2H), 2.96-2.93 (m, 1H), 1.11 (d, 6H).

LCMS: m/z 359.3 (M+H)⁺ (ES⁺).

### Intermediate R16:5-(4-fluoro-2-isopropyl-6-(2-methoxypyridin-4-yl)benzyl)-4H-1,2,4-triazole-3-sulfonylchloride

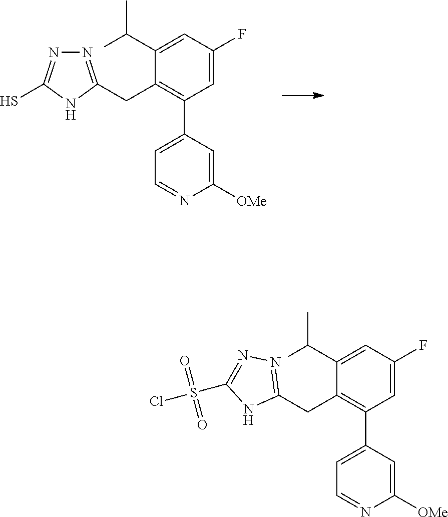 [SMILES: COc1cc(-c2cc(F)cc(C(C)C)c2Cc2nnc(S)[nH]2)ccn1 ; COc1cc(-c2cc(F)cc(C(C)C)c2Cc2nnc(S(=O)(=O)Cl)[nH]2)ccn1]

To a soluiton of 5-(4-fluoro-2-isopropyl-6-(2-methoxypyridin-4-yl)benzyl)-4H-1,2,4-triazole-3-thiol (Intermediate R15) (200 mg, 557.99 μmol, 1 eq) in H₂O (1 mL) and AcOH (4 mL) was added NCS (223 mg, 1.67 mmol, 3 eq). The mixture was stirred at 25° C. for 12 hours. The reaction solution was diluted with DCM (20 mL) and washed with H₂O (20 mL). The organic phases were dried over anhydrous Na₂SO₄, filtered and concentrated in vacuum to give the title compound (120 mg, crude) as yellow oil.

### Intermediate R17:5-((1,2,3,5,6,7-hexahydro-s-indacen-4-yl)methyl)-4H-1,2,4-triazole-3-sulfonylchloride

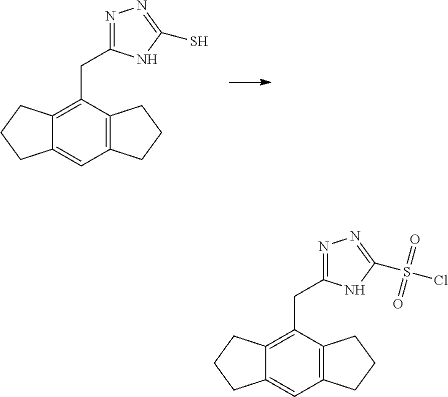 [SMILES: Sc1nnc(Cc2c3c(cc4c2CCC4)CCC3)[nH]1 ; O=S(=O)(Cl)c1nnc(Cc2c3c(cc4c2CCC4)CCC3)[nH]1]

To a solution of 5-((1,2,3,5,6,7-hexahydro-s-indacen-4-yl)methyl)-4H-1,2,4-triazole-3-thiol (Intermediate R3) (100 mg, 368.49 μmol, 1 eq) in AcOH (5 mL) and H₂O (1 mL) was added NCS (148 mg, 1.11 mmol, 3 eq). The mixture was stirred at 25° C. for 12 hours. The reaction mixture was diluted with DCM (10 mL) and washed with H₂O (3×5 mL) and brine (3×5 mL). The organic layer was dried over anhydrous Na₂SO₄, filtered and concentrated in vacuum to give the title compound (100 mg, crude) as a yellow oil.

### Intermediate R8: 5-((5-(2-methoxypyridin-4-yl)-2,3-dihydro-1H-inden-4-yl)methyl)-4H-1,2,4-triazole-3-sulfonyl chloride

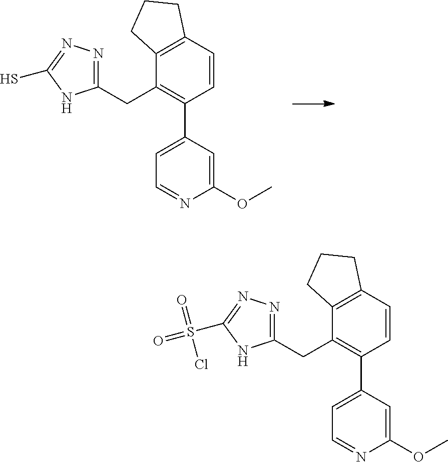 [SMILES: COc1cc(-c2ccc3c(c2Cc2nnc(S)[nH]2)CCC3)ccn1 ; COc1cc(-c2ccc3c(c2Cc2nnc(S(=O)(=O)Cl)[nH]2)CCC3)ccn1]

NCS (189 mg, 1.42 mmol, 3 eq) was added into a solution of 5-((5-(2-methoxypyridin-4-yl)-2,3-dihydro-1H-inden-4-yl)methyl)-4H-1,2,4-triazole-3-thiol (Intermediate R⁵) (160 mg, 472.78 μmol, 1 eq) in AcOH (5 mL) and H₂O (1 mL) at 20° C. Then the mixture was stirred for 12 hours at 20° C. The reaction mixture was poured into water (20 mL) and extracted with DCM (3×20 mL). The combined organic phases were washed with brine (3×30 mL), dried over anhydrous Na₂SO₄, filtered and concentrated in vacuum to give the title compound (190 mg, crude) as a yellow oil.

### Intermediate R19: 5-(4-fluoro-2,6-diisopropylbenzyl)-4H-1,2,4-triazole-3-sulfonyl chloride

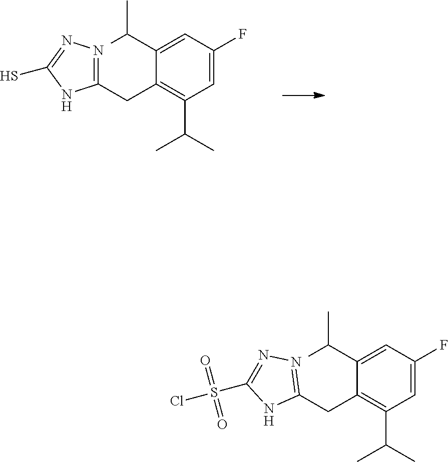 [SMILES: CC(C)c1cc(F)cc(C(C)C)c1Cc1nnc(S)[nH]1 ; CC(C)c1cc(F)cc(C(C)C)c1Cc1nnc(S(=O)(=O)Cl)[nH]1]

To a mixture of 5-(4-fluoro-2,6-diisopropylbenzyl)-4H-1,2,4-triazole-3-thiol (Intermediate R2) (200 mg, 681.66 μmol, 1 eq) in AcOH (8 mL) and H₂O (2 mL) was added NCS (91 mg, 30.34 mmol, 44.51 eq) in one portion. Then the reaction mixture was stirred at 20° C. for 12 hours. The mixture was quenched with water (20 mL) and extracted with DCM (3×20 mL). The combined organic phases were washed with brine (3×20 mL), dried over anhydrous Na₂SO₄, filtered and concentrated in vacuum to give the title compound (0.25 g, crude) as a yellow oil.

### Intermediate A1: 3-bromo-5-(phenylsulfonyl)-1-((2-(trimethylsilyl)ethoxy)methyl)-1H-1,2,4-triazole

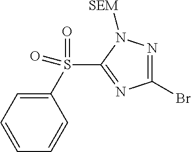 [SMILES: C[Si](C)(C)CCOCn1nc(Br)nc1S(=O)(=O)c1ccccc1]

### Step A: 3-bromo-5-(phenylthio)-1-((2-(trimethylsilyl)ethoxy)methyl)-1H-1,2,4-triazole

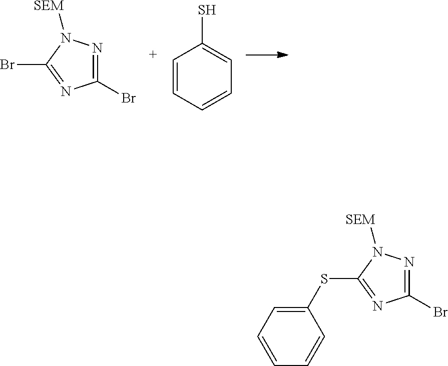 [SMILES: C[Si](C)(C)CCOCn1nc(Br)nc1Br ; Sc1ccccc1 ; C[Si](C)(C)CCOCn1nc(Br)nc1Sc1ccccc1]

Benzenethiol (0.32 mL, 3.08 mmol), DBU (0.84 mL, 5.60 mmol) and DMF (10 mL) were stirred for 10 min before adding 3,5-dibromo-1-((2-(trimethylsilyl)ethoxy)-methyl)-1H-1,2,4-triazole (1.00 g, 2.80 mmol). The reaction was stirred at RT for 5 h, diluted with EtOAc (50 mL) and washed with aq 1 M HCl (50 mL), water (50 mL) and aq 2 M NaOH (50 mL). The organic extract was dried (phase separator) and concentrated in vacuo. The product was purified by chromatography on silica gel (24 g column, 0-50% EtOAc/isohexane) to afford the title compound (1.1 g, 92% yield) as a clear colourless oil.

¹H NMR (DMSO-d₆) δ 7.55-7.50 (m, 2H), 7.49-7.42 (m, 3H), 5.54 (s, 2H), 3.61-3.55 (m. 2H), 0.86-0.81 (m. 2H), −0.05 (s, 9H).

### Step B: 3-bromo-5-(phenylsulfonyl)-1-((2-(trimethylsilyl)ethoxy)methyl)-1H-1,2,4-triazole

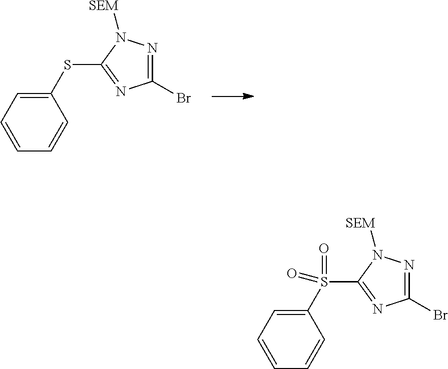 [SMILES: C[Si](C)(C)CCOCn1nc(Br)nc1Sc1ccccc1 ; C[Si](C)(C)CCOCn1nc(Br)nc1S(=O)(=O)c1ccccc1]

To an ice-cooled solution of 3-bromo-5-(phenylthio)-1-((2-(trimethylsilyl)ethoxy)-methyl)-1H-1,2,4-triazole (1.0 g, 2.59 mmol) in DCM (15 mL) was added m-CPBA (447 mg, 2.59 mmol) and the reaction was stirred for 15 min at 0° C. and allowed to warm to RT. Two further portions of m-CPBA (0.67 g, 3.88 mmol) were added at 0° C. after 1 h and 5 h. The reaction mixture was then stirred at RT overnight and quenched with a sodium sulfite solution. The aqueous layer was extracted with DCM (3×30 mL) and the organics washed with sat aq NaHCO₃, dried (phase separator) and concentrated in vacuo to afford the title compound (1.09 g, 96% yield) as a clear colourless oil.

LCMS: no ionization

¹H NMR (DMSO-d₆) δ 8.11-8.07 (m, 2H), 7.91-7.84 (m, 1H), 7.78-7.72 (m, 2H), 5.85 (s, 2H), 3.62-3.54 (m, 2H), 0.82-0.74 (m, 2H), −0.06 (s, 9H).

### Intermediate A2: 1-(4-((3-bromo-1-(4-methoxybenzyl)-1H-1,2,4-triazol-5-yl)sulfonyl)phenyl)-N,N-dimethylmethanamine

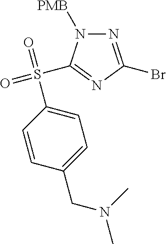

### Step A: lithium 4-((dimethylamino)methyl)benzenesulfinate

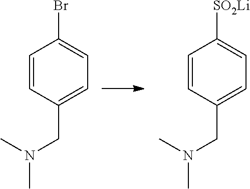

ⁿBuLi (2.5 M in hexanes, 2.15 mL, 5.37 mmol) was added dropwise to a solution of 1-(4-bromophenyl)-N,N-dimethylmethanamine (1.0 g, 4.67 mmol) in THF (10 mL) at −78° C. and the reaction was stirred for 15 min. SO₂ was bubbled through the solution for 5 min, then the reaction was warmed to RT. After 2 h, the reaction was concentrated to almost dryness then triturated with MTBE (40 mL) and the solid was filtered to afford the title compound (0.8 g, 71% yield) as a yellow solid.

¹H NMR (DMSO-d₆) δ 7.45 (d, J=7.7 Hz, 2H), 7.30 (d, J=7.6 Hz, 2H), 3.62 (s, 2H), 2.20 (s, 6H).

### Step B: 1-(4-((3-bromo-1-(4-methoxybenzyl)-1H-1,2,4-triazol-5-yl)sulfonyl)phenyl)-N,N-dimethylmethanamine

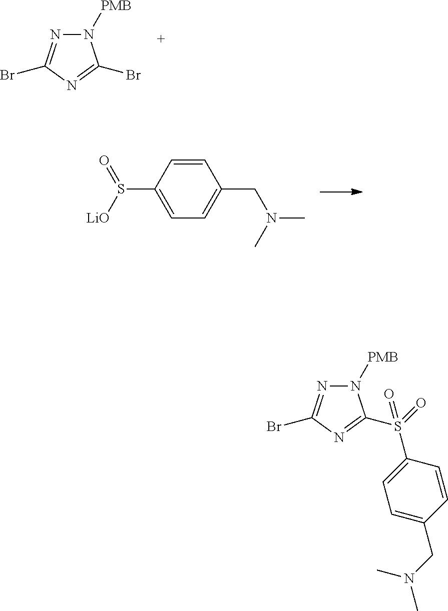

A mixture of 3,5-dibromo-1-(4-methoxybenzyl)-1H-1,2,4-triazole (CAS 372162-76-0) (579 mg, 1.67 mmol) and lithium 4-((dimethylamino)methyl)benzenesulfinate (750 mg, 2.74 mmol) in DMF (3.5 mL) was heated at 100° C. for 5 h. The mixture was diluted with EtOAc (100 mL) and washed with brine/water (3:1, 70 mL). The aqueous phase was extracted with MTBE (70 mL) and the combined organics dried (MgSO₄), filtered and concentrated in vacuo. The crude product was loaded onto a column of SCX (18.4 g) in DCM. The column was washed with MeOH/DCM (1:1, 60 mL) and the product was eluted with 0.7 M ammonia in MeOH (100 mL). The solvent was evaporated in vacuo to afford the title compound (275 mg, 34% yield) as a cloudy oil.

LCMS m/z 465.2, 467.2 (M+H)⁺ (ES⁺).

¹H NMR (DMSO-d₆) δ 7.94 (d, J=8.4 Hz, 2H), 7.59 (d, J=8.2 Hz, 2H), 7.22 (d, J=8.7 Hz, 2H), 6.92 (d, J=8.7 Hz, 2H), 5.70 (s, 2H), 3.74 (s, 3H), 3.51 (s, 2H), 2.16 (s, 6H).

### Intermediate A3: 1-(3-((3-bromo-1-(4-methoxybenzyl)-1H-1,2,4-triazol-5-yl)sulfonyl)phenyl)-N,N-dimethylmethanamine

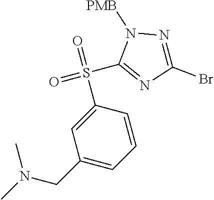

### Step A: lithium 3-((dimethylamino)methyl)benzenesulfinate

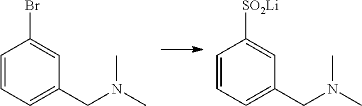

Prepared according to the general procedure of lithium 4-((dimethylamino)methyl)-benzenesulfinate (Intermediate A2, Step A) from 1-(3-bromophenyl)-N,N-dimethylmethanamine to afford the title compound (2.56 g, 100% yield) as a yellow solid.

¹H NMR (DMSO-d₆) δ 7.48 (s, 1H), 7.42 (d, J=7.5 Hz, 1H), 7.30 (t, J=7.4 Hz, 1H), 7.22 (d, J=7.5 Hz, 1H), 3.65-3.55 (m, 2H), 2.20 (s, 6H).

### Step B: 1-(3-((3-bromo-1-(4-methoxybenzyl)-1H-1,2,4-triazol-5-yl)sulfonyl)phenyl)-N,N-dimethylmethanamine

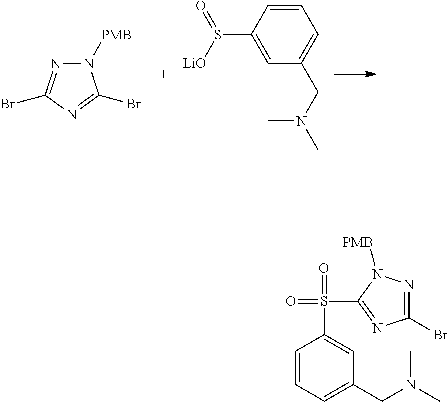

Prepared according to the general procedure of 1-(4-((3-bromo-1-(4-methoxybenzyl)-1H-1,2,4-triazol-5-yl)sulfonyl)phenyl)-N,N-dimethylmethanamine (Intermediate A2, Step B) from 3,5-dibromo-1-(4-methoxybenzyl)-1H-1,2,4-triazole and lithium 3-((dimethylamino)methyl)benzenesulfinate to afford the title compound (150 mg, 20% yield) as a colourless oil.

LCMS m/z 465.0, 467.0 (M+H)⁺ (ES⁺).

¹H NMR (DMSO-d₆) δ 7.87 (d, J=8.1 Hz, 1H), 7.79 (s, 1H), 7.74 (d, J=7.8 Hz, 1H), 7.64 (t, J=7.8 Hz, 1H), 7.22 (d, J=8.7 Hz, 2H), 6.93 (d, J=8.7 Hz, 2H), 5.71 (s, 2H), 3.75 (s, 3H), 3.45 (s, 2H), 2.13 (s, 6H).

### Intermediate A4:5-bromo-3-(phenylsulfonyl)-1-((2-(trimethylsilyl)ethoxy)methyl)-1H-1,2,4-triazole

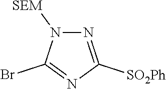

### Step A4: 3-bromo-1-(4-methoxybenzyl)-5-(phenylsulfonyl)-1H-1,2,4-triazole

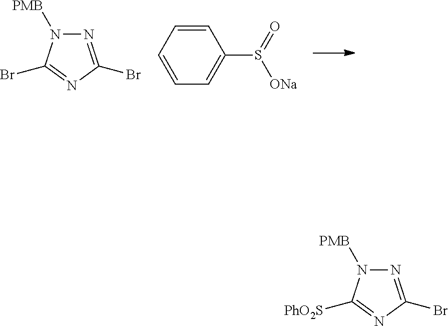

A mixture of 3,5-dibromo-1-(4-methoxybenzyl)-1H-1,2,4-triazole (2.00 g, 5.76 mmol) and sodium benzenesulfinate (1.42 g, 8.65 mmol) in DMF (30 mL) was heated at 100° C. for 4 h. The reaction mixture was diluted with EtOAc (100 mL) and washed with water/brine (1:1, 80 mL). The organic phase was separated, dried (MgSO₄), filtered and concentrated in vacuo. The crude product was purified by chromatography on silica gel (220 g column, 0-50% EtOAc/isohexane) to afford the title compound (1.17 g, 44% yield) as a white solid.

LCSM m/z 407.9, 409.9 (M+H)⁺ (ES⁺).

¹H NMR (DMSO-d₆) δ 8.01-7.96 (m, 2H), 7.86 (t, J=7.5 Hz, 1H), 7.70 (t, J=7.9 Hz, 2H), 7.24 (d, J=8.6 Hz, 2H), 6.93 (d, J=8.6 Hz, 2H), 5.71 (s, 2H), 3.75 (s, 3H).

### Step B: 3-bromo-5-(phenylsulfonyl)-1H-1,2,4-triazole

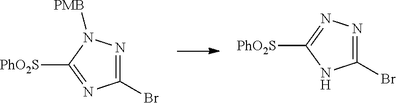

3-Bromo-1-(4-methoxybenzyl)-5-(phenylsulfonyl)-1H-1,2,4-triazole (1.18 g, 2.89 mmol) was dissolved in TFA (5 mL, 64.9 mmol) and heated to 100° C. overnight. The reaction mixture was concentrated in vacuo. The product was purified by chromatography on silica gel (12 g column, 0-100% EtOAc/isohexane) to afford the title compound (983 mg, 99% yield) as a thick green oil.

LCMS m/z 289.8 (M+H)⁺ (ES⁺).

¹H NMR (DMSO-d₆) δ 8.01-7.96 (m, 2H), 7.84-7.78 (m, 1H), 7.70 (t, J=7.8 Hz, 2H). One exchangeable proton not observed.

### Step C: 5-bromo-3-(phenylsulfonyl)-1-((2-(trimethylsilyl)ethoxy)methyl)-1H-1,2,4-triazole

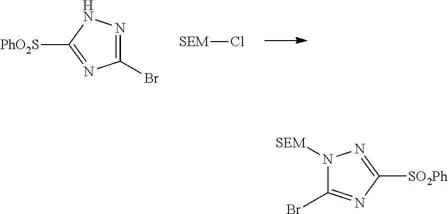

SEM-Cl (0.684 mL, 3.85 mmol) was added to a solution of 3-bromo-5-(phenylsulfonyl)-1H-1,2,4-triazole (0.983 g, 3.41 mmol), K₂CO₃ (990 mg, 7.16 mmol), and MeCN (10 mL) at 0° C. The reaction was warmed to RT and stirred overnight then filtered and concentrated in vacuo. The product was purified by chromatography on silica gel (24 g column, 0-20% EtOAc/isohexane) to afford the title compound (462 mg, 29% yield) as a clear yellow oil.

¹H NMR (DMSO-d₆) 7.99 (d, J=7.2 Hz, 2H), 7.82 (t, J=7.2 Hz, 1H), 7.74-7.67 (m, 2H), 5.57 (s, 2H), 3.59 (t, J=7.9 Hz, 2H), 0.81 (t, J=8.0 Hz, 2H), −0.10 (s, 9H). Regioselectivity was confirmed by HMBC-NMR.

Note: 3-bromo-5-(phenylsulfonyl)-1-((2-(trimethylsilyl)ethoxy)methyl)-1H-1,2,4-triazole (Intermediate A1) (440 mg, 28% yield) was also isolated from this reaction.

### Intermediate A5: 2-((3-bromo-1-((2-(trimethylsilyl)ethoxy)methyl)-1H-1,2,4-triazol-5-yl)sulfonyl)-N,N-diethylethanamine

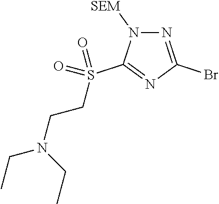 [SMILES: CCN(CC)CCS(=O)(=O)c1nc(Br)nn1COCC[Si](C)(C)C]

### Step A: 2-((3-bromo-1-((2-(trimethylsilyl)ethoxy)methyl)-1H-1,2,4-triazol-5-yl)thio)ethanol

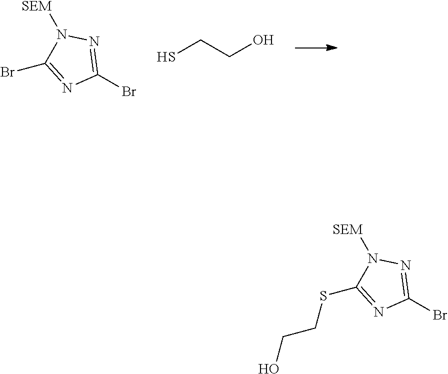

To a stirred solution of 2-mercaptoethanol (0.393 mL, 5.60 mmol) in DMF (15 mL) at 0° C. was added NaH (60% in mineral oil, 246 mg, 6.16 mmol) portionwise. The mixture was stirred at 0° C. for 10 min, then a solution of 3,5-dibromo-1-((2-(trimethylsilyl)-ethoxy)methyl)-1H-1,2,4-triazole (2.00 g, 5.60 mmol) in DMF (5 mL) was added dropwise. The reaction was stirred at RT for 3 h and then poured into water (25 mL). The product was extracted with EtOAc (3×25 mL) and the combined organics were washed with brine (50 mL), dried (MgSO₄) and concentrated in vacuo. The crude was purified by chromatography on silica gel (80 g column, 0-40% EtOAc/isohexane) to afford the title compound (1.40 g, 61% yield) as a pale yellow oil.

LCMS m/z 354.1 and 356.1 (M+H)⁺ (ES⁺).

¹H NMR (DMSO-d₆) δ 5.37 (s, 2H), 5.07 (t, J=5.5 Hz, 1H), 3.66-3.62 (m, 2H), 3.61-3.55 (m 2H), 0.29 (t, J=6.4 Hz, 2H), 0.89-0.81 (m. 2H), −0.03 (s, 9H).

### Step B: 2-((3-bromo-1-((2-(trimethylsilyl)ethoxy)methyl)-1H-1,2,4-triazol-5-yl)sulfonyl)ethanol

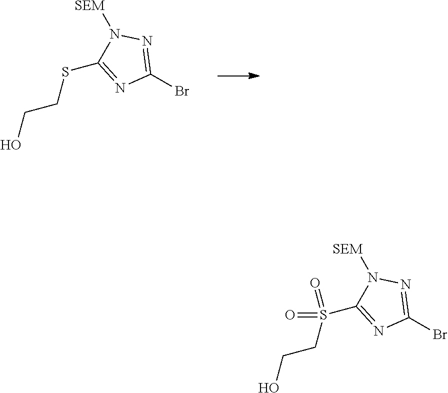 [SMILES: C[Si](C)(C)CCOCn1nc(Br)nc1SCCO ; C[Si](C)(C)CCOCn1nc(Br)nc1S(=O)(=O)CCO]

To a stirred solution of 2-((3-bromo-1-((2-(trimethylsilyl)ethoxy)methyl)-1H-1,2,4-triazol-5-yl)thio)ethanol (1.40 g, 3.95 mmol) in DCM (20 mL) at 0° C. was added m-CPBA (2.77 g, 12.4 mmol) in 5 portions over 2 days. The reaction was stirred at RT between the additions and monitored by LCMS. The reaction was quenched with aq sodium sulfite solution. The product was extracted with DCM (3×30 mL) and the combined organics washed with sat aq NaHCO₃, dried (MgSO₄) and concentrated in vacuo to afford the title compound (1.21 g, 77% yield) as a colourless oil.

¹H NMR (DMSO-d₆) δ 5.73 (s, 2H), 5.03 (t, J=4.9 Hz, 1H), 3.85-3.78 (m, 4H), 3.67-3.64 (m, 2H), 0.88-0.85 (m, 2H), −0.04 (s, 9H).

### Step C: 2-((3-bromo-1-((2-(trimethylsilyl)ethoxy)methyl)-1H-1,2,4-triazol-5-yl)sulfonyl)-N,N-diethylethanamine

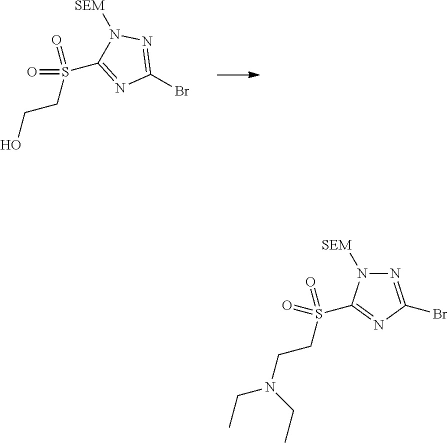 [SMILES: C[Si](C)(C)CCOCn1nc(Br)nc1S(=O)(=O)CCO ; CCN(CC)CCS(=O)(=O)c1nc(Br)nn1COCC[Si](C)(C)C]

To a stirred solution of 2-((3-bromo-1-((2-(trimethylsilyl)ethoxy)methyl)-1H-1,2,4-triazol-5-yl)sulfonyl)ethanol (400 mg, 1.04 mmol) and DIPEA (253 μL, 1.45 mmol) in DCM (4 mL) at 0° C. was added MsCl (97 μL, 1.24 mmol). The reaction was stirred at 0° C. for 30 min and then at RT for 1 h. The reaction mixture was partitioned between DCM (10 mL) and sat aq NaHCO₃ (10 mL). The organics were dried (phase separator) and concentrated in vacuo. The residue was dissolved in THF (4 mL) and Et₂NH (541 μL, 5.18 mmol) was added. The reaction was stirred at RT for 2 h and concentrated in vacuo. The residue was dissolved in MeOH and loaded onto a column of SCX (5 g). The column was washed with MeOH (50 mL) and the product was eluted with 0.7 M ammonia in MeOH (so mL. The solvent was evaporated in vacuo and the product was purified by chromatography on silica gel (24 g column, 0-30% EtOAc/isohexane) to afford the title compound (243 mg, 53% yield) as a clear colourless oil.

LCMS m/z 441.0 and 443.3 (M+H)⁺ (ES⁺).

¹H NMR (DMSO-d₆) δ 5.75 (s, 2H), 3.82 (t, J=6.3 Hz, 2H), 3.71-3.63 (m, 2H), 2.84 (t, J=6.4 Hz, 2H), 2.32 (q, J=7.1 Hz, 4H), 0.91-0.84 (m, 2H), 0.77 (t, J=7.1 Hz, 6H), −0.03 (s, 9H).

### Intermediate A6:3-((3-bromo-1-((2-(trimethylsilyl)ethoxy)methyl)-1H-1,2,4-triazol-5-yl)sulfonyl)propan-1-ol

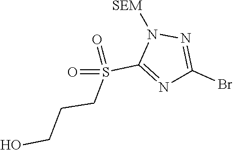 [SMILES: C[Si](C)(C)CCOCn1nc(Br)nc1S(=O)(=O)CCCO]

### Step A: 3-((3-bromo-1-((2-(trimethylsilyl)ethoxy)methyl)-1H-1,2,4-triazol-5-yl)thio)propan-1-ol

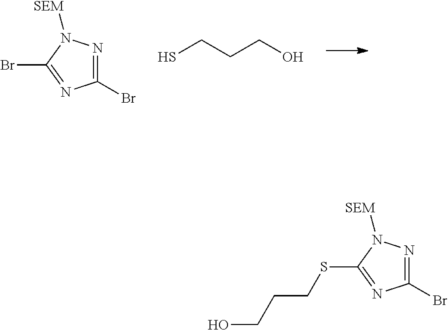 [SMILES: C[Si](C)(C)CCOCn1nc(Br)nc1Br ; OCCCS ; C[Si](C)(C)CCOCn1nc(Br)nc1SCCCO]

Prepared according to the general procedure of 2-((3-bromo-1-((2-(trimethylsilyl)-ethoxy)methyl)-1H-1,2,4-triazol-5-yl)thio)ethanol (Intermediate A5, Step A) from 3-mercaptopropan-1-ol and 3,5-dibromo-1-((2-(trimethylsilyl)ethoxy)methyl)-1H-1,2,4-triazole to afford the title compound (2.54 g, 81% yield) as a colourless liquid.

¹H NMR (CDCl₃) δ 5.34 (s, 2H), 3.74 (t, J=5.5 Hz, 2H), 3.64-3.61 (m, 2H), 3.40 (t, J=6.5 Hz, 2H), 2.89 (br s, 1H), 2.00-1.95 (m, 2H), 0.94-0.91 (m, 2H), −0.01 (s, 9H).

### Step B: 3-((3-bromo-1-((2-(trimethylsilyl)ethoxy)methyl)-1H-1,2,4-triazol-5-yl)sulfonyl)propan-1-ol

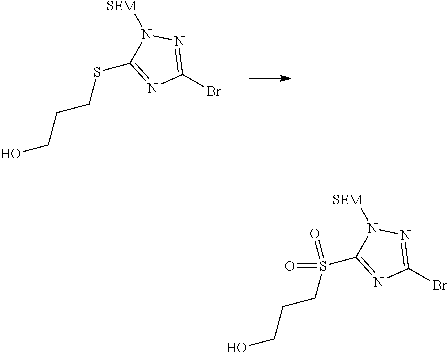 [SMILES: C[Si](C)(C)CCOCn1nc(Br)nc1SCCCO ; C[Si](C)(C)CCOCn1nc(Br)nc1S(=O)(=O)CCCO]

Prepared according to the general procedure of 2-((3-bromo-1-((2-(trimethylsilyl)-ethoxy)methyl)-1H-1,2,4-triazol-5-yl)sulfonyl)ethanol (Intermediate A5, Step B) from 3-((3-bromo-1-((2-(trimethylsilyl)ethoxy)methyl)-1H-1,2,4-triazol-5-yl)thio)-propan-1-ol to afford the title compound (2.51 g, 87% yield) as a colourless oil.

¹H NMR (DMSO-d₆) δ 5.75 (s, 2H), 4.72 (t, J=5.3 Hz, 1H), 3.70-3.63 (m, 4H), 3.47 (app q, J=5.9 Hz, 2H), 1.86-1.80 (m, 2H), 0.88-0.85 (m, 2H), −0.04 (s, 9H).

### Intermediate A7: 3-((3-bromo-1-((2-(trimethylsilyl)ethoxy)methyl)-1H-1,2,4-triazol-5-yl)sulfonyl)-N,N-diethylpropan-1-amine

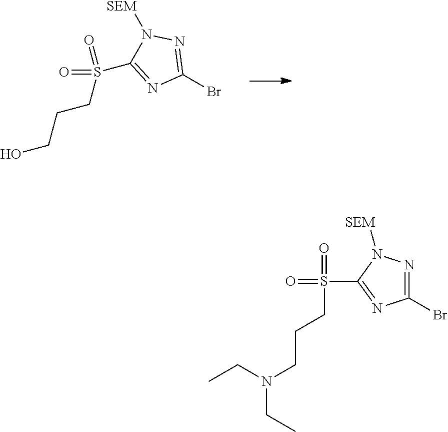 [SMILES: C[Si](C)(C)CCOCn1nc(Br)nc1S(=O)(=O)CCCO ; CCN(CC)CCCS(=O)(=O)c1nc(Br)nn1COCC[Si](C)(C)C]

To a stirred solution of 3-((3-bromo-1-((2-(trimethylsilyl)ethoxy)methyl)-1H-1,2,4-triazol-5-yl)sulfonyl)propan-1-ol (Intermediate A6) (1.0 g, 2.50 mmol) and DIPEA (0.611 mL, 3.50 mmol) in THF (10 mL) at 0° C. was added MsCl (0.204 mL, 2.62 mmol) dropwise and the reaction stirred for 1 h. KI (41 mg, 0.250 mmol) and diethylamine (2.61 mL, 25.0 mmol) were added. The reaction was stirred at 40° C. for 2 h then heated at 50° C. for 2 h and then at 60° C. for 16 h before being concentrated in vacuo. The residue was taken up in EtOAc (40 mL), washed with water (50 mL), dried (phase separator) and concentrated in vacuo. The product was purified by column chromatography on silica gel (24 g column, 0-10% MeOH/DCM) to afford the title compound (609 mg, 54% yield) as a yellow/orange oil.

¹H NMR (CDCl₃) δ 5.79 (s, 2H), 3.72-3.69 (m, 2H), 3.63-3.60 (m, 2H), 2.62-2.56 (m, 6H), 2.04 (br s, 2H), 1.04 (t, J=7.3 Hz, 6H), 0.96-0.92 (m, 2H), 0.00 (s, 9H).

### Intermediate A8:3-bromo-N-(2-(diethylamino)ethyl)-N-methyl-1-((2-(trimethylsilyl)ethoxy)methyl)-1H-1,2,4-triazole-5-sulfonamide

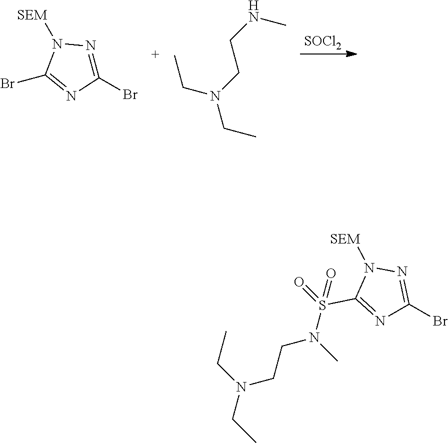

ⁿBuLi (2.5 M in hexanes, 0.715 mL, 1.79 mmol) was added dropwise to a solution of 3,5-dibromo-1-((2-(trimethylsilyl)ethoxy)methyl)-1H-1,2,4-triazole (0.58 g, 1.62 mmol) in THF (12 mL) at −78° C. and the reaction was stirred for 15 min. SO₂Cl₂ (0.145 mL, 1.79 mmol) was added dropwise and the solution was stirred at −78° C. for 1 h. N¹,N¹-Diethyl-N²-methylethane-1,2-diamine (0.289 mL, 1.79 mmol) was added and the reaction was allowed to warm to RT over 1.5 h. Two portions of N¹,N¹-diethyl-N²-methylethane-1,2-diamine (0.289 mL, 1.79 mmol) were added with a 30 min interval and the reaction was stirred for 30 min, diluted with EtOAc (100 mL) and washed with water (100 mL). The organic phase was dried (MgSO₄) and concentrated in vacuo. The product was purified by chromatography on silica gel (40 g column, 0-5% MeOH/DCM) followed by SCX column, eluting with DCM/MeOH (40 mL) then 0.7 N NH₃ in MeOH (40 mL). The ammoniacal solution was concentrated in vacuo to afford the title compound (170 mg, 22% yield) as a yellow oil.

LCMS m/z 470.1, 472.1 (M+H)⁺ (ES⁺).

¹H NMR (DMSO-d₆) δ 5.70 (s, 2H), 3.70-3.60 (m, 2H), 3.00 (s, 3H), 2.58 (br s, 2H), 2.46 (br s, 4H), 0.92 (t, J=7.1 Hz, 6H), 0.88-0.83 (m, 2H), −0.04 (s, 9H). One CH₂ obscured by solvent.

### Intermediate A9: 3-bromo-N-(2-(diethylamino)ethyl)-1-((2-(trimethylsilyl)ethoxy)methyl)-1H-1,2,4-triazole-5-sulfonamide

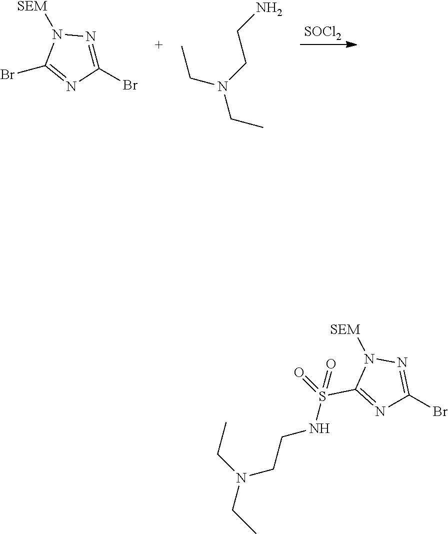

ⁿBuLi (2.5 M in hexanes, 1.23 mL, 3.08 mmol) was added dropwise to a solution of 3,5-dibromo-1-((2-(trimethylsilyl)ethoxy)methyl)-1H-1,2,4-triazole (1.00 g, 2.80 mmol) in THF (20 mL) at −78° C. and the reaction was stirred for 15 min. SO₂Cl₂ (0.250 mL, 3.08 mmol) was added dropwise at −78° C. and the solution was stirred at this temperature for 1 h. A solution of N¹,N¹-diethylethane-1,2-diamine (0.787 mL, 5.60 mmol) in Et₃N (1.17 mL, 8.40 mmol) was added and warmed to RT over 1.5 h. The reaction was diluted with EtOAc (100 mL) and washed with water (75 mL). The aqueous phase was extracted with EtOAc (100 mL) and the combined organics were dried (MgSO₄) and concentrated in vacuo. The crude was passed through SCX (15 g), eluting with DCM/MeOH (1:1, 50 mL), then with 0.7 N NH₃ in MeOH (50 mL). The ammoniacal phase was concentrated in vacuo to afford the title compound (0.404 g, 28% yield) as a yellow oil.

LCMS m/z 456.0, 458.0 (M+H)⁺ (ES⁺).

### Intermediate A10: (S)-2-(1-((3-bromo-1-((2-(trimethylsilyl)ethoxy)methyl)-1H-1,2,4-triazol-5-yl)sulfonyl)pyrrolidin-3-yl)propan-2-ol

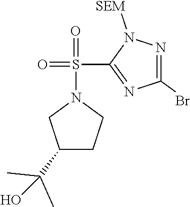 [SMILES: CC(C)(O)[C@H]1CCN(S(=O)(=O)c2nc(Br)nn2COCC[Si](C)(C)C)C1]

### Step A: (S)-1-tert-butyl 3-methyl pyrrolidine-1,3-dicarboxylate

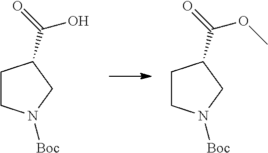 [SMILES: CC(C)(C)OC(=O)N1CC[C@H](C(=O)O)C1 ; COC(=O)[C@H]1CCN(C(=O)OC(C)(C)C)C1]

MeI (7.81 mL, 125 mmol) was added to a suspension of (S)-1-(tert-butoxycarbonyl)-pyrrolidine-3-carboxylic acid (9.0 g, 41.8 mmol) and K₂CO₃ (17.3 g, 125 mmol) in DMF (100 mL). The reaction was stirred at RT for 20 h then partitioned between EtOAc (100 mL) and brine (100 mL) and the phases separated. The aqueous phase was extracted with EtOAc (100 mL) and the organic phases were combined, dried (MgSO₄) and concentrated in vacuo to afford the title compound (9.56 g, 98%) as an orange oil.

¹H NMR (CDCl₃) δ 3.70 (s, 3H), 3.64-3.55 (m, 1H), 3.54-3.42 (m, 2H), 3.40-3.27 (m, 1H), 3.03 (p, J=7.6 Hz, 1H), 2.11 (q, J=7.2 Hz, 2H), 1.44 (s, 9H).

### Step B: (S)-tert-butyl 3-(2-hydroxypropan-2-yl)pyrrolidine-1-carboxylate

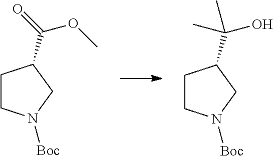 [SMILES: COC(=O)[C@H]1CCN(C(=O)OC(C)(C)C)C1 ; CC(C)(C)OC(=O)N1CC[C@H](C(C)(C)O)C1]

MeMgBr (3 M in Et₂O) (41.0 mL, 123 mmol) was added dropwise to a solution of (S)-1-tert-butyl 3-methyl pyrrolidine-1,3-dicarboxylate (9.4 g, 41.0 mmol) in THF (100 mL) at 0° C. and the reaction was stirred at RT for 1 h. The reaction was quenched with sat. aq. NH₄Cl (100 mL) and extracted with EtOAc (150 mL). The organic phase was separated, dried (MgSO₄) and concentrated in vacuo to afford the title compound (8.6 g, 87%) as a pale yellow solid.

¹H NMR (DMSO-d₆) δ 4.31-4.30 (m, 1H), 3.40-3.33 (m, 1H), 3.29-3.23 (m, 1H), 3.16-3.00 (m, 2H), 2.17-2.04 (m, 1H), 1.80-1.61 (m, 2H), 1.39 (d, J=1.9 Hz, 9H), 1.07 (s, 6H).

### Step C: (S)-2-(pyrrolidin-3-yl)propan-2-ol

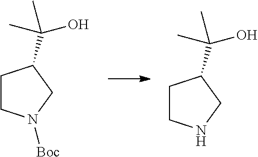

A solution of (S)-tert-butyl 3-(2-hydroxypropan-2-yl)pyrrolidine-1-carboxylate (3 g, 13.08 mmol) in DCM (20 mL) and TFA (15 mL) was stirred at RT for 3 h. The volatiles were removed in vacuo and the crude product was loaded onto a column of SCX (15 g) in MeOH/DCM. The column was washed with MeOH and the product was eluted with 7 M ammonia in MeOH. The ammoniacal solution was concentrated in vacuo to afford the title compound (1 g, 58%) as a thick orange oil.

¹H NMR (DMSO-d₆) δ 2.77-2.65 (m, 3H), 2.60 (dd, J=10.7, 8.0 Hz, 1H), 1.95 (p, J=8.4 Hz, 1H), 1.65-1.57 (m, 1H), 1.54-1.45 (m, 1H), 1.05 (s, 6H). Two exchangable protons not observed.

### Step D: (S)-2-(1-((3-bromo-1-((2-(trimethylsilyl)ethoxy)methyl)-1H-1,2,4-triazol-5-yl)sulfonyl)pyrrolidin-3-yl)propan-2-ol

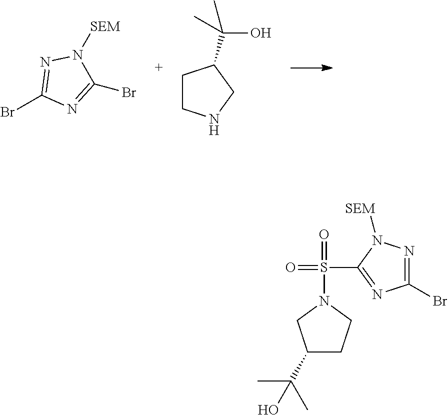

Prepared according to the general procedure of 3-bromo-N-(2-(diethylamino)ethyl)-1-((2-(trimethylsilyl)ethoxy)methyl)-1H-1,2,4-triazole-5-sulfonamide (Intermediate A9) from 3,5-dibromo-1-((2-(trimethylsilyl)ethoxy)methyl)-1H-1,2,4-triazole and (S)-2-(pyrrolidin-3-yl)propan-2-ol to afford the title compound (1.20 g, 30%) as a thick orange oil.

¹H NMR (CDCl₃) δ 5.73 (s, 2H), 3.78-3.62 (m, 4H), 3.55-3.44 (m, 2H), 2.40 (p, J=8.8 Hz, 1H), 2.04-1.95 (m, 2H), 1.24 (d, J=5.0 Hz, 6H), 1.01-0.90 (m, 2H), −0.00 (s, 9H). One exchangable proton not observed.

### Intermediate A: 1-((3-bromo-1-((2-(trimethylsilyl)ethoxy)methyl)-1H-1,2,4-triazol-5-yl)sulfonyl)-N,N-dimethylpiperidin-4-amine

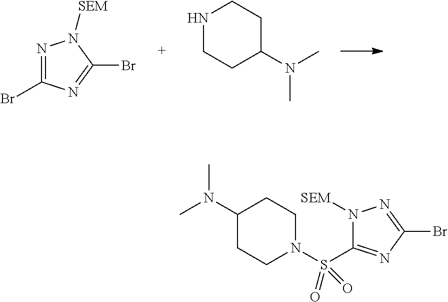

Prepared according to the general procedure of 3-bromo-N-(2-(diethylamino)ethyl)-1-((2-(trimethylsilyl)ethoxy)methyl)-1H-1,2,4-triazole-5-sulfonamide (Intermediate A9) from 3,5-dibromo-1-((2-(trimethylsilyl)ethoxy)methyl)-1H-1,2,4-triazole and N,N-dimethylpiperidin-4-amine to afford the title compound (2.53 g, 30%).

LCMS m/z 468.2/470.3 (M+H)⁺ (ES⁺).

¹H NMR (DMSO-d₆) δ 5.71 (s, 2H), 3.81-3.72 (m, 2H), 3.65 (t, J=8.9 Hz, 2H), 3.03-2.90 (t, J=8.1 Hz, 2H), 2.36-2.23 (m, 1H), 2.16 (s, 6H), 1.84-1.72 (m, 2H), 1.48-1.37 (m, 2H), 0.85 (t, J=8.0 Hz, 2H), −0.03 (s, 9H).

### Intermediate A12: 4-(4-methoxybenzyl)-3,5-bis(phenylsulfonyl)-4H-1,2,4-triazole

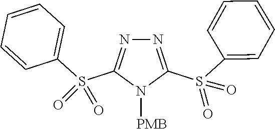

### Step A: 3,5-dibromo-4-(4-methoxybenzyl)-4H-1,2,4-triazole and 3,5-dibromo-1-(4-methoxybenzyl)-1H-1,2,4-triazole

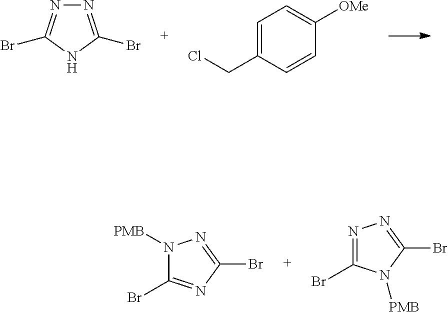

To a stirred solution of 3,5-dibromo-4H-1,2,4-triazole (20.0 g, 88 mmol) and DIPEA (30.7 mL, 176 mmol) in MeCN (400 mL) was added 1-(chloromethyl)-4-methoxy-benzene (11.90 mL, 88 mmol) followed by KI (7.32 g, 44.1 mmol) and the reaction was heated at 80° C. overnight. After cooling, the reaction was concentrated in vacuo and the residue was partitioned between EtOAc (100 mL) and water (100 mL). The aqueous layer was extracted with EtOAc (2×100 mL) and the combined organics were washed with brine (200 mL), dried (MgSO₄) and concentrated in vacuo. The product was purified by chromatography on silica gel (330 g, 0-60% EtOAc/isohexane) to afford 3,5-dibromo-1-(4-methoxybenzyl)-1H-1,2,4-triazole (21.5 g, 70%) as an off-white solid and 3,5-dibromo-4-(4-methoxybenzyl)-4H-1,2,4-triazole (2.74 g, 6%, 66% purity by ¹H NMR) as an off-white solid.

3,5-Dibromo-1-(4-methoxybenzyl)-1H-1,2,4-triazole: ¹H NMR δ 7.28-7.21 (m, 2H), 6.99-6.92 (m, 2H), 5.33 (s, 2H), 3.75 (s, 3H).

3,5-Dibromo-4-(4-methoxybenzyl)-4H-1,2,4-triazole: ¹H NMR δ 7.18-7.11 (m, 2H), 7.00-6.92 (m, 2H), 5.19 (s, 2H), 3.75 (d, J=1.2 Hz, 3H).

### Step B: 4-(4-methoxybenzyl)-3,5-bis(phenylthio)-4H-1,2,4-triazole

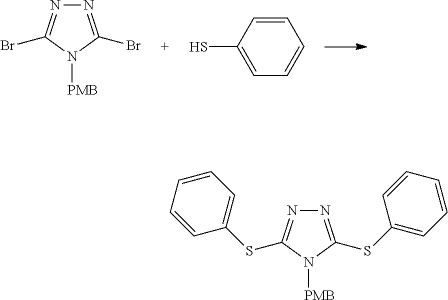

A mixture of 3,5-dibromo-4-(4-methoxybenzyl)-4H-1,2,4-triazole (66% purity by 1H NMR, 2.5 g, 4.75 mmol), thiophenol (2 mL, 19.42 mmol) and DBU (3.6 mL, 23.88 mmol) in dioxane (30 mL) was heated at 6° C. for 8 h. After cooling, the mixture was partitioned between TBME (150 mL) and water (50 mL). The organic layer was washed with aq 2 M NaOH (30 mL), brine (30 mL), dried (MgSO₄) and evaporated. The crude product was purified by chromatography on silica gel (80 g column, 0-50% EtOAc/isohexane) to afford the title compound (1.85 g, 91%) as a white solid.

LCMS m/z 405.9 (M+H)⁺ (ES⁺).

¹H NMR (CDCl₃) δ 7.36-7.25 (m, 10H), 6.89 (d, J=8.7 Hz, 2H), 7.70 (d, J=8.7 Hz, 2H), 5.14 (s, 2H), 3.77 (s, 3H).

### Step C: 4-(4-methoxybenzyl)-3,5-bis(phenylsulfonyl)-4H-1,2,4-triazole

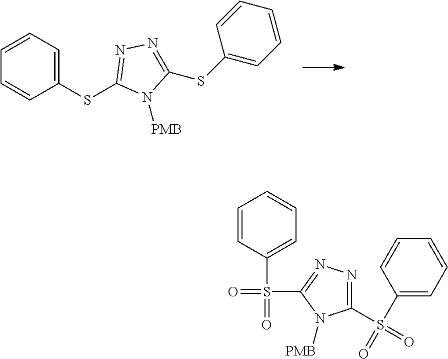

m-CPBA (4.6 g, 20.53 mmol) was added portionwise over 5 min to a solution of 4-(4-methoxybenzyl)-3,5-bis(phenylthio)-4H-1,2,4-triazole (1.83 g, 4.51 mmol) in DCM (40 mL) at RT. The reaction was stirred for 24 h, partitioned between 10% aq Na₂S₂O₃ (30 mL) and DCM (100 mL). The organic layer washed with sat aq NaHCO₃ (30 mL), brine (30 mL), dried (MgSO₄) and evaporated. The crude product was purified by chromatography on silica gel (80 g column, 0-40% EtOAc/isohexane) to afford the title compound (1.58 g, 74%) as a white solid.

LCMS m/z 469.85 (M+H)⁺ (ES⁺).

¹H NMR (CDCl₃) δ 7.89-7.84 (m, 4H), 7.71-7.65 (m, 2H), 7.53-7.47 (m, 4H), 7.08-7.04 (m 2H), 6.82-6.78 (m 2H), 5.99 (s, 2H), 3.83 (s, 3H).

### Intermediate A3:2-((3-bromo-1-((2-(trimethylsilyl)ethoxy)methyl)-1H-1,2,4-triazol-5-yl)sulfonyl)-6-methyl-2,6-diazaspiro[3.4]octane

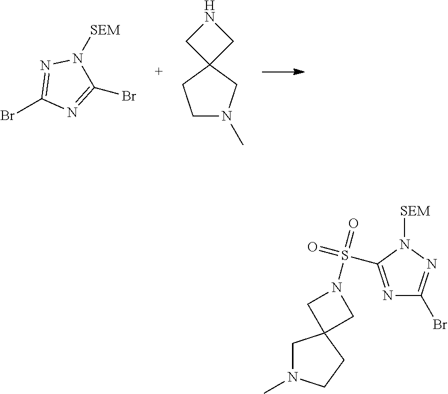

Prepared according to the general procedure of 3-bromo-N-(2-(diethylamino)ethyl)-1-((2-(trimethylsilyl)ethoxy)methyl)-1H-1,2,4-triazole-5-sulfonamide (Intermediate A9) from 3,5-dibromo-1-((2-(trimethylsilyl)ethoxy)methyl)-1H-1,2,4-triazole and 6-methyl-2,6-diazaspiro[3.4]octane (Intermediate X17) to afford the title compound (0.840 g, 32%) as a brown oil.

LCMS m/z 466.2/468.2 (M+H)⁺ (ES⁺).

¹H NMR (DMSO-d₆) δ 5.72 (s, 2H), 4.20 (d, J=8.2 Hz, 2H), 4.16 (d, J=8.2 Hz, 2H), 3.77-3.65 (m, 2H), 2.77 (s, 2H), 2.59 (t, J=7.2 Hz, 2H), 2.37 (s, 3H), 2.12 (t, J=7.1 Hz, 2H), 0.98-0.89 (m, 2H), −0.00 (s, 9H).

### Intermediate A14:7-((3-bromo-1-((2-(trimethylsilyl)ethoxy)methyl)-1H-1,2,4-triazol-5-yl)sulfonyl)-1-methyl-1,7-diazaspiro[3.5]nonane

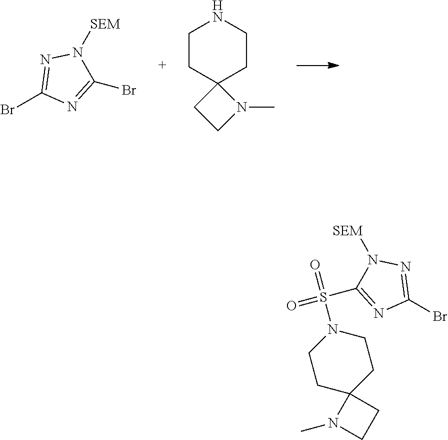

Prepared according to the general procedure of 3-bromo-N-(2-(diethylamino)ethyl)-1-((2-(trimethylsilyl)ethoxy)methyl)-1H-1,2,4-triazole-5-sulfonamide (Intermediate A9) from 3,5-dibromo-1-((2-(trimethylsilyl)ethoxy)methyl)-1H-1,2,4-triazole and 1-methyl-1,7-diazaspiro[3.5]nonane (Intermediate X20) to afford the title compound (0.290 g, 15%) as an orange oil.

LCMS m/z 480.2/482.2 (M+H)⁺ (ES⁺).

¹H NMR (CDCl₃) δ 5.72 (s, 2H), 3.94-3.90 (m, 2H), 3.74-3.65 (m, 2H), 3.30 (s, 2H), 3.04 (td, J=12.3, 3.6 Hz, 2H), 2.28 (s, 3H), 2.01 (t, J=7.1 Hz, 2H), 1.94-1.77 (m, 4H), 0.98-0.89 (m 2H), 0.00 (s, 9H).

### Intermediate A15: 8-((3-bromo-1-((2-(trimethylsilyl)ethoxy)methyl)-1H-1,2,4-triazol-5-yl)sulfonyl)-1-methyl-1,8-diazaspiro[4.5]decane

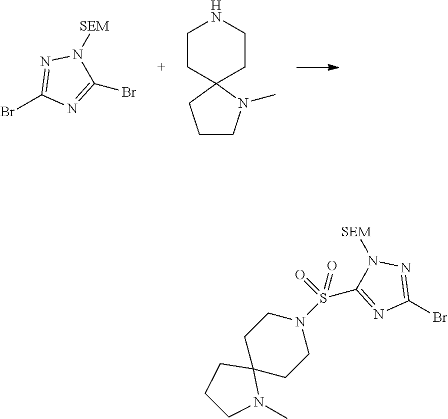 [SMILES: C[Si](C)(C)CCOCn1nc(Br)nc1Br ; CN1CCCC12CCNCC2 ; CN1CCCC12CCN(S(=O)(=O)c1nc(Br)nn1COCC[Si](C)(C)C)CC2]

Prepared according to the general procedure of 3-bromo-N-(2-(diethylamino)ethyl)-1-((2-(trimethylsilyl)ethoxy)methyl)-1H-1,2,4-triazole-5-sulfonamide (Intermediate A9) from 3,5-dibromo-1-((2-(trimethylsilyl)ethoxy)methyl)-1H-1,2,4-triazole and 1-methyl-1,8-diazaspiro[4.5]decane (Intermediate X19) to afford the title compound (0.15 g, 18%) as an orange oil.

LCMS m/z 494.2/496.2 (M+H)⁺ (ES⁺).

¹H NMR (CDCl₃) δ 5.73 (s, 2H), 4.05-3.94 (m, 2H), 3.75-3.67 (m, 2H), 3.09 (td, J=12.9, 2.5 Hz, 2H), 2.83 (s, 2H), 2.33 (s, 3H), 1.95-1.68 (m, 6H), 1.39 (d, J=12.8 Hz, 2H), 1.02-0.81 (m, 2H), 0.00 (s, 9H).

### Intermediate A16: 3-bromo-5-(methylsulfonyl)-1-((2-(trimethylsilyl)ethoxy)-methyl)-1H-1,2,4-triazole

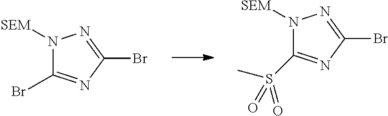 [SMILES: C[Si](C)(C)CCOCn1nc(Br)nc1Br ; C[Si](C)(C)CCOCn1nc(Br)nc1S(C)(=O)=O]

Sodium thiomethoxide (0.756 g, 10.8 mmol) was added portion wise to a solution of 3,5-dibromo-1-((2-(trimethylsilyl)ethoxy)methyl)-1H-1,2,4-triazole (3.5 g, 9.80 mmol) in DMF (25 mL) and the reaction was stirred at 85° C. for 3 h. The mixture was diluted with EtOAc (100 mL) and washed with water/brine (2:1, 3×150 mL). The organic phase was separated, dried (MgSO₄) and concentrated in vacuo. The residue was taken up in DCM (30 mL), m-CPBA (5.07 g, 29.4 mmol) was added portion wise at 0° C. and the reaction stirred at RT for 18 h. The reaction mixture was diluted with DCM (100 mL) and washed with sat aq Na₂SO₃ (5×250 mL). The organic phase was separated, washed with sat aq NaHCO₃ (30 mL), dried (MgSO₄) and concentrated in vacuo to afford the title compound (2.73 g, 77%) as a colourless oil.

¹H NMR (DMSO-d₆) δ 5.75 (s, 2H), 3.71-3.62 (m, 2H), 3.57 (s, 3H), 0.93-0.72 (m, 2H), −0.04 (s, 9H).

### Intermediate A17: benzyl ((3-((1,2,3,5,6,7-hexahydro-s-indacen-4-yl)amino)-1-((2-(trimethylsilyl)ethoxy)methyl)-1H-1,2,4-triazol-5-yl)(oxo)(phenyl)-6-sulfaneylidene)carbamate

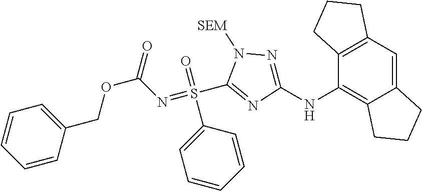 [SMILES: C[Si](C)(C)CCOCn1nc(Nc2c3c(cc4c2CCC4)CCC3)nc1S(=O)(=NC(=O)OCc1ccccc1)c1ccccc1]

### Step A: 3-bromo-5-(phenylsulfonimidoyl)-1-((2-(trimethylsilyl)ethoxy)methyl)-1H-1,2,4-triazole

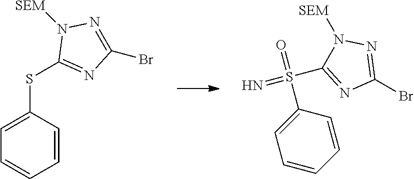 [SMILES: C[Si](C)(C)CCOCn1nc(Br)nc1Sc1ccccc1 ; C[Si](C)(C)CCOCn1nc(Br)nc1S(=N)(=O)c1ccccc1]

Ammonium carbamate (450 mg, 5.76 mmol) followed by PhI(OAc)₂ (1.25 g, 3.88 mmol) was added to a stirred solution of 3-bromo-5-(phenylthio)-1-((2-(trimethylsilyl)-ethoxy)methyl)-1H-1,2,4-triazole (Intermediate A1, Step A) (0.5 g, 1.294 mmol) in MeOH (15 mL) at RT for 30 min. Addition of further ammonium carbamate (450 mg, 5.76 mmol) and PhI(OAc)₂ (1.250 g, 3.88 mmol) was repeated twice more then MeOH (50 mL) and isohexane (50 mL) were added. The MeOH layer was separated, evaporated and partitioned between EtOAc (50 mL) and brine (20 mL). The organic layer was dried (MgSO₄), filtered, evaporated and the residue was purified by chromatography on silica gel (40 g cartridge, 0-30% EtOAc/isohexane) to afford the title compound (240 mg, 42%) as an oil.

LCMS m/z 416.8/418.8 (M+H)⁺ (ES⁺).

¹H NMR (CDCl₃) δ 8.26-8.21 (m, 2H), 7.72-7.68 (m, 1H), 7.62-7.57 (m, 2H), 5.96 (d, J=10.5 Hz, 1H), 5.75 (d, J=10.5 Hz, 1H), 3.64-3.53 (m, 2H), 0.91-0.76 (m, 2H), 0.00 (s, 9H). One exchangeable proton not observed.

### Step B: benzyl((3-bromo-1-((2-(trimethylsilyl)ethoxy)methyl)-1H-1,2,4-triazol-5-yl)(oxo)(phenyl)-λ⁶-sulfaneylidene)carbamate

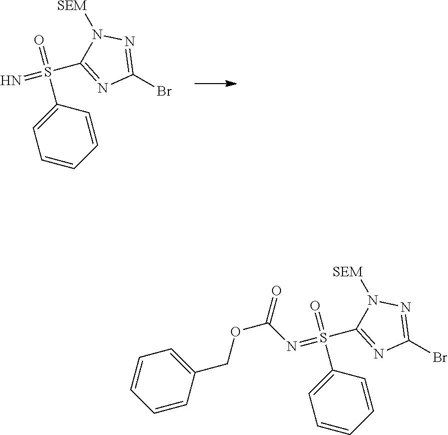 [SMILES: C[Si](C)(C)CCOCn1nc(Br)nc1S(=N)(=O)c1ccccc1 ; C[Si](C)(C)CCOCn1nc(Br)nc1S(=O)(=NC(=O)OCc1ccccc1)c1ccccc1]

Benzyl chloroformate (90 L, 0.638 mmol) was added to a solution of 3-bromo-5-(phenylsulfonimidoyl)-1-((2-(trimethylsilyl)ethoxy)methyl)-1H-1,2,4-triazole (225 mg, 0.539 mmol) and pyridine (70 μL, 0.865 mmol) in DCM (3 mL) at RT. The mixture was stirred for 2 h, cooled in an ice bath, then pyridine (2000 L) followed by benzyl chloroformate (90 L) added. The mixture was warmed to RT stirred for 20 h then partitioned between DCM (20 mL) and water (20 mL). The organic layer was washed with aq. 1M HCl (20 mL), brine (20 mL), dried (MgSO₄), filtered and evaporated. The crude product was purified by chromatography on silica gel (24 g cartridge, 0-30% EtOAc/isohexane) to afford the title compound (142 mg, 45%) as a clear oil.

¹H NMR (CDCl₃) δ 8.28-8.23 (m, 2H), 7.79-7.73 (m, 1H), 7.66-7.60 (m, 2H), 7.40-7.31 (m, 5H), 6.18 (d, J=10.5 Hz, 1H), 5.52 (d, J=10.6 Hz, 1H), 5.21-5.07 (m, 2H), 3.74-3.63 (m, 2H), 1.00-0.83 (m, 2H), 0.02 (s, 9H).

### Step C: benzyl ((3-((1,2,3,5,6,7-hexahydro-s-indacen-4-yl)amino)-1-((2-(trimethylsilyl)ethoxy)methyl)-1H-1,2,4-triazol-5-yl)(oxo)(phenyl)-λ⁶-sulfaneylidene)carbamate

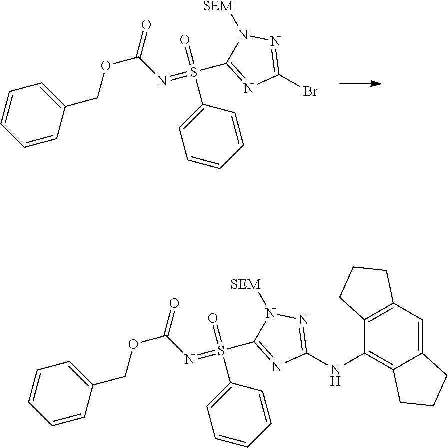 [SMILES: C[Si](C)(C)CCOCn1nc(Br)nc1S(=O)(=NC(=O)OCc1ccccc1)c1ccccc1 ; C[Si](C)(C)CCOCn1nc(Nc2c3c(cc4c2CCC4)CCC3)nc1S(=O)(=NC(=O)OCc1ccccc1)c1ccccc1]

Nitrogen gas was bubbled through a mixture of benzyl ((3-bromo-1-((2-(trimethylsilyl)-ethoxy)methyl)-1H-1,2,4-triazol-5-yl)(oxo)(phenyl)-λ⁶-sulfaneylidene)carbamate (130 mg, 0.236 mmol), 1,2,3,5,6,7-hexahydro-s-indacen-4-amine (45 mg, 0.260 mmol), K₂CO₃ (98 mg, 0.707 mmol) and Pd-175 (25 mg, 0.032 mmol) in dioxane (4 mL) for 15 min then the mixture was heated at 80° C. for 4 h. The mixture was cooled, TBME (20 mL) was added, filtered and evaporated. The crude product was purified by chromatography on silica gel (24 g cartridge, 0-30% EtOAc/isohexane) to afford the title compound (98 mg, 61%) as a solid.

¹H NMR (CDCl₃) δ 8.27-8.23 (m, 2H), 7.76-7.71 (m, 1H), 7.63-7.58 (m, 2H), 7.36-7.30 (m, 5H), 6.95 (s, 1H), 6.03 (d, J=10.6 Hz, 1H), 5.91 (s, 1H), 5.44 (d, J=10.7 Hz, 1H), 5.20-5.10 (m, 2H), 3.71-3.60 (m, 2H), 2.88 (t, J=7.4 Hz, 4H), 2.74-2.58 (m, 4H), 2.06-1.97 (m, 4H), 0.98-0.84 (m, 2H), 0.00 (s, 9H).

### Intermediate B1: 3-bromo-N-(4-fluoro-2,6-diisopropylphenyl)-1-((2-(trimethylsilyl)ethoxy)methyl)-1H-1,2,4-triazol-5-amine

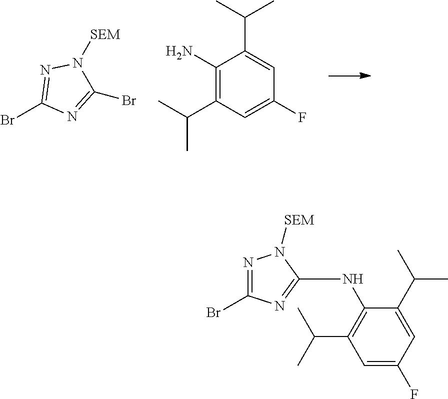

To an ice-cooled solution of 3,5-dibromo-1-((2-(trimethylsilyl)ethoxy)methyl)-1H-1,2,4-triazole (2.00 g, 5.60 mmol) and 4-fluoro-2,6-diisopropylaniline (2.20 g, 11.3 mmol) in THF (20 mL) was added dropwise LiHMDS (1 M in THF, 11.0 mL, 11.0 mmol). The reaction mixture was stirred at RT overnight and quenched by slow addition of sat aq NH₄Cl (20 mL). The product was extracted with DCM (2×20 mL) and the organics were dried (phase separator) and concentrated in vacuo. The crude product was purified by chromatography on silica gel (80 g column, 0-20% EtOAc/isohexane) to afford the title compound (1.82 g, 68% yield) as a pale orange solid.

¹H NMR (DMSO-d₆) δ 8.61 (s, 1H), 7.01 (d, J=9.9 Hz, 2H), 5.41 (s, 2H), 3.67-3.60 (m, 2H), 3.14-3.03 (m, 2H), 1.12-1.07 (m, 12H), 0.92-0.85 (m, 2H), 0.00 (s, 9H).

### Intermediate B2: sodium 5-((1,2,3,5,6,7-hexahydro-s-indacen-4-yl)amino)-1-((2-(trimethylsilyl)ethoxy)methyl)-1H-1,2,4-triazole-3-sulfinate

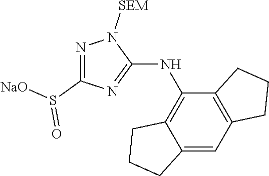

### Step A: 3-bromo-N-(1,2,3,5,6,7-hexahydro-s-indacen-4-yl)-1-((2-(trimethylsilyl)ethoxy)methyl)-1H-1,2,4-triazol-5-amine

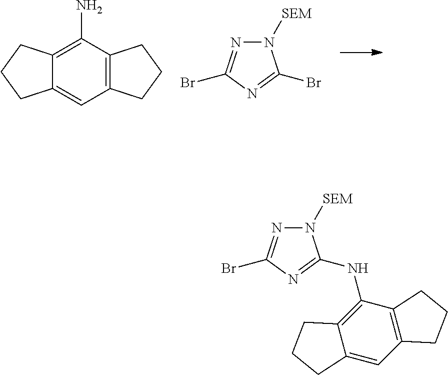

LiHMDS (1 M in THF, 200 mL, 200 mmol) (cooled by ice bath for 10 min prior to addition) was added to an ice-cooled solution of 1,2,3,5,6,7-hexahydro-s-indacen-4-amine (19.1 g, 110 mmol) and 3,5-dibromo-1-((2-(trimethylsilyl)ethoxy)methyl)-1H-1,2,4-triazole (35.7 g, 100 mmol) in THF (200 mL). The reaction mixture was stirred at RT for 2 h, quenched using aq 1 M HCl (100 mL) and washed with additional aq 1 M HCl (100 mL). The organic extract was dried (phase separator) and concentrated in vacuo. The residue was dissolved in hot EtOAc (100 mL) and hexane (600 mL) was added, the mixture was cooled to 0° C. and the resultant precipitate was collected by filtration, washing with isohexane to afford the title compound (32.5 g, 72% yield) as a grey solid.

¹H NMR (DMSO-d₆) δ 8.68 (s, 1H), 6.97 (s, 1H), 5.37 (s, 2H), 3.68-3.53 (m, 2H), 2.83 (t, J=7.4 Hz, 4H), 2.64 (t, J=7.4 Hz, 4H), 1.97 (p, J=7.4 Hz, 4H), 0.88 (t, J=7.5 Hz, 2H), −0.02 (s, 9H).

### Step B: methyl 3-((5-((1,2,3,5,6,7-hexahydro-s-indacen-4-yl)amino)-1-((2-(trimethylsilyl)ethoxy)methyl)-1H-1,2,4-triazol-3-yl)thio)propanoate

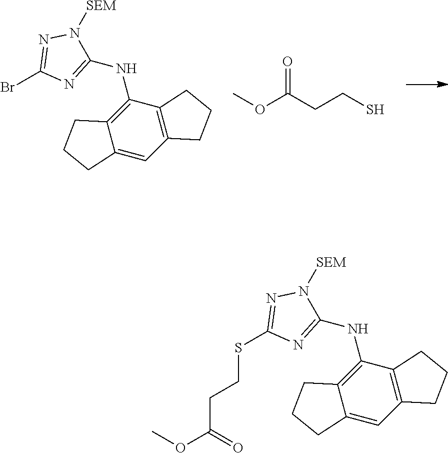

Pd-175 (0.174 g, 0.222 mmol), KO^{t}Bu (0.5 g, 4.46 mmol) and 3-bromo-N-(1,2,3,5,6,7-hexahydro-s-indacen-4-yl)-1-((2-(trimethylsilyl)ethoxy)methyl)-1H-1,2,4-triazol-5-amine (1.00 g, 2.225 mmol) were charged to a reaction vessel under N₂. A solution of methyl 3-mercaptopropanoate (0.493 mL, 4.45 mmol) in 1,4-dioxane (10 mL) was added and the reaction was degassed with N₂ for 10 min before being heated at 75° C. for 18 h. The reaction mixture was diluted with EtOAc (100 mL), washed with water (100 mL) and sat aq NH₄C (100 mL). The organic phase was separated, dried (MgSO₄) and loaded onto silica. The crude was purified by chromatography on silica gel (80 g column, 0-20% EtOAc/isohexane) to afford the title compound (875 mg, 78% yield) as a colourless solid.

LCMS m/z 489.5 (M+H)⁺ (ES⁺); 487.3 (M−H)⁻ (ES⁻).

¹H NMR (DMSO-d₆) δ 8.42 (s, 1H), 6.94 (s, 1H), 5.35 (s, 2H), 3.65-3.55 (m, 5H), 3.12 (t, J=7.0 Hz, 2H), 2.82 (t, J=7.4 Hz, 4H), 2.73 (t, J=7.1 Hz, 2H), 2.64 (t, J=7.3 Hz, 4H), 1.96 (p, J=7.4 Hz, 4H), 0.93-0.82 (m, 2H), −0.02 (s, 9H).

### Step C: methyl 3-((5-((1,2,3,5,6,7-hexahydro-s-indacen-4-yl)amino)-1-((2-(trimethylsilyl)ethoxy)methyl)-1H-1,2,4-triazol-3-yl)sulfonyl)propanoate

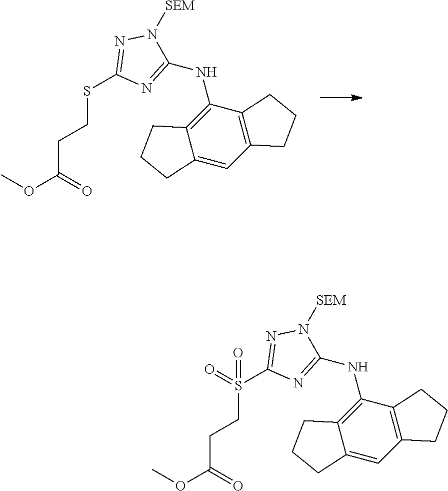 [SMILES: COC(=O)CCSc1nc(Nc2c3c(cc4c2CCC4)CCC3)n(COCC[Si](C)(C)C)n1 ; COC(=O)CCS(=O)(=O)c1nc(Nc2c3c(cc4c2CCC4)CCC3)n(COCC[Si](C)(C)C)n1]

m-CPBA (883 mg, 3.94 mmol) was added to a solution of methyl 3-((5-((1,2,3,5,6,7-hexahydro-s-indacen-4-yl)amino)-1-((2-(trimethylsilyl)ethoxy)methyl)-1H-1,2,4-triazol-3-yl)thio)propanoate (770 mg, 1.58 mmol) in DCM (10 mL) at 0° C. The mixture was stirred for 15 min at 0° C. and then stirred for 3 h at RT. The reaction was quenched with aq sodium sulfite and the layers were separated. The aqueous was extracted with DCM (2×25 mL). The organics were combined and washed with sat aq NaHCO₃ (2×20 mL), dried (MgSO₄) and concentrated in vacuo to afford the title compound (818 mg, 92% yield) as a yellow solid.

LCMS m/z 521.4 (M+H)⁺ (ES⁺); 519.0 (M−H)⁻ (ES⁻).

¹H NMR (DMSO-d₆) δ 8.93 (s, 1H), 7.00 (s, 1H), 5.53 (s, 2H), 3.67-3.59 (m, 2H), 3.61-3.53 (m, 5H), 2.84 (t, J=7.4 Hz, 4H), 2.71 (t, J=7.3 Hz, 2H), 2.65 (t, J=7.3 Hz, 4H), 1.97 (p, J=7.4 Hz, 4H), 0.93-0.86 (m, 2H), −0.02 (s, 9H).

### Step D: sodium 5-((1,2,3,5,6,7-hexahydro-s-indacen-4-yl)amino)-1-((2-(trimethylsilyl)ethoxy)methyl)-1H-1,2,4-triazole-3-sulfinate

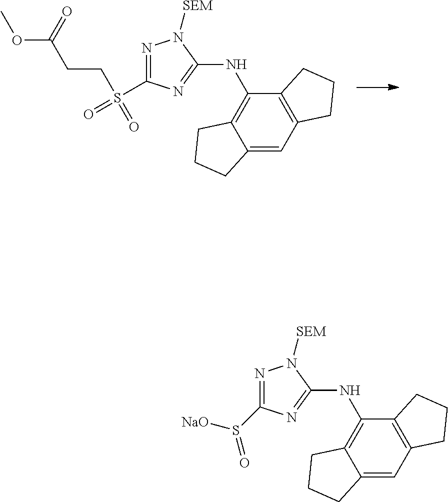

To a solution of methyl 3-((5-((1,2,3,5,6,7-hexahydro-s-indacen-4-yl)amino)-1-((2-(trimethylsilyl)ethoxy)methyl)-1H-1,2,4-triazol-3-yl)sulfonyl)propanoate (610 mg, 1.171 mmol) in THF (4 mL) was added NaOMe (5.4 M in THF, 0.22 mL, 1.17 mmol) and MeOH (0.7 mL). The reaction mixture was stirred for 1 h. The resultant precipitate was collected by filtration, washing with THF (20 mL) to afford the title compound (391 mg, 69% yield) as a yellow solid.

LCMS m/z 371.3 (M-SO₂+2H)+(ES⁺); 369.1 (M−SO₂)⁻ (ES⁻).

¹H NMR (DMSO-d₆) δ 7.98 (s, 1H), 6.90 (s, 1H), 5.33 (s, 2H), 3.61-3.54 (m, 2H), 2.82 (t, J=7.5 Hz, 4H), 2.64 (t, J=7.3 Hz, 4H), 1.96 (p, J=7.5 Hz, 4H), 0.93-0.84 (m, 2H), −0.01 (s, 9H).

### Intermediate B1: 3-((5-((4-fluoro-2-isopropyl-6-(2-methoxypyridin-4-yl)phenyl)amino)-1-((2-(trimethylsilyl)ethoxy)methyl)-1H-1,2,4-triazol-3-yl)sulfonyl)propan-1-ol

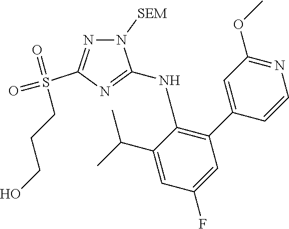 [SMILES: COc1cc(-c2cc(F)cc(C(C)C)c2Nc2nc(S(=O)(=O)CCCO)nn2COCC[Si](C)(C)C)ccn1]

### Step A: 3-bromo-N-(4-fluoro-2-isopropyl-6-(2-methoxypyridin-4-yl)phenyl)-1-((2-(trimethylsilyl)ethoxy)methyl)-1H-1,2,4-triazol-5-amine

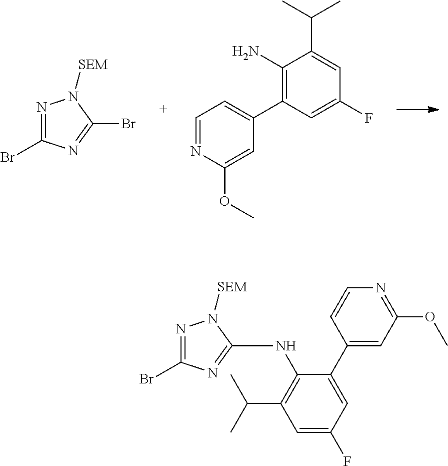 [SMILES: C[Si](C)(C)CCOCn1nc(Br)nc1Br ; COc1cc(-c2cc(F)cc(C(C)C)c2N)ccn1 ; COc1cc(-c2cc(F)cc(C(C)C)c2Nc2nc(Br)nn2COCC[Si](C)(C)C)ccn1]

Prepared according to the general procedure of 3-bromo-N-(4-fluoro-2,6-diisopropylphenyl)-1-((2-(trimethylsilyl)ethoxy)methyl)-1H-1,2,4-triazol-5-amine (Intermediate B1) from 4-fluoro-2-isopropyl-6-(2-methoxypyridin-4-yl)aniline (Intermediate C2) and 3,5-dibromo-1-((2-(trimethylsilyl)ethoxy)methyl)-1H-1,2,4-triazole to afford the title compound (1.74 g, 64% yield) as a white solid.

LCMS m/z 536.3, 538.3 (M+H)⁺ (ES⁺); 534.2, 536.1 (M−H)⁻ (ES⁻).

¹H NMR (DMSO-d₆) δ 8.70 (s, 1H), 8.1 (d, J=5.2 Hz, 1H), 7.31 (dd, J=9.9, 3.0 Hz, 1H), 7.09 (dd, J=8.8, 3.0 Hz, 1H), 6.91 (dd, J=5.3, 1.4 Hz, 1H), 6.76 (app s, 1H), 5.21 (s, 2H), 3.82 (s, 3H), 3.45 (t, J=8.0 Hz, 2H), 3.10 (sept, J=7.1 Hz, 1H), 1.14 (d, J=6.8 Hz, 6H), 0.79 (t, J=8.0 Hz, 2H), −0.03 (s, 9H).

### Step B: 3-((5-((4-fluoro-2-isopropyl-6-(2-methoxypyridin-4-yl)phenyl)amino)-1-((2-(trimethylsilyl)ethoxy)methyl)-1H-1,2,4-triazol-3-yl)thio)propan-1-ol

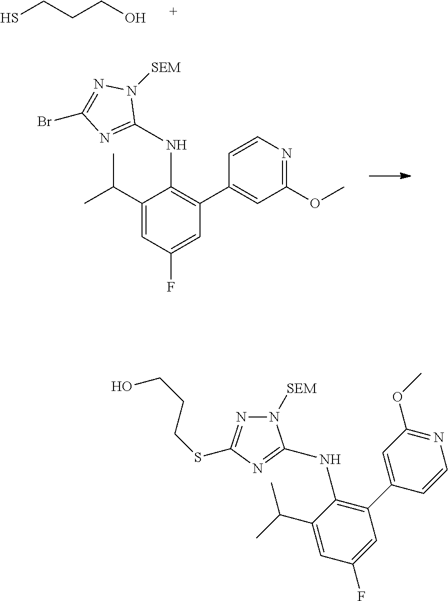 [SMILES: OCCCS ; COc1cc(-c2cc(F)cc(C(C)C)c2Nc2nc(Br)nn2COCC[Si](C)(C)C)ccn1 ; COc1cc(-c2cc(F)cc(C(C)C)c2Nc2nc(SCCCO)nn2COCC[Si](C)(C)C)ccn1]

1,4-Dioxane (50 mL), DIPEA (0.889 mL, 5.09 mmol) and 3-mercaptopropan-1-ol (0.440 mL, 5.09 mmol) were added to 3-bromo-N-(4-fluoro-2-isopropyl-6-(2-methoxypyridin-4-yl)phenyl)-1-((2-(trimethylsilyl)ethoxy)methyl)-1H-1,2,4-triazol-5-amine (1.74 g, 3.24 mmol), DIPEA (0.889 mL, 5.09 mmol) and Pd(dba)₂ (234 mg, 0.407 mmol) under N₂. The reaction was deoxygenated with a stream of N₂ for 10 min before being stirred at 100° C. for 16 h. After cooling to RT, the reaction was diluted with EtOAc (150 mL) and washed with brine (100 mL). The aqueous phase was extracted with EtOAc (100 mL) and the combined organics dried (MgSO₄) and concentrated in vacuo. The product was purified by chromatography on silica gel (120 g column, 0-100% EtOAc/isohexane) to afford the title compound (1.59 g, 79% yield) as an orange gum.

LCMS m/z 548.4 (M+H)⁺ (ES⁺); 545.9 (M−H)⁻ (ES⁻).

¹H NMR (DMSO-d₆) δ 8.40 (s, 1H), 8.08 (dd, J=5.3, 0.8 Hz, 1H), 7.29 (dd, J=10.0, 3.0 Hz, 1H), 7.08 (dd, J=8.8, 3.0 Hz, 1H), 6.93 (dd, J=5.3, 1.4 Hz, 1H), 6.79-6.74 (m, 1H), 5.19 (s, 2H), 4.47 (t, J=5.2 Hz, 1H), 3.81 (s, 3H), 3.51-3.37 (m, 4H), 3.23-3.05 (m, 1H), 2.87 (t, J=7.1 Hz, 2H), 1.64 (p, J=6.5 Hz, 2H), 1.13 (d, J=6.8 Hz, 6H), 0.84-0.76 (m, 2H), −0.04 (s, 9H).

### Step C: 3-((5-((4-fluoro-2-isopropyl-6-(2-methoxypyridin-4-yl)phenyl)amino)-1-((2-(trimethylsilyl)ethoxy)methyl)-1H-1,2,4-triazol-3-yl)sulfonyl)propan-1-ol

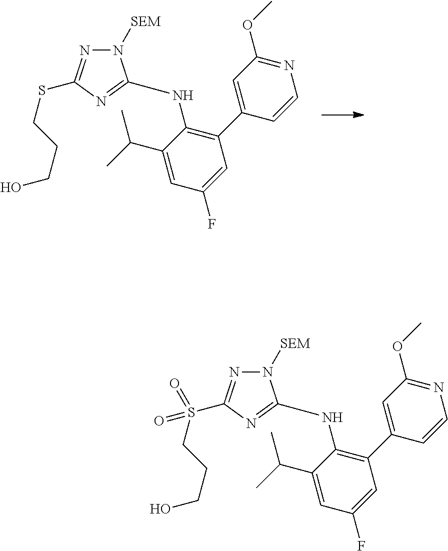 [SMILES: COc1cc(-c2cc(F)cc(C(C)C)c2Nc2nc(SCCCO)nn2COCC[Si](C)(C)C)ccn1 ; COc1cc(-c2cc(F)cc(C(C)C)c2Nc2nc(S(=O)(=O)CCCO)nn2COCC[Si](C)(C)C)ccn1]

m-CPBA (0.62 g, 3.62 mmol) was added to a solution of 3-((5-((4-fluoro-2-isopropyl-6-(2-methoxypyridin-4-yl)phenyl)amino)-1-((2-(trimethylsilyl)ethoxy)methyl)-1H-1,2,4-triazol-3-yl)thio)propan-1-ol (1.59 g, 2.90 mmol) in DCM (20 mL) at 0° C. followed by another portion of m-CPBA (1.13 g, 6.54 mmol) after 45 min. The reaction was stirred at RT for 18 h then quenched with aq Na₂SO₃ (75 mL). The mixture was extracted with DCM (2×100 mL) and the combined organics washed with sat aq NaHCO₃ (100 mL), dried (MgSO4) and concentrated in vacuo. The residue was dissolved in MeCN (2 mL) and MeOH (1 mL) and B₂Pin₂ (0.736 g, 2.90 mmol) was added and the reaction mixture was heated at 50° C. for 6 h. Additional B₂Pin₂ (0.736 g, 2.90 mmol) was added and the reaction was stirred at 50° C. for a further 2 h, then at RT for 16 h. Ethylenediamine (3.92 mL, 58.0 mmol) was added and the reaction stirred at RT for 1 h, diluted with water (10 mL) and extracted with MTBE (3×30 mL). The organic phases were combined, dried (Na₂SO₄), filtered and concentrated in vacuo to the title compound (0.95 g, 54% yield) as a solid.

LCMS m/z 580.5 (M+H)⁺ (ES⁺); 578.3 (M−H)⁻ (ES⁻).

¹H NMR (CDCl₃) δ 8.09 (d, J=5.2 Hz, 1H), 7.10 (dd, J=9.6, 3.0 Hz, 1H), 6.88 (dd, J=8.2, 2.9 Hz, 1H), 6.82 (d, J=5.2 Hz, 1H), 6.68 (s, 1H), 6.25 (s, 1H), 5.31 (s, 2H), 3.90 (s, 3H), 3.71 (t, J=6.0 Hz, 2H), 3.50-3.43 (m, 2H), 3.30 (dd, J=8.5, 6.5 Hz, 2H), 3.05 (sept, J=6.1 Hz, 1H), 2.14 (br s, 1H), 1.97-1.88 (m, 2H), 1.23 (s, 6H), 0.91-0.74 (m, 2H), 0.01 (s, 9H).

### Intermediate B4: 5-((1,2,3,5,6,7-hexahydro-s-indacen-4-yl)amino)-1-((2-(trimethylsilyl)ethoxy)methyl)-1H-1,2,4-triazole-3-sulfonylchloride

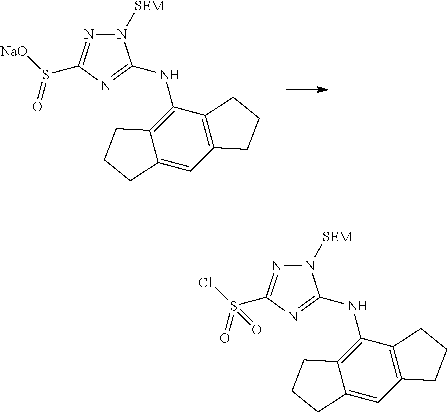

Sodium 5-((1,2,3,5,6,7-hexahydro-s-indacen-4-yl)amino)-1-((2-(trimethylsilyl)ethoxy)-methyl)-1H-1,2,4-triazole-3-sulfinate (Intermediate B2) (0.300 g, 0.657 mmol) was suspended in DCM (4 mL) at 0° C. and then NCS (92 mg, 0.690 mmol) was added and the reaction was stirred for 1 h. The crude was then purified by chromatography on silica gel (24 g column, 0-50% EtOAc/isohexane) to afford the title compound (0.11 g, 32% yield) as a yellow solid.

¹H NMR (CDCl₃) δ 7.02 (s, 1H), 6.55 (s, 1H), 5.51 (s, 2H), 3.73-3.53 (m, 2H), 2.91 (t, J=7.4 Hz, 4H), 2.74 (t, J=7.3 Hz, 4H), 2.09 (p, J=7.4 Hz, 4H), 1.03-0.92 (m, 2H), 0.03 (s, 9H).

### Intermediate B5: 2-bromo-4-fluoro-6-isopropylphenol

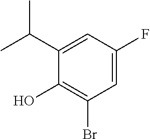

### Step A: 4-fluoro-2-(prop-1-en-2-yl)phenol

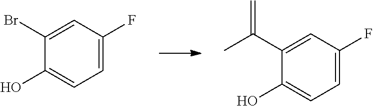

To a mixture of 2-bromo-4-fluorophenol (30 g, 157.07 mmol, 1 eq) and potassium trifluoro(prop-1-en-2-yl)borate (28 g, 188.48 mmol, 1.2 eq) in dioxane (550 mL) and H₂O (110 mL) was added Na₂CO₃ (42 g, 392.68 mmol, 2.5 eq), followed with Pd(dppf)Cl₂ (5.75 g, 7.85 mmol, 0.05 eq) under N₂ atmosphere. Then the mixture was stirred at 90° C. for 4 hours. The mixture was filtered, and the filtrate was diluted with H₂O (1 L) and extracted with EtOAc (2×700 mL). The organic phases were washed with brine (2×1 L), dried over anhydrous Na₂SO₄, filtered and concentrated in vacuum. The residue was purified by silica gel column chromatography (SiO₂, eluting only by petroleum ether) to give the title compound (22 g, 92.1% yield, 82.8% purity on LCMS) as a yellow oil.

¹H NMR (400 MHz, CDCl₃) δ 6.87-6.84 (m, 3H), 5.50 (s, 1H), 5.43 (d, 1H), 5.18 (s, 1H), 2.11 (s, 3H).

LCMS: m/z 151.1 (M−H)⁻ (ES⁻).

### Step B: 4-fluoro-2-isopropylphenol

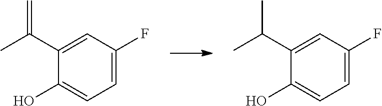

To a solution of 4-fluoro-2-(prop-1-en-2-yl)phenol (22 g, 144.58 mmol, 1 eq) in MeOH (300 mL) was added Pd/C (2.2 g, 10 wt. % loading on activated carbon). Then the mixture was stirred at 25° C. under H₂ atmosphere (20 psi) for 4 hours. The mixture was filtered and the filtrate was concentrated in vacuum. The residue was purified by silica gel column chromatography (SiO₂, petroleum ether:ethyl acetate, 1:0 to 50:1) to give the title compound (22 g, 98.7% yield) as a yellow oil.

¹H NMR (400 MHz, CDCl₃) δ 6.82 (dd, 1H), 6.69-6.64 (m, 1H), 6.62-6.58 (m, 1H), 4.62 (s, 1H), 3.15-3.10 (m, 1H), 1.16 (d, 6H).

LCMS: m/z 153.0 (M−H)⁻ (ES⁻).

### Step C: 2-bromo-4-fluoro-6-isopropylphenol

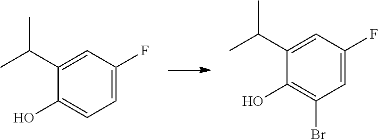

To a solution of 4-fluoro-2-isopropylphenol (20 g, 129.72 mmol, 1 eq) in toluene (200 mL) was added NBS (23.1 g, 129.72 mmol, 1 eq). Then the mixture was stirred at 25° C. for 10 minutes. The mixture was filtered and the filtrate was diluted with H₂O (300 mL) and extracted with EtOAc (2×400 mL). The organic phases were washed with brine (2×500 mL), dried over anhydrous Na₂SO₄, filtered and concentrated in vacuum. The residue was purified by silica gel column chromatography (SiO₂, eluting by only petroleum ether) to give the title compound (23 g, 76.1% yield) as a colourless oil.

¹H NMR (400 MHz, CDCl₃) δ 7.05 (dd, 1H), 6.90 (dd, 1H), 5.40 (s, 1H), 3.35-3.28 (m, 1H), 1.23 (d, 6H).

LCMS: m/z 231.0, 233.0 (M−H)⁻ (ES⁻).

### Intermediate B6: 3,5-dibromo-1-((2-(trimethylsilyl)ethoxy)methyl)-1H-1,2,4-triazole

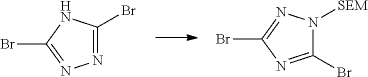 [SMILES: Brc1nnc(Br)[nH]1 ; C[Si](C)(C)CCOCn1nc(Br)nc1Br]

To a mixture of 3,5-dibromo-4H-1,2,4-triazole (50 g, 220.40 mmol, 1 eq) and K₂CO₃ (45.7 g, 330.60 mmol, 1.5 eq) in MeCN (500 mL) was added SEM-Cl (39 g, 233.63 mmol, 1.06 eq) at 25° C. Then the mixture was stirred at 25° C. for 12 hours. The mixture was filtered and the filtrate was concentrated in vacuum. The residue was purified by silica gel column chromatography (SiO₂, petroleum ether:ethyl acetate, 25:1 to 20:1) to give the title compound (60 g, 76.7% yield) as a colourless oil.

¹H NMR (400 MHz, CDCl₃) δ 5.45 (s, 2H), 3.67 (t, 2H), 0.93 (t, 2H), 0.00 (s, 9H).

### Intermediate B7:5-(4-fluoro-2-isopropyl-6-(2-methoxypyridin-4-yl)phenoxy)-1-((2-(trimethylsilyl)ethoxy)methyl)-1H-1,2,4-triazole-3-sulfonylchloride

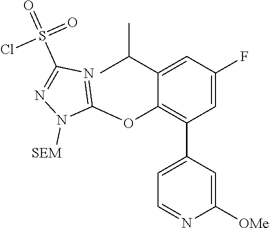 [SMILES: COc1cc(-c2cc(F)cc(C(C)C)c2Oc2nc(S(=O)(=O)Cl)nn2COCC[Si](C)(C)C)ccn1]

### Step A: 4-fluoro-2-isopropyl-6-(2-methoxypyridin-4-yl)phenol

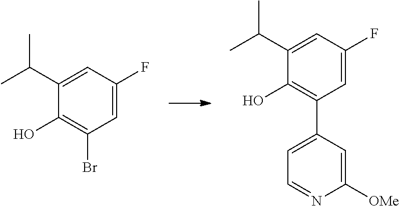 [SMILES: CC(C)c1cc(F)cc(Br)c1O ; COc1cc(-c2cc(F)cc(C(C)C)c2O)ccn1]

To a solution of 2-bromo-4-fluoro-6-isopropylphenol (Intermediate B5) (22 g, 94.39 mmol, 1 eq) and (2-methoxypyridin-4-yl)boronic acid (14.44 g, 94.39 mmol, 1 eq) in dioxane (300 mL) and H₂O (60 mL) was added Na₂CO₃ (25 g, 235.97 mmol, 2.5 eq) and Pd(dppf)Cl₂ (345 g, 4.72 mmol, 0.05 eq) under N₂ atmosphere. Then the mixture was stirred at 90° C. for 2 hours. The mixture was filtered and the filtrate was diluted with H₂O (600 mL) and extracted with EtOAc (2×600 mL). The organic phases were washed with brine (2×600 mL), dried over anhydrous Na₂SO₄, filtered and concentrated in vacuum. The residue was purified by silica gel column chromatography (SiO₂, petroleum ether:ethyl acetate, 50:1 to 10:1) to give the title compound (17 g, 68.9% yield) as a white solid.

¹H NMR (400 MHz, CDCl₃) δ 8.27 (d, 1H), 6.99-6.96 (m, 2H), 6.84 (d, 1H), 6.79 (dd, 1H), 5.13 (s, 1H), 3.99 (s, 3H), 3.34-3.27 (m, 1H), 1.27 (d, 6H).

LCMS: m/z 260.1 (M−H)⁻ (ES⁻).

### Step B: 4-(2-((3-bromo-1-((2-(trimethylsilyl)ethoxy)methyl)-1H-1,2,4-triazol-5-yl)oxy)-5-fluoro-3-isopropylphenyl)-2-methoxypyridine

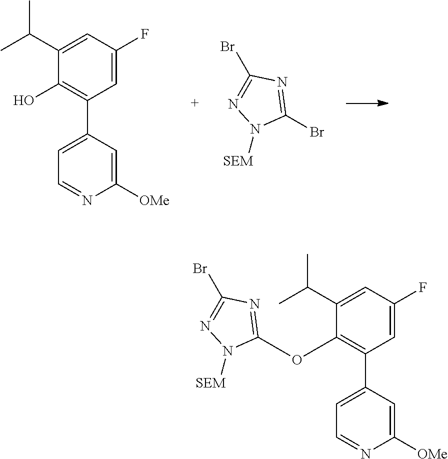 [SMILES: COc1cc(-c2cc(F)cc(C(C)C)c2O)ccn1 ; C[Si](C)(C)CCOCn1nc(Br)nc1Br ; COc1cc(-c2cc(F)cc(C(C)C)c2Oc2nc(Br)nn2COCC[Si](C)(C)C)ccn1]

To a solution of 4-fluoro-2-isopropyl-6-(2-methoxypyridin-4-yl)phenol (5 g, 19-14 mmol, 1 eq) in DMF (70 mL) was slowly added NaH (1.15 g, 28.71 mmol, 60% purity in mineral oil, 1.5 eq) at ° C. Then the mixture was stirred at ° C. for 1 hour. Then to the above solution was added 3,5-dibromo-1-((2-(trimethylsilyl)ethoxy)methyl)-1H-1,2,4-triazole (Intermediate B6) (6.84 g, 9.57 mmol, 0.5 eq) in DMF (10 mL) at 0° C. Then the mixture was stirred at 80° C. for 3 hours. The reaction mixture was quenched with H₂O (300 mL) and extracted with EtOAc (2×300 mL). The organic phases were washed with brine (2×300 mL dried over anhydrous NaSO₄. filtered and concentrated in vacuum. The residue was purified by silica gel column chromatography (SiO₂, petroleum ether:ethyl acetate, 20:1 to 10:1) to give the title compound (7-5 g, 73.1% yield) as a yellow solid.

¹H NMR (400 MHz, CDCl₃) δ 8.12 (d, 1H), 7.11 (dd, 1H), 6.93 (dd, 1H), 6.86 (dd, 1H), 6.74 (s, 1H), 5.20 (s, 2H), 3.93 (s, 3H), 3.60 (t, 2H), 3.08-3.04 (m, 1H), 1.25 (d, 6H), 0.95-0.90 (m, 2H), 0.01 (s, 9H).

LCMS: m/z 537.2, 539.2 (M+H)⁺ (ES⁺).

### Step C: methyl 3-((5-(4-fluoro-2-isopropyl-6-(2-methoxypyridin-4-yl)phenoxy)-1-((2-(trimethylsilyl)ethoxy)methyl)-1H-1,2,4-triazol-3-yl)thio)propanoate

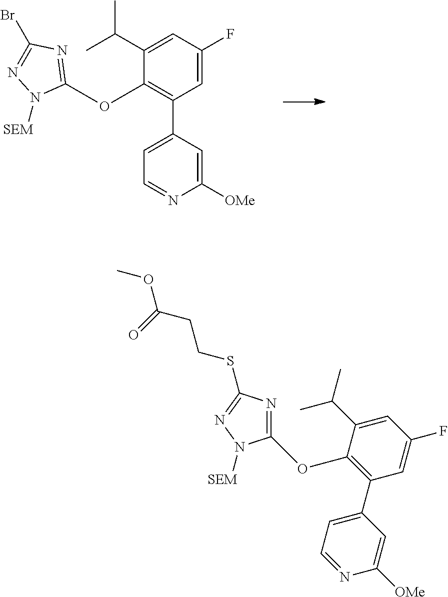 [SMILES: COc1cc(-c2cc(F)cc(C(C)C)c2Oc2nc(Br)nn2COCC[Si](C)(C)C)ccn1 ; COC(=O)CCSc1nc(Oc2c(-c3ccnc(OC)c3)cc(F)cc2C(C)C)n(COCC[Si](C)(C)C)n1]

4-(2-((3-bromo-1-((2-(trimethylsilyl)ethoxy)methyl)-1H-1,2,4-triazol-5-yl)oxy)-5-fluoro-3-isopropylphenyl)-2-methoxypyridine (6.5 g, 12.13 mmol, 1 eq) was dissolved in dioxane (100 mL) and the resulting solution was degassed with N₂ for 10 minutes. To the above solution was added methyl 3-mercaptopropanoate (2.91 g, 24.25 mmol, 2 eq), DIPEA (3.13 g, 24.25 mmol, 2 eq), Pd(dba)₂ (1.05 g, 1.82 mmol, 0.15 eq) and XantPhos (1.05 g, 1.82 mmol, 0.15 eq). The mixture was degassed in vacuum and purged with N₂, and then heated to 100° C. and stirred for 16 hours. The reaction mixture was quenched with H₂O (150 mL) and extracted with EtOAc (2×150 mL). The organic phases were washed with brine (2×200 mL), dried over anhydrous Na₂SO₄, filtered and concentrated in vacuum. The residue was purified by silica gel column chromatography (SiO₂, petroleum ether:ethyl acetate, 15:1 to 8:1) to give the title compound (6.84 g, 97.9 yield) as a yellow oil.

¹H NMR (400 MHz, CDCl₃) δ 8.11 (d, 1H), 7.10 (dd, 1H), 6.91 (dd, 1H), 6.85 (dd, 1H), 6.73 (d, 1H), 5.17 (s, 2H), 3.92 (s, 3H), 3.70 (s, 3H), 3.60 (t, 2H), 3.16-3.12 (m, 3H), 2.65 (t, 2H), 1.25 (d, 6H), 0.95-0.90 (m, 2H), 0.01 (s, 9H).

LCMS: m/z 577.4 (M+H)⁺ (ES⁺).

### Step D: methyl 3-((5-(4-fluoro-2-isopropyl-6-(2-methoxypyridin-4-yl)phenoxy)-1-((2-(trimethylsilyl)ethoxy)methyl)-1H-1,2,4-triazol-3-yl)sulfonyl)propanoate

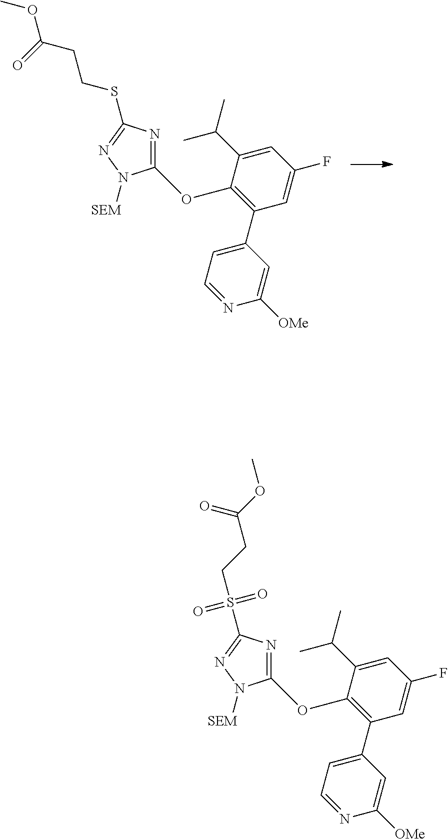 [SMILES: COC(=O)CCSc1nc(Oc2c(-c3ccnc(OC)c3)cc(F)cc2C(C)C)n(COCC[Si](C)(C)C)n1 ; COC(=O)CCS(=O)(=O)c1nc(Oc2c(-c3ccnc(OC)c3)cc(F)cc2C(C)C)n(COCC[Si](C)(C)C)n1]

To a solution of methyl 3-((5-(4-fluoro-2-isopropyl-6-(2-methoxypyridin-4-yl)-phenoxy)-1-((2-(trimethylsilyl)ethoxy)methyl)-1H-1,2,4-triazol-3-yl)thio)propanoate (1 g, 1.74 mmol, 1 eq) in DCM (10 mL) was added m-CPBA (881 mg, 4.34 mmol, 85 wt. % in H₂O, 2.5 eq) at ° C. Then the reaction solution was warmed to 25° C. and stirred for 2 hours. The reaction mixture was quenched with saturated aqueous Na₂SO₃ (30 mL) and extracted with DCM (2×25 mL). The organic phases were washed with Na₂CO₃ (40 mL) and brine (50 mL), dried over anhydrous Na₂SO₄, filtered and concentrated in vacuum. The residue was dissolved in DCM (5 mL), and then to the above solution was added 4,4,5,5-tetramethyl-2-(4,4,5,5-tetramethyl-1,3,2-dioxaborolan-2-yl)-1,3,2-dioxaborolane (1.32 g, 5.21 mmol, 3 eq) at 25° C. The mixture was stirred at 25° C. for 2 hours. Then the mixture was concentrated in vacuum. The residue was purified by silica gel column chromatography (SiO₂, petroleum ether:ethyl acetate, 8:1 to 5:1) to give the title compound (0.9 g, 85.3% yield) as a white solid.

¹H NMR (400 MHz, CDCl₃) δ 8.11 (d, 1H), 7.12 (dd, 1H), 6.93 (dd, 1H), 6.84 (dd, 1H), 6.71 (s, 1H), 5.34 (s, 2H), 3.90 (s, 3H), 3.72 (s, 3H), 3.63 (t, 2H), 3.49-3.43 (m, 2H), 3.11-304 (m, 1H), 2.69 (t, 2H), 1.26 (d, 6H), 0.95-0.90 (m, 2H) and 0.01 (s, 9H).

LCMS: m/z 609.4 (M+H)⁺ (ES⁺).

### Step E: sodium 5-(4-fluoro-2-isopropyl-6-(2-methoxypyridin-4-yl)phenoxy)-1-((2-(trimethylsilyl)ethoxy)methyl)-1H-1,2,4-triazole-3-sulfinate

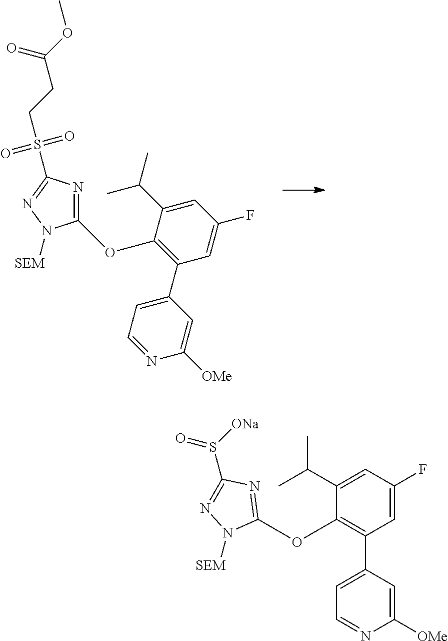

To a solution of methyl 3-((5-(4-fluoro-2-isopropyl-6-(2-methoxypyridin-4-yl)-phenoxy)-1-((2-(trimethylsilyl)ethoxy)methyl)-1H-1,2,4-triazol-3-yl)sulfonyl)-propanoate (500 mg, 822.37 μmol, 1 eq) in MeOH (1 mL) and THF (1 mL) was added NaOMe (44 mg, 822.37 μmol, 1 eq) at 0° C. Then the solution was warmed to 25° C. and stirred for 1 hour. Additional NaOMe (44 mg, 822.37 μmol, 1 eq) was added, and then the mixture was stirred at 25° C. for 1 hour. The mixture was concentrated in vacuum to give the title compound (0.5 g, crude) as a white solid.

¹H NMR (DMSO-d₆) δ 8.12 (d, J=5.3 Hz, 1H), 7.41 (dd, J=9.6, 3.1 Hz, 1H), 7.25 (dd, J=8.6, 3.1 Hz, 1H), 7.02 (dd, J=5.3, 1.5 Hz, 1H), 6.82 (s, 1H), 5.22 (s, 2H), 3.80 (s, 3H), 3.56-3.49 (m, 2H), 3.01-2.92 (m, 1H), 1.16 (d, J=6.9 Hz, 6H), 0.89-0.81 (m, 2H), −0.02 (s, 9H).

LCMS: m/z 521.2 (M-Na)⁻ (ES⁻).

### Step F: 5-(4-fluoro-2-isopropyl-6-(2-methoxypyridin-4-yl)phenoxy)-1-((2-(trimethylsilyl)ethoxy)methyl)-1H-1,2,4-triazole-3-sulfonyl chloride

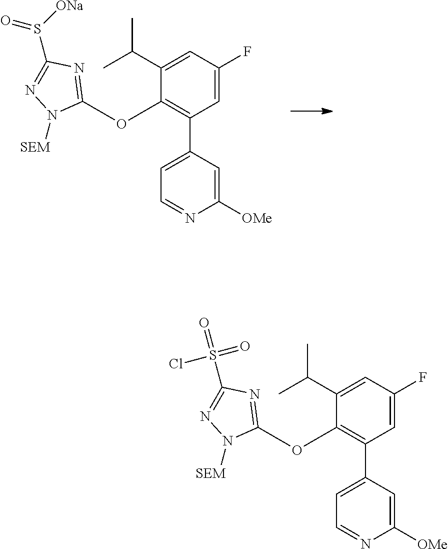

To a mixture of sodium 5-(4-fluoro-2-isopropyl-6-(2-methoxypyridin-4-yl)phenoxy)-1-((2-(trimethylsilyl)ethoxy)methyl)-1H-1,2,4-triazole-3-sulfinate (0.1 g, 183.82 μmol, 1 eq) in DCM (2 mL) was added NCS (37 mg, 275.74 μmol, 1.5 eq). Then the mixture was stirred at 25° C. for 10 minutes. The mixture was quenched with H₂O (4 mL) and extracted with DCM (4 mL). The organic phase was dried over anhydrous Na₂SO₄ and filtered. The filtrate (theoretical amount: 102 mg, crude) in DCM (4 mL) was used in next step directly. An aliquot was quenched with morpholine and analysed by LCMS.

LCMS m/z 608.1 (M+H)⁺ (ES⁺).

### Intermediate B8: 5-(4-fluoro-2-isopropyl-6-(pyridin-4-yl)phenoxy)-1-((2-(trimethylsilyl)ethoxy)methyl)-1H-1,2,4-triazole-3-sulfonyl chloride

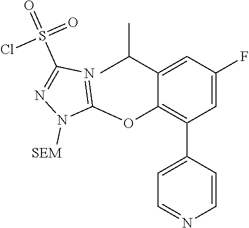 [SMILES: CC(C)c1cc(F)cc(-c2ccncc2)c1Oc1nc(S(=O)(=O)Cl)nn1COCC[Si](C)(C)C]

### Step A: 4-fluoro-2-isopropyl-6-(pyridin-4-yl)phenol

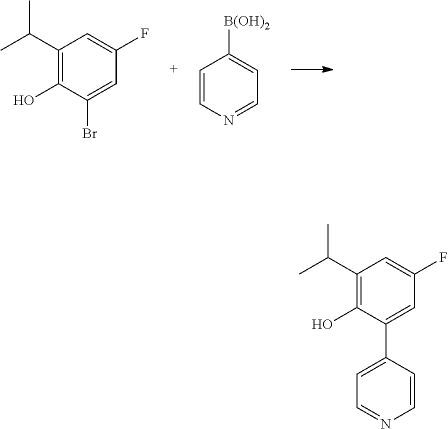 [SMILES: CC(C)c1cc(F)cc(Br)c1O ; OB(O)c1ccncc1 ; CC(C)c1cc(F)cc(-c2ccncc2)c1O]

To a solution of 2-bromo-4-fluoro-6-isopropylphenol (Intermediate B5) (30 g, 128.71 mmol, 1 eq) and pyridin-4-ylboronic acid (16 g, 128.71 mmol, 1 eq) in dioxane (400 mL) and H₂O (80 mL) was added Na₂CO (34 g, 321.78 mmol, 2.5 eq) and Pd(dppf)Cl₂ (4.7 g, 6.44 mmol, 0.05 eq). Then the mixture was stirred at 90° C. for 2 hours under N₂ atmosphere. The reaction mixture was filtered. The filtrate was quenched with H₂O (600 mL) and extracted with ethyl acetate (2×600 mL). The organic phases were washed with brine (2×600 mL), dried over anhydrous Na₂SO₄, filtered and concentrated in vacuum. The residue was purified by silica gel column chromatography (SiO₂, petroleum ether:ethyl acetate, 1:0 to 1:1) to give the title compound (13 g, 43.7% yield) as a grey solid.

¹H NMR (400 MHz, CDCl₃) δ 8.49 (d, 2H), 7.33 (dd, 2H), 6.92 (dd, 1H), 6.73 (dd, 1H), 5.85 (s, 1H), 3.29-3.22 (m, 1H), 1.20 (d, 6H).

LCMS: m/z 230.1 (M−H)⁻ (ES⁻).

### Step B: 4-(2-((3-bromo-1-((2-(trimethylsilyl)ethoxy)methyl)-1H-1,2,4-triazol-5-yl)oxy)-5-fluoro-3-isopropylphenyl)pyridine

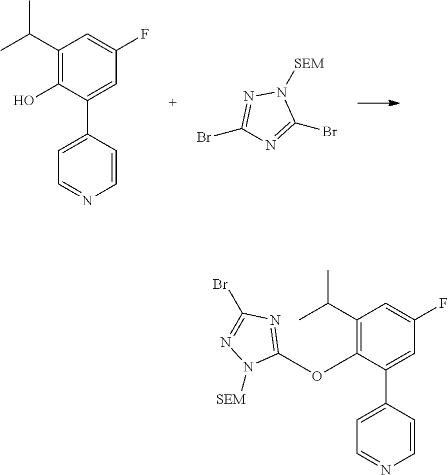 [SMILES: CC(C)c1cc(F)cc(-c2ccncc2)c1O ; C[Si](C)(C)CCOCn1nc(Br)nc1Br ; CC(C)c1cc(F)cc(-c2ccncc2)c1Oc1nc(Br)nn1COCC[Si](C)(C)C]

To a solution of 4-fluoro-2-isopropyl-6-(pyridin-4-yl)phenol (5 g, 21.62 mmol, 1 eq) in DMF (70 mL) was added NaH (1.30 g, 32.43 mmol, 60% purity in mineral oil, 1.5 eq) at 0° C. Then the mixture was stirred at 0° C. for 1 hour. To the above solution was added a solution of 3,5-dibromo-1-((2-(trimethylsilyl)ethoxy)methyl)-1H-1,2,4-triazole (Intermediate B6) (7.72 g, 10.81 mmol, 0.5 eq) in DMF (10 mL). Then the resulting mixture was stirred at 80° C. for 3 hours. The reaction mixture was quenched with H₂O (300 mL) and extracted with ethyl acetate (2×300 mL). The organic phases were washed with brine (2×500 mL), dried over anhydrous Na₂SO₄, filtered and concentrated in vacuum. The residue was purified by silica gel column chromatography (SiO₂, petroleum ether:ethyl acetate, 8:1 to 4:1) to give the title compound (8.8 g, 64.4% yield, 80% purity on LCMS) as a yellow solid.

¹H NMR (400 MHz, CDCl₃) δ 8.57 (d, 2H), 7.27 (d, 2H), 7.12 (dd, 1H), 6.91 (dd, 1H), 5.17 (s, 2H), 3.66-3.56 (m, 2H), 3.06-3.02 (m, 1H), 1.22 (d, 6H), 0.92-0.86 (m, 2H), 0.01 (s, 9H).

LCMS: m/z 509.3 (M+H)⁺ (ES⁺)

### Step C: methyl 3-((5-(4-fluoro-2-isopropyl-6-(pyridin-4-yl)phenoxy)-1-((2-(trimethylsilyl)ethoxy)methyl)-1H-1,2,4-triazol-3-yl)thio)propanoate

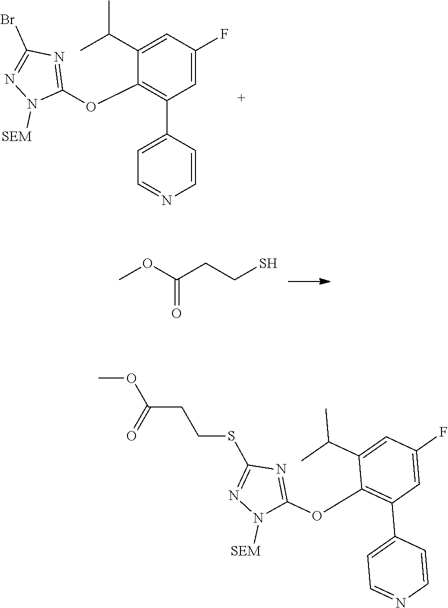

4-(2-((3-bromo-1-((2-(trimethylsilyl)ethoxy)methyl)-1H-1,2,4-triazol-5-yl)oxy)-5-fluoro-3-isopropylphenyl)pyridine (12 g, 23.71 mmol, 1 eq) was dissolved in dioxane (240 mL) and the resulting solution was bubbled with N₂. To the above solution was added methyl 3-mercaptopropanoate (5-78 g, 47.42 mmol, 2 eq), DIPEA (6.13 g, 47-42 mmol, 2 eq), Pd(dba)₂ (2.05 g, 3.56 mmol, 0.15 eq) and XantPhos (2.06 g, 3.56 mmol, 0.15 eq). The reaction mixture was degassed in vacuum and purged with N₂, and then heated to 100° C. and stirred for 16 hours. The reaction mixture was quenched with water (200 mL) and extracted with ethyl acetate (3×200 mL). The organic layers were dried over anhydrous Na₂SO₄, filtered and concentrated in vacuum. The residue was purified by silica gel column chromatography (SiO₂, petroleum ether:ethyl acetate, 10:1 to 1:1) to give the title compound (9 g, 66.7% yield, 96% purity on LCMS) as a yellow oil.

¹H NMR (400 MHz, CDCl₃) δ 8.58 (d, 2H), 7.30 (d, 2H), 7.11 (dd, 1H), 6.92 (dd, 1H), 5.16 (s, 2H), 3.69 (s, 3H), 3.59 (t, 2H), 3.15-3.09 (m, 3H), 2.65 (t, 2H), 1.25 (d, 6H), 0.90 (t, 2H), 0.02 (s, 9H).

LCMS: m/z 547.2 (M+H)⁺ (ES⁺)

### Step D: methyl 3-((5-(4-fluoro-2-isopropyl-6-(pyridin-4-yl)phenoxy)-1-((2-(trimethylsilyl)ethoxy)methyl)-1H-1,2,4-triazol-3-yl)sulfonyl)propanoate

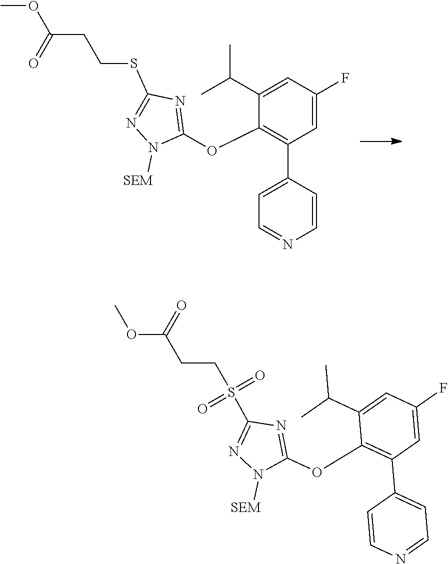 [SMILES: COC(=O)CCSc1nc(Oc2c(-c3ccncc3)cc(F)cc2C(C)C)n(COCC[Si](C)(C)C)n1 ; COC(=O)CCS(=O)(=O)c1nc(Oc2c(-c3ccncc3)cc(F)cc2C(C)C)n(COCC[Si](C)(C)C)n1]

To a solution of methyl 3-((5-(4-fluoro-2-isopropyl-6-(pyridin-4-yl)phenoxy)-1-((2-(trimethylsilyl)ethoxy)methyl)-1H-1,2,4-triazol-3-yl)thio)propanoate (1 g, 1.83 mmol, 1 eq) in DCM (20 mL) was added m-CPBA (929 mg, 4.58 mmol, 85 wt. % in H₂O, 2.5 eq). The mixture was stirred at 25° C. for 12 hours. Then 4,4,4′,4′,5,5,5′,5′-octamethyl-2,2′-bi(1,3,2-dioxaborolane) (2.32 g, 9.15 mmol, 5 eq) was added to the above mixture, and the mixture was stirred at 25° C. for another 12 hours. The reaction mixture was quenched with saturated aqueous Na₂SO₃ solution (20 mL) and extracted with DCM (3×20 mL). The organic layers were dried over anhydrous Na₂SO₄, filtered and concentrated in vacuum. The residue was purified by silica gel column chromatography (SiO₂, petroleum ether:ethyl acetate, 10:1 to 1:1) to give the title compound (850 mg, 77.5% yield, 96.5% purity on LCMS) as a white solid.

¹H NMR (400 MHz, CDCl₃) δ 8.59 (d, 2H), 7.31 (d, 2H), 7.14 (dd, 1H), 6.97 (dd, 1H), 5.35 (s, 2H), 3.72 (s, 3H), 3.62 (t, 2H), 3.43 (t, 2H), 3.09-3.06 (m, 1H), 2.67 (t, 2H), 1.25 (d, 6H), 0.94 (t, 2H), 0.03 (s, 9H).

LCMS: m/z 579.2 (M+H)⁺ (ES⁺)

### Step E: sodium 5-(4-fluoro-2-isopropyl-6-(pyridin-4-yl)phenoxy)-1-((2-(trimethylsilyl)ethoxy)methyl)-1H-1,2,4-triazole-3-sulfinate

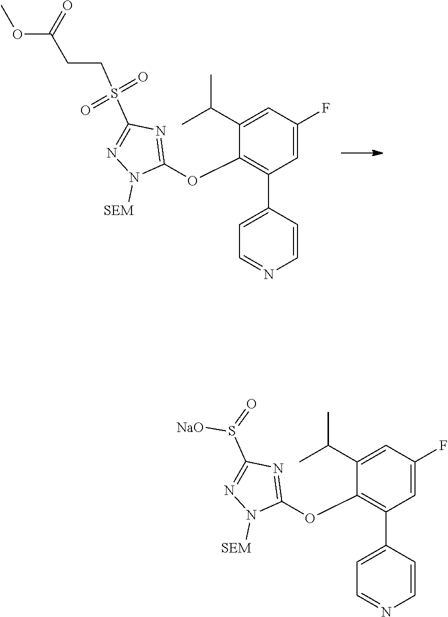

To a solution of methyl 3-((5-(4-fluoro-2-isopropyl-6-(pyridin-4-yl)phenoxy)-1-((2-(trimethylsilyl)ethoxy)methyl)-1H-1,2,4-triazol-3-yl)sulfonyl)propanoate (0.1 g, 172.95 μmol, 1 eq) in THF (3 mL) was added NaOMe (28 mg, 518.85 μmol, 3 eq) and MeOH (3 mL). The mixture was stirred at 25° C. for 5 hours. The reaction mixture was concentrated in vacuum to give the title compound (70 mg, crude) as a white solid, which was used directly in the next step.

LCMS: m/z 491.1 (M-Na)⁻ (ES⁻)

### Step F: 5-(4-fluoro-2-isopropyl-6-(pyridin-4-yl)phenoxy)-1-((2-(trimethylsilyl)-ethoxy)methyl)-1H-1,2,4-triazole-3-sulfonyl chloride

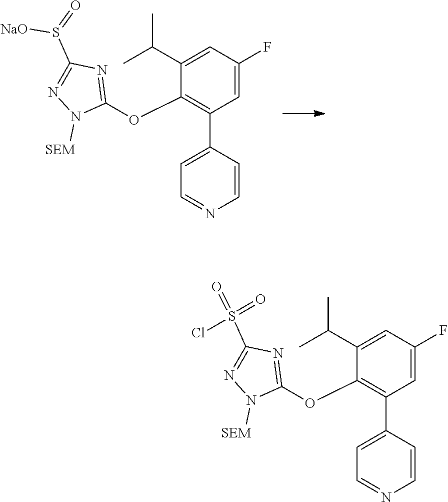

To a solution of sodium 5-(4-fluoro-2-isopropyl-6-(pyridin-4-yl)phenoxy)-1-((2-(trimethylsilyl)ethoxy)methyl)-1H-1,2,4-triazole-3-sulfinate (50 mg, 97.25 μmol, 1 eq) in AcOH (5 mL) and H₂O (2 mL) was added NCS (26 mg, 194.50 μmol, 2 eq) at 0° C. The mixture was stirred at 25° C. for 15 minutes. The reaction mixture was diluted with DCM (5 mL) and washed with water (3×5 mL) and brine (3×5 mL). The organic layer was dried over anhydrous Na₂SO₄ and filtered. The filtrate (theoretical amount: 51 mg) was used in next step directly.

### Intermediate B9: 5-((1,2,3,5,6,7-hexahydro-s-indacen-4-yl)amino)-1-((2-(trimethylsilyl)ethoxy)methyl)-1H-1,2,4-triazole-3-thiol

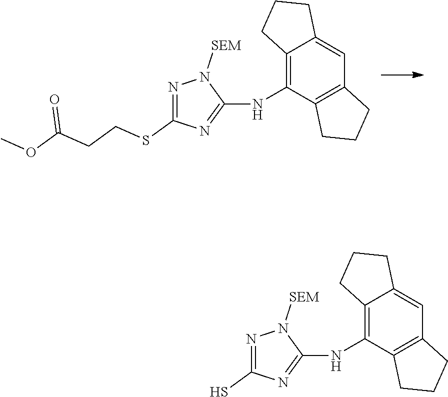 [SMILES: COC(=O)CCSc1nc(Nc2c3c(cc4c2CCC4)CCC3)n(COCC[Si](C)(C)C)n1 ; C[Si](C)(C)CCOCn1nc(S)nc1Nc1c2c(cc3c1CCC3)CCC2]

To a mixture of methyl 3-((5-((1,2,3,5,6,7-hexahydro-s-indacen-4-yl)amino)-1-((2-(trimethylsilyl)ethoxy)methyl)-1H-1,2,4-triazol-3-yl)thio)propanoate (Intermediate B², Step B) (6 g, 12.28 mmol, 1 eq) in THF (60 mL) and MeOH (60 mL) was added NaOMe (1.33 g, 24.55 mmol, 2 eq) in one portion at 0° C. Then the reaction mixture was stirred at 25° C. for 5 hours. The mixture was cooled to 0° C. and adjusted to pH=4 with 1 M aqueous HCl solution. The suspension was filtered, and the filter cake was dried in vacuum to give the title compound (3.8 g, 76.8% yield) as a yellow solid.

¹H NMR (400 MHz, DMSO-d₆) δ 12.37 (br s, 1H), 9.57 (br s, 1H), 7.05 (s, 1H), 5.20 (s, 2H), 3.64 (t, 2H), 2.84 (t, 4H), 2.65 (t, 4H), 2.03-1.98 (m, 4H), 0.91 (t, 2H), 0.03 (s, 9H).

LCMS: m/z 403.1 (M+H)⁺ (ES⁺).

### Intermediate B10: N-(1,2,3,5,6,7-hexahydro-s-indacen-4-yl)-3-((tetrahydro-2H-pyran-4-yl)sulfonyl)-1-((2-(trimethylsilyl)ethoxy)methyl)-1H-1,2,4-triazol-5-amine

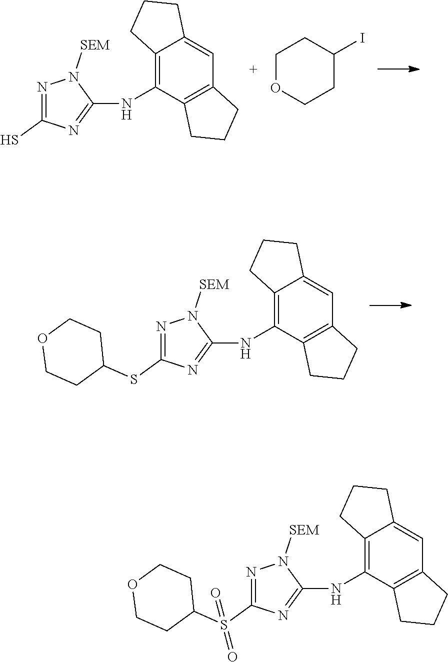

Step A: To a solution of 5-((1,2,3,5,6,7-hexahydro-s-indacen-4-yl)amino)-1-((2-(trimethylsilyl)ethoxy)methyl)-1H-1,2,4-triazole-3-thiol (Intermediate B9) (250 mg, 453.27 μmol, 1 eq) in DMF (4 mL) was added K₂CO₃ (75 mg, 543.93 μmol, 1.2 eq) and 4-iodotetrahydro-2H-pyran (96 mg, 453.27 μmol, 1 eq). The mixture was stirred at 25° C. for 2 hours. The reaction mixture was diluted with ethyl acetate (20 mL) and washed with saturated aqueous NH₄Cl solution (3×10 mL) and brine (3×10 mL). The organic layer was dried over anhydrous Na₂SO₄, filtered and concentrated in vacuum. The residue was purified by prep-TLC (SiO₂, petroleum ether:ethyl acetate, 2:1) to give N-(1,2,3,5,6,7-hexahydro-s-indacen-4-yl)-3-((tetrahydro-2H-pyran-4-yl)thio)-1-((2-(trimethylsilyl)ethoxy)methyl)-1H-1,2,4-triazol-5-amine (210 mg, 52.35% yield, 55% purity on LCMS) as a yellow oil.

¹H NMR (400 MHz, CDCl₃) δ 6.97 (s, 1H), 6.18 (s, 1H), 5.34 (s, 2H), 3.98-3.95 (m, 1H), 3.68-3.58 (m, 5H), 3.48 (t, 1H), 2.92-2.88 (m, 4H), 2.76-2.73 (m, 4H), 2.12-2.05 (m, 6H), 1.79-1.73 (m, 2H), 0.99-0.96 (m, 2H), 0.02 (s, 9H).

LCMS: m/z 487.3 (M+H)⁺ (ES⁺)

Step B: To a solution of N-(1,2,3,5,6,7-hexahydro-s-indacen-4-yl)-3-((tetrahydro-2H-pyran-4-yl)thio)-1-((2-(trimethylsilyl)ethoxy)methyl)-1H-1,2,4-triazol-5-amine (220 mg, 248.59 μmol, 1 eq) in DCM (4 mL) was added m-CPBA (101 mg, 497.18 μmol, 85 wt. % in H₂O, 2 eq). The mixture was stirred at 25° C. for 2 hours. The reaction mixture was quenched with saturated aqueous Na₂SO₃ solution (10 mL) and extracted with DCM (3×20 mL). The organic layers were dried over anhydrous Na₂SO₄ and filtered. To the filtrate was added 4,4,4′,4′,5,5,5′,5′-octamethyl-2,2′-bi(1,3,2-dioxaborolane) (250 mg), and the mixture was stirred for 12 hours at 25° C. Then the reaction mixture was concentrated in vacuum. The residue was purified by prep-TLC (SiO₂, petroleum ether:ethyl acetate, 2:1) to give the title compound (110 mg, 81.9% yield, 96% purity on LCMS) as a yellow solid.

¹H NMR (400 MHz, CDCl₃): δ 7.02 (s, 1H), 6.47 (s, 1H), 5.50 (s, 2H), 4.11-4.06 (m, 2H), 3.65 (t, 2H), 3.53-3.45 (m, 1H), 3.41-3.33 (m, 2H), 2.91 (t, 4H), 2.73 (t, 4H), 2.12-2.05 (m, 4H), 2.00-1.93 (m, 4H), 0.98 (t, 2H) and 0.03 (s, 9H).

LCMS: m/z 519.4 (M+H)⁺ (ES⁺).

### Intermediate B11: N-(1,2,3,5,6,7-hexahydro-s-indacen-4-yl)-3-((1-methyl-1H-pyrazol-4-yl)thio)-1-((2-(trimethylsilyl)ethoxy)methyl)-1H-1,2,4-triazol-5-amine

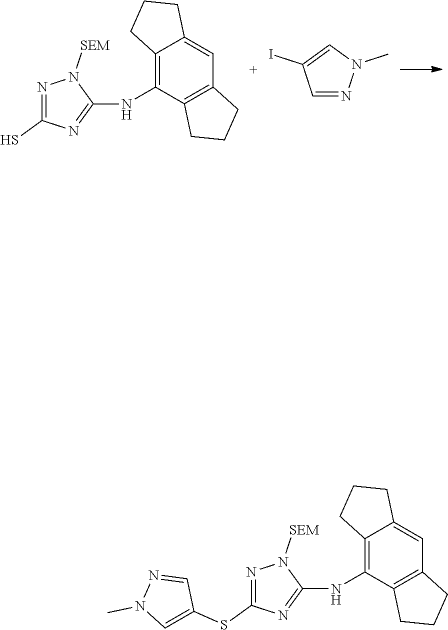

A mixture of 5-((1,2,3,5,6,7-hexahydro-s-indacen-4-yl)amino)-1-((2-(trimethylsilyl)-ethoxy)methyl)-1H-1,2,4-triazole-3-thiol (Intermediate B9) (300 mg, 521.57 μmol, 1 eq), 4-iodo-1-methyl-1H-pyrazole (108 mg, 521.57 μmol, 1 eq), CuI (198 mg, 1.04 mmol, 2 eq) and N,N′-dimethylethane-1,2-diamine (919 mg, 10.43 mmol, 20 eq) in dioxane (4 mL) was stirred at 70° C. under N₂ atmosphere for 2 hours. The reaction mixture was poured into water (10 mL) and extracted with ethyl acetate (3×10 mL). The organic layers were dried over anhydrous Na₂SO₄ filtered and concentrated in vacuum. The residue was purified by prep-TLC (SiO₂, petroleum ether:ethyl acetate, 1:1) to give the title compound (200 mg, 79.44% yield) as a yellow solid.

¹H NMR (400 MHz, CDCl₃) δ 7.62 (s, 1H), 7.58 (s, 1H), 6.95 (s, 1H), 6.18 (s, 1H), 5.28 (s, 2H), 3.90 (s, 3H), 3.59 (t, 2H), 2.88 (t, 4H), 2.67 (t, 4H), 2.06-2.03 (m, 4H), 0.96-0.92 (t, 2H), 0.02 (s, 9H).

LCMS: m/z 483.1 (M+H)⁺ (ES⁺).

### Intermediate B12: N-(1,2,3,5,6,7-hexahydro-s-indacen-4-yl)-3-((1-methyl-1H-pyrazol-4-yl)sulfonyl)-1-((2-(trimethylsilyl)ethoxy)methyl)-1H-1,2,4-triazol-5-amine

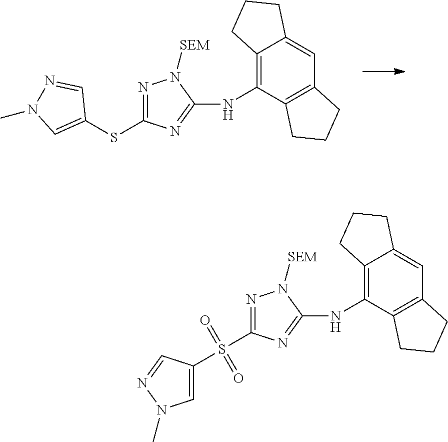 [SMILES: Cn1cc(Sc2nc(Nc3c4c(cc5c3CCC5)CCC4)n(COCC[Si](C)(C)C)n2)cn1 ; Cn1cc(S(=O)(=O)c2nc(Nc3c4c(cc5c3CCC5)CCC4)n(COCC[Si](C)(C)C)n2)cn1]

To a solution of N-(1,2,3,5,6,7-hexahydro-s-indacen-4-yl)-3-((1-methyl-1H-pyrazol-4-yl)thio)-1-((2-(trimethylsilyl)ethoxy)methyl)-1H-1,2,4-triazol-5-amine (Intermediate B¹¹) (200 mg, 414.32 μmol, 1 eq) in DCM (4 mL) was added m-CPBA (168 mg, 828.65 μmol, 85 wt. % in H₂O, 2 eq). The mixture was stirred at 25° C. for 2 hours. The reaction mixture was quenched with saturated aqueous Na₂SO₃ solution (10 mL) and extracted with DCM (3×20 mL). The organic layers were dried over anhydrous Na₂SO₄ and filtered. To the filtrate was added 4,4,4′,4′,5,5,5′,5′-octamethyl-2,2′-bi(1,3,2-dioxaborolane) (200 mg), and the mixture was stirred at 25° C. for 12 hours. The reaction mixture was concentrated in vacuum. The residue was purified by prep-TLC (SiO₂, petroleum ether:ethyl acetate, 1:1) to give the title compound (150 mg, 68.23% yield, 97% purity on LCMS) as a yellow solid.

¹H NMR (400 MHz, CDCl₃) δ 7.97 (s, 1H), 7.91 (s, 1H), 6.99 (s, 1H), 6.43 (s, 1H), 5.43 (s, 2H), 3.94 (s, 3H), 3.63 (t, 2H), 2.90 (t, 4H), 2.64 (t, 4H), 2.07-2.03 (m, 4H), 0.96 (t, 2H), 0.02 (s, 9H).

LCMS: m/z 515.2 (M+H)⁺ (ES⁺).

### Intermediate B13: N-(1,2,3,5,6,7-hexahydro-s-indacen-4-yl)-3-(pyridazin-3-ylthio)-1-((2-(trimethylsilyl)ethoxy)methyl)-1H-1,2,4-triazol-5-amine

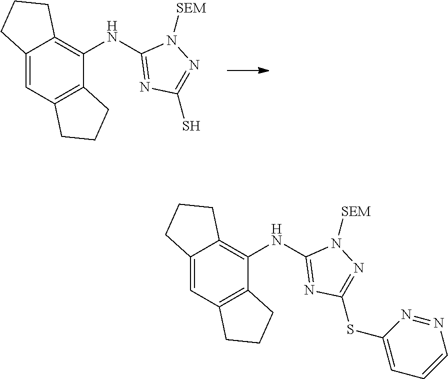 [SMILES: C[Si](C)(C)CCOCn1nc(S)nc1Nc1c2c(cc3c1CCC3)CCC2 ; C[Si](C)(C)CCOCn1nc(Sc2cccnn2)nc1Nc1c2c(cc3c1CCC3)CCC2]

A mixture of 3-bromopyridazine (98 mg, 620.92 μmol, 1 eq), 5-((1,2,3,5,6,7-hexahydro-s-indacen-4-yl)amino)-1-((2-(trimethylsilyl)ethoxy)methyl)-1H-1,2,4-triazole-3-thiol (Intermediate B9) (250 mg, 620.92 μmol, 1 eq), XantPhos (54 mg, 93.14 μmol, 0.15 eq), Pd(dba)₂ (54 mg, 93.14 μmol, 0.15 eq) and DIPEA (161 mg, 1.24 mmol, 2 eq) in dioxane (10 mL) was stirred at 100° C. for 2 hours under N₂. The reaction solution was diluted with water (20 mL) and extracted with EtOAc (3×15 mL). The organic layers were concentrated in vacuum, and the residue was purified by silica gel column chromatography (SiO₂, petroleum ether:ethyl acetate, 5:1 to 1:1) to give the title compound (230 mg, 73.9% yield, 96.2% purity on LCMS) as a brown gum.

¹H NMR (400 MHz, CDCl₃) δ 8.95 (dd, 1H), 7.49 (dd, 1H), 7.30-7.27 (m, 1H), 6.97 (s, 1H), 6.32 (s, 1H), 5.45 (s, 2H), 3.69-3.65 (m, 2H), 2.88 (t, 4H), 2.74 (t, 4H), 2.08-2.05 (m, 4H), 1.02-0.98 (m, 2H), 0.00 (s, 9H).

LCMS: m/z 481.3 (M+H)⁺ (ES⁺).

The following intermediates were synthesised following the general procedure for Intermediate B13:

| No. | Structure | ¹H NMR spectrum | LCMS |
|---|---|---|---|
| B14 | 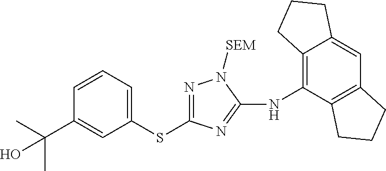 [SMILES: CC(C)(O)c1cccc(Sc2nc(Nc3c4c(cc5c3CCC5)CCC4)n(COCC[Si](C)(C)C)n2)c1] 2-(3-((5-((1,2,3,5,6,7-hexahydro-s- indacen-4-yl)amino)-1-((2- (trimethylsilyl)ethoxy)methyl)-1H-1,2,4- triazol-3-yl)thio)phenyl)propan-2-ol | ¹H NMR (400 MHz, CDCl₃) δ 7.62 (d, 1H), 7.36 (t, 2H), 7.27-7.24 (m, 1H), 6.94 (s, 1H), 6.21 (s, 1H), 5.37 (s, 2H), 3.64 (t, 2H), 2.86 (t, 4H), 2.68 (t, 4H), 2.05-2.00 (m, 4H), 1.54 (s, 6H), 0.97 (t, 2H), 0.03 (s, 9H). One exchangeable proton not observed. | m/z 537.2 (M + H)⁺ (ES⁺) |
| B15 | 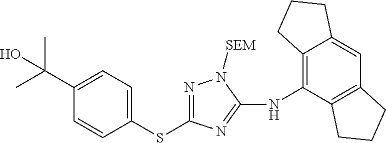 [SMILES: CC(C)(O)c1ccc(Sc2nc(Nc3c4c(cc5c3CCC5)CCC4)n(COCC[Si](C)(C)C)n2)cc1] 2-(4-((5-((1,2,3,5,6,7-hexahydro-s- indacen-4-yl)amino)-1-((2- (trimethylsilyl)ethoxy)methyl)-1H-1,2,4- triazol-3-yl)thio)phenyl)propan-2-ol | ¹H NMR (400 MHz, (CDCl₃) δ 7.47-7.40 (m, 4H), 6.94 (s, 1H), 6.20 (s, 1H), 5.37 (s, 2H), 3.63 (t, 2H), 2.86 (t, 4H), 2.68 (t, 4H), 2.03-2.01 (m, 4H), 1.56 (s, 6H), 0.96 (t, 2H), 0.02 (s, 9H). One exchangeable proton not observed. | m/z 537.3 (M + H)⁺ (ES⁺) |
| B16 | 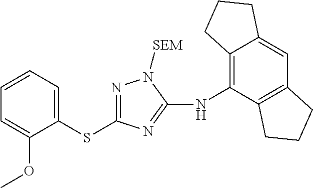 [SMILES: COc1ccccc1Sc1nc(Nc2c3c(cc4c2CCC4)CCC3)n(COCC[Si](C)(C)C)n1] N-(1,2,3,5,6,7-hexahydro-s-indacen-4- yl)-3-((2-methoxyphenyl)thio)-1-((2- (trimethylsilyl)ethoxy)methyl)-1H-1,2,4- triazol-5-amine | ¹H NMR (400 MHz, (CDCl₃) δ 7.31 (dd, 1H), 7.22-7.20 (m, 1H), 6.93 (s, 1H), 6.89-6.85 (m, 2H), 6.20 (s, 1H), 5.38 (s, 2H), 3.84 (s, 3H), 3.66- 3.61 (m, 2H), 2.86 (t, 4H), 2.70 (t, 4H), 2.07- 1.99 (m, 4H), 0.99-0.95 (m, 2H), 0.03 (s, 9H). | m/z 509.2 (M + H)⁺ (ES⁺) |
| B17 | 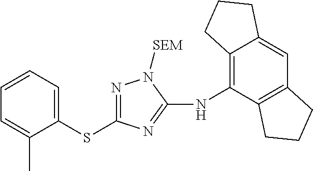 [SMILES: Cc1ccccc1Sc1nc(Nc2c3c(cc4c2CCC4)CCC3)n(COCC[Si](C)(C)C)n1] N-(1,2,3,5,6,7-hexahydro-s-indacen-4- yl)-3-(o-tolylthio)-1-((2-(trimethylsilyl)- ethoxy)methyl)-1H-1,2,4-triazol-5-amine | ¹H NMR (400 MHz, CDCl₃) δ 7.77-7.73 (m, 2H), 7.12-7.08 (m, 2H), 6.93 (s, 1H), 6.18 (s, 1H), 5.35 (s, 2H), 3.62 (t, 2H), 2.86 (t, 4H), 2.68 (t, 4H), 2.42 (s, 3H), 2.06-1.99 (m, 4H), 0.98-0.93 (m, 2H), 0.02 (s, 9H). | m/z 493.2 (M + H)⁺ (ES⁺) |
| B18 | 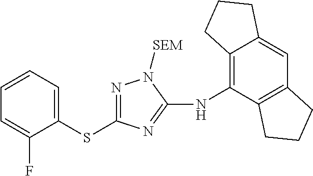 [SMILES: C[Si](C)(C)CCOCn1nc(Sc2ccccc2F)nc1Nc1c2c(cc3c1CCC3)CCC2] 3-((2-fluorophenyl)thio)-N-(1,2,3,5,6,7- hexahydro-s-indacen-4-yl)-1-((2- (trimethylsilyl)ethoxy)methyl)-1H-1,2,4- triazol-5-amine | ¹H NMR (400 MHz, CDCl₃) δ. 7.75 (d, 1H), 7.64-7.62 (m, 1H), 7.44- 7.42 (m, 1H), 7.09-7.07 (m, 1H), 6.93 (s, 1H), 6.20 (s, 1H), 5.36 (s, 2H), 3.64-3.59 (m, 2H), 2.86 (t, 4H), 2.67 (t, 4H), 2.06-1.98 (m, 4H), 0.97- 0.93 (m, 2H), 0.02 (s, 9H). | m/z 497.1 (M + H)⁺ (ES⁺) |
| B19 | 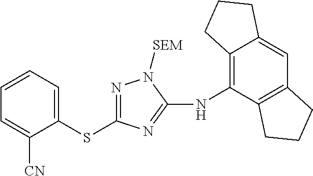 [SMILES: C[Si](C)(C)CCOCn1nc(Sc2ccccc2C#N)nc1Nc1c2c(cc3c1CCC3)CCC2] 2-((5-((1,2,3,5,6,7-hexahydro-s-indacen- 4-yl)amino)-1-((2-(trimethylsilyl)- ethoxy)methyl)-1H-1,2,4-triazol-3- yl)thio)benzonitrile | ¹H NMR (400 MHz, CDCl₃) δ 8.42 (dd, 1H), 7.53-7.48 (m, 1H), 7.28- 7.27 (m, 1H), 7.05-7.03 (m, 1H), 6.95 (s, 1H), 6.26 (s, 1H), 5.45 (s, 2H), 3.68-3.64 (m, 2H), 2.87 (t, 4H), 2.75 (t, 4H), 2.09-2.01 (m, 4H), 1.01- 0.97 (m, 2H), 0.03 (s, 9H). | m/z 504.2 (M + H)⁺ (ES⁺) |
| B20 | 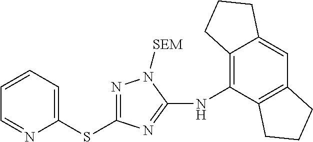 [SMILES: C[Si](C)(C)CCOCn1nc(Sc2ccccn2)nc1Nc1c2c(cc3c1CCC3)CCC2] N-(1,2,3,5,6,7-hexahydro-s-indacen-4- yl)-3-(pyridin-2-ylthio)-1-((2- (trimethylsilyl)ethoxy)methyl)-1H-1,2,4- triazol-5-amine | ¹H NMR (400 MHz, CDCl₃) δ 7.64 (dd, 1H), 7.51 (d, 1H), 7.47 (d, 1H), 7.34-7.30 (m, 1H), 6.95 (s, 1H), 6.26 (s, 1H), 5.40 (s, 2H), 3.67-3.63 (m, 2H), 2.87 (t, 4H), 2.70 (t, 4H), 2.07-2.00 (m, 4H), 1.00-0.95 (m, 2H), 0.032 (s, 9H). | m/z 480.3 (M + H)⁺ (ES⁺) |
| B21 | 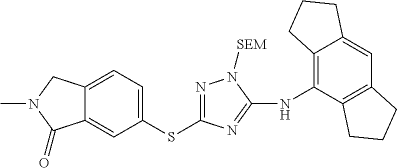 [SMILES: CN1Cc2ccc(Sc3nc(Nc4c5c(cc6c4CCC6)CCC5)n(COCC[Si](C)(C)C)n3)cc2C1=O] 6-((5-((1,2,3,5,6,7-hexahydro-s-indacen- 4-yl)amino)-1-((2-(trimethylsilyl)- ethoxy)methyl)-1H-1,2,4-triazol-3- yl)thio)-2-methylisoindolin-1-one | ¹H NMR (400 MHz, CDCl₃) δ 8.55 (s, 1H), 7.56-7.54 (m, 3H), 6.89 (s, 1H), 5.40 (s, 2H), 4.43 (s, 2H), 3.60 (t, 2H), 3.05 (s, 3H), 2.77 (t, 4H), 2.59 (t, 4H), 1.93-1.86 (m, 4H), 0.86 (t, 2H), 0.04 (s, 9H). | m/z 548.2 (M + H)⁺ (ES⁺) |
| B22 | 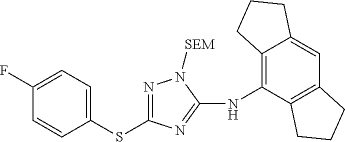 [SMILES: C[Si](C)(C)CCOCn1nc(Sc2ccc(F)cc2)nc1Nc1c2c(cc3c1CCC3)CCC2] 3-((4-fluorophenyl)thio)-N-(1,2,3,5,6,7- hexahydro-s-indacen-4-yl)-1-((2- (trimethylsilyl)ethoxy)methyl)-1H-1,2,4- triazol-5-amine | ¹H NMR (400 MHz, DMSO-d₆): δ 8.49 (s, 1H), 7.83-7.80 (m, 2H), 7.48- 7.44 (m, 2H), 6.92 (s, 1H), 5.37 (s, 2H), 3.58 (t, 2H), 2.78 (t, 4H), 2.57 (t, 4H), 1.95-1.87 (m, 4H), 0.89-0.84 (m, 2H), 0.04 (s, 9H). | m/z 497.3 (M + H)⁺ (ES⁺) |
| B23 | 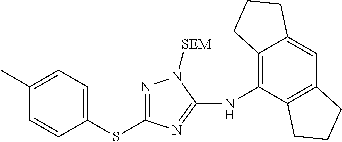 [SMILES: Cc1ccc(Sc2nc(Nc3c4c(cc5c3CCC5)CCC4)n(COCC[Si](C)(C)C)n2)cc1] N-(1,2,3,5,6,7-hexahydro-s-indacen-4- yl)-3-(p-tolylthio)-1-((2-(trimethylsilyl)- ethoxy)methyl)-1H-1,2,4-triazol-5-amine | ¹H NMR (400 MHz, DMSO-d₆), δ 8.44 (s, 1H), 7.25 (d, 2H), 7.12 (d, 2H), 6.90 (s, 1H), 5.37 (s, 2H), 3.58 (t, 2H), 2.78 (t, 4H), 2.57 (t, 4H), 2.26 (s, 3H), 1.94-1.87 (m, 4H), 0.86 (t, 2H) and 0.04 (s, 9H). | m/z 493.3 (M + H)⁺ (ES⁺) |
| B24 | 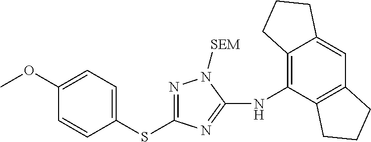 [SMILES: COc1ccc(Sc2nc(Nc3c4c(cc5c3CCC5)CCC4)n(COCC[Si](C)(C)C)n2)cc1] N-(1,2,3,5,6,7-hexahydro-s-indacen-4- yl)-3-((4-methoxyphenyl)thio)-1-((2- (trimethylsilyl)ethoxy)methyl)-1H-1,2,4- triazol-5-amine | ¹H NMR (400 MHz, DMSO-d₆) δ 8.41 (s, 1H), 7.80 (d, 1H), 7.46-7.45 (m, 1H), 7.36 (d, 1H), 7.35 (d, 1H), 6.89 (d, 1H), 5.34 (s, 2H), 3.74 (s, 3H), 3.56 (t, 2H), 2.78 (t, 4H), 2.56 (t, 4H), 1.94-1.87 (m, 4H), 0.85 (t, 2H), 0.05 (s, 9H). | m/z 509.4 (M + H)⁺ (ES⁺) |
| B25 | 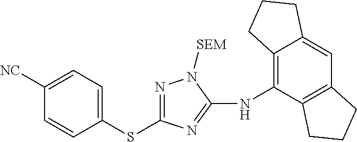 [SMILES: C[Si](C)(C)CCOCn1nc(Sc2ccc(C#N)cc2)nc1Nc1c2c(cc3c1CCC3)CCC2] 4-((5-((1,2,3,5,6,7-hexahydro-s-indacen- 4-yl)amino)-1-((2-(trimethylsilyl)- ethoxy)methyl)-1H-1,2,4-triazol-3- yl)thio)benzonitrile | ¹H NMR (400 MHz, DMSO-d₆): δ 8.63 (s, 1H), 7.79-7.74 (m, 2H), 7.48- 7.38 (m, 2H), 6.93 (s, 1H), 5.44 (s, 2H), 3.62 (t, 2H), 2.80 (t, 4H), 2.60 (t, 4H), 1.97-1.89 (m, 4H), 0.89 (t, 2H), 0.03 (s, 9H). | m/z 504.4 (M + H)⁺ (ES⁺) |
| B26 | 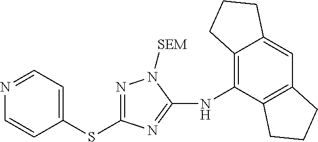 [SMILES: C[Si](C)(C)CCOCn1nc(Sc2ccncc2)nc1Nc1c2c(cc3c1CCC3)CCC2] N-(1,2,3,5,6,7-hexahydro-s-indacen-4- yl)-3-(pyridin-4-ylthio)-1-((2- (trimethylsilyl)ethoxy)methyl)-1H-1,2,4- triazol-5-amine | ¹H NMR (400 MHz, DMSO-d₆): δ 8.65 (s, 1H), 8.39 (d, 2H), 7.22 (d, 2H), 6.92 (s, 1H), 5.47 (s, 2H), 3.64 (t, 2H), 2.80 (t, 4H), 2.65 (t, 4H), 1.99- 1.92 (m, 4H), 0.90 (t, 2H), 0.02 (s, 9 H). | m/z 480.3 (M + H)⁺ (ES⁺) |
| B27 | 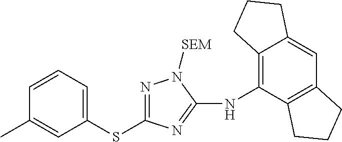 [SMILES: Cc1cccc(Sc2nc(Nc3c4c(cc5c3CCC5)CCC4)n(COCC[Si](C)(C)C)n2)c1] N-(1,2,3,5,6,7-hexahydro-s-indacen-4- yl)-3-(m-tolylthio)-1-((2-(trimethylsilyl)- ethoxy)methyl)-1H-1,2,4-triazol-5-amine | ¹H NMR (400 MHz, DMSO) δ 7.48-7.47 (m, 1H), 7.24-7.21 (m, 1H), 7.16 (s, 1H), 7.13-7.10 (m, 1H), 6.91 (s, 1H), 5.40 (s, 2H), 3.61 (t, 2H), 2.76 (t, 4H), 2.59 (t, 4H), 2.24 (s, 3H), 1.94-1.90 (m, 4H), 0.88 (t, 2H), -0.028 (s, 9 H). One exchangeable proton not observed. | m/z 493.2 (M + H)⁺ (ES⁺) |
| B28 | 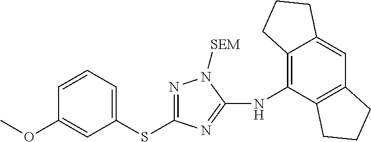 [SMILES: COc1cccc(Sc2nc(Nc3c4c(cc5c3CCC5)CCC4)n(COCC[Si](C)(C)C)n2)c1] N-(1,2,3,5,6,7-hexahydro-s-indacen-4- yl)-3-((3-methoxyphenyl)thio)-1-((2- (trimethylsilyl)ethoxy)methyl)-1H-1,2,4- triazol-5-amine | ¹H NMR (400 MHz, CDCl₃) δ 7.74-7.71 (m, 1H), 7.43-7.42 (m, 1H), 7.08-7.05 (m, 1H), 6.94 (s, 1H), 6.80-6.78 (m, 1H), 6.22 (s, 1H), 5.37 (s, 2H), 3.77 (s, 3H), 3.66- 3.61 (m, 2H), 2.87 (t, 4H), 2.69 (t, 4H), 2.07- 2.01 (m, 4H), 0.99-0.94 (m, 2H), 0.02 (s, 9H). | m/z 509.3 (M + H)⁺ (ES⁺) |
| B29 | 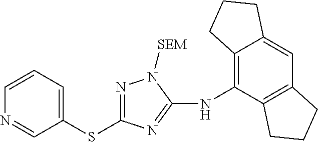 [SMILES: C[Si](C)(C)CCOCn1nc(Sc2cccnc2)nc1Nc1c2c(cc3c1CCC3)CCC2] N-(1,2,3,5,6,7-hexahydro-s-indacen-4- yl)-3-(pyridin-3-ylthio)-1-((2- (trimethylsilyl)ethoxy)methyl)-1H-1,2,4- triazol-5-amine | ¹H NMR (400 MHz, CDCl₃) δ 8.72 (d, 1H), 8.54 (d, 1H), 7.96-7.93 (m, 1H), 7.35 (dd, 1H), 6.97 (s, 1H), 6.42 (s, 1H), 5.36 (s, 2H), 3.62 (t, 2H), 2.87 (t, 4H), 2.68 (t, 4H), 2.09-2.01 (m, 4H), 0.97 (t, 2H), 0.02 (s, 9H). | m/z 480.3 (M + H)⁺ (ES⁺) |
| B30 | 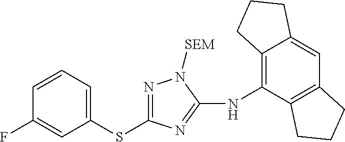 [SMILES: C[Si](C)(C)CCOCn1nc(Sc2cccc(F)c2)nc1Nc1c2c(cc3c1CCC3)CCC2] 3-((3-fluorophenyl)thio)-N-(1,2,3,5,6,7- hexahydro-s-indacen-4-yl)-1-((2- (trimethylsilyl)ethoxy)methyl)-1H-1,2,4- triazol-5-amine | ¹H NMR (400 MHz, CDCl₃) δ 7.64-7.62 (m, 2H), 7.44-7.42 (m, 2H), 6.95 (s, 1H), 6.25 (s, 1H), 5.40 (s, 2H), 3.67-3.62 (m, 2H), 2.87 (t, 4H), 2.70 (t, 4H), 2.08-2.02 (m, 4H), 0.98 (t, 2H), 0.03 (s, 9H). | m/z 497.3 (M + H)⁺ (ES⁺) |
| B31 | 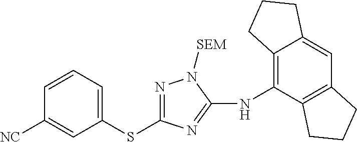 [SMILES: C[Si](C)(C)CCOCn1nc(Sc2cccc(C#N)c2)nc1Nc1c2c(cc3c1CCC3)CCC2] 3-((5-((1,2,3,5,6,7-hexahydro-s-indacen- 4-yl)amino)-1-((2-(trimethylsilyl)- ethoxy)methyl)-1H-1,2,4-triazol-3- yl)thio)benzonitrile | ¹H NMR (400 MHz, CDCl₃) δ 7.72 (t, 1H), 7.64-7.63 (m, 1H), 7.52- 7.49 (m, 1H), 7.42-7.39 (m, 1H), 6.97 (s, 1H), 6.28 (s, 1H), 5.41 (s, 2H), 3.68-3.63 (m, 2H), 2.88 (t, 4H), 2.71 (t, 4H), 2.10- 2.06 (m, 4H), 1.00-0.98 (m, 2H), 0.03 (s, 9H). | m/z 504.2 (M + H)⁺ (ES⁺) |
| B32 | 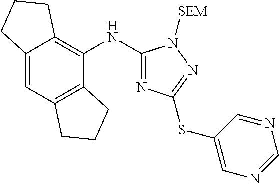 [SMILES: C[Si](C)(C)CCOCn1nc(Sc2cncnc2)nc1Nc1c2c(cc3c1CCC3)CCC2] N-(1,2,3,5,6,7-hexahydro-s-indacen-4- yl)-3-(pyrimidin-5-ylthio)-1-((2- (trimethylsilyl)ethoxy)methyl)-1H-1,2,4- triazol-5-amine | ¹H NMR (400 MHz, CDCl₃) δ 9.09 (s, 1H), 8.83 (s, 2H), 6.97 (s, 1H), 6.26 (s, 1H), 5.36 (s, 2H), 3.63 (t, 2H), 2.88 (t, 4H), 2.68 (t, 4H), 2.09-2.02 (m, 4H), 0.96 (t, 2H), 0.03 (s, 9H). | m/z 481.2 (M + H)⁺ (ES⁺) |
| B33 | 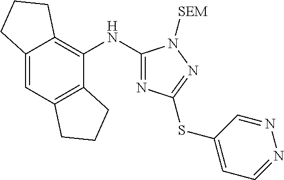 [SMILES: C[Si](C)(C)CCOCn1nc(Sc2ccnnc2)nc1Nc1c2c(cc3c1CCC3)CCC2] N-(1,2,3,5,6,7-hexahydro-s-indacen-4- yl)-3-(pyridazin-4-ylthio)-1-((2- (trimethylsilyl)ethoxy)methyl)-1H-1,2,4- triazol-5-amine | ¹H NMR (400 MHz, CDCl₃) δ 9.08 (t, 1H), 8.92 (d, 1H), 7.43 (dd, 1H), 6.99 (s, 1H), 6.36 (s, 1H), 5.45 (s, 2H), 3.70- 3.66 (m, 2H), 2.89 (t, 4H), 2.75 (t, 4H), 2.12- 2.05 (m, 4H), 1.03-0.98 (m, 2H), 0.04 (s, 9H). | m/z 481.1 (M + H)⁺ (ES⁺) |

### Intermediate B34:N-(1,2,3,5,6,7-hexahydro-s-indacen-4-yl)-3-((1-methyl-1H-pyrazol-3-yl)thio)-1-((2-(trimethylsilyl) ethoxy)methyl)-1H-1,2,4-triazol-5-amine

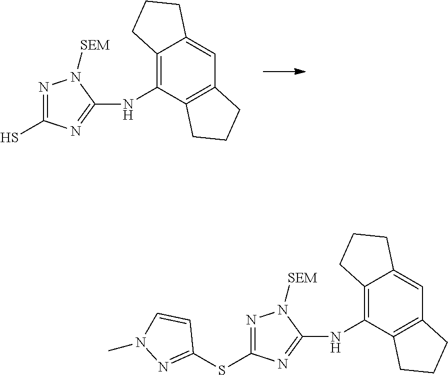 [SMILES: C[Si](C)(C)CCOCn1nc(S)nc1Nc1c2c(cc3c1CCC3)CCC2 ; Cn1ccc(Sc2nc(Nc3c4c(cc5c3CCC5)CCC4)n(COCC[Si](C)(C)C)n2)n1]

A mixture of 5-((1,2,3,5,6,7-hexahydro-s-indacen-4-yl)amino)-1-((2-(trimethylsilyl)-ethoxy)methyl)-1H-1,2,4-triazole-3-thiol (Intermediate B9)(230 ng, 417.01 μmol, 1 eq), 3-iodo-1-methyl-1H-pyrazole (86 mg, 417.01 μmol, 1 eq), Cu (158 mg, 834.02 μmol, 2 eq) and N,N′-dimethylethane-1,2-diamine (3 mg, 8.34 mmol, 20 eq) in dioxane (4 mL) was stirred at 70° C. under N₂ atmosphere for 2 hours. The reaction mixture was poured into water (10 mL) and extracted with EtOAc (3×10 mL). The organic layers were dried over anhydrous Na₂SO₄, filtered and concentrated in vacuum. The residue was purified by prep-TLC (SiO₂, petroleum ether:ethyl acetate, 1:1) to give the title compound (170 mg, 82.7% yield, 98% purity on LCMS) as a yellow solid.

¹H NMR (400 MHz, CDCl₃): δ 7.34 (d, 1H), 6.94 (s, 1H), 6.47 (d, 1H), 6.18 (s, 1H), 5.31 (s, 2H), 3.90 (s, 3H), 3.61 (t, 2H), 2.87 (t, 4H), 2.69 (t, 4H), 2.09-2.00 (m, 4H), 1.00-0.88 (m, 2H), 0.02 (s, 9H).

LCMS: m/z 483.2 (M+H)⁺ (ES⁺).

### Intermediate B35: 2-(3-((5-((1,2,3,5,6,7-hexahydro-s-indacen-4-yl)amino)-1-((2-(trimethylsilyl)ethoxy)methyl)-1H-1,2,4-triazol-3-yl)sulfonyl)phenyl)propan-2-ol

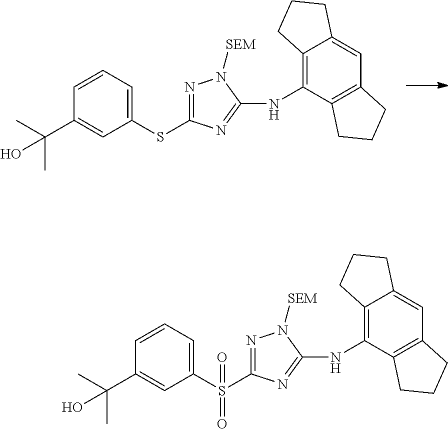 [SMILES: CC(C)(O)c1cccc(Sc2nc(Nc3c4c(cc5c3CCC5)CCC4)n(COCC[Si](C)(C)C)n2)c1 ; CC(C)(O)c1cccc(S(=O)(=O)c2nc(Nc3c4c(cc5c3CCC5)CCC4)n(COCC[Si](C)(C)C)n2)c1]

To a solution of 2-(3-((5-((1,2,3,5,6,7-hexahydro-s-indacen-4-yl)amino)-1-((2-(trimethylsilyl)ethoxy)methyl)-1H-1,2,4-triazol-3-yl)thio)phenyl)propan-2-ol (Intermediate B14) (150 mg, 279.43 μmol, 1 eq) in DCM (4 mL) was added m-CPBA (113 mg, 558.86 μmol, 85 wt. % in H₂O, 2 eq). The mixture was stirred at 25° C. for 2 hours. The reaction mixture was quenched with saturated aqueous Na₂SO₃ solution (10 mL), extracted with DCM (3×1 mL). The organic layers were dried over anhydrous Na₂SO₄ and filtered. To the filtrate was added 4,4,4′,4′,5,5,5′,5′-octamethyl-2,2′-bi(1,3,2-dioxaborolane (250 mg), and the solution was stirred for 12 hours. The mixture was concentrated in vacuum. The residue was purified by prep-TLC (SiO₂, petroleum ether:ethyl acetate 1:1) to give the title compound (60 mg, 37.7% yield) as a yellow oil. ¹H NMR (400 MHz, CDCl₃) δ 8.17 (t, 1H), 7.96 (m, 1H), 7.82-7.78 (m, 1H), 7.51 (t, 1H), 6.97 (s, 1H), 6.40 (s, 1H), 5.45 (s, 2H), 3.65-3.61 (m, 2H), 2.87 (t, 4H), 2.57 (t, 4H), 2.04-1.96 (m, 4H), 1.60 (s, 6H), 0.98-0.93 (m, 2H), 0.04 (s, 9H). One exchangeable proton not observed.

LCMS: m/z 569.2 (M+H)⁺ (ES⁺).

### Intermediate B36: 6-((5-((1,2,3,5,6,7-hexahydro-s-indacen-4-yl)amino)-1-((2-(trimethylsilyl)ethoxy)methyl)-1H-1,2,4-triazol-3-yl)sulfonyl)-2-methylisoindolin-1-one

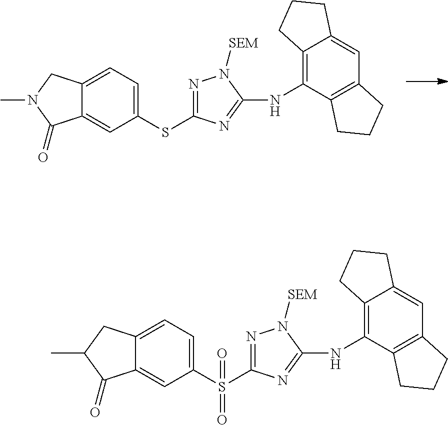 [SMILES: CN1Cc2ccc(Sc3nc(Nc4c5c(cc6c4CCC6)CCC5)n(COCC[Si](C)(C)C)n3)cc2C1=O ; CC1Cc2ccc(S(=O)(=O)c3nc(Nc4c5c(cc6c4CCC6)CCC5)n(COCC[Si](C)(C)C)n3)cc2C1=O]

A solution of ammonium molybdate (7 mg, 36.51 μmol, 0.25 eq) was dissolved in H₂O₂ (827 mg, 7.30 mmol, 30 wt. % in H₂O, 50 eq) at ° C. The above solution was added into a solution of 6-((5-((1,2,3,5,6,7-hexahydro-s-indacen-4-yl)amino)-1-((2-(trimethylsilyl)ethoxy)methyl)-1H-1,2,4-triazol-3-yl)thio)-2-methylisoindolin-1-one (Intermediate B21) (80 mg, 146.04 μmol, 1 eq) in EtOH (1 mL) at 0° C. The mixture was heated to 70° C. and stirred for 26 hours. The reaction mixture was filtered. The filter cake was washed with EtOH (3×10 mL), then added into a solution of 4,4,5,5-tetramethyl-2-(4,4,5,5-tetramethyl-1,3,2-dioxaborolan-2-yl)-1,3,2-dioxaborolane (185 mg, 5 eq) in DCM (2 mL). The mixture was stirred at 25° C. for 12 hours. The mixture was filtered and the filtrate was concentrated in vacuum to give the title compound (50 mg, crude) as a white solid.

¹H NMR (400 MHz, DMSO-d₆) δ 8.88 (s, 1H), 8.05-7.99 (m, 2H), 7.88-7.85 (m, 1H), 6.94 (s, 1H), 5.49 (s, 2H), 4.57 (s, 2H), 3.57-3.52 (m, 2H), 3.08 (s, 3H), 2.80-2.77 (m, 4H), 2.50-2.48 (m, 4H), 1.86-1.83 (m, 4H), 0.81-0.77 (m, 2H), −0.12 (s, 9H).

LCMS: m/z 480.2 (M+H)⁺ (ES⁺).

The following intermediates were synthesised following the general procedure for Intermediate B35, from the intermediate compounds indicated in the ‘From’ column.

| No. | Structure | ¹H NMR spectrum | LCMS | From |
|---|---|---|---|---|
| B37 | 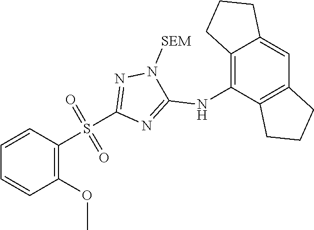 [SMILES: COc1ccccc1S(=O)(=O)c1nc(Nc2c3c(cc4c2CCC4)CCC3)n(COCC[Si](C)(C)C)n1] N-(1,2,3,5,6,7-hexahydro-s-indacen- 4-yl)-3-((2-methoxyphenyl)sulfonyl)- 1-((2-(trimethylsilyl)ethoxy)methyl)- 1H-1,2,4-triazol-5-amine | N/A | m/z 541.1 (M + H)⁺ (ES⁺) | B16 |
| B38 | 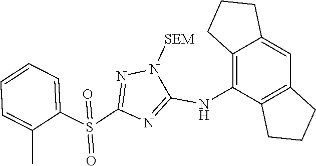 [SMILES: Cc1ccccc1S(=O)(=O)c1nc(Nc2c3c(cc4c2CCC4)CCC3)n(COCC[Si](C)(C)C)n1] N-(1,2,3,5,6,7-hexahydro-s-indacen- 4-yl)-3-(o-tolylsulfonyl)-1-((2- (trimethylsilyl)ethoxy)-methyl)-1H- 1,2,4-triazol-5-amine | ¹H NMR (400 MHz, CDCl₃) δ 8.18 (dd, 1H), 7.52-7.49 (m, 1H), 7.39-7.35 (m, 1H), 6.95 (s, 1H), 6.38 (s, 1H), 5.44 (s, 2H), 3.64-3.60 (m, 2H), 2.85 (t, 4H), 2.65 (s, 3H), 2.54 (t, 4H), 2.01-1.94 (m, 4H), 0.97-0.93 (m, 2H), 0.01 (s, 9 H). One exchangeable proton not observed. | m/z 525.3 (M + H)⁺ (ES⁺) | B17 |
| B39 | 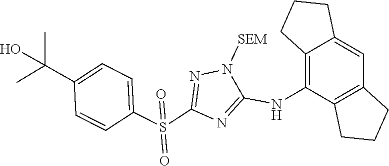 [SMILES: CC(C)(O)c1ccc(S(=O)(=O)c2nc(Nc3c4c(cc5c3CCC5)CCC4)n(COCC[Si](C)(C)C)n2)cc1] 2-(4-((5-((1,2,3,5,6,7-hexahydro-s- indacen-4-yl)amino)-1-((2- (trimethylsilyl)ethoxy)methyl)-1H- 1,2,4-triazol-3-yl)sulfonyl)phenyl)- propan-2-ol | ¹H NMR (400 MHz, CDCl₃) δ 8.05 (d, 2H), 7.67 (d, 2H), 6.98 (s, 1H), 6.39 (s, 1H), 5.43 (s, 2H), 3.66-3.59 (m, 2H), 2.88 (t, 4H), 2.57 (t, 4H), 2.04-1.97 (m, 4H), 1.60 (s, 6H), 0.97-0.93 (m, 2H), 0.01 (s, 9H). One exchangeable proton not observed. | m/z 569.2 (M + H)⁺ (ES⁺) | B15 |
| B40 | 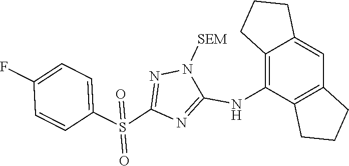 [SMILES: C[Si](C)(C)CCOCn1nc(S(=O)(=O)c2ccc(F)cc2)nc1Nc1c2c(cc3c1CCC3)CCC2] 3-((4-fluorophenyl)sulfonyl)-N- (1,2,3,5,6,7-hexahydro-s-indacen-4- yl)-1-((2-(trimethylsilyl)ethoxy)- methyl)-1H-1,2,4-triazol-5-amine | ¹H NMR (400 MHz, DMSO-d₆) δ 8.88 (s, 1H), 7.96-7.93 (m, 2H), 7.50 (t, 2H), 6.95 (s, 1H), 5.48 (s, 2H), 3.52 (t, 2H), 2.80 (t, 4H), 2.43 (t, 4H), 1.91-1.84 (m, 4H), 0.81 (t, 2H), 0.10 (s, 9H). | m/z 529.2 (M + H)⁺ (ES⁺) | B22 |
| B41 | 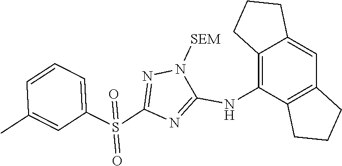 [SMILES: Cc1cccc(S(=O)(=O)c2nc(Nc3c4c(cc5c3CCC5)CCC4)n(COCC[Si](C)(C)C)n2)c1] N-(1,2,3,5,6,7-hexahydro-s-indacen- 4-yl)-3-(m-tolylsulfonyl)-1-((2- (trimethylsilyl)ethoxy)methyl)-1H- 1,2,4-triazol-5-amine | ¹H NMR (400 MHz, DMSO-d₆) δ 8.85 (s, 1H), 7.68-7.65 (m, 2H), 7.54-7.52 (m, 1H), 6.96 (s, 1H), 5.48 (s, 2H), 3.55 (t, 2H), 2.80 (t, 4H), 2.49 (t, 4H), 2.39 (s, 3H), 1.91-1.86 (m, 4H), 0.82 (t, 2H), −0.89 (s, 9H). One exchangeable proton not observed. | m/z 525.2 (M + H)⁺ (ES⁺) | B27 |
| B42 | 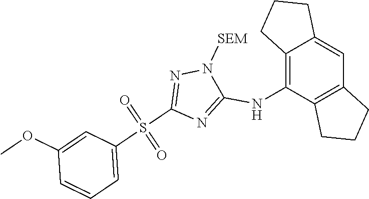 [SMILES: COc1cccc(S(=O)(=O)c2nc(Nc3c4c(cc5c3CCC5)CCC4)n(COCC[Si](C)(C)C)n2)c1] N-(1,2,3,5,6,7-hexahydro-s-indacen- 4-yl)-3-((3-methoxyphenyl)- sulfonyl)-1-((2-(trimethylsilyl)- ethoxy)methyl)-1H-1,2,4-triazol-5- amine | ¹H NMR (400 MHz, CDCl₃) δ 7.67 (d, 1H), 7.58 (t, 1H), 7.43 (t, 1H), 7.17 (dd, 1H), 6.98 (s, 1H), 6.34 (s, 1H), 5.44 (s, 2H), 3.84 (s, 3H), 3.62 (t, 2H), 2.88 (t, 4H), 2.56 (t, 4H), 2.05-1.98 (m, 4H), 0.95 (t, 2H), 0.01 (s, 9H). | m/z 541.3 (M + H)⁺ (ES⁺) | B28 |
| B43 | 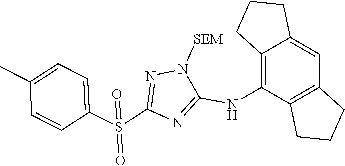 N-(1,2,3,5,6,7-hexahydro-s-indacen- 4-yl)-3-tosyl-1-((2-(trimethylsilyl) ethoxy)methyl)-1H-1,2,4-triazol-5 amine | ¹H NMR (400 MHz, DMSO-d₆) δ 8.44 (s, 1H), 7.33 (d, 2H), 7.13 (d, 2H), 6.90 (s, 1H), 5.37 (s, 2H), 3.58 (t, 2H), 2.78 (t, 4H), 2.58 (t, 4H), 2.27 (s, 3H), 1.93- 1.89 (m, 4H), 0.86 (t, 2H), −0.05 (s, 9H). | m/z 535.2 (M + H)⁺ (ES⁺) | B23 |
| B44 | 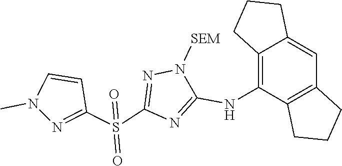 [SMILES: Cn1ccc(S(=O)(=O)c2nc(Nc3c4c(cc5c3CCC5)CCC4)n(COCC[Si](C)(C)C)n2)n1] N-(1,2,3,5,6,7-hexahydro-s-indacen- 4-yl)-3-((1-methyl-1H-pyrazol-3- yl)sulfonyl)-1-((2-(trimethylsilyl)- ethoxy)methyl)-1H-1,2,4-triazol-5- amine | ¹H NMR (400 MHz, CDCl₃) δ 7.44 (d, 1H), 6.98 (s, 1H), 6.91 (d, 1H), 6.44 (s, 1H), 5.44 (s, 2H), 3.99 (s, 3H), 3.66- 3.60 (m, 2H), 2.88 (t, 4H), 2.62 (t, 4H), 2.06-2.00 (m, 4H), 0.98-0.93 (m, 2H), 0.02 (s, 9H). | m/z 515.2 (M + H)⁺ (ES⁺) | B34 |
| B45 | 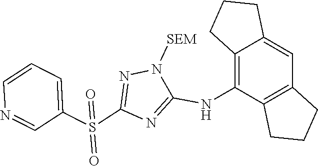 [SMILES: C[Si](C)(C)CCOCn1nc(S(=O)(=O)c2cccnc2)nc1Nc1c2c(cc3c1CCC3)CCC2] N-(1,2,3,5,6,7-hexahydro-s-indacen- 4-yl)-3-(pyridin-3-ylsulfonyl)-1-((2- (trimethylsilyl)ethoxy)methyl)-1H- 1,2,4-triazol-5-amine | ¹H NMR (400 MHz, CDCl₃) δ 9.25 (d, 1H), 8.85 (dd, 1H), 8.35 (dd, 1H), 7.50- 7.47 (m, 1H), 6.99 (s, 1H), 6.45 (s, 1H), 5.44 (s, 2H), 3.65- 3.60 (m, 2H), 2.89 (t, 4H), 2.57 (t, 4H), 2.05-2.00 (m, 4H), 0.97-0.93 (m, 2H), 0.03 (s, 9H). | m/z 512.2 (M + H)⁺ (ES⁺) | B29 |
| B46 | 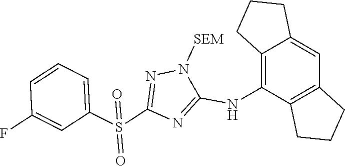 [SMILES: C[Si](C)(C)CCOCn1nc(S(=O)(=O)c2cccc(F)c2)nc1Nc1c2c(cc3c1CCC3)CCC2] 3-((3-fluorophenyl)sulfonyl)-N- (1,2,3,5,6,7-hexahydro-s-indacen-4- yl)-1-((2-(trimethylsilyl)ethoxy)- methyl)-1H-1,2,4-triazol-5-amine | ¹H NMR (400 MHz, CDCl₃) δ 7.89 (d, 1H), 7.79 (d, 1H), 7.54-7.51 (m, 1H), 7.34-7.33 (m, 1H), 6.99 (s, 1H), 6.43 (s, 1H), 5.44 (s, 2H), 3.64-3.59 (m, 2H), 2.88 (t, 4H), 2.70 (t, 4H), 2.05-1.99 (m, 4H), 0.97-0.92 (m, 2H), 0.00 (s, 9H). | m/z 529.2 (M + H)⁺ (ES⁺) | B30 |
| B47 | 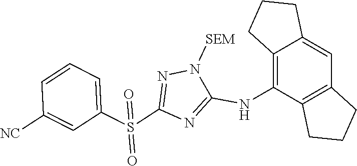 [SMILES: C[Si](C)(C)CCOCn1nc(S(=O)(=O)c2cccc(C#N)c2)nc1Nc1c2c(cc3c1CCC3)CCC2] 3-((5-((1,2,3,5,6,7-hexahydro-s- indacen-4-yl)amino)-1-((2- (trimethylsilyl)ethoxy)methyl)-1H- 1,2,4-triazol-3- yl)sulfonyl)benzonitrile | ¹H NMR (400 MHz, CDCl₃) δ 8.38 (t, 1H), 8.31 (dd, 1H), 7.91 (dd, 1H), 7.69 (t, 1H), 7.01 (s, 1H), 6.49 (s, 1H), 5.45 (s, 2H), 3.65-3.61 (m, 2H), 2.90 (t, 4H), 2.57 (t, 4H), 2.07- 1.99 (m, 4H), 0.98- 0.93 (m, 2H), 0.01 (s, 9H). | m/z 536.3 (M + H)⁺ (ES⁺) | B31 |
| B48 | 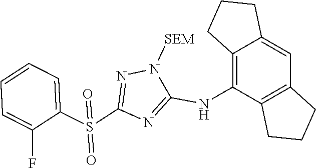 [SMILES: C[Si](C)(C)CCOCn1nc(S(=O)(=O)c2ccccc2F)nc1Nc1c2c(cc3c1CCC3)CCC2] 3-((2-fluorophenyl)sulfonyl)-N- (1,2,3,5,6,7-hexahydro-s-indacen-4- yl)-1-((2-(trimethylsilyl)ethoxy)- methyl)-1H-1,2,4-triazol-5-amine | ¹H NMR (400 MHz, CDCl₃) δ 8.14-8.10 (m, 1H), 7.67-7.62 (m, 1H), 7.36-7.32 (m, 1H), 7.19-7.15 (m, 1H), 6.95 (s, lH), 6.39 (s, 1H), 5.47 (s, 2H), 3.65-3.61 (m, 2H), 2.84 (t, 4H), 2.56 (t, 4H), 2.02- 1.94 (m, 4H), 0.98- 0.94 (m, 2H), 0.02 (s, 9H). | m/z 529.1 (M + H)⁺ (ES⁺) | B18 |
| B49 | 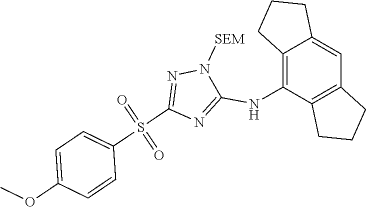 [SMILES: COc1ccc(S(=O)(=O)c2nc(Nc3c4c(cc5c3CCC5)CCC4)n(COCC[Si](C)(C)C)n2)cc1] N-(1,2,3,5,6,7-hexahydro-s-indacen- 4-yl)-3-((4-methoxyphenyl)- sulfonyl)-1-((2-(trimethylsilyl)- ethoxy)methyl)-1H-1,2,4-triazol-5- amine | ¹H NMR (400 MHz, DMSO-d₆) δ 8.81 (s, 1H), 7.79 (d, 2H), 7.14 (d, 2H), 6.95 (s, 1H), 5.47 (s, 2H), 3.85 (s, 3H), 3.52 (t, 2H), 2.80 (t, 4H), 2.44 (t, 4H), 1.89- 1.86 (m, 4H), 0.81 (t, 2H), 0.08 (s, 9H). | m/z 541.3 (M + H)⁺ (ES⁺) | B24 |
| B50 | 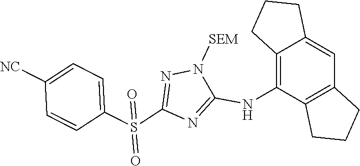 [SMILES: C[Si](C)(C)CCOCn1nc(S(=O)(=O)c2ccc(C#N)cc2)nc1Nc1c2c(cc3c1CCC3)CCC2] 4-((5-((1,2,3,5,6,7-hexahydro-s- indacen-4-yl)amino)-1-((2- (trimethylsilyl)ethoxy)methyl)-1H- 1,2,4-triazol-3- yl)sulfonyl)benzonitrile | ¹H NMR (400 MHz, DMSO-d₆) δ 8.94 (s, 1H), 8.15 (d, 2H), 8.04 (d, 2H), 6.96 (s, 1H), 5.49 (s, 2H), 3.52 (t, 2H), 2.80 (t, 4H), 2.42 (t, 4H), 1.89-1.84 (m, 4H), 0.81 (t, 2H), 0.00 (s, 9H). | m/z 536.2 (M + H)⁺ (ES⁺) | B25 |
| B51 | 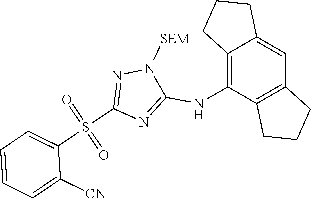 [SMILES: C[Si](C)(C)CCOCn1nc(S(=O)(=O)c2ccccc2C#N)nc1Nc1c2c(cc3c1CCC3)CCC2] 2-((5-((1,2,3,5,6,7-hexahydro-s- indacen-4-yl)amino)-1-((2- (trimethylsilyl)ethoxy)methyl)-1H- 1,2,4-triazol-3- yl)sulfonyl)benzonitrile | N/A | m/z 536.3 (M + H)⁺ (ES⁺) | B19 |

### Intermediate B52: 5-(2-isopropyl-6-(2-methoxypyridin-4-yl)phenoxy)-1-((2-(trimethylsilyl)ethoxy)methyl)-1H-1,2,4-triazole-3-sulfonyl chloride

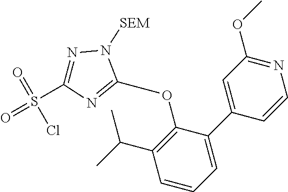 [SMILES: COc1cc(-c2cccc(C(C)C)c2Oc2nc(S(=O)(=O)Cl)nn2COCC[Si](C)(C)C)ccn1]

### Step A: 4-(2-((3-bromo-1-((2-(trimethylsilyl)ethoxy)methyl)-1H-1,2,4-triazol-5-yl)oxy)-3-isopropylphenyl)-2-methoxypyridine

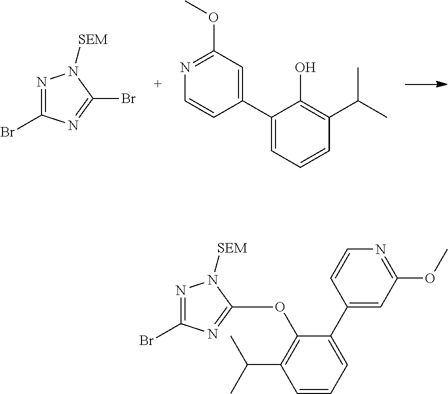 [SMILES: C[Si](C)(C)CCOCn1nc(Br)nc1Br ; COc1cc(-c2cccc(C(C)C)c2O)ccn1 ; COc1cc(-c2cccc(C(C)C)c2Oc2nc(Br)nn2COCC[Si](C)(C)C)ccn1]

To an ice-cooled solution of 2-isopropyl-6-(2-methoxypyridin-4-yl)phenol (Intermediate C33) (1.7 g, 6.99 mmol) in THF (40 mL) was added NaH (60% in mineral oil, 0.335 g, 8.38 mmol). The reaction was stirred for 10 min, then 3,5-dibromo-1-((2-(trimethylsilyl)ethoxy)methyl)-1H-1,2,4-triazole (2.495 g, 6.99 mmol) in THF (10 mL) was added. The reaction was then heated at 80° C. for 16 h, quenched with 1 M HCl (50 mL) and extracted with EtOAc (2×50 mL). The organic extract was dried and concentrated in vacuo. This residue was then dissolved in DMF (50 mL) and K₂CO₃ (0.966 g, 6.99 mmol) was added and the reaction was heated to 100° C. overnight, diluted with EtOAc (50 mL) and washed with water (2×50 mL) and brine (50 mL). The organic extract was dried (phase separator) and concentrated in vacuo. The product was purified by chromatography on silica gel (24 g column, 0-20% EtOAc/isohexane) and triturated with hexane (30 mL) to afford the title compound (1.5 g, 37%) as a flocculent white solid.

LCMS m/z 519.2/521.2 (M+H)⁺ (ES⁺).

¹H NMR (DMSO-d₆) δ 8.13 (d, J=5.3 Hz, 1H), 7.57 (dd, J=7.9, 1.7 Hz, 1H), 7.47 (t, J=7.7 Hz, 1H), 7.35 (dd, J=7.6, 1.6 Hz, 1H), 7.01 (dd, J=5.3, 1.5 Hz, 1H), 6.79 (t, J=0.9 Hz, 1H), 5.32 (s, 2H), 3.84 (s, 3H), 3.55 (t, J=8.0 Hz, 2H), 3.04 (h, J=7.1 Hz, 1H), 1.19 (d, J=6.9 Hz, 6H), 0.84 (t, J=8.0 Hz, 2H), −0.03 (s, 9H).

### Step B: methyl 3-((5-(2-isopropyl-6-(2-methoxypyridin-4-yl)phenoxy)-1-((2-(trimethylsilyl)ethoxy)methyl)-1H-1,24-triazol-3-yl)thio)propanoate

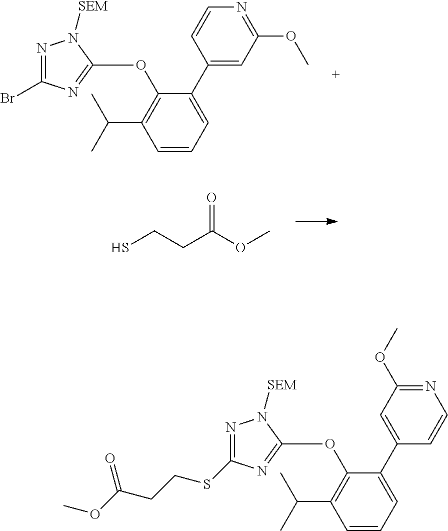

4-(2-((3-Bromo-1-((2-(trimethylsilyl)ethoxy)methyl)-1H-1,2,4-triazol-5-yl)oxy)-3-isopropylphenyl)-2-methoxypyridine (1.20 g, 2.310 mmol) was dissolved in dioxane (23 mL) and degassed with N₂. Methyl 3-mercaptopropanoate (0.512 mL, 4.62 mmol), XantPhos (0.200 g, 0.346 mmol) and Pd₂(dba)₃ (0.317 g, 0.346 mmol) were added followed by DIPEA (0.807 mL, 4.62 mmol). The reaction was degassed and heated to 100° C. overnight, diluted with EtOAc (40 mL), washed with water (30 mL) and 1 M HCl (3×30 mL). The organics were dried (phase separator), loaded onto silica and purified by chromatography on silica gel (24 g column, 0-50% MTBE/isohexane) to afford the title compound (1.14 g, 83%) as a yellow oil. LCMS m/z 559.3 (M+H)⁺ (ES⁺).

¹H NMR (DMSO-d₆) δ 8.12 (dd, J=5.3, 0.7 Hz, 1H), 7.55 (dd, J=7.8, 1.7 Hz, 1H), 7.45 (t, J=7.7 Hz, 1H), 7.34 (dd, J=7.6, 1.7 Hz, 1H), 7.00 (dd, J=5.3, 1.5 Hz, 1H), 6.78 (dd, J=1.5, 0.7 Hz, 1H), 5.28 (s, 2H), 3.83 (s, 3H), 3.59 (s, 3H), 3.56-3.51 (m, 2H), 3.07 (d, J=6.9 Hz, 1H), 3.03 (t, J=6.9 Hz, 2H), 2.59 (t, J=7.0 Hz, 2H), 1.21-1.14 (m, 6H), 0.85 (t, J=8.0 Hz, 2H), −0.03 (s, 9H).

### Step C: methyl 3-((5-(2-isopropyl-6-(2-methoxypyridin-4-yl)phenoxy)-1-((2-(trimethylsilyl)ethoxy)methyl)-1H-1,2,4-triazol-3-yl)sulfonyl)propanoate

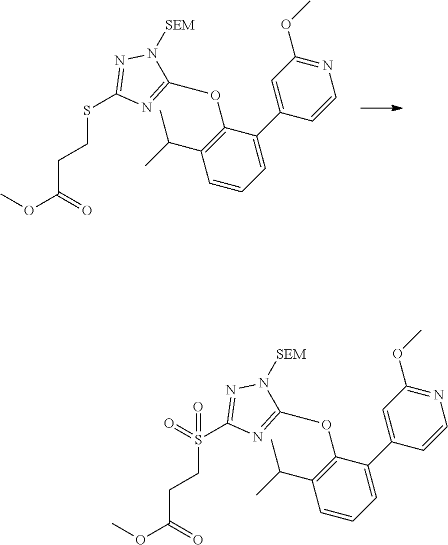 [SMILES: COC(=O)CCSc1nc(Oc2c(-c3ccnc(OC)c3)cccc2C(C)C)n(COCC[Si](C)(C)C)n1 ; COC(=O)CCS(=O)(=O)c1nc(Oc2c(-c3ccnc(OC)c3)cccc2C(C)C)n(COCC[Si](C)(C)C)n1]

m-CPBA (1.60 g, 7.14 mmol) was added to a solution of methyl 3-((5-(2-isopropyl-6-(2-methoxypyridin-4-yl)phenoxy)-1-((2-(trimethylsilyl)ethoxy)methyl)-1H-1,2,4-triazol-3-yl)thio)propanoate (1.14 g, 2.040 mmol) in DCM (50 mL) at 0° C. The reaction was stirred for 15 min and then warmed to RT, stirred for 5 h and quenched with aq. sodium sulfite. The aqueous layer was extracted with DCM (2×100 mL). The organic extracts were washed with sat aq NaHCO₃ (2×100 mL), dried (phase separator) and concentrated in vacuo. B₂Pin₂ (0.259 g, 1.020 mmol) and MeCN/MeOH (2:1, 30 mL) were added to half of the residue and the reaction was heated to 50° C. for 2 h, then cooled to RT. Ethane-1,2-diamine (0.102 mL, 1.530 mmol) was added and the reaction was stirred for 1 h at RT, diluted with water (30 mL) and extracted with MTBE (3×40 mL). The organic phases were dried (Na₂SO₄) and concentrated in vacuo to afford the title compound (566 mg, 40%) as a pale yellow solid.

¹H NMR (DMSO-d₆) δ 8.10 (d, J=5.3 Hz, 1H), 7.60 (dd, J=7.9, 1.7 Hz, 1H), 7.50 (t, J=7.7 Hz, 1H), 7.37 (dd, J=7.6, 1.7 Hz, 1H), 7.00 (dd, J=5.2, 1.5 Hz, 1H), 6.78 (s, 1H), 5.50 (s, 2H), 3.82 (s, 3H), 3.60 (s, 3H), 3.60-3.53 (m, 2H), 3.48 (t, J=7.2 Hz, 2H), 3.10 (p, J=6.9 Hz, 1H), 2.55-2.52 (m, 2H), 1.19 (d, J=6.9 Hz, 6H), 0.86 (t, J=8.0 Hz, 2H), −0.02 (s, 9H).

### Step D: Sodium 5-(2-isopropyl-6-(2-methoxypyridin-4-yl)phenoxy)-1-((2-(trimethylsilyl)ethoxy)methyl)-1H-1,2,4-triazole-3-sulfinate

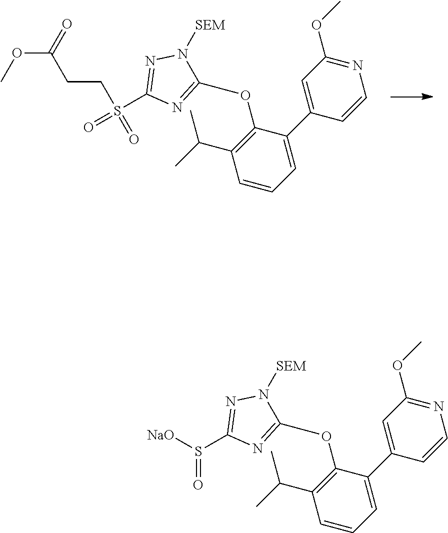

Prepared according to the general procedure of sodium 5-((1,2,3,5,6,7-hexahydro-s-indacen-4-yl)amino)-1-((2-(trimethylsilyl)ethoxy)methyl)-1H-1,2,4-triazole-3-sulfinate (Intermediate B2, Step D) from methyl 3-((5-(2-isopropyl-6-(2-methoxypyridin-4-yl)phenoxy)-1-((2-(trimethylsilyl)ethoxy)methyl)-1H-1,2,4-triazol-3-yl)sulfonyl)-propanoate to afford the title compound (134 mg, 95%) as a tan solid.

LCMS m/z 505.3 (M+H)⁺ (ES⁺).

### Step E: 5-(2-isopropyl-6-(2-methoxypyridin-4-yl)phenoxy)-1-((2-(trimethylsilyl)ethoxy)methyl)-1H-1,2,4-triazole-3-sulfonylchloride

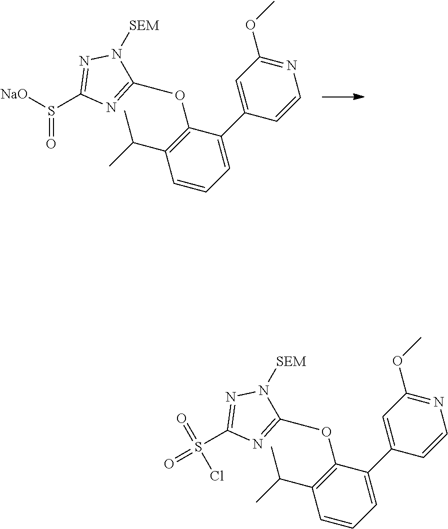

Sodium 5-(2-isopropyl-6-(2-methoxypyridin-4-yl)phenoxy)-1-((2-(trimethylsilyl)-ethoxy)methyl)-1H-1,2,4-triazole-3-sulfinate (134 mg, 0.254 mmol) was suspended in DCM (2 mL) at 0° C., then NCS (34 mg, 0.254 mmol) was added and the reaction was stirred for 1 h. The crude reaction mixture was then used directly in the next step without any purification.

### Intermediate B53: 5-((1,2,3,5,6,7-hexahydro-s-indacen-4-yl)oxy)-1-((2-(trimethylsilyl)ethoxy)methyl)-1H-1,2,4-triazole-3-sulfonyl chloride

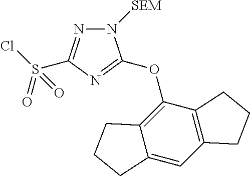 [SMILES: C[Si](C)(C)CCOCn1nc(S(=O)(=O)Cl)nc1Oc1c2c(cc3c1CCC3)CCC2]

### Step A: 3-bromo-5-((1,2,3,5,6,7-hexahydro-s-indacen-4-yl)oxy)-1-((2-(trimethylsilyl)ethoxy)methyl)-1H-1,2,4-triazole

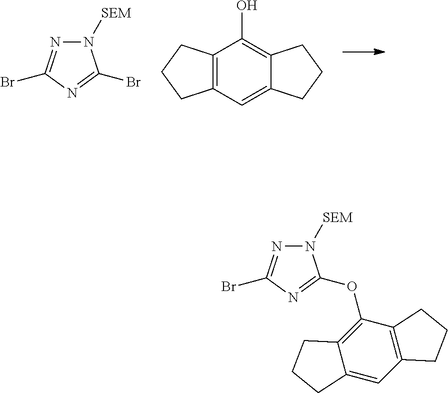

A solution of 3,5-dibromo-1-((2-(trimethylsilyl)ethoxy)methyl)-1H-1,2,4-triazole (0.60 g, 1.680 mmol), 1,2,3,5,6,7-hexahydro-s-indacen-4-ol(0.36 g, 1.860 mmol) and K₂CO₃ (0.93 g, 6.73 mmol) in DMF (3 mL) was heated to 100° C. for 16 h, cooled to RT and partitioned between water (20 mL) and MTBE (20 mL). The layers were separated and the organics were washed with brine (2×200 mL), dried (Na₂SO₄), loaded onto silica gel (15 g) and purified by chromatography on silica gel (40 g column, 0-50% EtOAc/isohexane) to afford the title compound (0.53 g, 51%) as a yellow solid.

LCMS m/z 450.2/452.2 (M+H)⁺ (ES⁺).

¹H NMR (DMSO-d₆) δ 7.06 (s, 1H), 5.42 (s, 2H), 3.68 (t, J=8.0 Hz, 2H), 2.86 (t, J=7.5 Hz, 4H), 2.65 (t, J=7.5 Hz, 4H), 2.02 (p, J=7.5 Hz, 4H), 0.90 (t, J=8.0 Hz, 2H), −0.02 (s, 9H).

### Step B: methyl 3-((5-((1,2,3,5,6,7-hexahydro-s-indacen-4-yl)oxy)-1-((2-(trimethylsilyl)ethoxy)methyl)-1H-1,2,4-triazol-3-yl)thio)propanoate

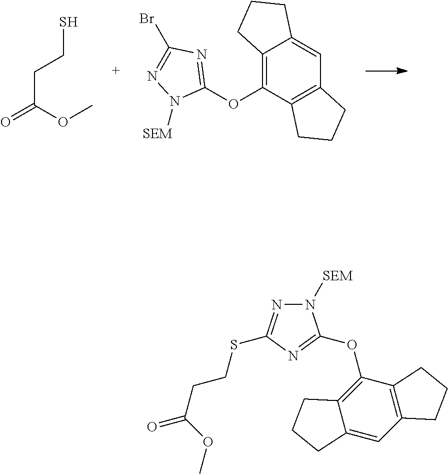

Prepared according to the general procedure of methyl 3-((5-(2-isopropyl-6-(2-methoxypyridin-4-yl)phenoxy)-1-((2-(trimethylsilyl)ethoxy)methyl)-1H-1,2,4-triazol-3-yl)thio)propanoate (Intermediate B52, Step B) from 3-bromo-5-((1,2,3,5,6,7-hexahydro-s-indacen-4-yl)oxy)-1-((2-(trimethylsilyl)ethoxy)methyl)-1H-1,2,4-triazole and methyl 3-mercaptopropanoate to afford the title compound (0.41 g, 88%) as a yellow oil.

LCMS m/z 490.3 (M+H)⁺ (ES⁺).

¹H NMR (DMSO-d₆) δ 7.05 (s, 1H), 5.39 (s, 2H), 3.72-3.64 (m, 2H), 3.62-3.59 (m, 3H), 3.16 (t, J=7.0 Hz, 2H), 2.86 (t, J=7.4 Hz, 4H), 2.74 (t, J=7.0 Hz, 2H), 2.65 (t, J=7.4 Hz, 4H), 2.01 (P, J=7.5 Hz, 4H), 0.97-0.85 (m 2H), −0.01 (s, 9H).

### Step C: methyl 3-((5-((1,2,3,5,6,7-hexahydro-s-indacen-4-yl)oxy)-1-((2-(trimethylsilyl)ethoxy)methyl)-1H-1,2,4-triazol-3-yl)sulfonyl)propanoate

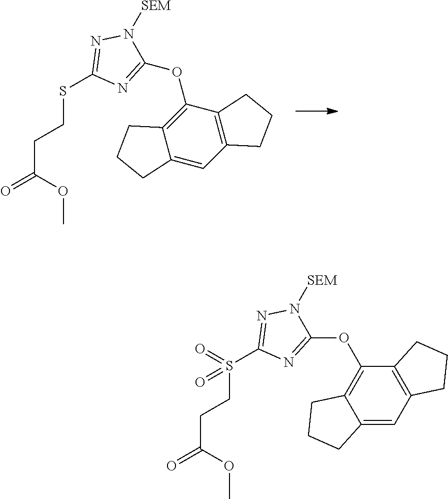 [SMILES: COC(=O)CCSc1nc(Oc2c3c(cc4c2CCC4)CCC3)n(COCC[Si](C)(C)C)n1 ; COC(=O)CCS(=O)(=O)c1nc(Oc2c3c(cc4c2CCC4)CCC3)n(COCC[Si](C)(C)C)n1]

Prepared according to the general procedure of methyl 3-((5-((1,2,3,5,6,7-hexahydro-s-indacen-4-yl)amino)-1-((2-(trimethylsilyl)ethoxy)methyl)-1H-1,2,4-triazol-3-yl)-sulfonyl)propanoate (Intermediate B2, Step C) from methyl 3-((5-((1,2,3,5,6,7-hexahydro-s-indacen-4-yl)oxy)-1-((2-(trimethylsilyl)ethoxy)methyl)-1H-1,2,4-triazol-3-yl)thio)propanoate and purified by chromatography on silica gel (24 g column, 0-30% EtOAc/isohexane) to afford the title compound (0.40 g, 83%) as a white solid.

LCMS m/z 522.5 (M+H)⁺ (ES⁺).

¹H NMR (DMSO-d₆) δ 7.09 (s, 1H), 5.58 (s, 2H), 3.73-3.65 (m, 2H), 3.62 (t, J=7.1 Hz, 2H), 3.58 (s, 3H), 2.87 (t, J=7.4 Hz, 4H), 2.73 (t, J=7.1 Hz, 2H), 2.67 (t, J=7.4 Hz, 4H), 2.02 (p, J=7.5 Hz, 4H), 0.95-0.86 (m, 2H), −0.02 (s, 9H).

### Step D: sodium 5-((1,2,3,5,6,7-hexahydro-s-indacen-4-yl)oxy)-1-((2-(trimethylsilyl)ethoxy)methyl)-1H-1,2,4-triazole-3-sulfinate

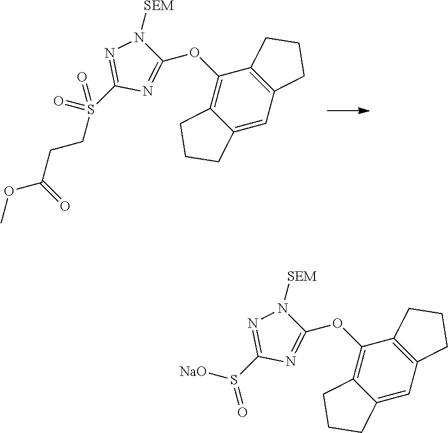

Prepared according to the general procedure of sodium 5-((1,2,3,5,6,7-hexahydro-s-indacen-4-yl)amino)-1-((2-(trimethylsilyl)ethoxy)methyl)-1H-1,2,4-triazole-3-sulfinate (Intermediate B2, Step D) from methyl 3-((5-((1,2,3,5,6,7-hexahydro-s-indacen-4-yl)oxy)-1-((2-(trimethylsilyl)ethoxy)methyl)-1H-1,2,4-triazol-3-yl)sulfonyl)propanoate to afford the title compound (0.30 g, 77%) as a white solid.

¹H NMR (methanol-d₄) δ 6.99 (s, 1H), 5.47 (s, 2H), 3.79-3.68 (m, 2H), 2.89 (t, J=7.5 Hz, 4H), 2.75 (t, J=7.4 Hz, 4H), 2.08 (p, J=7.3 Hz, 4H), 1.03-0.88 (m, 2H), 0.02 (s, 9H).

### Step E: 5-((1,2,3,5,6,7-hexahydro-s-indacen-4-yl)oxy)-1-((2-(trimethylsilyl)ethoxy)methyl)-1H-1,2,4-triazole-3-sulfonylchloride

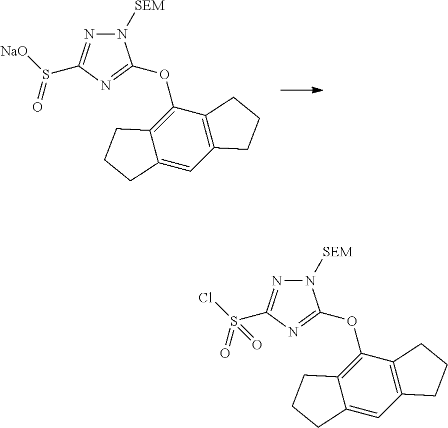

Sodium 5-((1,2,3,5,6,7-hexahydro-s-indacen-4-yl)oxy)-1-((2-(trimethylsilyl)ethoxy)-methyl)-1H-1,2,4-triazole-3-sulfinate (0.15 g, 0.33 mmol) was suspended in DCM (6 mL, 93 mmol) at 0° C. and NCS (0.042 g, 0.313 mmol) was added and the reaction mixture was stirred for 3 h at RT. The crude reaction mixture was then used directly in the preparation of examples without any purification assuming quantitative yield and 100% purity.

### Intermediate B4: 5-((5-(2-methoxypyridin-4-yl)-2,3-dihydro-1H-inden-4-yl)amino)-1-((2-(trimethylsilyl)ethoxy)methyl)-1H-1,2,4-triazole-3-thiol

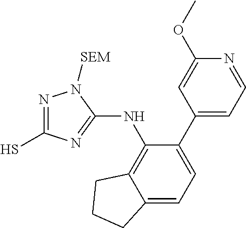 [SMILES: COc1cc(-c2ccc3c(c2Nc2nc(S)nn2COCC[Si](C)(C)C)CCC3)ccn1]

### Step A: 3-bromo-N-(5-(2-methoxypyridin-4-yl)-2,3-dihydro-1H-inden-4-yl)-1-((2-(trimethylsilyl)ethoxy)methyl)-1H-1,2,4-triazol-5-amine

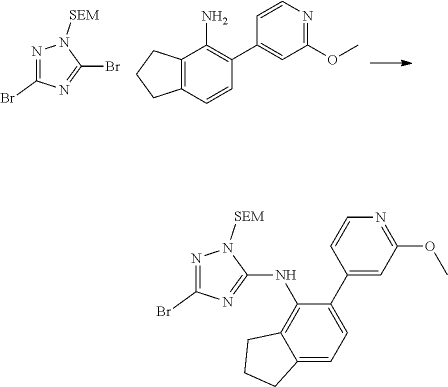

To an ice-cooled solution of 3,5-dibromo-1-((2-(trimethylsilyl)ethoxy)methyl)-1H-1,2,4-triazole (3.0 g, 8.40 mmol) and 5-(2-methoxypyridin-4-yl)-2,3-dihydro-1H-inden-4-amine (Intermediate R4) (2.23 g, 9.28 mmol) in THF (30 mL) under N₂ was added LiHMDS (1 M in THF, 16.8 mL, 16.8 mmol). The reaction was warmed to RT, stirred for 1 h, quenched with sat aq NH₄Cl (50 mL) and extracted with EtOAc (2×100 mL).

The organic extracts were dried (MgSO₄) and concentrated in vacuo. The product was dissolved in hot EtOAc (25 mL) and isohexane (200 mL) was added. The mixture was cooled to 0° C. and stirred for 15 min. The solid was collected to afford the title compound (2.19 g, 50%) as a tan solid.

LCMS m/z 516.3/518.3 (M+H)⁺ (ES⁺).

¹H NMR (DMSO-d₆) δ 8.79 (s, 1H), 8.11 (d, J=5.3 Hz, 1H), 7.27 (d, J=7.7 Hz, 1H), 7.20 (d, J=7.7 Hz, 1H), 6.91 (d, J=5.3 Hz, 1H), 6.74 (s, 1H), 5.26 (s, 2H), 3.83 (s, 3H), 3.53-3.47 (m, 2H), 2.96 (t, J=7.5 Hz, 2H), 2.67 (t, J=7.5 Hz, 2H), 2.02 (p, J=7.5 Hz, 2H), 0.82 (d, J=8.2 Hz, 2H), −0.03 (s, 9H).

### Step B: methyl 3-((5-((5-(2-methoxypyridin-4-yl)-2,3-dihydro-1H-inden-4-yl)amino)-1-((2-(trimethylsilyl)ethoxy)methyl)-1H-1,2,4-triazol-3-yl)thio)propanoate

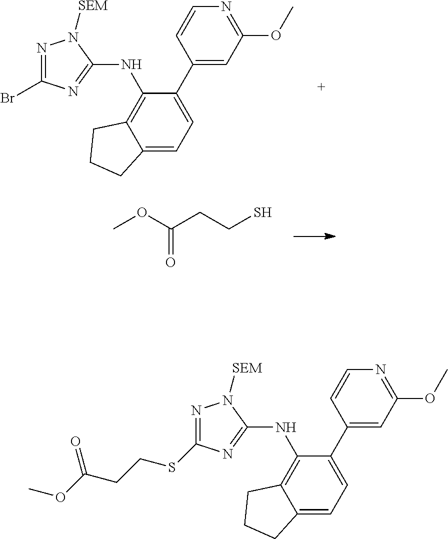

3-Bromo-N-(5-(2-methoxypyridin-4-yl)-2,3-dihydro-1H-inden-4-yl)-1-((2-(trimethylsilyl)ethoxy)methyl)-1H-1,2,4-triazol-5-amine (2.2 g, 4.26 mmol) was dissolved in 1,4-dioxane (25 mL) and degassed with N₂ for 10 min. Methyl 3-mercaptopropanoate (0.944 mL, 8.52 mmol), XantPhos (0.185 g, 0.319 mmol) and Pd₂(dba)₃ (0.293 g, 0.319 mmol) were added followed by DIPEA (1.49 mL, 8.52 mmol). The reaction mixture was degassed with N₂ and heated to 100° C. overnight, diluted with EtOAc (40 mL), washed with water (30 mL) and 1 M HCl (3×30 mL). The organic phases were dried (phase separator) and loaded onto silica. The crude product was purified by chromatography on silica gel (24 g column, 0-50% MTBE/isohexane) to afford the title compound (2.23 g, 76%) as a yellow oil.

LCMS m/z 556.0 (M+H)⁺ (ES⁺).

¹H NMR (DMSO-d₆) δ 8.52 (s, 1H), 8.09 (d, J=5.3 Hz, 1H), 7.23 (d, J=7.7 Hz, 1H), 7.17 (d, J=7.7 Hz, 1H), 6.91 (dd, J=5.3, 1.5 Hz, 1H), 6.75-6.73 (m, 1H), 5.24 (s, 2H), 3.82 (s, 3H), 3.60 (s, 3H), 3.53-3.47 (m, 2H), 3.08 (t, J=7.0 Hz, 2H), 2.94 (t, J=7.4 Hz, 2H), 2.70-2.61 (m, 4H), 2.04-1.96 (m, 2H), 0.86-0.73 (m, 2H), −0.04 (s, 9H).

### Step C: 5-((5-(2-methoxypyridin-4-yl)-2,3-dihydro-1H-inden-4-yl)amino)-1-((2-(trimethylsilyl)ethoxy)methyl)-1H-1,2,4-triazole-3-thiol

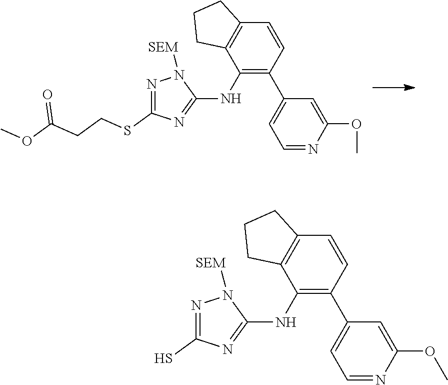

Methyl 3-((5-((5-(2-methoxypyridin-4-yl)-2,3-dihydro-1H-inden-4-yl)amino)-1-((2-(trimethylsilyl)ethoxy)methyl)-1H-1,2,4-triazol-3-yl)thio)propanoate (500 mg, 0.729 mmol) was dissolved in THF (7-5 mL) and MeOH (75 mL), cooled to 0° C. and sodium methoxide (0.270 mL, 1.46 mmol) was added. The reaction was warmed to RT and stirred for 90 min. The reaction mixture was cooled to 0° C., the pH was adjusted to 4 with acetic acid and the mixture was extracted with EtOAc (2×30 mL). The combined organic layers were dried (MgSO₄), filtered and concentrated in vacuo to give a yellow solid. This was triturated with isohexane (10 mL), filtered and dried under suction to give the title compound (0.25 g, 66%).

LCMS m/z 470.3 (M+H)⁺ (ES⁺); 468.2 (M−H)⁻ (ES⁻).

¹H NMR (DMSO-d₆) δ 12.56 (s, 1H), 9.70 (s, 1H), 8.16 (d, J=5.3 Hz, 1H), 7.35 (d, J=7.7 Hz, 1H), 7.22 (d, J=7.6 Hz, 1H), 6.94 (dd, J=5.3, 1.5 Hz, 1H), 6.75 (d, J=1.4 Hz, 1H), 5.12 (s, 2H), 3.86 (s, 3H), 3.55 (t, J=8.1 Hz, 2H), 2.98 (t, J=7.5 Hz, 2H), 2.17-1.84 (m, 4H), 0.90-0.81 (m, 2H), −0.01 (s, 9H).

### Intermediate B55: 5-((4-fluoro-2,6-diisopropylphenyl)amino)-1-((2-(trimethylsilyl)ethoxy)methyl)-1H-1,2,4-triazole-3-sulfonyl chloride

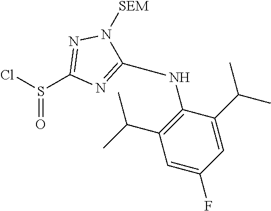 [SMILES: CC(C)c1cc(F)cc(C(C)C)c1Nc1nc(S(=O)Cl)nn1COCC[Si](C)(C)C]

### Step A: sodium 5-((4-fluoro-2,6-diisopropylphenyl)amino)-1-((2-(trimethylsilyl)ethoxy)methyl)-1H-1,2,4-triazole-3-sulfinate

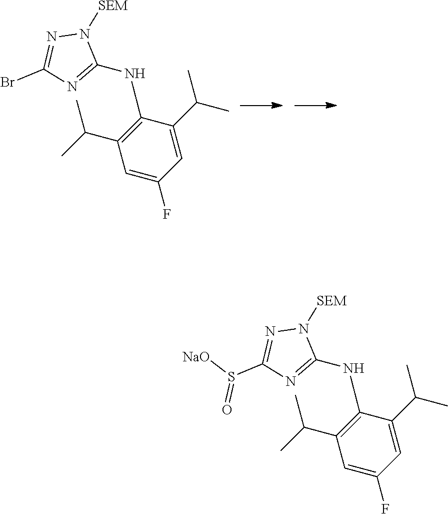

Prepared according to the general procedure of sodium 5-((1,2,3,5,6,7-hexahydro-s-indacen-4-yl)amino)-1-((2-(trimethylsilyl)ethoxy)methyl)-1H-1,2,4-triazole-3-sulfinate (Intermediate B2, Steps B to D) from 3-bromo-N-(4-fluoro-2,6-diisopropylphenyl)-1-((2-(trimethylsilyl)ethoxy)methyl)-1H-1,2,4-triazol-5-amine (Intermediate Bi) to afford the title compound (604 mg, 32% over three steps) as a light brown solid.

LCMS m/z 456.8 (M+H)⁺ (ES⁺); 454.9 (M−H)⁻ (ES⁻).

¹H NMR (DMSO-d₆) δ 8.00 (s, 1H), 6.96 (d, J=9.9 Hz, 2H), 5.37 (s, 2H), 3.59 (t, J=8.1 Hz, 2H), 3.10 (sept, J=6.8 Hz, 2H), 1.09 (d, J=6.8 Hz, 12H), 0.89 (t, J=8.2 Hz, 2H), 0.01 (s, 9H).

### Step B: 5-((4-fluoro-2,6-diisopropylphenyl)amino)-1-((2-(trimethylsilyl)ethoxy)methyl)-1H-1,2,4-triazole-3-sulfonyl chloride

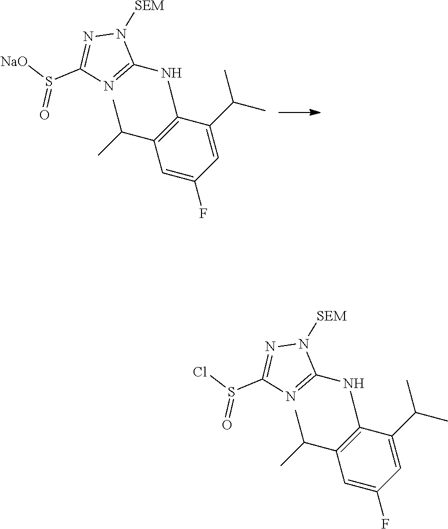

Prepared according to the general procedure of 5-(2-isopropyl-6-(2-methoxypyridin-4-yl)phenoxy)-1-((2-(trimethylsilyl)ethoxy)methyl)-1H-1,2,4-triazole-3-sulfonyl chloride (Intermediate B52, Step E) from sodium 5-((4-fluoro-2,6-diisopropylphenyl)-amino)-1-((2-(trimethylsilyl)ethoxy)methyl)-1H-1,2,4-triazole-3-sulfinate to afford the crude title compound which was used without work up or purification. An aliquot was quenched with morpholine and analysed by LCMS.

LCMS m/z 542.4 (M+H)⁺ (ES⁺); 540.2 (M−H)⁻ (ES⁻).

### Intermediate B56:5-((4-fluoro-2-isopropyl-6-(2-methoxypyridin-4-yl)phenyl)-amino)-1-((2-(trimethylsilyl)ethoxy)methyl)-1H-1,2,4-triazole-3-sulfonylchloride

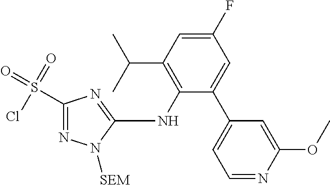 [SMILES: COc1cc(-c2cc(F)cc(C(C)C)c2Nc2nc(S(=O)(=O)Cl)nn2COCC[Si](C)(C)C)ccn1]

### Step A: 3-bromo-N-(4-fluoro-2-isopropyl-6-(2-methoxypyridin-4-yl)phenyl)-1-((2-(trimethylsilyl)ethoxy)methyl)-1H-1,2,4-triazol-5-amine

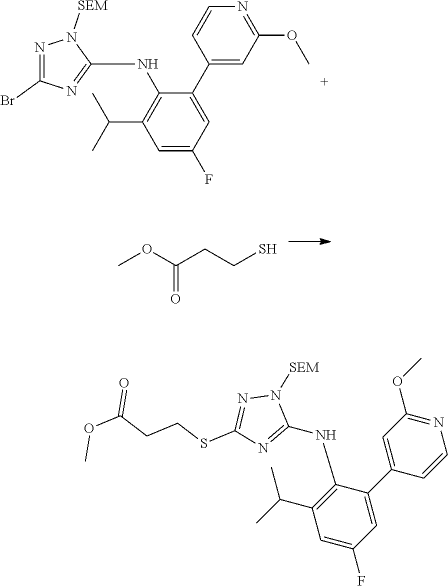

Prepared according to the general procedure of methyl 3-((5-(2-isopropyl-6-(2-methoxypyridin-4-yl)phenoxy)-1-((2-(trimethylsilyl)ethoxy)methyl)-1H-1,2,4-triazol-3-yl)thio)propanoate (Intermediate B52, Step B) from 3-bromo-N-(4-fluoro-2-isopropyl-6-(2-methoxypyridin-4-yl)phenyl)-1-((2-(trimethylsilyl)ethoxy)methyl)-1H-1,2,4-triazol-5-amine and methyl 3-mercaptopropanoate to afford the title compound (898 mg, 58%) as a pale yellow solid.

LCMS m/z 576.2 (M+H)⁺ (ES⁺).

¹H NMR (DMSO-d₆) δ 8.08 (d, J=5.3 Hz, 1H), 7.29 (dd, J=10.0, 3.0 Hz, 1H), 7.08 (dd, J=8.8, 3.0 Hz, 1H), 6.93 (dd, J=5.3, 1.5 Hz, 1H), 6.79-6.75 (m, 1H), 5.20 (s, 2H), 3.82 (s, 3H), 3.59 (s, 3H), 3.49-3.43 (m, 2H), 3.17-3.10 (m, 1H), 3.03 (t, J=7.0 Hz, 2H), 2.63 (t, J=7.0 Hz, 2H), 1.16-1.10 (m, 7H), 0.80 (dd, J=8.7, 7.4 Hz, 2H), −0.03 (s, 9H).

### Step B: methyl 3-((5-((4-fluoro-2-isopropyl-6-(2-methoxypyridin-4-yl)phenyl)amino-1-((2-(trimethylsilyl)ethoxy)methyl)-1H-1,2,4-triazol-3-yl)sulfonyl)propanoate

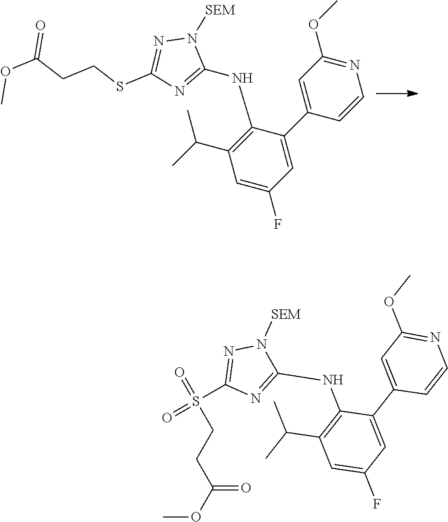 [SMILES: COC(=O)CCSc1nc(Nc2c(-c3ccnc(OC)c3)cc(F)cc2C(C)C)n(COCC[Si](C)(C)C)n1 ; COC(=O)CCS(=O)(=O)c1nc(Nc2c(-c3ccnc(OC)c3)cc(F)cc2C(C)C)n(COCC[Si](C)(C)C)n1]

Prepared according to the general procedure of methyl 3-((5-(2-isopropyl-6-(2-methoxypyridin-4-yl)phenoxy)-1-((2-(trimethylsilyl)ethoxy)methyl)-1H-1,2,4-triazol-3-yl)sulfonyl)propanoate (Intermediate B52, Step C) from methyl 3-((5-((4-fluoro-2-isopropyl-6-(2-methoxypyridin-4-yl)phenyl)amino)-1-((2-(trimethylsilyl)ethoxy)-methyl)-1H-1,2,4-triazol-3-yl)thio)propanoate to afford the title compound (695 mg, 59%) as a yellow oil.

LCMS m/z 608.3 (M+H)⁺ (ES⁺).

### Step C: sodium 5-((4-fluoro-2-isopropyl-6-(2-methoxypyridin-4-yl)phenyl)amino)-1-((2-(trimethylsilyl)ethoxy)methyl)-1H-1,2,4-triazole-3-sulfinate

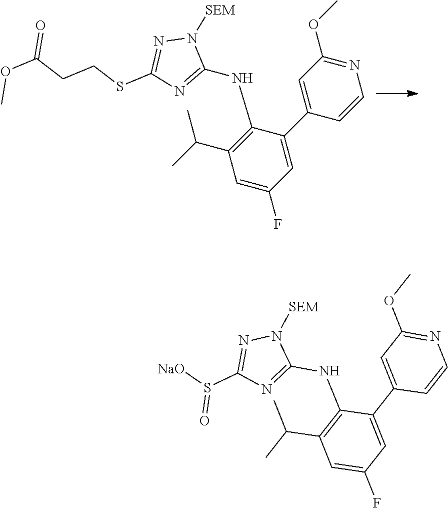

Prepared according to the general procedure of sodium 5-((1,2,3,5,6,7-hexahydro-s-indacen-4-yl)amino)-1-((2-(trimethylsilyl)ethoxy)methyl)-1H-1,2,4-triazole-3-sulfinate (Intermediate B2, Step D) from methyl 3-((5-((4-fluoro-2-isopropyl-6-(2-methoxy-pyridin-4-yl)phenyl)amino)-1-((2-(trimethylsilyl)ethoxy)methyl)-1H-1,2,4-triazol-3-yl)sulfonyl)propanoate to afford the title compound (610 mg, 88%) as a yellow solid.

LCMS m/z 522.3 (M+H)⁺ (ES⁺).

¹H NMR (DMSO-d₆) δ 8.06 (s, 1H), 7.28 (s, 1H), 7.07 (s, 1H), 6.96 (s, 1H), 6.82 (d, J=11.0 Hz, 1H), 5.19 (s, 2H), 3.78 (s, 3H), 3.52-3.43 (m, 2H)), 3.16-3.04 (m, 2H), 1.12 (d, J=6.8 Hz, 6H), 0.84-0.77 (m, 2H), −0.02 (d, J=7.5 Hz, 9H).

### Step D: 5-((4-fluoro-2-isopropyl-6-(2-methoxypyridin-4-yl)phenyl)amino)-1-((2-(trimethylsilyl)ethoxy)methyl)-1H-1,2,4-triazole-3-sulfonylchloride

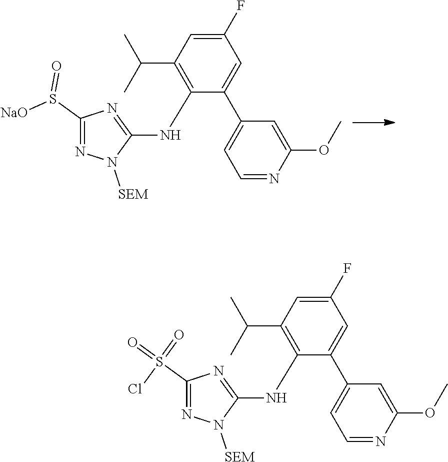

Prepared according to the general procedure of 5-(2-isopropyl-6-(2-methoxypyridin-4-yl)phenoxy)-1-((2-(trimethylsilyl)ethoxy)methyl)-1H-1,2,4-triazole-3-sulfonyl chloride (Intermediate B52, Step E) from sodium 5-((4-fluoro-2-isopropyl-6-(2-methoxy-pyridin-4-yl)phenyl)amino)-1-((2-(trimethylsilyl)ethoxy)methyl)-1H-1,2,4-triazole-3-sulfinate to afford the crude title compound which was used without work up or purification. An aliquot was quenched with morpholine and analysed by LCMS.

LCMS m/z 607.3 (M+H)⁺ (ES⁺).

### Intermediate B57: 5-(((2-(diethylamino)ethyl)sulfonyl)methyl)-N-(1,2,3,5,6,7-hexahydro-s-indacen-4-yl)-((2-(trimethylsilyl)ethoxy)methyl)-4H-1,2,4-triazol-3-amine

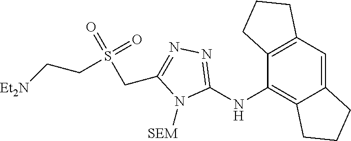

### Step A: methyl 5-bromo-((2-(trimethylsilyl)ethoxy)methyl)-4H-1,2,4-triazole-3-carboxylate

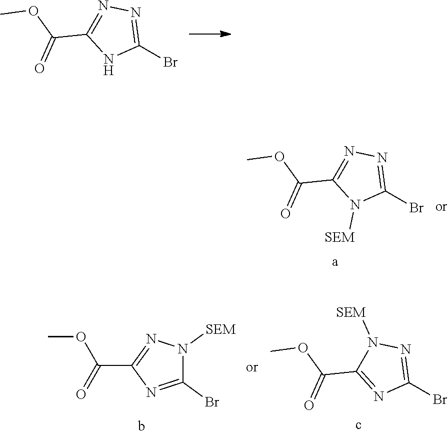

To a solution of methyl 5-bromo-4H-1,2,4-triazole-3-carboxylate (1.5 g, 7.28 mmol, 1 eq) in MeCN (20 mL) was added K₂CO₃ (1.51 g, 10.92 mmol, 1.5 eq) and SEM-Cl (1.34 g, 8.01 mmol, 1.1 eq). The mixture was stirred at 20° C. for 2 hours. The reaction solution was diluted with water (30 mL) and extracted with ethyl acetate (3×30 mL). The organic layers were concentrated in vacuo. The residue was purified by silica gel column chromatography (SiO₂, petroleum ether:ethyl acetate, 10:1 to 3:1) to give the title compound (1.2 g, 49.01% yield) as a colorless oil.

¹H NMR (400 MHz, CDCl₃) δ 5.86 (s, 2H), 4.02 (s, 3H), 3.67 (t, 2H), 0.93 (t, 2H), 0.01 (s, 9H). Single unidentified regioisomer obtained; location of SEM protecting group not determined.

LCMS: m/z 360.0 (M+Na)⁺ (ES⁺).

### Step B: methyl 5-((1,2,3,5,6,7-hexahydro-s-indacen-4-yl)amino)-((2-(trimethylsilyl)ethoxy)methyl)-4H-1,2,4-triazole-3-carboxylate

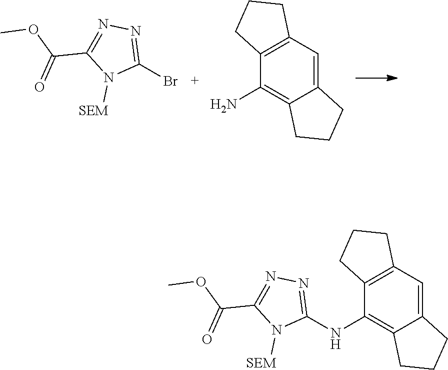

To a solution of methyl 5-bromo-((2-(trimethylsilyl)ethoxy)methyl)-4H-1,2,4-triazole-3-carboxylate (0.5 g, 1.49 mmol, 1 eq) and 1,2,3,5,6,7-hexahydro-s-indacen-4-amine (258 mg, 1.49 mmol, 1 eq) in dioxane (30 mL) was added Cs₂CO₃ (969 mg, 2.97 mmol, 2 eq), XantPhos (129 mg, 223.04 μmol, 0.15 eq) and Pd(dba)₂ (128 mg, 223.04 μmol, 0.15 eq) under N₂ atmosphere. Then the mixture was stirred at 100° C. for 2 hours. The reaction mixture was quenched with H₂O (30 mL) and extracted with ethyl acetate (2×30 mL). The organic phases were dried over anhydrous Na₂SO₄, filtered and concentrated in vacuum. The residue was purified by silica gel column chromatography (SiO₂, petroleum ether:ethyl acetate, 20:1) to give the title compound (0.6 g, 94.15% yield) as a yellow solid.

¹H NMR (400 MHz, CDCl₃) δ 8.31 (s, 1H), 6.92 (s, 1H), 5.64 (s, 2H), 3.92 (s, 3H), 3.65 (t, 2H), 2.86 (t, 4H), 2.71 (t, 4H), 2.01-1.98 (m, 4H), 0.89 (t, 2H), 0.00 (s, 9H). Single unidentified regioisomer obtained; location of SEM protecting group not determined.

LCMS: m/z 429.3 (M+H)⁺ (ES⁺).

### Step C: (5-((1,2,3,5,6,7-hexahydro-s-indacen-4-yl)amino)-((2-(trimethylsilyl)ethoxy)methyl)-4H-1,2,4-triazol-3-yl)methanol

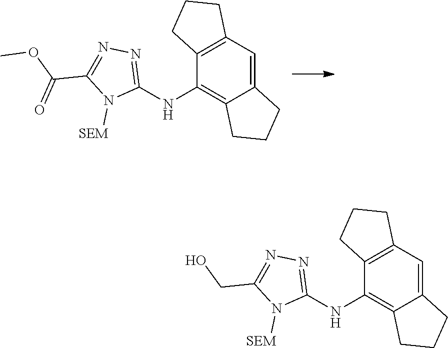 [SMILES: COC(=O)c1nnc(Nc2c3c(cc4c2CCC4)CCC3)n1COCC[Si](C)(C)C ; C[Si](C)(C)CCOCn1c(CO)nnc1Nc1c2c(cc3c1CCC3)CCC2]

To a solution of methyl 5-((1,2,3,5,6,7-hexahydro-s-indacen-4-yl)amino)-((2-(trimethylsilyl)ethoxy)methyl)-4H-1,2,4-triazole-3-carboxylate (620 mg, 1.45 mmol, 1 eq) in THF (40 mL) was added LiAlH₄ (110 mg, 2.89 mmol, 2 eq) at 0° C. Then the reaction mixture was stirred at 25° C. for 2 hours. The reaction mixture was quenched with H₂O (0.3 mL), dried over anhydrous Na₂SO₄, filtered and concentrated in vacuum. The residue was purified by silica gel column chromatography (SiO₂, petroleum ether:ethyl acetate, 4:1 to 3:1) to give the title compound (0.38 g, 65-58% yield) as a yellow solid.

¹H NMR (400 MHz, CDCl₃) δ 6.94 (s, 1H), 5.91 (s, 1H), 5.36 (s, 2H), 4.74 (s, 2H), 3.64 (t, 2H), 2.89 (t, 4H), 2.78 (t, 4H), 2.10-2.04 (m, 4H), 0.93 (t, 2H), 0.01 (s, 9H). One exchangeable proton not observed. Single unidentified regioisomer obtained; location of SEM protecting group not determined.

LCMS: m/z 401.3 (M+H)⁺ (ES⁺).

### Step D: 5-(chloromethyl)-N-(1,2,3,5,6,7-hexahydro-s-indacen-4-yl)-((2-(trimethylsilyl)ethoxy)methyl)-4H-1,2,4-triazol-3-amine

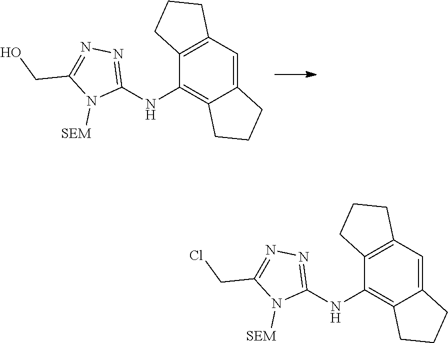 [SMILES: C[Si](C)(C)CCOCn1c(CO)nnc1Nc1c2c(cc3c1CCC3)CCC2 ; C[Si](C)(C)CCOCn1c(CCl)nnc1Nc1c2c(cc3c1CCC3)CCC2]

To a solution of (5-((1,2,3,5,6,7-hexahydro-s-indacen-4-yl)amino)-((2-(trimethylsilyl)-ethoxy)methyl)-4H-1,2,4-triazol-3-yl)methanol (0.35 g, 873.71 μmol, 1 eq) in CCl₄ (30 mL) was added PPh₃ (275 mg, 1.05 mmol, 1.2 eq). The mixture was stirred at 80° C. for 12 hours under N₂ atmosphere. The reaction mixture was concentrated in vacuum. The residue was purified by silica gel column chromatography (SiO₂, petroleum ether:ethyl acetate, 15:1 to 12:1) to give the title compound (0.19 g, 51.9% yield) as a yellow oil.

¹H NMR (400 MHz, CDCl₃) δ 6.94 (s, 1H), 5.39 (s, 2H), 4.64 (s, 2H), 3.64 (t, 2H), 2.89 (t, 4H), 2.78 (t, 4H), 2.10-2.04 (m, 4H), 0.94 (t, 2H), 0.01 (s, 9H). One exchangeable proton not observed. Single unidentified regioisomer obtained; location of SEM protecting group not determined.

LCMS: m/z 419.3 (M+1)+(ES⁺).

### Step E: 5-(((2-(diethylamino)ethyl)thio)methyl)-N-(1,2,3,5,6,7-hexahydro-s-indacen-4-yl)-((2-(trimethylsilyl)ethoxy)methyl)-4H-1,2,4-triazol-3-amine

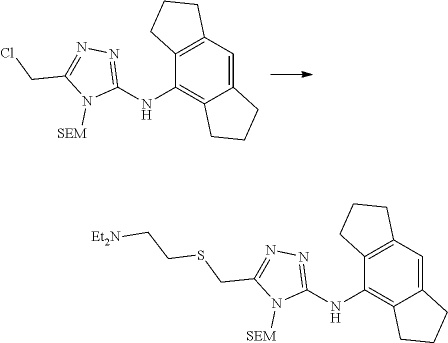

To a solution of 5-(chloromethyl)-N-(1,2,3,5,6,7-hexahydro-s-indacen-4-yl)-((2-(trimethylsilyl)ethoxy)methyl)-4H-1,2,4-triazol-3-amine (180 mg, 429.56 μmol, 1 eq) and 2-(diethylamino)ethanethiol (74 mg, 558.43 μmol, 1.3 eq) in EtOH (5 mL) was added KOH (48 mg, 859.12 μmol, 2 eq). Then the reaction mixture was stirred at 80° C. for 0.5 hour. The reaction mixture was quenched with H₂O (10 mL) and extracted with ethyl acetate (2×10 mL). The organic phases were dried over anhydrous Na₂SO₄, filtered and concentrated in vacuum. The residue was purified by prep-TLC (SiO₂, dichloromethane:methanol, 5:1) to give the title compound (0.12 g, 54.16% yield) as a yellow oil.

¹H NMR (400 MHz, CDCl₃) δ 6.93 (s, 1H), 5.78 (s, 1H), 5.38 (s, 2H), 3.83 (s, 2H), 3.63 (t, 2H), 2.89 (t, 4H), 2.81-2.74 (m, 8H), 2.71-2.51 (m, 4H), 2.10-2.04 (m, 4H), 1.12-1.00 (m, 6H), 0.93 (t, 2H), 0.02 (s, 9H). Single unidentified regioisomer obtained; location of SEM protecting group not determined.

LCMS: m/z 516.5 (M+1)+(ES⁺).

### Step F: 5-(((2-(diethylamino)ethyl)sulfonyl)methyl)-N-(1,2,3,5,6,7-hexahydro-s-indacen-4-yl)-((2-(trimethylsilyl)ethoxy)methyl)-4H-1,2,4-triazol-3-amine

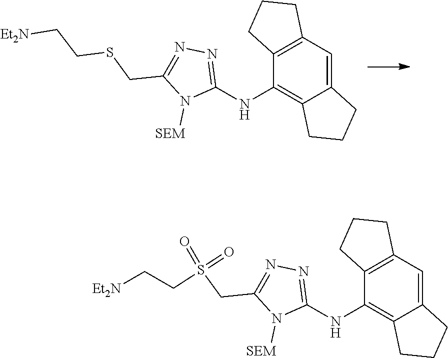

To a solution of 5-(((2-(diethylamino)ethyl)thio)methyl)-N-(1,2,3,5,6,7-hexahydro-s-indacen-4-yl)-((2-(trimethylsilyl)ethoxy)methyl)-4H-1,2,4-triazol-3-amine (0.1 g, 193.86 μmol, 1 eq) in MeOH (4 mL) and H₂O (1 mL) was added Oxone (238 mg, 387.73 μmol, 2 eq). The mixture was stirred at 25° C. for 12 hours. The reaction was quenched with saturated aqueous Na₂SO₃ solution (6 mL) and extracted with CH₂C12 (3×7 mL). The organic phases were dried over anhydrous Na₂SO₄, filtered and concentrated in vacuum. The residue was dissolved in DCM (6 mL), and then to the above solution was added 4,4,5,5-tetramethyl-2-(4,4,5,5-tetramethyl-1,3,2-dioxaborolan-2-yl)-1,3,2-dioxaborolane (98 mg). The mixture was stirred at 25° C. for 2 hours. Then the solution was concentrated in vacuum, and the residue was purified by prep-TLC (SiO₂, dichloromethane:methanol, 5:1) to give the title compound (85 mg, 80.04% yield) as a yellow oil.

¹H NMR (400 MHz, CDCl₃) δ 6.94 (s, 1H), 5.78 (s, 1H), 5.45 (s, 2H), 4.69 (s, 2H), 3.62 (t, 2H), 3.41-3.30 (m, 2H), 3.06-3.02 (m, 2H), 2.89 (t, 4H), 2.78 (t, 4H), 2.60-2.54 (m, 4H), 2.10-2.02 (m, 4H), 1.04 (t, 6H), 0.93 (t, 2H), 0.00 (s, 9H). Single unidentified regioisomer obtained; location of SEM protecting group not determined.

LCMS: m/z 548.5 (M+1)+(ES⁺).

### Intermediate C1: 7-fluoro-5-phenyl-2,3-dihydro-1H-inden-4-amine

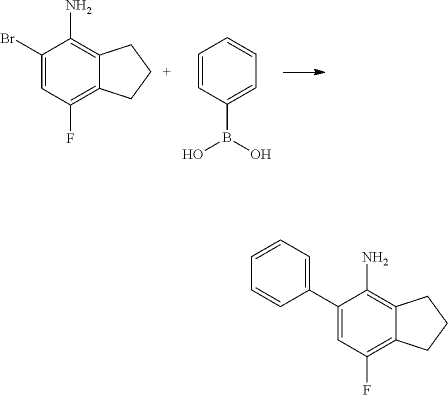

5-Bromo-7-fluoro-2,3-dihydro-1H-inden-4-amine (Intermediate R7, Step E) (0.75 g, 3.26 mmol) was dissolved in dioxane (7 mL). A solution of potassium carbonate (1.352 g, 9.78 mmol) in water (2 mL) was added together with phenylboronic acid (0.417 g, 3.42 mmol). The mixture was degassed with N₂ for 15 min then Pd(dppf)Cl₂. DCM (133 mg, 0.163 mmol) was added. The reaction was heated to 80° C. for 20 h. After cooling to RT, the reaction was partitioned between DCM (30 mL) and water (20 mL). The organic phase was dried (phase separator) and concentrated in vacuo to give a brown oil. The crude product was purified by chromatography on silica gel (24 g column, 0-100% EtOAc/isohexane) to afford the title compound (0.627 g, 84% yield) as a brown oil.

LCMS m/z 228.0 (M+H)⁺ (ES⁺).

¹H NMR (CDCl₃) δ 7.46-7.42 (m, 4H), 7.37-7.33 (m, 1H), 6.73 (d, J=9.2 Hz, 1H), 3.00 (t, J=7.5 Hz, 2H), 2.83 (t, J=7.4 Hz, 2H), 2.20 (p, J=7.6 Hz, 2H). NH₂ not observed.

### Intermediate C2: 4-fluoro-2-isopropyl-6-(2-methoxypyridin-4-yl)aniline

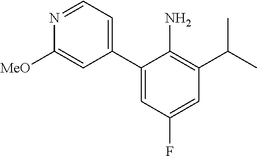 [SMILES: COc1cc(-c2cc(F)cc(C(C)C)c2N)ccn1]

### Step A: 2-bromo-4-fluoro-6-iso-propylaniline

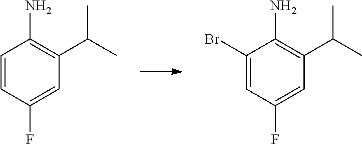

N-Bromosuccinimide (5.64 g, 31.7 mmol) was added portion-wise to 4-fluoro-2-isopropylaniline (4.62 g, 30.2 mmol) in DCM (72 mL) at 0° C. The resulting mixture was stirred at 0° C. for 1 h and then left to warm to RT over 21 h. The reaction mixture was washed with a solution of aqueous sodium hydroxide (2 M, 2×50 mL), dried (MgSO₄), filtered and concentrated in vacuo to give a brown residue. The crude product was then filtered through a plug of silica (50 g) and washed through with 50% DCM in iso-hexane (500 mL). The red filtrate was concentrated to dryness and the crude product was purified by chromatography on silica gel (120 g column, 0-10% DCM/iso-hexane) to afford the title compound (4.99 g, 70% yield) as a red oil.

¹H NMR (CDCl₃) δ 7.07 (dd, 1H), 6.86 (dd, 1H), 4.14 (s, 2H), 2.93 (sept, 1H) and 1.25 (d, 6H).

LCMS; m/z 232.2/234.3 (M+H)⁺ (ES⁺).

### Step B: 4-fluoro-2-isopropyl-6-(2-methoxypyridin-4-yl)aniline

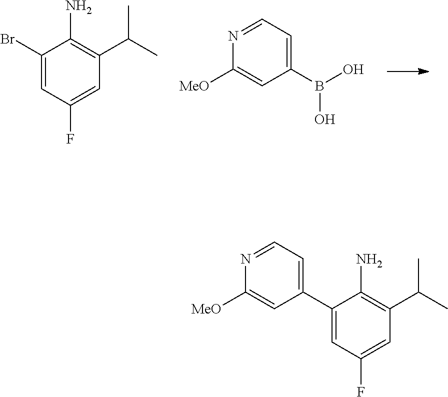 [SMILES: CC(C)c1cc(F)cc(Br)c1N ; COc1cc(B(O)O)ccn1 ; COc1cc(-c2cc(F)cc(C(C)C)c2N)ccn1]

(2-Methoxypyridin-4-yl)boronic acid (144 mg, 0.938 mmol) was added to a stirred, N₂-degassed mixture of 2-bromo-4-fluoro-6-isopropylaniline (200 mg, 0.853 mmol), Pd(dppf)Cl₂ (31.2 mg, 0.043 mmol) and potassium carbonate (354 mg, 2.56 mmol) in 10:11,4-dioxane:water (6.6 mL). The reaction mixture was then heated to 80° C. under a N₂ atmosphere for 22.5 hours. The reaction mixture was left to cool to RT and poured onto EtOAc (10 mL) and water (5 mL). The organic layer was collected and the aqueous layer extracted with EtOAc (2×10 mL). The combined organic layers were dried (Na₂SO₄), filtered and evaporated to dryness. The crude product was purified by chromatography on silica gel (24 g column, 0-50% EtOAc/isohexane) to afford the title compound (174 mg, 78% yield) as a light brown solid.

¹H NMR (CDCl₃) δ 8.25 (d, 1H), 7.00 (dd, 1H), 6.93 (dd, 1H), 6.85 (s, 1H), 6.71 (dd, 1H), 4.01 (s, 3H), 2.92 (sept, 1H) and 1.28 (d, 6H). Exchangeable NH₂ observed as broad signal from 4.5-0.5 ppm.

LCMS m/z 261.1 (M+H)⁺ (ES⁺).

### Intermediate C3: 7-fluoro-5-(pyridin-3-yl)-2,3-dihydro-1H-inden-4-amine

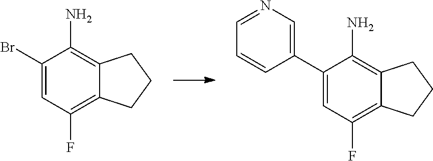 [SMILES: Nc1c(Br)cc(F)c2c1CCC2 ; Nc1c(-c2cccnc2)cc(F)c2c1CCC2]

5-Bromo-7-fluoro-2,3-dihydro-1H-inden-4-amine (Intermediate R7, Step E) (0.45 g, 1.956 mmol) was dissolved in dioxane (5 mL). A solution of K₂CO₃ (0.8 g, 5-79 mmol) in water (1 mL) was added along with pyridin-3-ylboronic acid (0.27 g, 2.197 mmol). The mixture was degassed with N₂ for 15 mins then Pd(dppf)Cl₂.DCM (0.080 g, 0.098 mmol) was added. The reaction mixture was heated to 80° C. (bath temperature) for 18 h. The mixture was cooled to RT and partitioned between DCM (30 mL) and water (10 mL). The organic phase was dried by passing through a hydrophobic frit and concentrated in vacuo to give a brown solid. The crude product was purified by chromatography on silica gel (12 g column, 0-100% EtOAc/isohexane) to afford the title compound (0.317 g, 68%) as a green crystalline solid.

¹H NMR (CDCl₃) δ 8.79 (s, 1H), 8.65 (s, 1H), 8.00 (d, J=7.1 Hz, 1H), 7.56 (s, 1H), 6.71 (d, J=8.9 Hz, 1H), 3.04 (t, J=7.6 Hz, 2H), 2.83 (t, J=7.4 Hz, 2H), 2.24 (p, J=7.5 Hz, 2H), NH₂ not observed.

LCMS; m/z 229.3 (M+H)⁺ (ES⁺).

### Intermediate C4:5-(pyridin-4-yl)-2,3-dihydro-1H-inden-4-amine

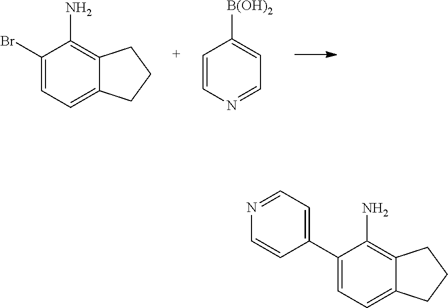 [SMILES: Nc1c(Br)ccc2c1CCC2 ; OB(O)c1ccncc1 ; Nc1c(-c2ccncc2)ccc2c1CCC2]

A solution of 5-bromo-2,3-dihydro-1H-inden-4-amine (Intermediate R4, Step E) (500 mg, 2.36 mmol), pyridin-4-ylboronic acid (348 mg, 2.83 mmol), K₂CO₃ (1.075 g, 7.78 mmol) and Pd(dppf)Cl₂.DCM (385 mg, 0.472 mmol) in 1,4-dioxane (18 mL) and water (3.6 mL) was degassed with N₂ for 10 min. The reaction was stirred at 85° C. for 4 h, cooled to RT and filtered through celite, washing with EtOAc (100 mL). The filtrate was concentrated in vacuo and purified by chromatography on silica gel (24 g column, 0-10% MeOH/DCM) to afford the title compound (400 mg, 77%) as a dark brown solid.

LCMS m/z 211.1 (M+H)⁺ (ES⁺).

¹H NMR (DMSO-d₆) δ 8.62-8.56 (m, 2H), 7.45-7.39 (m, 2H), 6.87 (d, J=7.6 Hz, 1H), 6.60 (d, J=7.6 Hz, 1H), 4.70 (s, 2H), 2.84 (t, J=7.5 Hz, 2H), 2.71 (t, J=7.4 Hz, 2H), 2.03 (p, J=7.5 Hz, 2H).

### Intermediate C5: 5-(2-methoxypyridin-4-yl)-2,3-dihydrobenzofuran-4-amine

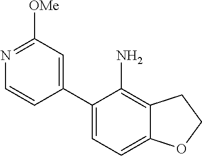 [SMILES: COc1cc(-c2ccc3c(c2N)CCO3)ccn1]

### Step A: N-(5-bromo-2,3-dihydrobenzofuran-4-yl)acetamide

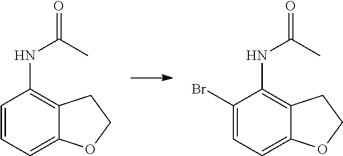

N-(2,3-dihydrobenzofuran-4-yl)acetamide (13.1 g, 73.9 mmol), 4-methylbenzene-sulfonic acid hydrate (7.73 g, 40.7 mmol) and diacetoxypalladium (0.830 g, 3.70 mmol) were suspended in toluene (250 mL) and stirred for 20 min under air atmosphere. NBS (14.47 g, 81 mmol) was added and the mixture was stirred at RT for 30 min. The mixture was diluted with EtOAc (150 mL), washed with NaHCO₃ (100 mL) and Na₂S₂O₃ (10% wt, 100 mL). The aqueous phases were further extracted with DCM (150 mL). The organic phases were combined, dried (MgSO₄), filtered and concentrated under reduced pressure to afford the title compound (22.27 g) which was used crude in the next step.

LCMS; m/z 255.9, 257.9 (M+H)⁺ (ES⁺).

### Step B: 5-bromo-2,3-dihydrobenzofuran-4-amine

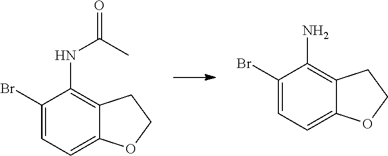

A solution of N-(5-bromo-2,3-dihydrobenzofuran-4-yl)acetamide (22.27 g, 73.9 mmol) in MeOH (400 mL) and conc H₂SO₄ (40 mL) was stirred at reflux for 18 h. The volatiles were removed under reduced pressure, the residue taken up in DCM (300 mL) and basified with aq. NaOH 1 M (100 mL). The organic phase was separated, dried (Na₂SO₄), filtered and concentrated under reduced pressure. The crude product was purified by chromatography on silica gel (220 g column, 0-100% EtOAc/isohexane) to afford the title compound (9.17 g, 57%) as an off-white solid.

¹H NMR (CDCl₃) δ 7.16 (dt, J=8.4, 0.9 Hz, 1H), 6.17 (d, J=8.4 Hz, 1H), 4.61 (t, J=8.7 Hz, 2H), 3.99 (br. s, 2H), 3.05 (t, J=8.7 Hz, 2H).

### Step C: 5-(2-methoxypyridin-4-yl)-2,3-dihydrobenzofuran-4-amine

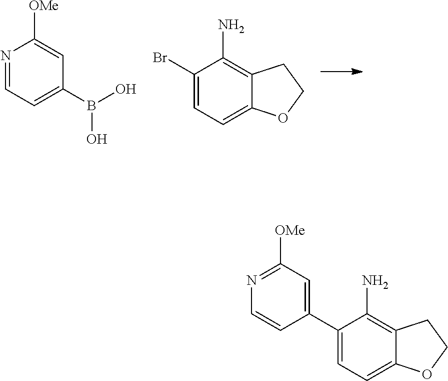 [SMILES: COc1cc(B(O)O)ccn1 ; Nc1c(Br)ccc2c1CCO2 ; COc1cc(-c2ccc3c(c2N)CCO3)ccn1]

Prepared according to the general procedure of 5-(2-methoxypyridin-4-yl)-2,3-dihydro-1H-inden-4-amine (Intermediate R4, Step F) from 5-bromo-2,3-dihydrobenzofuran-4-amine and (2-methoxypyridin-4-yl)boronic acid to afford the title compound (2.25 g, 79%) as an off white solid.

¹H NMR (DMSO-d₆) δ 8.15 (d, J=5.2 Hz, 1H), 6.99 (dd, J=5.3, 1.5 Hz, 1H), 6.84 (d, J=8.2 Hz, 1H), 6.78 (s, 1H), 6.14 (d, J=8.1 Hz, 1H), 4.91 (s, 2H), 4.54 (t, J=8.7 Hz, 2H), 3.87 (s, 3H), 3.01 (t, J=8.7 Hz, 2H).

LCMS; m/z 243.1 (M+H)⁺ (ES⁺).

### Intermediate C6: 7-amino-6-(2-methoxypyridin-4-yl)-2,3-dihydro-1H-indene-4-carbonitrile

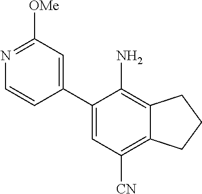 [SMILES: COc1cc(-c2cc(C#N)c3c(c2N)CCC3)ccn1]

### Step A: 7-bromo-5-(2-methoxypyridin-4-yl)-2,3-dihydro-1H-inden-4-amine

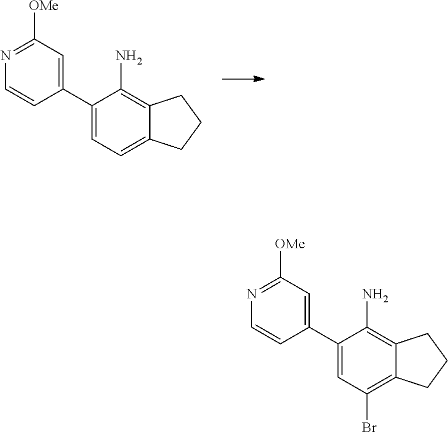 [SMILES: COc1cc(-c2ccc3c(c2N)CCC3)ccn1 ; COc1cc(-c2cc(Br)c3c(c2N)CCC3)ccn1]

NBS (389 mg, 2.185 mmol) was added to a mixture of 5-(2-methoxypyridin-4-yl)-2,3-dihydro-1H-inden-4-amine (Intermediate R4) (500 mg, 2.081 mmol) in CHCl₃ (5 mL) at 0° C. The reaction was stirred at RT for 16 h, washed with 10% Na₂S₂O₃ solution (20 mL), brine (10 mL), dried (MgSO₄) and concentrated in vacuo. The crude was purified by chromatography on silica gel (40 g column, 0-30% EtOAc/isohexane) to afford the title compound (400 mg, 57%) as a tan solid.

LCMS; m/z 318.9/320.9 (M+H)⁺ (ES⁺).

¹H NMR (DMSO-d₆) δ 8.20 (d, J=5.3 Hz, 1H), 7.04-6.97 (m, 2H), 6.80 (d, J=1.3 Hz, 1H), 4.84 (s, 2H), 3.89 (s, 3H), 2.83 (app q, J=7.1 Hz, 4H), 2.06 (p, J=7.6 Hz, 2H).

### Step B: 7-amino-6-(2-methoxypyridin-4-yl)-2,3-dihydro-1H-indene-4-carbonitrile

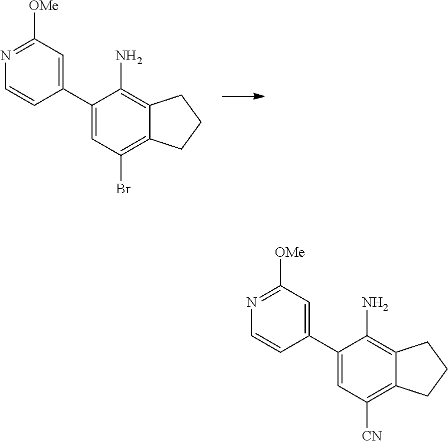 [SMILES: COc1cc(-c2cc(Br)c3c(c2N)CCC3)ccn1 ; COc1cc(-c2cc(C#N)c3c(c2N)CCC3)ccn1]

A solution of 7-bromo-5-(2-methoxypyridin-4-yl)-2,3-dihydro-1H-inden-4-amine (1.75 g, 5.48 mmol) and cyanocopper (0.737 g, 8.22 mmol) in DMF (14 mL) was degassed with N₂. Pd(PPh₃)₄ (0.634 g, 0.548 mmol) was added and the reaction was heated at 100° C. for 18 h. After cooling, the reaction was diluted with EtOAc (100 mL), washed with water (2×10 mL) and sat NaHCO₃ (2×150 mL). The organics were dried (phase separator) and concentrated in vacuo. The crude product was purified by chromatography on silica gel (24 g column, 0-50% EtOAc/isohexane) to afford the title compound (365 mg, 24%) as a brown crystalline solid.

LCMS m/z 266.2 (M+H)⁺ (ES⁺); 264.8 (M−H)⁻ (ES⁻).

¹H NMR (DMSO-d₆) δ 8.20 (d, J=5.2 Hz, 1H), 7.03-6.98 (m, 2H), 6.80 (s, 1H), 4.84 (s, 2H), 3.89 (s, 3H), 2.89-2.79 (m, 4H), 2.06 (p, J=7.5 Hz, 2H).

### Intermediate C7:5-(2-methylpyridin-4-yl)-2,3-dihydro-1H-inden-4-amine

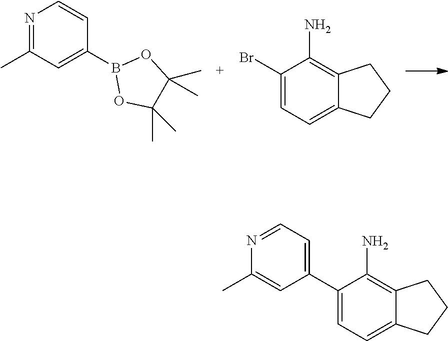

Prepared according to the general procedure of 5-(pyridin-4-yl)-2,3-dihydro-1H-inden-4-amine (Intermediate C4) from 5-bromo-2,3-dihydro-1H-inden-4-amine (Intermediate R4, Step E) and 2-methyl-4-(4,4,5,5-tetramethyl-1,3,2-dioxaborolan-2-yl)pyridine to afford the title compound (153 mg, 46%).

LCMS m/z 225.1 (M+H)⁺ (ES⁺).

¹H NMR (DMSO-d₆) δ 8.49-8.42 (m, 1H), 7.27 (s, 1H), 7.24-7.18 (m, 1H), 6.85 (dd, J=7.7, 3.4 Hz, 1H), 6.59 (dd, J=7.9, 3.4 Hz, 1H), 4.66 (s, 2H), 3.32 (s, 3H), 2.89-2.80 (m, 2H), 2.75-2.68 (m, 2H), 2.08-1.96 (m, 2H).

### Intermediate C8: 5-(2-(difluoromethoxy)pyridin-4-yl)-2,3-dihydro-1H-inden-4-amine

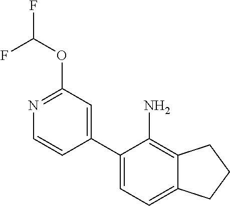 [SMILES: Nc1c(-c2ccnc(OC(F)F)c2)ccc2c1CCC2]

### Step A: 4-bromo-2-(difluoromethoxy)pyridine

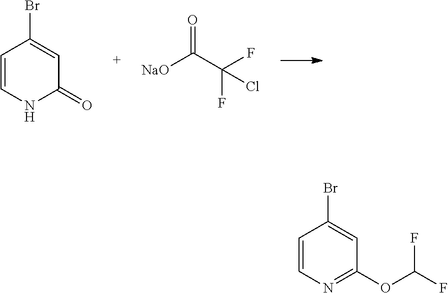

To a solution of sodium 2-chloro-2,2-difluoroacetate (12.38 g, 81 mmol) in MeCN (400 mL) was added 4-bromopyridin-2 (1H)-one (10.09, 58 mmol) and the reaction was refluxed for 20 h. The mixture was filtered and the solid purified by chromatography on silica gel (80 g column, 0-10% EtOAc/isohexane) to afford the title compound (6.75 g, 51%) as a clear colourless liquid.

¹H NMR (DMSO-d₆) δ 8.1 (d, J=5.5 Hz, 1H), 7.86-7.52 (m, 2H), 7.49 (d, J=1.6 Hz, 1H).

### Step B: 2-(difluoromethoxy)-4-(4,4,5,5-tetramethyl-1,3,2-dioxaborolan-2-yl)pyridine

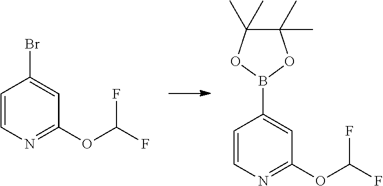 [SMILES: FC(F)Oc1cc(Br)ccn1 ; CC1(C)OB(c2ccnc(OC(F)F)c2)OC1(C)C]

A solution of 4-bromo-2-(difluoromethoxy)pyridine (1.25 g, 5.56 mmol) in 1,4-dioxane (30 mL) was added to Pd(dppf)Cl₂ (406 mg, 0.56 mmol), B₂Pin₂ (1.55 g, 6.10 mmol) and KOAc (1.2 g, 12.2 mmol) under N₂ and the reaction was heated to reflux for 22 h. The mixture was filtered through celite and concentrated in vacuo to afford the crude title compound (2.57 g) which was used without further purification.

LCMS m/z 271.8 (M+H)⁺ (ES⁺).

¹H NMR (DMSO-d₆): δ 8.28 (d, J=4.9 Hz, 1H), 7.70 (t, J=72.9 Hz, 1H), 7.41 (d, J=4.8 Hz, 1H), 7.13 (s, 1H), 1.29 (s, 12H).

### Step C: 5-(2-(difluoromethoxy)pyridin-4-yl)-2,3-dihydro-1H-inden-4-amine

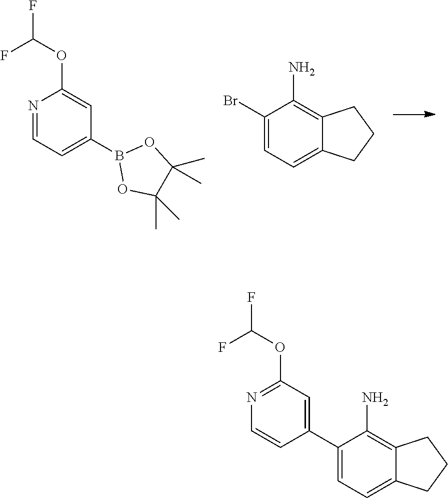 [SMILES: CC1(C)OB(c2ccnc(OC(F)F)c2)OC1(C)C ; Nc1c(Br)ccc2c1CCC2 ; Nc1c(-c2ccnc(OC(F)F)c2)ccc2c1CCC2]

Prepared according to the general procedure of 5-(pyridin-4-yl)-2,3-dihydro-1H-inden-4-amine (Intermediate C4) from 5-bromo-2,3-dihydro-1H-inden-4-amine (Intermediate R4, Step E) and 2-(difluoromethoxy)-4-(4,4,5,5-tetramethyl-1,3,2-dioxaborolan-2-yl)pyridine to afford the title compound (0.227 g, 83%) as a colourless crystalline solid.

LCMS m/z 277.1 (M+H)⁺ (ES⁺); 275.2 (M−H)⁻ (ES⁻).

¹H NMR (DMSO-d₆) δ 8.27 (d, J=5.2 Hz, 1H), 7.75 (t, J=73.1 Hz, 1H), 7.33-7.29 (m, 1H), 7.06 (s, 1H), 6.89 (d, J=7.6 Hz, 1H), 6.60 (d, J=7.7 Hz, 1H), 4.80 (s, 2H), 2.83 (t, J=7.5 Hz, 2H), 2.71 (t, J=7.4 Hz, 2H), 2.03 (p, J=7.6 Hz, 2H).

### Intermediate C9: 4-fluoro-2-isopropyl-6-(2-methylpyridin-4-yl)aniline

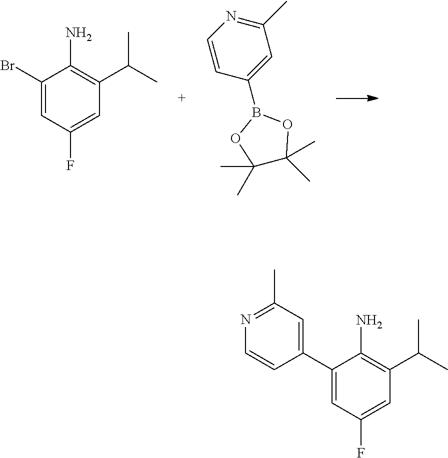 [SMILES: CC(C)c1cc(F)cc(Br)c1N ; Cc1cc(B2OC(C)(C)C(C)(C)O2)ccn1 ; Cc1cc(-c2cc(F)cc(C(C)C)c2N)ccn1]

Prepared according to the general method of 5-(pyridin-4-yl)-2,3-dihydro-1H-inden-4-amine (Intermediate C4) from 2-bromo-4-fluoro-6-isopropylaniline (Intermediate C2, Step A) and (2-methylpyridin-4-yl)boronic acid to afford the title compound (130 mg, 62%) as a light brown solid.

LCMS m/z 245.1 (M+H)⁺ (ES⁺).

¹H NMR (CDCl₃) δ 8.57 (dd, 1H), 7.29 (d, 1H), 7.25-7.22 (m, 1H), 6.93 (dd, 1H), 6.70 (dd, 1H), 3.62 (br s, 2H), 2.92 (sept, 1H), 2.64 (s, 3H) and 1.29 (d, 6H).

### Intermediate C10: 7-fluoro-5-(2-methoxypyridin-4-yl)-2,3-dihydro-1H-inden-4-amine

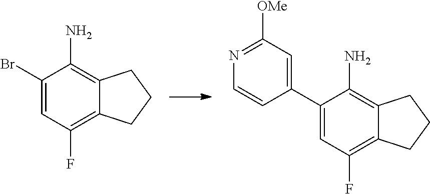 [SMILES: Nc1c(Br)cc(F)c2c1CCC2 ; COc1cc(-c2cc(F)c3c(c2N)CCC3)ccn1]

Prepared according to the general procedure of 5-(pyridin-4-yl)-2,3-dihydro-1H-inden-4-amine (Intermediate C4) from 5-bromo-7-fluoro-2,3-dihydro-1H-inden-4-amine (Intermediate R7, Step E) and (2-methoxypyridin-4-yl)boronic acid to afford the title compound (0.185 g, 49%) as a pale brown oil that crystallized on standing.

LCMS m/z 259.3 (M+H)⁺ (ES⁺).

¹H NMR (CDCl₃) δ 8.27 (d, J=5.4 Hz, 1H), 7.06 (d, J=5.3 Hz, 1H), 6.95 (s, 1H), 6.73 (d, J=9.0 Hz, 1H), 4.03 (s, 3H), 3.00 (t, J=7.5 Hz, 2H), 2.85 (t, J=7.4 Hz, 2H), 2.23 (p, J=7.5 Hz, 2H), Two exchangeable protons not observed.

### Intermediate C11: 8-amino-1,2,3,5,6,7-hexahydro-s-indacene-4-carbonitrile

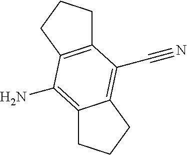 [SMILES: N#Cc1c2c(c(N)c3c1CCC3)CCC2]

### Step A: 8-bromo-1,2,3,5,6,7-hexahydro-s-indacen-4-amine

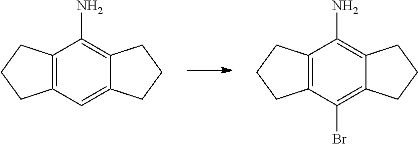 [SMILES: Nc1c2c(cc3c1CCC3)CCC2 ; Nc1c2c(c(Br)c3c1CCC3)CCC2]

To a solution of 1,2,3,5,6,7-hexahydro-s-indacen-4-amine (0.5 g, 2.89 mmol) in DCM (10 mL) at 0° C. was added NBS (0.514 g, 2.89 mmol). The solution was gradually warmed to RT and stirred for 12 h. The reaction mixture was diluted with Na₂S₂O₃ (25 mL) and extracted with DCM (2×20 ml). The organic phase was washed with water (10 mL) and brine (20 mL). The organic phase was dried (MgSO₄), filtered and concentrated in vacuo to afford a brown solid. The crude product was purified by chromatography on silica gel (12 g column, 0-10% EtOAc/iso-hexane) to afford the title compound (579 mg, 79%) as a brown solid.

LCMS; m/z 252/254 (M+H)⁺ (ES⁺).

¹H NMR (DMSO-d₆) δ 4.71 (s, 2H), 2.80-2.63 (m, 8H), 2.08-1.91 (m, 4H).

### Step B: 8-amino-1,2,3,5,6,7-hexahydro-s-indacene-4-carbonitrile

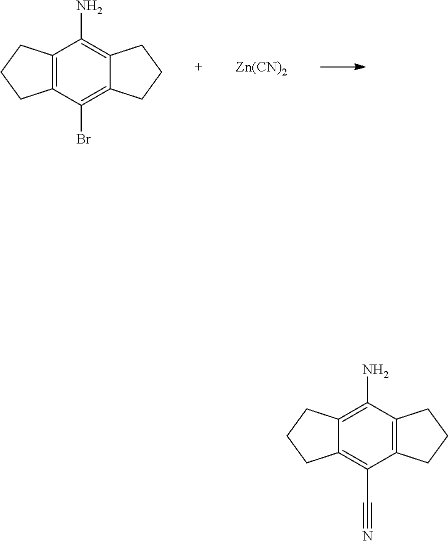

A solution of 8-bromo-1,2,3,5,6,7-hexahydro-s-indacen-4-amine (579 mg, 2.296 mmol) and dicyanozinc (283 mg, 2.411 mmol) in DMA (10 mL) was degassed for 10 min with N₂, then Pd(PPh₃)₄ (265 mg, 0.230 mmol) was added and the reaction mixture was then heated to 100° C. for 18 h. The reaction mixture was allowed to cool to RT and then filtered over Celite eluting with EtOAc (30 mL). The filtrate was washed with sat aq NaHCO₃ (2×10 mL), water (2×10 mL) and brine (2×10 mL). The organic layer was then dried (Na₂SO₄) and concentrated in vacuo. The residue was purified by chromatography on silica gel (24 g column, 0-40% EtOAc/isohexane) to afford the title compound (96 mg, 20%) as colourless solid.

LCMS m/z 199.1 (M+H)⁺ (ES⁺).

¹H NMR (DMSO-d₆) δ 5.68 (s, 2H), 2.85 (t, J=7.5 Hz, 4H), 2.64 (t, J=7.4 Hz, 4H), 2.15-1.96 (m, 4H).

### Intermediate C12: 8-fluoro-1,2,3,5,6,7-hexahydro-s-indacen-4-amine

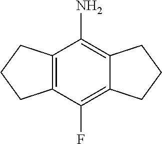 [SMILES: Nc1c2c(c(F)c3c1CCC3)CCC2]

### Step A: N-(1,2,3,5,6,7-hexahydro-s-indacen-4-yl)acetamide

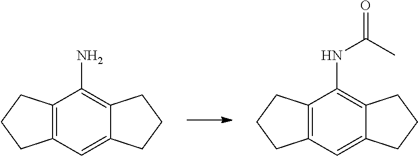 [SMILES: Nc1c2c(cc3c1CCC3)CCC2 ; CC(=O)Nc1c2c(cc3c1CCC3)CCC2]

Acetic anhydride (6.00 mL, 63.5 mmol) was added dropwise to a solution of 1,2,3,5,6,7-hexahydro-s-indacen-4-amine (10 g, 57.7 mmol) and Et₃N (9.65 mL, 69.3 mmol) in DCM (140 mL) at 0° C. The solution was stirred at RT overnight. Water (100 mL) was added and the solid collected by filtration, washed with water and dried in vacuo to afford the title compound (9.63 g, 77%) as an off-white solid.

¹H NMR (DMSO-d₆) δ 9.31 (s, 1H), 6.94 (s, 1H), 2.81 (t, J=7.4 Hz, 4H), 2.67 (t, J=7.4 Hz, 4H), 2.00 (s, 3H), 1.96 (p, J=7.4 Hz, 4H).

### Step B: N-(8-fluoro-1,2,3,5,6,7-hexahydro-s-indacen-4-yl)acetamide

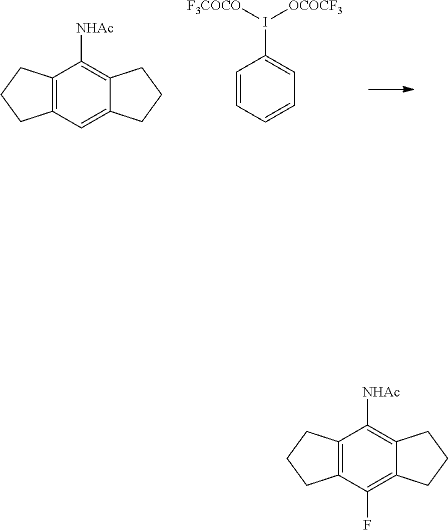

A solution of N-(1,2,3,5,6,7-hexahydro-s-indacen-4-yl)acetamide (4.0 g, 18.6 mmol) and HF-pyridine (20 mL, 222 mmol) in DCM (13 mL) was cooled in an ice bath. A solution of PhI(OCOCF₃)₂ (12 g, 27.9 mmol) in DCM (13 mL) was added dropwise and the reaction was stirred in an ice bath for 1 h. The reaction mixture was quenched with sat aq calcium hydroxide and the phases were separated. The organics were passed through a hydrophobic frit and the solvent was removed in vacuo. The crude product was split into 2 batches and purified by chromatography on silica gel (220 g and 120 g column, 0-100% EtOAc/iso-hexane) to afford the title compound (747 mg, 16%) as a pale yellow solid.

¹H NMR (DMSO-d₆) δ 9.32 (br s, 1H), 2.84 (t, J=7.5 Hz, 4H), 2.71 (t, J=7.5 Hz, 4H), 2.03 (p, J=7.5 Hz, 4H), 1.99 (3H, s). ¹⁹F NMR (471 MHz, DMSO-d₆) δ−125.83.

### Step C: 8-fluoro-1,2,3,5,6,7-hexahydro-s-indacen-4-amine

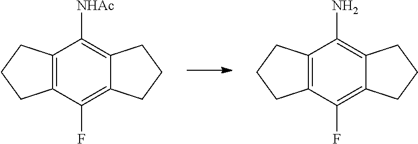 [SMILES: CC(=O)Nc1c2c(c(F)c3c1CCC3)CCC2 ; Nc1c2c(c(F)c3c1CCC3)CCC2]

A solution of N-(8-fluoro-1,2,3,5,6,7-hexahydro-s-indacen-4-yl)acetamide (0.747 g, 3.20 mmol) in EtOH (14 mL) and conc. HCl (14 mL) was heated to reflux. The solution was cooled to RT and 2 N NaOH (20 mL) was added. The product was extracted with DCM (3×50 mL) and the organic extracts were passed through a hydrophobic frit and the solvent removed in vacuo. The crude product was purified by chromatography on silica gel (24 g column, 0-50% EtOAc/iso-hexane) to afford the title compound (0.216 g, 35%) as a pale brown solid.

LCMS; m/z 192.4 (M+H)⁺ (ES⁺).

¹H NMR (DMSO-d₆) δ 4.41 (br s, 2H), 2.75 (t, J=7.5 Hz, 4H), 2.62 (t, J=7.5 Hz, 4H), 2.02 (p, J=7.5 Hz, 4H).

### Intermediate C1A: 5-(2-methoxypyridin-4-yl)-7-(oxazol-2-yl)-2,3-dihydro-1H-inden-4-amine

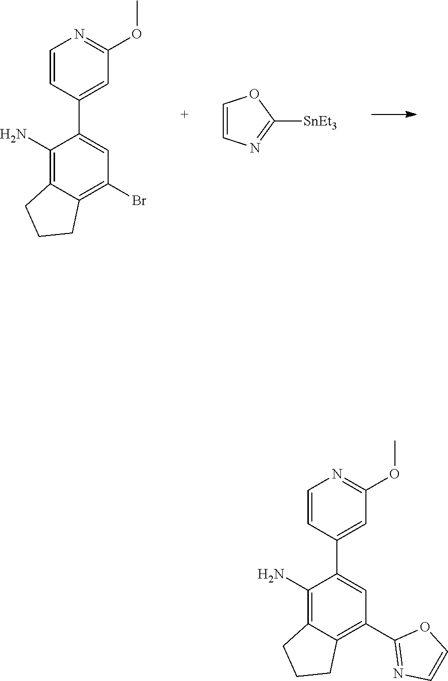

Pd(PPh₃)₄ (217 mg, 0.188 mmol) was added to a degassed solution of 7-bromo-5-(2-methoxypyridin-4-yl)-2,3-dihydro-1H-inden-4-amine (Intermediate C6, Step A) (600 mg, 1.88 mmol) and 2-(tributylstannyl)oxazole (1.35 g, 3-76 mmol) in dioxane (10 mL). The reaction was heated at 85° C. for 20 h and concentrated in vacuo. The residue was taken up in isohexane (10 mL), extracted with MeCN (2×100 mL), filtered through a plug of silica and concentrated in vacuo. The crude product was purified by chromatography on silica gel (40 g column, 0-100% EtOAc/isohexane) to afford the title compound (502 mg, 83%) as a tan solid.

LCMS m/z 308.2 (M+H)⁺ (ES⁺).

¹H NMR (DMSO-d₆) δ 8.23 (dd, J=5.3, 0.7 Hz, 1H), 8.05 (d, J=0.8 Hz, 1H), 7.49 (s, 1H), 7.26 (d, J=0.8 Hz, 1H), 7.04 (dd, J=5.2, 0.5 Hz, 1H), 6.84 (m, 1H), 5.24 (s, 2H), 3.89 (s, 3H), 3.24 (t, J=7.6 Hz, 2H), 2.75 (t, J=7.5 Hz, 2H), 2.08 (p, J=7.5 Hz, 2H).

### Intermediate C14: 1-(8-amino-1,2,3,5,6,7-hexahydro-s-indacen-4-yl)ethanone

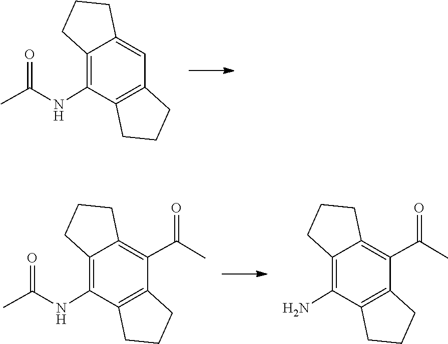 [SMILES: CC(=O)Nc1c2c(cc3c1CCC3)CCC2 ; CC(=O)Nc1c2c(c(C(C)=O)c3c1CCC3)CCC2 ; CC(=O)c1c2c(c(N)c3c1CCC3)CCC2]

Acetyl chloride (1.998 mL, 28.1 mmol) was added dropwise to N-(1,2,3,5,6,7-hexahydro-s-indacen-4-yl)acetamide (Intermediate C12, Step A) (2.42 g, 11.24 mmol) in DCM (25 mL) at 0° C. AlCl₃ (3.75 g, 28.1 mmol) was then added portionwise over 10 min and the reaction stirred at ° C. for 15 min and then at RT for 4 h. The reaction mixture was poured onto ice and allowed to stir for 1 h. HCl (100 mL) was added, the mixture was extracted with EtOAc (300 mL) and the organic phase washed with brine (200 mL), dried (MgSO₄) and concentrated in vacuo. The crude was stirred in conc. HCl (20 mL) and water (20 mL) at 120° C. for 16 h. After cooling, the reaction was diluted with water (40 mL) and poured into a stirred solution of 2M NaOH (60 mL). Sat aq NaHCO₃ was then added until neutral pH. The aqueous mixture was extracted with DCM (2×150 mL). The organics were dried (phase separator) and concentrated in vacuo to afford the title compound (1.4 g, 58%) as a solid.

LCMS m/z 216.2 (M+H)⁺ (ES⁺).

¹H NMR (DMSO-d₆) δ 5.33 (br s, 2H), 3.01 (t, J=7.4 Hz, 4H), 2.60 (t, J=7.5 Hz, 4H), 2.36 (s, 3H), 1.98 (p, J=7.5 Hz, 4H).

### Intermediate C15: 5-(2-cyclopropylpyridin-4-yl)-2,3-dihydro-1H-inden-4-amine

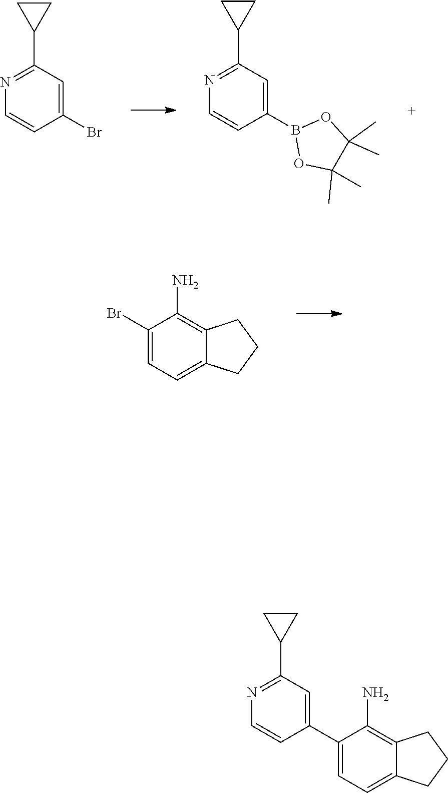 [SMILES: Brc1ccnc(C2CC2)c1 ; CC1(C)OB(c2ccnc(C3CC3)c2)OC1(C)C ; Nc1c(Br)ccc2c1CCC2 ; Nc1c(-c2ccnc(C3CC3)c2)ccc2c1CCC2]

To a solution of 4-bromo-2-cyclopropylpyridine (1.0 g, 5.05 mmol) in dioxane (25 mL) was added B₂Pin₂ (1.41 g, 5.55 mmol) and KOAc (1.982 g, 20.20 mmol). The reaction mixture was heated to 6° C. and degassed with N₂. PdCl₂(dppf).DCM (0.206 g, 0.252 mmol) was added and the reaction heated at 100° C. for 2 h. After cooling, a solution of 5-bromo-2,3-dihydro-1H-inden-4-amine (Intermediate R4, Step E) (1.071 g, 5.05 mmol) in dioxane (5 mL) was added followed by a solution of K₂CO₃ (2.79 g, 20.20 mmol) in water (2 mL). The reaction was heated at 90° C. for 2 h. After cooling, the reaction was partitioned between EtOAc (50 mL) and brine (20 mL). The phases were separated and the organics dried and the solvent was removed in vacuo.

The product was purified by chromatography on silica gel (24 g column, 0-80% EtOAc/isohexane) to afford the title compound (1.19 g, 89%).

LCMS m/z 251.2 (M+H)⁺ (ES⁺).

¹H NMR (DMSO-d₆) δ 8.40 (d, J=5.0 Hz, 1H), 7.31-7.28 (m, 1H), 7.15 (dd, J=5.1, 1.7 Hz, 1H), 6.86 (d, J=7.6 Hz, 1H), 6.59 (d, J=7.6 Hz, 1H), 4.64 (br s, 2H), 2.83 (t, J=7.5 Hz, 2H), 2.71 (t, J=7.3 Hz, 2H), 2.12 (tt, J=7.3, 5.5 Hz, 1H), 2.06-2.00 (m, 2H), 0.97-0.92 (m, 4H).

### Intermediate C16: 5-(1-methyl-1H-pyrazol-4-yl)-2,3-dihydro-1H-inden-4-amine

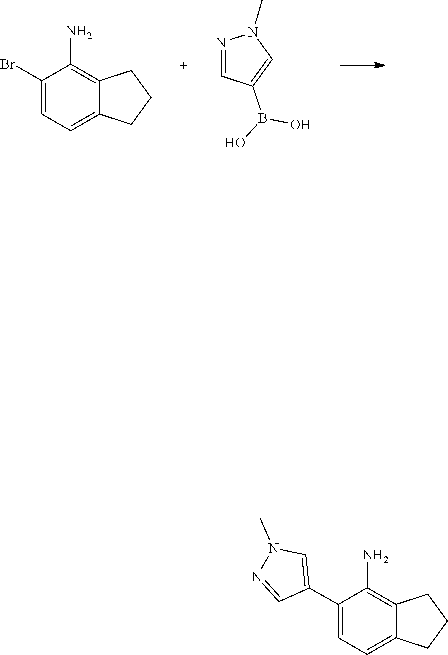

Prepared according to the general procedure of 5-(pyridin-4-yl)-2,3-dihydro-1H-inden-4-amine (Intermediate C4) from 5-bromo-2,3-dihydro-1H-inden-4-amine (Intermediate R4, Step E) and (1-methyl-H-pyrazol-4-yl)boronic acid to afford the title compound (188 mg, 23%) as a brown oil.

LCMS m/z 214.2 (M+H)⁺ (ES⁺).

¹H NMR (DMSO-d₆) δ 7.86 (s, 1H), 7.57 (s, 1H), 6.92 (d, J=7.6 Hz, 1H), 6.52 (d, J=7.6 Hz, 1H), 4.50 (s, 2H), 3.87 (s, 3H), 2.80 (t, J=7.5 Hz, 2H), 2.69 (t, J=7.3 Hz, 2H), 2.04-1.97 (m, 2H).

### Intermediate C17: 5-(1-methyl-1H-pyrazol-5-yl)-2,3-dihydro-1H-inden-4-amine

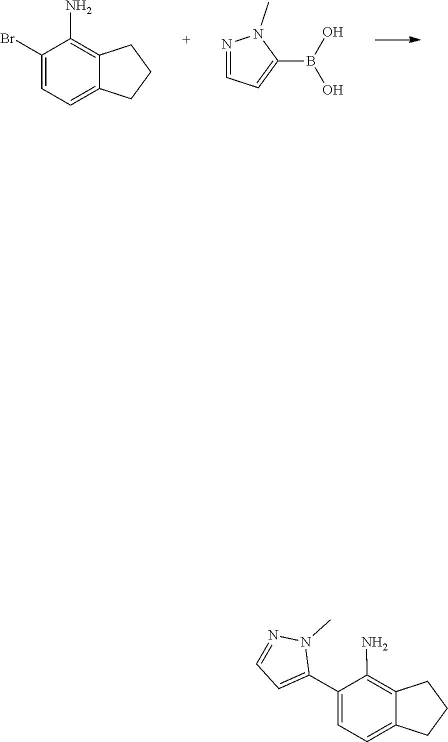

Prepared according to the general procedure of 5-(pyridin-4-yl)-2,3-dihydro-1H-inden-4-amine (Intermediate C4) from 5-bromo-2,3-dihydro-1H-inden-4-amine (Intermediate R4, Step E) and (1-methyl-1H-pyrazol-5-yl)boronic acid to afford the title compound (28 mg, 3%) as a brown oil.

LCMS m/z 214.3 (M+H)⁺ (ES⁺).

¹H NMR (DMSO-d₆) δ 7.49 (d, J=1.8 Hz, 1H), 6.81 (d, J=7.5 Hz, 1H), 6.57 (d, J=7.5 Hz, 1H), 6.22 (d, J=1.8 Hz, 1H), 4.54 (s, 2H), 3.63 (s, 3H), 2.84 (t, J=7.5 Hz, 2H), 2.71 (t, J=7.4 Hz, 2H), 2.07-1.99 (m, 2H).

### Intermediate C18: 5-(tetrahydro-2H-pyran-4-yl)-2,3-dihydro-1H-inden-4-amine

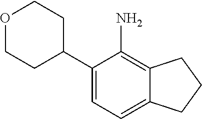 [SMILES: Nc1c(C2CCOCC2)ccc2c1CCC2]

### Step A: 5-(3,6-dihydro-2H-pyran-4-yl)-2,3-dihydro-1H-inden-4-amine

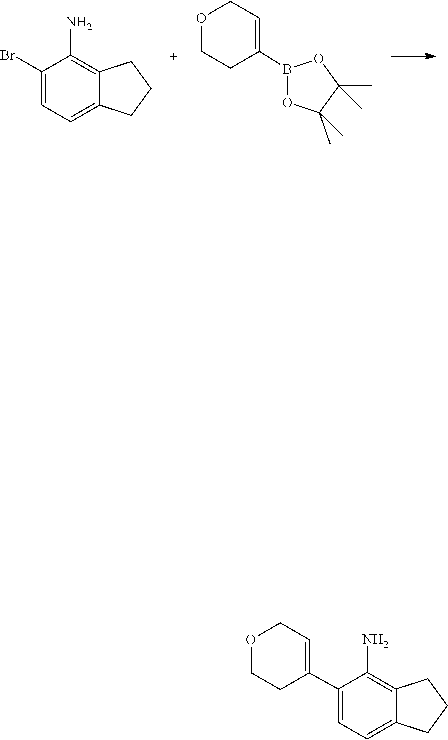 [SMILES: Nc1c(Br)ccc2c1CCC2 ; CC1(C)OB(C2=CCOCC2)OC1(C)C ; Nc1c(C2=CCOCC2)ccc2c1CCC2]

Prepared according to the general procedure of 5-(pyridin-4-yl)-2,3-dihydro-1H-inden-4-amine (Intermediate C4) from 5-bromo-2,3-dihydro-1H-inden-4-amine (Intermediate R4, Step E) and 2-(3,6-dihydro-2H-pyran-4-yl)-4,4,5,5-tetramethyl-1,3,2-dioxaborolane to afford the title compound (373 mg, 45%) as a brown oil.

LCMS m/z 216.2 (M+H)⁺ (ES⁺).

¹H NMR (DMSO-d₆) δ 6.71 (d, J=7.5 Hz, 1H), 6.46 (d, J=7.6 Hz, 1H), 5.72-5.64 (m, 1H), 4.18 (q, J=2.7 Hz, 2H), 3.82 (t, J=5.4 Hz, 2H), 2.77 (t, J=7.5 Hz, 2H), 2.65 (t, J=7.3 Hz, 2H), 2.28-2.23 (m, 2H), 1.98 (p, J=7.4 Hz, 2H). Two exchangeable protons not observed.

### Step B: 5-(tetrahydro-2H-pyran-4-yl)-2,3-dihydro-1H-inden-4-amine

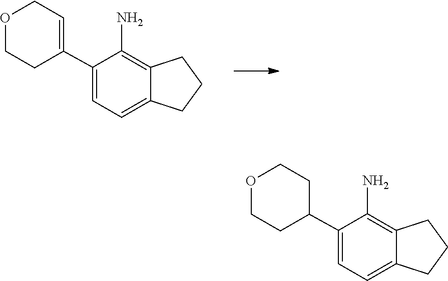 [SMILES: Nc1c(C2=CCOCC2)ccc2c1CCC2 ; Nc1c(C2CCOCC2)ccc2c1CCC2]

A mixture of 5-(3,6-dihydro-2H-pyran-4-yl)-2,3-dihydro-1H-inden-4-amine (373 mg, 1.73 mmol) and 10% Pd/C (20 g) in EtOAc (10 mL) was hydrogenated at 4 bars for 4 h. The mixture was filtered and volatiles evaporated to afford the title compound (303 mg, 80%) as an oil.

LCMS m/z 218.2 (M+H)⁺ (ES⁺).

¹H NMR (DMSO-d₆) δ 6.78 (d, J=7.7 Hz, 1H), 6.46 (d, J=7.6 Hz, 1H), 4.61 (s, 2H), 3.95-3.89 (m, 2H), 3.49 (td, J=11.6, 2.0 Hz, 2H), 2.85 (tt, J=11.8, 3.7 Hz, 1H), 2.75 (t, J=7.5 Hz, 2H), 2.64 (t, J=7.3 Hz, 2H), 1.97 (p, J=7.4 Hz, 2H), 1.66-1.61 (m, 2H), 1.59-1.49 (m, 2H).

### Intermediate C19: 2-cyclopropyl-4-fluoro-6-(2-methoxypyridin-4-yl)aniline

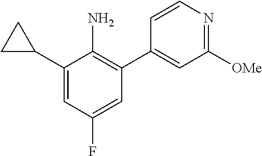 [SMILES: COc1cc(-c2cc(F)cc(C3CC3)c2N)ccn1]

### Step A: 2-bromo-4-fluoro-6-(2-methoxypyridin-4-yl)aniline

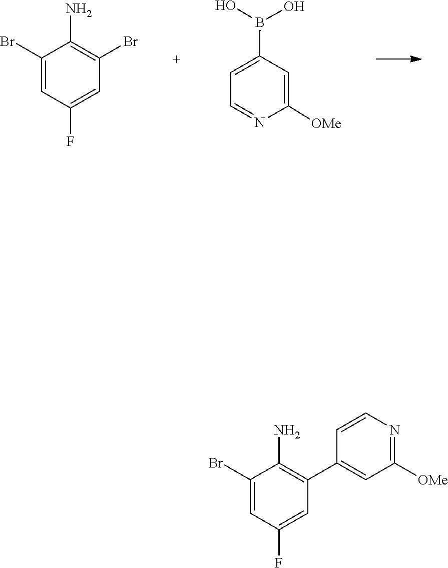 [SMILES: Nc1c(Br)cc(F)cc1Br ; COc1cc(B(O)O)ccn1 ; COc1cc(-c2cc(F)cc(Br)c2N)ccn1]

2,6-Dibromo-4-fluoroaniline (10 g, 37.2 mmol) and (2-methoxypyridin-4-yl)boronic acid (5.7 g, 37.3 mmol) were suspended in a mixture of toluene (350 mL) and EtOH (100 mL). A solution of Na₂CO₃ (24 g, 226 mmol) in water (100 mL) was added and the mixture was degassed at 50° C. with N₂. Pd(Ph₃P)₄ (2.1 g, 1.817 mmol) was added in a single portion and the mixture was heated to 90° C. overnight. After cooling, the mixture was diluted with water (200 mL) and the aqueous phase was extracted with EtOAc (5×100 mL). The combined organics were dried (MgSO₄) and concentrated in vacuo. The crude product was purified by chromatography on silica gel (220 g column, 0-100% EtOAc/DCM) to afford the title compound (5.04 g, 45%) as a pale brown solid.

LCMS m/z 297.1/299.2 (M+H)⁺ (ES⁺).

¹H NMR (CDCl₃) δ 8.29 (dd, J=5.3, 0.7 Hz, 1H), 7.27 (dd, J=7.8, 2.9 Hz, 1H), 6.99 (dd, J=5.3, 1.5 Hz, 1H), 6.87 (dd, J=8.6, 3.1 Hz, 1H), 6.85 (s, 1H), 4.11 (br s, 2H), 4.03 (s, 3H).

### Step B: 2-bromo-4-fluoro-6-(2-methoxypyridin-4-yl)aniline

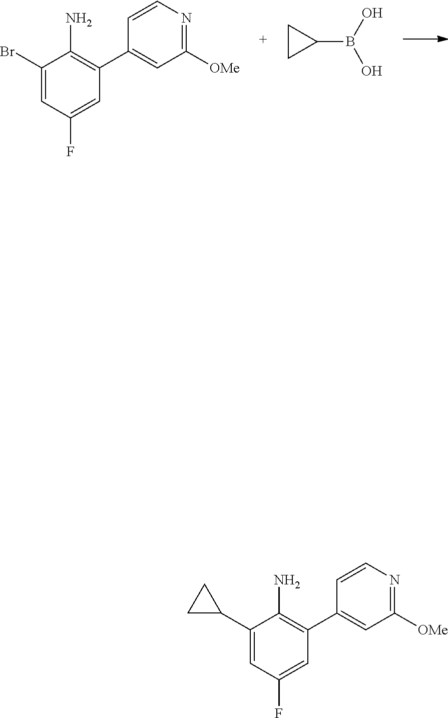 [SMILES: COc1cc(-c2cc(F)cc(Br)c2N)ccn1 ; OB(O)C1CC1 ; COc1cc(-c2cc(F)cc(C3CC3)c2N)ccn1]

2-Bromo-4-fluoro-6-(2-methoxypyridin-4-yl)aniline (0.5 g, 1.683 mmol), potassium phosphate (1.0 g, 4.71 mmol), cyclopropylboronic acid (250 mg, 2.91 mmol), and tricyclohexylphosphine (60 mg, 0.214 mmol) were suspended in a mixture of toluene (10 mL) and water (2 mL). The mixture was degassed for 15 min before Pd(OAc)₂ (25 mg, 0.11 mmol) was added. The mixture was heated to 90° C. overnight. The mixture was cooled to RT and partitioned between DCM (25 mL) and water (10 mL). The organic phase was dried (phase separator) and concentrated in vacuo. The crude product was purified by chromatography on silica gel (12 g column, 0-50% EtOAc/isohexane) to afford the title compound (201 mg, 44%) as a thick brown oil that crystallized on standing.

LCMS m/z 259.3 (M+H)⁺ (ES⁺).

¹H NMR (DMSO-d₆) δ 8.23 (dd, J=5-3, 0.7 Hz, 1H), 7.06 (dd, J=5.3, 1.5 Hz, 1H), 6.85 (dd, J=1.5, 0.8 Hz, 1H), 6.80-6.75 (m, 2H), 4.64 (s, 2H), 3.90 (s, 3H), 1.80-1.72 (m, 1H), 0.97-0.88 (m, 2H), 0.62-0.55 (m, 2H).

### Intermediate C20: 5-(2-methoxypyridin-4-yl)-6-methyl-2,3-dihydro-1H-inden-4-amine

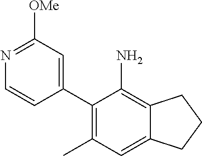 [SMILES: COc1cc(-c2c(C)cc3c(c2N)CCC3)ccn1]

### Step A: N-(6-bromo-4-nitro-2,3-dihydro-1H-inden-5-yl)acetamide

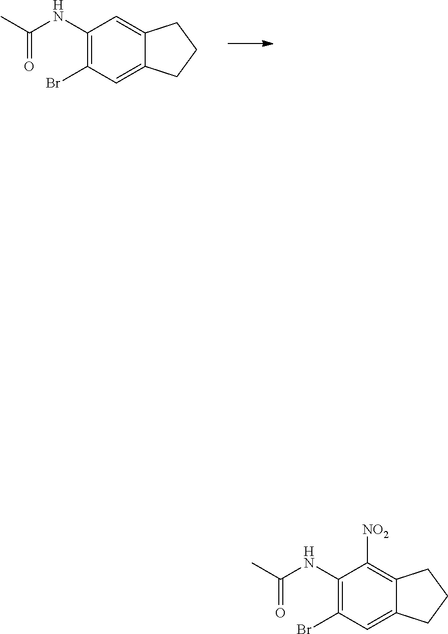 [SMILES: CC(=O)Nc1cc2c(cc1Br)CCC2 ; CC(=O)Nc1c(Br)cc2c(c1[N+](=O)[O-])CCC2]

Nitric acid (70% wt, 9 mL, 141 mmol) was added to a solution of N-(6-bromo-2,3-dihydro-1H-inden-5-yl)acetamide (12.2 g, 48.0 mmol) in AcOH (120 mL). The reaction was stirred at RT for 18 h then heated at 50° C. for 2 h. After cooling, an additional portion of nitric acid (50 mL) was added and the reaction was stirred at RT for 24 h and poured onto ice-water (500 mL. The solid was filtered, washed with water (200 mL) and dissolved in DCM (300 mL). The organics were dried (MgSO₄) and evaporated in vacuo. The residue was triturated with EtOAc, filtered, washed with isohexane and dried to afford the title compound (6.75 g, 45%) as a solid.

LCMS m/z 299/301 (M+H)⁺ (ES⁺); 297/299 (M−H)⁻ (ES⁻).

¹H NMR (CDCl₃) δ 7.67 (s, 1H), 7.40 (s, 1H), 3.11 (t, J=7.2 Hz, 2H), 3.01 (t, J=7.4 Hz, 2H), 2.23 (s, 3H), 2.21-2.14 (m, 2H).

### Step B: N-(6-methyl-4-nitro-2,3-dihydro-1H-inden-5-yl)acetamide

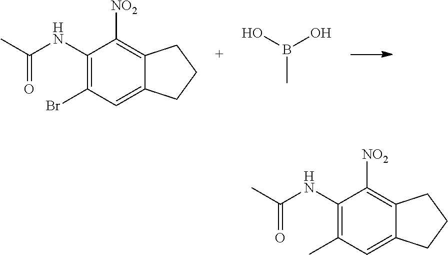

A mixture of N-(6-bromo-4-nitro-2,3-dihydro-1H-inden-5-yl)acetamide (1.0 g, 3.34 mmol), methylboronic acid (0.26 g, 4.35 mmol), K₂CO₃ (1.386 g, 10.03 mmol), Pd(PPh₃)₄ (0.386 g, 0.334 mmol), water (3 mL) and dioxane (30 mL), was degassed with N₂ and heated at 100° C. overnight. The mixture was diluted with EtOAc (50 mL) and washed with water (20 mL) and brine (20 mL). The organic phase was dried (MgSO₄) and concentrated in vacuo. The crude product was purified by chromatography on silica gel (12 g column, 0-10% MeOH/DCM) to afford the title compound (186 mg, 24%) as a colourless solid.

LCMS m/z 235.0 (M+H)⁺ (ES⁺).

¹H NMR (DMSO-d₆) δ 9.66 (s, 1H), 7.41 (s, 1H), 2.97-2.89 (m, 4H), 2.21 (s, 3H), 2.06 (p, J=7.5 Hz, 2H), 2.00 (s, 3H).

### Step C: 6-methyl-4-nitro-2,3-dihydro-1H-inden-5-amine

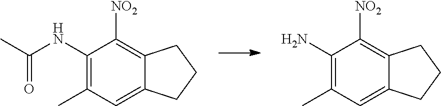 [SMILES: CC(=O)Nc1c(C)cc2c(c1[N+](=O)[O-])CCC2 ; Cc1cc2c(c([N+](=O)[O-])c1N)CCC2]

N-(6-methyl-4-nitro-2,3-dihydro-1H-inden-5-yl)acetamide (570 mg, 2.433 mmol) was stirred in conc HCl (10 mL) and water (10 mL) at 120° C. for 16 h. After cooling the reaction was diluted with water (40 mL) and poured into a stirred solution of 2 M NaOH (60 mL). Sat aq NaHCO₃ was added until neutral pH. The aqueous mixture was extracted with DCM (2×150 mL). The organic extracts were dried (phase separator) and concentrated in vacuo. The crude was dissolved in EtOH (10 mL) and conc HCl (10 mL) was added and stirred at 120° C. for 16 h. The volatiles were evaporated in vacuo. The solid was partitioned between DCM (so mL) and 2 M NaOH (20 mL. The organics were separated and the aqueous layers were extracted with DCM (2×20 mL). The organic extracts were combined, dried (MgSO₄) and concentrated in vacuo to afford the title compound (336 mg, 68%) as a orange solid.

LCMS m/z 193.1 (M+H)⁺ (ES⁺).

¹H NMR (DMSO-d₆) δ 7.21 (s, 1H), 6.62 (s, 2H), 3.16 (t, J=7.5 Hz, 2H), 2.76 (t, J=7.6 Hz, 2H), 2.16 (s, 3H), 1.97 (p, J=7.5 Hz, 2H).

### Step D: 5-bromo-6-methyl-4-nitro-2,3-dihydro-1H-indene

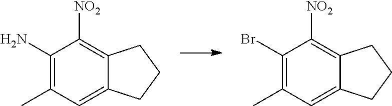

A solution of 6-methyl-4-nitro-2,3-dihydro-1H-inden-5-amine (336 mg, 1.748 mmol) and isopentyl nitrite (0.26 mL, 1.94 mmol) in MeCN (35 mL) was heated to 55° C. whereupon CuBr₂ (312 mg, 1.398 mmol) was added. The mixture was heated at 55° C. for min and 30 min at 70° C. After cooling to RT, 1 M HCl (33 mL) was added and the product was extracted with DCM (3×50 mL). The organics were concentrated in vacuo. The crude product was purified by chromatography on silica gel (24 g column, 0-50% EtOAc/isohexane) to afford the title compound (218 mg, 46%) as a white solid.

¹H NMR (DMSO-d₆) δ 7.50 (s, 1H), 2.95-2.88 (m, 4H), 2.41 (s, 3H), 2.09 (p, J=7.5 Hz, 2H).

### Step E: 2-methoxy-4-(6-methyl-4-nitro-2,3-dihydro-1H-inden-5-yl)pyridine

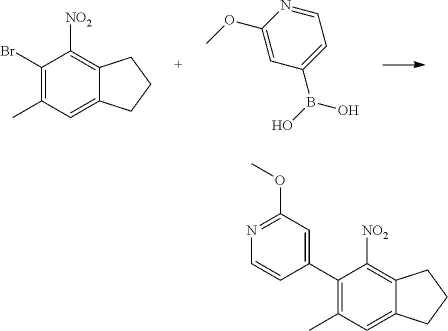 [SMILES: Cc1cc2c(c([N+](=O)[O-])c1Br)CCC2 ; COc1cc(B(O)O)ccn1 ; COc1cc(-c2c(C)cc3c(c2[N+](=O)[O-])CCC3)ccn1]

Prepared according to the general procedure of 5-(pyridin-4-yl)-2,3-dihydro-1H-inden-4-amine (Intermediate C4) from 5-bromo-6-methyl-4-nitro-2,3-dihydro-1H-indene and (2-methoxypyridin-4-yl)boronic acid to afford the title compound (186 mg, 63%). LCMS m/z 285.0 (M+H)⁺ (ES⁺).

¹H NMR (DMSO-d₆) δ 8.24 (d, J=5.2 Hz, 1H), 7.50 (s, 1H), 6.88-6.81 (m, 1H), 6.67 (d, J=2.0 Hz, 1H), 3.89 (s, 3H), 3.03-2.92 (m, 4H), 2.18-2.03 (m, 5H).

### Step F: s-(2-methoxypyridin-4-yl)-6-methyl-2,3-dihydro-1H-inden-4-amine

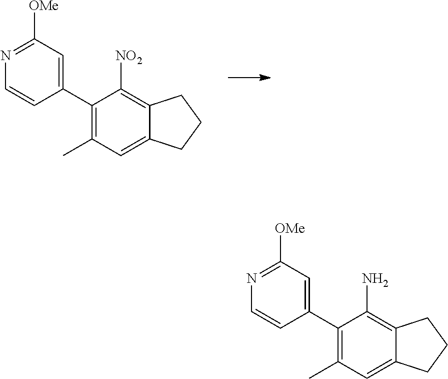 [SMILES: COc1cc(-c2c(C)cc3c(c2[N+](=O)[O-])CCC3)ccn1 ; COc1cc(-c2c(C)cc3c(c2N)CCC3)ccn1]

A mixture of 2-methoxy-4-(6-methyl-4-nitro-2,3-dihydro-1H-inden-5-yl)pyridine (186 mg, 0.536 mmol) and 5% Pd—C(Type 87L, 58-5% moisture, 55 mg, 10.72 μmol) in EtOH (2 mL) was hydrogenated at 1 bar for 6 h. The mixture was filtered through Celite and evaporated to afford the title compound (120 mg, 77%).

LCMS m/z 255.1 (M+H)⁺ (ES⁺).

¹H NMR (DMSO-d₆) δ 8.24 (d, J=5.2 Hz, 1H), 6.77 (dd, J=5.2, 1.5 Hz, 1H), 6.58 (s, 1H), 6.45 (s, 1H), 4.16 (s, 2H), 3.89 (s, 3H), 2.78 (t, J=7.5 Hz, 2H), 2.64 (t, J=7.4 Hz, 2H), 1.99 (p, J=7.4 Hz, 2H), 1.88 (s, 3H).

### Intermediate C21: 5-(2-(methoxy-d₃)pyridin-4-yl)-2,3-dihydro-1H-inden-4-amine

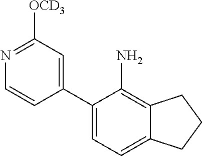

### Step A: 4-bromo-2-(methoxy-d₃)pyridine

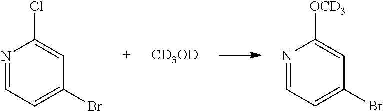

A solution of methanol-d4 (520 mg, 14.42 mmol) in THF (5 mL) was added slowly to a suspension of NaH (60% in mineral oil, 0.577 g, 14.42 mmol) in THF (15 mL) and stirred for 10 min at RT. The reaction was cooled to 5° C. and a solution of 4-bromo-2-chloropyridine (1 mL, 9.01 mmol) in THF (5 mL) was added over 5 min. The reaction was warmed to RT and stirred for 6 days, diluted with THF (20 mL), cooled to 5° C. and quenched with water (15 mL). The mixture was extracted with EtOAc (2×20 mL). The product was purified by chromatography on silica gel (12 g column, 0-10% EtOAc/isohexane) to afford the title compound (390 mg, 21%) as a colourless oil.

LCMS m/z 191.0/193.0 (M+H)⁺ (ES⁺).

¹H NMR (CDCl₃) δ 8.01 (d, J=5.5 Hz, 1H), 7.06-7.02 (m, 1H), 6.97 (d, J=1.7 Hz, 1H).

### Step B: 5-(4,4,5,5-tetramethyl-1,3,2-dioxaborolan-2-yl)-2,3-dihydro-1H-inden-4-amine

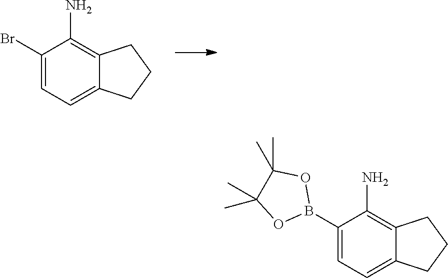 [SMILES: Nc1c(Br)ccc2c1CCC2 ; CC1(C)OB(c2ccc3c(c2N)CCC3)OC1(C)C]

PdCl₂(dppf).DCM (0.770 g, 0.943 mmol) was added was added to a mixture of 5-bromo-2,3-dihydro-1H-inden-4-amine (Intermediate R4, Step E) (2 g, 9.43 mmol), B₂Pin₂ (2.51 g, 9.90 mmol) and KOAc (3.24 g, 33.0 mmol) in 1,4-dioxane (14 mL) under N₂. The reaction was stirred at 80° C. for 2 h, diluted with water (10 mL) and extracted with EtOAc (2×20 mL). The organics were washed with water (20 mL), brine (20 mL), dried and concentrated in vacuo. The product was purified by chromatography on silica gel (120 g column, 0-10% EtOAc/isohexane) to afford the title compound (844 mg, 34%) as a white solid.

LCMS m/z 260.2 (M+H)⁺ (ES⁺).

¹H NMR (DMSO-d₆) δ 7.22 (d, J=7.4 Hz, 1H), 6.44 (d, J=7.6 Hz, 1H), 5.21 (s, 2H), 2.79 (t, J=7.6 Hz, 2H), 2.61 (t, J=7.4 Hz, 2H), 1.97 (p, J=7.5 Hz, 2H), 1.29 (s, 12H).

### Step C: 5-(2-(methoxy-d₃)pyridin-4-yl)-2,3-dihydro-1H-inden-4-amine

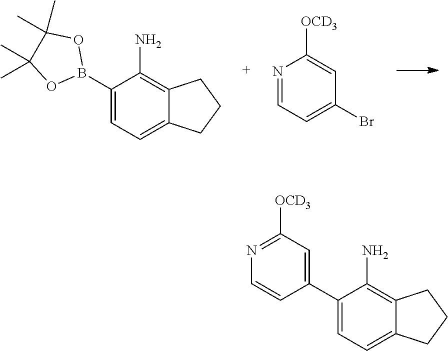

Prepared according to the general procedure of 5-(pyridin-4-yl)-2,3-dihydro-1H-inden-4-amine (Intermediate C4) from 5-(4,4,5,5-tetramethyl-1,3,2-dioxaborolan-2-yl)-2,3-dihydro-1H-inden-4-amine and 4-bromo-2-(methoxy-d₃)pyridine to afford the title compound (250 mg, 54%) as a off-white sold.

LCMS m/z 244.2 (M+H)⁺ (ES⁺).

¹H NMR (DMSO-d₆) δ 8.18 (d, J=5.3 Hz, 1H), 7.01 (d, J=5.2 Hz, 1H), 6.85 (d, J=7.6 Hz, 1H), 6.79 (s, 1H), 6.58 (d, J=7.8 Hz, 1H), 4.65 (s, 2H), 2.83 (t, J=7.5 Hz, 2H), 2.70 (t, J=7.4 Hz, 2H), 2.02 (p, J=7.5 Hz, 2H).

### Intermediate C22: 5-(2-(methoxy-d₃)pyridin-4-yl)-2,3-dihydrobenzofuran-4-amine

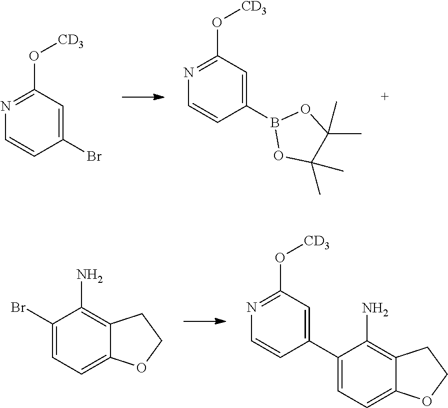 [SMILES: [2H]C([2H])([2H])Oc1cc(Br)ccn1 ; [2H]C([2H])([2H])Oc1cc(B2OC(C)(C)C(C)(C)O2)ccn1 ; Nc1c(Br)ccc2c1CCO2 ; [2H]C([2H])([2H])Oc1cc(-c2ccc3c(c2N)CCO3)ccn1]

To a solution of 4-bromo-2-(methoxy-d₃)pyridine (Intermediate C21, Step A) (1.076 g, 5.63 mmol) in dioxane (25 mL) was added B₂Pin₂ (1.573 g, 6.20 mmol) followed by KOAc (2.211 g, 22.53 mmol). The reaction mixture was heated to 6° C. and degassed with N₂. PdCl₂(dppf).DCM (0.23 g, 0.282 mmol) was added and the reaction was heated to 100° C. for 2 h. After cooling, 5-bromo-2,3-dihydrobenzofuran-4-amine (Intermediate C5, Step B) (1.326 g, 6.20 mmol) was added followed by a solution of K₂CO₃ (3.11 g, 22.53 mmol) in water (3 mL) and the reaction was heated at 100° C. for 1 h, diluted with EtOAc (150 mL) and washed with brine (100 mL). The organics were dried (MgSO₄) and concentrated in vacuo. The crude product was purified by chromatography on silica gel (24 g column, 0-60% EtOAc/isohexane) to afford a solid which was sonicated with iso-hexanes (5 mL). The solid was collected by filtration to afford the title compound (0.662 g, 47%) as a slightly tanned solid.

LCMS m/z 246.2 (M+H)⁺ (ES⁺).

¹H NMR (DMSO-d₆) δ 8.15 (d, J=5.3 Hz, 1H), 7.02-6.93 (m, 1H), 6.83 (d, J=8.1 Hz, 1H), 6.76 (s, 1H), 6.13 (d, J=8.1 Hz, 1H), 4.90 (s, 2H), 4.53 (t, J=8.7 Hz, 2H), 3.00 (t, J=8.7 Hz, 2H).

### Intermediate C23:5-(2-methylpyridin-4-yl)-2,3-dihydrobenzofuran-4-amine

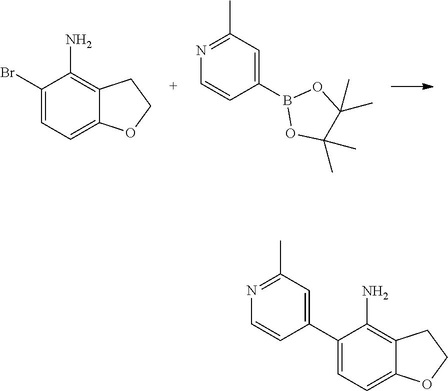 [SMILES: Nc1c(Br)ccc2c1CCO2 ; Cc1cc(B2OC(C)(C)C(C)(C)O2)ccn1 ; Cc1cc(-c2ccc3c(c2N)CCO3)ccn1]

A solution of K₂CO₃ (1356 mg, 9.81 mmol) in water (2 mL) was added to 5-bromo-2,3-dihydrobenzofuran-4-amine (Intermediate C5, Step B) (700 mg, 3.27 mmol) in dioxane (8 mL) followed by 2-methyl-4-(4,4,5,5-tetramethyl-1,3,2-dioxaborolan-2-yl)pyridine (716 mg, 3.27 mmol). The mixture was degassed with N₂ and Pd(dppf)Cl₂.DCM (134 mg, 0.164 mmol) was added. The reaction was then heated to 80° C. for 20 h, cooled to RT and partitioned between DCM (30 mL) and water (20 mL). The organic phase was dried (phase separator) and concentrated in vacuo. The product was purified by chromatography on silica gel (24 g column, 0-100% EtOAc/isohexane) to afford the title compound (492 mg, 63%) as a tan solid.

LCMS m/z 227.2 (M+H)⁺ (ES⁺).

¹H NMR (DMSO-d₆) 6.8.41 (d, J=5.2 Hz, 1H), 7.24 (s, 1H), 7.18 (dd, J=5.2, 1.7 Hz, 1H), 6.82 (d, J=8.1 Hz, 1H), 6.14 (d, J=8.1 Hz, 1H), 4.90 (s, 2H), 4.54 (t, J=8.7 Hz, 2H), 3.01 (t, J=8.7 Hz, 2H), 2.48 (s, 3H).

### Intermediate C24: 5-(2-(difluoromethoxy)pyridin-4-yl)-2,3-dihydrobenzofuran-4-amine

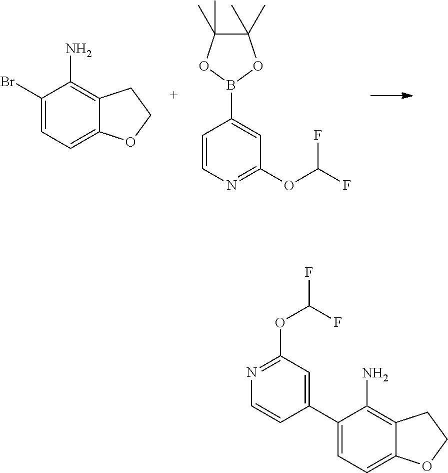 [SMILES: Nc1c(Br)ccc2c1CCO2 ; CC1(C)OB(c2ccnc(OC(F)F)c2)OC1(C)C ; Nc1c(-c2ccnc(OC(F)F)c2)ccc2c1CCO2]

Prepared according to the general procedure of 5-(2-methylpyridin-4-yl)-2,3-dihydro-benzofuran-4-amine (Intermediate C23) from 5-bromo-2,3-dihydrobenzofuran-4-amine (Intermediate C5, Step B) and 2-(difluoromethoxy)-4-(4,4,5,5-tetramethyl-1,3,2-dioxaborolan-2-yl)pyridine (Intermediate C8, Step B) to afford the title compound (0.76 g, 53%) as an orange solid.

LCMS m/z 279.0 (M+H)⁺ (ES⁺).

¹H NMR (DMSO-d₆) δ 8.25-8.19 (m, 1H), 7.73 (t, J=73.1 Hz, 1H), 7.28 (dd, J=5.3, 1.5 Hz, 1H), 7.03 (d, J=1.4 Hz, 1H), 6.88 (d, J=8.2 Hz, 1H), 6.16 (d, J=8.2 Hz, 1H), 5.04 (s, 2H), 4.55 (t, J=8.7 Hz, 2H), 3.01 (t, J=8.7 Hz, 2H).

### Intermediate C25: 5-(pyridin-4-yl)-2,3-dihydrobenzofuran-4-amine

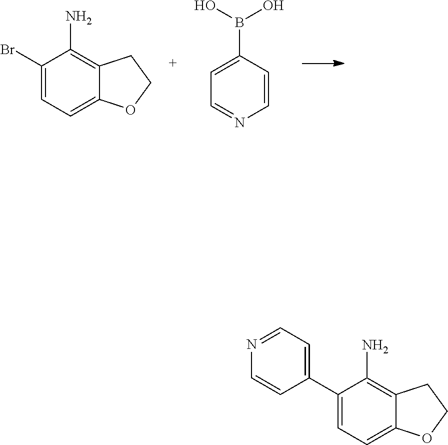 [SMILES: Nc1c(Br)ccc2c1CCO2 ; OB(O)c1ccncc1 ; Nc1c(-c2ccncc2)ccc2c1CCO2]

Prepared according to the general procedure of 5-(2-methylpyridin-4-yl)-2,3-dihydro-benzofuran-4-amine (Intermediate C23) from 5-bromo-2,3-dihydrobenzofuran-4-amine (Intermediate C5, Step B) and pyridin-4-ylboronic acid hydrate to afford the title compound (180 mg, 51%) as a tan solid.

LCMS m/z 213.1 (M+H)⁺ (ES⁺).

¹H NMR (DMSO-d₆) δ. 8.55 (dd, J=4.4, 1.7 Hz, 2H), 7.39 (dd, J=4.5, 1.7 Hz, 2H), 6.84 (d, J=8.1 Hz, 1H), 6.16 (d, J=8.1 Hz, 1H), 4.94 (s, 2H), 4.54 (t, J=8.7 Hz, 2H), 3.01 (t, J=8.7 Hz, 2H).

### Intermediate C26: 1-isopropyl-4-(pyridin-4-yl)-1H-pyrazol-5-amine

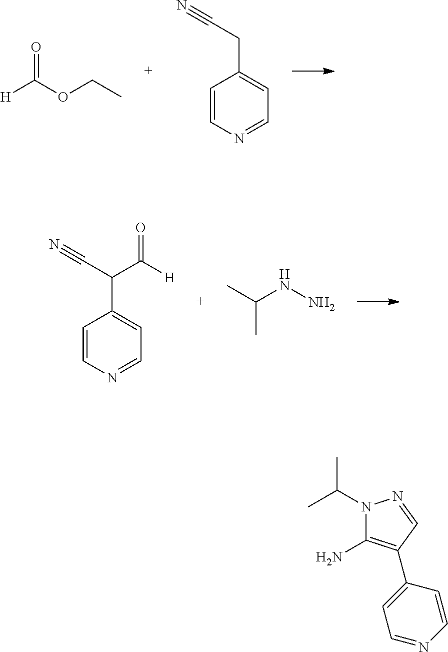

2-(Pyridin-4-yl)acetonitrile hydrochloride (1.00 g, 6.47 mmol) was added to a solution of EtONa (21% in EtOH, 5.55 mL, 14.88 mmol). Ethyl formate (0.624 mL, 7.76 mmol) was added and the reaction mixture was heated to reflux for 2 h. The solvent was evaporated in vacuo. Water was added (10 mL) and the pH was adjusted to 4 by adding AcOH. EtOH (30 mL) was added, followed by isopropylhydrazine (959 mg, 12.94 mmol) and the reaction was heated to reflux for 16 h. The mixture was evaporated and sat aq NaHCO₃ (20 mL) was added and extracted with EtOAc (2×50 mL). The organic extract was dried (MgSO₄) and evaporated. The crude product was purified by chromatography on silica gel (12 g column, 0-10% (0.7 M ammonia/MeOH)/DCM) to afford the title compound (397 mg, 29%) as an orange gum.

LCMS m/z 203.4 (M+H)⁺ (ES⁺).

¹H NMR (DMSO-d₆) δ 8.42-8.36 (m, 2H), 7.66 (s, 1H), 7.45-7.42 (m, 2H), 5.67 (s, 2H), 4.55 (sept, J=6.5 Hz, 1H), 1.32 (d, J=6.5 Hz, 6H).

### Intermediate C27: 4-isopropyl-1-(pyridin-4-yl)-1H-pyrazol-5-amine

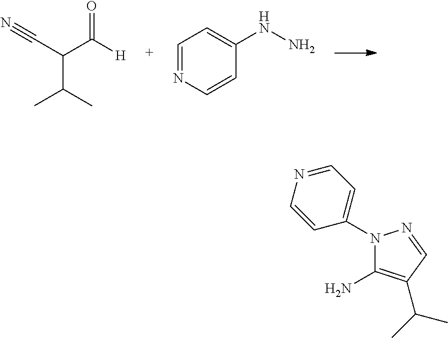

4-Hydrazinylpyridine (1.00 g, 9.16 mmol) was added to a solution of 2-formyl-3-methylbutanenitrile (1.14 g, 10.26 mmol) in EtOH (60 mL) and the reaction was heated to reflux for 16 h. The mixture was concentrated in vacuo and sat aq NaHCO₃ (20 mL) was added. The product was extracted with EtOAc (2×50 mL). The organics were dried (MgSO₄) and evaporated. The product was purified by chromatography on silica gel (12 g column, 0-10% (0.7 M ammonia/MeOH)/DCM) to afford the title compound (586 mg, 22%) as an orange solid.

LCMS m/z 203.1 (M+H)⁺ (ES⁺).

¹H NMR (DMSO-d₆) δ 8.63-8.52 (m, 2H), 7.77-7.71 (m, 2H), 7.40 (s, 1H), 5.25 (s, 2H), 2.83 (sept, J=6.8 Hz, 1H), 1.14 (d, J=6.9 Hz, 6H).

### Intermediate C28: 5-(2-(benzyloxy)pyridin-4-yl)-2,3-dihydro-1H-inden-4-amine

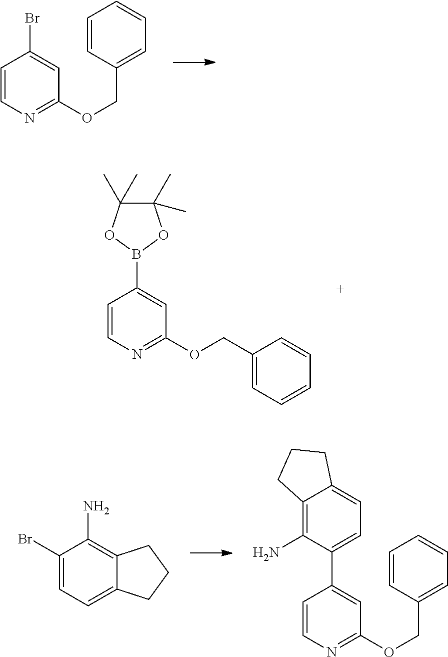

Prepared according to the general procedure of 5-(2-(methoxy-d₃)pyridin-4-yl)-2,3-dihydrobenzofuran-4-amine (Intermediate C22) from 2-(benzyloxy)-4-bromo-pyridine and 5-bromo-2,3-dihydro-1H-inden-4-amine to afford the title compound (415 mg, 77%) as a white solid.

LCMS m/z 317.2 (M+H)⁺ (ES⁺).

¹H NMR (DMSO-d₆) δ 8.19 (d, J=5.3 Hz, 1H), 7.49 (d, J=7.1 Hz, 2H), 7.39 (t, J=7.2 Hz, 2H), 7.33 (t, J=7.3 Hz, 1H), 7.04 (dd, J=5.3, 1.5 Hz, 1H), 6.91-6.81 (m, 2H), 6.58 (d, J=7.6 Hz, 1H), 5.39 (s, 2H), 4.68 (s, 2H), 2.83 (t, J=7.4 Hz, 2H), 2.71 (t, J=7.4 Hz, 2H), 2.02 (p, J=7.4 Hz, 2H).

### Intermediate C29:5-(2-((1-methylpiperidin-4-yl)oxy)pyridin-4-yl)-2,3-dihydro-1H-inden-4-amine

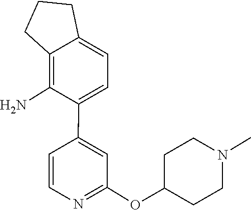 [SMILES: CN1CCC(Oc2cc(-c3ccc4c(c3N)CCC4)ccn2)CC1]

### Step A: 4-bromo-2-((1-methylpiperidin-4-yl)oxy)pyridine

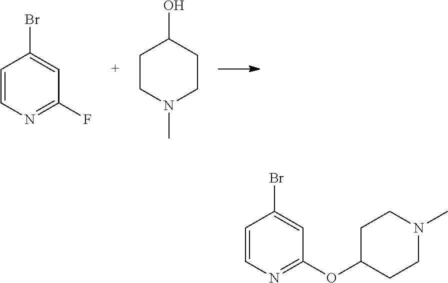

NaO^{t}Bu (2 M in THF, 3.72 mL, 7.44 mmol) was added to 1-methylpiperidin-4-ol(1.168 g, 10.15 mmol) in THF (3 mL) and the reaction was stirred at RT for 1 h and cooled to 0° C. 4-bromo-2-fluoropyridine (0.695 mL, 6.76 mmol) was added and the reaction was stirred at RT for 3 h and partitioned between MTBE (50 mL) and water (30 mL). The organic layer was washed with water (30 mL), dried (phase separator) and concentrated in vacuo. The crude product was purified by chromatography on silica gel (24 g column, 0-10% (0.7 M ammonia/MeOH)/DCM) to afford the title compound (1.14 g, 60%) as a clear yellow oil.

LCMS m/z 271.1/273.1 (M+H)⁺ (ES⁺).

¹H NMR (DMSO-d₆) δ 8.06 (d, J=5.5 Hz, 1H), 7.20 (dd, J=5.5, 1.7 Hz, 1H), 7.08 (d, J=1.6 Hz, 1H), 4.97 (m, 1H), 2.70-2.56 (m, 2H), 2.20-2.07 (m, 5H), 1.99-1.88 (m, 2H), 1.71-1.59 (m, 2H).

### Step B: 5-(2-((1-methylpiperidin-4-yl)oxy)pyridin-4-yl)-2,3-dihydro-1H-inden-4-amine

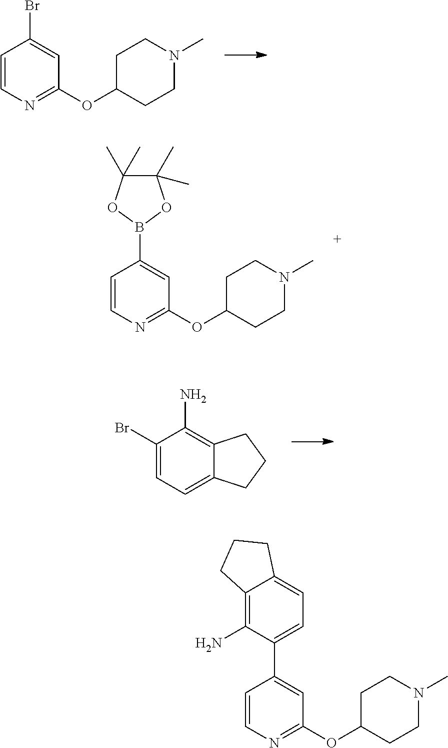

Prepared according to the general procedure of 5-(2-(methoxy-d₃)pyridin-4-yl)-2,3-dihydrobenzofuran-4-amine (Intermediate C22) from 4-bromo-2-((1-methyl-piperidin-4-yl)oxy)pyridine and 5-bromo-2,3-dihydro-1H-inden-4-amine (Intermediate R4, Step E) to afford the title compound (404 mg, 75%) as a brown gum.

LCMS m/z 324.3 (M+H)⁺ (ES⁺).

¹H NMR (DMSO-d₆) δ 8.16 (d, J=5.3 Hz, 1H), 6.98 (dd, J=5.3, 1.5 Hz, 1H), 6.85 (d, J=7.6 Hz, 1H), 6.72 (s, 1H), 6.57 (d, J=7.6 Hz, 1H), 5.00 (m, 1H), 4.64 (s, 2H), 2.83 (t, J=7.5 Hz, 2H), 2.75-2.60 (m, 4H), 2.24-2.08 (m, 5H), 2.08-1.93 (m, 4H), 1.75-1.60 (m, 2H).

### Intermediate C30: 5-(2-((tetrahydro-2H-pyran-3-yl)oxy)pyridin-4-yl)-2,3-dihydro-1H-inden-4-amine

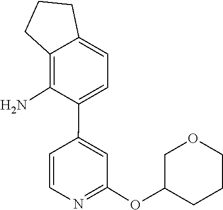 [SMILES: Nc1c(-c2ccnc(OC3CCCOC3)c2)ccc2c1CCC2]

### Step A: 4-bromo-2-((tetrahydro-2H-pyran-3-yl)oxy)pyridine

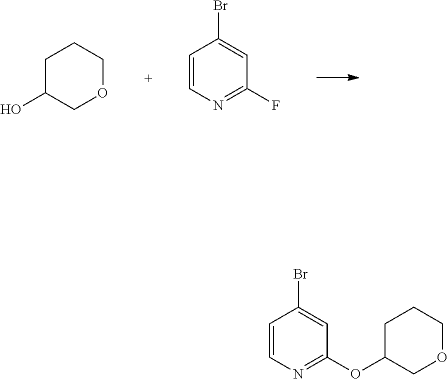 [SMILES: OC1CCCOC1 ; Fc1cc(Br)ccn1 ; Brc1ccnc(OC2CCCOC2)c1]

Prepared according to the general procedure of 4-bromo-2-((1-methylpiperidin-4-yl)-oxy)pyridine (Intermediate C29, Step A) from tetrahydro-2H-pyran-3-ol and 4-bromo-2-fluoropyridine to afford the title compound (466.3 mg, 62%) as a clear colourless oil.

LCMS m/z 258.0/260.0 (M+H)⁺ (ES⁺).

¹H NMR (DMSO-d₆) δ 8.05 (d, J=5.5 Hz, 1H), 7.20 (dd, J=5.5, 1.7 Hz, 1H), 7.09 (d, J=1.6 Hz, 1H), 5.02-4.95 (m, 1H), 3.86-3.79 (m, 1H), 3.65-3.58 (m, 1H), 3.56-3.46 (m, 2H), 2.05-1.97 (m, 1H), 1.82-1.68 (m, 2H), 1.57-1.48 (m, 1H).

### Step B: 5-(2-((tetrahydro-2H-pyran-3-yl)oxy)pyridin-4-yl)-2,3-dihydro-1H-inden-4-amine

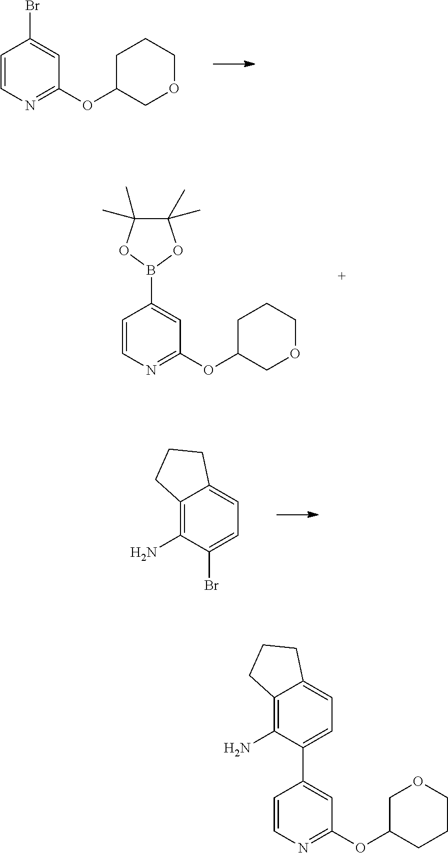 [SMILES: Brc1ccnc(OC2CCCOC2)c1 ; CC1(C)OB(c2ccnc(OC3CCCOC3)c2)OC1(C)C ; Nc1c(Br)ccc2c1CCC2 ; Nc1c(-c2ccnc(OC3CCCOC3)c2)ccc2c1CCC2]

Prepared according to the general procedure of 5-(2-(methoxy-d₃)pyridin-4-yl)-2,3-dihydrobenzofuran-4-amine (Intermediate C22) from 4-bromo-2-((tetrahydro-2H-pyran-3-yl)oxy)pyridine and 5-bromo-2,3-dihydro-1H-inden-4-amine (Intermediate R⁴, Step E) to afford the title compound (0.393 g, 79%) as a pale yellow oil.

LCMS m/z 311.0 (M+H)⁺ (ES⁺); 308.8 (M−H)⁻ (ES⁻).

¹H NMR (DMSO-d₆) δ 8.16 (dd, J=5.2, 0.7 Hz, 1H), 7.01 (dd, J=5.3, 1.5 Hz, 1H), 6.85 (d, J=7.6 Hz, 1H), 6.76-6.74 (m, 1H), 6.58 (d, J=7.6 Hz, 1H), 5.08-4.98 (m, 1H), 4.66 (s, 2H), 3.92-3.87 (m, 1H), 3.69-3.61 (m, 1H), 3.56-3.48 (m, 2H), 2.83 (t, J=7.5 Hz, 2H), 2.70 (t, J=7.3 Hz, 2H), 2.11-2.00 (m, 3H), 1.85-1.71 (m, 2H), 1.61-1.51 (m, 1H).

### Intermediate C31: 5-(2-((1-methoxypropan-2-yl)oxy)pyridin-4-yl)-2,3-dihydro-1H-inden-4-amine

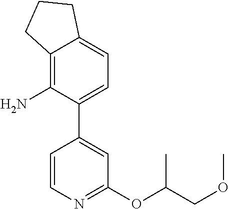 [SMILES: COCC(C)Oc1cc(-c2ccc3c(c2N)CCC3)ccn1]

### Step A: 4-bromo-2-((1-methoxypropan-2-yl)oxy)pyridine

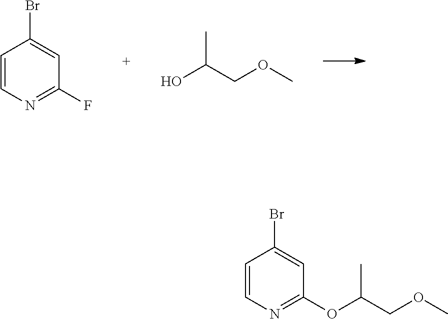

Prepared according to the general procedure of 4-bromo-2-((1-methylpiperidin-4-yl)-oxy)pyridine (Intermediate C29, Step A) from 1-methoxypropan-2-ol and 4-bromo-2-fluoropyridine to afford the title compound (1.26 g, 88%) as a clear colourless oil. LCMS m/z 246.0/248.0 (M+H)⁺ (ES⁺).

¹H NMR (DMSO-d₆) δ 8.06 (dd, J=5.5, 0.4 Hz, 1H), 7.19 (dd, J=5.5, 1.7 Hz, 1H), 7.06 (d, J=1.5 Hz, 1H), 5.36-5.27 (m, 1H), 3.53-3.42 (m, 2H), 3.27 (s, 3H), 1.23 (d, J=6.4 Hz, 3H).

### Step B: 5-(2-((1-methoxypropan-2-yl)oxy)pyridin-4-yl)-2,3-dihydro-1H-inden-4-amine

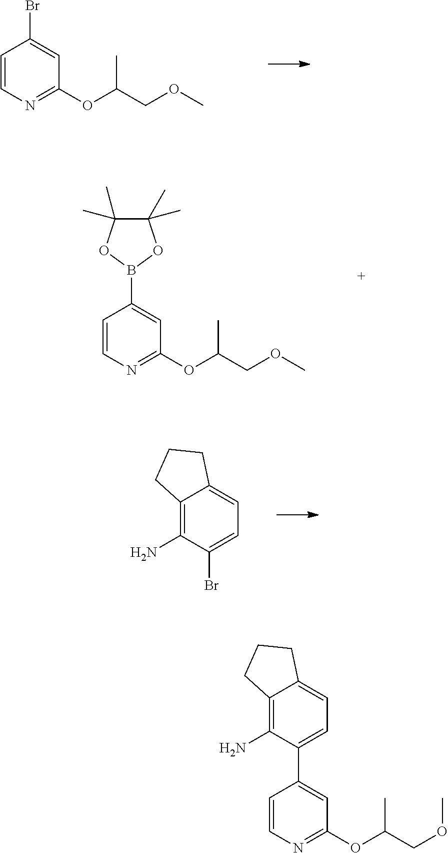

Prepared according to the general procedure of 5-(2-(methoxy-d₃)pyridin-4-yl)-2,3-dihydrobenzofuran-4-amine (Intermediate C22) from 4-bromo-2-((1-methoxy-propan-2-yl)oxy)pyridine and 5-bromo-2,3-dihydro-1H-inden-4-amine (Intermediate R4, Step E) to afford the title compound (0.349 g, 76%) as a pale yellow oil.

LCMS m/z 299.0 (M+H)⁺ (ES⁺).

¹H NMR (DMSO-d₆) δ 8.16 (dd, J=5.3, 0.7 Hz, 1H), 6.99 (dd, J=5.3, 1.5 Hz, 1H), 6.85 (d, J=7.6 Hz, 1H), 6.72-6.71 (m, 1H), 6.58 (d, J=7.6 Hz, 1H), 5.39-5.31 (m, 1H), 4.65 (s, 2H), 3.56 (dd, J=10.4, 6.1 Hz, 1H), 3.47 (dd, J=10.3, 4.2 Hz, 1H), 3.30 (s, 3H), 2.83 (t, J=7.5 Hz, 2H), 2.70 (t, J=7.3 Hz, 2H), 2.03 (p, J=7.4 Hz, 2H), 1.27 (d, J=6.4 Hz, 3H).

### Intermediate C32: 2-((4-(4-amino-2,3-dihydro-1H-inden-5-yl)pyridin-2-yl)oxy)ethanol

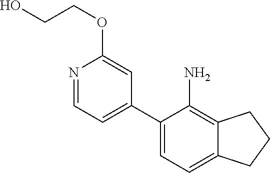 [SMILES: Nc1c(-c2ccnc(OCCO)c2)ccc2c1CCC2]

### Step A: 2-(2-(benzyloxy)ethoxy)-4-bromopyridine

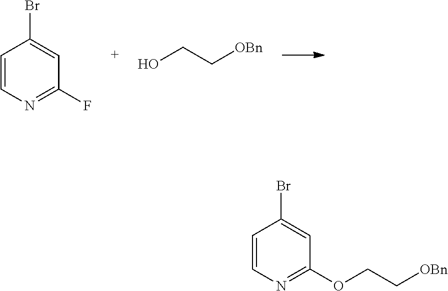 [SMILES: Fc1cc(Br)ccn1 ; OCCOCc1ccccc1 ; Brc1ccnc(OCCOCc2ccccc2)c1]

Prepared according to the general procedure of 4-bromo-2-((1-methylpiperidin-4-yl)-oxy)pyridine (Intermediate C29, Step A) from 2-(benzyloxy)ethanol and 4-bromo-2-fluoropyridine to afford the title compound (1.6 g, 90%) as a colourless oil.

LCMS m/z 307.9/309.9 (M+H)⁺ (ES⁺).

¹H NMR (CDCl₃) δ 7.98 (d, J=5.5 Hz, 1H), 7.40-7.29 (m, 5H), 7.06-7.02 (m, 2H), 4.64 (s, 2H), 4.53-4.51 (m, 2H), 3.85-3.82 (m, 2H).

### Step B: 5-(2-(2-(benzyloxy)ethoxy)pyridin-4-yl)-2,3-dihydro-1H-inden-4-amine

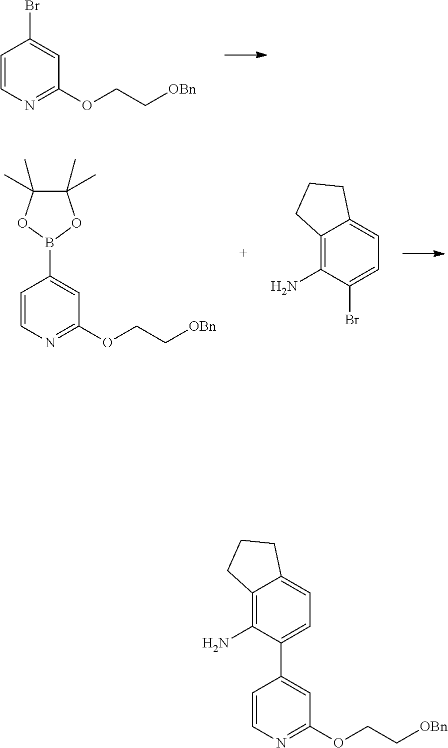 [SMILES: Brc1ccnc(OCCOCc2ccccc2)c1 ; CC1(C)OB(c2ccnc(OCCOCc3ccccc3)c2)OC1(C)C ; Nc1c(Br)ccc2c1CCC2 ; Nc1c(-c2ccnc(OCCOCc3ccccc3)c2)ccc2c1CCC2]

Prepared according to the general procedure of 5-(2-(methoxy-d₃)pyridin-4-yl)-2,3-dihydrobenzofuran-4-amine (Intermediate C22) from 2-(2-(benzyloxy)ethoxy)-4-bromopyridine and 5-bromo-2,3-dihydro-1H-inden-4-amine (Intermediate R4, Step E) to afford the title compound (1.215 g, 94%) as a clear orange oil.

LCMS m/z 361.1 (M+H)⁺ (ES⁺).

¹H NMR (DMSO-d₆) δ 8.17-8.14 (m, 1H), 7.37-7.31 (m, 4H), 7.30-7.25 (m, 1H), 7.01 (dd, J=5.3, 1.5 Hz, 1H), 6.85 (d, J=7.6 Hz, 1H), 6.79 (s, 1H), 6.58 (d, J=7.6 Hz, 1H), 4.65 (br s, 2H), 4.56 (s, 2H), 4.47-4.43 (m, 2H), 3.81-3.77 (m, 2H), 2.82 (t, J=7.5 Hz, 2H), 2.70 (t, J=7.4 Hz, 2H), 2.06-1.98 (m, 2H).

### Step C: 2-((4-(4-amino-2,3-dihydro-1H-inden-5-yl)pyridin-2-yl)oxy)ethanol

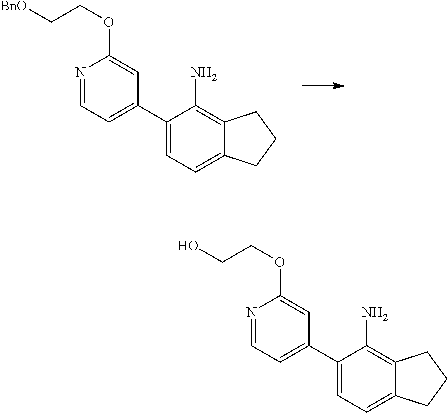 [SMILES: Nc1c(-c2ccnc(OCCOCc3ccccc3)c2)ccc2c1CCC2 ; Nc1c(-c2ccnc(OCCO)c2)ccc2c1CCC2]

A mixture of 5-(2-(2-(benzyloxy)ethoxy)pyridin-4-yl)-2,3-dihydro-1H-inden-4-amine (1.2 g, 3-33 mmol) and Pd/C (Type 87L, 58-5% water, 0.854 g, 0.166 mmol) in EtOH (5 mL) was hydrogenated at 3 bar at RT for 16 h. Pd(OH)₂ (0.8 g, 5-70 mmol) was added and the mixture was hydrogenated at 5 bar at RT for 4 h. The reaction was filtered over Celite with EtOAc (100 mL) and dried in vacuo to afford the title compound (556 mg, 54%).

LCMS m/z 271.0 (M+H)⁺ (ES⁺).

¹H NMR (DMSO-d₆) δ 8.16 (d, J=5.2 Hz, 1H), 7.00 (dd, J=5.3, 1.4 Hz, 1H), 6.85 (d, J=7.6 Hz, 1H), 6.78 (d, J=1.4 Hz, 1H), 6.58 (d, J=7.6 Hz, 1H), 4.83 (t, J=5.6 Hz, 1H), 4.66 (s, 2H), 4.30 (t, J=5.2 Hz, 2H), 3.48-3.42 (m, 2H), 2.83 (t, J=7.5 Hz, 2H), 2.70 (t, J=7.4 Hz, 2H), 2.02 (p, J=7.5 Hz, 2H).

### Intermediate C3: 2-isopropyl-6-(2-methoxypyridin-4-yl)phenol

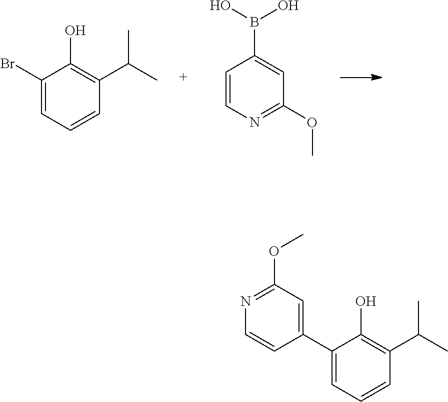 [SMILES: CC(C)c1cccc(Br)c1O ; COc1cc(B(O)O)ccn1 ; COc1cc(-c2cccc(C(C)C)c2O)ccn1]

A solution of 2-bromo-6-isopropylphenol (2.00 g, 9.30 mmol), (2-methoxypyridin-4-yl)boronic acid (1.564 g, 10.23 mmol), K₃CO₃ (4.24 g, 30.7 mmol) and Pd(dppf)Cl₂.DCM (1.519 g, 1.860 mmol) in dioxane (26 mL) and water (5.6 mL) was degassed with N₂. The reaction was heated at 85° C. for 4 h, cooled to RT and filtered through Celite, washing with EtOAc (100 mL). The filtrate was concentrated in vacuo and the product was purified by chromatography on silica gel (24 g column, 0-10% EtOAc/isohexane) to afford the title compound (1.76 g, 66%) as a yellow solid.

LCMS m/z 244.1 (M+H)⁺ (ES⁺).

¹H NMR (DMSO-d₆) δ 8.44 (s, 1H), 8.17 (d, J=5.3 Hz, 1H), 7.25-7.21 (m, 1H), 7.13-7.08 (m, 1H), 6.94 (t, J=7.6 Hz, 1H), 6.90 (s, 1H), 3.88 (s, 3H), 3.40-3.33 (m, 1H), 1.19 (d, J=6.8 Hz, 6H). One exchangeable proton not observed.

### Intermediate C34: 2-(2-methoxypyridin-4-yl)-3-methylaniline

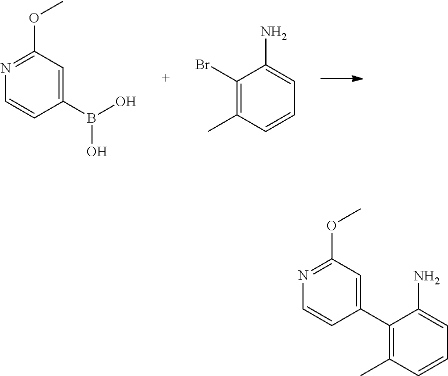

Prepared according to the general procedure of 5-(pyridin-4-yl)-2,3-dihydro-1H-inden-4-amine (Intermediate C4) from (2-methoxypyridin-4-yl)boronic acid and 2-bromo-3-methylaniline (1 g, 5.37 mmol) to afford the title compound (423 mg, 35%) as an orange solid.

LCMS m/z 215.2 (M+H)⁺ (ES⁺).

¹H NMR (DMSO-d₆) δ 8.24 (dd, J=5.1, 0.8 Hz, 1H), 6.96 (t, J=7.7 Hz, 1H), 6.79 (dd, J=5.2, 1.3 Hz, 1H), 6.61 (s, 1H), 6.60-6.57 (m, 1H), 6.52-6.46 (m, 1H), 4.46 (s, 2H), 3.89 (s, 3H), 1.91 (s, 3H).

### Intermediate D1:6-methyl-2-((5-(propylsulfonyl)-1-((2-(trimethylsilyl)ethoxy)-methyl)-1H-1,2,4-triazol-3-yl)sulfonyl)-2,6-diazaspiro[3-4]octane

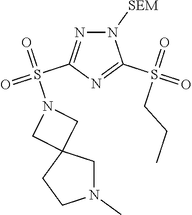 [SMILES: CCCS(=O)(=O)c1nc(S(=O)(=O)N2CC3(CCN(C)C3)C2)nn1COCC[Si](C)(C)C]

### Step A: 3-bromo-5-(propylthio)-1-((2-(trimethylsilyl)ethoxy)methyl)-1H-1,2,4-triazole

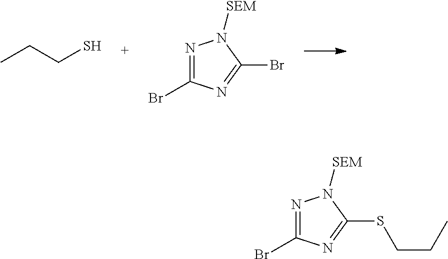

NaH (60% in mineral oil, 0.61 g, 16.78 mmol) was dissolved in DMF (50 mL) at 0° C. and propane-1-thiol (1.4 mL, 15.44 mmol) was added dropwise. The reaction was stirred at 0° C. for 20 min. A solution of 3,5-dibromo-1-((2-(trimethylsilyl)ethoxy)-methyl)-1H-1,2,4-triazole (5.5 g, 15.40 mmol) in DMF (20 mL) was added dropwise and the reaction was stirred for 16 h at RT, quenched with water (150 mL) and extracted with EtOAc (2×100 mL). The combined organic extracts were washed with brine (4×100 mL), dried (phase separator) and concentrated in vacuo to afford the title compound (5.55 g, 89%) as a light yellow oil.

¹H NMR (DMSO-d₆) δ 5.36 (s, 2H), 3.57 (t, J=8.2 Hz, 2H), 3.19 (t, J=7.1 Hz, 2H), 1.68 (sextet, J=7.1 Hz, 2H), 0.95 (t, J=7.4 Hz, 3H), 0.84 (t, J=7.9 Hz, 2H), −0.04 (s, 9H).

### Step B: methyl 3-((5-(propylthio)-1-((2-(trimethylsilyl)ethoxy)methyl)-1H-1,2,4-triazol-3-yl)thio)propanoate

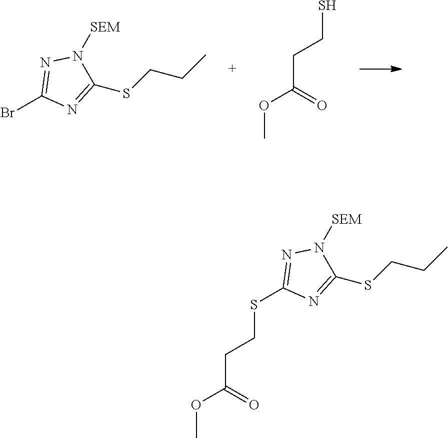

Prepared according to the general procedure of methyl 3-((5-((1,2,3,5,6,7-hexahydro-s-indacen-4-yl)amino)-1-((2-(trimethylsilyl)ethoxy)methyl)-1H-1,2,4-triazol-3-yl)thio)-propanoate (Intermediate B2, Step B) from 3-bromo-5-(propylthio)-1-((2-(trimethylsilyl)ethoxy)methyl)-1H-1,2,4-triazole and methyl 3-mercaptopropanoate to afford the title compound (2.35 g, 35%) as a yellow oil.

LCMS m/z 392.3 (M+H)⁺ (ES⁺).

¹H NMR (DMSO-d₆) δ 5.32 (s, 2H), 3.61 (s, 3H), 3.57 (t, J=8.0 Hz, 2H), 3.27 (t, J=7.0 Hz, 2H), 3.24 (t, J=7.1 Hz, 2H), 2.77 (t, J=7.0 Hz, 2H). 1.67 (sextet, J=7.2 Hz, 2H), 0.84 (t, J=7.3 Hz, 3H), 0.84 (t, J=8.0 Hz, 2H), −0.05 (s, 9H).

### Step C: methyl 3-((5-(propylsulfonyl)-1-((2-(trimethylsilyl)ethoxy)methyl)-1H-1,2,4-triazol-3-yl)sulfonyl)propanoate

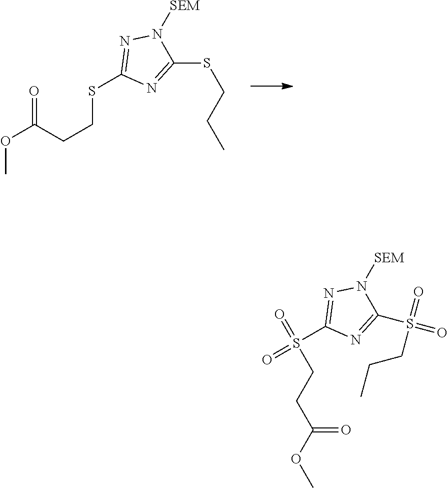 [SMILES: CCCSc1nc(SCCC(=O)OC)nn1COCC[Si](C)(C)C ; CCCS(=O)(=O)c1nc(S(=O)(=O)CCC(=O)OC)nn1COCC[Si](C)(C)C]

Prepared according to the general procedure of methyl 3-((5-((1,2,3,5,6,7-hexahydro-s-indacen-4-yl)amino)-1-((2-(trimethylsilyl)ethoxy)methyl)-1H-1,2,4-triazol-3-yl)sulfonyl)propanoate (Intermediate B2, Step C) from methyl 3-((5-(propylthio)-1-((2-(trimethylsilyl)ethoxy)methyl)-1H-1,2,4-triazol-3-yl)thio)propanoate to afford the title compound (2.72 g, 87%) as a colourless oil.

LCMS 454.3 (M−H)⁻ (ES⁻).

¹H NMR (DMSO-d₆) δ 5.87 (s, 2H), 3.79 (t, J=7.1 Hz, 2H), 3.72-3.66 (m, 4H), 3.59 (s, 3H), 2.82 (t, J=7.1 Hz, 2H), 1.75 (sextet, J=7.6 Hz, 2H), 0.99 (t, J=7.4 Hz, 3H), 0.88 (t, J=8.2 Hz, 2H), −0.03 (s, 9H).

### Step D: 6-methyl-2-((5-(propylsulfonyl)-1-((2-(trimethylsilyl)ethoxy)methyl)-1H-1,2,4-triazol-3-yl)sulfonyl)-2,6-diazaspiro[3.4]octane

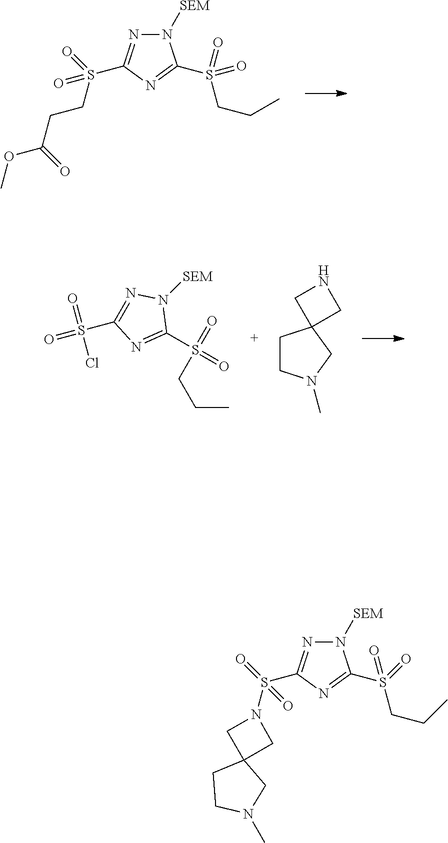

Methyl 3-((5-(propylsulfonyl)-1-((2-(trimethylsilyl)ethoxy)methyl)-1H-1,2,4-triazol-3-yl)sulfonyl)propanoate (500 mg, 1.097 mmol) was dissolved in THF (15 mL) and a solution of LiOH (26.3 mg, 1.097 mmol) in water (4 mL) was added. The reaction was stirred at RT for 30 min and concentrated in vacuo. The volatiles were removed azeotropically with heptane and the resulting residue was dissolved in DCM (12 mL) and cooled to 0° C. NCS (147 mg, 1.097 mmol) was added and the reaction was stirred for 45 min. A solution of 6-methyl-2,6-diazaspiro[3.4]octane (138 mg, 1.097 mmol) and Et₃N (0.229 mL, 1.646 mmol) in DCM (2 mL) was added and the reaction stirred at RT for 1 h. The reaction mixture was concentrated in vacuo and the crude was purified by chromatography on silica gel (24 g column, 0-10% (0.7 M ammonia/MeOH)/DCM) to afford the title compound (264.2 mg, 46%) as a colourless oil.

¹H NMR (DMSO-d₆) δ 5.92 (s, 2H), 3.97-3.90 (m, 4H), 3.79-3.68 (m, 4H), 2.49 (s, 2H), 2.40 (t, J=7.1 Hz, 2H), 2.18 (s, 3H), 1.89 (t, J=7.1 Hz, 2H), 1.81-1.72 (m, 2H), 1.01 (t, J=7.4 Hz, 3H), 0.91-0.87 (m, 2H), −0.03 (s, 9H).

### Intermediate D2: N,N-dimethyl-1-((5-(propylsulfonyl)-1-((2-(trimethylsilyl)ethoxy)-methyl)-1H-1,2,4-triazol-3-yl)sulfonyl)piperidin-4-amine

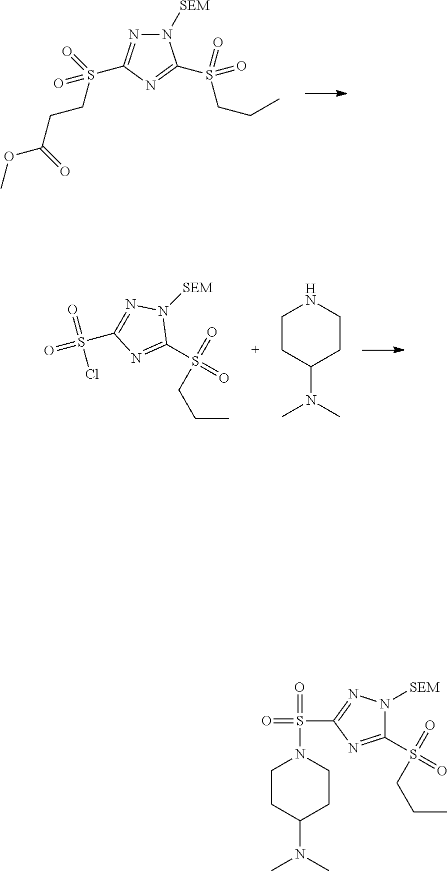

Prepared according to the general procedure of 6-methyl-2-((5-(propylsulfonyl)-1-((2-(trimethylsilyl)ethoxy)methyl)-1H-1,2,4-triazol-3-yl)sulfonyl)-2,6-diazaspiro[3.4]octane (Intermediate D1, Step D) from methyl 3-((5-(propylsulfonyl)-1-((2-(trimethylsilyl)ethoxy)methyl)-1H-1,2,4-triazol-3-yl)sulfonyl)-propanoate (Intermediate D1, Step C) and N,N-dimethylpiperidin-4-amine to afford the title compound (25 mg, 14%) as a clear colourless oil.

LCMS m/z 496.4 (M+H)⁺ (ES⁺); 494.6 (M−H)⁻ (ES⁻).

¹H NMR (DMSO-d₆) δ 5.86 (s, 2H), 3.77-359 (m, 6H), 2.80 (t, J=12.0 Hz, 2H), 2.23-2.08 (m, 7H), 1.86-1.68 (m, 4H), 1.49-135 (m, 2H), 0.99 (t, J=7.4 Hz, 3H), 0.87 (t, J=8.1 Hz, 2H), −0.04 (s, 9H).

### Intermediate D3: (1-((5-((1,2,3,5,6,7-hexahydro-s-indacen-4-yl)amino)-1-((2-(trimethylsilyl)ethoxy)methyl)-1H-1,2,4-triazol-3-yl)sulfonyl)piperidin-3-yl)methanol

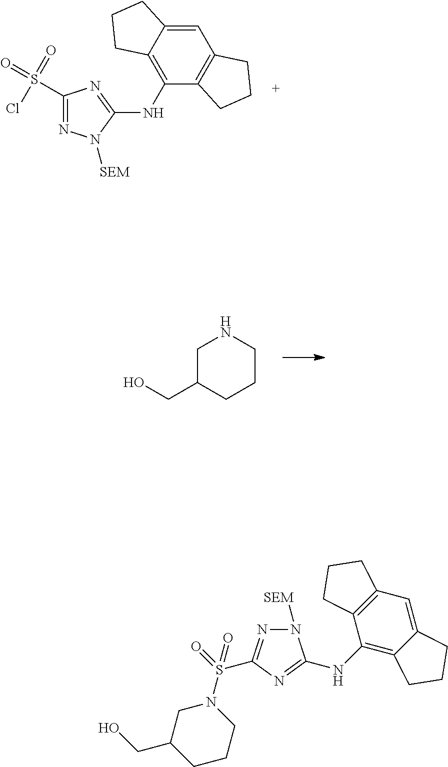 [SMILES: C[Si](C)(C)CCOCn1nc(S(=O)(=O)Cl)nc1Nc1c2c(cc3c1CCC3)CCC2 ; OCC1CCCNC1 ; C[Si](C)(C)CCOCn1nc(S(=O)(=O)N2CCCC(CO)C2)nc1Nc1c2c(cc3c1CCC3)CCC2]

To a stirred solution of piperidin-3-ylmethanol (0.184 g, 1.60 mmol) and Et₃N (0.222 mL, 1.60 mmol) in DCM (2 mL) was added 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) (0.50 g, 1.07 mmol) in DCM (10 mL). The reaction was stirred at RT for 90 min, concentrated in vacuo and purified by chromatography on silica gel (12 g column, 0-100% EtOAc/isohexane) to afford the title compound (0.370 g, 62%) as a yellow solid.

LCMS m/z 548.3 (M+H)⁺ (ES⁺).

¹H NMR (DMSO-d₆): δ 8.81 (s, 1H), 6.98 (s, 1H), 5.57-5.45 (m, 2H), 4.60 (t, J=5.2 Hz, 1H), 3.72-3.68 (m, 1H), 3.63-3.59 (m, 2H), 3.58-3.51 (m, 1H), 3.18-3.09 (m, 1H), 2.83 (t, J=7.4 Hz, 4H), 2.66 (app. q, J=7.0 Hz, 4H), 2.37-2.32 (m, 1H), 2.01-1.93 (m, 4H), 1.83-1.55 (m, 3H), 1.54-1.41 (m, 1H), 0.88 (dd, J=8.9, 7.3 Hz, 2H), −0.03 (s, 9H). Three protons obscured by water peak.

### Intermediate D4: (1-((5-((1,2,3,5,6,7-hexahydro-s-indacen-4-yl)amino)-1-((2-(trimethylsilyl)ethoxy)methyl)-1H-1,2,4-triazol-3-yl)sulfonyl)piperidin-4-yl)methanol

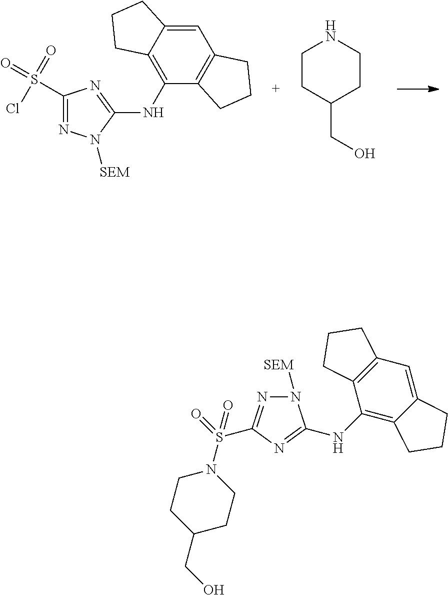 [SMILES: C[Si](C)(C)CCOCn1nc(S(=O)(=O)Cl)nc1Nc1c2c(cc3c1CCC3)CCC2 ; OCC1CCNCC1 ; C[Si](C)(C)CCOCn1nc(S(=O)(=O)N2CCC(CO)CC2)nc1Nc1c2c(cc3c1CCC3)CCC2]

Prepared according to the general procedure of (1-((5-((1,2,3,5,6,7-hexahydro-s-indacen-4-yl)amino)-1-((2-(trimethylsilyl)ethoxy)methyl)-1H-1,2,4-triazol-3-yl)-sulfonyl)piperidin-3-yl)methanol (Intermediate D3) from piperidin-4-ylmethanol and 5-((1,2,3,5,6,7-hexahydro-s-indacen-4-yl)amino)-1-((2-(trimethylsilyl)ethoxy)-methyl)-1H-1,2,4-triazole-3-sulfonyl chloride (Intermediate B4) to afford the title compound (0.332 g, 56%) as a pale yellow solid.

LCMS m/z 548.3 (M+H)⁺ (ES⁺).

¹H NMR (DMSO-d₆): δ 8.81 (s, 1H), 6.98 (s, 1H), 5.50 (s, 2H), 4.52 (t, J=5.2 Hz, 1H), 3.68-3.58 (m, 4H), 3.23 (t, J=5.8 Hz, 2H), 2.83 (t, J=7.4 Hz, 4H), 2.69-2.58 (m, 6H), 2.10-1.88 (m, 4H), 1.76-1.63 (m, 2H), 1.45-1.29 (m, 1H), 1.18-1.08 (m, 2H), 0.91-0.86 (m, 2H), −0.03 (s, 9H).

### Intermediate D5: (S)-(1-((5-((1,2,3,5,6,7-hexahydro-s-indacen-4-yl)amino)-1-((2-(trimethylsilyl)ethoxy)methyl)-1H-1,2,4-triazol-3-yl)sulfonyl)pyrrolidin-3-yl)methanol

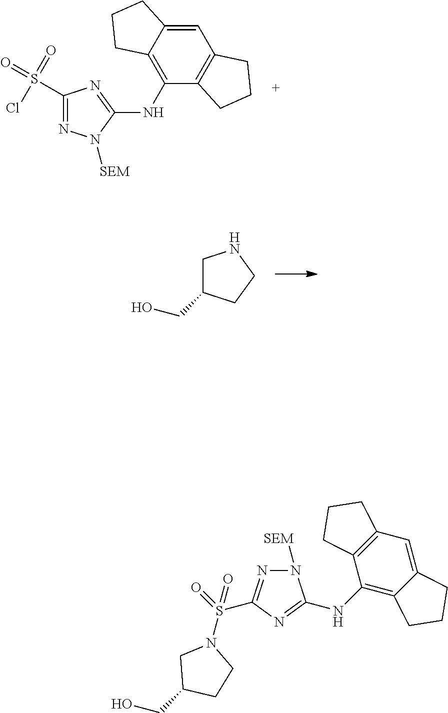 [SMILES: C[Si](C)(C)CCOCn1nc(S(=O)(=O)Cl)nc1Nc1c2c(cc3c1CCC3)CCC2 ; OC[C@H]1CCNC1 ; C[Si](C)(C)CCOCn1nc(S(=O)(=O)N2CC[C@H](CO)C2)nc1Nc1c2c(cc3c1CCC3)CCC2]

Prepared according to the general procedure of (1-((5-((1,2,3,5,6,7-hexahydro-s-indacen-4-yl)amino)-1-((2-(trimethylsilyl) ethoxy)methyl)-1H-1,2,4-triazol-3-yl)sulfonyl)piperidin-3-yl)methanol (Intermediate D3) from (S)-pyrrolidin-3-ylmethanol and 5-((1,2,3,5,6,7-hexahydro-s-indacen-4-yl)amino)-1-((2-(trimethylsilyl)-ethoxy)methyl)-1H-1,2,4-triazole-3-sulfonyl chloride (IntermediateB14) to afford the title compound (0.306 g, 62%) as a yellow oil.

LCMS m/z 534.3 (M+H)⁺ (ES⁺).

¹H NMR (DMSO-d₆) δ 11.05 (s, 1H), 8.8 (s, 1H), 6.98 (s, 1H), 5.50 (s, 2H), 4.68 (t, J=5.2 Hz, 1H), 3.69-3.51 (m, 2H), 3.38 (dd, J=10.0, 7.6 Hz, 1H), 3.36-3.23 (m, 3H), 3.00 (dd, J=9.9, 7.4 Hz, 1H), 2.83 (t, J=7.4 Hz, 4H), 2.65 (t, J=7.4 Hz, 4H), 2.21 (p, J=7.1 Hz, 1H), 2.00-1.93 (m, 4H), 1.85-1.78 (m, 1H), 1.58-1.49 (m, 1H), 0.95-0.83 (m, 2H), −0.03 (s, 9H).

### Intermediate X1: 1-(2,2,2-trifluoroethyl)-1,8-diazaspiro[4.5]decane

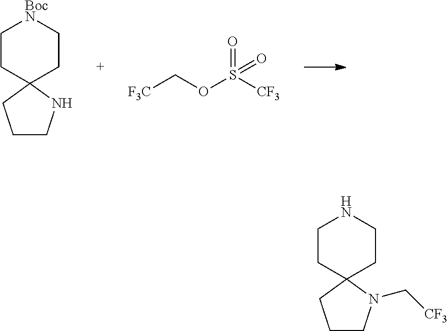 [SMILES: CC(C)(C)OC(=O)N1CCC2(CCCN2)CC1 ; O=S(=O)(OCC(F)(F)F)C(F)(F)F ; FC(F)(F)CN1CCCC12CCNCC2]

To a solution of tert-butyl 1,8-diazaspiro[4.5]decane-8-carboxylate (0.520 g, 2.16 mmol) and DIPEA (0.943 mL, 5.41 mmol) in THF (5 mL) was added 2,2,2-trifluoroethyl trifluoromethanesulfonate (0.468 mL, 3.25 mmol). The reaction was heated at 50° C. for 16 h. Additional 2,2,2-trifluoroethyl trifluoromethanesulfonate (0.468 mL, 3.25 mmol) was added and the reaction was heated at 50° C. for a further 72 h. The reaction mixture diluted with DCM (20 mL) and washed with water (20 mL). The aqueous phase was extracted with DCM (2×10 mL). The combined organic layers were dried (phase separator) and concentrated in vacuo. The residue was dissolved in 1:1 TFA/DCM (5 mL) and stirred at RT for 2 h. The volatiles were evaporated and the resulting residue was loaded onto a column of SCX (12 g) in MeOH. The column was washed with MeOH and the product was eluted with 7 M ammonia in MeOH. The solvent was evaporated to afford the title compound (0.450 g, 92%) as an orange oil.

¹H NMR (CDCl₃) δ 3.13-3.01 (m, 4H), 3.00-2.94 (m, 2H), 2.64 (td, J=12.3, 3.0 Hz, 2H), 1.92-1.71 (m, 4H), 1.47-1.27 (m, 4H). One exchangeable proton not observed.

### Intermediate X2: 1-cyclopropyl-1,8-diazaspiro[4.5]decane

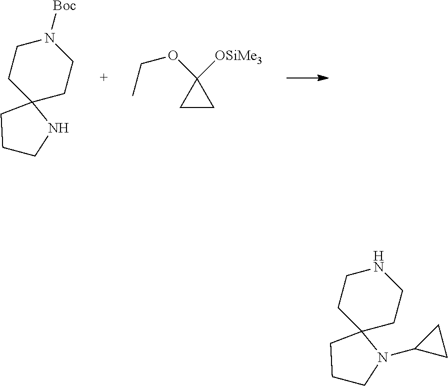

Sodium cyanoborohydride (0.392 g, 6.24 mmol) was added portionwise to a stirred solution of tert-butyl 1,8-diazaspiro[4.5]decane-8-carboxylate (0.500 g, 2.08 mmol), (1-ethoxycyclopropoxy)trimethylsilane (1.26 mL, 6.24 mmol) and acetic acid (0.476 mL, 8.32 mmol) in THF (10 mL) at RT. The mixture was heated to 6° C. for 18 h, then quenched with 2 M NaOH and extracted with DCM (3×30 mL). The combined organic phases were dried (phase separator) and concentrated in vacuo. The crude product was purified by chromatography on silica gel (24 g column, 0-10% MeOH/DCM) to afford tert-butyl 1-cyclopropyl-1,8-diazaspiro[4.5]decane-8-carboxylate. This was dissolved in TFA/DCM (1:1, 10 mL) and stirred at RT for 3 h. The solution was concentrated in vacuo and the resulting crude product was loaded onto a column of SCX (10 g) in MeOH. The column was washed with MeOH and the product was eluted with 7 M ammonia in MeOH. The ammoniacal solution was concentrated in vacuo to afford the title compound (0.147 g, 31%) as a pale yellow oil.

¹H NMR (CD₃OD) δ 3.07-3.00 (m, 2H), 2.93 (dt, J=10.9, 6.9 Hz, 2H), 2.67 (td, J=13.0, 2.6 Hz, 2H), 2.00-1.93 (m, 2H), 1.87-1.80 (m, 2H), 1.81-1.75 (m, 2H), 1.61-1.56 (m, 1H), 1.44-1.38 (m, 2H), 0.57-0.51 (m, 2H), 0.46-0.39 (m, 2H). One exchangeable proton not observed.

### Intermediate X3: 2-cyclopropyl-2,8-diazaspiro[4.5]decane

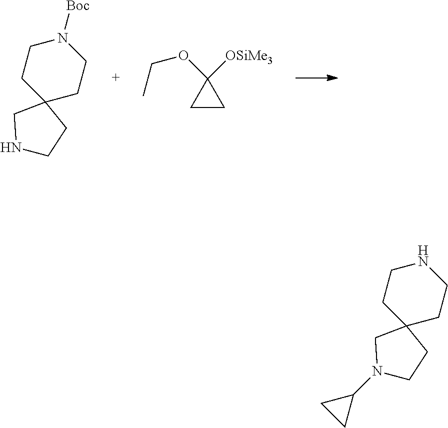

Prepared according to the general procedure of 1-cyclopropyl-1,8-diazaspiro[4.5]decane (Intermediate X2) from tert-butyl 2,8-diazaspiro[4.5]decane-8-carboxylate to afford the title compound (0.188 g, 48%) as a pale yellow oil.

¹H NMR (CD₃OD) δ 2.82-2.68 (m, 6H), 2.59 (s, 2H), 1.74-1.62 (m, 3H), 1.60-1.47 (m, 4H), 0.49-0.36 (m, 4H). One exchangeable proton not observed.

### Intermediate X4: 1-(2-fluoroethyl)-1,8-diazaspiro[4.5]decane

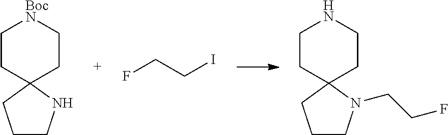

To a stirred solution of tert-butyl 1,8-diazaspiro[4.5]decane-8-carboxylate (0.5 g, 2.08 mmol) in DMF (10 mL) at RT was added K₂CO₃ (0.575 g, 4.16 mmol), followed by 1-fluoro-2-iodoethane (0.27 mL, 2.29 mmol), and the resulting reaction mixture was stirred at 70° C. for 4 h. Additional 1-fluoro-2-iodoethane (0.27 mL, 2.29 mmol) was added and the reaction was heated at 70° C. for a further 18 h. The reaction mixture was cooled to RT and partitioned between EtOAc (20 mL) and water (20 mL). The aqueous phase was separated and re-extracted with EtOAc (2×20 mL). The combined organic phases were washed with brine (50 mL), passed through a phase separator and concentrated in vacuo. The crude product was purified by column chromatography on silica gel (12 g column, 0-15% (0.7 M Ammonia/MeOH)/DCM) to afford tert-butyl 1-(2-fluoroethyl)-1,8-diazaspiro[4.5]decane-8-carboxylate. This was dissolved in TFA/DCM (1:1, 10 mL) and stirred at RT for 16 h. The reaction mixture was concentrated in vacuo and the resulting residue was loaded onto a column of SCX (10 g) in MeOH. The column was washed with MeOH and then the product was eluted with 7 M ammonia in MeOH. The ammoniacal solution was concentrated in vacuo to afford the title compound (0.344 g, 85%) as an orange oil.

¹H NMR (DMSO-d₆) δ 4.43 (dt, J=47.9.54 Hz, 2H), 2.91-2.81 (m, 2H), 2.76 (t, J=7.1 Hz, 2H), 2.71 (dt, J=25.7.54 Hz, 2H), 2.46 (td, J=12.5, 2.2 Hz, 2H), 1.72-1.59 (m, 4H), 1.39 (td, J=12.6, 4.5 Hz, 2H), 1.17-1.10 (m, 2H). One exchangeable proton not observed.

### Intermediate X5: 6-(2,2,2-trifluoroethyl)-2,6-diazaspiro[3.4]octane

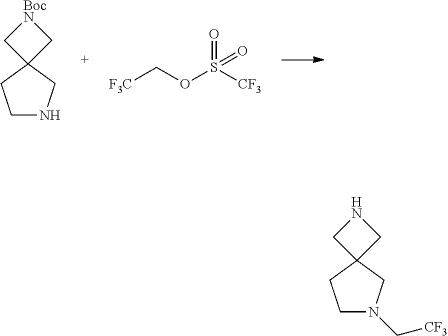 [SMILES: CC(C)(C)OC(=O)N1CC2(CCNC2)C1 ; O=S(=O)(OCC(F)(F)F)C(F)(F)F ; FC(F)(F)CN1CCC2(CNC2)C1]

To a solution of tert-butyl 2,6-diazaspiro[3.4]octane-2-carboxylate (0.5 g, 2.36 mmol) and DIPEA (1.03 mL, 5.89 mmol) in THF (5 mL) was added 2,2,2-trifluoroethyl trifluoromethanesulfonate (1.02 mL, 7.07 mmol). The reaction mixture was heated at 50° C. for 65 h. The reaction mixture was diluted with DCM (20 mL) and washed with water (20 mL). The aqueous phase was re-extracted with DCM (2×10 mL) and the combined organic phases were passed through a phase separator and concentrated in vacuo. The resulting residue was dissolved in 1:1 TFA/DCM (10 mL) and stirred at RT for 2 h. The reaction was concentrated in vacuo and the residue was loaded onto a column of SCX (12 g) in MeOH. The column was washed with MeOH and the product was eluted with 7 M ammonia in MeOH. The ammoniacal solution was concentrated in vacuo to afford the title compound (0.410 g, 85%) as a yellow oil.

¹H NMR (DMSO-d₆) δ 3.35 (s, 4H), 3.27-3.15 (m, 2H), 2.83 (s, 2H), 2.69-2.60 (m, 2H), 1.96-1.87 (m, 2H). One exchangeable proton not observed.

### Intermediate X6: 1-cyclopropyl-1,7-diazaspiro[3.5]nonane

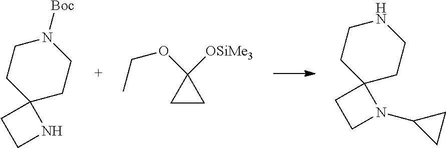

Prepared according to the general procedure of 1-cyclopropyl-1,8-diazaspiro[4.5]decane (Intermediate X2) from tert-butyl 1,7-diazaspiro[3.5]nonane-7-carboxylate to afford the title compound (0.173 g, 44%, 60% purity) as a colourless oil.

¹H NMR (DMSO-d₆) major peaks δ 3.28 (t, J=7.5 Hz, 2H), 3.19-3.11 (m, 2H), 1.91 (t, J=7.3 Hz, 2H), 1.76 (t, J=7.1 Hz, 2H), 0.32-0.24 (m, 2H), 0.18-0.10 (m, 2H).

### Intermediate X7: 1-ethyl-1,8-diazaspiro[4.5]decane

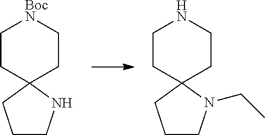 [SMILES: CC(C)(C)OC(=O)N1CCC2(CCCN2)CC1 ; CCN1CCCC12CCNCC2]

To a suspension of tert-butyl 1,8-diazaspiro[4.5]decane-8-carboxylate (0.5 g, 2.08 mmol) and 10% palladium on carbon (0.221 g, 0.208 mmol) in EtOH (17 mL) was added acetaldehyde (3.76 mL, 66.6 mmol) and acetic acid (0.833 mL, 14.6 mmol). The reaction was hydrogenated at 2 bar pressure for 18 h at RT. The reaction mixture was filtered through Celite, washing with MeOH (50 mL). The filtrate was concentrated in vacuo and the residue was purified by chromatography on silica gel (12 g column, 0-15% (0.7 M Ammonia/MeOH)/DCM) to afford tert-butyl 1-ethyl-1,8-diazaspiro[4.5]decane-8-carboxylate. This was dissolved in TFA/DCM (1:1, 10 mL) and stirred at RT for 2 h. The reaction was concentrated in vacuo and the resulting residue was loaded onto a column of SCX (10 g) in MeOH. The column was washed with MeOH and the product was eluted with 7 M ammonia in MeOH. The ammoniacal solution was concentrated in vacuo to afford the title compound (0.167 g, 43%) as an orange oil.

¹H NMR (DMSO-d₆) δ 2.95-2.86 (m, 2H), 2.71-2.62 (m, 2H), 2.42 (q, J=7.0 Hz, 2H), 1.68-1.62 (m, 4H), 1.46 (td, J=12.7, 4.5 Hz, 2H), 1.21-1.08 (m, 2H), 0.98 (t, J=7.1 Hz, 3H). One exchangeable proton not observed, two protons obscured by DMSO-d₆ peak.

### Intermediate X8: 1-ethyl-1,7-diazaspiro[3.5]nonane

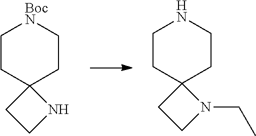 [SMILES: CC(C)(C)OC(=O)N1CCC2(CCN2)CC1 ; CCN1CCC12CCNCC2]

A suspension of tert-butyl 1,7-diazaspiro[3.5]nonane-7-carboxylate (0.5 g, 2.21 mmol) and 10% palladium on carbon (0.235 g, 0.221 mmol) in EtOH/MeCN (2:1.5 mL) was prepared and stirred at RT under 2 atmospheres H₂ for 22 h. The reaction mixture was filtered through Celite, washing with MeOH (50 mL). The filtrate was concentrated in vacuo and the resulting residue was purified by chromatography on silica gel (12 g column, 0-15% (0.7 M Ammonia/MeOH)/DCM) to afford tert-butyl 1-ethyl-1,7-diazaspiro[3.5]nonane-7-carboxylate. This was dissolved in TFA/DCM (1:1, 10 mL) and stirred at RT for 90 min. The reaction was concentrated in vacuo and the resulting residue was loaded onto a column of SCX (10 g) in MeOH. The column was washed with MeOH and the product was eluted with 7 M ammonia in MeOH. The ammoniacal solution was concentrated in vacuo to afford the title compound (0.242 g, 70%) as a yellow oil.

¹H NMR (DMSO-d₆) δ 3.01 (t, J=7.0 Hz, 2H), 2.85-2.75 (m, 2H), 2.42-2.32 (m, 4H), 1.76 (t, J=7.0 Hz, 2H), 1.65-1.57 (m, 2H), 1.41 (td, J=12.4, 4.4 Hz, 2H), 0.84 (t, J=7.2 Hz, 3H). One exchangeable proton not observed.

### Intermediate X9: 2-methyl-2,8-diazaspiro[4.5]decane

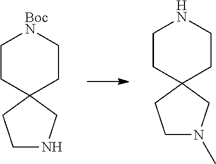 [SMILES: CC(C)(C)OC(=O)N1CCC2(CCNC2)CC1 ; CN1CCC2(CCNCC2)C1]

Prepared according to the general procedure of 1-ethyl-1,8-diazaspiro[4.5]decane (Intermediate X7) from tert-butyl 2,8-diazaspiro[4.5]decane-8-carboxylate and formaldehyde to afford the title compound (0.267 g, 82%) as a yellow oil.

¹H NMR (CD₃OD) δ 2.82-2.69 (m, 4H), 2.59 (t, J=6.9 Hz, 2H), 2.43 (s, 2H), 2.33 (s, 3H), 1.69 (t, J=6.9 Hz, 2H), 1.60-1.48 (m, 4H). One exchangeable proton not observed.

### Intermediate X10: 6-isopropyl-2,6-diazaspiro[3.4]octane

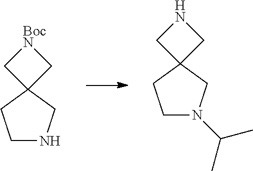

Prepared according to the general procedure of 1-ethyl-1,8-diazaspiro[4.5]decane (Intermediate X7) from tert-butyl 2,6-diazaspiro[3.4]octane-2-carboxylate and acetone to afford the title compound (0.310 g, 81%) as a yellow oil.

¹H NMR (DMSO-d₆) δ 3.34 (s, 4H), 2.63 (s, 2H), 2.44 (t, J=7.1 Hz, 2H), 2.25 (sept, J=6.3 Hz, 1H), 1.86 (t, J=7.0 Hz, 2H), 0.98 (d, J=6.3 Hz, 6H). One exchangeable proton not observed.

### Intermediate X11: 6-(2-fluoroethyl)-2,6-diazaspiro[3.4]octane

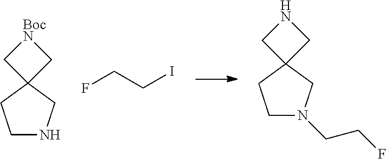

Prepared according to the general procedure of 1-(2-fluoroethyl)-1,8-diazaspiro[4.5]decane (Intermediate X4) from tert-butyl 2,6-diazaspiro[3.4]octane-2-carboxylate to afford the title compound (0.187 g, 68%) as a yellow oil.

¹H NMR (CD₃OD) δ 4.61-4.57 (m, 1H), 4.51-4.47 (m, 1H), 3.65-3.52 (m, 4H), 2.85 (s, 2H), 2.83-2.77 (m, 1H), 2.76-2.72 (m, 1H), 2.64 (t, J=7.1 Hz, 2H), 2.08 (t, J=7.1 Hz, 2H). One exchangeable proton not observed.

### Intermediate X12: 6-cyclopropyl-2,6-diazaspiro[3.4]octane

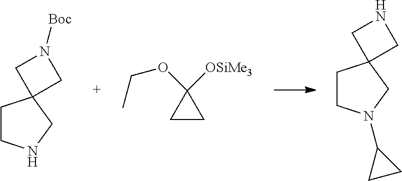

Prepared according to the general procedure of 1-cyclopropyl-1,8-diazaspiro[4.5]decane (Intermediate X2) from tert-butyl 2,6-diazaspiro[3.4]octane-2-carboxylate to afford the title compound (0.343 g, 83%) as a pale yellow oil.

¹H NMR (CD₃OD) δ 3.64-3.57 (m, 4H), 2.94 (s, 2H), 2.76-2.70 (m, 2H), 2.11-2.06 (m, 2H), 1.85-1.65 (m, 1H), 0.53-0.46 (m, 2H), 0.45-0.41 (m, 2H). One exchangeable proton not observed.

### Intermediate X13: 3-(2-methoxypropan-2-yl)pyrrolidine

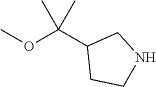

### Step A: tert-butyl 3-(2-hydroxypropan-2-yl)pyrrolidine-1-carboxylate

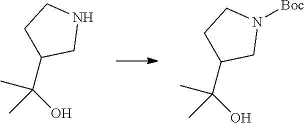

A solution of 2-(pyrrolidin-3-yl)propan-2-ol(209 mg, 1.618 mmol), triethylamine (0.744 mL, 5.34 mmol) and di-tert-butyl dicarbonate (388 mg, 1.78 mmol) in THF (7 mL) was stirred at RT for 3 h, then the volatiles were removed in vacuo to afford the title compound (371 mg, 95%) as a white solid.

¹H NMR (CDCl₃) δ 3.68-3.37 (m, 2H), 3.33-3.20 (m, 1H), 3.20-3.12 (m, 1H), 2.43-2.12 (m, 1H), 1.96-1.69 (m, 2H), 1.47 (s, 9H), 1.25 (s, 6H). One exchangeable proton not observed.

### Step B: tert-butyl 3-(2-methoxypropan-2-yl)pyrrolidine-1-carboxylate

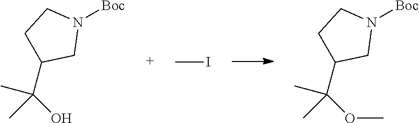

NaH (60% in mineral oil, 780 mg, 19.5 mmol) was added to a solution of tert-butyl 3-(2-hydroxypropan-2-yl)pyrrolidine-1-carboxylate (371 mg, 1.62 mmol) in THF (7 mL) at ° C. and the solution was stirred for 30 min. MeI (0.121 mL, 1.94 mmol) was added and the reaction was stirred at RT for 16 h. The reaction mixture was poured onto ice, diluted with EtOAc/MTBE (1:1, 100 mL), the phases separated and the aqueous further extracted with MTBE (100 mL). The organic phases were combined, dried (MgSO₄) and concentrated in vacuo to afford the title compound (394 mg, 90%) as a yellow oil.

¹H NMR (CDCl₃) δ 3.55-3.45 (m, 1H), 3.45-3.37 (m, 1H), 3.26-3.17 (m, 4H), 3.14 (t, J=10.3 Hz, 1H), 2.45-2.23 (m, 1H), 1.92-1.68 (m, 2H), 1.45 (s, 9H), 1.14 (d, J=1.3 Hz, 6H).

### Step C: 3-(2-methoxypropan-2-yl)pyrrolidine

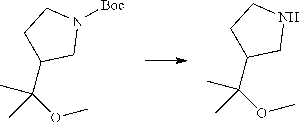

A solution of tert-butyl 3-(2-methoxypropan-2-yl)pyrrolidine-1-carboxylate (394 mg, 1.62 mmol) in DCM (2 mL) and TFA (2 mL) was stirred at RT for 3 h. The volatiles were removed in vacuo and the residue was dissolved in DCM and passed through SCX (3 g) eluting with DCM (20 mL) then 0.7 M NH₃ in MeOH (20 mL). The ammoniacal solution was concentrated in vacuo to afford the title compound (85.7 mg, 33%) as an orange oil.

¹H NMR (CDCl₃) δ 3.18 (s, 3H), 3.10 (s, 1H), 3.03-2.92 (m, 2H), 2.92-2.78 (m, 2H), 2.35-2.16 (m, 1H), 1.93-1.72 (m, 1H), 1.69-1.55 (m, 1H), 1.12 (d, J=3.7 Hz, 6H).

### Intermediate X14: 2-methyl-2,7-diazaspiro[3.5]nonane

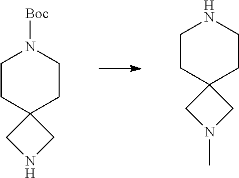

Prepared according to the general procedure of 1-ethyl-1,8-diazaspiro[4.5]decane (Intermediate X7) from tert-butyl 2,7-diazaspiro[3.5]nonane-7-carboxylate and formaldehyde to afford the title compound (0.170 g, 52%) as a colourless oil.

¹H NMR (CDCl₃) δ 2.99 (s, 4H), 2.73 (t, J=5.4 Hz, 4H), 2.32 (s, 3H), 1.72-1.61 (m, 4H). One exchangeable proton not observed.

### Intermediate X15: 6-ethyl-2,6-diazaspiro[3-4]octane

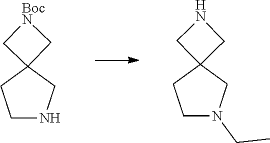

Prepared according to the general procedure of 1-ethyl-1,7-diazaspiro[3.5]nonane (Intermediate X8) from tert-butyl 2,6-diazaspiro[3.4]octane-2-carboxylate to afford the title compound (0.070 g, 50%) as a pale yellow oil.

¹H NMR (CD₃OD) δ 3.70-3.54 (m, 4H), 2.79 (s, 2H), 2.57 (t, J=7.1 Hz, 2H), 2.50 (q, J=7.3 Hz, 2H), 2.09 (t, J=7.1 Hz, 2H), 1.12 (t, J=7.3 Hz, 3H). One exchangeable proton not observed.

### Intermediate X16: 7-methyl-2,7-diazaspiro[3.5]nonane

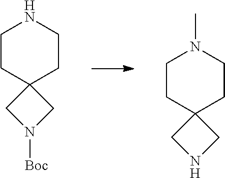

Prepared according to the general procedure of 1-ethyl-1,8-diazaspiro[4.5]decane (Intermediate X7) from tert-butyl 2,7-diazaspiro[3.5]nonane-2-carboxylate and formaldehyde to afford the title compound (57 mg, 37%) as a clear colourless oil. LCMS m/z 141.5 (M+H)⁺ (ES⁺).

### Intermediate X17: 6-methyl-2,6-diazaspiro[3-4]octane

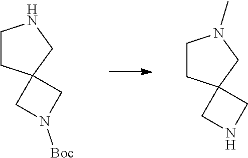

Prepared according to the general procedure of 1-ethyl-1,8-diazaspiro[4.5]decane (Intermediate X7) from tert-butyl 2,6-diazaspiro[3.4]octane-2-carboxylate and formaldehyde to afford the title compound (39 mg, 29%) as a clear colourless oil.

¹H NMR (DMSO-d₆) δ 3.36 (s, 4H), 2.56 (s, 2H), 2.36 (t, J=7.0 Hz, 2H), 2.19 (s, 3H), 1.90 (t, J=7.0 Hz, 2H). One exchangeable proton not observed.

### Intermediate X18: (S)-benzyl methyl(pyrrolidin-3-ylmethyl)carbamate

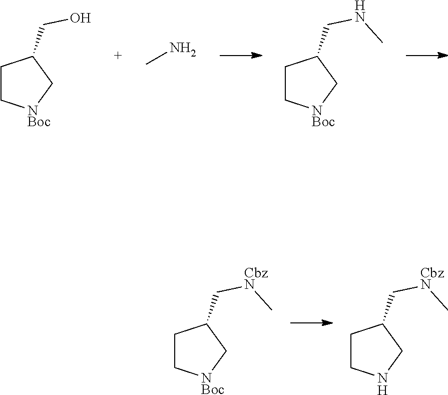

(S)-tert-butyl 3-(hydroxymethyl)pyrrolidine-1-carboxylate (1.00 g, 4.97 mmol) was dissolved in THF (10 mL), to which was added DIPEA (1.215 mL, 6.96 mmol). The reaction mixture was then cooled to 0° C., methanesulfonyl chloride (0.407 mL, 5.22 mmol) added dropwise, and the reaction stirred for 2 h. Potassium iodide (0.082 g, 0.497 mmol) and methylamine (2 M in THF) (37.3 mL, 74.5 mmol) were then added, and the reaction stirred at 6° C. for 18 h. The reaction mixture was concentrated in vacuo and the resulting residue purified by column chromatography on silica gel (24 g column, 0-10% MeOH/DCM) to afford (R)-tert-butyl 3-((methylamino)methyl)-pyrrolidine-1-carboxylate (0.201 g, 0.92 mmol) as a yellow oil. This was dissolved in THF/water (1:1, 5 mL), benzyl (2,5-dioxopyrrolidin-1-yl) carbonate (0.275 g, 1.10 mmol) and sodium hydrogencarbonate (0.112 g, 1.84 mmol) were added and the reaction was stirred at RT for 18 h. The reaction mixture was concentrated in vacuo and the crude product was dissolved in 4 M HCl in dioxane (5 mL) and stirred at RT overnight. The reaction mixture was concentrated in vacuo and the crude product was loaded onto a column of SCX (5 g) in MeOH. The column was washed with MeOH and the product was eluted with 0.7 M ammonia in MeOH. The ammoniacal solution was concentrated in vacuo to afford the title compound (0.132 g, 10.6%) as a brown oil.

¹H NMR (CDCl₃) δ 7.38-7.28 (m, 5H), 5.12 (s, 2H), 3.35-3.21 (m, 2H), 3.07-2.85 (m, 5H), 2.70-2.33 (m, 4H), 1.93-1.79 (m, 1H), 1.50-1.32 (m, 1H)

### Intermediate X19: 1-methyl-1,8-diazaspiro[4.5]decane

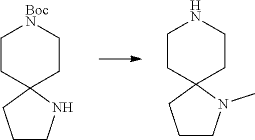 [SMILES: CC(C)(C)OC(=O)N1CCC2(CCCN2)CC1 ; CN1CCCC12CCNCC2]

Prepared according to the general procedure of 1-ethyl-1,8-diazaspiro[4.5]decane (Intermediate X7) from tert-butyl 1,8-diazaspiro[4.5]decane-8-carboxylate and formaldehyde to afford the title compound (455 mg, 68%) as a colourless oil.

¹H NMR (DMSO-d₆) δ 2.92-2.85 (m, 2H), 2.67-2.60 (m, 2H), 2.47 (td, J=12.5, 2.4 Hz, 2H), 2.17 (s, 3H), 1.68-1.58 (m, 4H), 1.44 (td, J=12.6, 4.5 Hz, 2H), 1.12-1.06 (m, 2H). One exchangeable proton not observed.

### Intermediate X20: 1-methyl-1,7-diazaspiro[3.5]nonane

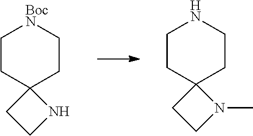

Prepared according to the general procedure of 1-ethyl-1,8-diazaspiro[4.5]decane (Intermediate X7) from tert-butyl 1,7-diazaspiro[3.5]nonane-7-carboxylate and formaldehyde to afford the title compound (376 mg, 61%) as a colourless oil.

¹H NMR (DMSO-d₆) δ 3.04 (t, J=7.0 Hz, 2H), 2.85-2.81 (m, 2H), 2.40 (td, J=12.4, 2.5 Hz, 2H), 2.07 (s, 3H), 1.81 (t, J=7.0 Hz, 2H), 1.62-1.56 (m, 2H), 1.38 (td, J=12.4, 4.4 Hz, 2H). One exchangeable proton not observed.

### Intermediate X21: 1-methyl-4-oxa-1,9-diazaspiro[5.5]undecane

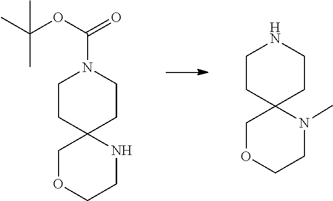 [SMILES: CC(C)(C)OC(=O)N1CCC2(CC1)COCCN2 ; CN1CCOCC12CCNCC2]

Prepared according to the general procedure of 1-ethyl-1,8-diazaspiro[4.5]decane (Intermediate X7) from tert-butyl 4-oxa-1,9-diazaspiro[5.5]undecane-9-carboxylate and formaldehyde to afford the title compound (240 mg, 96%) as a clear oil which solidified upon standing.

¹H NMR (DMSO-d₆) δ 3.58-3.54 (m, 2H), 3.52 (s, 2H), 3.03 (dt, J=13.4, 4.6 Hz, 2H), 2.82 (ddd, J=12.8, 11.7, 3.2 Hz, 2H), 2.56-2.52 (m, 2H), 2.20 (s, 3H), 1.90-1.79 (m, 2H), 1.54-1.46 (m, 2H). One exchangeable proton not observed.

### Intermediate X22: 8-methyl-5-oxa-2,8-diazaspiro[3-5]nonane

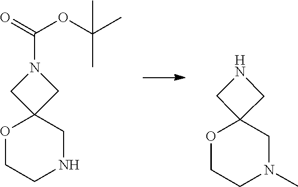

Prepared according to the general procedure of 1-ethyl-1,8-diazaspiro[4.5]decane (Intermediate X7) from tert-butyl 5-oxa-2,8-diazaspiro[3.5]nonane-2-carboxylate and formaldehyde to afford the title compound (330 mg, 82%) as a clear oil which solidified upon standing.

¹H NMR (DMSO-d₆) δ 3.75 (d, J=10.6 Hz, 2H), 3.72 (d, J=10.5 Hz, 2H), 3.62-3.54 (m, 2H), 2.46 (s, 2H), 2.26-2.22 (m, 2H), 2.18 (s, 3H). One exchangeable proton not observed.

### PREPARATION OF EXAMPLES

### Example 1: 5-(benzylthio)-N-(1,2,3,5,6,7-hexahydro-s-indacen-4-yl)-4H-1,2,4-triazol-3-amine

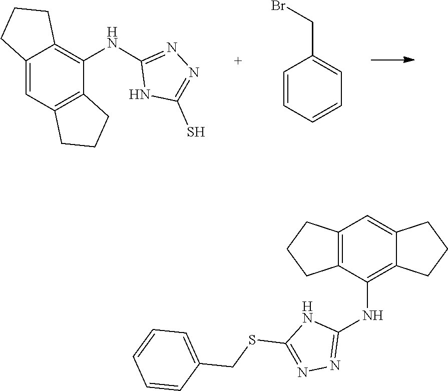 [SMILES: Sc1nnc(Nc2c3c(cc4c2CCC4)CCC3)[nH]1 ; BrCc1ccccc1 ; c1ccc(CSc2nnc(Nc3c4c(cc5c3CCC5)CCC4)[nH]2)cc1]

To a solution of 5-((1,2,3,5,6,7-hexahydro-s-indacen-4-yl)amino)-4H-1,2,4-triazole-3-thiol (Intermediate X7) (100 mg, 367.15 μmol, 1 eq) and NaOH (15 mg, 367.15 μmol, 1 eq) in H₂O (4 mL) was added a solution of (bromomethyl)benzene (63 mg, 367.15 μmol, 1 eq) in DMF (0.5 mL). The mixture was stirred at 20° C. for 12 hours. The mixture was diluted with water (5 mL) and the precipitate was formed. The precipitate was collected to afford the title compound (130 mg, 98% yield, 100% purity on LCMS) as a white solid.

LCMS: m/z 363 (M+H)⁺ (ES⁺).

### Example 2: 5-(benzylsulfonyl)-N-(1,2,3,5,6,7-hexahydro-s-indacen-4-yl)-4H-1,2,4-triazol-3-amine

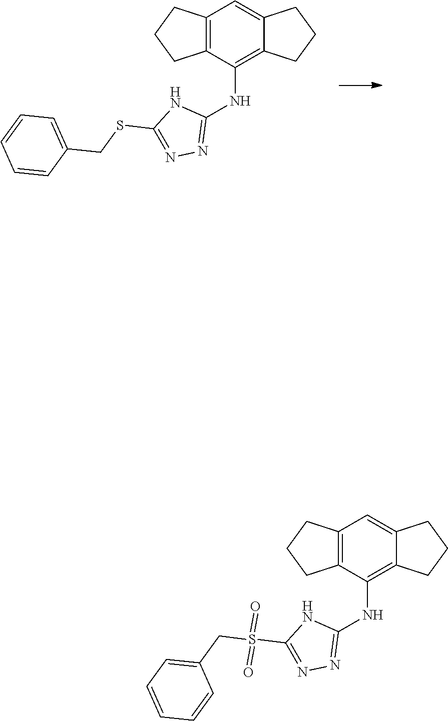 [SMILES: c1ccc(CSc2nnc(Nc3c4c(cc5c3CCC5)CCC4)[nH]2)cc1 ; O=S(=O)(Cc1ccccc1)c1nnc(Nc2c3c(cc4c2CCC4)CCC3)[nH]1]

To a solution of 5-(benzylthio)-N-(1,2,3,5,6,7-hexahydro-s-indacen-4-yl)-4H-1,2,4-triazol-3-amine (Example 1) (130 mg, 358.63 μmol, 1 eq) in DCM (5 mL) was added m-CPBA (124 mg, 717.26 μmol, 85 wt. % in H₂O, 2 eq) at 0° C. The reaction mixture was stirred at 20° C. for 20 minutes. The mixture was washed with water (2×2 mL). The organic layer was dried over Na₂SO₄, filtered and concentrated under reduced pressure. The residue was purified by prep-HPLC (column: Phenomenex Gemini C18 150 mm*25 mm*10 μm; mobile phase: [A: water (0.05% (v/v) HCl); B: MeCN]; B %: 50%-70%, 8.8 min) and then further purified by prep-TLC (SiO₂, 100% ethyl acetate) to afford the title compound (9 mg, 6.36% yield, 100% purity on LCMS) as a grey solid.

¹H NMR (CD₃OD): δ 7.33-7.29 (m, 5H), 7.02 (s, 1H), 4.63 (s, 2H), 2.90 (t, 4H), 2.72 (t, 4H) and 2.10-2.06 (m H). Two exchangable protons not observed.

LCMS: m/z 395 (M+H)⁺ (ES⁺).

### Example 3: 5-((5-(1-(dimethylamino)ethyl)-1-methyl-1H-pyrazol-3-yl)thio)-N-(1,2,3,5,6,7-hexahydro-s-indacen-4-yl)-4H-1,2,4-triazol-3-amine

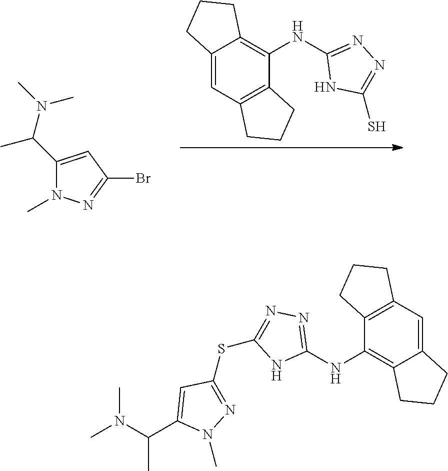 [SMILES: CC(c1cc(Br)nn1C)N(C)C ; Sc1nnc(Nc2c3c(cc4c2CCC4)CCC3)[nH]1 ; CC(c1cc(Sc2nnc(Nc3c4c(cc5c3CCC5)CCC4)[nH]2)nn1C)N(C)C]

To a mixture of 1-(3-bromo-1-methyl-H-pyrazol-5-yl)-N,N-dimethylethanamine (Intermediate L1) (150 mg, 646.22 μmol, 1.2 eq) in dioxane (15 mL) was added 5-((1,2,3,5,6,7-hexahydro-s-indacen-4-yl)amino)-4H-1,2,4-triazole-3-thiol (Intermediate R1) (147 mg, 538.51 mol, 1 eq), N,N′-dimethylethane-1,2-diamine (949 mg, 10.77 mmol, 20 eq) and CuI (205 mg, 1.08 mmol, 2 eq). The reaction mixture was stirred at 100° C. for 16 hours. The mixture was diluted with H₂O (20 mL) and extracted with DCM (3×20 mL). The combined organic layers were washed with brine, dried over Na₂SO₄, filtered and concentrated in vacuum. The residue was purified by silica gel column chromatography (DCM:MeOH, 1:0 to 0:1) to give the title compound (60 mg, 21.04% yield, 80% purity on LCMS) as a yellow solid.

¹H NMR (400 MHz, CDCl₃): δ 6.95 (s, 1H), 6.28 (s, 1H), 4.61-4.53 (m, 1H), 3.91 (s, 3H), 2.87 (t, 4H), 2.76 (t, 4H), 2.19 (s, 6H), 2.09-2.04 (m, 4H) and 1.33-1.29 (m, 3H).

Two exchangable protons not observed.

LCMS: m/z 424.3 (M+H)⁺ (ES⁺).

### Example 4:1-(3-((5-((1,2,3,5,6,7-hexahydro-s-indacen-4-yl)amino)-4H-1,2,4-triazol-3-yl)sulfonyl)-1-methyl-1H-pyrazol-5-yl)-N,N-dimethylethanamineoxide

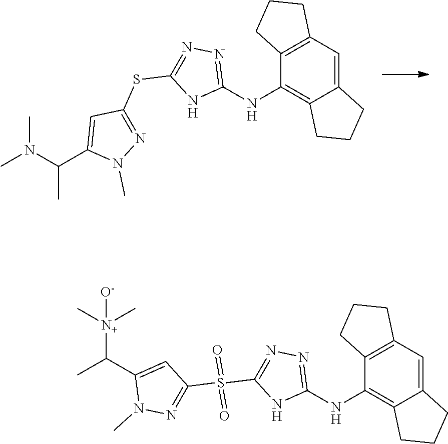 [SMILES: CC(c1cc(Sc2nnc(Nc3c4c(cc5c3CCC5)CCC4)[nH]2)nn1C)N(C)C ; CC(c1cc(S(=O)(=O)c2nnc(Nc3c4c(cc5c3CCC5)CCC4)[nH]2)nn1C)[N+](C)(C)[O-]]

To a solution of 5-((5-(1-(dimethylamino)ethyl)-1-methyl-1H-pyrazol-3-yl)thio)-N-(1,2,3,5,6,7-hexahydro-s-indacen-4-yl)-4H-1,2,4-triazol-3-amine (Example 3) (27 mg, 63.74 μmol, 1 eq) in MeOH (1.4 mL) and H₂O (1.4 mL) was added Na₂CO₃ (20 mg, 191.23 μmol, 3 eq) and Oxone (78 mg, 127.49 μmol, 2 eq). The mixture was stirred at 20° C. for 48 hours. The reaction mixture was filtered and the filtrate was purified by reverse phase flash chromatography (water (0.05% TFA)-MeCN) to give the title compound (5 mg, 12.86% yield, 96% purity on LCMS) as a white solid.

LCMS: m/z 472.1 (M+H)⁺ (ES⁺)

### Example 5: 5-((5-(1-(dimethylamino)ethyl)-1-methyl-1H-pyrazol-3-yl)sulfonyl)-N-(1,2,3,5,6,7-hexahydro-s-indacen-4-yl)-4H-1,2,4-triazol-3-amine

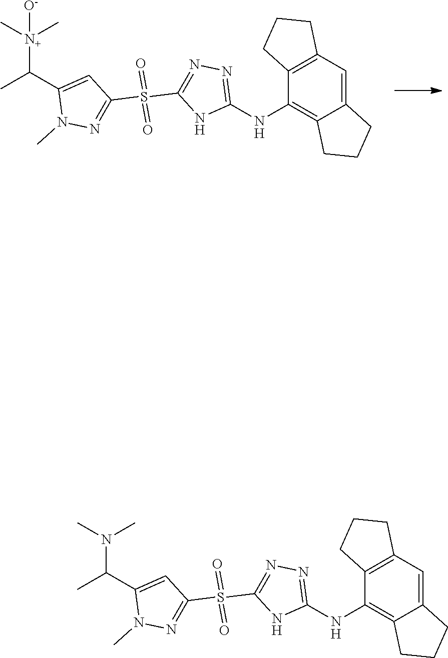 [SMILES: CC(c1cc(S(=O)(=O)c2nnc(Nc3c4c(cc5c3CCC5)CCC4)[nH]2)nn1C)[N+](C)(C)[O-] ; CC(c1cc(S(=O)(=O)c2nnc(Nc3c4c(cc5c3CCC5)CCC4)[nH]2)nn1C)N(C)C]

To a solution of 1-(3-((5-((1,2,3,5,6,7-hexahydro-s-indacen-4-yl)amino)-4H-1,2,4-triazol-3-yl)sulfonyl)-1-methyl-1H-pyrazol-5-yl)-N,N-dimethylethanamine oxide (Example 4) (5 mg, 8.54 μmol, 1 eq) in DCM (0.5 mL) was added 4,4,4′,4′,5,5,5′,5′-octamethyl-2,2′-bi(1,3,2-dioxaborolane) (6 mg, 25.62 μmol, 3 eq). The mixture was stirred at 20° C. for 4 hours. The reaction mixture was concentrated in vacuum. The residue was purified by prep-HPLC (column: Phenomenex Synergi C18, 150 mm*25 mm*10 μm; mobile phase: [A: water (0.1% TFA); B: MeCN]; B %: 15%-45%, min) to give the title compound (0.78 mg, 15.71% yield, 98% purity on LCMS, TFA salt) as a white solid.

¹H NMR (400 MHz, DMSO-d₆): δ 13.24 (s, 1H), 10.21 (br s, 1H), 8.98 (s, 1H), 7.26 (s, 1H), 6.95 (s, 1H), 4.84-4.80 (m, 1H), 3.99 (s, 3H), 2.78 (t, 4H), 2.56 (s, 6H), 2.54 (t, 4H), 1.96-1.92 (m, 4H) and 1.57 (d, 3H).

LCMS: m/z 456.2 (M+H)⁺ (ES⁺)

### Example 6:5-((5-(1-(dimethylamino)ethyl)-1-isopropyl-1H-pyrazol-3-yl)thio)-N-(1,2,3,5,6,7-hexahydro-s-indacen-4-yl)-4H-1,2,4-triazol-3-amine

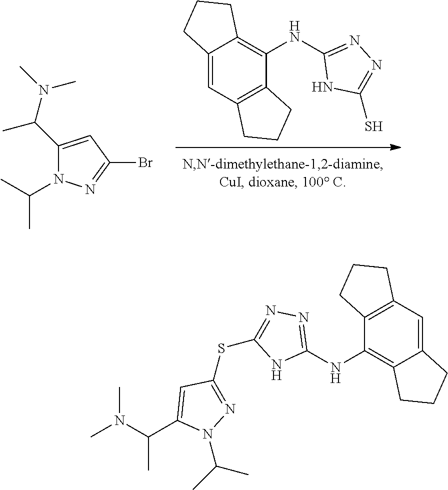 [SMILES: CC(c1cc(Br)nn1C(C)C)N(C)C ; Sc1nnc(Nc2c3c(cc4c2CCC4)CCC3)[nH]1 ; CC(c1cc(Sc2nnc(Nc3c4c(cc5c3CCC5)CCC4)[nH]2)nn1C(C)C)N(C)C]

To a solution of 5-((1,2,3,5,6,7-hexahydro-s-indacen-4-yl)amino)-4H-1,2,4-triazole-3-thiol (Intermediate R1) (10 mg, 550.73 μmol, 1 eq) and 1-(3-bromo-1-isopropyl-H-pyrazol-5-yl)-N,N-dimethylethanamine (Intermediate L2) (172 mg, 660.87 μmol, 1.2 eq) in dioxane (6 mL) was added CuI (210 mg, 1.10 mmol, 2 eq) and N,N′-dimethylethane-1,2-diamine (971 mg, 11.01 mmol, 20 eq). The mixture was stirred at 100° C. for 12 hours. The reaction mixture was quenched with water (40 mL) and extracted with EtOAc (2×80 mL). The organic layer was dried over Na₂SO₄, filtered and concentrated in vacuum. The residue was purified by silica gel column chromatography (SiO₂, Petroleum ether:Ethyl acetate, 1:1 to 0:1) to give the title compound (220 mg, 88.45% yield) as a yellow solid.

¹H NMR (400 MHz, CDCl₃): δ 6.87 (s, 1H), 6.39-6.30 (m, 1H), 6.15 (s, 1H), 4.79-4.69 (s, 1H), 3.74-3.66 (m, 1H), 2.81 (t, 4H), 2.71 (m, 4H), 2.11 (s, 6H), 2.02-1.94 (m, 4H), 1.41 (dd, 6H) and 1.24 (d, 3H). One exchangable proton not observed.

LCMS: m/z 452.3 (M+H)⁺ (ES⁺).

### Example 7: 1-(3-((5-((1,2,3,5,6,7-hexahydro-s-indacen-4-yl)amino)-4H-1,2,4-triazol-3-yl)sulfonyl)-1-isopropyl-1H-pyrazol-5-yl)-N,N-dimethylethanamineoxide

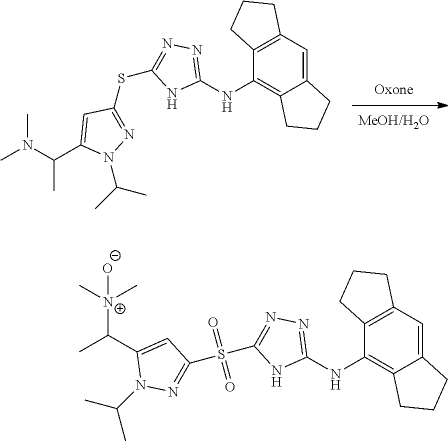 [SMILES: CC(c1cc(Sc2nnc(Nc3c4c(cc5c3CCC5)CCC4)[nH]2)nn1C(C)C)N(C)C ; CC(C)n1nc(S(=O)(=O)c2nnc(Nc3c4c(cc5c3CCC5)CCC4)[nH]2)cc1C(C)[N+](C)(C)[O-]]

To a solution of 5-((5-(1-(dimethylamino)ethyl)-1-isopropyl-1H-pyrazol-3-yl)thio)-N-(1,2,3,5,6,7-hexahydro-s-indacen-4-yl)-4H-1,2,4-triazol-3-amine (Example 6)(80 mg, 177.14 μmol, 1 eq) in MeOH (3 mL) and H₂O (1.5 mL) was added Oxone (490 mg, 797.11 μmol, 4.5 eq) and Na₂CO₃ (56 mg, 531.41 μmol, 3 eq). The reaction mixture was stirred at 10° C. for 12 hours. The reaction mixture was filtered. The filtrate was purified by reverse phase flash chromatography (0.01% TFA in water/MeCN) to give the title compound (35 mg, 39.5% yield) as a yellow solid.

LCMS: m/z 500.0 (M+H)⁺ (ES⁺).

### Example 8: 5-((5-(1-(dimethylamino)ethyl)-1-isopropyl-1H-pyrazol-3-yl)sulfonyl)-N-(1,2,3,5,6,7-hexahydro-s-indacen-4-yl)-4H-1,2,4-triazol-3-amine

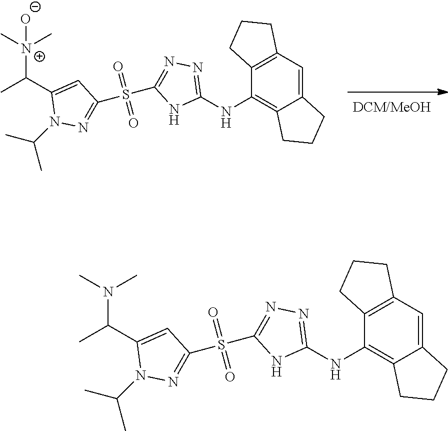 [SMILES: CC(C)n1nc(S(=O)(=O)c2nnc(Nc3c4c(cc5c3CCC5)CCC4)[nH]2)cc1C(C)[N+](C)(C)[O-] ; CC(c1cc(S(=O)(=O)c2nnc(Nc3c4c(cc5c3CCC5)CCC4)[nH]2)nn1C(C)C)N(C)C]

To a solution of 1-(3-((5-((1,2,3,5,6,7-hexahydro-s-indacen-4-yl)amino)-4H-1,2,4-triazol-3-yl)sulfonyl)-1-isopropyl-1H-pyrazol-5-yl)-N,N-dimethylethanamine oxide (Example 7) (30 mg, 60.04 μmol, 1 eq) in DCM (0.5 mL) was added 4,4,4′,4′,5,5,5′,5′-octamethyl-2,2′-bi(1,3,2-dioxaborolane) (46 mg, 180.13 μmol, 3 eq). The reaction mixture was stirred at 15° C. for 0.5 hour. N₂ was bubbled through the reaction mixture to remove the solvent. The residue was purified by prep-HPLC (column: Phenomenex Synergi C18, 150 mm*25 mm*10 μm; mobile phase: [A: water (0.1% TFA); B: MeCN]; B %: 20%-50%, 10 min) and then further purified by prep-HPLC (column: Waters Xbridge, 150 mm*25 mm*5 μm; mobile phase: [A: water (0.05% ammonium hydroxide v/v); B: MeCN]; B %: 10%-40%, 10 min) to give the title compound (8.69 mg, 29.92% yield, 100% purity on LCMS) as a white solid.

¹H NMR (400 MHz, DMSO-d₆): δ 13.16 (br s, 1H), 8.88 (br s, 1H), 6.92 (s, 1H), 6.64 (s, 1H), 4.91-4.87 (s, 1H), 4.00-3.96 (m, 1H), 2.79 (m, 4H), 2.56-2.53 (m, 4H), 2.11 (s, 6H), 1.93-1.90 (m, 4H), 1.38-1.34 (m, 6H) and 1.24 (d, 3H).

LCMS: m/z 484.4 (M+H)⁺ (ES⁺).

### Example A: 5-((1-cyclopropyl-5-(1-(dimethylamino)ethyl)-1H-pyrazol-3-yl)thio)-N-(1,2,3,5,6,7-hexahydro-s-indacen-4-yl)-4H-1,2,4-triazol-3-amine

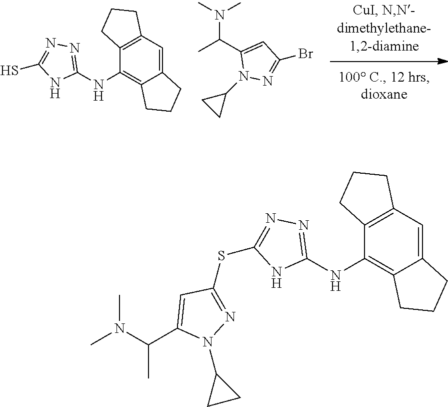

To a solution of 1-(3-bromo-1-cyclopropyl-1H-pyrazol-5-yl)-N,N-dimethylethanamine (Intermediate L3) (150 mg, 569.42 μmol, 1 eq) and 5-(1,2,3,5,6,7-hexahydro-s-indacen-4-ylamino)-4H-1,2,4-triazole-3-thiol (Intermediate R1) (162 mg, 593.68 μmol, 1.04 eq) in dioxane (5 mL) was added CuI (217 mg, 1.14 mmol, 2 eq) and N,N′-dimethylethane-1,2-diamine (1.00 g, 11.39 mmol, 20 eq) at 15° C. The mixture was stirred at 100° C. for 12 hours. The reaction mixture was quenched with NH₃.H₂O (2 mL, 25%), diluted with water (20 mL) and extracted with DCM (3×20 mL). The organic phase was washed with brine (30 mL dried over anhydrous Na₂SO₄, filtered and concentrated. The residue was purified by prep-TLC (SiO₂, DCM:MeOH, 10:1) to give the title compound (80 mg, 31.25% yield) as a brown oil.

¹H NMR (400 MHz, DMSO-d₆): δ 12.33 (br s, 1H), 8.47 (s, 1H), 6.88 (s, 1H), 6.24 (s, 1H), 4.03-3.97 (m, 1H), 3.73-3.70 (m, 1H), 2.80 (t, 4H), 2.61-2.58 (m, 4H), 2.16 (s, 3H), 2.12 (s, 3H), 1.97-1.93 (m, 4H), 1.28-1.25 (m, 4H) and 1.03-1.01 (m, 3H).

LCMS: m/z 450 (M+H)⁺ (ES⁺).

### Example 10: 5-((1-cyclopropyl-5-(1-(dimethylamino)ethyl)-1H-pyrazol-3-yl)sulfinyl)-N-(1,2,3,5,6,7-hexahydro-s-indacen-4-yl)-4H-1,2,4-triazol-3-amine

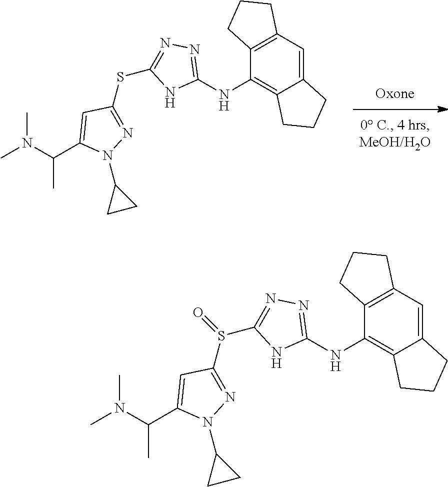 [SMILES: CC(c1cc(Sc2nnc(Nc3c4c(cc5c3CCC5)CCC4)[nH]2)nn1C1CC1)N(C)C ; CC(c1cc(S(=O)c2nnc(Nc3c4c(cc5c3CCC5)CCC4)[nH]2)nn1C1CC1)N(C)C]

To a solution of 5-((1-cyclopropyl-5-(1-(dimethylamino)ethyl)-1H-pyrazol-3-yl)thio)-N-(1,2,3,5,6,7-hexahydro-s-indacen-4-yl)-4H-1,2,4-triazol-3-amine (Example 9) (20 mg, 44.48 μmol, 1 eq) in MeOH (0.5 mL) and H₂O (0.5 mL) was added Oxone (55 mg, 88.97 μmol, 2 eq). The mixture was stirred at 0° C. for 4 hours. The reaction mixture was filtered. The filtrate was purified by reverse phase flash chromatography (0.01% TFA in water/MeCN) and then further purified by prep-HPLC (column: Waters Xbridge, 150 mm*25 mm*5 μm; mobile phase: [A: water (0.05% ammonium hydroxide v/v); B: MeCN]; B %: 15%-45%, 10 min) to give the title compound (9.05 mg, 4370% yield, 100% purity on LCMS) as a white solid.

¹H NMR (400 MHz, DMSO-d₆): δ 12.95 (br s, 1H), 8.83 (s, 1H), 6.94 (s, 1H), 6.52 (d, 1H), 4.07-4.04 (m, 1H), 3.86-3.84 (m, 1H), 2.81 (t, 4H), 2.59 (t, 4H), 2.16 (s, 3H), 2.12 (s, 3H), 1.97-1.93 (m, 4H), 1.28-1.25 (m, 4H) and 1.03-1.01 (m, 3H).

LCMS: m/z 466.3 (M+H)⁺ (ES⁺).

### Example 11: N-(1,2,3,5,6,7-hexahydro-s-indacen-4-yl)-5-((5-(3-methoxyoxetan-3-yl)-1-methyl-1H-pyrazol-3-yl)thio)-4H-1,2,4-triazol-3-amine

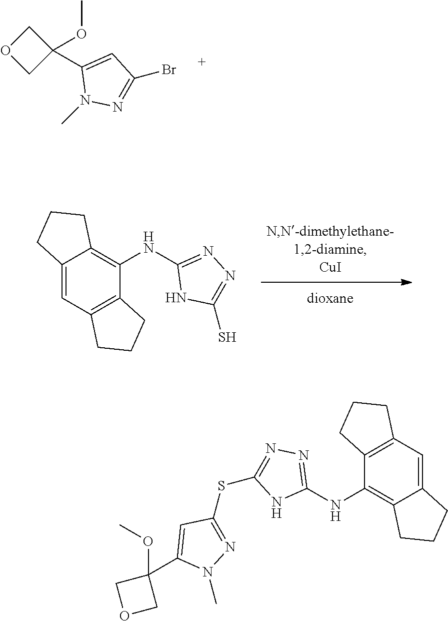 [SMILES: COC1(c2cc(Br)nn2C)COC1 ; Sc1nnc(Nc2c3c(cc4c2CCC4)CCC3)[nH]1 ; COC1(c2cc(Sc3nnc(Nc4c5c(cc6c4CCC6)CCC5)[nH]3)nn2C)COC1]

To a solution of 3-bromo-5-(3-methoxyoxetan-3-yl)-1-methyl-1H-pyrazole (Intermediate L4) (100 mg, 404.71 μmol, 1 eq) and 5-((1,2,3,5,6,7-hexahydro-s-indacen-4-yl)amino)-4H-1,2,4-triazole-3-thiol (Intermediate R1) (110 mg, 404.71 μmol, 1 eq) in dioxane (1 mL) was added CuI (154 mg, 809.43 μmol, 2 eq) and N,N′-dimethylethane-1,2-diamine (713 mg, 8.09 mmol, 20 eq). The reaction mixture was stirred at 100° C. for 12 hours. The reaction mixture was then concentrated in vacuum. The residue was purified by silica gel column chromatography (Petroleum ether:Ethyl acetate, 1:1 to 0:1) to give the title compound (150 mg, 80.29% yield, 95% purity on LCMS) as a yellow solid.

¹H NMR (400 MHz, CD₃OD): δ 6.86 (s, 1H), 6.55 (s, 1H), 4.80-4.75 (m, 4H), 3.62 (s, 3H), 2.97 (s, 3H), 2.76 (t, 4H), 2.61 (t, 4H) and 1.98-1.91 (m, 4H). Two exchangeable protons not observed.

LCMS: m/z 439.2 (M+H)⁺ (ES⁺).

### Example 12: N-(1,2,3,5,6,7-hexahydro-s-indacen-4-yl)-5-((5-(3-methoxyoxetan-3-yl)-1-methyl-1H-pyrazol-3-yl)sulfonyl)-4H-1,2,4-triazol-3-amine

### Example 26: N-(1,2,3,5,6,7-hexahydro-s-indacen-4-yl)-5-((5-(3-methoxyoxetan-3-yl)-1-methyl-1H-pyrazol-3-yl)sulfinyl)-4H-1,2,4-triazol-3-amine

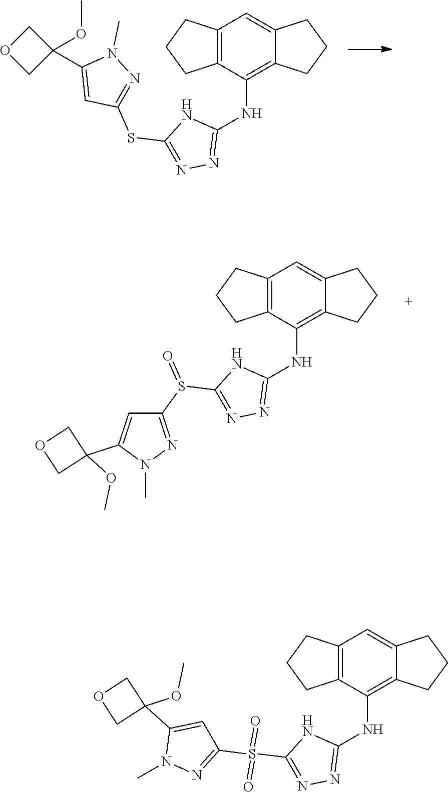 [SMILES: COC1(c2cc(Sc3nnc(Nc4c5c(cc6c4CCC6)CCC5)[nH]3)nn2C)COC1 ; COC1(c2cc(S(=O)c3nnc(Nc4c5c(cc6c4CCC6)CCC5)[nH]3)nn2C)COC1 ; COC1(c2cc(S(=O)(=O)c3nnc(Nc4c5c(cc6c4CCC6)CCC5)[nH]3)nn2C)COC1]

To a solution of N-(1,2,3,5,6,7-hexahydro-s-indacen-4-yl)-5-((5-(3-methoxyoxetan-3-yl)-1-methyl-1H-pyrazol-3-yl)thio)-4H-1,2,4-triazol-3-amine (Example 11) (25 mg, 57.01 μmol, 1 eq) in AcOH (0.5 mL) was added H₂O₂ (2.95 g, 26.02 mmol, 30 wt. % in water, 456.41 eq). The reaction mixture was stirred at 25° C. for 48 hours. The reaction mixture was purified by reverse phase flash chromatography (0.1% TFA in water/MeCN) and then further purified by prep-HPLC (column: Phenomenex Synergi C18, 150 mm*25 mm*10 μm; mobile phase: [A: water (0.1% TFA); B: MeCN]; B %: 40%-64%, 10 minutes) to give the title compound of Example 12 (1.02 mg, 3.73% yield, 98% purity on LCMS) as a white solid and the title compound of Example 26 (13.87 mg, 53.53% yield, 100% purity on LCMS) as a white solid.

### Example 12

¹H NMR (400 MHz, DMSO-d₆): δ 13.19 (s, 1H), 8.99 (s, 1H), 7.24 (s, 1H), 6.95 (s, 1H), 4.90 (d, 2H), 4.79 (d, 2H), 3.77 (s, 3H), 2.98 (s, 3H), 2.81 (t, 4H), 2.57 (t, 4H) and 1.98-1.92 (m, 4H).

LCMS: m/z 471.2 (M+H)⁺ (ES⁺).

### Example 26

¹H NMR (400 MHz, DMSO-d₆): δ 12.98 (br s, 1H), 8.87 (s, 1H), 6.98 (s, 1H), 6.94 (s, 1H), 4.88-4.76 (m, 4H), 3.73 (s, 3H), 2.98 (s, 3H), 2.80 (t, 4H), 2.60 (t, 4H) and 1.97-1.92 (m, 4H).

LCMS: m/z 455.2 (M+H)⁺ (ES⁺).

### Example 13: N-(1,2,3,5,6,7-hexahydro-s-indacen-4-yl)-5-((1-isopropyl-5-(3-methoxyoxetan-3-yl)-1H-pyrazol-3-yl)thio)-4H-1,2,4-triazol-3-amine

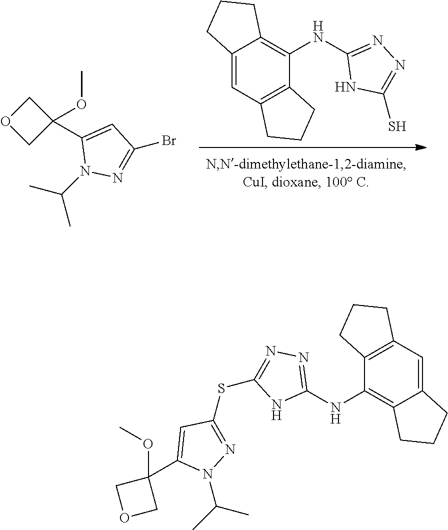 [SMILES: COC1(c2cc(Br)nn2C(C)C)COC1 ; Sc1nnc(Nc2c3c(cc4c2CCC4)CCC3)[nH]1 ; COC1(c2cc(Sc3nnc(Nc4c5c(cc6c4CCC6)CCC5)[nH]3)nn2C(C)C)COC1]

To a solution of 3-bromo-1-isopropyl-5-(3-methoxyoxetan-3-yl)-1H-pyrazole (Intermediate L5) (96 mg, 348.79 μmol, 1 eq) in dioxane (2 mL) were added 5-((1,2,3,5,6,7-hexahydro-s-indacen-4-yl)amino)-4H-1,2,4-triazole-3-thiol (Intermediate R1) (100 mg, 348.79 μmol, 1 eq), CuI (133 mg, 697.59 μmol, 2 eq) and N,N′-dimethylethane-1,2-diamine (615 mg, 6.98 mmol, 20 eq). The reaction mixture was stirred at 100° C. for 12 hours. The mixture was diluted with H₂O (10 mL) and extracted with EtOAc (3×20 mL). The combined organic layers were washed with brine (2×10 mL), dried over Na₂SO₄, filtered and concentrated under reduced pressure. The residue was purified by silica gel column chromatography (SiO₂, Petroleum ether:Ethyl acetate, 5:1 to 0:1) to give the title compound (110 mg, 60.83% yield, 90% purity on LCMS) as a yellow solid.

¹H NMR (400 MHz, DMSO-d₆): δ 12.49 (s, 1H), 8.56 (s, 1H), 6.89 (s, 1H), 6.57 (s, 1H), 4.80-4.75 (m, 4H), 4.15-4.11 (m, 1H), 2.97 (s, 3H), 2.79-2.77 (m, 4H), 2.61-2.60 (m, 4H), 1.96-1.92 (m, 4H) and 1.32 (d, 6H).

LCMS: m/z 467.3 (M+H)⁺ (ES⁺).

### Example 14: N-(1,2,3,5,6,7-hexahydro-s-indacen-4-yl)-5-((1-isopropyl-5-(3-methoxyoxetan-3-yl)-1H-pyrazol-3-yl)sulfonyl)-4H-1,2,4-triazol-3-amine

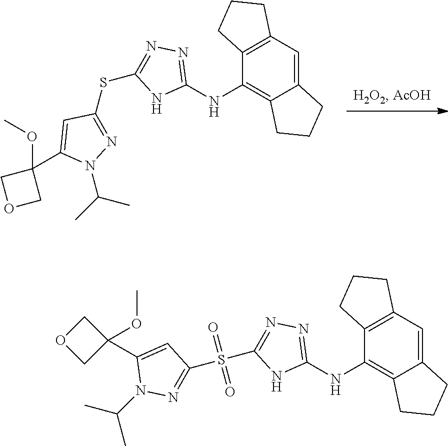 [SMILES: COC1(c2cc(Sc3nnc(Nc4c5c(cc6c4CCC6)CCC5)[nH]3)nn2C(C)C)COC1 ; COC1(c2cc(S(=O)(=O)c3nnc(Nc4c5c(cc6c4CCC6)CCC5)[nH]3)nn2C(C)C)COC1]

To a solution of N-(1,2,3,5,6,7-hexahydro-s-indacen-4-yl)-5-((1-isopropyl-5-(3-methoxyoxetan-3-yl)-1H-pyrazol-3-yl)thio)-4H-1,2,4-triazol-3-amine (Example 13) (25 mg, 53.58 μmol, 1 eq) in AcOH (1.5 mL) and was added H₂O₂ (1.05 g, 9.25 mmol, 30 wt. % in water, 172.57 eq). The reaction mixture was stirred at 25° C. for 2 hours. The reaction mixture was purified by reverse phase flash chromatography (0.1% of TFA in water/MeCN) and then further purified by prep-HPLC (column: Waters Xbridge, 150 mm*25 mm*5 μm; mobile phase: [A: water (0.05% ammonium hydroxide v/v); B: MeCN]; B %: 1%-42%,14 min) to give the title compound (1.72 mg, 6.31% yield, 98% purity on LCMS) as a white solid.

¹H NMR (400 MHz, DMSO-d₆): δ 13.19 (s, 1H), 8.93 (s, 1H), 7.16 (s, 1H), 6.94 (s, 1H), 4.89-4.80 (m, 4H), 4.30-4.25 (m, 1H), 2.98 (s, 3H), 2.80 (t, 4H), 2.52 (t, 4H), 1.95-1.91 (m, 4H) and 1.34 (d, 6H).

LCMS: m/z 499.3 (M+H)⁺ (ES⁺).

### Example 15:5-((1-cyclopropyl-5-(3-methoxyoxetan-3-yl)-1H-pyrazol-3-yl)thio)-N-(1,2,3,5,6,7-hexahydro-s-indacen-4-yl)-4H-1,2,4-triazol-3-amine

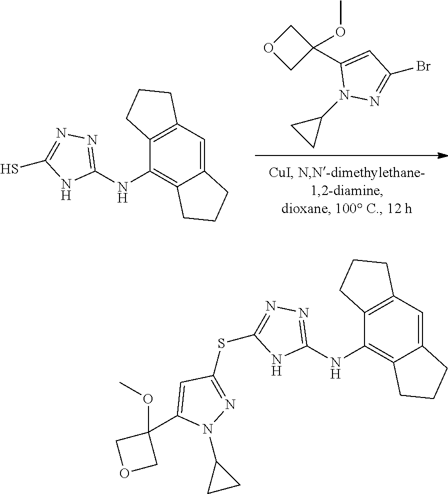 [SMILES: Sc1nnc(Nc2c3c(cc4c2CCC4)CCC3)[nH]1 ; COC1(c2cc(Br)nn2C2CC2)COC1 ; COC1(c2cc(Sc3nnc(Nc4c5c(cc6c4CCC6)CCC5)[nH]3)nn2C2CC2)COC1]

To a solution of 5-((1,2,3,5,6,7-hexahydro-s-indacen-4-yl)amino)-4H-1,2,4-triazole-3-thiol (Intermediate R1) (150 mg, 550.73 μmol, 1 eq) and 3-bromo-1-cyclopropyl-5-(3-methoxyoxetan-3-yl)-1H-pyrazole (Intermediate L6) (180 mg, 660.88 μmol, 1.2 eq) in dioxane (6 mL) was added CuI (210 mg, 1.10 mmol, 2 eq) and N,N′-dimethylethane-1,2-diamine (971 mg, 11.01 mmol, 20 eq). The reaction mixture was stirred at 100° C. for 12 hours. The reaction mixture was poured into water (40 mL) and extracted with EtOAc (2×80 mL). The organic layer was dried over Na₂SO₄, filtered and concentrated in vacuum. The residue was purified by silica gel column chromatography (SiO₂, Petroleum ether:Ethyl acetate, 2:1 to 1:1) to give the title compound (130 mg, 50.81% yield) as a yellow solid.

¹H NMR (400 MHz, CDCl₃): δ 6.90 (s, 1H), 6.42 (s, 1H), 6.33 (br, s, 1H), 4.86-4.80 (m, 4H), 3.43-3.38 (m, 1H), 3.02 (s, 3H), 2.81 (t, 4H), 2.69 (t, 4H), 2.01-1.95 (m, 4H), 1.21-1.15 (m, 2H) and 0.91-0.87 (m, 2H). One exchangable proton not observed.

LCMS: m/z 465.3 (M+H)⁺ (ES⁺).

### Example 16: 5-((1-cyclopropyl-5-(3-methoxyoxetan-3-yl)-1H-pyrazol-3-yl)sulfonyl)-N-(1,2,3,5,6,7-hexahydro-s-indacen-4-yl)-4H-1,2,4-triazol-3-amine

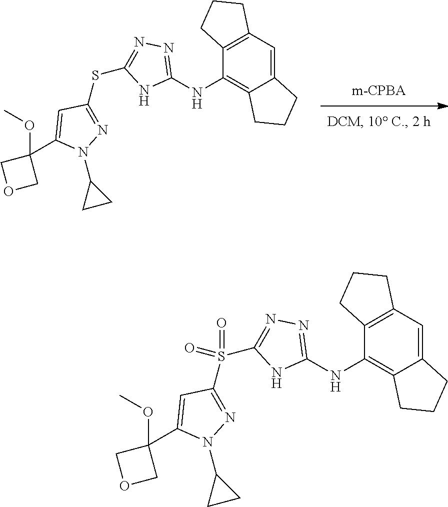 [SMILES: COC1(c2cc(Sc3nnc(Nc4c5c(cc6c4CCC6)CCC5)[nH]3)nn2C2CC2)COC1 ; COC1(c2cc(S(=O)(=O)c3nnc(Nc4c5c(cc6c4CCC6)CCC5)[nH]3)nn2C2CC2)COC1]

To a solution of 5-((1-cyclopropyl-5-(3-methoxyoxetan-3-yl)-1H-pyrazol-3-yl)thio)-N-(1,2,3,5,6,7-hexahydro-s-indacen-4-yl)-4H-1,2,4-triazol-3-amine (Example 15) (100 mg, 215.25 μmol, 1 eq) in DCM (5 mL) was added m-CPBA (175 mg, 860.99 μmol, 85 wt. % in H₂O, 4 eq). The reaction mixture was stirred at 10° C. for 2 hours. N₂ was bubbled through the reaction mixture to remove the solvent. The residue was purified by reverse phase flash chromatography (0.01% NH₃.H₂O/MeCN) and then purified by prep-HPLC (column: Phenomenex Synergi C18, 150 mm*25 mm*10 μm; mobile phase: [A: water (0.1% TFA); B: MeCN]; B %: 45%-69%,10 min) to give the title compound (4.11 mg, 3.85% yield, 100% purity on LCMS) as a white solid.

¹H NMR (400 MHz, DMSO-d₆): δ 13.17 (s, 1H), 8.97 (s, 1H), 7.24 (s, 1H), 6.95 (s, 1H), 4.96 (d, 2H), 4.81 (d, 2H), 3.63-3.60 (m, 1H), 3.01 (s, 3H), 2.81 (t, 4H), 2.55 (t, 4H), 1.97-1.92 (m, 4H), 1.11-1.09 (m, 2H) and 1.01-0.98 (m, 2H).

LCMS: m/z 497.1 (M+H)⁺ (ES⁺).

### Example 17: N-(1,2,3,5,6,7-hexahydro-s-indacen-4-yl)-5-((1-(2-methoxy-2-methylpropyl)-1H-pyrazol-3-yl)thio)-4H-1,2,4-triazol-3-amine

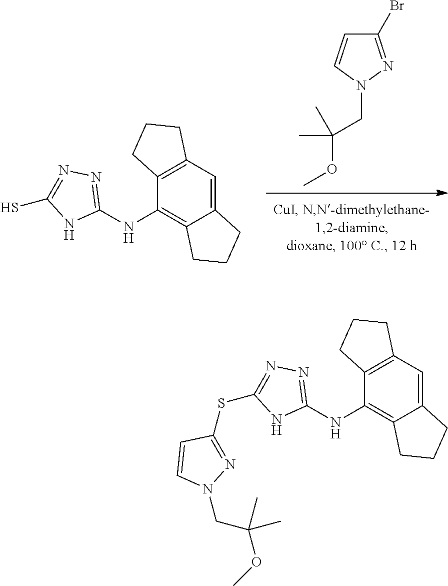 [SMILES: Sc1nnc(Nc2c3c(cc4c2CCC4)CCC3)[nH]1 ; COC(C)(C)Cn1ccc(Br)n1 ; COC(C)(C)Cn1ccc(Sc2nnc(Nc3c4c(cc5c3CCC5)CCC4)[nH]2)n1]

To a solution of 5-((1,2,3,5,6,7-hexahydro-s-indacen-4-yl)amino)-4H-1,2,4-triazole-3-thiol (Intermediate R1) (200 mg, 734.30 μmol, 1 eq) and 3-bromo-1-(2-methoxy-2-methylpropyl)-1H-pyrazole (Intermediate L7) (171 mg, 734.30 μmol, 1 eq) in dioxane (10 mL) was added CuI (280 mg, 1.47 mmol, 2 eq) and N¹,N²-dimethylethane-1,2-diamine (1.29 g, 14.69 mmol, 20 eq). The mixture was stirred at 100° C. for 12 hours. The reaction mixture was poured into water (30 mL) and extracted with EtOAc (3×80 mL). The combined organic layers were dried over Na₂SO₄, filtered and concentrated in vacuum. The residue was purified by silica gel column chromatography (SiO₂, Petroleum ether:Ethyl acetate, 2:1 to 0:1) to give the title compound (150 mg, 48.11% yield) as a yellow oil.

¹H NMR (400 MHz, DMSO-d₆): δ 12.37 (s, 1H), 8.61 (s, 1H), 7.76 (s, 1H), 6.96 (s, 1H), 6.42 (s, 1H), 4.15 (s, 2H), 3.21 (s, 3H), 2.85 (t, 4H), 2.66 (t, 4H), 2.03-1.97 (m, 4H) and 1.11 (s, 6H).

LCMS: m/z 425.3 (M+H)⁺ (ES⁺).

### Example 18:N-(1,2,3,5,6,7-hexahydro-s-indacen-4-yl)-5-((1-(2-methoxy-2-methylpropyl)-1H-pyrazol-3-yl)sulfonyl)-4H-1,2,4-triazol-3-amine

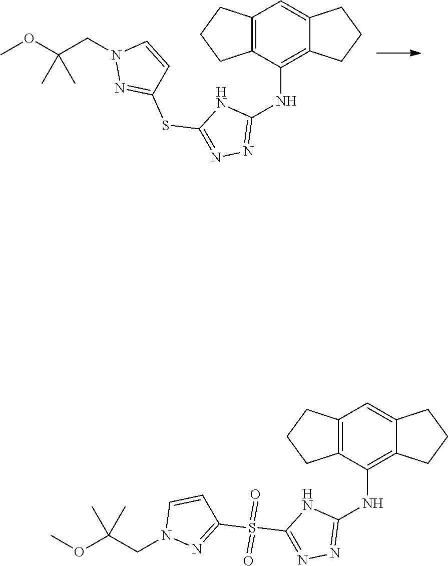 [SMILES: COC(C)(C)Cn1ccc(Sc2nnc(Nc3c4c(cc5c3CCC5)CCC4)[nH]2)n1 ; COC(C)(C)Cn1ccc(S(=O)(=O)c2nnc(Nc3c4c(cc5c3CCC5)CCC4)[nH]2)n1]

To a solution of N-(1,2,3,5,6,7-hexahydro-s-indacen-4-yl)-5-((1-(2-methoxy-2-methylpropyl)-1H-pyrazol-3-yl)thio)-4H-1,2,4-triazol-3-amine (Example 17) (60 mg, 141.32 μmol, 1 eq) in MeOH (2 mL) and H₂O (2 mL) was added Oxone (521 mg, 847.93 μmol, 6 eq). The reaction mixture was stirred at 25° C. for 16 hours. The reaction mixture was filtered and the filtrate was purified by prep-HPLC (column: Phenomenex Synergi C18, 150 mm*25 mm*10 μm; mobile phase: [A: water (0.05% HCl B: MeCN]; B %: 42%-58%, 9 min) and then purified by prep-HPLC (column: Phenomenex Synergi C18, 150 mm*25 mm*10 μm; mobile phase: [A: water (0.1% TFA); B: MeCN]; B %: 40%-70%, 10 min) to give the title compound (0.78 mg, 1.12% yield) as a yellow solid.

¹H NMR (400 MHz, DMSO-d₆): δ 13.04 (s, 1H), 8.94 (s, 1H), 8.27 (s, 1H), 7.88 (s, 1H), 6.95 (s, 1H), 4.22 (s, 2H), 3.15 (s, 3H), 2.81 (t, 4H), 2.58-252 (m, 4H), 1.96-1.92 (m, 4H) and 1.06 (s, 6H).

LCMS: m/z 457.2 (M+H)⁺ (ES⁺).

### Example 19: tert-butyl 3-((5-((1,2,3,5,6,7-hexahydro-s-indacen-4-yl)amino)-4H-1,2,4-triazol-3-yl)thio)azetidine-1-carboxylate

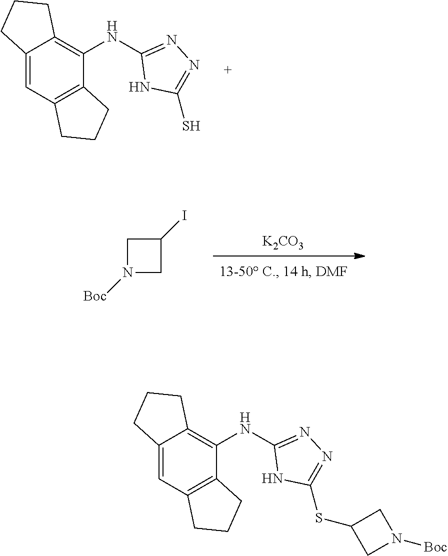

To a solution of 5-((1,2,3,5,6,7-hexahydro-s-indacen-4-yl)amino)-4H-1,2,4-triazole-3-thiol (Intermediate R1) (250 mg, 917.88 μmol, 1 eq) in DMF (5 mL) was added K₂CO₃ (152 mg, 1.10 mmol, 1.2 eq) and tert-butyl 3-iodoazetidine-1-carboxylate (260 mg, 918 μmol, 1 eq). The mixture was stirred at 13° C. for 12 hours. The reaction mixture was heated to 50° C. for 2 hours. The reaction mixture was poured into water (50 mL) and the mixture was stirred for another 1 hour. The mixture was filtered and the filter cake was dried in vacuum to give the crude product. The crude product was triturated with a mixture of PE and EtOAc (ratio 10:1, 20 mL) to give the title compound (300 mg, 76.44% yield) as a red solid.

¹H NMR (400 MHz, DMSO-d₆): δ 12.45 (br s, 1H), 8.53 (br s, 1H), 6.90 (s, 1H), 4.25-4.14 (m, 3H), 3.77-3.74 (m, 2H), 2.81 (t, 4H), 2.62 (t, 4H), 1.99-1.93 (m, 4H) and 1.38 (s, 9H).

LCMS: m/z 428.2 (M+H)⁺ (ES⁺).

### Example 20: tert-butyl 3-((5-((1,2,3,5,6,7-hexahydro-s-indacen-4-yl)amino)-4H-1,2,4-triazol-3-yl)sulfonyl)azetidine-1-carboxylate

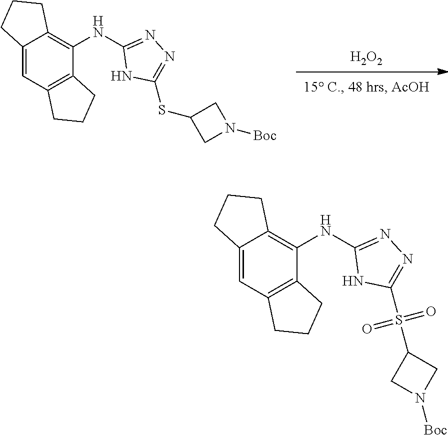 [SMILES: CC(C)(C)OC(=O)N1CC(Sc2nnc(Nc3c4c(cc5c3CCC5)CCC4)[nH]2)C1 ; "H2O2" ; CC(C)(C)OC(=O)N1CC(S(=O)(=O)c2nnc(Nc3c4c(cc5c3CCC5)CCC4)[nH]2)C1]

To a solution of tert-butyl 3-((5-((1,2,3,5,6,7-hexahydro-s-indacen-4-yl)amino)-4H-1,2,4-triazol-3-yl)thio)azetidine-1-carboxylate (Example 19) (300 mg, 701.65 μmol, 1 eq) in AcOH (5 mL) was added H₂O₂ (6.36 g, 56.13 mmol, 30 wt. % in water, 80 eq). The mixture was stirred at 15° C. for 48 hours. The reaction mixture was poured into water (40 mL) and extracted with EtOAc (2×80 mL). The combined organic layers were washed with brine (2×20 mL), dried over Na₂SO₄, filtered and concentrated in vacuum. The residue was purified by reverse phase flash chromatography (0.01% NH₃.H₂O/CH₃CN) to give the title compound (140 mg, 43.42% yield) as an off-white solid.

¹H NMR (400 MHz, DMSO-d₆): δ 8.99 (s, 1H), 6.97 (s, 1H), 4.47-4.40 (m, 1H), 4.15-4.07 (m, 4H), 2.83 (t, 4H), 2.63 (t, 4H), 2.00-1.94 (m, 4H) and 1.38 (s, 9H). One exchangable proton not observed.

LCMS: m/z 919.6 (2M+H)⁺ (ES⁺).

### Example 21: 5-(azetidin-3-ylsulfonyl)-N-(1,2,3,5,6,7-hexahydro-s-indacen-4-yl)-4H-1,2,4-triazol-3-amine

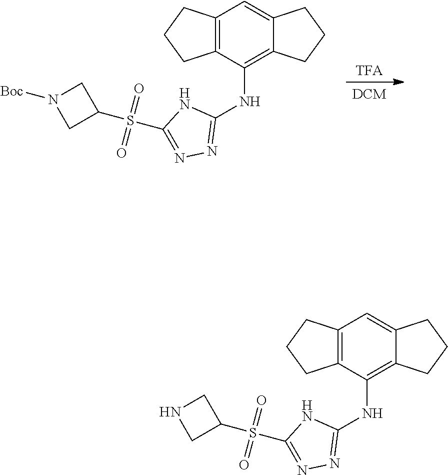

To a solution of tert-butyl 3-((5-((1,2,3,5,6,7-hexahydro-s-indacen-4-yl)amino)-4H-1,2,4-triazol-3-yl)sulfonyl)azetidine-1-carboxylate (Example 20) (100 mg, 217.60 μmol, 1 eq) in DCM (2.5 mL) was added TFA (1.24 g, 10.88 mmol, 50 eq). The reaction mixture was stirred at 10° C. for 2 hours. The reaction mixture was concentrated in vacuum to give the title compound (100 mg, 97.06% yield, TFA salt) as a yellow oil.

LCMS: m/z 360.2 (M+H)⁺ (ES⁺).

¹H NMR (DMSO-d₆) δ 9.00 (s, 1H), 6.96 (s, 1H), 4.53 (tt, J=8.5, 6.7 Hz, 1H), 3.85 (dd, J=9.1, 6.7 Hz, 2H), 3.67 (app t, J=8.8 Hz, 2H), 2.83 (t, J=7.4 Hz, 4H), 2.63 (t, J=7.4 Hz, 4H), 1.98 (p, J=7.3 Hz, 4H). Two exchangeable protons not observed.

### Example 22: N-(1,2,3,5,6,7-hexahydro-s-indacen-4-yl)-5-((1-isopropylazetidin-3-yl)sulfonyl)-4H-1,2,4-triazol-3-amine

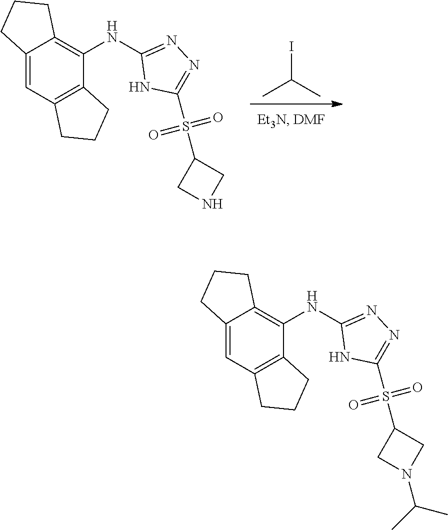

To a solution of 5-(azetidin-3-ylsulfonyl)-N-(1,2,3,5,6,7-hexahydro-s-indacen-4-yl)-4H-1,2,4-triazol-3-amine (Example 21) (100 mg, 211.21 μmol, 1 eq, TFA salt) in DMF (0.5 mL) was added TEA (107 mg, 1.06 mmol, 5 eq) and 2-iodopropane (72 mg, 422.41 μmol, 2 eq). The reaction mixture was stirred at 10° C. for 12 hours. The reaction mixture was filtered and the filtrate was purified by prep-HPLC (column: Phenomenex Gemini C18, 150 mm*25 mm*10 μm; mobile phase: [A: water (0.04% NH₃.H₂O+10 mM NH₄HCO₃); B: MeCN]; B %: 3%-61%, 10 min) and then further purified by prep-HPLC (column: Phenomenex Gemini C18, 150 mm*25 mm*10 μm; mobile phase: [A: water (0.05% NH₄HCO₃); B: MeCN]; B %: 12%-42%,11.5 min) to give the title compound (9.21 mg, 10.74% yield, 98.9% purity on LCMS) as an off-white solid.

¹H NMR (400 MHz, DMSO-d₆): δ 13-15 (br s, 1H), 8.95 (s, 1H), 6.97 (s, 1H), 4.29-4.26 (m, 1H), 3.47-3.45 (m, 2H), 3.35-3.33 (m, 2H), 2.83 (t, 4H), 2.64 (t, 4H), 2.28-2.25 (m, 1H), 2.00-1.96 (m, 4H) and 0.81 (d, 6H).

LCMS: m/z 402.2 (M+H)⁺ (ES⁺).

### Example 23: 5-((3-(diethylamino)propyl)thio)-N-(1,2,3,5,6,7-hexahydro-s-indacen-4-yl)-4H-1,2,4-triazol-3-amine

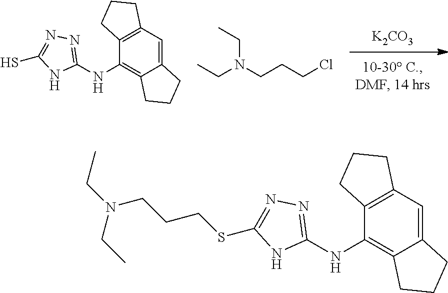

To a solution of 5-((1,2,3,5,6,7-hexahydro-s-indacen-4-yl)amino)-4H-1,2,4-triazole-3-thiol (Intermediate R1) (100 mg, 367.15 μmol, 1 eq) in DMF (2 mL) was added K₂CO₃ (61 mg, 440.58 μmol, 1.2 eq) and 3-chloro-N,N-diethyl-propan-1-amine (82 mg, 550.73 μmol, 1.5 eq). The reaction mixture was stirred at 10° C. for 12 hours. The reaction mixture was warmed to 30° C. and stirred for 2 hours. The reaction mixture was quenched with water (30 mL) and the resulting mixture was stirred for another 1 hour. The mixture was filtered and the filter cake was dried in vacuum to give the title compound (100 mg, 70.64% yield) as a white solid.

¹H NMR (400 MHz, DMSO-d₆): δ 12.33 (br s, 1H), 8.32 (s, 1H), 6.88 (br s, 1H), 2.98 (t, 2H), 2.81 (m, 4H), 2.63 (t, 4H), 2.45-2.41 (m, 6H), 1.98-1.94 (m, 4H), 1.74-1.71 (m, 2H) and 0.93 (t, 6H).

LCMS: m/z 386.2 (M+H)⁺ (ES⁺).

### Example 24: 5-((3-(diethylamino)propyl)sulfonyl)-N-(1,2,3,5,6,7-hexahydro-s-indacen-4-yl)-4H-1,2,4-triazol-3-amine

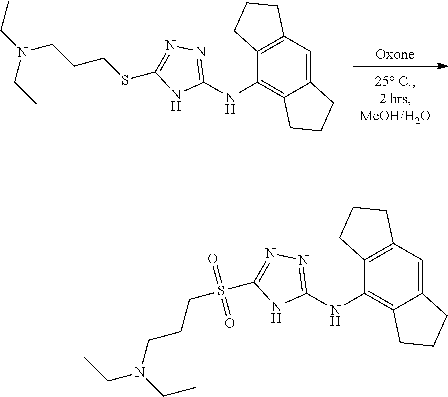 [SMILES: CCN(CC)CCCSc1nnc(Nc2c3c(cc4c2CCC4)CCC3)[nH]1 ; "Oxone" ; CCN(CC)CCCS(=O)(=O)c1nnc(Nc2c3c(cc4c2CCC4)CCC3)[nH]1]

To a solution of 5-((3-(diethylamino)propyl)thio)-N-(1,2,3,5,6,7-hexahydro-s-indacen-4-yl)-4H-1,2,4-triazol-3-amine (Example 23) (90 mg, 233.42 μmol, 1 eq) in MeOH (2.5 mL) and H₂O (2 mL) was added Oxone (430 mg, 700.27 μmol, 3 eq). The reaction mixture was stirred at 25° C. for 2 hours. The reaction mixture was filtered and the filtrate was adjusted with solid NaHCO₃ to pH 5. The solution was purified by prep-HPLC (column: Phenomenex Synergi C18, 150 mm*25 mm*10 μm; mobile phase: [A: water (0.1% TFA); B: MeCN]; B %: 20%-50%, 11 min) to give the title compound (9.55 mg, 7.70% yield, 100% purity on LCMS, TFA salt) as a brown solid.

¹H NMR (400 MHz, DMSO-d₆): δ13.35 (s, 1H), 9.27 (s, 1H), 9.06 (s, 1H), 6.98 (s, 1H), 3.51-348 (m, 2H), 3.11-305 (m, 6H), 2.83 (t, 4H), 2.64 (t, 4H), 2.01-1.96 (m, 6H) and 1.17 (t, 6H).

LCMS: m/z 418.2 (M+H)⁺ (ES⁺).

### Example 24: N-(1,2,3,5,6,7-hexahydro-s-indacen-4-yl)-5-((1-isopropyl-5-(3-methoxyoxetan-3-yl)-1H-pyrazol-3-yl)sulfinyl)-4H-1,2,4-triazol-3-amine

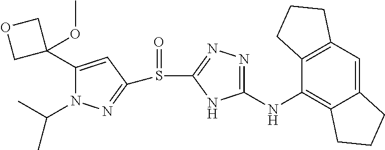 [SMILES: COC1(c2cc(S(=O)c3nnc(Nc4c5c(cc6c4CCC6)CCC5)[nH]3)nn2C(C)C)COC1]

The compound of example 25 was synthesised by a method analogous to those outlined above.

### Example 27: 3-(4-fluoro-2,6-diisopropylbenzyl)-5-((4-fluorobenzyl)thio)-4H-1,2,4-triazole

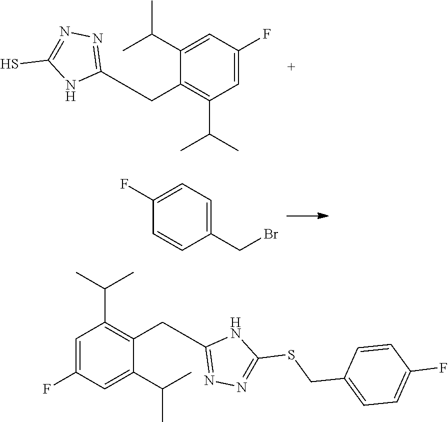

To a solution of 5-(4-fluoro-2,6-diisopropylbenzyl)-4H-1,2,4-triazole-3-thiol (Intermediate R2) (100 mg, 340.83 μmol, 1 eq) in DMF (3 mL) was added TEA (69 mg, 681.66 μmol, 2 eq) and 1-(bromomethyl)-4-fluorobenzene (64 mg, 340.83 μmol, 1 eq). The reaction mixture was stirred at 25° C. for 1 hour. The reaction mixture was diluted with H₂O (20 mL) and extracted with EtOAc (3×30 mL). The combined organic layers were washed with brine (2×20 mL), dried over Na₂SO₄, filtered and concentrated under reduced pressure to give the title compound (100 mg, crude) as a yellow oil.

¹H NMR (400 MHz, DMSO-d₆): δ 7.31-7.26 (m, 2H), 7.03-7.00 (m, 2H), 6.98-6.92 (m, 2H), 4.24 (s, 2H), 4.08 (s, 2H), 3.29-3.24 (m, 2H) and 1.09 (d, 12H). One exchangeable proton not observed.

### Example 28: 3-(4-fluoro-2,6-diisopropylbenzyl)-5-((4-fluorobenzyl)sulfonyl)-4H-1,2,4-triazole

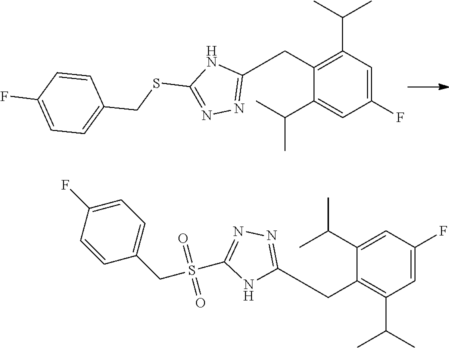 [SMILES: CC(C)c1cc(F)cc(C(C)C)c1Cc1nnc(SCc2ccc(F)cc2)[nH]1 ; CC(C)c1cc(F)cc(C(C)C)c1Cc1nnc(S(=O)(=O)Cc2ccc(F)cc2)[nH]1]

To a solution of 3-(4-fluoro-2,6-diisopropylbenzyl)-5-((4-fluorobenzyl)thio)-4H-1,2,4-triazole (Example 27) (50 mg, 124.53 μmol, 1 eq) in DCM (1 mL) was added m-CPBA (50 mg, 249.06 μmol, 85 wt. % in H₂O, 2 eq). The reaction mixture was stirred at 25° C. for 1 hour. The reaction mixture was concentrated in vacuum. The residue was purified by prep-HPLC (column: Phenomenex Synergi C18, 150 mm*25 mm*10 μm; mobile phase: [A: water (0.1% TFA); B: MeCN]; B %: 50%-80%, 10 min) to give the title compound (2.32 mg, 3.14% yield over two steps, 100% purity on LCMS) as a white solid.

¹H NMR (400 MHz, DMSO-d₆): δ 7.21-7.16 (m, 2H), 7.07 (t, 2H), 6.99 (d, 2H), 4.72 (s, 2H), 4.24 (s, 2H), 3.17-3.13 (m, 2H) and 1.10 (d, 12H). One exchangeable proton not observed.

LCMS: m/z 434.2 (M+H)⁺ (ES⁺).

### Example 20: 3-((1,2,3,5,6,7-hexahydro-s-indacen-4-yl)methyl)-5-((1-isopropyl-1H-pyrazol-3-yl)thio)-4H-1,2,4-triazole

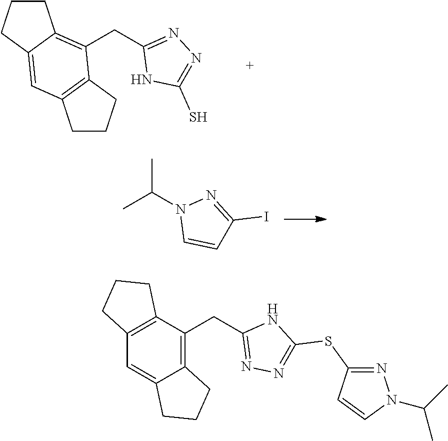

To a solution of 3-iodo-1-isopropyl-H-pyrazole (Intermediate L8) (100 mg, 423.63 μmol, 1 eq) in dioxane (3 mL) were added CuI (161 mg, 847.27 μmol, 2 eq), N¹,N²-dimethylethane-1,2-diamine (746 mg, 8.47 mmol, 20 eq) and 5-((1,2,3,5,6,7-hexahydro-s-indacen-4-yl)methyl)-4H-1,2,4-triazole-3-thiol (Intermediate R3) (191 mg, 423.63 μmol, 1 eq). The reaction mixture was stirred at 100° C. for 12 hours. The reaction mixture was concentrated in vacuum. The residue was purified by reversed phase flash chromatography (0.1% TFA in water-MeCN) to give the title compound (100 mg, 56.60% yield, 91% purity on LCMS) as a yellow solid.

LCMS: m/z 380.1 (M+H)⁺ (ES⁺).

### Example 30: 3-((1,2,3,5,6,7-hexahydro-s-indacen-4-yl)methyl)-5-((1-isopropyl-1H-pyrazol-3-yl)sulfonyl)-4H-1,2,4-triazole

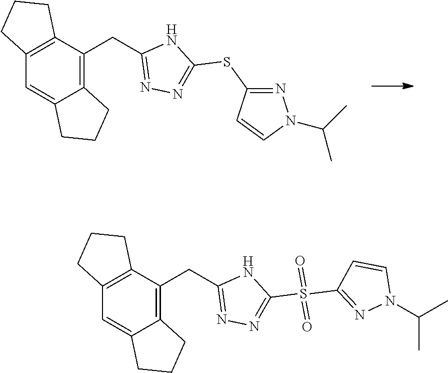 [SMILES: CC(C)n1ccc(Sc2nnc(Cc3c4c(cc5c3CCC5)CCC4)[nH]2)n1 ; CC(C)n1ccc(S(=O)(=O)c2nnc(Cc3c4c(cc5c3CCC5)CCC4)[nH]2)n1]

To a solution of 3-((1,2,3,5,6,7-hexahydro-s-indacen-4-yl)methyl)-5-((1-isopropyl-1H-pyrazol-3-yl)thio)-4H-1,2,4-triazole (Example 29) (20 mg, 52.70 μmol, 1 eq) in MeOH (0.5 mL) and H₂O (0.5 mL) was added Oxone (97 mg, 158.09 μmol, 3 eq), then the reaction mixture was stirred at 25° C. for 16 hours. The reaction mixture was quenched with saturated aqueous NH₄Cl solution (10 mL) and extracted with EtOAc (3×20 mL). The organic layers were dried over anhydrous Na₂SO₄, filtered and concentrated in vacuum. The residue was purified by prep-HPLC (column: Phenomenex Synergi C18, 150 mm*25 mm*10 μm; mobile phase: [A: water (0.1% TFA); B: MeCN]; B %: 45%-75%, 9 min) to give the title compound (5.13 mg, 23.67% yield, 100% purity on LCMS) as a white solid.

¹H NMR (400 MHz, CDCl₃): δ 7.45 (d, 1H), 7.01 (s, 1H), 6.89 (d, 1H), 4.52-4.49 (m, 1H), 4.09 (s, 2H), 2.81 (t, 4H), 2.62 (t, 4H), 2.01-1.93 (m, 4H), 1.43 (d, 6H). One exchangeable proton not observed.

LCMS: m/z 412.2 (M+H)⁺ (ES⁺).

### Example 31:4-(4-((5-((1-isopropyl-1H-pyrazol-3-yl)thio)-4H-1,2,4-triazol-3-yl)methyl)-2,3-dihydro-1H-inden-5-yl)-2-methoxypyridine

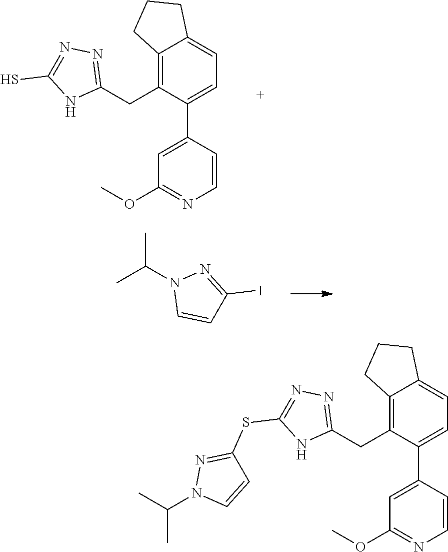

To a solution of 5-((5-(2-methoxypyridin-4-yl)-2,3-dihydro-1H-inden-4-yl)methyl)-4H-1,2,4-triazole-3-thiol (Intermediate R5) (100 mg, 295.49 μmol, 1 eq) in dioxane (4 mL) were added CuI (112 mg, 590.98 μmol, 2 eq), N¹,N²-dimethylethane-1,2-diamine (520 mg, 5.91 mmol, 20 eq) and 3-iodo-1-isopropyl-1H-pyrazole (Intermediate L8) (70 mg, 295.49 μmol, 1 eq). The reaction mixture was stirred at 100° C. for 12 hours. The reaction mixture was concentrated under reduced pressure. The residue was diluted with H₂O (10 mL) and extracted with EtOAc (3×20 mL). The combined organic layers were washed with brine (2×10 mL), dried over Na₂SO₄, filtered and concentrated under reduced pressure. The residue was purified by column chromatography (SiO₂, petroleum ether:ethyl acetate 20:1 to 1:1) to give the title compound (40 mg, 30.31% yield) as a yellow oil.

¹H NMR (400 MHz, DMSO-d₆): δ 13.80 (s, 1H), 8.14 (d, 1H), 7.80 (s, 1H), 7.23 (d, 1H), 7.04-7.00 (m, 1H), 6.95 (d, 1H), 6.78 (s, 1H), 6.29 (d, 1H), 4.48-4.44 (m, 1H), 3.89 (s, 2H), 3.86 (s, 3H), 2.90 (t, 2H), 2.67 (t, 2H), 2.01-1.97 (m, 2H) and 1.39 (d, 6H).

LCMS: m/z 447.3 (M+H)⁺ (ES⁺).

### Example 32: 4-(4-((5-((1-isopropyl-H-pyrazol-3-yl)sulfonyl)-4H-1,2,4-triazol-3-yl)methyl)-2,3-dihydro-1H-inden-5-yl)-2-methoxypyridine

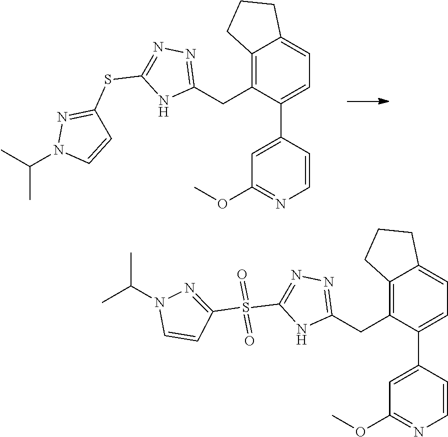 [SMILES: COc1cc(-c2ccc3c(c2Cc2nnc(Sc4ccn(C(C)C)n4)[nH]2)CCC3)ccn1 ; COc1cc(-c2ccc3c(c2Cc2nnc(S(=O)(=O)c4ccn(C(C)C)n4)[nH]2)CCC3)ccn1]

To a solution of 4-(4-((5-((1-isopropyl-H-pyrazol-3-yl)thio)-4H-1,2,4-triazol-3-yl)methyl)-2,3-dihydro-1H-inden-5-yl)-2-methoxypyridine (Example 31) (15 mg, 33.59 μmol, 1 eq) in DCM (1 mL) was added m-CPBA (13 mg, 67.18 μmol, 85 wt. % in H₂O, 2 eq). The mixture was stirred at 25° C. for 2 hours. The reaction mixture was concentrated in vacuum. The residue was purified by prep-HPLC (Waters Xbridge C18, 150 mm*25*mm*5 μm; mobile phase: [A: water (0.05% ammonia hydroxide v/v), B: MeCN]; B %: 5%-35%, 10 min) to give the title compound (1.32 mg, 8.21% yield, 100% purity on LCMS) as a white solid.

¹H NMR (400 MHz, DMSO-d₆): δ 8.07 (d, 1H), 8.03 (d, 1H), 7.23 (d, 1H), 7.04 (d, 1H), 6.86 (d, 1H), 6.76 (s, 2H), 4.63-4.56 (m, 1H), 3.96 (s, 2H), 3.83 (s, 3H), 2.89 (t, 2H), 2.61 (t, 2H), 2.01-1.93 (m, 2H) and 1.40 (d, 6H). One exchangeable proton not observed.

LCMS: m/z 479.2 (M+H)⁺ (ES⁺).

### Example 3s: 5-((1-isopropyl-H-pyrazol-3-yl)thio)-N-(5-(2-methoxypyridin-4-yl)-2,3-dihydro-1H-inden-4-yl)-4H-1,2,4-triazol-3-amine

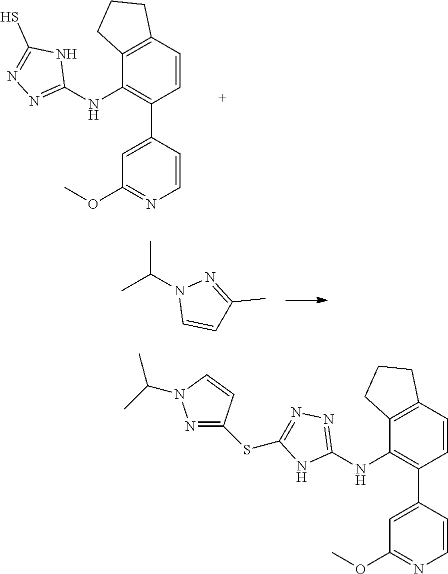

To a solution of 5-((5-(2-methoxypyridin-4-yl)-2,3-dihydro-1H-inden-4-yl)amino)-4H-1,2,4-triazole-3-thiol (Intermediate R6) (0.1 g, 294.63 μmol, 1 eq) and 3-iodo-1-isopropyl-1H-pyrazole (Intermediate L8) (62 mg, 265.16 μmol, 0.9 eq) in dioxane (5 mL) were added CuI (112 mg, 589.25 μmol, 2 eq) and N¹,N²-dimethylethane-1,2-diamine (519 mg, 5.89 mmol, 20 eq) under N₂. The reaction mixture was stirred at 70° C. for 1 hour. The mixture was poured into water (30 mL) and extracted with EtOAc (2×30 mL). The combined organic phases were washed with brine (2×50 mL), dried over anhydrous Na₂SO₄ filtered and concentrated in vacuum. The residue was purified by column chromatography (SiO₂, petroleum ether:ethyl acetate 4:1 to 1:1) to give the title compound (0.1 g, 69.77% yield, 92% purity on LCMS) as a brown solid.

¹H NMR (400 MHz, DMSO-d₆): δ 12.26 (s, 1H), 8.54 (s, 1H), 8.10 (d, 1H), 7.76 (s, 1H), 7.23-7.14 (m, 2H), 6.91 (d, 1H), 6.72 (s, 1H), 6.21 (s, 1H), 4.49-4.42 (m, 1H), 3.83 (s, 3H), 2.92 (t, 2H), 2.66 (t, 2H), 2.01-1.96 (m, 2H) and 1.38 (d, 6H).

LCMS: m/z 448.2 (M+H)⁺ (ES⁺).

### Example 34:5-((1-isopropyl-1H-pyrazol-3-yl)sulfonyl)-N-(5-(2-methoxypyridin-4-yl)-2,3-dihydro-1H-inden-4-yl)-4H-1,2,4-triazol-3-amine

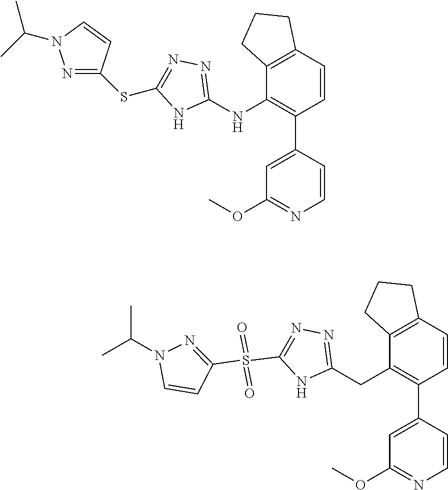 [SMILES: COc1cc(-c2ccc3c(c2Nc2nnc(Sc4ccn(C(C)C)n4)[nH]2)CCC3)ccn1 ; COc1cc(-c2ccc3c(c2Cc2nnc(S(=O)(=O)c4ccn(C(C)C)n4)[nH]2)CCC3)ccn1]

To a solution of 5-((1-isopropyl-1H-pyrazol-3-yl)thio)-N-(5-(2-methoxypyridin-4-yl)-2,3-dihydro-1H-inden-4-yl)-4H-1,2,4-triazol-3-amine (Example 33) (0.22 g, 491-56 μmol, 1 eq) in MeOH (3 mL) and H₂O (3 mL) was added Oxone (604 mg, 983.12 μmol, 2 eq). Then the reaction mixture was stirred at 25° C. for 16 hours. Then to the above mixture was added Oxone (604 mg, 983.12 μmol, 2 eq), and the reaction mixture was stirred at 25° C. for 24 hours. The reaction mixture was purified by reversed phase flash chromatography (0.1% TFA in water-MeCN) to give 200 mg of impure product. The impure product was dissolved into THF (1 mL). To the solution was added 4,4,4′,4′,5,5,5′,5′-octamethyl-2,2′-bi(1,3,2-dioxaborolane) (500 mg) and the resulting mixture was stirred at 25° C. for 2 hours. The mixture was concentrated in vacuum. The residue was purified by prep-HPLC (column: Phenomenex Synergi C18, 150 mm*25 mm*10 μm; mobile phase: [A: water (0.1% TFA); B: MeCN]; B %: 30%-54%, 8 min) to give the title compound (100.34 mg, 3439% yield, 100% purity on LCMS, TFA salt) as a white solid.

¹H NMR (400 MHz, DMSO-d₆): δ 13.09 (br s, 1H), 8.96 (s, 1H), 8.05 (dd, 2H), 7.24 (t, 1H), 7.17 (d, 1H), 6.86 (dd, 1H), 6.74 (d, 1H), 6.69 (s, 1H), 4.65-4.58 (m, 1H), 3.81 (s, 3H), 2.93 (t, 2H), 2.57 (t, 2H), 2.01-1.94 (m, 2H) and 1.42 (d, 6H).

LCMS: m/z 480.1 (M+H)⁺ (ES⁺).

### Example 34: N-(7-fluoro-5-(pyridin-4-yl)-2,3-dihydro-1H-inden-4-yl)-5-((1-isopropyl-1H-pyrazol-3-yl)thio)-4H-1,2,4-triazol-3-amine

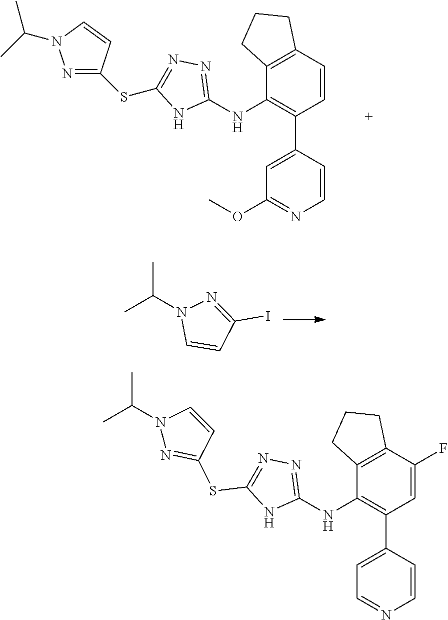

To a mixture of 5-((7-fluoro-5-(pyridin-4-yl)-2,3-dihydro-1H-inden-4-yl)amino)-4H-1,2,4-triazole-3-thiol (Intermediate R8) (130 mg, 397.09 μmol, 1 eq) and 3-iodo-1-isopropyl-1H-pyrazole (Intermediate L8) (89 mg, 377.24 μmol, 0.95 eq) in dioxane (5 mL) were added CuI (151 mg, 794.19 μmol, 2 eq) and N¹,N²-dimethylethane-1,2-diamine (700 mg, 7.94 mmol, 20 eq) in one portion under N₂. Then the reaction mixture was heated to 70° C. and stirred for 0.5 hours. The mixture was filtered and the filtrate was concentrated in vacuum. The residue was purified by column chromatography (SiO₂, petroleum ether:ethyl acetate 10:1 to 1:1 and then ethanol:ethyl acetate 20:1 to 10:1) to give the title compound (120 mg, 55% yield, 79.38% purity on LCMS) as a yellow oil.

¹H NMR (400 MHz, CDCl₃): δ 8.59 (d, 2H), 7.49 (d, 1H), 7.27 (d, 2H), 6.87 (d, 1H), 6.38 (d, 1H), 5.70 (br s, 1H), 4.54-4.49 (m, 1H), 2.90 (t, 2H), 2.82-2.79 (m, 2H), 2.20-2.15 (m, 2H) and 1.54 (d, 6H). One exchangeable proton not observed.

LCMS: m/z 436.2 (M+H)⁺ (ES⁺).

### Example 36: N-(7-fluoro-5-(pyridin-4-yl)-2,3-dihydro-1H-inden-4-yl)-5-((1-isopropyl-1H-pyrazol-3-yl)sulfonyl)-4H-1,2,4-triazol-3-amine

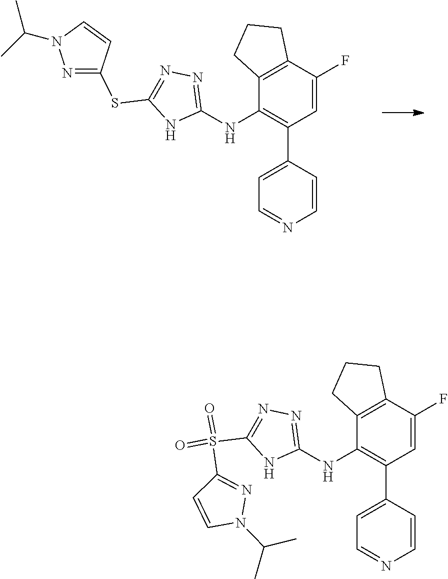 [SMILES: CC(C)n1ccc(Sc2nnc(Nc3c(-c4ccncc4)cc(F)c4c3CCC4)[nH]2)n1 ; CC(C)n1ccc(S(=O)(=O)c2nnc(Nc3c(-c4ccncc4)cc(F)c4c3CCC4)[nH]2)n1]

To a mixture of N-(7-fluoro-5-(pyridin-4-yl)-2,3-dihydro-1H-inden-4-yl)-5-((1-isopropyl-1H-pyrazol-3-yl)thio)-4H-1,2,4-triazol-3-amine (Example 35) (100 mg, 229.61 μmol, 1 eq) in MeOH (5 mL) and H₂O (5 mL) was added Oxone (282 mg, 459.22 μmol, 2 eq) in one portion at 25° C. Then the reaction mixture was stirred at 25° C. for 26 hours. Oxone (282 mg, 459.22 μmol, 2 eq) was added to the above mixture and the resulting reaction mixture was continued for 24 hours. Finally 4,4,4′,4′,5,5,5′,5′-octamethyl-2,2′-bi(1,3,2-dioxaborolane) (291 mg, 1.15 mmol, s eq) was added to the reaction mixture and the reaction mixture was stirred at 25° C. for 1 hour. The mixture was concentrated in vacuum. The residue was purified by reversed phase flash chromatography (0.1% TFA in water-MeCN) and then further purified by prep-HPLC (column: Waters Xbridge C18, 150 mm*25 mm*5 μm; mobile phase: [A: water (0.05% ammonia hydroxide v/v); B: MeCN]; B %: 3%-33%, 10 min) to give the title compound (13.87 mg, 12% yield, 100% purity on LCMS) as a white solid.

¹H NMR (400 MHz, DMSO-d₆): δ 8.78 (br s, 1H), 8.48 (dd, 2H), 8.02 (d, 1H), 7.28 (d, 2H), 7.07 (d, 1H), 6.71 (d, 1H), 4.64-4.57 (m, 1H), 2.96 (t, 2H), 2.65 (t, 2H), 2.06-2.02 (m, 2H) and 1.41 (d, 6H). One exchangeable proton not observed.

LCMS: m/z 468.2 (M+H)⁺ (ES⁺).

### Example 37: N-(4-fluoro-2-isopropyl-6-(pyridin-3-yl)phenyl)-5-((1-isopropyl-1H-pyrazol-3-yl)thio)-4H-1,2,4-triazol-3-amine

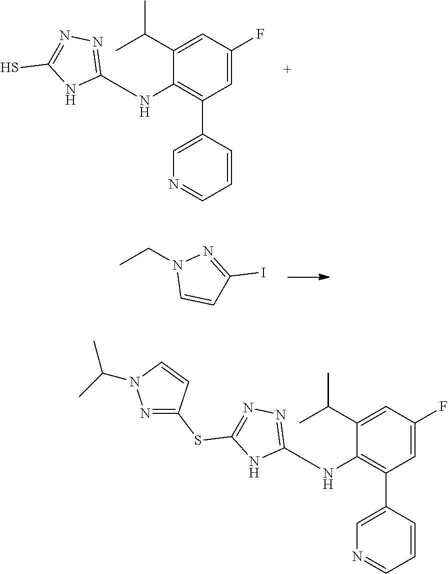

To a solution of 5-((4-fluoro-2-isopropyl-6-(pyridin-3-yl)phenyl)amino)-4H-1,2,4-triazole-3-thiol (Intermediate R10) (10 mg, 30.36 μmol, 1 eq) in dioxane (1 mL) were added 3-iodo-1-isopropyl-1H-pyrazole (Intermediate L8) (6 mg, 27.32 μmol, 0.9 eq), N¹,N²-dimethylethane-1,2-diamine (53 mg, 607.17 μmol, 20 eq) and CuI (n mg, 60.72 μmol, 2 eq). Then the mixture was stirred at 5° C. for 5 minutes. Then the mixture was stirred at 25° C. for 5 minutes. The reaction mixture was partitioned between H₂O (10 mL) and EtOAc (10 mL). The organic phase was separated, washed with brine (10 mL), dried over anhydrous Na₂SO₄, filtered and concentrated under reduced pressure to give the title compound (12 mg, crude) as a yellow oil.

LCMS: m/z 438.1 (M+H)⁺ (ES⁺).

### Example 38: N-(4-fluoro-2-isopropyl-6-(pyridin-3-yl)phenyl)-5-((1-isopropyl-1H-pyrazol-3-yl)sulfinyl)-4H-1,2,4-triazol-3-amine

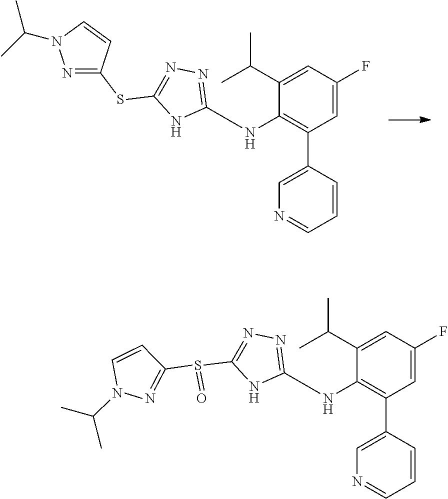 [SMILES: CC(C)c1cc(F)cc(-c2cccnc2)c1Nc1nnc(Sc2ccn(C(C)C)n2)[nH]1 ; CC(C)c1cc(F)cc(-c2cccnc2)c1Nc1nnc(S(=O)c2ccn(C(C)C)n2)[nH]1]

To a solution of N-(4-fluoro-2-isopropyl-6-(pyridin-3-yl)phenyl)-5-((1-isopropyl-1H-pyrazol-3-yl)thio)-4H-1,2,4-triazol-3-amine (Example 37) (10 mg, 22.86 μmol, 1 eq) in MeOH (0.5 mL) and H₂O (0.5 mL) was added Oxone (28 mg, 45.71 μmol, 2 eq). Then the reaction mixture was stirred at 25° C. for 50 hours. The mixture was quenched with H₂O (10 mL) and extracted with EtOAc (2×20 mL). The combined organic layers were dried over anhydrous Na₂SO₄, filtered and concentrated in vacuum. The residue was purified by prep-HPLC (column: Luna C18, 150 mm*25 mm*5 μm; mobile phase: [A: water (0.1% TFA); B: MeCN]; B %: 20%-50%, 10 min) to give the title compound (2 mg, 20.8% yield over two steps, 81% purity on LCMS) as a yellow oil.

LCMS: m/z 454.2 (M+H)⁺ (ES⁺).

### Example 39: N-(4-fluoro-2-isopropyl-6-(pyridin-3-yl)phenyl)-5-((1-isopropyl-1H-pyrazol-3-yl)sulfonyl)-4H-1,2,4-triazol-3-amine

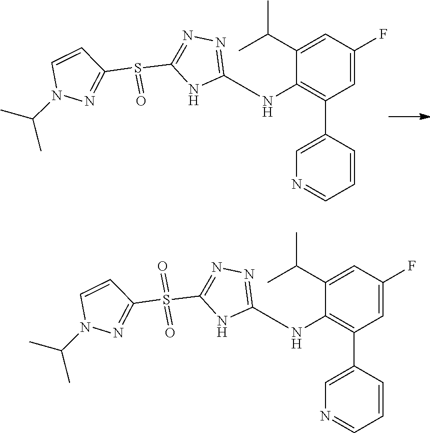

To a solution of N-(4-fluoro-2-isopropyl-6-(pyridin-3-yl)phenyl)-5-((1-isopropyl-1H-pyrazol-3-yl)sulfinyl)-4H-1,2,4-triazol-3-amine (2 mg, 3.57 μmol, 1 eq) in MeOH (0.5 mL) and H₂O (0.5 mL) was added Oxone (4 mg, 7.14 μmol, 2 eq). Then the reaction mixture was stirred at 25° C. for 13 hours. To the mixture was added 4,4,4′,4′,5,5,5′,5′-octamethyl-2,2′-bi(1,3,2-dioxaborolane) (2 mg, 10.72 μmol, 3 eq), and the mixture was stirred at 25° C. for 40 minutes. The mixture was quenched with H₂O (10 mL) and extracted with EtOAc (2×20 mL). The organic layers were dried over anhydrous Na₂SO₄, filtered and concentrated in vacuum. The residue was purified by prep-HPLC (column: Phenomenex Synergi C18, 150 mm*25 mm*5 μm; mobile phase: [A: water (0.1% TFA); B: MeCN]; B %: 18%-48%, 10 minutes) to give the title compound (520 μg, 30.38% yield, 98% purity on LCMS) as a white solid.

¹H NMR (400 MHz, CD₃OD): δ 8.51 (s, 1H), 8.40 (s, 1H), 7.85-7.80 (m, 2H), 7.35 (dd, 1H), 7.24 (dd, 1H), 7.05 (dd, 1H), 6.75 (d, 1H), 4.65-4.58 (m, 1H), 3.23-3.20 (m, 1H), 1.50 (d, 6H) and 1.19 (d, 6H). Two exchangeable protons not observed.

LCMS: m/z 470.2 (M+H)⁺ (ES⁺).

### Example 40: N-(1,2,3,5,6,7-hexahydro-s-indacen-4-yl)-5-(phenylsulfonyl)-1H-1,2,4-triazol-3-amine

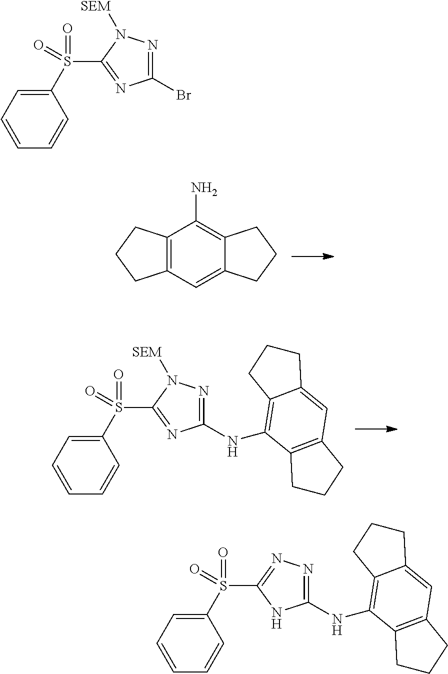 [SMILES: C[Si](C)(C)CCOCn1nc(Br)nc1S(=O)(=O)c1ccccc1 ; Nc1c2c(cc3c1CCC3)CCC2 ; C[Si](C)(C)CCOCn1nc(Nc2c3c(cc4c2CCC4)CCC3)nc1S(=O)(=O)c1ccccc1 ; O=S(=O)(c1ccccc1)c1nnc(Nc2c3c(cc4c2CCC4)CCC3)[nH]1]

Step A: An oven dried vial was charged with 3-bromo-5-(phenylsulfonyl)-1-((2-(trimethylsilyl)ethoxy)methyl)-1H-1,2,4-triazole (Intermediate A1) (250 mg, 0.60 mmol), K₂CO₃ (132 mg, 0.96 mmol), Pd-175 (46.6 mg, 0.060 mmol),1,2,3,5,6,7-hexahydro-s-indacen-4-amine (114 mg, 0.66 mmol) and dioxane (4 mL). The reaction was heated to 60° C. overnight. The reaction was diluted with EtOAc (30 mL), washed with water (30 mL) and sat aq NH₄Cl (30 mL). The organic phase was separated, dried (phase separator) and concentrated in vacuo. The product was purified by chromatography on silica gel (12 g column, 0-100% EtOAc/isohexane) to afford N-(1,2,3,5,6,7-hexahydro-s-indacen-4-yl)-5-(phenylsulfonyl)-1-((2-(trimethylsilyl)-ethoxy)methyl)-1H-1,2,4-triazol-3-amine (136 mg, 42% yield) as a pale white solid.

LCMS m/z 511.2 (M+H)⁺ (ES⁺).

¹H NMR (DMSO-d₆) δ 8.49 (s, 1H), 8.05 (d, J=7.3 Hz, 2H), 7.86 (t, J=7.5 Hz, 1H), 7.74 (t, J=7.9 Hz, 2H), 6.86 (s, 1H), 5.66 (s, 2H), 3.54-3.51 (m, 2H), 2.77 (t, J=7.3 Hz, 4H), 1.88 (p, J=7.4 Hz, 4H), 0.84-0.76 (m, 2H), −0.06 (s, 9H), 2×CH₂ obscured by DMSO peak.

Step B: N-(1,2,3,5,6,7-hexahydro-s-indacen-4-yl)-5-(phenylsulfonyl)-1-((2-(trimethylsilyl)ethoxy)methyl)-1H-1,2,4-triazol-3-amine (50 mg, 0.098 mmol) was dissolved in DCM (2 mL) and TFA (1 mL) was added. The reaction was stirred at RT for min before being concentrated in vacuo. The crude product was purified by basic prep HPLC (5-50% MeCN in water) to afford the title compound (14 mg, 36% yield) as a pale white solid.

LCMS m/z 381.0 (M+H)⁺ (ES⁺).

¹H NMR (DMSO-d₆) δ 13.14 (s, 1H), 8.97 (s, 1H), 7.93 (d, J=7.4 Hz, 2H), 7.77 (t, J=7.5 Hz, 1H), 7.67 (t, J=7.8 Hz, 2H), 6.95 (s, 1H), 2.80 (t, J=7.3 Hz, 4H), 1.92 (p, J=7.4 Hz, 4H). 2×CH₂ obscured by DMSO peak.

### Example 41: N-(7-fluoro-5-(pyridin-3-yl)-2,3-dihydro-1H-inden-4-yl)-5-(phenylsulfonyl)-1H-1,2,4-triazol-3-amine

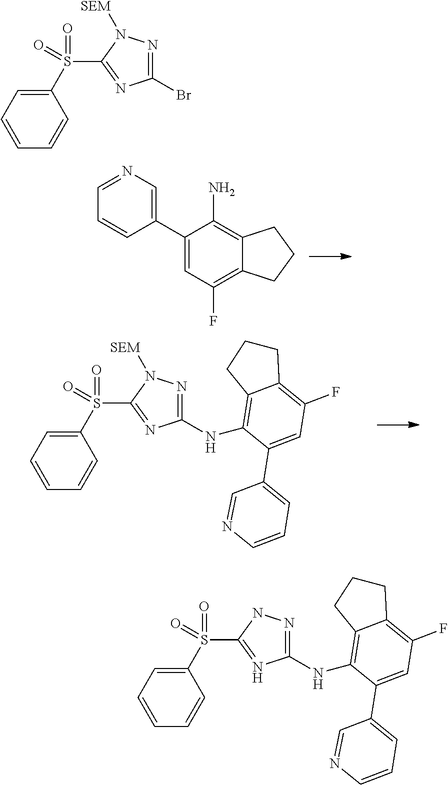

Step A: Prepared according to the general procedure of N-(1,2,3,5,6,7-hexahydro-s-indacen-4-yl)-5-(phenylsulfonyl)-1H-1,2,4-triazol-3-amine (Example 40, Step A) from 3-bromo-5-(phenylsulfonyl)-1-((2-(trimethylsilyl)ethoxy)methyl)-1H-1,2,4-triazole (Intermediate A1) and 7-fluoro-5-(pyridin-3-yl)-2,3-dihydro-1H-inden-4-amine (Intermediate C3) to afford N-(7-fluoro-5-(pyridin-3-yl)-2,3-dihydro-1H-inden-4-yl)-5-(phenylsulfonyl)-1-((2-(trimethylsilyl)ethoxy)methyl)-1H-1,2,4-triazol-3-amine (29.7 mg, 10% yield) as a light brown solid.

Step B: N-(7-fluoro-5-(pyridin-3-yl)-2,3-dihydro-1H-inden-4-yl)-5-(phenylsulfonyl)-1-((2-(trimethylsilyl)ethoxy)methyl)-1H-1,2,4-triazol-3-amine (29.7 mg, 0.047 mmol) was stirred with SCX (300 mg) in MeCN/H₂O for 5 h. The SCX was filtered and washed with MeOH (30 mL), and the product eluted with 0.7 M NH₃ in MeOH (30 mL). The ammoniacal phase was concentrated in vacuo. The crude product was purified by acidic prep HPLC (20-50% MeCN in water) to afford the title compound (3 mg, 1% yield over 2 steps) as a pale white solid.

LCMS m/z 436.1 (M+H)⁺ (ES⁺); 434.0 (M−H)⁻ (ES⁻).

¹H NMR (DMSO-d₆) δ 8.92 (br s, 1H), 8.47 (d, J=2.3 Hz, 1H), 8.41 (dd, J=4.8, 1.7 Hz, 1H), 7.86-7.80 (m, 2H), 7.78-7.73 (m, 1H), 7.69-7.61 (m, 3H), 7.27-7.21 (m, 1H), 7.09 (d, J=9.1 Hz, 1H), 2.97 (t, J=7.5 Hz, 2H), 2.64 (t, J=7.5 Hz, 2H), 2.04 (p, J=7.6 Hz, 2H). One exchangeable proton not observed.

### Example 42: N-(4-fluoro-2,6-diisopropylphenyl)-3-(phenylsulfonyl)-1H-1,2,4-triazol-5-amine

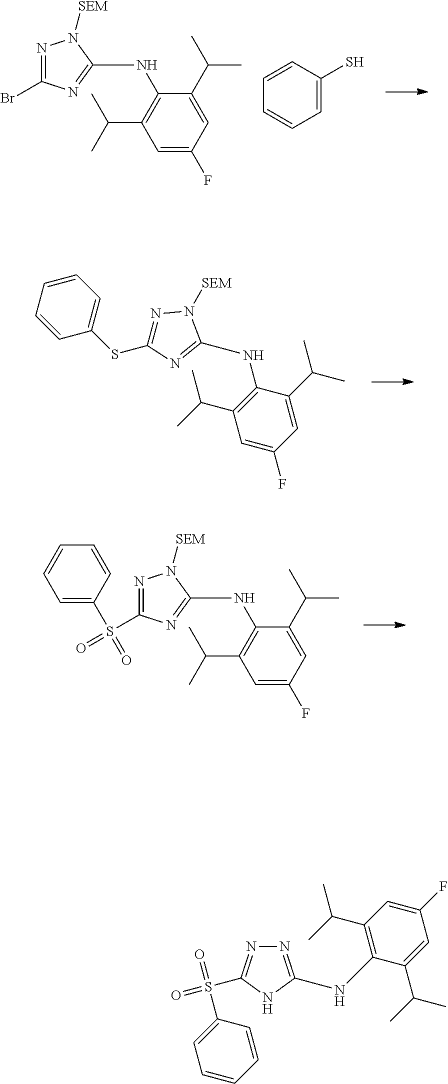

Step A: To 3-bromo-N-(4-fluoro-2,6-diisopropylphenyl)-1-((2-(trimethylsilyl)ethoxy)-methyl)-1H-1,2,4-triazol-5-amine (Intermediate Bi) (1.82 g, 3.86 mmol), benzenethiol (0.436 mL, 4.25 mmol), K₂CO₃ (0.854 g, 6.18 mmol) and Pd-175 (0.302 g, 0.386 mmol) at RT was added 1,4-dioxane (40 mL). The resulting mixture was degassed with N₂ and then stirred at 70° C. overnight. The reaction mixture was filtered through Celite (washing with EtOAc) and the filtrate concentrated in vacuo. The crude product was purified by chromatography on silica gel (80 g column, 0-20% EtOAc/isohexane) to afford a mixture of starting material and product. The mixture was resubmitted to the reaction conditions by addition of benzenethiol (0.43 mL, 4.22 mmol), K₂CO₃ (849 mg, 6.14 mmol), Pd-175 (300 mg, 0.384 mmol) and dioxane (80 mL). The reaction mixture was purged with N₂ and heated to 60° C. overnight. The reaction mixture was filtered through Celite (washing with EtOAc) and the filtrate concentrated in vacuo. The crude product was purified by chromatography on silica gel (40 g column, 0-20% EtOAc/isohexane) to afford N-(4-fluoro-2,6-diisopropylphenyl)-3-(phenylthio)-1-((2-(trimethylsilyl)ethoxy)methyl)-1H-1,2,4-triazol-5-amine (166 mg, 8% yield) as a yellow oil and 1 g of a 1:1 mixture of 3-bromo-N-(4-fluoro-2,6-diisopropylphenyl)-1-((2-(trimethylsilyl)ethoxy)methyl)-1H-1,2,4-triazol-5-amine and N-(4-fluoro-2,6-diisopropylphenyl)-3-(phenylthio)-1-((2-(trimethylsilyl)ethoxy)-methyl)-1H-1,2,4-triazol-5-amine which was used in the next step without further purification.

Step B: To the 1:1 mixture obtained above in DCM (20 mL) was added m-CPBA (0.172 g, 0.999 mmol) at 0° C. The reaction was stirred at this temperature for 10 min before warming to RT. Three additional portions of m-CPBA (0.172 g, 0.999 mmol) were added after 1.5 h, 5 h and 20 h respectively. The reaction was stirred for 4 h and quenched with Na₂SO₃. The solution was extracted with DCM (2×40 mL), the organic extracts were combined, washed with sat aq NaHCO₃ (30 mL), dried (phase separator) and concentrated in vacuo to afford a mixture of N-(4-fluoro-2,6-diisopropylphenyl)-3-(phenylsulfonyl)-1-((2-(trimethylsilyl)ethoxy)methyl)-1H-1,2,4-triazol-5-amine and 3-bromo-N-(4-fluoro-2,6-diisopropylphenyl)-1-((2-(trimethylsilyl)ethoxy)methyl)-1H-1,2,4-triazol-5-amine (57:43, 1.0 g) as a yellow solid.

Step C: The above mixture was dissolved in DCM (4 mL) and TFA (2 mL) and stirred for 5 h. The reaction was concentrated in vacuo. The crude product was purified by chromatography on silica gel (12 g column, 0-50% EtOAc/isohexane) to afford the title compound (40 mg, 9% yield over 2 steps) as a pale white solid.

LCMS m/z 403.2 (M+H)⁺ (ES⁺); 401.1 (M−H)⁻ (ES⁻).

¹H NMR (DMSO-d₆) δ 12.96 (s, 1H), 8.79 (s, 1H), 7.94-7.88 (m, 2H), 7.78-7.72 (m, 1H), 7.65 (t, J=7.8 Hz, 2H), 7.01 (d, J=9.9 Hz, 2H), 2.96 (sept, J=6.8 Hz, 2H), 1.03 (d, J=6.8 Hz, 12H).

### Example 43: 5-((4-((dimethylamino)methyl)phenyl)sulfonyl)-N-(1,2,3,5,6,7-hexahydro-s-indacen-4-yl)-4H-1,2,4-triazol-3-amine

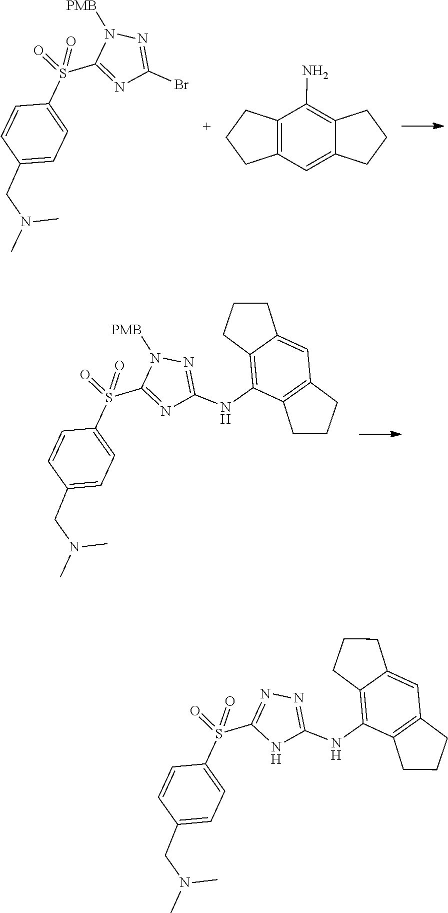

Step A: Pd-175 (69.3 mg, 0.089 mmol), ground K₂CO₃ (131 mg, 0.945 mmol), 1,2,3,5,6,7-hexahydro-s-indacen-4-amine (113 mg, 0.650 mmol) and 1-(4-((3-bromo-1-(4-methoxybenzyl)-1H-1,2,4-triazol-5-yl)sulfonyl)phenyl)-N,N-dimethylmethanamine (Intermediate A2) (275 mg, 0.591 mmol) were charged in a reaction vessel and purged with N₂. 1,4-Dioxane (10 mL) was added, the reaction was degassed with N₂ for min and stirred at 75° C. for 16 h. The reaction mixture was concentrated in vacuo, DCM (2 mL) was added and the mixture loaded onto a column of SCX (10 g). The column was washed with MeOH/DCM (1:1, 50 mL) and the product was eluted with 0.7 M ammonia in MeOH (70 mL). The ammoniacal solution was concentrated in vacuo and the crude product was purified by chromatography on silica gel (40 g column, 0-10% (0.7 M ammonia/MeOH)/DCM) to afford 5-((4-((dimethylamino)methyl)phenyl)-sulfonyl)-N-(1,2,3,5,6,7-hexahydro-s-indacen-4-yl)-1-(4-methoxybenzyl)-1H-1,2,4-triazol-3-amine (160 mg, 39% yield) as a orange oil.

LCMS m/z 558.2 (M+H)⁺ (ES⁺).

¹H NMR (DMSO-d₆) δ 8.43 (s, 1H), 7.91 (d, J=8.5 Hz, 2H), 7.59 (d, J=8.3 Hz, 2H), 7.19 (d, J=8.7 Hz, 2H), 6.91 (d, J=8.7 Hz, 2H), 6.81 (s, 1H), 5.52 (s, 2H), 3.75 (s, 3H), 3.50 (s, 2H), 2.73 (t, J=7.4 Hz, 4H), 2.41 (t, J=7.3 Hz, 4H), 2.16 (s, 6H), 1.82 (p, J=7.4 Hz, 4H).

Step B: 5-((4-((Dimethylamino)methyl)phenyl)sulfonyl)-N-(1,2,3,5,6,7-hexahydro-s-indacen-4-yl)-1-(4-methoxybenzyl)-1H-1,2,4-triazol-3-amine (160 mg, 0.287 mmol) was dissolved in TFA (4 mL) and stirred at 70° C. for 4 h. The solution was concentrated in vacuo and the residue taken up in DCM (30 mL) and washed with water (30 mL). The organic phase was separated, dried (MgSO₄) and concentrated in vacuo. The crude product was purified by acidic prep HPLC (20-50% MeCN in water) to afford the title compound (17 mg, 13% yield) as a white solid.

LCMS m/z 438.3 (M+H)⁺ (ES⁺).

¹H NMR (DMSO-d₆) δ 13.2 (br s, 1H) 8.99 (s, 1H), 7.97 (d, J=8.0 Hz, 2H), 7.69 (d, J=8.0 Hz, 2H), 6.94 (s, 1H), 4.02 (br s, 2H), 2.80 (t, J=7.4 Hz, 4H), 1.92 (p, J=7.4 Hz, 4H). 2×CH₂ obscured by DMSO-d₆ peak. 2×CH₃ obscured by water peak in DMSO-d₆.

### Example 44: 5-((3-((dimethylamino)methyl)phenyl)sulfonyl)-N-(1,2,3,5,6,7-hexahydro-s-indacen-4-yl)-4H-1,2,4-triazol-3-amine

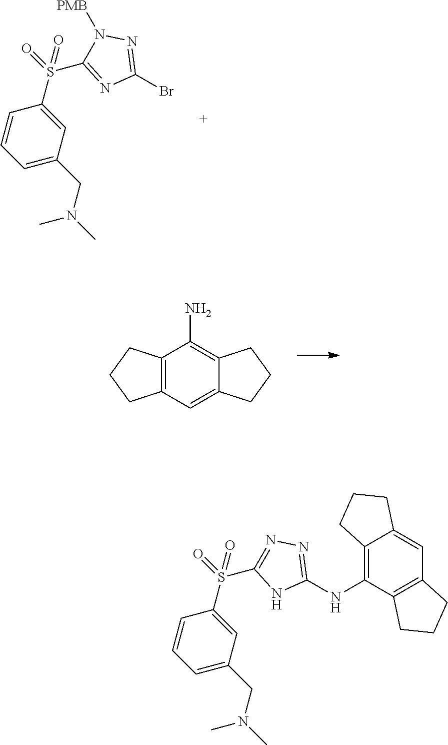

Prepared according to the general procedure of 5-((4-((dimethylamino)methyl)-phenyl)sulfonyl)-N-(1,2,3,5,6,7-hexahydro-s-indacen-4-yl)-4H-1,2,4-triazol-3-amine (Example 43) from 1-(3-((3-bromo-1-(4-methoxybenzyl)-1H-1,2,4-triazol-5-yl)sulfonyl)phenyl)-N,N-dimethylmethanamine (Intermediate A3) to afford the title compound (4 mg, 3% yield) as a white solid.

LCMS m/z 438.3 (M+H)⁺ (ES⁺); 436.3 (M−H)⁻ (ES⁻).

¹H NMR (DMSO-d₆) δ 13.19 (br s, 1H), 8.97 (s, 1H), 8.03 (br s, 1H), 7.96-7.86 (m, 1H), 7.81 (br s, 1H), 7.70 (t, J=7.8 Hz, 1H), 6.94 (s, 1H), 4.04 (br s, 2H), 2.79 (t, J=7.4 Hz, 4H), 1.91 (p, J=7.4 Hz, 4H). 2×CH₂ overlapped with DMSO-d₆ peak, 2×CH₃ overlapped with water peak in DMSO-d₆.

### Example 45: N-(4-fluoro-2-isopropyl-6-(2-methoxypyridin-4-yl)phenyl)-3-(phenylsulfonyl)-1H-1,2,4-triazol-5-amine

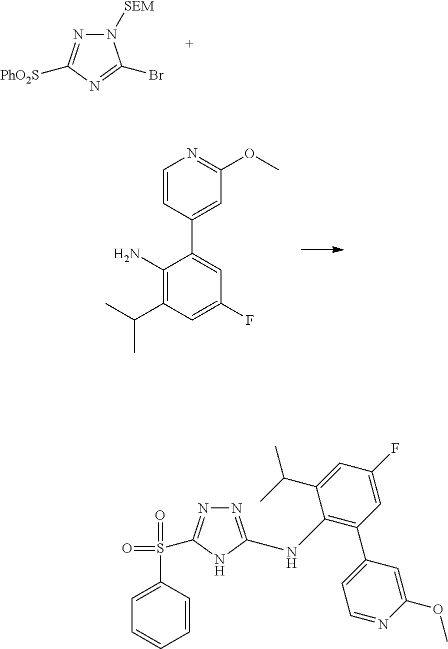

To an ice cooled stirred solution of 5-bromo-3-(phenylsulfonyl)-1-((2-(trimethylsilyl)-ethoxy)methyl)-1H-1,2,4-triazole (Intermediate A4) (110 mg, 0.263 mmol) and 4-fluoro-2-isopropyl-6-(2-methoxypyridin-4-yl)aniline (Intermediate C2) (137 mg, 0.526 mmol) in THF (1 mL) under N₂ was added LiHMDS (1 M in THF, 0.526 mL, 0.526 mmol) dropwise. The reaction was stirred at RT for 2 h and quenched by the slow addition of sat aq NH₄Cl. The mixture was extracted with DCM (2×20 mL) and the combined organic layers were dried (phase separator) and concentrated in vacuo. The product was purified by chromatography on silica gel (12 g column, 0-50% EtOAc/isohexane) to afford N-(4-fluoro-2-isopropyl-6-(2-methoxypyridin-4-yl)phenyl)-3-(phenylsulfonyl)-1-((2-(trimethylsilyl)ethoxy)methyl)-1H-1,2,4-triazol-5-amine (133 mg, 81% yield) as a pale brown solid. The material was then stirred with SCX (1.4 g) in MeCN/H₂O (3:1, 25 mL) for 3 h. The mixture was filtered and the filtrate concentrated in vacuo. The product was purified by basic prep HPLC (35-65% MeCN in water) to afford the title compound (13 mg, 10% yield over 2 steps) as a pale tan solid.

LCMS m/z 468.3 (M+H)⁺ (ES⁺); 466.1 (M−H)⁻ (ES⁻).

¹H NMR (DMSO-d₆) δ 8.86 (br s, 1H), 7.96 (d, J=5.2 Hz, 1H), 7.80 (d, J=7.5 Hz, 2H), 7.76 (t, J=7.4 Hz, 1H), 7.64 (t, J=7.8 Hz, 2H), 7.31 (dd, J=10.0, 2.9 Hz, 1H), 7.10 (dd, J=8.7, 2.9 Hz, 1H), 6.82 (d, J=5.2 Hz, 1H), 6.68 (s, 1H), 3.78 (s, 3H), 3.08-3.01 (m, 1H), 1.08 (d, J=6.8 Hz, 6H). One exchangeable proton not observed.

### Example 46: 5-((2-(diethylamino)ethyl)sulfonyl)-N-(1,2,3,5,6,7-hexahydro-s-indacen-4-yl)-4H-1,2,4-triazol-3-amine

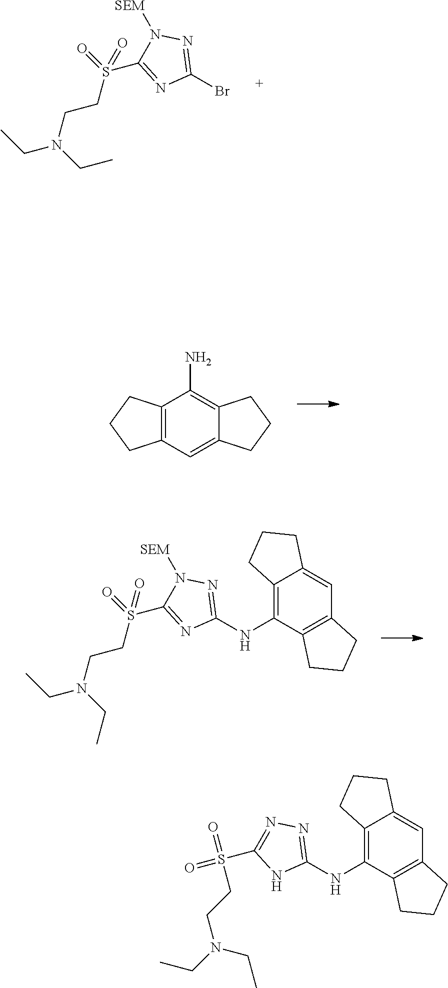 [SMILES: CCN(CC)CCS(=O)(=O)c1nc(Br)nn1COCC[Si](C)(C)C ; Nc1c2c(cc3c1CCC3)CCC2 ; CCN(CC)CCS(=O)(=O)c1nc(Nc2c3c(cc4c2CCC4)CCC3)nn1COCC[Si](C)(C)C ; CCN(CC)CCS(=O)(=O)c1nnc(Nc2c3c(cc4c2CCC4)CCC3)[nH]1]

Step A: Prepared according to the general procedure of N-(1,2,3,5,6,7-hexahydro-s-indacen-4-yl)-5-(phenylsulfonyl)-1H-1,2,4-triazol-3-amine (Example 40, Step A) from 2-((3-bromo-1-((2-(trimethylsilyl)ethoxy)methyl)-1H-1,2,4-triazol-5-yl)sulfonyl)-N,N-diethylethanamine (Intermediate A5) and 1,2,3,5,6,7-hexahydro-s-indacen-4-amine to afford 5-((2-(diethylamino)ethyl)sulfonyl)-N-(1,2,3,5,6,7-hexahydro-s-indacen-4-yl)-1-((2-(trimethylsilyl)ethoxy)methyl)-1H-1,2,4-triazol-3-amine (0.10 g, 50% yield) as a yellow oil.

LCMS m/z 534.3 (M+H)⁺ (ES⁺); 532.0 (M−H)⁻ (ES⁻).

¹H NMR (DMSO-d₆) δ 8.45 (s, 1H), 6.88 (s, 1H), 5.54 (s, 2H), 3.67 (t, J=6.8 Hz, 2H), 3.65-3.59 (m, 2H), 2.87-2.76 (m, 6H), 2.66 (t, J=7.3 Hz, 4H), 2.36 (q, J=7.1 Hz, 4H), 1.94 (p, J=7.5 Hz, 4H), 0.91-0.79 (m, 8H), −0.04 (s, 9H).

Step B: TFA (0.5 mL) was added to a solution of 5-((2-(diethylamino)ethyl)sulfonyl)-N-(1,2,3,5,6,7-hexahydro-s-indacen-4-yl)-1-((2-(trimethylsilyl)ethoxy)methyl)-1H-1,2,4-triazol-3-amine (45 mg, 0.084 mmol) in DCM (2 mL) at RT and the reaction was stirred for 1 h and concentrated in vacuo. The residue was dissolved in Et₂NH (1 mL) and stirred at RT for 17 h. The reaction mixture was concentrated in vacuo and purified by chromatography on silica gel (4 g column, 0-10% then 0-2% (0.7 M ammonia/MeOH)/DCM) to afford the title compound (16 mg, 45% yield) as a pale green solid.

LCMS m/z 404.2 (M+H)⁺ (ES⁺); 402.1 (M−H)⁻ (ES⁻).

¹H NMR (at 363 K, DMSO-d₆) δ 8.07 (s, 1H), 6.89 (s, 1H), 3.37-3.31 (m, 2H), 2.87-2.71 (m, 6H), 2.61 (t, J=7.4 Hz, 4H), 2.39 (q, J=7.1 Hz, 4H), 1.95 (p, J=7.4 Hz, 4H), 0.89 (t, J=7.1 Hz, 6H). One exchangeable proton not observed.

¹H NMR (at 293 K, DMSO-d₆) broad spectrum: δ 7.04-6.74 (m, 1H), 2.88-2.67 (m, 6H), 2.69-2.55 (m, 4H), 2.39 (q, J=7.2 Hz, 4H), 2.01-1.83 (m, 4H), 0.89 (t, J=7.1 Hz, 6H). CH₂ signal obscured by DMSO signal, 2 exchangeable signals not observed.

### Example 47:3-((5-((1,2,3,5,6,7-hexahydro-s-indacen-4-yl)amino)-4H-1,2,4-triazol-3-yl)sulfonyl)propan-1-ol

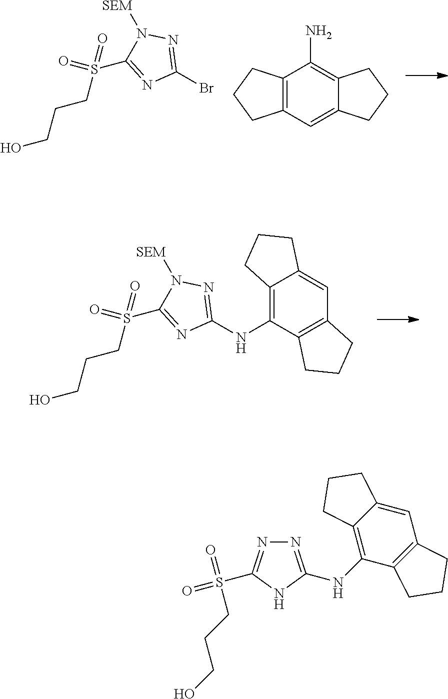

Step A: Prepared according to the general procedure of N-(1,2,3,5,6,7-hexahydro-s-indacen-4-yl)-5-(phenylsulfonyl)-1H-1,2,4-triazol-3-amine (Example 40, Step A) from 3-((3-bromo-1-((2-(trimethylsilyl)ethoxy)methyl)-1H-1,2,4-triazol-5-yl)sulfonyl)propan-1-ol (Intermediate A6) and 1,2,3,5,6,7-hexahydro-s-indacen-4-amine to afford 3-((3-((1,2,3,5,6,7-hexahydro-s-indacen-4-yl)amino)-1-((2-(trimethylsilyl)ethoxy)methyl)-1H-1,2,4-triazol-5-yl)sulfonyl)propan-1-ol (285 mg, 44% yield) as a brown oil.

¹H NMR (DMSO-d₆) δ 8.50 (s, 1H), 6.89 (s, 1H), 5.56 (s, 2H), 3.63-3.56 (m, 4H), 3.50-3.45 (m, 2H), 2.81 (t, J=7.3 Hz, 4H), 2.66 (t, J=7.4 Hz, 4H), 1.95 (p, J=7.5 Hz, 4H), 1.87-1.76 (m, 2H), 0.90-0.83 (m, 2H), −0.03 (s, 9H). One exchangeable proton not observed.

Step B: 3-((5-((1,2,3,5,6,7-hexahydro-s-indacen-4-yl)amino)-1-((2-(trimethylsilyl)-ethoxy)methyl)-1H-1,2,4-triazol-3-yl)sulfonyl)propan-1-ol (30 mg, 0.061 mmol) was dissolved in MeCN (2 mL) and water (0.1 mL). SCX (300 mg) was added and the reaction was stirred at RT for 4 h. The reaction mixture was then diluted with MeOH (5 mL), filtered and washed with MeOH (5 mL). The solvent was evaporated in vacuo and the product was purified by chromatography on silica gel (12 g column, 0-10% (0.7 M ammonia/MeOH)/DCM) to afford the title compound (8 mg, 36% yield) as a colourless solid.

LCMS m/z 363.3 (M+H)⁺ (ES⁺); 361.0 (M−H)⁻ (ES⁻).

¹H NMR (DMSO-d₆) δ 13.08 (br s, 1H), 8.95 (s, 1H), 6.97 (s, 1H), 4.68 (t, J=5.3 Hz, 1H), 3.49-3.43 (m, 2H), 3.36-3.33 (m, assumed 2H, obscured by water peak), 2.83 (t, J=7.4 Hz, 4H), 2.63 (t, J=7.3 Hz, 4H), 1.98 (p, J=7.4 Hz, 4H), 1.82-1.73 (m, 2H).

### Example 48: 5-((3-(Dimethylamino)propyl)sulfonyl)-N-(1,2,3,5,6,7-hexahydro-s-indacen-4-yl)-4H-1,2,4-triazol-3-amine

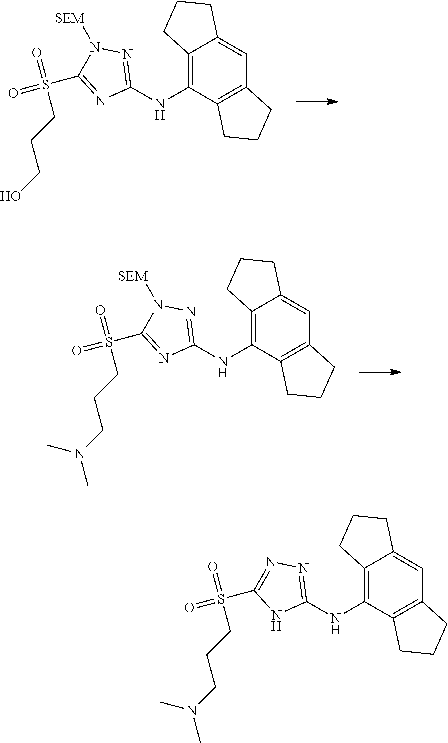 [SMILES: C[Si](C)(C)CCOCn1nc(Nc2c3c(cc4c2CCC4)CCC3)nc1S(=O)(=O)CCCO ; CN(C)CCCS(=O)(=O)c1nc(Nc2c3c(cc4c2CCC4)CCC3)nn1COCC[Si](C)(C)C ; CN(C)CCCS(=O)(=O)c1nnc(Nc2c3c(cc4c2CCC4)CCC3)[nH]1]

3-((3-((1,2,3,5,6,7-hexahydro-s-indacen-4-yl)amino)-1-((2-(trimethylsilyl)ethoxy)-methyl)-1H-1,2,4-triazol-5-yl)sulfonyl)propan-1-ol (Example 47, Step A) (0.25 g, 0.507 mmol) was dissolved in THF (2 mL) and DIPEA (0.133 mL, 0.759 mmol) was added. The reaction was cooled to 0° C., MsCl (0.046 mL, 0.596 mmol) was added dropwise and the reaction stirred for 2 h. KI (9 mg, 0.054 mmol) and dimethylamine (2 M in THF, 2.71 mL, 5.42 mmol) were added and the reaction was heated at 60° C. for 20 h. An additional portion of dimethylamine (2 M in THF, 1.36 mL, 2.71 mmol) was added and the reaction heated at 60° C. for 3 h. After cooling, the reaction was concentrated in vacuo. The resulting residue was passed through a column of SCX (5 g, eluting with 7 N NH₃ in MeOH) and the ammoniacal solution was concentrated in vacuo. The crude product was purified by acidic prep HPLC (10-40% MeCN in water) to afford the title compound (55 mg, 25% yield) as a white solid.

LCMS m/z 390.3 (M+H)⁺ (ES⁺).

¹H NMR (DMSO-d₆) δ 9.00 (s, 1H), 6.97 (s, 1H), 3.40-3.37 (m, 2H), 2.83 (t, J=7.4 Hz, 4H), 2.64-2.62 (m, 6H), 2.34 (s, 6H), 1.97 (p, J=7.4 Hz, 4H), 1.86 (p, J=7.2 Hz, 2H).

One exchangeable proton not observed.

### Example 49: N-(1,2,3,5,6,7-hexahydro-s-indacen-4-yl)-5-((3-(pyrrolidin-1-yl)propyl)sulfonyl)-4H-1,2,4-triazol-3-amine

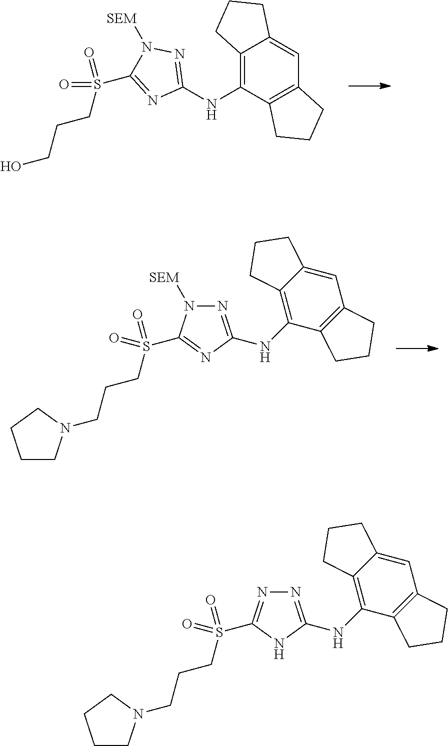 [SMILES: C[Si](C)(C)CCOCn1nc(Nc2c3c(cc4c2CCC4)CCC3)nc1S(=O)(=O)CCCO ; C[Si](C)(C)CCOCn1nc(Nc2c3c(cc4c2CCC4)CCC3)nc1S(=O)(=O)CCCN1CCCC1 ; O=S(=O)(CCCN1CCCC1)c1nnc(Nc2c3c(cc4c2CCC4)CCC3)[nH]1]

Prepared according to the general procedure of 5-((3-(dimethylamino)propyl)sulfonyl)-N-(1,2,3,5,6,7-hexahydro-s-indacen-4-yl)-4H-1,2,4-triazol-3-amine (Example 48) from 3-((3-((1,2,3,5,6,7-hexahydro-s-indacen-4-yl)amino)-1-((2-(trimethylsilyl)-ethoxy)methyl)-1H-1,2,4-triazol-5-yl)sulfonyl)propan-1-ol (Example 47, Step A) and pyrrolidine to afford the title compound (32 mg, 14% yield) as a white solid.

LCMS m/z 416.1 (M+H)⁺ (ES⁺).

¹H NMR (DMSO-d₆) δ 8.93 (s, 1H), 6.96 (s, 1H), 3.36-3.32 (m, assumed 2H, obscured by water peak), 2.83 (t, J=7.4 Hz, 4H), 2.63 (t, J=7.3 Hz, 4H), 2.44 (t, J=6.9 Hz, 2H), 2.37-2.35 (m, 4H), 1.98 (p, J=7.4 Hz, 4H), 1.81-1.75 (m, 2H), 1.67-1.64 (m, 4H). One exhangeable proton not observed.

### Example 50: N-(1,2,3,5,6,7-hexahydro-s-indacen-4-yl)-5-((3-morpholinopropyl)sulfonyl)-4H-1,2,4-triazol-3-amine

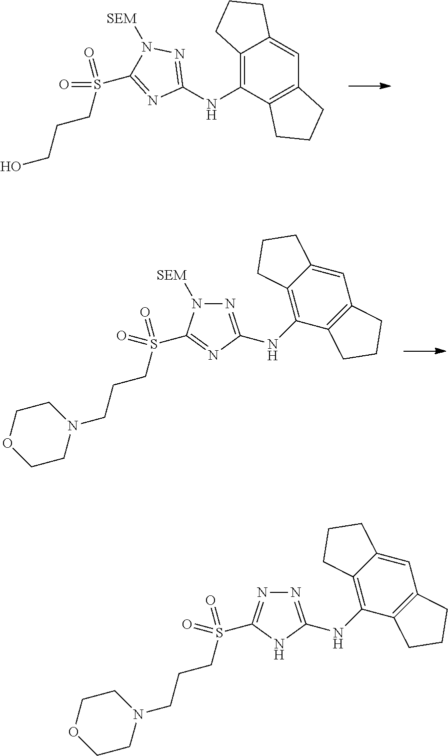 [SMILES: C[Si](C)(C)CCOCn1nc(Nc2c3c(cc4c2CCC4)CCC3)nc1S(=O)(=O)CCCO ; C[Si](C)(C)CCOCn1nc(Nc2c3c(cc4c2CCC4)CCC3)nc1S(=O)(=O)CCCN1CCOCC1 ; O=S(=O)(CCCN1CCOCC1)c1nnc(Nc2c3c(cc4c2CCC4)CCC3)[nH]1]

Prepared according to the general procedure of 5-((3-(dimethylamino)propyl)sulfonyl)-N-(1,2,3,5,6,7-hexahydro-s-indacen-4-yl)-4H-1,2,4-triazol-3-amine (Example 48) from 3-((3-((1,2,3,5,6,7-hexahydro-s-indacen-4-yl)amino)-1-((2-(trimethylsilyl)-ethoxy)methyl)-1H-1,2,4-triazol-5-yl)sulfonyl)propan-1-ol (Example 47, Step A) and morpholine to afford the title compound (45 mg, 19% yield) as a white solid.

LCMS m/z 432.1 (M+H)⁺ (ES⁺).

¹H NMR (DMSO-d₆) δ 12.66 (br s, 1H), 8.93 (s, 1H), 6.96 (s, 1H), 3.53 (t, J=4.6 Hz, 4H), 3.36-3.32 (m, assumed 2H, obscured by water peak), 2.82 (t, J=7.4 Hz, 4H), 2.62 (t, J=7.6 Hz, 4H), 2.33 (t, J=6.9 Hz, 2H), 2.27 (app br s, 4H), 1.97 (p, J=7.4 Hz, 4H), 1.81-1.75 (m, 2H).

### Example 51: N-(1,2,3,5,6,7-hexahydro-s-indacen-4-yl)-5-((3-(4-methylpiperazin-1-yl)propyl)sulfonyl)-4H-1,2,4-triazol-3-amine

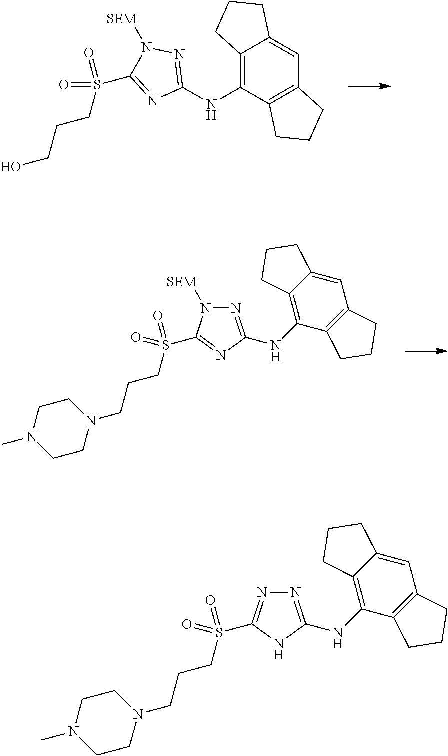 [SMILES: C[Si](C)(C)CCOCn1nc(Nc2c3c(cc4c2CCC4)CCC3)nc1S(=O)(=O)CCCO ; CN1CCN(CCCS(=O)(=O)c2nc(Nc3c4c(cc5c3CCC5)CCC4)nn2COCC[Si](C)(C)C)CC1 ; CN1CCN(CCCS(=O)(=O)c2nnc(Nc3c4c(cc5c3CCC5)CCC4)[nH]2)CC1]

Prepared according to the general procedure of 5-((3-(dimethylamino)propyl)sulfonyl)-N-(1,2,3,5,6,7-hexahydro-s-indacen-4-yl)-4H-1,2,4-triazol-3-amine (Example 48) from 3-((3-((1,2,3,5,6,7-hexahydro-s-indacen-4-yl)amino)-1-((2-(trimethylsilyl)-ethoxy)methyl)-1H-1,2,4-triazol-5-yl)sulfonyl)propan-1-ol (Example 47, Step A) and 1-methylpiperazine to afford the title compound (33 mg, 14% yield) as a white solid.

LCMS m/z 445.1 (M+H)⁺ (ES⁺).

¹H NMR (DMSO-d₆) δ 13.06 (br s, 1H), 8.96 (s, 1H), 6.96 (s, 1H), 3.36-3.29 (m, assumed 2H, obscured by water peak), 2.82 (t, J=7.4 Hz, 4H), 2.62 (t, J=7.2 Hz, 4H), 2.33-2.28 (m, 10H), 2.14 (s, 3H), 1.97 (p, J=7.4 Hz, 4H), 1.76 (p, J=6.9 Hz, 2H).

### Example 52: N¹-(3-((5-((1,2,3,5,6,7-hexahydro-s-indacen-4-yl)amino)-4H-1,2,4-triazol-3-yl)sulfonyl)propyl)-N¹,N²,N²-trimethylethane-1,2-diamine

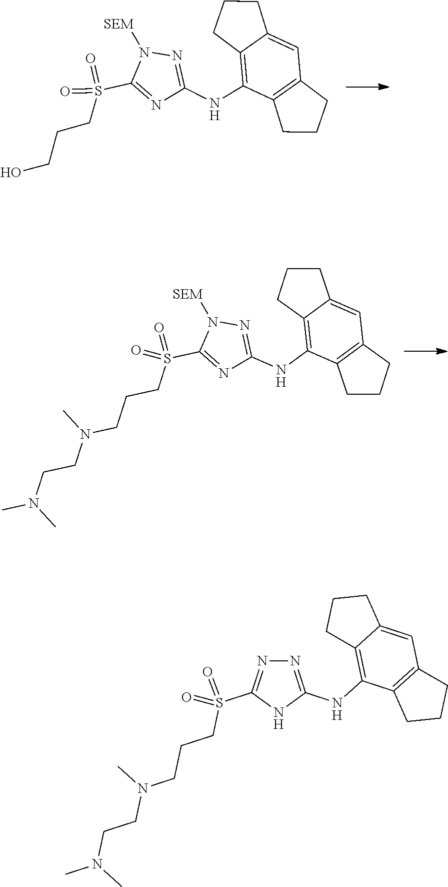 [SMILES: C[Si](C)(C)CCOCn1nc(Nc2c3c(cc4c2CCC4)CCC3)nc1S(=O)(=O)CCCO ; CN(C)CCN(C)CCCS(=O)(=O)c1nc(Nc2c3c(cc4c2CCC4)CCC3)nn1COCC[Si](C)(C)C ; CN(C)CCN(C)CCCS(=O)(=O)c1nnc(Nc2c3c(cc4c2CCC4)CCC3)[nH]1]

Prepared according to the general procedure of 5-((3-(dimethylamino)propyl)sulfonyl)-N-(1,2,3,5,6,7-hexahydro-s-indacen-4-yl)-4H-1,2,4-triazol-3-amine (Example 48) from 3-((3-((1,2,3,5,6,7-hexahydro-s-indacen-4-yl)amino)-1-((2-(trimethylsilyl)-ethoxy)methyl)-1H-1,2,4-triazol-5-yl)sulfonyl)propan-1-ol (Example 47, Step A) and N¹,N¹,N²-trimethylethane-1,2-diamine to afford the title compound (30 mg, 13% yield) as a white solid.

LCMS m/z 447.4 (M+H)⁺ (ES⁺).

¹H NMR (DMSO-d₆): δ 8.84 (s, 1H), 6.94 (s, 1H), 3.32-3.29 (m, 2H), 2.82 (t, J=7.4 Hz, 4H), 2.62 (t, J=7.3 Hz, 4H), 2.38-2.29 (m, 6H), 2.13 (s, 6H), 2.09 (s, 3H), 1.96 (p, J=7.4 Hz, 4H), 1.76-1.71 (m, 2H). One exchangeable proton not observed.

### Example 53: N-(1,2,3,5,6,7-hexahydro-s-indacen-4-yl)-5-((3-(methyl((1-methylpiperidin-4-yl)methyl)amino)propyl)sulfonyl)-4H-1,2,4-triazol-3-amine

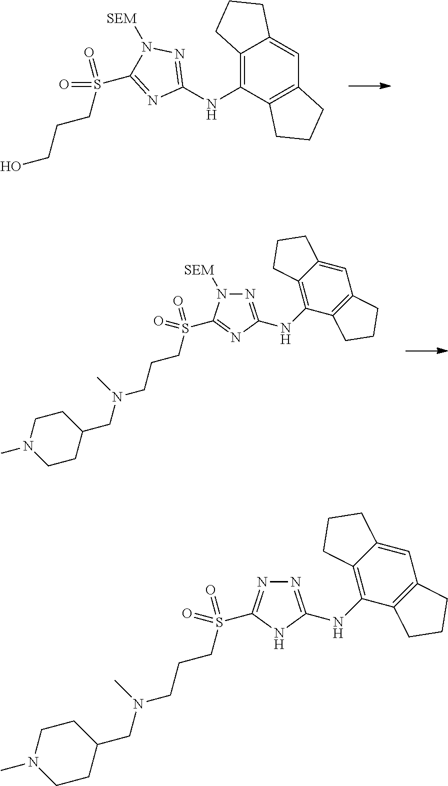 [SMILES: C[Si](C)(C)CCOCn1nc(Nc2c3c(cc4c2CCC4)CCC3)nc1S(=O)(=O)CCCO ; CN1CCC(CN(C)CCCS(=O)(=O)c2nc(Nc3c4c(cc5c3CCC5)CCC4)nn2COCC[Si](C)(C)C)CC1 ; CN1CCC(CN(C)CCCS(=O)(=O)c2nnc(Nc3c4c(cc5c3CCC5)CCC4)[nH]2)CC1]

Prepared according to the general procedure of 5-((3-(dimethylamino)propyl)sulfonyl)-N-(1,2,3,5,6,7-hexahydro-s-indacen-4-yl)-4H-1,2,4-triazol-3-amine (Example 48) from 3-((3-((1,2,3,5,6,7-hexahydro-s-indacen-4-yl)amino)-1-((2-(trimethylsilyl)-ethoxy)methyl)-1H-1,2,4-triazol-5-yl)sulfonyl)propan-1-ol (Example 47, Step A) and N-methyl-1-(1-methylpiperidin-4-yl)methanamine to afford the title compound (25 mg, 10% yield) as a white solid.

LCMS m/z 487.5 (M+H)⁺ (ES⁺).

¹H NMR (DMSO-d₆) δ 8.87 (s, 1H), 6.93 (s, 1H), 3.31-3.28 (m, 2H), 2.82 (t, J=7.4 Hz, 4H), 2.73-2.71 (m, 2H), 2.62 (t, J=7.4 Hz, 4H), 2.32 (t, J=6.8 Hz, 2H), 2.14 (s, 3H), 2.05 (s, 3H), 1.97 (p, J=7.4 Hz, 4H), 1.85-1.80 (m, 2H), 1.76-1.71 (m, 2H), 1.61-1.59 (m, 2H), 1.37-1.33 (m, 1H), 1.08-1.00 (m, 2H). One CH₂ obscured by DMSO peak, one exchangeable proton not observed.

### Example 54: N-(1,2,3,5,6,7-hexahydro-s-indacen-4-yl)-5-((3-(methylamino)propyl)sulfonyl)-4H-1,2,4-triazol-3-amine

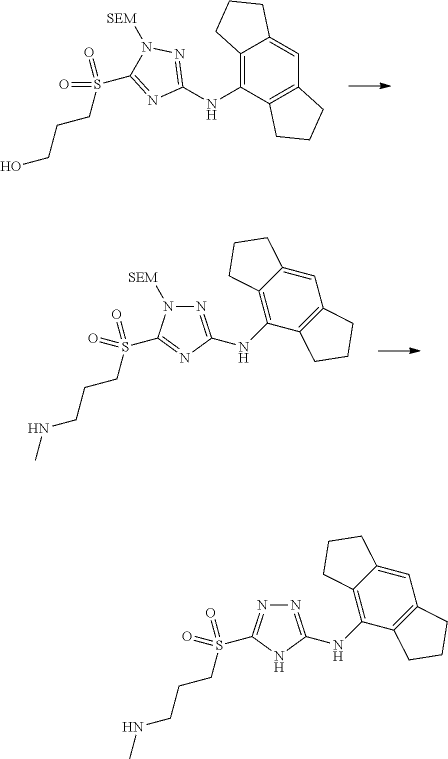 [SMILES: C[Si](C)(C)CCOCn1nc(Nc2c3c(cc4c2CCC4)CCC3)nc1S(=O)(=O)CCCO ; CNCCCS(=O)(=O)c1nc(Nc2c3c(cc4c2CCC4)CCC3)nn1COCC[Si](C)(C)C ; CNCCCS(=O)(=O)c1nnc(Nc2c3c(cc4c2CCC4)CCC3)[nH]1]

Prepared according to the general procedure of 5-((3-(dimethylamino)propyl)sulfonyl)-N-(1,2,3,5,6,7-hexahydro-s-indacen-4-yl)-4H-1,2,4-triazol-3-amine (Example 48) from 3-((3-((1,2,3,5,6,7-hexahydro-s-indacen-4-yl)amino)-1-((2-(trimethylsilyl)-ethoxy)methyl)-1H-1,2,4-triazol-5-yl)sulfonyl)propan-1-ol (Example 47, Step A) and methylamine (2 M in THF) to afford the title compound (49 mg, 26% yield) as a white solid.

LCMS m/z 376.1 (M+H)⁺ (ES⁺).

¹H NMR (DMSO-d₆) δ 8.61 (s, 1H), 6.90 (s, 1H), 3.33-3.30 (m, 2H), 2.80 (t, J=7.4 Hz, 4H), 2.63-2.58 (m, 6H), 2.26 (s, 3H), 1.95 (p, J=7.4 Hz, 4H), 1.78 (p, J=7.0 Hz, 2H). Two exchangeable protons not observed.

### Example 55: 5-((3-(ethylamino)propyl)sulfonyl)-N-(1,2,3,5,6,7-hexahydro-s-indacen-4-yl)-4H-1,2,4-triazol-3-amine

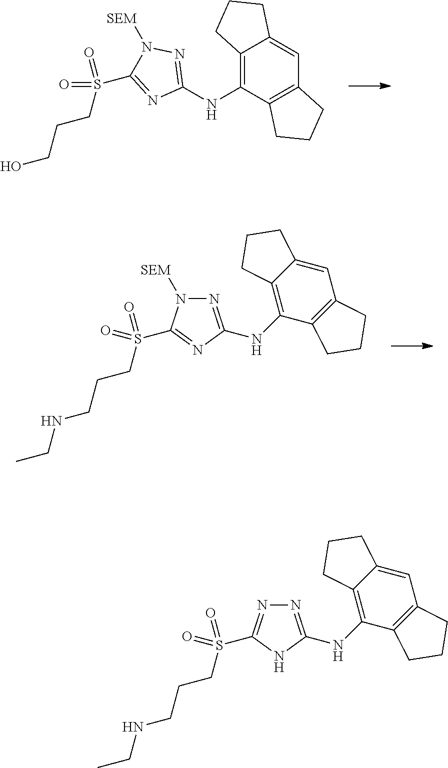 [SMILES: C[Si](C)(C)CCOCn1nc(Nc2c3c(cc4c2CCC4)CCC3)nc1S(=O)(=O)CCCO ; CCNCCCS(=O)(=O)c1nc(Nc2c3c(cc4c2CCC4)CCC3)nn1COCC[Si](C)(C)C ; CCNCCCS(=O)(=O)c1nnc(Nc2c3c(cc4c2CCC4)CCC3)[nH]1]

Prepared according to the general procedure of 5-((3-(dimethylamino)propyl)sulfonyl)-N-(1,2,3,5,6,7-hexahydro-s-indacen-4-yl)-4H-1,2,4-triazol-3-amine (Example 48) from 3-((3-((1,2,3,5,6,7-hexahydro-s-indacen-4-yl)amino)-1-((2-(trimethylsilyl)-ethoxy)methyl)-1H-1,2,4-triazol-5-yl)sulfonyl)propan-1-ol (Example 47, Step A) and ethylamine (2 M in THF) to afford the title compound (46 mg, 26% yield) as a white solid.

LCMS m/z 390.1 (M+H)⁺ (ES⁺).

¹H NMR (DMSO-d₆) δ 8.73 (s, 1H), 6.91 (s, 1H), 3.45-3.37 (m, 2H), 2.85-2.76 (m, 6H), 2.71 (q, J=7.2 Hz, 2H), 2.63 (t, J=7.2 Hz, 4H), 2.35 (s, 1H), 1.96-1.86 (m, 6H), 1.10 (t, J=7.2 Hz, 3H). One exchangeable proton not observed.

### Example 56: 5-((3-(cyclopropylamino)propyl)sulfonyl)-N-(1,2,3,5,6,7-hexahydro-s-indacen-4-yl)-4H-1,2,4-triazol-3-amine

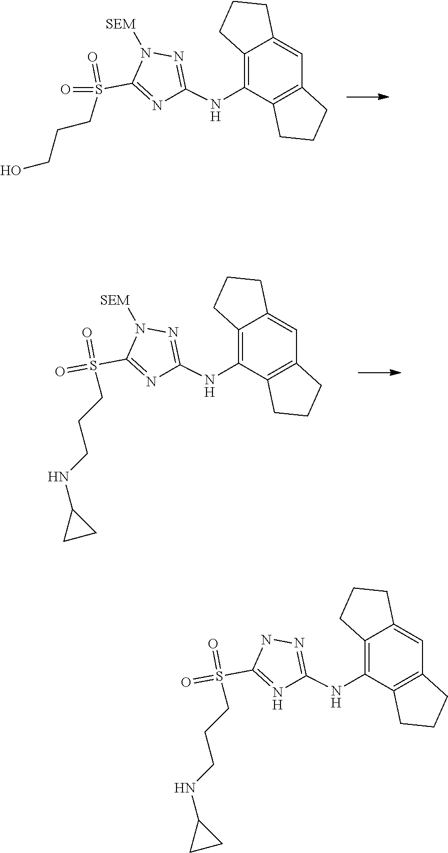

Prepared according to the general procedure of 5-((3-(dimethylamino)propyl)sulfonyl)-N-(1,2,3,5,6,7-hexahydro-s-indacen-4-yl)-4H-1,2,4-triazol-3-amine (Example 48) from 3-((3-((1,2,3,5,6,7-hexahydro-s-indacen-4-yl)amino)-1-((2-(trimethylsilyl)-ethoxy)methyl)-1H-1,2,4-triazol-5-yl)sulfonyl)propan-1-ol (Example 47, Step A) and cyclopropanamine to afford the title compound (7 mg, 4% yield) as a white solid.

LCMS m/z 402.1 (M+H)⁺ (ES⁺).

¹H NMR (DMSO-d₆) δ 8.99 (s, 1H), 8.14 (s, 1H), 6.97 (s, 1H), 3.36-3.33 (m, assumed 2H, obscured by water peak), 2.83 (t, J=7.4 Hz, 4H), 2.70-2.69 (m, 2H), 2.63 (t, J=7.3 Hz, 4H), 2.08 (app. br. s, 1H), 1.97 (p, J=7.4 Hz, 4H), 1.80-1.77 (m, 2H), 0.41-0.35 (m, 2H), 0.28-0.21 (m, 2H). One exchangeable proton not observed.

### Example 57: N-(1,2,3,5,6,7-hexahydro-s-indacen-4-yl)-3-((morpholin-2-ylmethyl)sulfonyl)-1H-1,2,4-triazol-5-amine

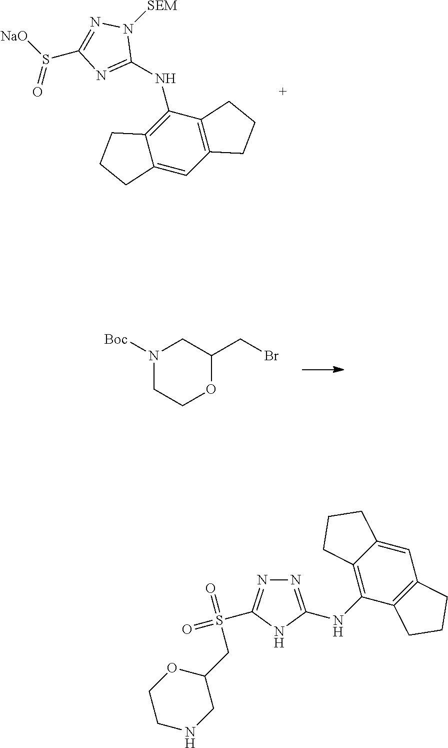

Sodium 5-((1,2,3,5,6,7-hexahydro-s-indacen-4-yl)amino)-1-((2-(trimethylsilyl)ethoxy)-methyl)-1H-1,2,4-triazole-3-sulfinate (Intermediate B2) (200 mg, 0.438 mmol) was dissolved in DMF (2 mL) and tert-butyl 2-(bromomethyl)morpholine-4-carboxylate (123 mg, 0.438 mmol), Cs₂CO₃ (143 mg, 0.438 mmol) and KI (7.3 mg, 0.044 mmol) were added. The reaction was warmed up to 50° C. for 16 h and then to 100° C. and stirred for 4 h. The reaction was diluted with 2 M NaOH (aq) (20 mL) and extracted with DCM (2×20 mL). The combined organic extracts were dried (phase separator) and concentrated in vacuo. The crude product was dissolved in HCl (4 M in 1,4-dioxane, 2 mL) and stirred for 16 h at RT. The volatiles were evaporated and the product purified by acidic prep HPLC (35-65% MeOH in water) to afford the title compound (3 mg, 2% yield) as a white solid.

LCMS m/z 404.4 (M+H)⁺ (ES⁺).

¹H NMR (DMSO-d₆) δ 9.00 (s, 1H), 6.97 (s, 1H), 4.05-3.89 (m, 1H), 3.74-3.68 (m, 1H), 3.59 (dd, J=14.8, 4.5 Hz, 1H), 3.54-3.43 (m, 3H), 3.04-2.98 (m, 1H), 2.83 (m, 5H), 2.76-2.69 (m, 1H), 2.64 (m, 5H), 1.98 (p, J=7.4 Hz, 4H). One exchangeable proton not observed.

### Example 58: N-(1,2,3,5,6,7-hexahydro-s-indacen-4-yl)-3-(((1-methylpiperidin-3-yl)methyl)sulfonyl)-1H-1,2,4-triazol-5-amine

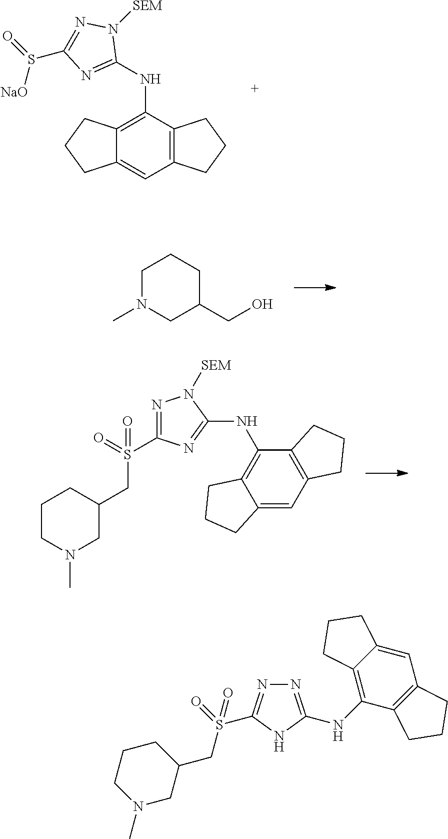

(1-Methylpiperidin-3-yl)methanol (60 mg, 0.464 mmol) was dissolved in THF (2 mL) and cooled to 0° C. DIPEA (0.116 mL, 0.665 mmol) was added, followed by MsCl (0.038 mL, 0.488 mmol) and the reaction was slowly warmed to RT, then stirred for 2 h. The reaction was quenched with sat aq NaHCO₃ (20 mL) and extracted with EtOAc (2×15 mL). The combined organics were washed with brine (30 mL), dried (phase separator) and concentrated in vacuo. The resulting residue was dissolved in DMSO (1 mL) and added to a suspension of KI (10 mg, 0.060 mmol) and sodium 5-((1,2,3,5,6,7-hexahydro-s-indacen-4-yl)amino)-1-((2-(trimethylsilyl)ethoxy)methyl)-1H-1,2,4-triazole-3-sulfinate (Intermediate B2) (0.2 g, 0.438 mmol) in DMSO (1 mL). The reaction was stirred at 60° C. for 16 h, then at 80° C. for 4 h. The reaction was quenched with aq 2 M NaOH (3 mL) and water (6 mL), then extracted with EtOAc (2×30 mL). The combined organics were washed with brine (30 mL), dried (phase separator) and concentrated in vacuo. The residue was dissolved in HCl (4 M in dioxane, 0.016 mL, 0.443 mmol) and stirred at RT for 16 h. The reaction was concentrated in vacuo and the product purified by acidic prep HPLC (20-50% MeOH in water) to afford the title compound (5 mg, 3% yield) as a white powder.

LCMS m/z 416.3 (M+H)⁺ (ES⁺); 414.3 (M−H)⁻ (ES⁻).

¹H NMR (DMSO-d₆) δ 13-15 (s, 1H), 9.00 (s, 1H), 6.97 (s, 1H), 2.90-2.73 (m, 5H), 2.63 (t, J=7.4 Hz, 4H), 2.26-2.18 (m, 2H), 2.16-2.04 (m, 2H) 1.97 (p, J=7.4 Hz, 4H), 1.73-1.65 (m, 1H), 1.64-1.57 (m, 1H) 1.49-1.38 (m, 1H), 1.17-1.05 (m, 1H). One CH₃ and one CH₂ obscured by solvent.

### Example 59: N-(1,2,3,5,6,7-Hexahydro-s-indacen-4-yl)-3-((2-(1-methylpiperidin-2-yl)ethyl)sulfonyl)-1H-1,2,4-triazol-5-amine

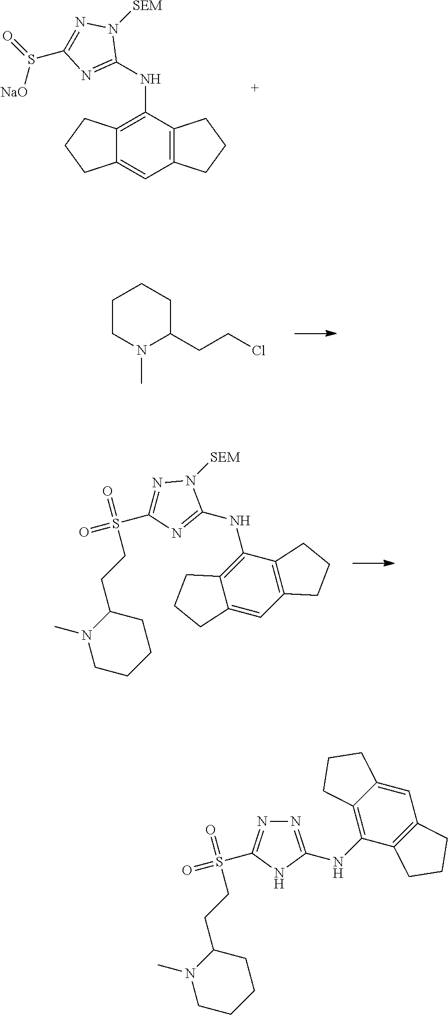

Sodium 5-((1,2,3,5,6,7-hexahydro-s-indacen-4-yl)amino)-1-((2-(trimethylsilyl)ethoxy)-methyl)-1H-1,2,4-triazole-3-sulfinate (Intermediate B2) (200 mg, 0.438 mmol), Cs₂CO₃ (150 mg, 0.460 mmol) and KI (10 mg, 0.060 mmol) were dissolved in DMSO (2 mL) and 2-(2-chloroethyl)-1-methylpiperidine (80 mg, 0.495 mmol) was added. The reaction was heated to 50° C. for 16 h and then diluted with aq 1 M NaOH (30 mL) and extracted with EtOAc (2×30 mL). The combined organics were washed with brine (50 mL), dried (phase separator) and concentrated in vacuo. The residue was dissolved in HCl (4 M in dioxane, 4 mL, 16 mmol) and stirred at RT for 16 h. The reaction was concentrated in vacuo and the product was purified by acidic prep HPLC (20-50% MeOH in water) to afford the title compound (4 mg, 2% yield) as a white powder.

LCMS m/z 430.3 (M+H)⁺ (ES⁺); 428.4 (M−H)⁻ (ES⁻).

¹H NMR (DMSO-d₆) δ 13-19 (s, 1H), 9.02 (s, 1H), 6.98 (s, 1H), 2.84 (t, J=7.3 Hz, 4H), 2.65 (t, J=7.3 Hz, 4H), 2.37 (p, J=1.9 Hz, 1H), 1.98 (p, J=7.3 Hz, 4H), 1.94-1.84 (m, 2H), 1.69-1.20 (m, 8H). One CH₃ and one CH₂ obscured by solvent.

### Example 60: N-(1,2,3,5,6,7-hexahydro-s-indacen-4-yl)-3-((2-(1-methylpyrrolidin-2-yl)ethyl)sulfonyl)-1H-1,2,4-triazol-5-amine

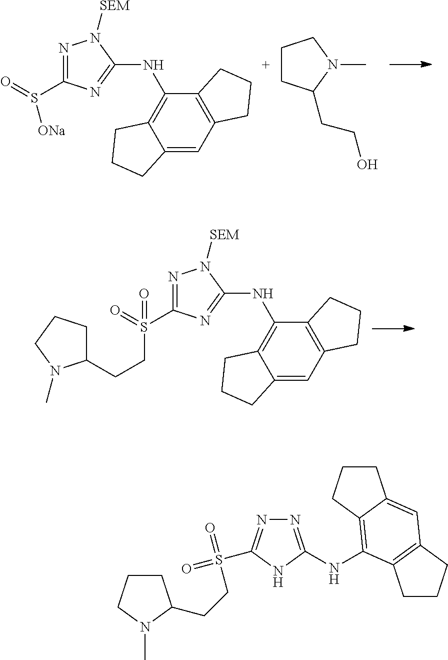

2-(1-Methylpyrrolidin-2-yl)ethanol (0.340 g, 2.63 mmol) was dissolved in THF (2 mL). To this was added N-ethyl-N-isopropylpropan-2-amine (0.732 mL, 4.20 mmol) followed by MsCl (0.244 mL, 3.15 mmol) at 0° C. The reaction was warmed to RT, stirred for 4 h then diluted with sat aq NaHCO₃ (25 mL) and extracted with DCM (2×15 mL). The combined organics were dried (phase separator) and concentrated in vacuo. The residue was dissolved in DMSO (1 mL) and added to a suspension of sodium 5-((1,2,3,5,6,7-hexahydro-s-indacen-4-yl)amino)-1-((2-(trimethylsilyl)ethoxy)-methyl)-1H-1,2,4-triazole-3-sulfinate (Intermediate B2) (400 mg, 0.876 mmol) and KI (20 mg, 0.120 mmol) in DMSO (1 mL). The reaction was stirred for 16 h at 50° C., then diluted with aq 1 M NaOH (20 mL) and extracted with EtOAc (2×25 mL). The combined organics were dried (phase separator) and concentrated in vacuo. The product was purified by chromatography on silica gel (12 g column, 0-5% MeOH/DCM) to afford N-(1,2,3,5,6,7-hexahydro-s-indacen-4-yl)-3-((2-(1-methylpyrrolidin-2-yl)ethyl)sulfonyl)-1-((2-(trimethylsilyl)ethoxy)methyl)-1H-1,2,4-triazol-5-amine as a colourless oil. It was then dissolved in HCl (4 M in dioxane, 4 mL, 16 mmol) and stirred at RT for 16 h. The reaction was concentrated in vacuo and the product was purified by acid preparative HPLC (20-50% MeOH in water) to afford the title compound (10 mg, 3% yield) as a white powder.

LCMS m/z 416.3 (M+H)⁺ (ES⁺); 414.3 (M−H)⁻ (ES⁻).

¹H NMR (DMSO-d₆) δ 9.10 (s, 1H), 6.97 (s, 1H), 3.32 (t, J=8.0 Hz, 2H), 3.00-2.94 (m, 1H), 2.83 (t, J=7.4 Hz, 4H), 2.64 (t, J=7.3 Hz, 4H), 2.30-2.23 (m, 1H), 2.21-2.12 (m, 4H), 1.98 (p, J=7.3 Hz, 4H), 1.92-1.80 (m, 2H), 1.68-1.55 (m, 3H), 1.42-1.33 (m, 1H). One exchangeable proton not observed.

### Example 61: N-(1,2,3,5,6,7-hexahydro-s-indacen-4-yl)-3-(((1-methylpyrrolidin-2-yl)methyl)sulfonyl)-1H-1,2,4-triazol-5-amine

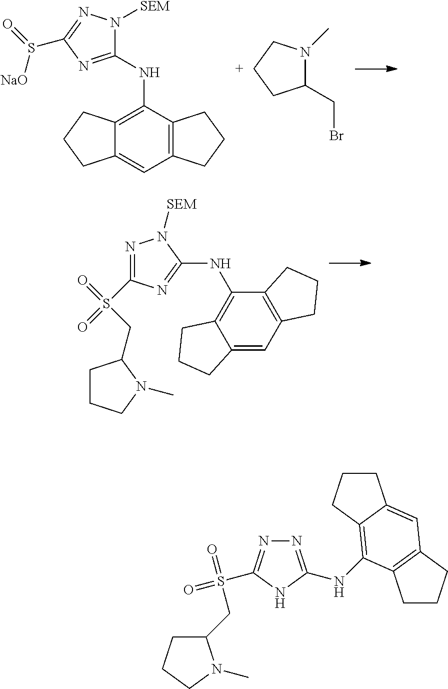

Prepared according to the general procedure of N-(1,2,3,5,6,7-hexahydro-s-indacen-4-yl)-3-((2-(1-methylpiperidin-2-yl)ethyl)sulfonyl)-1H-1,2,4-triazol-5-amine (Example 59) from sodium 5-((1,2,3,5,6,7-hexahydro-s-indacen-4-yl)amino)-1-((2-(trimethylsilyl)ethoxy)methyl)-1H-1,2,4-triazole-3-sulfinate (Intermediate B2) and 2-(bromomethyl)-1-methylpyrrolidine hydrobromide to afford the title compound (3 mg, 2% yield) as a white solid.

LCMS m/z 402.3 (M+H)⁺ (ES⁺); 400.2 (M−H)⁻ (ES⁻).

¹H NMR (DMSO-d₆) δ 9.05 (s, 1H), 6.96 (s, 1H), 2.91-2.86 (m, 1H), 2.83 (t, J=7.6 Hz, 4H), 2.64 (t, J=7.5 Hz, 4H), 2.18 (s, 3H), 2.1-2.04 (m, 1H), 2.02-1.92 (m, 5H), 1.71-1.47 (m, 4H). One exchangeable proton not observed. One CH2 obscured by water peak.

### Example 62: N-(1,2,3,5,6,7-hexahydro-s-indacen-4-yl)-3-((pyrrolidin-2-ylmethyl)sulfonyl)-1H-1,2,4-triazol-5-amine

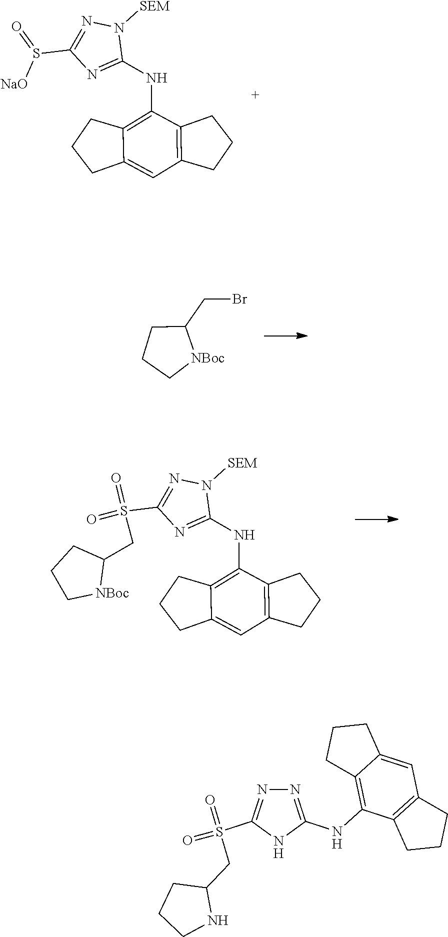

Prepared according to the general procedure of N-(1,2,3,5,6,7-hexahydro-s-indacen-4-yl)-3-((2-(1-methylpiperidin-2-yl)ethyl)sulfonyl)-1H-1,2,4-triazol-5-amine (Example 59) from sodium 5-((1,2,3,5,6,7-hexahydro-s-indacen-4-yl)amino)-1-((2-(trimethylsilyl)ethoxy)methyl)-1H-1,2,4-triazole-3-sulfinate (Intermediate B2) and tert-butyl 2-(bromomethyl)pyrrolidine-1-carboxylate to afford the title compound (22 mg, 12% yield) as a white powder.

LCMS m/z 388.3 (M+H)⁺ (ES⁺); 386.3 (M−H)⁻ (ES⁻).

¹H NMR (DMSO-d₆) δ 9.00 (s, 1H), 6.98 (s, 1H), 3.79-3.66 (m, 3H), 3.07-2.95 (m, 2H), 2.84 (t, J=7.4 Hz, 4H), 2.65 (t, J=7.2 Hz, 4H), 2.07-1.91 (m, 5H), 1.87-1.80 (m, 1H), 1.78-1.70 (m, 1H), 1.61-1.53 (m, 1H). Two exchangeable protons not observed.

### Example 63: N-(1,2,3,5,6,7-hexahydro-s-indacen-4-yl)-3-((2-(pyridin-2-yl)ethyl)sulfonyl)-1H-1,2,4-triazol-5-amine

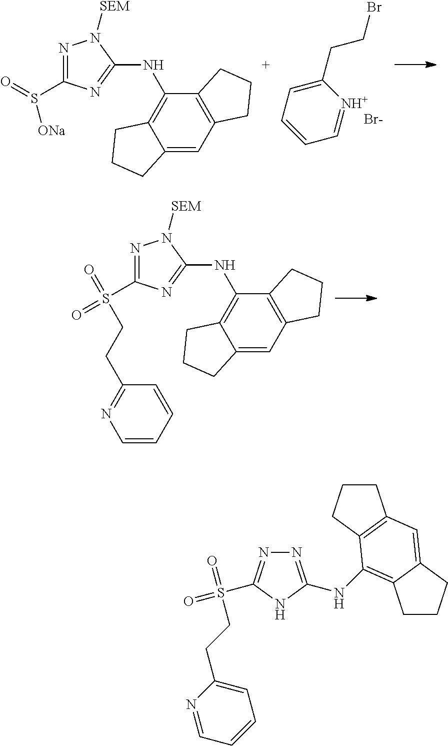

Prepared according to the general procedure of N-(1,2,3,5,6,7-hexahydro-s-indacen-4-yl)-3-((2-(1-methylpiperidin-2-yl)ethyl)sulfonyl)-1H-1,2,4-triazol-5-amine (Example 59) from sodium 5-((1,2,3,5,6,7-hexahydro-s-indacen-4-yl)amino)-1-((2-(trimethylsilyl)ethoxy)methyl)-1H-1,2,4-triazole-3-sulfinate (Intermediate B2) and 2-(2-bromoethyl)pyridin-1-ium bromide to afford the title compound (21 mg, 11% yield) as a white powder.

LCMS m/z 410.3 (M+H)⁺ (ES⁺); 408.2 (M−H)⁻ (ES⁻).

¹H NMR (DMSO-d₆) δ 13.16 (s, 1H), 9.01 (s, 1H), 8.47 (m, 1H), 7.70 (td, J=7.6, 1.9 Hz, 1H), 7.32 (d, J=7.8 Hz, 1H), 7.25-7.21 (m, 1H), 6.96 (s, 1H), 3.81-3.75 (m, 2H), 3.18-3.10 (m, 2H), 2.84 (t, J=7.5 Hz, 4H), 2.65 (t, J=7.4 Hz, 4H), 1.98 (p, J=7.3 Hz, 4H).

### Example 64:3-(Cyclopentylsulfonyl)-N-(1,2,3,5,6,7-hexahydro-s-indacen-4-yl)-1H-1,2,4-triazol-5-amine

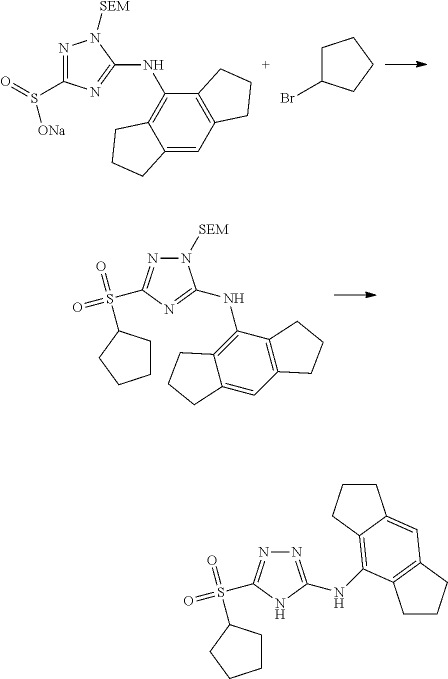

Prepared according to the general procedure of N-(1,2,3,5,6,7-hexahydro-s-indacen-4-yl)-3-((2-(1-methylpiperidin-2-yl)ethyl)sulfonyl)-1H-1,2,4-triazol-5-amine (Example 59) from sodium 5-((1,2,3,5,6,7-hexahydro-s-indacen-4-yl)amino)-1-((2-(trimethylsilyl)ethoxy)methyl)-1H-1,2,4-triazole-3-sulfinate (Intermediate B2) and bromocyclopentane to afford the title compound (13 mg, 8% yield) as a white powder.

LCMS m/z 373.3 (M+H)⁺ (ES⁺); 371.2 (M−H)⁻ (ES⁻).

¹H NMR (DMSO-d₆) δ 8.99 (s, 1H), 6.95 (s, 1H), 3.82-3.73 (m, 1H), 2.82 (t, J=7.3 Hz, 4H), 2.62 (t, J=7.4 Hz, 4H), 2.02-1.82 (m, 8H), 1.64-1.50 (m, 4H). One exchangeable proton not observed.

### Example 65: N-(1,2,3,5,6,7-hexahydro-s-indacen-4-yl)-3-((3-methoxypropyl)sulfonyl)-1H-1,2,4-triazol-5-amine

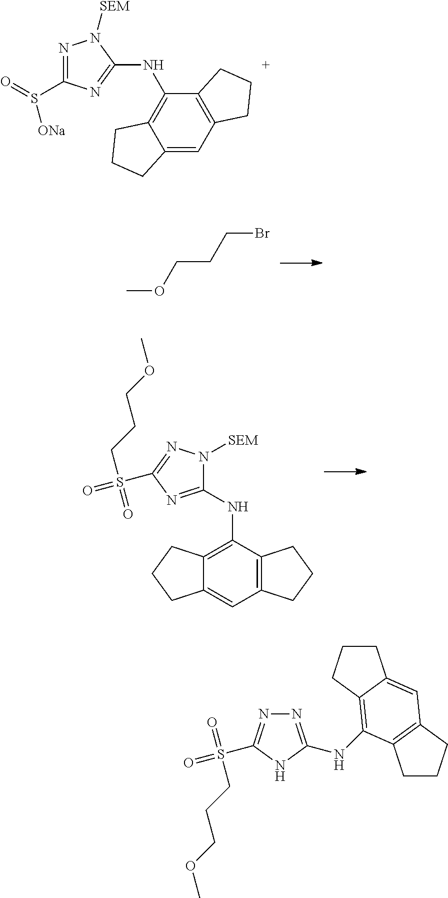

Step A: 5-((1,2,3,5,6,7-hexahydro-s-indacen-4-yl)amino)-1-((2-(trimethylsilyl)ethoxy)-methyl)-1H-1,2,4-triazole-3-sulfinic acid, sodium (Intermediate B2) (200 mg, 0.438 mmol) was dissolved in DMSO (2 mL) and 1-bromo-3-methoxypropane (80 mg, 0.526 mmol) and KI (7 mg, 0.042 mmol) were added. The reaction was warmed up to 50° C. for 16 h. The reaction was diluted with 2 M NaOH (20 mL) and extracted with DCM (2×20 mL). The combined organic extracts were dried (phase separator) and concentrated in vacuo. The crude was purified by chromatography on silica gel (12 g column, 0-100% EtOAc/isohexane) to afford N-(1,2,3,5,6,7-hexahydro-s-indacen-4-yl)-3-((3-methoxypropyl)sulfonyl)-1-((2-(trimethylsilyl)ethoxy)methyl)-1H-1,2,4-triazol-5-amine (79 mg, 28% yield).

LCMS m/z 507.4 (M+H)⁺ (ES⁺).

¹H NMR (DMSO-d₆) δ 8.91 (s, 1H), 6.99 (d, J=5.2 Hz, 1H), 3.66-3.60 (m, 2H), 3.39-3.28 (m, 6H), 3.19 (s, 3H), 2.84 (t, J=7.4 Hz, 4H), 2.65 (t, J=7.3 Hz, 4H), 2.02-1.93 (m, 4H), 1.86-1.75 (m, 2H), 0.92-0.84 (m, 2H), −0.02 (s, 9H).

Step B: N-(1,2,3,5,6,7-hexahydro-s-indacen-4-yl)-3-((3-methoxypropyl)sulfonyl)-1-((2-(trimethylsilyl)ethoxy)methyl)-1H-1,2,4-triazol-5-amine (79 mg, 0.156 mmol) was dissolved in HCl (4 M in dioxane, 3 mL, 12.00 mmol) and stirred at RT overnight. The reaction was concentrated in vacuo. The crude was purified by acidic prep HPLC (20-50% MeOH in water) to afford the title compound (13 mg, 22% yield) as a white solid.

LCMS m/z 377.7 (M+H)⁺ (ES⁺).

¹H NMR (DMSO-d₆) δ 13-19 (s, 1H), 8.99 (s, 1H), 6.97 (s, 1H), 3.38 (t, J=6.2 Hz, 2H), 3.36-3.32 (m, 2H), 3.20 (s, 3H), 2.83 (t, J=7.4 Hz, 4H), 2.64 (t, J=7.3 Hz, 4H), 1.98 (p, J=7.4 Hz, 4H), 1.90-1.82 (m, 2H).

### Example 66: N-(1,2,3,5,6,7-hexahydro-s-indacen-4-yl)-3-(((tetrahydrofuran-3-yl)methyl)sulfonyl)-1H-1,2,4-triazol-5-amine

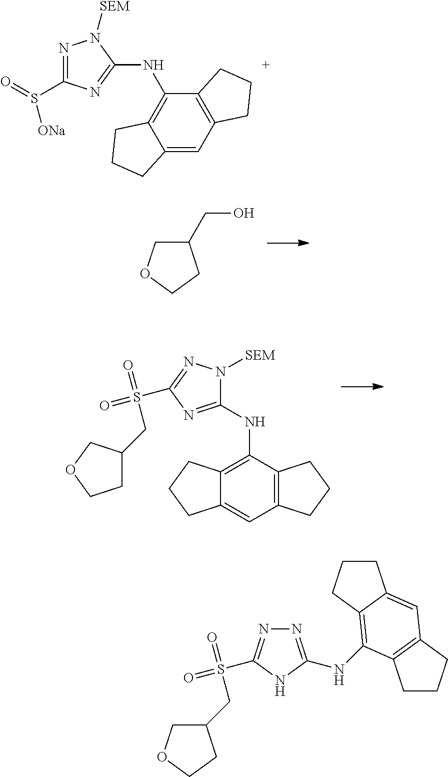

Prepared according to the general procedure of N-(1,2,3,5,6,7-hexahydro-s-indacen-4-yl)-3-((2-(1-methylpyrrolidin-2-yl)ethyl)sulfonyl)-1H-1,2,4-triazol-5-amine (Example 60) from sodium 5-((1,2,3,5,6,7-hexahydro-s-indacen-4-yl)amino)-1-((2-(trimethylsilyl)ethoxy)methyl)-1H-1,2,4-triazole-3-sulfinate (Intermediate B2) and (tetrahydrofuran-3-yl)methanol to afford the title compound (4 mg, 2% yield) as a white solid.

LCMS m/z 389.3 (M+H)⁺ (ES⁺).

¹H NMR (DMSO-d₆) δ 13.20 (s, 1H), 9.02 (s, 1H), 6.98 (s, 1H), 3.79 (dd, J=8.6, 7.2 Hz, 1H), 3.71 (td, J=8.3, 4.9 Hz, 1H), 3.64-3.59 (m, 1H), 3.54-3.43 (m, 2H), 2.83 (t, J=7.5 Hz, 4H), 2.64 (t, J=7.3 Hz, 4H), 2.58-2.52 (m, 1H), 2.07-2.01 (m, 1H), 1.97 (p, J=7.5 Hz, 4H), 1.69-1.60 (m, 1H). One proton obscured by solvent.

### Example 67: N-(1,2,3,5,6,7-hexahydro-s-indacen-4-yl)-3-(((tetrahydrofuran-2-yl)methyl)sulfonyl)-1H-1,2,4-triazol-5-amine

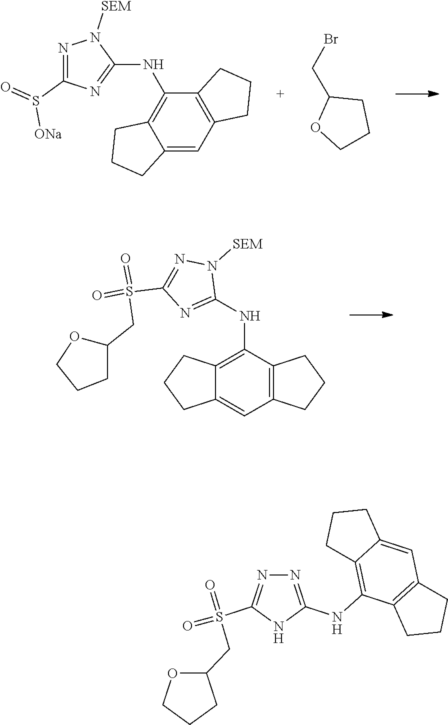

Prepared according to the general procedure of N-(1,2,3,5,6,7-hexahydro-s-indacen-4-yl)-3-((3-methoxypropyl)sulfonyl)-1H-1,2,4-triazol-5-amine (Example 65) from sodium 5-((1,2,3,5,6,7-hexahydro-s-indacen-4-yl)amino)-1-((2-(trimethylsilyl)ethoxy)-methyl)-1H-1,2,4-triazole-3-sulfinate (Intermediate B2) and 2-(bromomethyl)-tetrahydrofuran to afford the title compound (6 mg, 3% yield) as a white powder.

LCMS m/z 389.5 (M+H)⁺ (ES⁺); 387.2 (M−H)⁻ (ES⁻).

¹H NMR (DMSO-d₆) δ 13.12 (s, 1H), 8.96 (s, 1H), 6.96 (s, 1H), 4.18 (app p, J=6.3 Hz, 1H), 3.71-3.48 (m, 4H), 2.82 (t, J=7.5 Hz, 4H), 2.63 (t, J=7.3 Hz, 4H), 2.04-1.91 (m, 5H), 1.89-1.72 (m, 2H), 1.67-1.58 (m, 1H).

### Example 68:2-((5-((1,2,3,5,6,7-hexahydro-s-indacen-4-yl)amino)-4H-1,2,4-triazol-3-yl)sulfonyl)-N-methylacetamide

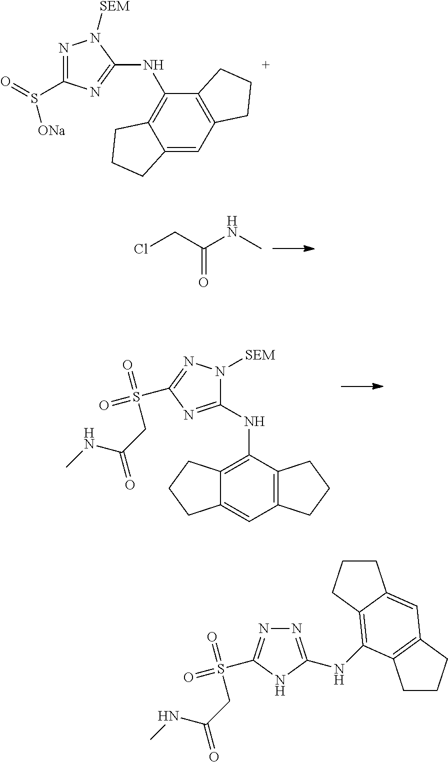

Prepared according to the general procedure of N-(1,2,3,5,6,7-hexahydro-s-indacen-4-yl)-3-((morpholin-2-ylmethyl)sulfonyl)-1H-1,2,4-triazol-5-amine (Example 57) from sodium 5-((1,2,3,5,6,7-hexahydro-s-indacen-4-yl)amino)-1-((2-(trimethylsilyl)ethoxy)-methyl)-1H-1,2,4-triazole-3-sulfinate (Intermediate B2) and 2-chloro-N-methylacetamide to afford the title compound (9 mg, 5% yield) as a white solid.

LCMS m/z 376.2 (M+H)⁺ (ES⁺).

¹H NMR (DMSO-d₆) δ13.16 (s, 1H), 8.97 (s, 1H), 8.22 (q, J=4.7 Hz, 1H), 6.97 (s, 1H), 4.23 (s, 2H), 2.83 (t, J=7.4 Hz, 4H), 2.64 (t, J=7.4 Hz, 4H), 2.60 (d, J=4.6 Hz, 3H), 1.98 (p, J=7.4 Hz, 4H).

### Example 6A: 1-(1-((5-(4-fluoro-2-isopropyl-6-(2-methoxypyridin-4-yl)phenoxy)-4H-1,2,4-triazol-3-yl)sulfonyl)pyrrolidin-3-yl)-N,N-dimethylmethanamine

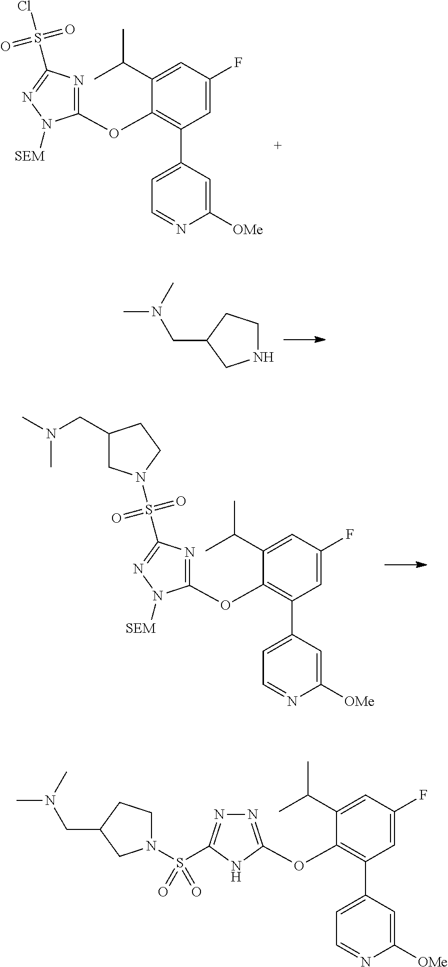

Step A: To a solution of 5-(4-fluoro-2-isopropyl-6-(2-methoxypyridin-4-yl)phenoxy)-1-((2-(trimethylsilyl)ethoxy)methyl)-1H-1,2,4-triazole-3-sulfonyl chloride (Intermediate B7) (100 mg, 179.86 μmol, 1 eq) in DCM (3 mL) was added N,N-dimethyl-1-(pyrrolidin-3-yl)-methanamine (28 mg, 215.83 μmol, 1.2 eq). Then the reaction mixture was stirred at 25° C. for 30 minutes. The mixture was concentrated in vacuum. The residue was purified by silica gel column chromatography (SiO₂, petroleum ether:ethyl acetate, 1:1 to 0:1) to give 1-(1-((5-(4-fluoro-2-isopropyl-6-(2-methoxypyridin-4-yl)phenoxy)-1-((2-(trimethylsilyl)ethoxy)methyl)-1H-1,2,4-triazol-3-yl)sulfonyl)pyrrolidin-3-yl)-N,N-dimethylmethanamine (0.1 g, yield over three steps: 95.6%, 91% purity on LCMS) as a yellow oil.

¹H NMR (400 MHz, CDCl₃) δ 8.11 (d, 1H), 7.11 (dd, 1H), 6.93 (dd, 1H), 6.87 (dd, 1H), 6.71 (s, 1H), 5.33 (s, 2H), 3.92 (s, 3H), 3.64 (t, 2H), 3.51-349 (m, 1H), 3.41-3.38 (m, 1H), 3.23-3.17 (m, 1H), 3.09-3.06 (m, 1H), 3.01-2.99 (m, 1H), 2.34-2.24 (m, 3H), 2.19 (s, 6H), 1.91-1.87 (m, 1H), 1.54-149 (m, 1H), 1.24 (d, 6H), 0.95-0.90 (m, 2H), 0.02 (s, 9H).

LCMS: m/z 649.3 (M+H)⁺ (ES⁺).

Step B: To a solution of 1-(1-((5-(4-fluoro-2-isopropyl-6-(2-methoxypyridin-4-yl)-phenoxy)-1-((2-(trimethylsilyl)ethoxy)methyl)-1H-1,2,4-triazol-3-yl)sulfonyl)-pyrrolidin-3-yl)-N,N-dimethylmethanamine (100 mg, 154.32 μmol, 1 eq) in DCM (4 mL) was added TFA (4 mL). Then the solution was stirred at 25° C. for 2 hours. The reaction mixture was concentrated in vacuum. The residue was purified by prep-HPLC (column: Waters Xbridge C18, 150 mm*50 mm*10 μm; mobile phase: [A: water (0.05% ammonia hydroxide v/v), B: MeCN]; B %: 8%-38%,11.5 minutes) to give the title compound (31.54 mg, 39.41% yield, 100% purity on LCMS) as a white solid.

¹H NMR (400 MHz, CDCl₃) δ 8.08 (d, 1H), 7.06 (dd, 1H), 7.01 (dd, 1H), 6.91 (dd, 1H), 6.82 (s, 1H), 3.89 (s, 3H), 3.65-3.60 (m, 1H), 3.46-3.39 (m, 1H), 3.29-3.22 (m, 2H), 2.87-2.82 (m, 1H), 2.72-2.66 (m, 1H), 2.50-2.46 (m, 1H), 2.44 (s, 6H), 2.35-2.34 (m, 1H), 1.94-1.90 (m, 1H), 1.47-1.42 (m, 1H) and 1.18 (d, 6H). One exchangeable proton not observed.

LCMS: m/z 519.2 (M+H)⁺ (ES⁺).

### Example 70: N-(1,2,3,5,6,7-hexahydro-s-indacen-4-yl)-3-(((3-methyl-1,2,4-oxadiazol-5-yl)methyl)sulfonyl)-1H-1,2,4-triazol-5-amine

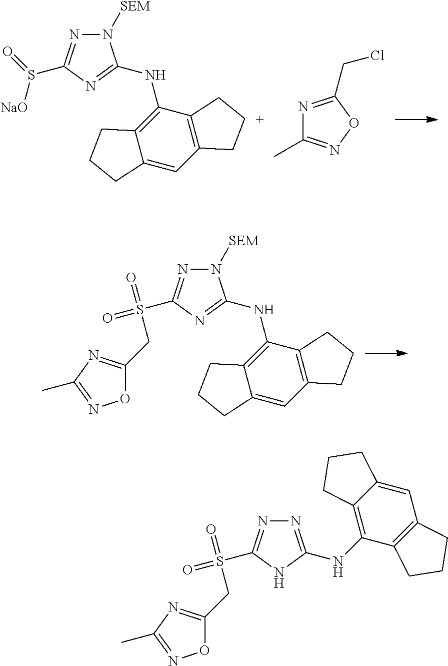

Prepared according to the general procedure of N-(1,2,3,5,6,7-hexahydro-s-indacen-4-yl)-3-((2-(1-methylpiperidin-2-yl)ethyl)sulfonyl)-1H-1,2,4-triazol-5-amine (Example 59) from sodium 5-((1,2,3,5,6,7-hexahydro-s-indacen-4-yl)amino)-1-((2-(trimethylsilyl)ethoxy)methyl)-1H-1,2,4-triazole-3-sulfinate (Intermediate B2) and 5-(chloromethyl)-3-methyl-1,2,4-oxadiazole to afford the title compound (75 mg, 42% yield) as a white solid.

LCMS m/z 401.1 (M+H)⁺ (ES⁺); 399.2 (M−H)⁻ (ES⁻).

¹H NMR (DMSO-d₆) δ 13.30 (s, 1H), 9.04 (s, 1H), 6.97 (s, 1H), 5.31 (s, 2H), 2.83 (t, J=7.3 Hz, 4H), 2.63 (t, J=7.4 Hz, 4H), 2.33 (s, 3H), 1.98 (p, J=7.4 Hz, 4H).

### Example 71: N-(1,2,3,5,6,7-hexahydro-s-indacen-4-yl)-3-((pyridin-2-ylmethyl)sulfonyl)-1H-1,2,4-triazol-5-amine

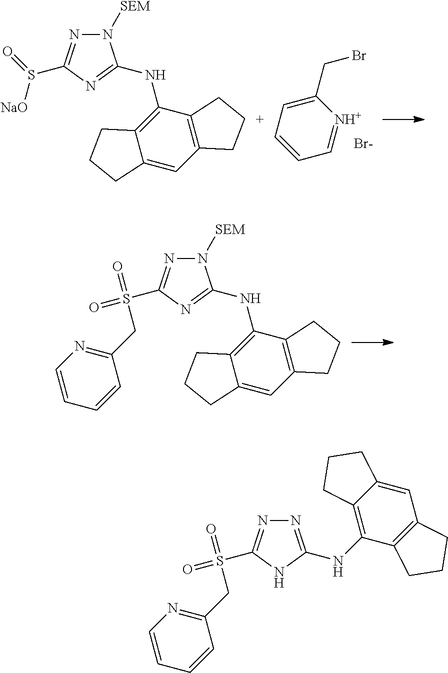

Sodium 5-((1,2,3,5,6,7-hexahydro-s-indacen-4-yl)amino)-1-((2-(trimethylsilyl)ethoxy)-methyl)-1H-1,2,4-triazole-3-sulfinate (Intermediate B2) (100 mg, 0.219 mmol), 2-(bromomethyl)pyridin-1-ium bromide (55 mg, 0.217 mmol), KI (5 mg, 0.030 mmol) and Cs₂CO₃ (71 mg, 0.218 mmol) were dissolved in DMSO (2 mL) and stirred at RT for 1 h. The reaction was diluted with aq 2 M NaOH (20 mL), extracted with DCM (2×20 mL). The organics were dried (phase separator) and concentrated in vacuo. The resulting residue was dissolved in HCl (4 M in dioxane, 4 mL, 16.00 mmol) and stirred at RT for 16 h. The reaction was concentrated in vacuo and the product was purified by acidic prep HPLC (50-80% MeOH in water) to afford the title compound (18 mg, 21% yield) as a white powder.

LCMS m/z 396.3 (M+H)⁺ (ES⁺); 393.8 (M−H)⁻ (ES⁻).

¹H NMR (DMSO-d₆) δ 13.14 (s, 1H), 8.95 (s, 1H), 8.49 (m, 1H), 7.80 (td, J=7.7, 1.9 Hz, 1H), 7.41-7.36 (m, 2H), 6.97 (s, 1H), 4.81 (s, 2H), 2.83 (t, J=7.4 Hz, 4H), 2.63 (t, J=7.5 Hz, 4H), 1.99 (p, J=7.3 Hz, 4H).

### Example 72: N-(1,2,3,5,6,7-hexahydro-s-indacen-4-yl)-3-((pyridin-4-ylmethyl)sulfonyl)-1H-1,2,4-triazol-5-amine

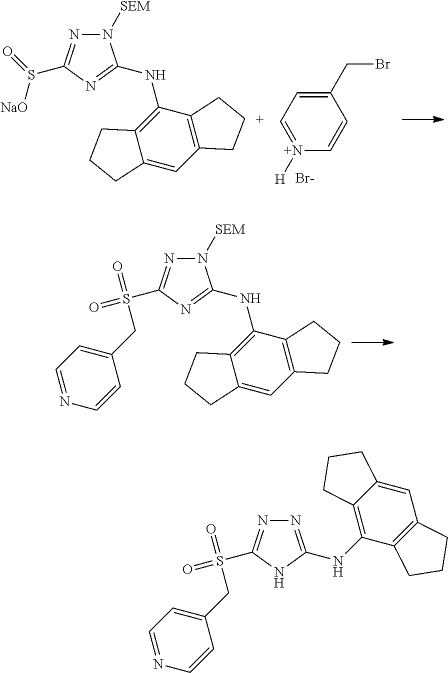

Prepared according to the general procedure of N-(1,2,3,5,6,7-hexahydro-s-indacen-4-yl)-3-((2-(1-methylpiperidin-2-yl)ethyl)sulfonyl)-1H-1,2,4-triazol-5-amine (Example 59) from sodium 5-((1,2,3,5,6,7-hexahydro-s-indacen-4-yl)amino)-1-((2-(trimethylsilyl)ethoxy)methyl)-1H-1,2,4-triazole-3-sulfinate (Intermediate B2) and 4-(bromomethyl)pyridin-1-ium bromide to afford the title compound (19 mg, 11% yield) as a light orange powder.

LCMS m/z 396.0 (M+H)⁺ (ES⁺); 394.3 (M−H)⁻ (ES⁻).

¹H NMR (DMSO-d₆) δ 13.17 (s, 1H), 9.01 (s, 1H), 8.55-8.53 (m, 2H), 7.28-7.27 (m, 2H), 6.97 (s, 1H), 4.79 (s, 2H), 2.83 (t, J=7.4 Hz, 4H), 2.61 (t, J=7.3 Hz, 4H), 1.98 (p, J=7.3 Hz, 4H).

### Example 73: N-(1,2,3,5,6,7-hexahydro-s-indacen-4-yl)-3-((pyridin-3-ylmethyl)sulfonyl)-1H-1,2,4-triazol-5-amine

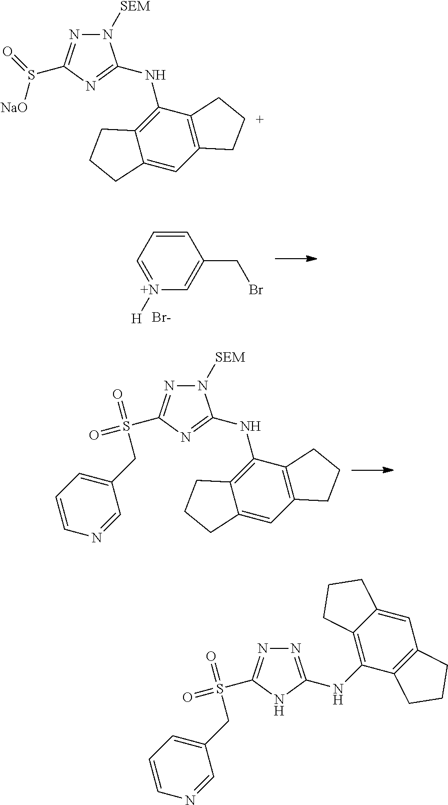

Prepared according to the general procedure of N-(1,2,3,5,6,7-hexahydro-s-indacen-4-yl)-3-((2-(1-methylpiperidin-2-yl)ethyl)sulfonyl)-1H-1,2,4-triazol-5-amine (Example 59) from sodium 5-((1,2,3,5,6,7-hexahydro-s-indacen-4-yl)amino)-1-((2-(trimethylsilyl)ethoxy)methyl)-1H-1,2,4-triazole-3-sulfinate (Intermediate B2) and 3-(bromomethyl)pyridin-1-ium bromide to afford the title compound (35 mg, 20% yield) as a light orange powder.

LCMS m/z 396.0 (M+H)⁺ (ES⁺); 394.3 (M−H)⁻ (ES⁻).

¹H NMR (DMSO-d₆) δ 13-19 (s, 1H), 8.97 (s, 1H), 8.53 (dd, J=4.7, 1.6 Hz, 1H), 8.40 (d, J=2.2 Hz, 1H), 7.70-7.66 (m, 1H), 6.98-6.94 (m, 1H), 6.97 (s, 1H), 4.77 (s, 2H), 2.83 (t, J=7.3 Hz, 4H), 2.61 (t, J=7.4 Hz, 4H), 1.98 (p, J=7.3 Hz, 4H).

### Example 74: 3-((3-(dimethylamino)benzyl)sulfonyl)-N-(1,2,3,5,6,7-hexahydro-s-indacen-4-yl)-1H-1,2,4-triazol-5-amine

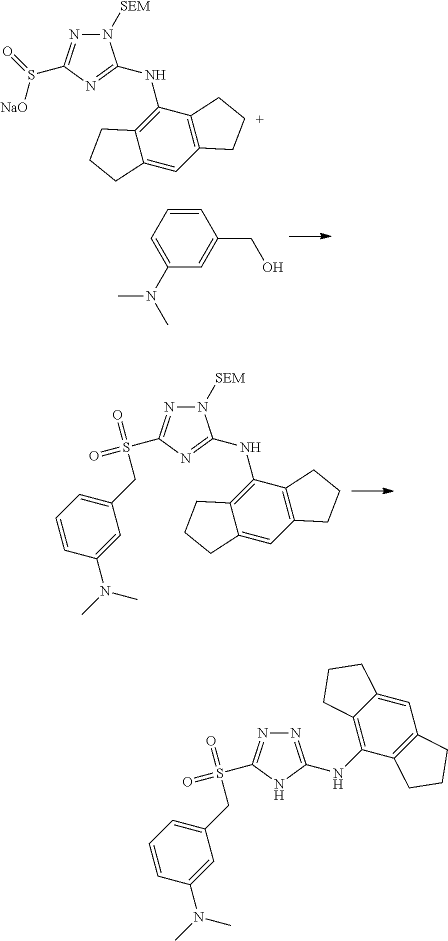

Prepared according to the general procedure of N-(1,2,3,5,6,7-hexahydro-s-indacen-4-yl)-3-(((1-methylpiperidin-3-yl)methyl)sulfonyl)-1H-1,2,4-triazol-5-amine (Example 58) from sodium 5-((1,2,3,5,6,7-hexahydro-s-indacen-4-yl)amino)-1-((2-(trimethylsilyl)ethoxy)methyl)-1H-1,2,4-triazole-3-sulfinate (Intermediate B2) and (3-(dimethylamino)phenyl)methanol to afford the title compound (16 mg, 8% yield) as a white powder.

LCMS m/z 438.4 (M+H)⁺ (ES⁺); 436.3 (M−H)⁻ (ES⁻).

¹H NMR (DMSO-d₆) δ 13.13 (s, 1H), 8.91 (s, 1H), 7.11 (t, J=7.8 Hz, 1H), 6.98-6.93 (m, 1H), 6.68 (dd, J=8.3, 2.2 Hz, 1H), 6.54 (d, J=7.5 Hz, 1H), 6.51-6.48 (m, 1H), 4.54 (s, 2H), 2.88-2.78 (m nioH), 2.61 (t, J=7.4 Hz, 4H), 1.97 (p, J=7.4 Hz, 4H).

### Example 75: N-(1,2,3,5,6,7-hexahydro-s-indacen-4-yl)-5-(methylsulfonyl)-4H-1,2,4-triazol-3-amine

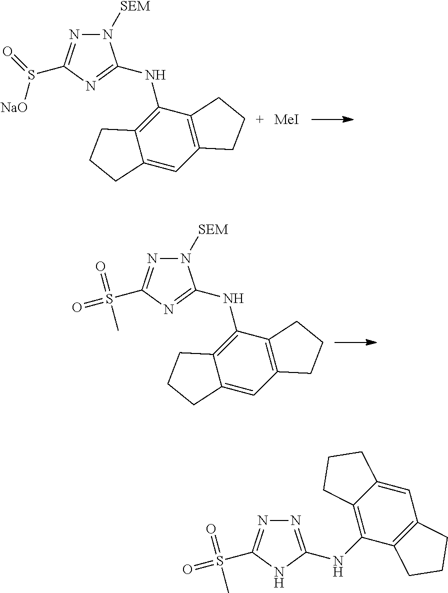

Sodium 5-((1,2,3,5,6,7-hexahydro-s-indacen-4-yl)amino)-1-((2-(trimethylsilyl)ethoxy)-methyl)-1H-1,2,4-triazole-3-sulfinate (Intermediate B2) (70 mg, 0.153 mmol) was dissolved in DMSO (1 mL) and MeI (0.014 mL, 0.230 mmol) was added. The reaction was stirred at RT overnight and the mixture was diluted with water (4 mL) and extracted with EtOAc (2×4 mL). The combined organics were washed with brine (10 mL), dried (phase separator) and concentrated in vacuo. TFA (1 mL) was added to the residue. The reaction was stirred at RT for 1 h and concentrated in vacuo. The product was purified by basic preparative HPLC (5-50% MeCN in water) to afford the title compound (8.3 mg, 17% yield) as a pale white solid.

LCMS m/z 319.0 (M+H)⁺ (ES⁺); 317.0 (M−H)⁻ (ES⁻).

¹H NMR (DMSO-d₆) δ 8.88 (s, 1H), 6.96 (s, 1H), 3.23 (s, 3H), 2.83 (t, J=7.4 Hz, 4H), 2.64 (t, J=7.3 Hz, 4H), 1.98 (p, J=7.4 Hz, 4H). One exchangeable proton not observed.

### Example 76:5-((3-(diethylamino)propyl)sulfonyl)-N-(5-(2-methoxypyridin-4-yl)-2,3-dihydro-1H-inden-4-yl)-4H-1,2,4-triazol-3-amine

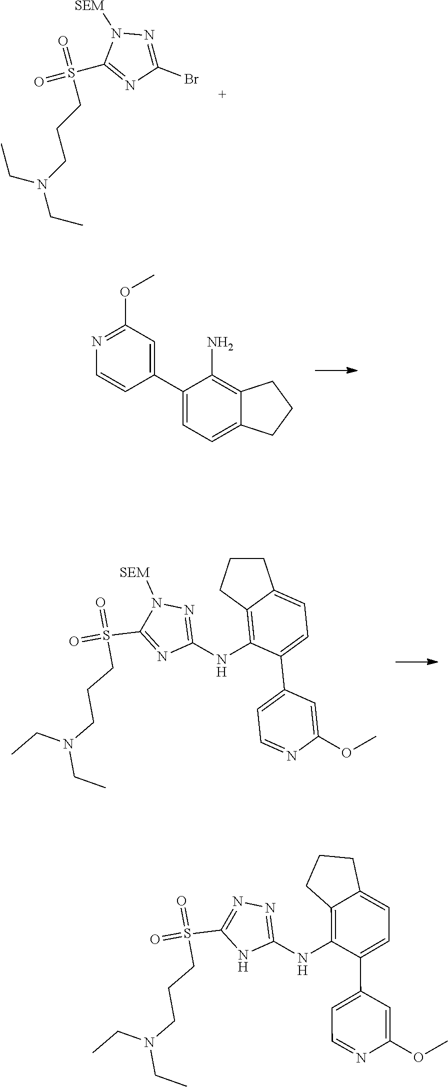

Prepared according to the general procedure of N-(7-fluoro-5-(pyridin-3-yl)-2,3-dihydro-1H-inden-4-yl)-5-(phenylsulfonyl)-1H-1,2,4-triazol-3-amine (Example 41) from 3-((3-bromo-1-((2-(trimethylsilyl)ethoxy)methyl)-1H-1,2,4-triazol-5-yl)sulfonyl)-N,N-diethylpropan-1-amine (Intermediate A7) and 5-(2-methoxypyridin-4-yl)-2,3-dihydro-1H-inden-4-amine (Intermediate R4) to afford the title compound (36 mg, 11% yield) as a white solid.

LCMS m/z 485.3 (M+H)⁺ (ES⁺).

¹H NMR (DMSO-d₆) δ 8.93 (s, 1H), 8.09 (d, J=5.3 Hz, 1H), 7.26 (d, J=7.7 Hz, 1H), 7.19 (d, J=7.7 Hz, 1H), 6.91 (dd, J=5.3, 1.4 Hz, 1H), 6.73 (app br s, 1H), 3.92 (s, 3H), 3.25-3.21 (m, 2H), 2.95 (t, J=7.4 Hz, 2H), 2.69 (t, J=7.4 Hz, 2H), 2.43-2.37 (m, 6H), 2.02 (p, J=7.5 Hz, 2H), 1.66-1.60 (m, 2H), 0.90 (t, J=7.1 Hz, 6H). One exchangeable proton not observed.

### Example 77:5-((3-(diethylamino)propyl)sulfonyl)-N-(7-fluoro-5-(pyridin-4-yl)-2,3-dihydro-1H-inden-4-yl)-4H-1,2,4-triazol-3-amine

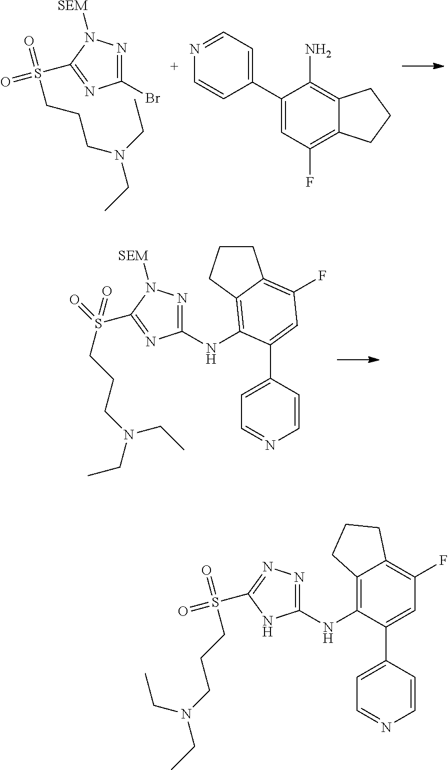 [SMILES: CCN(CC)CCCS(=O)(=O)c1nc(Br)nn1COCC[Si](C)(C)C ; Nc1c(-c2ccncc2)cc(F)c2c1CCC2 ; CCN(CC)CCCS(=O)(=O)c1nc(Nc2c(-c3ccncc3)cc(F)c3c2CCC3)nn1COCC[Si](C)(C)C ; CCN(CC)CCCS(=O)(=O)c1nnc(Nc2c(-c3ccncc3)cc(F)c3c2CCC3)[nH]1]

Prepared according to the general procedure of N-(7-fluoro-5-(pyridin-3-yl)-2,3-dihydro-1H-inden-4-yl)-5-(phenylsulfonyl)-1H-1,2,4-triazol-3-amine (Example 41) from 3-((3-bromo-1-((2-(trimethylsilyl)ethoxy)methyl)-1H-1,2,4-triazol-5-yl)sulfonyl)-N,N-diethylpropan-1-amine (Intermediate A7) and 7-fluoro-5-(pyridin-4-yl)-2,3-dihydro-1H-inden-4-amine (Intermediate R7) to afford the title compound (130 mg, 44% yield) as an off-white solid.

LCMS m/z 473.2 (M+H)⁺ (ES⁺).

¹H NMR (DMSO-d₆) δ 8.98 (s, 1H), 8.54 (d, J=5.0 Hz, 2H), 7.35-7.34 (m, 2H), 7.13 (d, J=9.1 Hz, 1H), 3.25-3.22 (m, 2H), 3.00 (t, J=7.5 Hz, 2H), 2.76 (t, J=7.5 Hz, 2H), 2.43 (m, 6H), 2.10 (p, J=7.5 Hz, 2H), 1.64 (p, J=7.1 Hz, 2H), 0.92 (t, J=7.1 Hz, 6H). One exchangeable proton not observed.

### Example 78: 5-((3-(diethylamino)propyl)sulfonyl)-N-(7-fluoro-5-phenyl-2,3-dihydro-1H-inden-4-yl)-4H-1,2,4-triazol-3-amine

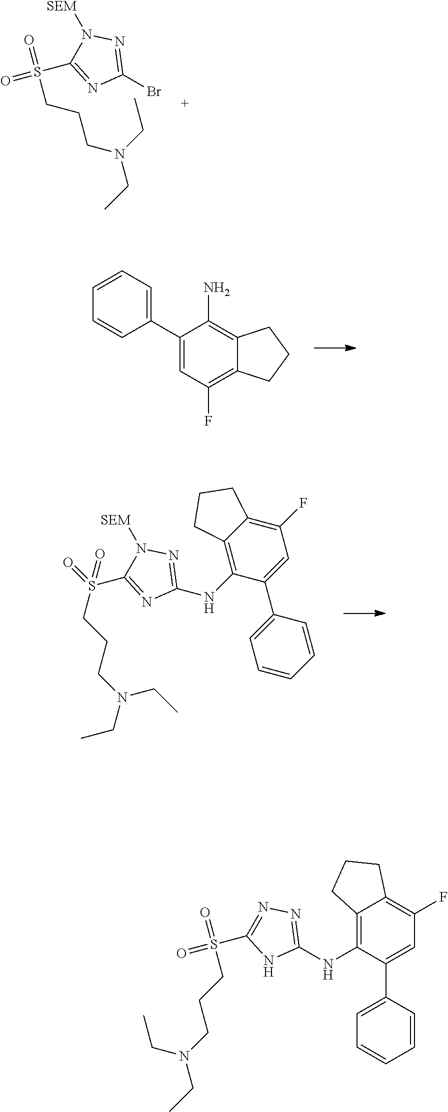 [SMILES: CCN(CC)CCCS(=O)(=O)c1nc(Br)nn1COCC[Si](C)(C)C ; Nc1c(-c2ccccc2)cc(F)c2c1CCC2 ; CCN(CC)CCCS(=O)(=O)c1nc(Nc2c(-c3ccccc3)cc(F)c3c2CCC3)nn1COCC[Si](C)(C)C ; CCN(CC)CCCS(=O)(=O)c1nnc(Nc2c(-c3ccccc3)cc(F)c3c2CCC3)[nH]1]

Prepared according to the general procedure of N-(7-fluoro-5-(pyridin-3-yl)-2,3-dihydro-1H-inden-4-yl)-5-(phenylsulfonyl)-1H-1,2,4-triazol-3-amine (Example 41) from 3-((3-bromo-1-((2-(trimethylsilyl)ethoxy)methyl)-1H-1,2,4-triazol-5-yl)sulfonyl)-N,N-diethylpropan-1-amine (Intermediate A7) and 7-fluoro-5-phenyl-2,3-dihydro-1H-inden-4-amine (Intermediate C1) to afford the title compound (152 mg, 50% yield) as yellow solid.

LCMS m/z 472.2 (M+H)⁺ (ES⁺).

¹H NMR (DMSO-d₆) δ 12.93 (br s, 1H), 8.83 (s, 1H), 7.36-7.27 (m, 5H), 7.01 (d, J=9.3 Hz, 1H), 3.24-3.21 (m, 2H), 2.98 (t, J=7.5 Hz, 2H), 2.74 (t, J=7.5 Hz, 2H), 2.42-2.37 (m, 6H), 2.08 (p, J=7.5 Hz, 2H), 1.62 (p, J=7.1 Hz, 2H), 0.90 (t, J=7.1 Hz, 6H).

### Example 70: 5-((3-(diethylamino)propyl)sulfonyl)-N-(4-fluoro-2-isopropyl-6-(2-methoxypyridin-4-yl)phenyl)-4H-1,2,4-triazol-3-amine

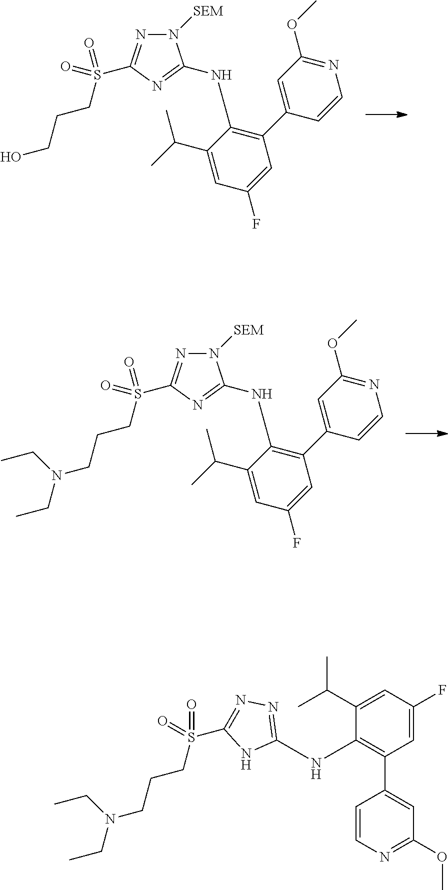 [SMILES: COc1cc(-c2cc(F)cc(C(C)C)c2Nc2nc(S(=O)(=O)CCCO)nn2COCC[Si](C)(C)C)ccn1 ; CCN(CC)CCCS(=O)(=O)c1nc(Nc2c(-c3ccnc(OC)c3)cc(F)cc2C(C)C)n(COCC[Si](C)(C)C)n1 ; CCN(CC)CCCS(=O)(=O)c1nnc(Nc2c(-c3ccnc(OC)c3)cc(F)cc2C(C)C)[nH]1]

Prepared according to the general procedure of 3-((3-bromo-1-((2-(trimethylsilyl)-ethoxy)methyl)-1H-1,2,4-triazol-5-yl)sulfonyl)-N,N-diethylpropan-1-amine (Intermediate A7) from 3-((5-((4-fluoro-2-isopropyl-6-(2-methoxypyridin-4-yl)phenyl)amino)-1-((2-(trimethylsilyl)ethoxy)methyl)-1H-1,2,4-triazol-3-yl)sulfonyl)propan-1-ol (Intermediate B3) and diethylamine followed by SEM-deprotection using TFA according to the general procedure of N-(1,2,3,5,6,7-hexahydro-s-indacen-4-yl)-5-(phenylsulfonyl)-1H-1,2,4-triazol-3-amine (Example 40, Step B) to afford the title compound (46 mg, 17% yield over 2 steps) as a white solid.

LCMS m/z 505.4 (M+H)⁺ (ES⁺); 503.4 (M−H)⁻ (ES⁻).

¹H NMR (Acetonitrile-d₃) δ 8.30 (s, 1H), 8.05 (d, J=5.3 Hz, 1H), 7.24 (dd, J=10.0, 3.0 Hz, 1H), 7.04 (dd, J=8.8, 3.0 Hz, 1H), 6.91 (dd, J=5.3, 1.5 Hz, 1H), 6.81-6.68 (m, 1H), 3.86 (s, 3H), 3.31-3.23 (m, 1H), 3.19 (t, J=7.3 Hz, 2H), 3.10-2.96 (m, 6H), 1.21-1.13 (m, 12H). One exchangable proton not observed, 2 aliphatic protons obscured by solvent.

### Example 80: N-(2-(diethylamino)ethyl)-3-((1,2,3,5,6,7-hexahydro-s-indacen-4-yl)amino)-N-methyl-1H-1,2,4-triazole-5-sulfonamide

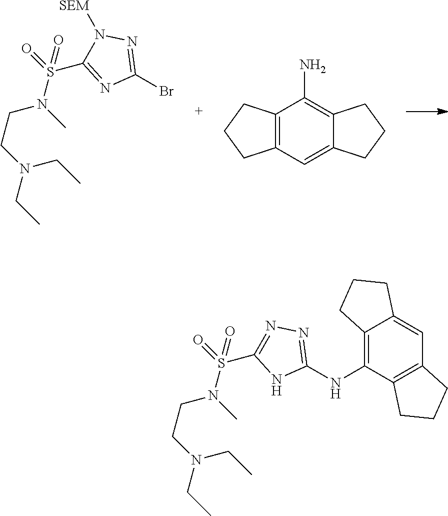 [SMILES: CCN(CC)CCN(C)S(=O)(=O)c1nc(Br)nn1COCC[Si](C)(C)C ; Nc1c2c(cc3c1CCC3)CCC2 ; CCN(CC)CCN(C)S(=O)(=O)c1nnc(Nc2c3c(cc4c2CCC4)CCC3)[nH]1]

Pd-175 (28.2 mg, 0.036 mmol), ground K₂CO₃ (80 mg, 0.578 mmol), 1,2,3,5,6,7-hexahydro-s-indacen-4-amine (68.9 mg, 0.397 mmol) and 3-bromo-N-(2-(diethylamino)ethyl)-N-methyl-1-((2-(trimethylsilyl)ethoxy)methyl)-1H-1,2,4-triazole-5-sulfonamide (Intermediate A8) (170 mg, 0.361 mmol) were placed in a reaction vessel under N₂. 1,4-Dioxane (8 mL) was added then the reaction was deoxygenated with a stream of N₂ for 10 min and stirred at 65° C. for 19 h. After cooling to RT, the reaction was diluted with EtOAc (100 mL) and washed with water (100 mL). The organics were dried (MgSO₄) and concentrated in vacuo. The crude mixture was passed through SCX (12 g) eluting with DCM/MeOH (1:1, 50 mL) then 0.7 N NH₃ in MeOH (70 mL). The ammoniacal solution was concentrated in vacuo. The product was purified by chromatography on silica gel (24 g column, 0-10% MeOH/DCM) to afford the title compound (46 mg, 29% yield) as a brown solid.

LCMS m/z 433.3 (M+H)⁺ (ES⁺); 431.2 (M−H)⁻ (ES⁻).

¹H NMR (DMSO-d₆) δ 12.94 (s, 1H), 8.89 (s, 1H), 6.95 (s, 1H), 3.16 (t, J=7.2 Hz, 2H), 2.85 (s, 3H), 2.82 (t, J=7.4 Hz, 4H), 2.63 (t, J=7.3 Hz, 4H), 2.54 (t, J=7.2 Hz, 2H), 2.46 (q, J=7.1 Hz, 4H), 1.97 (p, J=7.4 Hz, 4H), 0.93 (t, J=7.1 Hz, 6H).

### Example 81: N-(2-(diethylamino)ethyl)-5-((1,2,3,5,6,7-hexahydro-s-indacen-4-yl)amino)-4H-1,2,4-triazole-3-sulfonamide

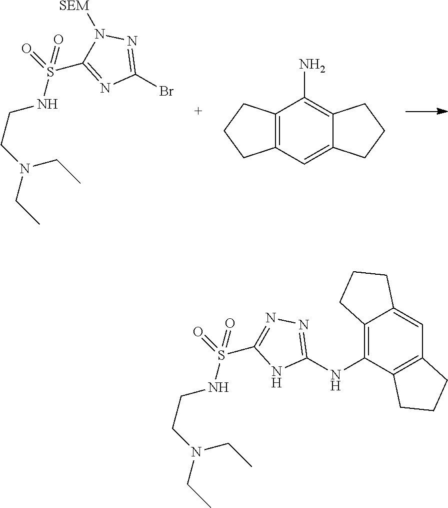 [SMILES: CCN(CC)CCNS(=O)(=O)c1nc(Br)nn1COCC[Si](C)(C)C ; Nc1c2c(cc3c1CCC3)CCC2 ; CCN(CC)CCNS(=O)(=O)c1nnc(Nc2c3c(cc4c2CCC4)CCC3)[nH]1]

Pd-175 (30.0 mg, 0.038 mmol), ground K₂CO₃ (85 mg, 0.613 mmol), 1,2,3,5,6,7-hexahydro-s-indacen-4-amine (73.1 mg, 0.422 mmol) and 3-bromo-N-(2-(diethylamino)ethyl)-1-((2-(trimethylsilyl)ethoxy)methyl)-1H-1,2,4-triazole-5-sulfonamide (Intermediate A9) (175 mg, 0.383 mmol) were placed in a reaction vessel under N₂. 1,4-Dioxane (10 mL) was added, the reaction was deoxygenated with a stream of N₂ for 10 min, then stirred at 65° C. for 18 h. The crude mixture was diluted with MeCN (12 mL) and water (2 mL). SCX (5 g) was added and the resulting slurry stirred for 1.5 h. The mixture was filtered, rinsed with MeOH/DCM (1:1, 40 mL) and the product eluted with 0.7 M NH₃ in MeOH (70 mL). The ammoniacal solution was concentrated in vacuo and the product was purified by chromatography on silica gel (24 g column, 0-10% (0.7 M ammonia/MeOH)/DCM) to afford the title compound (45 mg, 27% yield) as a pale yellow solid.

LCMS m/z 419.3 (M+H)⁺ (ES⁺); 417.1 (M−H)⁻ (ES⁻).

¹H NMR (DMSO-d₆) δ 12.84 (s, 1H), 8.86 (s, 1H), 7.71 (s, 1H), 7.00 (s, 1H), 3.04 (t, J=7.5 Hz, 2H), 2.87 (t, J=7.4 Hz, 4H), 2.69 (t, J=7.3 Hz, 4H), 2.52-2.50 (m, 4H), 2.02 (p, J=7.4 Hz, 4H), 0.96 (t, J=7.1 Hz, 6H). Two protons obscured by solvent.

### Example 82: 5-((1,2,3,5,6,7-hexahydro-s-indacen-4-yl)amino)-N-isopentyl-1H-1,2,4-triazole-3-sulfonamide

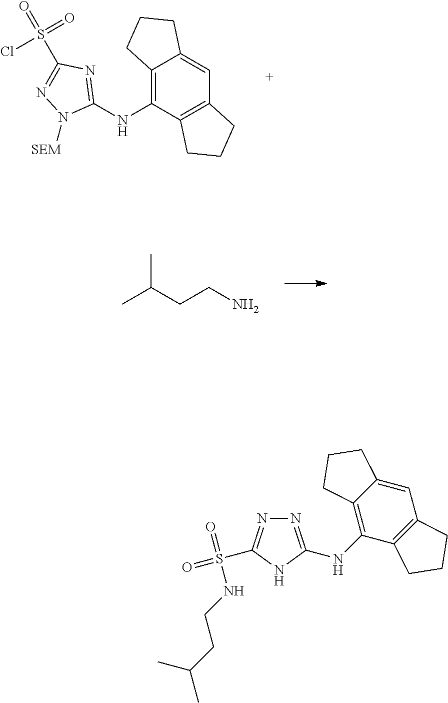

Et₃N (0.045 mL, 0.320 mmol) and 3-methylbutan-1-amine (0.037 ml, 0.320 mmol) were added to 5-((1,2,3,5,6,7-hexahydro-s-indacen-4-yl)amino)-1-((2-(trimethylsilyl)-ethoxy)methyl)-1H-1,2,4-triazole-3-sulfonyl chloride (Intermediate B4) (100 mg, 0.213 mmol) in DCM (4 mL) at 0° C. The reaction was stirred for 1 h and concentrated in vacuo. TFA (1 mL) was added and the reaction was stirred at RT for 1 h. The product was purified by basic prep HPLC (5-50% MeCN in water) to afford the title compound (1.8 mg, 2% yield) as a pale white solid.

LCMS m/z 390.2 (M+H)⁺ (ES⁺); 388.2 (M−H)⁻ (ES⁻).

¹H NMR (DMSO-d₆) δ 8.77 (s, 1H), 7.72 (s, 1H), 6.94 (s, 1H), 2.94 (t, J=7.4 Hz, 2H), 2.82 (t, J=7.4 Hz, 4H), 2.64 (t, J=7.3 Hz, 4H), 1.97 (p, J=7.4 Hz, 4H), 1.63-1.51 (m, 1H), 1.29 (q, J=7.2 Hz, 2H), 0.82 (d, J=6.6 Hz, 6H). One exchangeable proton not observed.

### Example 83: 5-((1,2,3,5,6,7-hexahydro-s-indacen-4-yl)amino)-N-(2-methoxyethyl)-4H-1,2,4-triazole-3-sulfonamide

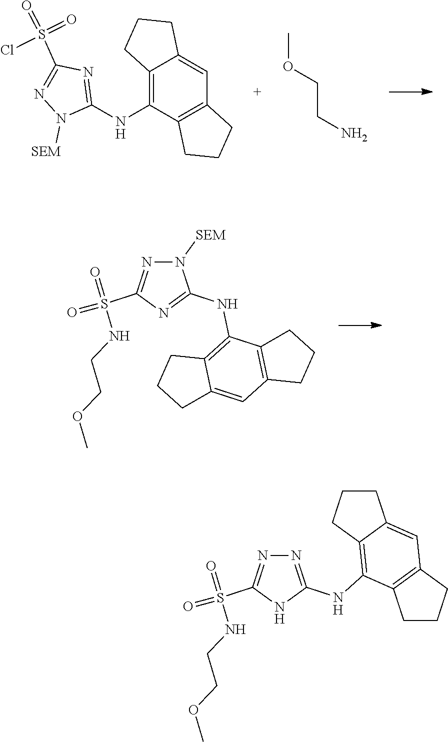

Step A: 5-((1,2,3,5,6,7-Hexahydro-s-indacen-4-yl)amino)-1-((2-(trimethylsilyl)ethoxy)-methyl)-1H-1,2,4-triazole-3-sulfonyl chloride (Intermediate B4) (500 mg, 1.066 mmol) in DCM (12 mL) was added to a cooled solution of 2-methoxyethanamine (0.185 mL, 2.13 mmol), triethylamine (0.297 mL, 2.13 mmol) and DCM (5 mL). The reaction was warmed to RT and stirred for 1 h. The crude reaction mixture was concentrated onto silica and purified by chromatography on silica gel (12 g column, 0-100% (0.7 M Ammonia/MeOH)/DCM) to afford 5-((1,2,3,5,6,7-hexahydro-s-indacen-4-yl)amino)-N-(2-methoxyethyl)-1-((2-(trimethylsilyl)ethoxy)methyl)-1H-1,2,4-triazole-3-sulfonamide (279.2 mg, 49% yield) as a sticky yellow oil.

LCMS m/z 508.3 (M+H)⁺ (ES⁺).

¹H NMR (DMSO-d₆) δ 8.75 (s, 1H), 8.05 (t, J=5-7 Hz, 1H), 6.97 (s, 1H), 5.50 (s, 2H), 3.66-3.59 (m, 2H), 3.30 (t, J=6.0 Hz, 2H), 3.18 (s, 3H), 3.01 (q, J=5.9 Hz, 2H), 2.83 (t, J=7.2 Hz, 4H), 2.66 (t, J=7.3 Hz, 4H), 1.97 (p, J=7.4 Hz, 4H), 0.92-0.86 (m, 2H), −0.02 (s, 9H).

Step B: 5-((1,2,3,5,6,7-Hexahydro-s-indacen-4-yl)amino)-N-(2-methoxyethyl)-1-((2-(trimethylsilyl)ethoxy)methyl)-1H-1,2,4-triazole-3-sulfonamide (237.8 mg, 0.468 mmol) was dissolved in HCl (4 M in dioxane, 6 mL, 24.0 mmol) and stirred at RT overnight. The reaction mixture was concentrated in vacuo. The crude product was purified by acidic prep HPLC (35-65% MeOH in water) to afford the title compound (41 mg, 23% yield) as an off white solid.

LCMS m/z 378.2 (M+H)⁺ (ES⁺); 376.1 (M−H)⁻ (ES⁻).

¹H NMR (DMSO-d₆) δ 12.88 (s, 1H), 8.84 (s, 1H), 7.90 (s, 1H), 6.95 (s, 1H), 3.35 (t, J=6.1 Hz, 2H), 3.21 (s, 3H), 3.10 (app q, J=6.0 Hz, 2H), 2.83 (t, J=7.4 Hz, 4H), 2.64 (t, J=7.4 Hz, 4H), 1.98 (p, J=7.4 Hz, 4H).

### Example 84: 5-((1,2,3,5,6,7-hexahydro-s-indacen-4-yl)amino)-N-(1-methylpyrrolidin-3-yl)-4H-1,2,4-triazole-3-sulfonamide

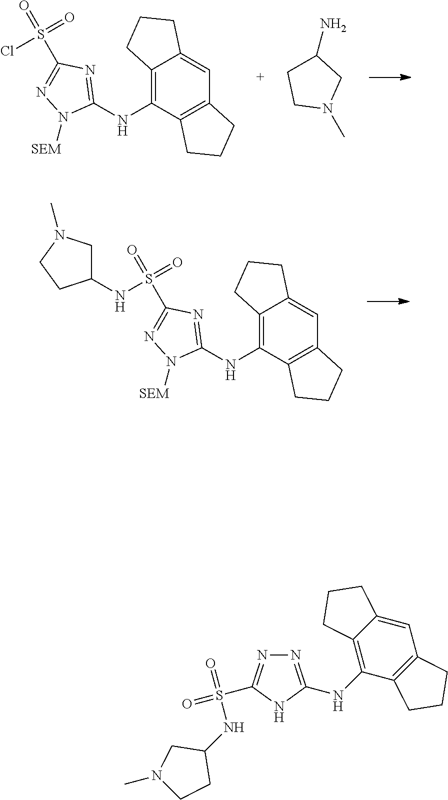

Step A: Prepared according to the general procedure of 5-((1,2,3,5,6,7-hexahydro-s-indacen-4-yl)amino)-N-(2-methoxyethyl)-1-((2-(trimethylsilyl)ethoxy)methyl)-1H-1,2,4-triazole-3-sulfonamide (Example 83, Step A) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and 1-methylpyrrolidin-3-amine to afford 5-((1,2,3,5,6,7-hexahydro-s-indacen-4-yl)amino)-N-(1-methylpyrrolidin-3-yl)-1-((2-(trimethylsilyl)ethoxy)methyl)-1H-1,2,4-triazole-3-sulfonamide (114 mg, 16% yield) as a sticky yellow oil.

LCMS m/z 533.4 (M+H)⁺ (ES⁺).

¹H NMR (DMSO-d₆) δ 8.74 (s, 1H), 8.18 (d, J=7.2 Hz, 1H), 6.98 (s, 1H), 5.50 (s, 2H), 3.76-3.65 (m, 1H), 3.62 (t, J=8.1 Hz, 2H), 2.84 (t, J=7.2 Hz, 4H), 2.66 (t, J=7.4 Hz, 4H), 2.59-2.51 (m, 1H), 2.42-2.30 (m, 2H), 2.21-2.15 (m, 1H), 2.16 (s, 3H), 1.97 (p, J=7.3 Hz, 4H), 1.95-1.87 (m, 1H), 1.62-1.51 (m, 1H), 0.94-0.86 (m, 2H), −0.01 (s, 9H).

Step B: Prepared according to the general procedure of 5-((1,2,3,5,6,7-hexahydro-s-indacen-4-yl)amino)-N-(2-methoxyethyl)-4H-1,2,4-triazole-3-sulfonamide (Example 83, Step B) from 5-((1,2,3,5,6,7-hexahydro-s-indacen-4-yl)amino)-N-(1-methylpyrrolidin-3-yl)-1-((2-(trimethylsilyl)ethoxy)methyl)-1H-1,2,4-triazole-3-sulfonamide to afford the title compound (16 mg, 24% yield) as a pale white solid.

LCMS m/z 403.0 (M+H)⁺ (ES⁺); 401.3 (M−H)⁻ (ES⁻).

¹H NMR (DMSO-d₆) δ 8.88 (s, 1H), 8.05 (d, J=7.1 Hz, 1H), 6.95 (s, 1H), 3.84-3.75 (m, 1H), 2.83 (t, J=7.4 Hz, 4H), 2.64 (t, J=7.4 Hz, 4H), 2.62-2.58 (m, 1H), 2.44-2.35 (m, 2H), 2.25-2.21 (m, 1H), 2.19 (s, 3H), 2.03-1.93 (m, 5H), 1.65-1.57 (m, 1H). One exchangeable proton not observed.

### Example 85: 5-((1,2,3,5,6,7-hexahydro-s-indacen-4-yl)amino)-N-phenyl-4H-1,2,4-triazole-3-sulfonamide

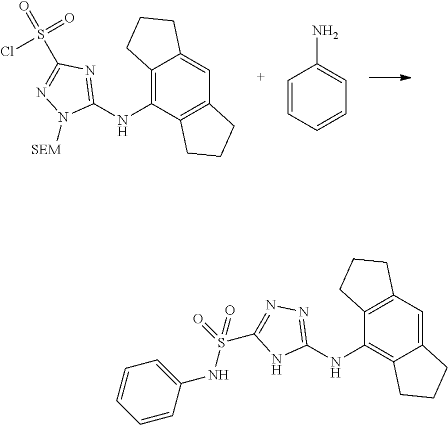 [SMILES: C[Si](C)(C)CCOCn1nc(S(=O)(=O)Cl)nc1Nc1c2c(cc3c1CCC3)CCC2 ; Nc1ccccc1 ; O=S(=O)(Nc1ccccc1)c1nnc(Nc2c3c(cc4c2CCC4)CCC3)[nH]1]

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and aniline to afford the title compound (9 mg, 3% yield) as a light white solid.

LCMS m/z 396.3 (M+H)⁺ (ES⁺).

¹H NMR (DMSO-d₆) δ 12.97 (s, 1H), 10.51 (s, 1H), 8.80 (s, 1H), 7.28-7.22 (m, 2H), 7.19-7.14 (m, 2H), 7.07-7.01 (m, 1H), 6.93 (s, 1H), 2.80 (t, J=7.4 Hz, 4H), 1.92 (p, J=7.4 Hz, 4H). Two CH₂ peaks obscured by solvent.

### Example 86: N-benzyl-5-((1,2,3,5,6,7-hexahydro-s-indacen-4-yl)amino)-4H-1,2,4-triazole-3-sulfonamide

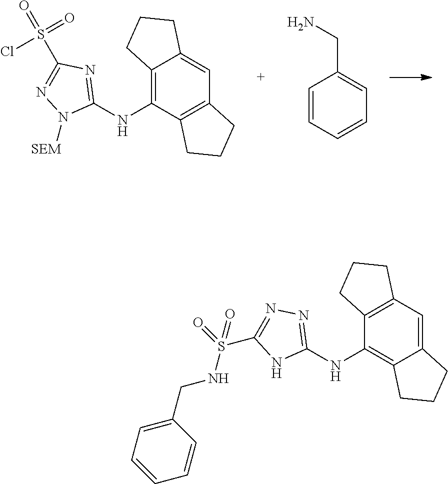 [SMILES: C[Si](C)(C)CCOCn1nc(S(=O)(=O)Cl)nc1Nc1c2c(cc3c1CCC3)CCC2 ; NCc1ccccc1 ; O=S(=O)(NCc1ccccc1)c1nnc(Nc2c3c(cc4c2CCC4)CCC3)[nH]1]

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and phenylmethanamine to afford the title compound (19 mg, 6% yield) as a pale white solid.

LCMS m/z 410.3 (M+H)⁺ (ES⁺).

¹H NMR (DMSO-d₆) δ 12.89 (s, 1H), 8.87 (s, 1H), 8.39 (d, J=6.7 Hz, 1H), 7.35-7.21 (m, 5H), 6.96 (s, 1H), 4.18 (d, J=6.1 Hz, 2H), 2.83 (t, J=7.4 Hz, 4H), 2.65 (t, J=7.3 Hz, 4H), 1.98 (P, J=7.4 Hz, 4H).

### Example 87: 5-((1,2,3,5,6,7-hexahydro-s-indacen-4-yl)amino)-N-methyl-N-(1-methylpyrrolidin-3-yl)-4H-1,2,4-triazole-3-sulfonamide

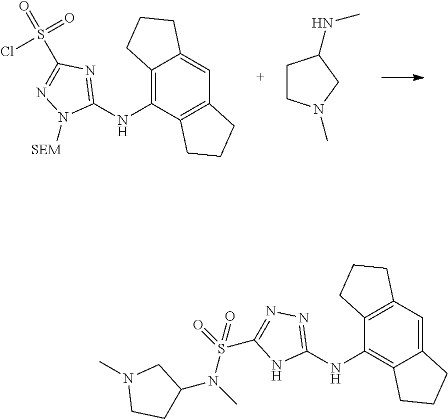

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and N,1-dimethylpyrrolidin-3-amine to afford the title compound (8 mg, 3% yield) as a pale white solid.

LCMS m/z 417.0 (M+H)⁺ (ES⁺).

¹H NMR (DMSO-d₆) δ 8.96 (s, 1H), 6.96 (s, 1H), 4.46-4.38 (m, 1H), 2.85-2.83 (m, 4H), 2.82 (s, 3H), 2.70-2.66 (m, 1H), 2.64 (t, J=7.2 Hz, 4H), 2.49-2.45 (m, 1H), 2.29-2.23 (m, 1H), 2.19 (s, 3H), 2.17-2.11 (m, 1H), 1.98 (p, J=7.4 Hz, 4H), 1.94-1.87 (m, 1H), 1.74-1.63 (m, 1H). One exchangeable proton not observed.

### Example 88: 5-((1,2,3,5,6,7-hexahydro-s-indacen-4-yl)amino)-N-((1-meth azetidin-3-yl)methyl)-4H-1,2,4-triazole-3-sulfonamide

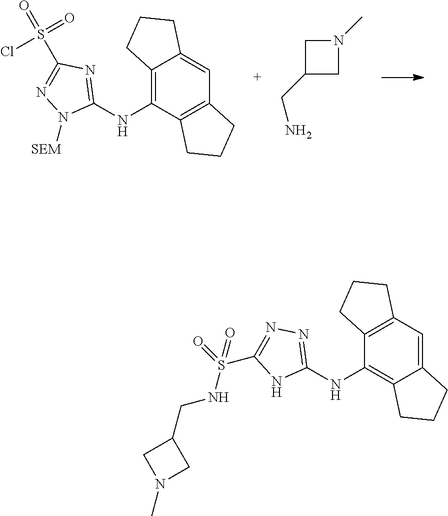

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and (1-methylazetidin-3-yl)methanamine to afford the title compound (24 mg, 13% yield) as a flocculent white solid.

LCMS m/z 403.2 (M+H)⁺ (ES⁺).

¹H NMR (DMSO-d₆) δ 9.18 (s, 1H), 8.30 (s, 1H), 6.93 (s, 1H), 3.51 (t, J=8.4 Hz, 2H), 3.28-3.21 (m, 2H), 3.13 (d, J=7.0 Hz, 2H), 2.82 (t, J=7.4 Hz, 4H), 2.65 (t, J=7.3 Hz, 4H), 2.62-2.56 (m, 1H), 2.41 (s, 3H), 1.97 (p, J=7.4 Hz, 4H). One exchangeable proton not observed.

### Example 89:N-(2-(azetidin-1-yl)ethyl)-5-((1,2,3,5,6,7-hexahydro-s-indacen-4-yl)amino)-4H-1,2,4-triazole-3-sulfonamide

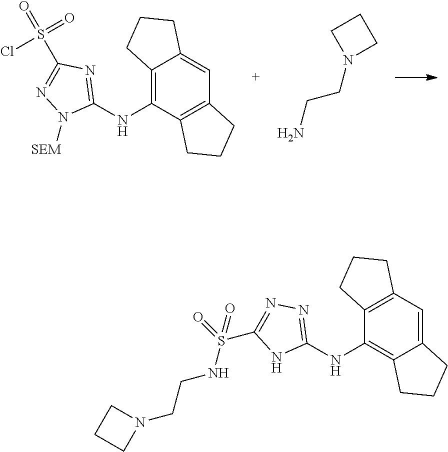 [SMILES: C[Si](C)(C)CCOCn1nc(S(=O)(=O)Cl)nc1Nc1c2c(cc3c1CCC3)CCC2 ; NCCN1CCC1 ; O=S(=O)(NCCN1CCC1)c1nnc(Nc2c3c(cc4c2CCC4)CCC3)[nH]1]

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and 2-(azetidin-1-yl)-ethanamine to afford the title compound (41 mg, 24% yield) as a flocculent white solid.

LCMS m/z 403.3 (M+H)⁺ (ES⁺); 401.1 (M−H)⁻ (ES⁻).

¹H NMR (DMSO-d₆) δ 9.03 (s, 1H), 8.23 (s, 1H), 6.95 (s, 1H), 3.41 (t, J=7.4 Hz, 4H), 2.96 (t, J=6.7 Hz, 2H), 2.83 (t, J=7.4 Hz, 4H), 2.71-2.61 (m, 6H), 2.06 (p, J=7.4 Hz, 2H), 1.98 (p, J=7.4 Hz, 4H). One exchangeable proton not observed.

### Example 90: 5-((1,2,3,5,6,7-hexahydro-s-indacen-4-yl)amino)-N-methyl-N-(1-methylpiperidin-4-yl)-4H-1,2,4-triazole-3-sulfonamide

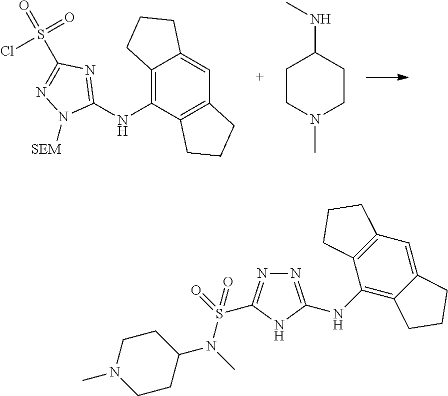

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and N,1-dimethylpiperidin-4-amine to afford the title compound (45 mg, 24% yield) as a flocculent white solid.

LCMS m/z 431.0 (M+H)⁺ (ES⁺); 429.3 (M−H)⁻ (ES⁻).

¹H NMR (DMSO-d₆) δ 9.01 (s, 1H), 6.96 (s, 1H), 3.79-3.70 (m, 1H), 3.02-2.94 (m, 2H), 2.83 (t, J=7.4 Hz, 4H), 2.76 (s, 3H), 2.63 (t, J=7.3 Hz, 4H), 2.33 (s, 3H), 2.32-2.25 (m, 2H), 1.97 (p, J=7.4 Hz, 4H), 1.86-1.75 (m, 2H), 1.56-1.49 (m, 2H). One exchangeable proton not observed.

### Example 91: 5-((3-(dimethylamino)azetidin-1-yl)sulfonyl)-N-(1,2,3,5,6,7-hexahydro-s-indacen-4-yl)-4H-1,2,4-triazol-3-amine

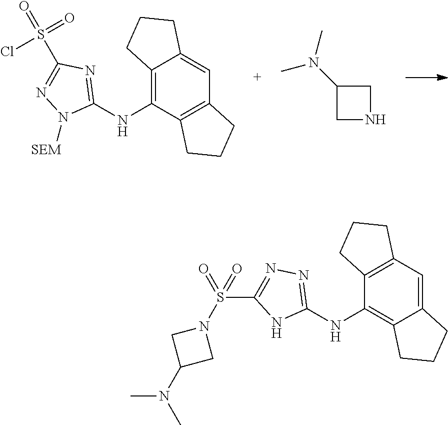

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and N,N-dimethylazetidin-3-amine to afford the title compound (44 mg, 25% yield) as a flocculent white solid.

LCMS m/z 403.5 (M+H)⁺ (ES⁺); 401.3 (M−H)⁻ (ES⁻).

¹H NMR (DMSO-d₆) δ 8.99 (s, 1H), 6.98 (s, 1H), 3.91-3.83 (m, 2H), 3.78-3.72 (m, 2H), 3.04 (p, J=6.8 Hz, 1H), 2.84 (t, J=7.4 Hz, 4H), 2.66 (t, J=7.3 Hz, 4H), 2.03-1.92 (m, 10H). One exchangeable proton not observed.

### Example 92:5-((1,2,3,5,6,7-hexahydro-s-indacen-4-yl)amino)-N-methyl-N-((1-methylpyrrolidin-3-yl)methyl)-4H-1,2,4-triazole-3-sulfonamide

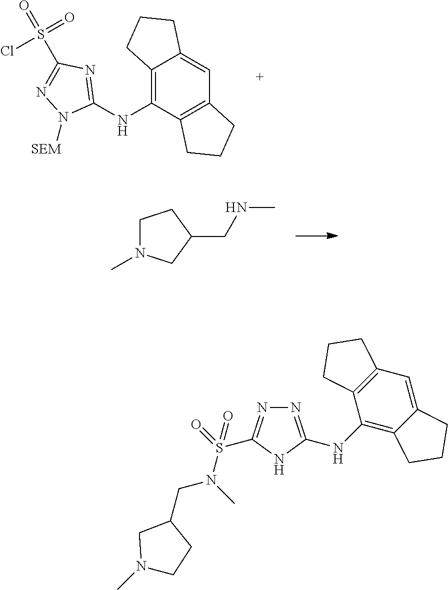

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and N-methyl-1-(1-methyl-pyrrolidin-3-yl)methanamine to afford the title compound (33 mg, 18% yield) as a flocculent white solid.

LCMS m/z 431.4 (M+H)⁺ (ES⁺); 429.3 (M−H)⁻ (ES⁻).

¹H NMR (DMSO-d₆) δ 9.20 (s, 1H), 8.27 (s, 1H), 6.95 (s, 1H), 3.08 (d, J=7.3 Hz, 2H), 2.85-2.75 (m, 9H), 2.72-2.61 (m, 5H), 2.43 (s, 3H), 2.02-1.89 (m, 6H), 1.58-1.47 (m, 1H). One exchangeable proton not observed.

### Example 93: 5-((3-(dimethylamino)pyrrolidin-1-yl)sulfonyl)-N-(1,2,3,5,6,7-hexahydro-s-indacen-4-yl)-4H-1,2,4-triazol-3-amine

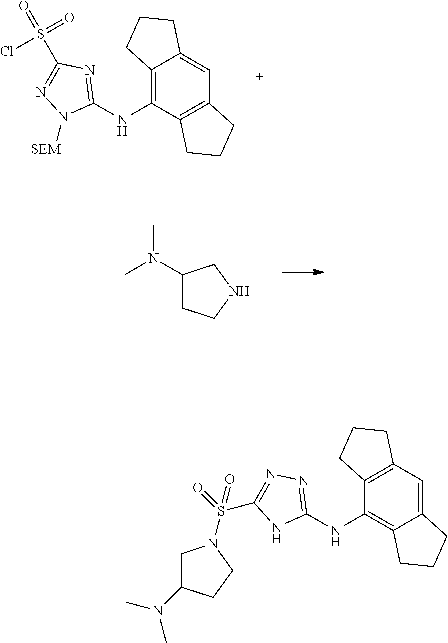

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and N,N-dimethylpyrrolidin-3-amine to afford the title compound (48 mg, 26% yield) as a flocculent white solid. LCMS m/z 417.3 (M+H)⁺ (ES⁺); 415.5 (M−H)⁻ (ES⁻).

¹H NMR (DMSO-d₆) δ 8.95 (s, 1H) 6.96 (s, 1H), 3.58-3.50 (m, 1H), 3.50-3.43 (m, 1H), 3.34-3.26 (m, 1H), 3.15-3.09 (m, 1H), 2.82 (t, J=7.5 Hz, 4H), 2.64 (t, J=7.3 Hz, 4H), 2.20 (s, 6H), 2.07-1.91 (m, 6H), 1.77-1.66 (m, 1H). One exchangeable proton not observed.

### Example 94: N-(2-(dimethylamino)ethyl)-5-((1,2,3,5,6,7-hexahydro-s-indacen-4-yl)amino)-4H-1,2,4-triazole-3-sulfonamide

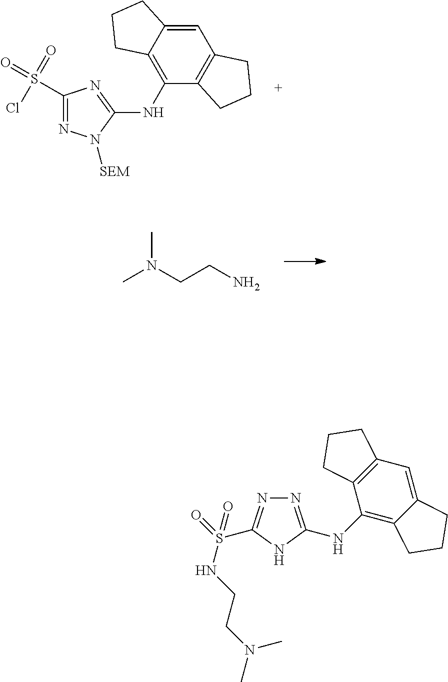

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and N¹,N¹-dimethylethane-1,2-diamine to afford the title compound (13 mg, 9% yield) as a flocculent white solid.

LCMS m/z 391.1 (M+H)⁺ (ES⁺); 389.2 (M−H)⁻ (ES⁻).

¹H NMR (DMSO-d₆) δ 8.93 (s, 1H), 8.18 (s, 1H), 6.94 (s, 1H), 3.05 (t, J=7.0 Hz, 2H), 2.82 (t, J=7.4 Hz, 4H), 2.64 (t, J=7.3 Hz, 4H), 2.40 (t, J=7.0 Hz, 2H), 2.18 (s, 6H), 1.97 (p, J=7.4 Hz, 4H). One exchangeable proton not observed.

### Example 95: 5-((1,2,3,5,6,7-hexahydro-s-indacen-4-yl)amino)-N-((1-methylpyrrolidin-2-yl)methyl)-4H-1,2,4-triazole-3-sulfonamide

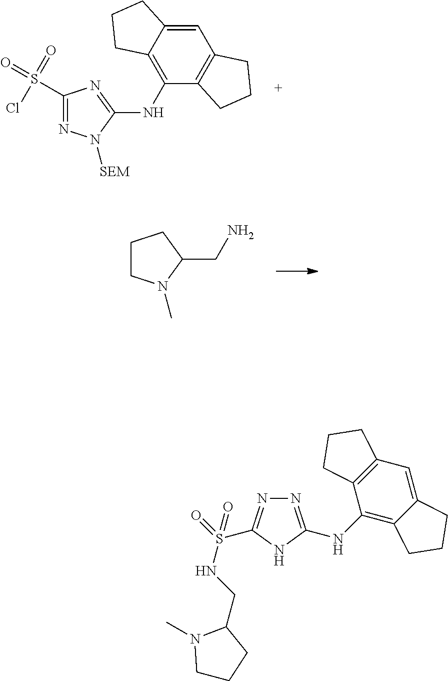

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and (1-methylpyrrolidin-2-yl)methanamine to afford the title compound (9 mg, 6% yield) as a flocculent white solid.

LCMS m/z 417.5 (M+H)⁺ (ES⁺).

¹H NMR (DMSO-d₆) δ 8.89 (s, 1H), 8.18 (s, 1H), 6.95 (s, 1H), 3.15-3.09 (m, 1H), 3.00-2.94 (m, 1H), 2.87-2.79 (m, 5H), 2.65 (t, J=7.4 Hz, 4H), 2.40-2.32 (m, 1H), 2.28 (s, 3H), 2.26-2.18 (m, 1H), 1.97 (p, J=7.4 Hz, 4H), 1.90-1.81 (m, 1H), 1.68-1.57 (m, 2H), 1.57-1.47 (m, 1H). One exchangeable proton not observed.

### Example 96: 5-((1,2,3,5,6,7-hexahydro-s-indacen-4-yl)amino)-N-(pyridin-4-ylmethyl)-4H-1,2,4-triazole-3-sulfonamide

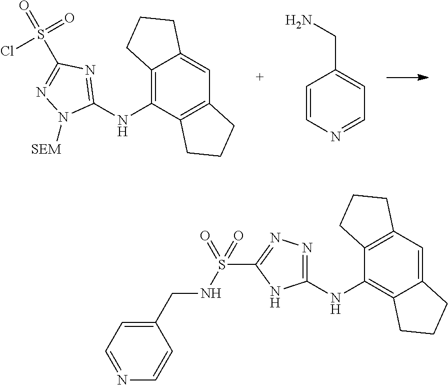 [SMILES: C[Si](C)(C)CCOCn1nc(S(=O)(=O)Cl)nc1Nc1c2c(cc3c1CCC3)CCC2 ; NCc1ccncc1 ; O=S(=O)(NCc1ccncc1)c1nnc(Nc2c3c(cc4c2CCC4)CCC3)[nH]1]

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and pyridin-4-yl-methanamine to afford the title compound (12 mg, 7% yield) as a pale yellow solid.

LCMS m/z 411.3 (M+H)⁺ (ES⁺); 409.2 (M−H)⁻ (ES⁻).

¹H NMR (DMSO-d₆) δ 13.00 (s, 1H), 8.91 (s, 1H), 8.56 (t, J=6.3 Hz, 1H), 8.52-8.44 (m, 2H), 7.34-7.24 (m, 2H), 6.95 (s, 1H), 4.22 (d, J=5.4 Hz, 2H), 2.82 (t, J=7.4 Hz, 4H), 2.64 (t, J=7.3 Hz, 4H), 1.97 (p, J=7.2 Hz, 4H).

### Example 97: 5-((1,2,3,5,6,7-hexahydro-s-indacen-4-yl)amino)-N-(pyridin-3-ylmethyl)-4H-1,2,4-triazole-3-sulfonamide

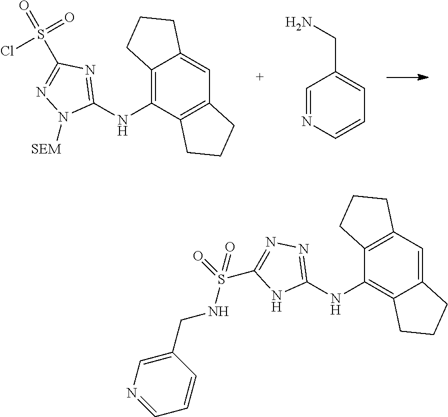 [SMILES: C[Si](C)(C)CCOCn1nc(S(=O)(=O)Cl)nc1Nc1c2c(cc3c1CCC3)CCC2 ; NCc1cccnc1 ; O=S(=O)(NCc1cccnc1)c1nnc(Nc2c3c(cc4c2CCC4)CCC3)[nH]1]

5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) (167 mg, 0.356 mmol) in DCM (6.5 mL) was added to a solution of pyridin-3-ylmethanamine (0.073 mL, 0.712 mmol), triethylamine (0.119 mL, 0.853 mmol) and DCM (1 mL) and the reaction was stirred for 1 h. The reaction was concentrated in vacuo, HCl (4 N in dioxane,5 mL, 20.00 mmol) was added and stirred at RT overnight. An additional 1 mL of 4M HCl in dioxane was added and stirred over the weekend. TFA (0.5 mL) was added to the reaction mixture and stirred for 1 h at RT. The reaction was concentrated in vacuo and the crude product was purified by acidic prep HPLC (35-65% MeOH in water) to afford the title compound (11 mg, 7% yield) as a flocculent white solid.

LCMS m/z 411.2 (M+H)⁺ (ES⁺); 409.2 (M−H)⁻ (ES⁻).

¹H NMR (DMSO-d₆) δ 12.96 (s, 1H), 8.88 (s, 1H), 8.52-8.44 (m, 3H), 7.72-7.66 (m, 1H), 7.36-7.31 (m, 1H), 6.96 (s, 1H), 4.22 (d, J=5.7 Hz, 2H), 2.83 (t, J=7.4 Hz, 4H), 2.64 (t, J=7.4 Hz, 4H), 1.98 (p, J=7.4 Hz, 4H).

### Example 98: 5-((1,2,3,5,6,7-hexahydro-s-indacen-4-yl)amino)-N-(pyridin-2-ylmethyl)-4H-1,2,4-triazole-3-sulfonamide

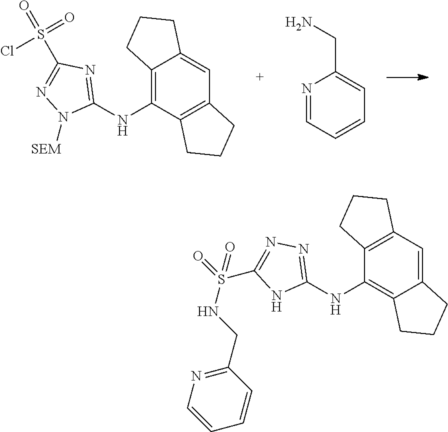 [SMILES: C[Si](C)(C)CCOCn1nc(S(=O)(=O)Cl)nc1Nc1c2c(cc3c1CCC3)CCC2 ; NCc1ccccn1 ; O=S(=O)(NCc1ccccn1)c1nnc(Nc2c3c(cc4c2CCC4)CCC3)[nH]1]

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and pyridin-2-yl-methanamine to afford the title compound (18 mg, 19% yield) as a light white solid.

LCMS m/z 411.3 (M+H)⁺ (ES⁺).

¹H NMR (DMSO-d₆) δ 12.96 (s, 1H), 8.89 (s, 1H), 8.52-8.44 (m, 2H), 7.81-7.73 (m, 1H), 7.41 (d, J=7.8 Hz, 1H), 7.30-7.25 (m, 1H), 6.95 (s, 1H), 4.27 (s, 2H), 2.83 (t, J=7.4 Hz, 4H), 2.64 (t, J=7.3 Hz, 4H), 1.98 (p, J=7.4 Hz, 4H).

### Example 99: 5-((1,2,3,5,6,7-hexahydro-s-indacen-4-yl)amino)-N-(1-methyl-1H-pyrazol-4-yl)-4H-1,2,4-triazole-3-sulfonamide

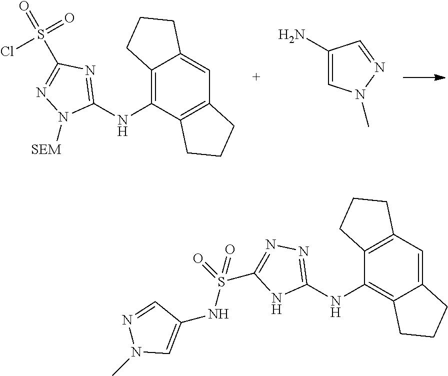

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and 1-methyl-1H-pyrazol-4-amine to afford the title compound (21 mg, 24% yield) as a pale brown solid.

LCMS m/z 400.3 (M+H)⁺ (ES⁺).

¹H NMR (DMSO-d₆) δ 12.93 (s, 1H), 9.91 (s, 1H), 8.80 (s, 1H), 7.48 (s, 1H), 7.11 (s, 1H), 6.95 (s, 1H), 3.74 (s, 3H), 2.82 (t, J=7.4 Hz, 4H), 2.60 (t, J=7.3 Hz, 4H), 1.97 (p, J=7.4 Hz, 4H).

### Example 100: N-(1,2,3,5,6,7-hexahydro-s-indacen-4-yl)-5-((4-methylpiperazin-1-yl)sulfonyl)-4H-1,2,4-triazol-3-amine

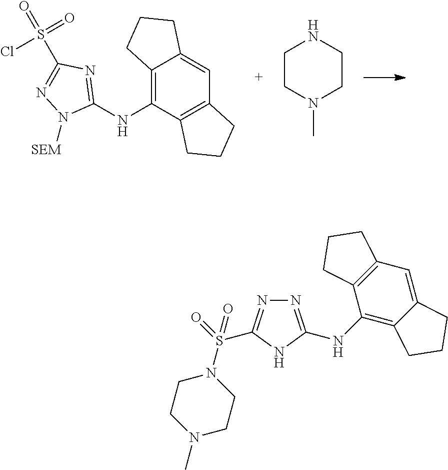

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and 1-methylpiperazine to afford the title compound (20 mg, 23% yield) as a flocculent white solid.

LCMS m/z 403.3 (M+H)⁺ (ES⁺).

¹H NMR (DMSO-d₆) δ 13.23 (s, 1H), 9.04 (s, 1H), 6.98 (s, 1H), 3.00 (br s, 4H), 2.84 (t, J=7.4 Hz, 4H), 2.64 (t, J=7.3 Hz, 4H), 2.60 (br s, 4H), 1.99 (p, J=7.4 Hz, 4H). Three protons obscured by solvent.

### Example 101: N-(1,2,3,5,6,7-hexahydro-s-indacen-4-yl)-5-((1-methylhexahydropyrrolo[3,4-b]pyrrol-5 (1H)-yl)sulfonyl)-4H-1,2,4-triazol-3-amine

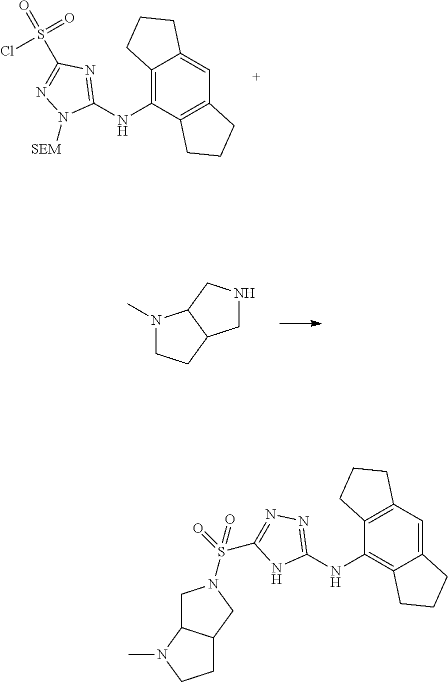

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and 1-methyl-octahydropyrrolo[3,4-b]pyrrole to afford the title compound (20 mg, 21% yield) as a flocculent white solid.

LCMS m/z 429.34 (M+H)⁺ (ES⁺).

¹H NMR (DMSO-d₆) δ 8.95 (s, 1H), 6.96 (s, 1H), 3.47-3.41 (m, 1H), 3.36-3.30 (m, 1H), 3.26-3.21 (m, 1H), 3.17-3.11 (m, 2H), 3.09-3.03 (m, 1H), 2.86-2.76 (m, 5H), 2.64 (t, J=7.3 Hz, 4H), 2.47 (t, J=8.8 Hz, 1H), 2.38 (s, 3H), 2.09-2.02 (m, 1H), 1.98 (p, J=7.5 Hz, 4H), 1.61-1.51 (m, 1H). One exchangeable proton not observed.

### Example 102:5-((1,2,3,5,6,7-hexahydro-s-indacen-4-yl)amino)-N-(1-methylpiperidin-4-yl)-4H-1,2,4-triazole-3-sulfonamide

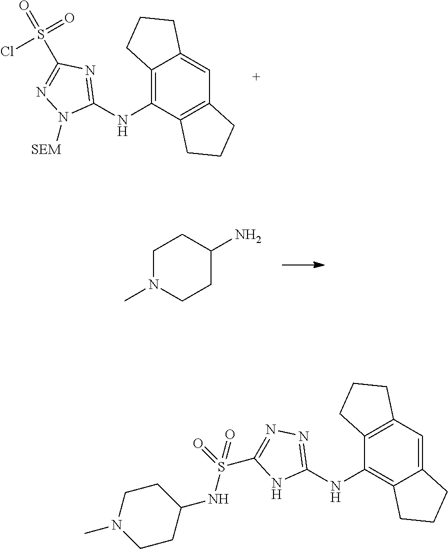

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and 1-methylpiperidin-4-amine to afford the title compound (15 mg, 16% yield) as a white solid.

LCMS m/z 417.35 (M+H)⁺ (ES⁺).

¹H NMR (DMSO-d₆): δ 8.86 (s, 1H), 7.86 (d, J=7.1 Hz, 1H), 6.94 (s, 1H), 3.11-3.09 (m, 1H), 2.82 (t, J=7.4 Hz, 4H), 2.71-2.68 (m, 2H), 2.63 (t, J=7.2 Hz, 4H), 2.15 (s, 3H), 1.99-1.90 (m, 6H), 1.69-1.65 (m, 2H), 1.49-1.41 (m, 2H). One exchangeable proton not observed.

### Example 103: 5-((1,2,3,5,6,7-hexahydro-s-indacen-4-yl)amino)-N-((6-oxo-1,6-dihydropyridin-2-yl)methyl)-4H-1,2,4-triazole-3-sulfonamide

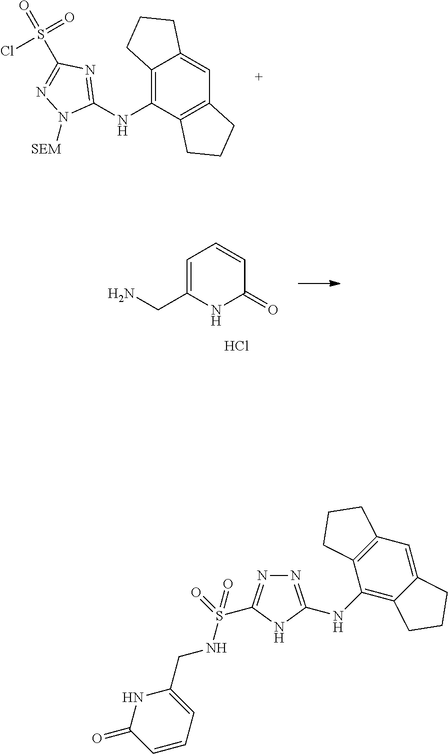 [SMILES: C[Si](C)(C)CCOCn1nc(S(=O)(=O)Cl)nc1Nc1c2c(cc3c1CCC3)CCC2 ; NCc1cccc(=O)[nH]1 ; Cl ; O=c1cccc(CNS(=O)(=O)c2nnc(Nc3c4c(cc5c3CCC5)CCC4)[nH]2)[nH]1]

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and 6-(aminomethyl)-pyridin-2 (1H)-one hydrochloride to afford the title compound (4 mg, 4% yield) as a yellow solid.

LCMS m/z 427.2 (M+H)⁺ (ES⁺).

¹H NMR (DMSO-d₆). δ 12.93 (s, 1H), 8.88 (br s, 1H), 8.48 (t, J=6.3 Hz, 1H), 7.36 (dd, J=9.1, 6.8 Hz, 1H), 6.96 (s, 1H), 6.20 (d, J=9.1 Hz, 1H), 6.12 (br s, 1H), 4.04 (d, J=6.2 Hz, 2H), 2.82 (t, J=7.5 Hz, 4H), 2.63 (t, J=7.3 Hz, 4H), 1.97 (p, J=7.4 Hz, 4H). One exchangeable proton not observed.

### Example 104: 5-([1,3′-bipyrrolidin]-1′-ylsulfonyl)-N-(1,2,3,5,6,7-hexahydro-s-indacen-4-yl)-4H-1,2,4-triazol-3-amine

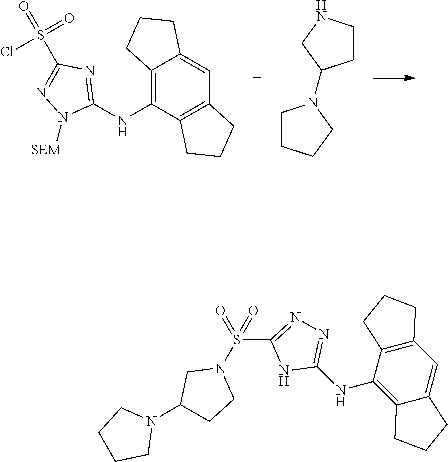 [SMILES: C[Si](C)(C)CCOCn1nc(S(=O)(=O)Cl)nc1Nc1c2c(cc3c1CCC3)CCC2 ; C1CCN(C2CCNC2)C1 ; O=S(=O)(c1nnc(Nc2c3c(cc4c2CCC4)CCC3)[nH]1)N1CCC(N2CCCC2)C1]

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 1,3′-bipyrrolidine and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B⁴) to afford the title compound (29 mg, 29% yield) as a white solid.

LCMS m/z 443.3 (M+H)⁺ (ES⁺).

¹H NMR (DMSO-d₆): δ 8.93 (s, 1H), 6.96 (s, 1H), 3.51-3.41 (m, 2H), 3.34-3.29 (m, 4H), 3.10 (dd, J=9.9, 7.7 Hz, 1H), 2.83 (t, J=7.4 Hz, 4H), 2.64 (t, J=7.4 Hz, 4H), 2.41-2.31 (m, 4H), 2.09-1.89 (m, 4H), 1.65 (m, 4H). One exchangeable proton not observed. Multiplet at 3.34-3.29 ppm obscured by water peak.

### Example 105: (S)-5-((1,2,3,5,6,7-hexahydro-s-indacen-4-yl)amino)-N-(1-methylpiperidin-3-yl)-4H-1,2,4-triazole-3-sulfonamide

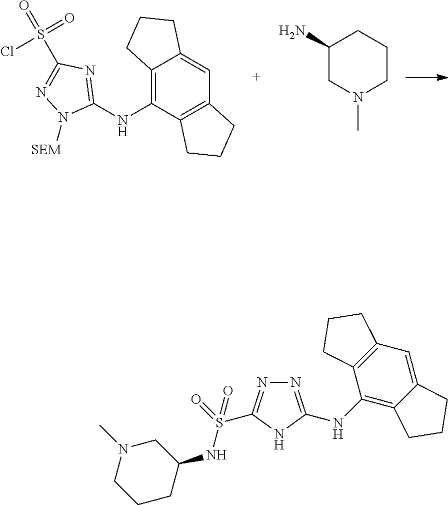

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from (S)-1-methylpiperidin-3-amine dihydrochloride and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) to afford the title compound (11 mg, 12% yield) as a white solid.

LCMS m/z 417.3 (M+H)⁺ (ES⁺).

¹H NMR (DMSO-d₆): δ 8.82 (s, 1H), 7.88 (d, J=7.5 Hz, 1H), 6.95 (s, 1H), 3.23-3.21 (m, 1H), 2.83 (t, J=7.4 Hz, 4H), 2.72-2.68 (m, 1H), 2.64 (t, J=7.4 Hz, 4H), 2.61-2.55 (m, 1H), 2.12 (s, 3H), 1.97 (p, J=7.4 Hz, 4H), 1.83-1.66 (m, 3H), 1.65-1.54 (m, 1H), 1.43-1.30 (m, 1H), 1.19-1.03 (m, 1H). One exchangeable proton not observed.

### Example 106: (S)—N-(1-(dimethylamino)propan-2-yl)-5-((1,2,3,5,6,7-hexahydro-s-indacen-4-yl)amino)-4H-1,2,4-triazole-3-sulfonamide

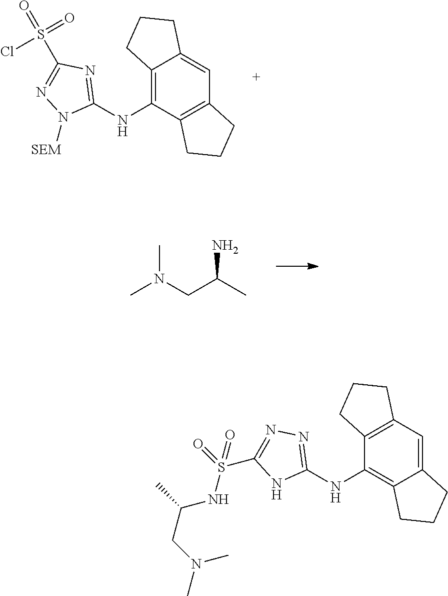

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from (S)—N¹,N¹-dimethylpropane-1,2-diamine and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) to afford the title compound (7 mg, 7% yield) as an off white solid.

LCMS m/z 405.0 (M+H)⁺ (ES⁺).

¹H NMR (DMSO-d₆) δ 8.82 (s, 1H), 7.62 (s, 1H), 6.95 (s, 1H), 3.43-3.37 (m, 1H), 2.83 (t, J=7.4 Hz, 4H), 2.65 (t, J=7.4 Hz, 4H), 2.18 (d, J=7.2 Hz, 2H), 2.11 (s, 6H), 1.97 (p, J=7.4 Hz, 4H), 1.02 (d, J=6.5 Hz, 3H). One exchangeable proton not observed.

### Example 107:N-(2-(5-((1,2,3,5,6,7-hexahydro-s-indacen-4-yl)amino)-N-methyl-4H-1,2,4-triazole-3-sulfonamido)ethyl)acetamide

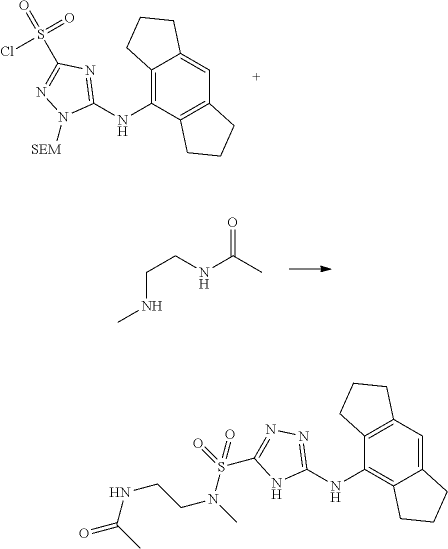

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from N-(2-(methylamino)ethyl)acetamide, HCl and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) to afford the title compound (16 mg, 17% yield) as an off white solid.

LCMS m/z 419.1 (M+H)⁺ (ES⁺).

¹H NMR (DMSO-d₆) δ 13.01 (s, 1H), 8.93 (s, 1H), 7.96 (t, J=5.7 Hz, 1H), 6.96 (s, 1H), 3.21 (t, J=6.1 Hz, 2H), 3.17-3.10 (m, 2H), 2.88-2.77 (m, 7H), 2.63 (t, J=7.3 Hz, 4H), 1.98 (p, J=7.4 Hz, 4H), 1.79 (s, 3H).

### Example 108: 5-((1,2,3,5,6,7-hexahydro-s-indacen-4-yl)amino)-N-(2-(pyrrolidin-1-yl)ethyl)-4H-1,2,4-triazole-3-sulfonamide

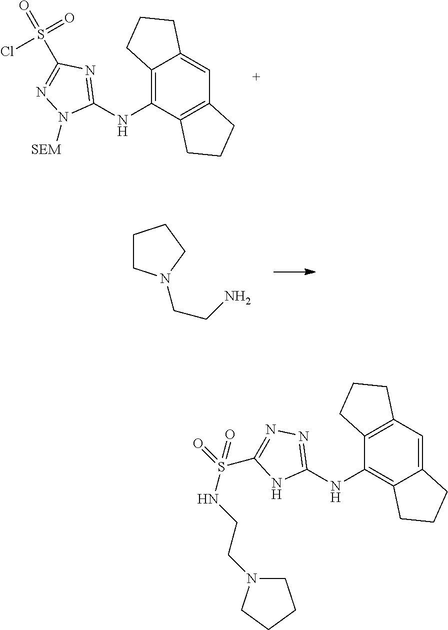 [SMILES: C[Si](C)(C)CCOCn1nc(S(=O)(=O)Cl)nc1Nc1c2c(cc3c1CCC3)CCC2 ; NCCN1CCCC1 ; O=S(=O)(NCCN1CCCC1)c1nnc(Nc2c3c(cc4c2CCC4)CCC3)[nH]1]

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 2-(pyrrolidin-1-yl)ethanamine and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) to afford the title compound (16 mg, 17% yield) as an off white solid.

LCMS m/z 417.3 (M+H)⁺ (ES⁺).

¹H NMR (DMSO-d₆) δ 8.88 (s, 1H), 7.97 (s, 1H), 6.96 (s, 1H), 3.17 (t, J=6.8 Hz, 2H), 2.92-2.72 (m, 10H), 2.65 (t, J=7.3 Hz, 4H), 1.98 (p, J=7.4 Hz, 4H), 1.81-1.71 (m, 4H). One exchangeable proton not observed.

### Example 109: N-(3-(diethylamino)propyl)-5-((1,2,3,5,6,7-hexahydro-s-indacen-4-yl)amino)-4H-1,2,4-triazole-3-sulfonamide

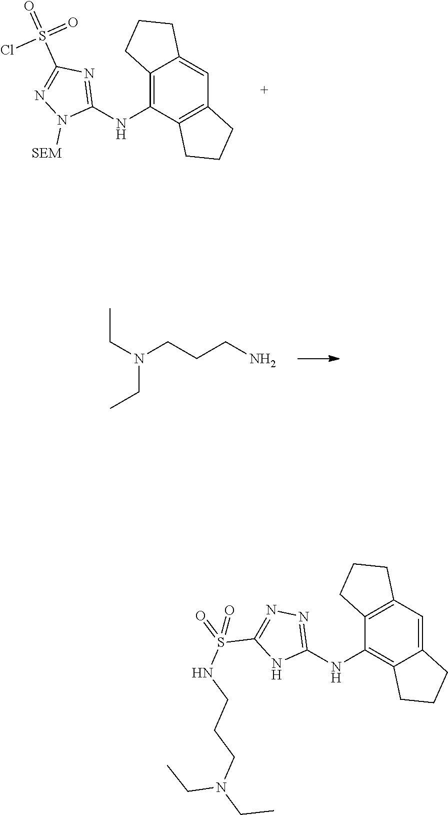

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from N¹,N¹-diethylpropane-1,3-diamine and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) to afford the title compound (22 mg, 22% yield) as an off white solid.

LCMS m/z 433.4 (M+H)⁺ (ES⁺).

¹H NMR (DMSO-d₆) δ 8.92 (s, 1H), 6.95 (s, 1H), 3.00 (t, J=6.8 Hz, 2H), 2.83 (t, J=7.4 Hz, 4H), 2.76-2.58 (m, 10H), 1.98 (p, J=7.4 Hz, 4H), 1.65 (p, J=7.0 Hz, 2H), 1.03 (t, J=7.2 Hz, 6H). Two exchangeable protons not observed.

### Example 110:N-(2-(diisopropylamino)ethyl)-5-((1,2,3,5,6,7-hexahydro-s-indacen-4-yl)amino)-4H-1,2,4-triazole-3-sulfonamide

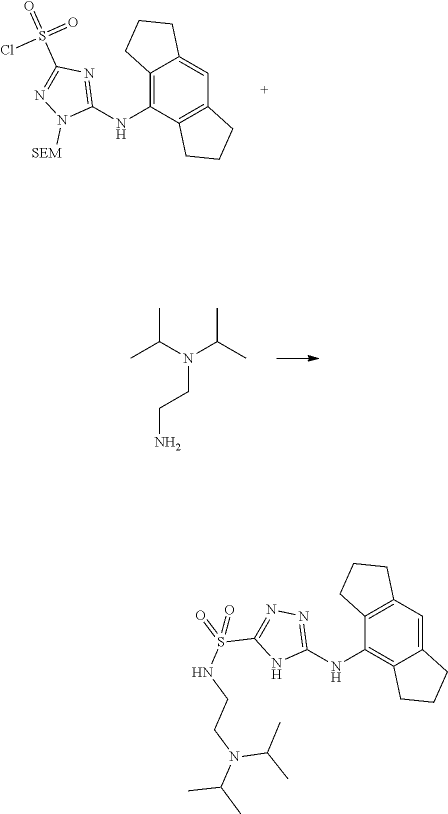

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from N¹,N¹-diisopropylethane-1,2-diamine and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) to afford the title compound (26 mg, 26% yield) as an off white solid.

LCMS m/z 447.4 (M+H)⁺ (ES⁺).

¹H NMR (DMSO-d₆) δ 12.95 (br s, 1H), 8.85 (s, 1H), 7.82 (s, 1H), 6.96 (s, 1H), 3.40-3.31 (m, 4H), 3.16-2.93 (m, 2H), 2.83 (t, J=7.4 Hz, 4H), 2.64 (t, J=7.4 Hz, 4H), 1.98 (p, J=7.4 Hz, 4H), 1.00 (s, 12H).

### Example 111: methyl 2-(N-(2-aminoethyl)-5-((1,2,3,5,6,7-hexahydro-s-indacen-4-yl)amino)-4H-1,2,4-triazole-3-sulfonamido)acetate

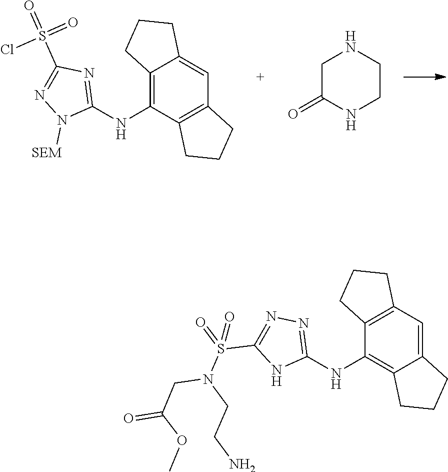 [SMILES: C[Si](C)(C)CCOCn1nc(S(=O)(=O)Cl)nc1Nc1c2c(cc3c1CCC3)CCC2 ; O=C1CNCCN1 ; COC(=O)CN(CCN)S(=O)(=O)c1nnc(Nc2c3c(cc4c2CCC4)CCC3)[nH]1]

To a stirred solution of piperazin-2-one (32.0 mg, 0.320 mmol) and Et₃N (0.045 mL, 0.320 mmol) in DCM (1 mL) was added the crude reaction mixture from the preparation of 5-((1,2,3,5,6,7-hexahydro-s-indacen-4-yl)amino)-1-((2-(trimethylsilyl)-ethoxy)methyl)-1H-1,2,4-triazole-3-sulfonyl chloride (Intermediate B4) (0.213 mmol). The reaction mixture was allowed to stir at RT for 1 h. The reaction was concentrated in vacuo. HCl (4 M in dioxane, 3 mL) was added and the reaction was stirred at RT overnight. The crude product was purified by acidic prep HPLC (35-65% MeOH in water) during which opening of the piperazin-2-one ring occurred to afford the title compound (10 mg, 11% yield) as an off white solid.

LCMS m/z 435.5 (M+H)⁺ (ES⁺).

¹H NMR (DMSO-d₆) δ 13.08 (s, 1H), 9.02 (s, 1H), 7.94 (s, 2H), 6.98 (s, 1H), 4.18 (s, 2H), 3.64 (s, 3H), 3.57 (t, J=6.7 Hz, 2H), 3.06-3.02 (m, 2H), 2.84 (t, J=7.4 Hz, 4H), 2.65 (t, J=7.4 Hz, 4H), 1.99 (p, J=7.4 Hz, 4H).

### Example 112: 5-((1,2,3,5,6,7-hexahydro-s-indacen-4-yl)amino)-N-(piperidin-3-yl)-4H-1,2,4-triazole-3-sulfonamide

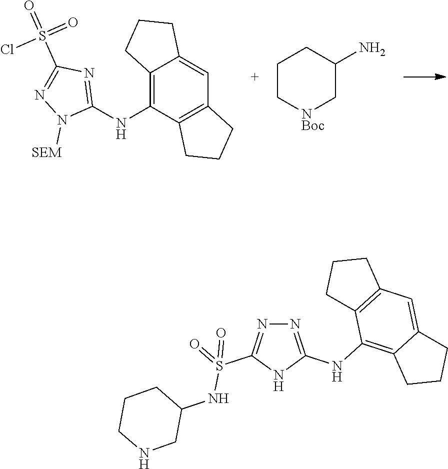 [SMILES: C[Si](C)(C)CCOCn1nc(S(=O)(=O)Cl)nc1Nc1c2c(cc3c1CCC3)CCC2 ; CC(C)(C)OC(=O)N1CCCC(N)C1 ; O=S(=O)(NC1CCCNC1)c1nnc(Nc2c3c(cc4c2CCC4)CCC3)[nH]1]

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from tert-butyl 3-aminopiperidine-1-carboxylate and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) to afford the title compound (15 mg, 17% yield) as a flocculent white powder.

LCMS m/z 403.3 (M+H)⁺ (ES⁺).

¹H NMR (DMSO-d₆) δ 9.04 (s, 1H), 6.93 (s, 1H), 3.23 (br s, 2H), 2.99-2.96 (m, 1H), 2.83-2.80 (m, 5H), 2.64 (t, J=7.3 Hz, 4H), 2.43-2.36 (m, 3H), 1.97 (p, J=7.5 Hz, 4H), 1.77-1.75 (m, 1H), 1.63-1.61 (m, 1H), 1.34-1.32 (m, 2H). One exchangeable proton not observed.

### Example 113: 4-((5-((1,2,3,5,6,7-hexahydro-s-indacen-4-yl)amino)-4H-1,2,4-triazol-3-yl)sulfonyl)-1-methylpiperazin-2-one

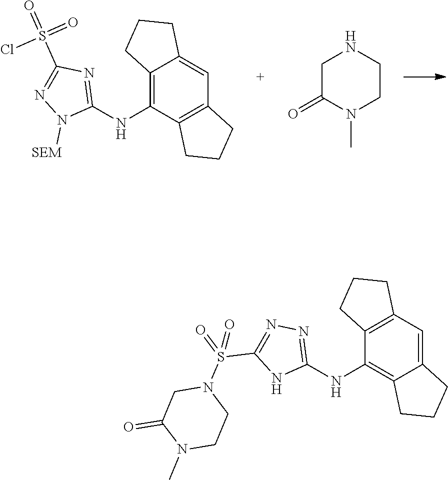

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 1-methylpiperazin-2-one and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B⁴) to afford the title compound (14 mg, 15% yield) as a white powder.

LCMS m/z 417.4 (M+H)⁺ (ES⁺).

¹H NMR (DMSO-d₆): δ 13-13 (s, 1H), 9.02 (s, 1H), 6.98 (s, 1H), 3.81 (s, 2H), 3.49-3.47 (m, 2H), 3.35-3.33 (m, 2H), 2.84-2.81 (m, 7H), 2.63 (t, J=7.4 Hz, 4H), 1.98 (p, J=7.4 Hz, 4H).

### Example 114: 5-((1,2,3,5,6,7-hexahydro-s-indacen-4-yl)amino)-N-(2-(2-oxopyrrolidin-1-yl)ethyl)-4H-1,2,4-triazole-3-sulfonamide

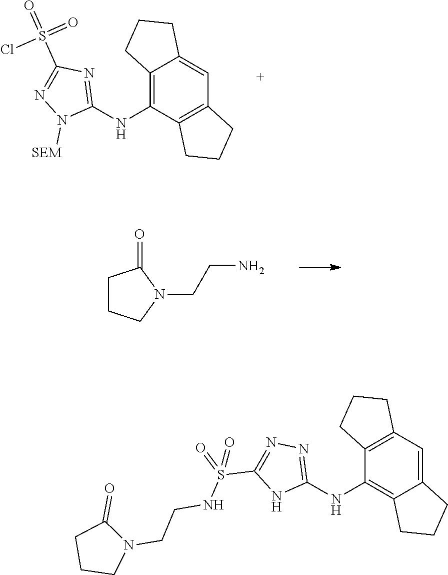 [SMILES: C[Si](C)(C)CCOCn1nc(S(=O)(=O)Cl)nc1Nc1c2c(cc3c1CCC3)CCC2 ; NCCN1CCCC1=O ; O=C1CCCN1CCNS(=O)(=O)c1nnc(Nc2c3c(cc4c2CCC4)CCC3)[nH]1]

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 1-(2-aminoethyl)pyrrolidin-2-one and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) to afford the title compound (17 mg, 17% yield) as a sticky yellow gum.

LCMS m/z 431.0 (M+H)⁺ (ES⁺).

¹H NMR (DMSO-d₆). δ 12.90 (s, 1H), 8.85 (s, 1H), 7.96-7.94 (m, 1H), 6.95 (s, 1H), 3.33 (t, J=7.0 Hz, 2H), 3.24 (t, J=6.5 Hz, 2H), 3.07 (q, J=6.4 Hz, 2H), 2.82 (t, J=7.4 Hz, 4H), 2.63 (t, J=7.4 Hz, 4H), 2.18 (t, J=8.1 Hz, 2H), 1.97 (p, J=7.4 Hz, 4H), 1.89 (p, J=7.4 Hz, 2H).

### Example 115: N-(2-(5-((1,2,3,5,6,7-hexahydro-s-indacen-4-yl)amino)-4H-1,2,4-triazole-3-sulfonamido)ethyl)acetamide

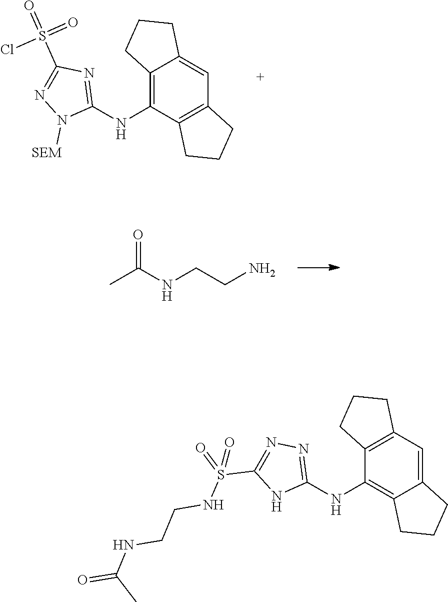

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from N-(2-aminoethyl)acetamide and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) to afford the title compound (14 mg, 15% yield) as a pale yellow powder.

LCMS m/z 405.3 (M+H)⁺ (ES⁺).

¹H NMR (DMSO-d₆). δ12.96 (br s, 1H), 8.85 (s, 1H), 7.90-7.85 (m, 2H), 6.95 (s, 1H), 3.14-3.04 (m, 2H), 3.00-2.93 (m, 2H), 2.82 (t, J=7.3 Hz, 4H), 2.63 (t, J=7.3 Hz, 4H), 1.97 (p, J=7.4 Hz, 4H), 1.77 (s, 3H).

### Example 16:5-((1,2,3,5,6,7-hexahydro-s-indacen-4-yl)amino)-N-(3-hydroxy-3-methylbutyl)-4H-1,2,4-triazole-3-sulfonamide

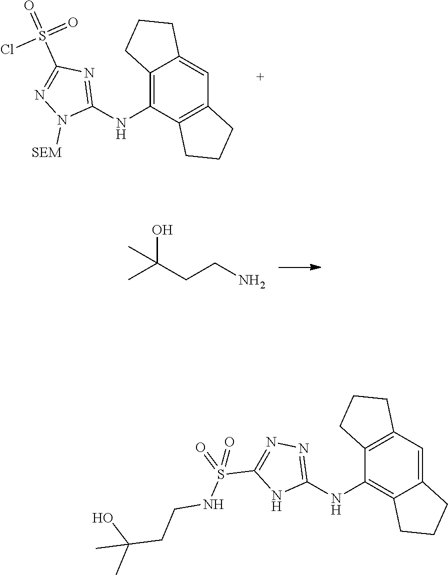

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 4-amino-2-methylbutan-2-ol and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) to afford the title compound (14 mg, 16% yield) as a flocculent white solid.

LCMS m/z 406.3 (M+H)⁺ (ES⁺).

¹H NMR (DMSO-d₆). δ12.88 (s, 1H), 8.82 (s, 1H), 7.63 (t, J=5.6 Hz, 1H), 6.94 (s, 1H), 4.29 (s, 1H), 3.12-2.94 (m, 2H), 2.82 (t, J=7.4 Hz, 4H), 2.64 (t, J=7.3 Hz, 4H), 1.97 (p, J=7.5 Hz, 4H), 1.62-1.43 (m 2H), 1.04 (s, 6H).

### Example 17: N-(1,2,3,5,6,7-hexahydro-s-indacen-4-yl)-5-((4-isopropylpiperazin-1-yl)sulfonyl)-4H-1,2,4-triazol-3-amine

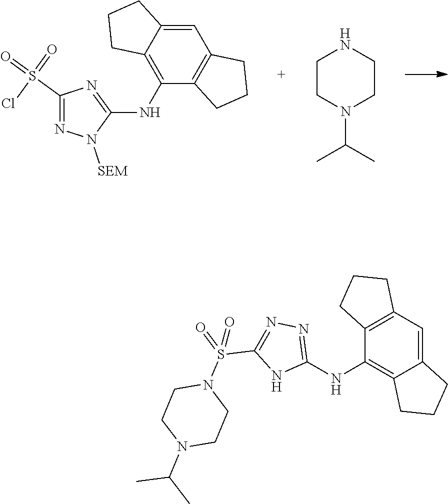

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 1-isopropylpiperazine and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B⁴) to afford the title compound (40 mg, 43% yield) as a flocculent white solid.

LCMS m/z 431.3 (M+H)⁺ (ES⁺).

¹H NMR (DMSO-d₆): δ 13.09 (br s, 1H), 8.95 (s, 1H), 6.97 (s, 1H), 3.16-3.14 (m, 4H), 2.83 (t, J=7.4 Hz, 4H), 2.72-2.64 (m, 5H), 1.98 (p, J=7.4 Hz, 4H), 0.96 (d, J=6.5 Hz, 6H). Four protons obscured by water peak.

### Example 118: 1-((5-((1,2,3,5,6,7-hexahydro-s-indacen-4-yl)amino)-4H-1,2,4-triazol-3-yl)sulfonyl)-N,N-dimethylpiperidin-4-amine

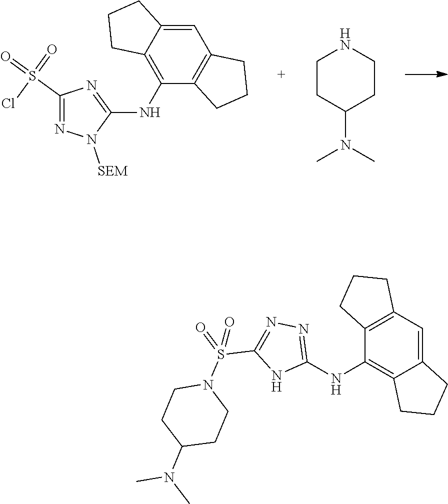

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from N,N-dimethylpiperidin-4-amine and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) to afford the title compound (20 mg, 22% yield) as a flocculent white solid.

LCMS m/z 431.3 (M+H)⁺ (ES⁺).

¹H NMR (DMSO-d₆): δ 9.01 (s, 1H), 6.95 (s, 1H), 3.68-3.64 (m, 2H), 2.84-2.74 (m, 6H), 2.63 (t, J=7.3 Hz, 4H), 2.33-2.24 (m, 1H), 2.20 (s, 6H), 1.97 (p, J=7.4 Hz, 4H), 1.80-1.77 (m 2H), 1.47-1.39 (m 2H). One exchangeable proton not observed.

### Example 119: 5-((3-((dimethylamino)methyl)pyrrolidin-1-yl)sulfonyl)-N-(1,2,3,5,6,7-hexahydro-s-indacen-4-yl)-4H-1,2,4-triazol-3-amine

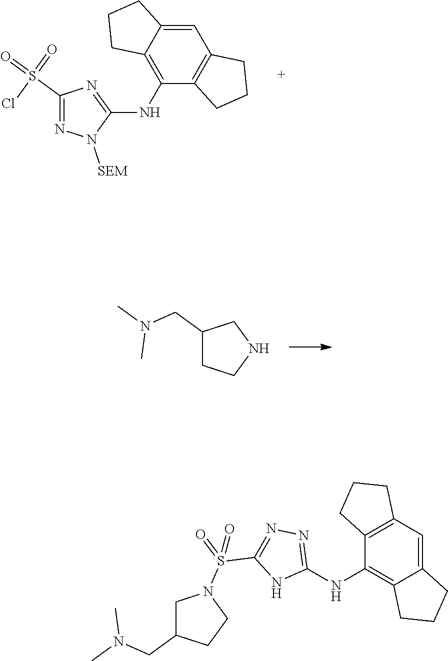

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from N,N-dimethyl-1-(pyrrolidin-3-yl)methanamine and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) to afford the title compound (32 mg, 35% yield) as a flocculent white solid.

LCMS m/z 431.1 (M+H)⁺ (ES⁺).

¹H NMR (DMSO-d₆): δ 8.95 (s, 1H), 6.95 (s, 1H), 2.97 (dd, J=10.2, 7.1 Hz, 1H), 2.82 (t, J=7.4 Hz, 4H), 2.63 (t, J=7.4 Hz, 4H), 2.26 (app sept, J=7.2 Hz, 1H), 2.10 (s, 6H), 1.97 (p, J=7.4 Hz, 4H), 1.92-1.82 (m, 1H), 1.56-1.41 (m, 1H). One exchangeable proton not observed. Five protons obscured by water peak.

### Example 120: N-(azetidin-3-yl)-5-((1,2,3,5,6,7-hexahydro-s-indacen-4-yl)amino)-N-methyl-4H-1,2,4-triazole-3-sulfonamide

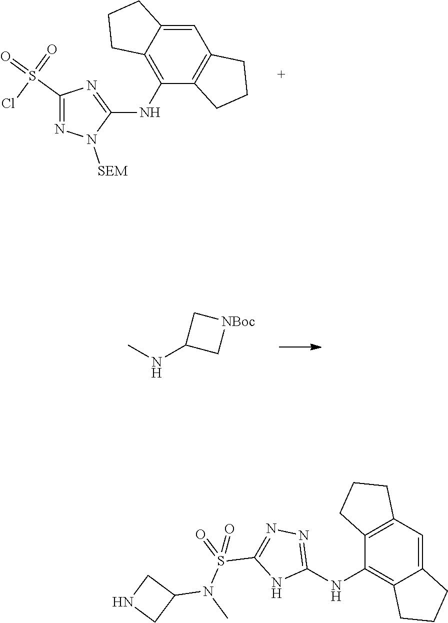 [SMILES: C[Si](C)(C)CCOCn1nc(S(=O)(=O)Cl)nc1Nc1c2c(cc3c1CCC3)CCC2 ; CNC1CN(C(=O)OC(C)(C)C)C1 ; CN(C1CNC1)S(=O)(=O)c1nnc(Nc2c3c(cc4c2CCC4)CCC3)[nH]1]

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from tert-butyl 3-(methylamino)azetidine-1-carboxylate and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) to afford the title compound (9 mg, 11% yield) as a flocculent white solid.

LCMS m/z 389.1 (M+H)⁺ (ES⁺).

¹H NMR (DMSO-d₆) δ 9.15-9.05 (m, 1H), 8.30-8.26 (m, 1H), 6.93 (s, 1H), 4.63-4.56 (m, 1H), 3.84-3.76 (m, 2H), 3.62-3.55 (m, 2H), 2.85-2.80 (m, 7H), 2.63 (t, J=7.3 Hz, 4H), 1.97 (p, J=7.4 Hz, 4H). One exchangeable proton not observed.

### Example 121: 5-((1,2,3,5,6,7-hexahydro-s-indacen-4-yl)amino)-N-((5-oxopyrrolidin-2-yl)methyl)-4H-1,2,4-triazole-3-sulfonamide

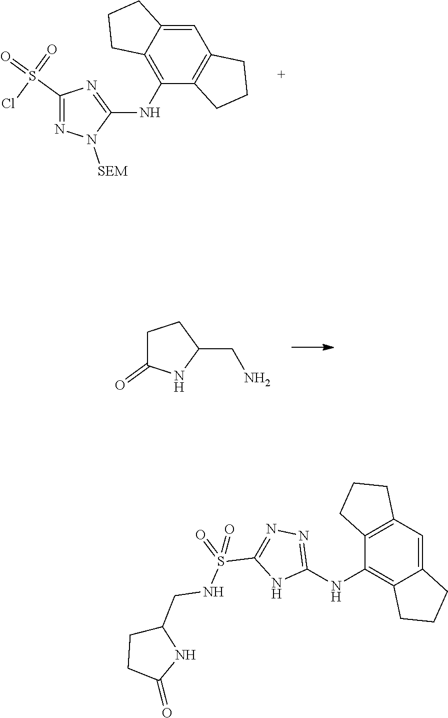 [SMILES: C[Si](C)(C)CCOCn1nc(S(=O)(=O)Cl)nc1Nc1c2c(cc3c1CCC3)CCC2 ; NCC1CCC(=O)N1 ; O=C1CCC(CNS(=O)(=O)c2nnc(Nc3c4c(cc5c3CCC5)CCC4)[nH]2)N1]

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 5-(aminomethyl)pyrrolidin-2-one and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) to afford the title compound (11 mg, 11% yield) as a sticky yellow gum.

LCMS m/z 417.3 (M+H)⁺ (ES⁺).

¹H NMR (DMSO-d₆): δ 12.97 (s, 1H), 8.87 (s, 1H), 8.00-7.98 (m, 1H), 7.53 (s, 1H), 6.95 (s, 1H), 3.55 (p, J=6.3 Hz, 1H), 3.05-2.97 (m, 1H), 2.94-2.86 (m, 1H), 2.82 (t, J=7.4 Hz, 4H), 2.63 (t, J=7.3 Hz, 4H), 2.16-2.01 (m, 3H), 2.00-1.92 (m, 4H), 1.76-1.65 (m, 1H).

### Example 122: N-(1,2,3,5,6,7-hexahydro-s-indacen-4-yl)-5-(pyrrolidin-1-ylsulfonyl)-4H-1,2,4-triazol-3-amine

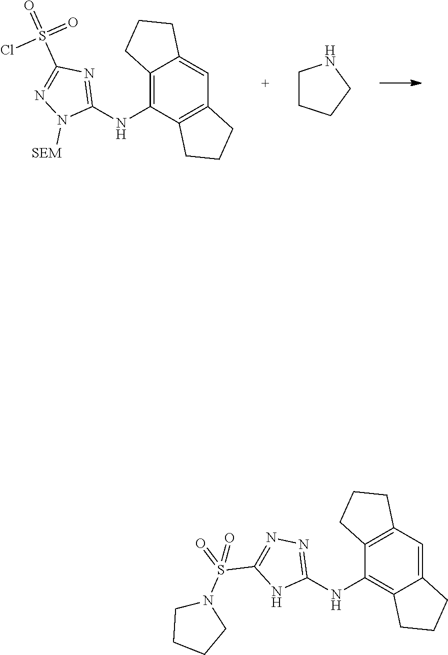 [SMILES: C[Si](C)(C)CCOCn1nc(S(=O)(=O)Cl)nc1Nc1c2c(cc3c1CCC3)CCC2 ; C1CCNC1 ; O=S(=O)(c1nnc(Nc2c3c(cc4c2CCC4)CCC3)[nH]1)N1CCCC1]

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from pyrrolidine and 5-((1,2,3,5,6,7-hexahydro-s-indacen-4-yl)amino)-1-((2-(trimethylsilyl)-ethoxy)methyl)-1H-1,2,4-triazole-3-sulfonyl chloride (Intermediate B4) to afford the title compound (10 mg, 12% yield) as a white solid.

LCMS m/z 374.3 (M+H)⁺ (ES⁺).

¹H NMR (DMSO-d₆): δ 13.02 (s, 1H), 8.90 (s, 1H), 6.96 (s, 1H), 3.39-3.24 (m, 4H), 2.83 (t, J=7.4 Hz, 4H), 2.63 (t, J=7.3 Hz, 4H), 1.97 (p, J=7.4 Hz, 4H), 1.79-1.73 (m, 4H).

### Example 123:1-((5-((1,2,3,5,6,7-hexahydro-s-indacen-4-yl)amino)-4H-1,2,4-triazol-3-yl)sulfonyl)pyrrolidin-3-ol

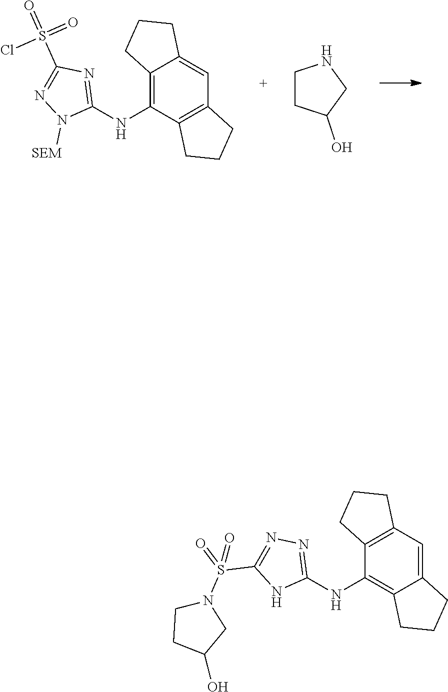 [SMILES: C[Si](C)(C)CCOCn1nc(S(=O)(=O)Cl)nc1Nc1c2c(cc3c1CCC3)CCC2 ; OC1CCNC1 ; O=S(=O)(c1nnc(Nc2c3c(cc4c2CCC4)CCC3)[nH]1)N1CCC(O)C1]

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from pyrrolidin-3-ol and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B⁴) to afford the title compound (8 mg, 9% yield) as a yellow gum.

LCMS m/z 390.5 (M+H)⁺ (ES⁺).

¹H NMR (DMSO-d₆): δ 13.01 (s, 1H), 8.89 (s, 1H), 6.96 (s, 1H), 5.03 (s, 1H), 4.23 (t, J=4.1 Hz, 1H), 3.44-3.38 (m, 3H), 3.11 (dd, J=10.4, 2.7 Hz, 1H), 2.83 (t, J=7.4 Hz, 4H), 2.63 (t, J=7.3 Hz, 4H), 1.97 (p, J=7.4 Hz, 4H), 1.84-1.76 (m, 1H), 1.75-1.68 (m, 1H).

### Example 124: N-(1,2,3,5,6,7-hexahydro-s-indacen-4-yl)-5-((4-(pyrrolidin-1-yl)piperidin-1-yl)sulfonyl)-4H-1,2,4-triazol-3-amine

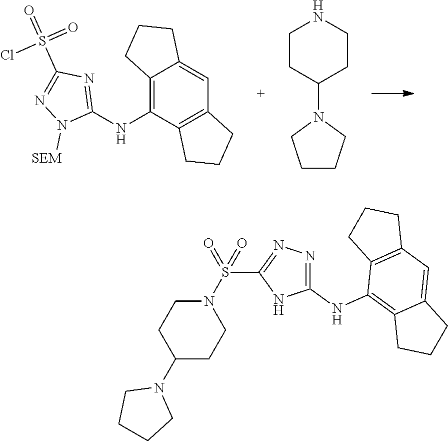 [SMILES: C[Si](C)(C)CCOCn1nc(S(=O)(=O)Cl)nc1Nc1c2c(cc3c1CCC3)CCC2 ; C1CCN(C2CCNCC2)C1 ; O=S(=O)(c1nnc(Nc2c3c(cc4c2CCC4)CCC3)[nH]1)N1CCC(N2CCCC2)CC1]

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 4-(pyrrolidin-1-yl)piperidine and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) to afford the title compound (7 mg, 7% yield) as a white solid.

LCMS m/z 457.4 (M+H)⁺ (ES⁺).

¹H NMR (DMSO-d₆): δ 9.05 (s, 1H), 6.95 (s, 1H), 3.61-3.53 (m, 2H), 2.86-2.78 (m, 6H), 2.66-2.56 (m, 8H), 2.31-2.21 (m, 1H), 1.97 (p, J=7.4 Hz, 4H), 1.92-1.86 (m, 2H), 1.73-1.67 (m, 4H), 1.52-1.41 (m, 2H). One exchangeable proton not observed.

### Example 125: N-(3-(dimethylamino)propyl)-5-((1,2,3,5,6,7-hexahydro-s-indacen-4-yl)amino)-4H-1,2,4-triazole-3-sulfonamide

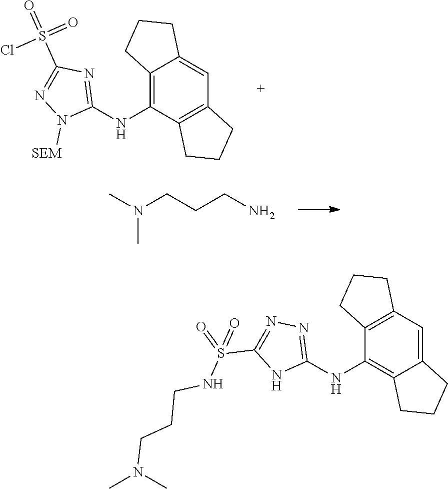

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from N¹,N¹-dimethylpropane-1,3-diamine and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) to afford the title compound (6 mg, 7% yield) as a white solid.

LCMS m/z 405.3 (M+H)⁺ (ES⁺).

¹H NMR (DMSO-d₆): δ 9.03 (s, 1H), 6.94 (s, 1H), 2.97 (t, J=7.0 Hz, 2H), 2.82 (t, J=7.4 Hz, 4H), 2.64 (t, J=7.3 Hz, 4H), 2.46 (t, J=7.3 Hz, 2H), 2.28 (s, 6H), 1.97 (p, J=7.4 Hz, 4H), 1.61 (p, J=7.1 Hz, 2H). Two exchangeable protons not observed.

### Example 126:5-((1,2,3,5,6,7-hexahydro-s-indacen-4-yl)amino)-N-(3-(pyrrolidin-1-yl)propyl)-4H-1,2,4-triazole-3-sulfonamide

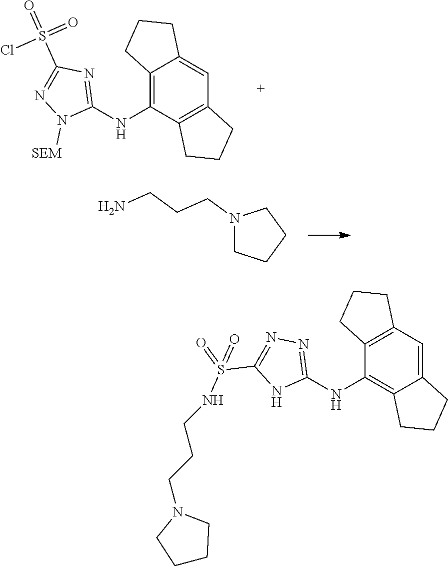

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 3-(pyrrolidin-1-yl)propan-1-amine and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) to afford the title compound (17 mg, 19% yield) as a white solid.

LCMS m/z 431.4 (M+H)⁺ (ES⁺).

¹H NMR (DMSO-d₆): δ 8.96 (s, 1H), 6.94 (s, 1H), 2.98 (t, J=7.0 Hz, 2H), 2.82 (t, J=7.4 Hz, 4H), 2.64 (t, J=7.3 Hz, 4H), 2.59-2.52 (m, 6H), 1.97 (p, J=7.4 Hz, 4H), 1.77-1.66 (m, 4H), 1.62 (p, J=7.1 Hz, 2H). Two exchangeable protons not observed.

### Example 127:N-(1,2,3,5,6,7-hexahydro-s-indacen-4-yl)-5-((3-morpholinopyrrolidin-1-yl)sulfonyl)-4H-1,2,4-triazol-3-amine

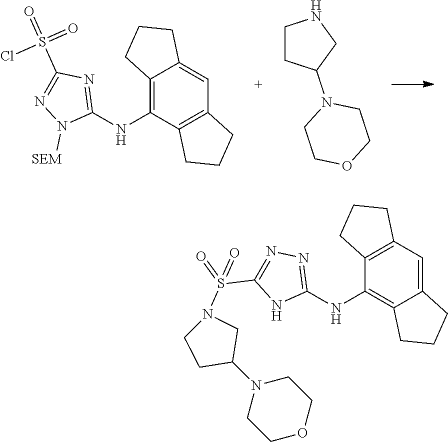

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 4-(pyrrolidin-3-yl)morpholine and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) to afford the title compound (20 mg, 19% yield) as a white solid.

LCMS m/z 459.3 (M+H)⁺ (ES⁺).

¹H NMR (DMSO-d₆): δ 13.10 (s, 1H), 8.93 (s, 1H), 6.96 (s, 1H), 3.59-3.47 (m, 5H), 3.48-3.42 (m, 1H), 3.33-3.24 (m, 1H), 3.11-2.98 (m, 1H), 2.83 (t, J=7.4 Hz, 4H), 2.72-2.57 (m, 5H), 2.43-2.20 (m, 4H), 2.08-1.90 (m, 5H), 1.76-1.55 (m, 1H).

### Example 128: 5-((1,2,3,5,6,7-hexahydro-s-indacen-4-yl)amino)-N-((1-methylpiperidin-3-yl)methyl)-4H-1,2,4-triazole-3-sulfonamide

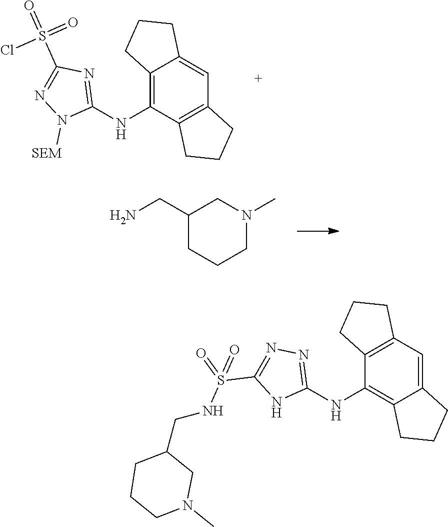

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from (1-methylpiperidin-3-yl)methanamine and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) to afford the title compound (15 mg, 16% yield) as a flocculent white solid.

LCMS m/z 431.3 (M+H)⁺ (ES⁺).

¹H NMR (DMSO-d₆): δ 9.10 (s, 1H), 7.92 (s, 1H), 6.93 (s, 1H), 3.08-2.92 (m, 2H), 2.91-2.77 (m, 6H), 2.65 (t, J=7.3 Hz, 4H), 2.44 (s, 3H), 2.39-2.30 (m, 1H), 2.13 (t, J=11.1 Hz, 1H), 1.96 (p, J=7.4 Hz, 4H), 1.87-1.75 (m, 1H), 1.73-1.60 (m, 2H), 1.57-1.45 (m, 1H), 1.07-0.89 (m, 1H). One exchangeable proton not observed.

### Example 129: 5-((1,2,3,5,6,7-hexahydro-s-indacen-4-yl)amino)-N-(2-morpholinoethyl)-4H-1,2,4-triazole-3-sulfonamide

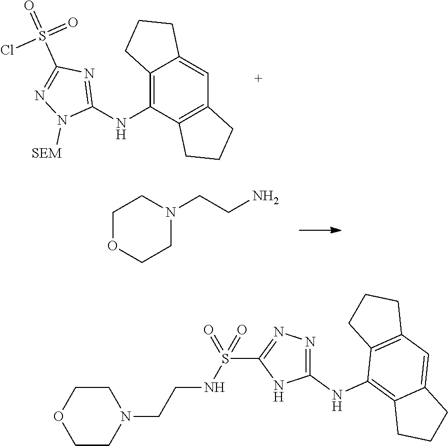

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 2-morpholinoethanamine and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B⁴) to afford the title compound (11 mg, 12% yield) as a flocculent white solid.

LCMS m/z 433.3 (M+H)⁺ (ES⁺).

¹H NMR (DMSO-d₆). δ 12.87 (s, 1H), 8.83 (s, 1H), 7.74 (s, 1H) 6.95 (s, 1H), 3.53 (t, J=4.6 Hz, 4H), 3.07 (q, J=6.5 Hz, 2H), 2.82 (t, J=7.4 Hz, 4H), 2.63 (t, J=7.3 Hz, 4H), 2.44-2.27 (m, 6H), 1.97 (p, J=7.4 Hz, 4H).

### Example 10: 1-((5-((1,2,3,5,6,7-hexahydro-s-indacen-4-yl)amino)-4H-1,2,4-triazol-3-yl)sulfonyl)-N-methylpiperidin-4-amine

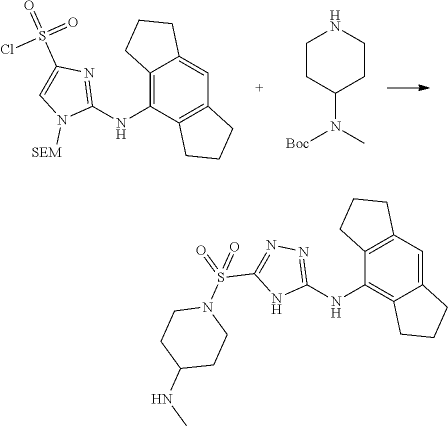 [SMILES: C[Si](C)(C)CCOCn1cc(S(=O)(=O)Cl)nc1Nc1c2c(cc3c1CCC3)CCC2 ; CN(C(=O)OC(C)(C)C)C1CCNCC1 ; CNC1CCN(S(=O)(=O)c2nnc(Nc3c4c(cc5c3CCC5)CCC4)[nH]2)CC1]

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from tert-butyl methyl(piperidin-4-yl)carbamate and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) to afford the title compound (13 mg, 15% yield) as a white solid.

LCMS m/z 417.3 (M+H)⁺ (ES⁺).

¹H NMR (DMSO-d₆): δ 9.22 (s, 1H), 6.94 (s, 1H), 3.66-3.58 (m, 2H), 2.89-2.73 (m, 6H), 2.70-2.64 (m, 5H), 2.37 (s, 3H), 2.03-1.89 (m, 7H), 1.46-1.33 (m, 2H). One exchangeable proton not observed.

### Example 131: 5-((1,2,3,5,6,7-hexahydro-s-indacen-4-yl)amino)-N-(2-(4-methylpiperazin-1-yl)ethyl)-4H-1,2,4-triazole-3-sulfonamide

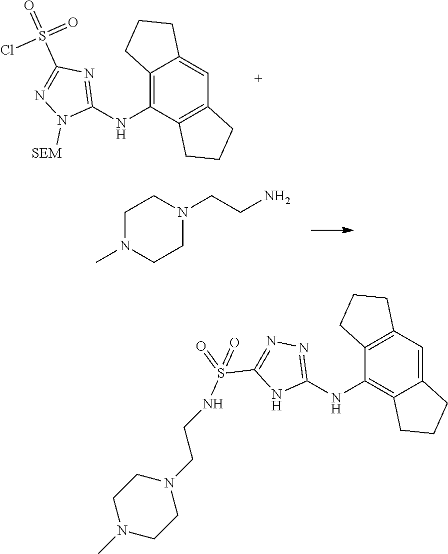

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 2-(4-methylpiperazin-1-yl)ethanamine and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) to afford the title compound (8 mg, 8% yield) as a white solid.

LCMS m/z 446.3 (M+H)⁺ (ES⁺).

¹H NMR (DMSO-d₆): δ 8.94 (s, 1H), 7.68 (s, 1H), 6.95 (s, 1H), 3.04 (t, J=7.3 Hz, 2H), 2.83 (t, J=7.4 Hz, 4H), 2.65 (t, J=7.3 Hz, 4H), 2.45-2.27 (m, 10H), 2.20 (s, 3H), 1.97 (p, J=7.4 Hz, 4H). One exchangeable proton not observed.

### Example 132:5-((1,2,3,5,6,7-hexahydro-s-indacen-4-yl)amino)-N-(piperidin-2-ylmethyl)-4H-1,2,4-triazole-3-sulfonamide

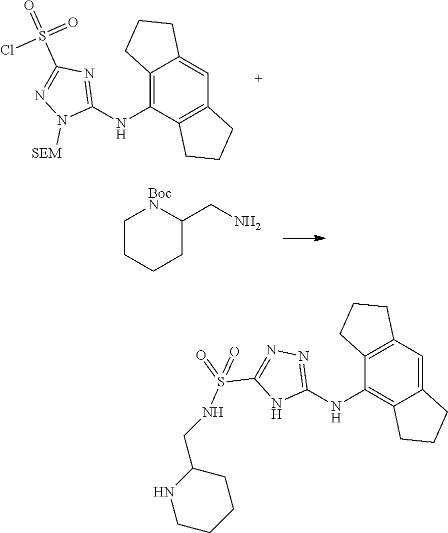

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from tert-butyl 2-(aminomethyl)piperidine-1-carboxylate and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) to afford the title compound (15 mg, 17% yield) as a flocculent white solid.

LCMS m/z 417.3 (M+H)⁺ (ES⁺).

¹H NMR (DMSO-d₆): δ 9.07 (s, 1H), 6.93 (s, 1H), 3.04-2.94 (m, 3H), 2.81 (t, J=7.4 Hz, 4H), 2.77-2.70 (m, 1H), 2.64 (t, J=7.3 Hz, 4H), 2.59-2.53 (m, 1H), 1.96 (p, J=7.4 Hz, 4H), 1.72-1.61 (m, 2H), 1.59-1.51 (m, 1H), 1.43-1.24 (m, 3H), 1.15-1.04 (m, 1H). Two exchangeable protons not observed.

### Example 133: N-(1,2,3,5,6,7-hexahydro-s-indacen-4-yl)-5-((3-(piperidin-1-yl)azetidin-1-yl)sulfonyl)-4H-1,2,4-triazol-3-amine

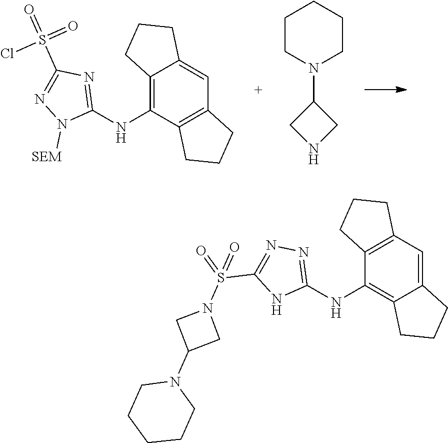 [SMILES: C[Si](C)(C)CCOCn1nc(S(=O)(=O)Cl)nc1Nc1c2c(cc3c1CCC3)CCC2 ; C1CCN(C2CNC2)CC1 ; O=S(=O)(c1nnc(Nc2c3c(cc4c2CCC4)CCC3)[nH]1)N1CC(N2CCCCC2)C1]

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 1-(azetidin-3-yl)piperidine dihydrochloride and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) to afford the title compound (11 mg, 11% yield) as a sticky yellow gum.

LCMS m/z 443.3 (M+H)⁺ (ES⁺).

¹H NMR (DMSO-d₆). δ13.28 (s, 1H), 9.02 (s, 1H), 6.98 (s, 1H), 4.10-3.97 (m, 5H), 2.83 (t, J=7.4 Hz, 4H), 2.67 (t, J=7.3 Hz, 4H), 1.98 (p, J=7.4 Hz, 4H), 1.68-1.28 (m, 6H). Four protons obscured by water peak.

### Example 134: 5-((1,2,3,5,6,7-hexahydro-s-indacen-4-yl)amino)-N-phenethyl-4H-1,2,4-triazole-3-sulfonamide

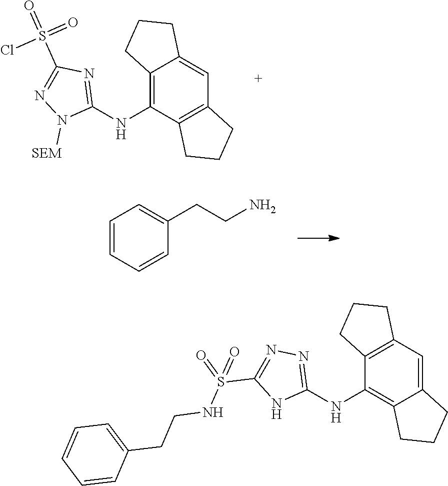 [SMILES: C[Si](C)(C)CCOCn1nc(S(=O)(=O)Cl)nc1Nc1c2c(cc3c1CCC3)CCC2 ; NCCc1ccccc1 ; O=S(=O)(NCCc1ccccc1)c1nnc(Nc2c3c(cc4c2CCC4)CCC3)[nH]1]

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 2-phenylethanamine and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B⁴) to afford the title compound (11 mg, 12% yield) as a flocculent white solid.

LCMS m/z 424.3 (M+H)⁺ (ES⁺).

¹H NMR (DMSO-d₆). δ 12.90 (s, 1H), 8.84 (s, 1H), 7.95 (t, J=5.8 Hz, 1H), 7.31-7.24 (m, 2H), 7.23-7.13 (m, 3H), 6.94 (s, 1H), 3.20-3.11 (m, 2H), 2.80 (t, J=7.4 Hz, 4H), 2.77-2.69 (m, 2H), 2.63 (t, J=7.3 Hz, 4H), 1.94 (p, J=7.4 Hz, 4H).

### Example 135: N-(1,2,3,5,6,7-hexahydro-s-indacen-4-yl)-5-((4-(4-methylpiperazin-1-yl)piperidin-1-yl)sulfonyl)-4H-1,2,4-triazol-3-amine

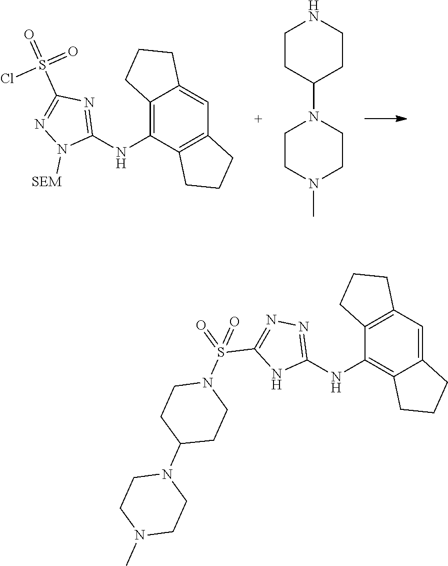 [SMILES: C[Si](C)(C)CCOCn1nc(S(=O)(=O)Cl)nc1Nc1c2c(cc3c1CCC3)CCC2 ; CN1CCN(C2CCNCC2)CC1 ; CN1CCN(C2CCN(S(=O)(=O)c3nnc(Nc4c5c(cc6c4CCC6)CCC5)[nH]3)CC2)CC1]

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 1-methyl-4-(piperidin-4-yl)piperazine and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) to afford the title compound (5 mg, 5% yield) as a sticky yellow gum.

LCMS m/z 486.4 (M+H)⁺ (ES⁺).

¹H NMR (DMSO-d₆): δ 9.02 (s, 1H), 6.96 (s, 1H), 3.67 (app d, J=12.6 Hz, 2H), 2.83 (t, J=7.4 Hz, 4H), 2.75 (td, J=12.3, 2.5 Hz, 2H), 2.64 (t, J=7.3 Hz, 4H), 2.49-2.36 (m, 8H), 2.31-2.24 (m, 1H), 2.22 (s, 3H), 1.98 (p, J=7.4 Hz, 4H), 1.81-1.73 (m, 2H), 1.48-1.37 (m 2H). One exchangeable proton not observed.

### Example 136: N-(1,2,3,5,6,7-hexahydro-s-indacen-4-yl)-5-((3-(pyrrolidin-1-yl)azetidin-1-yl)sulfonyl)-4H-1,2,4-triazol-3-amine

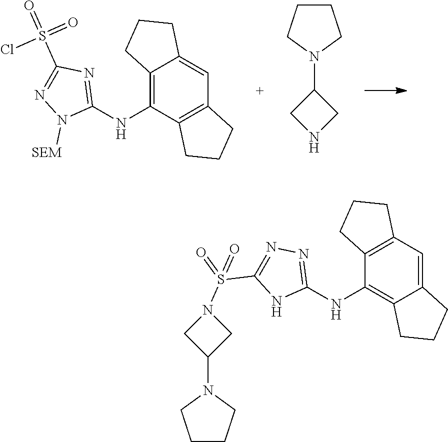 [SMILES: C[Si](C)(C)CCOCn1nc(S(=O)(=O)Cl)nc1Nc1c2c(cc3c1CCC3)CCC2 ; C1CCN(C2CNC2)C1 ; O=S(=O)(c1nnc(Nc2c3c(cc4c2CCC4)CCC3)[nH]1)N1CC(N2CCCC2)C1]

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 1-(azetidin-3-yl)pyrrolidine dihydrochloride and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) to afford the title compound (14 mg, 15% yield) as a white solid.

LCMS m/z 429.3 (M+H)⁺ (ES⁺).

¹H NMR (DMSO-d₆): δ 9.01 (s, 1H), 6.97 (s, 1H), 3.90 (t, J=7.9 Hz, 2H), 3.83 (dd, J=8.4, 6.2 Hz, 2H), 3.37 (p, J=6.8 Hz, 1H), 2.83 (t, J=7.4 Hz, 4H), 2.66 (t, J=7.3 Hz, 4H), 2.40-2.26 (m, 4H), 1.98 (p, J=7.4 Hz, 4H), 1.74-1.57 (m, 4H). One exchangeable proton not observed.

### Example 147: N-(1,2,3,5,6,7-hexahydro-s-indacen-4-yl)-5-((6-methyl-2,6-diazaspiro[3.3]heptan-2-yl)sulfonyl)-4H-1,2,4-triazol-3-amine

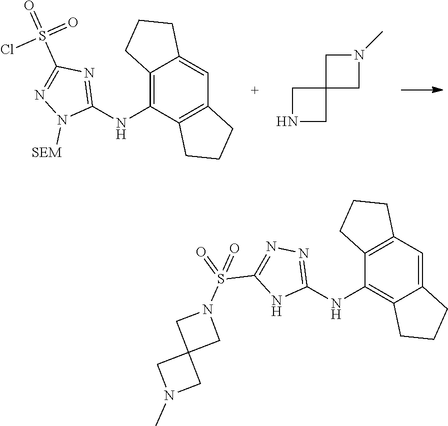

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 2-methyl-2,6-diazaspiro[3.3]heptane bis(2,2,2-trifluoroacetate) and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) to afford the title compound (5 mg, 6% yield) as a flocculent white solid.

LCMS m/z 415.2 (M+H)⁺ (ES⁺).

¹H NMR (DMSO-d₆): δ 9.03 (s, 1H), 6.98 (s, 1H), 4.02 (s, 4H), 3.31 (s, 4H), 2.84 (t, J=7.4 Hz, 4H), 2.65 (t, J=7.3 Hz, 4H), 2.26 (s, 3H), 1.98 (p, J=7.5 Hz, 4H). One exchangeable proton not observed.

### Example 138: N-(1,2,3,5,6,7-hexahydro-s-indacen-4-yl)-5-((5-methylhexahydropyrrolo[3,4-c]pyrrol-2 (1H)-yl)sulfonyl)-4H-1,2,4-triazol-3-amine

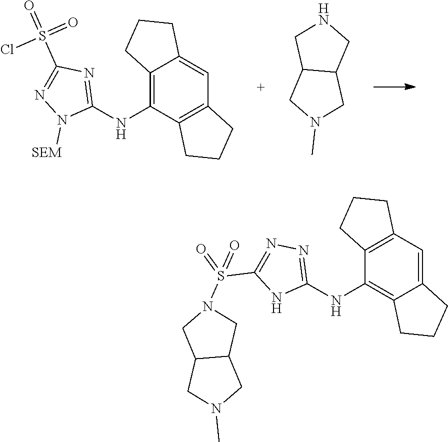

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 2-methyloctahydropyrrolo[3,4-c]pyrrole and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) to afford the title compound (12 mg, 13% yield) as a flocculent white solid.

LCMS m/z 429.3 (M+H)⁺ (ES⁺).

¹H NMR (DMSO-d₆): δ 8.94 (s, 1H), 6.96 (s, 1H), 3.44-3.37 (m, 2H), 3.06-2.99 (m, 2H), 2.82 (t, J=7.4 Hz, 4H), 2.77 (br s, 2H), 2.63 (t, J=7.4 Hz, 4H), 2.59-2.52 (m, 2H), 2.48-2.40 (m, 2H), 2.29 (s, 3H), 2.01-1.91 (m, 4H). One exchangeable proton not observed.

### Example 139: 1-((5-((1,2,3,5,6,7-hexahydro-s-indacen-4-yl)amino)-4H-1,2,4-triazol-3-yl)sulfonyl)-N,N-dimethylpyrrolidine-3-carboxamide

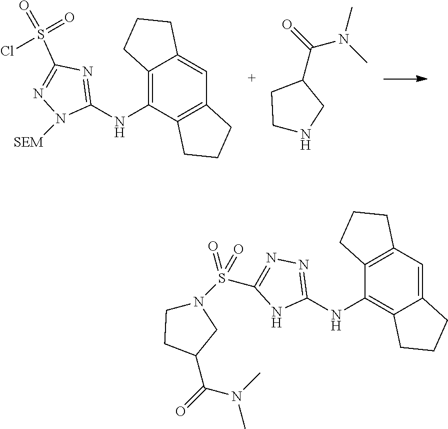

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and N,N-dimethyl-pyrrolidine-3-carboxamide to afford the title compound (5 mg, 3% yield) as an off white solid.

LCMS m/z 445.3 (M+H)⁺ (ES⁺).

¹H NMR (DMSO-d₆) δ 13.05 (s, 1H), 8.92 (s, 1H), 6.96 (s, 1H), 3.60-3.53 (m, 1H), 3.48-3.41 (m, 2H), 3.36-3.25 (m, 2H), 2.95 (s, 3H), 2.86-2.78 (m, 7H), 2.64 (t, J=7.3 Hz, 4H), 2.06-1.93 (m, 5H), 1.90-1.80 (m, 1H).

### Example 140: 1-((5-((1,2,3,5,6,7-hexahydro-s-indacen-4-yl)amino)-4H-1,2,4-triazol-3-yl)sulfonyl)-N-methylpyrrolidine-3-carboxamide

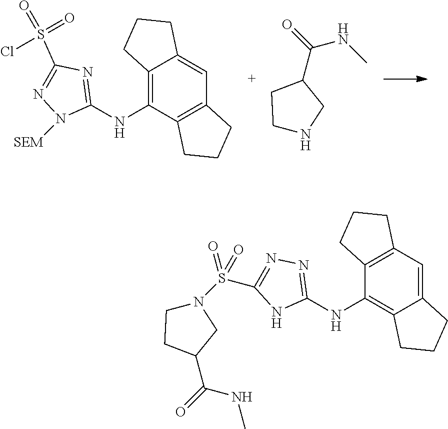

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and N-methylpyrrolidine-3-carboxamide to afford the title compound (14 mg, 9% yield) as an off white solid.

LCMS m/z 431.5 (M+H)⁺ (ES⁺).

¹H NMR (DMSO-d₆) δ 13.04 (s, 1H), 8.91 (s, 1H), 7.88 (br. s, 1H), 6.96 (s, 1H), 3.54-3.48 (m, 1H), 3.48-3.42 (m, 1H), 3.35-3.28 (m, 2H), 2.92-2.86 (m, 1H), 2.83 (t, J=7.4 Hz, 4H), 2.64 (t, J=7.4 Hz, 4H), 2.57 (d, J=4.6 Hz, 3H), 2.02-1.93 (m, 5H), 1.92-1.83 (m, 1H).

### Example 141:5-((1,2,3,5,6,7-hexahydro-s-indacen-4-yl)amino)-N-((1-methylpiperidin-2-yl)methyl)-4H-1,2,4-triazole-3-sulfonamide

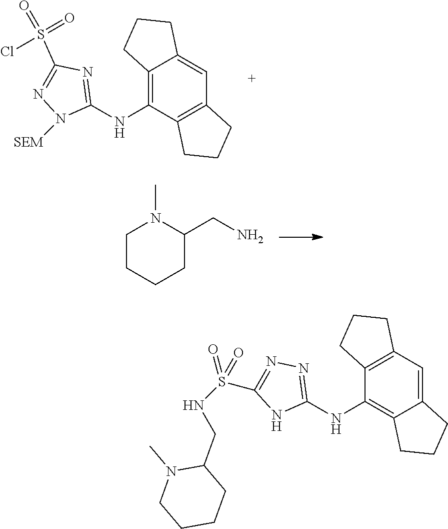

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and (1-methylpiperidin-2-yl)methanamine to afford the title compound (6 mg, 7% yield) as a pale white solid.

LCMS m/z 431.3 (M+H)⁺ (ES⁺).

¹H NMR (Methanol-d₄) δ 7.04 (s, 1H), 3.77-3.68 (m, 1H), 3.32-3.28 (m, 1H), 3.13-3.03 (m, 1H), 3.03-2.96 (m, 1H), 2.92 (t, J=7.5 Hz, 4H), 2.89-2.87 (m, 1H), 2.84 (s, 3H), 2.81-2.70 (m, 4H), 2.14-2.03 (m, 4H), 1.90-1.75 (m, 3H), 1.71-1.58 (m, 2H), 1.58-1.46 (m, 1H). 3 exchangeable protons not observed.

### Example 142: N-(1,2,3,5,6,7-hexahydro-s-indacen-4-yl)-5-((5-methylhexahydropyrrolo[3,4-b]pyrrol-1 (2H)-yl)sulfonyl)-4H-1,2,4-triazol-3-amine

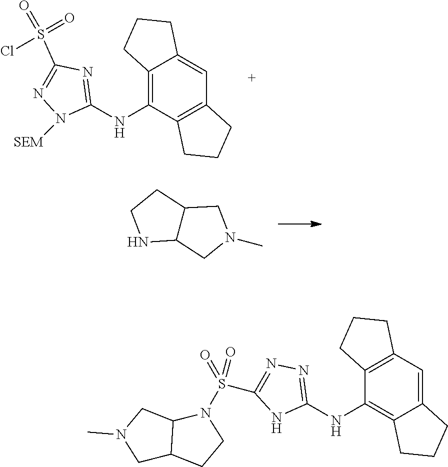

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 5-methyloctahydropyrrolo[3,4-b]pyrrole and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) to afford the title compound (23 mg, 24% yield) as a white solid.

LCMS m/z 429.3 (M+H)⁺ (ES⁺).

¹H NMR (DMSO-d₆): δ 8.95 (s, 1H), 6.95 (s, 1H), 4.20-4.08 (m, 1H), 3.43-3.35 (m, 1H), 3.35-3.28 (m, 1H), 2.87-2.77 (m, 5H), 2.73-2.65 (m, 1H), 2.63 (t, J=7.4 Hz, 4H), 2.30-2.20 (m, 2H), 2.19 (s, 3H), 1.97 (p, J=7.4 Hz, 4H), 1.86-1.75 (m, 1H), 1.71-1.64 (m, 1H). One exchangeable proton not observed. One proton obscured by solvent.

### Example 143: (S)-5-((3-((diethylamino)methyl)pyrrolidin-1-yl)sulfonyl)-N-(1,2,3,5,6,7-hexahydro-s-indacen-4-yl)-4H-1,2,4-triazol-3-amine

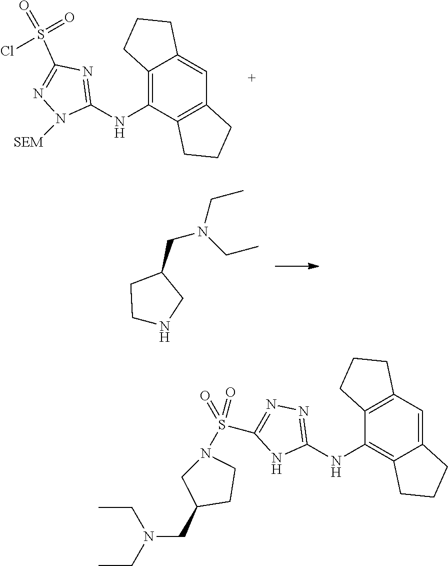

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from (R)—N-ethyl-N-(pyrrolidin-3-ylmethyl)ethanamine and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) to afford the title compound (22 mg, 22% yield) as a white solid.

LCMS m/z 459.4 (M+H)⁺ (ES⁺).

¹H NMR (DMSO-d₆): δ 8.96 (s, 1H), 6.95 (s, 1H), 3.44-3.37 (m, 2H), 3.35-3.28 (m, 1H), 2.95 (dd, J=10.1, 6.4 Hz, 1H), 2.82 (t, J=7.4 Hz, 4H), 2.63 (t, J=7.3 Hz, 4H), 2.45-2.34 (m, 4H), 2.29-2.17 (m, 3H), 1.97 (p, J=7.5 Hz, 4H), 1.91-1.83 (m, 1H), 1.55-1.44 (m, 1H), 0.89 (t, J=7.1 Hz, 6H). One exchangeable proton not observed.

### Example 144: (R)-5-((3-((diethylamino)methyl)pyrrolidin-1-yl)sulfonyl)-N-(1,2,3,5,6,7-hexahydro-s-indacen-4-yl)-4H-1,2,4-triazol-3-amine

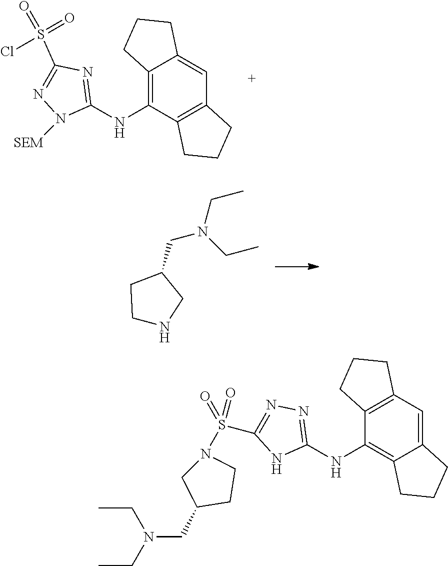

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from (S)—N-ethyl-N-(pyrrolidin-3-ylmethyl)ethanamine and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) to afford the title compound (17 mg, 17% yield) as a white solid.

LCMS m/z 459.4 (M+H)⁺ (ES⁺).

¹H NMR (DMSO-d₆): δ 8.96 (s, 1H), 6.95 (s, 1H), 3.44-3.37 (m, 2H), 3.34-3.28 (m, 1H), 2.95 (dd, J=10.2, 6.4 Hz, 1H), 2.82 (t, J=7.4 Hz, 4H), 2.63 (t, J=7.3 Hz, 4H), 2.44-2.35 (m, 4H), 2.27-2.17 (m, 3H), 1.97 (p, J=7.4 Hz, 4H), 1.91-1.83 (m, 1H), 1.53-1.45 (m, 1H), 0.89 (t, J=7.1 Hz, 6H). One exchangeable proton not observed.

### Example 145: 5-(7-oxa-2-azaspiro[35]nonan-2-ylsulfonyl)-N-(1,2,3,5,6,7-hexahydro-s-indacen-4-yl)-4H-1,2,4-triazol-3-amine

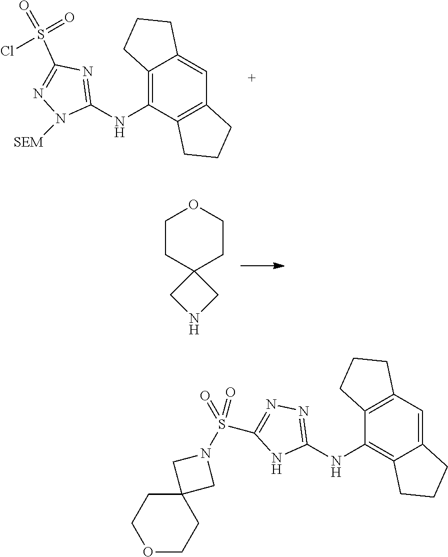 [SMILES: C[Si](C)(C)CCOCn1nc(S(=O)(=O)Cl)nc1Nc1c2c(cc3c1CCC3)CCC2 ; C1CC2(CCO1)CNC2 ; O=S(=O)(c1nnc(Nc2c3c(cc4c2CCC4)CCC3)[nH]1)N1CC2(CCOCC2)C1]

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 7-oxa-2-azaspiro[3.5]nonane hemioxalate and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) to afford the title compound (7 mg, 8% yield) as a white solid.

LCMS m/z 430.3 (M+H)⁺ (ES⁺).

¹H NMR (DMSO-d₆): δ 13-17 (s, 1H), 8.99 (s, 1H), 6.97 (s, 1H), 3.70 (s, 4H), 3.41 (t, J=5.2 Hz, 4H), 2.83 (t, J=7.4 Hz, 4H), 2.63 (t, J=7.3 Hz, 4H), 1.97 (p, J=7.4 Hz, 4H), 1.49 (t, J=5.2 Hz, 4H).

### Example 146: 5-(6-oxa-2-azaspiro[3.4]octan-2-ylsulfonyl)-N-(1,2,3,5,6,7-hexahydro-s-indacen-4-yl)-4H-1,2,4-triazol-3-amine

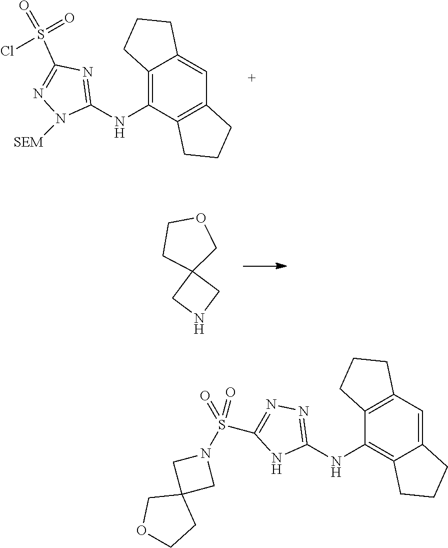 [SMILES: C[Si](C)(C)CCOCn1nc(S(=O)(=O)Cl)nc1Nc1c2c(cc3c1CCC3)CCC2 ; C1CC2(CNC2)CO1 ; O=S(=O)(c1nnc(Nc2c3c(cc4c2CCC4)CCC3)[nH]1)N1CC2(CCOC2)C1]

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 6-oxa-2-azaspiro[3.4]octane oxalate and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) to afford the title compound (16 mg, 18% yield) as a white solid.

LCMS m/z 416.3 (M+H)⁺ (ES⁺).

¹H NMR (DMSO-d₆): δ 13.21 (s, 1H), 9.02 (s, 1H), 6.98 (s, 1H), 3.98-3.86 (m, 4H), 3.62 (t, J=7.0 Hz, 2H), 3.56 (s, 2H), 2.83 (t, J=7.4 Hz, 4H), 2.66 (t, J=7.4 Hz, 4H), 1.98 (p, J=7.4 Hz, 4H), 1.92 (t, J=7.0 Hz, 2H).

### Example 147: (S)-5-((3-((dimethylamino)methyl)pyrrolidin-1-yl)sulfonyl)-N-(1,2,3,5,6,7-hexahydro-s-indacen-4-yl)-4H-1,2,4-triazol-3-amine

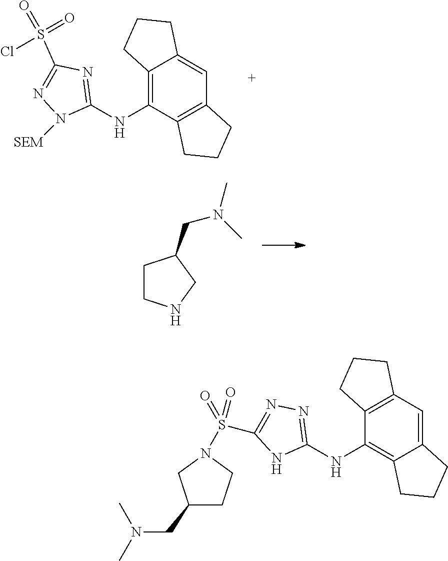

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from (R)—N,N-dimethyl-1-(pyrrolidin-3-yl)methanamine and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) to afford the title compound (16 mg, 17% yield) as a flocculent white solid.

LCMS m/z 431.1 (M+H)⁺ (ES⁺).

¹H NMR (DMSO-d₆): δ 8.97 (s, 1H), 6.96 (s, 1H), 3.47-3.37 (m, 2H), 3.34-3.27 (m, 1H), 2.97 (dd, J=10.2, 7.1 Hz, 1H), 2.83 (t, J=7.4 Hz, 4H), 2.64 (t, J=7.3 Hz, 4H), 2.26 (p, J=7.5 Hz, 1H), 2.10 (m, 7H), 1.97 (p, J=7.4 Hz, 4H), 1.93-1.86 (m, 1H), 1.54-1.42 (m, 1H). One exchangeable proton not observed. One aliphatic proton obscured by solvent.

### Example 148: (R)-5-((3-((dimethylamino)methyl)pyrrolidin-1-yl)sulfonyl)-N-(1,2,3,5,6,7-hexahydro-s-indacen-4-yl)-4H-1,2,4-triazol-3-amine

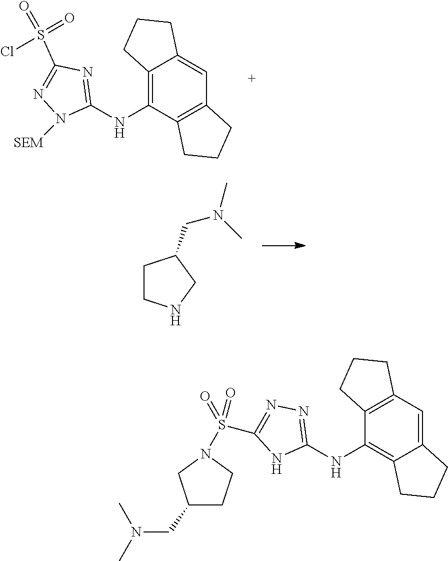

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from (S)—N,N-dimethyl-1-(pyrrolidin-3-yl)methanamine and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) to afford the title compound (16 mg, 17% yield) as a flocculent white solid.

LCMS m/z 431.3 (M+H)⁺ (ES⁺).

¹H NMR (DMSO-d₆): δ 9.16-8.82 (m, 1H), 6.96 (s, 1H), 3.50-3.35 (m, 2H), 3.36-3.28 (m, 1H), 2.99 (dd, J=10.2, 7.1 Hz, 1H), 2.83 (t, J=7.4 Hz, 4H), 2.63 (t, J=7.3 Hz, 4H), 2.34-2.27 (m, 1H), 2.22-2.18 (m, 7H), 2.03-1.88 (m, 5H), 1.55-1.45 (m, 1H). One exchangeable proton not observed. One aliphatic proton obscured by solvent.

### Example 149: 5-((2-((dimethylamino)methyl)morpholino)sulfonyl)-N-(1,2,3,5,6,7-hexahydro-s-indacen-4-yl)-4H-1,2,4-triazol-3-amine

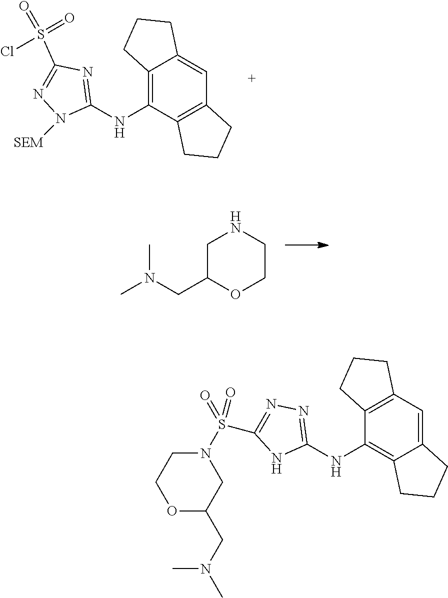

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from N,N-dimethyl-1-(morpholin-2-yl)methanamine and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) to afford the title compound (23 mg, 24% yield) as a flocculent white solid.

LCMS m/z 447.3 (M+H)⁺ (ES⁺).

¹H NMR (DMSO-d₆): δ 8.98 (s, 1H), 6.96 (s, 1H), 3.92-3.85 (m, 1H), 3.64-3.42 (m, 4H), 2.87-2.72 (m, 5H), 2.64 (t, J=7.4 Hz, 4H), 2.38 (br. s, 2H), 2.24-2.15 (m, 6H), 1.97 (p, J=7.4 Hz, 4H). One exchangeable proton not observed. One aliphatic proton obscured by solvent.

### Example 150: 5-(2,6-Diazaspiro[33]heptan-2-ylsulfonyl)-N-(1,2,3,5,6,7-hexahydro-s-indacen-4-yl)-4H-1,2,4-triazol-3-amine

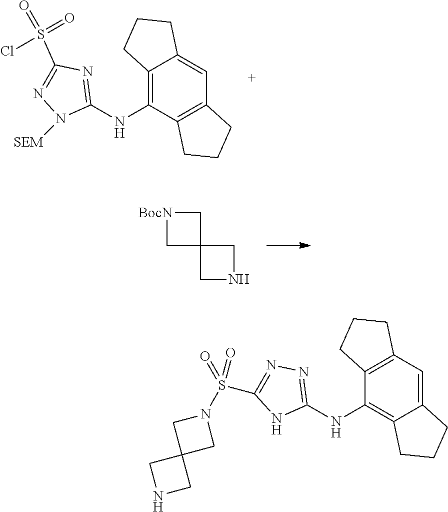 [SMILES: C[Si](C)(C)CCOCn1nc(S(=O)(=O)Cl)nc1Nc1c2c(cc3c1CCC3)CCC2 ; CC(C)(C)OC(=O)N1CC2(CNC2)C1 ; O=S(=O)(c1nnc(Nc2c3c(cc4c2CCC4)CCC3)[nH]1)N1CC2(CNC2)C1]

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from tert-butyl 2,6-diazaspiro[3.3]heptane-2-carboxylate hemioxalate and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) to afford the title compound (7 mg, 8% yield) as a flocculent white solid.

LCMS m/z 401.2 (M+H)⁺ (ES⁺).

¹H NMR (DMSO-d₆): δ 9.10 (s, 1H), 6.94 (s, 1H), 4.05 (s, 4H), 3.67 (s, 4H), 2.83 (t, J=7.4 Hz, 4H), 2.65 (t, J=7.1 Hz, 4H), 2.01-1.92 (m, 4H). Two exchangeable protons not observed.

### Example 151: 2-(1-((5-((1,2,3,5,6,7-hexahydro-s-indacen-4-yl)amino)-4H-1,2,4-triazol-3-yl)sulfonyl)azetidin-3-yl)propan-2-ol

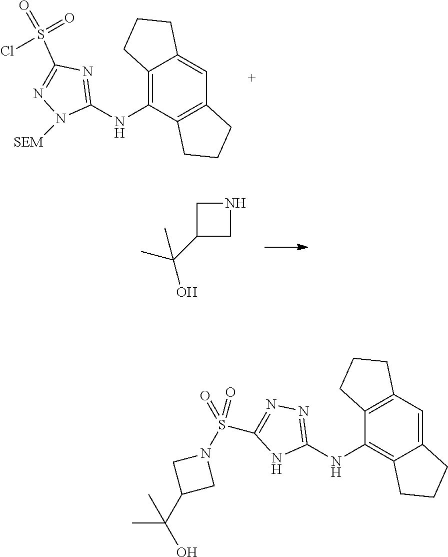

Prepared according to the general procedure of 5-((1,2,3,5,6,7-hexahydro-s-indacen-4-yl)amino)-N-(2-methoxyethyl)-4H-1,2,4-triazole-3-sulfonamide (Example 83) from 2-(azetidin-3-yl)propan-2-ol hydrochloride and 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) to afford the title compound (5 mg, 6% yield) as a white solid.

LCMS m/z 418.3 (M+H)⁺ (ES⁺).

¹H NMR (DMSO-d₆): δ 8.96 (s, 1H), 6.97 (s, 1H), 4.43 (s, 1H), 3.86-3.75 (m, 4H), 2.83 (t, J=7.4 Hz, 4H), 2.67 (t, J=7.3 Hz, 4H), 1.97 (p, J=7.4 Hz, 4H), 0.92 (s, 6H). One exchangeable proton not observed. One aliphatic proton obscured by solvent.

### Example 152: 1-(1-((5-(4-fluoro-2-isopropyl-6-(pyridin-4-yl)phenoxy)-4H-1,2,4-triazol-3-yl)sulfonyl)pyrrolidin-3-yl)-N,N-dimethylmethanamine

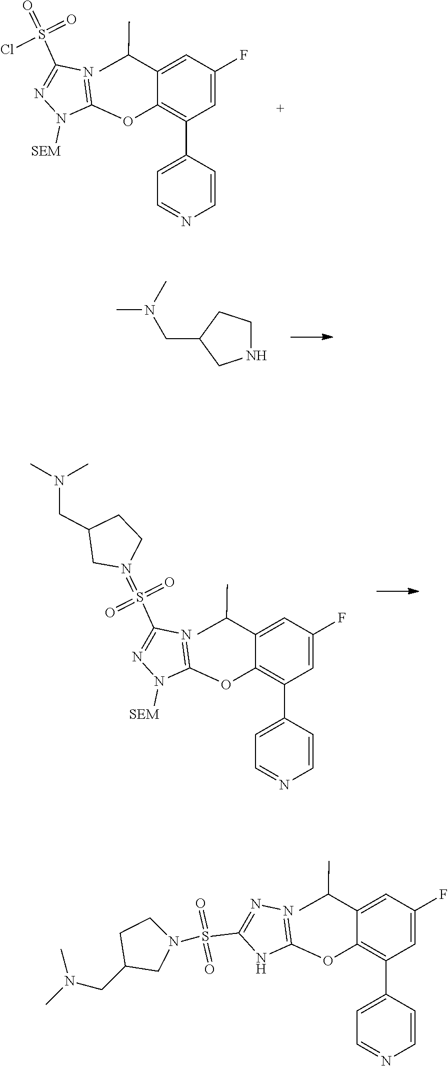

Step A: To a solution of 5-(4-fluoro-2-isopropyl-6-(pyridin-4-yl)phenoxy)-1-((2-(trimethylsilyl)ethoxy)methyl)-1H-1,2,4-triazole-3-sulfonyl chloride (Intermediate B⁸) (51 mg, 96.93 μmol, 1 eq) in DCM (5 mL) was added N,N-dimethyl-1-(pyrrolidin-3-yl) methanamine (12 mg, 96.93 μmol, 1 eq). The mixture was stirred at 25° C. for 10 minutes. The reaction mixture was concentrated in vacuum. The residue was purified by prep-TLC (SiO₂, dichloromethane:methanol, 10:1) to give 1-(1-((5-(4-fluoro-2-isopropyl-6-(pyridin-4-yl)phenoxy)-1-((2-(trimethylsilyl)ethoxy)methyl)-1H-1,2,4-triazol-3-yl)sulfonyl)pyrrolidin-3-yl)-N,N-dimethylmethanamine (40 mg, yield over three steps: 52.3%, 100% purity on LCMS) as a colourless oil.

¹H NMR (400 MHz, CDCl₃) δ 8.59 (d, 2H), 7.31 (d, 2H), 7.14 (dd, 1H), 6.94 (m, 1H), 5.31 (s, 2H), 3.65 (t, 2H), 3.50-3.37 (m, 2H), 3.25-3.14 (m, 1H), 3.11-3.04 (m, 1H), 3.01-2.95 (m, 1H), 2.31-2.20 (m, 2H), 2.19 (s, 6H), 2.15-2.11 (m, 1H), 1.93-1.83 (m, 1H), 1.55-1.48 (m, 1H), 1.24 (d, 6H), 0.93 (t, 2H), 0.04 (s, 9H).

LCMS: m/z 619.2 (M+H)⁺ (ES⁺).

Step B: To a solution of 1-(1-((5-(4-fluoro-2-isopropyl-6-(pyridin-4-yl)phenoxy)-1-((2-(trimethylsilyl)ethoxy)methyl)-1H-1,2,4-triazol-3-yl)sulfonyl)pyrrolidin-3-yl)-N,N-dimethylmethanamine (40 mg, 64.70 μmol, 1 eq) in DCM (1 mL) was added TFA (1 mL). The mixture was stirred at 25° C. for 12 hours. The reaction mixture was concentrated in vacuum. The residue was purified by prep-HPLC (column: Waters Xbridge C18, 150 mm*25 mm*5 μm; mobile phase: [A: water (0.05% ammonia hydroxide v/v), B: MeCN]; B %: 5%-35%,10 minutes) to give the title compound (11.32 mg, 35.8% yield, 100% purity on LCMS) as a white solid.

¹H NMR (400 MHz, CDCl₃) δ 8.52 (d, 2H), 7.40 (d, 2H), 7.09 (dd, 1H), 6.91 (dd, 1H), 3.69-3.60 (m, 1H), 3.44-3.37 (m, 1H), 3.32-3.26 (m, 1H), 3.20-315 (m, 1H), 2.78-2.69 (m, 2H), 2.55-2.50 (m, 1H), 2.45 (s, 6H), 2.39-2.29 (m, 1H), 1.95-1.86 (m, 1H), 1.49-1.35 (m, 1H), 1.20 (dd, 6H). One exchangeable proton not observed.

LCMS: m/z 489.4 (M+H)⁺ (ES⁺).

### Example 151: 1-(1-((5-(4-fluoro-2-isopropyl-6-(pyridin-4-yl)benzyl)-4H-1,2,4-triazol-3-yl)sulfonyl)pyrrolidin-3-yl)-N,N-dimethylmethanamine

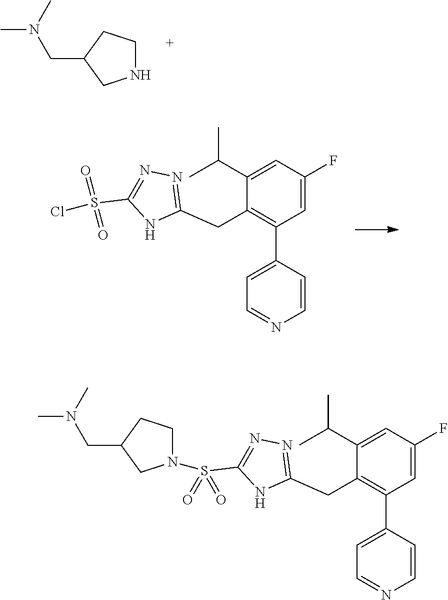

To a solution of 5-(4-fluoro-2-isopropyl-6-(pyridin-4-yl)benzyl)-4H-1,2,4-triazole-3-sulfonyl chloride (Intermediate R13) (120 mg, 303.91 μmol, 1 eq) in DCM (2 mL) were added DIPEA (393 mg, 3.04 mmol, 10 eq) and N,N-dimethyl-1-(pyrrolidin-3-yl)methanamine (58 mg, 455.87 μmol, 1.5 eq) in one portion. Then the reaction mixture was stirred at 25° C. for 1 hour. The mixture was quenched with water (20 mL) and extracted with DCM (3×20 mL). The combined organic phases were washed with brine (20 mL), dried over anhydrous Na₂SO₄, filtered and concentrated in vacuum. The residue was purified by prep-HPLC (column: Xtimate C18, 150 mm*25 mm*5 μm; mobile phase: [A: water (0.04% NH₃.H₂O+10 mM NH₄HCO), B: MeCN]; B %: 5%-35%, min) to give the title compound (36.09 mg, yield over two steps: 24.4% yield, 100% purity on LCMS) as a yellow solid.

¹H NMR (400 MHz, CDCl₃) δ 8.54 (dd, 2H), 7.22 (dd, 2H), 7.11 (dd, 1H), 6.78 (dd, 1H), 4.05 (s, 2H), 3.66-3.55 (m, 1H), 3.54-3.53 (m, 1H), 3.51-3.43 (m, 1H), 3.25-3.15 (m, 1H), 3.01 (dd, 1H), 2.42-2.39 (m, 1H), 2.32-2.30 (m, 2H), 2.25 (s, 6H), 2.05-1.90 (m, 1H), 1.56-1.52 (m, 1H), 1.18 (dd, 6H). One exchangeable proton not observed.

LCMS: m/z 487.4 (M+H)⁺ (ES⁺).

### Example 154: 4-(5-fluoro-3-isopropyl-2-((5-(phenylthio)-4H-1,2,4-triazol-3-yl)-methyl)phenyl)-2-methoxypyridine

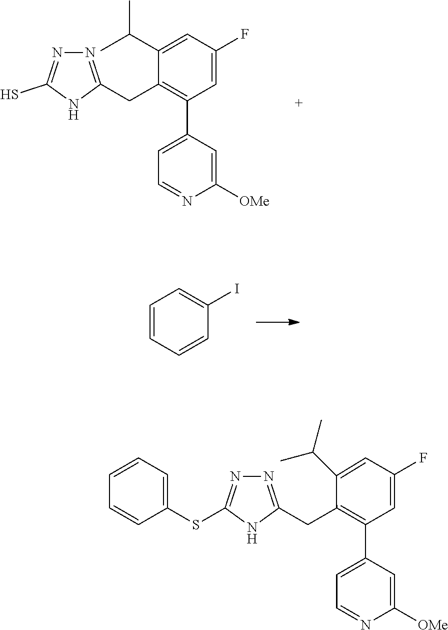 [SMILES: COc1cc(-c2cc(F)cc(C(C)C)c2Cc2nnc(S)[nH]2)ccn1 ; Ic1ccccc1 ; COc1cc(-c2cc(F)cc(C(C)C)c2Cc2nnc(Sc3ccccc3)[nH]2)ccn1]

To a solution of 5-(4-fluoro-2-isopropyl-6-(2-methoxypyridin-4-yl)benzyl)-4H-1,2,4-triazole-3-thiol (Intermediate R15) (300 mg, 836.98 μmol, 1 eq) in dioxane (1 mL) was added iodobenzene (162 mg, 795.13 μmol, 0.95 eq), CuI (318 mg, 1.67 mmol, 2 eq) and N,N′-dimethylethane-1,2-diamine (1.48 g, 16.74 mmol, 20 eq). The mixture was stirred at 70° C. for 0.5 hour under N₂. The reaction mixture was filtered, and the filtrate was concentrated in vacuum. The residue was purified by silica gel column chromatography (SiO₂, petroleum ether:ethyl acetate, 50:1 to 1:1) to give the title compound (180 mg, 49.5% yield) as a yellow solid.

¹H NMR (400 MHz, CDCl₃) δ 8.13 (d, 1H), 7.52-7.49 (m, 2H), 7.40-7.33 (m, 3H), 7.07 (dd, 1H), 6.81-6.78 (m, 2H), 6.69 (s, 1H), 4.02 (s, 2H), 3.94 (s, 3H), 3.13-3.06 (m, 1H), 1.14 (d, 6H). One exchangeable proton not observed.

LCMS: m/z 435.1 (M+H)⁺ (ES⁺).

### Example 155:4-(5-fluoro-3-isopropyl-2-((5-(phenylsulfonyl)-4H-1,2,4-triazol-3-yl)-methyl)phenyl)-2-methoxypyridine

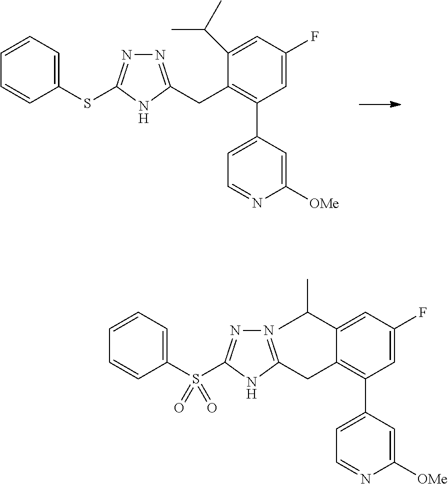 [SMILES: COc1cc(-c2cc(F)cc(C(C)C)c2Cc2nnc(Sc3ccccc3)[nH]2)ccn1 ; COc1cc(-c2cc(F)cc(C(C)C)c2Cc2nnc(S(=O)(=O)c3ccccc3)[nH]2)ccn1]

To a solution of 4-(5-fluoro-3-isopropyl-2-((5-(phenylthio)-4H-1,2,4-triazol-3-yl)-methyl)phenyl)-2-methoxypyridine (Example 154) (180 mg, 414.24 μmol, 1 eq) in MeOH (1.5 mL) and H₂O (1.5 mL) was added Oxone (509 mg, 828.49 μmol, 2 eq). The mixture was stirred at 25° C. for 12 hours. The reaction solution was diluted with H₂O (5 mL) and extracted with EtOAc (3×5 mL). The organic phases were dried over anhydrous Na₂SO₄, filtered and concentrated in vacuum. The residue was dissolved in DCM (2 mL), and then to the above solution was added 4,4,5,5-tetramethyl-2-(4,4,5,5-tetramethyl-1,3,2-dioxaborolan-2-yl)-1,3,2-dioxaborolane (200 mg). The mixture was stirred at 25° C. for 2 hours. The mixture was filtered and the filtrate was concentrated in vacuum. The residue was purified by prep-HPLC (column: Waters Xbridge C18, 150 mm*25 mm*5 μm; mobile phase: [A: water (0.05% ammonia hydroxide v/v), B: MeCN]; B %: 15%-45%, 10 min) to give the title compound (90.43 mg, 46.61% yield, 99.6% purity on LCMS) as a white solid.

¹H NMR (400 MHz, CDCl₃) δ 8.06-8.02 (m, 3H), 7.67-7.63 (m, 1H), 7.56-7.51 (m, 2H), 7.06 (dd, 1H), 6.78 (dd, 1H), 6.64 (dd, 1H), 6.56 (s, 1H), 4.08 (s, 2H), 3.87 (s, 3H), 3.02-2.93 (m, 1H), 1.06 (d, 6H). One exchangeable proton not observed.

LCMS: m/z 467.4 (M+H)⁺ (ES⁺).

### Example 156: 1-(1-((5-(4-fluoro-2-isopropyl-6-(2-methoxypyridin-4-yl)benzyl)-4H-1,2,4-triazol-3-yl)sulfonyl)pyrrolidin-3-yl)-N,N-dimethylmethanamine

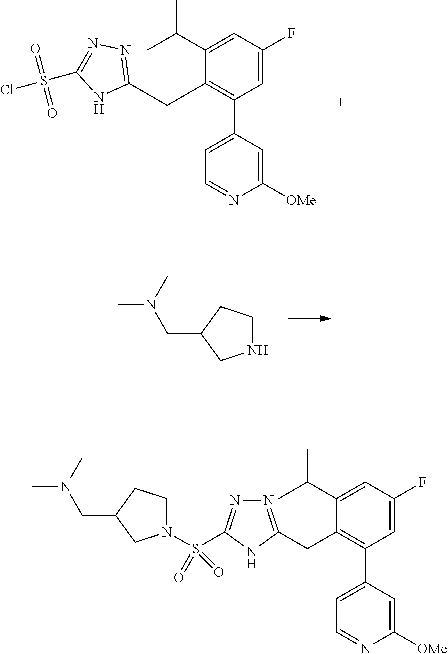

To a solution of N,N-dimethyl-1-(pyrrolidin-3-yl)methanamine (54 mg, 423.65 μmol, 1.5 eq) in DCM (4 mL) was added 5-(4-fluoro-2-isopropyl-6-(2-methoxypyridin-4-yl)-benzyl)-4H-1,2,4-triazole-3-sulfonyl chloride (Intermediate R16) (120 mg, 282.44 μmol, 1 eq) and DIEA (365 mg, 2.82 mmol, 10 eq). The mixture was stirred at 25° C. for 1.5 hours. The reaction mixture was diluted with H₂O (20 mL) and extracted with DCM (3×15 mL). The organic phases were dried over anhydrous Na₂SO₄, filtered and concentrated in vacuum. The residue was purified by prep-HPLC (column: Xtimate C18,150 mm*25 mm*5 μm; mobile phase: [A: water (0.05% ammonia hydroxide v/v), B: MeCN]; B %: 20%-50%, 10 min) to give the title compound (15.07 mg, yield over 2 steps: 5.2%, 100% purity on LCMS) as a white solid.

¹H NMR (400 MHz, CDCl₃) δ 8.14 (d, 1H), 7.07 (dd, 1H), 6.81-6.78 (m, 2H), 6.70 (s, 1H), 4.09 (s, 2H), 3.92 (s, 3H), 3.55-3.65 (m, 2H), 3.48-3.41 (m, 1H), 3.13-3.02 (m, 2H), 2.42-2.18 (m, 3H), 2.32 (s, 6H), 2.10-1.97, (m, 1H), 1.61-1.55 (m, 1H), 1.15-1.13 (m, 6H). One exchangeable proton not observed.

LCMS: m/z 517.4 (M+H)⁺ (ES⁺).

### Example 157: N-(1,2,3,5,6,7-hexahydro-s-indacen-4-yl)-5-((tetrahydro-2H-pyran-4-yl)sulfonyl)-4H-1,2,4-triazol-3-amine

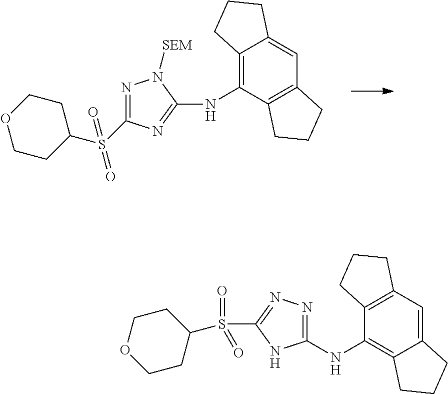 [SMILES: C[Si](C)(C)CCOCn1nc(S(=O)(=O)C2CCOCC2)nc1Nc1c2c(cc3c1CCC3)CCC2 ; O=S(=O)(c1nnc(Nc2c3c(cc4c2CCC4)CCC3)[nH]1)C1CCOCC1]

To a solution of N-(1,2,3,5,6,7-hexahydro-s-indacen-4-yl)-3-((tetrahydro-2H-pyran-4-yl)sulfonyl)-1-((2-(trimethylsilyl)ethoxy)methyl)-1H-1,2,4-triazol-5-amine (Intermediate B10) (110 mg, 212.05 μmol, 1 eq) in THF (1 mL) was added TBAF (1 M, 848.20 μL, 4 eq). The mixture was stirred at 70° C. for 24 hours. The reaction mixture was diluted with DCM (10 mL) and washed with 1 N aqueous HCl solution (3×5 mL). The organic layers were dried over anhydrous Na₂SO₄, filtered and concentrated in vacuum. The residue was purified by prep-HPLC (column: Phenomenex Synergi C18, 150 mm*25 mm*10 μm; mobile phase: [A: water (0.1% TFA v/v), B: MeCN]; B %: 40%-65%, 9 minutes) to give the title compound (65.03 mg, 78.94% yield, 100% purity on LCMS) as a white solid.

¹H NMR (400 MHz, CD₃OD) δ 7.02 (s, 1H), 4.03 (dd, 2H), 3.61-3.53 (m, 1H), 3.46-3.39 (m, 2H), 2.90 (t, 4H), 2.73 (t, 4H), 2.11-2.03 (m, 4H), 1.96-1.78 (m, 4H). Two exchangeable protons not observed.

LCMS: m/z 389.1 (M+H)⁺ (ES⁺).

### Example 158: 1-(1-((5-((1,2,3,5,6,7-hexahydro-s-indacen-4-yl)methyl)-4H-1,2,4-triazol-3-yl)sulfonyl)pyrrolidin-3-yl)-N,N-dimethylmethanamine

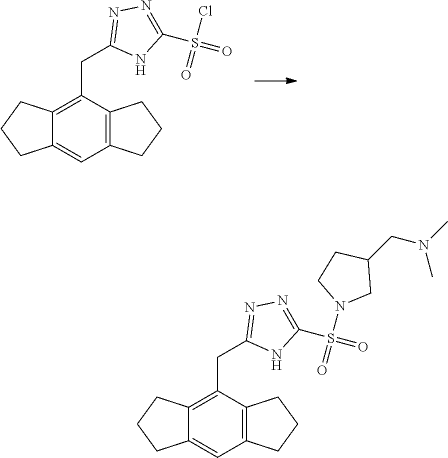 [SMILES: O=S(=O)(Cl)c1nnc(Cc2c3c(cc4c2CCC4)CCC3)[nH]1 ; CN(C)CC1CCN(S(=O)(=O)c2nnc(Cc3c4c(cc5c3CCC5)CCC4)[nH]2)C1]

To a solution of 5-((1,2,3,5,6,7-hexahydro-s-indacen-4-yl)methyl)-4H-1,2,4-triazole-3-sulfonyl chloride (Intermediate R17) (100 mg, 296.01 μmol, 1 eq) in DCM (5 mL) was added DIPEA (115 mg, 888.04 μmol, 3 eq) and N,N-dimethyl-1-(pyrrolidin-3-yl)-methanamine (38 mg, 296.01 μmol, 1 eq). The mixture was stirred at 25° C. for 12 hours. The reaction mixture was diluted with DCM (10 mL), washed with water (3×5 mL) and brine (3×5 mL). The organic layer was dried over anhydrous Na₂SO₄, filtered and concentrated in vacuum. The residue was purified by prep-HPLC (column: Luna C18, 150 mm*25 mm*5 μm; mobile phase: [A: water (0.075% TFA v/v), B: MeCN]; B %: 18%-48%, 9 minutes) to give the title compound (13.42 mg, yield over two steps: 6.7%,100% purity on LCMS, TFA salt) as a white solid.

¹H NMR (400 MHz, CD₃OD) δ 7.02 (s, 1H), 4.14 (s, 2H), 3.75-3.71 (m, 1H), 3.60-3.52 (m, 1H), 3.45-3.36 (m, 1H), 3.21-3.11 (m, 3H), 2.89-2.83 (m, 10H), 2.76 (t, 4H), 2.65-2.54 (m, 1H), 2.14-1.99 (m, 5H), 1.68-1.58 (m, 1H). One exchangeable proton not observed.

LCMS: m/z 430.5 (M+H)⁺ (ES⁺)

### Example 159: N-(1,2,3,5,6,7-hexahydro-s-indacen-4-yl)-5-((1-methyl-1H-pyrazol-4-yl)sulfonyl)-4H-1,2,4-triazol-3-amine

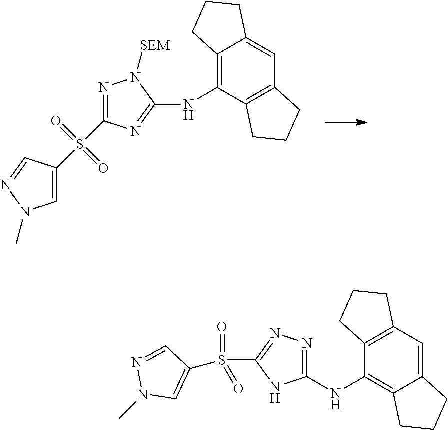 [SMILES: Cn1cc(S(=O)(=O)c2nc(Nc3c4c(cc5c3CCC5)CCC4)n(COCC[Si](C)(C)C)n2)cn1 ; Cn1cc(S(=O)(=O)c2nnc(Nc3c4c(cc5c3CCC5)CCC4)[nH]2)cn1]

To a solution of N-(1,2,3,5,6,7-hexahydro-s-indacen-4-yl)-3-((1-methyl-1H-pyrazol-4-yl)sulfonyl)-1-((2-(trimethylsilyl)ethoxy)methyl)-1H-1,2,4-triazol-5-amine (Intermediate B12) (150 mg, 291.42 μmol, 1 eq) in THF (1 mL) was added TBAF (1 M, 1.17 mL, 4 eq). The mixture was stirred at 70° C. for 24 hours. The reaction mixture was diluted with DCM (10 mL) and washed with 1 N aqueous HCl solution (3×5 mL). The organic layers were dried over anhydrous Na₂SO₄, filtered and concentrated in vacuum. The residue was purified by prep-HPLC (column: Phenomenex Synergi C18, 150 mm*25 mm*10 μm; mobile phase: [A: water (0.1% TFA v/v), B: MeCN]; B %: 38%-58%,10 minutes) to give the title compound (21.7 mg, 19.17% yield, 99% purity on LCMS) as a white solid.

¹H NMR (400 MHz, DMSO-d₆) δ 13.01 (br s, 1H), 8.94 (s, 1H), 8.47 (s, 1H), 7.86 (s, 1H), 6.95 (s, 1H), 3.90 (s, 3H), 2.81 (t, 4H), 2.57 (t, 4H), 1.98-1.91 (m, 4H).

LCMS: m/z 385.1 (M+H)⁺ (ES⁺).

### Example 160: 1-(1-((5-((5-(2-methoxypyridin-4-yl)-2,3-dihydro-1H-inden-4-yl)-methyl)-4H-1,2,4-triazol-3-yl)sulfonyl)pyrrolidin-3-yl)-N,N-dimethylmethanamine

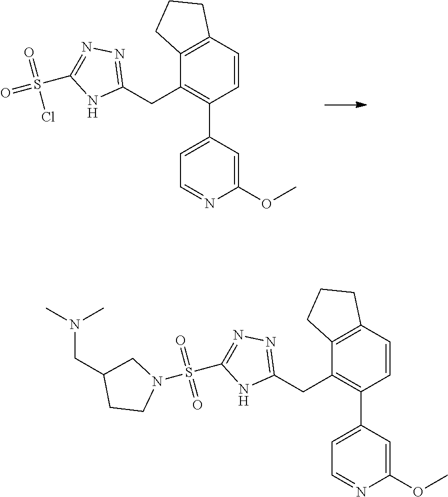

To a solution of 5-((5-(2-methoxypyridin-4-yl)-2,3-dihydro-1H-inden-4-yl)methyl)-4H-1,2,4-triazole-3-sulfonyl chloride (190 mg, 469.29 μmol, 1 eq) in DCM (4 mL) was added DIPEA (303 mg, 2.35 mmol, 5 eq) and N,N-dimethyl-1-(pyrrolidin-3-yl)methanamine (66 mg, 516.22 μmol, 1.1 eq). The mixture was stirred at 20° C. for 2 hours. The reaction mixture was poured into water (20 mL) and extracted with DCM (2×20 mL). The combined organic phases were washed with brine (2×20 mL), dried over anhydrous Na₂SO₄, filtered and concentrated in vacuum. The residue was purified by prep-HPLC (column: Luna C18, 150 mm*25 mm*5 μm; mobile phase: [A: water (0.075% TFA v/v), B: MeCN]; B %: 18%-48%, 9 min) to give title compound (57.62 mg, yield over two steps: 20.0%, 97.8% purity on HPLC, TFA salt) as a white solid.

¹H NMR (400 MHz, CD₃OD) δ 8.13 (d, 1H), 7.31 (d, 1H), 7.10 (d, 1H), 6.94-6.92 (m, 1H), 6.84-6.82 (m, 1H), 4.15 (s, 2H), 3.94 (s, 3H), 3.73-3.69 (m, 1H), 3.58-3.52 (m, 1H), 3.41-3.36 (m, 1H), 3.19-3.14 (m, 3H), 3.01 (t, 2H), 2.88 (s, 6H), 2.82 (t, 2H), 2.69-2.61 (m, 1H), 2.18-2.08 (m, 3H), 1.69-1.63 (m, 1H). One exchangeable proton not observed, TFA proton not observed.

LCMS: m/z 497.3 (M+H)⁺ (ES⁺).

### Example 161: 1-(1-((5-(4-fluoro-2,6-diisopropylbenzyl)-4H-1,2,4-triazol-3-yl)-sulfonyl)pyrrolidin-3-yl)-N,N-dimethylmethanamine

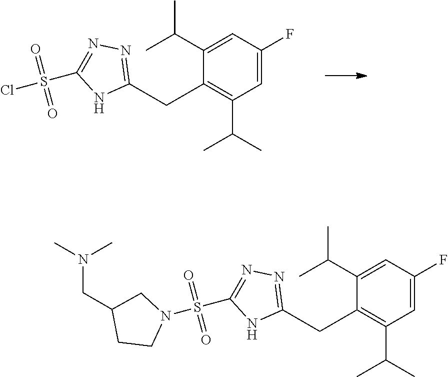 [SMILES: CC(C)c1cc(F)cc(C(C)C)c1Cc1nnc(S(=O)(=O)Cl)[nH]1 ; CC(C)c1cc(F)cc(C(C)C)c1Cc1nnc(S(=O)(=O)N2CCC(CN(C)C)C2)[nH]1]

To a mixture of 5-(4-fluoro-2,6-diisopropylbenzyl)-4H-1,2,4-triazole-3-sulfonyl chloride (Intermediate R19) (0.25 g, 694.74 μmol, 1 eq) in DCM (4 mL) were added DIPEA (269 mg, 2.08 mmol, 3 eq) and N,N-dimethyl-1-pyrrolidin-3-ylmethanamine (134 mg, 1.04 mmol, 1.5 eq) in one portion. Then the reaction mixture was stirred at 20° C. for 12 hours. The reaction solution was concentrated in vacuum. The crude product was purified by prep-HPLC (column: Luna C18, 150 mm*25 mm*5 μm; mobile phase: [A: water (0.075% TFA v/v), MeCN]; B %: 22%-52%, 9 min) to give the title compound (12.76 mg, yield over two steps: 4.1%, 100% purity on LCMS) as a white solid.

¹H NMR (400 MHz, CD₃OD) δ 6.94 (d, 2H), 4.32 (s, 2H), 3.71-3.70 (m, 1H), 3.68-3.55 (m, 1H), 3.40-3.38 (m, 1H), 3.18-3.13 (m, 5H), 2.88 (s, 6H), 2.65-2.62 (m, 1H), 2.10-2.07 (m, 1H), 1.66-1.60 (m, 1H), 1.17 (d, 12H). One exchangeable proton not observed.

LCMS: m/z 452.4 (M+H)⁺ (ES⁺).

### Example 162: N-(1,2,3,5,6,7-hexahydro-s-indacen-4-yl)-3-(pyridazin-3-ylthio)-1H-1,2,4-triazol-5-amine

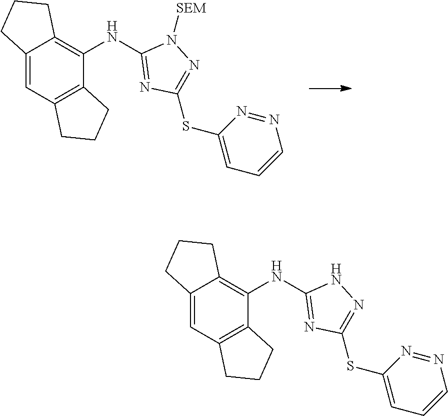 [SMILES: C[Si](C)(C)CCOCn1nc(Sc2cccnn2)nc1Nc1c2c(cc3c1CCC3)CCC2 ; c1cnnc(Sc2n[nH]c(Nc3c4c(cc5c3CCC5)CCC4)n2)c1]

To a solution of N-(1,2,3,5,6,7-hexahydro-s-indacen-4-yl)-3-(pyridazin-3-ylthio)-1-((2-(trimethylsilyl)ethoxy)methyl)-1H-1,2,4-triazol-5-amine (Intermediate B13) (210 mg, 436.86 μmol, 1 eq) in THF (10 mL) was added TBAF (1 M in THF, 1.75 mL, 4 eq). The mixture was stirred at 70° C. for 16 hours. The reaction solution was diluted with water (10 mL) and extracted with EtOAc (2×10 mL). The organic layers were dried over anhydrous Na₂SO₄, filtered and concentrated in vacuum to give the title compound (150 mg, crude) as a brown solid.

¹H NMR (400 MHz, DMSO-d₆) δ 12.80 (s, 1H), 9.04 (d, 1H), 8.77 (s, 1H), 7.64-7.61 (m, 1H), 7.51 (d, 1H), 6.92 (s, 1H), 2.78 (t, 4H), 2.64 (t, 4H), 1.98-1.95 (m, 4H).

LCMS: m/z 351.1 (M+H)⁺ (ES⁺).

The following examples were synthesised following the general procedure for Example 162, from the intermediate compounds indicated in the ‘From’ column:

| Example | Structure | ¹H NMR spectrum | LCMS | From |
|---|---|---|---|---|
| 163 | 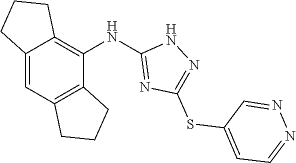 [SMILES: c1cc(Sc2n[nH]c(Nc3c4c(cc5c3CCC5)CCC4)n2)cnn1] N-(1,2,3,5,6,7-hexahydro-s- indacen-4-yl)-3-(pyridazin-4- ylthio)-1H-1,2,4-triazol-5-amine | ¹H NMR (400 MHz, DMSO-d₆) δ 12.86 (s, 1H), 9.13 (d, 1H), 9.02 (d, 1H), 8.82 (s, 1H), 7.58 (dd, 1H), 6.94 (s, 1H), 2.81 (t, 4H), 2.65 (t, 4H), 1.99-1.96 (m, 4H). | m/z 351.1 (M + H)⁺ (ES⁺); | B33 |
| 164 | 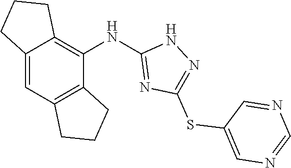 [SMILES: c1ncc(Sc2n[nH]c(Nc3c4c(cc5c3CCC5)CCC4)n2)cn1] N-(1,2,3,5,6,7-hexahydro-s- indacen-4-yl)-3-(pyrimidin-5- ylthio)-1H-1,2,4-triazol-5-amine | ¹H NMR (400 MHz, DMSO-d₆) δ 12.53 (s, 1H), 9.11 (s, 1H), 8.88 (s, 2H), 8.69 (s, 1H), 6.92 (d, 1H), 2.80 (t, 4H), 2.60 (t, 4H), 1.97-1.93 (m, 4H). | m/z 351.1 (M + H)⁺ (ES⁺); | B32 |
| 165 | 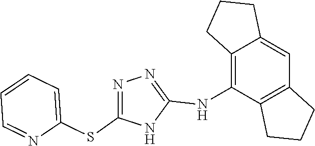 [SMILES: c1ccc(Sc2nnc(Nc3c4c(cc5c3CCC5)CCC4)[nH]2)nc1] N-(1,2,3,5,6,7-hexahydro-s- indacen-4-yl)-5-(pyridin-2- ylthio)-4H-1,2,4-triazol-3-amine | ¹H NMR (400 MHz, CDCl₃) δ 8.50 (d, 1H, 7.65 (t, 1H), 7.37 (d, 1H), 7.19-7.15 (m, 1H), 6.97 (s, 1H), 2.90 (t, 4H), 2.80 (t, 4H), 2.11-2.03 (m, 4H). Two exchangeable protons not observed. | m/z 349.9 (M + H)⁺ (ES⁺); | B20 |
| 166 | 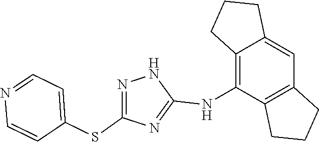 [SMILES: c1cc(Sc2n[nH]c(Nc3c4c(cc5c3CCC5)CCC4)n2)ccn1] N-(1,2,3,5,6,7-hexahydro-s- indacen-4-yl)-3-(pyridin-4- ylthio)-1H-1,2,4-triazol-5-amine | ¹H NMR (400 MHz, DMSO-d₆) δ 12.79 (s, 1H), 8.75 (s, 1H), 8.42-8.40 (m, 2H), 7.25 (d, 2H), 6.93 (s, 1H), 2.81 (t, 4H), 2.65 (t, 4H), 1.96-1.94 (m, 4H). | m/z 350.2 (M + H)⁺ (ES⁺); | B26 |

### Example 167: N-(1,2,3,5,6,7-hexahydro-s-indacen-4-yl)-3-(pyridazin-3-ylsulfonyl)-1H-1,2,4-triazol-5-amine

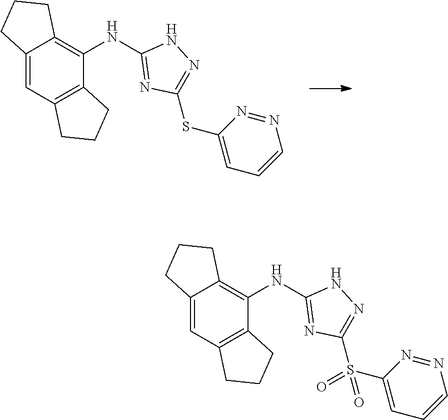 [SMILES: c1cnnc(Sc2n[nH]c(Nc3c4c(cc5c3CCC5)CCC4)n2)c1 ; O=S(=O)(c1cccnn1)c1n[nH]c(Nc2c3c(cc4c2CCC4)CCC3)n1]

A solution of ammonium molybdate (20 mg, 99.8 μmol, 0.25 eq) in H₂O₂ (1.8 g, 15.98 mmol, 30% purity in H₂O, 40 eq) was prepared at ° C. To a solution of N-(1,2,3,5,6,7-hexahydro-s-indacen-4-yl)-3-(pyridazin-3-ylthio)-1H-1,2,4-triazol-5-amine (Example 162) (140 mg, 399.50 μmol, 1 eq) in EtOH (5 mL) was added dropwise the above solution at 0° C. After addition, the mixture was heated to 50° C. and stirred for 2 hours. Then the reaction was stirred at 30° C. for 16 hours. The reaction was quenched with saturated aqueous Na₂SO₃ solution (10 mL). The mixture was stirred for 10 minutes, and then concentrated in vacuum to remove EtOH. The residue was diluted with water (5 mL) and extracted with EtOAc (2×10 mL). The organic layers were dried over Na₂SO₄, filtered and concentrated in vacuum. The residue was dissolved in MeOH (2 mL). To the above solution was added 4,4,4′,4′,5,5,5′,5′-octamethyl-2,2′-bi(1,3,2-dioxaborolane) (30 mg). The mixture was stirred at 20° C. for 1 hour and then concentrated in vacuum. The residue was purified by prep-HPLC (column: Phenomenex Synergi C18, 150 mm*25 mm*10 m; mobile phase: [A: water (0.1% TFA v/v); B: MeCN]; B %: 35%-55%, 10 min) to give the title compound (15.82 mg, yield: 10.25% yield, 99% purity on LCMS) as a yellow solid.

¹H NMR (400 MHz, CD₃OD) δ 9.43 (dd, 1H), 8.45 (dd, 1H), 8.03 (dd, 1H), 6.98 (s, 1H), 2.86 (t, 4H), 2.65 (t, 4H), 2.06-2.01 (m, 4H). Two exchangeable protons not observed.

LCMS: m/z 383.0 (M+H)⁺ (ES⁺).

The following examples were synthesised following the general procedure for Example 167, starting from Examples 163 to 166 respectively:

| Example | Structure | ¹H NMR spectrum | LCMS |
|---|---|---|---|
| 168 | 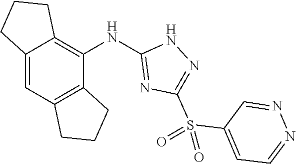 [SMILES: O=S(=O)(c1ccnnc1)c1n[nH]c(Nc2c3c(cc4c2CCC4)CCC3)n1] N-(1,2,3,5,6,7-hexahydro-s-indacen-4- yl)-3-(pyridazin-4-ylsulfonyl)-1H- 1,2,4-triazol-5-amine | ¹H NMR (400 MHz, CD₃OD) δ 9.68 (d, 1H), 9.58 (d, 1H), 8.23 (dd, 1H), 7.01 (s, 1H), 2.88 (t, 4H), 2.64 (t, 4H), 2.07-1.99 (m, 4H). Two exchangeable protons not observed. | m/z 383.1 (M + H)⁺ (ES⁺) |
| 169 | 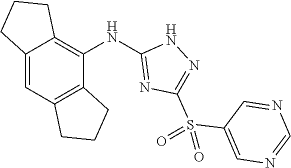 [SMILES: O=S(=O)(c1cncnc1)c1n[nH]c(Nc2c3c(cc4c2CCC4)CCC3)n1] N-(1,2,3,5,6,7-hexahydro-s-indacen-4- yl)-3-(pyrimidin-5-ylsulfonyl)-1H- 1,2,4-triazol-5-amine | ¹H NMR (400 MHz, CD₃OD) δ 9.44 (s, 1H), 9.32 (s, 2H), 7.00 (s, 1H), 2.88 (t, 4H), 2.65 (t, 4H), 2.06-2.01 (m, 4H). Two exchangeable protons not observed. | m/z 383.1 (M + H)⁺ (ES⁺) |
| 170 | 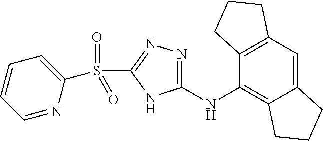 [SMILES: O=S(=O)(c1ccccn1)c1nnc(Nc2c3c(cc4c2CCC4)CCC3)[nH]1] N-(1,2,3,5,6,7-hexahydro-s-indacen-4- yl)-5-(pyridin-2-ylsulfonyl)-4H-1,2,4- triazol-5-amine | ¹H NMR (400 MHz, CD₃OD) δ 8.70 (d, 1H), 8.23 (dd, 1H), 8.15-8.10 (m, 1H), 7.70-7.68 (m, 1H), 6.97 (s, 1H), 2.86 (t, 4H), 2.64 (t, 4H), 2.05-1.99 (m, 4H). Two exchangeable protons not observed. | m/z 381.9 (M + H)⁺ (ES⁺) |
| 171 | 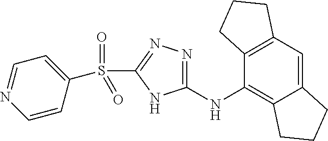 [SMILES: O=S(=O)(c1ccncc1)c1nnc(Nc2c3c(cc4c2CCC4)CCC3)[nH]1] N-(1,2,3,5,6,7-hexahydro-s-indacen-4- yl)-5-(pyridin-4-ylsulfonyl)-4H-1,2,4- triazol-3-amine | ¹H NMR (400 MHz, DMSO-d₆) δ 13.31 (s, 1H), 9.05 (s, 1H), 8.93 (d, 2H), 7.86 (d, 2H), 6.95 (s, 1H), 2.80 (t, 4H), 2.52 (t, 4H), 1.92 (m, 4H). | m/z 382.2 (M + H)⁺ (ES⁺) |

### Example 172: 2-(3-((5-((1,2,3,5,6,7-hexahydro-s-indacen-4-yl)amino)-4H-1,2,4-triazol-3-yl)sulfonyl)phenyl)propan-2-ol

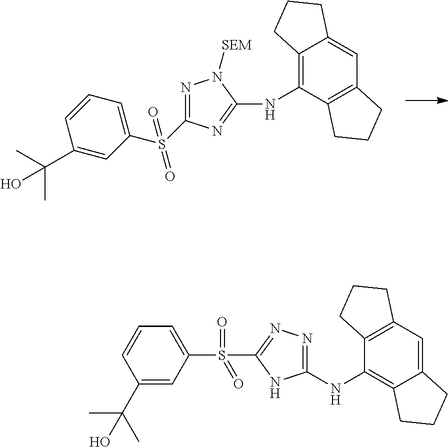 [SMILES: CC(C)(O)c1cccc(S(=O)(=O)c2nc(Nc3c4c(cc5c3CCC5)CCC4)n(COCC[Si](C)(C)C)n2)c1 ; CC(C)(O)c1cccc(S(=O)(=O)c2nnc(Nc3c4c(cc5c3CCC5)CCC4)[nH]2)c1]

To a solution of 2-(3-((5-((1,2,3,5,6,7-hexahydro-s-indacen-4-yl)amino)-1-((2-(trimethylsilyl)ethoxy)methyl)-1H-1,2,4-triazol-3-yl)sulfonyl)phenyl)propan-2-ol (Intermediate B35) (60 mg, 105.49 μmol, 1 eq) in THF (0.5 mL) was added TBAF (1 M, 421.94 μL, 4 eq). The mixture was stirred at 70° C. for 12 hours. The reaction mixture was diluted with DCM (10 mL), washed with 1 N aqueous HCl solution (3×5 mL), then washed with saturated aqueous NaHCO₃ solution (3×5 mL). The organic layer was dried over anhydrous Na₂SO₄, filtered and concentrated in vacuum. The residue was purified by prep-HPLC (column: Luna C18,150 mm*25 mm*5 μm; mobile phase: [A: water (0.075% TFA v/v), B: MeCN]; B %: 38%-68%, 2 minutes) to give the title compound (12 mg, 25.4% yield, 98% purity on LCMS) as a white solid.

¹H NMR (400 MHz, CD₃OD) δ 8.17 (t, 1H), 7.90-7.82 (m, 2H), 7.57 (t, 1H), 6.98 (s, 1H), 2.86 (t, 4H), 2.62 (t, 4H), 2.05-197 (m, 4H), 1.54 (s, 6H). Three exchangeable protons not observed.

LCMS: m/z 439.2 (M+H)⁺ (ES⁺).

The following examples were synthesised following the general procedure for Example 172, from the intermediate compounds indicated in the ‘From’ column:

| Example | Structure | ¹H NMR spectrum | LCMS | From |
|---|---|---|---|---|
| 173 | 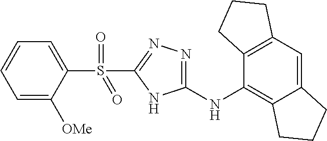 [SMILES: COc1ccccc1S(=O)(=O)c1nnc(Nc2c3c(cc4c2CCC4)CCC3)[nH]1] N-(1,2,3,5,6,7-hexahydro-s- indacen-4-yl)-5-((2- methoxyphenyl)sulfonyl)-4H- 1,2,4-triazol-3-amine | ¹H NMR (400 MHz, CDCl₃) δ 8.15 (dd, 1H), 8.01 (s, 1H), 7.67-7.62 (m, 1H), 7.16 (t, 1H), 6.97 (d, 1H), 6.90 (s, 1H), 3.70 (s, 3H), 2.82-2.76 (m, 4H), 2.54-2.50 (m, 4H), 1.91-1.84 (m, 4H). One exchangeable proton not observed. | m/z 410.9 (M + H)⁺ (ES⁺) | B37 |
| 174 | 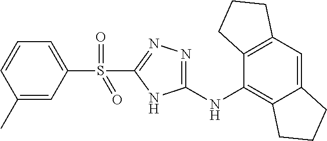 [SMILES: Cc1cccc(S(=O)(=O)c2nnc(Nc3c4c(cc5c3CCC5)CCC4)[nH]2)c1] N-(1,2,3,5,6,7-hexahydro-s- indacen-4-yl)-5-(m- tolylsulfonyl)-4H-1,2,4-triazol- 3-amine | ¹H NMR (400 MHz, CDCl₃) δ 8.05 (br s, 1H), 7.90-7.85 (m, 2H), 7.51-7.43 (m, 2H), 7.02 (s, 1H), 2.87 (t, 4H), 2.67 (t, 4H), 2.44 (s, 3H), 2.05- 1.98 (m, 4H). One exchangeable proton not observed. | m/z 395.2 (M + H)⁺ (ES⁺) | B41 |
| 175 | 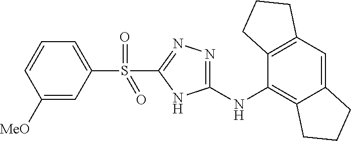 [SMILES: COc1cccc(S(=O)(=O)c2nnc(Nc3c4c(cc5c3CCC5)CCC4)[nH]2)c1] N-(1,2,3,5,6,7-hexahydro-s- indacen-4-yl)-5-((3- methoxyphenyl)sulfonyl)-4H- 1,2,4-triazol-3-amine | ¹H NMR (400 MHz, CDCl₃) δ 8.09 (s, 1H), 7.63 (d, 1H), 7.57 (d, 1H), 7.46 (t, 1H), 7.22 (dd, 1H), 7.02 (s, 1H), 3.85 (s, 3H), 2.88 (t, 4H), 2.68 (t, 4H), 2.06-1.97 (m, 4H). One exchangeable proton not observed. | m/z 410.9 (M + H)⁺ (ES⁺) | B42 |
| 176 | 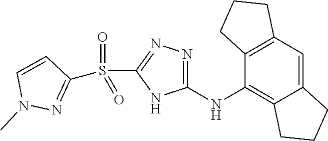 [SMILES: Cn1ccc(S(=O)(=O)c2nnc(Nc3c4c(cc5c3CCC5)CCC4)[nH]2)n1] N-(1,2,3,5,6,7-hexahydro-s- indacen-4-yl)-5-((1-methyl- 1H-pyrazol-3-yl)sulfonyl)-4H- 1,2,4-triazol-3-amine | ¹H NMR (400 MHz, CDCl₃) δ 7.93 (s, 1H), 7.44 (d, 1H), 6.98 (s, 1H), 6.94 (d, 1H), 3.86 (s, 3H), 2.87 (t, 4H), 2.72 (t, 4H), 2.05-1.97 (m, 4H). One exchangeable proton not observed. | m/z 385.2 (M + H)⁺ (ES⁺) | B44 |
| 177 | 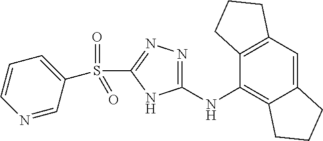 [SMILES: O=S(=O)(c1cccnc1)c1nnc(Nc2c3c(cc4c2CCC4)CCC3)[nH]1] N-(1,2,3,5,6,7-hexahydro-s- indacen-4-yl)-5-(pyridin-3- ylsulfonyl)-4H-1,2,4-triazol-3- amine | ¹H NMR (400 MHz, CD₃OD) δ 9.13 (d, 1H), 8.87-8.85 (m, 1H), 8.42-8.39 (m, 1H), 7.67 (dd, 1H), 6.99 (s, 1H), 2.87 (t, 4H), 2.63 (t, 4H), 2.05-1.98 (t, 4H). Two exchangeable protons not observed. | m/z 381.9 (M + H)⁺ (ES⁺) | B45 |
| 178 | 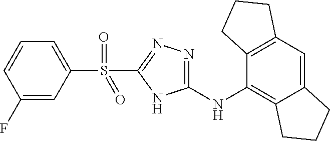 [SMILES: O=S(=O)(c1cccc(F)c1)c1nnc(Nc2c3c(cc4c2CCC4)CCC3)[nH]1] 5-((3-fluorophenyl)sulfonyl)- N-(1,2,3,5,6,7-hexahydro-s- indacen-4-yl)-4H-1,2,4-triazol- 3-amine | ¹H NMR (400 MHz, CDCl₃) δ 7.87 (d, 1H), 7.78 (dd, 1H), 7.67 (s, 1H), 7.61-7.55 (m, 1H), 7.39-7.37 (m, 1H), 7.01 (s, 1H), 2.88 (t, 4H), 2.67 (t, 4H), 2.05-1.97 (m, 4H). One exchangeable proton not observed. | m/z 399.1 (M + H)⁺ (ES⁺) | B46 |
| 179 | 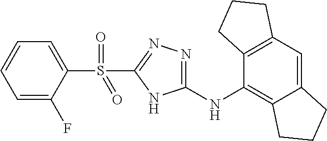 [SMILES: O=S(=O)(c1nnc(Nc2c3c(cc4c2CCC4)CCC3)[nH]1)c1ccccc1F] 5-((2-fluorophenyl)sulfonyl)- N-(1,2,3,5,6,7-hexahydro-s- indacen-4-yl)-4H-1,2,4-triazol- 3-amine | ¹H NMR (400 MHz, CDCl₃) δ. 8.12 (t, 1H), 8.00 (s, 1H), 7.73- 7.69 (m, 1H), 7.38 (t, 1H), 7.21 (t, 1H), 6.97 (s, 1H), 2.83 (t, 4H), 2.7 (t, 4H), 2.01-1.93 (m, 4H). One exchangeable proton not observed. | m/z 399.1 (M + H)⁺ (ES⁺) | B48 |

### Example 180: 6-((5-((1,2,3,5,6,7-hexahydro-s-indacen-4-yl)amino)-1H-1,2,4-triazol-3-yl)sulfonyl)-2-methylisoindolin-1-one

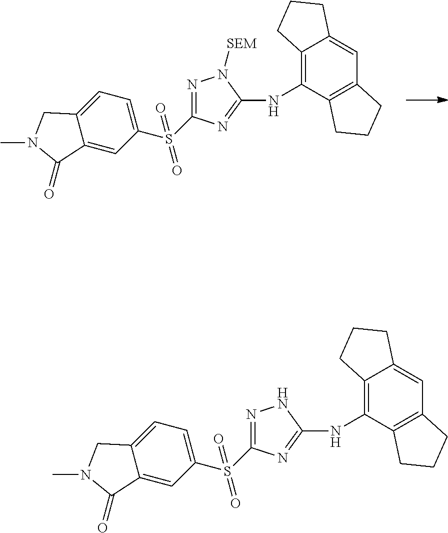 [SMILES: CN1Cc2ccc(S(=O)(=O)c3nc(Nc4c5c(cc6c4CCC6)CCC5)n(COCC[Si](C)(C)C)n3)cc2C1=O ; CN1Cc2ccc(S(=O)(=O)c3n[nH]c(Nc4c5c(cc6c4CCC6)CCC5)n3)cc2C1=O]

To a solution of 6-((5-(1,2,3,5,6,7-hexahydro-s-indacen-4-yl)amino)-1-((2-(trimethylsilyl)ethoxy)methyl)-1H-1,2,4-triazol-3-yl) sulfonyl)-2-methylisoindolin-1-one (Intermediate B36) (20 mg, 34.50 μmol, 1 eq) in MeOH (1 mL) was added HCl/dioxane (4 M, 8.62 μmol, 1 eq). The mixture was stirred at 70° C. for 1 hour. The mixture was concentrated in vacuum. The residue was purified by prep-HPLC (column: Luna C18, 150 mm*25 mm*5 μm; mobile phase: [A: water (0.1% TFA v/v), B: MeCN]; B %: 30%-60%, 11 min) to give the title compound (4.44 mg, 28.67% yield, 100% purity on LCMS) as a white solid.

¹H NMR (400 MHz, DMSO-d₆) δ 13.18 (s, 1H), 9.00 (s, 1H), 8.12 (dd, 1H), 8.06 (d, 1H), 7.87 (dd, 1H), 6.93 (s, 1H), 4.59 (s, 2H), 3.09 (s, 3H), 2.77 (t, 4H), 2.52-2.51 (m, 4H), 1.93-1.86 (m, 4H).

LCMS: m/z 450.0 (M+H)⁺ (ES⁺)

The following examples were synthesised following the general procedure for Example 180, from the intermediate compounds indicated in the ‘From’ column:

| | | ¹H NMR | | |
|---|---|---|---|---|
| Example | Structure | spectrum | LCMS | From |
| 181 | 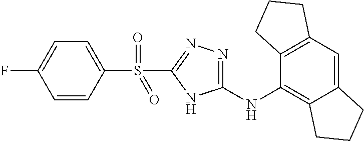 [SMILES: O=S(=O)(c1ccc(F)cc1)c1nnc(Nc2c3c(cc4c2CCC4)CCC3)[nH]1] | ¹H NMR (400 MHz, DMSO- d₆) δ 7.96 (dd, 2H), 7.48 (t, 2H), 6.92 (s, 1H), 2.76 (t, 4H), 2.44 (t, 4H), 1.92-1.84 (m, 4H). Two | m/z 399.0 (M + H)⁺ (ES⁺) | B40 |
| | 5-((4-fluorophenyl)sulfonyl)-N- | exchangeable | | |
| | (1,2,3,5,6,7-hexahydro-s-indacen-4-yl)- | protons not | | |
| | 4H-1,2,4-triazol-3-amine | observed. | | |
| 182 | 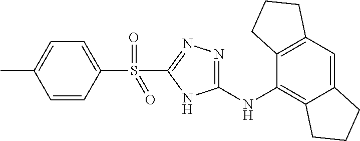 [SMILES: Cc1ccc(S(=O)(=O)c2nnc(Nc3c4c(cc5c3CCC5)CCC4)[nH]2)cc1] | ¹H NMR (400 MHz, CDCl₃): δ 7.96 (d, 2H), 7.93 (s, 1H), 7.37 (d, 2H), 7.03 (s, 1H), 2.88 (t, 4H), 2.67 (t, 4H), 2.46 (s, | m/z 395.2 (M + H)⁺ (ES⁺) | B43 |
| | N-(1,2,3,5,6,7-hexahydro-s-indacen-4- | 3H), 2.05- | | |
| | yl)-5-tosyl-4H-1,2,4-triazol-3-amine | 1.98 (m, 4H). | | |
| | | One | | |
| | | exchangeable | | |
| | | proton not | | |
| | | observed. | | |
| 183 | 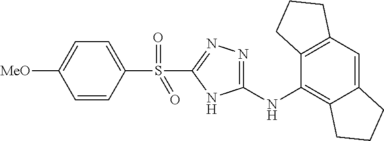 [SMILES: COc1ccc(S(=O)(=O)c2nnc(Nc3c4c(cc5c3CCC5)CCC4)[nH]2)cc1] | ¹H NMR (400 MHz, CDCl₃): δ 8.01 (d, 2H), 7.43 (s, 1H), 7.02 (s, 1H), 7.00 (d, 2H), 3.89 (s, 3H), 2.87 (t, 4H), 2.67 (t, | m/z 411.2 (M + H)⁺ (ES⁺) | B49 |
| | N-(1,2,3,5,6,7-hexahydro-s-indacen-4- | 4H), 2.02- | | |
| | yl)-5-((4-methoxyphenyl)sulfonyl)-4H- | 1.98 (m, 4H). | | |
| | 1,2,4-triazol-3-amine | One | | |
| | | exchangeable | | |
| | | proton not | | |
| | | observed. | | |
| 184 | 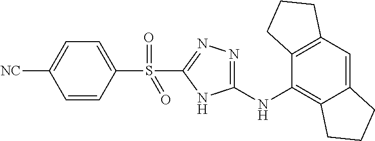 [SMILES: N#Cc1ccc(S(=O)(=O)c2nnc(Nc3c4c(cc5c3CCC5)CCC4)[nH]2)cc1] | ¹H NMR (400 MHz, CDCl₃) δ 8.22 (d, 2H), 7.86 (d, 2H), 7.08 (s, 1H), 6.81 (s, 1H), 2.91 (t, 4H), 2.68 (t, 4H), 2.09- | m/z 406.2 (M + H)⁺ (ES⁺) | B50 |
| | 4-((5-((1,2,3,5,6,7-hexahydro-s- | 2.02 (m, 4H). | | |
| | indacen-4-yl)amino)-4H-1,2,4-triazol- | One | | |
| | 3-yl)sulfonyl)benzonitrile | exchangeable | | |
| | | proton not | | |
| | | observed. | | |
| 185 | 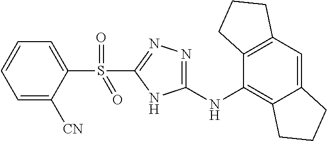 [SMILES: N#Cc1ccccc1S(=O)(=O)c1nnc(Nc2c3c(cc4c2CCC4)CCC3)[nH]1] | ¹H NMR (400 MHz, CDCl₃) δ 8.37 (dd, 1H), 7.90-7.77 (m, 3H), 7.54 (s, 1H), 7.00 (s, 1H), 2.87- 2.84 (t, 4H), 2.67 (t, 4H), | m/z 406.0 (M + H)⁺ (ES⁺) | B51 |
| | 2-((5-((1,2,3,5,6,7-hexahydro-s- | 2.04-1.96 (m, | | |
| | indacen-4-yl)amino)-4H-1,2,4-triazol- | 4H). One | | |
| | 3-yl)sulfonyl)benzonitrile | exchangeable | | |
| | | proton not | | |
| | | observed. | | |

### Example 186: 2-(4-((5-((1,2,3,5,6,7-hexahydro-s-indacen-4-yl)amino)-4H-1,2,4-triazol-3-yl) sulfonyl)phenyl) propan-2-01

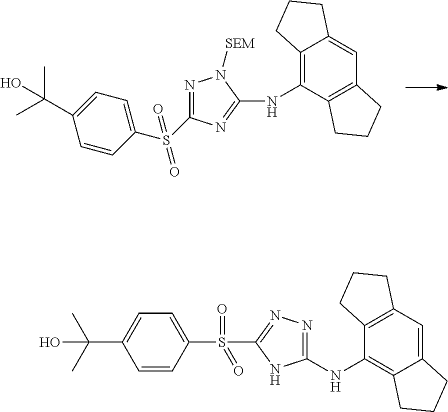 [SMILES: CC(C)(O)c1ccc(S(=O)(=O)c2nc(Nc3c4c(cc5c3CCC5)CCC4)n(COCC[Si](C)(C)C)n2)cc1 ; CC(C)(O)c1ccc(S(=O)(=O)c2nnc(Nc3c4c(cc5c3CCC5)CCC4)[nH]2)cc1]

To a solution of 2-(4-((5-((1,2,3,5,6,7-hexahydro-s-indacen-4-yl)amino)-1-((2-(trimethylsilyl)ethoxy)methyl)-1H-1,2,4-triazol-3-yl)sulfonyl)phenyl)propan-2-ol (Intermediate B39) (140 mg, 246.13 μmol, 1 eq) in DCM (4 mL) was added TFA (4 mL). The mixture was stirred at 25° C. for 4 hours. The reaction mixture was poured into ice water (20 mL) and extracted with DCM (3×20 mL). The organic layers were dried over anhydrous Na₂SO₄, filtered and concentrated in vacuum. The residue was purified by prep-HPLC (column: Waters Xbridge C18, 150 mm*25 mm*5 μm; mobile phase: [A: water (0.05% ammonia hydroxide v/v), B: MeCN]; B %: 10%-43%, 10 minutes) to give the title compound (49.16 mg, 45.54% yield, 100% purity on LCMS) as a white solid. ¹H NMR (400 MHz, CDCl₃): δ 8.04 (d, 2H), 7.69 (d, 2H), 7.01 (s, 1H), 2.88 (t, 4H), 2.68 (t, 4H), 2.05-1.97 (m, 4H), 1.60 (s, 6H). Three exchangeable protons not observed.

The following examples were synthesised following the general procedure for Example 186, from the intermediate compounds indicated in the ‘From’ column:

| | | ¹H NMR | | |
|---|---|---|---|---|
| Example | Structure | spectrum | LCMS | From |
| 187 | 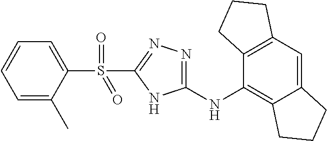 [SMILES: Cc1ccccc1S(=O)(=O)c1nnc(Nc2c3c(cc4c2CCC4)CCC3)[nH]1] | ¹H NMR (400 MHz, CDCl₃) δ 8.15 (d, 1H), 7.92 (s, 1H), 7.55 (t, 1H), 7.42-7.38 (m, 1H), 7.33 (d, 1H), 6.95 (s, 1H), 2.85-2.81 (m, 4H), 2.64-2.60 | m/z 395.0 (M + H)⁺ (ES⁺) | B38 |
| | N-(1,2,3,5,6,7-hexahydro-s- | (m, 7H), 2.00- | | |
| | indacen-4-yl)-5-(o-tolylsulfonyl)- | 1.92 (m, 4H). One | | |
| | 4H-1,2,4-triazol-3-amine | exchangeable | | |
| | | proton not | | |
| | | observed. | | |
| 188 | 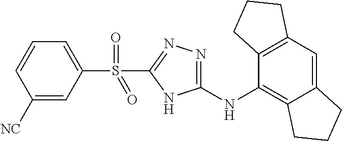 [SMILES: N#Cc1cccc(S(=O)(=O)c2nnc(Nc3c4c(cc5c3CCC5)CCC4)[nH]2)c1] | ¹H NMR (400 MHz, CDCl₃) δ 8.37 (s, 1H), 8.32 (d, 1H), 7.94 (d, 1H), 7.72 (t, 1H), 7.15 (br s, 1H), 7.04 (s, 1H), 2.90 (t, 4H), 2.67 (t, 4H), 2.08-2.00 | m/z 406.2 (M + H)⁺ (ES⁺); | B47 |
| | 3-((5-((1,2,3,5,6,7-hexahydro-s- | (m, 4H). One | | |
| | indacen-4-yl)amino)-4H-1,2,4- | exchangeable | | |
| | triazol-3-yl)sulfonyl)benzonitrile | proton not | | |
| | | observed. | | |

### Example 189: 5-(phenylsulfonyl)-N-(5-(pyridin-4-yl)-2,3-dihydro-1H-inden-4-yl)-4H-1,2,4-triazol-3-amine

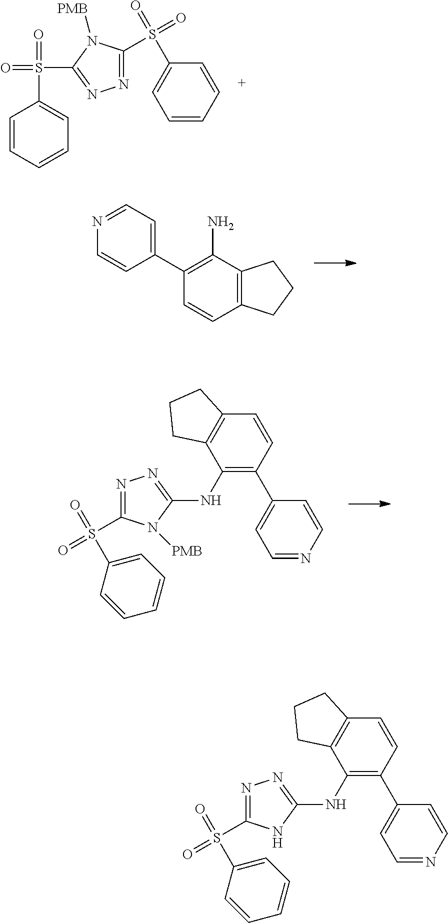

Step A: LiHMDS (1 M in THF, 0.426 mL, 0.426 mmol) was added dropwise to 44-methoxybenzyl)-3,5-bis(phenylsulfonyl)-4H-1,2,4-triazole (Intermediate A12) (100 mg, 0.213 mmol) and 5-(pyridin-4-yl)-2,3-dihydro-1H-inden-4-amine (Intermediate C4) (45 mg, 0.214 mmol) in THF (1 mL) at 0° C. The reaction was stirred at RT for 2 h and quenched with sat aq NH₄Cl (1 mL), extracted with EtOAc (30 mL), dried (phase separator) and concentrated in vacuo.

Step B: The residue was dissolved in TFA (4 mL), heated at 50° C. for 1 h and concentrated in vacuo. The crude was purified by acidic prep HPLC (20-50% MeOH in water) to afford the title compound (4 mg, 4%) as a white powder.

LCMS m/z 418.2 (M+H)⁺ (ES⁺); 416.4 (M−H)⁻ (ES⁻).

¹H NMR (DMSO-d₆) δ 13.15 (br s, 1H), 8.92 (s, 1H), 8.40 (dd, J=4.5, 1.7 Hz, 2H), 7.85-7.81 (m, 2H), 7.78-7.75 (m, 1H), 7.66 (t, J=7.9 Hz, 2H), 7.27-7.22 (m, 3H), 7.17 (d, J=7.0 Hz, 1H), 2.93 (t, J=7.5 Hz, 2H), 2.58 (t, J=7.4 Hz, 2H), 1.97 (p, J=7.5 Hz, 2H).

### Example 190: N-(5-(2-methoxypyridin-4-yl)-2,3-dihydrobenzofuran-4-yl)-5-(phenylsulfonyl)-4H-1,2,4-triazol-3-amine

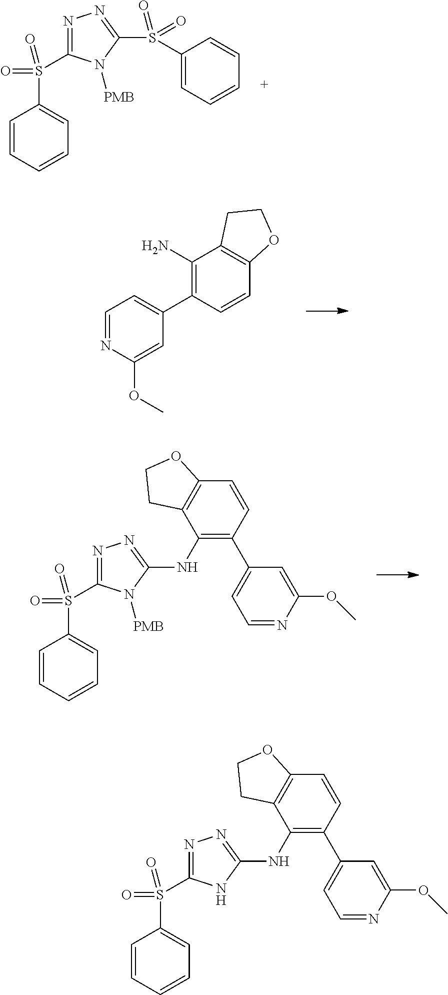

Step A: Prepared according to the general procedure of 5-(phenylsulfonyl)-N-(5-(pyridin-4-yl)-2,3-dihydro-1H-inden-4-yl)-4H-1,2,4-triazol-3-amine (Example 189, Step A) from 4-(4-methoxybenzyl)-3,5-bis(phenylsulfonyl)-4H-1,2,4-triazole (Intermediate A12) and 5-(2-methoxypyridin-4-yl)-2,3-dihydrobenzofuran-4-amine (Intermediate C5) to afford crude 4-(4-methoxybenzyl)-N-(5-(2-methoxypyridin-4-yl)-2,3-dihydrobenzofuran-4-yl)-5-(phenylsulfonyl)-4H-1,2,4-triazol-3-amine (208 mg, 91%) which was carried into the next step without further purification.

LCMS m/z 570.3 (M+H)+(ES+); 568.2 (M−H)− (ES−).

Step B: 4-(4-methoxybenzyl)-N-(5-(2-methoxypyridin-4-yl)-2,3-dihydrobenzofuran-4-yl)-5-(phenylsulfonyl)-4H-1,2,4-triazol-3-amine (110 mg, 0.193 mmol) was dissolved in a mixture of TFA (4 mL) and 1,3-dimethoxybenzene (0.13 mL, 0.993 mmol) and heated at 70° C. for 2 h, co-evaporated twice with toluene and purified by acidic prep HPLC (20-50% MeOH in water) to afford the title compound (12 mg, 13%) as a light tan solid.

LCMS m/z 450.3 (M+H)⁺ (ES⁺); 448.1 (M−H)⁻ (ES⁻).

¹H NMR (DMSO-d₆) δ 13.21 (br s, 1H), 9.12 (s, 1H), 7.98 (d, J=5.3 Hz, 1H), 7.88-7.85 (m, 2H), 7.76 (t, J=7.5 Hz, 1H), 7.66 (t, J=7.8 Hz, 2H), 7.15 (d, J=8.2 Hz, 1H), 6.81 (dd, J=5.3, 1.5 Hz, 1H), 6.77 (d, J=8.2 Hz, 1H), 6.63 (s, 1H), 4.52 (t, J=8.7 Hz, 2H), 3.78 (s, 3H), 2.86 (t, J=8.7 Hz, 2H).

### Example 191: N-(4-fluoro-2-isopropyl-6-(pyridin-4-yl)phenyl)-5-(phenylsulfonyl)-4H-1,2,4-triazol-3-amine

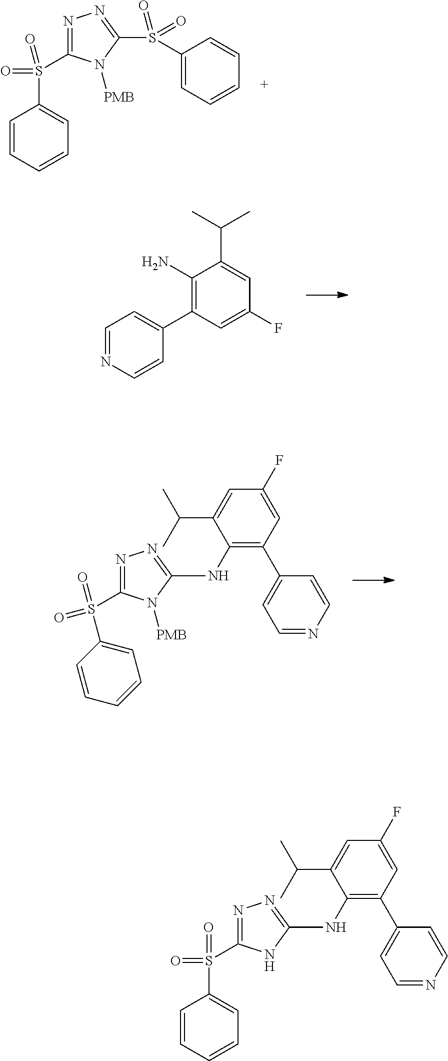

Prepared according to the general procedure of 5-(phenylsulfonyl)-N-(5-(pyridin-4-yl)-2,3-dihydro-1H-inden-4-yl)-4H-1,2,4-triazol-3-amine (Example 189) from 44-methoxybenzyl)-3,5-bis(phenylsulfonyl)-4H-1,2,4-triazole (Intermediate A12) and 4-fluoro-2-isopropyl-6-(pyridin-4-yl)aniline (Intermediate R11) to afford the title compound (35 mg, 19%) as a tan solid.

LCMS m/z 438.2 (M+H)⁺ (ES⁺); 436.1 (M−H)⁻(ES⁻).

¹H NMR (DMSO-d₆) δ 12.95 (br s, 1H), 8.92 (s, 1H), 8.39-8.35 (m, 2H), 7.80-7.72 (m, 3H), 7.64 (t, J=7.7 Hz, 2H), 7.31 (dd, J=9.9, 3.0 Hz, 1H), 7.24-7.20 (m, 2H), 7.11 (dd, J=8.7, 3.0 Hz, 1H), 3.08 (sept, J=6.6 Hz, 1H), 1.09 (d, J=6.9 Hz, 6H).

### Example 192: 6-(2-methoxypyridin-4-yl)-7-((5-(phenylsulfonyl)-4H-1,2,4-triazol-3-yl)amino)-2,3-dihydro-1H-indene-4-carbonitrile

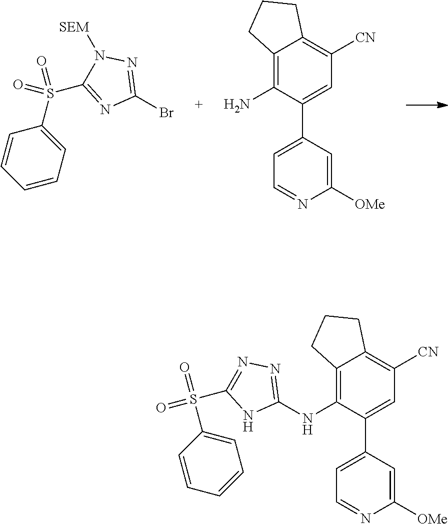 [SMILES: C[Si](C)(C)CCOCn1nc(Br)nc1S(=O)(=O)c1ccccc1 ; COc1cc(-c2cc(C#N)c3c(c2N)CCC3)ccn1 ; COc1cc(-c2cc(C#N)c3c(c2Nc2nnc(S(=O)(=O)c4ccccc4)[nH]2)CCC3)ccn1]

Prepared according to the general procedure of N-(1,2,3,5,6,7-hexahydro-s-indacen-4-yl)-5-(phenylsulfonyl)-1H-1,2,4-triazol-3-amine (Example 40) from 3-bromo-5-(phenylsulfonyl)-1-((2-(trimethylsilyl)ethoxy)methyl)-1H-1,2,4-triazole (Intermediate A1) and 7-amino-6-(2-methoxypyridin-4-yl)-2,3-dihydro-1H-indene-4-carbonitrile (Intermediate C6) to afford the title compound (13 mg, 9%) as a white solid.

LCMS m/z 473.3 (M+H)⁺ (ES⁺); 471.2 (M−H)⁻ (ES⁻).

¹H NMR (DMSO-d₆) δ 9.24 (s, 1H), 7.99 (d, J=5.3 Hz, 1H), 7.87-7.82 (m, 2H), 7.79-7.75 (m, 1H), 7.67 (t, J=7.8 Hz, 2H), 7.61 (s, 1H), 6.82 (dd, J=5.3, 1.5 Hz, 1H), 6.69 (d, J=1.4 Hz, 1H), 3.80 (s, 3H), 3.06 (t, J=7.5 Hz, 2H), 2.58 (t, J=7.4 Hz, 2H), 2.02 (p, J=7.6 Hz, 2H). One exchangeable proton not observed.

### Example 13: 3-((1-cyclopropyl-H-pyrazol-4-yl)sulfonyl)-N-(1,2,3,5,6,7-hexahydro-s-indacen-4-yl)-1H-1,2,4-triazol-5-amine

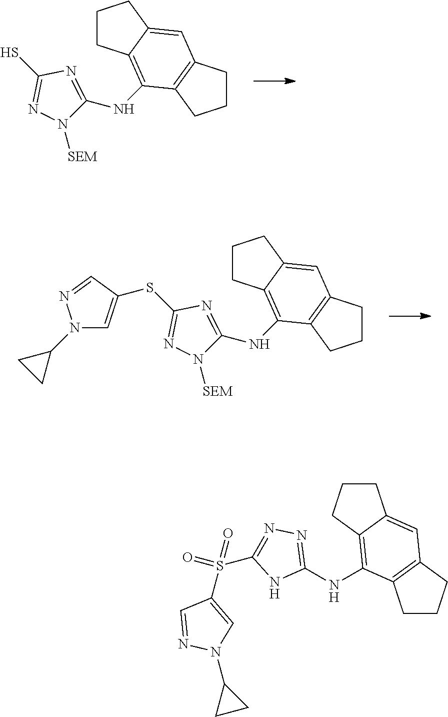 [SMILES: C[Si](C)(C)CCOCn1nc(S)nc1Nc1c2c(cc3c1CCC3)CCC2 ; C[Si](C)(C)CCOCn1nc(Sc2cnn(C3CC3)c2)nc1Nc1c2c(cc3c1CCC3)CCC2 ; O=S(=O)(c1cnn(C2CC2)c1)c1nnc(Nc2c3c(cc4c2CCC4)CCC3)[nH]1]

Step A: To 5-((1,2,3,5,6,7-hexahydro-s-indacen-4-yl)amino)-1-((2-(trimethylsilyl)-ethoxy)methyl)-1H-1,2,4-triazole-3-thiol (Intermediate B9) (0.3 g, 0.484 mmol) and CuI (10.0 mg, 0.052 mmol) under N₂ was added N¹,N²-dimethylethane-1,2-diamine (11.00 μL, 0.102 mmol) and 1-cyclopropyl-4-iodo-1H-pyrazole (0.113 g, 0.484 mmol) in dioxane (1 mL. The reaction was degassed, heated at reflux for 20 h, diluted with EtOAc (10 mL) and washed with water (10 mL). The aqueous layer was then re-extracted with EtOAc (2×10 mL) and the combined organics were dried (MgSO₄) and then concentrated in vacuo. The crude product was purified by chromatography on silica gel (4 g column, 0-75% EtOAc/isohexane) to afford 3-((1-cyclopropyl-H-pyrazol-4-yl)thio)-N-(1,2,3,5,6,7-hexahydro-s-indacen-4-yl)-1-((2-(trimethylsilyl)ethoxy)-methyl)-1H-1,2,4-triazol-5-amine (94 mg 38%) as a pale orange solid.

¹H NMR (methanol-d₄) δ 8.00-7.71 (m, 1H), 7.65-7.41 (m, 1H), 6.98-6.92 (m, 1H), 5.38-5.13 (m, 2H), 3.74-3.52 (m, 3H), 2.97-2.76 (m, 4H), 2.73-2.56 (m, 4H), 2.32-1.85 (m, 4H), 1.22-0.98 (m, 4H), 0.96-0.81 (m, 2H), 0.16-−0.22 (m, 9H). One exchangeable proton not observed.

Step B: Prepared according to the general procedure of N-(4-fluoro-2,6-diisopropylphenyl)-3-(phenylsulfonyl)-1H-1,2,4-triazol-5-amine (Example 42, Steps B and C) from 3-((1-cyclopropyl-H-pyrazol-4-yl)thio)-N-(1,2,3,5,6,7-hexahydro-s-indacen-4-yl)-1-((2-(trimethylsilyl)ethoxy)methyl)-1H-1,2,4-triazol-5-amine to afford the title compound (7 mg, 9%) as a pale yellow solid.

LCMS m/z 411.2 (M+H)⁺ (ES⁺).

¹H NMR (methanol-d₄) δ 8.35 (s, 1H), 7.86 (s, 1H), 6.99 (s, 1H), 3.80-3.74 (m, 1H), 2.87 (t, J=7.4 Hz, 4H), 2.67 (t, J=7.3 Hz, 4H), 2.03 (p, J=7.4 Hz, 4H), 1.19-1.14 (m, 2H), 1.10-1.05 (m, 2H). Two exchangeable proton not observed.

### Example 14: N-(1,2,3,5,6,7-hexahydro-s-indacen-4-yl)-3-((3-(pyrrolidin-1-yl)cyclohexyl)sulfonyl)-1H-1,2,4-triazol-5-amine

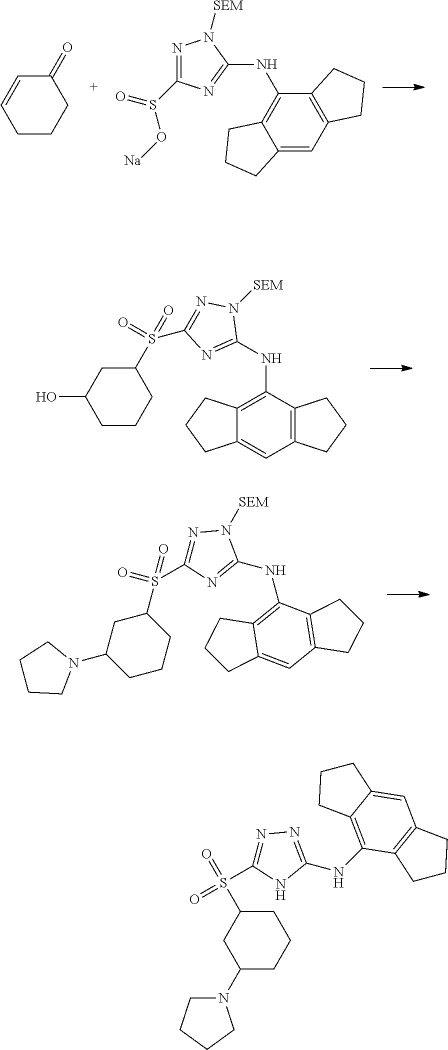

Step A: Sodium 5-((1,2,3,5,6,7-hexahydro-s-indacen-4-yl)amino)-1-((2-(trimethylsilyl)-ethoxy)methyl)-1H-1,2,4-triazole-3-sulfinate (Intermediate B2) (750 mg, 1.64 mmol) was suspended in cyclohex-2-enone (3.190 mL, 32.9 mmol) and cooled to 0° C. TMS-C1 (252 μL, 1.97 mmol) was added and the reaction stirred at ° C. for 1 h. NaBH₄ (81 mg, 2.14 mmol) was added, the reaction was warmed to RT and quenched by slow addition of sat aq NH₄Cl (100 mL). The product was extracted with EtOAc (2×75 mL) and the organics combined, dried (phase separator) and concentrated in vacuo. The product was purified by chromatography on silica gel (24 g column, 0-75% EtOAc/isohexane) to afford 3-((5-((1,2,3,5,6,7-hexahydro-s-indacen-4-yl)amino)-1-((2-(trimethylsilyl)-ethoxy)methyl)-1H-1,2,4-triazol-3-yl)sulfonyl)cyclohexanol (622 mg, 70%) as a light yellow crystalline solid.

LCMS m/z 533.4 (M+H)⁺ (ES⁺); 531.3 (M−H)⁻ (ES⁻).

¹H NMR (DMSO-d₆) δ 8.89 (s, 1H), 6.98 (s, 1H). 5.54 (s, 1H), 4.84 (d, J=4.6 Hz, 1H), 3.61 (t, J=8.1 Hz, 2H), 3.45-3.37 (m, 1H), 3.30-3.23 (m, 1H), 2.83 (t, J=7.4 Hz, 4H), 2.64 (t, J=7.4 Hz, 4H), 2.11-2.05 (m, 1H), 1.97 (p, J=7.4 Hz, 4H), 1.82-1.70 (m, 3H), 1.29-1.14 (m, 3H), 1.03-095 (m, 1H), 0.88 (t, J=8.1 Hz, 2H), −0.03 (s, 9H). One exchangeable proton not observed.

Step B: 3-((5-((1,2,3,5,6,7-Hexahydro-s-indacen-4-yl)amino)-1-((2-(trimethylsilyl)-ethoxy)methyl)-1H-1,2,4-triazol-3-yl)sulfonyl)cyclohexanol (100 mg, 0.188 mmol) and DIPEA (52 μL, 0.298 mmol) were dissolved in THF (2 mL) and cooled to 0° C. MsCl (19 μL, 0.244 mmol) was added and the reaction was warm to RT, then stirred for 2 h. KI (2 mg, 0.012 mmol) and pyrrolidine (267 mg, 3.75 mmol) were added, the reaction vessel was sealed and the reaction heated to 6° C., then stirred for 48 h, diluted with water (30 mL) and extracted with EtOAc (2×30 mL). The organics were dried (phase separator) and concentrated in vacuo.

Step C: The residue obtained in Step B was dissolved in HCl (4 M in dioxane, 2 mL, 8.00 mmol) and stirred at RT for 16 h. The reaction was concentrated in vacuo and purified by acidic prep HPLC (35-65% MeOH in water) to afford the title compound (3 mg, 3%) as a white solid.

LCMS m/z 456.4 (M+H)⁺ (ES⁺); 454.3 (M−H)⁻ (ES⁻).

¹H NMR (DMSO-d₆) δ. 9.03 (s, 1H), 6.94 (s, 1H), 2.95-2.87 (m, 1H), 2.82 (t, J=7.4 Hz, 4H), 2.65 (t, J=7.3 Hz, 4H), 2.37 (t, J=6.2 Hz, 4H), 2.12-2.03 (m, 1H), 1.96 (p, 4H), 1.82-1.73 (m, 2H), 1.72-1.58 (m, 2H), 1.52-1.38 (m, 3H), 1.38-1.14 (m, 5H). One exchangeable proton not observed.

### Example 195: N-(5-(2-(methoxy-d)pyridin-4-yl)-2,3-dihydrobenzofuran-4-yl)-5-((6-methyl-2,6-diazaspiro[3-4]octan-2-yl)sulfonyl)-4H-1,2,4-triazol-3-amine

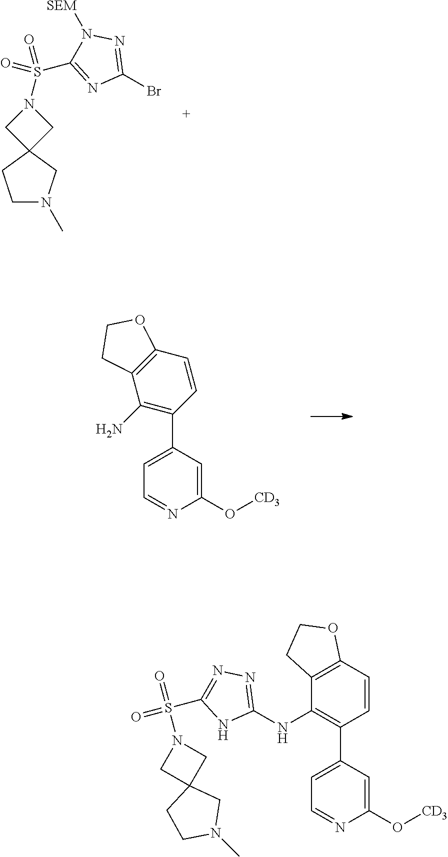 [SMILES: CN1CCC2(C1)CN(S(=O)(=O)c1nc(Br)nn1COCC[Si](C)(C)C)C2 ; [2H]C([2H])([2H])Oc1cc(-c2ccc3c(c2N)CCO3)ccn1 ; [2H]C([2H])([2H])Oc1cc(-c2ccc3c(c2Nc2nnc(S(=O)(=O)N4CC5(CCN(C)C5)C4)[nH]2)CCO3)ccn1]

A solution of 2-((3-bromo-1-((2-(trimethylsilyl)ethoxy)methyl)-1H-1,2,4-triazol-5-yl)-sulfonyl)-6-methyl-2,6-diazaspiro[3.4]octane (Intermediate A13) (115 mg, 0.247 mmol) in 1,4-dioxane (4 mL) was added to a mixture of 5-(2-(methoxy-d₃)pyridin-4-yl)-2,3-dihydrobenzofuran-4-amine (66.5 mg, 0.271 mmol) (Intermediate C22), Pd-175 (38.5 mg, 0.049 mmol) and K₂CO₃ (102 mg, 0.740 mmol) under N₂, the solution was evacuated and backfilled with N₂ (3 times) and stirred at 87° C. for 16 h. The solution was filtered through a plug of Celite, rinsing with EtOAc (40 mL) and the crude product was directly loaded onto silica. The product was purified by chromatography on silica gel (12 g column, 0-10% (0.7 M Ammonia/MeOH)/DCM) and the product was taken up in TFA (2 mL) and stirred at RT for 30 min. The volatiles were removed in vacuo and the crude product was purified by acidic prep HPLC (10-40% MeOH in water) to afford the title compound (32 mg, 26%) as a white solid.

LCMS m/z 501.3 (M+H)⁺ (ES⁺).

¹H NMR (DMSO-d₆) δ 13.38 (s, 1H), 9.22 (s, 1H), 8.11 (d, J=5.3 Hz, 1H), 7.22 (d, J=8.2 Hz, 1H), 6.93 (dd, J=5.3, 1-3 Hz, 1H), 6.84 (d, J=8.2 Hz, 1H), 6.75-6.71 (m, 1H), 4.62 (t, J=8.7 Hz, 2H), 4.02-3.92 (m, 3H), 3.83 (d, J=8.7 Hz, 1H), 3.70-3.60 (m, 1H), 3.56-3.45 (m, 1H), 3.15-3.09 (m, 1H), 3.08-2.96 (m, 3H), 2.77 (app. d, J=4.1 Hz, 3H), 2.18-2.09 (m, 1H), 2.00-1.87 (m, 1H).

### Example 196:N-(5-(2-(methoxy-d)pyridin-4-yl)-2,3-dihydro-1H-inden-4-yl)-5-((6-methyl-2,6-diazaspiro[3.4]octan-2-yl)sulfonyl)-4H-1,2,4-triazol-3-amine

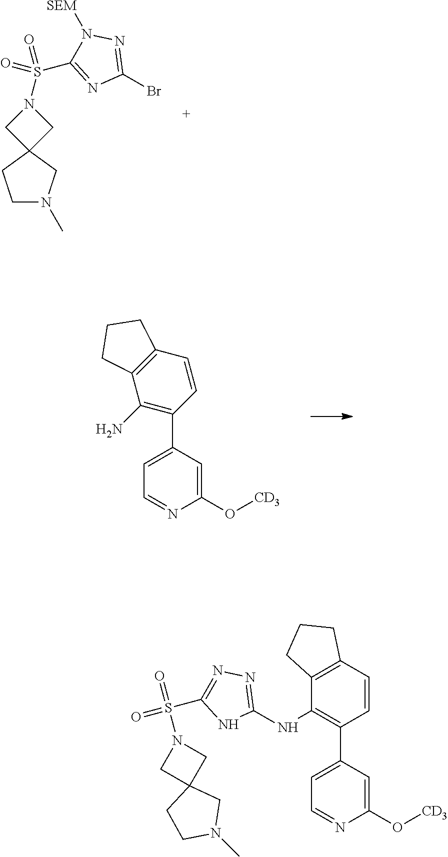 [SMILES: CN1CCC2(C1)CN(S(=O)(=O)c1nc(Br)nn1COCC[Si](C)(C)C)C2 ; [2H]C([2H])([2H])Oc1cc(-c2ccc3c(c2N)CCC3)ccn1 ; [2H]C([2H])([2H])Oc1cc(-c2ccc3c(c2Nc2nnc(S(=O)(=O)N4CC5(CCN(C)C5)C4)[nH]2)CCC3)ccn1]

Prepared according to the general procedure of N-(5-(2-(methoxy-d)pyridin-4-yl)-2,3-dihydrobenzofuran-4-yl)-5-((6-methyl-2,6-diazaspiro[3-4]octan-2-yl)sulfonyl)-4H-1,2,4-triazol-3-amine (Example 195) from 2-((3-bromo-1-((2-(trimethylsilyl)ethoxy)-methyl)-1H-1,2,4-triazol-5-yl)sulfonyl)-6-methyl-2,6-diazaspiro[3-4]octane (Intermediate A13) and 5-(2-(methoxy-d₃)pyridin-4-yl)-2,3-dihydro-1H-inden-4-amine (Intermediate C21) to afford the title compound (8 mg, 6%) as a white solid.

LCMS m/z 499.3 (M+H)⁺ (ES⁺).

¹H NMR (DMSO-d₆) δ 9.05 (s, 1H), 8.12 (d, J=5.3 Hz, 1H), 7.29 (d, J=7.7 Hz, 1H), 7.22 (d, J=7.7 Hz, 1H), 6.94 (d, J=5.3 Hz, 1H), 6.75 (s, 1H), 3.91 (d, J=8.6 Hz, 2H), 3.83 (d, J=8.6 Hz, 2H), 3.07 (brs, 2H), 2.97 (t, J=7.4 Hz, 2H), 2.71 (t, J=7.4 Hz, 2H), 2.59 (br s, 3H), 2.04 (p, J=7.4 Hz, 2H), 1.94 (t, J=6.3 Hz, 2H). One exchangeable proton not observed. Two protons obscured by solvent.

### Example 197:1-((5-((4-isopropyl-1-(pyridin-4-yl)-1H-pyrazol-5-yl)amino)-4H-1,2,4-triazol-3-yl)sulfonyl)-N,N-dimethylpiperidin-4-amine

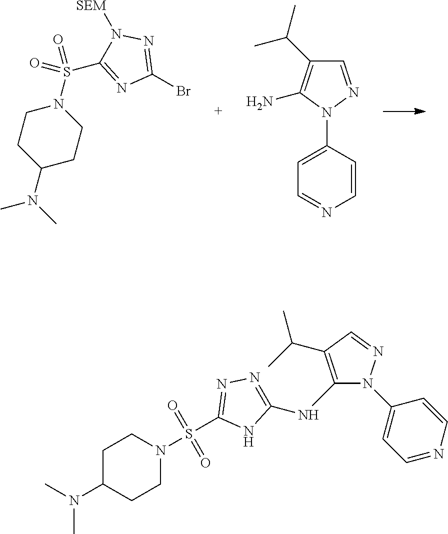 [SMILES: CN(C)C1CCN(S(=O)(=O)c2nc(Br)nn2COCC[Si](C)(C)C)CC1 ; CC(C)c1cnn(-c2ccncc2)c1N ; CC(C)c1cnn(-c2ccncc2)c1Nc1nnc(S(=O)(=O)N2CCC(N(C)C)CC2)[nH]1]

Prepared according to the general procedure of N-(5-(2-(methoxy-d)pyridin-4-yl)-2,3-dihydrobenzofuran-4-yl)-5-((6-methyl-2,6-diazaspiro[3.4]octan-2-yl)sulfonyl)-4H-1,2,4-triazol-3-amine (Example 195) from 1-((3-bromo-1-((2-(trimethylsilyl)ethoxy)-methyl)-1H-1,2,4-triazol-5-yl)sulfonyl)-N,N-dimethylpiperidin-4-amine (Intermediate A11) and 4-isopropyl-1-(pyridin-4-yl)-H-pyrazol-5-amine (Intermediate C27) to afford the title compound (5 mg, 5%) as a white solid.

LCMS m/z 460.3 (M+H)⁺ (ES⁺).

¹H NMR (DMSO-d₆) δ 13.72 (s, 1H), 9.70 (s, 1H), 8.67-8.59 (m, 2H), 7.87 (s, 1H), 7.72-7.63 (m, 2H), 3.69 (br d, J=12.2 Hz, 2H), 3.17-3.08 (m, 1H), 2.78-2.67 (m, 7H), 2.38-2.31 (m, 2H), 2.01 (brd, J=12.0 Hz, 2H), 1.69-1.58 (m, 2H), 1.16 (d, J=6.9 Hz, 6H).

### Example 18: 1-((5-((1-isopropyl-4-(pyridin-4-yl)-1H-pyrazol-5-yl)amino)-4H-1,2,4-triazol-3-yl)sulfonyl)-N,N-dimethylpiperidin-4-amine

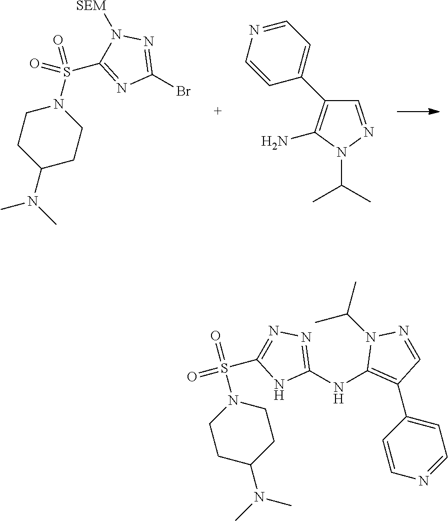 [SMILES: CN(C)C1CCN(S(=O)(=O)c2nc(Br)nn2COCC[Si](C)(C)C)CC1 ; CC(C)n1ncc(-c2ccncc2)c1N ; CC(C)n1ncc(-c2ccncc2)c1Nc1nnc(S(=O)(=O)N2CCC(N(C)C)CC2)[nH]1]

Prepared according to the general procedure of N-(5-(2-(methoxy-d)pyridin-4-yl)-2,3-dihydrobenzofuran-4-yl)-5-((6-methyl-2,6-diazaspiro[3-4]octan-2-yl)sulfonyl)-4H-1,2,4-triazol-3-amine (Example 195) from 1-((3-bromo-1-((2-(trimethylsilyl)ethoxy)-methyl)-1H-1,2,4-triazol-5-yl)sulfonyl)-N,N-dimethylpiperidin-4-amine (Intermediate A11) and 1-isopropyl-4-(pyridin-4-yl)-1H-pyrazol-5-amine (Intermediate C26) to afford the title compound (6 mg, 6%) as a white solid.

LCMS m/z 460.3 (M+H)⁺ (ES⁺).

¹H NMR (DMSO-d₆) δ 9.70 (s, 1H), 8.51-8.42 (m, 2H), 8.15 (s, 1H), 8.10 (s, 1H), 7.48-7.41 (m, 2H), 4.54 (p, J=6.6 Hz, 1H), 3.62 (br d, J=12.4 Hz, 2H), 2.47-2.38 (m, 8H), 1.85 (br d, J=12.5 Hz, 2H), 1.54-1.44 (m, 2H), 1.38 (d, J=6.6 Hz, 6H). One proton obscured by solvent.

### Example 19: 1-((5-((5-(2-cyclopropylpyridin-4-yl)-2,3-dihydro-1H-inden-4-yl)amino)-4H-1,2,4-triazol-3-yl)sulfonyl)-N,N-dimethylpiperidin-4-amine

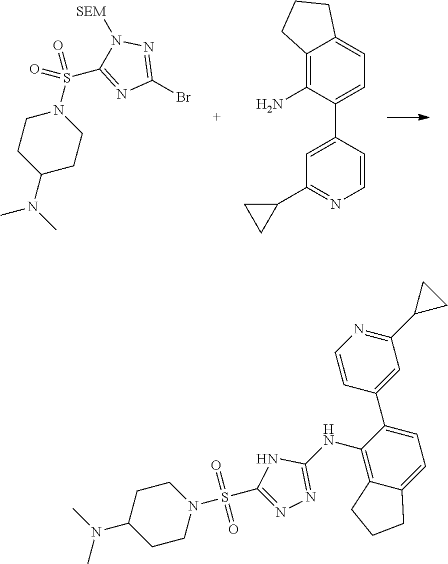 [SMILES: CN(C)C1CCN(S(=O)(=O)c2nc(Br)nn2COCC[Si](C)(C)C)CC1 ; Nc1c(-c2ccnc(C3CC3)c2)ccc2c1CCC2 ; CN(C)C1CCN(S(=O)(=O)c2nnc(Nc3c(-c4ccnc(C5CC5)c4)ccc4c3CCC4)[nH]2)CC1]

Prepared according to the general procedure of N-(5-(2-(methoxy-d)pyridin-4-yl)-2,3-dihydrobenzofuran-4-yl)-5-((6-methyl-2,6-diazaspiro[3.4]octan-2-yl)sulfonyl)-4H-1,2,4-triazol-3-amine (Example 195) from 1-((3-bromo-1-((2-(trimethylsilyl)ethoxy)-methyl)-1H-1,2,4-triazol-5-yl)sulfonyl)-N,N-dimethylpiperidin-4-amine (Intermediate A11) and 5-(2-cyclopropylpyridin-4-yl)-2,3-dihydro-1H-inden-4-amine (Intermediate C15) to afford the title compound (23 mg, 21%) as a white solid.

LCMS m/z 508.3 (M+H)⁺ (ES⁺).

¹H NMR (DMSO-d₆) δ 13.15 (s, 1H), 9.73 (s, 1H), 9.06 (s, 1H), 8.41 (d, J=5.4 Hz, 1H), 7.30 (d, J=7.7 Hz, 1H), 7.26 (d, J=7.7 Hz, 1H), 7.23 (br s, 1H), 3.74 (br d, J=12.4 Hz, 2H), 3.25 (t, J=12.3 Hz, 1H), 2.98 (t, J=7.4 Hz, 2H), 2.76-2.73 (m, 8H), 2.13-2.02 (m, 5H), 1.67 (qd, J=12.1, 4.0 Hz, 2H), 1.03 (d, J=7.9 Hz, 2H), 0.94-0.88 (m, 2H). Two protons obscured by solvent.

### Example 200: N-(5-(2-(difluoromethoxy)pyridin-4-yl)-2,3-dihydro-1H-inden-4-yl)-5-((1-methyl-1,7-diazaspiro[3.5]nonan-7-yl)sulfonyl)-4H-1,2,4-triazol-3-amine

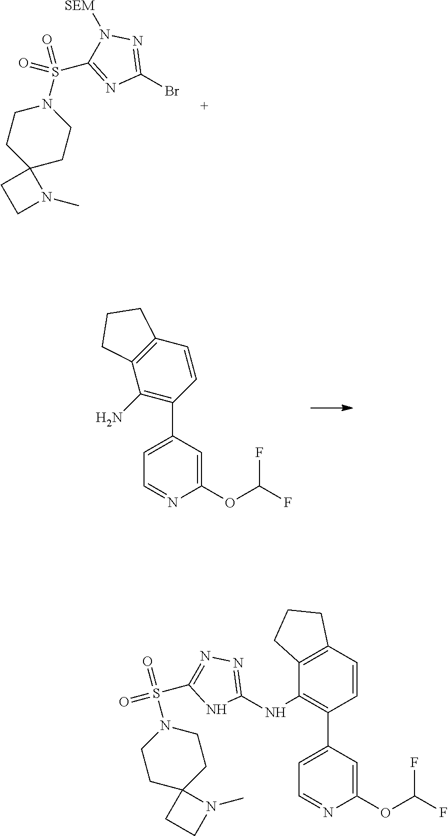 [SMILES: CN1CCC12CCN(S(=O)(=O)c1nc(Br)nn1COCC[Si](C)(C)C)CC2 ; Nc1c(-c2ccnc(OC(F)F)c2)ccc2c1CCC2 ; CN1CCC12CCN(S(=O)(=O)c1nnc(Nc3c(-c4ccnc(OC(F)F)c4)ccc4c3CCC4)[nH]1)CC2]

Prepared according to the general procedure of N-(5-(2-(methoxy-d)pyridin-4-yl)-2,3-dihydrobenzofuran-4-yl)-5-((6-methyl-2,6-diazaspiro[3.4]octan-2-yl)sulfonyl)-4H-1,2,4-triazol-3-amine (Example 195) from 7-((3-bromo-1-((2-(trimethylsilyl)ethoxy)-methyl)-1H-1,2,4-triazol-5-yl)sulfonyl)-1-methyl-1,7-diazaspiro[3.5]nonane (Intermediate A14) and 5-(2-(difluoromethoxy)pyridin-4-yl)-2,3-dihydro-1H-inden-4-amine (Intermediate C8) to afford the title compound (5 mg, 6%) as a white solid.

LCMS m/z 546.3 (M+H)⁺ (ES⁺).

¹H NMR (DMSO-d₆) δ 9.12 (s, 1H), 8.20 (d, J=5.3 Hz, 1H), 7.70 (t, J=72.9 Hz, 1H), 7.29 (d, J=7.7 Hz, 1H), 7.25 (d, J=7.7 Hz, 1H), 7.20 (dd, J=5.3, 1.4 Hz, 1H), 6.99 (d, J=1.3 Hz, 1H), 3.57 (br d, J=12.2 Hz, 2H), 3.28-3.21 (m, 2H), 2.96 (t, J=7.4 Hz, 2H), 2.70 (t, J=7.4 Hz, 2H), 2.62-2.53 (m, 2H), 2.18 (s, 3H), 2.03 (p, J=7.5 Hz, 2H), 1.92-1.79 (m, 4H), 1.65 (br t, J=13.9 Hz, 2H). One exchangeable proton not observed.

### Example 201: N-(5-(2-(difluoromethoxy)pyridin-4-yl)-2,3-dihydro-1H-inden-4-yl)-5-((1-methyl-1,8-diazaspiro[4.5]decan-8-yl)sulfonyl)-4H-1,2,4-triazol-3-amine

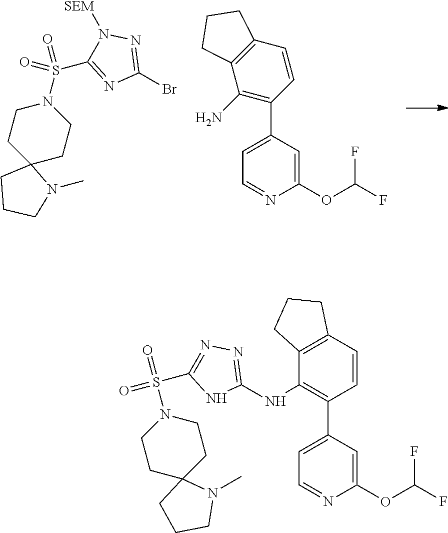 [SMILES: CN1CCCC12CCN(S(=O)(=O)c1nc(Br)nn1COCC[Si](C)(C)C)CC2 ; Nc1c(-c2ccnc(OC(F)F)c2)ccc2c1CCC2 ; CN1CCCC12CCN(S(=O)(=O)c1nnc(Nc3c(-c4ccnc(OC(F)F)c4)ccc4c3CCC4)[nH]1)CC2]

Prepared according to the general procedure of N-(5-(2-(methoxy-d₃)pyridin-4-yl)-2,3-dihydrobenzofuran-4-yl)-5-((6-methyl-2,6-diazaspiro[3.4]octan-2-yl)sulfonyl)-4H-1,2,4-triazol-3-amine (Example 195) from 8-((3-bromo-1-((2-(trimethylsilyl)ethoxy)-methyl)-1H-1,2,4-triazol-5-yl)sulfonyl)-1-methyl-1,8-diazaspiro[4.5]decane (Intermediate A15) and 5-(2-(difluoromethoxy)pyridin-4-yl)-2,3-dihydro-1H-inden-4-amine (Intermediate C8) to afford the title compound (40 mg, 22%) as a white solid.

LCMS m/z 560.3 (M+H)⁺ (ES⁺).

¹H NMR (DMSO-d₆) δ 13-19 (s, 1H), 9.08 (s, 1H), 8.21 (d, J=5.3 Hz, 1H), 7.69 (t, J=72.9 Hz, 1H), 7.29 (d, J=7.7 Hz, 1H), 7.25 (d, J=7.7 Hz, 1H), 7.20 (dd, J=5.3, 1.4 Hz, 1H), 6.99 (d, J=1.3 Hz, 1H), 3.73-3.67 (m, 2H), 3.24 (br s, 2H), 2.97 (t, J=7.4 Hz, 2H), 2.75-2.64 (m, 4H), 2.58 (s, 3H), 2.04 (p, J=7.5 Hz, 2H), 1.95-1.80 (m, 6H), 1.64 (br d, J=12.4 Hz, 2H).

### Example 202: N-(5-(2-(difluoromethoxy)pyridin-4-yl)-2,3-dihydrobenzofuran-4-yl)-5-((1-methyl-1,7-diazaspiro[3.5]nonan-7-yl)sulfonyl)-4H-1,2,4-triazol-3-amine

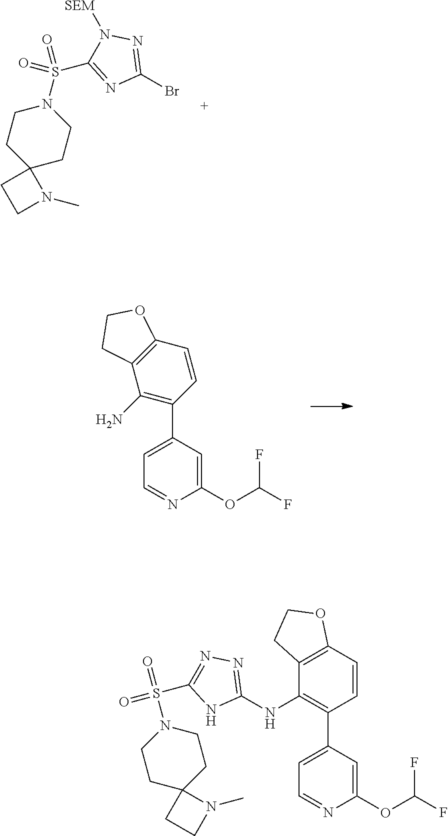 [SMILES: CN1CCC12CCN(S(=O)(=O)c1nc(Br)nn1COCC[Si](C)(C)C)CC2 ; Nc1c(-c2ccnc(OC(F)F)c2)ccc2c1CCO2 ; CN1CCC12CCN(S(=O)(=O)c1nnc(Nc3c(-c4ccnc(OC(F)F)c4)ccc4c3CCO4)[nH]1)CC2]

Prepared according to the general procedure of N-(5-(2-(methoxy-d)pyridin-4-yl)-2,3-dihydrobenzofuran-4-yl)-5-((6-methyl-2,6-diazaspiro[3.4]octan-2-yl)sulfonyl)-4H-1,2,4-triazol-3-amine (Example 195) from 7-((3-bromo-1-((2-(trimethylsilyl)ethoxy)-methyl)-1H-1,2,4-triazol-5-yl)sulfonyl)-1-methyl-1,7-diazaspiro[3.5]nonane (Intermediate A14) and 5-(2-(difluoromethoxy)pyridin-4-yl)-2,3-dihydrobenzofuran-4-amine (Intermediate C24) to afford the title compound (48 mg, 45%) as a white solid.

LCMS m/z 548.3 (M+H)⁺ (ES⁺); 546.2 (M−H)⁻ (ES⁻).

¹H NMR (DMSO-d₆) δ 8.10 (d, J=5.3 Hz, 1H), 7.53 (t, J=72.9 Hz, 1H), 7.22 (d, J=8.3 Hz, 1H), 7.17 (dd, J=5.3, 1.5 Hz, 1H), 6.90 (br s, 1H), 6.81 (d, J=8.3 Hz, 1H), 4.58 (t, J=8.7 Hz, 2H), 4.07-3.99 (m, 1H), 3.83-3.70 (m, 1H), 3.65 (br d, J=12.4 Hz, 1H), 3.56 (br d, J=12.8 Hz, 1H), 3.04 (t, J=8.7 Hz, 2H), 2.64-2.54 (m, 4H), 2.33-2.16 (m, 3H), 2.13-2.05 (m, 1H), 1.98-1.86 (m, 2H). One aliphatic proton obscured by solvent. Two exchangeable protons not observed.

### Example 203: N-(5-(2-methoxypyridin-4-yl)-2,3-dihydrobenzofuran-4-yl)-5-((1-methyl-1,7-diazaspiro[3.5]nonan-7-yl)sulfonyl)-4H-1,2,4-triazol-3-amine

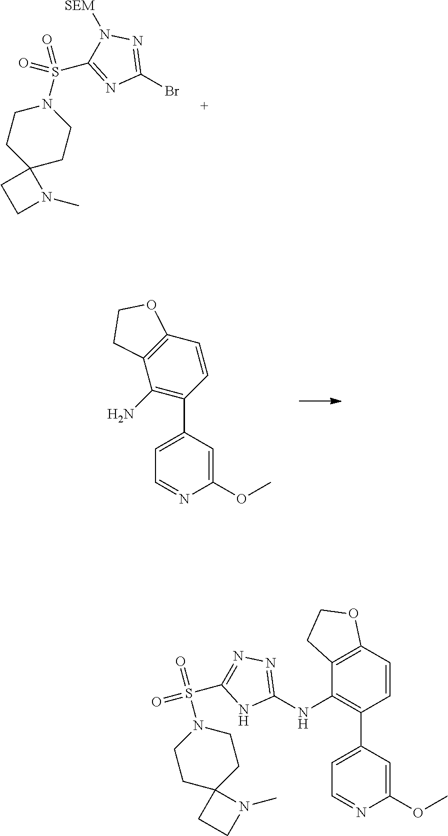 [SMILES: CN1CCC12CCN(S(=O)(=O)c1nc(Br)nn1COCC[Si](C)(C)C)CC2 ; COc1cc(-c2ccc3c(c2N)CCO3)ccn1 ; COc1cc(-c2ccc3c(c2Nc2nnc(S(=O)(=O)N4CCC5(CCN5C)CC4)[nH]2)CCO3)ccn1]

Prepared according to the general procedure of N-(5-(2-(methoxy-d)pyridin-4-yl)-2,3-dihydrobenzofuran-4-yl)-5-((6-methyl-2,6-diazaspiro[3-4]octan-2-yl)sulfonyl)-4H-1,2,4-triazol-3-amine (Example 195) from 7-((3-bromo-1-((2-(trimethylsilyl)ethoxy)-methyl)-1H-1,2,4-triazol-5-yl)sulfonyl)-1-methyl-1,7-diazaspiro[3.5]nonane (Intermediate A14) and 5-(2-methoxypyridin-4-yl)-2,3-dihydrobenzofuran-4-amine (Intermediate C5) to afford the title compound (40 mg, 40%) as a white solid.

LCMS m/z 512.3 (M+H)⁺ (ES⁺); 510.2 (M−H)⁻ (ES⁻).

¹H NMR (DMSO-d₆) δ 8.03 (d, J=5.4 Hz, 1H), 7.17 (d, J=8.2 Hz, 1H), 6.89 (dd, J=5-4, 1.5 Hz, 1H), 6.79 (d, J=8.3 Hz, 1H), 6.68 (br s, 1H), 4.57 (t, J=8.7 Hz, 2H), 4.08-4.00 (m, 1H), 3.79 (s, 3H), 3.66 (br d, J=12.4 Hz, 1H), 3.59 (br d, J=12.7 Hz, 1H), 3.01 (t, J=8.7 Hz, 2H), 2.66-2.51 (m, 5H), 2.33-2.21 (m, 3H), 2.17-2.07 (m, 1H), 1.98-1.88 (m, 2H). One aliphatic proton obscured by solvent. Two exchangeable protons not observed.

### Example 204:N-(5-(2-methoxypyridin-4-yl)-2,3-dihydro-1H-inden-4-yl)-5-((1-methyl-1,7-diazaspiro[3.5]nonan-7-yl)sulfonyl)-4H-1,2,4-triazol-3-amine

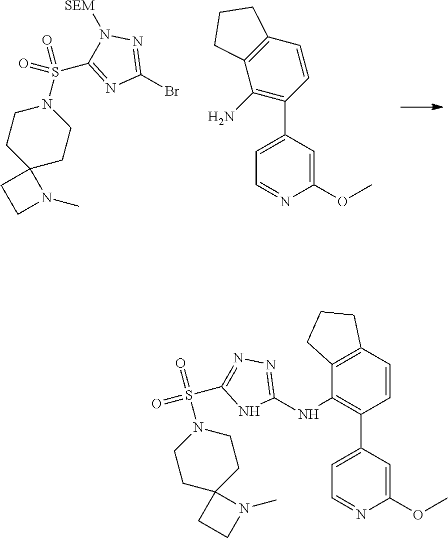 [SMILES: CN1CCC12CCN(S(=O)(=O)c1nc(Br)nn1COCC[Si](C)(C)C)CC2 ; COc1cc(-c2ccc3c(c2N)CCC3)ccn1 ; COc1cc(-c2ccc3c(c2Nc2nnc(S(=O)(=O)N4CCC5(CCN5C)CC4)[nH]2)CCC3)ccn1]

Prepared according to the general procedure of N-(5-(2-(methoxy-d₃)pyridin-4-yl)-2,3-dihydrobenzofuran-4-yl)-5-((6-methyl-2,6-diazaspiro[3.4]octan-2-yl)sulfonyl)-4H-1,2,4-triazol-3-amine (Example 195) from 7-((3-bromo-1-((2-(trimethylsilyl)ethoxy)-methyl)-1H-1,2,4-triazol-5-yl)sulfonyl)-1-methyl-1,7-diazaspiro[3.5]nonane (Intermediate A14) and 5-(2-methoxypyridin-4-yl)-2,3-dihydro-1H-inden-4-amine (Intermediate R4) to afford the title compound (29 mg, 29%) as a white solid. LCMS m/z 510.4 (M+H)⁺ (ES⁺); 508.2 (M−H)⁻ (ES⁻).

¹H NMR (DMSO-d₆) δ 8.98 (s, 1H), 8.10 (d, J=5.3 Hz, 1H), 7.27 (d, J=7.7 Hz, 1H), 7.20 (d, J=7.7 Hz, 1H), 6.91 (dd, J=5.3, 1.4 Hz, 1H), 6.75-6.69 (m, 1H), 3.85-3.76 (m, 5H), 3.64 (br d, J=12.8 Hz, 2H), 2.95 (t, J=7.4 Hz, 2H), 2.69 (t, J=7.4 Hz, 2H), 2.64-2.55 (m, 5H), 2.29-2.11 (m, 4H), 2.02 (p, J=7.5 Hz, 2H), 1.92 (td, J=12.3, 6.2 Hz, 2H). One exchangeable proton not observed.

### Example 205: N,N-dimethyl-1-((5-((5-(1-methyl-1H-pyrazol-5-yl)-2,3-dihydro-1H-inden-4-yl)amino)-4H-1,2,4-triazol-3-yl)sulfonyl)piperidin-4-amine

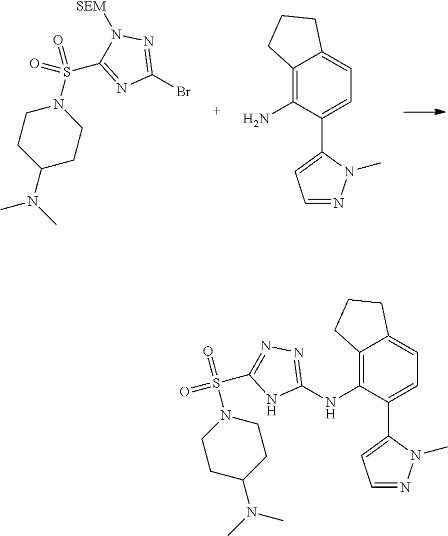 [SMILES: CN(C)C1CCN(S(=O)(=O)c2nc(Br)nn2COCC[Si](C)(C)C)CC1 ; Cn1nccc1-c1ccc2c(c1N)CCC2 ; CN(C)C1CCN(S(=O)(=O)c2nnc(Nc3c(-c4ccnn4C)ccc4c3CCC4)[nH]2)CC1]

Prepared according to the general procedure of N-(5-(2-(methoxy-d)pyridin-4-yl)-2,3-dihydrobenzofuran-4-yl)-5-((6-methyl-2,6-diazaspiro[3.4]octan-2-yl)sulfonyl)-4H-1,2,4-triazol-3-amine (Example 195) from 1-((3-bromo-1-((2-(trimethylsilyl)ethoxy)-methyl)-1H-1,2,4-triazol-5-yl)sulfonyl)-N,N-dimethylpiperidin-4-amine (Intermediate A11) and 5-(1-methyl-1H-pyrazol-5-yl)-2,3-dihydro-1H-inden-4-amine (Intermediate C17) to afford the title compound (4 mg, 6%) as a white solid.

LCMS m/z 471.4 (M+H)⁺ (ES⁺); 469.3 (M−H)⁻ (ES⁻).

¹H NMR (DMSO-d₆) δ 8.92 (s, 1H), 7.34 (d, J=1.9 Hz, 1H), 7.24 (d, J=7.7 Hz, 1H), 7.16 (d, J=7.7 Hz, 1H), 6.10 (d, J=1.9 Hz, 1H), 3.65-3.58 (m, 5H), 2.97 (t, J=7.5 Hz, 2H), 2.74 (t, J=7.4 Hz, 2H), 2.66-2.57 (m, 2H), 2.47-2.38 (m, 1H), 2.30 (s, 6H), 2.05 (p, J=7.4 Hz, 2H), 1.87-1.79 (m, 2H), 1.52-1.39 (m, 2H). One exchangeable proton not observed.

### Example 206:1-((5-((2-isopropyl-5-methylphenyl)amino)-4H-1,2,4-triazol-3-yl)sulfonyl)-N,N-dimethylpiperidin-4-amine

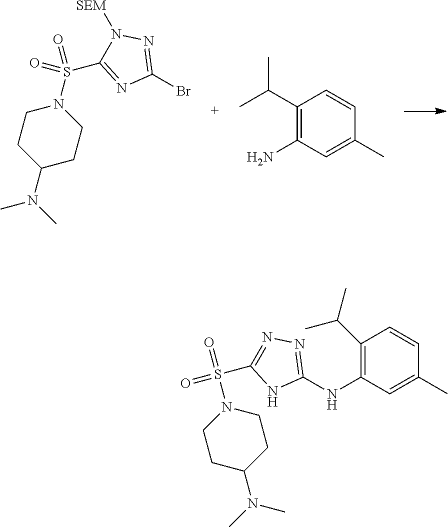 [SMILES: CN(C)C1CCN(S(=O)(=O)c2nc(Br)nn2COCC[Si](C)(C)C)CC1 ; Cc1ccc(C(C)C)c(N)c1 ; Cc1ccc(C(C)C)c(Nc2nnc(S(=O)(=O)N3CCC(N(C)C)CC3)[nH]2)c1]

Prepared according to the general procedure of N-(5-(2-(methoxy-d)pyridin-4-yl)-2,3-dihydrobenzofuran-4-yl)-5-((6-methyl-2,6-diazaspiro[3-4]octan-2-yl)sulfonyl)-4H-1,2,4-triazol-3-amine (Example 195) from 1-((3-bromo-1-((2-(trimethylsilyl)ethoxy)-methyl)-1H-1,2,4-triazol-5-yl)sulfonyl)-N,N-dimethylpiperidin-4-amine (Intermediate A11) and 2-isopropyl-5-methylaniline to afford the title compound (20 mg, 38%) as a white solid.

LCMS m/z 407.3 (M+H)⁺ (ES⁺); 405.8 (M−H)⁻ (ES⁻).

¹H NMR (DMSO-d₆) δ 8.93 (s, 1H), 7.26-7.19 (m, 2H), 6.98 (d, J=7.9 Hz, 1H), 3.86-3.73 (m, 2H), 3.16 (p, J=6.8 Hz, 1H), 3.09-3.01 (m, 1H), 2.84-2.74 (m, 2H), 2.59 (s, 6H), 2.27 (s, 3H), 2.01-1.94 (m, 2H), 1.69-1.56 (m, 2H), 1.14 (d, J=6.8 Hz, 6H). One exchangeable proton not observed.

### Example 207:N,N-dimethyl-1-((5-((5-(1-methyl-1H-pyrazol-4-yl)-2,3-dihydro-1H-inden-4-yl)amino)-4H-1,2,4-triazol-3-yl)sulfonyl)piperidin-4-amine

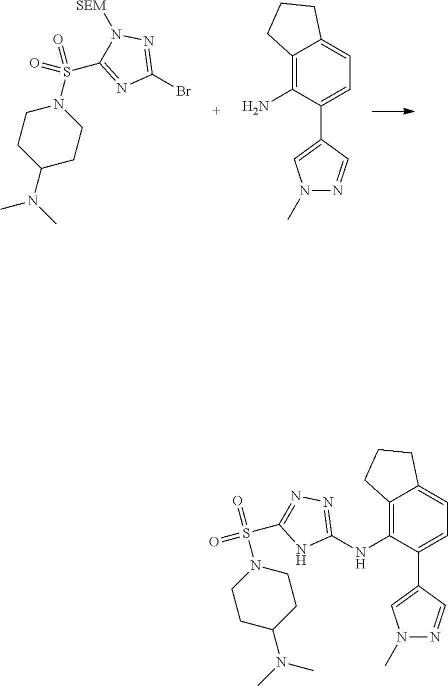 [SMILES: CN(C)C1CCN(S(=O)(=O)c2nc(Br)nn2COCC[Si](C)(C)C)CC1 ; Cn1cc(-c2ccc3c(c2N)CCC3)cn1 ; CN(C)C1CCN(S(=O)(=O)c2nnc(Nc3c(-c4cnn(C)c4)ccc4c3CCC4)[nH]2)CC1]

Prepared according to the general procedure of N-(5-(2-(methoxy-d)pyridin-4-yl)-2,3-dihydrobenzofuran-4-yl)-5-((6-methyl-2,6-diazaspiro[3.4]octan-2-yl)sulfonyl)-4H-1,2,4-triazol-3-amine (Example 195) from 1-((3-bromo-1-((2-(trimethylsilyl)ethoxy)-methyl)-1H-1,2,4-triazol-5-yl)sulfonyl)-N,N-dimethylpiperidin-4-amine (Intermediate A11) and 5-(1-methyl-H-pyrazol-4-yl)-2,3-dihydro-1H-inden-4-amine (Intermediate C6) to afford the title compound (9 mg, 7%) as a white solid.

LCMS m/z 471.3 (M+H)⁺ (ES⁺); 469.2 (M−H)⁻ (ES⁻).

¹H NMR (DMSO-d₆) δ 13.01 (s, 1H), 8.96 (s, 1H), 7.85 (s, 1H), 7.60 (s, 1H), 7.34 (d, J=7.7 Hz, 1H), 7.20 (d, J=7.8 Hz, 1H), 3.82 (s, 3H), 3.80-3.73 (m, 2H), 3.31-3.23 (m, 1H), 2.92 (t, J=7.4 Hz, 2H), 2.75 (s, 6H), 2.70 (t, J=7.5 Hz, 2H), 2.66-2.57 (m, 2H), 2.09-1.97 (m, 4H), 1.72-1.61 (m, 2H).

### Example 208: 1-((5-((2-(2-methoxypyridin-4-yl)-3-methylphenyl)amino)-4H-1,2,4-triazol-3-yl)sulfonyl)-N,N-dimethylpiperidin-4-amine

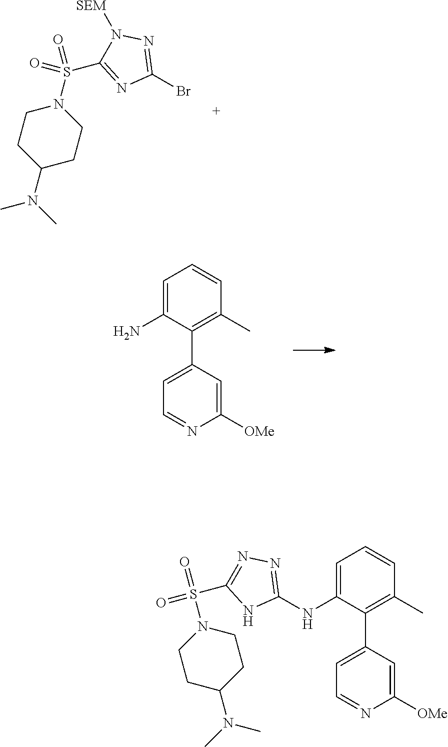 [SMILES: CN(C)C1CCN(S(=O)(=O)c2nc(Br)nn2COCC[Si](C)(C)C)CC1 ; COc1cc(-c2c(C)cccc2N)ccn1 ; COc1cc(-c2c(C)cccc2Nc2nnc(S(=O)(=O)N3CCC(N(C)C)CC3)[nH]2)ccn1]

Prepared according to the general procedure of N-(5-(2-(methoxy-d)pyridin-4-yl)-2,3-dihydrobenzofuran-4-yl)-5-((6-methyl-2,6-diazaspiro[3-4]octan-2-yl)sulfonyl)-4H-1,2,4-triazol-3-amine (Example 195) from 1-((3-bromo-1-((2-(trimethylsilyl)ethoxy)-methyl)-1H-1,2,4-triazol-5-yl)sulfonyl)-N,N-dimethylpiperidin-4-amine (Intermediate A11) and 2-(2-methoxypyridin-4-yl)-3-methylaniline (Intermediate C34) to afford the title compound (21 mg, 16%) as a white solid.

LCMS m/z 472.4 (M+H)⁺ (ES⁺); 470.3 (M−H)⁻ (ES⁻).

¹H NMR (DMSO-d₆) δ 8.24 (dd, J=5.2, 0.7 Hz, 1H), 8.20 (s, 1H), 7.68 (d, J=8.1 Hz, 1H), 7.33 (t, J=7.9 Hz, 1H), 7.09 (d, J=7.6 Hz, 1H), 6.82 (dd, J=5.2, 1.4 Hz, 1H), 6.68 (br s, 1H), 3.89 (s, 3H), 3.78-3.71 (m, 2H), 2.96-2.88 (m, 1H), 2.74-2.65 (m, 2H), 2.54 (s, 6H), 2.03 (s, 3H), 1.99-1.91 (m, 2H), 1.64-1.52 (m, 2H). One exchangeable proton not observed.

### Example 200: N,N-dimethyl-1-((5-((5-(tetrahydro-2H-pyran-4-yl)-2,3-dihydro-1H-inden-4-yl)amino)-4H-1,2,4-triazol-3-yl)sulfonyl)piperidin-4-amine

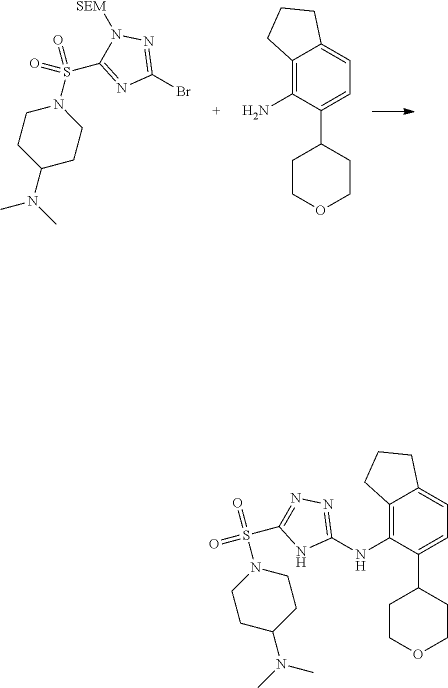 [SMILES: CN(C)C1CCN(S(=O)(=O)c2nc(Br)nn2COCC[Si](C)(C)C)CC1 ; Nc1c(C2CCOCC2)ccc2c1CCC2 ; CN(C)C1CCN(S(=O)(=O)c2nnc(Nc3c(C4CCOCC4)ccc4c3CCC4)[nH]2)CC1]

Prepared according to the general procedure of N-(5-(2-(methoxy-d)pyridin-4-yl)-2,3-dihydrobenzofuran-4-yl)-5-((6-methyl-2,6-diazaspiro[3.4]octan-2-yl)sulfonyl)-4H-1,2,4-triazol-3-amine (Example 195) from 1-((3-bromo-1-((2-(trimethylsilyl)ethoxy)-methyl)-1H-1,2,4-triazol-5-yl)sulfonyl)-N,N-dimethylpiperidin-4-amine (Intermediate A11) and 5-(tetrahydro-2H-pyran-4-yl)-2,3-dihydro-1H-inden-4-amine (Intermediate C8) to afford the title compound (12 mg, 9%) as a white solid.

LCMS m/z 475.3 (M+H)⁺ (ES⁺); 473.3 (M−H)⁻ (ES⁻).

¹H NMR (DMSO-d₆) δ 8.92 (s, 1H), 7.19-7.12 (m, 2H), 3.95-3.88 (m, 2H), 3.78-3.70 (m 2H), 3.37-3.29 (m 2H), 3.06-2.97 (m, 1H), 2.87 (t, J=7.5 Hz, 2H), 2.84-2.68 (m, 3H), 2.60 (t, J=7.4 Hz, 2H), 2.49 (s, 6H), 2.03-1.88 (m, 4H), 1.71-1.48 (m, 6H). One exchangeable proton not observed.

### Example 210: N-(5-(2-(difluoromethoxy)pyridin-4-yl)-2,3-dihydrobenzofuran-4-yl)-5-((1-methyl-1,8-diazaspiro[4.5]decan-8-yl)sulfonyl)-4H-1,2,4-triazol-3-amine

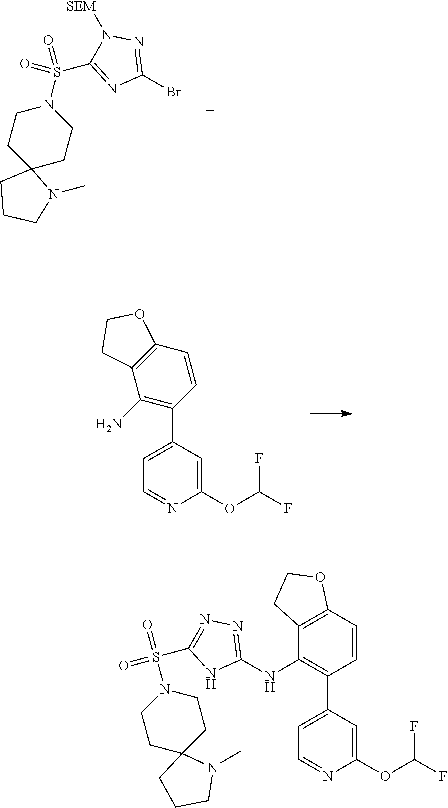 [SMILES: CN1CCCC12CCN(S(=O)(=O)c1nc(Br)nn1COCC[Si](C)(C)C)CC2 ; Nc1c(-c2ccnc(OC(F)F)c2)ccc2c1CCO2 ; CN1CCCC12CCN(S(=O)(=O)c1nnc(Nc3c(-c4ccnc(OC(F)F)c4)ccc4c3CCO4)[nH]1)CC2]

Prepared according to the general procedure of N-(5-(2-(methoxy-d)pyridin-4-yl)-2,3-dihydrobenzofuran-4-yl)-5-((6-methyl-2,6-diazaspiro[3-4]octan-2-yl)sulfonyl)-4H-1,2,4-triazol-3-amine (Example 195) from 8-((3-bromo-1-((2-(trimethylsilyl)ethoxy)-methyl)-1H-1,2,4-triazol-5-yl)sulfonyl)-1-methyl-1,8-diazaspiro[4.5]decane (Intermediate A15) and 5-(2-(difluoromethoxy)pyridin-4-yl)-2,3-dihydrobenzofuran-4-amine (Intermediate C24) to afford the title compound (30 mg, 26%) as a white solid.

LCMS m/z 562.2 (M+H)⁺ (ES⁺).

¹H NMR (DMSO-d₆) δ 9.23 (s, 1H), 8.18 (d, J=5.3 Hz, 1H), 7.68 (t, J=73.0 Hz, 1H), 7.25 (d, J=8.2 Hz, 1H), 7.18 (dd, J=5.3, 1.5 Hz, 1H), 6.96 (d, J=1.4 Hz, 1H), 6.82 (d, J=8.2 Hz, 1H), 4.61 (t, J=8.7 Hz, 2H), 3.73-3.66 (m, 2H), 3.24 (br s, 2H), 3.05 (t, J=8.7 Hz, 2H), 2.73-2.65 (m, 2H), 2.59 (s, 3H), 1.96-1.80 (m, 6H), 1.64 (br d, J=12.4 Hz, 2H). One exchangeable proton not observed.

### Example 211:N-(5-(2-methoxypyridin-4-yl)-2,3-dihydrobenzofuran-4-yl)-5-((1-methyl-1,8-diazaspiro[4.5]decan-8-yl)sulfonyl)-4H-1,2,4-triazol-3-amine

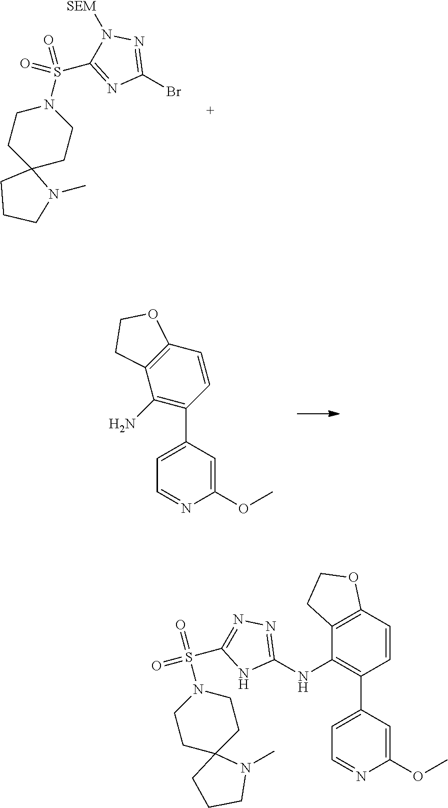 [SMILES: CN1CCCC12CCN(S(=O)(=O)c1nc(Br)nn1COCC[Si](C)(C)C)CC2 ; COc1cc(-c2ccc3c(c2N)CCO3)ccn1 ; COc1cc(-c2ccc3c(c2Nc2nnc(S(=O)(=O)N4CCC5(CCCN5C)CC4)[nH]2)CCO3)ccn1]

Prepared according to the general procedure of N-(5-(2-(methoxy-d₃)pyridin-4-yl)-2,3-dihydrobenzofuran-4-yl)-5-((6-methyl-2,6-diazaspiro[3.4]octan-2-yl)sulfonyl)-4H-1,2,4-triazol-3-amine (Example 195) from 8-((3-bromo-1-((2-(trimethylsilyl)ethoxy)-methyl)-1H-1,2,4-triazol-5-yl)sulfonyl)-1-methyl-1,8-diazaspiro[4.5]decane (Intermediate A15) and 5-(2-methoxypyridin-4-yl)-2,3-dihydrobenzofuran-4-amine (Intermediate C5) to afford the title compound (43 mg, 39%) as a white solid.

LCMS m/z 526.3 (M+H)⁺ (ES⁺).

¹H NMR (DMSO-d₆) δ 8.00 (d, J=5.4 Hz, 1H), 7.17 (d, J=8.3 Hz, 1H), 6.92 (dd, J=5.4, 1.5 Hz, 1H), 6.79 (d, J=8.3 Hz, 1H), 6.72-6.66 (m, 1H), 4.57 (t, J=8.7 Hz, 2H), 3.77 (s, 3H), 3.68 (br d, J=12.7 Hz, 2H), 3.55-3.45 (m, 1H), 3.15-2.98 (m, 3H), 2.71-2.56 (m, 5H), 2.11-1.83 (m, 4H), 1.80-1.60 (m, 4H). Two exchangeable protons not observed.

### Example 212:N-(5-(2-(difluoromethoxy)pyridin-4-yl)-2,3-dihydrobenzofuran-4-yl)-5-((6-methyl-2,6-diazaspiro[3.4]octan-2-yl)sulfonyl)-4H-1,2,4-triazol-3-amine

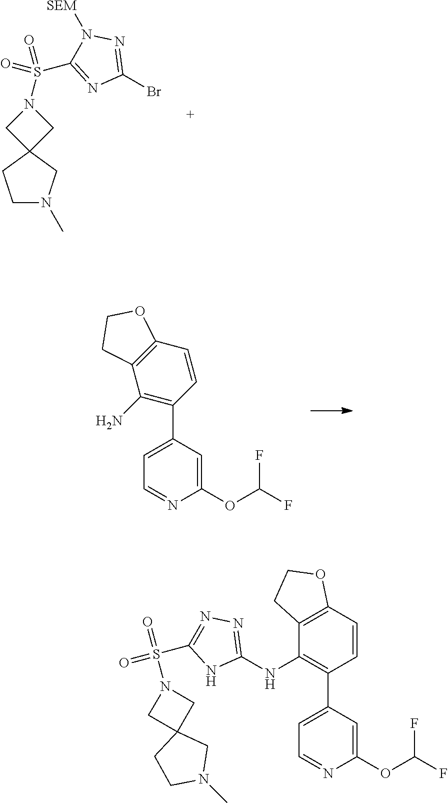 [SMILES: CN1CCC2(C1)CN(S(=O)(=O)c1nc(Br)nn1COCC[Si](C)(C)C)C2 ; Nc1c(-c2ccnc(OC(F)F)c2)ccc2c1CCO2 ; CN1CCC2(C1)CN(S(=O)(=O)c1nnc(Nc3c(-c4ccnc(OC(F)F)c4)ccc4c3CCO4)[nH]1)C2]

Prepared according to the general procedure of N-(5-(2-(methoxy-d₃)pyridin-4-yl)-2,3-dihydrobenzofuran-4-yl)-5-((6-methyl-2,6-diazaspiro[3.4]octan-2-yl)sulfonyl)-4H-1,2,4-triazol-3-amine (Example 195) from 2-((3-bromo-1-((2-(trimethylsilyl)ethoxy)-methyl)-1H-1,2,4-triazol-5-yl)sulfonyl)-6-methyl-2,6-diazaspiro[3.4]octane (Intermediate A13) and 5-(2-(difluoromethoxy)pyridin-4-yl)-2,3-dihydrobenzofuran-4-amine (Intermediate C24) to afford the title compound (28 mg, 21%) as a white solid.

LCMS m/z 534.3 (M+H)⁺ (ES⁺).

¹H NMR (DMSO-d₆) δ 9.27 (s, 1H), 8.20 (d, J=5.3 Hz, 1H), 7.70 (t, J=72.9 Hz, 1H), 7.27 (d, J=8.3 Hz, 1H), 7.20 (dd, J=5.3, 1.5 Hz, 1H), 7.00-6.99 (m, 1H), 6.85 (d, J=8.3 Hz, 1H), 4.62 (t, J=8.7 Hz, 2H), 3.95 (d, J=8.7 Hz, 2H), 3.87 (d, J=8.7 Hz, 2H), 3.25 (s, 2H), 3.15-3.11 (m, 2H), 3.05 (t, J=8.7 Hz, 2H), 2.69 (s, 3H), 2.01 (t, J=7.3 Hz, 2H). One exchangeable proton not observed.

### Example 213: N-(5-(2-(difluoromethoxy)pyridin-4-yl)-2,3-dihydro-1H-inden-4-yl)-5-((6-methyl-2,6-diazaspiro[3.4]octan-2-yl)sulfonyl)-4H-1,2,4-triazol-3-amine

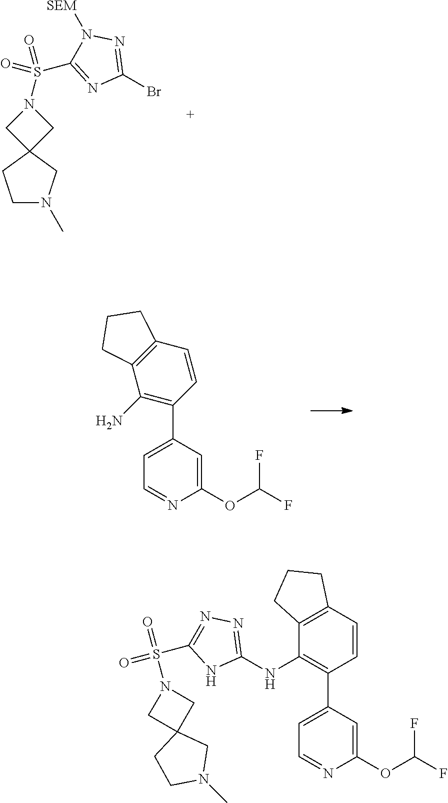 [SMILES: CN1CCC2(C1)CN(S(=O)(=O)c1nc(Br)nn1COCC[Si](C)(C)C)C2 ; Nc1c(-c2ccnc(OC(F)F)c2)ccc2c1CCC2 ; CN1CCC2(C1)CN(S(=O)(=O)c1nnc(Nc3c(-c4ccnc(OC(F)F)c4)ccc4c3CCC4)[nH]1)C2]

Prepared according to the general procedure of N-(5-(2-(methoxy-d)pyridin-4-yl)-2,3-dihydrobenzofuran-4-yl)-5-((6-methyl-2,6-diazaspiro[3-4]octan-2-yl)sulfonyl)-4H-1,2,4-triazol-3-amine (Example 195) from 2-((3-bromo-1-((2-(trimethylsilyl)ethoxy)-methyl)-1H-1,2,4-triazol-5-yl)sulfonyl)-6-methyl-2,6-diazaspiro[3.4]octane (Intermediate A13) and 5-(2-(difluoromethoxy)pyridin-4-yl)-2,3-dihydro-1H-inden-4-amine (Intermediate C8) to afford the title compound (20 mg, 15%) as a white solid.

LCMS m/z 532.3 (M+H)⁺ (ES⁺).

¹H NMR (DMSO-d₆) δ 9.12 (s, 1H), 8.23 (d, J=5.3 Hz, 1H), 7.71 (t, J=72.9 Hz, 1H), 7.32 (d, J=7.7 Hz, 1H), 7.27 (d, J=7.7 Hz, 1H), 7.22 (dd, J=5.3, 1.4 Hz, 1H), 7.02 (d, J=1.4 Hz, 1H), 3.93 (d, J=8.7 Hz, 2H), 3.85 (d, J=8.7 Hz, 2H), 3.21 (br s, 2H), 3.10 (br s, 2H), 2.98 (t, J=7.4 Hz, 2H), 2.72 (t, J=7.4 Hz, 2H), 2.67 (s, 3H), 2.05 (p, J=7.5 Hz, 2H), 1.98 (t, J=7.3 Hz, 2H). One exchangeable proton not observed.

### Example 214: N-(5-(2-methoxypyridin-4-yl)-2,3-dihydrobenzofuran-4-yl)-5-((6-methyl-2,6-diazaspiro[3.4]octan-2-yl)sulfonyl)-4H-1,2,4-triazol-3-amine

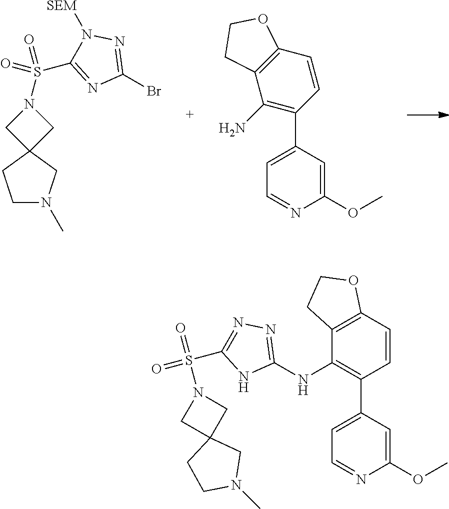 [SMILES: CN1CCC2(C1)CN(S(=O)(=O)c1nc(Br)nn1COCC[Si](C)(C)C)C2 ; COc1cc(-c2ccc3c(c2N)CCO3)ccn1 ; COc1cc(-c2ccc3c(c2Nc2nnc(S(=O)(=O)N4CC5(CCN(C)C5)C4)[nH]2)CCO3)ccn1]

Prepared according to the general procedure of N-(5-(2-(methoxy-d₃)pyridin-4-yl)-2,3-dihydrobenzofuran-4-yl)-5-((6-methyl-2,6-diazaspiro[3.4]octan-2-yl)sulfonyl)-4H-1,2,4-triazol-3-amine (Example 195) from 2-((3-bromo-1-((2-(trimethylsilyl)ethoxy)-methyl)-1H-1,2,4-triazol-5-yl)sulfonyl)-6-methyl-2,6-diazaspiro[3.4]octane (Intermediate A13) and 5-(2-methoxypyridin-4-yl)-2,3-dihydrobenzofuran-4-amine (Intermediate C5) to afford the title compound (25 mg, 21%) as a white solid.

LCMS m/z 498.3 (M+H)⁺ (ES⁺).

¹H NMR (DMSO-d₆) δ 9.20 (s, 1H), 8.10 (dd, J=5.3, 0.7 Hz, 1H), 7.21 (d, J=8.2 Hz, 1H), 6.92 (dd, J=5.3, 1.5 Hz, 1H), 6.82 (d, J=8.2 Hz, 1H), 6.73-6.72 (m, 1H), 4.61 (t, J=8.7 Hz, 2H), 3.93 (d, J=8.6 Hz, 2H), 3.85 (d, J=8.7 Hz, 2H), 3.83 (s, 3H), 3.15 (s, 2H), 3.07-3.00 (m, 4H), 2.63 (s, 3H), 1.98 (t, J=7.3 Hz, 2H). One exchangeable proton not observed.

### Example 215:N-(5-(2-methoxypyridin-4-yl)-2,3-dihydro-1H-inden-4-yl)-5-((6-methyl-2,6-diazaspiro[3.4]octan-2-yl)sulfonyl)-4H-1,2,4-triazol-3-amine

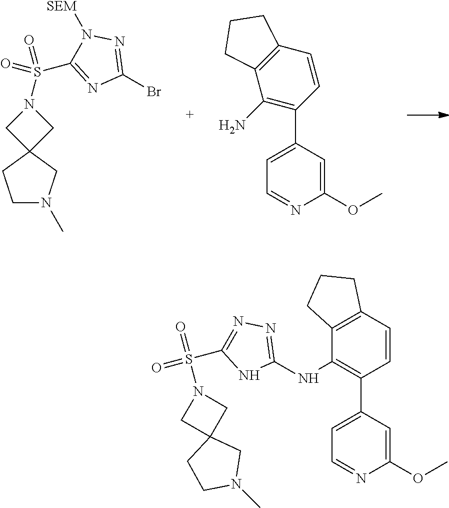 [SMILES: CN1CCC2(C1)CN(S(=O)(=O)c1nc(Br)nn1COCC[Si](C)(C)C)C2 ; COc1cc(-c2ccc3c(c2N)CCC3)ccn1 ; COc1cc(-c2ccc3c(c2Nc2nnc(S(=O)(=O)N4CC5(CCN(C)C5)C4)[nH]2)CCC3)ccn1]

Prepared according to the general procedure of N-(5-(2-(methoxy-d)pyridin-4-yl)-2,3-dihydrobenzofuran-4-yl)-5-((6-methyl-2,6-diazaspiro[3.4]octan-2-yl)sulfonyl)-4H-1,2,4-triazol-3-amine (Example 195) from 2-((3-bromo-1-((2-(trimethylsilyl)ethoxy)-methyl)-1H-1,2,4-triazol-5-yl)sulfonyl)-6-methyl-2,6-diazaspiro[3.4]octane (Intermediate A13) and 5-(2-methoxypyridin-4-yl)-2,3-dihydro-1H-inden-4-amine (Intermediate R4) to afford the title compound (20 mg, 17%) as a white solid.

LCMS m/z 496.3 (M+H)⁺ (ES⁺).

¹H NMR (DMSO-d₆) δ 9.05 (s, 1H), 8.12 (d, J=5.2 Hz, 1H), 7.29 (d, J=7.7 Hz, 1H), 7.22 (d, J=7.7 Hz, 1H), 6.94 (dd, J=5.3, 1.5 Hz, 1H), 6.76-6.75 (m, 1H), 3.88 (d, J=8.5 Hz, 2H), 3.85-3.79 (m, 5H), 3.00-2.92 (m, 4H), 2.86 (t, J=7.3 Hz, 2H), 2.71 (t, J=7.4 Hz, 2H), 2.04 (p, J=7.5 Hz, 2H), 1.91 (t, J=7.2 Hz, 2H). One exchangeable proton not observed. Three protons obscured by solvent.

### Example 216: 1-((5-((5-(2-methoxypyridin-4-yl)-6-methyl-2,3-dihydro-1H-inden-4-yl)amino)-4H-1,2,4-triazol-3-yl)sulfonyl)-N,N-dimethylpiperidin-4-amine

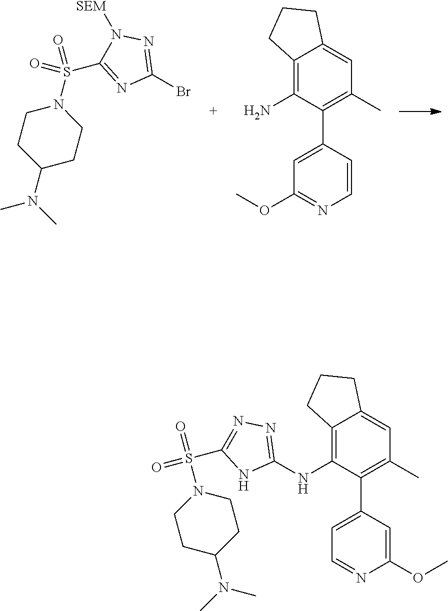

Prepared according to the general procedure of N-(5-(2-(methoxy-d)pyridin-4-yl)-2,3-dihydrobenzofuran-4-yl)-5-((6-methyl-2,6-diazaspiro[3-4]octan-2-yl)sulfonyl)-4H-1,2,4-triazol-3-amine (Example 195) from 1-((3-bromo-1-((2-(trimethylsilyl)ethoxy)-methyl)-1H-1,2,4-triazol-5-yl)sulfonyl)-N,N-dimethylpiperidin-4-amine (Intermediate A11) and 5-(2-methoxypyridin-4-yl)-6-methyl-2,3-dihydro-1H-inden-4-amine (Intermediate C20) to afford the title compound (21 mg, 17%) as a white solid.

LCMS m/z 511.9 (M+H)⁺ (ES⁺); 510.2 (M−H)⁻ (ES⁻).

¹H NMR (DMSO-d₆) δ 8.62 (s, 1H), 8.12 (d, J=5.2 Hz, 1H), 7.15 (s, 1H), 6.72 (dd, J=5.2, 1.4 Hz, 1H), 6.56 (br s, 1H), 3.83 (s, 3H), 3.70-3.61 (m, 2H), 2.92 (t, J=7.5 Hz, 2H), 2.71-2.61 (m, 3H), 2.60-2.54 (m, 2H), 2.42 (s, 6H), 2.08-1.95 (m, 5H), 1.93-1.79 (m, 2H), 1.62-1.44 (m, 2H). One exchangeable proton not observed.

### Example 217:N,N-dimethyl-1-((5-((5-(2-methylpyridin-4-yl)-2,3-dihydrobenzofuran-4-yl)amino)-4H-1,2,4-triazol-3-yl)sulfonyl)piperidin-4-amine

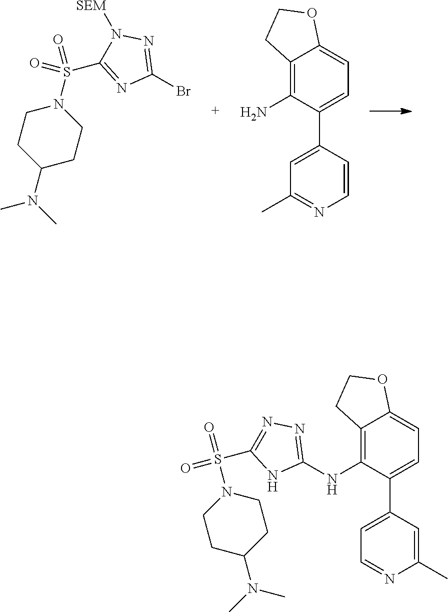 [SMILES: CN(C)C1CCN(S(=O)(=O)c2nc(Br)nn2COCC[Si](C)(C)C)CC1 ; Cc1cc(-c2ccc3c(c2N)CCO3)ccn1 ; Cc1cc(-c2ccc3c(c2Nc2nnc(S(=O)(=O)N4CCC(N(C)C)CC4)[nH]2)CCO3)ccn1]

Prepared according to the general procedure of N-(5-(2-(methoxy-d)pyridin-4-yl)-2,3-dihydrobenzofuran-4-yl)-5-((6-methyl-2,6-diazaspiro[3.4]octan-2-yl)sulfonyl)-4H-1,2,4-triazol-3-amine (Example 195) from 1-((3-bromo-1-((2-(trimethylsilyl)ethoxy)-methyl)-1H-1,2,4-triazol-5-yl)sulfonyl)-N,N-dimethylpiperidin-4-amine (Intermediate A11) and 5-(2-methylpyridin-4-yl)-2,3-dihydrobenzofuran-4-amine (Intermediate C23) to afford the title compound (3 mg, 2%) as a white solid.

LCMS m/z 484.3 (M+H)⁺ (ES⁺); 482.2 (M−H)⁻ (ES⁻).

¹H NMR (DMSO-d₆) δ 9.13 (s, 1H), 8.35 (d, J=5.2 Hz, 1H), 7.22-7.14 (m, 2H), 7.09 (dd, J=5.2, 1.7 Hz, 1H), 6.80 (d, J=8.2 Hz, 1H), 4.61 (t, J=8.7 Hz, 2H), 3.67 (brd, J=12.3 Hz, 2H), 3.06 (t, J=8.7 Hz, 2H), 2.93 (br s, 1H), 2.58 (s, 6H), 2.48-2.43 (m, 2H), 2.42 (s, 3H), 2.02-1.92 (m, 2H), 1.66-1.51 (m, 2H). One exchangeable proton not observed.

### Example 218: 1-((5-((5-(2-(methoxy-d)pyridin-4-yl)-2,3-dihydrobenzofuran-4-yl)amino)-4H-1,2,4-triazol-3-yl)sulfonyl)-N,N-dimethylpiperidin-4-amine

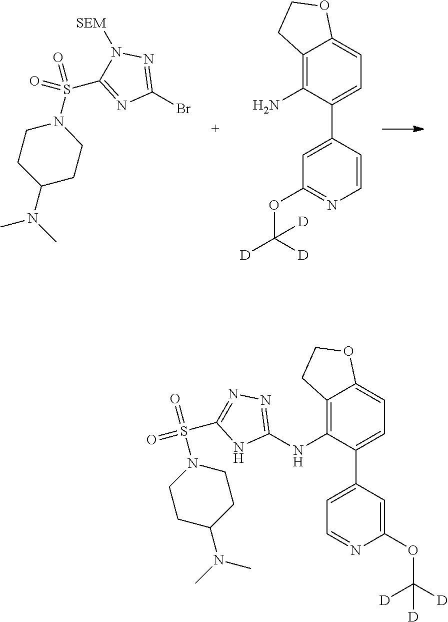 [SMILES: CN(C)C1CCN(S(=O)(=O)c2nc(Br)nn2COCC[Si](C)(C)C)CC1 ; [2H]C([2H])([2H])Oc1cc(-c2ccc3c(c2N)CCO3)ccn1 ; [2H]C([2H])([2H])Oc1cc(-c2ccc3c(c2Nc2nnc(S(=O)(=O)N4CCC(N(C)C)CC4)[nH]2)CCO3)ccn1]

Prepared according to the general procedure of N-(5-(2-(methoxy-d)pyridin-4-yl)-2,3-dihydrobenzofuran-4-yl)-5-((6-methyl-2,6-diazaspiro[3-4]octan-2-yl)sulfonyl)-4H-1,2,4-triazol-3-amine (Example 195) from 1-((3-bromo-1-((2-(trimethylsilyl)ethoxy)-methyl)-1H-1,2,4-triazol-5-yl)sulfonyl)-N,N-dimethylpiperidin-4-amine (Intermediate A11) and 5-(2-(methoxy-d₃)pyridin-4-yl)-2,3-dihydrobenzofuran-4-amine (Intermediate C22) to afford the title (74 mg, 41%) as a white solid.

LCMS m/z 503.3 (M+H)⁺ (ES⁺); 501.2 (M−H)⁻ (ES⁻).

¹H NMR (DMSO-d₆) δ 13.27 (s, 1H), 9.16 (s, 1H), 8.09 (d, J=5.4 Hz, 1H), 7.19 (d, J=8.2 Hz, 1H), 6.91 (dd, J=5-(4, 1-3 Hz, 1H), 6.80 (d, J=8.2 Hz, 1H), 6.74-6.69 (m, 1H), 4.60 (t, J=8.7 Hz, 2H), 3.76-3.71 (m, 2H), 3.29-3.21 (m, 1H), 3.05 (t, J=8.7 Hz, 2H), 2.75 (s, 6H), 2.60-2.51 (m, 2H), 2.10-2.01 (m, 2H), 1.76-1.61 (m, 2H).

### Example 219: N,N-dimethyl-1-((5-((5-(pyridin-4-yl)-2,3-dihydro-1H-inden-4-yl)amino)-4H-1,2,4-triazol-3-yl)sulfonyl)piperidin-4-amine

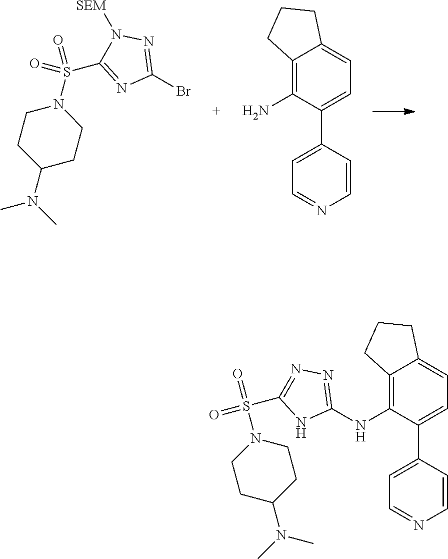 [SMILES: CN(C)C1CCN(S(=O)(=O)c2nc(Br)nn2COCC[Si](C)(C)C)CC1 ; Nc1c(-c2ccncc2)ccc2c1CCC2 ; CN(C)C1CCN(S(=O)(=O)c2nnc(Nc3c(-c4ccncc4)ccc4c3CCC4)[nH]2)CC1]

Prepared according to the general procedure of N-(5-(2-(methoxy-d)pyridin-4-yl)-2,3-dihydrobenzofuran-4-yl)-5-((6-methyl-2,6-diazaspiro[3.4]octan-2-yl)sulfonyl)-4H-1,2,4-triazol-3-amine (Example 195) from 1-((3-bromo-1-((2-(trimethylsilyl)ethoxy)-methyl)-1H-1,2,4-triazol-5-yl)sulfonyl)-N,N-dimethylpiperidin-4-amine (Intermediate A11) and 5-(pyridin-4-yl)-2,3-dihydro-1H-inden-4-amine (Intermediate C4) to afford the title compound (13 mg, 8%) as a white solid.

LCMS m/z 468.4 (M+H)⁺ (ES⁺); 466.2 (M−H)⁻ (ES⁻).

¹H NMR (DMSO-d₆) δ 12.92 (s, 1H), 8.99 (s, 1H), 8.58-8.42 (m, 2H), 7.37-7.31 (m, 2H), 7.28 (d, J=7.7 Hz, 1H), 7.22 (d, J=7.6 Hz, 1H), 3.66-3.47 (m 2H), 2.96 (t, J=7.4 Hz, 2H), 2.75 (t, J=7.4 Hz, 2H), 2.18 (s, 6H), 2.09-1.95 (m, 2H), 1.83-1.67 (m, 2H), 1.50-1.32 (m, 2H). Three protons obscured by solvent.

### Example 220: N,N-dimethyl-1-((5-((5-(pyridin-4-yl)-2,3-dihydrobenzofuran-4-yl)amino)-4H-1,2,4-triazol-3-yl)sulfonyl)piperidin-4-amine

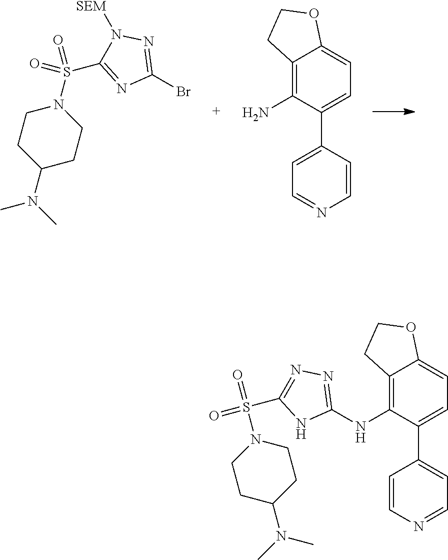 [SMILES: CN(C)C1CCN(S(=O)(=O)c2nc(Br)nn2COCC[Si](C)(C)C)CC1 ; Nc1c(-c2ccncc2)ccc2c1CCO2 ; CN(C)C1CCN(S(=O)(=O)c2nnc(Nc3c(-c4ccncc4)ccc4c3CCO4)[nH]2)CC1]

Prepared according to the general procedure of N-(5-(2-(methoxy-d)pyridin-4-yl)-2,3-dihydrobenzofuran-4-yl)-5-((6-methyl-2,6-diazaspiro[3.4]octan-2-yl)sulfonyl)-4H-1,2,4-triazol-3-amine (Example 195) from 1-((3-bromo-1-((2-(trimethylsilyl)ethoxy)-methyl)-1H-1,2,4-triazol-5-yl)sulfonyl)-N,N-dimethylpiperidin-4-amine (Intermediate A11) and 5-(pyridin-4-yl)-2,3-dihydrobenzofuran-4-amine (Intermediate C25) to afford the title compound (31 mg, 21%) as a white solid.

LCMS m/z 470.4 (M+H)⁺ (ES⁺); 468.1 (M−H)⁻ (ES⁻).

¹H NMR (DMSO-d₆) δ 12.97 (s, 1H), 9.09 (s, 1H), 8.54-8.39 (m, 2H), 7.34-7.29 (m, 2H), 7.20 (d, J=8.2 Hz, 1H), 6.81 (d, J=8.2 Hz, 1H), 4.60 (t, J=8.7 Hz, 2H), 3.63-3.46 (m, 2H), 3.06 (t, J=8.7 Hz, 2H), 2.17 (s, 6H), 1.82-1.67 (m, 2H), 1.45-1.30 (m, 2H). Three protons obscured by solvent.

### Example 221: N,N-dimethyl-1-((5-((5-(2-methylpyridin-4-yl)-2,3-dihydro-1H-inden-4-yl)amino)-4H-1,2,4-triazol-3-yl)sulfonyl)piperidin-4-amine

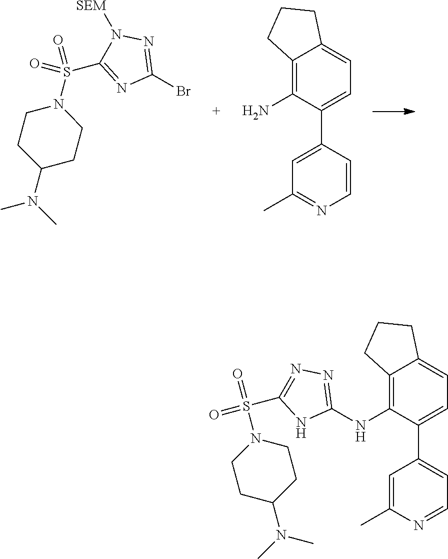 [SMILES: CN(C)C1CCN(S(=O)(=O)c2nc(Br)nn2COCC[Si](C)(C)C)CC1 ; Cc1cc(-c2ccc3c(c2N)CCC3)ccn1 ; Cc1cc(-c2ccc3c(c2Nc2nnc(S(=O)(=O)N4CCC(N(C)C)CC4)[nH]2)CCC3)ccn1]

Prepared according to the general procedure of N-(5-(2-(methoxy-d)pyridin-4-yl)-2,3-dihydrobenzofuran-4-yl)-5-((6-methyl-2,6-diazaspiro[3.4]octan-2-yl)sulfonyl)-4H-1,2,4-triazol-3-amine (Example 195) from 1-((3-bromo-1-((2-(trimethylsilyl)ethoxy)-methyl)-1H-1,2,4-triazol-5-yl)sulfonyl)-N,N-dimethylpiperidin-4-amine (Intermediate A11) and 5-(2-methylpyridin-4-yl)-2,3-dihydro-1H-inden-4-amine (Intermediate C7) to afford the title compound (24 mg, 15%) as a white solid.

LCMS m/z 482.4 (M+H)⁺ (ES⁺); 480.2 (M−H)⁻ (ES⁻).

¹H NMR (DMSO-d₆) δ 12.91 (s, 1H), 8.96 (s, 1H), 8.36 (dd, J=5.1, 0.8 Hz, 1H), 7.27 (d, J=7.7 Hz, 1H), 7.22-7.16 (m, 2H), 7.11 (dd, J=5.1, 1.6 Hz, 1H), 3.59-3.52 (m, 2H), 2.96 (t, J=7.4 Hz, 2H), 2.74 (t, J=7.4 Hz, 2H), 2.42 (s, 3H), 2.18 (s, 6H), 2.07-2.00 (m, 2H), 1.80-1.71 (m, 2H), 1.47-1.33 (m, 2H). Three protons obscured by solvent.

### Example 222: 1-((5-((5-(2-(difluoromethoxy)pyridin-4-yl)-2,3-dihydro-1H-inden-4-yl)amino)-4H-1,2,4-triazol-3-yl)sulfonyl)-N,N-dimethylpiperidin-4-amine

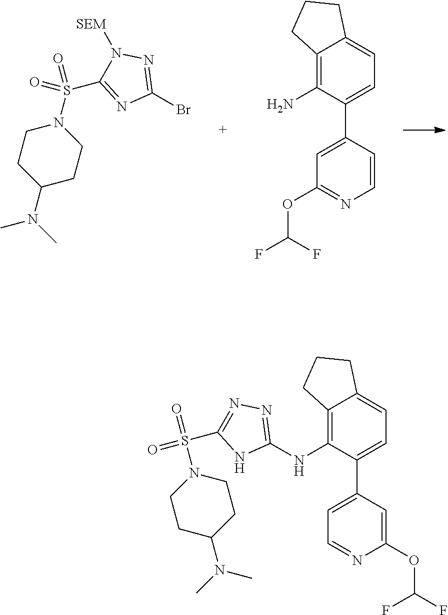 [SMILES: CN(C)C1CCN(S(=O)(=O)c2nc(Br)nn2COCC[Si](C)(C)C)CC1 ; Nc1c(-c2ccnc(OC(F)F)c2)ccc2c1CCC2 ; CN(C)C1CCN(S(=O)(=O)c2nnc(Nc3c(-c4ccnc(OC(F)F)c4)ccc4c3CCC4)[nH]2)CC1]

Prepared according to the general procedure of N-(5-(2-(methoxy-d)pyridin-4-yl)-2,3-dihydrobenzofuran-4-yl)-5-((6-methyl-2,6-diazaspiro[3.4]octan-2-yl)sulfonyl)-4H-1,2,4-triazol-3-amine (Example 195) from 1-((3-bromo-1-((2-(trimethylsilyl)ethoxy)-methyl)-1H-1,2,4-triazol-5-yl)sulfonyl)-N,N-dimethylpiperidin-4-amine (Intermediate A11) and 5-(2-(difluoromethoxy)pyridin-4-yl)-2,3-dihydro-1H-inden-4-amine (Intermediate C8) to afford the title compound (32 mg, 16%) as a white solid.

LCMS m/z 534.2 (M+H)⁺ (ES⁺); 532.1 (M−H)⁻ (ES⁻). 20 ¹H NMR (DMSO-d₆) δ 9.05 (s, 1H), 8.21 (d, J=5.3 Hz, 1H), 7.69 (t, J=72.9 Hz, 1H), 7.30 (d, J=7.7 Hz, 1H), 7.26 (d, J=7.7 Hz, 1H), 7.21 (dd, J=5.3, 1.5 Hz, 1H), 7.00 (br s, 1H), 3.68 (br d, J=12.3 Hz, 2H), 2.97 (t, J=7.4 Hz, 2H), 2.86-2.78 (m, 1H), 2.74 (t, J=7.4 Hz, 2H), 2.59-2.51 (m, 8H), 2.04 (p, J=7.5 Hz, 2H), 1.98-1.87 (m, 2H), 1.64-1.47 (m, 2H). One exchangeable proton not observed.

### Example 223: 8-((5-((4-(dimethylamino)piperidin-1-yl)sulfonyl)-4H-1,2,4-triazol-3-yl)amino)-1,2,3,5,6,7-hexahydro-s-indacene-4-carbonitrile

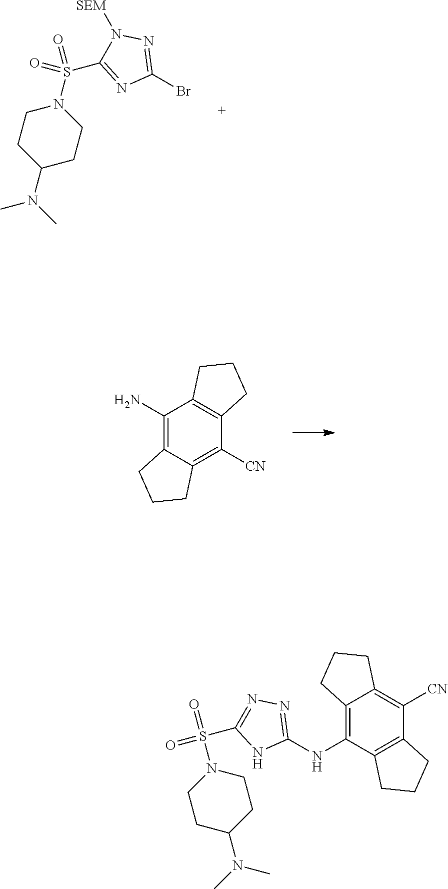 [SMILES: CN(C)C1CCN(S(=O)(=O)c2nc(Br)nn2COCC[Si](C)(C)C)CC1 ; N#Cc1c2c(c(N)c3c1CCC3)CCC2 ; CN(C)C1CCN(S(=O)(=O)c2nnc(Nc3c4c(c(C#N)c5c3CCC5)CCC4)[nH]2)CC1]

Prepared according to the general procedure of N-(5-(2-(methoxy-d)pyridin-4-yl)-2,3-dihydrobenzofuran-4-yl)-5-((6-methyl-2,6-diazaspiro[3.4]octan-2-yl)sulfonyl)-4H-1,2,4-triazol-3-amine (Example 195) from 1-((3-bromo-1-((2-(trimethylsilyl)ethoxy)-methyl)-1H-1,2,4-triazol-5-yl)sulfonyl)-N,N-dimethylpiperidin-4-amine (Intermediate A11) and 8-amino-1,2,3,5,6,7-hexahydro-s-indacene-4-carbonitrile (Intermediate C11) to afford the title compound (70 mg, 47%) as a white solid.

LCMS m/z 456.3 (M+H)⁺ (ES⁺); 454.2 (M−H)⁻ (ES⁻).

¹H NMR (DMSO-d₆) δ 9.41 (s, 1H), 3.86-3.68 (m, 2H), 3.15-3.04 (m, 1H), 2.98 (t, J=7.4 Hz, 4H), 2.79-2.72 (m, 2H), 2.67 (t, J=7.4 Hz, 4H), 2.62 (s, 6H), 2.06 (p, J=7.5 Hz, 4H), 2.02-1.97 (m, 2H), 1.62 (app. qd, J=12.2, 4.3 Hz, 2H). One exchangeable proton not observed.

### Example 224:1-((5-((8-fluoro-1,2,3,5,6,7-hexahydro-s-indacen-4-yl)amino)-4H-1,2,4-triazol-3-yl)sulfonyl)-N,N-dimethylpiperidin-4-amine

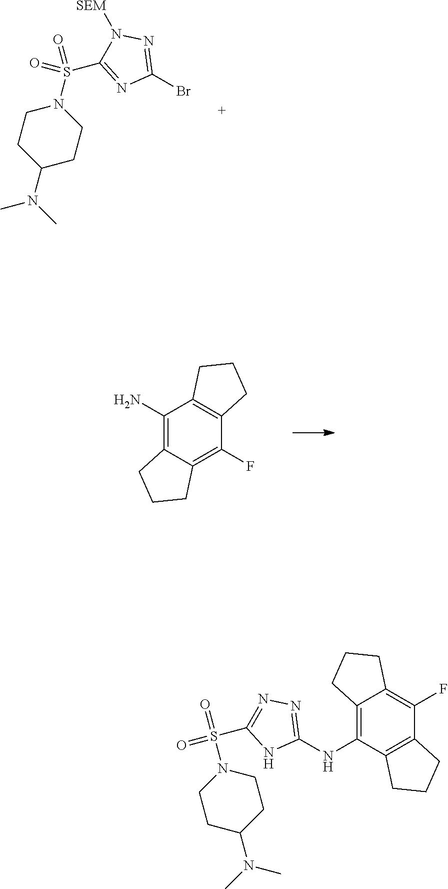 [SMILES: CN(C)C1CCN(S(=O)(=O)c2nc(Br)nn2COCC[Si](C)(C)C)CC1 ; Nc1c2c(c(F)c3c1CCC3)CCC2 ; CN(C)C1CCN(S(=O)(=O)c2nnc(Nc3c4c(c(F)c5c3CCC5)CCC4)[nH]2)CC1]

Prepared according to the general procedure of N-(5-(2-(methoxy-d)pyridin-4-yl)-2,3-dihydrobenzofuran-4-yl)-5-((6-methyl-2,6-diazaspiro[3.4]octan-2-yl)sulfonyl)-4H-1,2,4-triazol-3-amine (Example 195) from 1-((3-bromo-1-((2-(trimethylsilyl)ethoxy)-methyl)-1H-1,2,4-triazol-5-yl)sulfonyl)-N,N-dimethylpiperidin-4-amine (Intermediate A11) and 8-fluoro-1,2,3,5,6,7-hexahydro-s-indacen-4-amine (Intermediate C12) to afford the title compound (46 mg, 32%) as a white solid.

LCMS m/z 449.2 (M+H)⁺ (ES⁺); 447.1 (M−H)⁻ (ES⁻).

¹H NMR (DMSO-d₆) δ 8.98 (s, 1H), 3.77 (br d, J=12.5 Hz, 2H), 3.13-3.01 (m, 1H), 2.86 (t, J=7.4 Hz, 4H), 2.80-2.72 (m 2H), 2.68 (t, J=7.4 Hz, 4H), 2.61 (s, 6H), 2.22 -1.89 (m, 6H), 1.61 (app. qd, J=12.4, 4.3 Hz, 2H). One exchangeable proton not observed.

### Example 22S: 1-((5-((5-(2-(difluoromethoxy)pyridin-4-yl)-2,3-dihydrobenzofuran-4-yl)amino)-4H-1,2,4-triazol-3-yl)sulfonyl)-N,N-dimethylpiperidin-4-amine

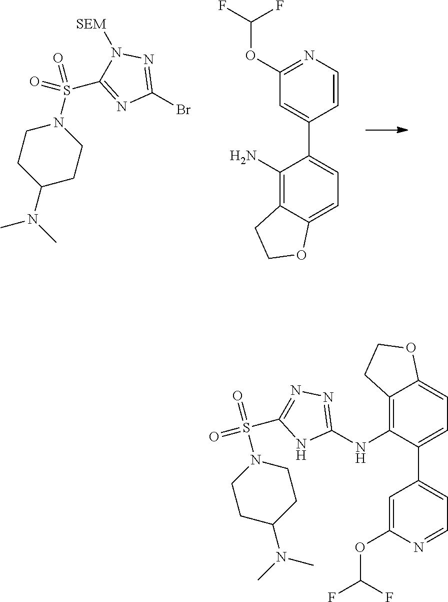 [SMILES: CN(C)C1CCN(S(=O)(=O)c2nc(Br)nn2COCC[Si](C)(C)C)CC1 ; Nc1c(-c2ccnc(OC(F)F)c2)ccc2c1CCO2 ; CN(C)C1CCN(S(=O)(=O)c2nnc(Nc3c(-c4ccnc(OC(F)F)c4)ccc4c3CCO4)[nH]2)CC1]

Prepared according to the general procedure of N-(5-(2-(methoxy-d)pyridin-4-yl)-2,3-dihydrobenzofuran-4-yl)-5-((6-methyl-2,6-diazaspiro[3.4]octan-2-yl)sulfonyl)-4H-1,2,4-triazol-3-amine (Example 195) from 1-((3-bromo-1-((2-(trimethylsilyl)ethoxy)-methyl)-1H-1,2,4-triazol-5-yl)sulfonyl)-N,N-dimethylpiperidin-4-amine (Intermediate A11) and 5-(2-(difluoromethoxy)pyridin-4-yl)-2,3-dihydrobenzofuran-4-amine (Intermediate C24) to afford the title compound (52 mg, 30%) as a white solid.

LCMS m/z 536.2 (M+H)⁺ (ES⁺).

¹H NMR (DMSO-d₆) δ 9.27 (s, 1H), 8.24-8.12 (m, 2H), 7.68 (t, J=73.0 Hz, 1H), 7.25 (d, J=8.3 Hz, 1H), 7.20 (dd, J=5.3, 1.5 Hz, 1H), 6.97 (d, J=1.3 Hz, 1H), 6.82 (d, J=8.3 Hz, 1H), 4.62 (t, J=8.7 Hz, 2H), 3.59 (br d, J=12.4 Hz, 2H), 3.07 (t, J=8.7 Hz, 2H), 2.22 (s, 6H), 1.85-1.73 (m, 2H), 1.52-1.32 (m, 2H). Three protons obscured by solvent.

### Example 226: 1-((5-((5-(2-methoxypyridin-4-yl)-2,3-dihydrobenzofuran-4-yl)amino)-4H-1,2,4-triazol-3-yl)sulfonyl)-N,N-dimethylpiperidin-4-amine

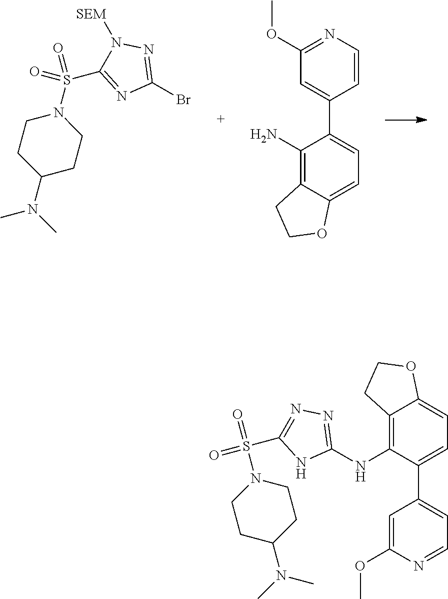 [SMILES: CN(C)C1CCN(S(=O)(=O)c2nc(Br)nn2COCC[Si](C)(C)C)CC1 ; COc1cc(-c2ccc3c(c2N)CCO3)ccn1 ; COc1cc(-c2ccc3c(c2Nc2nnc(S(=O)(=O)N4CCC(N(C)C)CC4)[nH]2)CCO3)ccn1]

Prepared according to the general procedure of N-(5-(2-(methoxy-d₃)pyridin-4-yl)-2,3-dihydrobenzofuran-4-yl)-5-((6-methyl-2,6-diazaspiro[3.4]octan-2-yl)sulfonyl)-4H-1,2,4-triazol-3-amine (Example 195) from 1-((3-bromo-1-((2-(trimethylsilyl)ethoxy)-methyl)-1H-1,2,4-triazol-5-yl)sulfonyl)-N,N-dimethylpiperidin-4-amine (Intermediate A11) and 5-(2-methoxypyridin-4-yl)-2,3-dihydrobenzofuran-4-amine (Intermediate C5) to afford the title compound (60 mg, 37%) as a white solid.

LCMS m/z 500.3 (M+H)⁺ (ES⁺).

¹H NMR (DMSO-d₆) δ 13.04 (s, 1H), 9.06 (s, 1H), 8.07 (d, J=5.3 Hz, 1H), 7.19 (d, J=8.2 Hz, 1H), 6.92 (dd, J=5.3, 1.5 Hz, 1H), 6.79 (d, J=8.2 Hz, 1H), 6.73-6.69 (m, 1H), 4.60 (t, J=8.7 Hz, 2H), 3.82 (s, 3H), 3.57 (br d, J=12.4 Hz, 2H), 3.17 (br s, 2H), 3.06 (t, J=8.7 Hz, 2H), 2.17 (s, 6H), 1.86-1.71 (m, 2H), 1.53-1.31 (m, 2H). One proton obscured by solvent.

### Example 227: 1-((5-((5-(2-(methoxy-d₃)pyridin-4-yl)-2,3-dihydro-1H-inden-yl)amino)-4H-1,2,4-triazol-3-yl)sulfonyl)-N,N-dimethylpiperidin-4-amine

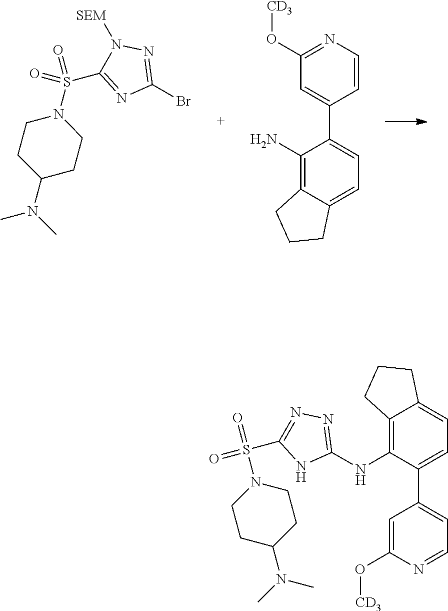 [SMILES: CN(C)C1CCN(S(=O)(=O)c2nc(Br)nn2COCC[Si](C)(C)C)CC1 ; [2H]C([2H])([2H])Oc1cc(-c2ccc3c(c2N)CCC3)ccn1 ; [2H]C([2H])([2H])Oc1cc(-c2ccc3c(c2Nc2nnc(S(=O)(=O)N4CCC(N(C)C)CC4)[nH]2)CCC3)ccn1]

Prepared according to the general procedure of N-(5-(2-(methoxy-d)pyridin-4-yl)-2,3-dihydrobenzofuran-4-yl)-5-((6-methyl-2,6-diazaspiro[3.4]octan-2-yl)sulfonyl)-4H-1,2,4-triazol-3-amine (Example 195) from 1-((3-bromo-1-((2-(trimethylsilyl)ethoxy)-methyl)-1H-1,2,4-triazol-5-yl)sulfonyl)-N,N-dimethylpiperidin-4-amine (Intermediate A11) and 5-(2-(methoxy-d₃)pyridin-4-yl)-2,3-dihydro-1H-inden-4-amine (Intermediate C21) to afford the title compound (27 mg, 17%) as a white solid.

LCMS m/z 501.4 (M+H)⁺ (ES⁺).

¹H NMR (DMSO-d₆) δ 9.01 (s, 1H), 8.17 (s, 1H), 8.09 (d, J=5.3 Hz, 1H), 7.26 (d, J=7.7 Hz, 1H), 7.20 (d, J=7.6 Hz, 1H), 6.93 (dd, J=5.3, 1.1 Hz, 1H), 6.74 (d, J=1.2 Hz, 1H), 3.58 (br d, J=12.2 Hz, 2H), 2.96 (t, J=7.4 Hz, 2H), 2.74 (t, J=7.4 Hz, 2H), 2.23 (s, 6H), 2.03 (p, J=7.5 Hz, 2H), 1.80 (br d, J=12.6 Hz, 2H), 1.59-1.32 (m, 2H). Three protons obscured by solvent.

### Example 228: 1-((5-((5-(2-methoxypyridin-4-yl)-2,3-dihydro-1H-inden-4-yl)amino)-4H-1,2,4-triazol-3-yl)sulfonyl)-N,N-dimethylpiperidin-4-amine

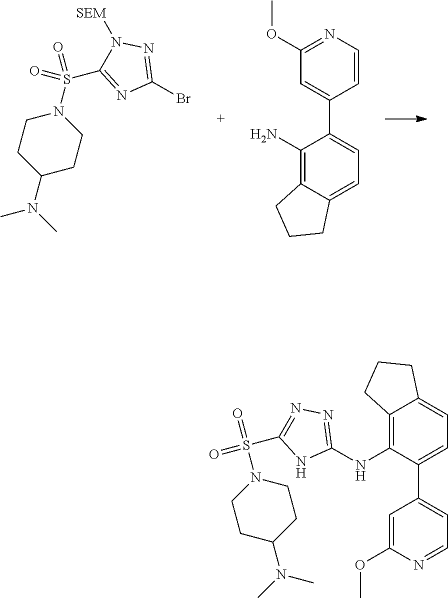 [SMILES: CN(C)C1CCN(S(=O)(=O)c2nc(Br)nn2COCC[Si](C)(C)C)CC1 ; COc1cc(-c2ccc3c(c2N)CCC3)ccn1 ; COc1cc(-c2ccc3c(c2Nc2nnc(S(=O)(=O)N4CCC(N(C)C)CC4)[nH]2)CCC3)ccn1]

Prepared according to the general procedure of N-(5-(2-(methoxy-d)pyridin-4-yl)-2,3-dihydrobenzofuran-4-yl)-5-((6-methyl-2,6-diazaspiro[3-4]octan-2-yl)sulfonyl)-4H-1,2,4-triazol-3-amine (Example 195) from 1-((3-bromo-1-((2-(trimethylsilyl)ethoxy)-methyl)-1H-1,2,4-triazol-5-yl)sulfonyl)-N,N-dimethylpiperidin-4-amine (Intermediate A11) and 5-(2-methoxypyridin-4-yl)-2,3-dihydro-1H-inden-4-amine (Intermediate R4) to afford the title compound (7 mg, 14%) as a white solid.

LCMS m/z 498.3 (M+H)⁺ (ES⁺); 496.2 (M−H)⁻ (ES⁻).

¹H NMR (DMSO-d₆) δ 9.02 (s, 1H), 8.18 (s, 1H), 8.09 (d, J=5.3 Hz, 1H), 7.26 (d, J=7.7 Hz, 1H), 7.20 (d, J=7.7 Hz, 1H), 6.93 (dd, J=5.3, 1.4 Hz, 1H), 6.74 (d, J=1.5 Hz, 1H), 3.82 (s, 3H), 3.57 (brd, J=12.3 Hz, 2H), 2.96 (t, J=7.4 Hz, 2H), 2.74 (t, J=7.4 Hz, 2H), 2.20 (s, 6H), 2.03 (p, J=7.6 Hz, 2H), 1.78 (br d, J=12.1 Hz, 2H), 1.40 (app. qd, J=12.0, 4.1 Hz, 2H). Three protons obscured by solvent.

### Example 229:1-((5-((5-(2-methoxypyridin-4-yl)-7-(oxazol-2-yl)-2,3-dihydro-1H-inden-4-yl)amino)-4H-1,2,4-triazol-3-yl)sulfonyl)-N,N-dimethylpiperidin-4-amine

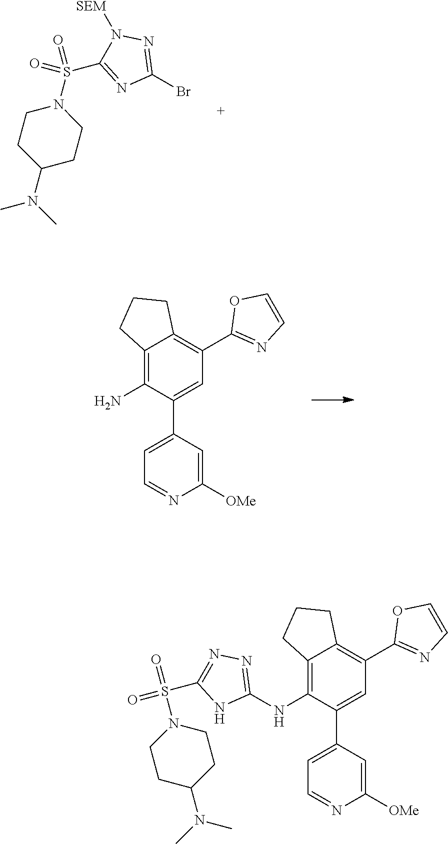 [SMILES: CN(C)C1CCN(S(=O)(=O)c2nc(Br)nn2COCC[Si](C)(C)C)CC1 ; COc1cc(-c2cc(-c3ncco3)c3c(c2N)CCC3)ccn1 ; COc1cc(-c2cc(-c3ncco3)c3c(c2Nc2nnc(S(=O)(=O)N4CCC(N(C)C)CC4)[nH]2)CCC3)ccn1]

Prepared according to the general procedure of N-(5-(2-(methoxy-d)pyridin-4-yl)-2,3-dihydrobenzofuran-4-yl)-5-((6-methyl-2,6-diazaspiro[3.4]octan-2-yl)sulfonyl)-4H-1,2,4-triazol-3-amine (Example 195) from 1-((3-bromo-1-((2-(trimethylsilyl)ethoxy)-methyl)-1H-1,2,4-triazol-5-yl)sulfonyl)-N,N-dimethylpiperidin-4-amine (Intermediate A11) and 5-(2-methoxypyridin-4-yl)-7-(oxazol-2-yl)-2,3-dihydro-1H-inden-4-amine (Intermediate C13) to afford the title compound (26 mg, 18%) as a white solid.

LCMS m/z 565.3 (M+H)⁺ (ES⁺); 563.2 (M−H)⁻ (ES⁻).

¹H NMR (DMSO-d₆) δ 9.22 (s, 1H), 8.25 (d, J=0.8 Hz, 1H), 8.16 (d, J=5.1 Hz, 1H), 7.79 (s, 1H), 7.44 (d, J=0.8 Hz, 1H), 7.00 (dd, J=5.3, 1.5 Hz, 1H), 6.83-6.79 (m, 1H), 3.85 (s, 3H), 3.72-3.65 (m, 2H), 3.37 (t, J=7.5 Hz, 2H), 2.99-2.88 (m, 1H), 2.83 (t, J=7.5 Hz, 2H), 2.58 (s, 6H), 2.12 (p, J=7.5 Hz, 2H), 2.01-1.92 (m, 2H), 1.65-1.51 (m, 2H).

One exchangeable proton not observed. Two protons obscured by solvent.

### Example 230:7-((5-((4-(dimethylamino)piperidin-1-yl)sulfonyl)-4H-1,2,4-triazol-3-yl)amino)-6-(2-methoxypyridin-4-yl)-2,3-dihydro-1H-indene-4-carbonitrile

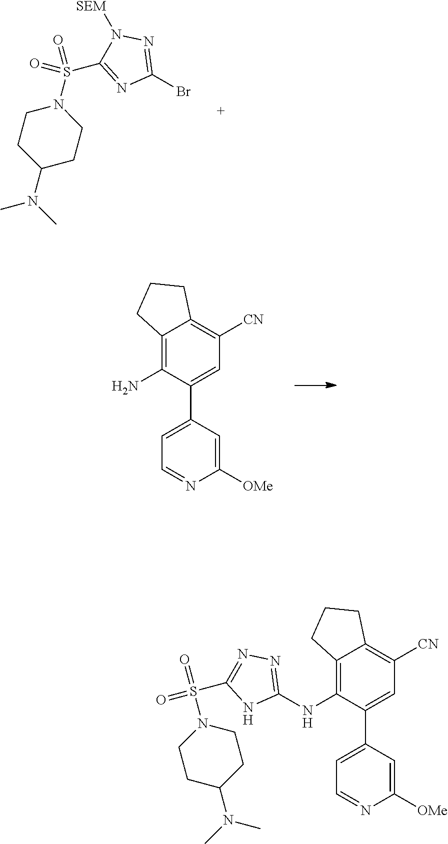 [SMILES: CN(C)C1CCN(S(=O)(=O)c2nc(Br)nn2COCC[Si](C)(C)C)CC1 ; COc1cc(-c2cc(C#N)c3c(c2N)CCC3)ccn1 ; COc1cc(-c2cc(C#N)c3c(c2Nc2nnc(S(=O)(=O)N4CCC(N(C)C)CC4)[nH]2)CCC3)ccn1]

Prepared according to the general procedure of N-(5-(2-(methoxy-d)pyridin-4-yl)-2,3-dihydrobenzofuran-4-yl)-5-((6-methyl-2,6-diazaspiro[3.4]octan-2-yl)sulfonyl)-4H-1,2,4-triazol-3-amine (Example 195) from 1-((3-bromo-1-((2-(trimethylsilyl)ethoxy)-methyl)-1H-1,2,4-triazol-5-yl)sulfonyl)-N,N-dimethylpiperidin-4-amine (Intermediate A11) and 7-amino-6-(2-methoxypyridin-4-yl)-2,3-dihydro-1H-indene-4-carbonitrile (Intermediate C6) to afford the title compound (21 mg, 15%) as a white solid.

LCMS m/z 523.3 (M+H)⁺ (ES⁺); 521.3 (M−H)⁻ (ES⁻).

¹H NMR (DMSO-d₆) δ 9.37 (s, 1H), 8.12 (d, J=5.3 Hz, 1H), 7.67 (s, 1H), 6.94 (dd, J=5.3, 1.5 Hz, 1H), 6.80-6.77 (m, 1H), 3.84 (s, 3H), 3.68-3.60 (m, 2H), 3.11 (t, J=7.5 Hz, 2H), 2.81 (t, J=7.5 Hz, 2H), 2.73-2.64 (m, 1H), 2.46 (s, 6H), 2.13 (p, J=7.5 Hz, 2H), 1.95-1.86 (m, 2H), 1.58-1.46 (m, 2H). One exchangeable proton not observed. Two protons obscured by solvent.

### Example 231: (S)-2-(1-((5-((5-(2-methoxypyridin-4-yl)-2,3-dihydro-1H-inden-4-yl)amino)-4H-1,2,4-triazol-3-yl)sulfonyl)pyrrolidin-3-yl)propan-2-ol

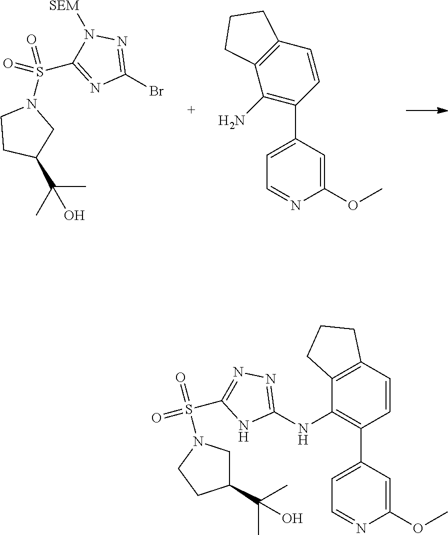 [SMILES: CC(C)(O)[C@H]1CCN(S(=O)(=O)c2nc(Br)nn2COCC[Si](C)(C)C)C1 ; COc1cc(-c2ccc3c(c2N)CCC3)ccn1 ; COc1cc(-c2ccc3c(c2Nc2nnc(S(=O)(=O)N4CC[C@H](C(C)(C)O)C4)[nH]2)CCC3)ccn1]

Prepared according to the general procedure of N-(5-(2-(methoxy-d)pyridin-4-yl)-2,3-dihydrobenzofuran-4-yl)-5-((6-methyl-2,6-diazaspiro[3-4]octan-2-yl)sulfonyl)-4H-1,2,4-triazol-3-amine (Example 195) from (S)-2-(1-((3-bromo-1-((2-(trimethylsilyl)-ethoxy)methyl)-1H-1,2,4-triazol-5-yl)sulfonyl)pyrrolidin-3-yl)propan-2-ol (Intermediate A10) and 5-(2-methoxypyridin-4-yl)-2,3-dihydro-1H-inden-4-amine (Intermediate R4) to afford the title compound (7 mg, 6%) as a white solid.

LCMS m/z 499.3 (M+H)⁺ (ES⁺); 497.2 (M−H)⁻ (ES⁻).

¹H NMR (DMSO-d₆) δ 13.10 (s, 1H), 8.96 (s, 1H), 8.10 (dd, J=5.3, 0.7 Hz, 1H), 7.26 (d, J=7.7 Hz, 1H), 7.20 (d, J=7.6 Hz, 1H), 6.91 (dd, J=5.3, 1.5 Hz, 1H), 6.73 (dd, J=1.5, 0.7 Hz, 1H), 4.35 (s, 1H), 3.83 (s, 3H), 3.39-3.32 (m, 1H), 3.31-3.26 (m, 1H), 3.15-3.04 (m, 2H), 2.95 (t, J=7.4 Hz, 2H), 2.69 (t, J=7.4 Hz, 2H), 2.07-1.95 (m, 3H), 1.78-1.70 (m, 1H), 1.69-1.59 (m, 1H), 1.00 (d, J=2.4 Hz, 6H).

### Example 232: 1-(8-((5-((4-(dimethylamino)piperidin-1-yl)sulfonyl)-4H-1,2,4-triazol-3-yl)amino)-1,2,3,5,6,7-hexahydro-s-indacen-4-yl)ethan-1-one

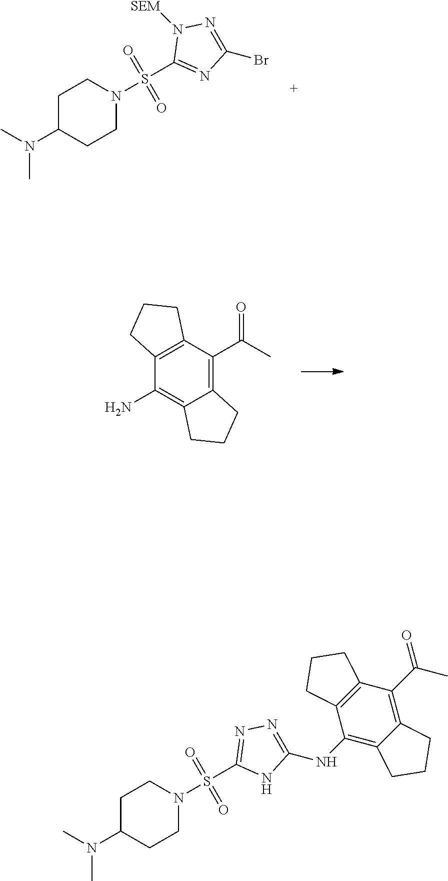 [SMILES: CN(C)C1CCN(S(=O)(=O)c2nc(Br)nn2COCC[Si](C)(C)C)CC1 ; CC(=O)c1c2c(c(N)c3c1CCC3)CCC2 ; CC(=O)c1c2c(c(Nc3nnc(S(=O)(=O)N4CCC(N(C)C)CC4)[nH]3)c3c1CCC3)CCC2]

A solution of 1-((3-bromo-1-((2-(trimethylsilyl)ethoxy)methyl)-1H-1,2,4-triazol-5-yl)sulfonyl)-N,N-dimethylpiperidin-4-amine (Intermediate A11) (653 mg, 1.39 mmol) in 1,4-dioxane (12 mL) was added to a mixture of 1-(8-amino-1,2,3,5,6,7-hexahydro-s-indacen-4-yl)ethanone (Intermediate C14) (300 mg, 1.39 mmol), Pd-175 (218 mg, 0.279 mmol) and K₂CO₃ (578 mg, 4.18 mmol) under N₂, the solution was evacuated and backfilled with N₂ three times and stirred at 85° C. for 16 h. The organic phase was washed with saturated NH₄Cl solution (10 mL), extracted with EtOAc (3×10 mL), dried (MgSO₄) and concentrated in vacuo. The residue was purified by chromatography on silica gel (24 g column, 0-10% (0.7 M ammonia/MeOH)/DCM) and the product was taken up in TFA (2 mL) and stirred at RT for 30 min. The volatiles were evaporated and the crude product was purified by acidic prep HPLC (10-40% MeOH in water) to afford the title compound (105 mg, 16%) as a white solid.

LCMS m/z 473.3 (M+H)⁺ (ES⁺).

¹H NMR (DMSO-d₆) δ 9.24 (s, 1H), 4.10 (br s, 1H), 3.80 (br d, J=12.3 Hz, 2H), 3.00 (t, J=7.4 Hz, 4H), 2.76 (td, J=12.6, 2.4 Hz, 2H), 2.69-2.61 (m, 10H), 2.48 (s, 3H), 2.03-1.96 (m, 6H), 1.70-1.58 (m, 2H). One exchangeable proton not observed.

### Example 2, 1: (S)-2-(1-((5-((1,2,3,5,6,7-hexahydro-s-indacen-4-yl)amino)-4H-1,2,4-triazol-3-yl)sulfonyl)pyrrolidin-3-yl)propan-2-ol

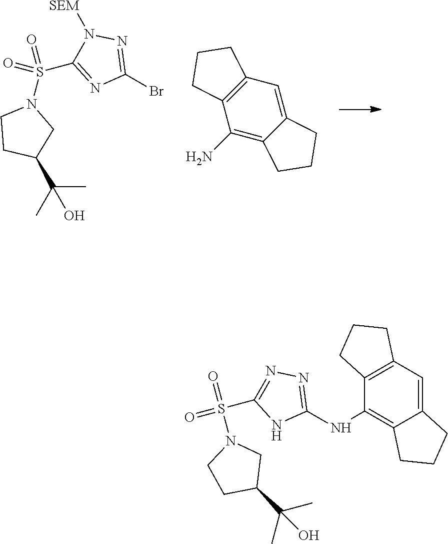

Prepared according to the general procedure of N-(5-(2-(methoxy-d)pyridin-4-yl)-2,3-dihydrobenzofuran-4-yl)-5-((6-methyl-2,6-diazaspiro[3.4]octan-2-yl)sulfonyl)-4H-1,2,4-triazol-3-amine (Example 195) from (S)-2-(1-((3-bromo-1-((2-(trimethylsilyl)-ethoxy)methyl)-1H-1,2,4-triazol-5-yl)sulfonyl)pyrrolidin-3-yl)propan-2-ol (Intermediate A11) and 1,2,3,5,6,7-hexahydro-s-indacen-4-amine to afford the title compound (10 mg, 10%) as a white solid.

LCMS m/z 432.3 (M+H)⁺ (ES⁺); 430.2 (M−H)⁻ (ES⁻).

¹H NMR (DMSO-d₆) δ 13.06 (s, 1H), 8.90 (s, 1H), 6.95 (s, 1H), 4.35 (s, 1H), 3.48-3.40 (m, 1H), 3.39-3.33 (m, 1H), 3.28-3.22 (m, 1H), 3.10 (t, J=10.0 Hz, 1H), 2.82 (t, J=7.4 Hz, 4H), 2.63 (t, J=7.3 Hz, 4H), 2.10-2.00 (m, 1H), 1.97 (p, J=7.4 Hz, 4H), 1.81-1.73 (m, 1H), 1.71-1.62 (m, 1H), 1.01 (s, 6H).

### Example 234: N-(5-(2-(benzyloxy)pyridin-4-yl)-2,3-dihydro-1H-inden-4-yl)-5-(methylsulfonyl)-4H-1,2,4-triazol-3-amine

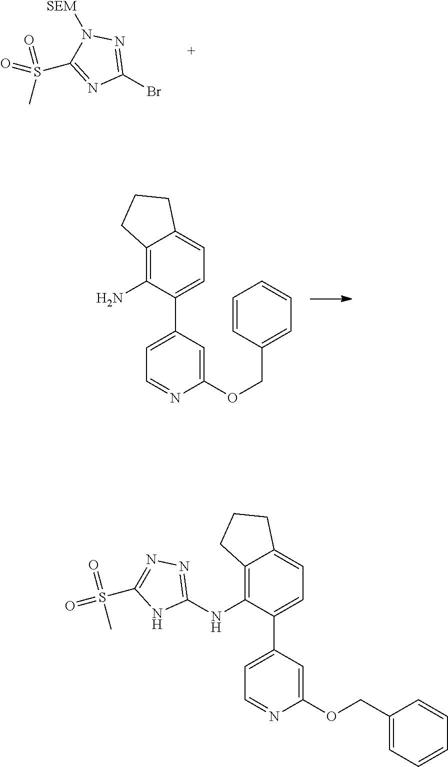 [SMILES: C[Si](C)(C)CCOCn1nc(Br)nc1S(C)(=O)=O ; Nc1c(-c2ccnc(OCc3ccccc3)c2)ccc2c1CCC2 ; CS(=O)(=O)c1nnc(Nc2c(-c3ccnc(OCc4ccccc4)c3)ccc3c2CCC3)[nH]1]

Prepared according to the general procedure of N-(5-(2-(methoxy-d)pyridin-4-yl)-2,3-dihydrobenzofuran-4-yl)-5-((6-methyl-2,6-diazaspiro[3.4]octan-2-yl)sulfonyl)-4H-1,2,4-triazol-3-amine (Example 195) from 3-bromo-5-(methylsulfonyl)-1-((2-(trimethylsilyl)ethoxy)methyl)-1H-1,2,4-triazole (Intermediate A16) and 5-(2-(benzyloxy)pyridin-4-yl)-2,3-dihydro-1H-inden-4-amine (Intermediate C28) to afford the title compound (46 mg, 35%) as a white solid.

LCMS m/z 462.3 (M+H)⁺ (ES⁺); 460.2 (M−H)⁻ (ES⁻).

¹H NMR (DMSO-d₆) δ 13.10 (br s, 1H), 9.03 (s, 1H), 8.11 (d, J=5.3 Hz, 1H), 7.47-7.35 (m, 4H), 7.33-7.30 (m, 1H), 7.27 (d, J=7.7 Hz, 1H), 7.21 (d, J=7.7 Hz, 1H), 6.93 (dd, J=5.3, 1.6 Hz, 1H), 6.83-6.79 (m, 1H), 5.34 (s, 2H), 3.13 (s, 3H), 2.96 (t, J=7.4 Hz, 2H), 2.71 (t, J=7.4 Hz, 2H), 2.04 (p, J=7.4 Hz, 2H).

### Example 235: N-(5-(2-((1-methylpiperidin-4-yl)oxy)pyridin-4-yl)-2,3-dihydro-1H-inden-4-yl)-5-(methylsulfonyl)-4H-1,2,4-triazol-3-amine

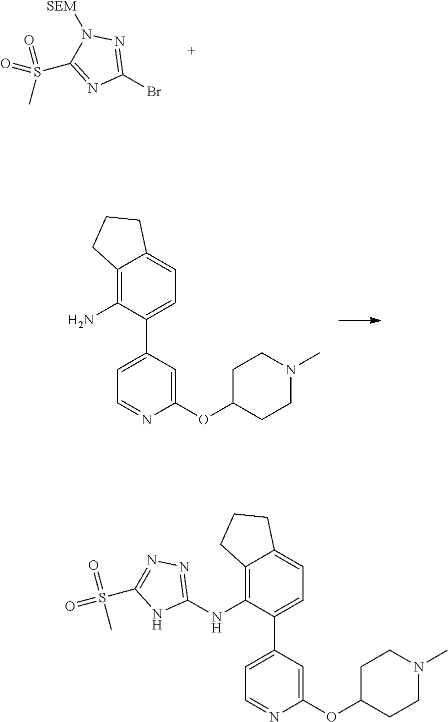 [SMILES: C[Si](C)(C)CCOCn1nc(Br)nc1S(C)(=O)=O ; CN1CCC(Oc2cc(-c3ccc4c(c3N)CCC4)ccn2)CC1 ; CN1CCC(Oc2cc(-c3ccc4c(c3Nc3nnc(S(C)(=O)=O)[nH]3)CCC4)ccn2)CC1]

Prepared according to the general procedure of N-(5-(2-(methoxy-d)pyridin-4-yl)-2,3-dihydrobenzofuran-4-yl)-5-((6-methyl-2,6-diazaspiro[3.4]octan-2-yl)sulfonyl)-4H-1,2,4-triazol-3-amine (Example 195) from 3-bromo-5-(methylsulfonyl)-1-((2-(trimethylsilyl)ethoxy)methyl)-1H-1,2,4-triazole (Intermediate A16) and 5-(2-((1-methylpiperidin-4-yl)oxy)pyridin-4-yl)-2,3-dihydro-1H-inden-4-amine (Intermediate C29) to afford the title compound (37 mg, 26%) as a white solid.

LCMS m/z 469.3 (M+H)⁺ (ES⁺); 467.3 (M−H)⁻ (ES⁻).

¹H NMR (DMSO-d₆) δ 9.05 (s, 1H), 8.15 (d, J=5.3 Hz, 1H), 7.27 (d, J=7.7 Hz, 1H), 7.20 (d, J=7.6 Hz, 1H), 6.92 (dd, J=5.3, 1.5 Hz, 1H), 6.76-6.71 (m, 1H), 5.17-5.09 (m, 1H), 3.20-3.10 (m, 5H), 3.05-2.90 (m, 4H), 2.73 (t, J=7.4 Hz, 2H), 2.67 (s, 3H), 2.14-2.00 (m, 4H), 1.92-1.80 (m, 2H). One exchangeable proton not observed.

### Example 236: 5-(methylsulfonyl)-N-(5-(2-((tetrahydro-2H-pyran-3-yl)oxy)pyridin-4-yl)-2,3-dihydro-1H-inden-4-yl)-4H-1,2,4-triazol-3-amine

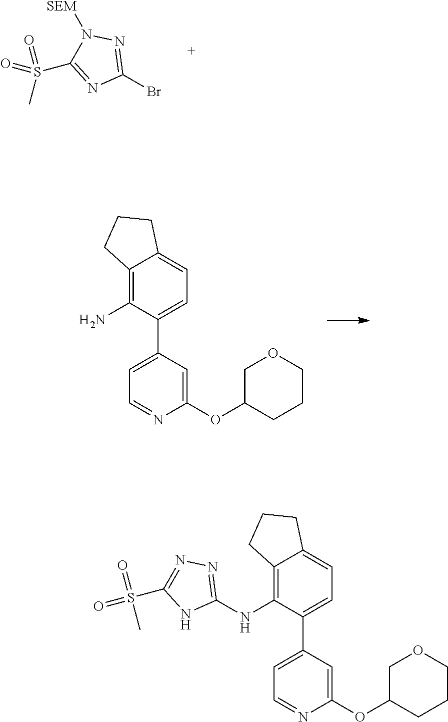 [SMILES: C[Si](C)(C)CCOCn1nc(Br)nc1S(C)(=O)=O ; Nc1c(-c2ccnc(OC3CCCOC3)c2)ccc2c1CCC2 ; CS(=O)(=O)c1nnc(Nc2c(-c3ccnc(OC4CCCOC4)c3)ccc3c2CCC3)[nH]1]

Prepared according to the general procedure of N-(5-(2-(methoxy-d)pyridin-4-yl)-2,3-dihydrobenzofuran-4-yl)-5-((6-methyl-2,6-diazaspiro[3-4]octan-2-yl)sulfonyl)-4H-1,2,4-triazol-3-amine (Example 195) from 3-bromo-5-(methylsulfonyl)-1-((2-(trimethylsilyl)ethoxy)methyl)-1H-1,2,4-triazole (Intermediate A16) and 5-(2-((tetrahydro-2H-pyran-3-yl)oxy)pyridin-4-yl)-2,3-dihydro-1H-inden-4-amine (Intermediate C30) to afford the title compound (49 mg, 31%) as a white solid.

LCMS m/z 456.2 (M+H)⁺ (ES⁺); 454.2 (M−H)⁻ (ES⁻).

¹H NMR (DMSO-d₆) δ 13.09 (br s, 1H), 9.01 (s, 1H), 8.07 (dd, J=5.3, 0.7 Hz, 1H), 7.26 (d, J=7.7 Hz, 1H), 7.20 (d, J=7.7 Hz, 1H), 6.88 (dd, J=5.3, 1.5 Hz, 1H), 6.70-6.67 (m, 1H), 5.00-4.94 (m, 1H), 3.87-3.82 (m, 1H), 3.68-3.60 (m, 1H), 3.54-3.44 (m, 2H), 3.15 (s, 3H), 2.96 (t, J=7.4 Hz, 2H), 2.70 (t, J=7.4 Hz, 2H), 2.09-1.95 (m, 3H), 1.81-1.86 (m, 2H), 1.58-1.49 (m, 1H).

### Example 237: N-(5-(2-((1-methoxypropan-2-yl)oxy)pyridin-4-yl)-2,3-dihydro-1H-inden-4-yl)-5-(methylsulfonyl)-4H-1,2,4-triazol-3-amine

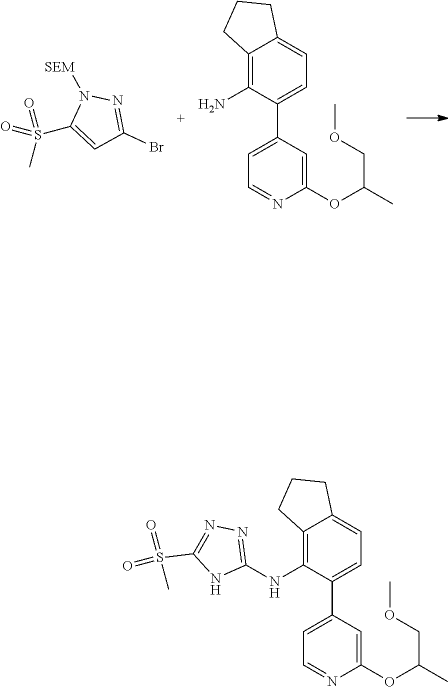 [SMILES: C[Si](C)(C)CCOCn1nc(Br)cc1S(C)(=O)=O ; COCC(C)Oc1cc(-c2ccc3c(c2N)CCC3)ccn1 ; COCC(C)Oc1cc(-c2ccc3c(c2Nc2nnc(S(C)(=O)=O)[nH]2)CCC3)ccn1]

Prepared according to the general procedure of N-(5-(2-(methoxy-d)pyridin-4-yl)-2,3-dihydrobenzofuran-4-yl)-5-((6-methyl-2,6-diazaspiro[3.4]octan-2-yl)sulfonyl)-4H-1,2,4-triazol-3-amine (Example 195) from 3-bromo-5-(methylsulfonyl)-1-((2-(trimethylsilyl)ethoxy)methyl)-1H-1,2,4-triazole (Intermediate A16) and 5-(2-((1-methoxypropan-2-yl)oxy)pyridin-4-yl)-2,3-dihydro-1H-inden-4-amine (Intermediate C31) to afford the title compound (51 mg, 34%) as a white solid.

LCMS m/z 444.3 (M+H)⁺ (ES⁺); 442.2 (M−H)⁻ (ES⁻).

¹H NMR (DMSO-d₆) δ 13.08 (br s, 1H), 9.00 (s, 1H), 8.08 (dd, J=5.3, 0.7 Hz, 1H), 7.26 (d, J=7.7 Hz, 1H), 7.20 (d, J=7.7 Hz, 1H), 6.89 (dd, J=5.3, 1.5 Hz, 1H), 6.70-6.67 (m, 1H), 5.33-5.25 (m, 1H), 3.55-3.48 (m, 1H), 3.46-3.40 (m, 1H), 3.27 (s, 3H), 3.15 (s, 3H), 2.96 (t, J=7.4 Hz, 2H), 2.70 (t, J=7.4 Hz, 2H), 2.04 (p, J=7.4 Hz, 2H), 1.23 (d, J=6.4 Hz, 3H).

### Example 238: 2-((4-(4-((5-(methylsulfonyl)-4H-1,2,4-triazol-3-yl)amino)-2,3-dihydro-1H-inden-5-yl)pyridin-2-yl)oxy)ethan-1-ol

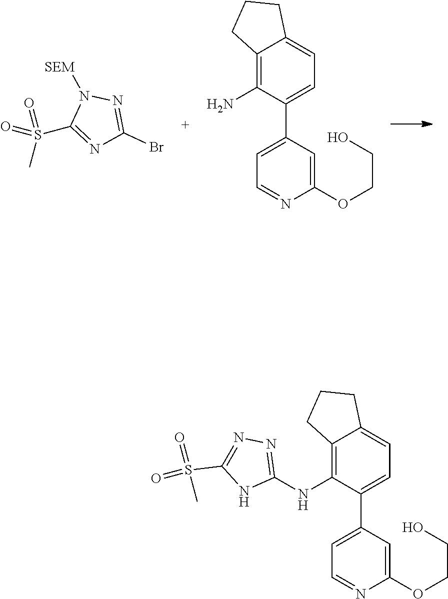 [SMILES: C[Si](C)(C)CCOCn1nc(Br)nc1S(C)(=O)=O ; Nc1c(-c2ccnc(OCCO)c2)ccc2c1CCC2 ; CS(=O)(=O)c1nnc(Nc2c(-c3ccnc(OCCO)c3)ccc3c2CCC3)[nH]1]

Prepared according to the general procedure of N-(5-(2-(methoxy-d)pyridin-4-yl)-2,3-dihydrobenzofuran-4-yl)-5-((6-methyl-2,6-diazaspiro[3-4]octan-2-yl)sulfonyl)-4H-1,2,4-triazol-3-amine (Example 195) from 3-bromo-5-(methylsulfonyl)-1-((2-(trimethylsilyl)ethoxy)methyl)-1H-1,2,4-triazole (Intermediate A16) and 2-((4-(4-amino-2,3-dihydro-1H-inden-5-yl)pyridin-2-yl)oxy)ethanol (Intermediate C32) to afford the title compound (18 mg, 22%) as a white solid.

LCMS m/z 416.1 (M+H)⁺ (ES⁺); 414.1 (M−H)⁻ (ES⁻).

¹H NMR (DMSO-d₆) δ 9.03 (s, 1H), 8.08 (dd, J=5.3, 0.7 Hz, 1H), 7.27 (d, J=7.7 Hz, 1H), 7.20 (d, J=7.7 Hz, 1H), 6.90 (dd, J=5.3, 1.5 Hz, 1H), 6.74-6.72 (m, 1H), 4.81 (br s, 1H), 4.25 (t, J=5.2 Hz, 2H), 3.70 (t, J=5.2 Hz, 2H), 3.15 (s, 3H), 2.96 (t, J=7.4 Hz, 2H), 2.70 (t, J=7.4 Hz, 2H), 2.04 (p, J=7.4 Hz, 2H). One exchangeable proton not observed.

### Example 239: 5-((6-(2-fluoroethyl)-2,6-diazaspiro[3.4]octan-2-yl)sulfonyl)-N-(1,2,3,5,6,7-hexahydro-s-indacen-4-yl)-4H-1,2,4-triazol-3-amine

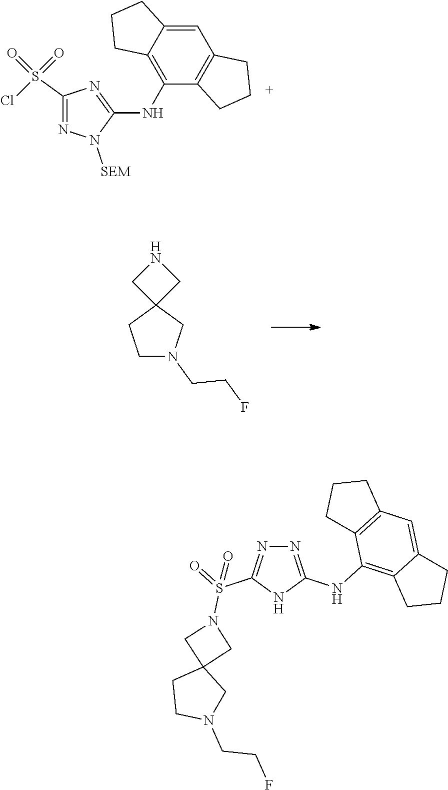 [SMILES: C[Si](C)(C)CCOCn1nc(S(=O)(=O)Cl)nc1Nc1c2c(cc3c1CCC3)CCC2 ; FCCN1CCC2(CNC2)C1 ; O=S(=O)(c1nnc(Nc2c3c(cc4c2CCC4)CCC3)[nH]1)N1CC2(CCN(CCF)C2)C1]

To a stirred solution of 6-(2-fluoroethyl)-2,6-diazaspiro[3.4]octane (Intermediate X11) (0.107 g, 0.640 mmol) and Et₃N (0.089 mL, 0.640 mmol) in DCM (2 mL) was added 5-((1,2,3,5,6,7-hexahydro-s-indacen-4-yl)amino)-1-((2-(trimethylsilyl)ethoxy)-methyl)-1H-1,2,4-triazole-3-sulfonyl chloride (Intermediate B4) (0.205 g, 0.438 mmol) in DCM (4 mL). The reaction was stirred at RT for 90 min then concentrated in vacuo. The residue was redissolved in 4 M HCl in dioxane (3 mL) and stirred for 16 h. The reaction mixture was concentrated in vacuo and the crude product was purified by acidic prep HPLC (20-50% MeOH in water) to afford the title compound (23 mg, 11%) as a white solid.

LCMS m/z 461.1 (M+H)⁺ (ES⁺).

¹H NMR (DMSO-d₆) δ 13.25 (s, 1H), 9.01 (s, 1H), 6.98 (s, 1H), 4.50 (dt, J=47.4, 4.8 Hz, 2H), 3.95-3.82 (m, 4H), 2.87-2.77 (m, 5H), 2.76-2.72 (m, 1H), 2.71-2.59 (m, 8H), 1.98 (p, J=7.4 Hz, 4H), 1.88 (t, J=7.1 Hz, 2H).

### Example 240: 5-((6-ethyl-2,6-diazaspiro[3.4]octan-2-yl)sulfonyl)-N-(1,2,3,5,6,7-hexahydro-s-indacen-4-yl)-4H-1,2,4-triazol-3-amine

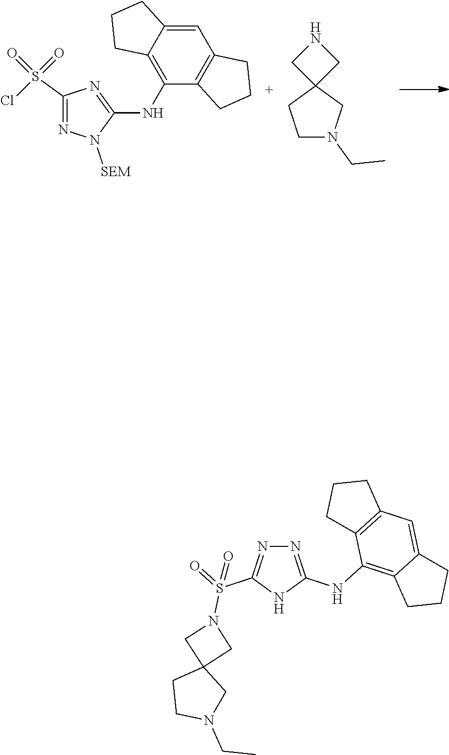

Prepared according to the general procedure of 5-((6-(2-fluoroethyl)-2,6-diazaspiro[3.4]octan-2-yl)sulfonyl)-N-(1,2,3,5,6,7-hexahydro-s-indacen-4-yl)-4H-1,2,4-triazol-3-amine (Example 239) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and 6-ethyl-2,6-diazaspiro[3.4]octane (Intermediate X15) to afford the title compound (25 mg, 13%) as a flocculent white solid.

LCMS m/z 443.3 (M+H)⁺ (ES⁺).

¹H NMR (DMSO-d₆) δ 9.02 (s, 1H), 6.98 (s, 1H), 3.96-3.86 (m, 4H), 2.95-2.74 (m, 8H), 2.75-2.60 (m, 6H), 2.06-1.88 (m, 6H), 1.05 (t, J=7.1 Hz, 3H). One exchangeable proton not observed.

### Example 241:3-((3-((dimethylamino)methyl)pyrrolidin-1-yl)sulfonyl)-N-(4-fluoro-2,6-diisopropylphenyl)-1H-1,2,4-triazol-5-amine

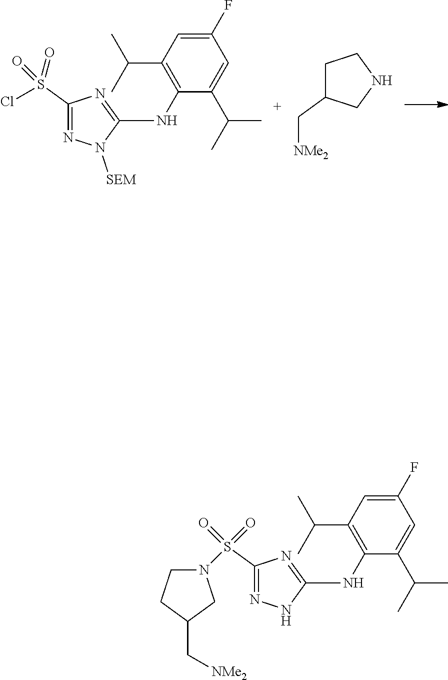

A solution of 5-((4-fluoro-2,6-diisopropylphenyl)amino)-1-((2-(trimethylsilyl)ethoxy)-methyl)-1H-1,2,4-triazole-3-sulfonyl chloride (Intermediate B55) in DCM (2 mL) was prepared at ° C. To this was added Et₃N (0.088 mL, 0.632 mmol) and N,N-dimethyl-1-(pyrrolidin-3-yl)methanamine (45 mg, 0.351 mmol) and the reaction stirred for 18 h, while warming to RT. The reaction was concentrated in vacuo, the residue was dissolved in HCl (4 M in dioxane, 2.5 mL, 82 mmol) and the resultant solution was stirred at RT for 16 h. The crude product was purified by acidic prep HPLC (20-50% MeOH in water) to afford a light yellow solid. This was loaded onto a column of SCX (0.5 g) in MeOH. The column was washed with MeOH (50 mL) and the product was eluted with 7 M ammonia in MeOH (50 mL). The ammoniacal solution was concentrated in vacuo to afford the title compound (49 mg, 34%) as a light yellow solid.

LCMS m/z 453.4 (M+H)⁺ (ES⁺); 451.3 (M−H)⁻ (ES⁻).

¹H NMR (DMSO-d₆) δ 12.85 (br s, 1H), 8.75 (s, 1H), 7.03 (d, J=9.9 Hz, 2H), 3.45-3.33 (m, 3H), 3.29-3.21 (m, 1H), 3.06 (sept, J=6.9 Hz, 2H), 2.98-2.90 (m, 1H), 2.27-2.16 (m, 1H), 2.08 (s, 6H), 2.06-1.99 (m, 1H), 1.87-1.79 (m, 1H), 1.45-1.35 (m, 1H), 1.10 (d, J=6.9 Hz, 12H).

### Example 242: N-(4-fluoro-2,6-diisopropylphenyl)-5-((6-methyl-2,6-diazaspiro[3.4]octan-2-yl)sulfonyl)-4H-1,2,4-triazol-3-amine

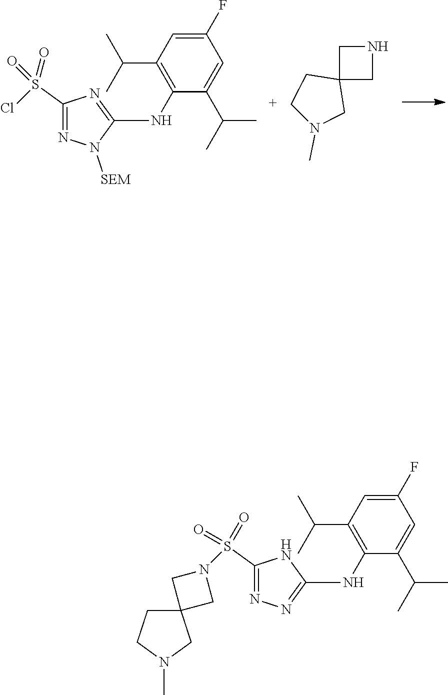

Prepared according to the general procedure of 3-((3-((dimethylamino)methyl)-pyrrolidin-1-yl)sulfonyl)-N-(4-fluoro-2,6-diisopropylphenyl)-1H-1,2,4-triazol-5-amine (Example 241) from 5-((4-fluoro-2,6-diisopropylphenyl)amino)-1-((2-(trimethylsilyl)ethoxy)methyl)-1H-1,2,4-triazole-3-sulfonyl chloride (Intermediate B⁵⁵) and 6-methyl-2,6-diazaspiro[3.4]octane (Intermediate X17) to afford the title compound (34 mg, 24%) a light yellow solid.

LCMS m/z 451.3 (M+H)⁺ (ES⁺); 449.3 (M−H)⁻ (ES⁻).

¹H NMR (DMSO-d₆) δ 12.99 (br s, 1H), 8.88 (s, 1H), 7.05 (d, J=9.9 Hz, 2H), 3.88-3.79 (m, 4H), 3.09 (sept, J=6.9 Hz, 2H), 2.42-2.32 (m, 4H), 2.17 (s, 3H), 1.81 (t, J=7.0 Hz, 2H), 1.09 (d, J=6.9 Hz, 12H).

### Example 243: 1-((5-(2-isopropyl-6-(2-methoxypyridin-4-yl)phenoxy)-4H-1,2,4-triazol-3-yl)sulfonyl)-N,N-dimethylpiperidin-4-amine

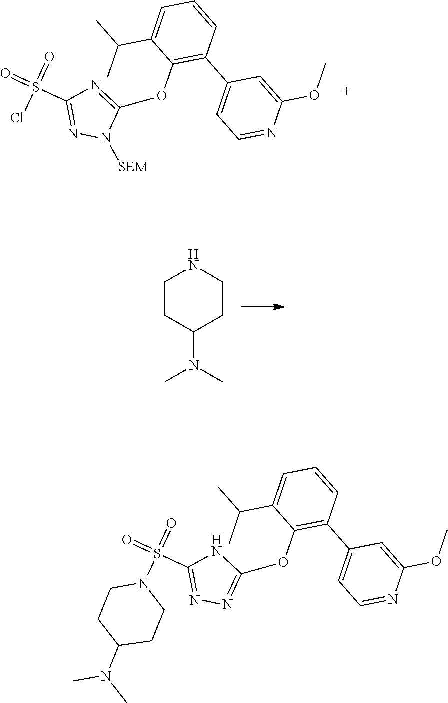

A solution of 5-(2-isopropyl-6-(2-methoxypyridin-4-yl)phenoxy)-1-((2-(trimethylsilyl)-ethoxy)methyl)-1H-1,2,4-triazole-3-sulfonyl chloride (Intermediate B52) (0.137 g, 0.254 mmol) in DCM (2 mL) was added to a solution of Et₃N (0.053 mL, 0.381 mmol) and N,N-dimethylpiperidin-4-amine (0.036 mL, 0.305 mmol) in DCM (1 mL). The reaction was stirred for 1 h then concentrated in vacuo. HCl (4M in dioxane, 2 mL, 8.00 mmol) was added to the crude material and this was stirred for 16 h. The reaction mixture was concentrated in vacuo, TFA (2 mL) was added and the solution stirred for 3 h. The reaction mixture was concentrated in vacuo and the crude product was purified by acidic prep HPLC (35-65% MeOH in water) to afford the title compound (20 mg, 15%) as a flocculent white solid.

LCMS m/z 501.3 (M+H)⁺ (ES⁺); 499.1 (M−H)⁻ (ES⁻).

¹H NMR (DMSO-d₆) δ 8.13 (d, J=5.3 Hz, 1H), 7.53 (dd, J=7.8, 1.7 Hz, 1H), 7.41 (t, J=7.7 Hz, 1H), 7.33 (dd, J=7.6, 1.7 Hz, 1H), 7.02 (dd, J=5.3, 1.5 Hz, 1H), 6.82 (br s, 1H), 3.83 (s, 3H), 3.67-3.60 (m, 2H), 3.10 (p, J=6.9 Hz, 1H), 2.97 (3.03-2.90 (m, 1H), 2.70-2.61 (m, 6H), 2.41-2.33 (m, 2H), 1.99 (m, 2H), 1.64-1.53 (m, 2H), 1.16 (d, J=6.9 Hz, 6H). One exchangeable proton not observed.

### Example 244: 5-((2-cyclopropyl-2,8-diazaspiro[4.5]decan-8-yl)sulfonyl)-N-(1,2,3,5,6,7-hexahydro-s-indacen-4-yl)-4H-1,2,4-triazol-3-amine

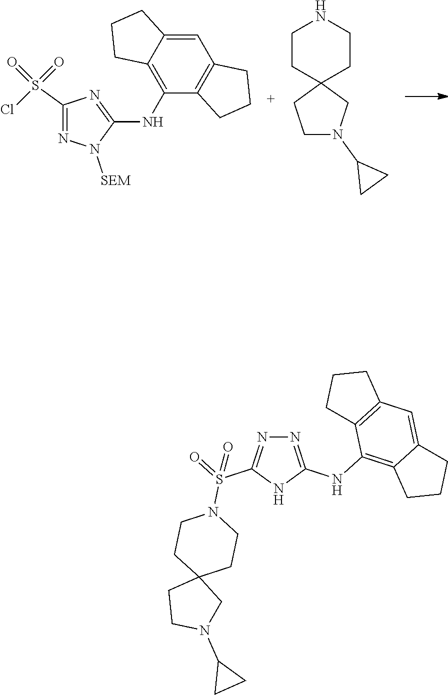

Prepared according to the general procedure of 5-((6-(2-fluoroethyl)-2,6-diazaspiro[3.4]octan-2-yl)sulfonyl)-N-(1,2,3,5,6,7-hexahydro-s-indacen-4-yl)-4H-1,2,4-triazol-3-amine (Example 239) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and 2-cyclopropyl-2,8-diazaspiro[4.5]decane (Intermediate X3) to afford the title compound (25 mg, 12%) as a white solid.

LCMS m/z 483.4 (M+H)⁺ (ES⁺).

¹H NMR (DMSO-d₆) δ 13.14 (s, 1H), 8.91 (s, 1H), 6.95 (s, 1H), 3.27-3.07 (m, 4H), 2.93 (br s, 2H), 2.82 (t, J=7.4 Hz, 4H), 2.71 (br s, 2H), 2.63 (t, J=7.3 Hz, 4H), 2.07 (app. br s, 1H), 1.97 (p, J=7.4 Hz, 4H), 1.70-1.47 (m, 6H), 0.62-0.43 (m, 4H).

### Example 241: 5-((6-cyclopropyl-2,6-diazaspiro[3.4]octan-2-yl)sulfonyl)-N-(1,2,3,5,6,7-hexahydro-s-indacen-4-yl)-4H-1,2,4-triazol-3-amine

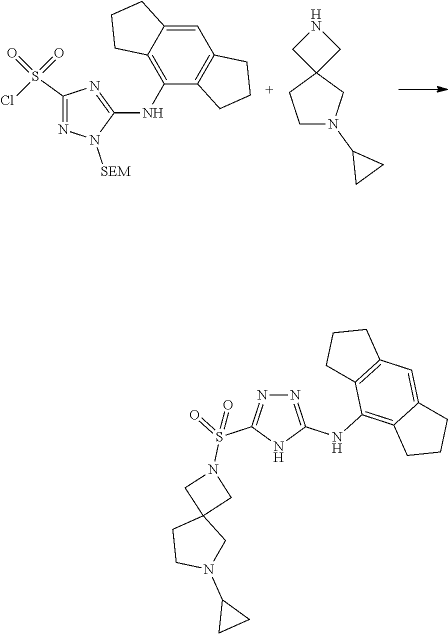

To a stirred solution of 6-cyclopropyl-2,6-diazaspiro[3.4]octane (Intermediate X12) (0.112 g, 0.640 mmol) and Et₃N (0.089 mL, 0.640 mmol) in DCM (2 mL) was added 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) (205 mg, 0.438 mmol) in DCM (4 mL). The reaction was stirred at RT for 90 min then concentrated in vacuo and the residue was purified by chromatography on silica gel (4 g column, 0-15% MeOH/DCM). The product was dissolved in 4 M HCl in dioxane (4 mL) and stirred at room temperature for 16 h. The reaction mixture was concentrated in vacuo and purified by acidic prep HPLC (20-50% MeOH in water) to afford the title compound (33 mg, 17%) as a white solid.

LCMS m/z 455.3 (M+H)⁺ (ES⁺).

¹H NMR (DMSO-d₆) δ 9.04 (s, 1H), 6.98 (s, 1H), 3.86-3.79 (m, 4H), 2.84 (t, J=7.4 Hz, 4H), 2.67 (t, J=7.3 Hz, 4H), 2.58-2.52 (m, 4H), 1.98 (p, J=7.4 Hz, 4H), 1.82 (t, J=7.1 Hz, 2H), 1.60-1.53 (m, 1H), 0.37-0.31 (m, 2H), 0.26-0.21 (m, 2H). One exchangeable proton not observed.

### Example 246: 5-((1-cyclopropyl-1,8-diazaspiro[4.5]decan-8-yl)sulfonyl)-N-(1,2,3,5,6,7-hexahydro-s-indacen-4-yl)-4H-1,2,4-triazol-3-amine

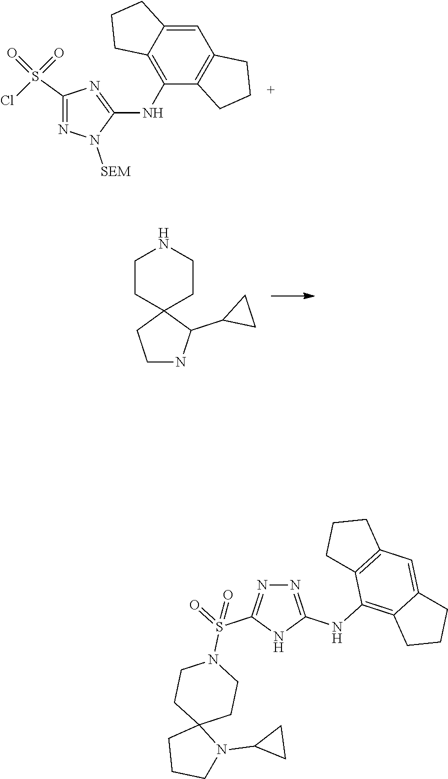 [SMILES: C[Si](C)(C)CCOCn1nc(S(=O)(=O)Cl)nc1Nc1c2c(cc3c1CCC3)CCC2 ; C1CC2(CCN1)CCNC2C1CC1 ; O=S(=O)(c1nnc(Nc2c3c(cc4c2CCC4)CCC3)[nH]1)N1CCC2(CCCN2C2CC2)CC1]

Prepared according to the general procedure of 5-((6-cyclopropyl-2,6-diazaspiro[3.4]octan-2-yl)sulfonyl)-N-(1,2,3,5,6,7-hexahydro-s-indacen-4-yl)-4H-1,2,4-triazol-3-amine (Example 245) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and 1-cyclopropyl-1,8-diazaspiro[4.5]decane (Intermediate X2) to afford the title compound (42 mg, 20%) as a white solid.

LCMS m/z 483.4 (M+H)⁺ (ES⁺).

¹H NMR (DMSO-d₆) δ 8.93 (s, 1H), 6.95 (s, 1H), 3.72-3.65 (m, 2H), 2.86-2.74 (m, 8H), 2.63 (t, J=7.3 Hz, 4H), 1.96 (p, J=7.4 Hz, 4H), 1.86 (td, J=13.0, 4.5 Hz, 2H), 1.81-1.75 (m, 1H), 1.65-1.55 (m, 4H), 1.39-1.32 (m, 2H), 0.45-0.38 (m, 2H), 0.28-0.22 (m, 2H). One exchangeable proton not observed.

### Example 247:N-(4-fluoro-2,6-diisopropylphenyl)-5-((i-methyl-1,8-diazaspiro[4.5]decan-8-yl)sulfonyl)-4H-1,2,4-triazol-3-amine

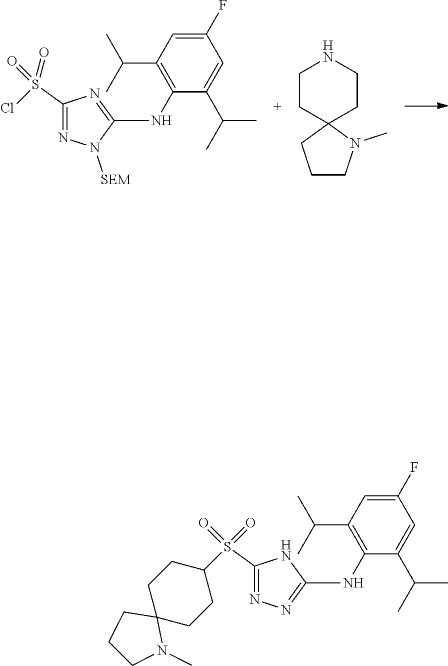

Prepared according to the general procedure of 3-((3-((dimethylamino)methyl)-pyrrolidin-1-yl)sulfonyl)-N-(4-fluoro-2,6-diisopropylphenyl)-1H-1,2,4-triazol-5-amine (Example 241) from 5-((4-fluoro-2,6-diisopropylphenyl)amino)-1-((2-(trimethylsilyl)ethoxy)methyl)-1H-1,2,4-triazole-3-sulfonyl chloride (Intermediate B⁵⁵) and 1-methyl-1,8-diazaspiro[4.5]decane (Intermediate X19) to afford the title compound (42 mg, 26%) as a light yellow solid.

LCMS m/z 479.4 (M+H)⁺ (ES⁺); 477.2 (M−H)⁻ (ES⁻).

¹H NMR (DMSO-d₆) δ 8.80 (s, 1H), 7.02 (d, J=9.9 Hz, 2H), 3.73-3.64 (m, 2H), 3.05 (sept, J=6.9 Hz, 2H), 2.82-2.68 (m, 4H), 2.23 (s, 3H), 1.75-1.52 (m, 6H), 1.34-1.23 (m, 2H), 1.09 (d, J=6.9 Hz, 12H). One exchangeable proton not observed.

### Example 248: N-(4-fluoro-2-isopropyl-6-(2-methoxypyridin-4-yl)phenyl)-5-((6-methyl-2,6-diazaspiro[3.4]octan-2-yl)sulfonyl)-4H-1,2,4-triazol-3-amine

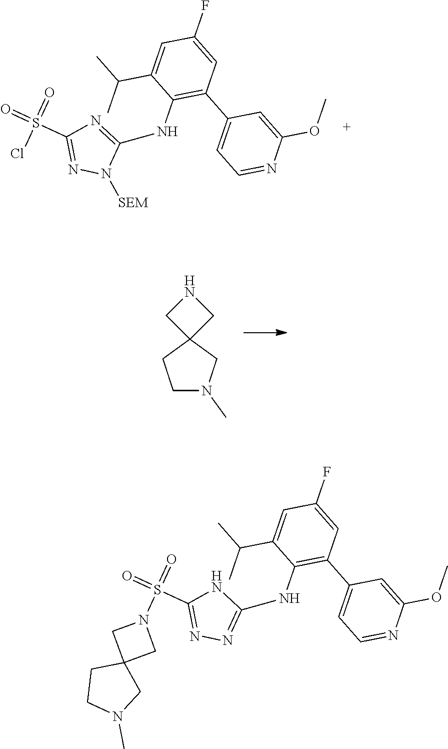

A solution of 5-((4-fluoro-2-isopropyl-6-(2-methoxypyridin-4-yl)phenyl)amino)-1-((2-(trimethylsilyl)ethoxy)methyl)-1H-1,2,4-triazole-3-sulfonyl chloride (Intermediate B⁵⁶) (103 mg, 0.186 mmol) in DCM (2 mL) was added to a solution of Et₃N (0.04 mL, 0.287 mmol) and 6-methyl-2,6-diazaspiro[3.4]octane (Intermediate X17) (28.1 mg, 0.223 mmol) in DCM (1 mL). The reaction was stirred for 1 h then concentrated in vacuo. HCl (4M in dioxane, 1.5 mL, 6.00 mmol) was added to the crude material and this was stirred for 1 h. The reaction mixture was concentrated in vacuo and the crude product was purified by acidic prep HPLC (20-50% MeOH in water) to afford the title compound (8 mg, 8%) as a white solid.

LCMS m/z 516.3 (M+H)⁺ (ES⁺); 514.1 (M−H)⁻ (ES⁻).

¹H NMR (DMSO-d₆) δ 9.04 (s, 1H), 8.12 (dd, J=5.3, 0.5 Hz, 1H), 7.35 (dd, J=10.0, 3.0 Hz, 1H), 7.15 (dd, J=8.8, 3.0 Hz, 1H), 6.97 (dd, J=5.3, 1.5 Hz, 1H), 6.79 (dd, J=1.5, 0.6 Hz, 1H), 3.83 (s, 3H), 3.76 (d, J=8.3 Hz, 2H), 3.70 (d, J=8.3 Hz, 2H), 3.20-3.10 (m, 1H), 2.42-2.34 (m, 4H), 2.19 (s, 3H), 1.71 (t, J=7.1 Hz, 2H), 1.16 (d, J=6.8 Hz, 6H). One exchangeable proton not observed.

### Example 249: N-(4-fluoro-2-isopropyl-6-(2-methoxypyridin-4-yl)phenyl)-5-((1-methyl-1,7-diazaspiro[3.5]nonan-7-yl)sulfonyl)-4H-1,2,4-triazol-3-amine

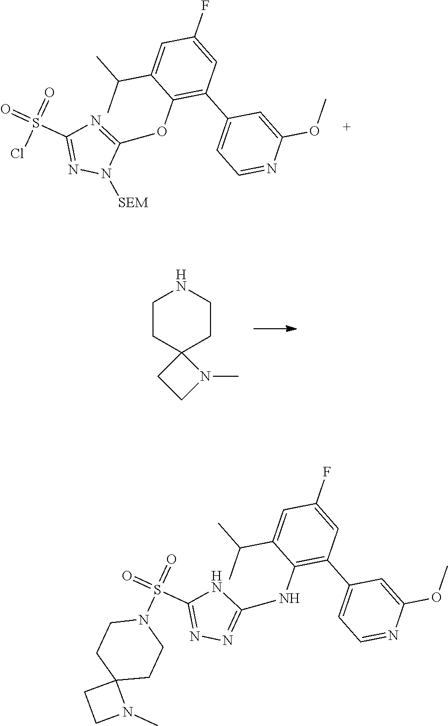

Prepared according to the general procedure of N-(4-fluoro-2-isopropyl-6-(2-methoxy-pyridin-4-yl)phenyl)-5-((6-methyl-2,6-diazaspiro[3-4]octan-2-yl)sulfonyl)-1H-1,2,4-triazol-3-amine (Example 248) from 5-((4-fluoro-2-isopropyl-6-(2-methoxypyridin-4-yl)phenyl)amino)-1-((2-(trimethylsilyl)ethoxy)methyl)-1H-1,2,4-triazole-3-sulfonyl chloride (Intermediate B56) and 1-methyl-1,7-diazaspiro[3-5]nonane (Intermediate X20) to afford the title compound (10 mg, 10%) as a white solid.

LCMS m/z 530.3 (M+H)⁺ (ES⁺); 528.1 (M−H)⁻ (ES⁻).

¹H NMR (DMSO-d₆) δ 8.97 (s, 1H), 8.09 (d, J=5.3 Hz, 1H), 7.33 (dd, J=10.0, 3.0 Hz, 1H), 7.13 (dd, J=8.8, 3.0 Hz, 1H), 6.94 (dd, J=5.3, 1.5 Hz, 1H), 6.76 (br s, 1H), 3.82 (s, 3H), 3.55-3.46 (m, 2H), 3.18-3.10 (m, 3H), 2.47-2.43 (m, 2H), 2.12 (s, 3H), 1.81 (t, J=7.0 Hz, 2H), 1.78-1.72 (m, 2H), 1.63-1.54 (m, 2H), 1.15 (d, J=6.9 Hz, 6H). One exchangeable proton not observed.

### Example 210:2-((5-(4-fluoro-2-isopropyl-6-(2-methoxypyridin-4-yl)phenoxy)-4H-1,2,4-triazol-3-yl)sulfonyl)-6-methyl-2,6-diazaspiro[3.4]octane

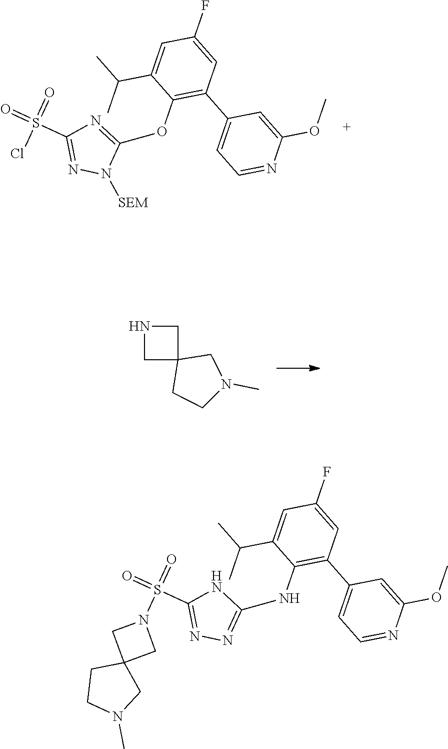

A solution of 5-(4-fluoro-2-isopropyl-6-(2-methoxypyridin-4-yl)phenoxy)-1-((2-(trimethylsilyl)ethoxy)methyl)-1H-1,2,4-triazole-3-sulfonyl chloride (Intermediate B⁷) (93 mg, 0.167 mmol) in DCM (2 mL) was added to a solution of Et₃N (0.04 mL, 0.287 mmol) and 6-methyl-2,6-diazaspiro[3.4]octane (Intermediate X17) (27.4 mg, 0.217 mmol) in DCM (1 mL). The reaction was stirred for 1 h and then concentrated in vacuo. TFA (0.013 mL, 0.167 mmol) was added and the solution was stirred for 1 h. The reaction mixture was concentrated in vacuo and the crude product was purified by acidic prep HPLC (35-65% MeOH in water) to afford the title compound (28 mg, 32%) as a white solid.

LCMS m/z 517.3 (M+H)⁺ (ES⁺); 515.1 (M−H)⁻ (ES⁻).

¹H NMR (DMSO-d₆) δ 8.12 (d, J=5.3 Hz, 1H), 7.33 (dd, J=9.4, 3.1 Hz, 1H), 7.20 (dd, J=8.7, 3.1 Hz, 1H), 7.09 (dd, J=5-4, 1.4 Hz, 1H), 6.91 (br s, 1H), 3.83 (s, 3H), 3.78 (d, J=8.4 Hz, 2H), 3.70 (d, J=8.5 Hz, 2H), 3.12-2.95 (m, 5H), 2.63 (s, 3H), 1.81 (t, J=7.1 Hz, 2H), 1.12 (d, J=6.9 Hz, 6H). One exchangeable proton not observed.

### Example 251:8-((5-(4-fluoro-2-isopropyl-6-(2-methoxypyridin-4-yl)phenoxy)-4H-1,2,4-triazol-3-yl)sulfonyl)-1-methyl-1,8-diazaspiro[4.5]decane

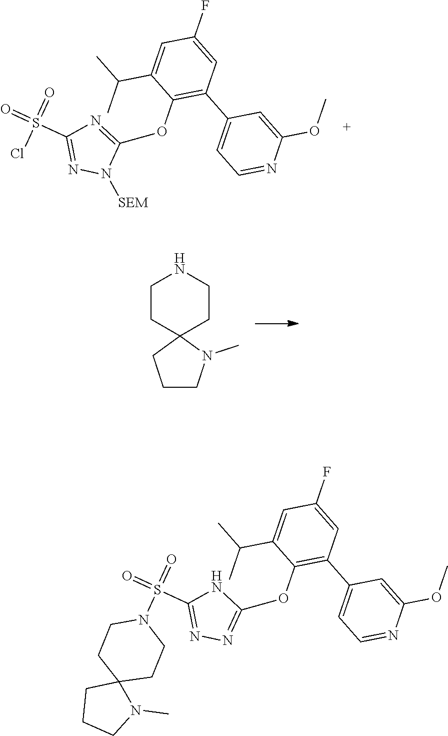 [SMILES: COc1cc(-c2cc(F)cc(C(C)C)c2Oc2nc(S(=O)(=O)Cl)nn2COCC[Si](C)(C)C)ccn1 ; CN1CCCC12CCNCC2 ; COc1cc(-c2cc(F)cc(C(C)C)c2Oc2nnc(S(=O)(=O)N3CCC4(CCCN4C)CC3)[nH]2)ccn1]

Prepared according to the general procedure of 2-((5-(4-fluoro-2-isopropyl-6-(2-methoxypyridin-4-yl)phenoxy)-4H-1,2,4-triazol-3-yl)sulfonyl)-6-methyl-2,6-diazaspiro[3.4]octane (Example 250) from 5-(4-fluoro-2-isopropyl-6-(2-methoxy-pyridin-4-yl)phenoxy)-1-((2-(trimethylsilyl)ethoxy)methyl)-1H-1,2,4-triazole-3-sulfonyl chloride (Intermediate B7) and 1-methyl-1,8-diazaspiro[4.5]decane (Intermediate X19) to afford the title compound (32 mg, 33%) as a white solid.

LCMS m/z 545.3 (M+H)⁺ (ES⁺); 543.1 (M−H)⁻ (ES⁻).

¹H NMR (DMSO-d₆) δ 8.14 (s, 1H), 8.12 (d, J=5.3 Hz, 1H), 7.33 (dd, J=9.6, 3.2 Hz, 1H), 7.20 (dd, J=8.7, 3.1 Hz, 1H), 7.06 (dd, J=5.3, 1.5 Hz, 1H), 6.87 (br s, 1H), 3.82 (s, 3H), 3.64-3.56 (m, 2H), 3.19 (br s, 1H), 3.11-3.02 (m, 1H), 2.55 (s, 3H), 2.50-2.48 (m, 2H), 1.94-1.84 (m, 2H), 1.84-1.68 (m, 4H), 1.63-1.51 (m, 2H), 1.11 (d, J=6.9 Hz, 6H). One exchangeable proton not observed.

### Example 252: 7-((5-(4-fluoro-2-isopropyl-6-(2-methoxypyridin-4-yl)phenoxy)-4H-1,2,4-triazol-3-yl)sulfonyl)-1-methyl-1,7-diazaspiro[3.5]nonane

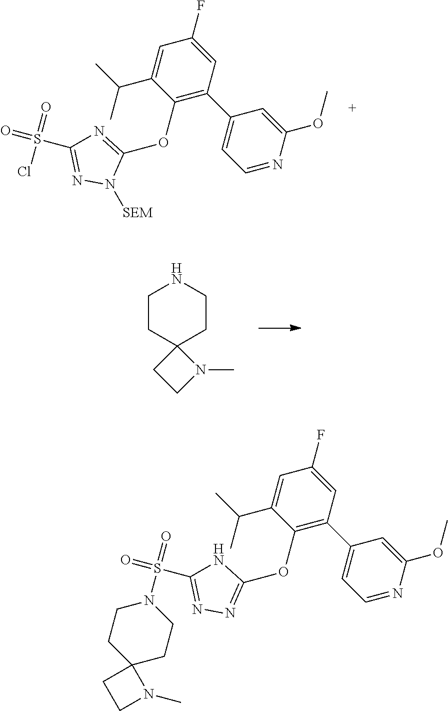

Prepared according to the general procedure of 2-((5-(4-fluoro-2-isopropyl-6-(2-methoxypyridin-4-yl)phenoxy)-4H-1,2,4-triazol-3-yl)sulfonyl)-6-methyl-2,6-diazaspiro[3.4]octane (Example 250) from 5-(4-fluoro-2-isopropyl-6-(2-methoxy-pyridin-4-yl)phenoxy)-1-((2-(trimethylsilyl)ethoxy)methyl)-1H-1,2,4-triazole-3-sulfonyl chloride (Intermediate B7) and 1-methyl-1,7-diazaspiro[3.5]nonane (Intermediate X20) to afford the title compound (31 mg, 34%) as a white solid.

LCMS m/z 531.2 (M+H)⁺ (ES⁺); 529.1 (M−H)⁻ (ES⁻).

¹H NMR (DMSO-d₆) δ 8.12 (d, J=5.3 Hz, 1H), 7.32 (dd, J=9.5, 3.1 Hz, 1H), 7.18 (dd, J=8.8, 3.1 Hz, 1H), 7.05 (d, J=5.6 Hz, 1H), 6.87 (s, 1H), 3.85 (s, 2H), 3.82 (s, 3H), 3.59-3.50 (m, 2H), 3.06 (app p, J=6.9 Hz, 1H), 2.61 (s, 3H), 2.47-2.38 (m, 2H), 2.18-2.09 (m, 4H), 1.96-1.86 (m, 2H), 1.10 (d, J=6.9 Hz, 6H). One exchangeable proton not observed.

### Example 21: 1-((5-(4-fluoro-2-isopropyl-6-(2-methoxypyridin-4-yl)phenoxy)-4H-1,2,4-triazol-3-yl)sulfonyl)-N,N-dimethylpiperidin-4-amine

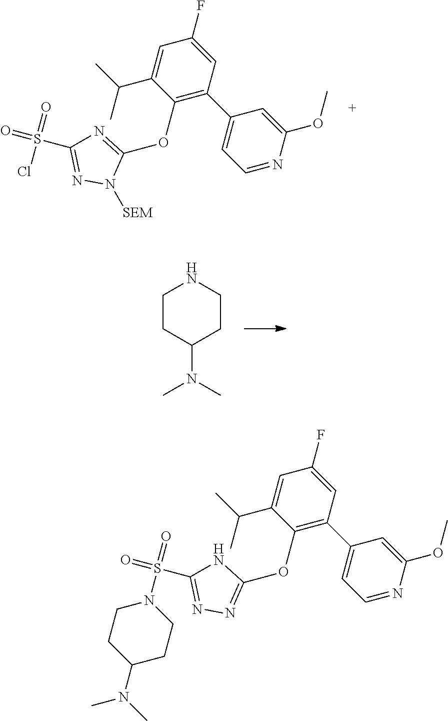

Prepared according to the general procedure of 2-((5-(4-fluoro-2-isopropyl-6-(2-methoxypyridin-4-yl)phenoxy)-4H-1,2,4-triazol-3-yl)sulfonyl)-6-methyl-2,6-diazaspiro[3.4]octane (Example 250) from 5-(4-fluoro-2-isopropyl-6-(2-methoxy-pyridin-4-yl)phenoxy)-1-((2-(trimethylsilyl)ethoxy)methyl)-1H-1,2,4-triazole-3-sulfonyl chloride (Intermediate B7) and N,N-dimethylpiperidin-4-amine to afford the title compound (25 mg, 28%) as a white solid.

LCMS m/z 519.2 (M+H)⁺ (ES⁺); δ 17.1 (M−H)⁻ (ES⁻).

¹H NMR (DMSO-d₆) δ 8.14 (d, J=5.3 Hz, 1H), 7.34 (dd, J=9.6, 3.1 Hz, 1H), 7.20 (dd, J=8.7, 3.1 Hz, 1H), 7.06 (dd, J=5.3, 1.4 Hz, 1H), 6.87 (br s, 1H), 3.83 (s, 3H), 3.66-3.55 (m, 2H), 3.13-3.05 (m, 1H), 2.89 (s, 1H), 2.63 (s, 6H), 2.41-2.31 (m, 2H), 2.01-1.91 (m, 2H), 1.61-1.49 (m, 2H), 1.13 (d, J=6.9 Hz, 6H). One exchangeable proton not observed.

### Example 214:5-((1-cyclopropyl-1,7-diazaspiro[3.5]nonan-7-yl)sulfonyl)-N-(1,2,3,5,6,7-hexahydro-s-indacen-4-yl)-4H-1,2,4-triazol-3-amine

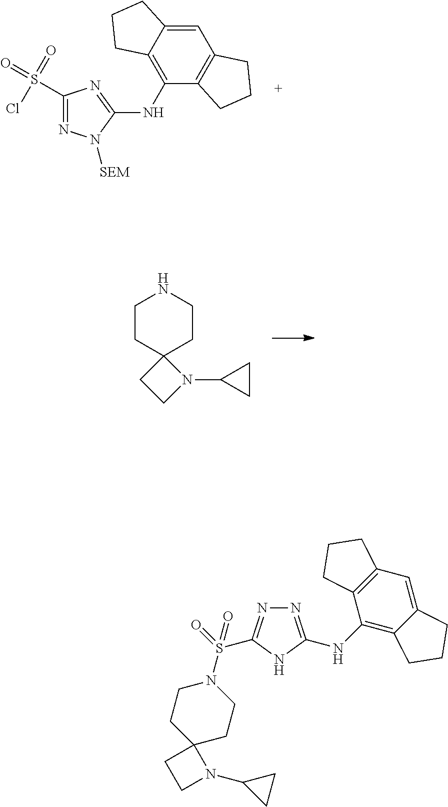 [SMILES: C[Si](C)(C)CCOCn1nc(S(=O)(=O)Cl)nc1Nc1c2c(cc3c1CCC3)CCC2 ; C1CC2(CCN1)CCN2C1CC1 ; O=S(=O)(c1nnc(Nc2c3c(cc4c2CCC4)CCC3)[nH]1)N1CCC2(CCN2C2CC2)CC1]

Prepared according to the general procedure of 5-((6-cyclopropyl-2,6-diazaspiro[3.4]octan-2-yl)sulfonyl)-N-(1,2,3,5,6,7-hexahydro-s-indacen-4-yl)-4H-1,2,4-triazol-3-amine (Example 245) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and 1-cyclopropyl-1,7-diazaspiro[3.5]nonane (Intermediate X6) to afford the title compound (22 mg, 11%) as a white solid.

LCMS m/z 469.4 (M+H)⁺ (ES⁺).

¹H NMR (DMSO-d₆) δ 8.97 (s, 1H), 6.96 (s, 1H), 3.62-3.54 (m, 2H), 3.19 (t, J=7.2 Hz, 2H), 2.87-2.74 (m, 6H), 2.63 (t, J=7.3 Hz, 4H), 1.97 (p, J=7.4 Hz, 4H), 1.93-1.88 (m, 1H), 1.84-1.71 (m, 6H), 0.33-0.27 (m, 2H), 0.19-0.12 (m, 2H). One exchangeable proton not observed.

### Example 255: 5-((1-(2-fluoroethyl)-1,8-diazaspiro[4.5]decan-8-yl)sulfonyl)-N-(1,2,3,5,6,7-hexahydro-s-indacen-4-yl)-4H-1,2,4-triazol-3-amine

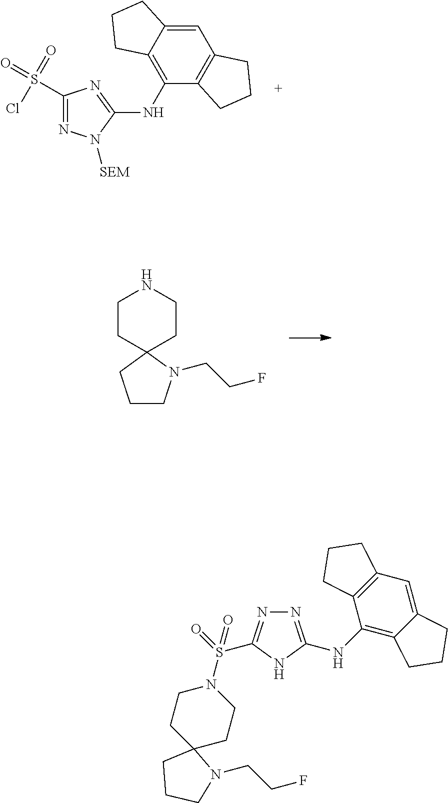 [SMILES: C[Si](C)(C)CCOCn1nc(S(=O)(=O)Cl)nc1Nc1c2c(cc3c1CCC3)CCC2 ; FCCN1CCCC12CCNCC2 ; O=S(=O)(c1nnc(Nc2c3c(cc4c2CCC4)CCC3)[nH]1)N1CCC2(CCCN2CCF)CC1]

Prepared according to the general procedure of 5-((6-(2-fluoroethyl)-2,6-diazaspiro[3.4]octan-2-yl)sulfonyl)-N-(1,2,3,5,6,7-hexahydro-s-indacen-4-yl)-4H-1,2,4-triazol-3-amine (Example 239) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and 1-(2-fluoroethyl)-1,8-diazaspiro[4.5]decane (Intermediate X4) to afford the title compound (58 mg, 26%) as a pale yellow solid.

LCMS m/z 489.4 (M+H)⁺ (ES⁺).

¹H NMR (DMSO-d₆) δ 13.09 (s, 1H), 8.93 (s, 1H), 6.97 (s, 1H), 4.47 (dt, J=47.4, 5.1 Hz, 2H), 3.77-3.64 (m, 2H), 2.87-2.74 (m, 8H), 2.75-2.70 (m, 1H), 2.69-2.61 (m, 5H), 1.98 (p, J=7.4 Hz, 4H), 1.70 (p, J=7.3 Hz, 2H), 1.62-1.53 (m, 4H), 1.35-1.26 (m, 2H).

### Example 256: 5-((1-ethyl-1,8-diazaspiro[4.5]decan-8-yl)sulfonyl)-N-(1,2,3,5,6,7-hexahydro-s-indacen-4-yl)-4H-1,2,4-triazol-3-amine

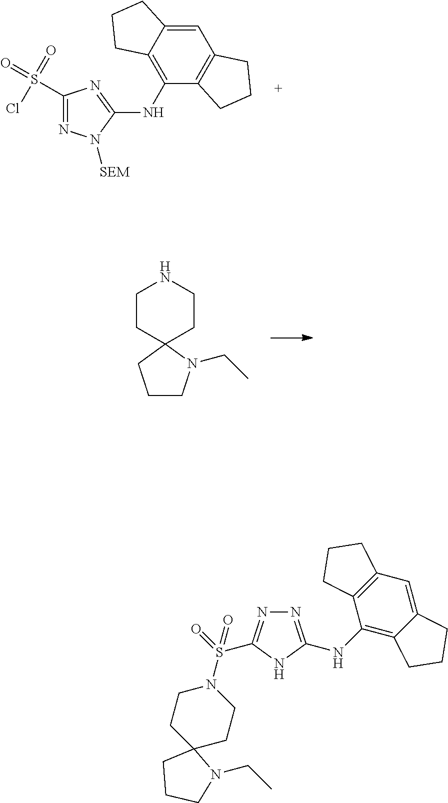 [SMILES: C[Si](C)(C)CCOCn1nc(S(=O)(=O)Cl)nc1Nc1c2c(cc3c1CCC3)CCC2 ; CCN1CCCC12CCNCC2 ; CCN1CCCC12CCN(S(=O)(=O)c1nnc(Nc3c4c(cc5c3CCC5)CCC4)[nH]1)CC2]

Prepared according to the general procedure of 5-((6-(2-fluoroethyl)-2,6-diazaspiro[3.4]octan-2-yl)sulfonyl)-N-(1,2,3,5,6,7-hexahydro-s-indacen-4-yl)-4H-1,2,4-triazol-3-amine (Example 239) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and 1-ethyl-1,8-diazaspiro[4.5]decane (Intermediate X7) to afford the title compound (47 mg, 22%) as a pale yellow solid.

LCMS m/z 471.5 (M+H)⁺ (ES⁺).

¹H NMR (DMSO-d₆) δ 9.08 (s, 1H), 6.96 (s, 1H), 3.80-3.67 (m, 2H), 2.90 (t, J=7.0 Hz, 2H), 2.86-2.78 (m, 6H), 2.65 (t, J=7.3 Hz, 4H), 2.58 (q, J=7.0 Hz, 2H), 1.98 (p, J=7.4 Hz, 4H), 1.82-1.63 (m, 6H), 1.46-1.37 (m, 2H), 1.06 (t, J=7.1 Hz, 3H). One exchangeable proton not observed.

### Example 217: 5-((1-ethyl-1,7-diazaspiro[35]nonan-7-yl)sulfonyl)-N-(1,2,3,5,6,7-hexahydro-s-indacen-4-yl)-4H-1,2,4-triazol-3-amine

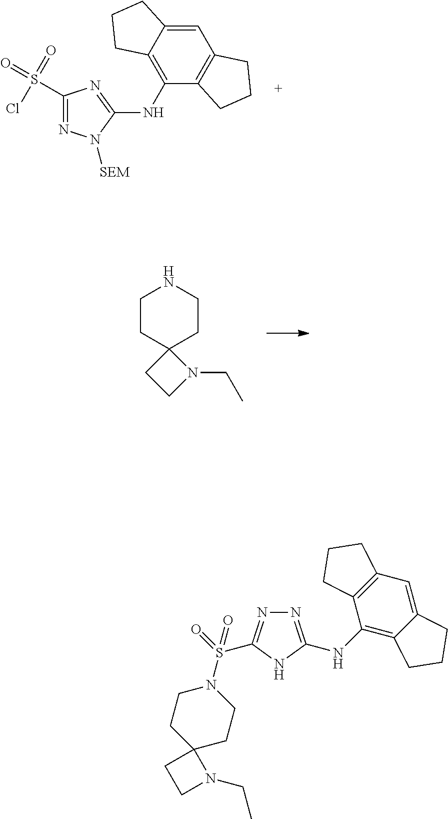 [SMILES: C[Si](C)(C)CCOCn1nc(S(=O)(=O)Cl)nc1Nc1c2c(cc3c1CCC3)CCC2 ; CCN1CCC12CCNCC2 ; CCN1CCC12CCN(S(=O)(=O)c1nnc(Nc3c4c(cc5c3CCC5)CCC4)[nH]1)CC2]

Prepared according to the general procedure of 5-((6-(2-fluoroethyl)-2,6-diazaspiro[3.4]octan-2-yl)sulfonyl)-N-(1,2,3,5,6,7-hexahydro-s-indacen-4-yl)-4H-1,2,4-triazol-3-amine (Example 239) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and 1-ethyl-1,7-diazaspiro[3.5]nonane (Intermediate X8) to afford the title compound (19 mg, 9%) as a white solid.

LCMS m/z 457.3 (M+H)⁺ (ES⁺).

¹H NMR (DMSO-d₆) δ 9.22 (s, 1H), 6.94 (s, 1H), 3.72-3.62 (m, 2H), 3.43 (t, J=7.4 Hz, 2H), 2.82 (t, J=7.4 Hz, 4H), 2.78-2.71 (m, 2H), 2.70-2.60 (m, 6H), 2.06-1.91 (m, 8H), 1.76 (td, J=12.7, 4.5 Hz, 2H), 0.92 (t, J=7.1 Hz, 3H). One exchangeable proton not observed.

### Example 218:N-(5-(2-methoxypyridin-4-yl)-2,3-dihydro-1H-inden-4-yl)-5-((1-methyl-1,8-diazaspiro[4.5]decan-8-yl)sulfonyl)-4H-1,2,4-triazol-3-amine

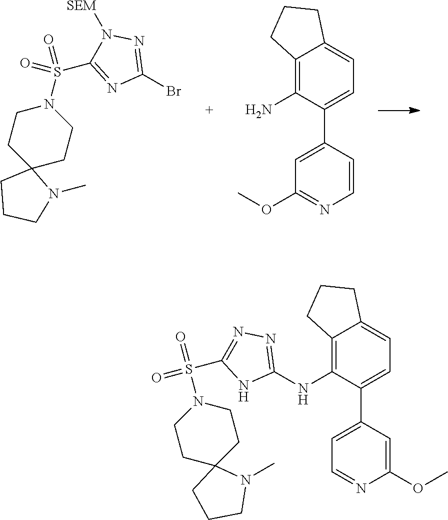 [SMILES: CN1CCCC12CCN(S(=O)(=O)c1nc(Br)nn1COCC[Si](C)(C)C)CC2 ; COc1cc(-c2ccc3c(c2N)CCC3)ccn1 ; COc1cc(-c2ccc3c(c2Nc2nnc(S(=O)(=O)N4CCC5(CCCN5C)CC4)[nH]2)CCC3)ccn1]

Prepared according to the general procedure of N-(5-(2-(methoxy-d)pyridin-4-yl)-2,3-dihydrobenzofuran-4-yl)-5-((6-methyl-2,6-diazaspiro[3.4]octan-2-yl)sulfonyl)-4H-1,2,4-triazol-3-amine (Example 195) from 8-((3-bromo-1-((2-(trimethylsilyl)ethoxy)-methyl)-1H-1,2,4-triazol-5-yl)sulfonyl)-1-methyl-1,8-diazaspiro[4.5]decane (Intermediate A15) and 5-(2-methoxypyridin-4-yl)-2,3-dihydro-1H-inden-4-amine (Intermediate R4) to afford the title compound (9 mg, 11%) as a white solid.

LCMS m/z 524.3 (M+H)⁺ (ES⁺).

¹H NMR (DMSO-d₆) δ 9.00 (s, 1H), 8.09 (d, J=5.3 Hz, 1H), 7.26 (d, J=7.7 Hz, 1H), 7.20 (d, J=7.7 Hz, 1H), 6.92 (dd, J=5.15 Hz, 1H), 6.73 (s, 1H), 3.82 (s, 3H), 3.71-3.57 (m, 2H), 2.95 (t, J=7.4 Hz, 2H), 2.77 (t, J=6.9 Hz, 2H), 2.70 (t, J=7.5 Hz, 2H), 2.66-2.58 (m, 2H), 2.21 (s, 3H), 2.02 (p, J=7.5 Hz, 2H), 1.78-1.53 (m, 6H), 1.31 (br d, J=12.5 Hz, 2H). One exchangeable proton not observed.

### Example 259: 5-((3-((dimethylamino)methyl)pyrrolidin-1-yl)sulfonyl)-N-(4-fluoro-2-isopropyl-6-(2-methoxypyridin-4-yl)phenyl)-4H-1,2,4-triazol-3-amine

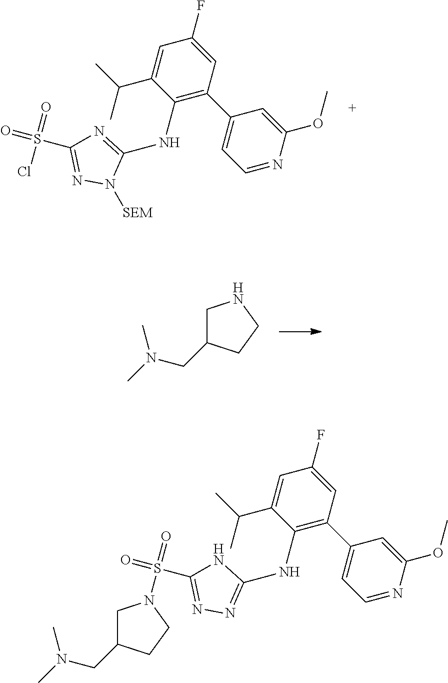

Prepared according to the general procedure of 5-((6-(2-fluoroethyl)-2,6-diazaspiro[3.4]octan-2-yl)sulfonyl)-N-(1,2,3,5,6,7-hexahydro-s-indacen-4-yl)-4H-1,2,4-triazol-3-amine (Example 239) from 5-((4-fluoro-2-isopropyl-6-(2-methoxypyridin-4-yl)phenyl)amino)-1-((2-(trimethylsilyl)ethoxy)methyl)-1H-1,2,4-triazole-3-sulfonyl chloride (Intermediate B56) and N,N-dimethyl-1-(pyrrolidin-3-yl)methanamine to afford the title compound (9 mg, 10%) as a flocculent white solid.

LCMS m/z 518.3 (M+H)⁺ (ES⁺); 516.2 (M−H)⁻ (ES⁻).

¹H NMR (DMSO-d₆) δ 8.95 (s, 1H), 8.10 (d, J=5.3 Hz, 1H), 7.33 (dd, J=9.9, 3.0 Hz, 1H), 7.14 (dd, J=8.8, 3.0 Hz, 1H), 6.95 (dd, J=5.3, 1.4 Hz, 1H), 6.77 (br s, 1H), 3.82 (s, 3H), 3.36-3.29 (m, 1H), 3.29-3.22 (m, 1H), 3.21-3.13 (m, 1H), 3.13-3.06 (m, 1H), 2.88-2.83 (m, 1H), 2.22-2.16 (m, 1H), 2.11 (s, 6H), 2.10-2.08 (m, 2H), 1.83-1.75 (m, 1H), 1.41-1.31 (m, 1H), 1.15 (d, J=6.8 Hz, 6H). One exchangeable proton not observed.

### Example 260:N-(4-fluoro-2-isopropyl-6-(2-methoxypyridin-4-yl)phenyl)-5-((1-methyl-1,8-diazaspiro[4.5]decan-8-yl)sulfonyl)-4H-1,2,4-triazol-3-amine

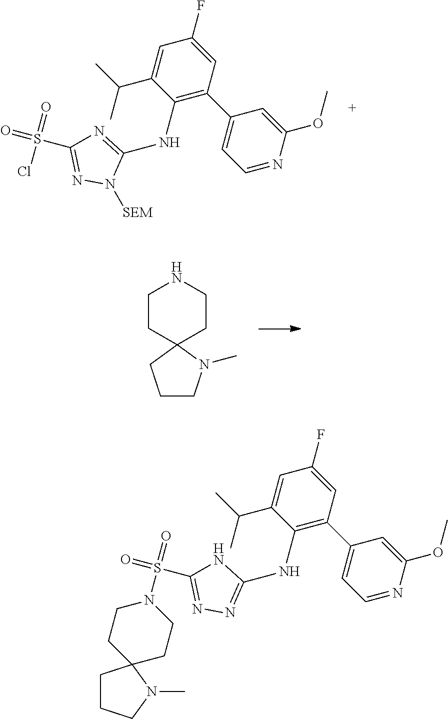 [SMILES: COc1cc(-c2cc(F)cc(C(C)C)c2Nc2nc(S(=O)(=O)Cl)nn2COCC[Si](C)(C)C)ccn1 ; CN1CCCC12CCNCC2 ; COc1cc(-c2cc(F)cc(C(C)C)c2Nc2nnc(S(=O)(=O)N3CCC4(CCCN4C)CC3)[nH]2)ccn1]

Prepared according to the general procedure of 5-((6-(2-fluoroethyl)-2,6-diazaspiro[3.4]octan-2-yl)sulfonyl)-N-(1,2,3,5,6,7-hexahydro-s-indacen-4-yl)-4H-1,2,4-triazol-3-amine (Example 239) from 5-((4-fluoro-2-isopropyl-6-(2-methoxypyridin-4-yl)phenyl)amino)-1-((2-(trimethylsilyl)ethoxy)methyl)-1H-1,2,4-triazole-3-sulfonyl chloride (Intermediate B56) and 1-methyl-1,8-diazaspiro[4.5]decane (Intermediate X19) to afford the title compound (10 mg, 9%) as a flocculent white solid.

LCMS m/z 544.3 (M+H)⁺ (ES⁺); 542.2 (M−H)⁻ (ES⁻).

¹H NMR (DMSO-d₆) δ 8.98 (s, 1H), 8.13-8.07 (m, 1H), 7.33 (dd, J=10.0, 3.0 Hz, 1H), 7.14 (dd, J=8.8, 3.0 Hz, 1H), 6.95 (dd, J=5.3, 1.4 Hz, 1H), 6.76 (s, 1H), 3.82 (s, 3H), 3.63-3.54 (m, 2H), 3.20-3.10 (m, 1H), 2.74 (t, J=7.0 Hz, 2H), 2.58-2.52 (m, 2H), 2.19 (s, 3H), 1.75-1.66 (m, 2H), 1.66-1.58 (m, 2H), 1.58-1.52 (m, 2H), 1.31-1.25 (m, 2H), 1.15 (d, J=6.8 Hz, 6H). One exchangeable proton not observed.

### Example 261: 1-((5-((4-fluoro-2-isopropyl-6-(2-methoxypyridin-4-yl)phenyl)amino)-4H-1,2,4-triazol-3-yl)sulfonyl)-N,N-dimethylpiperidin-4-amine

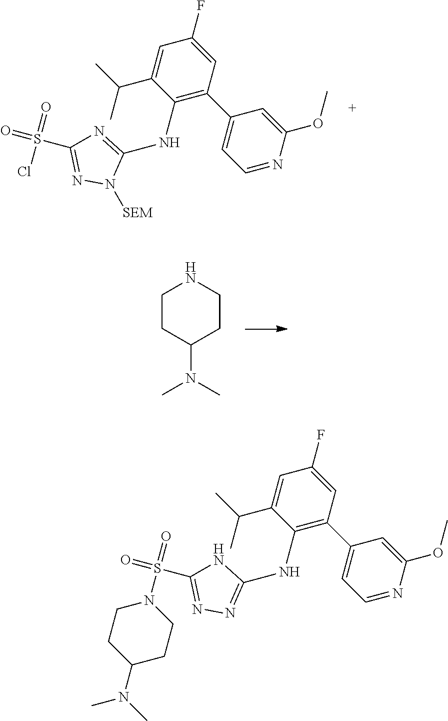

Prepared according to the general procedure of 5-((6-(2-fluoroethyl)-2,6-diazaspiro[3.4]octan-2-yl)sulfonyl)-N-(1,2,3,5,6,7-hexahydro-s-indacen-4-yl)-4H-1,2,4-triazol-3-amine (Example 239) from 5-((4-fluoro-2-isopropyl-6-(2-methoxypyridin-4-yl)phenyl)amino)-1-((2-(trimethylsilyl)ethoxy)methyl)-1H-1,2,4-triazole-3-sulfonyl chloride (Intermediate B56) and N,N-dimethylpiperidin-4-amine to afford the title compound (3 mg, 3%) as a flocculent white solid.

LCMS m/z 518.3 (M+H)⁺ (ES⁺).

¹H NMR (DMSO-d₆) δ 8.99 (s, 1H), 8.11 (dd, J=5.3, 0.7 Hz, 1H), 7.33 (dd, J=10.0, 3.0 Hz, 1H), 7.14 (dd, J=8.8, 3.0 Hz, 1H), 6.97 (dd, J=5.3, 1.4 Hz, 1H), 6.78-6.76 (m, 1H), 3.82 (s, 3H), 3.59-3.50 (m, 2H), 3.25-3.14 (m, 1H), 2.41-2.33 (m, 2H), 2.32-2.26 (m, 1H), 2.24 (s, 6H), 1.84-1.76 (m, 2H), 1.48-1.34 (m, 2H), 1.16 (d, J=6.9 Hz, 6H). One exchangeable proton not observed.

### Example 262: 5-((1,2,3,5,6,7-hexahydro-s-indacen-4-yl)amino)-N,N-dimethyl-4H-1,2,4-triazole-3-sulfonamide

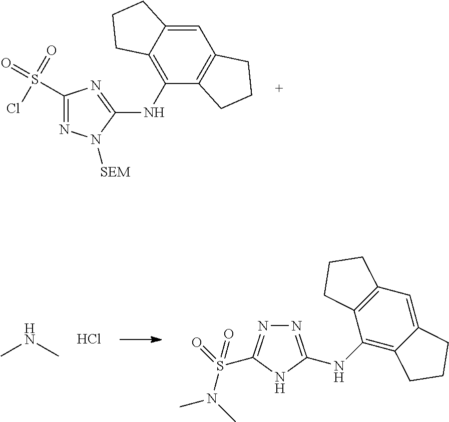

Prepared according to the general procedure of 5-((6-(2-fluoroethyl)-2,6-diazaspiro[3.4]octan-2-yl)sulfonyl)-N-(1,2,3,5,6,7-hexahydro-s-indacen-4-yl)-4H-1,2,4-triazol-3-amine (Example 239) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and dimethylamine hydrochloride to afford the title compound (24 mg, 16%) as a flocculent white solid.

LCMS m/z 348.5 (M+H)⁺ (ES⁺).

¹H NMR (DMSO-d₆) δ 13.04 (s, 1H), 8.94 (s, 1H), 6.96 (s, 1H), 2.82 (t, J=7.4 Hz, 4H), 2.78 (s, 6H), 2.64 (t, J=7.3 Hz, 4H), 1.97 (p, J=7.4 Hz, 4H).

### Example 263: N-(1,2,3,5,6,7-hexahydro-s-indacen-4-yl)-5-((6-(2,2,2-trifluoroethyl)-2,6-diazaspiro[3.4]octan-2-yl)sulfonyl)-4H-1,2,4-triazol-3-amine

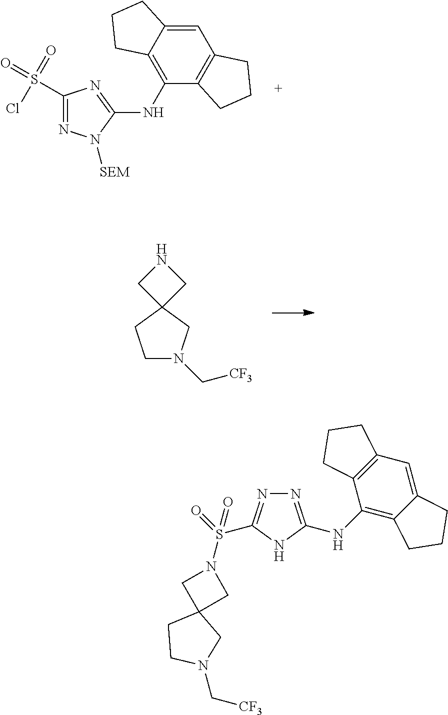 [SMILES: C[Si](C)(C)CCOCn1nc(S(=O)(=O)Cl)nc1Nc1c2c(cc3c1CCC3)CCC2 ; FC(F)(F)CN1CCC2(CNC2)C1 ; O=S(=O)(c1nnc(Nc2c3c(cc4c2CCC4)CCC3)[nH]1)N1CC2(CCN(CC(F)(F)F)C2)C1]

Prepared according to the general procedure of 5-((6-(2-fluoroethyl)-2,6-diazaspiro[3.4]octan-2-yl)sulfonyl)-N-(1,2,3,5,6,7-hexahydro-s-indacen-4-yl)-4H-1,2,4-triazol-3-amine (Example 239) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and 6-(2,2,2-trifluoroethyl)-2,6-diazaspiro[3.4]octane (Intermediate X5) to afford the title compound (37 mg, 17%) as a white solid.

LCMS m/z 497.3 (M+H)⁺ (ES⁺).

¹H NMR (DMSO-d₆) δ 13.29-13.16 (m, 1H), 9.01 (s, 1H), 6.97 (s, 1H), 3.85 (s, 4H), 3.18 (q, J=10.2 Hz, 2H), 2.83 (t, J=7.4 Hz, 4H), 2.74-2.58 (m, 8H), 1.98 (p, J=7.4 Hz, 4H), 1.86 (t, J=7.0 Hz, 2H).

### Example 264:N-(1,2,3,5,6,7-hexahydro-s-indacen-4-yl)-5-((6-isopropyl-2,6-diazaspiro[3.4]octan-2-yl)sulfonyl)-4H-1,2,4-triazol-3-amine

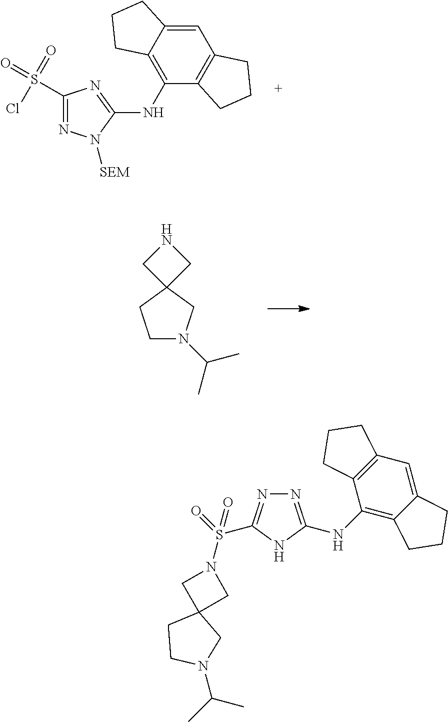 [SMILES: C[Si](C)(C)CCOCn1nc(S(=O)(=O)Cl)nc1Nc1c2c(cc3c1CCC3)CCC2 ; CC(C)N1CCC2(CNC2)C1 ; CC(C)N1CCC2(C1)CN(S(=O)(=O)c1nnc(Nc3c4c(cc5c3CCC5)CCC4)[nH]1)C2]

Prepared according to the general procedure of 5-((6-(2-fluoroethyl)-2,6-diazaspiro[3.4]octan-2-yl)sulfonyl)-N-(1,2,3,5,6,7-hexahydro-s-indacen-4-yl)-4H-1,2,4-triazol-3-amine (Example 239) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and 6-isopropyl-2,6-diazaspiro[3.4]octane (Intermediate X10) to afford the title compound (27 mg, 14%) as a flocculent white solid.

LCMS m/z 457.3 (M+H)⁺ (ES⁺).

¹H NMR (DMSO-d₆) δ 13.25 (s, 1H), 9.06 (s, 1H), 6.98 (s, 1H), 4.10 (d, J=8.8 Hz, 1H), 4.04-3.92 (m, 2H), 3.86 (d, J=8.9 Hz, 1H), 3.56-3.50 (m, 1H), 3.44-3.36 (m, 1H), 3.32-3.26 (m, 1H), 3.21-3.14 (m, 1H), 3.11-3.01 (m, 1H), 2.84 (t, J=7.4 Hz, 4H), 2.66 (t, J=7.3 Hz, 4H), 2.18-2.06 (m, 1H), 2.06-1.91 (m, 5H), 1.23 (d, J=6.5 Hz, 6H).

### Example 265:5-((2,6-diazaspiro[3.4]octan-2-yl)sulfonyl)-N-(1,2,3,5,6,7-hexahydro-s-indacen-4-yl)-4H-1,2,4-triazol-3-amine

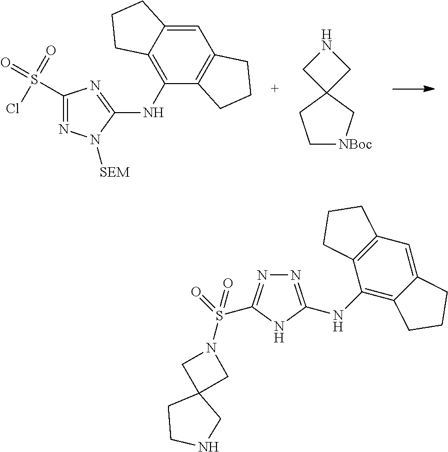 [SMILES: C[Si](C)(C)CCOCn1nc(S(=O)(=O)Cl)nc1Nc1c2c(cc3c1CCC3)CCC2 ; CC(C)(C)OC(=O)N1CCC2(CNC2)C1 ; O=S(=O)(c1nnc(Nc2c3c(cc4c2CCC4)CCC3)[nH]1)N1CC2(CCNC2)C1]

Prepared according to the general procedure of 5-((6-(2-fluoroethyl)-2,6-diazaspiro[3.4]octan-2-yl)sulfonyl)-N-(1,2,3,5,6,7-hexahydro-s-indacen-4-yl)-4H-1,2,4-triazol-3-amine (Example 239) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and tert-butyl 2,6-diazaspiro[3.4]octane-6-carboxylate to afford the title compound (45 mg, 25%) as a flocculent white solid.

LCMS m/z 415.3 (M+H)⁺ (ES⁺).

¹H NMR (DMSO-d₆) δ 9.14 (s, 1H), 6.94 (s, 1H), 3.97-3.77 (m, 4H), 3.00-2.88 (m, 4H), 2.83 (t, J=7.4 Hz, 4H), 2.66 (t, J=7.2 Hz, 4H), 1.97 (p, J=7.4 Hz, 4H), 1.84 (t, J=7.2 Hz, 2H). Two exchangeable protons not observed.

### Example 266: 5-((2,8-diazaspiro[4.5]decan-8-yl)sulfonyl)-N-(1,2,3,5,6,7-hexahydro-s-indacen-4-yl)-4H-1,2,4-triazol-3-amine

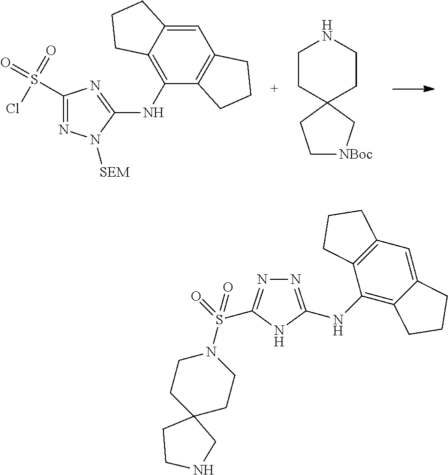 [SMILES: C[Si](C)(C)CCOCn1nc(S(=O)(=O)Cl)nc1Nc1c2c(cc3c1CCC3)CCC2 ; CC(C)(C)OC(=O)N1CCC2(CCNCC2)C1 ; O=S(=O)(c1nnc(Nc2c3c(cc4c2CCC4)CCC3)[nH]1)N1CCC2(CCNC2)CC1]

Prepared according to the general procedure of 5-((6-(2-fluoroethyl)-2,6-diazaspiro[3.4]octan-2-yl)sulfonyl)-N-(1,2,3,5,6,7-hexahydro-s-indacen-4-yl)-4H-1,2,4-triazol-3-amine (Example 239) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and tert-butyl 2,8-diazaspiro[4.5]decane-2-carboxylate to afford the title compound (51 mg, 27%) as a flocculent white solid.

LCMS m/z 443.3 (M+H)⁺ (ES⁺).

¹H NMR (DMSO-d₆) δ 9.47 (s, 1H), 6.92 (s, 1H), 3.29-3.08 (m, 6H), 2.90 (s, 2H), 2.81 (t, J=7.4 Hz, 4H), 2.66 (t, J=7.5 Hz, 4H), 1.96 (p, J=7.4 Hz, 4H), 1.70 (t, J=7.3 Hz, 2H), 1.65-1.55 (m, 4H). Two exchangeable protons not observed.

### Example 267: N-(1,2,3,5,6,7-hexahydro-s-indacen-4-yl)-5-((1-(2,2,2-trifluoroethyl)-1,8-diazaspiro[4.5]decan-8-yl)sulfonyl)-4H-1,2,4-triazol-3-amine

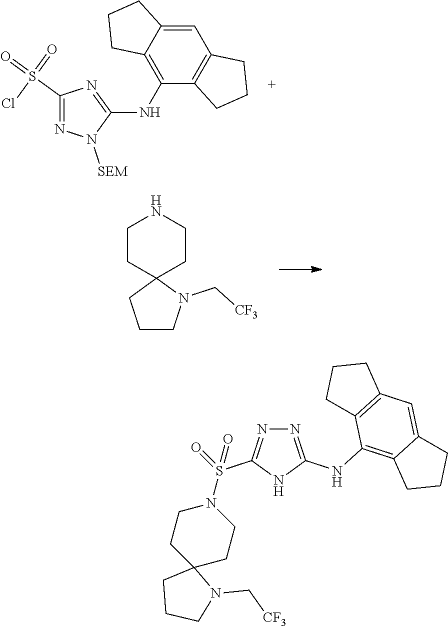 [SMILES: C[Si](C)(C)CCOCn1nc(S(=O)(=O)Cl)nc1Nc1c2c(cc3c1CCC3)CCC2 ; FC(F)(F)CN1CCCC12CCNCC2 ; O=S(=O)(c1nnc(Nc2c3c(cc4c2CCC4)CCC3)[nH]1)N1CCC2(CCCN2CC(F)(F)F)CC1]

Prepared according to the general procedure of 5-((6-(2-fluoroethyl)-2,6-diazaspiro[3.4]octan-2-yl)sulfonyl)-N-(1,2,3,5,6,7-hexahydro-s-indacen-4-yl)-4H-1,2,4-triazol-3-amine (Example 239) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and 1-(2,2,2-trifluoroethyl)-1,8-diazaspiro[4.5]decane (Intermediate X1) to afford the title compound (52 mg, 22%) as a pale yellow solid.

LCMS m/z 525.3 (M+H)⁺ (ES⁺).

¹H NMR (DMSO-d₆) δ 13.08 (s, 1H), 8.92 (s, 1H), 6.95 (s, 1H), 3.77-3.60 (m, 2H), 3.19 (q, J=9.9 Hz, 2H), 2.89 (t, J=7.2 Hz, 2H), 2.82 (t, J=7.4 Hz, 4H), 2.75 (td, J=12.8, 2.5 Hz, 2H), 2.64 (t, J=7.3 Hz, 4H), 1.97 (p, J=7.4 Hz, 4H), 1.81-1.67 (m, 2H), 1.65-1.50 (m, 4H), 1.43-1.26 (m, 2H).

### Example 268:5-((1,8-diazaspiro[4.5]decan-8-yl)sulfonyl)-N-(1,2,3,5,6,7-hexahydro-s-indacen-4-yl)-4H-1,2,4-triazol-3-amine

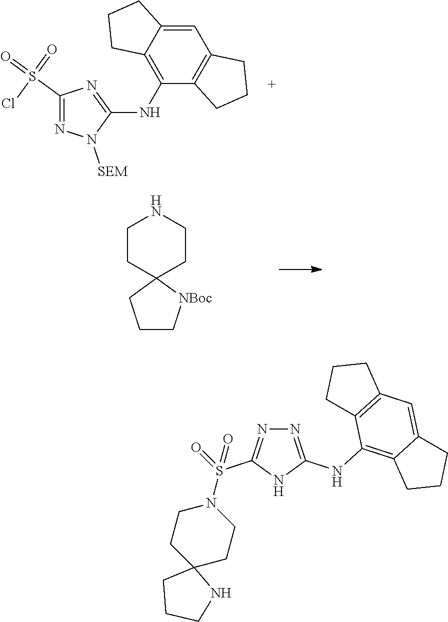 [SMILES: C[Si](C)(C)CCOCn1nc(S(=O)(=O)Cl)nc1Nc1c2c(cc3c1CCC3)CCC2 ; CC(C)(C)OC(=O)N1CCCC12CCNCC2 ; O=S(=O)(c1nnc(Nc2c3c(cc4c2CCC4)CCC3)[nH]1)N1CCC2(CCCN2)CC1]

Prepared according to the general procedure of 5-((6-(2-fluoroethyl)-2,6-diazaspiro[3.4]octan-2-yl)sulfonyl)-N-(1,2,3,5,6,7-hexahydro-s-indacen-4-yl)-4H-1,2,4-triazol-3-amine (Example 239) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and tert-butyl 1,8-diazaspiro[4.5]decane-1-carboxylate to afford the title compound (44 mg, 23%) as a flocculent white solid.

LCMS m/z 443.3 (M+H)⁺ (ES⁺).

¹H NMR (DMSO-d₆) δ 9.27 (s, 1H), 6.94 (s, 1H), 3.37-3.28 (m, 2H), 3.18-3.07 (m, 2H), 2.99 (t, J=7.1 Hz, 2H), 2.82 (t, J=7.4 Hz, 4H), 2.66 (t, J=7.3 Hz, 4H), 1.97 (p, J=7.5 Hz, 4H), 1.79 (p, J=7.2 Hz, 2H), 1.68 (t, J=5.7 Hz, 4H), 1.62-1.55 (m, 2H). Two exchangeable protons not observed.

### Example 269: N-(1,2,3,5,6,7-hexahydro-s-indacen-4-yl)-5-(((1S,5S)-6-methyl-2,6-diazabicyclo[3.2.0]heptan-2-yl)sulfonyl)-4H-1,2,4-triazol-3-amine

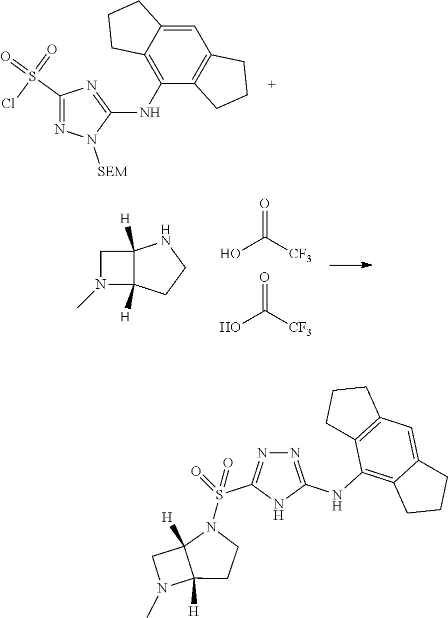

Prepared according to the general procedure of 5-((6-(2-fluoroethyl)-2,6-diazaspiro[3.4]octan-2-yl)sulfonyl)-N-(1,2,3,5,6,7-hexahydro-s-indacen-4-yl)-4H-1,2,4-triazol-3-amine (Example 239) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and (1S,5S)-6-methyl-2,6-diazabicyclo[3.2.0]heptane bis(2,2,2-trifluoroacetate) to afford the title compound (41 mg, 23%) as a flocculent white solid.

LCMS m/z 415.2 (M+H)⁺ (ES⁺).

¹H NMR (DMSO-d₆) δ 8.96 (s, 1H), 6.95 (s, 1H), 3.84 (dd, J=6.8, 4.2 Hz, 1H), 3.54-3.46 (m, 2H), 3.28-3.12 (m, 3H), 3.07-2.94 (m, 2H), 2.82 (t, J=7.4 Hz, 4H), 2.62 (t, J=7.4 Hz, 4H), 2.27 (s, 3H), 1.96 (p, J=7.4 Hz, 4H). One exchangeable proton not observed.

### Example 270:N-(1,2,3,5,6,7-hexahydro-s-indacen-4-yl)-5-((1-methyl-1,7-diazaspiro[3.5]nonan-7-yl)sulfonyl)-4H-1,2,4-triazol-3-amine

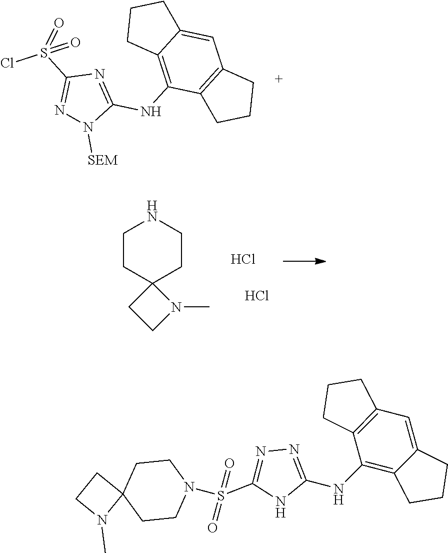

1-Methyl-1,7-diazaspiro[3.5]nonane dihydrochloride (0.098 g, 0.460 mmol) and Et₃N (0.192 mL, 1.38 mmol) were added to a solution of 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) (180 mg, 0.383 mmol) in DCM (4 mL) and the reaction mixture was stirred for 2 h. The reaction mixture was concentrated in vacuo and the resultant residue was dissolved in 4 M HCl in dioxane (4 mL) and stirred at RT for 16 h. The reaction mixture was concentrated in vacuo and the crude product was purified by acidic prep HPLC (20-50% MeOH in water) to afford the title compound (33 mg, 19%) as a flocculent white solid.

LCMS m/z 443.3 (M+H)⁺ (ES⁺).

¹H NMR (DMSO-d₆) δ 9.22 (s, 1H), 6.94 (s, 1H), 3.71-3.61 (m, 2H), 3.52 (t, J=7.6 Hz, 2H), 2.81 (t, J=7.4 Hz, 4H), 2.78-2.70 (m, 2H), 2.64 (t, J=7.3 Hz, 4H), 2.36 (s, 3H), 2.10-1.91 (m, 8H), 1.79 (td, J=12.6, 4.5 Hz, 2H). One exchangeable proton not observed.

### Example 271: 1-((5-((1,2,3,5,6,7-hexahydro-s-indacen-4-yl)amino)-4H-1,2,4-triazol-3-yl)sulfonyl)-N,N-dimethylazepan-4-amine

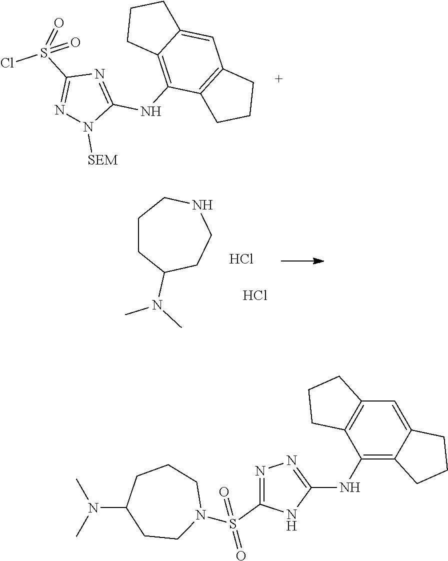

N,N-dimethylazepan-4-amine dihydrochloride (0.142 g, 0.658 mmol) and Et₃N (0.275 mL, 1.97 mmol) were added to a solution of 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) (0.205 g, 0.438 mmol) in DCM (4 mL) and the reaction mixture was stirred for 2 h. The reaction mixture was concentrated in vacuo, the resultant residue was dissolved in 4 M HCl in dioxane (4 mL) and the reaction mixture was stirred at RT for 16 h. The reaction mixture was concentrated in vacuo and the product was purified by acidic prep HPLC (20-50% MeOH in water) to afford the title compound (45 mg, 23%) as a flocculent white solid.

LCMS m/z 445.3 (M+H)⁺ (ES⁺).

¹H NMR (DMSO-d₆) δ 9.01 (s, 1H), 6.95 (s, 1H), 3.54-3.45 (m, 1H), 3.42 (dt, J=13.8, 4.3 Hz, 1H), 3.30-3.14 (m, 2H), 3.11-2.99 (m, 1H), 2.82 (t, J=7.4 Hz, 4H), 2.63 (t, J=7.4 Hz, 4H), 2.48 (s, 6H), 2.13-2.04 (m, 1H), 2.02-1.93 (m, 5H), 1.93-1.85 (m, 1H), 1.79-1.68 (m, 1H), 1.61-1.48 (m, 2H). One exchangeable proton not observed.

### Example 272: 5-((1,2,3,5,6,7-hexahydro-s-indacen-4-yl)amino)-N-(2-hydroxy-2-methylpropyl)-4H-1,2,4-triazole-3-sulfonamide

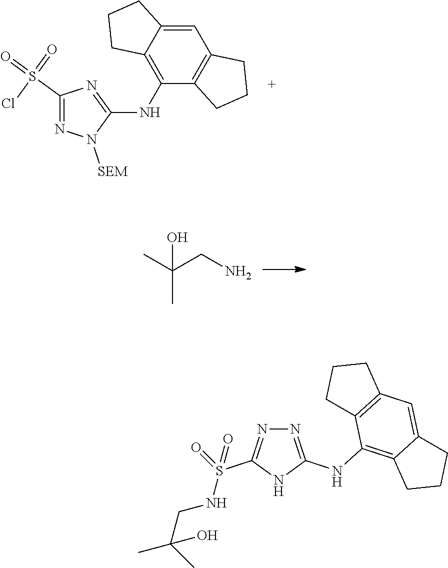

Prepared according to the general procedure of 5-((6-(2-fluoroethyl)-2,6-diazaspiro[3.4]octan-2-yl)sulfonyl)-N-(1,2,3,5,6,7-hexahydro-s-indacen-4-yl)-4H-1,2,4-triazol-3-amine (Example 239) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and 1-amino-2-methylpropan-2-ol to afford the title compound (39 mg, 22%) as a pale yellow solid.

LCMS m/z 392.2 (M+H)⁺ (ES⁺).

¹H NMR (DMSO-d₆) δ 8.84 (s, 1H), 7.68 (t, J=6.4 Hz, 1H), 6.95 (s, 1H), 4.39 (s, 1H), 2.92-2.76 (m, 6H), 2.64 (t, J=7.3 Hz, 4H), 1.97 (p, J=7.4 Hz, 4H), 1.06 (s, 6H). One exchangeable proton not observed.

### Example 273: N-(1,2,3,5,6,7-hexahydro-s-indacen-4-yl)-5-((4-methyl-1,4-diazepan-1-yl)sulfonyl)-4H-1,2,4-triazol-3-amine

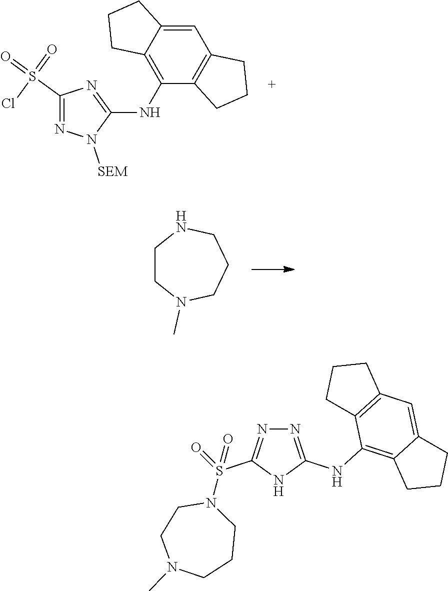

A solution of 5-((1,2,3,5,6,7-hexahydro-s-indacen-4-yl)amino)-1-((2-(trimethylsilyl)-ethoxy)methyl)-1H-1,2,4-triazole-3-sulfonyl chloride (Intermediate B4) (150 mg, 0.320 mmol) in DCM (2 mL) was added to a solution of 1-methyl-1,4-diazepane (0.044 mL, 0.352 mmol), Et₃N (0.067 mL, 0.480 mmol), and DCM (1 mL) and the reaction was stirred for 1 h. The reaction mixture was concentrated in vacuo, HCl (4 M in dioxane) (2 mL, 8.00 mmol) was added and the reaction mixture was stirred at RT overnight. The reaction mixture was concentrated in vacuo and the crude product was purified by acidic prep HPLC (20%-50% MeOH in water) to afford the title compound (12 mg, 9%) as a flocculent white solid.

LCMS m/z 417.5 (M+H)⁺ (ES⁺); 415.2 (M−H)⁻ (ES⁻).

¹H NMR (DMSO-d₆) δ 9.07 (s, 1H), 6.98 (s, 1H), 3.86 (app q, J=8.5 Hz, 4H), 2.84 (t, J=7.4 Hz, 4H), 2.67 (t, J=7.4 Hz, 4H), 2.47-2.40 (m, 4H), 2.20 (s, 3H), 1.99 (p, J=7.4 Hz, 4H), 1.86 (t, J=7.1 Hz, 2H). One exchangeable proton not observed

### Example 274: N-(1,2,3,5,6,7-hexahydro-s-indacen-4-yl)-5-((6-methyl-2,6-diazaspiro[3.4]octan-2-yl)sulfonyl)-4H-1,2,4-triazol-3-amine

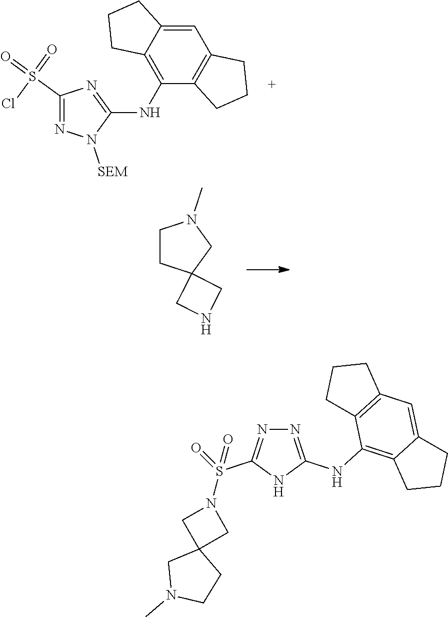

Prepared according to the general procedure of N-(1,2,3,5,6,7-hexahydro-s-indacen-4-yl)-5-((4-methyl-1,4-diazepan-1-yl)sulfonyl)-4H-1,2,4-triazol-3-amine (Example 273) from 5-((1,2,3,5,6,7-hexahydro-s-indacen-4-yl)amino)-1-((2-(trimethylsilyl)ethoxy)-methyl)-1H-1,2,4-triazole-3-sulfonyl chloride (Intermediate B4) and 6-methyl-2,6-diazaspiro[3.4]octane (Intermediate X17) to afford the title compound (7 mg, 5%) as a flocculent white solid.

LCMS m/z 429.3 (M+H)⁺ (ES⁺); 427.5 (M−H)⁻ (ES⁻).

¹H NMR (DMSO-d₆) δ 8.99 (s, 1H), 6.97 (s, 1H), 3.59-3.53 (m, 2H), 3.41 (t, J=6.2 Hz, 2H), 3.17-3.05 (m, 4H), 2.83 (t, J=7.4 Hz, 4H), 2.67-2.60 (m, 7H), 2.03-1.93 (m, 6H). One exchangeable proton not observed.

### Example 275: N-(1,2,3,5,6,7-hexahydro-s-indacen-4-yl)-5-((7-methyl-2,7-diazaspiro[3-5]nonan-2-yl)sulfonyl)-4H-1,2,4-triazol-3-amine

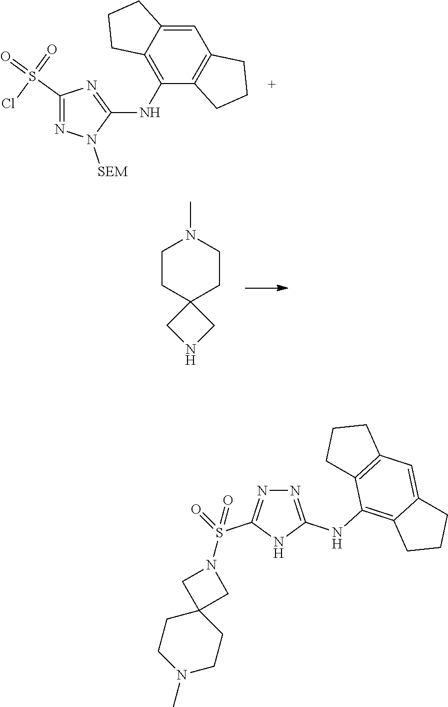

Prepared according to the general procedure of N-(1,2,3,5,6,7-hexahydro-s-indacen-4-yl)-5-((4-methyl-1,4-diazepan-1-yl)sulfonyl)-4H-1,2,4-triazol-3-amine (Example 273) from 5-((1,2,3,5,6,7-hexahydro-s-indacen-4-yl)amino)-1-((2-(trimethylsilyl)ethoxy)-methyl)-1H-1,2,4-triazole-3-sulfonyl chloride (Intermediate B4) and 7-methyl-2,7-diazaspiro[35]nonane (Intermediate X16) to afford the title compound (9 mg, 6%) as a flocculent white solid.

LCMS m/z 443.3 (M+H)⁺ (ES⁺); 441.2 (M−H)⁻ (ES⁻).

¹H NMR (DMSO-d₆) δ 9.12 (s, 1H), 6.98 (s, 1H), 3.66 (s, 4H), 2.84 (t, J=7.4 Hz, 4H), 2.66 (t, J=7.3 Hz, 4H), 2.40 (br s, 4H), 2.26 (s, 3H), 1.98 (p, J=7.4 Hz, 4H), 1.57 (t, J=5.5 Hz, 4H). One exchangeable proton not observed.

### Example 276:N-(1,2,3,5,6,7-hexahydro-s-indacen-4-yl)-5-((2-methyl-2,8-diazaspiro[4.5]decan-8-yl)sulfonyl)-4H-1,2,4-triazol-3-amine

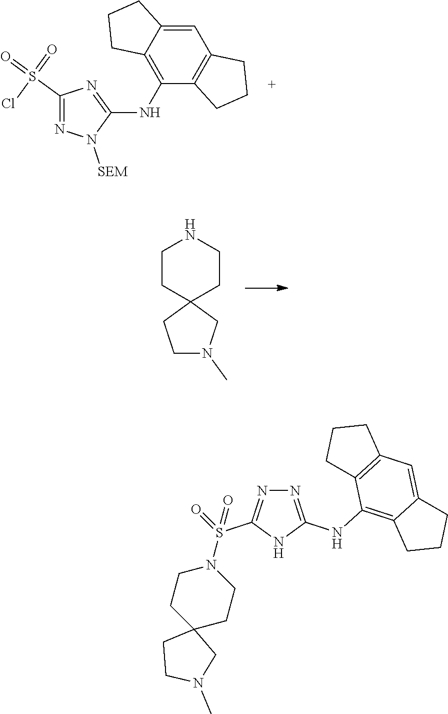 [SMILES: C[Si](C)(C)CCOCn1nc(S(=O)(=O)Cl)nc1Nc1c2c(cc3c1CCC3)CCC2 ; CN1CCC2(CCNCC2)C1 ; CN1CCC2(CCN(S(=O)(=O)c3nnc(Nc4c5c(cc6c4CCC6)CCC5)[nH]3)CC2)C1]

Prepared according to the general procedure of N-(1,2,3,5,6,7-hexahydro-s-indacen-4-yl)-5-((4-methyl-1,4-diazepan-1-yl)sulfonyl)-4H-1,2,4-triazol-3-amine (Example 273) from 5-((1,2,3,5,6,7-hexahydro-s-indacen-4-yl)amino)-1-((2-(trimethylsilyl)ethoxy)-methyl)-1H-1,2,4-triazole-3-sulfonyl chloride (Intermediate B4) and 2-methyl-2,8-diazaspiro[4.5]decane (Intermediate X9) to afford the title compound (36 mg, 24%) as a flocculent white solid.

LCMS m/z 457.3 (M+H)⁺ (ES⁺); 455.2 (M−H)⁻ (ES⁻).

¹H NMR (DMSO-d₆) δ 9.09 (s, 1H), 6.95 (s, 1H), 3.22-3.08 (m, 4H), 2.87-2.77 (m, 6H), 2.65 (t, J=7.3 Hz, 4H), 2.59 (s, 2H), 2.44 (s, 3H), 1.97 (p, J=7.4 Hz, 4H), 1.67-1.54 (m, 6H). One exchangeable proton not observed.

### Example 277:N-(1,2,3,5,6,7-hexahydro-s-indacen-4-yl)-5-((7-methyl-2,7-diazaspiro[4.4]nonan-2-yl)sulfonyl)-4H-1,2,4-triazol-3-amine

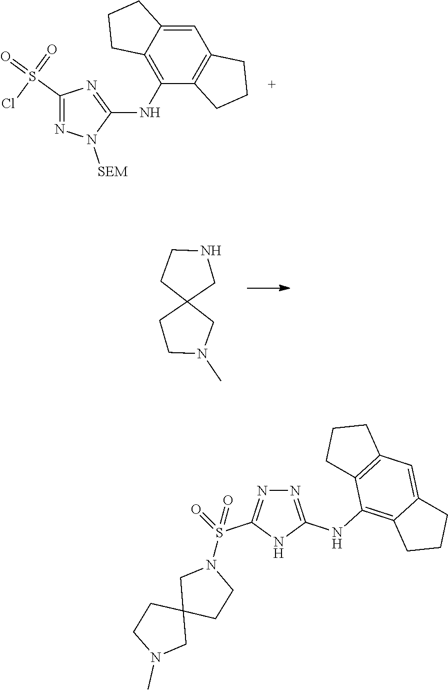

Prepared according to the general procedure of N-(1,2,3,5,6,7-hexahydro-s-indacen-4-yl)-5-((4-methyl-1,4-diazepan-1-yl)sulfonyl)-4H-1,2,4-triazol-3-amine (Example 273) from 5-((1,2,3,5,6,7-hexahydro-s-indacen-4-yl)amino)-1-((2-(trimethylsilyl)ethoxy)-methyl)-1H-1,2,4-triazole-3-sulfonyl chloride (Intermediate B4) and 2-methyl-2,7-diazaspiro[4.4]nonane to afford the title compound (12 mg, 8%) as a flocculent white solid.

LCMS m/z 443.4 (M+H)⁺ (ES⁺); 441.2 (M−H)⁻ (ES⁻).

¹H NMR (DMSO-d₆) δ 8.98 (s, 1H), 6.97 (s, 1H), 3.43-3.33 (m, 2H), 3.30 (d, J=10.0 Hz, 1H), 3.22 (d, J=10.0 Hz, 1H), 2.83 (t, J=7.4 Hz, 4H), 2.70-2.61 (m, 6H), 2.49-2.44 (m, 2H), 2.34 (s, 3H), 1.98 (p, J=7.4 Hz, 4H), 1.90-1.82 (m, 1H), 1.81-1.74 (m, 1H), 1.69-1.59 (m, 2H). One exchangeable proton not observed.

### Example 278: N-(1,2,3,5,6,7-hexahydro-s-indacen-4-yl)-5-((1-methyl-1,8-diazaspiro[4.5]decan-8-yl)sulfonyl)-4H-1,2,4-triazol-3-amine

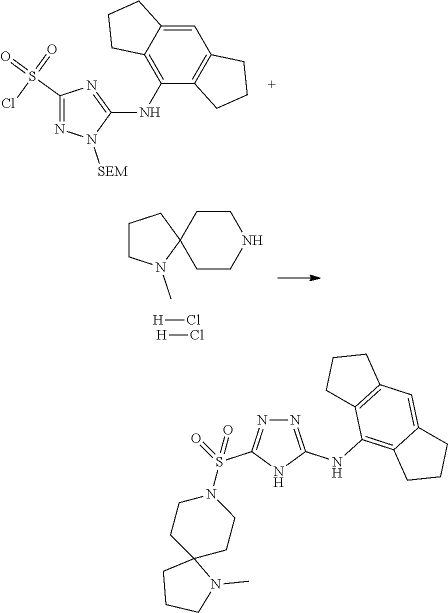

Prepared according to the general procedure of 1-((5-((1,2,3,5,6,7-hexahydro-s-indacen-4-yl)amino)-4H-1,2,4-triazol-3-yl)sulfonyl)-N,N-dimethylazepan-4-amine (Example 271) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B⁴) and 1-methyl-1,8-diazaspiro[4.5]decane dihydrochloride to afford the title compound (17 mg, 12%) as a flocculent white solid.

LCMS m/z 457.0 (M+H)⁺ (ES⁺); 455.2 (M−H)⁻ (ES⁻).

¹H NMR (DMSO-d₆) δ 9.07 (s, 1H), 8.20 (s, 1H), 6.96 (s, 1H), 3.76-3.68 (m, 2H), 2.89-2.76 (m, 8H), 2.65 (t, J=7.3 Hz, 4H), 2.28 (s, 3H), 1.97 (p, J=7.4 Hz, 4H), 1.78-1.70 (m, 2H), 1.70-1.64 (m, 3H), 1.38 (br d, J=12.6 Hz, 2H). One exchangeable proton not observed.

### Example 279: N-(1,2,3,5,6,7-hexahydro-s-indacen-4-yl)-5-((3-((methylamino)methyl)azetidin-1-yl)sulfonyl)-4H-1,2,4-triazol-3-amine

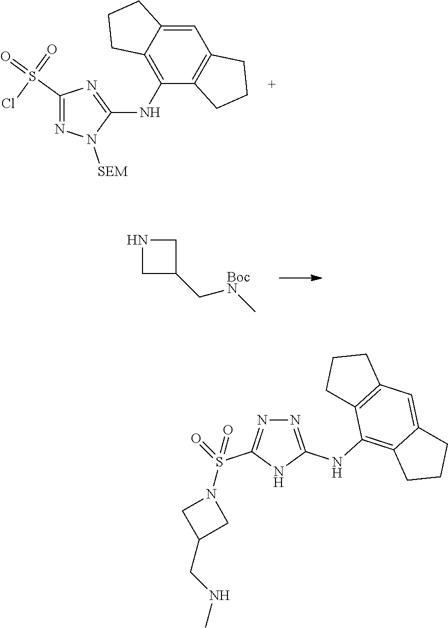 [SMILES: C[Si](C)(C)CCOCn1nc(S(=O)(=O)Cl)nc1Nc1c2c(cc3c1CCC3)CCC2 ; CN(CC1CNC1)C(=O)OC(C)(C)C ; CNCC1CN(S(=O)(=O)c2nnc(Nc3c4c(cc5c3CCC5)CCC4)[nH]2)C1]

Prepared according to the general procedure of 5-((6-(2-fluoroethyl)-2,6-diazaspiro[3.4]octan-2-yl)sulfonyl)-N-(1,2,3,5,6,7-hexahydro-s-indacen-4-yl)-4H-1,2,4-triazol-3-amine (Example 239) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and tert-butyl (azetidin-3-ylmethyl)(methyl)carbamate to afford the title compound (10 mg, 8%) as a flocculent white solid.

LCMS m/z 403.2 (M+H)⁺ (ES⁺).

¹H NMR (DMSO-d₆): δ 9.17 (s, 1H), 6.96 (s, 1H), 3.99 (t, J=8.4 Hz, 2H), 3.71 (dd, J=8.5, 5.8 Hz, 2H), 2.88-2.77 (m, 6H), 2.73-2.62 (m, 5H), 2.40 (s, 3H), 1.98 (p, J=7.4 Hz, 4H). Two exchangeable protons not observed.

### Example 280: N-(1,2,3,5,6,7-hexahydro-s-indacen-4-yl)-5-((2-methyl-2,7-diazaspiro[3.5]nonan-7-yl)sulfonyl)-4H-1,2,4-triazol-3-amine

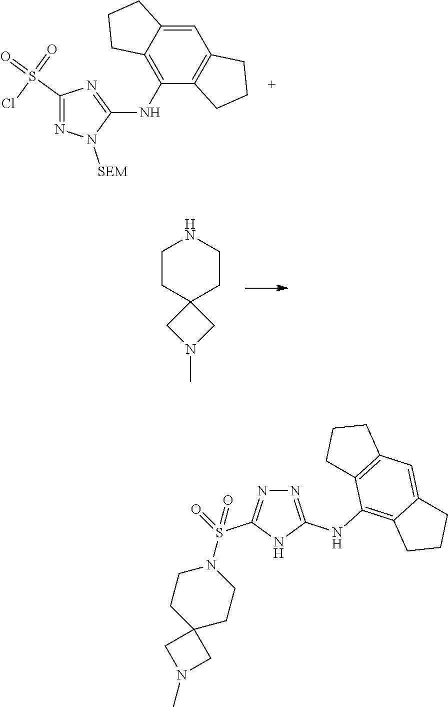

Prepared according to the general procedure of 5-((6-(2-fluoroethyl)-2,6-diazaspiro[3.4]octan-2-yl)sulfonyl)-N-(1,2,3,5,6,7-hexahydro-s-indacen-4-yl)-4H-1,2,4-triazol-3-amine (Example 239) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and 2-methyl-2,7-diazaspiro[3.5]nonane (Intermediate X14) to afford the title compound (17 mg, 12%) as a flocculent white solid.

LCMS m/z 443.3 (M+H)⁺ (ES⁺).

¹H NMR (DMSO-d₆): δ 8.98 (s, 1H), 6.95 (s, 1H), 3.52 (s, 4H), 3.11 (t, J=5.6 Hz, 4H), 2.82 (t, J=7.4 Hz, 4H), 2.66-2.58 (m, 7H), 1.97 (p, J=7.4 Hz, 4H), 1.80 (t, J=5.6 Hz, 4H). One exchangeable proton not observed.

### Example 281: 5-((5,6-dihydroimidazo[1,5-a]pyrazin-7 (8H)-yl)sulfonyl)-N-(1,2,3,5,6,7-hexahydro-s-indacen-4-yl)-4H-1,2,4-triazol-3-amine

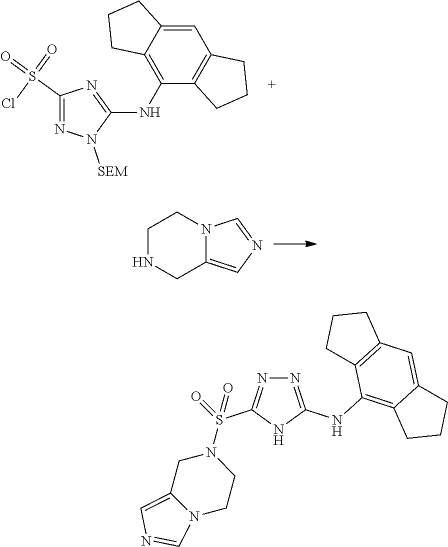 [SMILES: C[Si](C)(C)CCOCn1nc(S(=O)(=O)Cl)nc1Nc1c2c(cc3c1CCC3)CCC2 ; c1ncn2c1CNCC2 ; O=S(=O)(c1nnc(Nc2c3c(cc4c2CCC4)CCC3)[nH]1)N1CCn2cncc2C1]

Prepared according to the general procedure of 5-((6-(2-fluoroethyl)-2,6-diazaspiro[3.4]octan-2-yl)sulfonyl)-N-(1,2,3,5,6,7-hexahydro-s-indacen-4-yl)-4H-1,2,4-triazol-3-amine (Example 239) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and 5,6,7,8-tetrahydroimidazo[1,5-a]pyrazine to afford the title compound (15 mg, 11%) as a yellow solid.

LCMS m/z 426.3 (M+H)⁺ (ES⁺).

¹H NMR (DMSO-d₆): δ 13.23 (s, 1H), 9.00 (s, 1H), 8.60 (br s, 1H), 7.28 (br s, 1H), 6.96 (s, 1H), 4.56 (s, 2H), 4.23 (s, 2H), 3.73 (t, J=5.5 Hz, 2H), 2.81 (t, J=7.4 Hz, 4H), 2.59 (t, J=7.3 Hz, 4H), 1.95 (p, J=7.4 Hz, 4H).

### Example 282: N-(1,2,3,5,6,7-hexahydro-s-indacen-4-yl)-5-((3-(2-methoxypropan-2-yl)pyrrolidin-1-yl)sulfonyl)-4H-1,2,4-triazol-3-amine

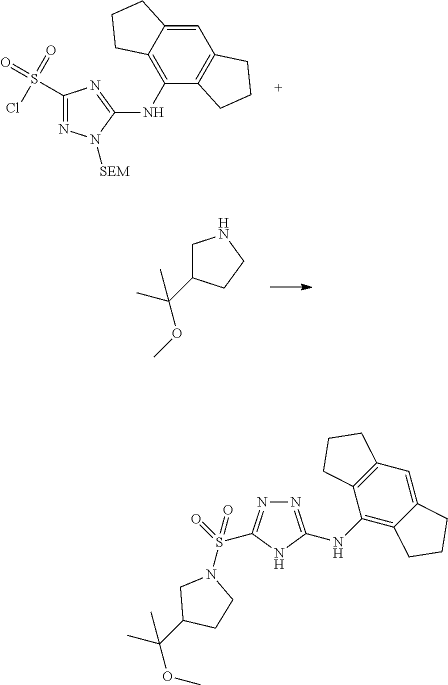

Prepared according to the general procedure of 5-((6-(2-fluoroethyl)-2,6-diazaspiro[3.4]octan-2-yl)sulfonyl)-N-(1,2,3,5,6,7-hexahydro-s-indacen-4-yl)-4H-1,2,4-triazol-3-amine (Example 239) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and 3-(2-methoxypropan-2-yl)pyrrolidine (Intermediate X13) to afford the title compound (26 mg, 18%) as a white solid.

LCMS m/z 446.3 (M+H)⁺ (ES⁺).

¹H NMR (DMSO-d₆): δ 13.04 (s, 1H), 8.91 (s, 1H), 6.96 (s, 1H), 3.49-3.40 (m, 1H), 3.37-3.33 (m, 1H), 3.29-3.22 (m, 1H), 3.11-3.04 (m, 4H), 2.83 (t, J=7.4 Hz, 4H), 2.63 (t, J=7.4 Hz, 4H), 2.28-2.17 (m, 1H), 1.98 (p, J=7.4 Hz, 4H), 1.82-1.73 (m, 1H), 1.68-1.58 (m, 1H), 1.03 (s, 3H), 1.00 (s, 3H).

### Example 283: N-(1,2,3,5,6,7-hexahydro-s-indacen-4-yl)-5-((3-(methoxymethyl)pyrrolidin-1-yl)sulfonyl)-4H-1,2,4-triazol-3-amine

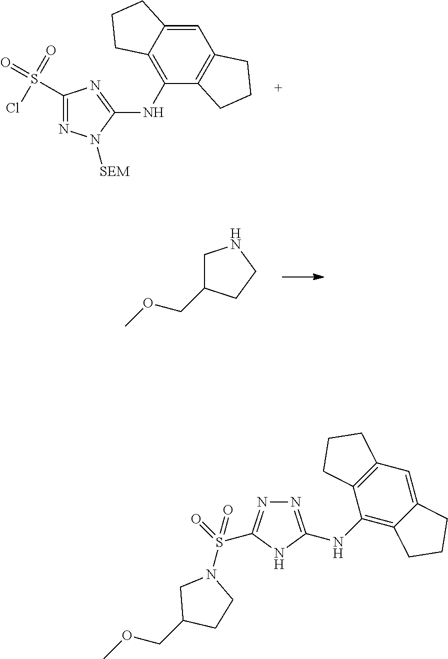

Prepared according to the general procedure of 5-((6-(2-fluoroethyl)-2,6-diazaspiro[3.4]octan-2-yl)sulfonyl)-N-(1,2,3,5,6,7-hexahydro-s-indacen-4-yl)-4H-1,2,4-triazol-3-amine (Example 239) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and 3-(methoxymethyl)pyrrolidine to afford the title compound (11 mg, 12%) as a yellow gum.

LCMS m/z 418.3 (M+H)⁺ (ES⁺).

¹H NMR (DMSO-d₆): δ13.06 (s, 1H), 8.93 (s, 1H), 6.96 (s, 1H), 3.45-3.36 (m, 2H), 3.33-3.27 (m, 1H), 3.23-3.16 (m, 4H), 3.12 (dd, J=9.4, 7.5 Hz, 1H), 3.02 (dd, J=10.2, 6.9 Hz, 1H), 2.82 (t, J=7.4 Hz, 4H), 2.63 (t, J=7.3 Hz, 4H), 2.35 (app sept, J=7.2 Hz, 1H), 1.97 (p, J=7.4 Hz, 4H), 1.91-1.84 (m, 1H), 1.57-1.48 (m, 1H).

### Example 284: (S)-(1-((5-((1,2,3,5,6,7-hexahydro-s-indacen-4-yl)amino)-4H-1,2,4-triazol-3-yl)sulfonyl)pyrrolidin-3-yl)methanol

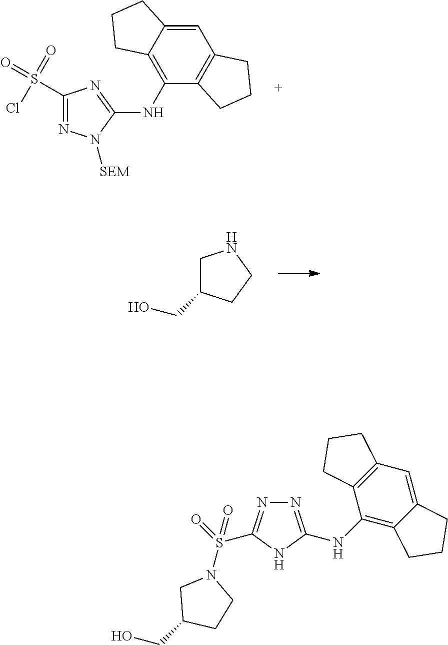 [SMILES: C[Si](C)(C)CCOCn1nc(S(=O)(=O)Cl)nc1Nc1c2c(cc3c1CCC3)CCC2 ; OC[C@H]1CCNC1 ; O=S(=O)(c1nnc(Nc2c3c(cc4c2CCC4)CCC3)[nH]1)N1CC[C@H](CO)C1]

Prepared according to the general procedure of 5-((6-(2-fluoroethyl)-2,6-diazaspiro[3.4]octan-2-yl)sulfonyl)-N-(1,2,3,5,6,7-hexahydro-s-indacen-4-yl)-4H-1,2,4-triazol-3-amine (Example 239) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and (S)-pyrrolidin-3-ylmethanol to afford the title compound (14 mg, 15%) as a yellow gum.

LCMS m/z 404.2 (M+H)⁺ (ES⁺).

¹H NMR (DMSO-d₆): δ 13.02 (s, 1H), 8.90 (s, 1H), 6.95 (s, 1H), 4.67 (t, J=5.3 Hz, 1H), 3.45-3.35 (m, 2H), 3.31-3.22 (m, 3H), 3.02 (dd, J=10.1, 7.1 Hz, 1H), 2.82 (t, J=7.4 Hz, 4H), 2.63 (t, J=7.3 Hz, 4H), 2.23 (app sept, J=7.2 Hz, 1H), 1.97 (p, J=7.4 Hz, 4H), 1.88-1.79 (m, 1H), 1.59-1.49 (m, 1H).

### Example 285: N-(1,2,3,5,6,7-hexahydro-s-indacen-4-yl)-5-((3-(pyridin-2-yl)azetidin-1-yl)sulfonyl)-4H-1,2,4-triazol-3-amine

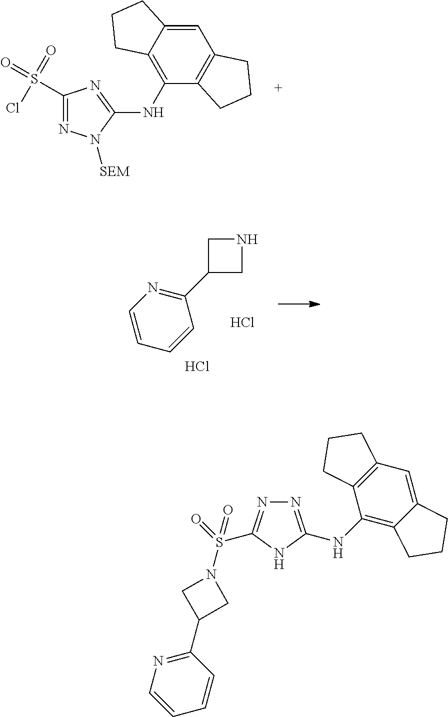 [SMILES: C[Si](C)(C)CCOCn1nc(S(=O)(=O)Cl)nc1Nc1c2c(cc3c1CCC3)CCC2 ; c1ccc(C2CNC2)nc1 ; Cl ; Cl ; O=S(=O)(c1nnc(Nc2c3c(cc4c2CCC4)CCC3)[nH]1)N1CC(c2ccccn2)C1]

To a stirred solution of 2-(azetidin-3-yl)pyridine dihydrochloride (0.066 g, 0.320 mmol) and Et₃N (0.090 mL, 0.640 mmol) in DCM (1 mL) was added 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) (0.1 g, 0.213 mmol) in DCM (4 mL) and the reaction was stirred at RT for 2 h. The reaction mixture was concentrated in vacuo and 4 M HCl in dioxane (3 mL) was added. The solution was stirred at RT for 16 h then concentrated in vacuo and the crude product was purified by acidic prep HPLC (35-65% MeOH in water) to afford the title compound (10 mg, 11%) as a flocculent white solid.

LCMS m/z 437.3 (M+H)⁺ (ES⁺).

¹H NMR (DMSO-d₆). δ 13.18 (s, 1H), 8.97 (s, 1H), 8.49-8.47 (m, 1H), 7.71 (td, J=7.7, 1.8 Hz, 1H), 7.29-7.19 (m, 2H), 6.95 (s, 1H), 4.21 (t, J=8.4 Hz, 2H), 4.16 (t, J=7.6 Hz, 2H), 3.97-3.90 (m, 1H), 2.81 (t, J=7.4 Hz, 4H), 2.60 (t, J=7.3 Hz, 4H), 1.93 (p, J=7.4 Hz, 4H).

### Example 286: 1-(1-((5-((1,2,3,5,6,7-hexahydro-s-indacen-4-yl)oxy)-4H-1,2,4-triazol-3-yl)sulfonyl)pyrrolidin-3-yl)-N,N-dimethylmethanamine

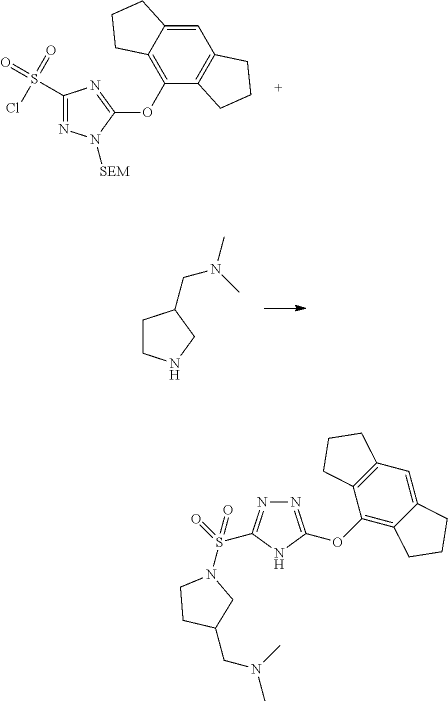

Prepared according to the general procedure of N-(1,2,3,5,6,7-hexahydro-s-indacen-4-yl)-5-((4-methyl-1,4-diazepan-1-yl)sulfonyl)-4H-1,2,4-triazol-3-amine (Example 273) from 5-((1,2,3,5,6,7-hexahydro-s-indacen-4-yl)oxy)-1-((2-(trimethylsilyl)ethoxy)-methyl)-1H-1,2,4-triazole-3-sulfonyl chloride (Intermediate B53) and N,N-dimethyl-1-(pyrrolidin-3-yl)methanamine to afford the title compound (30 mg, 22%) as a white solid.

LCMS m/z 432.3 (M+H)⁺ (ES⁺); 430.3 (M−H)⁻ (ES⁻).

¹H NMR (DMSO-d₆) δ 6.98 (s, 1H), 3.47 (dd, J=10.2, 7.2 Hz, 1H), 3.38-3.30 (m, 1H), 3.27-3.19 (m, 1H), 2.97 (dd, J=10.3, 7.5 Hz, 1H), 2.84 (t, J=7.4 Hz, 4H), 2.61 (t, J=7.4 Hz, 4H), 2.48-2.43 (m, 2H), 2.38-2.27 (m, 7H), 1.99 (p, J=7.4 Hz, 4H), 1.94-1.84 (m, 1H), 1.52-1.37 (m, 1H). One exchangeable proton not observed.

### Example 287:5-((2,7-diazaspiro[35]nonan-7-yl)sulfonyl)-N-(1,2,3,5,6,7-hexahydro-s-indacen-4-yl)-4H-1,2,4-triazol-3-amine

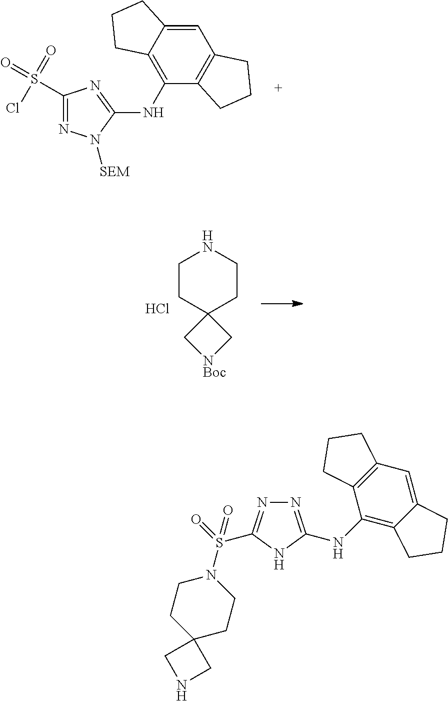 [SMILES: C[Si](C)(C)CCOCn1nc(S(=O)(=O)Cl)nc1Nc1c2c(cc3c1CCC3)CCC2 ; Cl ; CC(C)(C)OC(=O)N1CC2(CCNCC2)C1 ; O=S(=O)(c1nnc(Nc2c3c(cc4c2CCC4)CCC3)[nH]1)N1CCC2(CC1)CNC2]

Prepared according to the general procedure of 5-((6-(2-fluoroethyl)-2,6-diazaspiro[3.4]octan-2-yl)sulfonyl)-N-(1,2,3,5,6,7-hexahydro-s-indacen-4-yl)-4H-1,2,4-triazol-3-amine (Example 239) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and tert-butyl 2,7-diazaspiro[3.5]nonane-2-carboxylate hydrochloride to afford the title compound (0.02 g, 22%) as a flocculent white solid.

LCMS m/z 429.0 (M+H)⁺ (ES⁺).

¹H NMR (DMSO-d₆): δ 9.46 (s, 1H), 8.36 (s, 1H), 6.92 (s, 1H), 3.64 (s, 4H), 3.11 (t, J=5.6 Hz, 4H), 2.81 (t, J=7.4 Hz, 4H), 2.65 (t, J=7.3 Hz, 4H), 1.96 (p, J=7.4 Hz, 4H), 1.82 (t, J=5.7 Hz, 4H). One exchangeable proton not observed.

### Example 288: (R)—N-(1,2,3,5,6,7-hexahydro-s-indacen-4-yl)-5-((3-((methylamino)methyl)pyrrolidin-1-yl)sulfonyl)-4H-1,2,4-triazol-3-amine

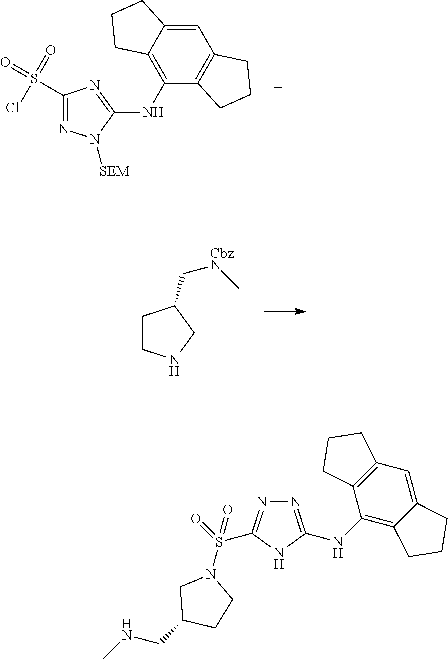 [SMILES: C[Si](C)(C)CCOCn1nc(S(=O)(=O)Cl)nc1Nc1c2c(cc3c1CCC3)CCC2 ; CN(C[C@H]1CCNC1)C(=O)OCc1ccccc1 ; CNC[C@H]1CCN(S(=O)(=O)c2nnc(Nc3c4c(cc5c3CCC5)CCC4)[nH]2)C1]

Step A: Prepared according to the general procedure of 5-((6-(2-fluoroethyl)-2,6-diazaspiro[3.4]octan-2-yl)sulfonyl)-N-(1,2,3,5,6,7-hexahydro-s-indacen-4-yl)-4H-1,2,4-triazol-3-amine (Example 239) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and (S)-benzyl methyl(pyrrolidin-3-ylmethyl)carbamate (Intermediate X18) to afford (R)-benzyl ((1-((5-((1,2,3,5,6,7-hexahydro-s-indacen-4-yl)amino)-4H-1,2,4-triazol-3-yl)sulfonyl)pyrrolidin-3-yl)methyl)(methyl)carbamate as a white solid. The product was taken on to the next step without further purification or analysis.

Step B: 10% Palladium on carbon (3 mg, 0.021 mmol) was placed under an atmosphere of N₂ and a solution of (R)-benzyl ((1-((5-((1,2,3,5,6,7-hexahydro-s-indacen-4-yl)-amino)-4H-1,2,4-triazol-3-yl)sulfonyl)pyrrolidin-3-yl)methyl)(methyl)carbamate (step A) (0.117 g, 0.213 mmol) in EtOH (2 mL) was added. The reaction mixture was stirred under an atmosphere of H₂ at 1 bar pressure for 16 h and then at 5 bar pressure for a further 22 h. The reaction mixture was filtered through Celite, concentrated in vacuo and purified by acidic prep HPLC (20-50% MeOH in water) to afford the title compound (6 mg, 5%) as a flocculent white solid.

LCMS m/z 417.3 (M+H)⁺ (ES⁺).

¹H NMR (DMSO-d₆) δ 9.15 (s, 1H), 6.94 (s, 1H), 3.51-3.45 (m, 1H), 3.45-3.36 (m, 1H), 3.35-3.27 (m, 1H), 3.02 (t, J=8.7 Hz, 1H), 2.82 (t, J=7.4 Hz, 4H), 2.65 (t, J=7.3 Hz, 4H), 2.40-2.25 (m, 5H), 2.04-1.88 (m, 6H), 1.60-1.46 (m, 2H). One exchangeable proton not observed.

### Example 289: 2-(1-((5-((1,2,3,5,6,7-hexahydro-s-indacen-4-yl)amino)-4H-1,2,4-triazol-3-yl)sulfonyl)pyrrolidin-3-yl)propan-2-ol

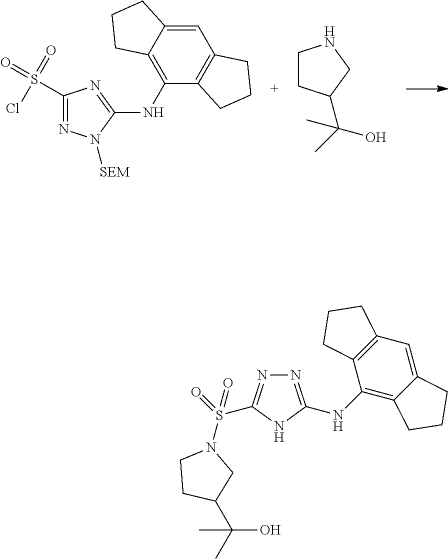

Prepared according to the general procedure of 5-((6-(2-fluoroethyl)-2,6-diazaspiro[3.4]octan-2-yl)sulfonyl)-N-(1,2,3,5,6,7-hexahydro-s-indacen-4-yl)-4H-1,2,4-triazol-3-amine (Example 239) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and 2-(pyrrolidin-3-yl)propan-2-ol to afford the title compound (8 mg, 9%) as a yellow solid.

LCMS m/z 432.3 (M+H)⁺ (ES⁺).

¹H NMR (DMSO-d₆) δ 8.92 (s, 1H), 6.95 (s, 1H), 4.36 (s, 1H), 3.47-3.40 (m, 1H), 3.38-3.30 (m, 1H), 3.29-3.20 (m, 1H), 3.10 (t, J=10.0 Hz, 1H), 2.82 (t, J=7.4 Hz, 4H), 2.63 (t, J=7.3 Hz, 4H), 2.09-2.01 (m, 1H), 1.97 (p, J=7.4 Hz, 4H), 1.81-1.73 (m, 1H), 1.72-1.62 (m, 1H), 1.01 (s, 6H). One exchangeable proton not observed.

### Example 20: N-(2-(dimethylamino)propyl)-5-((1,2,3,5,6,7-hexahydro-s-indacen-4-yl)amino)-4H-1,2,4-triazole-3-sulfonamide

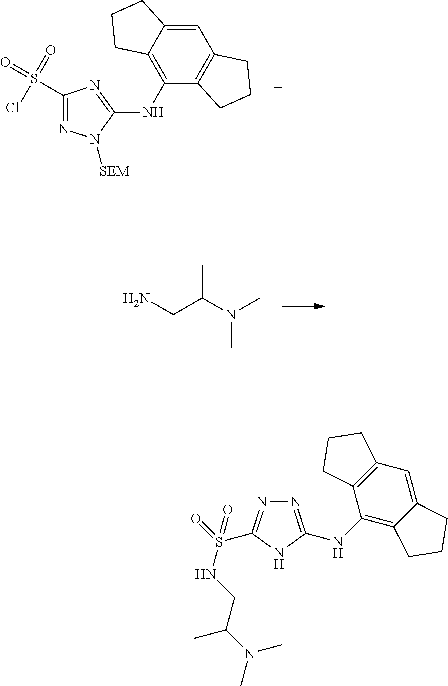

Prepared according to the general procedure of 5-((1,2,3,5,6,7-hexahydro-s-indacen-4-yl)amino)-N-isopentyl-1H-1,2,4-triazole-3-sulfonamide (Example 82) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and N²,N²-dimethylpropane-1,2-diamine to afford the title compound (19 mg, 22%) as a flocculent white solid. LCMS m/z 405.3 (M+H)⁺ (ES⁺).

¹H NMR (DMSO-d₆) δ 8.88 (s, 1H), 6.96 (s, 1H), 3.11 (dd, J=13.3, 6.5 Hz, 1H), 2.98-2.76 (m, 7H), 2.64 (t, J=7.4 Hz, 4H), 2.30 (s, 6H), 1.98 (p, J=7.4 Hz, 4H), 0.97 (d, J=6.5 Hz, 3H). One exchangeable proton not observed.

### Example 211: 5-((3-((dimethylamino)methyl)azetidin-1-yl)sulfonyl)-N-(1,2,3,5,6,7-hexahydro-s-indacen-4-yl)-4H-1,2,4-triazol-3-amine

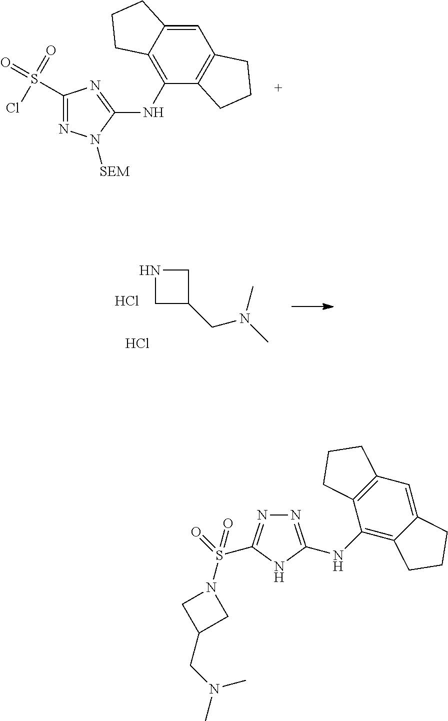

To a stirred solution of 1-(azetidin-3-yl)-N,N-dimethylmethanamine dihydrochloride (0.060 g, 0.320 mmol) and Et₃N (0.135 mL, 0.96 mmol) in DCM (1 mL) was added 5-((1,2,3,5,6,7-hexahydro-s-indacen-4-yl)amino)-1-((2-(trimethylsilyl)ethoxy)methyl)-1H-1,2,4-triazole-3-sulfonyl chloride (0.1 g, 0.213 mmol) (Intermediate B4) in DCM (4 mL) and the reaction was stirred at RT for 18 h. The reaction mixture was concentrated in vacuo and then TFA/DCM (1:1, 4 mL) was added and the reaction stirred at RT for 1 h. The reaction mixture was concentrated in vacuo and the resulting crude product was purified by acidic prep HPLC (20-50% MeOH in water) to afford the title compound (14 mg, 16%) as a flocculent white solid.

LCMS m/z 417.3 (M+H)⁺ (ES⁺).

¹H NMR (DMSO-d₆) δ 9.03 (s, 1H), 6.98 (s, 1H), 4.01 (t, J=8.4 Hz, 2H), 3.66 (dd, J=8.4, 6.1 Hz, 2H), 2.83 (t, J=7.4 Hz, 4H), 2.79-2.73 (m, 1H), 2.71-2.59 (m, 4H), 2.63-2.55 (m, 2H), 2.30 (s, 6H), 1.98 (p, J=7.4 Hz, 4H). One exchangeable proton not observed.

### Example 202: 1-((5-((1,2,3,5,6,7-hexahydro-s-indacen-4-yl)amino)-4H-1,2,4-triazol-3-yl)sulfonyl)-4-methylpiperidin-4-ol

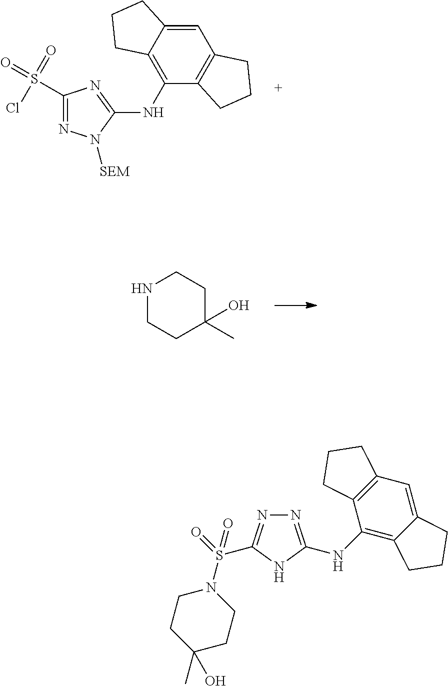

Prepared according to the general procedure of 5-((1,2,3,5,6,7-hexahydro-s-indacen-4-yl)amino)-N-isopentyl-1H-1,2,4-triazole-3-sulfonamide (Example 82) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and 4-methylpiperidin-4-ol to afford the title compound (18 mg, 11%) as a pale yellow solid.

LCMS m/z 418.2 (M+H)⁺ (ES⁺); 416.2 (M−H)⁻ (ES⁻).

¹H NMR (DMSO-d₆) δ 13.02 (s, 1H), 8.92 (s, 1H), 6.95 (s, 1H), 4.31 (s, 1H), 3.38-3.33 (m, 2H), 3.08-3.00 (m, 2H), 2.82 (t, J=7.4 Hz, 4H), 2.63 (t, J=7.3 Hz, 4H), 1.97 (p, J=7.4 Hz, 4H), 1.53-1.44 (m, 4H), 1.10 (s, 3H).

### Example 23: 2-(1-((5-((1,2,3,5,6,7-hexahydro-s-indacen-4-yl)amino)-4H-1,2,4-triazol-3-yl)sulfonyl)piperidin-4-yl)propan-2-ol

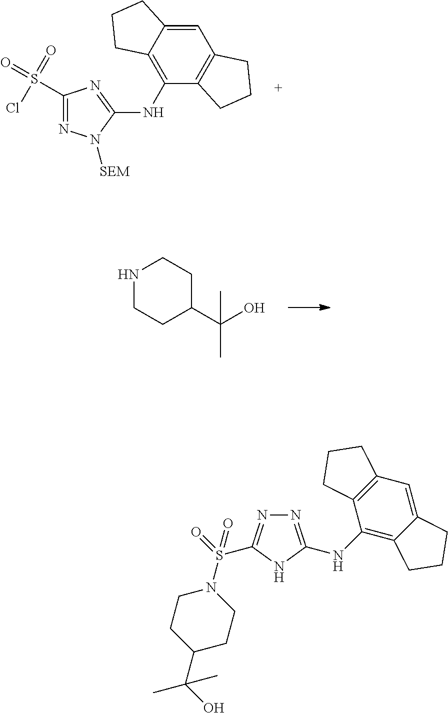

Prepared according to the general procedure of 5-((1,2,3,5,6,7-hexahydro-s-indacen-4-yl)amino)-N-isopentyl-1H-1,2,4-triazole-3-sulfonamide (Example 82) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and 2-(piperidin-4-yl)propan-2-ol to afford the title compound (19 mg, 11%) as a pale yellow solid.

LCMS m/z 446.3 (M+H)⁺ (ES⁺); 444.3 (M−H)⁻ (ES⁻).

¹H NMR (DMSO-d₆) δ 13.01 (s, 1H), 8.93 (s, 1H), 6.96 (s, 1H), 4.16 (s, 1H), 3.74 (d, J=11.5 Hz, 2H), 2.83 (t, J=7.4 Hz, 4H), 2.67-2.59 (m, 6H), 1.98 (p, J=7.4 Hz, 4H), 1.75 (d, J=9.0 Hz, 2H), 1.27-1.17 (m, 3H), 1.01 (s, 6H).

### Example 294: 3-((5-((1,2,3,5,6,7-hexahydro-s-indacen-4-yl)amino)-4H-1,2,4-triazol-3-yl)sulfonyl)-7-methyl-3,7,11-triazaspiro[5.6]dodecan-12-one

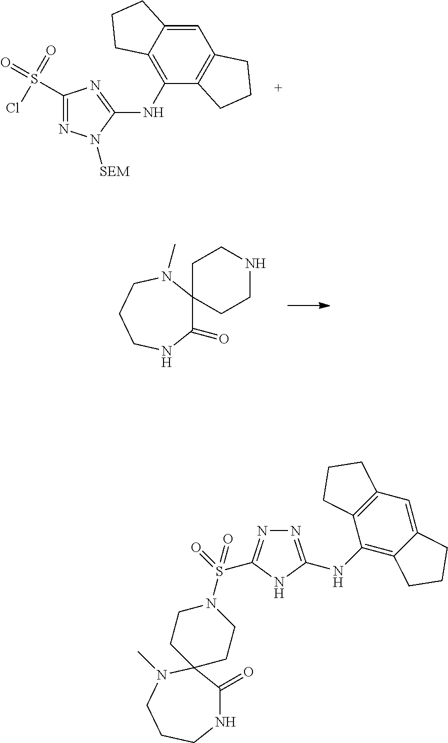 [SMILES: C[Si](C)(C)CCOCn1nc(S(=O)(=O)Cl)nc1Nc1c2c(cc3c1CCC3)CCC2 ; CN1CCCNC(=O)C12CCNCC2 ; CN1CCCNC(=O)C12CCN(S(=O)(=O)c1nnc(Nc3c4c(cc5c3CCC5)CCC4)[nH]1)CC2]

Prepared according to the general procedure of 5-((6-(2-fluoroethyl)-2,6-diazaspiro[3.4]octan-2-yl)sulfonyl)-N-(1,2,3,5,6,7-hexahydro-s-indacen-4-yl)-4H-1,2,4-triazol-3-amine (Example 239) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and 7-methyl-3,7,11-triazaspiro[5.6]dodecan-12-one to afford the title compound (25 mg, 11%) as a white solid.

LCMS m/z 500.4 (M+H)⁺ (ES⁺).

¹H NMR (DMSO-d₆) δ 13.68-11.67 (m, 1H), 8.90 (s, 1H), 7.49 (t, J=5.4 Hz, 1H), 6.95 (s, 1H), 3.37-3.24 (m, 2H), 3.17-2.92 (m, 6H), 2.82 (t, J=7.4 Hz, 4H), 2.63 (t, J=7.3 Hz, 4H), 2.20 (s, 3H), 2.16-2.01 (m, 2H), 1.98 (p, J=7.4 Hz, 4H), 1.88-1.68 (m, 2H), 1.56 (br s, 2H).

### Example 295: N-(1,2,3,5,6,7-hexahydro-s-indacen-4-yl)-5-((8-methyl-5-oxa-2,8-diazaspiro[3-5]nonan-2-yl)sulfonyl)-4H-1,2,4-triazol-3-amine

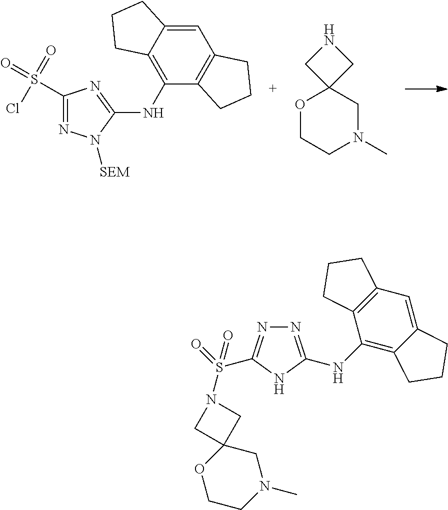

Prepared according to the general procedure of 5-((1,2,3,5,6,7-hexahydro-s-indacen-4-yl)amino)-N-isopentyl-1H-1,2,4-triazole-3-sulfonamide (Example 82) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and 8-methyl-5-oxa-2,8-diazaspiro[3.5]nonane (Intermediate X22) to afford the title compound (21 mg, 10%) as a pale yellow solid.

LCMS m/z 445.3 (M+H)⁺ (ES⁺); 443.2 (M−H)⁻ (ES⁻).

¹H NMR (DMSO-d₆) δ 13.28 (br s, 1H), 9.03 (s, 1H), 6.98 (s, 1H), 3.86 (s, 2H), 3.79 (d, J=9.0 Hz, 2H), 3.55 (s, 2H), 2.83 (t, J=7.4 Hz, 4H), 2.65 (t, J=7.3 Hz, 4H), 2.40-2.18 (m, 4H), 1.98 (p, J=7.4 Hz, 4H). Three protons obscured by solvent.

### Example 296: N-(1,2,3,5,6,7-hexahydro-s-indacen-4-yl)-5-((1-methyl-4-oxa-1,9-diazaspiro[5.5]undecan-9-yl)sulfonyl)-4H-1,2,4-triazol-3-amine

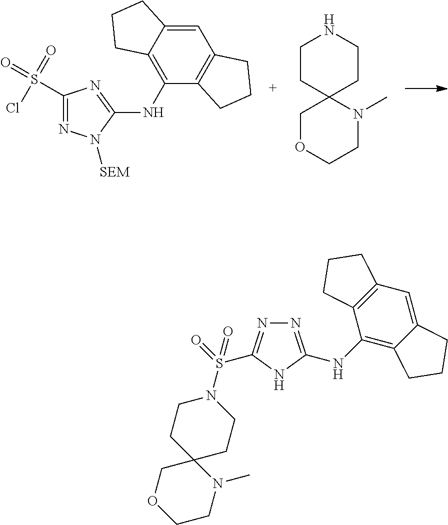 [SMILES: C[Si](C)(C)CCOCn1nc(S(=O)(=O)Cl)nc1Nc1c2c(cc3c1CCC3)CCC2 ; CN1CCOCC12CCNCC2 ; CN1CCOCC12CCN(S(=O)(=O)c1nnc(Nc3c4c(cc5c3CCC5)CCC4)[nH]1)CC2]

Prepared according to the general procedure of 5-((1,2,3,5,6,7-hexahydro-s-indacen-4-yl)amino)-N-isopentyl-1H-1,2,4-triazole-3-sulfonamide (Example 82) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and 1-methyl-4-oxa-1,9-diazaspiro[5-5]undecane (Intermediate X21) to afford the title compound (46 mg, 20%) as a pale yellow solid.

LCMS m/z 473.3 (M+H)⁺ (ES⁺); 471.2 (M−H)⁻ (ES⁻).

¹H NMR (DMSO-d₆) δ 13.15 (br s, 1H), 8.98 (s, 1H), 6.96 (s, 1H), 3.84-3.59 (m, 4H), 3.10-2.94 (m, 2H), 2.82 (t, J=7.4 Hz, 4H), 2.68-2.57 (m, 6H), 2.24-1.51 (m, 8H).

Five protons obscured by solvent.

### Example 297: 5-((4,4-difluoropiperidin-1-yl)sulfonyl)-N-(1,2,3,5,6,7-hexahydro-s-indacen-4-yl)-4H-1,2,4-triazol-3-amine

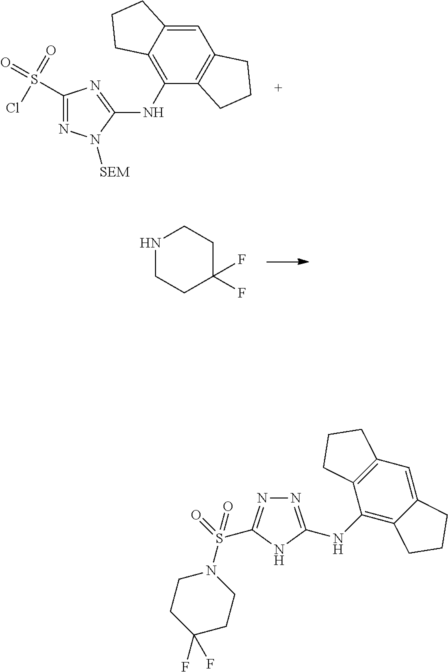 [SMILES: C[Si](C)(C)CCOCn1nc(S(=O)(=O)Cl)nc1Nc1c2c(cc3c1CCC3)CCC2 ; FC1(F)CCNCC1 ; O=S(=O)(c1nnc(Nc2c3c(cc4c2CCC4)CCC3)[nH]1)N1CCC(F)(F)CC1]

Prepared according to the general procedure of 5-((1,2,3,5,6,7-hexahydro-s-indacen-4-yl)amino)-N-isopentyl-1H-1,2,4-triazole-3-sulfonamide (Example 82) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and 4,4-difluoropiperidine to afford the title compound (11 mg, 6%) as a white solid.

LCMS m/z 424.2 (M+H)⁺ (ES⁺); 422.2 (M−H)⁻ (ES⁻).

¹H NMR (DMSO-d₆) δ 13.16 (br s, 1H), 8.98 (s, 1H), 6.96 (s, 1H), 3.39-3.33 (m, 4H), 2.82 (t, J=7.4 Hz, 4H), 2.62 (t, J=7.3 Hz, 4H), 2.13-2.01 (m, 4H), 1.97 (p, J=7.4 Hz, 4H).

### Example 298: 4-((5-((1,2,3,5,6,7-hexahydro-s-indacen-4-yl)amino)-4H-1,2,4-triazol-3-yl)sulfonyl)thiomorpholine 1,1-dioxide

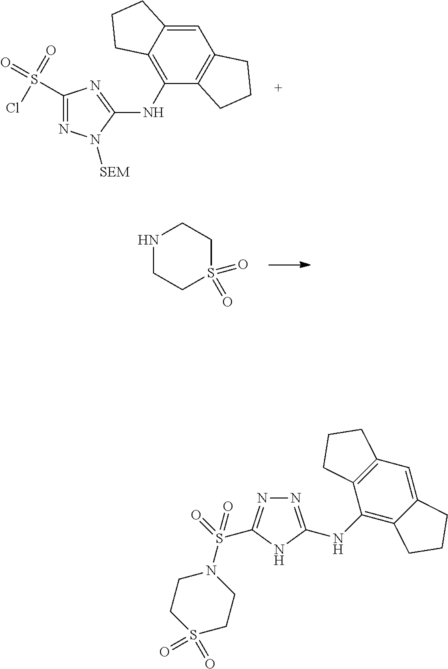 [SMILES: C[Si](C)(C)CCOCn1nc(S(=O)(=O)Cl)nc1Nc1c2c(cc3c1CCC3)CCC2 ; O=S1(=O)CCNCC1 ; O=S1(=O)CCN(S(=O)(=O)c2nnc(Nc3c4c(cc5c3CCC5)CCC4)[nH]2)CC1]

Prepared according to the general procedure of 5-((1,2,3,5,6,7-hexahydro-s-indacen-4-yl)amino)-N-isopentyl-1H-1,2,4-triazole-3-sulfonamide (Example 82) from 5-((1,2,3,5,6,7-hexahydro-s-indacen-4-yl)amino)-1-((2-(trimethylsilyl)ethoxy)methyl)-1H-1,2,4-triazole-3-sulfonyl chloride (Intermediate B4) and thiomorpholine 1,1-dioxide to afford the title compound (33 mg, 16%) as a pale yellow solid.

LCMS m/z 438.2 (M+H)⁺ (ES⁺); 436.2 (M−H)⁻ (ES⁻).

¹H NMR (DMSO-d₆) δ 13.17 (br s, 1H), 9.05 (s, 1H), 6.97 (s, 1H), 3.77-3.65 (m, 4H), 3.33-3.23 (m, 4H), 2.83 (t, J=7.3 Hz, 4H), 2.64 (t, J=7.5 Hz, 4H), 2.13-1.91 (m, 4H).

### Example 299: N-(7-fluoro-5-(2-methoxypyridin-4-yl)-2,3-dihydro-1H-inden-4-yl)-5-((6-methyl-2,6-diazaspiro[3.4]octan-2-yl)sulfonyl)-4H-1,2,4-triazol-3-amine

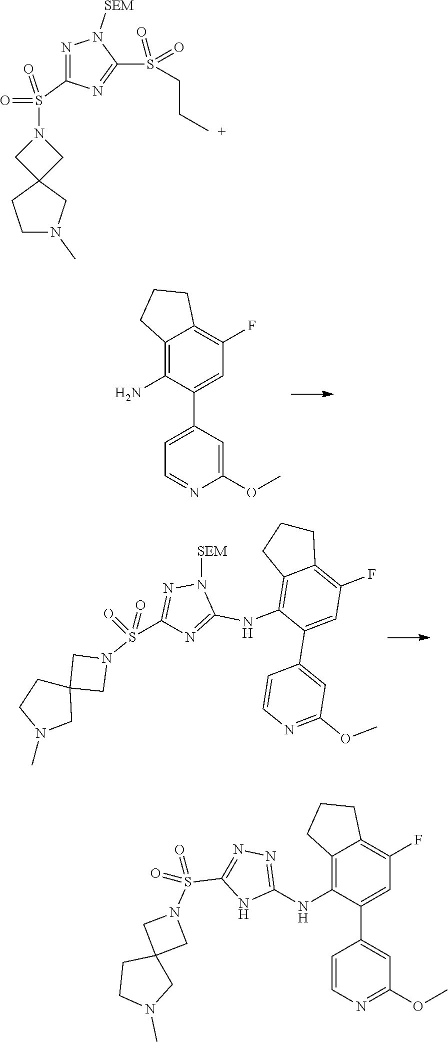

Step A: LiHMDS (1 M in THF, 0.535 mL, 0.535 mmol) was added to 6-methyl-2-((5-(propylsulfonyl)-1-((2-(trimethylsilyl)ethoxy)methyl)-1H-1,2,4-triazol-3-yl)sulfonyl)-2,6-diazaspiro[3.4]octane (Intermediate D1) (132.1 mg, 0.268 mmol) and 7-fluoro-5-(2-methoxypyridin-4-yl)-2,3-dihydro-1H-inden-4-amine (Intermediate C10)(69.1 mg, 0.268 mmol) in THF (2 mL) under N₂. The reaction was stirred at RT for 16 h, quenched with sat aq NH₄Cl (15 mL) and extracted with EtOAc (3×30 mL). The combined organics were dried (phase separator) and concentrated in vacuo. The crude products were purified by chromatography on silica gel (12 g column, 0-10% MeOH/DCM) to afford N-(7-fluoro-5-(2-methoxypyridin-4-yl)-2,3-dihydro-1H-inden-4-yl)-3-((6-methyl-2,6-diazaspiro[3.4]octan-2-yl)sulfonyl)-1-((2-(trimethylsilyl)-ethoxy)methyl)-1H-1,2,4-triazol-5-amine (17.6 mg, 9%) as a pale pink oil.

LCMS m/z 644.5 (M+H)⁺ (ES⁺).

Step B: N-(7-fluoro-5-(2-methoxypyridin-4-yl)-2,3-dihydro-1H-inden-4-yl)-3-((6-methyl-2,6-diazaspiro[3.4]octan-2-yl)sulfonyl)-1-((2-(trimethylsilyl)ethoxy)methyl)-1H-1,2,4-triazol-5-amine (17.6 mg, 0.025 mmol) was dissolved in TFA (1 mL) and stirred at RT for 1 h. The reaction mixture was concentrated in vacuo and the residue was loaded onto a column of SCX (˜0.5 g) in MeOH (1 mL). The column was washed with MeOH (20 mL) and the product was eluted with 0.7 M NH₃ in MeOH (20 mL). The solvents were evaporated to afford the title compound (10 mg, 68%) as a pale yellow solid.

LCMS m/z 514.2 (M+H)⁺ (ES⁺); 512.1 (M−H)⁻ (ES⁻).

¹H NMR (DMSO-d₆) δ 13.09 (s, 1H), 9.00 (s, 1H), 8.14 (dd, J=5.3, 0.7 Hz, 1H), 7.13 (d, J=9.1 Hz, 1H), 6.96 (dd, J=5.3, 1.5 Hz, 1H), 6.79-6.78 (m, 1H), 3.84 (s, 3H), 3.80 (d, J=8.4 Hz, 2H), 3.75 (d, J=8.4 Hz, 2H), 3.00 (t, J=7.4 Hz, 2H), 2.77 (t, J=7.5 Hz, 2H), 2.43-2.36 (m, 4H), 2.19 (s, 3H), 2.10 (p, J=7.6 Hz, 2H), 1.76 (t, J=7.1 Hz, 2H).

### Example 300: 1-((5-((8-chloro-1,2,3,5,6,7-hexahydro-s-indacen-4-yl)amino)-1H-1,2,4-triazol-3-yl)sulfonyl)-N,N-dimethylpiperidin-4-amine

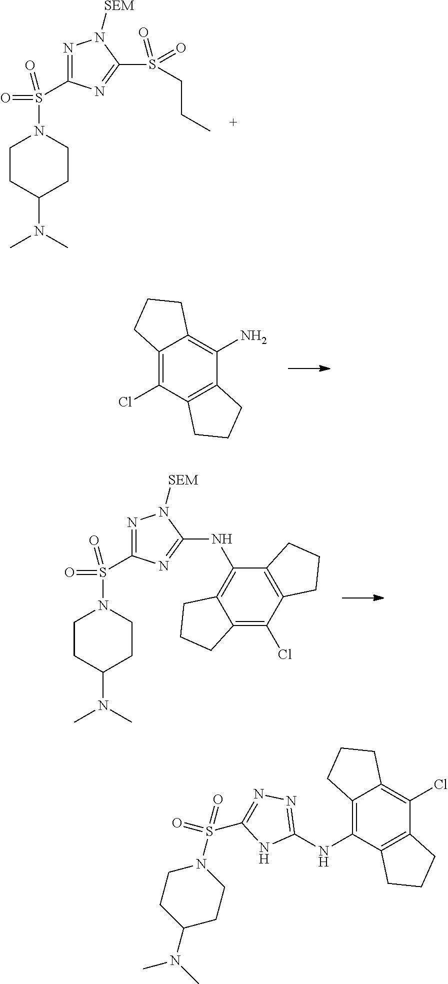 [SMILES: CCCS(=O)(=O)c1nc(S(=O)(=O)N2CCC(N(C)C)CC2)nn1COCC[Si](C)(C)C ; Nc1c2c(c(Cl)c3c1CCC3)CCC2 ; CN(C)C1CCN(S(=O)(=O)c2nc(Nc3c4c(c(Cl)c5c3CCC5)CCC4)n(COCC[Si](C)(C)C)n2)CC1 ; CN(C)C1CCN(S(=O)(=O)c2nnc(Nc3c4c(c(Cl)c5c3CCC5)CCC4)[nH]2)CC1]

LiHMDS (1 M in THF) (0.403 mL, 0.403 mmol) was added dropwise to N,N-dimethyl-1-((5-(propylsulfonyl)-1-((2-(trimethylsilyl)ethoxy)methyl)-1H-1,2,4-triazol-3-yl)sulfonyl)piperidin-4-amine (Intermediate D2) (100 mg, 0.202 mmol) and 8-chloro-1,2,3,5,6,7-hexahydro-s-indacen-4-amine (49 mg, 0.236 mmol) in THF (4 mL) at 0° C. The reaction stirred at RT for 3 h, quenched with sat aq NH₄Cl (15 mL) and extracted with EtOAc (2×20 mL). The organics were dried (phase separator) and concentrated in vacuo. The residue was dissolved in HCl (4 M in dioxane, 4 mL) and stirred at RT for 16 h. The crude was purified by acidic prep HPLC (20-40% MeOH in water) to afford the title compound (9 mg, 8%) as a white solid.

LCMS m/z 465.3/467.2 (33/35Cl M+H)+(ES⁺); 463.2/465.2 (33/35Cl M−H)⁻ (ES⁻).

¹H NMR (DMSO-d₆) δ 9.04 (s, 1H), 3.72-3.64 (m, 2H), 2.88 (t, J=7.5 Hz, 4H), 2.82-2.70 (m, 6H), 2.41-2.32 (m, 1H), 2.25 (s, 6H), 2.03 (p, J=7.4 Hz, 4H), 1.85-1.77 (m, 2H), 1.52-1.41 (m, 2H). One exchangeable proton not observed.

### Example 301: 1-((5-((2-cyclopropyl-4-fluoro-6-(2-methoxypyridin-4-yl)phenyl)amino)-1H-1,2,4-triazol-3-yl)sulfonyl)-N,N-dimethylpiperidin-4-amine

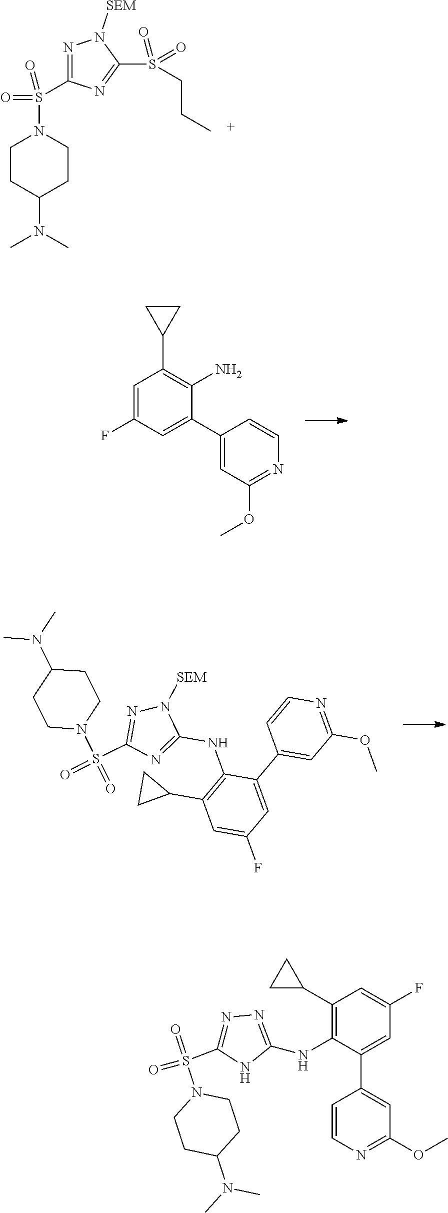

Prepared according to the general procedure of 1-((5-((8-chloro-1,2,3,5,6,7-hexahydro-s-indacen-4-yl)amino)-1H-1,2,4-triazol-3-yl)sulfonyl)-N,N-dimethylpiperidin-4-amine (Example 300) from N,N-dimethyl-1-((5-(propylsulfonyl)-1-((2-(trimethylsilyl)-ethoxy)methyl)-1H-1,2,4-triazol-3-yl)sulfonyl)piperidin-4-amine (Intermediate D2) and 2-cyclopropyl-4-fluoro-6-(2-methoxypyridin-4-yl)aniline (Intermediate C19) to afford the title compound (5 mg, 3%) as a pale tan solid.

LCMS m/z 516.3 (M+H)⁺ (ES⁺); 514.3 (M−H)⁻ (ES⁻).

¹H NMR (DMSO-d₆) δ 12.90 (br s, 1H), 8.96 (s, 1H), 8.15-8.11 (m, 1H), 7.09 (dd, J=8.9, 3.0 Hz, 1H), 6.97 (dd, J=5.2, 1.5 Hz, 1H), 6.91 (dd, J=10.0, 3.0 Hz, 1H), 6.79 (s, 1H), 3.83 (s, 3H), 3.65-3.56 (m, 2H), 2.71-2.56 (m, 2H), 2.47-2.33 (m, 7H), 2.05-1.98 (m. 1H), 1.95-1.84 (m, 2H), 1.56-1.44 (m, 2H), 0.92-0.86 (m, 2H), 0.76-0.69 (m, 2H).

### Example 302: 1-((5-((4-fluoro-2-isopropyl-6-(2-methylpyridin-4-yl)phenyl)amino)-1H-1,2,4-triazol-3-yl)sulfonyl)-N,N-dimethylpiperidin-4-amine

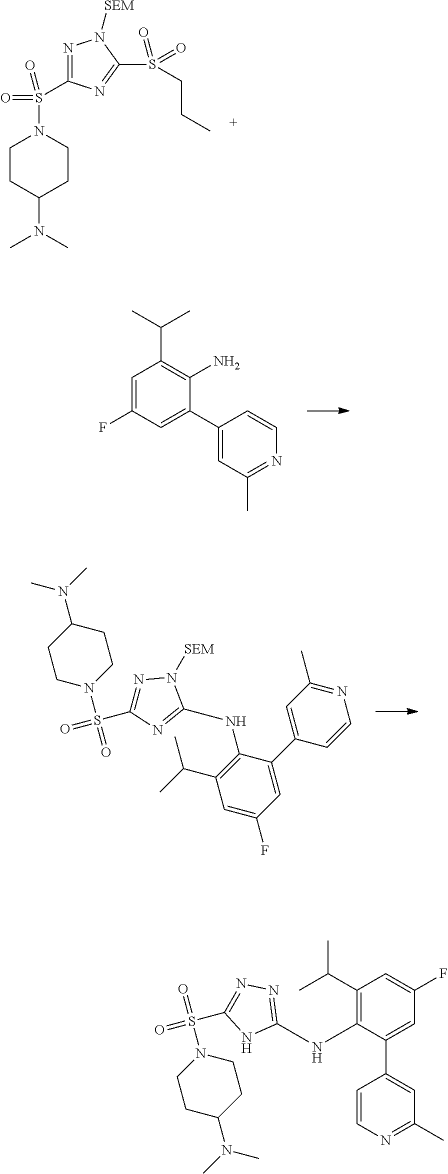 [SMILES: CCCS(=O)(=O)c1nc(S(=O)(=O)N2CCC(N(C)C)CC2)nn1COCC[Si](C)(C)C ; Cc1cc(-c2cc(F)cc(C(C)C)c2N)ccn1 ; Cc1cc(-c2cc(F)cc(C(C)C)c2Nc2nc(S(=O)(=O)N3CCC(N(C)C)CC3)nn2COCC[Si](C)(C)C)ccn1 ; Cc1cc(-c2cc(F)cc(C(C)C)c2Nc2nnc(S(=O)(=O)N3CCC(N(C)C)CC3)[nH]2)ccn1]

Prepared according to the general procedure of 1-((5-((8-chloro-1,2,3,5,6,7-hexahydro-s-indacen-4-yl)amino)-1H-1,2,4-triazol-3-yl)sulfonyl)-N,N-dimethylpiperidin-4-amine (Example 300) from N,N-dimethyl-1-((5-(propylsulfonyl)-1-((2-(trimethylsilyl)-ethoxy)methyl)-1H-1,2,4-triazol-3-yl)sulfonyl)piperidin-4-amine (Intermediate D2) and 4-fluoro-2-isopropyl-6-(2-methylpyridin-4-yl)aniline (Intermediate C9) to afford the title compound (25 mg, 22%) as a pale tan solid. LCMS m/z 502.4 (M+H)⁺ (ES⁺); 500.2 (M−H)⁻ (ES⁻).

¹H NMR (DMSO-d₆) δ 8.93 (s, 1H), 8.38 (s, 1H), 8.28 (d, J=4.9 Hz, 1H), 7.31 (dd, J=10.1, 3.0 Hz, 1H), 7.07 (d, J=5.0 Hz, 1H), 7.01 (dd, J=9.4, 3.0 Hz, 1H), 3.55-3.46 (m, 2H), 3.19 (septet, J=6.5 Hz, 1H), 2.46-233 (m, 2H), 2.31-2.18 (m, 7H), 2.09 (s, 3H), 1.83-1.72 (m, 2H), 1.45-134 (m, 2H), 1.17 (dd, J=18.7, 6.9 Hz, 6H). One exchangeable proton not observed.

### Example non: 1-((5-((4-fluoro-2,6-diisopropylphenyl)amino)-1H-1,2,4-triazol-3-yl)sulfonyl)-N,N-dimethylpiperidin-4-amine

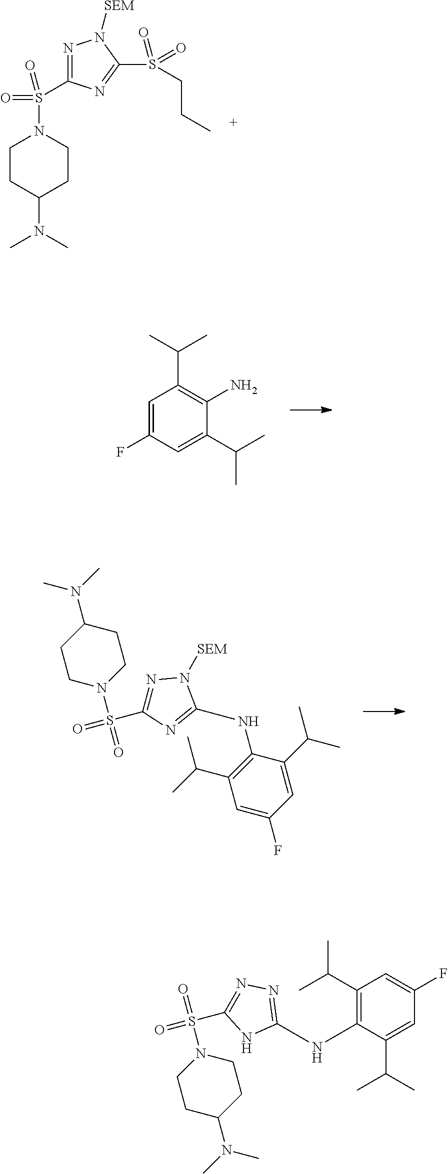 [SMILES: CCCS(=O)(=O)c1nc(S(=O)(=O)N2CCC(N(C)C)CC2)nn1COCC[Si](C)(C)C ; CC(C)c1cc(F)cc(C(C)C)c1N ; CC(C)c1cc(F)cc(C(C)C)c1Nc1nc(S(=O)(=O)N2CCC(N(C)C)CC2)nn1COCC[Si](C)(C)C ; CC(C)c1cc(F)cc(C(C)C)c1Nc1nnc(S(=O)(=O)N2CCC(N(C)C)CC2)[nH]1]

Prepared according to the general procedure of 1-((5-((8-chloro-1,2,3,5,6,7-hexahydro-s-indacen-4-yl)amino)-1H-1,2,4-triazol-3-yl)sulfonyl)-N,N-dimethylpiperidin-4-amine (Example 300) from N,N-dimethyl-1-((5-(propylsulfonyl)-1-((2-(trimethylsilyl)-ethoxy)methyl)-1H-1,2,4-triazol-3-yl)sulfonyl)piperidin-4-amine (Intermediate D2) and 4-fluoro-2,6-diisopropylaniline to afford the title compound (26 mg, 19%) as a white solid.

LCMS m/z 453.4 (M+H)⁺ (ES⁺); 451.3 (M−H)⁻ (ES⁻).

¹H NMR (DMSO-d₆) δ 12.87 (bs, 1H), 8.78 (s, 1H), 7.03 (d, J=9.9 Hz, 2H), 3.73-3.61 (m, 2H), 3.05 (septet, J=6.5 Hz, 2H), 2.79-2.68 (m, 2H), 2.41-2.11 (m, 7H), 1.83-1.73 (m. 2H), 1.50-1.37 (m 2H), 1.10 (d, J=6.8 Hz, 12H).

### Example 304: 5-((4-((dimethylamino)methyl)piperidin-1-yl)sulfonyl)-N-(1,2,3,5,6,7-hexahydro-s-indacen-4-yl)-4H-1,2,4-triazol-3-amine

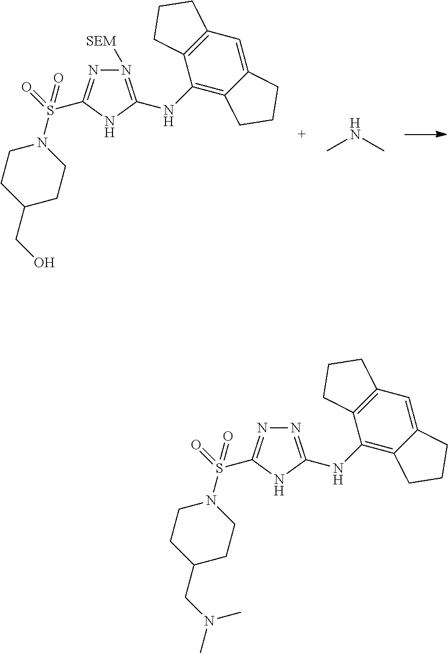

(1-((5-((1,2,3,5,6,7-hexahydro-s-indacen-4-yl)amino)-1-((2-(trimethylsilyl)ethoxy)-methyl)-1H-1,2,4-triazol-3-yl)sulfonyl)piperidin-4-yl)methanol (Intermediate D4) (0.166 g, 0.303 mmol) was dissolved in THF (2 mL) and DIPEA (0.074 mL, 0.424 mmol) was added. The solution was cooled to 0° C. and methanesulfonyl chloride (0.026 mL, 0.333 mmol) was added dropwise. The solution was warmed to RT and stirred for 2 h. Potassium iodide (5.00 mg, 0.030 mmol) was added, followed by dimethylamine (2 M in THF, 1.52 mL, 3.03 mmol) and the reaction was heated at 6° C. for 18 h. More dimethylamine (2 M in THF, 1.52 mL, 3.03 mmol) was added and the reaction was stirred at 6° C. for 24 h. More dimethylamine (2 M in THF, 3.03 mL, 6.06 mmol) was added and the reaction was stirred at 6° C. for a further 60 h. The reaction mixture was concentrated in vacuo and the residue was dissolved in HCl (4 M in dioxane, 4 mL) and stirred at RT for 16 h. The solution was concentrated in vacuo and the crude product was purified by reverse-phase chromatography on RP Flash C18 (12 g column, 0-75% MeCN/H₂O) to afford the title compound (11 mg, 8%) as a white solid.

LCMS m/z 445.3 (M+H)⁺ (ES⁺).

¹H NMR (DMSO-d₆) δ 8.93 (s, 1H), 6.96 (s, 1H), 3.71-3.60 (m, 2H), 2.82 (t, J=7.4 Hz, 4H), 2.70 (td, J=12.4, 2.6 Hz, 2H), 2.63 (t, J=7.4 Hz, 4H), 2.29 (br s, 8H), 1.97 (p, J=7.5 Hz, 4H), 1.79-1.72 (m, 2H), 1.60 (br s, 1H), 1.19-1.04 (m, 2H). One exchangeable proton not observed.

### Example 305: 5-((3-((dimethylamino)methyl)piperidin-1-yl)sulfonyl)-N-(1,2,3,5,6,7-hexahydro-s-indacen-4-yl)-4H-1,2,4-triazol-3-amine

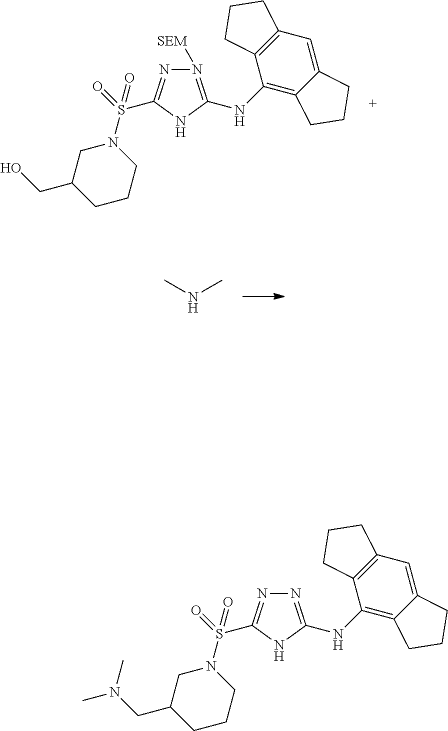

(1-((5-((1,2,3,5,6,7-hexahydro-s-indacen-4-yl)amino)-1-((2-(trimethylsilyl)ethoxy)-methyl)-1H-1,2,4-triazol-3-yl)sulfonyl)piperidin-3-yl)methanol(Intermediate D3) (0.187 g, 0.341 mmol) was dissolved in THF (2 mL) and DIPEA (0.083 mL, 0.477 mmol) was added. The solution was cooled to 0° C. and methanesulfonyl chloride (0.029 mL, 0.375 mmol) was added dropwise. The solution was warmed to RT and stirred for 3 h. More methanesulfonyl chloride (15 μL, 0.169 mmol) and DIPEA (41 μL, 0.237 mmol) were added and the reaction was stirred at RT for a further 2 h. Potassium iodide (6.00 mg, 0.036 mmol) was added, followed by dimethylamine (2 M in THF, 1.70 mL, 3.41 mmol) and the reaction was heated at 6° C. for 18 h. More dimethylamine (2 M in THF) (1.70 mL, 3.41 mmol) was added and the reaction was stirred at 6° C. for 24 h. The solution was concentrated in vacuo and the residue was redissolved in HCl (4M in dioxane, 4 mL, 16 mmol) and stirred at RT for 16 h. The solution was concentrated in vacuo and the crude product was purified by reverse-phase chromatography on RP Flash C18 (12 g column, 0-75% MeCN/H₂O to afford the title compound (8 mg, 5%) as a white solid.

LCMS m/z 445.3 (M+H)⁺ (ES⁺).

¹H NMR (DMSO-d₆) δ 9.04 (s, 1H), 6.95 (s, 1H), 3.81-3.67 (m, 1H), 3.60-3.52 (m, 1H), 2.82 (t, J=7.4 Hz, 4H), 2.73-2.61 (m, 5H), 2.44-2.34 (m, 1H), 2.30-2.15 (m, 8H), 1.97 (p, J=7.4 Hz, 4H), 1.83-1.72 (m, 1H), 1.72-1.62 (m, 2H), 1.57-1.42 (m, 1H).

Two exchangeable protons not observed.

### Example 306: (R)—N-(1,2,3,5,6,7-hexahydro-s-indacen-4-yl)-5-((3-(pyrrolidin-1-ylmethyl)pyrrolidin-1-yl)sulfonyl)-4H-1,2,4-triazol-3-amine

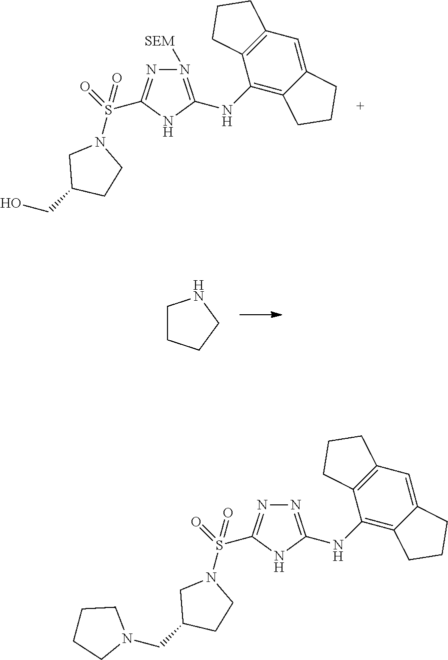

(S)-(1-((5-((1,2,3,5,6,7-hexahydro-s-indacen-4-yl)amino)-1-((2-(trimethylsilyl)ethoxy)-methyl)-1H-1,2,4-triazol-3-yl)sulfonyl)pyrrolidin-3-yl)methanol(IntermediateD5) (100 mg, 0.187 mmol) was dissolved in THF (1 mL) and DIPEA (58.8 μL, 0.338 mmol) was added. The solution was cooled to 0° C. and methanesulfonyl chloride (19.6 μL, 0.253 mmol) was added dropwise. The solution was warmed to RT and stirred for 2 h. Potassium iodide (4.00 mg, 0.024 mmol) was added, followed by pyrrolidine (198 μL, 2.41 mmol) and the reaction was heated at 6° C. for 18 h. The reaction mixture was concentrated in vacuo, the residue dissolved in HCl (4M in dioxane, 4 mL, 16 mmol) and stirred for 16 h at RT. The solution was concentrated in vacuo and the crude product was purified by acidic prep HPLC (20-50% MeOH in water) to afford the title compound (18 mg, 16%) as a pale yellow solid.

LCMS m/z 457.4 (M+H)⁺ (ES⁺).

¹H NMR (DMSO-d₆) δ 8.91 (s, 1H), 6.96 (s, 1H), 3.55-3.48 (m, 1H), 3.46-3.38 (m, 1H), 3.05-2.99 (m, 1H), 2.82 (t, J=7.4 Hz, 4H), 2.74-2.55 (m, 8H), 2.38-2.30 (m, 2H), 2.03-1.91 (m, 5H), 1.75 (s, 4H), 1.55-1.51 (m, 1H). Two exchangeable protons not observed. One aliphatic proton obscured by solvent.

### Example 307: (R)-5-((3-((cyclopropylamino)methyl)pyrrolidin-1-yl)sulfonyl)-N-(1,2,3,5,6,7-hexahydro-s-indacen-4-yl)-4H-1,2,4-triazol-3-amine

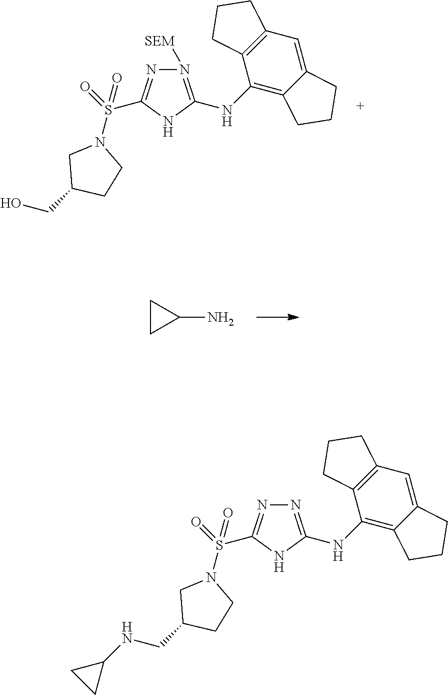

(S)-(1-((5-((1,2,3,5,6,7-hexahydro-s-indacen-4-yl)amino)-1-((2-(trimethylsilyl)ethoxy)-methyl)-1H-1,2,4-triazol-3-yl)sulfonyl)pyrrolidin-3-yl)methanol (Intermediate D5) (100 mg, 0.187 mmol) was dissolved in THF (1 mL) and DIPEA (58.8 μL, 0.338 mmol) was added. The solution was cooled to 0° C. and methanesulfonyl chloride (19.6 μL, 0.253 mmol) was added dropwise. The solution was warmed to RT and stirred for 2 h. Potassium iodide (4.00 mg, 0.024 mmol) was added, followed by cyclopropylamine (167 μL, 2.41 mmol) and the reaction was heated at 6° C. for 18 h. More cyclopropylamine (167 μL, 2.41 mmol) was added and the reaction was heated at 6° C. for 18 h. More cyclopropylamine (85 μL, 1.21 mmol) was added and the reaction was heated at 6° C. for 6 h. The reaction mixture was concentrated in vacuo, the residue dissolved in HCl in (4M in dioxane, 4 mL, 16 mmol) and stirred for 16 h at RT. The solution was concentrated in vacuo and the crude product was purified by acidic prep HPLC (20-50% MeOH in water) to afford the title compound (13 mg, 12%) as a pale yellow solid.

LCMS m/z 443.3 (M+H)⁺ (ES⁺).

¹H NMR (DMSO-d₆) δ 8.90 (s, 1H), 6.96 (s, 1H), 3.48-3.37 (m, 2H), 2.98 (dd, J=10.1, 7.5 Hz, 1H), 2.83 (t, J=7.4 Hz, 4H), 2.64 (t, J=7.3 Hz, 4H), 2.30-2.21 (in, 1H), 2.11 (s, 1H), 2.02-1.86 (m, 6H), 1.55-1.45 (m, 1H), 0.40 (d, J=6.8 Hz, 2H), 0.26 (s, 2H). Two exchangeable protons not observed. Two aliphatic protons obscured by solvent.

### Example 308: (R)-5-((3-((ethylamino)methyl)pyrrolidin-1-yl)sulfonyl)-N-(1,2,3,5,6,7-hexahydro-s-indacen-4-yl)-4H-1,2,4-triazol-3-amine

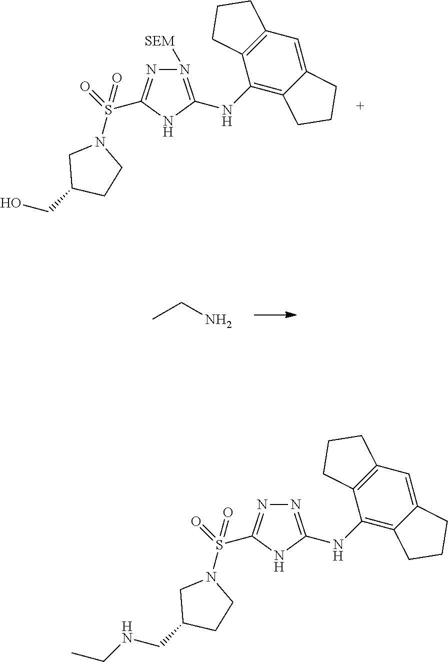

(S)-(1-((5-((1,2,3,5,6,7-hexahydro-s-indacen-4-yl)amino)-1-((2-(trimethylsilyl)ethoxy)-methyl)-1H-1,2,4-triazol-3-yl)sulfonyl)pyrrolidin-3-yl)methanol (Intermediate D5) (100 mg, 0.187 mmol) was dissolved in THF (1 mL) and DIPEA (58.8 μL, 0.338 mmol) was added. The solution was cooled to 0° C. and methanesulfonyl chloride (19.6 μL, 0.253 mmol) was added dropwise. The solution was warmed to RT and stirred for 2 h. Potassium iodide (4.00 mg, 0.024 mmol) was added, followed by ethylamine (2 M in THF, 1.21 mL, 2.41 mmol) and the reaction was heated at 6° C. for 18 h. More ethylamine (2 M in THF, 1.21 mL, 2.41 mmol) was added and the reaction was heated at 6° C. for 18 h. More ethylamine (2 M in THF, 603 μL, 1.21 mmol) was added and the reaction heated at 6° C. for 6 h. The reaction mixture was concentrated in vacuo, the residue dissolved HCl in (4M in dioxane, 4 mL, 16 mmol) and stirred for 16 h at RT. The solution was concentrated in vacuo and the crude product was purified by acidic prep HPLC (20-50% MeOH in water) to afford the title compound (17 mg, 16%) as a white solid.

LCMS m/z 431.3 (M+H)⁺ (ES⁺).

¹H NMR (DMSO-d₆): δ 9.12 (s, 1H), 6.94 (s, 1H), 3.54-3.46 (m, 1H), 3.45-3.38 (m, 1H), 3.35-3.28 (m, 2H), 3.11-2.98 (m, 1H), 2.82 (t, J=7.4 Hz, 4H), 2.78-2.58 (m, 7H), 2.36-2.30 (m, 2H), 2.05-1.88 (m, 5H), 1.63-1.50 (m, 1H), 1.14-1.01 (m, 3H). One exchangeable proton not observed.

### Example 309: (E)-1-(8-((5-((4-(dimethylamino)piperidin-1-yl)sulfonyl)-4H-1,2,4-triazol-3-yl)amino)-1,2,3,5,6,7-hexahydro-s-indacen-4-yl)ethan-1-one oxime and (Z)-1-(8-((5-((4-(dimethylamino)piperidin-1-yl)sulfonyl)-4H-1,2,4-triazol-3-yl)amino)-1,2,3,5,6,7-hexahydro-s-indacen-4-yl)ethan-1-one oxime

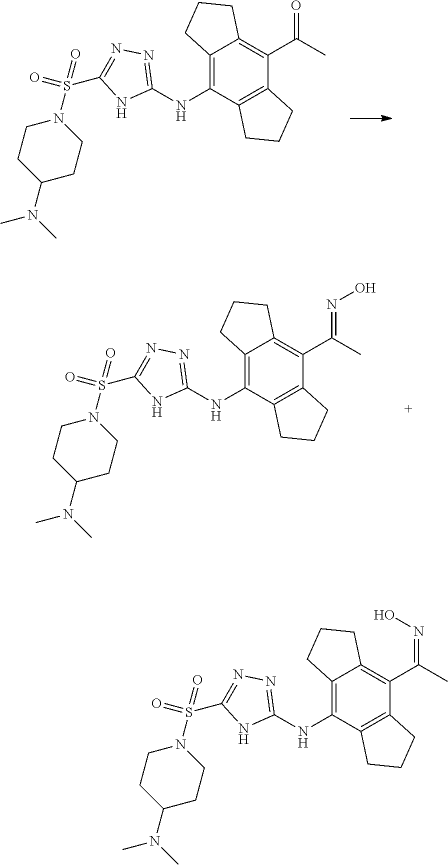

A mixture of 1-(8-((5-((4-(dimethylamino)piperidin-1-yl)sulfonyl)-4H-1,2,4-triazol-3-yl)amino)-1,2,3,5,6,7-hexahydro-s-indacen-4-yl)ethanone (Example 232) (20 mg, 0.042 mmol), hydroxylamine hydrochloride (5 mg, 0.072 mmol) and NaOAc (5 mg, 0.061 mmol) in MeOH (0.5 mL) was heated at 6° C. for 16 h. The volatiles were evaporated and the residue was dissolved in DMSO (1 mL), filtered and purified by acidic prep HPLC (10-40% MeOH in water) to afford a mixture of (E)-1-(8-((5-((4-(dimethylamino)piperidin-1-yl)sulfonyl)-4H-1,2,4-triazol-3-yl)amino)-1,2,3,5,6,7-hexahydro-s-indacen-4-yl)ethanone oxime and (Z)-1-(8-((5-((4-(dimethylamino)-piperidin-1-yl)sulfonyl)-4H-1,2,4-triazol-3-yl)amino)-1,2,3,5,6,7-hexahydro-s-indacen-4-yl)ethanone oxime (2 mg, 9%) as a white solid.

LCMS m/z 488.4 (M+H)⁺ (ES⁺).

¹H NMR (DMSO-d₆) δ 9.15 (s, 1H), 8.21 (s, 1H), 3.68-3.63 (m, 2H), 2.84-2.60 (m, 10H), 2.22-2.30 (m, 1H), 2.19 (s, 6H), 2.01 (s, 3H), 1.99-1.90 (m, 4H), 1.81-1.74 (m, 2H), 1.49-1.38 (m, 2H). One exchangeable proton not observed. Mixture of isomers.

### Example 310:2-((5-((5-(2-methoxypyridin-4-yl)-2,3-dihydro-1H-inden-4-yl)amino)-4H-1,2,4-triazol-3-yl)sulfonyl)benzonitrile

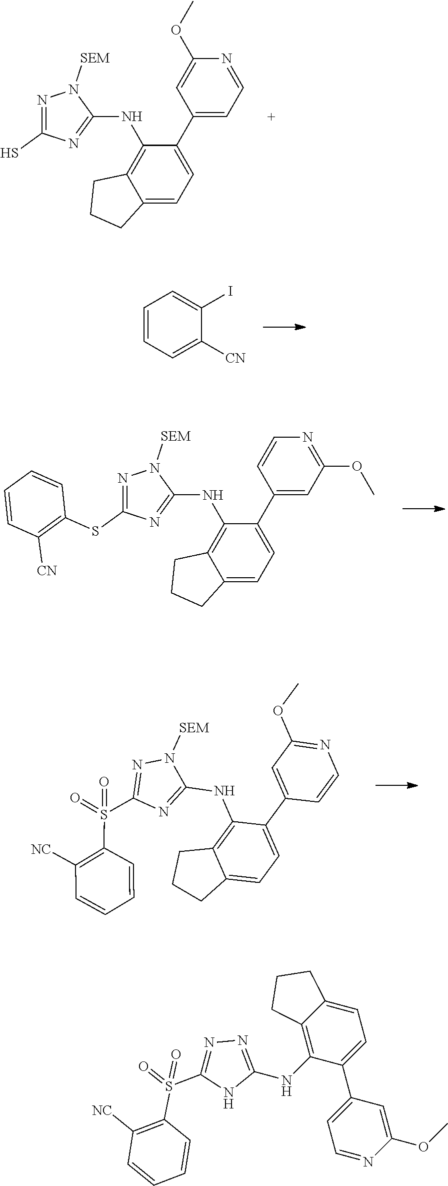 [SMILES: COc1cc(-c2ccc3c(c2Nc2nc(S)nn2COCC[Si](C)(C)C)CCC3)ccn1 ; N#Cc1ccccc1I ; COc1cc(-c2ccc3c(c2Nc2nc(Sc4ccccc4C#N)nn2COCC[Si](C)(C)C)CCC3)ccn1 ; COc1cc(-c2ccc3c(c2Nc2nc(S(=O)(=O)c4ccccc4C#N)nn2COCC[Si](C)(C)C)CCC3)ccn1 ; COc1cc(-c2ccc3c(c2Nc2nnc(S(=O)(=O)c4ccccc4C#N)[nH]2)CCC3)ccn1]

Step A: 2-Iodobenzonitrile (0.110 g, 0.479 mmol), XantPhos (0.021 g, 0.036 mmol), DIPEA (0.167 mL, 0.958 mmol) and Pd(dba)₂ (33 mg, 0.036 mmol) were added to a solution of 5-((5-(2-methoxypyridin-4-yl)-2,3-dihydro-1H-inden-4-yl)amino)-1-((2-(trimethylsilyl)ethoxy)methyl)-1H-1,2,4-triazole-3-thiol (Intermediate B54) (0.25 g, 0.479 mmol) in 1,4-dioxane (2 mL). The mixture was degassed with N₂, stirred at 100° C. for 18 h and concentrated to dryness. The product was purified by chromatography on silica gel (12 g column, 0-50% EtOAc/isohexane) to afford 2-((5-((5-(2-methoxy-pyridin-4-yl)-2,3-dihydro-1H-inden-4-yl)amino)-1-((2-(trimethylsilyl)ethoxy)methyl)-1H-1,2,4-triazol-3-yl)thio)benzonitrile (0.31 g, quant. yield) as a sticky brown foam.

LCMS m/z 571.3 (M+H)⁺ (ES⁺).

¹H NMR (DMSO-d₆) δ 8.73 (s, 1H), 8.10 (dd, J=5.3, 0.7 Hz, 1H), 7.87 (dd, J=7.7, 1.4 Hz, 1H), 7.62 (td, J=7.8, 1.5 Hz, 1H), 7.47 (td, J=7.7, 1.1 Hz, 1H), 7.27 (dd, J=8.1, 1.1 Hz, 1H), 7.23 (d, J=7.7 Hz, 1H), 7.17 (d, J=7.7 Hz, 1H), 6.92 (dd, J=5.3, 1.5 Hz, 1H), 6.77-6.71 (m, 1H), 5.31 (s, 2H), 3.84 (s, 3H), 3.54-3.45 (m, 2H), 2.92 (t, J=7.5 Hz, 2H), 2.66 (d, J=7.4 Hz, 2H), 2.04-1.92 (m, 2H), 0.88-0.74 (m, 2H), −0.05 (s, 9H).

Step B: m-CPBA (0.42 g, 1.874 mmol) was added to a solution of 2-((5-((5-(2-methoxypyridin-4-yl)-2,3-dihydro-1H-inden-4-yl)amino)-1-((2-(trimethylsilyl)ethoxy)-methyl)-1H-1,2,4-triazol-3-yl)thio)benzonitrile (0.31 g, 0.505 mmol) in DCM (10 mL) at 0° C. The mixture was stirred for 15 min and then warmed to RT and stirred for 18 h. More m-CPBA (200 mg) was added and the mixture was stirred for a further 6 h and quenched with aq sodium sulfite. The phases were separated and the aqueous phase was extracted with DCM (2×100 mL). The combined organic extracts were washed with sat aq NaHCO₃ (2×100 mL), dried (phase separator) and concentrated in vacuo. The crude was dissolved in MeCN/MeOH (30 mL, 2:1), B₂Pin₂ (0.13 g, 0.512 mmol) was added and the reaction was heated at 50° C. for 2 h. A further portion of B₂Pin₂ (0.13 g, 0.512 mmol) was added and the mixture was stirred at 50° C. for a further 1 h. After cooling, ethane-1,2-diamine (54 μL, 0.808 mmol) was added and the reaction was stirred for 1 h at RT, diluted with water (30 mL) and extracted with MTBE (3×40 mL). The combined organic phases were combined, dried (Na₂SO₄) and concentrated in vacuo to afford 2-((5-((5-(2-methoxypyridin-4-yl)-2,3-dihydro-1H-inden-4-yl)amino)-1-((2-(trimethylsilyl)ethoxy)methyl)-1H-1,2,4-triazol-3-yl)sulfonyl)benzonitrile (0.14 g, 25%) as a yellow residue.

LCMS m/z 603.3 (M+H)⁺ (ES⁺).

¹H NMR (DMSO-d₆) δ 9.06 (s, 1H), 8.21-8.09 (m, 2H), 8.07-7.95 (m, 5H), 7.25 (d, J=7.7 Hz, 1H), 7.16 (d, J=7.6 Hz, 1H), 6.79 (dd, J=5.3, 1.4 Hz, 1H), 6.67-6.64 (m, 1H), 5.39 (s, 2H), 3.80 (s, 3H), 3.50-3.42 (m, 2H), 2.91 (t, J=7.5 Hz, 2H), 1.92 (p, J=7.6 Hz, 2H), 0.82-0.73 (m, 2H), −0.07 (s, 9H).

Step C: 2-((5-((5-(2-Methoxypyridin-4-yl)-2,3-dihydro-1H-inden-4-yl)amino)-1-((2-(trimethylsilyl)ethoxy)methyl)-1H-1,2,4-triazol-3-yl)sulfonyl)benzonitrile (0.14 g, 0.128 mmol) was dissolved in TFA and stirred for 1 h. The mixture was concentrated in vacuo and the residue was dissolved in ethylenediamine (2 mL). The mixture was stirred for 1 h at RT, concentrated to −1 mL and purified by chromatography on RP Flash C18 (40 g column, 5-75% MeCN/water 0.1% formic acid) followed by chromatography on silica gel (12 g column, 0-100% EtOAc/isohexane) to afford the title compound (42 mg, 66%) as a white solid.

LCMS m/z 473.2 (M+H)⁺ (ES⁺); 471.1 (M−H)⁻ (ES⁻).

¹H NMR (DMSO-d₆) δ 13.25 (s, 1H), 9.07 (s, 1H), 8.18 (dd, J=7.1, 1.7 Hz, 1H), 8.14 (dd, J=7.6, 1.6 Hz, 1H), 8.03-7.95 (m, 3H), 7.24 (d, J=7.7 Hz, 1H), 7.16 (d, J=7.6 Hz, 1H), 6.80 (dd, J=5.3, 1.5 Hz, 1H), 6.65-6.60 (m, 1H), 3.80 (s, 3H), 2.92 (t, J=7.4 Hz, 2H), 2.58 (d, J=7.4 Hz, 2H), 1.97 (p, J=7.5 Hz, 2H).

### Example 311: 2-((5-((1,2,3,5,6,7-hexahydro-s-indacen-4-yl)amino)-1H-1,2,4-triazol-3-yl)sulfonyl)benzamide

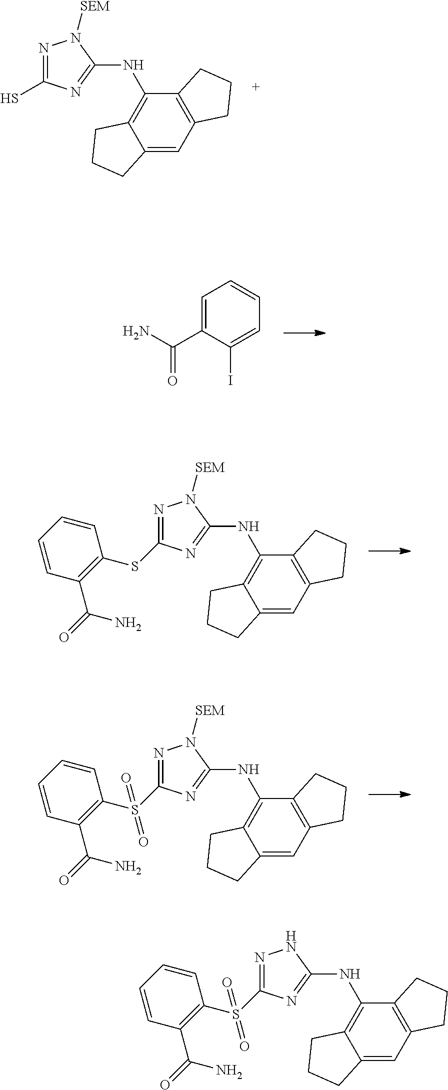 [SMILES: C[Si](C)(C)CCOCn1nc(S)nc1Nc1c2c(cc3c1CCC3)CCC2 ; NC(=O)c1ccccc1I ; C[Si](C)(C)CCOCn1nc(Sc2ccccc2C(N)=O)nc1Nc1c2c(cc3c1CCC3)CCC2 ; C[Si](C)(C)CCOCn1nc(S(=O)(=O)c2ccccc2C(N)=O)nc1Nc1c2c(cc3c1CCC3)CCC2 ; NC(=O)c1ccccc1S(=O)(=O)c1n[nH]c(Nc2c3c(cc4c2CCC4)CCC3)n1]

Step A: A solution of 5-((1,2,3,5,6,7-hexahydro-s-indacen-4-yl)amino)-1-((2-(trimethylsilyl)ethoxy)methyl)-1H-1,2,4-triazole-3-thiol (Intermediate B9) (200 mg, 0.497 mmol) in dioxane (2.4 mL) was added to 2-iodobenzamide (123 mg, 0.497 mmol), Pd(dba)₂ (34.1 mg, 0.037 mmol), XantPhos (21.56 mg, 0.037 mmol), and DIPEA (0.174 mL, 0.993 mmol). The mixture was stirred at 100° C. overnight and concentrated in vacuo. The crude was triturated using EtOAc (10 mL) and stirred for 30 min. The solid was filtered off and the filtrate was concentrated in vacuo to afford 2-((5-((1,2,3,5,6,7-hexahydro-s-indacen-4-yl)amino)-1-((2-(trimethylsilyl)ethoxy)-methyl)-1H-1,2,4-triazol-3-yl)thio)benzamide (291 mg, 90%) as a grey solid.

¹H NMR (DMSO-d₆) δ 8.53 (s, 1H), 7.94 (s, 1H), 7.56 (dd, J=7-5, 1.7 Hz, 1H), 7.47 (s, 1H), 7.31-7.18 (m, 2H), 7.05 (dd, J=7.8, 1.4 Hz, 1H), 6.91 (s, 1H), 5.46 (s, 2H), 3.67-3.61 (m, 2H), 2.78 (t, J=7.4 Hz, 4H), 2.63 (t, J=7.4 Hz, 4H), 1.93 (p, J=7.5 Hz, 4H), 0.94-0.86 (m, 2H), −0.00 (s, 9H).

Step B: 2-((5-((1,2,3,5,6,7-Hexahydro-s-indacen-4-yl)amino)-1-((2-(trimethylsilyl)-ethoxy)methyl)-1H-1,2,4-triazol-3-yl)thio)benzamide (291 mg, 0.558 mmol) was dissolved in DCM (20 mL) and cooled to 0° C. m-CPBA (241 mg, 1.394 mmol) was added and the reaction was stirred for 10 min at 0° C. then for 16 h at RT. The reaction was quenched with aq. sodium sulfite and the aqueous phase was extracted with DCM (2×5 mL). The combined organic extracts were washed with sat aq NaHCO₃ (2×50 mL), dried (phase separator) and concentrated in vacuo. The product was purified by chromatography on silica gel (4 g column, 0-10% MeOH/DCM) to afford 2-((5-((1,2,3,5,6,7-hexahydro-s-indacen-4-yl)amino)-1-((2-(trimethylsilyl)ethoxy)methyl)-1H-1,2,4-triazol-3-yl)sulfonyl)benzamide (50 mg, 11%) as a pale orange solid.

LCMS m/z 554.3 (M+H)⁺ (ES⁺).

Step C: HCl (4 M in dioxane, 3 mL, 12.00 mmol) was added to 2-((5-((1,2,3,5,6,7-hexahydro-s-indacen-4-yl)amino)-1-((2-(trimethylsilyl)ethoxy)methyl)-1H-1,2,4-triazol-3-yl)sulfonyl)benzamide (50 mg, 0.090 mmol) and stirred at RT for 16 h. The reaction was concentrated in vacuo and the product was purified by acidic prep HPLC (35-65% MeCN in water) to afford the title compound (1.8 mg, 5%) as a pale white solid.

LCMS m/z 424.2 (M+H)⁺ (ES⁺); 422.1 (M−H)⁻ (ES⁻).

¹H NMR (DMSO-d₆) δ 13.05, (br s, 2H), 8.78 (s, 1H), 7.97 (d, J=7.9 Hz, 1H), 7.79-7.74 (m, 1H), 7.69-7.64 (m, 1H), 7.53 (dd, J=7.6, 1.3 Hz, 1H), 7.49 (s, 1H), 6.91 (s, 1H), 2.79 (t, J=7.5 Hz, 4H), 1.91 (p, J=7.3 Hz, 4H). 4H masked by DMSO peak.

The compounds of examples 312-372 were synthesised by methods analogous to those outlined herein.

| Example | Structure | ¹H NMR spectrum | LCMS |
|---|---|---|---|
| 312 | 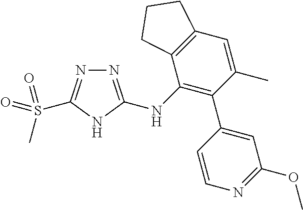 [SMILES: COc1cc(-c2c(C)cc3c(c2Nc2nnc(S(C)(=O)=O)[nH]2)CCC3)ccn1] | ¹H NMR (DMSO-d₆) δ 12.95 (br s, 1H), 8.60 (s, 1H), 8.13 (d, J = 5.2 Hz, 1H), 7.15 (s, 1H), 6.72 (dd, J = 5.2, 1.4 Hz, 1H), 6.56 (s, 1H), 3.84 (s, 3H), 3.17 (s, 3H), 2.92 (t, J = 7.4 Hz, 2H), 2.63 (t, J = 7.5 Hz, 2H), 2.06-1.95 (m, 5H). | m/z 400.2 (M + H)⁺ (ES+); 398.2 (M − H)⁻ (ES−) |
| | N-(5-(2-methoxypyridin-4-yl)-6-methyl- | | |
| | 2,3-dihydro-1H-inden-4-yl)-5- | | |
| | (methylsulfonyl)-4H-1,2,4-triazol-3- | | |
| | amine | | |
| 313 | 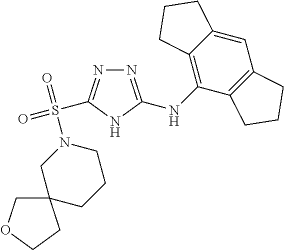 [SMILES: O=S(=O)(c1nnc(Nc2c3c(cc4c2CCC4)CCC3)[nH]1)N1CCCC2(CCOC2)C1] | ¹H NMR (methanol-d₄) δ 7.01 (s, 1H), 3.91-3.80 (m, 2H), 3.70 (d, J = 8.8 Hz, 1H), 3.44 (d, J = 8.8 Hz, 1H), 3.27-3.21 (m, 2H), 3.15 (d, J = 11.9 Hz, 1H), 3.10 (d, J = 11.9 Hz, 1H), 2.89 (t, J = 7.4 Hz, 4H), 2.73 (t, J = 7.3 Hz, 4H), 2.07 (p, J = 7.4 Hz, 4H), 1.94-1.85 (m, 1H), 1.78-1.52 (m, 5H). Two exchangable protons not observed. | m/z 444.3 (M + H)⁺ (ES+); 442.2 (M − H)⁻ (ES−) |
| | 5-(2-oxa-7-azaspiro[4.5]decan-7- | | |
| | ylsulfonyl)-N-(1,2,3,5,6,7-hexahydro-s- | | |
| | indacen-4-yl)-4H-1,2,4-triazol-3-amine | | |
| 314 | 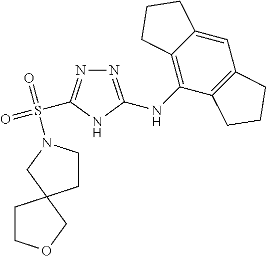 [SMILES: O=S(=O)(c1nnc(Nc2c3c(cc4c2CCC4)CCC3)[nH]1)N1CCC2(CCOC2)C1] | ¹H NMR (DMSO-d₆) δ 13.05 (br s, 1H), 8.95 (s, 1H), 6.97 (s, 1H), 3.75- 3.63 (m, 2H), 3.46-3.35 (m, 4H), 3.28 (s, 2H), 2.83 (t, J = 7.4 Hz, 4H), 2.63 (t, J = 7.3 Hz, 4H), 1.98 (p, J = 7.4 Hz, 4H), 1.86-1.76 (m, 2H), 1.69 (t, J = 7.1 Hz, 2H). | m/z 430.4 (M + H)⁺ (ES+) |
| | 5-(2-oxa-7-azaspiro[4.4]nonan-7- | | |
| | ylsulfonyl)-N-(1,2,3,5,6,7-hexahydro-s- | | |
| | indacen-4-yl)-4H-1,2,4-triazol-3-amine | | |
| 315 | 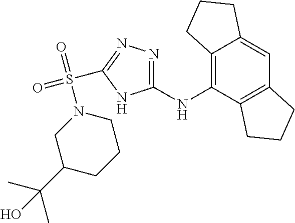 [SMILES: CC(C)(O)C1CCCN(S(=O)(=O)c2nnc(Nc3c4c(cc5c3CCC5)CCC4)[nH]2)C1] | ¹H NMR (DMSO-d₆) δ 13.05 (br s, 1H), 8.83 (s, 1H), 6.94 (s, 1H), 4.34 (s, 1H), 3.86 (d, J = 11.9 Hz, 1H), 3.63 (d, J = 11.8 Hz, 1H), 2.81 (t, J = 7.4 Hz, 4H), 2.63 (t, J = 8.0 Hz, 4H), 2.36 (t, J = 11.7 Hz, 1H), 1.96 (p, J = 7.1 Hz, 4H), 1.82-1.70 (m, 2H), 1.49-1.36 (m, 2H), 1.06 (s, 3H), 0.98 (s, 3H). 2H obscured by solvent. | m/z 446.2 (M + H)⁺ (ES+); 444.2 (M − H)⁻ (ES−) |
| | 2-(1-((5-((1,2,3,5,6,7-hexahydro-s- | | |
| | indacen-4-yl)amino)-4H-1,2,4-triazol-3- | | |
| | yl)sulfonyl)piperidin-3-yl)propan-2-ol | | |
| 316 | 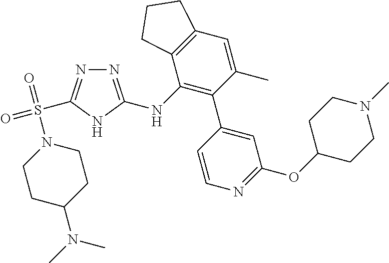 [SMILES: Cc1cc2c(c(Nc3nnc(S(=O)(=O)N4CCC(N(C)C)CC4)[nH]3)c1-c1ccnc(OC3CCN(C)CC3)c1)CCC2] | ¹H NMR (DMSO-d₆) δ 8.65 (s, 1H), 8.11 (d, J = 5.2 Hz, 1H), 7.16 (s, 1H), 6.71 (dd, J = 5.2, 1.4 Hz, 1H), 6.55 (s, 1H), 5.11- 5.04 (m, 1H), 3.71-3.63 (m, 2H), 3.11-3.01 (m, 2H), 2.93 (t, J = 7.5 Hz, 2H), 2.86-2.76 (m, 2H), 2.74-2.54 (m, 8H), 2.44 (s, 6H), 2.15-1.94 (m, 7H), 1.95-1.75 (m, 4H), 1.58- 1.47 (m, 2H). One exchangeable proton not observed. | m/z 595.4 (M + H)⁺ (ES+); 593.3 (M − H)⁻ (ES−) |
| | N,N-dimethyl-1-((5-((6-methyl-5-(2-((1- | | |
| | methylpiperidin-4-yl)oxy)pyridin-4-yl)- | | |
| | 2,3-dihydro-1H-inden-4-yl)amino)-4H- | | |
| | 1,2,4-triazol-3-yl)sulfonyl)piperidin-4- | | |
| | amine | | |
| 317 | 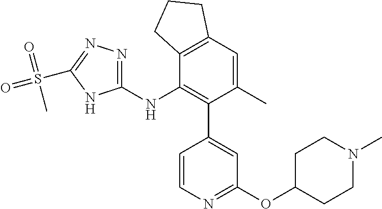 [SMILES: Cc1cc2c(c(Nc3nnc(S(C)(=O)=O)[nH]3)c1-c1ccnc(OC3CCN(C)CC3)c1)CCC2] | ¹H NMR (DMSO-d₆) δ 8.71 (s, 1H), 8.10 (d, J = 5.2 Hz, 1H), 7.15 (s, 1H), 6.69 (dd, J = 5.2, 1.4 Hz, 1H), 6.53-6.51 (m, 1H), 5.04-4.97 (m, 1H), 3.17 (s, 3H), 2.92 (t, J = 7.4 Hz, 2H), 2.86-2.78 (m, 2H), 2.64 (t, J = 7.5 Hz, 2H), 2.45-2.37 (m, 2H), 2.34 (s, 3H), 2.09-1.95 (m, 7H), 1.76-1.67 (m, 2H). One exchangeable proton not observed. | m/z 483.4 (M + H)⁺ (ES+); 481.2 (M − H)⁻ (ES−) |
| | N-(6-methyl-5-(2-((1-methylpiperidin- | | |
| | 4-yl)oxy)pyridin-4-yl)-2,3-dihydro-1H- | | |
| | inden-4-yl)-5-(methylsulfonyl)-4H- | | |
| | 1,2,4-triazol-3-amine | | |
| 318 | 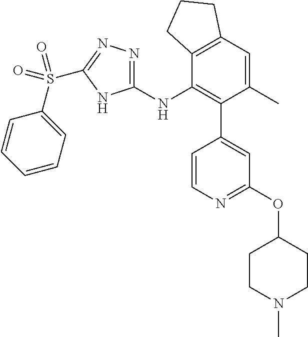 [SMILES: Cc1cc2c(c(Nc3nnc(S(=O)(=O)c4ccccc4)[nH]3)c1-c1ccnc(OC3CCN(C)CC3)c1)CCC2] | ¹H NMR (DMSO-d₆) δ 8.65 (s, 1H), 7.99 (d, J = 5.2 Hz, 1H), 7.89-7.84 (m, 2H), , 7.79-7.73 (m, 1H) 7.69-7.63 (m, 2H), 7.13 (s, 1H), 6.61 (dd, J = 5.2, 1.4 Hz, 1H), 6.47 (s, 1H), 5.03-4.96 (m, 1H), 2.95- 2.84 (m, 4H), 2.43 (s, 3H), 2.13-1.87 (m, 7H), 1.80-1.64 (m, 2H). 2 × CH₂ masked by DMSO peak. One exchangeable proton not observed. | m/z 545.3 (M + H)⁺ (ES+); 543.2 (M − H)⁻ (ES−) |
| | N-(6-methyl-5-(2-((1-methylpiperidin- | | |
| | 4-yl)oxy)pyridin-4-yl)-2,3-dihydro-1H- | | |
| | inden-4-yl)-5-(phenylsulfonyl)-4H- | | |
| | 1,2,4-triazol-3-amine | | |
| 319 | 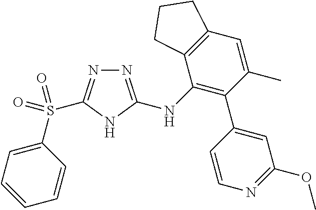 [SMILES: COc1cc(-c2c(C)cc3c(c2Nc2nnc(S(=O)(=O)c4ccccc4)[nH]2)CCC3)ccn1] | ¹H NMR (DMSO-d₆) δ 12.94 (br s, 1H), 8.53 (s, 1H), 8.00 (d, J = 5.2 Hz, 1H), 7.88-7.84 (m, 2H), 7.79-7.73 (m, 1H), 7.68- 7.63 (m, 2H), 7.12 (s, 1H), 6.61 (dd, J = 5.2, 1.4 Hz, 1H), 6.49 (s, 1H), 3.80 (s, 3H), 2.89 (t, J = 7.4 Hz, 2H), 2.48 (t, J = 7.4 Hz, 2H), 1.99 (s, 3H), 1.94 (p, J = 7.4 Hz, 2H). | m/z 462.3 (M + H)⁺ (ES+); 460.2 (M − H)⁻ (ES−) |
| | N-(5-(2-methoxypyridin-4-yl)-6-methyl- | | |
| | 2,3-dihydro-1H-inden-4-yl)-5- | | |
| | (phenylsulfonyl)-4H-1,2,4-triazol-3- | | |
| | amine | | |
| 320 | 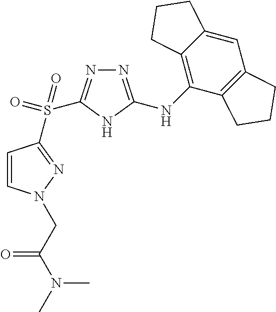 [SMILES: CN(C)C(=O)Cn1ccc(S(=O)(=O)c2nnc(Nc3c4c(cc5c3CCC5)CCC4)[nH]2)n1] | ¹H NMR (DMSO-d₆) δ 13.12 (br s, 1H), 8.98 (s, 1H), 7.88 (d, J = 2.5 Hz, 1H), 6.96 (s, 1H), 6.82 (d, J = 2.4 Hz, 1H), 5.28 (s, 2H), 3.02 (s, 3H), 2.86 (s, 3H), 2.82 (t, J = 7.5 Hz, 4H), 2.58 (t, J = 7.4 Hz, 4H), 1.96 (p, J = 7.4 Hz, 4H). | m/z 456.2 (M + H)⁺ (ES+); 454.1 (M − H)⁻ (ES−) |
| | 2-(3-((5-((1,2,3,5,6,7-hexahydro-s- | | |
| | indacen-4-yl)amino)-4H-1,2,4-triazol-3- | | |
| | yl)sulfonyl)-1H-pyrazol-1-yl)-N,N- | | |
| | dimethylacetamide | | |
| 321 | 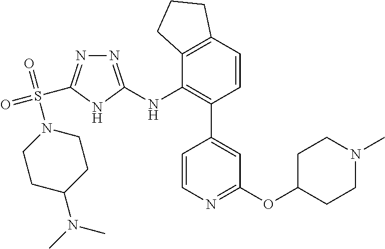 [SMILES: CN1CCC(Oc2cc(-c3ccc4c(c3Nc3nnc(S(=O)(=O)N5CCC(N(C)C)CC5)[nH]3)CCC4)ccn2)CC1] | ¹H NMR (DMSO-d₆) δ 13.15 (br s, 1H), 9.02 (s, 1H), 8.09 (d, J = 5.3 Hz, 1H), 7.27 (d, J = 7.7 Hz, 1H), 7.20 (d, J = 7.7 Hz, 1H), 6.93 (dd, J = 5.3, 1.2 Hz, 1H), 6.76 (s, 1H), 5.23-5.13 (m, 1H), 3.79- 3.71 (m, 2H), 2.96 (t, J = 7.5 Hz, 2H), 2.87-2.67 (m, 10H), 2.62 (t, J = 7.3 Hz, 2H), 2.32-1.71 (m, 8H), 1.69-1.58 (m, 2H). 6H masked by water peak. | m/z 581.4 (M + H)⁺ (ES+); 579.4 (M − H)⁻ (ES−) |
| | N,N-dimethyl-1-((5-((5-(2-((1- | | |
| | methylpiperidin-4-yl)oxy)pyridin-4-yl)- | | |
| | 2,3-dihydro-1H-inden-4-yl)amino)-4H- | | |
| | 1,2,4-triazol-3-yl)sulfonyl)piperidin-4- | | |
| | amine | | |
| 322 | 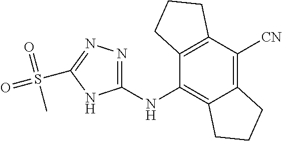 [SMILES: CS(=O)(=O)c1nnc(Nc2c3c(c(C#N)c4c2CCC4)CCC3)[nH]1] | ¹H NMR (DMSO-d₆) δ 13.58 (br s, 1H), 9.38 (s, 1H), 3.25 (s, 3H), 2.98 (t, J = 7.5 Hz, 4H), 2.67 (t, J = 7.4 Hz, 4H), 1.98 (p, J = 7.4 Hz, 4H). | m/z 3 44.1 (M + H)⁺ (ES+); 342.1 (M − H)⁻ (ES−) |
| | 8-((5-(methylsulfonyl)-4H-1,2,4-triazol- | | |
| | 3-yl)amino)-1,2,3,5,6,7-hexahydro-s- | | |
| | indacene-4-carbonitrile | | |
| 323 | 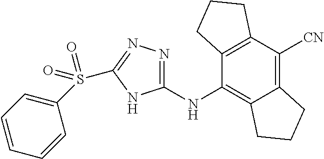 [SMILES: N#Cc1c2c(c(Nc3nnc(S(=O)(=O)c4ccccc4)[nH]3)c3c1CCC3)CCC2] | ¹H NMR (DMSO-d₆) δ 13.65 (br s, 1H), 9.32 (s, 1H), 7.96-7.91 (m, 2H), 7.80-7.75 (m, 1H),. 7.70- 7.64 (m, 2H), 2.94 (t, J = 7.5 Hz, 4H), 1.98 (p, J = 7.4 Hz, 4H). 2 × CH₂ masked by DMSO peak. | m/z 406.23 (M + H)⁺ (ES+); 404.2 (M − H)⁻ (ES−) |
| | 8-((5-(phenylsulfonyl)-4H-1,2,4-triazol- | | |
| | 3-yl)amino)-1,2,3,5,6,7-hexahydro-5- | | |
| | indacene-4-carbonitrile | | |
| 324 | 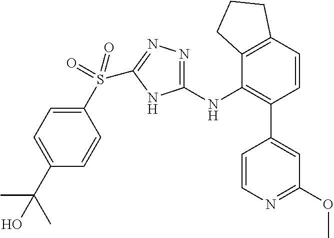 [SMILES: COc1cc(-c2ccc3c(c2Nc2nnc(S(=O)(=O)c4ccc(C(C)(C)O)cc4)[nH]2)CCC3)ccn1] | ¹H NMR (DMSO-d₆) δ 13.09 (br s, 1H), 8.98 (s, 1H), 8.01 (dd, J = 5.3, 0.7 Hz, 1H), 7.81-7.76 (m, 2H), 7.76-7.69 (m, 2H), 7.25 (d, J = 7.7 Hz, 1H), 7.17 (d, J = 7.6 Hz, 1H), 6.83 (dd, J = 5.3, 1.5 Hz, 1H), 6.68-6.66 (m, 1H), 5.29 (s, 1H), 3.80 (s, 3H), 2.93 (t, J = 7.4 Hz, 2H), 2.56 (t, J = 7.4 Hz, 2H), 1.97 (p, J = 7.5 Hz, 2H), 1.45 (s, 6H). | m/z 506.3 (M + H)⁺ (ES+); 504.2 (M − H)⁻ (ES−) |
| | 2-(4-((5-((5-(2-methoxypyridin-4-yl)- | | |
| | 2,3-dihydro-1H-inden-4-yl)amino)-4H- | | |
| | 1,2,4-triazol-3- | | |
| | yl)sulfonyl)phenyl)propan-2-ol | | |
| 325 | 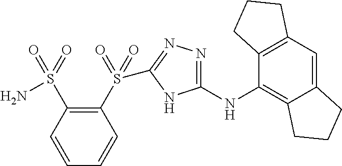 [SMILES: NS(=O)(=O)c1ccccc1S(=O)(=O)c1nnc(Nc2c3c(cc4c2CCC4)CCC3)[nH]1] | ¹H NMR (DMSO-d₆) δ 8.82 (br s, 1H), 8.23 (d, J = 7.8 Hz, 1H), 8.17 (dd, J = 7.8, 1.5 Hz, 1H), 7.97 (dd, J = 8.2, 6.6 Hz, 1H), 7.94-7.89 (m, 1H), 7.22 (br s, 2H), 6.91 (s, 1H), 2.78 (t, J = 7.4 Hz, 4H), 2.55 (t, J = 7.4 Hz, 4H), 1.92 (p, J = 7.4 Hz, 4H). One exchangeable proton | m/z 460.2 (M + H)⁺ (ES+); 458.1 (M − H)⁻ (ES−) |
| | 2-((5-((1,2,3,5,6,7-hexahydro-s-indacen- | not observed. | |
| | 4-yl)amino)-4H-1,2,4-triazol-3- | | |
| | yl)sulfonyl)benzenesulfonamide | | |
| 326 | 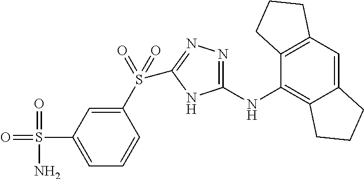 [SMILES: NS(=O)(=O)c1cccc(S(=O)(=O)c2nnc(Nc3c4c(cc5c3CCC5)CCC4)[nH]2)c1] | ¹H NMR (DMSO-d₆) δ 9.11 (s, 1H), 8.34 (t, J = 1.9 Hz, 1H), 8.19-8.10 (m, 2H), 7.93-7.85 (m, 1H), 6.93 (s, 1H), 2.80 (t, J = 7.3 Hz, 4H), 1.92 (p, J = 7.4 Hz, 4H). 4H masked by DMSO peak, 3 exchangeable protons not observed. | m/z 460.2 (M + H)⁺ (ES+); 458.1 (M − H)⁻ (ES−) |
| | 3-((5-((1,2,3,5,6,7-hexahydro-s-indacen- | | |
| | 4-yl)amino)-4H-1,2,4-triazol-3- | | |
| | yl)sulfonyl)benzenesulfonamide | | |
| 327 | 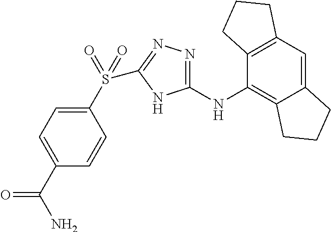 [SMILES: NC(=O)c1ccc(S(=O)(=O)c2nnc(Nc3c4c(cc5c3CCC5)CCC4)[nH]2)cc1] | ¹H NMR (DMSO-d₆) δ 8.99 (s, 1H), 8.21 (s, 1H), 8.11-8.06 (m, 2H), 8.01- 7.97 (m, 2H), 7.66 (s, 1H), 6.93 (s, 1H), 2.79 (t, J = 7.4 Hz, 4H), 1.91 (p, J = 7.5 Hz, 4H). One exchangeable not observed and 2 × CH₂ protons masked by DMSO peak. | m/z 424.2 (M + H)⁺ (ES+); 422.1 (M − H)⁻ (ES−) |
| | 4-((5-((1,2,3,5,6,7-hexahydro-s-indacen- | | |
| | 4-yl)amino)-4H-1,2,4-triazol-3- | | |
| | yl)sulfonyl)benzamide | | |
| 328 | 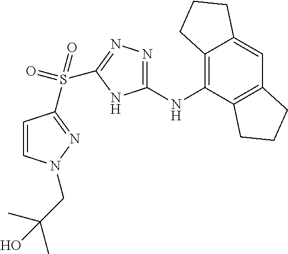 [SMILES: CC(C)(O)Cn1ccc(S(=O)(=O)c2nnc(Nc3c4c(cc5c3CCC5)CCC4)[nH]2)n1] | ¹H NMR (DMSO-d₆) δ 8.97 (s, 1H), 7.87 (d, J = 2.4 Hz, 1H), 6.94 (s, 1H), 6.81 (d, J = 2.4 Hz, 1H), 4.80 (s, 1H), 4.12 (s, 2H), 2.81 (t, J = 7.4 Hz, 4H), 2.60-2.53 (m, 4H), 1.94 (p, J = 7.4 Hz, 4H), 1.03 (s, 6H). One exchangeable proton not observed. | m/z 443.2 (M + H)⁺ (ES+); 441.2 (M − H)⁻ (ES−) |
| | 1-(3-((5-((1,2,3,5,6,7-hexahydro-s- | | |
| | indacen-4-yl)amino)-4H-1,2,4-triazol-3- | | |
| | yl)sulfonyl)-1H-pyrazol-1-yl)-2- | | |
| | methylpropan-2-ol | | |
| 329 | 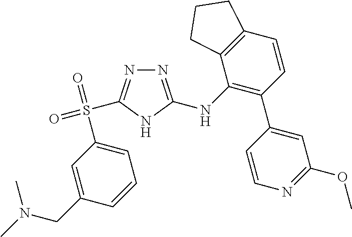 | ¹H NMR (DMSO-d₆) δ 13.10 (br s, 1H), 8.95 (br s, 1H), 8.00 (d, J = 5.3 Hz, 1H), 7.83-7.77 (m, 1H), 7.76-7.70 (m, 1H), 7.67- 7.63 (m, 1H), 7.60 (t, J = 7.6 Hz, 1H), 7.25 (d, J = 7.7 Hz, 1H), 7.17 (d, J = 7.7 Hz, 1H), 6.82 (dd, J = 5.2, 1.4 Hz, 1H), 6.68- 6.64 (m, 1H), 3.79 (s, 3H), 3.50 (s, 2H), 2.92 (t, J = 7.5 Hz, 2H), 2.59-2.41 (m, assumed 2H, obscured by solvent)), | m/z 505.3 (M + H)⁺ (ES+); 503.2 (M − H)⁻ (ES−) |
| | 5-((3-((dimethylamino)methyl)- | 2.14 (s, 6H), 1.96 (p, J = | |
| | phenyl)sulfonyl)-N-(5-(2- | 7.5 Hz, 2H). | |
| | methoxypyridin-4-yl)-2,3-dihydro-1H- | | |
| | inden-4-yl)-4H-1,2,4-triazol-3-amine | | |
| 330 | 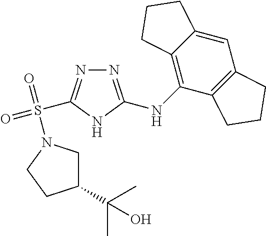 [SMILES: CC(C)(O)[C@@H]1CCN(S(=O)(=O)c2nnc(Nc3c4c(cc5c3CCC5)CCC4)[nH]2)C1] | ¹H NMR (DMSO-d₆) δ 13.02 (s, 1H), 8.89 (s, 1H), 6.95 (s, 1H), 4.35 (br s, 1H), 3.46-3.41 (m, 1H), 3.37-3.32 (m, 1H), 3.28- 3.22 (m, 1H), 3.10 (t, J = 9.9 Hz, 1H), 2.82 (t, J = 7.5 Hz, 4H), 2.63 (t, J = 7.4 Hz, 4H), 2.10-2.01 (m, 1H), 1.97 (p, J = 7.4 Hz, 4H), 1.80-1.74 (m, 1H), 1.72-1.62 (m, 1H), 1.01 (s, 6H). | m/z 432.4 (M + H)⁺ (ES+); 430.3 (M − H)⁻ (ES−) |
| | (R)-2-(1-((5-((1,2,3,5,6,7-hexahydro-s- | | |
| | indacen-4-yl)amino)-4H-1,2,4-triazol-3- | | |
| | yl)sulfonyl)pyrrolidin-3-yl)propan-2-ol | | |
| 331 | 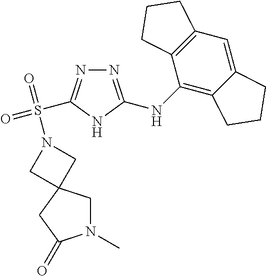 [SMILES: CN1CC2(CC1=O)CN(S(=O)(=O)c1nnc(Nc3c4c(cc5c3CCC5)CCC4)[nH]1)C2] | ¹H NMR (DMSO-d₆) δ 13.23 (s, 1H), 9.03 (s, 1H), 6.98 (s, 1H), 3.97-3.91 (m, 4H), 3.35 (s, 2H), 2.84 (t, J = 7.4 Hz, 4H), 2.67 (t, J = 7.5 Hz, 4H), 2.64 (s, 3H), 2.38 (s, 2H), 1.99 (p, J = 7.5 Hz, 4H). | m/z 443.2 (M + H)⁺ (ES+); 441.3 (M − H)⁻ (ES−) |
| | 2-((5-((1,2,3,5,6,7-hexahydro-s-indacen- | | |
| | 4-yl)amino)-4H-1,2,4-triazol-3- | | |
| | yl)sulfonyl)-6-methyl-2,6- | | |
| | diazaspiro[3.4]octan-7-one | | |
| 332 | 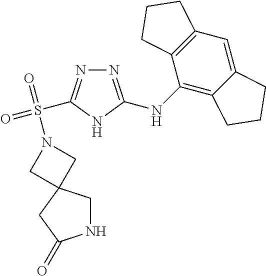 [SMILES: O=C1CC2(CN1)CN(S(=O)(=O)c1nnc(Nc3c4c(cc5c3CCC5)CCC4)[nH]1)C2] | ¹H NMR (DMSO-d₆) δ 13.23 (s, 1H), 9.03 (s, 1H), 7.62 (s, 1H), 6.98 (s, 1H), 3.97-3.87 (m, 4H), 3.26 (s, 2H), 2.84 (t, J = 7.4 Hz, 4H), 2.66 (t, J = 7.3 Hz, 4H), 2.26 (s, 2H), 1.99 (p, J = 7.4 Hz, 4H). | m/z 428.9 (M + H)⁺ (ES+); 427.3 (M − H)⁻ (ES−) |
| | 2-((5-((1,2,3,5,6,7-hexahydro-s-indacen- | | |
| | 4-yl)amino)-4H-1,2,4-triazol-3- | | |
| | yl)sulfonyl)-2,6-diazaspiro[3.4]octan-7- | | |
| | one | | |
| 333 | 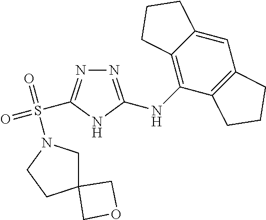 [SMILES: O=S(=O)(c1nnc(Nc2c3c(cc4c2CCC4)CCC3)[nH]1)N1CCC2(COC2)C1] | ¹H NMR (DMSO-d₆) δ 13.02 (br s, 1H), 8.93 (s, 1H), 6.97 (s, 1H), 4.36 (d, J = 6.1 Hz, 2H), 4.32 (d, J = 6.1 Hz, 2H), 3.54 (s, 2H), 2.82 (t, J = 7.4 Hz, 4H), 2.62 (t, J = 7.3 Hz, 4H), 2.09 (t, J = 6.9 Hz, 2H), 1.97 (p, J = 7.4 Hz, 4H). 1 × CH₂ masked by water peak. | m/z 416.3 (M + H)⁺ (ES+) |
| | 5-(2-oxa-6-azaspiro[3.4]octan-6- | | |
| | ylsulfonyl)-N-(1,2,3,5,6,7-hexahydro-s- | | |
| | indacen-4-yl)-4H-1,2,4-triazol-3-amine | | |
| 334 | 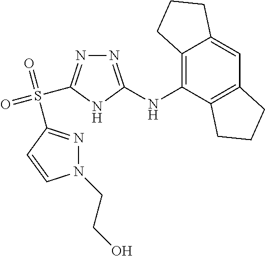 [SMILES: O=S(=O)(c1ccn(CCO)n1)c1nnc(Nc2c3c(cc4c2CCC4)CCC3)[nH]1] | ¹H NMR (DMSO-d₆) δ 8.99 (s, 1H), 7.95 (d, J = 2.3 Hz, 1H), 6.94 (s, 1H), 6.79 (d, J = 2.4 Hz, 1H), 5.00 (br s, 1H), 4.25 (t, J = 5.4 Hz, 2H), 3.74 (t, J = 5.5 Hz, 2H), 2.81 (t, J = 7.4 Hz, 4H), 2.57 (t, J = 7.3 Hz, 4H), 1.95 (p, J = 7.4 Hz, 4H). One exchangeable proton not observed. | m/z 415.2 (M + H)⁺ (ES+) |
| | 2-(3-((5-((1,2,3,5,6,7-hexahydro-s- | | |
| | indacen-4-yl)amino)-4H-1,2,4-triazol-3- | | |
| | yl)sulfonyl)-1H-pyrazol-1-yl)ethanol | | |
| 335 | 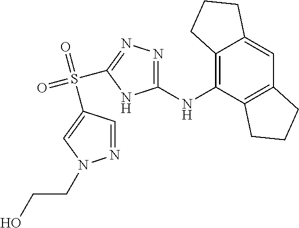 [SMILES: O=S(=O)(c1cnn(CCO)c1)c1nnc(Nc2c3c(cc4c2CCC4)CCC3)[nH]1] | ¹H NMR (DMSO-d₆) δ 9.02 (s, 1H), 8.42 (s, 1H), 7.88 (s, 1H), 6.94 (s, 1H), 4.97 (br s, 1H), 4.21 (t, J = 5.5 Hz, 2H), 3.75 (t, J = 5.4 Hz, 2H), 2.81 (t, J = 7.4 Hz, 4H), 2.57 (t, J = 7.3 Hz, 4H), 1.94 (p, J = 7.5 Hz, 4H). One exchangeable proton not observed. | m/z 415.2 (M + H)⁺ (ES+) |
| | 2-(4-((5-((1,2,3,5,6,7-hexahydro-s- | | |
| | indacen-4-yl)amino)-4H-1,2,4-triazol-3- | | |
| | yl)sulfonyl)-1H-pyrazol-1-yl)ethanol | | |
| 336 | 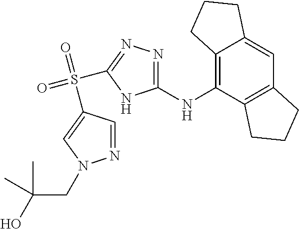 [SMILES: CC(C)(O)Cn1cc(S(=O)(=O)c2nnc(Nc3c4c(cc5c3CCC5)CCC4)[nH]2)cn1] | ¹H NMR (DMSO-d₆) δ 9.02 (s, 1H), 8.29 (s, 1H), 7.86 (s, 1H), 6.93 (s, 1H), 4.79 (br s, 1H), 4.09 (s, 2H), 2.80 (t, J = 7.4 Hz, 4H), 2.56 (t, J = 7.3 Hz, 4H), 1.94 (p, J = 7.5 Hz, 4H), 1.06 (s, 6H). One exchangeable proton not observed. | m/z 443.3 (M + H)⁺ (ES+) |
| | 1-(4-((5-((1,2,3,5,6,7-hexahydro-s- | | |
| | indacen-4-yl)amino)-4H-1,2,4-triazol-3- | | |
| | yl)sulfonyl)-1H-pyrazol-1-yl)-2- | | |
| | methylpropan-2-ol | | |
| 337 | 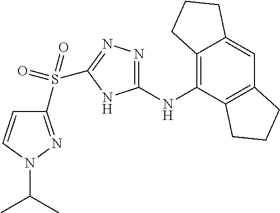 [SMILES: CC(C)n1ccc(S(=O)(=O)c2nnc(Nc3c4c(cc5c3CCC5)CCC4)[nH]2)n1] | ¹H NMR (DMSO-d₆) δ 8.95 (s, 1H), 8.04 (d, J = 2.5 Hz, 1H), 6.93 (s, 1H), 6.79 (d, J = 2.4 Hz, 1H), 4.61 (sept, J = 6.7 Hz, 1H), 2.80 (t, J = 7.4 Hz, 4H), 2.55 (t, J = 7.3 Hz, 4H), 1.93 (p, J = 7.4 Hz, 4H), 1.42 (d, J = 6.7 Hz, 6H). One exchangeable proton not observed. | m/z 413.2 (M + H)⁺ (ES+) |
| | N-(1,2,3,5,6,7-hexahydro-s-indacen-4- | | |
| | yl)-5-((1-isopropyl-1H-pyrazol-3- | | |
| | yl)sulfonyl)-4H-1,2,4-triazol-3-amine | | |
| 338 | 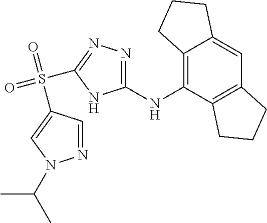 [SMILES: CC(C)n1cc(S(=O)(=O)c2nnc(Nc3c4c(cc5c3CCC5)CCC4)[nH]2)cn1] | ¹H NMR (DMSO-d₆) δ 8.96 (s, 1H), 8.53 (s, 1H), 7.88 (s, 1H), 6.93 (s, 1H), 4.59 (sept, J = 6.6 Hz, 1H), 2.80 (t, J = 7.4 Hz, 4H), 2.56 (t, J = 7.3 Hz, 4H), 1.94 (p, J = 7.4 Hz, 4H), 1.42 (d, J = 6.7 Hz, 6H). One exchangeable proton not observed. | m/z 413.3 (M + H)⁺ (ES+) |
| | N-(1,2,3,5,6,7-hexahydro-s-indacen-4- | | |
| | yl)-5-((1-isopropyl-1H-pyrazol-4- | | |
| | yl)sulfonyl)-4H-1,2,4-triazol-3-amine | | |
| 339 | 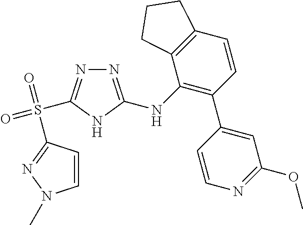 [SMILES: COc1cc(-c2ccc3c(c2Nc2nnc(S(=O)(=O)c4ccn(C)n4)[nH]2)CCC3)ccn1] | ¹H NMR (DMSO-d₆) δ 13.05 (br s, 1H), 8.93 (br s, 1H), 8.05 (d, J = 5.3 Hz, 1H), 7.94 (d, J = 2.4 Hz, 1H), 7.25 (d, J = 7.7 Hz, 1H), 7.17 (d, J = 7.7 Hz, 1H), 6.86 (dd, J = 5.3, 1.5 Hz, 1H), 6.73 (d, J = 2.4 Hz, 1H), 6.69-6.67 (m, 1H), 3.94 (s, 3H), 3.81 (s, 3H), 2.93 (t, J = 7.4 Hz, 2H), 2.59 (t, J = 7.4 Hz, 2H), 1.99 (p, J = 7.4 Hz, 2H). | m/z 452.4 (M + H)⁺ (ES+); 450.2 (M − H)⁻ (ES−) |
| | N-(5-(2-methoxypyridin-4-yl)-2,3- | | |
| | dihydro-1H-inden-4-yl)-5-((1-methyl- | | |
| | 1H-pyrazol-3-yl)sulfonyl)-4H-1,2,4- | | |
| | triazol-3-amine | | |
| 340 | 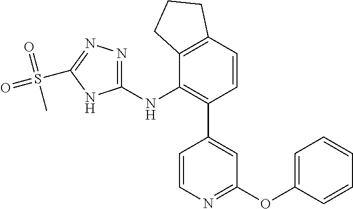 [SMILES: CS(=O)(=O)c1nnc(Nc2c(-c3ccnc(Oc4ccccc4)c3)ccc3c2CCC3)[nH]1] | ¹H NMR (DMSO-d₆) δ 13.15 (br s, 1H), 9.05 (s, 1H), 8.10 (dd, J = 5.3, 1.5 Hz, 1H), 7.42-7.37 (m, 2H), 7.27 (d, J = 7.7 Hz, 1H), 7.25 (d, J = 7.7 Hz, 1H), 7.21-7.17 (m, 1H), 7.11-7.05 (m, 3H), 6.93- 6.90 (m, 1H), 3.16 (s, 3H), 2.96 (t, J = 7.4 Hz, 2H), 2.72 (t, J = 7.4 Hz, 2H), 2.04 (p, J = 7.4 Hz, 2H). | m/z 448.3 (M + H)⁺ (ES+); 446.2 (M − H)⁻ (ES−) |
| | 5-(methylsulfonyl)-N-(5-(2- | | |
| | phenoxypyridin-4-yl)-2,3-dihydro-1H- | | |
| | inden-4-yl)-4H-1,2,4-triazol-3-amine | | |
| 341 | 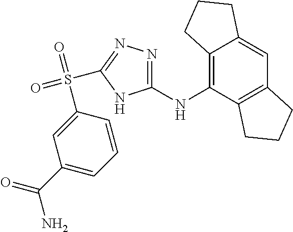 [SMILES: NC(=O)c1cccc(S(=O)(=O)c2nnc(Nc3c4c(cc5c3CCC5)CCC4)[nH]2)c1] | ¹H NMR (DMSO-d₆) δ 8.99 (s, 1H), 8.41 (t, J = 1.6 Hz, 1H), 8.30 (s, 1H), 8.24 (d, J = 7.8 Hz, 1H), 8.07 (d, J = 7.9 Hz, 1H), 7.76 (t, J = 7.8 Hz, 1H), 7.65 (s, 1H), 6.93 (s, 1H), 2.79 (t, J = 7.3 Hz, 4H), 1.91 (p, J = 7.4 Hz, 4H). 4H masked by DMSO peak, one exchangeable proton not observed. | m/z 424.1 (M + H)⁺ (ES+); 422.1 (M − H)⁻ (ES−) |
| | 3-((5-((1,2,3,5,6,7-hexahydro-s-indacen- | | |
| | 4-yl)amino)-4H-1,2,4-triazol-3- | | |
| | yl)sulfonyl)benzamide | | |
| 342 | 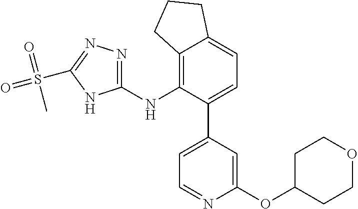 [SMILES: CS(=O)(=O)c1nnc(Nc2c(-c3ccnc(OC4CCOCC4)c3)ccc3c2CCC3)[nH]1] | ¹H NMR (DMSO-d₆) δ 9.10 (s, 1H), 8.06 (d, J = 5.3 Hz, 1H), 7.25 (d, J = 7.7 Hz, 1H), 7.19 (d, J = 7.6 Hz, 1H), 6.88 (dd, J = 5.3, 1.5 Hz, 1H), 6.71 (app s, 1H), 5.18-5.10 (m, 1H), 3.89-3.79 (m, 2H), 3.50- 3.43 (m, 2H), 3.13 (s, 3H), 2.95 (t, J = 7.4 Hz, 2H), 2.70 (t, J = 7.4 Hz, 2H), 2.06-1.93 (m, 4H), 1.64- 1.54 (m, 2H). One | m/z 456.3 (M + H)⁺ (ES⁺); 454.2 (M − H)⁻ (ES⁻) |
| | 5-(methylsulfonyl)-N-(5-(2- | exchangeable proton not | |
| | ((tetrahydro-2H-pyran-4- | observed. | |
| | yl)oxy)pyridin-4-yl)-2,3-dihydro-1H- | | |
| | inden-4-yl)-4H-1,2,4-triazol-3-amine | | |
| 343 | 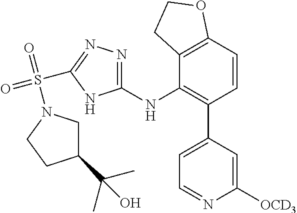 | ¹H NMR (DMSO-d₆) δ 13.09 (br s, 1H), 9.05 (br s, 1H), 8.07 (d, J = 5.3 Hz, 1H), 7.18 (d, J = 8.2 Hz, 1H), 6.89 (dd, J = 5.3, 1.3 Hz, 1H), 6.79 (d, J = 8.2 Hz, 1H), 6.72-6.67 (m, 1H), 4.58 (t, J = 8.7 Hz, 2H), 4.35 (s, 1H), 3.14- 3.06 (m, 2H), 3.01 (t, J = 8.7 Hz, 2H), 2.07-1.98 (m, 1H), 1.79-1.71 (m, 1H), 1.70-1.59 (m, 1H), 1.01 (d, J = 2.9 Hz, 6H). | m/z 504.3 (M + H)⁺ (ES⁺); 502.2 (M − H)⁻ (ES⁻) |
| | (S)-2-(1-((5-((5-(2-(methoxy-d₃)pyridin- | Two protons obscured by | |
| | 4-yl)-2,3-dihydrobenzofuran-4- | solvent. | |
| | yl)amino)-4H-1,2,4-triazol-3- | | |
| | yl)sulfonyl)pyrrolidin-3-yl)propan-2-ol | | |
| 344 | 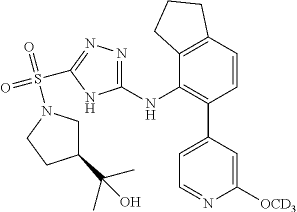 | ¹H NMR (DMSO-d₆) δ 13.00 (br s, 1H), 8.91 (s, 1H), 8.10 (d, J = 5.3 Hz, 1H), 7.26 (d, J = 7.7 Hz, 1H), 7.20 (d, J = 7.7 Hz, 1H), 6.91 (dd, J = 5.3, 1.4 Hz, 1H), 6.73 (br s, 1H), 4.35 (br s, 1H), 3.15-3.04 (m, 2H), 2.95 (t, J = 7.4 Hz, 2H), 2.69 (t, J = 7.4 Hz, 2H), 2.12-1.94 (m, 3H), 1.79-1.70 (m, 1H), 1.69-1.59 (m, 1H), 1.00 | m/z 502.4 (M + H)⁺ (ES⁺); 500.2 (M − H)⁻ (ES⁻) |
| | (S)-2-(1-((5-((5-(2-(methoxy-d₃)pyridin- | (d, J = 2.3 Hz, 6H). Two | |
| | 4-yl)-2,3-dihydro-1H-inden-4- | protons obscured by | |
| | yl)amino)-4H-1,2,4-triazol-3- | solvent. | |
| | yl)sulfonyl)pyrrolidin-3-yl)propan-2-ol | | |
| 345 | 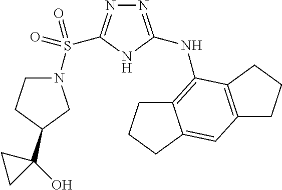 [SMILES: O=S(=O)(c1nnc(Nc2c3c(cc4c2CCC4)CCC3)[nH]1)N1CC[C@H](C2(O)CC2)C1] | ¹H NMR (DMSO-d₆) δ 13.02 (s, 1H), 8.89 (s, 1H), 6.95 (s, 1H), 5.23 (s, 1H), 3.49-3.40 (m, 2H), 3.30- 3.23 (m, 1H), 3.10 (app t, J = 9.7 Hz, 1H), 2.82 (t, J = 7.4 Hz, 4H), 2.62 (t, J = 7.2 Hz, 4H), 2.03-1.92 (m, 5H), 1.85-1.75 (m, 1H), 1.71-1.61 (m, 1H), 0.56-0.48 (m, 2H), 0.35- 0.29 (m, 2H). | m/z 430.3 (M + H)⁺ (ES⁺); 428.2 (M − H)⁻ (ES⁻) |
| | (S)-1-(1-((5-((1,2,3,5,6,7-hexahydro-s- | | |
| | indacen-4-yl)amino)-4H-1,2,4-triazol-3- | | |
| | yl)sulfonyl)pyrrolidin-3- | | |
| | yl)cyclopropanol | | |
| 346 | 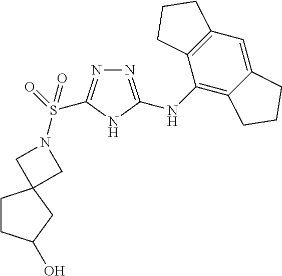 [SMILES: O=S(=O)(c1nnc(Nc2c3c(cc4c2CCC4)CCC3)[nH]1)N1CC2(CCC(O)C2)C1] | ¹H NMR (DMSO-d₆) δ 13.25 (br s, 1H), 8.97 (s, 1H), 6.97 (s, 1H), 4.46 (d, J = 3.6 Hz, 1H), 4.05- 4.01 (m, 1H), 3.86 (app d, J = 8.2 Hz, 1H), 3.80- 3.72 (m, 3H), 2.83 (t, J = 7.4 Hz, 4H), 2.66 (t, J = 7.3 Hz, 4H), 1.98 (p, J = 7.4 Hz, 4H), 1.81-1.70 (m, 2H), 1.70-1.63 (m, 1H), 1.61-1.51 (m, 2H), 1.45-1.37 (m, 1H). | m/z 430.1 (M + H)⁺ (ES⁺); 428.2 (M − H)⁻ (ES⁻) |
| | 2-((5-((1,2,3,5,6,7-hexahydro-s-indacen- | | |
| | 4-yl)amino)-4H-1,2,4-triazol-3- | | |
| | yl)sulfonyl)-2-azaspiro[3.4]octan-6-ol | | |
| 347 | 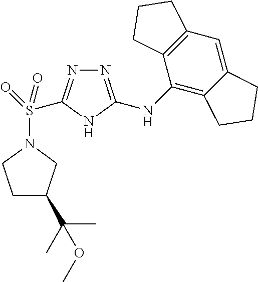 [SMILES: COC(C)(C)[C@H]1CCN(S(=O)(=O)c2nnc(Nc3c4c(cc5c3CCC5)CCC4)[nH]2)C1] | ¹H NMR (DMSO-d₆) δ 13.09 (s, 1H), 8.90 (s, 1H), 6.95 (s, 1H), 3.48-3.39 (m, 1H), 3.28-3.21 (m, 1H), 3.08 (app d, J = 10.0 Hz, 1H), 3.05 (s, 3H), 2.82 (t, J = 7.4 Hz, 4H), 2.63 (t, J = 7.4 Hz, 4H), 2.30- 2.15 (m, 1H), 1.97 (p, J = 7.4 Hz, 4H), 1.83-1.71 (m, 1H), 1.69-1.56 (m, 1H), 1.00 (d, J = 12.2 Hz, 6H). One proton obscured by solvent. | m/z 446.4 (M + H)⁺ (ES⁺); 444.3 (M − H)⁻ (ES⁻) |
| | (S)-N-(1,2,3,5,6,7-hexahydro-s-indacen- | | |
| | 4-yl)-5-((3-(2-methoxypropan-2- | | |
| | yl)pyrrolidin-1-yl)sulfonyl)-4H-1,2,4- | | |
| | triazol-3-amine | | |
| 348 | 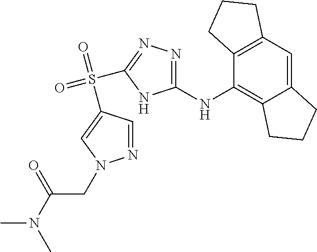 [SMILES: CN(C)C(=O)Cn1cc(S(=O)(=O)c2nnc(Nc3c4c(cc5c3CCC5)CCC4)[nH]2)cn1] | ¹H NMR (DMSO-d₆) δ 8.92 (s, 1H), 8.36 (s, 1H), 7.87 (s, 1H), 6.95 (s, 1H), 5.22 (s, 2H), 3.03 (s, 3H), 2.86 (s, 3H), 2.82 (t, J = 7.4 Hz, 4H), 2.59 (t, J = 7.4 Hz, 4H), 1.96 (p, J = 7.4 Hz, 4H). One exchangeable proton not observed. | m/z 456.2 (M + H)⁺ (ES⁺); 454.2 (M − H)⁻ (ES⁻) |
| | 2-(4-((5-((1,2,3,5,6,7-hexahydro-s- | | |
| | indacen-4-yl)amino)-4H-1,2,4-triazol-3- | | |
| | yl)sulfonyl)-1H-pyrazol-1-yl)-N,N- | | |
| | dimethylacetamide | | |
| 349 | 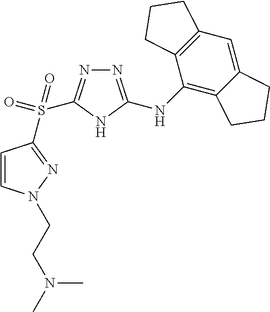 [SMILES: CN(C)CCn1ccc(S(=O)(=O)c2nnc(Nc3c4c(cc5c3CCC5)CCC4)[nH]2)n1] | ¹H NMR (DMSO-d₆) δ 13.15 (s, 1H), 8.97 (s, 1H), 8.05 (d, J = 2.4 Hz, 1H), 6.96 (s, 1H), 6.86 (d, J = 2.4 Hz, 1H), 4.48 (t, J = 6.2 Hz, 2H), 3.15 (br s, 2H), 2.82 (t, J = 7.4 Hz, 4H), 2.57 (t, J = 7.3 Hz, 4H), 2.48 (s, 6H), 1.95 (p, J = 7.4 Hz, 4H). | m/z 442.4 (M + H)⁺ (ES⁺); 440.3 (M − H)⁻ (ES⁻) |
| | 5-((1-(2-(dimethylamino)ethyl)-1H- | | |
| | pyrazol-3-yl)sulfonyl)-N-(1,2,3,5,6,7- | | |
| | hexahydro-s-indacen-4-yl)-4H-1,2,4- | | |
| | triazol-3-amine | | |
| 350 | 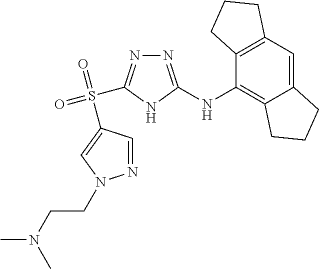 [SMILES: CN(C)CCn1cc(S(=O)(=O)c2nnc(Nc3c4c(cc5c3CCC5)CCC4)[nH]2)cn1] | ¹H NMR (DMSO-d₆) δ 9.01 (s, 1H), 8.49 (s, 1H), 7.87 (s, 1H), 6.95 (s, 1H), 4.26 (t, J = 6.3 Hz, 2H), 2.81 (t, J = 7.3 Hz, 4H), 2.66 (t, J = 6.3 Hz, 2H), 2.58 (t, J = 7.4 Hz, 4H), 2.15 (s, 6H), 1.95 (p, J = 7.4 Hz, 4H). One exchangeable proton not observed. | m/z 442.4 (M + H)⁺ (ES⁺); 440.3 (M − H)⁻ (ES⁻) |
| | 5-((1-(2-(dimethylamino)ethyl)-1H- | | |
| | pyrazol-4-yl)sulfonyl)-N-(1,2,3,5,6,7- | | |
| | hexahydro-s-indacen-4-yl)-4H-1,2,4- | | |
| | triazol-3-amine | | |
| 351 | 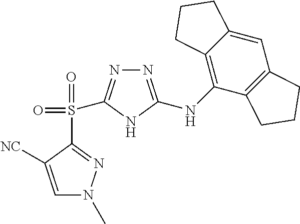 [SMILES: Cn1cc(C#N)c(S(=O)(=O)c2nnc(Nc3c4c(cc5c3CCC5)CCC4)[nH]2)n1] | ¹H NMR (DMSO-d₆) δ 13.47 (br s, 1H), 8.82 (br s, 1H), 8.37 (s, 1H), 6.91 (s, 1H), 4.10 (s, 3H), 2.80 (t, J = 7.4 Hz, 4H), 2.59- 2.52 (m, 4H), 1.93 (p, J = 7.4 Hz, 4H). | m/z 410.2 (M + H)⁺ (ES⁺); 408.2 (M − H)⁻ (ES⁻) |
| | 3-((5-((1,2,3,5,6,7-hexahydro-s-indacen- | | |
| | 4-yl)amino)-4H-1,2,4-triazol-3- | | |
| | yl)sulfonyl)-1-methyl-1H-pyrazole-4- | | |
| | carbonitrile | | |
| 352 | 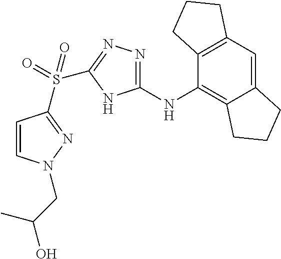 [SMILES: CC(O)Cn1ccc(S(=O)(=O)c2nnc(Nc3c4c(cc5c3CCC5)CCC4)[nH]2)n1] | ¹H NMR (DMSO-d₆) δ 8.99 (s, 1H), 7.91 (d, J = 2.4 Hz, 1H), 6.93 (s, 1H), 6.78 (d, J = 2.4 Hz, 1H), 4.17-4.11 (m, 1H), 4.09- 4.04 (m, 1H), 4.03-3.89 (m, 1H), 2.80 (t, J = 7.4 Hz, 4H), 2.59-2.52 (m, 4H), 1.94 (p, J = 7.4 Hz, 4H), 1.03 (d, J = 6.2 Hz, 3H). Two exchangeable protons not observed. | m/z 429.2 (M + H)⁺ (ES⁺) |
| | 1-(3-((5-((1,2,3,5,6,7-hexahydro-s- | | |
| | indacen-4-yl)amino)-4H-1,2,4-triazol-3- | | |
| | yl)sulfonyl)-1H-pyrazol-1-yl)propan-2-ol | | |
| 353 | 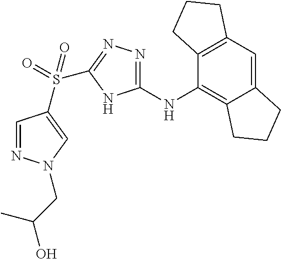 [SMILES: CC(O)Cn1cc(S(=O)(=O)c2nnc(Nc3c4c(cc5c3CCC5)CCC4)[nH]2)cn1] | ¹H NMR (DMSO-d₆) δ 8.19 (s, 1H), 7.71 (s, 1H), 7.57 (br s, 1H), 6.71 (s, 1H), 4.10-4.02 (m, 2H), 4.00-3.94 (m, 1H), 2.74 (t, J = 7.5 Hz, 4H), 2.63- 2.56 (m, 4H), 1.87 (p, J = 7.4 Hz, 4H), 1.03 (d, J = 6.1 Hz, 3H). Two exchangeable protons not observed. | m/z 429.3 (M + H)⁺ (ES⁺) |
| | 1-(4-((5-((1,2,3,5,6,7-hexahydro-s- | | |
| | indacen-4-yl)amino)-4H-1,2,4-triazol-3- | | |
| | yl)sulfonyl)-1H-pyrazol-1-yl)propan-2-ol | | |
| 354 | 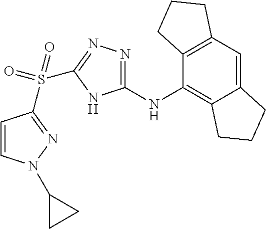 [SMILES: O=S(=O)(c1ccn(C2CC2)n1)c1nnc(Nc2c3c(cc4c2CCC4)CCC3)[nH]1] | ¹H NMR (DMSO-d₆) δ 8.92 (s, 1H), 8.05 (d, J = 2.4 Hz, 1H), 6.93 (s, 1H), 6.78 (d, J = 2.4 Hz, 1H), 3.93-3.85 (m, 1H), 2.80 (t, J = 7.4 Hz, 4H), 2.55 (t, J = 7.3 Hz, 4H), 1.94 (p, J = 7.4 Hz, 4H), 1.12-0.99 (m, 4H). One exchangeable proton not observed. | m/z 411.2 (M + H)⁺ (ES⁺) |
| | 5-((1-cyclopropyl-1H-pyrazol-3- | | |
| | yl)sulfonyl)-N-(1,2,3,5,6,7-hexahydro-s- | | |
| | indacen-4-yl)-4H-1,2,4-triazol-3-amine | | |
| 355 | 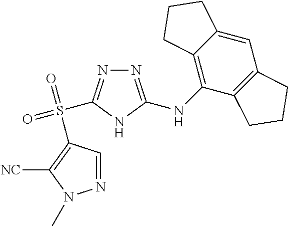 [SMILES: Cn1ncc(S(=O)(=O)c2nnc(Nc3c4c(cc5c3CCC5)CCC4)[nH]2)c1C#N] | ¹H NMR (DMSO-d₆) δ 13.18 (br s, 1H), 9.00 (br s, 1H), 8.23 (s, 1H), 6.95 (s, 1H), 4.09 (s, 3H), 2.81 (t, J = 7.4 Hz, 4H), 2.58 (t, J = 7.3 Hz, 4H), 1.95 (p, J = 7.4 Hz, 4H). | m/z 410.2 (M + H)⁺ (ES⁺); 432.2 (M + Na)⁺ (ES⁺); 408.2 (M − H)⁻ (ES⁻) |
| | 4-((5-((1,2,3,5,6,7-hexahydro-s-indacen- | | |
| | 4-yl)amino)-4H-1,2,4-triazol-3- | | |
| | yl)sulfonyl)-1-methyl-1H-pyrazole-5- | | |
| | carbonitrile | | |
| 356 | 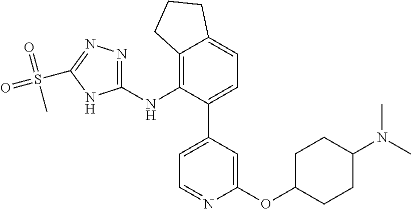 [SMILES: CN(C)C1CCC(Oc2cc(-c3ccc4c(c3Nc3nnc(S(C)(=O)=O)[nH]3)CCC4)ccn2)CC1] | ¹H NMR (DMSO-d₆) δ 9.10 (s, 1H), 8.02 (d, J = 5.3 Hz, 1H), 7.26 (d, J = 7.7 Hz, 1H), 7.19 (d, J = 7.7 Hz, 1H), 6.88 (dd, J = 5.3, 1.5 Hz, 1H), 6.67 (app br, s, 1H), 4.93-4.84 (m, 1H), 3.15 (s, 3H), 2.96 (t, J = 7.4 Hz, 2H), 2.88- 2.80 (m, 1H), 2.71 (t, J = 7.4 Hz, 2H), 2.54 (s, 6H), 2.19-2.11 (m, 2H), 2.03 (p, J = 7.5 Hz, 2H), 1.99-1.91 | m/z 497.4 (M + H)⁺ (ES⁺); 495.3 (M − H)⁻ (ES⁻) |
| | N-(5-(2-((4- | (m, 2H), 1.57-1.36 (m, | |
| | (dimethylamino)cyclohexyl)oxy)pyridin- | 4H). One exchangeable | |
| | 4-yl)-2,3-dihydro-1H-inden-4-yl)-5- | proton not observed. | |
| | (methylsulfonyl)-4H-1,2,4-triazol-3- | | |
| | amine | | |
| 357 | 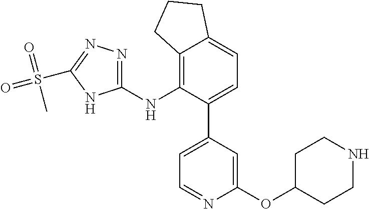 [SMILES: CS(=O)(=O)c1nnc(Nc2c(-c3ccnc(OC4CCNCC4)c3)ccc3c2CCC3)[nH]1] | ¹H NMR (DMSO-d₆) δ 9.17 (s, 1H), 8.31 (app s, 1H), 8.06 (d, J = 5.3 Hz, 1H), 7.23 (d, J = 7.7 Hz, 1H), 7.18 (d, J = 7.7 Hz, 1H), 6.91 (dd, J = 5.3, 1.5 Hz, 1H), 6.74 (br s, 1H), 5.16-5.08 (m, 1H), 3.19- 3.03 (m, 5H), 2.99-2.88 (m, 4H), 2.72 (t, J = 7.4 Hz, 2H), 2.09-1.97 (m, 4H), 1.78-1.66 (m, 2H). One exchangeable proton | m/z 455.3 (M + H)⁺ (ES⁺); 453.3 (M − H)⁻ (ES⁻) |
| | 5-(methylsulfonyl)-N-(5-(2-(piperidin- | not observed. | |
| | 4-yloxy)pyridin-4-yl)-2,3-dihydro-1H- | | |
| | inden-4-yl)-4H-1,2,4-triazol-3-amine | | |
| 358 | 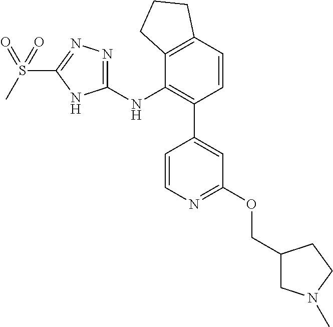 | ¹H NMR (DMSO-d₆) δ 9.05 (s, 1H), 8.08 (d, J = 5.3 Hz, 1H), 7.27 (d, J = 7.7 Hz, 1H), 7.20 (d, J = 7.6 Hz, 1H), 6.96-6.92 (m, 1H), 6.73 (br s, 1H), 4.29-4.13 (m, 2H), 3.15 (s, 3H), 3.10 (t, J = 9.5 Hz, 1H), 3.05-2.99 (m, 1H), 2.97 (t, J = 7.5 Hz, 3H), 2.86-2.79 (m, 1H), 2.73 (t, J = 7.4 Hz, 3H), 2.62 (s, 3H), 2.12-1.99 (m, 3H), 1.73-1.62 (m, 1H). One exchangeable proton not observed. | m/z 483.4 (M + H)⁺ (ES⁺); 481.2 (M − H)⁻ (ES⁻) |
| | N-(5-(2-((1-methylpyrrolidin-3- | | |
| | yl)methoxy)pyridin-4-yl)-2,3-dihydro- | | |
| | 1H-inden-4-yl)-5-(methylsulfonyl)-4H- | | |
| | 1,2,4-triazol-3-amine | | |
| 359 | 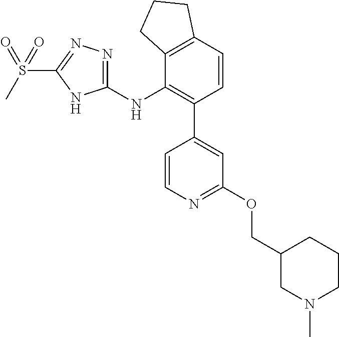 [SMILES: CN1CCCC(COc2cc(-c3ccc4c(c3Nc3nnc(S(C)(=O)=O)[nH]3)CCC4)ccn2)C1] | ¹H NMR (DMSO-d₆) δ 9.07 (s, 1H), 8.08 (d, J = 5.3 Hz, 1H), 7.27 (d, J = 7.7 Hz, 1H), 7.20 (d, J = 7.7 Hz, 1H), 6.91 (dd, J = 5.3, 1.5 Hz, 1H), 6.73 (br s, 1H), 4.22-4.14 (m, 1H), 4.12-4.03 (m, 1H), 3.14 (s, 3H), 3.02 (d, J = 10.5 Hz, 1H), 2.96 (t, J = 7.5 Hz, 2H), 2.93-2.86 (m, 1H), 2.72 (t, J = 7.5 Hz, 2H), 2.38 (s, 3H), 2.28- 2.20 (m, 1H), 2.20-2.00 (m, 4H), 1.76-1.67 (m, 2H), 1.63-1.49 (m, 1H), 1.19-1.02 (m, 1H). One exchangeable proton not observed. | m/z 483.4 (M + H)⁺ (ES⁺); 481.2 (M − H)⁻ (ES⁻) |
| | N-(5-(2-((1-methylpiperidin-3- | | |
| | yl)methoxy)pyridin-4-yl)-2,3-dihydro- | | |
| | 1H-inden-4-yl)-5-(methylsulfonyl)-4H- | | |
| | 1,2,4-triazol-3-amine | | |
| 360 | 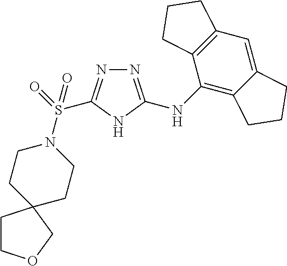 [SMILES: O=S(=O)(c1nnc(Nc2c3c(cc4c2CCC4)CCC3)[nH]1)N1CCC2(CCOC2)CC1] | ¹H NMR (DMSO-d₆) δ 8.92 (s, 1H), 6.96 (s, 1H), 3.72 (t, J = 7.1 Hz, 2H), 3.39 (s, 2H), 3.26-3.20 (m, 2H), 3.15-3.09 (m, 2H), 2.83 (t, J = 7.4 Hz, 4H), 2.64 (t, J = 7.3 Hz, 4H), 1.97 (p, J = 7.4 Hz, 4H), 1.64 (t, J = 7.1 Hz, 2H), 1.56 (t, J = 5.7 Hz, 4H). One exchangeable proton not observed. | m/z 444.3 (M + H)⁺ (ES⁺) |
| | 5-((2-oxa-8-azaspiro[4.5]decan-8- | | |
| | yl)sulfonyl)-N-(1,2,3,5,6,7-hexahydro-s- | | |
| | indacen-4-yl)-4H-1,2,4-triazol-3-amine | | |
| 361 | 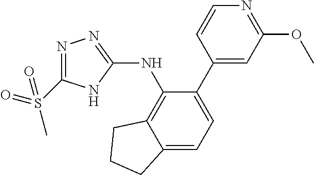 [SMILES: COc1cc(-c2ccc3c(c2Nc2nnc(S(C)(=O)=O)[nH]2)CCC3)ccn1] | ¹H NMR (DMSO-d₆) δ 13.09 (s, 1H), 9.02 (s, 1H), 8.11 (dd, J = 5.3, 0.7 Hz, 1H), 7.27 (d, J = 7.7 Hz, 1H), 7.21 (d, J = 7.7 Hz, 1H), 6.92 (dd, J = 5.3, 1.5 Hz, 1H), 6.73 (br s, 1H), 3.83 (s, 3H), 3.16 (s, 3H), 2.96 (t, J = 7.4 Hz, 2H), 2.71 (t, J = 7.4 Hz, 2H), 2.04 (p, J = 7.5 Hz, 2H). | m/z 386.1 (M + H)⁺ (ES⁺); 384.0 (M − H)⁻ (ES⁻) |
| | N-(5-(2-methoxypyridin-4-yl)-2,3- | | |
| | dihydro-1H-inden-4-yl)-5- | | |
| | (methylsulfonyl)-4H-1,2,4-triazol-3- | | |
| | amine | | |
| 362 | 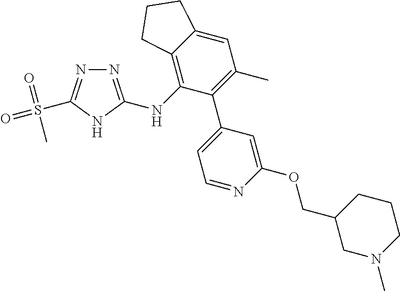 [SMILES: Cc1cc2c(c(Nc3nnc(S(C)(=O)=O)[nH]3)c1-c1ccnc(OCC3CCCN(C)C3)c1)CCC2] | ¹H NMR (DMSO-d₆) δ 8.65 (s, 1H), 8.11 (d, J = 5.2 Hz, 1H), 7.17 (s, 1H), 6.74 (dd, J = 5.2, 1.4 Hz, 1H), 6.55 (s, 1H), 4.25- 4.16 (m, 1H), 4.13-4.06 (m, 1H), 3.18 (s, 3H), 2.93 (t, J = 7.5 Hz, 2H), 2.70- 2.58 (m, 5H), 2.23-2.14 (m, 1H), 2.06-1.97 (m, 5H), 1.86-1.72 (m, 2H), 1.69-1.57 (m, 1H), 1.28- 1.13 (m, 2H). 1 × CH₃ masked by DMSO peak, one exchangeable proton not observed. | m/z 497.3 (M + H)⁺ (ES⁺) |
| | N-(6-methyl-5-(2-((1-methylpiperidin- | | |
| | 3-yl)methoxy)pyridin-4-yl)-2,3-dihydro- | | |
| | 1H-inden-4-yl)-5-(methylsulfonyl)-4H- | | |
| | 1,2,4-triazol-3-amine | | |
| 363 | 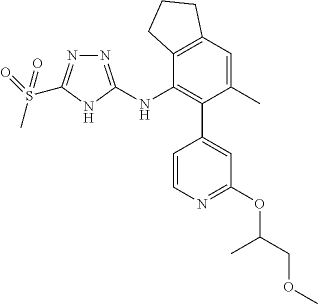 [SMILES: COCC(C)Oc1cc(-c2c(C)cc3c(c2Nc2nnc(S(C)(=O)=O)[nH]2)CCC3)ccn1] | ¹H NMR (DMSO-d₆) δ 12.96 (s, 1H), 8.67 (s, 1H), 8.10 (dd, J = 5.2, 0.7 Hz, 1H), 7.15 (s, 1H), 6.68 (dd, J = 5.2, 1.4 Hz, 1H), 6.51 (s, 1H), 5.37-5.21 (m, 1H), 3.56-3.48 (m, 1H), 3.48-340 (m, 1H), 3.27 (s, 3H), 3.17 (s, 3H), 2.92 (t, J = 7.4 Hz, 2H), 2.66- 2.59 (m, 2H), 2.07-1.92 (m, 5H), 1.23 (t, J = 5.7 Hz, 3H). | m/z 458.2 (M + H)⁺ (ES⁺) |
| | N-(5-(2-((1-methoxypropan-2-yl)oxy)- | | |
| | pyridin-4-yl)-6-methyl-2,3-dihydro-1H- | | |
| | inden-4-yl)-5-(methylsulfonyl)-4H- | | |
| | 1,2,4-triazol-3-amine | | |
| 364 | 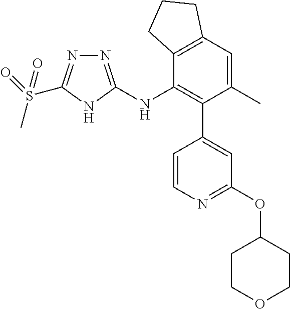 [SMILES: Cc1cc2c(c(Nc3nnc(S(C)(=O)=O)[nH]3)c1-c1ccnc(OC3CCOCC3)c1)CCC2] | ¹H NMR (DMSO-d₆) δ 12.96 (s, 1H), 8.67 (s, 1H), 8.10 (d, J = 5.2 Hz, 1H), 7.15 (s, 1H), 6.69 (dd, J = 5.2, 1.4 Hz, 1H), 6.53 (d, J = 1.2 Hz, 1H), 5.22-5.09 (m, 1H), 3.92-3.83 (m, 2H), 3.52-3.43 (m, 2H), 3.17 (s, 3H), 2.92 (t, J = 7.5 Hz, 2H), 2.63 (t, J = 7.3 Hz, 2H), 2.10-1.92 (m, 7H), 1.71-1.52 (m, 2H). | m/z 470.3 (M + H)⁺ (ES⁺) |
| | N-(6-methyl-5-(2-((tetrahydro-2H- | | |
| | pyran-4-yl)oxy)pyridin-4-yl)-2,3- | | |
| | dihydro-1H-inden-4-yl)-5- | | |
| | (methylsulfonyl)-4H-1,2,4-triazol-3- | | |
| | amine | | |
| 365 | 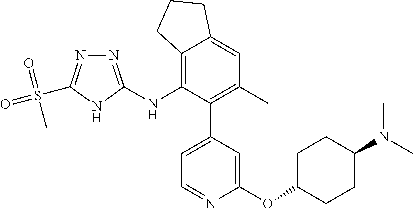 | ¹H NMR (DMSO-d₆) δ 8.69 (s, 1H), 8.09 (d, J = 5.2 Hz, 1H), 7.14 (s, 1H), 6.68 (dd, J = 5.2, 1.4 Hz, 1H), 6.48 (s, 1H), 4.92- 4.83 (m, 1H), 3.16 (s, 3H), 2.91 (t, J = 7.4 Hz, 2H), 2.77-2.68 (m, 1H), 2.66- 2.58 (m, 2H), 2.47 (s, 6H), 2.19-2.10 (m, 2H), 2.06-1.81 (m, 7H), 1.53- 1.32 (m, 4H). One exchangeable proton not | m/z 511.3 (M + H)⁺ (ES⁺) |
| | N-(5-(2-(((1r,4r)-4- | observed. | |
| | (dimethylamino)cyclohexyl)oxy)pyridin- | | |
| | 4-yl)-6-methyl-2,3-dihydro-1H-inden-4- | | |
| | yl)-5-(methylsulfonyl)-4H-1,2,4-triazol- | | |
| | 3-amine | | |
| 366 | 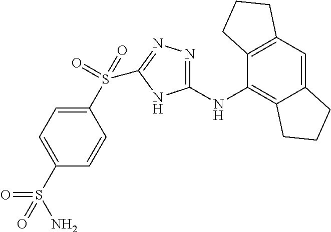 [SMILES: NS(=O)(=O)c1ccc(S(=O)(=O)c2nnc(Nc3c4c(cc5c3CCC5)CCC4)[nH]2)cc1] | ¹H NMR (DMSO-d₆) δ 8.84 (br s, 1H), 8.16- 8.09 (m, 2H), 8.09-8.03 (m, 2H), 7.64 (br s, 2H), 6.92 (s, 1H), 2.79 (t, J = 7.4 Hz, 4H), 1.92 (p, J = 7.4 Hz, 4H). 2 × CH₂ masked by DMSO peak, one exchangeable proton not observed. | m/z 460.2 (M + H)⁺ (ES+); 458.1 (M − H)⁻ (ES−) |
| | 4-((5-((1,2,3,5,6,7-hexahydro-s-indacen- | | |
| | 4-yl)amino)-4H-1,2,4-triazol-3- | | |
| | yl)sulfonyl)benzenesulfonamide | | |
| 367 | 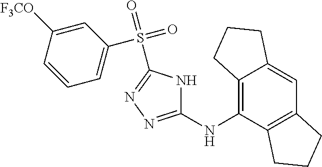 [SMILES: O=S(=O)(c1cccc(OC(F)(F)F)c1)c1nnc(Nc2c3c(cc4c2CCC4)CCC3)[nH]1] | ¹H NMR (400 MHz, CDCl₃) δ 8.02 (d, 1H), 7.92 (s, 1H), 7.62 (t, 1H), 7.53 (d, 1H), 7.44 (br s, 1H), 7.02 (s, 1H), 2.88 (t, 4H), 2.67 (t, 4H), 2.04- 1.97 (m, 4H). One exchangeable proton not observed. | m/z 465.1 (M + H)⁺ (ES⁺) |
| | N-(1,2,3,5,6,7-hexahydro-s-indacen-4- | | |
| | yl)-5-((3-(trifluoromethoxy)phenyl)- | | |
| | sulfonyl)-4H-1,2,4-triazol-3-amine | | |
| 368 | 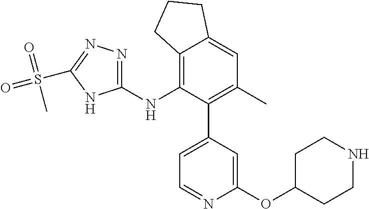 [SMILES: Cc1cc2c(c(Nc3nnc(S(C)(=O)=O)[nH]3)c1-c1ccnc(OC3CCNCC3)c1)CCC2] | ¹H NMR (DMSO-d₆) δ 8.65 (s, 1H), 8.10 (d, J = 5.2 Hz, 1H), 7.15 (s, 1H), 6.72 (dd, J = 5.2, 1.4 Hz, 1H), 6.59-6.52 (m, 1H), 5.18-5.11 (m, 1H), 3.21- 3.12 (m, 5H), 3.01-2.90 (m, 4H), 2.68-2.61 (m, 2H), 2.13-1.95 (m, 7H), 1.80-1.67 (m, 2H). Two exchangeable protons not observed. | m/z 469.3 (M + H)⁺ (ES⁺) |
| | N-(6-methyl-5-(2-(piperidin-4- | | |
| | yloxy)pyridin-4-yl)-2,3-dihydro-1H- | | |
| | inden-4-yl)-5-(methylsulfonyl)-4H- | | |
| | 1,2,4-triazol-3-amine | | |
| 369 | 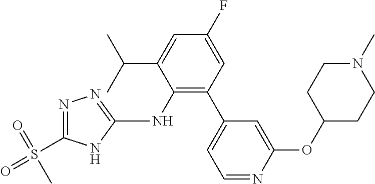 [SMILES: CC(C)c1cc(F)cc(-c2ccnc(OC3CCN(C)CC3)c2)c1Nc1nnc(S(C)(=O)=O)[nH]1] | ¹H NMR (DMSO-d₆) δ 9.02 (s, 1H), 8.07 (d, J = 5.3 Hz, 1H), 7.32 (dd, J = 10.0, 3.1 Hz, 1H), 7.13 (dd, J = 8.8, 3.0 Hz, 1H), 6.89 (dd, J = 5.3, 1.5 Hz, 1H), 6.73 (s, 1H), 4.96-4.88 (m, 1H), 3.19-3.12 (m, 1H), 3.10 (s, 3H), 2.71- 2.61 (m, 2H), 2.21 (s, 3H), 2.19-2.13 (m, 2H), 1.99- 1.89 (m, 2H), 1.69-1.59 | m/z 489.0 (M + H)⁺ (ES⁺) |
| | N-(4-fluoro-2-isopropyl-6-(2-((1- | (m, 2H), 1.16 (d, J = 6.9 | |
| | methylpiperidin-4-yl)oxy)pyridin-4- | Hz, 6H). One | |
| | yl)phenyl)-5-(methylsulfonyl)-4H-1,2,4- | exchangeable proton not | |
| | triazol-3-amine | observed. | |
| 370 | 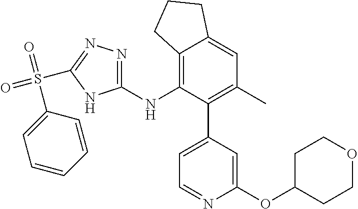 [SMILES: Cc1cc2c(c(Nc3nnc(S(=O)(=O)c4ccccc4)[nH]3)c1-c1ccnc(OC3CCOCC3)c1)CCC2] | ¹H NMR (DMSO-d₆) δ 8.54 (s, 1H), 7.99 (d, J = 5.2 Hz, 1H), 7.94-7.79 (m, 2H), 7.79-7.71 (m, 1H), 7.65 (t, J = 7.8 Hz, 2H), 7.12 (s, 1H), 6.60 (dd, J = 5.2, 1.4 Hz, 1H), 6.51-6.43 (m, 1H), 5.14- 5.06 (m, 1H), 3.85 (dt, J = 11.5, 4.2 Hz, 2H), 3.50- 3.43 (m, 2H), 2.89 (t, J = 7.4 Hz, 2H), 2.50-2.45 (m, 2H, obscured by | m/z 532.37 (M + H)⁺ (ES⁺) |
| | N-(6-methyl-5-(2-((tetrahydro-2H- | DMSO), 2.00 (s, 3H), 1.98- | |
| | pyran-4-yl)oxy)pyridin-4-yl)-2,3- | 1.89 (m, 4H), 1.64-1.51 | |
| | dihydro-1H-inden-4-yl)-5- | (m, 2H). One | |
| | (phenylsulfonyl)-4H-1,2,4-triazol-3- | exchangeable proton not | |
| | amine | observed. | |
| 371 | 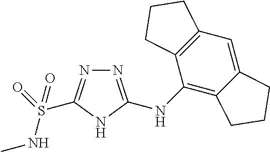 [SMILES: CNS(=O)(=O)c1nnc(Nc2c3c(cc4c2CCC4)CCC3)[nH]1] | ¹H NMR (DMSO-d₆) δ 12.90 (s, 1H), 8.84 (s, 1H), 7.94-7.45 (m, 1H), 6.95 (s, 1H), 2.82 (t, J = 7.4 Hz, 4H), 2.64 (t, J = 7.3 Hz, 4H), 2.57 (d, J = 4.9 Hz, 3H), 1.97 (p, J = 7.4 Hz, 4H). | m/z 334.1 (M + H)⁺ (ES⁺) |
| | 5-((1,2,3,5,6,7-hexahydro-s-indacen-4- | | |
| | yl)amino)-N-methyl-4H-1,2,4-triazole- | | |
| | 3-sulfonamide | | |
| 372 | 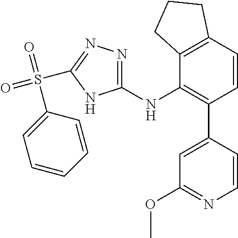 [SMILES: COc1cc(-c2ccc3c(c2Nc2nnc(S(=O)(=O)c4ccccc4)[nH]2)CCC3)ccn1] | ¹H NMR (DMSO-d₆) δ 13.11 (s, 1H), 8.97 (s, 1H), 8.00 (d, J = 5.3 Hz, 1H), 7.86 (d, J = 7.3 Hz, 2H), 7.77 (t, J = 7.4 Hz, 1H), 7.66 (t, J = 7.8 Hz, 2H), 7.25 (d, J = 7.7 Hz, 1H), 7.17 (d, J = 7.6 Hz, 1H), 6.83 (d, J = 5.3 Hz, 1H), 6.66 (s, 1H), 3.80 (s, 3H), 2.93 (t, J = 7.4 Hz, 2H), 2.56 (t, J = 7.5 Hz, 2H), 1.97 (p, J = 7.5 Hz, 2H). | m/z 448.1 (M + H)⁺ (ES⁺) |
| | N-(5-(2-methoxypyridin-4-yl)-2,3- | | |
| | dihydro-1H-inden-4-yl)-5- | | |
| | (phenylsulfonyl)-1H-1,2,4-triazol-3- | | |
| | amine | | |

### Example 373: 5-(((2-(diethylamino)ethyl)sulfonyl)methyl)-N-(1,2,3,5,6,7-hexahydro-s-indacen-4-yl)-4H-1,2,4-triazol-3-amine

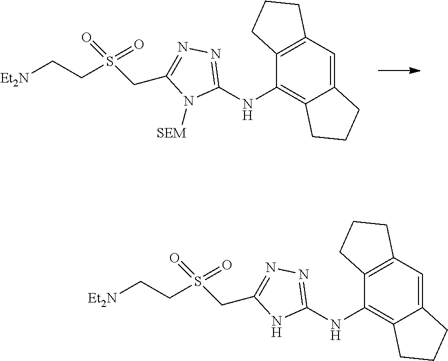

To a solution of 5-(((2-(diethylamino)ethyl)sulfonyl)methyl)-N-(1,2,3,5,6,7-hexahydro-s-indacen-4-yl)-((2-(trimethylsilyl)ethoxy)methyl)-4H-1,2,4-triazol-3-amine (Intermediate B57) (0.08 g, 146.03 μmol, 1 eq) in DCM (1 mL) was added TFA (1 mL). Then the reaction mixture was stirred at 25° C. for 0.5 hour. The mixture was concentrated in vacuum. The residue was purified by prep-HPLC (column: Phenomenex Synergi C18, 150 mm*25 mm*10 μm; mobile phase: [A: water (0.1% TFA v/v), B: MeCN]; B %: 15%-45%,10 minutes) to give the title compound (34.98 mg, 44.05% yield, 97.75% purity on HPLC, TFA salt) as a white solid.

¹H NMR (400 MHz, CDCl₃) δ 8.91 (br s, 1H), 7.02 (s, 1H), 4.50 (s, 2H), 3.87-3.82 (m, 2H), 3.57-3.53 (m, 2H), 3.16-3.11 (m, 4H), 2.87 (t, 4H), 2.74 (t, 4H), 2.10-1.98 (m, 4H), 1.24 (t, 6H). One exchangeable proton not observed. TFA proton not observed.

LCMS: m/z 418.2 (M+1)+(ES⁺).

### Example: 174: benzyl ((5-((1,2,3,5,6,7-hexahydro-s-indacen-4-yl)amino)-4H-1,2,4-triazol-3-yl)(oxo)(phenyl)-λ⁶-sulfaneylidene)carbamate

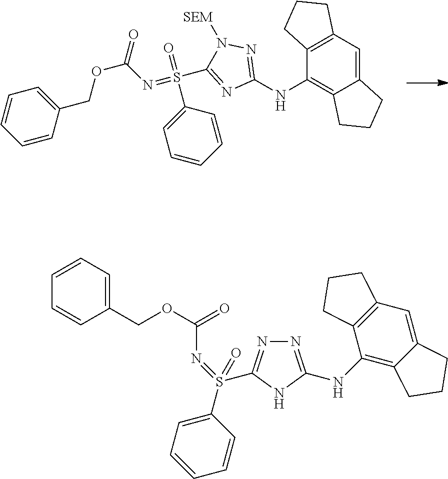 [SMILES: C[Si](C)(C)CCOCn1nc(Nc2c3c(cc4c2CCC4)CCC3)nc1S(=O)(=NC(=O)OCc1ccccc1)c1ccccc1 ; O=C(N=S(=O)(c1ccccc1)c1nnc(Nc2c3c(cc4c2CCC4)CCC3)[nH]1)OCc1ccccc1]

A mixture of benzyl ((3-((1,2,3,5,6,7-hexahydro-s-indacen-4-yl)amino)-1-((2-(trimethylsilyl)ethoxy)methyl)-1H-1,2,4-triazol-5-yl)(oxo)(phenyl)-6-sulfaneylidene)carbamate (Intermediate A17) (90 mg, 0.140 mmol) and TFA (0.5 mL) in DCM (4 mL) was stirred at RT for 1 h then evaporated. The residue was partitioned between DCM (30 mL) and sat. aq. NaHCO₃ (10 mL), the organic layer washed with water (10 mL), dried (MgSO₄), filtered and evaporated. The crude product was purified by chromatography on silica gel (24 g cartridge, 0-3% MeOH/DCM) to afford the title compound (30 mg, 38%) as a solid.

¹H NMR (CDCl₃) δ 6 8.23-8.17 (m, 2H), 7.88 (s, 1H), 7.72-7.66 (m, 1H), 7.60-7.54 (m, 2H), 7.26-7.19 (m, 5H), 6.99 (s, 1H), 5.13 (d, J=12.3 Hz, 1H), 5.00 (d, J=12.2 Hz, 1H), 2.87 (t, J=7.6 Hz, 4H), 2.75-2.53 (m, 4H), 2.03-1.89 (m, 4H). One exchangeable proton not observed.

### Example: 75: (5-((1,2,3,5,6,7-hexahydro-s-indacen-4-yl)amino)-4H-1,2,4-triazol-3-yl)(imino)(phenyl)-λ⁶-sulfanone

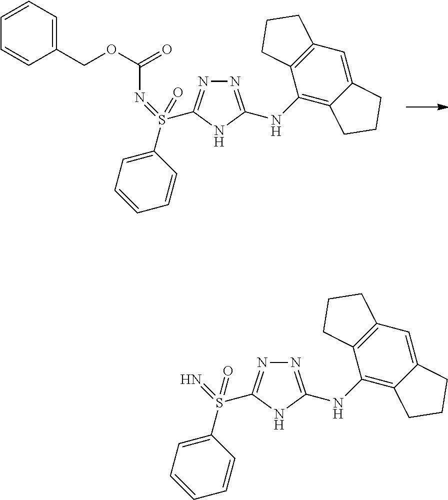 [SMILES: O=C(N=S(=O)(c1ccccc1)c1nnc(Nc2c3c(cc4c2CCC4)CCC3)[nH]1)OCc1ccccc1 ; N=S(=O)(c1ccccc1)c1nnc(Nc2c3c(cc4c2CCC4)CCC3)[nH]1]

A solution of benzyl ((5-((1,2,3,5,6,7-hexahydro-s-indacen-4-yl)amino)-4H-1,2,4-triazol-3-yl)(oxo)(phenyl)-λ⁶-sulfaneylidene)carbamate (Example 374) (25 mg, 0.049 mmol) in TFA (3 mL) was heated at 6° C. for 2 h. The mixture was evaporated and purified by acidic prep HPLC (50-80% MeOH in water) to afford the title (7 mg, 37%) as a white solid.

LCMS m/z 380.2 (M+H)⁺ (ES⁺), 378.2 (M−H)⁻ (ES⁻).

¹H NMR (DMSO-d₆) δ 12.91 (br s, 1H), 8.80 (s, 1H), 7.99 (d, J=7.1 Hz, 2H), 7.69-7.57 (m, 3H), 6.92 (s, 1H), 5.07 (s, 1H), 2.80 (t, J=7.4 Hz, 4H), 1.92 (p, J=7.4 Hz, 4H). 4H under DMSO peak.

### EXAMPLES—BIOLOGICAL STUDIES

NLRP and Pyroptosis

It is well established that the activation of NLRP3 leads to cell pyroptosis and this feature plays an important part in the manifestation of the clinical disease (Yan-gang Liu et al., Cell Death & Disease, 2017, 8(2), e2579; Alexander Wree et al., Hepatology, 2014, 59(3), 898-910; Alex Baldwin et al., Journal of Medicinal Chemistry, 2016, 59(5), 1691-1710; Ema Ozaki et al., Journal of Inflammation Research, 2015, 8, 15-27; Zhen Xie & Gang Zhao, Neuroimmunology Neuroinflammation, 2014, 1(2), 60-65; Mattia Cocco et al., Journal of Medicinal Chemistry, 2014, 57(24), 10366-10382; T. Satoh et al., Cell Death & Disease, 2013, 4, e644). Therefore, it is anticipated that inhibitors of NLRP3 will block pyroptosis, as well as the release of pro-inflammatory cytokines (e.g. IL-1β) from the cell.

THP-1 Cells: Culture and Preparation

THP-1 cells (ATCC #TIB-202) were grown in RPMI containing L-glutamine (Gibco #11835) supplemented with imM sodium pyruvate (Sigma #S8636) and penicillin (100 units/ml)/streptomycin (0.1 mg/ml) (Sigma #P4333) in 10% Fetal Bovine Serum (FBS) (Sigma #F0804). The cells were routinely passaged and grown to confluency (˜10⁶ cells/ml). On the day of the experiment, THP-1 cells were harvested and resuspended into RPMI medium (without FBS). The cells were then counted and viability (>90%) checked by Trypan blue (Sigma #T8154). Appropriate dilutions were made to give a concentration of 625,000 cells/ml. To this diluted cell solution was added LPS (Sigma #L4524) to give a 1 μg/ml Final Assay Concentration (FAC). 40 μl of the final preparation was aliquoted into each well of a 96-well plate. The plate thus prepared was used for compound screening.

THP-1 Cells Pyroptosis Assay

The following method step-by-step assay was followed for compound screening.
1. Seed THP-1 cells (25,000 cells/well) containing 1.0 μg/ml LPS in 40 μl of RPMI medium (without FBS) in 96-well, black walled, clear bottom cell culture plates coated with poly-D-lysine (VWR #734-0317)
2. Add 5 μl compound (8 points four-fold dilution or 8 points half-log dilution, with 10 μM top dose, unless stated otherwise) or vehicle (DMSO 0.1% FAC) to the appropriate wells
3. Incubate for 3 hrs at 37° C. and 5% CO₂
4. Add 5 μl nigericin (Sigma #N7143) (FAC 5 μM) to all wells
5. Incubate for 1 hr at 37° C. and 5% CO₂
6. At the end of the incubation period, spin plates at 300×g for 3 mins and remove supernatant
7. Then add 50 μl of resazurin (Sigma #R⁷⁰¹⁷) (FAC 100 μM resazurin in RPMI medium without FBS) and incubate plates for a further 1-2 hrs at 37° C. and 5% CO₂
8. Plates were read in an Envision reader at Ex 560 nm and Em 590 nm
9. IC₅₀ data is fitted to a non-linear regression equation (log inhibitor vs response-variable slope 4-parameters)

96-well Plate Map

| | 1 | 2 | 3 | 4 | 5 | 6 | 7 | 8 | 9 | 10 | 11 | 12 |
|---|---|---|---|---|---|---|---|---|---|---|---|---|
| A | High | Comp 1 | Comp 2 | Comp 3 | Comp 4 | Comp 5 | Comp 6 | Comp 7 | Comp 8 | Comp 9 | Comp 10 | Low |
| B | High | Comp 1 | Comp 2 | Comp 3 | Comp 4 | Comp 5 | Comp 6 | Comp 7 | Comp 8 | Comp 9 | Comp 10 | Low |
| C | High | Comp 1 | Comp 2 | Comp 3 | Comp 4 | Comp 5 | Comp 6 | Comp 7 | Comp 8 | Comp 9 | Comp 10 | Low |
| D | High | Comp 1 | Comp 2 | Comp 3 | Comp 4 | Comp 5 | Comp 6 | Comp 7 | Comp 8 | Comp 9 | Comp 10 | Low |
| E | High | Comp 1 | Comp 2 | Comp 3 | Comp 4 | Comp 5 | Comp 6 | Comp 7 | Comp 8 | Comp 9 | Comp 10 | Low |
| F | High | Comp 1 | Comp 2 | Comp 3 | Comp 4 | Comp 5 | Comp 6 | Comp 7 | Comp 8 | Comp 9 | Comp 10 | Low |
| G | High | Comp 1 | Comp 2 | Comp 3 | Comp 4 | Comp 5 | Comp 6 | Comp 7 | Comp 8 | Comp 9 | Comp 10 | Low |
| H | High | Comp 1 | Comp 2 | Comp 3 | Comp 4 | Comp 5 | Comp 6 | Comp 7 | Comp 8 | Comp 9 | Comp 10 | Low |

High MCC950 (10 uM)
Low Drug free control

The results of the pyroptosis assay performed are summarised in Table 1 below as THP IC₅₀.

Human Whole Blood IL1β Release Assay

For systemic delivery, the ability to inhibit NLRP3 when the compounds are present within the bloodstream is of great importance. For this reason, the NLRP3 inhibitory activity of a number of compounds in human whole blood was investigated in accordance with the following protocol.

Human whole blood in sodium-heparin or lithium-heparin tubes was obtained from healthy donors from a volunteer donor panel.
1. Plate out 80 μl of whole blood containing 1 μg/ml of LPS in 96-well, clear bottom cell culture plate (Corning #3585)
2. Add 10 μl compound (8 points half-log dilution with 10 μM top dose, unless stated otherwise) or vehicle (DMSO 0.1% FAC) to the appropriate wells
3. Incubate for 3 hrs at 37° C., 5% CO₂
4. Add 10 μl nigericin (Sigma #N7143) (10 μM FAC) to all wells
5. Incubate for 1 hr at 37° C., 5% CO₂
6. At the end of the incubation period, spin plates at 300×g for 5 mins to pellet cells and remove 20 μl of supernatant and add to 96-well v-bottom plates for IL-1β analysis (note: these plates containing the supernatants can be stored at −80° C. to be analysed at a later date)
7. IL-1β was measured according to the manufacturer protocol (Perkin Elmer-AlphaLisa IL-1 Kit AL220F-5000)
8. IC₅₀ data is fitted to a non-linear regression equation (log inhibitor vs response-variable slope 4-parameters)

The results of the human whole blood assay are summarised in Table 1 below as HWB IC₅₀.

TABLE 1
NLRP3 inhibitory activity (≤0.5 μM = ‘+++++’, ≤1 μM = ‘++++’,
≤5 μM = ‘+++’, ≤10 μM = ‘++’, ≤30 μM = ‘+’ not
determined = ‘ND’). Example 2 was screened against a top dose
of 30 μM for both the THP and HWB assay.

| Example No | THP IC₅₀ | HWB IC₅₀ |
|---|---|---|
| 2 | ++ | + |
| 5 | +++++ | ++ |
| 8 | ++++ | ND |
| 12 | ++++ | ++ |
| 14 | +++ | ND |
| 16 | +++ | ND |
| 18 | +++++ | ++ |
| 21 | ++ | ND |
| 22 | +++ | ++ |
| 24 | +++++ | ++++ |
| 25 | ++ | ND |
| 26 | ++ | ND |
| 28 | ++ | ND |
| 30 | ++ | ND |
| 32 | +++ | ND |
| 34 | ++++ | +++ |
| 36 | +++ | ++ |
| 39 | ++ | ND |
| 40 | ++++ | ND |
| 41 | ++ | ND |
| 42 | ++ | ND |
| 43 | ++++ | ND |
| 44 | +++++ | +++ |
| 45 | +++ | +++ |
| 46 | ++++ | +++ |
| 47 | +++ | ND |
| 48 | +++++ | +++++ |
| 49 | +++++ | ++++ |
| 50 | +++ | ND |
| 51 | +++ | ND |
| 52 | +++ | ND |
| 53 | +++ | ND |
| 54 | ++++ | ++ |
| 55 | +++ | ND |
| 56 | +++ | ND |
| 57 | ++ | ND |
| 58 | +++ | ND |
| 59 | +++++ | ++++ |
| 60 | ++++ | +++ |
| 61 | ++ | ND |
| 62 | ++ | ND |
| 63 | ++++ | ND |
| 64 | +++ | ND |
| 65 | ++ | ND |
| 66 | ++ | ND |
| 67 | ++ | ND |
| 68 | +++ | ND |
| 69 | ++++ | ++++ |
| 70 | +++ | ND |
| 71 | ++ | ND |
| 72 | ++ | ND |
| 73 | ++ | ND |
| 74 | +++ | ND |
| 75 | +++ | ++ |
| 76 | +++++ | +++ |
| 77 | ++++ | +++ |
| 78 | ++ | ND |
| 79 | +++++ | +++ |
| 80 | ++++ | +++ |
| 81 | +++++ | ++++ |
| 82 | ++ | ND |
| 83 | ++ | ND |
| 84 | +++ | ND |
| 85 | ++ | ND |
| 86 | +++ | ND |
| 87 | +++ | ++++ |
| 88 | +++ | ND |
| 89 | ++++ | +++ |
| 90 | ++ | ND |
| 91 | +++ | +++ |
| 92 | ++ | ND |
| 93 | +++ | +++ |
| 94 | ++++ | ++++ |
| 95 | ++++ | +++ |
| 96 | +++ | ND |
| 97 | +++ | ND |
| 98 | +++ | ND |
| 99 | ++ | ND |
| 100 | +++ | ND |
| 101 | +++ | ND |
| 102 | ++ | ND |
| 103 | +++ | ND |
| 104 | +++++ | +++ |
| 105 | +++ | ND |
| 106 | ++ | ND |
| 107 | ++ | ND |
| 108 | +++++ | +++ |
| 109 | +++++ | ++++ |
| 110 | +++++ | ++ |
| 111 | ++ | ND |
| 112 | +++ | ND |
| 113 | +++ | ND |
| 114 | +++ | ND |
| 115 | +++ | ND |
| 116 | +++ | ND |
| 117 | ++++ | +++ |
| 118 | +++++ | +++++ |
| 119 | +++++ | +++++ |
| 120 | +++ | +++ |
| 121 | ++ | ND |
| 122 | +++ | ++ |
| 123 | +++ | ND |
| 124 | ++++ | +++ |
| 125 | +++++ | +++ |
| 126 | ++++ | +++ |
| 127 | +++ | ND |
| 128 | ++++ | ++ |
| 129 | +++ | ND |
| 130 | ++++ | +++ |
| 131 | +++ | ND |
| 132 | +++++ | ++ |
| 133 | +++++ | ++ |
| 134 | +++ | ND |
| 135 | +++ | ND |
| 136 | +++++ | +++ |
| 137 | +++++ | +++++ |
| 138 | +++ | +++ |
| 139 | ++++ | ++ |
| 140 | +++ | ND |
| 141 | +++ | ++ |
| 142 | +++ | ND |
| 143 | +++++ | +++++ |
| 144 | +++++ | +++++ |
| 145 | ++++ | +++ |
| 146 | +++ | ++ |
| 147 | +++++ | +++++ |
| 148 | +++++ | +++++ |
| 149 | ++++ | +++ |
| 150 | +++ | +++ |
| 151 | +++ | +++ |
| 152 | +++ | +++ |
| 153 | +++++ | +++ |
| 155 | ++ | ND |
| 156 | ++++ | +++ |
| 157 | ++ | ND |
| 158 | +++ | ND |
| 159 | +++ | ND |
| 160 | +++ | ND |
| 161 | +++ | ND |
| 167 | +++ | ND |
| 168 | +++ | ND |
| 169 | ++ | ND |
| 170 | ++++ | ND |
| 171 | +++ | ND |
| 172 | ++++ | ND |
| 173 | ++++ | ND |
| 174 | +++ | ND |
| 175 | +++ | ND |
| 176 | ++++ | ++ |
| 177 | +++ | ND |
| 178 | +++ | ND |
| 179 | ++++ | ++ |
| 180 | +++ | ND |
| 181 | +++ | ND |
| 182 | +++ | ND |
| 183 | +++ | ND |
| 184 | +++ | ND |
| 185 | +++++ | ND |
| 186 | +++++ | ND |
| 187 | ++++ | ND |
| 188 | +++ | ND |
| 189 | ++ | ND |
| 190 | +++ | ND |
| 191 | ++ | ND |
| 192 | ++++ | +++ |
| 193 | +++ | ND |
| 194 | ++ | ND |
| 195 | +++++ | +++++ |
| 196 | +++++ | +++++ |
| 197 | +++++ | +++++ |
| 198 | ++ | ND |
| 199 | +++++ | +++ |
| 200 | +++++ | +++ |
| 201 | +++++ | +++++ |
| 202 | +++++ | ++++ |
| 203 | +++++ | +++ |
| 204 | +++++ | ++++ |
| 205 | ++ | ND |
| 206 | ++ | ND |
| 207 | +++ | ND |
| 208 | ++ | ND |
| 209 | ++ | ND |
| 210 | +++++ | +++ |
| 211 | +++++ | +++ |
| 212 | +++++ | +++++ |
| 213 | +++++ | ND |
| 214 | +++++ | +++++ |
| 215 | +++++ | +++++ |
| 216 | +++++ | ++++ |
| 217 | +++ | ++ |
| 218 | +++++ | +++++ |
| 219 | +++++ | ++++ |
| 220 | ++++ | +++ |
| 221 | +++++ | +++ |
| 222 | +++++ | ++++ |
| 223 | +++ | ND |
| 224 | +++++ | ++++ |
| 225 | +++++ | ++++ |
| 226 | +++++ | ++++ |
| 227 | +++++ | +++++ |
| 228 | +++++ | ++++ |
| 229 | ++ | ND |
| 230 | +++ | ND |
| 231 | +++++ | ++++ |
| 232 | ++++ | ND |
| 233 | +++++ | +++ |
| 234 | +++ | ND |
| 235 | ++ | ND |
| 236 | ++ | ND |
| 237 | +++ | ND |
| 238 | ++ | ND |
| 239 | ++++ | +++ |
| 240 | +++++ | +++++ |
| 241 | +++++ | ++++ |
| 242 | +++++ | +++++ |
| 243 | +++ | ND |
| 244 | +++ | ND |
| 245 | +++++ | +++ |
| 246 | ++++ | +++ |
| 247 | +++++ | +++++ |
| 248 | +++++ | ++++ |
| 249 | +++++ | +++ |
| 250 | +++++ | +++ |
| 251 | +++++ | +++ |
| 252 | ++++ | +++ |
| 253 | +++ | ND |
| 254 | +++++ | +++ |
| 255 | ++++ | ND |
| 256 | +++++ | ND |
| 257 | +++++ | +++++ |
| 258 | +++++ | ++++ |
| 259 | +++++ | ++++ |
| 260 | +++++ | +++ |
| 261 | +++++ | ++++ |
| 262 | +++ | ND |
| 263 | +++ | ND |
| 264 | +++++ | +++++ |
| 265 | +++ | ND |
| 266 | +++ | ND |
| 267 | ++ | ND |
| 268 | +++++ | ++++ |
| 269 | ++ | ND |
| 270 | +++++ | +++++ |
| 271 | +++++ | ++++ |
| 272 | ++ | ND |
| 273 | ++ | ND |
| 274 | +++++ | +++++ |
| 275 | +++ | ++++ |
| 276 | +++++ | +++++ |
| 277 | +++++ | ++++ |
| 278 | +++++ | +++++ |
| 279 | +++++ | +++++ |
| 280 | +++ | ND |
| 281 | ++ | ND |
| 282 | +++ | ND |
| 283 | +++ | ND |
| 284 | ++ | ND |
| 285 | +++ | ND |
| 286 | +++ | ++ |
| 287 | ++ | ND |
| 288 | +++++ | ++++ |
| 289 | ++++ | +++ |
| 290 | +++ | +++ |
| 291 | +++++ | +++++ |
| 292 | +++ | +++ |
| 293 | +++++ | ++ |
| 294 | +++++ | +++ |
| 295 | +++++ | ++++ |
| 296 | +++++ | ND |
| 297 | ++ | ND |
| 298 | ++ | ND |
| 299 | +++++ | +++++ |
| 300 | +++++ | +++ |
| 301 | +++++ | +++ |
| 302 | +++ | ND |
| 303 | ++++ | +++ |
| 304 | +++++ | ++++ |
| 305 | +++ | +++ |
| 306 | ++++ | ++++ |
| 307 | ++++ | ++ |
| 308 | +++++ | +++ |
| 309 | +++ | ND |
| 310 | +++ | ND |
| 311 | +++ | ND |
| 312 | +++ | ND |
| 313 | +++ | ND |
| 314 | +++ | ND |
| 315 | +++ | ND |
| 316 | +++ | ND |
| 317 | ++++ | +++ |
| 318 | +++++ | +++ |
| 319 | +++++ | ++ |
| 320 | +++++ | +++ |
| 321 | +++ | ND |
| 322 | +++ | ND |
| 323 | +++ | ND |
| 324 | +++++ | ND |
| 325 | +++++ | ++ |
| 326 | +++ | ND |
| 327 | +++ | ND |
| 328 | +++++ | ++ |
| 329 | +++++ | +++ |
| 330 | ++++ | ++ |
| 331 | +++ | +++ |
| 332 | ++ | ND |
| 333 | +++ | ND |
| 334 | ++++ | ND |
| 335 | +++ | ND |
| 336 | +++ | ND |
| 337 | +++++ | ND |
| 338 | +++ | ND |
| 339 | ++++ | +++ |
| 340 | +++ | ND |
| 341 | +++++ | ++ |
| 342 | +++ | ND |
| 343 | +++++ | ++++ |
| 344 | +++++ | +++ |
| 345 | ++++ | ++ |
| 346 | ++++ | +++ |
| 347 | ++++ | ++ |
| 348 | +++ | ND |
| 349 | +++++ | +++ |
| 350 | ++++ | ND |
| 351 | +++ | ND |
| 352 | +++++ | ++ |
| 353 | +++ | ND |
| 354 | +++++ | ND |
| 355 | +++ | ND |
| 356 | +++ | ND |
| 357 | ++ | ND |
| 358 | ++ | ND |
| 359 | +++ | ND |
| 360 | +++++ | +++ |
| 361 | ++ | ND |
| 362 | +++ | ND |
| 363 | +++ | ND |
| 364 | +++ | ND |
| 365 | +++ | ND |
| 366 | +++++ | ++ |
| 367 | +++ | ND |
| 368 | +++ | ND |
| 369 | +++ | ND |
| 370 | +++++ | +++ |
| 371 | +++ | ND |
| 372 | +++ | ND |
| 373 | ++ | ND |

PK Protocol

Pharmacokinetic parameters were determined in male Sprague Dawley rats (Vital River Laboratory Animal Technology Co Ltd, Beijing, China, 6-9 weeks old). Animals were maintained under a 12 h light/dark cycle. Animals had free access to food and water, except that orally dosed animals were food deprived overnight prior to the study.

For intravenous administration, compounds were formulated as a solution (in DMSO:water [10:90] or in DMSO:PBS [10:90]) in 2 mL/kg dosing volume and administered via tail vein. For oral administration, compounds were formulated as a solution (in 0.5% methylcellulose in water) in mL/kg dosing volume and administered orally.

Serial blood samples (about 200 μL) were taken from each animal at each of 8 time-points post dose (0.83, 0.25, 0.5, 1, 2, 4, 8 and 24 h). Samples were held on ice for no longer than 30 minutes before centrifugation (5,696 rpm (3,000 g) for 15 minutes) for plasma generation. Plasma was frozen on dry ice prior to bioanalysis. PK parameters were generated from LC-MS/MS data using Phoenix WinNonlin 6.3 software.

TABLE 2
PK data (intravenous administration)

| Example | Dose | AUC | T_{1/2} | V_{dss} | Cl |
|---|---|---|---|---|---|
| No | (mg/kg) | (ng · hr/mL) | (hr) | (L/kg) | (mL/min/kg) |
| 22 | 1 | 363.9 | 0.4 | 1.09 | 45.8 |
| 24 | 1 | 172.8 | 0.8 | 5.26 | 96.5 |
| 40 | 1 | 1459.1 | 0.9 | 0.54 | 11.4 |
| 43 | 1 | 601.6 | 0.7 | 0.66 | 28.0 |
| 45 | 1 | 544.8 | 1.2 | 1.59 | 30.6 |
| 48 | 1 | 196.1 | 0.8 | 3.69 | 85.0 |
| 49 | 1 | 168.4 | 2.1 | 8.08 | 98.9 |
| 54 | 1 | 272.8 | 1.3 | 2.7 | 61.1 |
| 60 | 1 | 277.9 | 1.0 | 3.24 | 60.0 |
| 69 | 1.05 | 267 | 0.71 | 1.29 | 65.5 |
| 75 | 1 | 1001.0 | 0.5 | 0.56 | 16.7 |
| 76 | 1 | 210.0 | 0.9 | 2.48 | 79.4 |
| 79 | 1.18 | 277 | 2.57 | 5.03 | 60 |
| 81 | 1 | 278.3 | 1.5 | 4.61 | 59.9 |
| 94 | 1 | 353.8 | 1.3 | 3.21 | 47.1 |
| 118 | 1 | 307.4 | 1.4 | 4.01 | 54.2 |
| 119 | 1 | 162.4 | 2.6 | 10.09 | 106.0 |
| 130 | 1 | 508.9 | 7.8 | 10.63 | 32.8 |
| 153 | 0.993 | 198 | 0.36 | 2.66 | 84.1 |
| 156 | 1.19 | 193 | 0.4 | 2.95 | 103 |
| 159 | 0.93 | 2250 | 0.93 | 0.38 | 6.9 |
| 176 | 0.93 | 814 | 0.473 | 0.41 | 19.1 |
| 212 | 0.985 | 216 | 0.19 | 0.88 | 76.1 |
| 213 | 1.01 | 186 | 0.73 | 4.21 | 90.6 |
| 214 | 0.85 | 182 | 0.25 | 1.02 | 78.1 |
| 215 | 0.42 | 58 | 0.52 | 2.86 | 121 |
| 216 | 0.971 | 224 | 2.18 | 6.41 | 72.3 |
| 226 | 1.2 | 216 | 0.78 | 1.48 | 77.2 |
| 231 | 1.02 | 459 | 0.326 | 0.494 | 36.3 |
| 235 | 1.01 | 430 | 2.16 | 0.935 | 38.7 |
| 236 | 0.937 | 3159 | 0.891 | 0.149 | 5.28 |
| 245 | 1.17 | 393 | 0.74 | 1.63 | 49.6 |
| 247 | 1.1 | 253 | 2.72 | 12.7 | 72.5 |
| 250 | 1.3 | 392 | 0.26 | 0.6 | 55.2 |
| 252 | 1.11 | 362 | 0.426 | 0.82 | 51.1 |
| 259 | 0.92 | 169 | 2.23 | 9.57 | 90.1 |
| 264 | 0.994 | 280 | 1.89 | 5.78 | 59.6 |
| 268 | 1.06 | 175 | 2.52 | 12.1 | 95.3 |
| 270 | 1.09 | 236 | 2.53 | 9.16 | 71 |
| 271 | 1.11 | 221 | 3.53 | 14.9 | 75 |
| 274 | 1.04 | 271 | 1.64 | 6.2 | 61.6 |
| 276 | 1.06 | 239 | 1.94 | 7.51 | 69.7 |
| 278 | 1.11 | 278 | 1.8 | 5.9 | 60 |
| 296 | 1.01 | 315 | 0.59 | 1.7 | 52.9 |
| 299 | 1.21 | 195 | 1.33 | 5.21 | 103 |
| 300 | 1.02 | 235 | 2.65 | 10.6 | 70.8 |
| 302 | 1.08 | 153 | 1.34 | 4.03 | 109 |
| 303 | 0.97 | 200 | 2.45 | 11.5 | 80.7 |
| 310 | 1 | 221 | 0.458 | 1.45 | 75.4 |
| 328 | 1.05 | 1239 | 0.653 | 0.518 | 13.5 |
| 337 | 1.11 | 981 | 0.595 | 0.54 | 17 |

TABLE 3
PK data (oral administration)

| Example | Dose | Cₘₐₓ | AUC | Tₘₐₓ | T_{1/2} | Cl/F | Bioavailability |
|---|---|---|---|---|---|---|---|
| No | (mg/kg) | (ng/mL) | (ng · hr/mL) | (hr) | (hr) | (mL/min/kg) | (%) |
| 43 | 3 | 593.5 | 877.8 | 0.25 | 1.2 | 61.5 | 48.6 |

As is evident from the results presented in Table 1, surprisingly in spite of the structural differences versus the prior art compounds, the compounds of the invention show high levels of NLRP3 inhibitory activity in the pyroptosis assay and in the human whole blood assay.

As is evident from the results presented in Tables 2 and 3, the compounds of the invention show advantageous pharmacokinetic properties, for example half-life T_{1/2}, area under the curve AUC, clearance C1 and/or bioavailability, compared to the prior art compounds.

It will be understood that the present invention has been described above by way of example only. The examples are not intended to limit the scope of the invention. Various modifications and embodiments can be made without departing from the scope and spirit of the invention, which is defined by the following claims only.

1. A compound of formula (I): 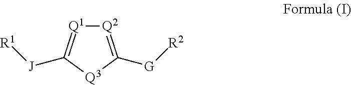
wherein:
Q¹ and Q² are each independently selected from N or CR^{q}, provided that at least one of Q¹ and Q² is N;
Q³ is O, S or NR^{qq};
each R^{q} is independently selected from hydrogen or a halo, —OH, —NO₂, —NH₂, —N₃, —SH, —SO₂H, —SO₂NH₂, or a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton;
each R^{qq} is independently selected from hydrogen or a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton;
J is a bond, —O—, —S—, —SO—, —SO₂—, —SO(═NR^{jj})—, —CO—, —C(═S)—, —C(R^{j})₂—, —C(═C(R^{jjj})₂)—, —C(═NR^{jj})—, —NR^{jj}—, —O—C(R^{j})₂—, —O—C(═C(R^{jjj})₂)—, —O—C(═NR^{jj})—, —S—C(R^{j})₂—, —S—C(═C(R^{jjj})₂)—, —S—C(═NR^{jj})—, —SO—C(R^{j})₂—, —SO—C(═C(R^{jjj})₂)—, —SO—C(═NR^{jj})—, —SO—NR^{jj}—, —SO₂—C(R^{j})₂—, —SO₂—C(═C(R^{jjj})₂)—, —SO₂—C(═NR^{jj})—, —SO₂—NR^{jj}—, —SO(═NR^{jj})—C(R^{j})₂—, —SO(═NR^{jj})—C(═C(R^{jjj})₂)—, —SO(═NR^{jj})—NR^{jj}—, —CO—C(R^{j})₂—, —CO—C(═C(R^{jjj})₂)—, —CO—C(═NR^{jj})—, —CO—NR^{jj}—, —C(═S)—C(R^{j})₂—, —C(═S)—C(═C(R^{jjj})₂)—, —C(R^{j})₂—O—, —C(R^{j})₂—S—, —C(R^{j})₂—SO—, —C(R^{j})₂—SO₂—, —C(R^{j})₂—SO(═NR^{jj})—, —C(R^{j})₂—CO—, —C(R^{j})₂—C(═S)—, —C(R^{j})₂—C(R^{j})₂—, —C(R^{j})₂—C(═C(R^{jjj})₂)—, —C(R^{j})₂—C(═NR^{jj})—, —C(R^{j})₂—NR^{jj}—, —C(═C(R^{jjj})₂)—O—, —C(═C(R^{jjj})₂)—S—, —C(═C(R^{jjj})₂)—SO—, —C(═C(R^{jjj})₂)—SO₂—, —C(═C(R^{jjj})₂)—SO(═NR^{jj})—, —C(═C(R^{jj})₂)—CO—, —C(═C(R^{jjj})₂)—C(═S)—, —C(═C(R^{jjj})₂)—C(R^{j})₂—, —C(═C(R^{jjj})₂)—C(═C(R^{jjj})₂)—, —C(═C(R^{jjj})₂)—C(═NR^{jj})—, —C(═C(R^{jjj})₂)—NR^{jj}—, —C(═NR^{jj})—O—, —C(═NR^{jj})—S—, —C(═NR^{jj})—SO—, —C(═NR^{jj})—SO₂—, —C(═NR^{jj})—CO—, —C(═NR^{jj})—C(R^{j})₂—, —C(═NR^{jj})—C(═C(R^{jjj})₂)—, —C(═NR^{jj})—C(═NR^{jj})—, —C(═NR^{jj})—NR^{jj}—, —NR—SO—, —NR^{jj}—SO₂—, —NR^{jj}—SO(═NR^{jj})—, —NR^{jj}—CO—, —NR—C(R^{jj})₂—, —NR^{jj}—C(═C(R^{jjj})₂)—, —NR^{jj}—C(═NR^{jj})—, —CR^{jjj}═CR^{jjj}—, —CR^{jjj}═N—, —N═CR^{jjj}— or —C≡C—;
each R^{j} is independently selected from hydrogen or a halo, —OH, —NO₂, —NH₂, —N₃, —SH, —SO₂H, —SO₂NH₂, or a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton;
each R^{jj} is independently selected from hydrogen or a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton;
each R^{jjj} is independently selected from hydrogen or a halo or a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton;
or wherein optionally any two or three R^{j}, any two R^{jj}, any two or three R^{jjj}, or any two or three of R^{j}, R^{jj} and R^{jjj}, together with the atom or atoms to which they are attached, may form a saturated or unsaturated cyclic group, wherein the cyclic group may optionally be substituted;
R¹ is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton;
G is a bond, —O—, —S—, —SO—, —SO₂—, —SO(═NR^{gg})—, —CO—, —C(═S)—, —C(R^{g})₂—, —C(═C(═C(R^{ggg})₂)—, —C(═NR^{gg})—, —NR^{gg}—, —O—C(R^{g})₂—, —O—C(═C(R^{ggg})₂)—, —O—C(═NR^{gg})—, —S—C(R^{g})₂—, —S—C(═C(R^{ggg})₂)—, —S—C(═NR^{gg})—, —SO—C(R^{g})₂—, —SO—C(═C(R^{ggg})₂)—, —SO—C(═NR^{gg})—, —SO—NR^{gg}—, —SO₂—C(R^{g})₂—, —SO₂—C(═C(R^{ggg})₂)—, —SO₂—C(═NR^{gg})—, —SO₂—NR^{gg}—, —SO(═NR^{gg})—C(R^{g})₂—, —SO(═NR^{gg})—C(═C(R^{ggg})₂)—, —SO(═NR^{gg})—NR^{gg}—, —CO—C(R^{g})₂—, —CO—C(═C(R^{ggg})₂)—, —CO—C(═NR^{gg})—, —CO—NR^{gg}—, —C(═S)—C(R^{g})₂—, —C(═S)—C(═C(R^{ggg})₂)—, —C(R^{g})₂—O—, —C(R^{g})₂—S—, —C(R^{g})₂—SO—, —C(R^{g})₂—SO₂—, —C(R^{g})₂—SO(═NR^{gg})—, —C(R^{g})₂—CO—, —C(R^{g})₂—C(═S)—, —C(R^{g})₂—C(R^{g})₂—, —C(R^{g})₂—C(═C(R^{ggg})₂)—, —C(R^{g})₂—C(═NR^{gg})—, —C(R^{g})₂—NR^{gg}—, —C(═C(R^{ggg})₂)—O—, —C(═C(R^{ggg})₂)—S—, —C(═C(R^{ggg})₂)—SO—, —C(═C(R^{ggg})₂)—SO₂—, —C(═C(R^{ggg})₂)—SO(═NR^{gg})—, —C(═C(R^{ggg})₂)—CO—, —C(═C(R^{ggg})₂)—C(═S)—, —C(═C(R^{ggg})₂)—C(R^{g})₂—, —C(═C(R^{ggg})₂)—C(═C(R^{ggg})₂)—, —C(═C(R^{ggg})₂)—C(═NR^{gg})—, —C(═C(R^{ggg})₂)—NR^{gg}—, —C(═NR^{gg})—O—, —C(═NR^{gg})—S—, —C(═NR^{gg})—SO—, —C(═NR^{gg})—SO₂—, —C(═NR^{gg})—CO—, —C(═NR^{gg})—C(R^{g})₂—, —C(═NR^{gg})—C(═C(R^{ggg})₂)—, —C(═NR^{gg})—C(═NR^{gg})—, —C(═NR^{gg})—NR^{gg}—, —NR^{gg}—SO—, —NR^{gg}—SO₂—, —NR^{gg}—SO(═NR^{gg})—, —NR^{gg}—CO—, —NR^{gg}—C(R^{g})₂—, —NR^{gg}—C(═C(R^{ggg})₂)—, —NR^{gg}—C(═NR^{gg})—, —CR^{ggg}═CR^{ggg}—, —CR^{ggg}═N—, —N═CR^{ggg}— or —C≡C—;
each R^{g} is independently selected from hydrogen or a halo, —OH, —NO₂, —NH₂, —N₃, —SH, —SO₂H, —SO₂NH₂, or a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton;
each R^{gg} is independently selected from hydrogen or a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton;
each R^{ggg} is independently selected from hydrogen or a halo or a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton;
or wherein optionally any two or three R^{g}, any two R^{gg}, any two or three R^{ggg}, or any two or three of R^{g}, R^{gg} and R^{ggg}, together with the atom or atoms to which they are attached, may form a saturated or unsaturated cyclic group, wherein the cyclic group may optionally be substituted; and
R² is a cyclic group substituted at the α-position, wherein R² may optionally be further substituted.

2. A compound as claimed in claim 1, wherein Q¹ and Q² are both N.

3. A compound as claimed in claim 1 or claim 2, wherein Q³ is NR^{qq}.

4. A compound as claimed in claim 3, wherein R^{qq} is independently selected from hydrogen or a C₁-C₄ alkyl or C₃-C₄ cycloalkyl group, wherein the C₁-C₄ alkyl or C₃-C₄ cycloalkyl group may optionally be substituted with one or more fluoro and/or chloro groups.

5. A compound as claimed in claim 3, wherein Q³ is NH.

6. A compound as claimed in any one of claims 1 to 5, wherein J is —S—, —SO—, —SO₂—, —SO(═NR^{jj})—, —S—C(R^{j})₂—, —SO—C(R^{j})₂—, —SO₂—C(R^{j})₂—, or —SO(═NR^{jj})—C(R^{j})₂—.

7. A compound as claimed in claim 6, wherein:
each R^{j} where present is independently selected from hydrogen or a fluoro, chloro, methyl or ethyl group, wherein any methyl or ethyl group may optionally be substituted with one or more fluoro and/or chloro groups, or any two R^{j} attached to the same carbon atom may, together with the carbon atom to which they are attached, form a 3- or 4-membered cycloalkyl group, or form an oxetanyl group, wherein the 3- or 4-membered cycloalkyl group or the oxetanyl group may optionally be substituted with one or more fluoro and/or chloro groups; and
each R^{jj} where present is selected from hydrogen, —CN, or a C₁-C₄ alkyl or C₃-C₄ cycloalkyl group, wherein the C₁-C₄ alkyl or C₃-C₄ cycloalkyl group may optionally be substituted with one or more fluoro and/or chloro groups.

8. A compound as claimed in claim 6, wherein J is —S—, —SO—, —SO₂—, —SO(═NH)—, —S—CH₂—, —SO—CH₂—, —SO₂—CH₂—, or —SO(═NH)—CH₂—.

9. A compound as claimed in claim 8, wherein J is —SO— or—SO₂—.

10. A compound as claimed in claim 9, wherein J is —SO₂—.

11. A compound as claimed in any one of claims 6 to 10, wherein a carbon or nitrogen atom of R¹ is directly attached to the sulfur atom of J.

12. A compound as claimed in claim 11, wherein R¹ is a saturated or unsaturated C₁-C₂₀ hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton.

13. A compound as claimed in claim 11 or claim 12, wherein:
R¹ is a C₁-C₁₅ alkyl, C₂-C₁₅ alkenyl or C₂-C₁₅ alkynyl group, all of which may optionally be substituted, and all of which may optionally include one, two or three heteroatoms N, O or S in their carbon skeleton; or
R¹ is a 3- to 12-membered cyclic group, wherein the cyclic group may optionally be substituted; or
R₁ is R¹⁰-L-, wherein R¹⁰ is a 3- to 12-membered cyclic group, wherein the cyclic group may optionally be substituted, wherein L is —NH— or an alkylene group, wherein the alkylene group may optionally include one or two heteroatoms independently selected from oxygen and nitrogen in its carbon skeleton, wherein the alkylene group may optionally be substituted, and wherein L contains from 1 to 10 atoms other than hydrogen or halogen.

14. A compound as claimed in any one of claims 11 to 13, wherein:
R¹ is a C₁-C₁₀ alkyl group, wherein the C₁-C₁₀ alkyl group may optionally be substituted with one or more monovalent substituents and/or divalent π-bonded substituents, and wherein the C₁-C₁₀ alkyl group may optionally include one, two or three heteroatoms independently selected from oxygen and nitrogen in its carbon skeleton; or
R¹ is a phenyl or a 5- or 6-membered heteroaryl group, wherein the phenyl or the 5- or 6-membered heteroaryl group may optionally be substituted; or
R¹ is a 8- to 10-membered fused bicyclic group, wherein a first ring in the fused bicyclic structure is a non-aromatic ring and a second ring in the fused bicyclic structure is an aromatic ring, and wherein the fused bicyclic group may optionally be substituted; or
R¹ is a 3- to 7-membered non-aromatic monocyclic group or a 7- to 12-membered non-aromatic bicyclic group, wherein the non-aromatic monocyclic group or the non-aromatic bicyclic group may optionally be substituted with one or more monovalent substituents and/or divalent π-bonded substituents; or
R¹ is R¹⁰-L-, wherein:
L is —NH— or an alkylene group, wherein the alkylene group may optionally include a single nitrogen atom in its carbon skeleton, wherein the alkylene group may optionally be substituted with one or more fluoro groups, and wherein L contains from 1 to 6 atoms other than hydrogen or halogen; and
R¹⁰ is a phenyl or a 5- or 6-membered heteroaryl group, wherein the phenyl or the 5- or 6-membered heteroaryl group may optionally be substituted; or
R¹⁰ is a 3- to 7-membered non-aromatic monocyclic group, wherein the non-aromatic monocyclic group may optionally be substituted with one or more monovalent substituents and/or divalent π-bonded substituents.

15. A compound as claimed in any one of claims 11 to 14, wherein R¹ is substituted with one or more substituents independently selected from halo; —CN; —NO₂; —N₃; —R^{β}; —OH; —OR^{β}; —SH; —SR^{β}; —SOR^{β}; —SO₂H; —SO₂R^{β}; —SO₂NH₂; —SO₂NHR^{β}; —SO₂N(R^{β})₂; —R^{α}—SH; —R^{α}—SR^{β}; —R^{α}—SOR^{β}; —R^{α}—SO₂H; —R^{α}—SO₂R^{β}; —R^{α}—SO₂NH₂; —R^{α}—SO₂NHR^{β}; —R^{α}—SO₂N(R^{β})₂; —NH₂; —NHR^{β}; —N(R^{β})₂; —N⁺(R^{β})₃; —R^{α}—NH₂; —R^{α}—NHR^{β}; —R^{α}—N(R^{β})₂; —R^{α}—N⁺(R^{β})₃; —CHO; —COR^{β}; —COOH; —COOR^{β}; —OCOR^{β}; —R^{α}—CHO; —R^{α}—COR^{β}; —R^{α}—COOH; —R^{α}—COOR^{β}; —R^{α}—OCOR^{β}; —CONH₂; —CONHR^{β}; —CON(R^{β})₂; oxo (═O); or a C₁-C₄ alkylene bridge;
wherein each —R^{α}— is independently selected from an alkylene, alkenylene or alkynylene group, wherein the alkylene, alkenylene or alkynylene group contains from 1 to 6 atoms in its backbone, wherein one or two carbon atoms in the backbone of the alkylene, alkenylene or alkynylene group may optionally be replaced by one or two heteroatoms N, O or S, wherein a single —CH₂— group in the backbone of the alkylene, alkenylene or alkynylene group may optionally be replaced by a —N⁺(R^{β})₂-group, and wherein the alkylene, alkenylene or alkynylene group may optionally be substituted with one or more halo and/or —R^{β} groups; and
wherein each —R^{β} is independently selected from a C₁-C₆ alkyl, C₂-C₆ alkenyl, C₂-C₆ alkynyl or C₂-C₆ cyclic group, or wherein any two or three —R^{β} attached to the same nitrogen atom may, together with the nitrogen atom to which they are attached, form a C₂-C₇ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) group.

16. A compound as claimed in any one of claims 11 to 15, wherein R¹ is substituted with one or more halo groups, and/or with one, two or three substituents independently selected from C₁-C₅ alkyl, C₁-C₅ haloalkyl, C₃-C₆ cycloalkyl, C₃-C₆ halocycloalkyl, C₂-C₅ alkenyl, C₂-C₅ haloalkenyl, C₅-C₆ cycloalkenyl, C₅-C₆ halocycloalkenyl, C₂-C₅ alkynyl, C₂-C₅ haloalkynyl, phenyl, halophenyl, 5- or 6-membered heteroaryl (optionally halo substituted), —R¹—CN, —R¹—N₃, —R¹—NO₂, —R¹¹—N(R¹²)₂, —R¹¹—OR¹², —R¹¹—COR¹², —R¹¹—COOR¹², —R¹¹—CON(R¹²)₂, —R¹¹—SO₂R¹², —R¹¹—SO₂N(R¹²)₂, oxo (═O), 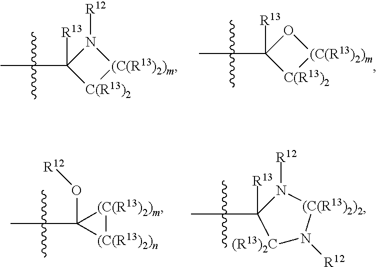 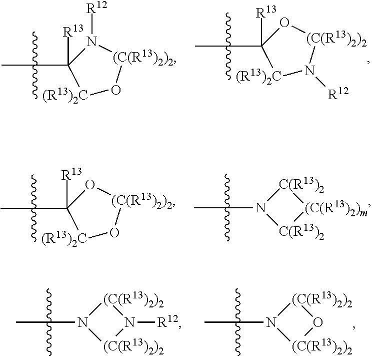 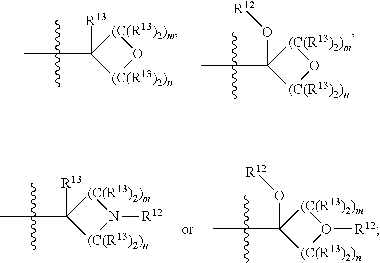
wherein R¹¹ is independently selected from a bond, C₁-C₄ alkylene, C₁-C₄ haloalkylene, C₃-C₄ cycloalkylene or C₃-C₄ halocycloalkylene;
each R¹² is independently selected from hydrogen, C₁-C₅ alkyl, C₁-C₅ haloalkyl, C₃-C₅ cycloalkyl or C₃-C₅ halocycloalkyl, or any two R¹² attached to the same nitrogen atom may together form a C₂-C₅ alkylene or C₂-C₅ haloalkylene group;
each R¹³ is independently selected from hydrogen or halo;
m is 1, 2 or 3; and
n is 1, 2 or 3.

17. A compound as claimed in any one of claims 11 to 16, wherein R¹ contains from 1 to 30 atoms other than hydrogen or halogen.

18. A compound as claimed in any one of claims 1 to 17, wherein G is —O—, —C(R^{g})₂—, or —NR^{gg}—.

19. A compound as claimed in claim 18, wherein:
each R^{g} where present is independently selected from hydrogen or a fluoro, chloro, methyl or ethyl group, wherein any methyl or ethyl group may optionally be substituted with one or more fluoro and/or chloro groups, or any two R^{g} attached to the same carbon atom may, together with the carbon atom to which they are attached, form a 3- or 4-membered cycloalkyl group, or form an oxetanyl group, wherein the 3- or 4-membered cycloalkyl group or the oxetanyl group may optionally be substituted with one or more fluoro and/or chloro groups; and
each R⁹⁹ where present is selected from hydrogen or a C₁-C₄ alkyl or C₃-C₄ cycloalkyl group, wherein the C₁-C₄ alkyl or C₃-C₄ cycloalkyl group may optionally be substituted with one or more fluoro and/or chloro groups.

20. A compound as claimed in claim 18, wherein G is —O—, —CH₂—, or —NH—.

21. A compound as claimed in any one of claims 1 to 20, wherein R² is a phenyl or a 5- or 6-membered heteroaryl group, wherein the phenyl or the heteroaryl group is substituted at the α-position, and wherein R² may optionally be further substituted.

22. A compound as claimed in claim 21, wherein the phenyl or the heteroaryl group is substituted at the α and α′ positions, and wherein R² may optionally be further substituted.

23. A compound as claimed in claim 21 or claim 22, wherein at least one substituent at the α and/or α′ positions comprises a carbon atom.

24. A compound as claimed in claim 22, wherein both substituents at the α and α′ positions comprise a carbon atom.

25. A compound as claimed in any one of claims 21 to 24, wherein —R² has a formula selected from: 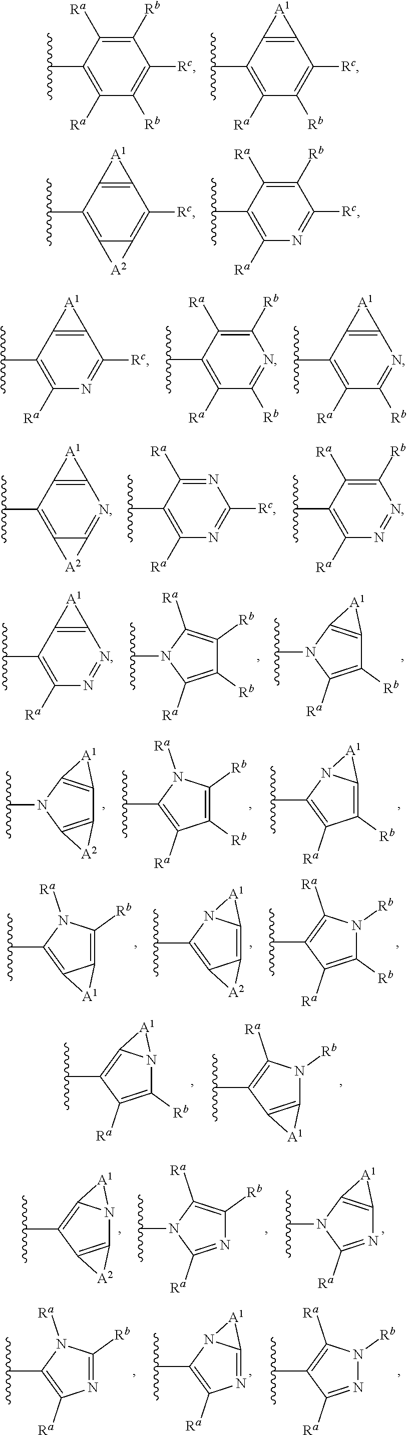 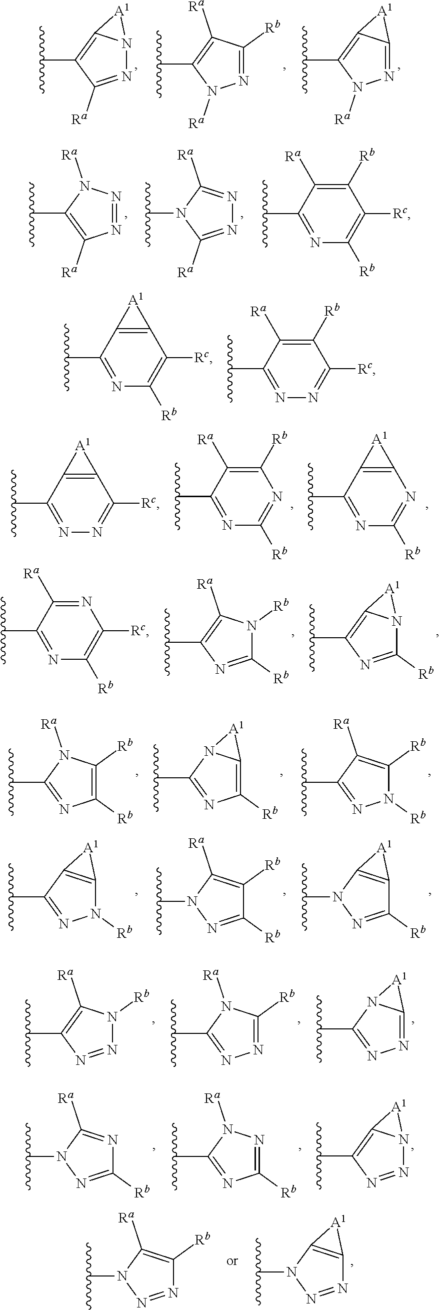
wherein:
A¹ and A² are each independently selected from a straight-chained alkylene group or a straight-chained alkenylene group, wherein one or two carbon atoms in the backbone of the alkylene or alkenylene group may optionally be replaced by one or two heteroatoms independently selected from nitrogen and oxygen, wherein any ring containing A¹ or A² is a 5- or 6-membered ring, and wherein the alkylene or alkenylene group may optionally be substituted with one or more substituents independently selected from halo, —OH, —CN, —NO₂, C₁-C₄ alkyl, C₁-C₄ haloalkyl, —O(C₁-C₄ alkyl) or —O(C₁-C₄ haloalkyl);
each R^{a} is independently selected from hydrogen, halo, —R^{aa}, —OR^{aa} or —COR^{aa}, provided that at least one R^{a} is —R^{aa}, —OR^{aa} or —COR^{aa};
each R^{b} is independently selected from hydrogen, halo, —NO₂, —CN, —R^{aa}, —OR^{aa} or —COR^{aa};
provided that any R^{a} or R^{b} that is directly attached to a ring nitrogen atom is not halo, —NO₂, —CN, or —OR^{aa};
each R^{aa} is independently selected from a C₁-C₆ alkyl, C₂-C₆ alkenyl, C₂-C₆ alkynyl or a 3- to 7-membered cyclic group, wherein each C₁-C₆ alkyl, C₂-C₆ alkenyl, or C₂-C₆ alkynyl group is optionally substituted with one or more substituents independently selected from halo, —OH, —CN, —NO₂, —O(C₁-C₄ alkyl) or —O(C₁-C₄ haloalkyl), and wherein each 3- to 7-membered cyclic group is optionally substituted with one or more substituents independently selected from halo, —OH, —NH₂, —CN, —NO₂, —B¹, —CH₂B¹, —OB¹, —OCH₂B¹, —NHB¹, —N(B¹)₂, —CONH₂, —CONHB¹, —CON(B¹)₂, —NHCOB¹, —NB¹COB¹, or —B¹¹—;
each B¹ is independently selected from a C₁-C₆ alkyl, C₂-C₆ alkenyl, C₂-C₆ alkynyl, C₃-C₁₀ cycloalkyl, C₅-C₁₀ cycloalkenyl, C₆-C₁₀ aryl, or a 4- to 10-membered heterocyclic group containing one or two ring heteroatoms N and/or O, or two B¹ together with the nitrogen atom to which they are attached may form a 4- to 10-membered heterocyclic group containing one or two ring heteroatoms N and/or O, wherein any B¹ may optionally be halo-substituted and/or substituted with one or two substituents independently selected from —OH, —NH₂, —B¹², —OB¹², —NHB¹² or —N(B¹²)₂;
each B¹¹ is independently selected from a C₁-C₈ alkylene or C₂-C₈ alkenylene group, wherein one or two carbon atoms in the backbone of the alkylene or alkenylene group may optionally be replaced by one or two heteroatoms N and/or O, and wherein the alkylene or alkenylene group may optionally be halo-substituted and/or substituted with one or two substituents independently selected from —OH, —NH₂, —B¹², —OB¹², —NHB¹² or —N(B¹²)₂;
each B¹² is independently selected from a C₁-C₃ alkyl or C₁-C₃ haloalkyl group;
each R^{c} is selected from hydrogen, halo, —OH, —NO₂, —CN, —R^{cc},—R^{c}x, —OR^{cc}, —COR^{cc}, —COOR^{cc}, —CONH₂, —CONHR^{cc}, —CON(R^{cc})₂, —C(═NH)R^{cc}, —C(═NH)NH₂, —C(═NH)NHR^{cc}, —C(═NH)N(R^{cc})₂, —C(═NR^{cc})R^{cc}, —C(═NR^{cc})NHR^{cc}, —C(═NR^{cc})N(R^{cc})₂, —C(═NOH)R^{cc} or —C(═NOR^{cc})R^{cc};
each R^{cc} is independently selected from C₁-C₄ alkyl, C₁-C₄ haloalkyl, C₃-C₄ cycloalkyl or C₃-C₄ halocycloalkyl, or any two R^{cc} attached to the same nitrogen atom may, together with the nitrogen atom to which they are attached, form a 3- to 6-membered saturated heterocyclic group, wherein the 3- to 6-membered saturated heterocyclic group is optionally halo substituted; and
each R^{cx} is selected from a 3- to 7-membered cyclic group, wherein the 3- to 7-membered cyclic group is optionally halo substituted.

26. A compound as claimed in any one of claims 21 to 25, wherein R² contains from 10 to 35 atoms other than hydrogen or halogen.

27. A compound selected from the group consisting of:

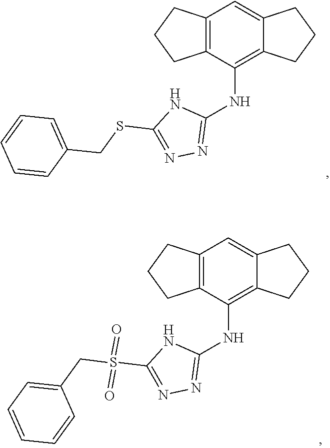 [SMILES: c1ccc(CSc2nnc(Nc3c4c(cc5c3CCC5)CCC4)[nH]2)cc1 ; O=S(=O)(Cc1ccccc1)c1nnc(Nc2c3c(cc4c2CCC4)CCC3)[nH]1]

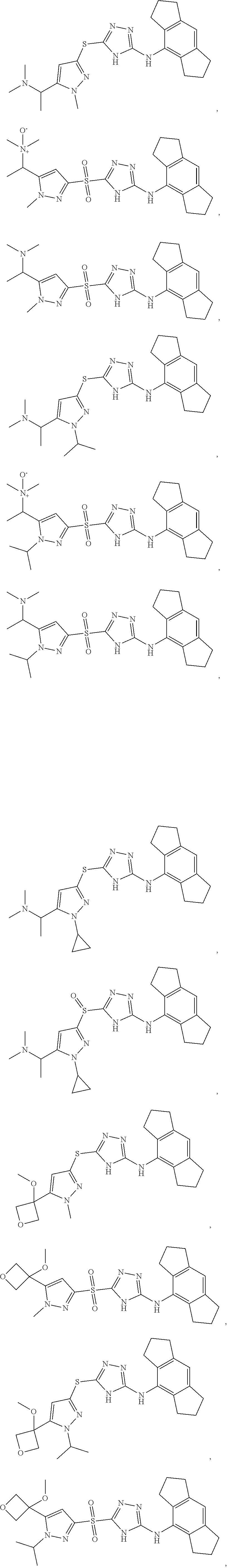

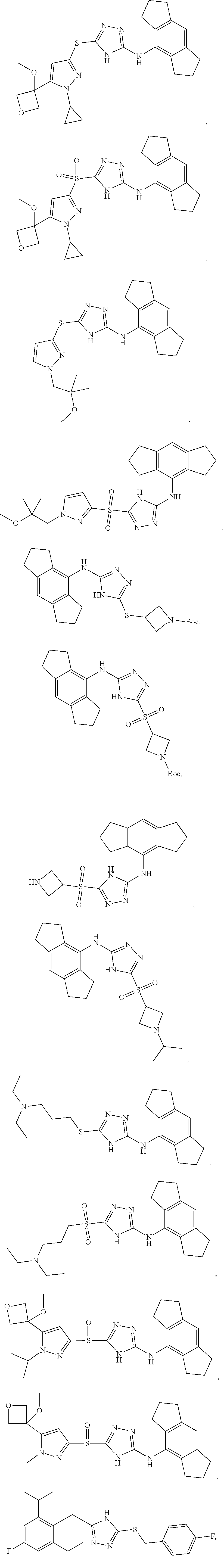

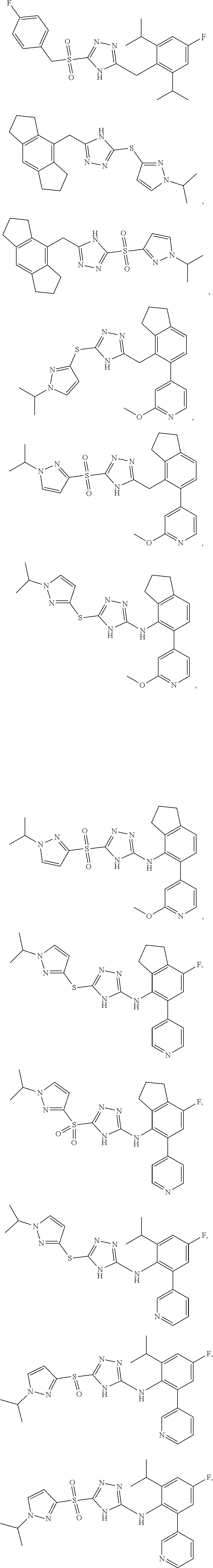

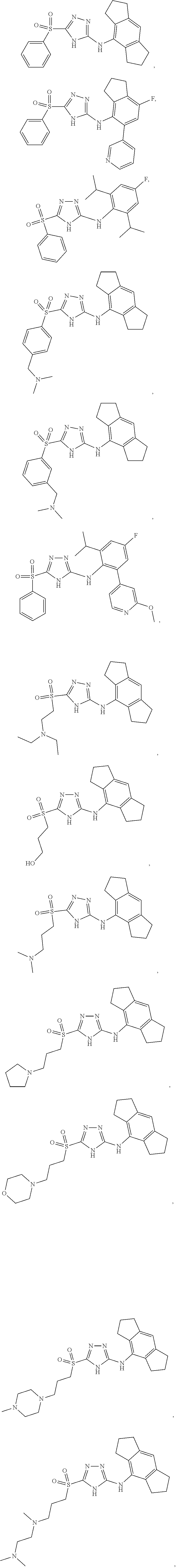

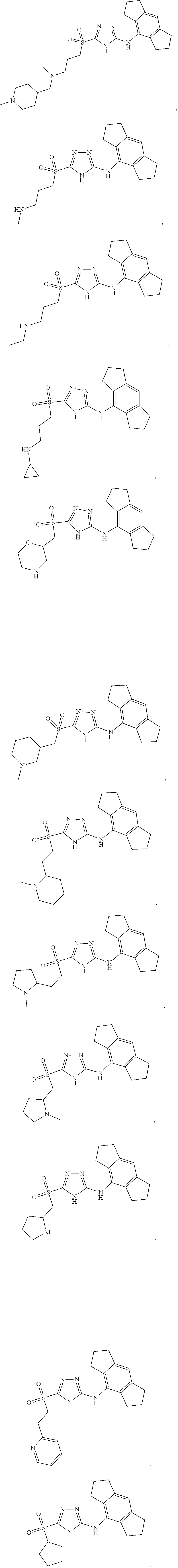

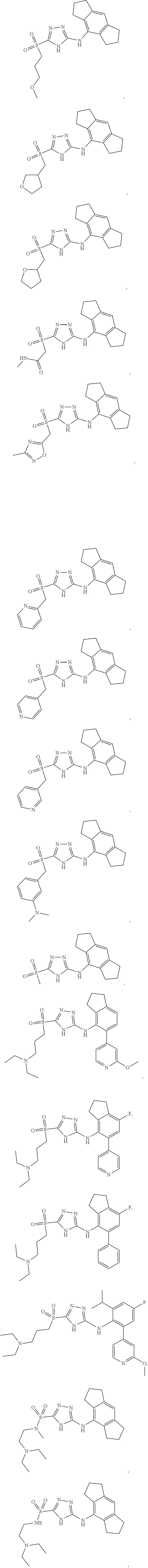

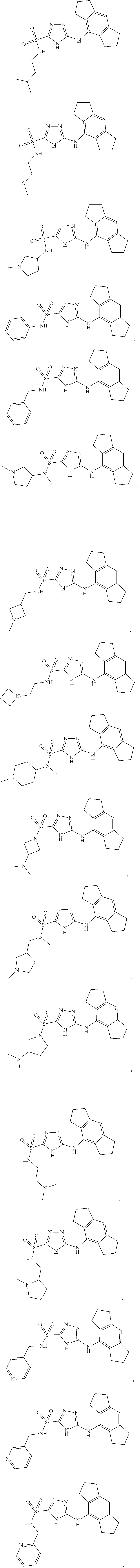

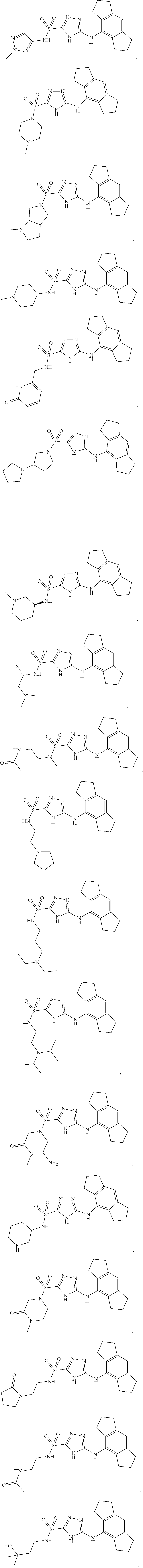

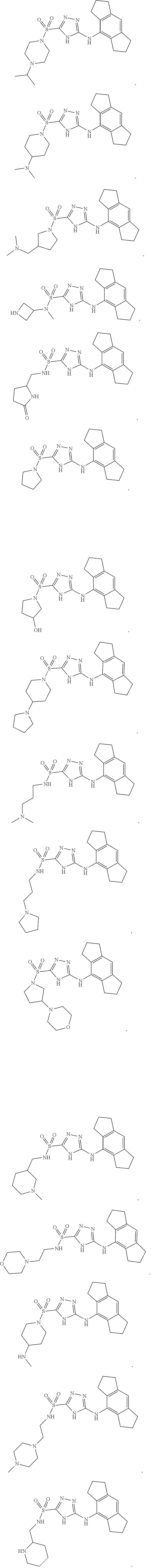

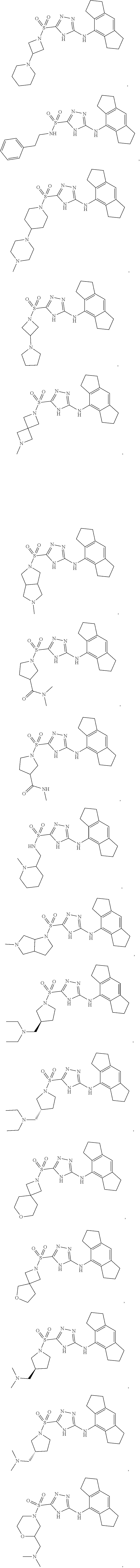

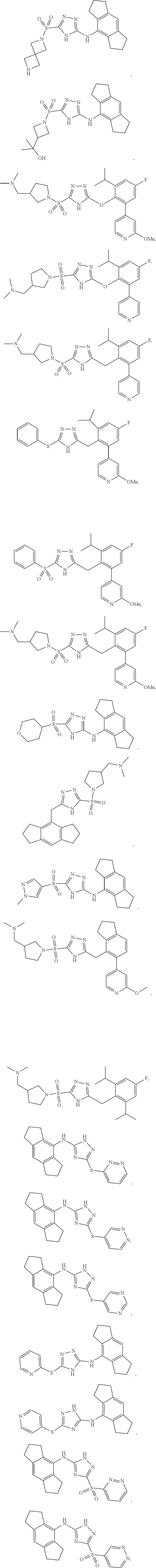

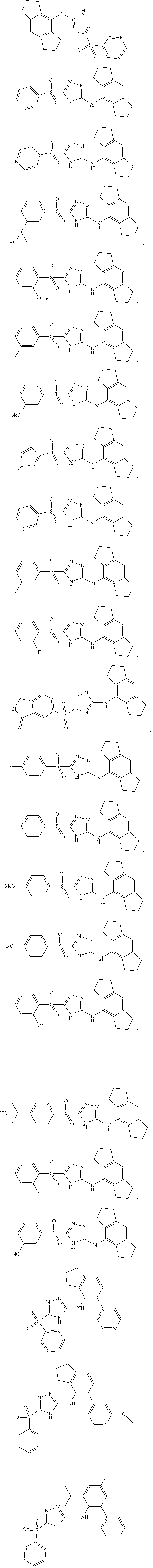

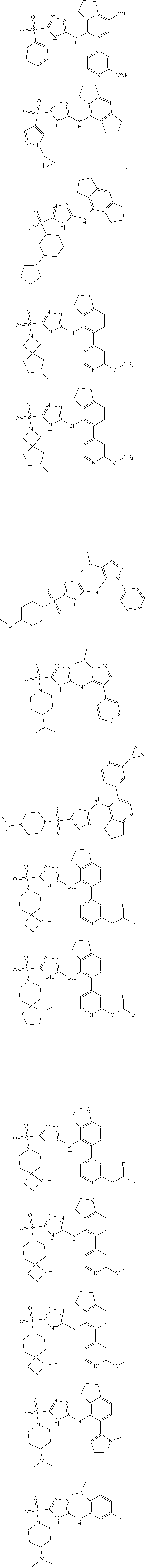

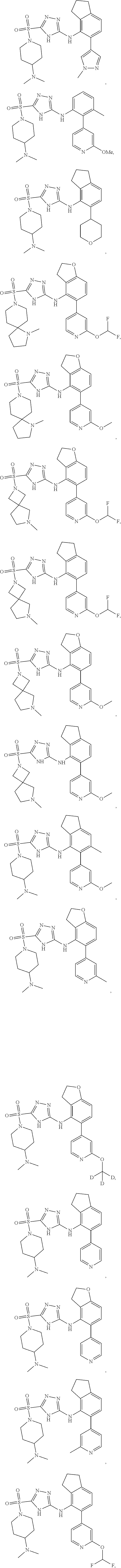

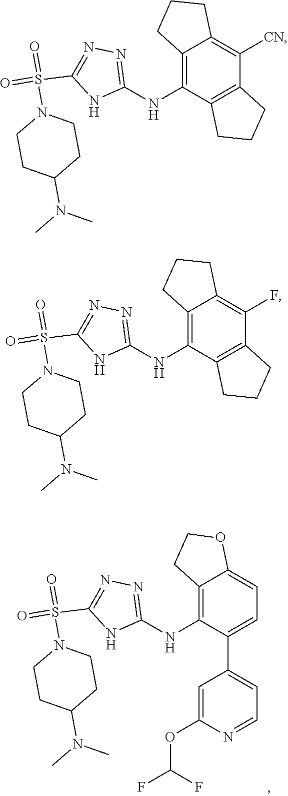 [SMILES: CN(C)C1CCN(S(=O)(=O)c2nnc(Nc3c4c(c(C#N)c5c3CCC5)CCC4)[nH]2)CC1 ; CN(C)C1CCN(S(=O)(=O)c2nnc(Nc3c4c(c(F)c5c3CCC5)CCC4)[nH]2)CC1 ; CN(C)C1CCN(S(=O)(=O)c2nnc(Nc3c(-c4ccnc(OC(F)F)c4)ccc4c3CCO4)[nH]2)CC1]

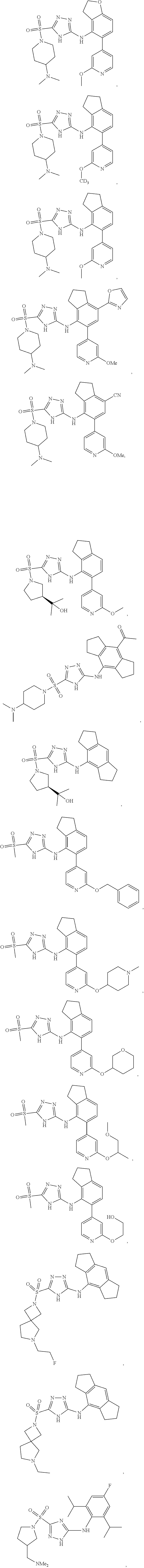

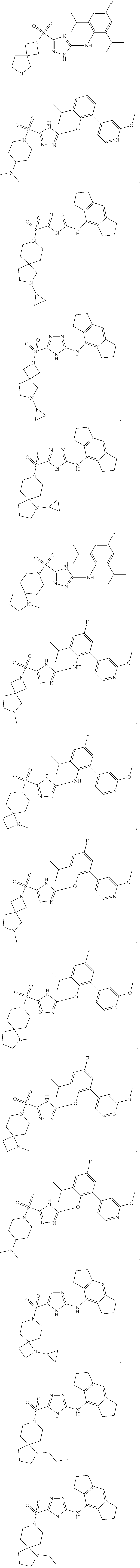

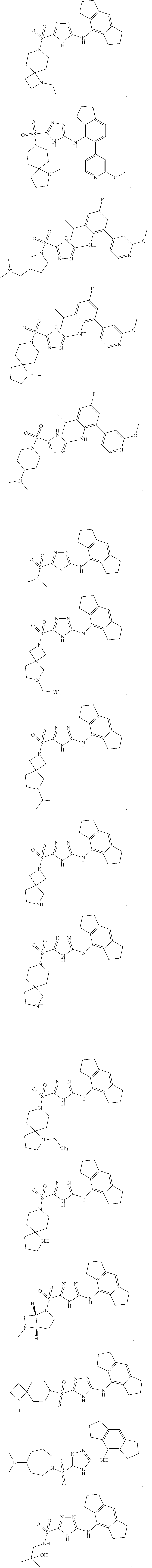

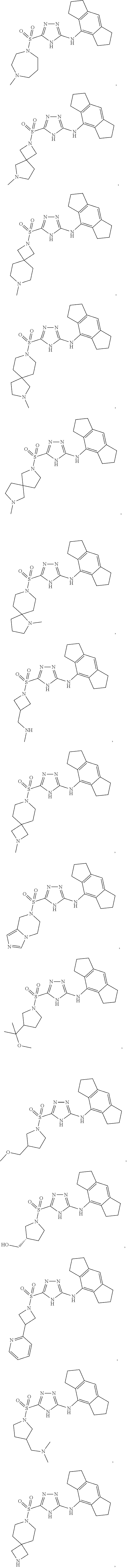

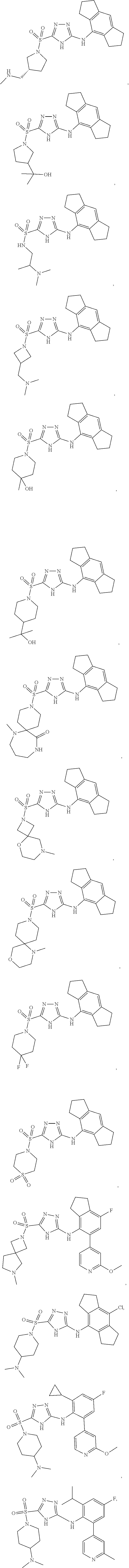

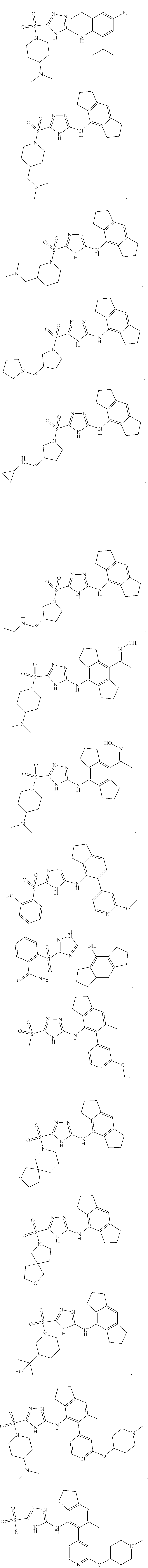

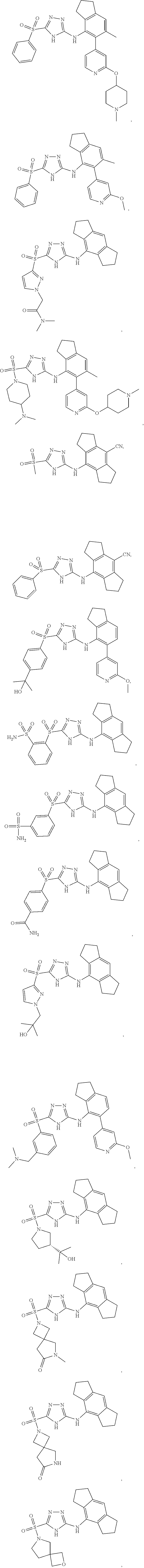

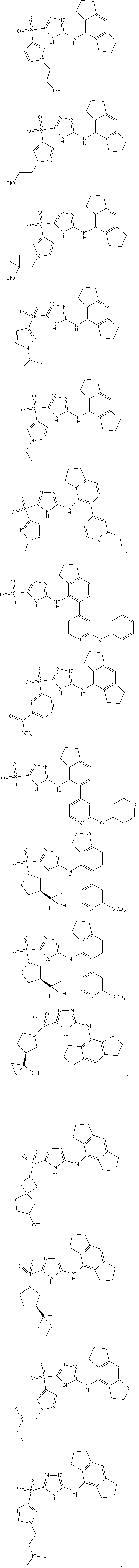

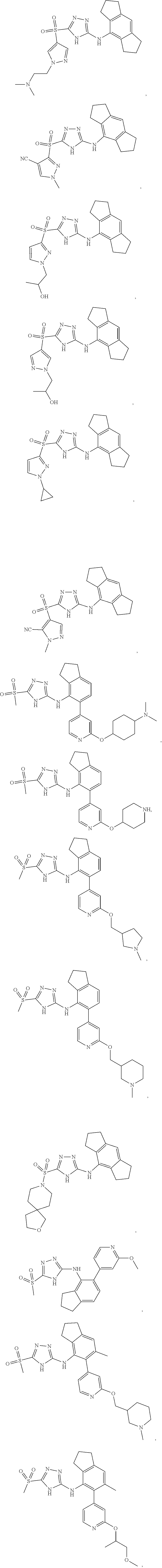

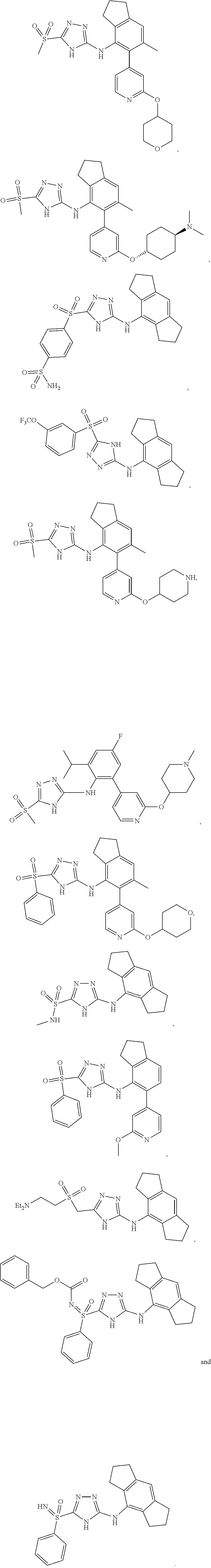

28. A pharmaceutically acceptable salt, solvate or prodrug of a compound as claimed in any one of claims 1 to 27.

29. A pharmaceutical composition comprising a compound as claimed in any one of claims 1 to 27, or a pharmaceutically acceptable salt, solvate or prodrug as claimed in claim 28, and a pharmaceutically acceptable excipient.

30. A compound as claimed in any one of claims 1 to 27, or a pharmaceutically acceptable salt, solvate or prodrug as claimed in claim 28, or a pharmaceutical composition as claimed in claim 29, for use in medicine.

31. A compound, pharmaceutically acceptable salt, solvate, prodrug or pharmaceutical composition as claimed in claim 30, for use in the treatment or prevention of a disease, disorder or condition, wherein the disease, disorder or condition is responsive to NLRP3 inhibition.

32. A compound, pharmaceutically acceptable salt, solvate, prodrug or pharmaceutical composition as claimed in claim 30 or claim 31, for use in the treatment or prevention of a disease, disorder or condition, wherein the disease, disorder or condition is selected from:
(i) inflammation;
(ii) an auto-immune disease;
(iii) cancer;
(iv) an infection;
(v) a central nervous system disease;
(vi) a metabolic disease;
(vii) a cardiovascular disease;
(viii) a respiratory disease;
(ix) a liver disease;
(x) a renal disease;
(xi) an ocular disease;
(xii) a skin disease;
(xiii) a lymphatic condition;
(xiv) a psychological disorder;
(xv) graft versus host disease;
(xvi) allodynia; and
(xvii) any disease where an individual has been determined to carry a germline or somatic non-silent mutation in NLRP3.

33. A compound, pharmaceutically acceptable salt, solvate, prodrug or pharmaceutical composition as claimed in claim 30 or claim 31, for use in the treatment or prevention of a disease, disorder or condition, wherein the disease, disorder or condition is selected from:
(i) cryopyrin-associated periodic syndromes (CAPS);
(ii) Muckle-Wells syndrome (MWS);
(iii) familial cold autoinflammatory syndrome (FCAS);
(iv) neonatal onset multisystem inflammatory disease (NOMID);
(v) familial Mediterranean fever (FMF);
(vi) pyogenic arthritis, pyoderma gangrenosum and acne syndrome (PAPA);
(vii) hyperimmunoglobulinemia D and periodic fever syndrome (HIDS);
(viii) Tumour Necrosis Factor (TNF) Receptor-Associated Periodic Syndrome (TRAPS);
(ix) systemic juvenile idiopathic arthritis;
(x) adult-onset Still's disease (AOSD);
(xi) relapsing polychondritis;
(xii) Schnitzler's syndrome;
(xiii) Sweet's syndrome;
(xiv) Behcet's disease;
(xv) anti-synthetase syndrome;
(xvi) deficiency of interleukin 1 receptor antagonist (DIRA); and
(xvii) haploinsufficiency of A20 (HA20).

34. A method of inhibiting NLRP3, the method comprising the use of a compound as claimed in any one of claims 1 to 27, or a pharmaceutically acceptable salt, solvate or prodrug as claimed in claim 28, or a pharmaceutical composition as claimed in claim 29, to inhibit NLRP3.
